(19) United States
(12) Patent Application Publication
(10) Pub. No.: US 2016/0145247 A1
(43) Pub. Date: May 26, 2016

(54) Aminomethyl-Biaryl Derivatives Complement Factor D inhibitors and uses thereof

(71) Applicants: David B. BELANGER, Cambridge, MA (US); Stefanie FLOHR, Basel (CH); Christine Fang GELIN, San Diego, CA (US); Keith JENDZA, Cambridge, MA (US); Nan JI, Cambridge, MA (US); Donglei LIU, Cambridge, MA (US); Edwige Lilliane Jeanne LORTHIOIS, Basel (CH); Rakeshri Ganesh KARKI, Cambridge, MA (US); Nello MAINOLFI, Cambridge, MA (US); James J. POWERS, Cambridge, MA (US); Stefan Andreas RANDL, Frankfurt am Main (DE); Olivier ROGEL, Hesingue (FR); Anna VULPETTI, Basel (CH); Taeyoung YOON, Cambridge, MA (US)

(72) Inventors: David Belanger, Basel (CH); Stefanie Flohr, Lorrach (DE); Christine Fang Gelin, San Diego, CA (US); Keith Jendza, Boston, MA (US); Nan Ji, Arlington, MA (US); Rajeshri Ganesh Karki, Somerville, MA (US); Donglei Liu, Dover, MA (US); Edwoge Liliane Jeanne LORTHIOIS, Niffer (FR); Nello Mainolfi, Boston, MA (US); James J. Powers, Waltham, MA (US); Stefan Andreas Randi, Frnakfuurt am Main (DE); Oliver Rogel, Hesingue (FR); Anna Vulpetti, Basel (CH); Taeyoung YOON, Seoul (KR)

(73) Assignee: NOVARTIS AG, Basel (CH)

(21) Appl. No.: 14/904,693

(22) PCT Filed: Jul. 17, 2014
(86) PCT No.: PCT/US14/47106

§ 371 Date: Jan. 12, 2016

Related U.S. Application Data
(60) Provisional application No. 61/847,842, filed on Jul. 18, 2013.
(60) Provisional application No. 62/012,002, filed on Jun. 13, 2014.

(51) Int. Cl. C07D 417/12; C07C 237/42; C07D 263/32; C07D 213/56; C07D 231/12; C07D 307/16; C07D 213/81; C07D 277/56; C07D 213/82; C07D 233/90; C07D 405/12; C07C 311/37; C07D 239/26; C07D 309/14; C07D 309/06; C07C 229/34; C07D 311/42; C07D 317/60; C07D 231/56; C07D 257/04; C07D 265/30; C07D 267/10; C07D 215/12; C07D 205/04; C07D 305/06; C07C 237/40

(52) U.S. Cl. CPC C07D 417/12; C07C 237/40; C07C 237/42; C07D 263/32; C07D 213/56; C07D 231/12; C07D 307/16; C07D 213/81; C07D 277/56; C07D 213/82; C07D 233/90; C07D 405/12; C07C 311/37; C07D 239/26; C07D 309/14; C07D 309/06; C07C 229/34; C07D 311/42; C07D 317/60; C07D 231/56; C07D 257/04; C07D 265/30; C07D 267/10; C07D 215/12; C07D 205/04; C07D 305/06

(57) ABSTRACT

The present invention provides a compound of formula (I), a method for manufacturing the compounds of the invention, and its therapeutic uses. The present invention further provides a combination of pharmacologically active agents and a pharmaceutical composition.

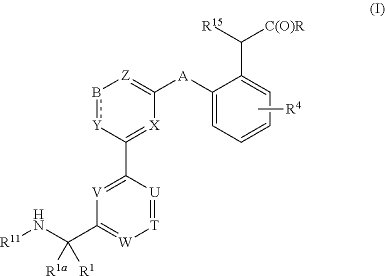

### FIELD OF THE INVENTION

The invention relates to the inhibition of the complement alternative pathway and particularly to inhibition of Factor D, in patients suffering from conditions and diseases associated with complement alternative pathway activation such as age-related macular degeneration, diabetic retinopathy and related ophthalmic diseases.

### BACKGROUND OF THE INVENTION

The complement system is a crucial component of the innate immunity system and comprises a group of proteins that are normally present in an inactive state. These proteins are organized in three activation pathways: the classical, the lectin, and the alternative pathways (V. M. Holers, In Clinical Immunology: Principles and Practice, ed. R. R. Rich, Mosby Press; 1996, 363-391). Molecules from microorganisms, antibodies or cellular components can activate these pathways resulting in the formation of protease complexes known as the C3-convertase and the C5-convertase. The classical pathway is a calcium/magnesium-dependent cascade, which is normally activated by the formation of antigen-antibody complexes. It can also be activated in an antibody-independent manner by the binding of C-reactive protein complexed to ligand and by many pathogens including gram-negative bacteria. The alternative pathway is a magnesium-dependent cascade which is activated by deposition and activation of C3 on certain susceptible surfaces (e.g., cell wall polysaccharides of yeast and bacteria, and certain biopolymer materials).

Factor D may be a suitable target for the inhibition of this amplification of the complement pathways because its plasma concentration in humans is very low (about 1.8 μg/mL), and it has been shown to be the limiting enzyme for activation of the alternative complement pathway (P. H. Lesavre and H. J. Müller-Eberhard. J. Exp. Med., 1978; 148: 1498-1510; J. E. Volanakis et al., New Eng. J. Med., 1985; 312:395-401).

Macular degeneration is a clinical term that is used to describe a family of diseases that are characterized by a progressive loss of central vision associated with abnormalities of Bruch's membrane, the choroid, the neural retina and/or the retinal pigment epithelium. In the center of the retina is the macula lutea, which is about ⅓ to ½ cm in diameter. The macula provides detailed vision, particularly in the center (the fovea), because the cones are higher in density and because of the high ratio of ganglion cells to photoreceptor cells. Blood vessels, ganglion cells, inner nuclear layer and cells, and the plexiform layers are all displaced to the side (rather than resting above the photoreceptor cells), thereby allowing light a more direct path to the cones. Under the retina is the choroid, a part of the uveal tract, and the retinal pigmented epithelium (RPE), which is between the neural retina and the choroid. The choroidal blood vessels provide nutrition to the retina and its visual cells.

Age-related macular degeneration (AMD), the most prevalent form of macular degeneration, is associated with progressive loss of visual acuity in the central portion of the visual field, changes in color vision, and abnormal dark adaptation and sensitivity. Two principal clinical manifestations of AMD have been described as the dry, or atrophic, form and the neovascular, or exudative, form. The dry form is associated with atrophic cell death of the central retina or macula, which is required for fine vision used for activities such as reading, driving or recognizing faces. About 10-20% of these AMD patients progress to the second form of AMD, known as neovascular AMD (also referred to as wet AMD).

Neovascular AMD is characterized by the abnormal growth of blood vessels under the macula and vascular leakage, resulting in displacement of the retina, hemorrhage and scarring. This results in a deterioration of sight over a period of weeks to years. Neovascular AMD cases originate from intermediate or advanced dry AMD. The neovascular form accounts for 85% of legal blindness due to AMD. In neovascular AMD, as the abnormal blood vessels leak fluid and blood, scar tissue is formed that destroys the central retina.

The new blood vessels in neovascular AMD are usually derived from the choroid and are referred to as choroidal neovascularizaton (CNV). The pathogenesis of new choroidal vessels is poorly understood, but such factors as inflammation, ischemia, and local production of angiogenic factors are thought to be important. A published study suggests that CNV is caused by complement activation in a mouse laser model (Bora P. S., J. Immunol. 2005; 174; 491-497).

Human genetic evidence implicates the involvement of the complement system, particularly the alternative pathway, in the pathogenesis of Age-related Macular Degeneration (AMD). Significant associations have been found between AMD and polymorphisms in complement factor H (CFH) (Edwards A O, et al. Complement factor H polymorphism and age-related macular degeneration. Science. 2005 Apr. 15; 308(5720):421-4; Hageman G S, et al Acommon haplotype in the complement regulatory gene factor H (HF1/CFH) predisposes individuals to age-related macular degeneration. Proc Natl Acad Sci USA. 2005 May 17; 102(20):7227-32; Haines J L, et al. Complement factor H variant increases the risk of age-related macular degeneration. Science. 2005 Apr. 15; 308(5720):419-21; Klein R J, et al Complement factor H polymorphism in age-related macular degeneration. Science. 2005 Apr. 15; 308(5720):385-9; Lau L I, et al. Association of the Y402H polymorphism in complement factor H gene and neovascular age-related macular degeneration in Chinese patients. Invest Ophthalmol Vis Sci. 2006 August; 47(8):3242-6; Simonelli F, et al.; .Br J Ophthalmol. 2006 September; 90(9):1142-5; and Zareparsi S, et al Strong association of the Y402H variant in complement factor H at 1q32 with susceptibility to age-related macular degeneration. Am J Hum Genet. 2005 July; 77(1):149-53), complement factor B (CFB) and complement C2 (Gold B, et al. Variation in factor B (BF) and complement component 2 (C2) genes is associated with age-related macular degeneration. Nat Genet. 2006 April; 38(4):458-62 and Jakobsdottir J, et al. C2 and CFB genes inage-related maculopathy and joint action with CFH and LOC387715 genes. PLoS One. 2008 May 21; 3(5):e2199), and most recently in complement C3 (Despriet D D, et al Complement component C3 and risk of age-related macular degeneration. Ophthalmology. 2009 March; 116(3):474-480.e2; Mailer J B, et al Variation in complement factor 3 is associated with risk of age-related macular degeneration. Nat Genet. 2007 October; 39(10):1200-1 and Park K H, et al Complement component 3 (C3) haplotypes and risk of advanced age-related macular degeneration. Invest Ophthalmol Vis Sci. 2009 July; 50(7):3386-93. Epub 2009 Feb. 21). Taken together, the genetic variations in the alternative pathway components CFH, CFB, and C3 can predict clinical outcome in nearly 80% of cases.

Currently there is no proven medical therapy for dry AMD and many patients with neovascular AMD become legally blind despite current therapy with anti-VEGF agents such as Lucentis. Thus, it would be desirable to provide therapeutic agents for the treatment or prevention of complement mediated diseases and particularly for the treatment of AMD.

### SUMMARY OF THE INVENTION

The present invention provides compounds that modulate, and preferably inhibit, activation of the alternative complement pathway. In certain embodiments, the present invention provides compounds that modulate, and preferably inhibit, Factor D activity and/or Factor D mediated complement pathway activation. Such Factor D modulators are preferably high affinity Factor D inhibitors that inhibit the catalytic activity of complement Factor Ds, such as primate Factor D and particularly human Factor D.

The compounds of the present invention inhibit or suppress the amplification of the complement system caused by C3 activation irrespective of the inital mechanism of activation (including for example activation of the classical, lectin or alternative pathways).

Various embodiments of the invention are described herein. It will be recognized that features specified in each embodiment may be combined with other specified features to provide further embodiments.

Within certain aspects, Factor D modulators provided herein are compounds of Formula I and salts thereof:

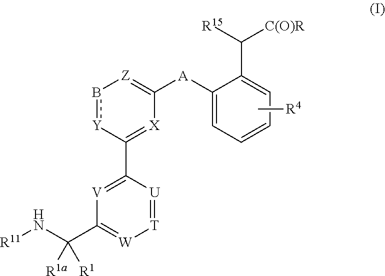

In another embodiment, the invention provides a pharmaceutical composition comprising a therapeutically effective amount of a compound according to the definition of formula (I) or subformulae thereof and one or more pharmaceutically acceptable carriers.

In another embodiment, the invention provides a combination, in particular a pharmaceutical combination, comprising a therapeutically effective amount of the compound according to the definition of formula (I) or subformulae thereof and one or more therapeutically active.

The invention further provides methods of treating or preventing complement mediated diseases, the method comprising the steps of identifying a patient in need of complement modulation therapy and administering a compound of Formula (I) or a subformulae thereof.

Complement mediated diseases include ophthalmic diseases (including early or neovascular age-related macular degeneration and geographic atrophy), autoimmune diseases (including arthritis, rheumatoid arthritis), Respiratory diseases, cardiovascular diseases.

Other aspects of the invention are discussed infra.

### DETAILED DESCRIPTION OF THE INVENTION

As noted above, the present invention provides compounds that modulate Factor D activation and/or Factor D-mediated signal transduction of the complement system. Such compounds may be used in vitro or in vivo to modulate (preferably inhibit) Factor D activity in a variety of contexts.

In a first embodiment, the invention provides compounds of Formula I and pharmaceutically acceptable salts thereof, which modulate the alternative pathway of the complement system. Compounds of Formula I are represented by the structure:

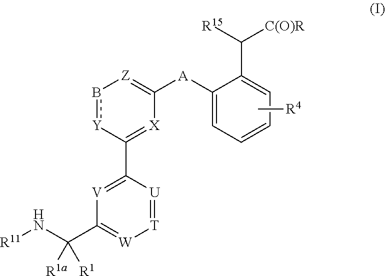

or a salt thereof, wherein

A is —C(O)NH—, —C≡C—, —CH₂CH₂—, S(O)₂N(H)—, or —CHR¹⁰O—, wherein the carbon or sulfur is attached to the ring comprising X, Y and Z; or

A is —N(R¹⁶)CH₂— or —OCH₂—, wherein the nitrogen or oxygen is attached to the ring comprising X, Y and Z; or

R is hydroxy, amino or C₁-C₄alkoxy;

R¹ is hydrogen, phenyl, C₃-C₆cycloalkyl, amido, halo C₁-C₆alkyl or C₁-C₆alkyl optionally substituted by hydroxy, C₃-C₆cycloalkyl, C₁-C₄alkoxy or cyano;

R^{1a} is hydrogen or C₁-C₄alkyl, or CR¹R^{1a} taken in combination form a carbonyl (C═O), imine (C═NH) or a 3-6 member cycloalkyl;

R^{1a} is absent and CR¹ and R¹¹, taken in combination, form a saturated, unsaturated or aromatic 4, 5 or 6 member azacycle;

T is CR² or N;

U is CR¹⁴ or N;

V is CR¹² or N;

W is CR¹³ or N, wherein 0, 1, or 2 of T, U, V and W are N; or

V is N, W is S, T is absent and U is CR¹⁴;

B is CR³ or N;

X is CR⁶ or N;

Y is CR⁵ or N;

Z is CR⁷ or N, wherein 0 or 1 of B, X, Y and Z are nitrogen; or

X is N, B is CR³ and one of Y or Z is S or N(H) and other of Y or Z is absent;

R² is hydrogen, C₁-C₄alkyl or halogen;

R³ is hydrogen, halogen, hydroxy, cyano, amino, NHR⁸, N(R⁸)₂, N(R⁸)C(O)R⁹, —C(O)NHR⁸, —C(O)N(R⁸)₂, OR⁹, S(O)₂R⁹, C₁-C₆alkyl, C₂-C₆alkenyl, C₂-C₆alkynyl, haloC₁-C₆alkyl, C₃-C₆cycloalkyl, phenyl, heterocycloalkyl having 4 to 7 ring atoms and 1, 2, or 3 ring heteroatoms independently selected from N, O and S, and heteroaryl having 5, 6, 9 or 10 ring atoms and 1, 2, or 3 ring heteroatoms independently selected from N, O and S, wherein each heterocycloalkyl, heteroaryl, phenyl is optionally substituted with 0, 1, 2, or 3 substituents independently selected from C₁-C₄alkyl, C₁-C₄alkoxy, C₁-C₄alkoxy C₁-C₄alkyl, halogen, or C₃-C₆cycloalkyl, wherein each heterocycloalkyl or heteroaryl is optionally further substituted by 0 or 1 phenyl groups and wherein each alkyl, alkenyl, alkynyl, haloalkyl and cycloalkyl group is optionally substituted with 0, 1, or 2 substituents independently selected from the group consisting of hydroxy, C₃-C₆cycloalkyl, amino, NHR⁸, N(R⁸)₂, OR⁹, 5 or 6 member heteroaryl having 1 or 2 ring heteroatoms independently selected from N, O and S, and heterocycloalkyl having 4 to 7 ring atoms and 1, 2, or 3 ring heteroatoms independently selected from N, O and S, which heteroaryl or heterocycloalkyl is substituted with 0, 1, or 2 independently selected C1-C₄alkyl substituents;
R⁴ represents 0, 1, or 2 substituents independently selected from halogen, cyano, C₁-C₄alkyl, C₁-C₄alkoxy, C₃-C₆cycloalkyl, C₃-C₆cycloalkyl C₁-C₄-alkyl, C(O)NH₂, NHC(O)C₁-C₄alkyl, CH₂NHC(O)C₁-C₄alkyl, amino, mono- and di-C₁-C₄alkylamino and hydroxy C₁-C₄alkyl;

R⁵ is hydrogen, halogen, cyano, C₁-C₄alkyl or C₁-C₄alkoxy;

R⁶ is hydrogen, halogen, C₁-C₄alkyl or C₁-C₄alkoxy;

R⁷ is hydrogen, halogen, C₁-C₄alkyl, C₁-C₄alkoxy, haloC₁-C₄alkyl or haloC₁-C₄alkoxy, or

R⁷ is phenyl or a 5 or 6 member heteroaryl having 1, 2, or 3 ring heteroatoms selected from N, O or S; each of which is optionally substituted by 0, 1, or 2 substituents selected from C₁-C₄alkyl, amino C₁-C₄alkyl, hydroxy C₁-C₄alkyl, halogen, C₁-C₄alkoxy, hydroxy, amino or mono- or di-C₁-C₄alkylamino; or

R³ and either R⁵ or R⁷ taken in combination form a —O(CH₂)ₙO— group wherein n is 1 or 2; or

R³ and R⁷ taken in combination with the atoms to which they are attached form a 5 or 6 member aromatic heterocycle having 1 or 2 ring heteroatoms selected from N, O or S and which is optionally substituted with C₁-C₄alkyl, C(O)C₁-C₄alkyl, C(O)NH2, C(O)NHC₁-C₄alkyl, C(O)N(C₁-C₄alkyl)₂, S(O)₂C₁-C₄alkyl, S(O)₂C3-C₆cycloalkyl, optionally substituted S(O)₂phenyl, where the phenyl is optionally substituted with 0, 1, or 2 C₁-C₄alkyl, or C₁-C₄alkoxy or C₁-C₄alkoxy;

R⁸ is independently selected at each occurrence from the group consisting of hydrogen, C1-C₆alkyl, haloC₁-C₆alkyl, C₃-C₆cycloalkyl, benzyl, C₁-C₄alkanoyl, benzoyl, phenyl, 4 to 6 member heterocycloalkyl, heteroaryl, wherein C₁-C₆alkyl or haloC₁-C₆alkyl is optionally substituted with C₁-C₄alkoxy, C₃-C₆cycloalkyl, cyano, 4 to 6 member heterocycloalkyl or heteroaryl, wherein phenyl or benzyl are optionally substituted with 0, 1, or 2 substituents selected from C₁-C₄alkyl, haloC₁-C₄alkyl, CH₂CO₂H, C₃-C₆cycloalkyl or C₁-C₄alkoxy, and wherein each heterocycloalkyl or heteroaryl is optionally substituted by 0, 1, or 2 substituents independently selected from C₁-C₄alkyl, CO₂ C₁-C₄alkyl, C(O)NH C₁-C₄alkyl, C₃-C₆cycloalkyl, C₁-C₄alkoxy or a fused benzo ring, and wherein each cycloalkyl is optionally substituted with 0, 1, or 2 independently selected halogen or C₁-C₄alkyl;

R⁹ is independently selected at each occurrence from the group consisting of hydrogen, C1-C₆alkyl, haloC₁-C₆alkyl, C₃-C₆cycloalkyl, benzyl, benzoyl, phenyl, 4 to 6 member heterocycloalkyl, heteroaryl, wherein C₁-C₆alkyl or haloC₁-C₆alkyl is optionally substituted with C₁-C₄alkoxy, C₃-C₆cycloalkyl, C₃-C₆cycloalkyl substituted with C₁-C₄alkyl, cyano, 4 to 6 member heterocycloalkyl or heteroaryl, wherein phenyl or benzyl are optionally substituted with 0, 1, or 2 substituents selected from C₁-C₄alkyl, CH₂CO₂H, C₃-C₆cycloalkyl or C₁-C₄alkoxy, and wherein each heterocycloalkyl or heteroaryl are optionally substituted by 0, 1, or 2 substituents independently selected from C₁-C₄alkyl, CO₂ C₁-C₄alkyl, C(O)NH C₁-C₄alkyl, C₃-C₆cycloalkyl, C₁-C₄alkoxy or a fused benzo ring, which benzo is optionally substituted with halogen;

R¹⁰ is hydrogen or C₁-C₄alkyl or R⁷ and R¹⁰, taken in combination, form a —(CH₂)ₚ— group or a —O—(CH₂)_{q}— group, wherein p is 2, 3, or 4 and q is 1 or 2;

R¹¹ is hydrogen or C₁-C₄alkyl;

R¹² is hydrogen, halogen, hydroxy, C₁-C₄alkyl or C₁-C₄alkoxy;

R¹³ is hydrogen or halogen;

R¹⁴ is hydrogen or halogen;

R¹⁵ is hydrogen, C₁-C₄alkyl, or NHC(O)R¹⁶; and

R¹⁶ is C₁-C₄alkyl or cyclopropyl each of which is optionally substituted by phenyl; or

R⁷ and R¹⁶ taken in combination for a divalent C₂-C₃ alkylene group.

In a second embodiment, the invention provides compounds of Formula Ia and pharmaceutically acceptable salts thereof, which modulate the alternative pathway of the complement system. Compounds of Formula Ia are represented by the structure:

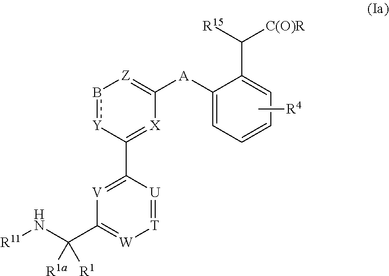

or a salt thereof, wherein

A is —C(O)NH—, —C≡C—, —CH₂CH₂—, S(O)₂N(H)—, or —CHR¹⁰O—, wherein the carbon or sulfur is attached to the ring comprising X, Y and Z; or

A is —NHCH₂— or —OCH₂—, wherein the nitrogen or oxygen is attached to the ring comprising X, Y and Z; or

R is hydroxy, amino or C₁-C₄alkoxy;

R¹ is hydrogen, phenyl, C₃-C₆cycloalkyl, amido, halo C₁-C₄alkyl or C₁-C₄alkyl optionally substituted by hydroxy, C₃-C₆ cycloalkyl, C₁-C₄alkoxy or cyano;

R^{1a} is hydrogen or C₁-C₄alkyl, or CR¹R^{1a} taken in combination form a carbonyl (C═O), imine (C═NH) or a 3-6 member cycloalkyl;

R^{1a} is absent and CR¹ and R¹¹, taken in combination, form a saturated, unsaturated or aromatic 4, 5 or 6 member azacycle;

T is CR² or N;

U is CR¹⁴ or N;

V is CR¹² or N;

W is CR¹³ or N, wherein 0, 1, or 2 of T, U, V and W are N; or

V is N, W is S, T is absent and U is CR¹⁴;

B is CR³ or N;

X is CR⁶ or N;

Y is CR⁵ or N;

Z is CR⁷ or N, wherein 0 or 1 of B, X, Y and Z are nitrogen; or

X is N, B is CR³ and one of Y or Z is S or N(H) and other of Y or Z is absent;

R² and R¹⁴ are independently selected from the group consisting of hydrogen and halogen;

R³ is hydrogen, halogen, hydroxy, cyano, amino, NHR⁸, N(R⁸)₂, —C(O)NHR⁸, OR⁹, C₁-C₄alkyl, haloC₁-C₄alkyl, C₃-C₆cycloalkyl, phenyl, heterocycloalkyl having 4 to 7 ring atoms and 1, 2, or 3 ring heteroatoms independently selected from N, O and S, and heteroaryl having 5, 6, 9 or 10 ring atoms and 1, 2, or 3 ring heteroatoms independently selected from N, O and S, wherein each heterocycloalkyl, heteroaryl, phenyl is optionally substituted with 0, 1, 2, or 3 substituents independently selected from C₁-C₄alkyl, C₁-C₄alkoxy, halogen, or C₃-C₆cycloalkyl, wherein each heterocycloalkyl or heteroaryl is optionally further substituted by 0 or 1 phenyl groups and wherein each alkyl, haloalkyl and cycloalkyl group is optionally substituted with 0, 1, or 2 substituents independently selected from the group consisting of hydroxy, C₃-C₆cycloalkyl, amino, NHR⁸, N(R⁸)₂, OR⁹ and heterocycloalkyl having 4 to 7 ring atoms and 1, 2, or 3 ring heteroatoms independently selected from N, O and S;
R⁴ represents 0, 1, or 2 substituents independently selected from halogen, C₁-C₄alkyl, C₁-C₄alkoxy, and hydroxyC₁-C₄alkyl;

R⁵ is hydrogen, halogen, C₁-C₄alkyl or C₁-C₄alkoxy;

R⁶ is hydrogen, halogen, C₁-C₄alkyl or C₁-C₄alkoxy;

R⁷ is hydrogen, halogen, C₁-C₄alkyl, C₁-C₄alkoxy, haloC₁-C₄alkyl or haloC₁-C₄alkoxy, or

R⁷ is phenyl or a 5 or 6 member heteroaryl having 1, 2, or 3 ring heteroatoms selected from N, O or S; each of which is optionally substituted by 0, 1, or 2 substituents selected from C₁-C₄alkyl, aminoC₁-C₄alkyl, hydroxyC₁-C₄alkyl, halogen, C₁-C₄alkoxy, hydroxy, amino or mono- or di-C₁-C₄alkylamino; or

R³ and either R⁵ or R⁷ taken in combination form a —O(CH₂)ₙO— group wherein n is 1 or 2; or

R³ and R⁷ taken in combination with the atoms to which they are attached form a 5 or 6 member aromatic heterocycle having 1 or 2 ring heteroatoms selected from N, O or S and which is optionally substituted with C₁-C₄alkyl, C(O)C₁-C₄alkyl, C(O)NH2, C(O)NHC₁-C₄alkyl, C(O)N(C₁-C₄alkyl)₂, S(O)₂C₁-C₄alkyl, S(O)₂C3-C₆cycloalkyl, optionally substituted S(O)₂phenyl, where the phenyl is optionally substituted with 0, 1, or 2 C₁-C₄alkyl, or C₁-C₄alkoxy or C₁-C₄alkoxy;

R⁸ is independently selected at each occurrence from the group consisting of hydrogen, C₁-C₄alkyl, haloC₁-C₄alkyl, C₃-C₆cycloalkyl, benzyl, C₁-C₄alkanoyl, benzoyl, phenyl, 4 to 6 member heterocycloalkyl, heteroaryl, wherein C₁-C₄alkyl is optionally substituted with C₁-C₄alkoxy, C₃-C₆cycloalkyl, cyano, 4 to 6 member heterocycloalkyl or heteroaryl, wherein phenyl or benzyl are optionally substituted with 0, 1, or 2 substitutents selected from C₁-C₄alkyl, CH₂CO₂H, C₃-C₆cycloalkyl or C₁-C₄alkoxy, and wherein each heterocycloalkyl or heteroaryl are optionally substituted by 0, 1, or 2 substituents independently selected from C₁-C₄alkyl, CO₂ C₁-C₄alkyl, C(O)NH C₁-C₄alkyl, C₃-C₆cycloalkyl, or C₁-C₄alkoxy;

R⁹ is independently selected at each occurrence from the group consisting of hydrogen, C₁-C₄alkyl, haloC₁-C₄alkyl, C₃-C₆cycloalkyl, benzyl, benzoyl, phenyl, 4 to 6 member heterocycloalkyl, heteroaryl, wherein C₁-C₄alkyl is optionally substituted with C₁-C₄alkoxy, C₃-C₆cycloalkyl, cyano, 4 to 6 member heterocycloalkyl or heteroaryl, wherein phenyl or benzyl are optionally substituted with 0, 1, or 2 substitutents selected from C₁-C₄alkyl, CH₂CO₂H, C₃-C₆cycloalkyl or C₁-C₄alkoxy, and wherein each heterocycloalkyl or heteroaryl are optionally substituted by 0, 1, or 2 substituents independently selected from C₁-C₄alkyl, CO₂ C₁-C₄alkyl, C(O)NH C₁-C₄alkyl, C₃-C₆cycloalkyl, or C₁-C₄alkoxy;

R¹⁰ is hydrogen or C₁-C₄alkyl or R⁷ and R¹⁰, taken in combination, form a —(CH₂)ₚ— group or a —O—(CH₂)_{q}— group, wherein p is 2, 3, or 4 and q is 1 or 2;

R¹¹ is hydrogen or C₁-C₄alkyl;

R¹² is hydrogen, halogen, hydroxy, C₁-C₄alkyl or C₁-C₄alkoxy;

R¹³ is hydrogen or halogen;

R¹⁴ is hydrogen or halogen; and

R¹⁵ is hydrogen or C₁-C₄alkyl.

In a third embodiment, the invention provides compounds, salts thereof and tautomers thereof of the first or second embodiment, which compound is represented by formula (II)

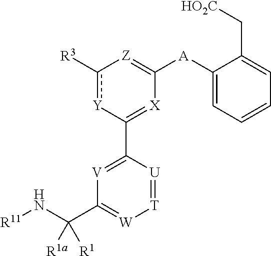

or a salt thereof.

In a fourth embodiment, the invention provides compounds, salts thereof and tautomers thereof of any one of the first to third embodiment, in which the compound is represented by formula (III),

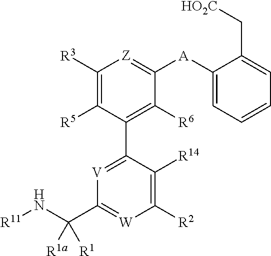

or a salt thereof, wherein

V is CR¹², W is CR¹³ and Z is CR⁷; or

V is N, W is CR¹³ and Z is R⁷; or

V is CR¹², W is N and Z is CR⁷; or

V is CR¹², W is CR¹³ and Z is N.

In a fifth embodiment, compounds of any one of embodiments one to four are provided in which V is CR¹², W is CR¹³ and Z is CR⁷.

In a sixth embodiment, compounds of any one of embodiments one to four are provided in which V is N, W is CR¹³ and Z is CR⁷; or

In a seventh embodiment, compounds of any one of embodiments one to four are provided in which V is CR¹², W is N and Z is CR⁷.

In a eighth embodiment, compounds of any one of embodiments one to four are provided, wherein V is CR¹², W is CR¹³ and Z is N.

In a ninth embodiment, compounds of any one of embodiments one to eight are provided in which A is CH₂O or —C(O)NH—, wherein the carbon is attached to the ring comprising X, Y and Z.

In a tenth embodiment, compounds of any one of embodiments one to nine are provided in which A is CH₂O, wherein the carbon is attached to the ring comprising X, Y and Z.

In an eleventh embodiment, compounds of any one of embodiments one to ten are provided in which R¹ is hydrogen, C₁-C₄alkyl, hydroxyC₁-C₄alkyl or fluoro C₁-C₄alkyl; and

R^{1a} is hydrogen.

In a twelfth embodiment, compounds of any one of embodiments one to eleven are provided in which R¹ is hydrogen, methyl, hydroxymethyl, or fluoromethyl; and

R^{1a} is hydrogen.

In a thirteenth embodiment, compounds of any one of embodiments one to twelve are provided in which R¹¹ is hydrogen.

In a fourteenth embodiment, compounds of any one of embodiments one to thirteen are provided in which R³ is hydrogen or halogen.

In a fifteenth embodiment, compounds of any one of embodiments one to fourteen are provided in which R³ is hydrogen or R³ is C₁-C₄alkoxy or C₁-C₄alkylamine, each of which is optionally substituted by C₃-C₆cycloalkyl or a 4 to 6 member saturated heterocycle, which heterocycle has 1 or 2 ring heteroatoms selected from N, O or S.

In a sixteenth embodiment, compounds of any one of embodiments one to fifteen are provided in which R³ is hydrogen or R³ is methoxy, ethoxy, methylamino or ethylamino, each of which is optionally substituted by trifluoromethyl, cyclopropyl, cyclobutyl, cyclopentyl, cyclohexyl, tetrahydrofuranyl, tetrahydropyranyl, dioxanyl, or a saturated azacycle having 4 to 6 ring atoms and 1 or 2 ring nitrogen atoms. In certain aspects of the fifteenth embodiment, compounds are provided in which R³ is hydrogen or R³ is, methoxy or methylamino, each of which is optionally substituted by cyclopropyl, cyclobutyl, cyclopentyl, cyclohexyl, tetrahydrofuranyl, tetrahydropyranyl, dioxanyl, or a saturated azacycle having 4 to 6 ring atoms and 1 or 2 ring nitrogen atoms;

In a seventeenth embodiment, compounds of any one of embodiments one to sixteen are provided in which R⁵ is hydrogen or halogen.

In an eighteenth embodiment, compounds of any one of embodiments one to seventeen are provided in which R⁶ is hydrogen or halogen.

In a ninteenth embodiment, compounds of any one of embodiments one to seven or nine to eighteen are provided in which Z is CR⁷ and R⁷ is hydrogen or halogen.

In a twentieth embodiment, compounds of any one of embodiments one to nineteen are provided in which R⁵, R⁶ and R⁷ are hydrogen.

In a twenty-first embodiment, compounds of any one of embodiments one to seven and nine to eighteen are provided in which Z is CR⁷; and R⁷ and R³ together form —N(H)—N═CH— where the carbon is attached at Z.

In a twenty second embodiment, compounds of any one of embodiments one to twenty-first are provided in which R¹⁴ is hydrogen or halogen.

In a twenty third embodiment, compounds of any one of embodiments one to five and nine to twenty-one-are provided in which V is CR¹² and R¹² is hydrogen or fluorine. In certain compounds for the twenty third embodiment, R¹² is fluorine.

In a twenty fourth embodiment, compounds of any one of embodiments one to twenty three are provided in which R², R⁵, R⁶, R¹⁰, R¹², R¹³ and R¹⁴ are hydrogen.

In a twenty fifth embodiment, compounds of embodiments one or two are provided, which compounds, and salts thereof, are selected from the group consisting of
methyl 2-(2-(3′-(aminomethyl)-[1,1′-biphenyl]-3-ylcarboxamido)phenyl)acetate;
2-(2-(3′-(aminomethyl)-[1,1′-biphenyl]-3-ylcarboxamido)phenyl)acetic acid;
N-(2-(2-amino-2-oxoethyl)phenyl)-3′-(aminomethyl)-[1,1′-biphenyl]-3-carboxamide;
methyl 2-(2-(5′-(aminomethyl)-2′-fluoro-[1,1′-biphenyl]-3-ylcarboxamido)phenyl)acetate;
2-(2-(5′-(aminomethyl)-2′-fluoro-[1,1′-biphenyl]-3-ylcarboxamido)phenyl)acetic acid;
2-(2-(5-acetamido-3′-(aminomethyl)-[1,1′-biphenyl]-3-ylcarboxamido)phenyl)acetic acid;
2-(2-(3′-(aminomethyl)-5′-fluoro-[1,1′-biphenyl]-3-ylcarboxamido)phenyl)acetic acid;
2-(2-(3′-(aminomethyl)-5-(methylcarbamoyl)-[1,1′-biphenyl]-3-ylcarboxamido)phenyl)acetic acid;
methyl 2-(2-(3′-(aminomethyl)-6-methoxy-[1,1′-biphenyl]-3-ylcarboxamido)phenyl)acetate;
2-(2-(3′-(aminomethyl)-6-methoxy-[1,1′-biphenyl]-3-ylcarboxamido)phenyl)acetic acid;
methyl 2-(2-(3′-(aminomethyl)-5′-fluoro-6-methoxy-[1,1′-biphenyl]-3-ylcarboxamido)phenyl)acetate;
2-(2-(3′-carbamimidoyl-[1,1′-biphenyl]-3-ylcarboxamido)phenyl)acetic acid;
2-(2-(3′-(aminomethyl)-5′-fluoro-5-(isoxazol-4-yl)-[1,1′-biphenyl]-3-ylcarboxamido)phenyl)acetic acid;
2-(2-(3′-(aminomethyl)-5-bromo-5′-fluoro-[1,1′-biphenyl]-3-ylcarboxamido)phenyl)acetic acid;
2-(2-(3′-(aminomethyl)-5′-fluoro-5-(phenylamino)-[1,1′-biphenyl]-3-ylcarboxamido)phenyl)acetic acid;
2-(2-(3′-(aminomethyl)-5′-fluoro-5-((5-methoxy-2-methylphenyl)amino)-[1,1′-biphenyl]-3-ylcarboxamido)phenyl)acetic acid;
2-(2-(3′-(aminomethyl)-5′-fluoro-5-((pyridin-4-ylmethyl)amino)-[1,1′-biphenyl]-3-ylcarboxamido)phenyl)acetic acid;
2-(2-(3′-(aminomethyl)-5′-fluoro-5-(((1-methyl-1H-pyrazol-3-yl)methyl)amino)-[1,1′-biphenyl]-3-ylcarboxamido)phenyl)acetic acid;
(R)-2-(2-(3′-(aminomethyl)-5′-fluoro-5-(((tetrahydrofuran-2-yl)methyl)amino)-[1,1′-biphenyl]-3-ylcarboxamido)phenyl)acetic acid;
(S)-2-(2-(3′-(aminomethyl)-5′-fluoro-5-((tetrahydrofuran-2-yl)methoxy)-[1,1′-biphenyl]-3-ylcarboxamido)phenyl)acetic acid;
2-(2-((3′-(aminomethyl)-[1,1′-biphenyl]-3-yl)ethynyl)phenyl)acetic acid;
2-(2-(2-(3′-(aminomethyl)-[1,1′-biphenyl]-3-yl)ethyl)phenyl)acetic acid;
2-(2-(3′-(aminomethyl)-[1,1′-biphenyl]-3-ylcarboxamido)-4-chlorophenyl)acetic acid;
2-(2-(3′-(aminomethyl)-5′-fluoro-[1,1′-biphenyl]-3-ylcarboxamido)-5-bromophenyl)acetic acid;
(±)-2-(2-(3′-(aminomethyl)-5′-fluoro-[1,1′-biphenyl]-3-ylcarboxamido)phenyl)propanoic acid;
2-(2-(6-(3-(aminomethyl)-5-fluorophenyl)picolinamido)phenyl)acetic acid;
2-(2-(3′-(aminomethyl)-5′-fluoro-[1,1′-biphenyl]-3-ylcarboxamido)-4-bromophenyl)acetic acid;
2-(2-(3′-(aminomethyl)-5′-fluoro-[1,1′-biphenyl]-3-ylcarboxamido)-4-methylphenyl)acetic acid;
2-(2-(3′-(aminomethyl)-5′-fluoro-[1,1′-biphenyl]-3-ylcarboxamido)-3-methylphenyl)acetic acid;
2-(2-(3′-(aminomethyl)-5′-fluoro-[1,1′-biphenyl]-3-ylcarboxamido)-3-fluorophenyl)acetic acid;
2-(2-(6-(3-(aminomethyl)-5-fluorophenyl)picolinamido)-3-bromophenyl)acetic acid;
2-(2-(3′-(aminomethyl)-5′-fluoro-[1,1′-biphenyl]-3-ylcarboxamido)-6-chlorophenyl)acetic acid;
2-(2-(3′-(aminomethyl)-5′-fluoro-[1,1′-biphenyl]-3-ylcarboxamido)-6-bromophenyl)acetic acid;
2-(2-(3′-(aminomethyl)-5′-fluoro-[1,1′-biphenyl]-3-ylcarboxamido)-6-methylphenyl)acetic acid;
2-(2-(3′-(aminomethyl)-5′-fluoro-[1,1′-biphenyl]-3-ylcarboxamido)-4-fluorophenyl)acetic acid;
2-(2-(3′-(aminomethyl)-5′-fluoro-[1,1′-biphenyl]-3-ylcarboxamido)-5-fluorophenyl)acetic acid;
2-(2-(3′-(aminomethyl)-5′-fluoro-[1,1′-biphenyl]-3-ylcarboxamido)-3-methoxyphenyl)acetic acid;
2-(2-(6-(3-(aminomethyl)-5-fluorophenyl)picolinamido)-5-ethylphenyl)acetic acid;
2-(2-(6-(3-(aminomethyl)-5-fluorophenyl)picolinamido)-4-methylphenyl)acetic acid;
2-(2-(6-(3-(aminomethyl)-5-fluorophenyl)picolinamido)-3-chlorophenyl)acetic acid;
2-(2-(6-(3-(aminomethyl)-5-fluorophenyl)picolinamido)-4-ethylphenyl)acetic acid;
2-(2-(6-(3-(aminomethyl)-5-fluorophenyl)picolinamido)-5-chlorophenyl)acetic acid;
2-(2-(6-(3-(aminomethyl)-5-fluorophenyl)picolinamido)-4-methoxyphenyl)acetic acid;
2-(2-(3′-(aminomethyl)-5′-fluoro-[1,1′-biphenyl]-3-ylcarboxamido)-5-methylphenyl)acetic acid;
2-(2-(3′-(aminomethyl)-5′-fluoro-[1,1′-biphenyl]-3-ylcarboxamido)-5-ethylphenyl)acetic acid;
2-(2-(3′-(aminomethyl)-5′-fluoro-[1,1′-biphenyl]-3-ylcarboxamido)-5-(1-hydroxyethyl)phenyl)acetic acid;
(S)-2-(2-(3′-(aminomethyl)-5′-fluoro-5-(((tetrahydrofuran-2-yl)methyl)amino)-[1,1′-biphenyl]-3-ylcarboxamido)-5-ethylphenyl)acetic acid;
2-(2-(4-(3-(aminomethyl)phenyl)thiazole-2-carboxamido)phenyl)acetic acid;
2-(2-(2-(3-(aminomethyl)phenyl)thiazole-4-carboxamido)phenyl)acetic acid;
2-(2-(4-(3-(aminomethyl)-5-fluorophenyl)thiazole-2-carboxamido)phenyl)acetic acid;
2-(2-(2-(3-(aminomethyl)-5-fluorophenyl)thiazole-4-carboxamido)phenyl)acetic acid;
2-(2-(2-(3-(aminomethyl)-5-fluorophenyl)-5-chlorothiazole-4-carboxamido)phenyl)acetic acid;
2-(2-(2-(3-(aminomethyl)-5-fluorophenyl)-5-(methylamino)thiazole-4-carboxamido)phenyl)acetic acid;
2-(2-(2-(3-(aminomethyl)-5-fluorophenyl)-5-morpholinothiazole-4-carboxamido)phenyl)acetic acid;
(S)-2-(2-(2-(3-(aminomethyl)-5-fluorophenyl)-5-(((tetrahydrofuran-2-yl)methyl)amino)thiazole-4-carboxamido)phenyl)acetic acid;
2-(2-(2-(3-(aminomethyl)-5-fluorophenyl)-5-(methylamino)thiazole-4-carboxamido)-4-methylphenyl)acetic acid;
2-(2-(2-(3-(aminomethyl)-5-fluorophenyl)-5-cyclopropylthiazole-4-carboxamido)phenyl)acetic acid;
2-(2-(2-(3-(aminomethyl)-5-fluorophenyl)-5-ethylthiazole-4-carboxamido)phenyl)acetic acid;
2-(2-(3′-(aminomethyl)-2′-fluoro-[1,1′-biphenyl]-3-ylcarboxamido)phenyl)acetic acid;
2-(2-(2-(3-(aminomethyl)phenyl)isonicotinamido)phenyl)acetic acid;
2-(2-(4-(3-(aminomethyl)phenyl)picolinamido)phenyl)acetic acid;
2-(2-(5-(3-(aminomethyl)phenyl)nicotinamido)phenyl)acetic acid;
2-(2-(6-(3-(aminomethyl)phenyl)-4-chloropicolinamido)phenyl)acetic acid;
2-(2-(4,6-bis(3-(aminomethyl)phenyl)picolinamido)phenyl)acetic acid;
2-(2-(2-(3-(aminomethyl)phenyl)-1H-imidazole-4-carboxamido)phenyl)acetic acid;
2-(2-(6-(3-(aminomethyl)phenyl)-4-morpholinopicolinamido)phenyl)acetic acid;
(S)-2-(2-(6-(3-(aminomethyl)phenyl)-4-(((tetrahydrofuran-2-yl)methyl)amino)picolinamido)phenyl)acetic acid;
2-(2-(6-(3-(aminomethyl)phenyl)-4-(methylamino)picolinamido)phenyl)acetic acid;
2-(2-(4-(3-(aminomethyl)phenyl)-1H-imidazole-2-carboxamido)phenyl)acetic acid;
2-(2-(3′-(aminomethyl)-[1,1′-biphenyl]-3-ylsulfonamido)phenyl)acetic acid;
2-(2-(3′-(aminomethyl)-5′-fluoro-[1,1′-biphenyl]-3-ylsulfonamido)phenyl)acetic acid;
2-(2-(6-(3-(aminomethyl)phenyl)-4-(cyclopropylamino)picolinamido)phenyl)acetic acid;
2-(2-(6-(3-(aminomethyl)phenyl)-4-(4-phenylpiperazin-1-yl)picolinamido)phenyl)acetic acid;
2-(2-(6-(3-(aminomethyl)phenyl)-4-phenylpicolinamido)phenyl)acetic acid;
2-(2-(6-(3-(aminomethyl)phenyl)-4-(benzylamino)picolinamido)phenyl)acetic acid;
2-(2-(6-(3-(aminomethyl)phenyl)-4-((2,2,2-trifluoroethyl)amino)picolinamido)phenyl)acetic acid;
2-(2-(6-(3-(aminomethyl)phenyl)-4-hydroxypicolinamido)phenyl)acetic acid;
2-(2-(6-(3-(aminomethyl)phenyl)-3-chloropicolinamido)phenyl)acetic acid;
2-(2-(3,6-bis(3-(aminomethyl)phenyl)picolinamido)phenyl)acetic acid;
2-(2-(6-(3-(aminomethyl)phenyl)picolinamido)phenyl)acetic acid;
2-(2-(((3′-(aminomethyl)-[1,1′-biphenyl]-3-yl)amino)methyl)phenyl)acetic acid;
2-(2-(3′-(aminomethyl)-5-hydroxy-[1,1′-biphenyl]-3-ylcarboxamido)phenyl)acetic acid;
2-(2-(3′-(aminomethyl)-5′-fluoro-5-hydroxy-[1,1′-biphenyl]-3-ylcarboxamido)phenyl)acetic acid;
2-(2-(3′-(aminomethyl)-5-(pyrimidin-2-ylmethoxy)-[1,1′-biphenyl]-3-ylcarboxamido)phenyl)acetic acid;
2-(2-(3′-(aminomethyl)-5-(pyridin-4-ylmethoxy)-[1,1′-biphenyl]-3-ylcarboxamido)phenyl)acetic acid;
(±)-2-(2-(3′-(aminomethyl)-5-((tetrahydrofuran-2-yl)methoxy)-[1,1′-biphenyl]-3-ylcarboxamido)phenyl)acetic acid;
(±)-2-(2-(3′-(aminomethyl)-5-(1-cyanoethoxy)-[1,1′-biphenyl]-3-ylcarboxamido)phenyl)acetic acid;
2-(2-(3′-(aminomethyl)-5′-fluoro-5-(pyridin-4-ylmethoxy)-[1,1′-biphenyl]-3-ylcarboxamido)phenyl)acetic acid;
2-(2-(3′-(aminomethyl)-5-((1-methyl-1H-pyrazol-3-yl)methoxy)-[1,1′-biphenyl]-3-ylcarboxamido)phenyl)acetic acid;
2-(2-(5-acetamido-3′-(aminomethyl)-5′-fluoro-[1,1′-biphenyl]-3-ylcarboxamido)phenyl)acetic acid;
ethyl 2-(2-(5-acetamido-3′-(aminomethyl)-5′-fluoro-[1,1′-biphenyl]-3-ylcarboxamido)phenyl)acetate;
2-(2-(3′-(aminomethyl)-5′-fluoro-5-(pyrimidin-2-ylmethoxy)-[1,1′-biphenyl]-3-ylcarboxamido)phenyl)acetic acid;
2-(2-(3′-(aminomethyl)-5′-fluoro-5-((1-methyl-1H-pyrazol-3-yl)methoxy)-[1,1′-biphenyl]-3-ylcarboxamido)phenyl)acetic acid;
2-(2-(3′-(aminomethyl)-5-((2-(methyl carbamoyl)pyridin-4-yl)methoxy)-[1,1′-biphenyl]-3-ylcarboxamido)phenyl)acetic acid;
2-(2-(3′-(aminomethyl)-5′-fluoro-5-((2-(methylcarbamoyl)pyridin-4-yl)methoxy)-[1,1′-biphenyl]-3-ylcarboxamido)phenyl)acetic acid;
2-(2-(3′-(aminomethyl)-5-(2-methoxyethoxy)-[1,1′-biphenyl]-3-ylcarboxamido)phenyl)acetic acid;
2-(2-(3′-(aminomethyl)-5′-fluoro-5-(2-methoxyethoxy)-[1,1′-biphenyl]-3-ylcarboxamido)phenyl)acetic acid;
(R)-2-(2-(3′-(aminomethyl)-5-((tetrahydrofuran-2-yl)methoxy)-[1,1′-biphenyl]-3-ylcarboxamido)phenyl)acetic acid;
(S)-2-(2-((3′-(1-amino-2-hydroxyethyl)-[1,1′-biphenyl]-3-yl)methoxy)-3-fluorophenyl)acetic acid;
(R)-2-(2-(3′-(aminomethyl)-5′-fluoro-5-((tetrahydrofuran-2-yl)methoxy)-[1,1′-biphenyl]-3-ylcarboxamido)phenyl)acetic acid;
2-(2-(3′-(aminomethyl)-5′-fluoro-5-morpholino-[1,1′-biphenyl]-3-ylcarboxamido)phenyl)acetic acid;
2-(2-(3′-(aminomethyl)-5-((3-cyclopropyl-1-methyl-1H-pyrazol-5-yl)amino)-5′-fluoro-[1,1′-biphenyl]-3-ylcarboxamido)phenyl)acetic acid;
2-(2-(3′-(aminomethyl)-5′-fluoro-5-(((tetrahydrofuran-3-yl)methyl)amino)-[1,1′-biphenyl]-3-ylcarboxamido)phenyl)acetic acid;
2-(2-(3′-(aminomethyl)-5′-fluoro-5-((tetrahydrofuran-3-yl)amino)-[1,1′-biphenyl]-3-ylcarboxamido)phenyl)acetic acid;
2-(2-(3′-(aminomethyl)-5′-fluoro-5-((1-methyl-1H-pyrazol-3-yl)amino)-[1,1′-biphenyl]-3-ylcarboxamido)phenyl)acetic acid;
2-(2-(3′-(aminomethyl)-5′-fluoro-5-((tetrahydro-2H-pyran-4-yl)amino)-[1,1′-biphenyl]-3-ylcarboxamido)phenyl)acetic acid;
2-(2-(3′-(aminomethyl)-5′-fluoro-5-(((tetrahydro-2H-pyran-4-yl)methyl)amino)-[1,1′-biphenyl]-3-ylcarboxamido)phenyl)acetic acid;
(S)-2-(2-(3′-(aminomethyl)-5′-fluoro-5-(((tetrahydrofuran-2-yl)methyl)amino)-[1,1′-biphenyl]-3-ylcarboxamido)phenyl)acetic acid;
2-(2-(5-((1H-pyrazol-5-yl)amino)-3′-(aminomethyl)-5′-fluoro-[1,1′-biphenyl]-3-ylcarboxamido)phenyl)acetic acid;
2-(2-(3′-(aminomethyl)-5′-fluoro-5-((3-methyl-1H-pyrazol-5-yl)amino)-[1,1′-biphenyl]-3-ylcarboxamido)phenyl)acetic acid;
2-(2-(3′-(aminomethyl)-5-((1,3-dimethyl-1H-pyrazol-5-yl)amino)-5′-fluoro-[1,1′-biphenyl]-3-ylcarboxamido)phenyl)acetic acid;
2-(2-(3′-((R)-1-aminoethyl)-5-(((S)-tetrahydrofuran-2-yl)methoxy)-[1,1′-biphenyl]-3-ylcarboxamido)phenyl)acetic acid;
2-(2-(3′-((S)-1-amino-2-hydroxyethyl)-5-(((S)-tetrahydrofuran-2-yl)methoxy)-[1,1′-biphenyl]-3-ylcarboxamido)phenyl)acetic acid;
2-(2-(3,3″-bis((S)-1-amino-2-hydroxyethyl)-[1,1′:3′,1″-terphenyl]-5′-ylcarboxamido)phenyl)acetic acid;
(S)-2-(2-(3′-(1-amino-2-hydroxyethyl)-5-((2-methoxyethyl)amino)-[1,1′-biphenyl]-3-ylcarboxamido)phenyl)acetic acid;
(S)-2-(2-((3′-(1-amino-2-hydroxyethyl)-5-((2-methoxyethyl)amino)-[1,1′-biphenyl]-3-yl)methoxy)phenyl)acetic acid;
(R)-Ethyl 2-(2-(3′-(aminomethyl)-5′-fluoro-5-(((tetrahydrofuran-2-yl)methyl)amino)-[1,1′-biphenyl]-3-ylcarboxamido)phenyl)acetate;
(±)-2-(2-(3′-(1-amino-2-hydroxyethyl)-[1,1′-biphenyl]-3-ylcarboxamido)phenyl)acetic acid;
(±)-2-(2-(3′-(1-amino-3-hydroxypropyl)-[1,1′-biphenyl]-3-ylcarboxamido)phenyl)acetic acid;
(R)-2-(2-(3′-(1-amino-3-hydroxypropyl)-[1,1′-biphenyl]-3-ylcarboxamido)phenyl)acetic acid;
(S)-2-(2-(3′-(1-amino-3-hydroxypropyl)-[1,1′-biphenyl]-3-ylcarboxamido)phenyl)acetic acid;
(R)-2-(2-(3′-(1-aminoethyl)-[1,1′-biphenyl]-3-ylcarboxamido)phenyl)acetic acid;
2-(2-(3′-(aminomethyl)-2-methoxy-[1,1′-biphenyl]-3-ylcarboxamido)phenyl)acetic acid;
2-(2-(3′-(1-aminocyclopropyl)-[1,1′-biphenyl]-3-ylcarboxamido)phenyl)acetic acid;
2-(2-(3′-(aminomethyl)-5′-fluoro-2-methoxy-[1,1′-biphenyl]-3-ylcarboxamido)phenyl)acetic acid;
(R)-2-(2-((3′-(1-amino-3-hydroxypropyl)-5-((cyclopropylmethyl)amino)-[1,1′-biphenyl]-3-yl)methoxy)phenyl)acetic acid;
3′-(aminomethyl)-5-((2-(carboxymethyl)phenyl)carbamoyl)-5′-fluoro-[1,1′-biphenyl]-3-carboxylic acid;
2-(2-(3′-(aminomethyl)-5′-fluoro-5-propionamido-[1,1′-biphenyl]-3-ylcarboxamido)phenyl)acetic acid;
2-(2-(3′-(aminomethyl)-5′-fluoro-5-isobutyramido-[1,1′-biphenyl]-3-ylcarboxamido)phenyl)acetic acid;
2-(2-(3′-(aminomethyl)-5′-fluoro-5-(3-methylbutanamido)-[1,1′-biphenyl]-3-ylcarboxamido)phenyl)acetic acid;
2-(2-(((6-(3-(aminomethyl)phenyl)pyridin-2-yl)oxy)methyl)phenyl)acetic acid;
2-(2-(((3′-(aminomethyl)-[1,1′-biphenyl]-3-yl)oxy)methyl)phenyl)acetic acid;
(S)-2-(2-((3′-(1-amino-2-hydroxyethyl)-[1,1′-biphenyl]-3-yl)methoxy)phenyl)acetic acid;
2-(2-((3′-(aminomethyl)-5-chloro-[1,1′-biphenyl]-3-yl)methoxy)phenyl)acetic acid;
2-(2-((3′-(aminomethyl)-5-cyclopropyl-[1,1′-biphenyl]-3-yl)methoxy)phenyl)acetic acid;
2-(2-((3′-(aminomethyl)-5-ethyl-[1,1′-biphenyl]-3-yl)methoxy)phenyl)acetic acid;
2-(2-((3′-(aminomethyl)-5-(2-hydroxyethyl)-[1,1′-biphenyl]-3-yl)methoxy)phenyl)acetic acid;
(±)-2-(2-((3′-(aminomethyl)-5-(1-hydroxyethyl)-[1,1′-biphenyl]-3-yl)methoxy)phenyl)acetic acid;
2-(2-((3′-(aminomethyl)-5-(2-cyclopropylethyl)-[1,1′-biphenyl]-3-yl)methoxy)phenyl)acetic acid;
(S)-2-(2-((3′-(1-amino-2-hydroxyethyl)-5-chloro-[1,1′-biphenyl]-3-yl)methoxy)phenyl)acetic acid;
(S)-2-(2-((3′-(1-amino-2-hydroxyethyl)-5-cyclopropyl-[1,1′-biphenyl]-3-yl)methoxy)phenyl)acetic acid;
(S)-2-(2-((3′-(1-amino-2-hydroxyethyl)-5-ethyl-[1,1′-biphenyl]-3-yl)methoxy)phenyl)acetic acid;
(S)-2-(2-((3′-(1-amino-2-hydroxyethyl)-5-bromo-[1,1′-biphenyl]-3-yl)methoxy)phenyl)acetic acid;
(S)-tert-butyl 2-(2-((3′-(1-((tert-butoxycarbonyl)amino)-2-hydroxyethyl)-5-methyl-[1,1′-biphenyl]-3-yl)methoxy)phenyl)acetate;
(S)-2-(2-((3′-(1-amino-2-hydroxyethyl)-5-methyl-[1,1′-biphenyl]-3-yl)methoxy)phenyl)acetic acid;
2-(2-((3′-(aminomethyl)-5-cyano-[1,1′-biphenyl]-3-yl)methoxy)phenyl)acetic acid;
(S)-2-(2-((3′-(1-amino-2-hydroxyethyl)-5-(trifluoromethyl)-[1,1′-biphenyl]-3-yl)methoxy)phenyl)acetic acid;
(S)-2-(2-((3′-(1-amino-2-hydroxyethyl)-5-fluoro-[1,1′-biphenyl]-3-yl)methoxy)phenyl)acetic acid;
(S)-2-(2-((3′-(1-amino-2-hydroxyethyl)-5-ethoxy-[1,1′-biphenyl]-3-yl)methoxy)phenyl)acetic acid;
(S)-2-(2-((3′-(1-amino-2-hydroxyethyl)-5-(2,2,2-trifluoroethoxy)-[1,1′-biphenyl]-3-yl)methoxy)phenyl)acetic acid;
(R)-2-(2-((3′-(1-aminoethyl)-5-(hydroxymethyl)-[1,1′-biphenyl]-3-yl)methoxy)phenyl)acetic acid;
(S)-2-(2-((3′-(1-amino-2-hydroxyethyl)-5-(hydroxymethyl)-[1,1′-biphenyl]-3-yl)methoxy)phenyl)acetic acid;
2,2′-((((3′-(aminomethyl)-[1,1′-biphenyl]-3,5-diyl)bis(methylene))bis(oxy))bis(2,1-phenylene))diacetic acid;
(S)-2-(2-((3′-(1-amino-2-hydroxyethyl)-5-(methoxymethyl)-[1,1′-biphenyl]-3-yl)methoxy)phenyl)acetic acid;
2-(2-((3′-((S)-1-amino-2-hydroxyethyl)-5-(1-hydroxyethyl)-[1,1′-biphenyl]-3-yl)methoxy)phenyl)acetic acid (mixture of diastereomers);
2-(2-((3′-((S)-1-amino-2-hydroxyethyl)-5-(1-hydroxyethyl)-[1,1′-biphenyl]-3-yl)methoxy)phenyl)acetic acid (first diastareomer);
2-(2-((3′-((S)-1-amino-2-hydroxyethyl)-5-(1-hydroxyethyl)-[1,1′-biphenyl]-3-yl)methoxy)phenyl)acetic acid (second diastareomer);
2-(2-((3′-((R)-1-aminoethyl)-5-(1-hydroxyethyl)-[1,1′-biphenyl]-3-yl)methoxy)phenyl)acetic acid (mixture of diastereomers);
2-(2-((3′-((S)-1-amino-2-hydroxyethyl)-5-(2,2,2-trifluoro-1-hydroxyethyl)-[1,1′-biphenyl]-3-yl)methoxy)phenyl)acetic acid (mixture of diastereomers);
2-(2-((3′-((S)-1-amino-2-hydroxyethyl)-5-(2,2,2-trifluoro-1-hydroxyethyl)-[1,1′-biphenyl]-3-yl)methoxy)phenyl)acetic acid (first diastereomer);
2-(2-((3′-((S)-1-amino-2-hydroxyethyl)-5-(2,2,2-trifluoro-1-hydroxyethyl)-[1,1′-biphenyl]-3-yl)methoxy)phenyl)acetic acid (second diastereomer);
2-(2-((3′-((R)-1-aminoethyl)-5-(2,2,2-trifluoro-1-hydroxyethyl)-[1,1′-biphenyl]-3-yl)methoxy)phenyl)acetic acid (mixture of diastereomers);
2-(2-((3′-((R)-1-aminoethyl)-5-(2,2,2-trifluoro-1-hydroxyethyl)-[1,1′-biphenyl]-3-yl)methoxy)phenyl)acetic acid (first diastereomers);
2-(2-((3′-((R)-1-aminoethyl)-5-(2,2,2-trifluoro-1-hydroxyethyl)-[1,1′-biphenyl]-3-yl)methoxy)phenyl)acetic acid (second diastereomer);
2-(2-((3′-((S)-1-amino-2-hydroxyethyl)-5-(cyclohexyl(hydroxy)methyl)-[1,1′-biphenyl]-3-yl)methoxy)phenyl)acetic acid (mixture of diastereomers);
2-(2-((3′-((S)-1-amino-2-hydroxyethyl)-5-(cyclohexyl(hydroxy)methyl)-[1,1′-biphenyl]-3-yl)methoxy)phenyl)acetic acid (first diastereomers);
2-(2-((3′-((S)-1-amino-2-hydroxyethyl)-5-(cyclohexyl(hydroxy)methyl)-[1,1′-biphenyl]-3-yl)methoxy)phenyl)acetic acid (second diastereomer);
(S)-2-(2-((3′-(1-amino-2-hydroxyethyl)-5-(2-hydroxypropan-2-yl)-[1,1′-biphenyl]-3-yl)methoxy)phenyl)acetic acid;
(S)-2-(2-((3′-(1-amino-2-hydroxyethyl)-6-methyl-[1,1′-biphenyl]-3-yl)methoxy)phenyl)acetic acid;
(S)-2-(2-((3′-(1-amino-2-hydroxyethyl)-2-methyl-[1,1′-biphenyl]-3-yl)methoxy)phenyl)acetic acid;
(R)-2-(2-((3′-(1-aminoethyl)-6-methyl-[1,1′-biphenyl]-3-yl)methoxy)phenyl)acetic acid;
(R)-2-(2-((3′-(1-aminoethyl)-2-methyl-[1,1′-biphenyl]-3-yl)methoxy)phenyl)acetic acid;
(R)-2-(2-((3′-(1-aminobutyl)-5-(hydroxymethyl)-[1,1′-biphenyl]-3-yl)methoxy)phenyl)acetic acid;
(R)-2-(2-((3′-(1-aminobutyl)-5-(methoxymethyl)-[1,1′-biphenyl]-3-yl)methoxy)phenyl)acetic acid;
2-(2-((3′-((R)-1-aminoethyl)-5-(1-methoxyethyl)-[1,1′-biphenyl]-3-yl)methoxy)phenyl)acetic acid;
(R)-2-(2-((3′-(1-aminoethyl)-2′-fluoro-5-(methoxymethyl)-[1,1′-biphenyl]-3-yl)methoxy)phenyl)acetic acid;
(R)-2-(2-((3′-(1-aminoethyl)-2′-fluoro-5-(hydroxymethyl)-[1,1′-biphenyl]-3-yl)methoxy)phenyl)acetic acid;
(R)-2-(2-((3′-(1-aminoethyl)-5-(methoxymethyl)-[1,1′-biphenyl]-3-yl)methoxy)phenyl)acetic acid;
2-(2-((3′-((R)-1-aminoethyl)-2′-fluoro-5-(1-methoxyethyl)-[1,1′-biphenyl]-3-yl)methoxy)phenyl)acetic acid;
2-(2-((3′-((S)-1-amino-2-hydroxyethyl)-5-(1-methoxyethyl)-[1,1′-biphenyl]-3-yl)methoxy)phenyl)acetic acid;
2-(2-((3′-((S)-1-amino-2-fluoroethyl)-5-(1-methoxyethyl)-[1,1′-biphenyl]-3-yl)methoxy)phenyl)acetic acid (mixture of diastereomers);
(S)-2-(2-((3′-(1-amino-2-hydroxyethyl)-6-fluoro-[1,1′-biphenyl]-3-yl)methoxy)phenyl)acetic acid;
(S)-2-(2-((3′-(1-amino-2-hydroxyethyl)-2-fluoro-[1,1′-biphenyl]-3-yl)methoxy)phenyl)acetic acid;
(R)-2-(2-((3′-(1-aminoethyl)-6-fluoro-[1,1′-biphenyl]-3-yl)methoxy)phenyl)acetic acid;
(R)-2-(2-((3′-(1-aminoethyl)-2-fluoro-[1,1′-biphenyl]-3-yl)methoxy)phenyl)acetic acid;
2-(2-((7-(3-(aminomethyl)phenyl)benzo[d][1,3]dioxol-5-yl)methoxy)phenyl)acetic acid;
(S)-2-(2-((3′-(1-amino-2-hydroxyethyl)-6-methoxy-[1,1′-biphenyl]-3-yl)methoxy)phenyl)acetic acid;
(S)-2-(2-((3′-(1-amino-2-hydroxyethyl)-5-((cyclopropylmethyl)amino)-[1,1′-biphenyl]-3-yl)methoxy)phenyl)acetic acid;
(S)-2-(2-((3′-(1-amino-2-hydroxyethyl)-5-(ethylamino)-[1,1′-biphenyl]-3-yl)methoxy)phenyl)acetic acid;
(S)-2-(2-((3′-(1-amino-2-hydroxyethyl)-5-(isopropylamino)-[1,1′-biphenyl]-3-yl)methoxy)phenyl)acetic acid;
(S)-2-(2-((3′-(1-amino-2-hydroxyethyl)-5-(methylamino)-[1,1′-biphenyl]-3-yl)methoxy)phenyl)acetic acid;
(S)-2-(2-((3′-(1-amino-2-hydroxyethyl)-5-(dimethylamino)-[1,1′-biphenyl]-3-yl)methoxy)phenyl)acetic acid;
(S)-2-(2-((5-amino-3′-(1-amino-2-hydroxyethyl)-[1,1′-biphenyl]-3-yl)methoxy)phenyl)acetic acid;
2-(2-((3′-(aminomethyl)-5-methoxy-[1,1′-biphenyl]-3-yl)methoxy)phenyl)acetic acid;
(R)-2-(2-((3′-(aminomethyl)-5-((tetrahydrofuran-2-yl)methoxy)-[1,1′-biphenyl]-3-yl)methoxy)phenyl)acetic acid;
2-(2-((3′-(aminomethyl)-5-(cyclopropylmethoxy)-[1,1′-biphenyl]-3-yl)methoxy)phenyl)acetic acid;
2-(2-((3′-(aminomethyl)-2′,6-difluoro-[1,1′-biphenyl]-3-yl)methoxy)phenyl)acetic acid;
(R)-2-(2-((3-(2-(aminomethyl)pyridin-4-yl)-5-((tetrahydrofuran-2-yl)methoxy)benzyl)oxy)phenyl)acetic acid;
(R)-2-(2-((3-(4-(aminomethyl)pyridin-2-yl)-5-((tetrahydrofuran-2-yl)methoxy)benzyl)oxy)phenyl)acetic acid;
(R)-2-(2-((3-(5-(aminomethyl)pyridin-3-yl)-5-((tetrahydrofuran-2-yl)methoxy)benzyl)oxy)phenyl)acetic acid;
2-(2-((3′-(aminomethyl)-4′-fluoro-[1,1′-biphenyl]-3-yl)methoxy)phenyl)acetic acid;
2-(2-((5′-(aminomethyl)-2′-fluoro-[1,1′-biphenyl]-3-yl)methoxy)phenyl)acetic acid;
2-(2-((3′-(1H-imidazol-2-y)-[1,1′-biphenyl]-3-yl)methoxy)phenyl)acetic acid;
2-(2-((3-(2-(aminomethyl)pyridin-4-yl)benzyl)oxy)phenyl)acetic acid;
2-(2-((3′-(aminomethyl)-2′-fluoro-[1,1′-biphenyl]-3-yl)methoxy)phenyl)acetic acid;
(R)-2-(2-((3-(6-(1-aminoethyl)pyridin-2-yl)benzyl)oxy)phenyl)acetic acid;
2-(2-((3′-(pyrrolidin-2-y)-[1,1′-biphenyl]-3-yl)methoxy)phenyl)acetic acid;
(S)-2-(2-((3-(6-(1-amino-2-hydroxyethyl)pyridin-2-yl)benzyl)oxy)phenyl)acetic acid;
(R)-2-(2-((3′-(1-aminoethyl)-2′-fluoro-[1,1′-biphenyl]-3-yl)methoxy)phenyl)acetic acid;
(R)-2-(2-((3-(6-(aminomethyl)pyridin-2-yl)-5-((tetrahydrofuran-2-yl)methoxy)benzyl)oxy)phenyl)acetic acid;
2-(2-((3-(6-(aminomethyl)pyridin-2-yl)benzyl)oxy)phenyl)acetic acid;
2-(2-((3′-(aminomethyl)-5-((cyclopropylmethyl)amino)-[1,1′-biphenyl]-3-yl)methoxy)phenyl)acetic acid;
(S)-2-(2-((3′-(aminomethyl)-5-(((tetrahydrofuran-2-yl)methyl)amino)-[1,1′-biphenyl]-3-yl)methoxy)phenyl)acetic acid;
(S)-2-(2-((3′-(1-amino-2-hydroxyethyl)-2′-chloro-[1,1′-biphenyl]-3-yl)methoxy)phenyl)acetic acid;
(R)-2-(2-((3′-(1-aminoethyl)-2′-chloro-[1,1′-biphenyl]-3-yl)methoxy)phenyl)acetic acid;
2-(2-((3′-(aminomethyl)-2′-chloro-[1,1′-biphenyl]-3-yl)methoxy)phenyl)acetic acid;
(S)-2-(2-((3′-(1-amino-2-hydroxyethyl)-2′-methyl-[1,1′-biphenyl]-3-yl)methoxy)phenyl)acetic acid;
(R)-2-(2-((3-(2-(1-aminoethyl)pyridin-4-yl)benzyl)oxy)phenyl)acetic acid;
(S)-2-(2-((3′-(1-amino-2-hydroxyethyl)-2′-fluoro-[1,1′-biphenyl]-3-yl)methoxy)phenyl)acetic acid;
(S)-2-(2-((3′-(2-amino-1-hydroxypropan-2-y)-[1,1′-biphenyl]-3-yl)methoxy)phenyl)acetic acid;
(S)-2-(2-((3-(2-(1-amino-2-hydroxyethyl)pyridin-4-yl)benzyl)oxy)phenyl)acetic acid;
(S)-2-(2-((3′-(1-aminoethyl)-2′-fluoro-[1,1′-biphenyl]-3-yl)methoxy)phenyl)acetic acid;
2-(2-((3-(2-(aminomethyl)-3-fluoropyridin-4-yl)benzyl)oxy)phenyl)acetic acid;
2-(2-((3-(2-(aminomethyl)pyrimidin-4-yl)benzyl)oxy)phenyl)acetic acid;
2-(2-((3′-(aminomethyl)-2′-methyl-[1,1′-biphenyl]-3-yl)methoxy)phenyl)acetic acid;
2-(2-((3′-(aminomethyl)-2′-hydroxy-[1,1′-biphenyl]-3-yl)methoxy)phenyl)acetic acid;
2-(2-((3′-(aminomethyl)-2′-methoxy-[1,1′-biphenyl]-3-yl)methoxy)phenyl)acetic acid;
(S)-2-(2-((3′-(1-amino-2-hydroxyethyl)-[1,1′-biphenyl]-3-yl)methoxy)phenyl)acetamide;
(S)-2-(2-((3′-(1-amino-2-hydroxyethyl)-5-(cyclopropylmethoxy)-[1,1′-biphenyl]-3-yl)methoxy)phenyl)acetic acid;
(R)-2-(2-((3′-(1-aminoethyl)-5-(cyclopropylmethoxy)-2′-fluoro-[1,1′-biphenyl]-3-yl)methoxy)phenyl)acetic acid;
2-(2-((3′-(aminomethyl)-2′-fluoro-6-methyl-[1,1′-biphenyl]-3-yl)methoxy)phenyl)acetic acid;
(R)-2-(2-((3′-(1-aminoethyl)-5-((cyclopropylmethyl)amino)-2′-fluoro-[1,1′-biphenyl]-3-yl)methoxy)phenyl)acetic acid;
(R)-2-(2-((3′-(1-aminoethyl)-5-(ethylamino)-2′-fluoro-[1,1′-biphenyl]-3-yl)methoxy)phenyl)acetic acid;
(R)-2-(2-((3′-(1-aminoethyl)-2′-fluoro-5-(isopropylamino)-[1,1′-biphenyl]-3-yl)methoxy)phenyl)acetic acid;
2-(2-((3′-((R)-1-aminoethyl)-2′-fluoro-5-((((R)-tetrahydrofuran-2-yl)methyl)amino)-[1,1′-biphenyl]-3-yl)methoxy)phenyl)acetic acid;
2-(2-((5-amino-3′-(aminomethyl)-4-fluoro-[1,1′-biphenyl]-3-yl)methoxy)phenyl)acetic acid;
(±)-2-(2-((3′-(aminomethyl)-4-fluoro-5-(((tetrahydrofuran-2-yl)methyl)amino)-[1,1′-biphenyl]-3-yl)methoxy)phenyl)acetic acid;
(S)-2-(2-((5-amino-3′-(1-amino-2-hydroxyethyl)-4-fluoro-[1,1′-biphenyl]-3-yl)methoxy)phenyl)acetic acid;
2-(2-((3′-((S)-1-amino-2-hydroxyethyl)-4-fluoro-5-(((tetrahydrofuran-2-yl)methyl)amino)-[1,1′-biphenyl]-3-yl)methoxy)phenyl)acetic acid (mixture of diastereomers);
(±)-2-(2-((3′-(aminomethyl)-6-fluoro-5-(((tetrahydrofuran-2-yl)methyl)amino)-[1,1′-biphenyl]-3-yl)methoxy)phenyl)acetic acid;
2-(2-((3′-((S)-1-amino-2-hydroxyethyl)-6-fluoro-5-(((tetrahydrofuran-2-yl)methyl)amino)-[1,1′-biphenyl]-3-yl)methoxy)phenyl)acetic acid (mixture of diastereomers);
2-(2-((3-(4-(aminomethyl)pyrimidin-2-yl)benzyl)oxy)phenyl)acetic acid;
2-(2-((3′-(aminomethyl)-[1,1′-biphenyl]-3-yl)methoxy)phenyl)acetic acid;
2-(2-((6-(3-(aminomethyl)phenyl)pyridin-2-yl)methoxy)phenyl)acetic acid;
(R)-2-(2-((3′-(1-aminoethyl)-[1,1′-biphenyl]-3-yl)methoxy)phenyl)acetic acid;
(±)-2-(2-((6-(3-(aminomethyl)phenyl)-2,3-dihydro-1H-inden-1-yl)oxy)phenyl)acetic acid;
(±)-2-(2-(1-(3′-(aminomethyl)-[1,1′-biphenyl]-3-yl)ethoxy)phenyl)acetic acid;
2-(2-((4-(3-(aminomethyl)phenyl)pyridin-2-yl)methoxy)phenyl)acetic acid;
2-(2-((3′-(aminomethyl)-4-(trifluoromethyl)-[1,1′-biphenyl]-3-yl)methoxy)phenyl)acetic acid;
2-(2-((3′-(aminomethyl)-4-fluoro-[1,1′-biphenyl]-3-yl)methoxy)phenyl)acetic acid;
(S)-2-(2-((3′-(1-amino-2-hydroxyethyl)-4-(trifluoromethyl)-[1,1′-biphenyl]-3-yl)methoxy)phenyl)acetic acid;
2-(2-((3′-(aminomethyl)-[1,1′-biphenyl]-3-yl)methoxy)-5-bromophenyl)acetic acid;
(S)-2-(2-((3′-(1-amino-2-methoxyethyl)-[1,1′-biphenyl]-3-yl)methoxy)phenyl)acetic acid;
(S)-2-(2-((4-(3-(1-amino-2-hydroxyethyl)phenyl)pyridin-2-yl)methoxy)phenyl)acetic acid;
(±)-2-(2-((3′-(2-hydroxy-1-(methylamino)ethyl)-[1,1′-biphenyl]-3-yl)methoxy)phenyl)acetic acid;
(±)-2-(2-((6-(3-((S)-1-amino-2-hydroxyethyl)phenyl)-2,3-dihydro-1H-inden-1-yl)oxy)phenyl)acetic acid
(S)-2-(2-((3′-(1-amino-2-ethoxyethyl)-[1,1′-biphenyl]-3-yl)methoxy)phenyl)acetic acid;
2-(2-((3′-(aminomethyl)-[1,1′-biphenyl]-3-yl)methoxy)-3-bromophenyl)acetic acid;
(+) or (−)-(2-((7-(3-(aminomethyl)phenyl)-1,2,3,4-tetrahydronaphthalen-1-yl)oxy)phenyl)acetic acid;
(−) or (+)-(2-((7-(3-(aminomethyl)phenyl)-1,2,3,4-tetrahydronaphthalen-1-yl)oxy)phenyl)acetic acid;
2-(2-((4-(5-(aminomethyl)-2-fluorophenyl)pyridin-2-yl)methoxy)phenyl)acetic acid;
(+) or (−)-2-(2-((6-(3-(aminomethyl)phenyl)chroman-4-yl)oxy)phenyl)acetic acid;
(−) or (+)-2-(2-((6-(3-(aminomethyl)phenyl)chroman-4-yl)oxy)phenyl)acetic acid;
2-(2-((6-(3-(aminomethyl)phenyl)-1-tosyl-1H-indazol-4-yl)methoxy)phenyl)acetic acid;
2-(2-((6-(3-(aminomethyl)phenyl)-1H-indazol-4-yl)methoxy)phenyl)acetic acid;
(R)-2-(2-((4-(3-(1-aminoethyl)phenyl)pyridin-2-yl)methoxy)phenyl)acetic acid;
2-(2-((7-(3-((S)-1-amino-2-hydroxyethyl)phenyl)-1,2,3,4-tetrahydronaphthalen-1-yl)oxy)phenyl)acetic acid (mixture of diastereomers);
2-(2-((4-(3-(aminomethyl)-2-fluorophenyl)pyridin-2-yl)methoxy)phenyl)acetic acid;
(±)-2-(2-(1-(3′-(aminomethyl)-2′-fluoro-[1,1′-biphenyl]-3-yl)ethoxy)phenyl)acetic acid;
(S)-2-(2-((4-(3-(1-amino-2-methoxyethyl)phenyl)pyridin-2-yl)methoxy)phenyl)acetic acid;
(S)-2-(2-((4-(3-(1-amino-2-hydroxyethyl)phenyl)-6-methylpyridin-2-yl)methoxy)phenyl)acetic acid;
(S)-2-(2-((6-(3-(1-amino-2-hydroxyethyl)phenyl)-1-tosyl-1H-indazol-4-yl)methoxy)phenyl)acetic acid;
2-(2-((3′-(aminomethyl)-5′-fluoro-[1,1′-biphenyl]-3-yl)methoxy)phenyl)acetic acid;
(S)-2-(2-((3′-(1-amino-2-hydroxyethyl)-4-(trifluoromethoxy)-[1,1′-biphenyl]-3-yl)methoxy)phenyl)acetic acid;
(±)-2-(2-((3′-(1,2-diamino-2-oxoethyl)-[1,1′-biphenyl]-3-yl)methoxy)phenyl)acetic acid;
(S)-2-(2-((3′-(1-amino-2-hydroxyethyl)-5′-fluoro-[1,1′-biphenyl]-3-yl)methoxy)phenyl)acetic acid;
(−)-2-(2-((3′-(1-amino-2-fluoroethyl)-[1,1′-biphenyl]-3-yl)methoxy)phenyl)acetic acid;
(+)-2-(2-((3′-(1-amino-2-fluoroethyl)-[1,1′-biphenyl]-3-yl)methoxy)phenyl)acetic acid;
(R)-2-(2-((3′-(1-aminopropyl)-[1,1′-biphenyl]-3-yl)methoxy)phenyl)acetic acid;
2-(2-((3′-((1S,2S)-1-amino-2-hydroxypropyl)-[1,1′-biphenyl]-3-yl)methoxy)phenyl)acetic acid;
(R)-2-(2-((3′-(1-aminobutyl)-[1,1′-biphenyl]-3-yl)methoxy)phenyl)acetic acid;
(R)-2-(2-((3′-(1-amino-2-methylpropyl)-[1,1′-biphenyl]-3-yl)methoxy)phenyl)acetic acid;
2-(2-((3′-((1S,2R)-1-amino-2-hydroxypropyl)-[1,1′-biphenyl]-3-yl)methoxy)phenyl)acetic acid;
(R)-2-(2-((3-(6-(1-amino-3-hydroxypropyl)pyridin-2-yl)benzyl)oxy)phenyl)acetic acid;
(R)-2-(2-((3′-(1-amino-3-hydroxypropyl)-[1,1′-biphenyl]-3-yl)methoxy)phenyl)acetic acid;
(R)-2-(2-((3′-(1-amino-3-hydroxypropyl)-2′-fluoro-[1,1′-biphenyl]-3-yl)methoxy)phenyl)acetic acid;
2-(2-((3-(2-(aminomethyl)thiazol-4-yl)benzyl)oxy)phenyl)acetic acid;
2-(2-((3′-(1H-tetrazol-5-y)-[1,1′-biphenyl]-3-yl)methoxy)phenyl)acetic acid;
2-(2-((3′-carbamimidoyl-[1,1′-biphenyl]-3-yl)methoxy)phenyl)acetic acid;
(S)-2-amino-2-(3′-((2-(carboxymethyl)phenoxy)methyl)-[1,1′-biphenyl]-3-yl)acetic acid;
(R)-2-(2-((3′-(1-aminobutyl)-[1,1′-biphenyl]-3-yl)methoxy)-3-fluorophenyl)acetic acid;
(R)-2-(2-((3′-(1-aminobutyl)-2′-fluoro-[1,1′-biphenyl]-3-yl)methoxy)phenyl)acetic acid;
(R)-2-(2-((3-(6-(1-aminobutyl)pyridin-2-yl)benzyl)oxy)phenyl)acetic acid;
(R)-2-(2-((3-(2-(1-aminobutyl)pyridin-4-yl)benzyl)oxy)phenyl)acetic acid;
2-(2-((3′-(amino(phenyl)methyl)-[1,1′-biphenyl]-3-yl)methoxy)phenyl)acetic acid;
(R)-2-(2-((3′-(1-aminopentyl)-[1,1′-biphenyl]-3-yl)methoxy)phenyl)acetic acid;
(R)-2-(2-((3′-(1-amino-2-cyanoethyl)-[1,1′-biphenyl]-3-yl)methoxy)phenyl)acetic acid;
(±)-2-(2-((3′-(1-amino-2-fluoroethyl)-2′-fluoro-[1,1′-biphenyl]-3-yl)methoxy)phenyl)acetic acid;
(R)-2-(2-((3′-(amino(cyclopropyl)methyl)-[1,1′-biphenyl]-3-yl)methoxy)phenyl)acetic acid;
(R)-2-(2-((3′-(1-amino-3,3,3-trifluoropropyl)-[1,1′-biphenyl]-3-yl)methoxy)phenyl)acetic acid;
(R)-2-(2-((3′-(1-amino-2-cyclopropylethyl)-[1,1′-biphenyl]-3-yl)methoxy)phenyl)acetic acid;
(R)-2-(2-((3′-(1-aminobutyl)-5-((cyclopropylmethyl)amino)-[1,1′-biphenyl]-3-yl)methoxy)phenyl)acetic acid;
(R)-2-(2-((3′-(1-aminobutyl)-2′-fluoro-5-morpholino-[1,1′-biphenyl]-3-yl)methoxy)phenyl)acetic acid;
(R)-2-(2-((3′-(1-aminobutyl)-2′-fluoro-5-(pyrrolidin-1-yl)-[1,1′-biphenyl]-3-yl)methoxy)phenyl)acetic acid;
(R)-2-(2-((3′-(1-aminobutyl)-5-((cyclopropylmethyl)amino)-2′-fluoro-[1,1′-biphenyl]-3-yl)methoxy)phenyl)acetic acid;
2-(2-((3′-((R)-1-aminobutyl)-2′-fluoro-5-((((S)-tetrahydrofuran-2-yl)methyl)amino)-[1,1′-biphenyl]-3-yl)methoxy)phenyl)acetic acid;
(R)-2-(2-((3-(1-aminobutyl)-2-fluoro-[1,1′:3′,1″-terphenyl]-5′-yl)methoxy)phenyl)acetic acid;
(R)-2-(2-((3′-(1-aminobutyl)-5-((2-methoxyethyl)amino)-[1,1′-biphenyl]-3-yl)methoxy)phenyl)acetic acid;
(R)-2-(2-((3′-(1-aminobutyl)-5-cyclopropyl-2′-fluoro-[1,1′-biphenyl]-3-yl)methoxy)phenyl)acetic acid;
(R)-2-(2-((3′-(1-aminobutyl)-2′-fluoro-5-(1-methyl-1H-pyrazol-4-yl)-[1,1′-biphenyl]-3-yl)methoxy)phenyl)acetic acid;
2-(2-((3′-(1-amino-2-fluoroethyl)-2′-fluoro-5-((((S)-tetrahydrofuran-2-yl)methyl)amino)-[1,1′-biphenyl]-3-yl)methoxy)phenyl)acetic acid (mixture of diastereomers);
tert-butyl 2-(2-((3′-(1-amino-2-fluoroethyl)-2′-fluoro-5-(methoxymethyl)-[1,1′-biphenyl]-3-yl)methoxy)phenyl)acetate;
(±)-2-(2-((3′-(1-amino-2-fluoroethyl)-2′-fluoro-5-(methoxymethyl)-[1,1′-biphenyl]-3-yl)methoxy)phenyl)acetic acid;
(±)-2-(2-((3′-(1-amino-2-fluoroethyl)-5-(cyclopropylmethoxy)-2′-fluoro-[1,1′-biphenyl]-3-yl)methoxy)phenyl)acetic acid;
(±)-2-(2-((5-(1-amino-2,2,2-trifluoroethyl)-3′-(aminomethyl)-[1,1′-biphenyl]-3-yl)methoxy)phenyl)acetic acid;
(±)-2-(2-((3′-(aminomethyl)-5-(2,2,2-trifluoro-1-(methylamino)ethyl)-[1,1′-biphenyl]-3-yl)methoxy)phenyl)acetic acid;
(±)-2-(2-((3′-(aminomethyl)-5-(1-(dimethylamino)-2,2,2-trifluoroethyl)-[1,1′-biphenyl]-3-yl)methoxy)phenyl)acetic acid;
(±)-2-(2-((3′-(aminomethyl)-5-(2,2,2-trifluoro-1-(phenylamino)ethyl)-[1,1′-biphenyl]-3-yl)methoxy)phenyl)acetic acid;
(±)-2-(2-((3′-(aminomethyl)-5-(1-benzamido-2,2,2-trifluoroethyl)-[1,1′-biphenyl]-3-yl)methoxy)phenyl)acetic acid;
2-(2-((3′-(aminomethyl)-5-(((2,2,2-trifluoroethyl)amino)methyl)-[1,1′-biphenyl]-3-yl)methoxy)phenyl)acetic acid;
(±)-2-(2-((3′-(aminomethyl)-5-(((1,1,1-trifluoropropan-2-yl)amino)methyl)-[1,1′-biphenyl]-3-yl)methoxy)phenyl)acetic acid;
2-(2-((3′-(aminomethyl)-5-((methyl(2,2,2-trifluoroethyl)amino)methyl)-[1,1′-biphenyl]-3-yl)methoxy)phenyl)acetic acid;
2-(2-((3′-(aminomethyl)-5-(1-methyl-1H-pyrazol-4-yl)-[1,1′-biphenyl]-3-yl)methoxy)phenyl)acetic acid;
2-(2-((3′-(aminomethyl)-5-(1H-indol-2-yl)-[1,1′-biphenyl]-3-yl)methoxy)phenyl)acetic acid; and
2-(2-((3′-(aminomethyl)-5-(1H-pyrazol-5-yl)-[1,1′-biphenyl]-3-yl)methoxy)phenyl)acetic acid.

Certain particularly preferred compounds of the twenty fifth embodiment include compounds selected from the group consisting of (R)-2-(2-((3′-(1-Aminoethyl)-5-(hydroxymethyl)-[1,1′-biphenyl]-3-yl)methoxy)phenyl)acetic acid;
(S)-2-(2-((3′-(1-Amino-2-hydroxyethyl)-[1,1′-biphenyl]-3-yl)methoxy)phenyl)acetic acid;
(S)-2-(2-((3′-(1-Amino-2-hydroxyethyl)-5-ethoxy-[1,1′-biphenyl]-3-yl)methoxy)phenyl)acetic acid;
(S)-2-(2-((3′-(1-Amino-2-hydroxyethyl)-5-(2-hydroxypropan-2-yl)-[1,1′-biphenyl]-3-yl)methoxy)phenyl)acetic acid;
(R)-2-(2-((3′-(1-Aminoethyl)-2′-fluoro-5-(methoxymethyl)-[1,1′-biphenyl]-3-yl)methoxy)phenyl)acetic acid;
(R)-2-(2-((3′-(1-Aminoethyl)-5-(methoxymethyl)-[1,1′-biphenyl]-3-yl)methoxy)phenyl)acetic acid;
(S)-2-(2-((3′-(1-Amino-2-hydroxyethyl)-5-((cyclopropylmethyl)amino)-[1,1′-biphenyl]-3-yl)methoxy)phenyl)acetic;
(S)-2-(2-((3′-(Aminomethyl)-5-(((tetrahydrofuran-2-yl)methyl)amino)-[1,1′-biphenyl]-3-yl)methoxy)phenyl)acetic acid;
(R)-2-(2-((3′-(1-aminoethyl)-5-(ethylamino)-2′-fluoro-[1,1′-biphenyl]-3-yl)methoxy)phenyl)acetic acid; and
2-(2-((6-(3-(Aminomethyl)phenyl)-1H-indazol-4-yl)methoxy)phenyl)acetic acid or a pharmaceutically acceptable salt thereof

In a twenty sixth embodiment, compounds of embodiment one are provided, which compounds, and salts thereof, are selected from the group consisting of
2-(2-((3′-((R)-1-aminoethyl)-2′-fluoro-5-(1-hydroxyethyl)-[1,1′-biphenyl]-3-yl)methoxy)phenyl)acetic acid (mixture of diastereomers);
(R)-2-(2-((3′-(1-aminoethyl)-2′-fluoro-5-(2-hydroxypropan-2-yl)-[1,1′-biphenyl]-3-yl)methoxy)phenyl)acetic acid;
(R)-2-(2-((3′-(1-aminoethyl)-5-(2-hydroxypropan-2-yl)-[1,1′-biphenyl]-3-yl)methoxy)phenyl)acetic acid;
(S)-2-(2-((3′-(1-aminoethyl)-2′-fluoro-5-(methoxymethyl)-[1,1′-biphenyl]-3-yl)methoxy)phenyl)acetic acid;
2-(2-((3′-((S)-1-aminoethyl)-2′-fluoro-5-(1-hydroxyethyl)-[1,1′-biphenyl]-3-yl)methoxy)phenyl)acetic acid;
(S)-2-(2-((3′-(1-aminoethyl)-5-(methoxymethyl)-[1,1′-biphenyl]-3-yl)methoxy)phenyl)acetic acid;
2-(2-((3′-((S)-1-aminoethyl)-5-(1-hydroxyethyl)-[1,1′-biphenyl]-3-yl)methoxy)phenyl)acetic acid;
(R)-2-(2-((3′-(1-aminoethyl)-2′-fluoro-5-((2,2,2-trifluoroethoxy)methyl)-[1,1′-biphenyl]-3-yl)methoxy)phenyl)acetic acid;
(R)-2-(2-((3′-(1-aminoethyl)-5-((2,2,2-trifluoroethoxy)methyl)-[1,1′-biphenyl]-3-yl)methoxy)phenyl)acetic acid;
(R)-2-(2-((3′-(1-aminoethyl)-2′-fluoro-5-(isopropoxymethyl)-[1,1′-biphenyl]-3-yl)methoxy)phenyl)acetic acid;
(R)-2-(2-((3′-(1-aminoethyl)-5-(isopropoxymethyl)-[1,1′-biphenyl]-3-yl)methoxy)phenyl)acetic acid;
(R)-2-(2-((3′-(1-aminoethyl)-5-(ethoxymethyl)-2′-fluoro-[1,1′-biphenyl]-3-yl)methoxy)phenyl)acetic acid;
(R)-2-(2-((3′-(1-aminoethyl)-5-(ethoxymethyl)-[1,1′-biphenyl]-3-yl)methoxy)phenyl)acetic acid;
2-(2-((3′-((R)-1-aminoethyl)-5-(((6-chlorochroman-4-yl)oxy)methyl)-2′-fluoro-[1,1′-biphenyl]-3-yl)methoxy)phenyl)acetic acid (mixture of diastereomers);
2-(2-((3′-((R)-1-aminoethyl)-5-(((6-chlorochroman-4-y)oxy)methyl)-[1,1′-biphenyl]-3-yl)methoxy)phenyl)acetic acid (mixture of diastereomers);
(R)-2-(2-((3′-(1-aminoethyl)-5-bromo-2′-fluoro-[1,1′-biphenyl]-3-yl)methoxy)phenyl)acetic acid;
(R)-2-(2-((3′-(1-aminoethyl)-2′-fluoro-5-(2-methoxyethyl)-[1,1′-biphenyl]-3-yl)methoxy)phenyl)acetic acid;
(R)-2-(2-((3′-(1-aminoethyl)-5-(2-methoxyethyl)-[1,1′-biphenyl]-3-yl)methoxy)phenyl)acetic acid;
(R)-2-(2-((3′-(1-aminoethyl)-5-((difluoromethoxy)methyl)-2′-fluoro-[1,1′-biphenyl]-3-yl)methoxy)phenyl)acetic acid;
2-(2-((3′-(2-Aminopropan-2-y)-2′-fluoro-[1,1′-biphenyl]-3-yl)methoxy)phenyl)acetic acid;
2-(2-((3′-(aminomethyl)-5-(1-isopropyl-1H-pyrazol-4-y)-[1,1′-biphenyl]-3-yl)methoxy)phenyl)acetic acid;
2-(2-((3′-(aminomethyl)-5-(1-isobutyl-1H-pyrazol-4-y)-[1,1′-biphenyl]-3-yl)methoxy)phenyl)acetic acid;
2-(2-((3′-(aminomethyl)-5-(1-(2-methoxyethyl)-1H-pyrazol-4-y)-[1,1′-biphenyl]-3-yl)methoxy)phenyl)acetic acid;
(R)-2-(2-((3′-(1-aminoethyl)-2′-fluoro-5-methyl-[1,1′-biphenyl]-3-yl)methoxy)phenyl)acetic acid;
(R)-2-(2-((6-(3-(1-aminoethyl)phenyl)-1H-indazol-4-yl)methoxy)phenyl)acetic acid;
(+)-2-(2-((3′-(1-amino-2-fluoroethyl)-2′-fluoro-5-(((R)-tetrahydrofuran-2-yl)methoxy)-[1,1′-biphenyl]-3-yl)methoxy)phenyl)acetic acid;
(R)-2-(2-((3′-(1-Aminoethyl)-2′-fluoro-5-(pyridin-2-ylmethoxy)-[1,1′-biphenyl]-3-yl)methoxy)phenyl)acetic acid;
(R)-2-(2-((3′-(1-aminoethyl)-2′-fluoro-5-(2-morpholinoethoxy)-[1,1′-biphenyl]-3-yl)methoxy)phenyl)acetic acid;
(R)-2-(2-((3′-(1-aminoethyl)-2′-fluoro-5-(thiazol-2-ylmethoxy)-[1,1′-biphenyl]-3-yl)methoxy)phenyl)acetic acid;
(R)-2-(2-((3′-(1-aminoethyl)-2′-fluoro-5-(2-fluoroethoxy)-[1,1′-biphenyl]-3-yl)methoxy)phenyl)acetic acid;
(R)-2-(2-((3′-(1-aminoethyl)-2′-fluoro-5-((tetrahydro-2H-pyran-4-yl)methoxy)-[1,1′-biphenyl]-3-yl)methoxy)phenyl)acetic acid;
(R)-2-(2-((3′-(1-aminoethyl)-2′-fluoro-5-((1-methyl-1H-pyrazol-3-yl)methoxy)-[1,1′-biphenyl]-3-yl)methoxy)phenyl)acetic acid;
(R)-2-(2-((3′-(1-aminoethyl)-2′-fluoro-5-(pyridin-4-ylmethoxy)-[1,1′-biphenyl]-3-yl)methoxy)phenyl)acetic acid;
(R)-2-(2-((3′-(1-aminoethyl)-2′-fluoro-5-(2-(pyrrolidin-1-yl)ethoxy)-[1,1′-biphenyl]-3-yl)methoxy)phenyl)acetic;
(R)-2-(2-((3′-(1-aminoethyl)-5-(cyclopentylmethoxy)-2′-fluoro-[1,1′-biphenyl]-3-yl)methoxy)phenyl)acetic acid;
R)-2-(2-((3′-(1-aminoethyl)-2′-fluoro-5-methoxy-[1,1′-biphenyl]-3-yl)methoxy)phenyl)acetic acid;
(R)-2-(2-((3′-(1-aminoethyl)-2′-fluoro-5-(pyrimidin-4-ylmethoxy)-[1,1′-biphenyl]-3-yl)methoxy)phenyl)acetic acid;
(R)-2-(2-((3′-(1-aminoethyl)-2′-fluoro-5-((1-methyl-1H-1,2,4-triazol-3-yl)methoxy)-[1,1′-biphenyl]-3-yl)methoxy)phenyl)acetic acid;
(R)-2-(2-((3′-(1-aminoethyl)-2′-fluoro-5-(pyridin-3-ylmethoxy)-[1,1′-biphenyl]-3-yl)methoxy)phenyl)acetic acid;
(S)-2-(2-((3′-(1-aminoethyl)-2′-fluoro-5-(pyridin-2-ylmethoxy)-[1,1′-biphenyl]-3-yl)methoxy)phenyl)acetic acid;
(S)-2-(2-((3′-(1-aminoethyl)-2′-fluoro-5-(thiazol-2-ylmethoxy)-[1,1′-biphenyl]-3-yl)methoxy)phenyl)acetic acid;
(S)-2-(2-((3′-(1-aminoethyl)-2′-fluoro-5-(2-fluoroethoxy)-[1,1′-biphenyl]-3-yl)methoxy)phenyl)acetic acid;
(S)-2-(2-((3′-(1-aminoethyl)-2′-fluoro-5-((1-methyl-1H-imidazol-5-yl)methoxy)-[1,1′-biphenyl]-3-yl)methoxy)phenyl)acetic acid;
(R)-2-(2-((3′-(1-aminoethyl)-2′-fluoro-5-((1-methyl-1H-imidazol-5-yl)methoxy)-[1,1′-biphenyl]-3-yl)methoxy)phenyl)acetic acid;
(R)-2-(2-((3′-(1-aminoethyl)-2′-fluoro-5-hydroxy-[1,1′-biphenyl]-3-yl)methoxy)phenyl)acetic acid;
2-(2-((3′-(1-amino-2-fluoroethyl)-2′-fluoro-[1,1′-biphenyl]-3-yl)methoxy)phenyl)acetic acid;
(S)-2-(2-((3′-(1-amino-2-fluoroethyl)-5-((cyclopropylmethyl)amino)-2′-fluoro-[1,1′-biphenyl]-3-yl)methoxy)phenyl)acetic acid;
(R)-2-(2-((3′-(1-amino-2-fluoroethyl)-5-((cyclopropylmethyl)amino)-2′-fluoro-[1,1′-biphenyl]-3-yl)methoxy)phenyl)acetic acid;
(S)-2-(2-((3′-(1-aminoethyl)-5-((cyclopentylmethyl)amino)-2′-fluoro-[1,1′-biphenyl]-3-yl)methoxy)phenyl)acetic acid;
(R)-2-(2-((3′-(1-aminoethyl)-5-((cyclopentylmethyl)amino)-2′-fluoro-[1,1′-biphenyl]-3-yl)methoxy)phenyl)acetic acid;
2-(2-((3′-(1-amino-2-fluoroethyl)-5-((cyclopentylmethyl)amino)-2′-fluoro-[1,1′-biphenyl]-3-yl)methoxy)phenyl)acetic acid;
(R)-2-(2-((3′-(1-aminoethyl)-5-((cyclopentylmethyl)(methyl)amino)-2′-fluoro-[1,1′-biphenyl]-3-yl)methoxy)phenyl)acetic acid;
2-(2-((3′-((R)-1-aminoethyl)-2′-fluoro-5-(methyl((tetrahydrofuran-2-yl)methyl)amino)-[1,1′-biphenyl]-3-yl)methoxy)phenyl)acetic acid;
2-(2-((3′-((R)-1-aminoethyl)-2′-fluoro-5-(methyl(((S)-tetrahydrofuran-2-yl)methyl)amino)-[1,1′-biphenyl]-3-yl)methoxy)phenyl)acetic acid;
2-(2-((3′-((R)-1-aminoethyl)-2′-fluoro-5-(methyl(((R)-tetrahydrofuran-2-yl)methyl)amino)-[1,1′-biphenyl]-3-yl)methoxy)phenyl)acetic acid;
2-(2-((3′-((R)-1-aminoethyl)-5-(ethyl(((S)-tetrahydrofuran-2-yl)methyl)amino)-2′-fluoro-[1,1′-biphenyl]-3-yl)methoxy)phenyl)acetic acid;
(R)-2-(2-((3′-(1-aminoethyl)-2′-fluoro-5-((2-fluoroethyl)amino)-[1,1′-biphenyl]-3-yl)methoxy)phenyl)acetic acid;
(R)-2-(2-((3′-(1-aminoethyl)-2′-fluoro-5-((2-fluoroethyl)(methyl)amino)-[1,1′-biphenyl]-3-yl)methoxy)phenyl)acetic acid;
(R)-2-(2-((3′-(1-aminoethyl)-5-((2,2-difluoroethyl)amino)-2′-fluoro-[1,1′-biphenyl]-3-yl)methoxy)phenyl)acetic acid;
(R)-2-(2-((3′-(1-aminoethyl)-5-((2,2-difluoroethyl)(methyl)amino)-2′-fluoro-[1,1′-biphenyl]-3-yl)methoxy)phenyl)acetic acid;
(S)-2-(2-((3′-(1-aminoethyl)-2′-fluoro-5-(2-(pyridin-4-y)ethyl)-[1,1′-biphenyl]-3-yl)methoxy)phenyl)acetic acid;
(R)-2-(2-((3′-(1-aminoethyl)-2′-fluoro-5-(2-(pyridin-4-y)ethyl)-[1,1′-biphenyl]-3-yl)methoxy)phenyl)acetic acid;
2-(2-((3′-(1-amino-2-fluoroethyl)-2′-fluoro-5-(2-(pyridin-4-y)ethyl)-[1,1′-biphenyl]-3-yl)methoxy)phenyl)acetic acid;
(R)-2-(2-((3′-(1-aminoethyl)-5-(dimethyl carbamoyl)-2′-fluoro-[1,1′-biphenyl]-3-yl)methoxy)phenyl)acetic acid;
(R)-2-(2-((3′-(1-aminoethyl)-5-chloro-2′-fluoro-[1,1′-biphenyl]-3-yl)methoxy)phenyl)acetic acid;
(R)-2-(2-((3′-(1-aminoethyl)-2′-fluoro-5-((pyridin-2-ylmethyl)amino)-[1,1′-biphenyl]-3-yl)methoxy)phenyl)acetic acid;
(R)-2-(2-((3′-(1-aminoethyl)-5-(cyclopentylamino)-2′-fluoro-[1,1′-biphenyl]-3-yl)methoxy)phenyl)acetic acid;
(R)-2-(2-((3′-(1-aminoethyl)-2′-fluoro-5-(pyrrolidin-1-yl)-[1,1′-biphenyl]-3-yl)methoxy)phenyl)acetic acid;
2-(2-((6-(3-((R)-1-aminoethyl)-2-fluorophenyl)-8-((((R)-tetrahydrofuran-2-yl)methyl)amino)chroman-4-yl)oxy)phenyl)acetic acid;
2-(2-((6-(3-((R)-1-aminoethyl)-2-fluorophenyl)-8-((cyclopropylmethyl)amino)chroman-4-yl)oxy)phenyl)acetic acid;
2-(2-((7-(3-((R)-1-aminoethyl)-2-fluorophenyl)-5-((((R)-tetrahydrofuran-2-yl)methyl)amino)-1,2,3,4-tetrahydronaphthalen-1-yl)oxy)phenyl)acetic acid;
2-(2-((7-(3-((R)-1-aminoethyl)-2-fluorophenyl)-5-((cyclopropylmethyl)amino)-1,2,3,4-tetrahydronaphthalen-1-yl)oxy)phenyl)acetic acid;
(R)-2-(2-(((3′-(1-aminoethyl)-5-((cyclopropylmethyl)amino)-2′-fluoro-[1,1′-biphenyl]-3-yl)(methyl)amino)methyl)phenyl)acetic acid;
2-(2-(((3′-((R)-1-aminoethyl)-2′-fluoro-5-((((S)-tetrahydrofuran-2-yl)methyl)amino)-[1,1′-biphenyl]-3-yl)(methyl)amino)methyl)phenyl)acetic acid;
(R)-2-(2-(((3′-(1-aminoethyl)-5-((2,2-difluoroethyl)amino)-2′-fluoro-[1,1′-biphenyl]-3-yl)(methyl)amino)methyl)phenyl)acetic acid;
(R)-2-(2-((3′-(1-aminoethyl)-5-((cyclopropylamino)methyl)-2′-fluoro-[1,1′-biphenyl]-3-yl)methoxy)phenyl)acetic acid;
(R)-2-(2-((3′-(1-aminoethyl)-2′-fluoro-5-(morpholinomethyl)-[1,1′-biphenyl]-3-yl)methoxy)phenyl)acetic acid;
(R)-2-(2-((3′-(1-aminoethyl)-2′-fluoro-5-(((1-methyl-1H-pyrazol-3-yl)amino)methyl)-[1,1′-biphenyl]-3-yl)methoxy)phenyl)acetic acid;
2-(2-((3′-((R)-1-aminoethyl)-2′-fluoro-5-(((tetrahydrofuran-3-yl)amino)methyl)-[1,1′-biphenyl]-3-yl)methoxy)phenyl)acetic acid;
2-(2-((3′-((R)-1-aminoethyl)-5-(((2S,6R)-2,6-dimethylmorpholino)methyl)-2′-fluoro-[1,1′-biphenyl]-3-yl)methoxy)phenyl)acetic acid;
(R)-2-(2-((5-((1,4-oxazepan-4-yl)methyl)-3′-(1-aminoethyl)-2′-fluoro-[1,1′-biphenyl]-3-yl)methoxy)phenyl)acetic acid;
(R)-2-(2-((3′-(1-aminoethyl)-2′-fluoro-5-(pyrrolidin-1-ylmethyl)-[1,1′-biphenyl]-3-yl)methoxy)phenyl)acetic acid;
(+) or (−)-2-(2-((3′-(1-Amino-2-fluoroethyl)-2′-fluoro-5-(morpholinomethyl)-[1,1′-biphenyl]-3-yl)methoxy)phenyl)acetic acid;
(R)-2-(2-((3′-(1-Aminoethyl)-2′-fluoro-5-((1-methyl-1H-pyrazol-3-yl)ethynyl)-[1,1′-biphenyl]-3-yl)methoxy)phenyl)acetic acid;
(R)-2-(2-((3′-(1-aminoethyl)-2′-fluoro-5-(2-(1-methyl-1H-pyrazol-3-yl)ethyl)-[1,1′-biphenyl]-3-yl)methoxy)phenyl)acetic acid;
(R)-2-(2-((7-(3-(1-Aminoethyl)-2-fluorophenyl)-3,4-dihydroquinolin-1 (2H)-yl)methyl)phenyl)acetic acid;
(R)-2-(2-(((3′-(1-aminoethyl)-2′-fluoro-[1,1′-biphenyl]-3-yl)(methyl)amino)methyl)phenyl)acetic acid;
(R)-2-(2-((5-amino-3′-(1-aminoethyl)-2′,4-difluoro-[1,1′-biphenyl]-3-yl)methoxy)phenyl)acetic acid;
2-(2-((3′-((R)-1-aminoethyl)-2′,4-difluoro-5-(((tetrahydrofuran-2-yl)methyl)amino)-[1,1′-biphenyl]-3-yl)methoxy)phenyl)acetic acid (mixture of diastereomers);
2-(2-((3′-((R)-1-aminoethyl)-2′,6-difluoro-5-(((tetrahydrofuran-2-yl)methyl)amino)-[1,1′-biphenyl]-3-yl)methoxy)phenyl)acetic acid;
(R)-2-(2-((3′-(1-aminoethyl)-2′-fluoro-5-((2-methoxyethyl)amino)-[1,1′-biphenyl]-3-yl)methoxy)phenyl)acetic acid;
(R)-2-(2-((3′-(1-aminoethyl)-2′-fluoro-5-(isobutylamino)-[1,1′-biphenyl]-3-yl)methoxy)phenyl)acetic acid;
2-(2-((3-(6-((R)-1-aminoethyl)pyridin-2-yl)-5-((((R)-tetrahydrofuran-2-yl)methyl)amino)benzyl)oxy)phenyl)acetic acid;
(R)-2-(2-((3′-(1-aminoethyl)-2′-fluoro-5-((2,2,2-trifluoroethyl)amino)-[1,1′-biphenyl]-3-yl)methoxy)phenyl)acetic acid;
(R)-2-(2-((3′-(1-aminoethyl)-2′-fluoro-5-(((1-methylcyclopropyl)methyl)amino)-[1,1′-biphenyl]-3-yl)methoxy)phenyl)acetic acid;
(R)-2-(2-((5-acetamido-3′-(1-aminoethyl)-2′-fluoro-[1,1′-biphenyl]-3-yl)methoxy)phenyl)acetic acid;
(R)-2-(2-((3′-(1-aminoethyl)-2′-fluoro-5-(N-methyl acetamido)-[1,1′-biphenyl]-3-yl)methoxy)phenyl)acetic acid;
(R)-2-(2-((3′-(1-aminoethyl)-2′-fluoro-5-(1-methyl-1H-pyrazol-4-yl)-[1,1′-biphenyl]-3-yl)methoxy)phenyl)acetic acid;
(R)-2-(2-((3′-(1-aminoethyl)-2′-fluoro-5-(2,2,2-trifluoroethoxy)-[1,1′-biphenyl]-3-yl)methoxy)phenyl)acetic acid;
2-(2-((3′-((S)-1-amino-2-hydroxyethyl)-2′-fluoro-5-(((R)-tetrahydrofuran-2-yl)methoxy)-[1,1′-biphenyl]-3-yl)methoxy)phenyl)acetic acid;
2-(2-((3′-((R)-1-aminoethyl)-2′-fluoro-5-(((R)-tetrahydrofuran-2-yl)methoxy)-[1,1′-biphenyl]-3-yl)methoxy)phenyl)acetic acid;
2-(2-((3′-((S)-1-aminoethyl)-2′-fluoro-5-(((R)-tetrahydrofuran-2-yl)methoxy)-[1,1′-biphenyl]-3-yl)methoxy)phenyl)acetic acid;
(R)-2-(2-((3′-(aminomethyl)-2′-fluoro-5-((tetrahydrofuran-2-yl)methoxy)-[1,1′-biphenyl]-3-yl)methoxy)phenyl)acetic acid;
2-(2-((3-(6-((R)-1-aminoethyl)pyridin-2-yl)-5-(((R)-tetrahydrofuran-2-yl)methoxy)benzyl)oxy)phenyl)acetic acid;
2-(2-((3′-((R)-1-aminoethyl)-2′-fluoro-5-(2-(tetrahydrofuran-2-yl)ethyl)-[1,1′-biphenyl]-3-yl)methoxy)phenyl)acetic acid;
(R)-2-(2-((3′-(1-aminoethyl)-5-(2-cyclopentylethyl)-2′-fluoro-[1,1′-biphenyl]-3-yl)methoxy)phenyl)acetic acid;
2-(2-((3′-((R)-1-aminoethyl)-2′-fluoro-5-(2-(pyrrolidin-2-y)ethyl)-[1,1′-biphenyl]-3-yl)methoxy)phenyl)acetic acid;
2-(2-((3′-((R)-1-aminoethyl)-2′-fluoro-5-(2-(morpholin-2-y)ethyl)-[1,1′-biphenyl]-3-yl)methoxy)phenyl)acetic acid;
2-(2-((3′-((R)-1-aminoethyl)-2′-fluoro-5-(2-(1-methyl pyrrolidin-2-y)ethyl)-[1,1′-biphenyl]-3-yl)methoxy)phenyl)acetic acid;
(R)-2-(2-((3′-(1-Aminoethyl)-2′-fluoro-5-(piperidin-1-yl)-[1,1′-biphenyl]-3-yl)methoxy)phenyl)acetic acid;
(R)-2-(2-((3′-(1-aminoethyl)-5-((cyclopropylmethyl) (ethyl)amino)-2′-fluoro-[1,1′-biphenyl]-3-yl)methoxy)phenyl)acetic acid;
(R)-2-(2-((3′-(1-aminoethyl)-5-cyclohexyl-2′-fluoro-[1,1′-biphenyl]-3-yl)methoxy)phenyl)acetic acid;
(R)-2-(2-((3′-(1-aminoethyl)-5-cyclopentyl-2′-fluoro-[1,1′-biphenyl]-3-yl)methoxy)phenyl)acetic acid;
(R)-2-(2-((3′-(1-aminoethyl)-5-((cyclopropylmethyl)(methyl)amino)-2′-fluoro-[1,1′-biphenyl]-3-yl)methoxy)phenyl)acetic acid;
(R)-2-(2-((3′-(1-aminoethyl)-2′-fluoro-5-((tetrahydro-2H-pyran-4-yl)methyl)-[1,1′-biphenyl]-3-yl)methoxy)phenyl)acetic acid;
(R)-2-(2-((3′-(1-aminoethyl)-5-(azetidin-1-yl)-2′-fluoro-[1,1′-biphenyl]-3-yl)methoxy)phenyl)acetic acid;
(R)-2-(2-((3′-(1-aminoethyl)-2′-fluoro-5-(2-(tetrahydro-2H-pyran-4-y)ethyl)-[1,1′-biphenyl]-3-yl)methoxy)phenyl)acetic acid;
Ethyl 2-(2-((3′-((R)-1-aminoethyl)-2′-fluoro-5-((((R)-tetrahydrofuran-2-yl)methyl)amino)-[1,1′-biphenyl]-3-yl)methoxy)phenyl)acetate;
(R)-2-(2-((3′-(1-aminoethyl)-2′-fluoro-5-(methylsulfonyl)-[1,1′-biphenyl]-3-yl)methoxy)phenyl)acetic acid;
(R)-2-(2-((3′-(1-aminoethyl)-5-(methylsulfonyl)-[1,1′-biphenyl]-3-yl)methoxy)phenyl)acetic acid;
(S)-2-(2-((3′-(1-aminoethyl)-2′-fluoro-5-(methylsulfonyl)-[1,1′-biphenyl]-3-yl)methoxy)phenyl)acetic acid;
(R)-2-(2-((6-(3-(1-Aminoethyl)-2-fluorophenyl)benzo[d][1,3]dioxol-4-yl)methoxy)phenyl)acetic acid;
(R)-2-(2-((6-(3-(1-aminoethyl)phenyl)benzo[d][1,3]dioxol-4-yl)methoxy)phenyl)acetic acid;
(S)-2-(2-((6-(3-(1-amino-2-hydroxyethyl)phenyl)benzo[d][1,3]dioxol-4-yl)methoxy)phenyl)acetic acid;
(R)-2-(2-((3′-(1-Aminoethyl)-5-ethoxy-2′-fluoro-[1,1′-biphenyl]-3-yl)methoxy)phenyl)acetic acid;
(R)-2-(2-((3′-(1-aminoethyl)-5-ethoxy-[1,1′-biphenyl]-3-yl)methoxy)phenyl)acetic acid;
2-(2-((3′-(1-amino-2-fluoroethyl)-5-ethoxy-2′-fluoro-[1,1′-biphenyl]-3-yl)methoxy)phenyl)acetic acid;
(R)-2-(2-((3′-(1-aminoethyl)-2′-fluoro-5-((1-methylcyclopropyl)methoxy)-[1,1′-biphenyl]-3-yl)methoxy)phenyl)acetic acid;
(S)-2-(2-((3′-(1-aminoethyl)-2′-fluoro-5-((1-methylcyclopropyl)methoxy)-[1,1′-biphenyl]-3-yl)methoxy)phenyl)acetic acid;
(R)-2-(2-((3′-(1-aminoethyl)-5-((1-methylcyclopropyl)methoxy)-[1,1′-biphenyl]-3-yl)methoxy)phenyl)acetic acid;
(R)-2-(2-((3′-(1-aminoethyl)-2′-fluoro-5-((3-methyloxetan-3-yl)methoxy)-[1,1′-biphenyl]-3-yl)methoxy)phenyl)acetic acid;
(R)-2-(2-((3′-(1-aminoethyl)-5-((3-methyloxetan-3-yl)methoxy)-[1,1′-biphenyl]-3-yl)methoxy)phenyl)acetic acid;
2-(2-((3′-((R)-1-aminoethyl)-2′-fluoro-5-((tetrahydro-2H-pyran-2-yl)methoxy)-[1,1′-biphenyl]-3-yl)methoxy)phenyl)acetic acid;
2-(2-((3′-((R)-1-aminoethyl)-5-((tetrahydro-2H-pyran-2-yl)methoxy)-[1,1′-biphenyl]-3-yl)methoxy)phenyl)acetic acid;
2-(2-((3′-((R)-1-aminoethyl)-2′-fluoro-5-(pyrrolidin-2-ylmethoxy)-[1,1′-biphenyl]-3-yl)methoxy)phenyl)acetic acid;
2-(2-((3′-((R)-1-aminoethyl)-5-(pyrrolidin-2-ylmethoxy)-[1,1′-biphenyl]-3-yl)methoxy)phenyl)acetic acid;
(S)-Ethyl 2-(2-((3′-(1-amino-2-hydroxyethyl)-[1,1′-biphenyl]-3-yl)methoxy)phenyl)acetate;
(R)-2-(2-((3′-(1-aminoethyl)-2′-fluoro-5-(3-fluoropropyl)-[1,1′-biphenyl]-3-yl)methoxy)phenyl)acetic acid;
(S)-2-(2-((3′-(1-aminoethyl)-2′-fluoro-5-(3-fluoropropyl)-[1,1′-biphenyl]-3-yl)methoxy)phenyl)acetic acid;
(R)-2-(2-((3′-(1-aminoethyl)-5-(3-fluoropropyl)-[1,1′-biphenyl]-3-yl)methoxy)phenyl)acetic acid;
(S)-2-(2-((3′-(1-amino-2-hydroxyethyl)-5-(3-fluoropropyl)-[1,1′-biphenyl]-3-yl)methoxy)phenyl)acetic acid;
(R)-2-(2-((3′-(1-aminoethyl)-2′-fluoro-4-methyl-[1,1′-biphenyl]-3-yl)methoxy)phenyl)acetic acid;
(S)-2-(2-((3′-(1-aminoethyl)-2′-fluoro-4-methyl-[1,1′-biphenyl]-3-yl)methoxy)phenyl)acetic acid;
(R)-2-(2-((3′-(1-aminoethyl)-4-chloro-2′-fluoro-[1,1′-biphenyl]-3-yl)methoxy)phenyl)acetic acid;
(R)-2-(2-((3′-(1-aminoethyl)-2′-fluoro-4-methoxy-[1,1′-biphenyl]-3-yl)methoxy)phenyl)acetic acid;
(S)-2-(2-((3′-(1-Amino-2-hydroxyethyl)-5′-chloro-[1,1′-biphenyl]-3-yl)methoxy)phenyl)acetic acid;
2-(2-((3′-(1-amino-3-fluoropropyl)-[1,1′-biphenyl]-3-yl)methoxy)phenyl)acetic acid;
(R)-2-(2-((3′-(1-amino-3-fluoropropyl)-5-chloro-[1,1′-biphenyl]-3-yl)methoxy)phenyl)acetic acid;
(R)-2-(2-((3′-(1-aminoethyl)-4-cyclopropyl-2′-fluoro-[1,1′-biphenyl]-3-yl)methoxy)phenyl)acetic acid;
2-(2-((3′-((R)-1-Aminoethyl)-5-(((2,2-difluorocyclopropyl)methyl)amino)-2′-fluoro-[1,1′-biphenyl]-3-yl)methoxy)phenyl)acetic acid;
(R)-2-(2-((3′-(1-aminoethyl)-4-ethyl-2′-fluoro-[1,1′-biphenyl]-3-yl)methoxy)phenyl)acetic acid;
(R)-2-(2-((3′-(1-aminoethyl)-2′-fluoro-4,5-dimethyl-[1,1′-biphenyl]-3-yl)methoxy)phenyl)acetic acid;
(S)-2-(2-((3′-(1-amino-2-fluoroethyl)-5-(((2,2,2-trifluoroethyl)amino)methyl)-[1,1′-biphenyl]-3-yl)methoxy)phenyl)acetic acid;
2-(2-((3′-(1-amino-2-fluoroethyl)-2′-fluoro-5-(((2,2,2-trifluoroethyl)amino)methyl)-[1,1′-biphenyl]-3-yl)methoxy)phenyl)acetic acid;
(R)-2-(2-((3′-(1-aminoethyl)-2′-fluoro-5-(((2,2,2-trifluoroethyl)amino)methyl)-[1,1′-biphenyl]-3-yl)methoxy)phenyl)acetic acid;
2-(2-((3′-(aminomethyl)-5-(((2,2,2-trifluoro-1-phenylethyl)amino)methyl)-[1,1′-biphenyl]-3-yl)methoxy)phenyl)acetic acid;
2-(2-((3′-((S)-1-amino-2-hydroxyethyl)-5-((S)-chroman-4-ylcarbamoyl)-[1,1′-biphenyl]-3-yl)methoxy)phenyl)acetic acid;
2-(2-((3′-((methylamino)methyl)-[1,1′-biphenyl]-3-yl)methoxy)phenyl)acetic acid;
2-(2-((3′-(aminomethyl)-6-cyano-[1,1′-biphenyl]-3-yl)methoxy)phenyl)acetic acid;
2-(2-((3′-(aminomethyl)-[1,1′-biphenyl]-3-yl)methoxy)phenyl)propanoic acid;
2-(2-((3′-(aminomethyl)-5′-chloro-[1,1′-biphenyl]-3-yl)methoxy)phenyl)acetic acid;
2-(2-((3′-(aminomethyl)-5′-methyl-[1,1′-biphenyl]-3-yl)methoxy)phenyl)acetic acid;
2-(2-((3′-(aminomethyl)-[1,1′-biphenyl]-3-yl)methoxy)-4-ethylphenyl)acetic acid;
2-(2-((3′-(aminomethyl)-[1,1′-biphenyl]-3-yl)methoxy)-4-cyclopropylphenyl)acetic acid;
2-(2-((3′-(aminomethyl)-[1,1′-biphenyl]-3-yl)methoxy)-4-cyanophenyl)acetic acid2-(2-((3′-(aminomethyl)-[1,1′-biphenyl]-3-yl)methoxy)-4-carbamoylphenyl)acetic acid;
2-(4-(acetamidomethyl)-2-((3′-(aminomethyl)-[1,1′-biphenyl]-3-yl)methoxy)phenyl)acetic acid;
2-(2-((3′-(aminomethyl)-[1,1′-biphenyl]-3-yl)methoxy)-4-(ethylamino)phenyl)acetic acid;
2-(2-((3′-(aminomethyl)-[1,1′-biphenyl]-3-yl)methoxy)-5-cyanophenyl)acetic acid;
2-(2-((3′-(aminomethyl)-[1,1′-biphenyl]-3-yl)methoxy)-5-carbamoylphenyl)acetic acid;
2-(2-((3′-(aminomethyl)-[1,1′-biphenyl]-3-yl)methoxy)-4-(cyclopropylmethyl)phenyl)acetic acid;
2-acetamido-2-(2-((3′-(aminomethyl)-[1,1′-biphenyl]-3-yl)methoxy)phenyl)acetic acid; and
2-(2-((3′-(aminomethyl)-[1,1′-biphenyl]-3-yl)methoxy)phenyl)-2-(2-phenylcyclopropanecarboxamido)acetate.

In a twenty seventh embodiment, pharmaceutical compositions are provided which comprise one or more pharmaceutically acceptable carriers and a therapeutically effective amount of a compound of any one of embodiments one to twenty six.

In a twenty eighth embodiment, a combination, in particular a pharmaceutical combination, comprising a therapeutically effective amount of the compound according to any one of embodiments one to twenty six and a second therapeutically active agent.

Some of the compounds listed supra have been prepared in enantiopure form (i.e., greater than about 80%, greater than 90% or greater than 95% enantiomeric purity). Other compounds have been isolated as mixtures of stereoisomers, e.g., diastereoisomeric mixtures of two or more diastereoisomers. Each compound isolated as a mixture of stereoisomers has been marked as mixture in the foregoing list.

In one embodiment, the invention provides a combination, in particular a pharmaceutical combination, comprising a therapeutically effective amount of the compound according to the definition of formula (I), (11), (Ill), (IV) or subformulae thereof or any one of the specifically disclosed compounds of the invention and one or more therapeutically active agents (preferably selected from those listed infra).

For purposes of interpreting this specification, the following definitions will apply and whenever appropriate, terms used in the singular will also include the plural and vice versa.

As used herein, the term “alkyl” refers to a fully saturated branched or unbranched hydrocarbon moiety having up to 20 carbon atoms. Unless otherwise provided, alkyl refers to hydrocarbon moieties having 1 to 16 carbon atoms, 1 to 10 carbon atoms, 1 to 7 carbon atoms, or 1 to 4 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl and the like.

As used herein, the term “alkylene” refers to divalent alkyl group as defined herein above having 1 to 20 carbon atoms. It comprises 1 to 20 carbon atoms, Unless otherwise provided, alkylene refers to moieties having 1 to 16 carbon atoms, 1 to 10 carbon atoms, 1 to 7 carbon atoms, or 1 to 4 carbon atoms. Representative examples of alkylene include, but are not limited to, methylene, ethylene, n-propylene, iso-propylene, n-butylene, sec-butylene, iso-butylene, tert-butylene, n-pentylene, isopentylene, neopentylene, n-hexylene, 3-methylhexylene, 2,2-dimethylpentylene, 2,3-dimethylpentylene, n-heptylene, n-octylene, n-nonylene, n-decylene and the like.

As used herein, the term “haloalkyl” refers to an alkyl as defined herein, that is substituted by one or more halo groups as defined herein. The haloalkyl can be monohaloalkyl, dihaloalkyl or polyhaloalkyl including perhaloalkyl. A monohaloalkyl can have one iodo, bromo, chloro or fluoro within the alkyl group. Dihaloalkyl and polyhaloalkyl groups can have two or more of the same halo atoms or a combination of different halo groups within the alkyl. Typically the polyhaloalkyl contains up to 12, or 10, or 8, or 6, or 4, or 3, or 2 halo groups. Non-limiting examples of haloalkyl include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. A perhaloalkyl refers to an alkyl having all hydrogen atoms replaced with halo atoms.

The term “aryl” refers to an aromatic hydrocarbon group having 6-20 carbon atoms in the ring portion. Typically, aryl is monocyclic, bicyclic or tricyclic aryl having 6-20 carbon atoms.

Furthermore, the term “aryl” as used herein, refers to an aromatic substituent which can be a single aromatic ring, or multiple aromatic rings that are fused together.

Non-limiting examples include phenyl, naphthyl or tetrahydronaphthyl, each of which may optionally be substituted by 1-4 substituents, such as alkyl, trifluoromethyl, cycloalkyl, halogen, hydroxy, alkoxy, acyl, alkyl-C(O)—O—, aryl-O—, heteroaryl-O—, amino, thiol, alkyl-S—, aryl-S—, nitro, cyano, carboxy, alkyl-O—C(O)—, carbamoyl, alkyl-S(O)—, sulfonyl, sulfonamido, phenyl, and heterocyclyl.

As used herein, the term “alkoxy” refers to alkyl-O—, wherein alkyl is defined herein above. Representative examples of alkoxy include, but are not limited to, methoxy, ethoxy, propoxy, 2-propoxy, butoxy, tert-butoxy, pentyloxy, hexyloxy, cyclopropyloxy-, cyclohexyloxy- and the like. Typically, alkoxy groups have about 1-7, more preferably about 1-4 carbons.

As used herein, the term “heterocyclyl” or “heterocyclo” refers to a saturated or unsaturated non-aromatic ring or ring system, e.g., which is a 4-, 5-, 6-, or 7-membered monocyclic, 7-, 8-, 9-, 10-, 11-, or 12-membered bicyclic or 10-, 11-, 12-, 13-, 14- or 15-membered tricyclic ring system and contains at least one heteroatom selected from O, S and N, where the N and S can also optionally be oxidized to various oxidation states. The heterocyclic group can be attached at a heteroatom or a carbon atom. The heterocyclyl can include fused or bridged rings as well as spirocyclic rings. Examples of heterocycles include tetrahydrofuran (THF), dihydrofuran, 1,4-dioxane, morpholine, 1,4-dithiane, piperazine, piperidine, 1,3-dioxolane, imidazolidine, imidazoline, pyrroline, pyrrolidine, tetrahydropyran, dihydropyran, oxathiolane, dithiolane, 1,3-dioxane, 1,3-dithiane, oxathiane, thiomorpholine, and the like.

The term “heterocyclyl” further refers to heterocyclic groups as defined herein substituted with 1 to 5 substituents independently selected from the groups consisting of the following:

(a) alkyl;

(b) hydroxy (or protected hydroxy);

(c) halo;

(d) oxo, i.e., ═O;

(e) amino, alkylamino or dialkylamino;

(f) alkoxy;

(g) cycloalkyl;

(h) carboxyl;

(i) heterocyclooxy, wherein heterocyclooxy denotes a heterocyclic group bonded through an oxygen bridge;

(j) alkyl-O—C(O)—;

(k) mercapto;

(l) nitro;

(m) cyano;

(n) sulfamoyl or sulfonamido;

(o) aryl;

(p) alkyl-C(O)—O—;

(q) aryl-C(O)—O—;

(r) aryl-S—;

(s) aryloxy;

(t) alkyl-S—;

(u) formyl, i.e., HC(O)—;

(v) carbamoyl;

(w) aryl-alkyl-; and

(x) aryl substituted with alkyl, cycloalkyl, alkoxy, hydroxy, amino, alkyl-C(O)—NH—, alkylamino, dialkylamino or halogen.

As used herein, the term “cycloalkyl” refers to saturated or unsaturated monocyclic, bicyclic or tricyclic hydrocarbon groups of 3-12 carbon atoms. Unless otherwise provided, cycloalkyl refers to cyclic hydrocarbon groups having between 3 and 9 ring carbon atoms or between 3 and 7 ring carbon atoms, each of which can be optionally substituted by one, or two, or three, or more substituents independently selected from the group consisting of alkyl, halo, oxo, hydroxy, alkoxy, alkyl-C(O)—, acylamino, carbamoyl, alkyl-NH—, (alkyl)₂N—, thiol, alkyl-S—, nitro, cyano, carboxy, alkyl-O—C(O)—, sulfonyl, sulfonamido, sulfamoyl, and heterocyclyl. Exemplary monocyclic hydrocarbon groups include, but are not limited to, cyclopropyl, cyclobutyl, cyclopentyl, cyclopentenyl, cyclohexyl and cyclohexenyl and the like. Exemplary bicyclic hydrocarbon groups include bornyl, indyl, hexahydroindyl, tetrahydronaphthyl, decahydronaphthyl, bicyclo[2.1.1]hexyl, bicyclo[2.2.1]heptyl, bicyclo[2.2.1]heptenyl, 6,6-dimethylbicyclo[31.1]heptyl, 2,6,6-trimethylbicyclo[3.1.1]heptyl, bicyclo[2.2.2]octyl and the like. Exemplary tricyclic hydrocarbon groups include adamantyl and the like.

As used herein, the term “aryloxy” refers to both an —O-aryl and an —O-heteroaryl group, wherein aryl and heteroaryl are defined herein.

As used herein, the term “heteroaryl” refers to a 5-14 membered monocyclic- or bicyclic- or tricyclic-aromatic ring system, having 1 to 8 heteroatoms selected from N, O or S. Typically, the heteroaryl is a 5-10 membered ring system (e.g., 5-7 membered monocycle or an 8-10 membered bicycle) or a 5-7 membered ring system. Typical heteroaryl groups include 2- or 3-thienyl, 2- or 3-furyl, 2- or 3-pyrrolyl, 2-, 4-, or 5-imidazolyl, 3-, 4-, or 5-pyrazolyl, 2-, 4-, or 5-thiazolyl, 3-, 4-, or 5-isothiazolyl, 2-, 4-, or 5-oxazolyl, 3-, 4-, or 5-isoxazolyl, 3- or 5-1,2,4-triazolyl, 4- or 5-1,2,3-triazolyl, tetrazolyl, 2-, 3-, or 4-pyridyl, 3- or 4-pyridazinyl, 3-, 4-, or 5-pyrazinyl, 2-pyrazinyl, and 2-, 4-, or 5-pyrimidinyl.

A heteroaryl group may be substituted with 1 to 5 substituents independently selected from the groups consisting of the following:

(a) alkyl;

(b) hydroxy (or protected hydroxy);

(c) halo;

(d) oxo, i.e., ═O;

(e) amino, alkylamino or dialkylamino;

(f) alkoxy;

(g) cycloalkyl;

(h) carboxyl;

(i) heterocyclooxy, wherein heterocyclooxy denotes a heterocyclic group bonded through an oxygen bridge;

(j) alkyl-O—C(O)—;

(k) mercapto;

(l) nitro;

(m) cyano;

(n) sulfamoyl or sulfonamido;

(o) aryl;

(p) alkyl-C(O)—O—;

(q) aryl-C(O)—O—;

(r) aryl-S—;

(s) aryloxy;

(t) alkyl-S—;

(u) formyl, i.e., HC(O)—;

(v) carbamoyl;

(w) aryl-alkyl-; and

(x) aryl substituted with alkyl, cycloalkyl, alkoxy, hydroxy, amino, alkyl-C(O)—NH—, alkylamino, dialkylamino or halogen.

As used herein, the term “halogen” or “halo” refers to fluoro, chloro, bromo, and iodo.

As used herein, the term “optionally substituted” unless otherwise specified refers to a group that is unsubstituted or is substituted by one or more, typically 1, 2, 3 or 4, suitable non-hydrogen substituents, each of which is independently selected from the group consisting of:

(a) alkyl;

(b) hydroxy (or protected hydroxy);

(c) halo;

(d) oxo, i.e., ═O;

(e) amino, alkylamino or dialkylamino;

(f) alkoxy;

(g) cycloalkyl;

(h) carboxyl;

(i) heterocyclooxy, wherein heterocyclooxy denotes a heterocyclic group bonded through an oxygen bridge;

(j) alkyl-O—C(O)—;

(k) mercapto;

(l) nitro;

(m) cyano;

(n) sulfamoyl or sulfonamido;

(o) aryl;

(p) alkyl-C(O)—O—;

(q) aryl-C(O)—O—;

(r) aryl-S—;

(s) aryloxy;

(t) alkyl-S—;

(u) formyl, i.e., HC(O)—;

(v) carbamoyl;

(w) aryl-alkyl-; and

(x) aryl substituted with alkyl, cycloalkyl, alkoxy, hydroxy, amino, alkyl-C(O)—NH—, alkylamino, dialkylamino or halogen.

As used herein, the term “isomers” refers to different compounds that have the same molecular formula but differ in arrangement and configuration of the atoms. Also as used herein, the term “an optical isomer” or “a stereoisomer” refers to any of the various stereo isomeric configurations which may exist for a given compound of the present invention and includes geometric isomers. It is understood that a substituent may be attached at a chiral center of a carbon atom. Therefore, the invention includes enantiomers, diastereomers or racemates of the compound. “Enantiomers” are a pair of stereoisomers that are non-superimposable mirror images of each other. A 1:1 mixture of a pair of enantiomers is a “racemic” mixture. “Diastereoisomers” or “diastereomers” are stereoisomers that have at least two asymmetric atoms, but which are not mirror-images of each other. The absolute stereochemistry is specified according to the Cahn-Ingold-Prelog R-S system. When a compound is a pure enantiomer the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line or as entantiomer 1 or 2 (or diastereomer 1 or 2) depending on elution time by chiral chromatography. Certain of the compounds described herein contain one or more asymmetric centers or axes and may thus give rise to enantiomers, diastereomers, and other stereoisomeric forms that may be defined, in terms of absolute stereochemistry, as (R)- or (S)-. The present invention is meant to include all such possible isomers, including racemic mixtures, optically pure forms and intermediate mixtures. Optically active (R)- and (S)- isomers may be prepared using chiral synthons or chiral reagents, or resolved using conventional techniques. If the compound contains a double bond, the substituent may be E or Z configuration. If the compound contains a disubstituted cycloalkyl, the cycloalkyl substituent may have a cis- or trans-configuration. All tautomeric forms are also intended to be included.

As used herein, the term “pharmaceutically acceptable salts” refers to salts that retain the biological effectiveness and properties of the compounds of this invention and, which typically are not biologically or otherwise undesirable. In many cases, the compounds of the present invention are capable of forming acid and/or base salts by virtue of the presence of amino and/or carboxyl groups or groups similar thereto.

Pharmaceutically acceptable acid addition salts can be formed with inorganic acids and organic acids, e.g., acetate, aspartate, benzoate, besylate, bromide/hydrobromide, bicarbonate/carbonate, bisulfate/sulfate, camphorsulfornate, chloride/hydrochloride, chlortheophyllonate, citrate, ethandisulfonate, fumarate, gluceptate, gluconate, glucuronate, hippurate, hydroiodide/iodide, isethionate, lactate, lactobionate, laurylsulfate, malate, maleate, malonate, mandelate, mesylate, methylsulphate, naphthoate, napsylate, nicotinate, nitrate, octadecanoate, oleate, oxalate, palmitate, pamoate, phosphate/hydrogen phosphate/dihydrogen phosphate, polygalacturonate, propionate, stearate, succinate, sulfosalicylate, tartrate, tosylate and trifluoroacetate salts. Inorganic acids from which salts can be derived include, for example, hydrochloric acid, hydrobromic acid, sulfuric acid, nitric acid, and phosphoric acid. Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like.

Pharmaceutically acceptable base addition salts can be formed with inorganic and organic bases. Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

In another aspect, the present invention provides (R)-2-(2-((3′-(1-Aminoethyl)-5-(hydroxymethyl)-[1,1′-biphenyl]-3-yl)methoxy)phenyl)acetic acid in acetate, ascorbate, adipate, aspartate, benzoate, besylate, bromide/hydrobromide, bicarbonate/carbonate, bisulfate/sulfate, camphorsulfonate, caprate, chloride/hydrochloride, chlortheophyllonate, citrate, ethandisulfonate, fumarate, gluceptate, gluconate, glucuronate, glutamate, glutarate, glycolate, hippurate, hydroiodide/iodide, isethionate, lactate, lactobionate, laurylsulfate, malate, maleate, malonate, mandelate, mesylate, methylsulphate, mucate, naphthoate, napsylate, nicotinate, nitrate, octadecanoate, oleate, oxalate, palmitate, pamoate, phosphate/hydrogen phosphate/dihydrogen phosphate, polygalacturonate, propionate, sebacate, stearate, succinate, sulfosalicylate, sulfate, tartrate, tosylate trifenatate, trifluoroacetate or xinafoate salt form. In yet another aspect, the present invention provides compounds of formula I in C₁-C₄alkyl sufonic acid, benzenesulfonic acid or mono-, di- or tri- C₁-C₄alkyl substituted benzene sufonic acid addition salt form.

In another aspect, the present invention provides (S)-2-(2-((3′-(1-Amino-2-hydroxyethyl)-[1,1′-biphenyl]-3-yl)methoxy)phenyl)acetic acid in acetate, ascorbate, adipate, aspartate, benzoate, besylate, bromide/hydrobromide, bicarbonate/carbonate, bisulfate/sulfate, camphorsulfonate, caprate, chloride/hydrochloride, chlortheophyllonate, citrate, ethandisulfonate, fumarate, gluceptate, gluconate, glucuronate, glutamate, glutarate, glycolate, hippurate, hydroiodide/iodide, isethionate, lactate, lactobionate, laurylsulfate, malate, maleate, malonate, mandelate, mesylate, methylsulphate, mucate, naphthoate, napsylate, nicotinate, nitrate, octadecanoate, oleate, oxalate, palmitate, pamoate, phosphate/hydrogen phosphate/dihydrogen phosphate, polygalacturonate, propionate, sebacate, stearate, succinate, sulfosalicylate, sulfate, tartrate, tosylate trifenatate, trifluoroacetate or xinafoate salt form. In yet another aspect, the present invention provides compounds of formula I in C₁-C₄alkyl sufonic acid, benzenesulfonic acid or mono-, di- or tri- C₁-C₄alkyl substituted benzene sufonic acid addition salt form.

In another aspect, the present invention provides (S)-2-(2-((3′-(1-Amino-2-hydroxyethyl)-5-ethoxy-[1,1′-biphenyl]-3-yl)methoxy)phenyl)acetic acid in acetate, ascorbate, adipate, aspartate, benzoate, besylate, bromide/hydrobromide, bicarbonate/carbonate, bisulfate/sulfate, camphorsulfonate, caprate, chloride/hydrochloride, chlortheophyllonate, citrate, ethandisulfonate, fumarate, gluceptate, gluconate, glucuronate, glutamate, glutarate, glycolate, hippurate, hydroiodide/iodide, isethionate, lactate, lactobionate, laurylsulfate, malate, maleate, malonate, mandelate, mesylate, methylsulphate, mucate, naphthoate, napsylate, nicotinate, nitrate, octadecanoate, oleate, oxalate, palmitate, pamoate, phosphate/hydrogen phosphate/dihydrogen phosphate, polygalacturonate, propionate, sebacate, stearate, succinate, sulfosalicylate, sulfate, tartrate, tosylate trifenatate, trifluoroacetate or xinafoate salt form. In yet another aspect, the present invention provides compounds of formula I in C₁-C₄alkyl sufonic acid, benzenesulfonic acid or mono-, di- or tri- C₁-C₄alkyl substituted benzene sufonic acid addition salt form.

In another aspect, the present invention provides (S)-2-(2-((3′-(1-Amino-2-hydroxyethyl)-5-(2-hydroxypropan-2-yl)-[1,1′-biphenyl]-3-yl)methoxy)phenyl)acetic acid in acetate, ascorbate, adipate, aspartate, benzoate, besylate, bromide/hydrobromide, bicarbonate/carbonate, bisulfate/sulfate, camphorsulfonate, caprate, chloride/hydrochloride, chlortheophyllonate, citrate, ethandisulfonate, fumarate, gluceptate, gluconate, glucuronate, glutamate, glutarate, glycolate, hippurate, hydroiodide/iodide, isethionate, lactate, lactobionate, laurylsulfate, malate, maleate, malonate, mandelate, mesylate, methylsulphate, mucate, naphthoate, napsylate, nicotinate, nitrate, octadecanoate, oleate, oxalate, palmitate, pamoate, phosphate/hydrogen phosphate/dihydrogen phosphate, polygalacturonate, propionate, sebacate, stearate, succinate, sulfosalicylate, sulfate, tartrate, tosylate trifenatate, trifluoroacetate or xinafoate salt form. In yet another aspect, the present invention provides compounds of formula I in C₁-C₄alkyl sufonic acid, benzenesulfonic acid or mono-, di- or tri- C₁-C₄alkyl substituted benzene sufonic acid addition salt form.

In another aspect, the present invention provides (R)-2-(2-((3′-(1-Aminoethyl)-2′-fluoro-5-(methoxymethyl)-[1,1′-biphenyl]-3-yl)methoxy)phenyl)acetic acid in acetate, ascorbate, adipate, aspartate, benzoate, besylate, bromide/hydrobromide, bicarbonate/carbonate, bisulfate/sulfate, camphorsulfonate, caprate, chloride/hydrochloride, chlortheophyllonate, citrate, ethandisulfonate, fumarate, gluceptate, gluconate, glucuronate, glutamate, glutarate, glycolate, hippurate, hydroiodide/iodide, isethionate, lactate, lactobionate, laurylsulfate, malate, maleate, malonate, mandelate, mesylate, methylsulphate, mucate, naphthoate, napsylate, nicotinate, nitrate, octadecanoate, oleate, oxalate, palmitate, pamoate, phosphate/hydrogen phosphate/dihydrogen phosphate, polygalacturonate, propionate, sebacate, stearate, succinate, sulfosalicylate, sulfate, tartrate, tosylate trifenatate, trifluoroacetate or xinafoate salt form. In yet another aspect, the present invention provides compounds of formula I in C₁-C₄alkyl sufonic acid, benzenesulfonic acid or mono-, di- or tri- C₁-C₄alkyl substituted benzene sufonic acid addition salt form.

In another aspect, the present invention provides (R)-2-(2-((3′-(1-Aminoethyl)-5-(methoxymethyl)-[1,1′-biphenyl]-3-yl)methoxy)phenyl)acetic acid in acetate, ascorbate, adipate, aspartate, benzoate, besylate, bromide/hydrobromide, bicarbonate/carbonate, bisulfate/sulfate, camphorsulfonate, caprate, chloride/hydrochloride, chlortheophyllonate, citrate, ethandisulfonate, fumarate, gluceptate, gluconate, glucuronate, glutamate, glutarate, glycolate, hippurate, hydroiodide/iodide, isethionate, lactate, lactobionate, laurylsulfate, malate, maleate, malonate, mandelate, mesylate, methylsulphate, mucate, naphthoate, napsylate, nicotinate, nitrate, octadecanoate, oleate, oxalate, palmitate, pamoate, phosphate/hydrogen phosphate/dihydrogen phosphate, polygalacturonate, propionate, sebacate, stearate, succinate, sulfosalicylate, sulfate, tartrate, tosylate trifenatate, trifluoroacetate or xinafoate salt form. In yet another aspect, the present invention provides compounds of formula I in C₁-C₄alkyl sufonic acid, benzenesulfonic acid or mono-, di- or tri- C₁-C₄alkyl substituted benzene sufonic acid addition salt form.

In another aspect, the present invention provides (S)-2-(2-((3′-(1-Amino-2-hydroxyethyl)-5-((cyclopropylmethyl)amino)-[1,1′-biphenyl]-3-yl)methoxy)phenyl)acetic acid in acetate, ascorbate, adipate, aspartate, benzoate, besylate, bromide/hydrobromide, bicarbonate/carbonate, bisulfate/sulfate, camphorsulfonate, caprate, chloride/hydrochloride, chlortheophyllonate, citrate, ethandisulfonate, fumarate, gluceptate, gluconate, glucuronate, glutamate, glutarate, glycolate, hippurate, hydroiodide/iodide, isethionate, lactate, lactobionate, laurylsulfate, malate, maleate, malonate, mandelate, mesylate, methylsulphate, mucate, naphthoate, napsylate, nicotinate, nitrate, octadecanoate, oleate, oxalate, palmitate, pamoate, phosphate/hydrogen phosphate/dihydrogen phosphate, polygalacturonate, propionate, sebacate, stearate, succinate, sulfosalicylate, sulfate, tartrate, tosylate trifenatate, trifluoroacetate or xinafoate salt form. In yet another aspect, the present invention provides compounds of formula I in C₁-C₄alkyl sufonic acid, benzenesulfonic acid or mono-, di- or tri- C₁-C₄alkyl substituted benzene sufonic acid addition salt form.

In another aspect, the present invention provides (S)-2-(2-((3′-(Aminomethyl)-5-(((tetrahydrofuran-2-yl)methyl)amino)-[1,1′-biphenyl]-3-yl)methoxy)phenyl)acetic acid in acetate, ascorbate, adipate, aspartate, benzoate, besylate, bromide/hydrobromide, bicarbonate/carbonate, bisulfate/sulfate, camphorsulfonate, caprate, chloride/hydrochloride, chlortheophyllonate, citrate, ethandisulfonate, fumarate, gluceptate, gluconate, glucuronate, glutamate, glutarate, glycolate, hippurate, hydroiodide/iodide, isethionate, lactate, lactobionate, laurylsulfate, malate, maleate, malonate, mandelate, mesylate, methylsulphate, mucate, naphthoate, napsylate, nicotinate, nitrate, octadecanoate, oleate, oxalate, palmitate, pamoate, phosphate/hydrogen phosphate/dihydrogen phosphate, polygalacturonate, propionate, sebacate, stearate, succinate, sulfosalicylate, sulfate, tartrate, tosylate trifenatate, trifluoroacetate or xinafoate salt form. In yet another aspect, the present invention provides compounds of formula I in C₁-C₄alkyl sufonic acid, benzenesulfonic acid or mono-, di- or tri- C₁-C₄alkyl substituted benzene sufonic acid addition salt form.

In another aspect, the present invention provides (R)-2-(2-((3′-(1-aminoethyl)-5-(ethylamino)-2′-fluoro-[1,1′-biphenyl]-3-yl)methoxy)phenyl)acetic acid in acetate, ascorbate, adipate, aspartate, benzoate, besylate, bromide/hydrobromide, bicarbonate/carbonate, bisulfate/sulfate, camphorsulfonate, caprate, chloride/hydrochloride, chlortheophyllonate, citrate, ethandisulfonate, fumarate, gluceptate, gluconate, glucuronate, glutamate, glutarate, glycolate, hippurate, hydroiodide/iodide, isethionate, lactate, lactobionate, laurylsulfate, malate, maleate, malonate, mandelate, mesylate, methylsulphate, mucate, naphthoate, napsylate, nicotinate, nitrate, octadecanoate, oleate, oxalate, palmitate, pamoate, phosphate/hydrogen phosphate/dihydrogen phosphate, polygalacturonate, propionate, sebacate, stearate, succinate, sulfosalicylate, sulfate, tartrate, tosylate trifenatate, trifluoroacetate or xinafoate salt form. In yet another aspect, the present invention provides compounds of formula I in C₁-C₄alkyl sufonic acid, benzenesulfonic acid or mono-, di- or tri- C₁-C₄alkyl substituted benzene sufonic acid addition salt form.

In another aspect, the present invention provides 2-(2-((6-(3-(Aminomethyl)phenyl)-1H-indazol-4-yl)methoxy)phenyl)acetic acid in acetate, ascorbate, adipate, aspartate, benzoate, besylate, bromide/hydrobromide, bicarbonate/carbonate, bisulfate/sulfate, camphorsulfonate, caprate, chloride/hydrochloride, chlortheophyllonate, citrate, ethandisulfonate, fumarate, gluceptate, gluconate, glucuronate, glutamate, glutarate, glycolate, hippurate, hydroiodide/iodide, isethionate, lactate, lactobionate, laurylsulfate, malate, maleate, malonate, mandelate, mesylate, methylsulphate, mucate, naphthoate, napsylate, nicotinate, nitrate, octadecanoate, oleate, oxalate, palmitate, pamoate, phosphate/hydrogen phosphate/dihydrogen phosphate, polygalacturonate, propionate, sebacate, stearate, succinate, sulfosalicylate, sulfate, tartrate, tosylate trifenatate, trifluoroacetate or xinafoate salt form. In yet another aspect, the present invention provides compounds of formula I in C₁-C₄alkyl sufonic acid, benzenesulfonic acid or mono-, di- or tri- C₁-C₄alkyl substituted benzene sufonic acid addition salt form.

The pharmaceutically acceptable salts of the present invention can be synthesized from a parent compound, a basic or acidic moiety, by conventional chemical methods. Generally, such salts can be prepared by reacting free acid forms of these compounds with a stoichiometric amount of the appropriate base (such as Na, Ca, Mg, or K hydroxide, carbonate, bicarbonate or the like), or by reacting free base forms of these compounds with a stoichiometric amount of the appropriate acid. Such reactions are typically carried out in water or in an organic solvent, or in a mixture of the two. Generally, use of non-aqueous media like ether, ethyl acetate, ethanol, isopropanol, or acetonitrile is desirable, where practicable. Lists of additional suitable salts can be found, e.g., in “Remington's Pharmaceutical Sciences”, 20th ed., Mack Publishing Company, Easton, Pa., (1985); and in “Handbook of Pharmaceutical Salts: Properties, Selection, and Use” by Stahl and Wermuth (Wiley-VCH, Weinheim, Germany, 2002).

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the invention include isotopes of hydrogen, carbon, nitrogen, oxygen, phosphorous, fluorine, and chlorine, such as ²H, ³H, ¹¹C, ¹³C, ¹⁴C, ¹⁵N, ¹⁸F ³¹P, ³²P, ³⁵S, ³⁶Cl, ¹²⁵I respectively. The invention includes various isotopically labeled compounds as defined herein, for example those into which radioactive isotopes, such as ³H, ¹³C, and ¹⁴C, are present. Such isotopically labelled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F or labeled compound may be particularly desirable for PET or SPECT studies. Isotopically labeled compounds of this invention and prodrugs thereof can generally be prepared by carrying out the procedures disclosed in the schemes or in the examples and preparations described below by substituting a readily available isotopically labeled reagent for a non-isotopically labeled reagent.

Further, substitution with heavier isotopes, particularly deuterium (i.e., ²H or D) may afford certain therapeutic advantages resulting from greater metabolic stability, for example increased in vivo half-life or reduced dosage requirements or an improvement in therapeutic index. It is understood that deuterium in this context is regarded as a substituent of a compound of the formula (I). The concentration of such a heavier isotope, specifically deuterium, may be defined by the isotopic enrichment factor. The term “isotopic enrichment factor” as used herein means the ratio between the isotopic abundance and the natural abundance of a specified isotope. If a substituent in a compound of this invention is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

In certain embodiments, selective deuteration of compounds of Formula (I) or formula (II) include deuteration of R⁵, when R⁵ is alkanoyl, e.g., C(O)CD₃. In other embodiments, certain substitutents on the proline ring are selectively deuterated. For example, when any of R⁸ or R⁹ are methyl or methoxy, the alkyl residue is preferably deuterated, e.g., CD₃ or OCD₃. In certain other compounds, when two substituents of the proline ring are combined to form a cyclopropyl ring, the unsubstituted methylene carbon is selectively deuterated. In certain other compounds of Formulae (I), (11), (Ill) or (IV), R¹⁰, R¹¹ and/or R¹² is deuterated alkyl, preferably CD₃.

Isotopically-labeled compounds of formula (I) can generally be prepared by conventional techniques known to those skilled in the art or by processes analogous to those described in the accompanying Examples and Preparations using an appropriate isotopically-labeled reagents in place of the non-labeled reagent previously employed.

The compounds of the present invention may inherently or by design form solvates with solvents (including water). Therefore, it is intended that the invention embrace both solvated and unsolvated forms. The term “solvate” refers to a molecular complex of a compound of the present invention (including salts thereof) with one or more solvent molecules. Such solvent molecules are those commonly used in the pharmaceutical art, which are known to be innocuous to a recipient, e.g., water, ethanol, dimethylsulfoxide, acetone and other common organic solvents. The term “hydrate” refers to a molecular complex comprising a compound of the invention and water. Pharmaceutically acceptable solvates in accordance with the invention include those wherein the solvent of crystallization may be isotopically substituted, e.g. D₂O, d₆-acetone, d₆-DMSO.

Compounds of the invention, i.e. compounds of formula (I) that contain groups capable of acting as donors and/or acceptors for hydrogen bonds may be capable of forming co-crystals with suitable co-crystal formers. These co-crystals may be prepared from compounds of formula (I) by known co-crystal forming procedures. Such procedures include grinding, heating, co-subliming, co-melting, or contacting in solution compounds of formula (I) with the co-crystal former under crystallization conditions and isolating co-crystals thereby formed. Suitable co-crystal formers include those described in WO 2004/078163. Hence the invention further provides co-crystals comprising a compound of formula (I).

As used herein, the term “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, binders, excipients, disintegration agents, lubricants, sweetening agents, flavoring agents, dyes, and the like and combinations thereof, as would be known to those skilled in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated.

The term “a therapeutically effective amount” of a compound of the present invention refers to an amount of the compound of the present invention that will elicit the biological or medical response of a subject, for example, reduction or inhibition of an enzyme or a protein activity, or ameliorate symptoms, alleviate conditions, slow or delay disease progression, or prevent a disease, etc. In one non-limiting embodiment, the term “a therapeutically effective amount” refers to the amount of the compound of the present invention that, when administered to a subject, is effective to (1) at least partially alleviating, inhibiting, preventing and/or ameliorating a condition, or a disorder, or a disease or biological process (e.g., tissue regeneration and reproduction) (i) mediated by Factor D, or (ii) associated with Factor D activity, or (iii) characterized by activity (normal or abnormal) of the complement alternative pathway; or (2) reducing or inhibiting the activity of Factor D; or (3) reducing or inhibiting the expression of Factor D; or (4) reducing or inhibiting activation of the complement system and particularly reducing or inhibiting generation of C3a, iC3b, C5a or the membrane attack complex generated by activation of the complement alternative pathway. In another non-limiting embodiment, the term “a therapeutically effective amount” refers to the amount of the compound of the present invention that, when administered to a cell, or a tissue, or a non-cellular biological material, or a medium, is effective to at least partially reducing or inhibiting the activity of Factor D and/or the complement alternative pathway; or at least partially reducing or inhibiting the expression of Factor D and/or the complement alternative pathway. The meaning of the term “a therapeutically effective amount” as illustrated in the above embodiment for Factor D and/or the complement alternative pathway.

As used herein, the term “subject” refers to an animal. Typically the animal is a mammal. A subject also refers to for example, primates (e.g., humans), cows, sheep, goats, horses, dogs, cats, rabbits, rats, mice, fish, birds and the like. In certain embodiments, the subject is a primate. In yet other embodiments, the subject is a human.

As used herein, the term “inhibit”, “inhibition” or “inhibiting” refers to the reduction or suppression of a given condition, symptom, or disorder, or disease, or a significant decrease in the baseline activity of a biological activity or process.

As used herein, the term “treat”, “treating” or “treatment” of any disease or disorder refers in one embodiment, to ameliorating the disease or disorder (i.e., slowing or arresting or reducing the development of the disease or at least one of the clinical symptoms thereof). In another embodiment “treat”, “treating” or “treatment” refers to alleviating or ameliorating at least one physical parameter including those which may not be discernible by the patient. In yet another embodiment, “treat”, “treating” or “treatment” refers to modulating the disease or disorder, either physically, (e.g., stabilization of a discernible symptom), physiologically, (e.g., stabilization of a physical parameter), or both. In yet another embodiment, “treat”, “treating” or “treatment” refers to preventing or delaying the onset or development or progression of the disease or disorder.

As used herein, a subject is “in need of” a treatment if such subject would benefit biologically, medically or in quality of life from such treatment.

As used herein, the term “a,” “an,” “the” and similar terms used in the context of the present invention (especially in the context of the claims) are to be construed to cover both the singular and plural unless otherwise indicated herein or clearly contradicted by the context.

All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed.

Any asymmetric atom (e.g., carbon or the like) of the compound(s) of the present invention can be present in racemic or enantiomerically enriched, for example the (R)-, (S)- or (R,S)-configuration. In certain embodiments, each asymmetric atom has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess in the (R)- or (S)-configuration. Substituents at atoms with unsaturated bonds may, if possible, be present in cis-(Z)- or trans-(E)- form.

Accordingly, as used herein a compound of the present invention can be in the form of one of the possible isomers, rotamers, atropisomers, tautomers or mixtures thereof, for example, as substantially pure geometric (cis or trans) isomers, diastereomers, optical isomers (antipodes), racemates or mixtures thereof.

Any resulting mixtures of isomers can be separated on the basis of the physicochemical differences of the constituents, into the pure or substantially pure geometric or optical isomers, diastereomers, racemates, for example, by chromatography and/or fractional crystallization.

Any resulting racemates of final products or intermediates can be resolved into the optical antipodes by known methods, e.g., by separation of the diastereomeric salts thereof, obtained with an optically active acid or base, and liberating the optically active acidic or basic compound. In particular, a basic moiety may thus be employed to resolve the compounds of the present invention into their optical antipodes, e.g., by fractional crystallization of a salt formed with an optically active acid, e.g., tartaric acid, dibenzoyl tartaric acid, diacetyl tartaric acid, di-O,O′-p-toluoyl tartaric acid, mandelic acid, malic acid or camphor-10-sulfonic acid. Racemic products can also be resolved by chiral chromatography, e.g., high pressure liquid chromatography (HPLC) using a chiral adsorbent.

Compounds of the present invention are either obtained in the free form, as a salt thereof, or as prodrug derivatives thereof.

When both a basic group and an acid group are present in the same molecule, the compounds of the present invention may also form internal salts, e.g., zwitterionic molecules.

The present invention also provides pro-drugs of the compounds of the present invention that converts in vivo to the compounds of the present invention. A pro-drug is an active or inactive compound that is modified chemically through in vivo physiological action, such as hydrolysis, metabolism and the like, into a compound of this invention following administration of the prodrug to a subject. The suitability and techniques involved in making and using pro-drugs are well known by those skilled in the art. Prodrugs can be conceptually divided into two non-exclusive categories, bioprecursor prodrugs and carrier prodrugs. See The Practice of Medicinal Chemistry, Ch. 31-32 (Ed. Wermuth, Academic Press, San Diego, Calif., 2001). Generally, bioprecursor prodrugs are compounds, which are inactive or have low activity compared to the corresponding active drug compound, that contain one or more protective groups and are converted to an active form by metabolism or solvolysis. Both the active drug form and any released metabolic products should have acceptably low toxicity.

Carrier prodrugs are drug compounds that contain a transport moiety, e.g., that improve uptake and/or localized delivery to a site(s) of action. Desirably for such a carrier prodrug, the linkage between the drug moiety and the transport moiety is a covalent bond, the prodrug is inactive or less active than the drug compound, and any released transport moiety is acceptably non-toxic. For prodrugs where the transport moiety is intended to enhance uptake, typically the release of the transport moiety should be rapid. In other cases, it is desirable to utilize a moiety that provides slow release, e.g., certain polymers or other moieties, such as cyclodextrins. Carrier prodrugs can, for example, be used to improve one or more of the following properties: increased lipophilicity, increased duration of pharmacological effects, increased site-specificity, decreased toxicity and adverse reactions, and/or improvement in drug formulation (e.g., stability, water solubility, suppression of an undesirable organoleptic or physiochemical property). For example, lipophilicity can be increased by esterification of (a) hydroxyl groups with lipophilic carboxylic acids (e.g., a carboxylic acid having at least one lipophilic moiety), or (b) carboxylic acid groups with lipophilic alcohols (e.g., an alcohol having at least one lipophilic moiety, for example aliphatic alcohols).

Exemplary prodrugs are, e.g., esters of free carboxylic acids and S-acyl derivatives of thiols and O-acyl derivatives of alcohols or phenols, wherein acyl has a meaning as defined herein. Suitable prodrugs are often pharmaceutically acceptable ester derivatives convertible by solvolysis under physiological conditions to the parent carboxylic acid, e.g., lower alkyl esters, cycloalkyl esters, lower alkenyl esters, benzyl esters, mono- or di-substituted lower alkyl esters, such as the ω-(amino, mono- or di-lower alkylamino, carboxy, lower alkoxycarbonyl)-lower alkyl esters, the α-(lower alkanoyloxy, lower alkoxycarbonyl or di-lower alkylaminocarbonyl)-lower alkyl esters, such as the pivaloyloxymethyl ester and the like conventionally used in the art. In addition, amines have been masked as arylcarbonyloxymethyl substituted derivatives which are cleaved by esterases in vivo releasing the free drug and formaldehyde (Bundgaard, J. Med. Chem. 2503 (1989)). Moreover, drugs containing an acidic NH group, such as imidazole, imide, indole and the like, have been masked with N-acyloxymethyl groups (Bundgaard, Design of Prodrugs, Elsevier (1985)). Hydroxy groups have been masked as esters and ethers. EP 039,051 (Sloan and Little) discloses Mannich-base hydroxamic acid prodrugs, their preparation and use.

Furthermore, the compounds of the present invention, including their salts, can also be obtained in the form of their hydrates, or include other solvents used for their crystallization.

Within the scope of this text, only a readily removable group that is not a constituent of the particular desired end product of the compounds of the present invention is designated a “protecting group”, unless the context indicates otherwise. The protection of functional groups by such protecting groups, the protecting groups themselves, and their cleavage reactions are described for example in standard reference works, such as J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981, in “Methoden der organischen Chemie” (Methods of Organic Chemistry), Houben Weyl, 4th edition, Volume 15/I, Georg Thieme Verlag, Stuttgart 1974, in H.-D. Jakubke and H. Jeschkeit, “Aminosäuren, Peptide, Proteine” (Amino acids, Peptides, Proteins), Verlag Chemie, Weinheim, Deerfield Beach, and Basel 1982, and in Jochen Lehmann, “Chemie der Kohlenhydrate: Monosaccharide und Derivate” (Chemistry of Carbohydrates: Monosaccharides and Derivatives), Georg Thieme Verlag, Stuttgart 1974. A characteristic of protecting groups is that they can be removed readily (i.e. without the occurrence of undesired secondary reactions) for example by solvolysis, reduction, photolysis or alternatively under physiological conditions (e.g. by enzymatic cleavage).

Salts of compounds of the present invention having at least one salt-forming group may be prepared in a manner known to those skilled in the art. For example, salts of compounds of the present invention having acid groups may be formed, for example, by treating the compounds with metal compounds, such as alkali metal salts of suitable organic carboxylic acids, e.g. the sodium salt of 2-ethylhexanoic acid, with organic alkali metal or alkaline earth metal compounds, such as the corresponding hydroxides, carbonates or hydrogen carbonates, such as sodium or potassium hydroxide, carbonate or hydrogen carbonate, with corresponding calcium compounds or with ammonia or a suitable organic amine, stoichiometric amounts or only a small excess of the salt-forming agent preferably being used. Acid addition salts of compounds of the present invention are obtained in customary manner, e.g. by treating the compounds with an acid or a suitable anion exchange reagent. Internal salts of compounds of the present invention containing acid and basic salt-forming groups, e.g. a free carboxy group and a free amino group, may be formed, e.g. by the neutralization of salts, such as acid addition salts, to the isoelectric point, e.g. with weak bases, or by treatment with ion exchangers.

Salts can be converted into the free compounds in accordance with methods known to those skilled in the art. Metal and ammonium salts can be converted, for example, by treatment with suitable acids, and acid addition salts, for example, by treatment with a suitable basic agent.

Mixtures of isomers obtainable according to the invention can be separated in a manner known to those skilled in the art into the individual isomers; diastereoisomers can be separated, for example, by partitioning between polyphasic solvent mixtures, recrystallization and/or chromatographic separation, for example over silica gel or by e.g. medium pressure liquid chromatography over a reversed phase column, and racemates can be separated, for example, by the formation of salts with optically pure salt-forming reagents and separation of the mixture of diastereoisomers so obtainable, for example by means of fractional crystallization, or by chromatography over optically active column materials.

Intermediates and final products can be worked up and/or purified according to standard methods, e.g. using chromatographic methods, distribution methods, (re-) crystallization, and the like.

The following applies in general to all processes mentioned herein before and hereinafter.

All the above-mentioned process steps can be carried out under reaction conditions that are known to those skilled in the art, including those mentioned specifically, in the absence or, customarily, in the presence of solvents or diluents, including, for example, solvents or diluents that are inert towards the reagents used and dissolve them, in the absence or presence of catalysts, condensation or neutralizing agents, for example ion exchangers, such as cation exchangers, e.g. in the H+ form, depending on the nature of the reaction and/or of the reactants at reduced, normal or elevated temperature, for example in a temperature range of from about −100° C. to about 190° C., including, for example, from approximately −80° C. to approximately 150° C., for example at from −80 to −60° C., at room temperature, at from −20 to 40° C. or at reflux temperature, under atmospheric pressure or in a closed vessel, where appropriate under pressure, and/or in an inert atmosphere, for example under an argon or nitrogen atmosphere.

At all stages of the reactions, mixtures of isomers that are formed can be separated into the individual isomers, for example diastereoisomers or enantiomers, or into any desired mixtures of isomers, for example racemates or mixtures of diastereoisomers, for example analogously to the methods described under “Additional process steps”.

The solvents from which those solvents that are suitable for any particular reaction may be selected include those mentioned specifically or, for example, water, esters, such as lower alkyl-lower alkanoates, for example ethyl acetate, ethers, such as aliphatic ethers, for example diethyl ether, or cyclic ethers, for example tetrahydrofuran or dioxane, liquid aromatic hydrocarbons, such as benzene or toluene, alcohols, such as methanol, ethanol or 1- or 2-propanol, nitriles, such as acetonitrile, halogenated hydrocarbons, such as methylene chloride or chloroform, acid amides, such as dimethylformamide or dimethyl acetamide, bases, such as heterocyclic nitrogen bases, for example pyridine or N-methylpyrrolidin-2-one, carboxylic acid anhydrides, such as lower alkanoic acid anhydrides, for example acetic anhydride, cyclic, linear or branched hydrocarbons, such as cyclohexane, hexane or isopentane, methycyclohexane, or mixtures of those solvents, for example aqueous solutions, unless otherwise indicated in the description of the processes. Such solvent mixtures may also be used in working up, for example by chromatography or partitioning.

The compounds, including their salts, may also be obtained in the form of hydrates, or their crystals may, for example, include the solvent used for crystallization. Different crystalline forms may be present.

The invention relates also to those forms of the process in which a compound obtainable as an intermediate at any stage of the process is used as starting material and the remaining process steps are carried out, or in which a starting material is formed under the reaction conditions or is used in the form of a derivative, for example in a protected form or in the form of a salt, or a compound obtainable by the process according to the invention is produced under the process conditions and processed further in situ.

All starting materials, building blocks, reagents, acids, bases, dehydrating agents, solvents and catalysts utilized to synthesize the compounds of the present invention are either commercially available or can be produced by organic synthesis methods known to one of ordinary skill in the art (Houben-Weyl 4^{th} Ed. 1952, Methods of Organic Synthesis, Thieme, Volume 21).

The invention further includes any variant of the present processes, in which an intermediate product obtainable at any stage thereof is used as starting material and the remaining steps are carried out, or in which the starting materials are formed in situ under the reaction conditions, or in which the reaction components are used in the form of their salts or optically pure materials. Typically, the compounds of formula (I) can be prepared according to the Schemes provided infra. Compounds of the invention and intermediates can also be converted into each other according to methods generally known to those skilled in the art.

Compounds such as S-3, wherein S_{a-d} is independently selected from halo, C₁-C₄alkyl or C₁-C₄alkoxy and hydroxyC₁-C₄alkyl, can be prepared by the general method outlined in Scheme 1.

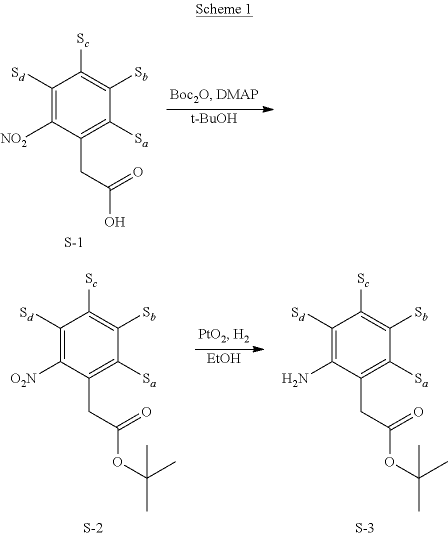

The acid group in S-1 (when, for example, Sₐ is Br, S_{b}, S_{c}, S_{d} are H, CAS #37777-74-5) can be protected as the corresponding tert-butyl ester with treatment of Boc-anhydride and DMAP in t-butanol at room temperature. The nitro group of S-2 can then be reduced under hydrogen atmosphere with platinum oxide as the catalyst in ethanol at room temperature to afford S-3.

Alternatively, compounds such as S-3, wherein S_{a-d} is independently selected from halo, alkyl or alkoxyl can be prepared by another general method outlined in Scheme 2.

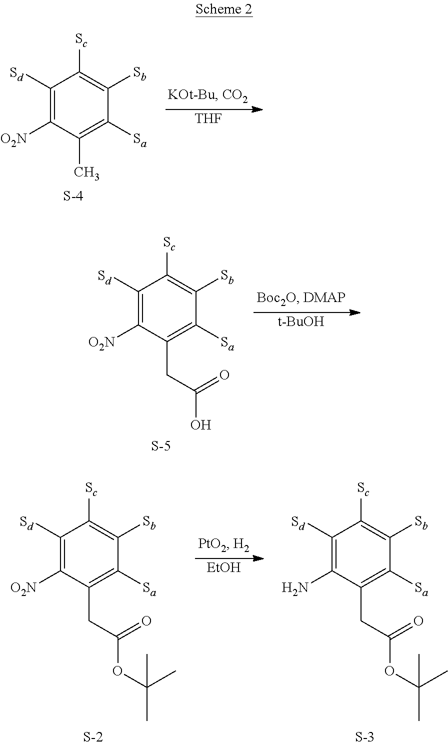

The methyl group of S-4 (when, for example, Sₐ, S_{b}, S_{c} are H, and S_{d} is CH₃, CAS #81-20-9) can be deprotonated with potassium t-butoxide in THF at −78° C. The resulting anion can be trapped with pellets of solid CO₂ to provide the corresponding acid after purification. S-5 can then be transformed to provide S-3 by the same two-step sequence shown in Scheme 1.

Alternatively, compounds such as S-3, wherein S_{a-d} is independently selected from halo, alkyl or alkoxyl can be prepared by another general method outlined in Scheme 3.

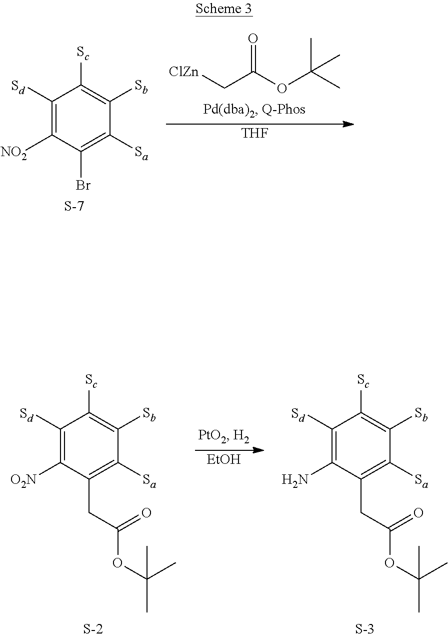

The bromo group of S-7 (when for example Sₐ, S_{b}, S_{c} are H, S_{d} is F, CAS #886762-70-5) can be coupled with a zinc reagent ((2-(tert-butoxy)-2-oxoethyl)zinc(II) chloride, CAS #321745-86-2) under metal-catalyzed conditions (Pd(dba)₂ (CAS #32005-36-0), Q-phos (CAS #312959-24-3)) in THF to provide the corresponding t-butyl ester after purification. S-2 can then be transformed to provide S-3 by the same two-step sequence shown in Scheme 1.

Alternatively, compounds such as S-9, wherein S_{b} is Br in S-3, can be prepared by the general method outlined in Scheme 4.

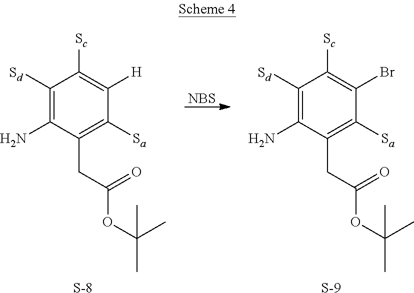

Compound S-8, wherein S_{b} is H in S-3, (when, for example Sₐ, S_{c}, S_{d} are H, CAS #98911-34-3), can be brominated with N-bromosuccinimide (CAS #128-08-5) at low temperature (−30° C.) in a solvent such as dimethylformamide (DMF).

Compounds such as S-12, wherein R⁴ is selected from C₁-C₄-alkyl or alkenyl can be prepared by the general method outlined in Scheme 5.

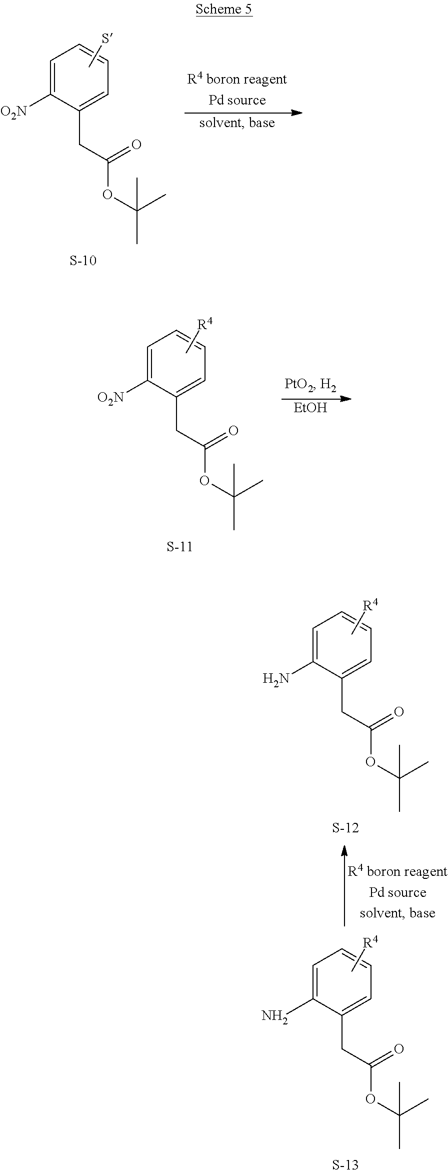

S-10 (for example when S′ is Br and Sₐ in S-2, S_{b}, S_{c}, S_{d} are H, CAS #37777-74-5) or S-13 can be coupled with a boron-containing reagent (e.g. potassium methyltrifluoroborate CAS #13862-28-7) under the catalysis of a Pd source (e.g. PdCl₂(dppf).CH₂Cl₂ adduct, CAS #95464-05-4), in the presence of a base (e.g. Cs₂CO₃) in a suitable solvent system (e.g. toluene/water) to provide compound S-12 (an additional step to reduce the nitro group is required in the case of S-11).

Compounds such as S-16, wherein S_{a-d} is independently selected from halo, alkyl or alkoxyl can be prepared by the general method outlined in Scheme 6.

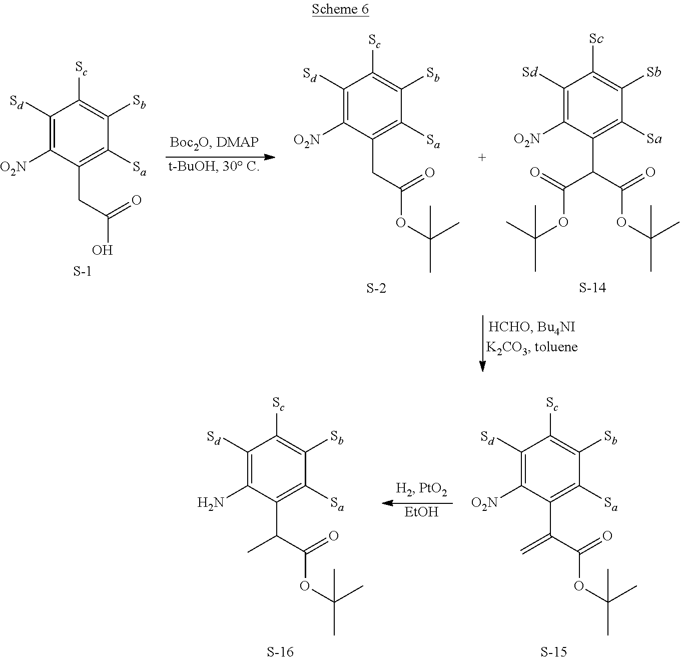

The acid group in S-1 (especially when Sₐ, S_{b}, S_{c}, S_{d} are H, CAS #3740-52-1), can be protected as the corresponding tert-butyl ester (S-2) with treatment of Boc-anhydride and DMAP in t-butanol at a temperature slightly above room temperature such as 30° C. A minor product S-14 is also generated under these conditions. The mixture of S-2 and S-14 can then react with formaldehyde in the presence of tetrabutylammonium iodide (CAS #311-28-4) and potassium carbonate in a solvent such as toluene at 50° C. for 3 days to provide compound S-15. The olefin and nitro groups within S-15 can be then reduced under hydrogen atmosphere with a catalyst such as platinum oxide in a solvent such as ethanol at room temperature to afford S-16.

Compounds such as Q-2, wherein Q_{a-d} is independently selected from halo, C₁-C₄alkyl or C₁-C₄alkoxy and hydroxyC₁-C₄alkyl, can be prepared by the general method outlined in Scheme 7.

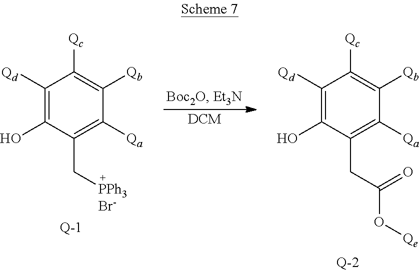

Q-1 (especially when Qₐ, Q_{b}, Q_{c}, Q_{d} are H, CAS #70340-04-4) can react with Boc-anhydride in the presense of triethylamine with dichloromethane as the solvent at 40° C. for 4 days to provide the corresponding t-butyl ester Q-2 (Qₑ is tBu). When Qₐ, Q_{b}, Q_{c}, Q_{d} represent 0, 1, or 2 substituents independently selected from halogen, cyano, C₁-C₄alkyl, C₁-C₄alkoxy, C₃-C₆cycloalkyl, C₃-C₆cycloalkylC₁-C₄-alkyl, C(O)NH₂, NHC(O)C₁-C₄alkyl, CH₂NHC(O)C₁-C₄alkyl, and hydroxyC₁-C₄alkyl they are usually accessed via cross coupling reaction (Pd) especially when one or more Qₐ, Q_{b}, Q_{c}, Q_{d} are Br or CI.

Alternatively, compounds such as Q-2, wherein Q_{a-d} is independently selected from halo, C₁-C₄alkyl or C₁-C₄alkoxy and hydroxyC₁-C₄alkyl, can be prepared by another general method outlined in Scheme 8.

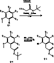

The bromo group of Q-3 (especially when Qₐ, Q_{b}, Q_{c} are H, Q_{d} is F, CAS #845829-94-9) can be coupled with a zinc reagent ((2-(tert-butoxy)-2-oxoethyl)zinc(II) chloride, CAS #321745-86-2) under metal-catalyzed conditions (Pd(dba)₂ (CAS #32005-36-0), Q-phos (CAS #312959-24-3)) in THF to provide the corresponding t-butyl ester after purification. Both the methyl and t-butyl groups of Q-4 can then be removed with treatment of boron tribromide (CAS #10294-33-4) in dichloromethane. The resulting mixture can then react with methanol to provide the corresponding methyl ester, where Qₑ is CH₃ in Q-2.

Compounds such as P-3 can be prepared by the general method outlined in Scheme 9 in which R¹ is hydrogen, phenyl, C₃-C₆cycloalkyl, amido, halo C₁-C₄alkyl or C₁-C₄alkyl optionally substituted by hydroxy, C₃-C₆cycloalkyl, C₁-C₄alkoxy or cyano;

R^{1a} is hydrogen or C₁-C₄alkyl, or CR¹R^{1a} taken in combination form a carbonyl (C═O), imine (C═NH) or a 3-6 member cycloalkyl;

R^{1a} is absent and CR¹ and R¹¹, taken in combination, form a saturated, unsaturated or aromatic 4, 5 or 6 member azacycle;

T is CR² or N;

U is CR¹⁴ or N;

V is CR¹² or N;

W is CR¹³ or N, wherein 0, 1, or 2 of T, U, V and W are N; or

V is N, W is S, T is absent and U is CR¹⁴;

R² and R¹⁴ are independently selected from the group consisting of hydrogen and halogen;

R¹² is hydrogen, halogen, hydroxy, C₁-C₄alkyl or C₁-C₄alkoxy;

R¹³ is hydrogen; or

R¹⁴ is hydrogen or halogen; and

P′ is Cl, Br or I.

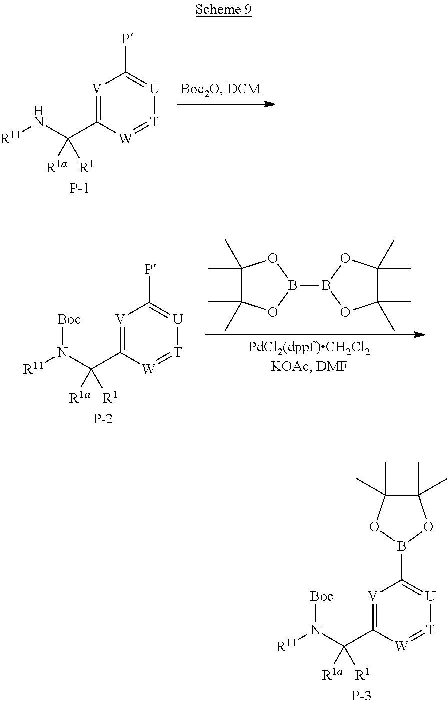

The amino group of P-1 (for example when T, U, V, W are CH, P′ is Br, R¹, R^{1a}, R¹¹ are H, CAS #10269-01-9 or for example when T, U, V, W=CH, P′ is Br, R¹=CH₂OH, R^{1a}, R¹¹ are H, CAS #209963-05-3) can react with Boc-anhydride in dichloromethane with or without a base at room temperature to provide the N-Boc-protected P-2. The aryl halide of P-2 can then be converted to the corresponding pinacol borate P-3 under the typical Miyaura borylation conditions such as (bis(pinacolato)diboron (CAS #73183-34-3), potassium acetate, PdCl₂(dppf).CH₂Cl₂ adduct (CAS #95464-05-4), 110° C., 5 hr).

Compounds such as P-7 can be prepared by the general method outlined in Scheme 10.

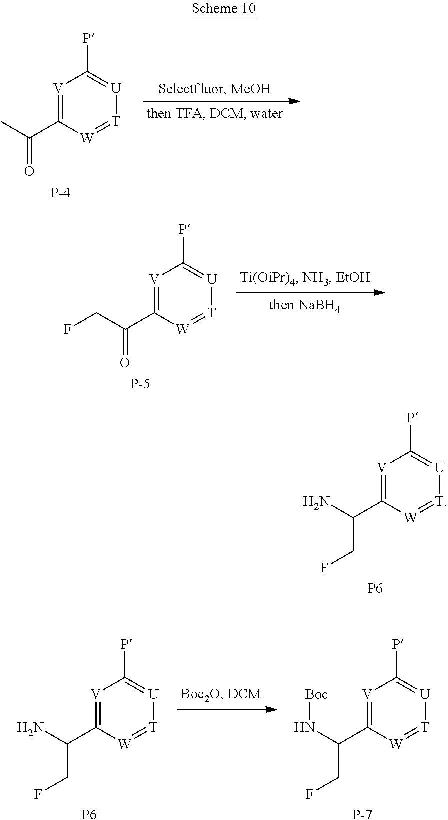

The methyl ketone of P-4 (especially when U, V, W are CH, P′ is Br, CAS #2142-63-4) can be fluorinated with Selectfluor (CAS #140681-55-6) with concurrent partial conversion of the ketone to the corresponding dimethyl ketal. The resulting mixture can then be treated with TFA in a mixture of dichloromethane and water at 40° C. overnight to provide the mono-fluorinated product P-5. Reductive amination of P-5 by a two-step procedure (Ti(OiPr)₄, NH₃, EtOH; then NaBH₄) can produce the corresponding amine, which is then protected with Boc using the typical conditions (Boc-anhydride, dichloromethane) to provide P-7.

Compounds such as P-1, when not commercially available, can be prepared by the general method outlined in Scheme 11.

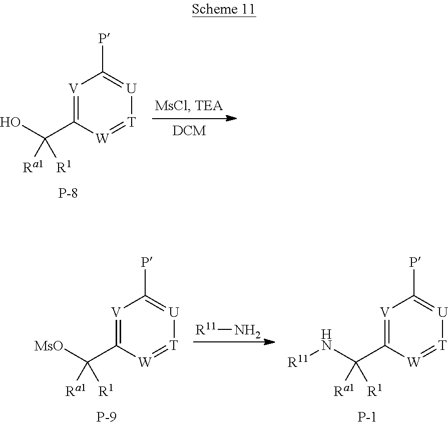

The benzyl alcohol of P-8 (especially when T, U, W are CH, V is CCl, P′ is Br, R¹, R^{1a} are H, CAS #1261524-75-7) can react with MsCl in the presence of triethylamine in dichloromethane at 0° C. to generate the corresponding mesylate P-9, which can then react with an amine (especially when R¹¹ is H) to provide P-1.

Compounds such as P-1d, when not commercially available, can be prepared by the general method outlined in Scheme 12.

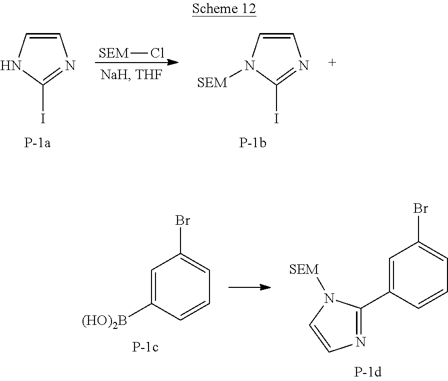 [SMILES: Ic1ncc[nH]1 ; C[Si](C)(C)CCOCCl ; C[Si](C)(C)CCOCn1ccnc1I ; OB(O)c1cccc(Br)c1 ; C[Si](C)(C)CCOCn1ccnc1-c1cccc(Br)c1]

2-Iodo-1H-imidazole (P-1a, CAS #3034-62-6) can react with SEM-Cl in the presence of sodium hydride in THF to provide compound P-1b, which can then couple with (3-bromophenyl)boronic acid (P-1c, CAS #89598-96-9) under typical Suzuki coupling conditions (such as (PdCl₂(dppf).CH₂Cl₂, K₃PO₄, DMF, 110° C., 1 hr) to provide P-1d.

Intermediates such as P-11 can be prepared by the general method outlined in Scheme 13, wherein:

B is CR³ or N;

X is CR⁶ or N;

Y is CR⁵ or N;

Z is CR⁷ or N, wherein 0 or 1 of B, X, Y and Z are nitrogen; or

X is N, B is CR³ and one of Y or Z is S or N(H) and other of Y or Z is absent;

R² and R¹⁴ are independently selected from the group consisting of hydrogen and halogen;

R³ is hydrogen, halogen, hydroxy, cyano, amino, NHR⁸, N(R⁸)₂, —C(O)NHR⁸, OR⁹, C₁-C₄alkyl, haloC₁-C₄alkyl, C₃-C₆cycloalkyl, phenyl, heterocycloalkyl having 4 to 7 ring atoms and 1, 2, or 3 ring heteroatoms independently selected from N, O and S, and heteroaryl having 5, 6, 9 or 10 ring atoms and 1, 2, or 3 ring heteroatoms independently selected from N, O and S, wherein each heterocycloalkyl, heteroaryl, phenyl is optionally substituted with 0, 1, 2, or 3 substituents independently selected from C₁-C₄alkyl, C₁-C₄alkoxy, halogen, or C₃-C₆cycloalkyl, wherein each heterocycloalkyl or heteroaryl is optionally further substituted by 0 or 1 phenyl groups and wherein each alkyl, haloalkyl and cycloalkyl group is optionally substituted with 0, 1, or 2 substituents independently selected from the group consisting of hydroxy, C₃-C₆cycloalkyl, amino, NHR⁸, N(R⁸)₂, OR⁹ and heterocycloalkyl having 4 to 7 ring atoms and 1, 2, or 3 ring heteroatoms independently selected from N, O and S;
R⁴ represents 0, 1, or 2 substituents independently selected from halogen, C₁-C₄alkyl, C₁-C₄alkoxy, and hydroxyC₁-C₄alkyl;

R⁵ is hydrogen, halogen, C₁-C₄alkyl or C₁-C₄alkoxy;

R⁶ is hydrogen, halogen, C₁-C₄alkyl or C₁-C₄alkoxy;

R⁷ is hydrogen, halogen, C₁-C₄alkyl, C₁-C₄alkoxy, haloC₁-C₄alkyl or haloC₁-C₄alkoxy, or

R⁷ is phenyl or a 5 or 6 member heteroaryl having 1, 2, or 3 ring heteroatoms selected from N, O or S; each of which is optionally substituted by 0, 1, or 2 substituents selected from C₁-C₄alkyl, aminoC₁-C₄alkylalkyl, hydroxy₁-C₄alkylalkyl, halogen, C₁-C₄alkoxy, hydroxy, amino or mono- or di-C₁-C₄alkylamino; or

R³ and either R⁵ or R⁷ taken in combination form a —O(CH₂)ₙO— group wherein n is 1 or 2; or

R³ and R⁷ taken in combination with the atoms to which they are attached form a 5 or 6 member aromatic heterocycle having 1 or 2 ring heteroatoms selected from N, O or S and which is optionally substituted with C₁-C₄alkyl, C(O)C₁-C₄alkyl, C(O)NH2, C(O)NHC₁-C₄alkyl, C(O)N(C₁-C₄alkyl)₂, S(O)₂C₁-C₄alkyl, S(O)₂C₃-C₆cycloalkyl, optionally substituted S(O)₂phenyl, where the phenyl is optionally substituted with 0, 1, or 2 C₁-C₄alkyl, or C₁-C₄alkoxy or C₁-C₄alkoxy;

P″ is Cl, Br, or I.

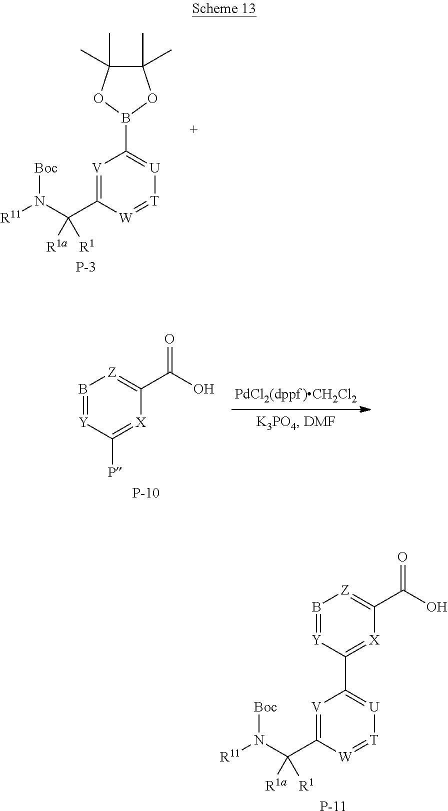

P-3 and P-10 can react under typical Suzuki coupling conditions such as (PdCl₂(dppf).CH₂Cl₂, K₃PO₄, DMF, 110° C., 1 hr) to provide compound P-11. Intermediates such as P-13a (where P′″ is OMe or O^{t}Bu) can be prepared by the general method outlined in Scheme 14.

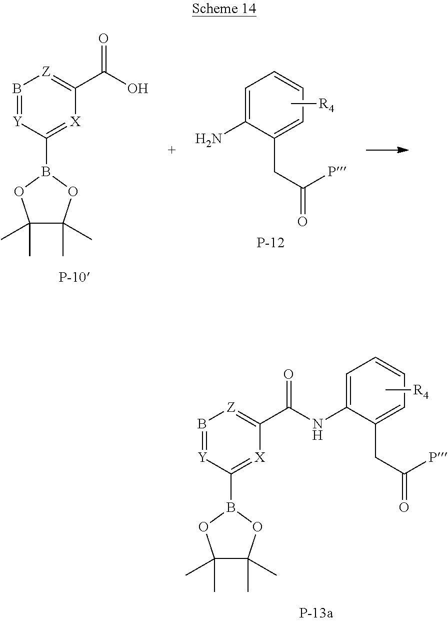

P-10′ and P-12 can be coupled in the presence of HATU (CAS #148893-10-1) and diisopropylethylamine in DMF at room temperature to provide the corresponding amide P-13a. Intermediates such as P-13 and P-10 (when not commercially available) can be prepared by the general method outlined in Scheme 15.

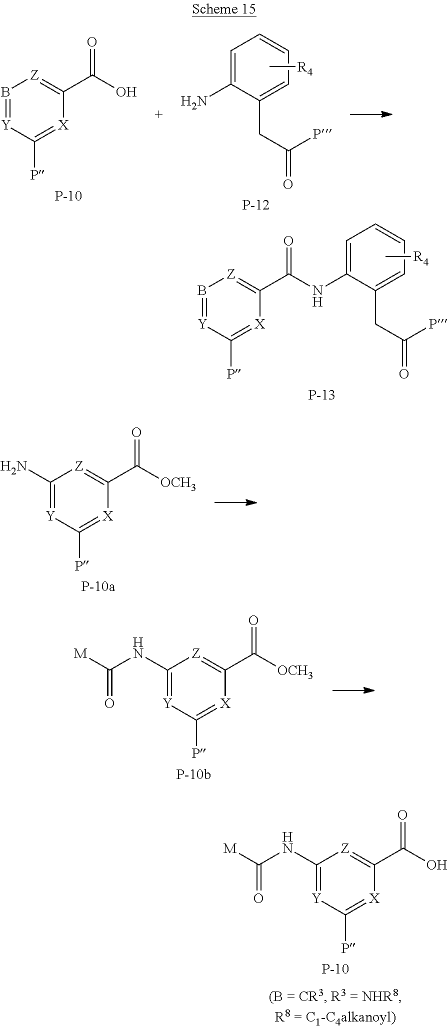

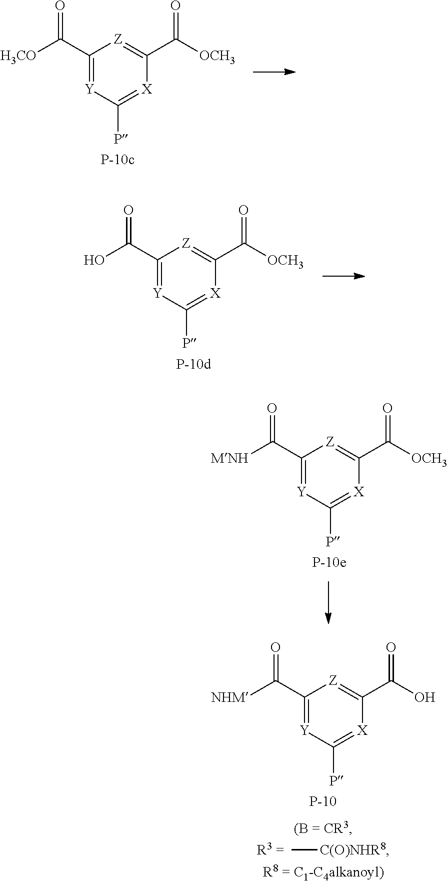

The acid group of P-10 and the amine group of P-12 (especially when P′″ is OCH₃) can be coupled in the presence of HATU (CAS #148893-10-1) and diisopropylethylamine in DMF at room temperature to provide the corresponding amide P-13. In case where P-10 is not commercially available (especially when B is CR₃, R₃ is NHR₈, R₈ is C₁-C₄alkanoyl), it can be synthesized from P-10a. P-10a can react with appropriate alkyl acid under typical amide coupling conditions (such as HATU, diisopropylethylamine, DMF, room temperature, overnight) to provide P-10b, which upon hydrolysis under basic conditions (such LiOH, THF, water, room temperature) can give P-10. Alternatively, in case where P-10 is not commercially available (especially when B is CR₃, R₃ is —C(O)NHR₈, R₈ is C₁-C₄alkyl), it can be synthesized from P-10c. P-10c can be hydrolyzed under basic conditions (such as LiOH, THF, water) to provide P-10d, which can then react with appropriate alkylamine via a 2-step procedure (oxalyl chloride, DMF; then M′NH₂) to provide P-10e. The methyl ester in P-10e can then be hydrolyzed under basic conditions (such as LiOH, THF, water) to give P-10 wherein B is CR₃, R₃ is —C(O)NHR₈, R₈ is C₁-C₄alkyl.

Intermediates such as P-16 and P-17, where A is —CHR¹⁰O—, R¹⁰ is as described in the embodiment one, can be prepared by the general method outlined in Scheme 16.

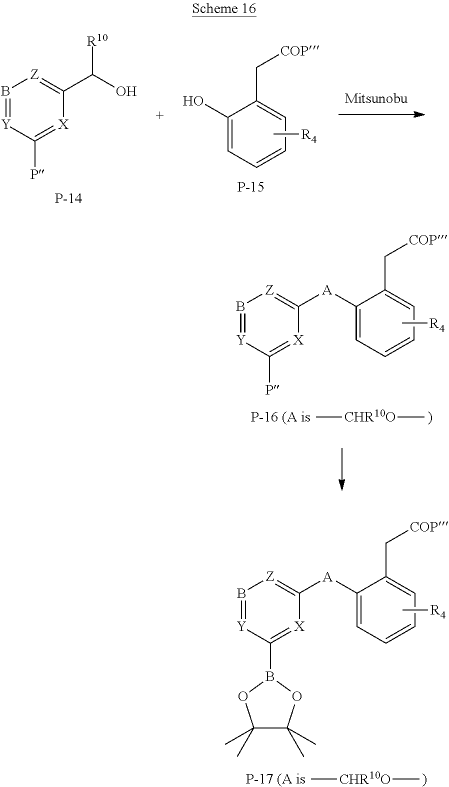

Compound P-14 (when for example B, X, Y, Z are CH, P″=Br and R¹⁰=H, CAS #15852-73-0) and P-15 can react under typical Mitsunobu reaction conditions (such as PPh₃, DIAD, THF, 0-23° C., overnight) to provide P-16, where A is —CHR¹⁰O—. P-16 can then be transformed to the corresponding pinacol borate under typical Miyaura coupling conditions (such as bis(pinacolato)diboron (CAS #73183-34-3), potassium acetate, PdCl₂(dppf).CH₂Cl₂ adduct (CAS #95464-05-4), 110° C., 5 hr) to provide P-17 where A is —CHR¹⁰O—.

Intermediates such as P-16 where A is —OCH₂— can be prepared by the general method outlined in Scheme 17.

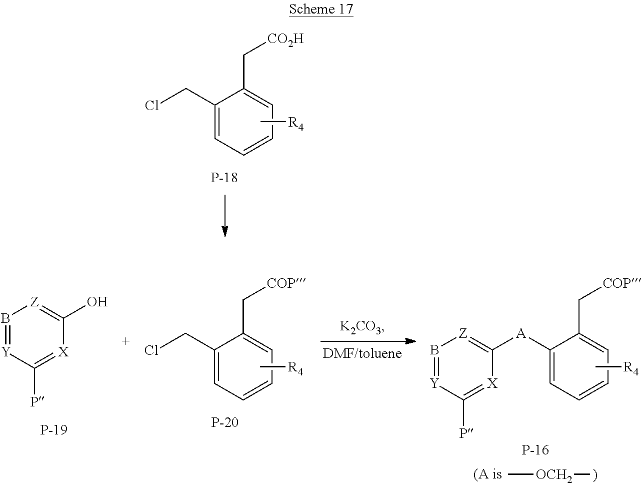

Acid P-18 (for example when R⁴=H, CAS #95335-46-9) can be converted to the corresponding ester P-20 (where P′″ is OMe) via a 2-step process (oxalyl chloride, DMF, DCM; then MeOH, DIPEA). Ester P-20 and P-19 (for example when X=N and B, Y, Z=CH and P″=Cl, CAS #73018-09-4) can then react in the presence of a base (such as potassium carbonate) in a solvent system (such as DMF/toluene) to provide P-16, where A is —OCH₂—.

Intermediates such as P-13, where B is CR³, R³ is OR⁹, can be prepared by the general method outlined in Scheme 18.

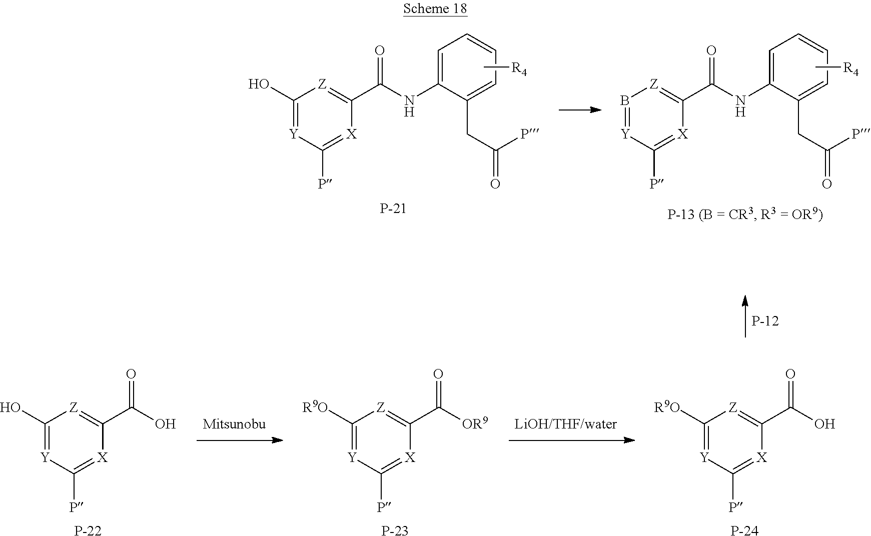

Compound P-21 (for example when X, Y, Z are CH, R⁴ is H, P′″ is Ot-butyl, P″ is Br, which in this example is derived from coupling 3-bromo-5-hydroxybenzoic acid, CAS #140472-69-1, as in Scheme 15) can react with an alcohol such as pyrimidin-2yl-methanol (CAS #42839-09-8) under typical Mitsunobu reaction conditions (such as PPh₃, DIAD, THF, 0-23° C., overnight) to provide P-13. Alternatively, P-13 can also be synthesized starting from the acid P-22. Acid P-22 (for example when X, Y, Z are CH, R⁴ is H, R is t-butyl, P″ is Br, CAS #140472-69-1) can react with an alcohol R⁹OH under typical Mitsunobu reaction conditions (such as PPh₃, DIAD, THF, 0-23° C., overnight) to provide P-23. The ester group in P-23 can be hydrolyzed under basic conditions (such as LiOH, THF, H₂O, room temperature) to provide acid P-24, which can then couple with P-12 under typical amide coupling conditions (such as HATU, diisopropylethylamine, DMF, room temperature, overnight) to provide the corresponding amide P-13.

Alternatively, intermediates such as P-16 (especially when B is CR³, R³ is OR⁹) can be prepared by the general method outlined in Scheme 19.

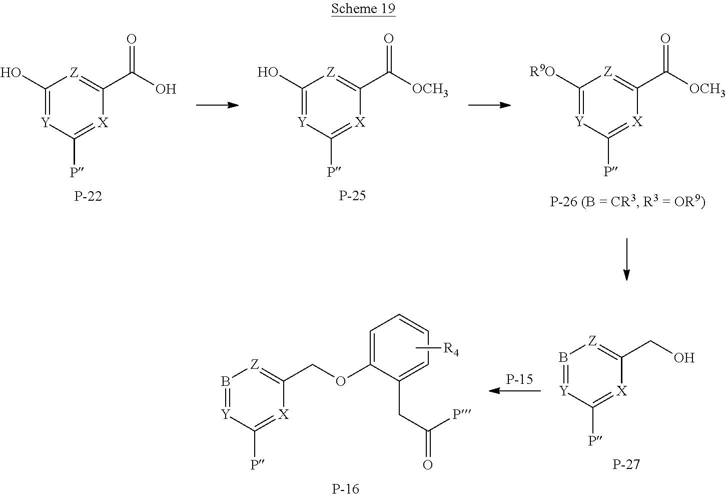

The acid group of compound P-22 (especially when X, Y, Z are CH, P″ is Br, CAS #140472-69-1) can be esterified in a mixture of aqueous HCl and methanol at room temperature to form compound P-25. P-25 and an alcohol R⁹OH can then react under typical Mitsunobu reaction conditions (such as PPh₃, DIAD, THF, 0-23° C., overnight) to provide P-26. Alternatively, (especially when R⁹ is CF₃CH₂—), P-25 can react with 2,2,2-trifluoroethyl trifluoromethanesulfonate in the presence of potassium carbonate in DMF to provide the corresponding P-26. P-26 can then be reduced with a reducing agent (such as LiBH₄) in a solvent (such as THF) at an elevated temperature (such as 50° C.) to provide the corresponding alcohol P-27, which is then reacted with P-15 again under typical Mitsunobu reaction conditions (such as PPh₃, DIAD, THF, 0-23° C., overnight) to provide P-16.

Intermediates such as P-16, where B is CR³, R³ is NHR⁸, can be prepared by the general method outlined in Scheme 20.

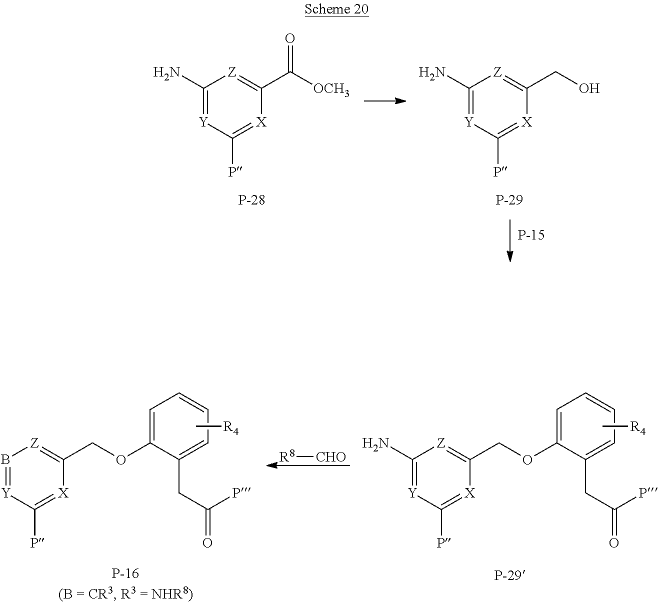

P-28 (for example when X, Y=CH and Z=CF, P″=Br, CAS #1339049-19-2) can then be reduced with a reducing agent (such as LiBH₄) in a solvent (such as THF) at an elevated temperature (such as 50° C.) to provide the corresponding alcohol P-29. P-29 and P-15 can then react under typical Mitsunobu reaction conditions (such as PPh₃, DIAD, THF, 0-23° C., overnight) to provide P-29′, which is then reacted with an aldehyde (R⁸—CHO) under typical reductive amination conditions (such as NaBH(OAc)₃, dichloroethane, room temperature overnight) to provide P-16, where B is CR₃ and R₃ is NHR₈.

Intermediates such as P-30 or the corresponding salt of P-30s, wherein X is N, B is CR³ and one of Y or Z is N(H) and other of Y or Z is absent, can be prepared by the general method outlined in Scheme 21.

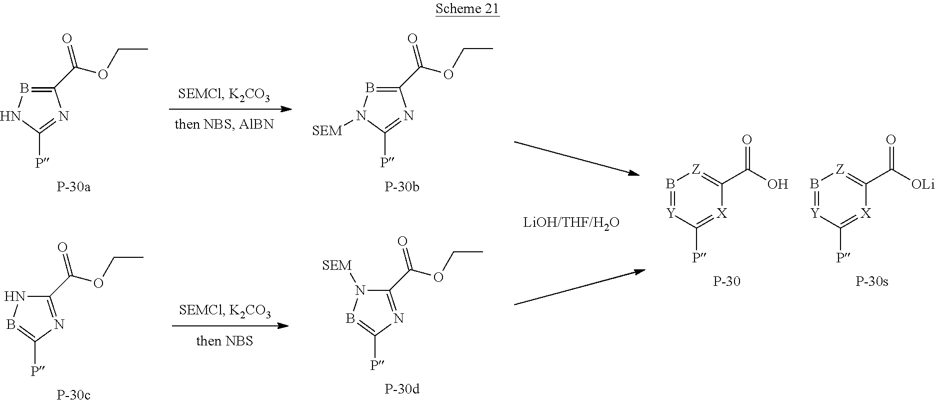

P-30a/P-30c (especially when B=CH and P″=H respectively CAS #23785-21-9 and CAS #33543-78-1) can react with SEM-CI in the presence of K₂CO₃ in DMF and then with NBS and AIBN in CCl₄ at high temperatures such as 60° C. (for P-30a) or with NBS in DMF/DCM (for P-30c) at rt to provide compound P30b/P-30d, respectively. The ester group of P-30c/P-30d can be hydrolyzed under basic conditions (such as LiOH, THF, H₂O) to provide compound P-30 or P-30s, where X is N, B is CR³ and one of Y or Z is N(H) and other of Y or Z is absent.

Intermediates such as P-33, can be prepared by the general method outlined in Scheme 22.

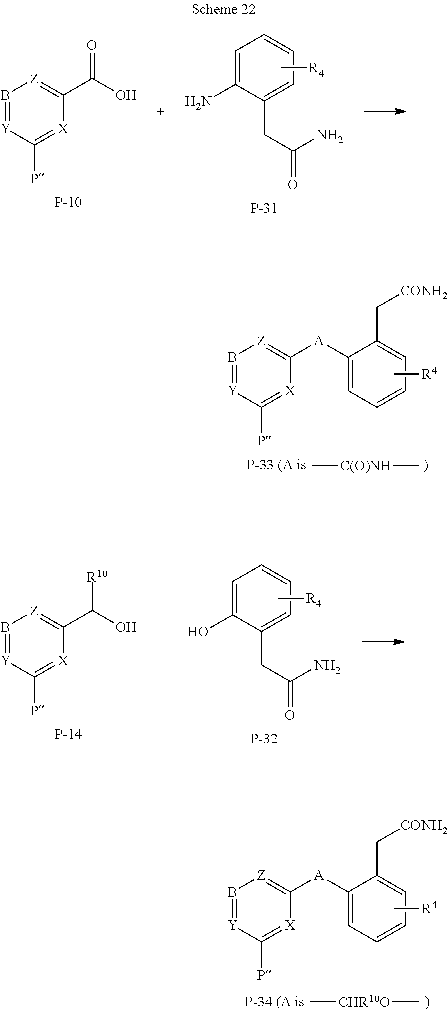

The acid group of P-10 (especially when B, X, Y, Z are CH, P″ is Br, CAS #585-76-2) and the amine group of P-31 (especially when R₄, Q″ are H, CAS #4103-60-0) can be coupled under typical amide coupling conditions (such as HATU, diisopropylethylamine, DMF, room temperature, overnight) to provide the corresponding amide P-33 (A is —C(O)NH—). Compounds P-14 and P-32 can react under the typical Mitsunobu conditions to provide P-34.

Intermediates such as P-38, where A is —NHCH₂—, can be prepared by the general method outlined in Scheme 23.

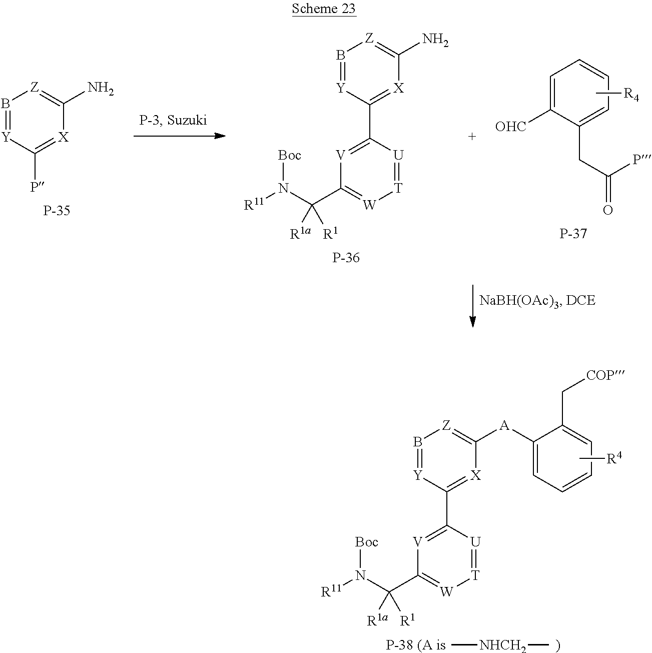

Compound P-35 (especially when B, X, Y, Z are CH, P″ is Br, CAS #591-19-5) can react with P-3 under typical Suzuki coupling conditions (such as PdCl₂(dppf).CH₂Cl₂, K₃PO₄, DMF, 110° C., 1 hr) to provide P-36, which can then couple with P-37 under typical reductive amination conditions (such as NaBH(OAc)₃, DCE, room temperature) to provide compound P-38, where A is —NHCH₂—.

Intermediates such as P-16, wherein A is —S(O)₂N(H)—, can be prepared by the general method outlined in Scheme 24.

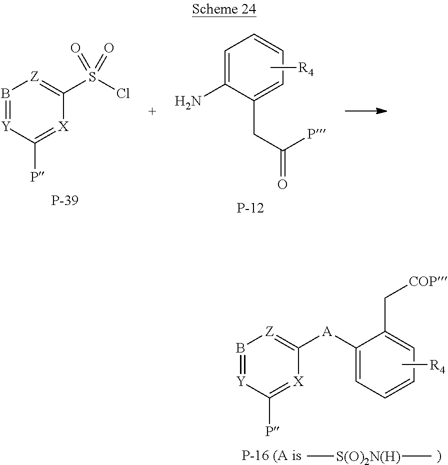

Compound P-39 (especially when B, X, Y, Z are CH, P″ is Br, CAS #2905-24-0) can react with P-12 in the presence of a base (such as pyridine) to provide compound P-16, where A is —S(O)₂N(H)—.

Intermediates such as P-38, wherein A is —CH₂CH₂— or —C≡C—, can be prepared by the general method outlined in Scheme 25.

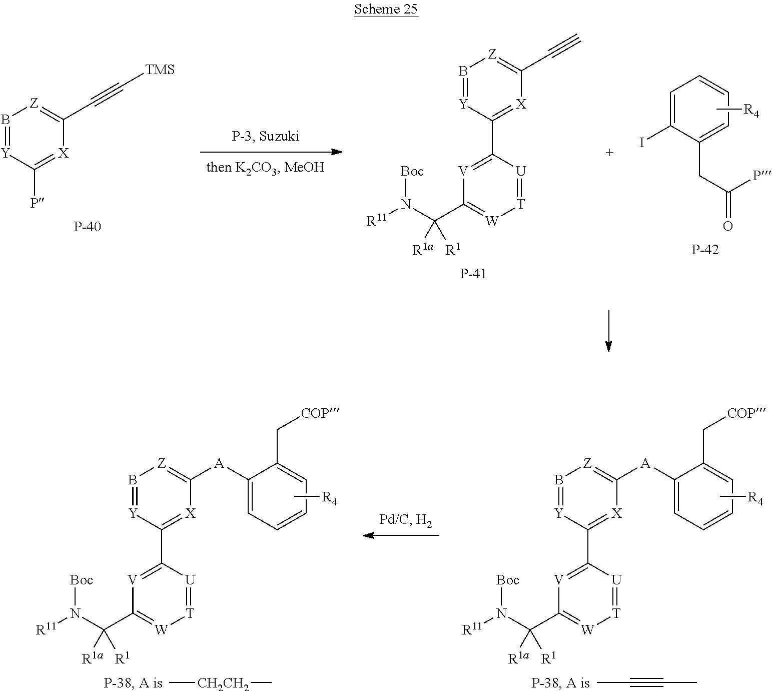

Compound P-40 (especially when B, X, Y, Z are CH, P″ is Br, CAS #3989-13-7) can react with P-3 under typical Suzuki coupling conditions (such as PdCl₂(dppf).CH₂Cl₂, K₃PO₄, DMF, 110° C., 1 hr). The resulting coupling product can then be treated with potassium carbonate in MeOH to remove the TMS group to provide P-41. Compound P-41 can couple with P-42 (especially when R⁴ is H, R is CH₃, CAS #66370-75-0) under typical Sonogashira coupling conditions (such as Cul, Pd(PPh₃)₄, toluene, room temperature, overnight) to provide P-38 where A is —C≡C—. P-38, where A is —C≡C—, can then further react with hydrogen gas in the presence of a catalyst such as Pd/C to provide P-38, where A is —CH₂CH₂—.

Compounds such as P-45 can be prepared by the general methods outlined in Scheme 26

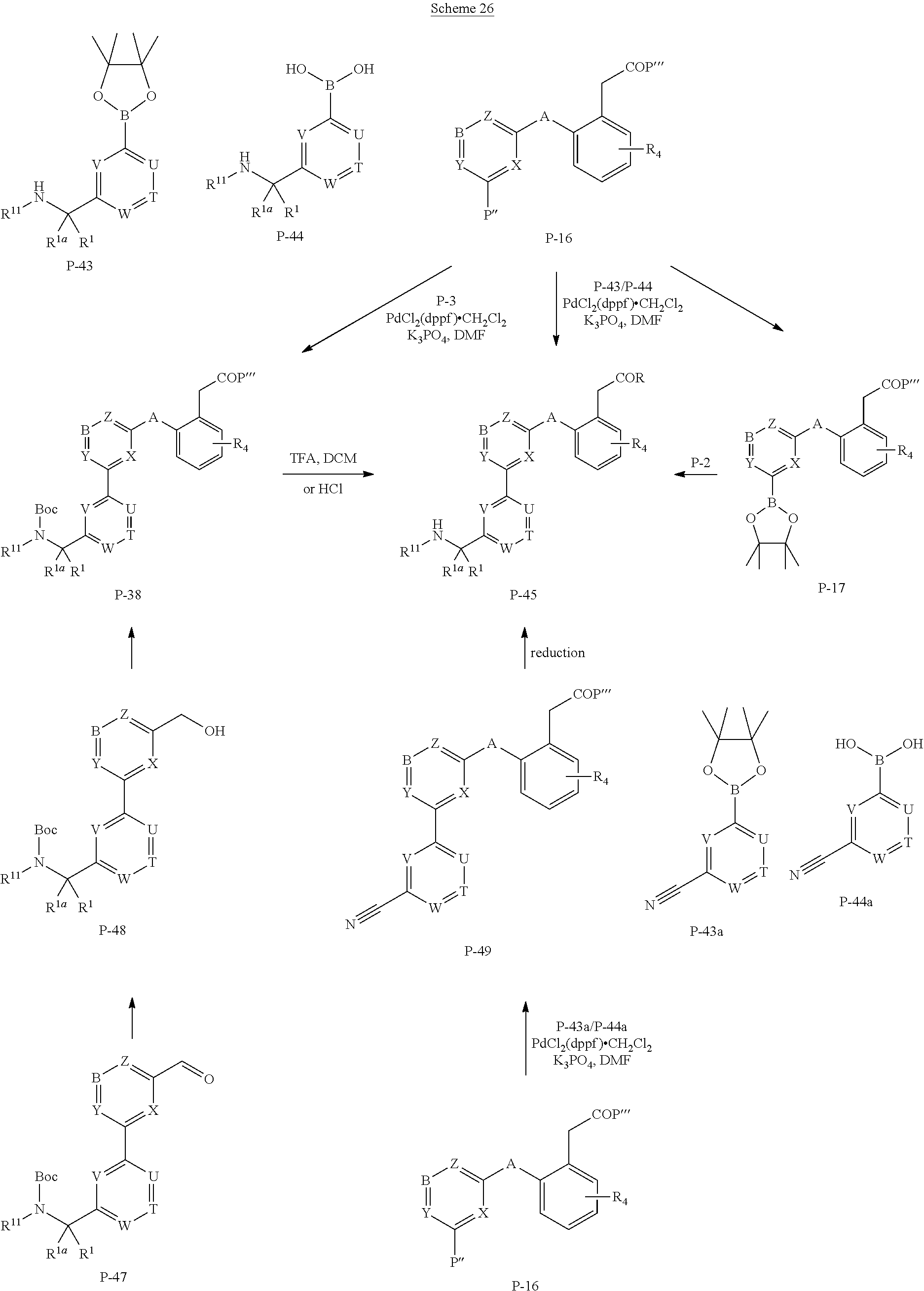

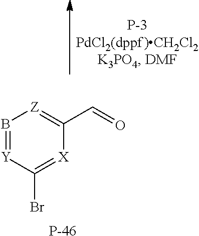

P-16 can then couple with P-43 or P-44 under the typical Suzuki coupling conditions (such as PdCl₂(dppf).CH₂Cl₂, K₃PO₄, DMF, 110° C., 1 hr) to provide compound P-45. When P′″=O^{t}Bu, the compound can be deprotected with TFA/DCM at temperatures ranging from 0° C. to rt or HCl (4 M in dioxane) at temperatures ranging from 0° C. to rt; when P′″=C₁-C₄alkoxy, the compound can be deprotected with LiOH in mixture of MeOH/THF/H₂O or CH₃CN at temperatures ranging from 0° C. to 60° C. to provide compound P-45 (where R=OH); or P-16 can react with P-3 under similar conditions to provide P-38. Alternatively, P-38 can also be synthesized from P-46 via a 3-step procedure (Suzuki coupling, then reduction of aldehyde, Mitsunobu reaction). P-38, when P′″=O^{t}Bu or NH₂ or OH, can be deprotected with TFA/DCM at temperatures ranging from 0° C. to rt or HCl (4 M in dioxane) at temperatures ranging from 0° C. to rt to give P-45; when P′″=C₁-C₄alkoxy, P-38 is deprotected with TFA/DCM at temperatures ranging from 0° C. to rt or HCl (4 M in dioxane) at temperatures ranging from 0° C. to rt to give P-45 (in which R=C₁-C₄alkoxy), and then LiOH in mixture of MeOH/THF/H₂O or CH₃CN at temperatures ranging from 0° C. to 60° C. (or vice versa) to provide compound P-45 (in which R=OH). Alternatively, P-16 can also be converted to the corresponding pinacol borate P-17 under typical Miyaura coupling conditions (such as bis(pinacolato)diboron (CAS #73183-34-3), potassium acetate, PdCl₂(dppf).CH₂Cl₂ adduct (CAS #95464-05-4), 110° C., 5 hr). P-17 can then react with P-2 under typical Suzuki coupling conditions (such as PdCl₂(dppf).CH₂Cl₂, K₃PO₄, DMF, 110° C., 1 hr) to provide P-45 (deprotection strategy same as for P-38). Alternatively, P-16 can react with P-43a or P-44a under typical Suzuki coupling conditions to provide P-49, which can react under reducing conditions (such as NiCl₂, NaBH₄, EtOH; or H₂, Pd/C, MeOH) to provide P-45 (deprotection strategy same as for product of reaction of P-16 with P-43/P-44).

Alternatively, compounds such as P-45 and P-45a (especially Z is CR⁷, R⁷ is aryl or heteroaryl), can be prepared by the general methods outlined in Scheme 27.

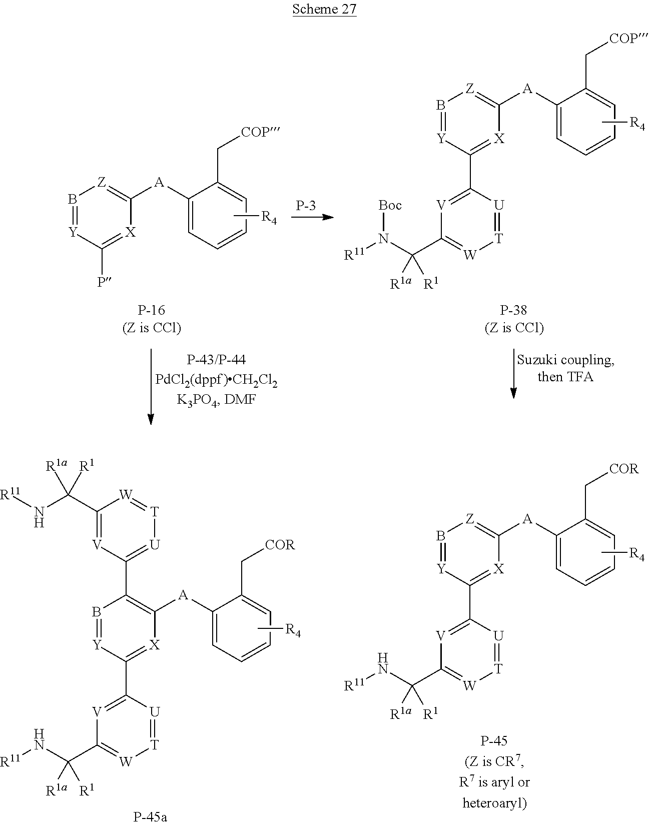

P-16 can couple with P-3 under typical Suzuki coupling conditions to provide P-38, where Z is CCl. And P-38 can then couple with a different aryl/heteroaryl boronic acid or ester under typical Suzuki coupling conditions provide P-45 (deprotection strategy same as for product of reaction of P-16 with P-43/P-44) where Z is CR⁷ and R⁷ is aryl or heteroaryl. Or P-16 can couple with P-43/P-44 under typical Suzuki coupling conditions to provide P-45a (deprotection strategy same as for product of reaction of P-16 with P-43/P-44).

Alternatively, compounds such as P-38 (especially when B is CR³), wherein P-49 Q′ is Cl, Br, can be prepared by the general method outlined in Scheme 28.

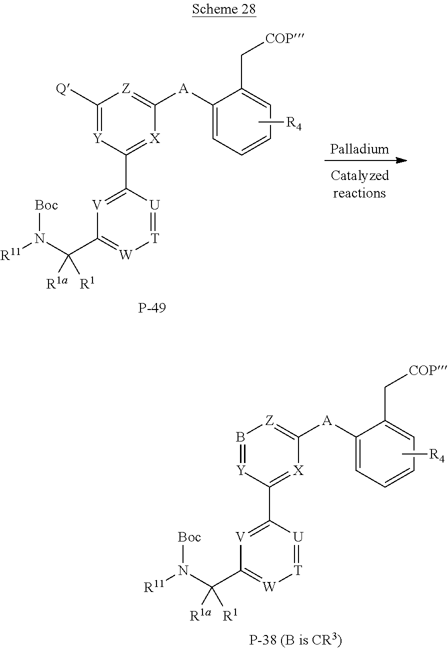

Compound P-49 can undergo typical Pd-catalyzed cross-coupling reaction such as Suzuki reaction and Buchwald-Hartwig amination to provide P-38. For example, when X, Y, Z, U, V, W are CH, R¹, R^{1a}, R⁴ and R¹¹ are H, P′″ is —OtBu, Q′ is Br, T is CF, A is —C(O)NH—, P-49 can couple with isoxazol-4-ylboronic acid in the presence of a palladium catalyst such as X-Phos palladacycle (CAS #1028206-56-5) and a base such as potassium phosphate in a solvent such as a mixture of DMF and water after being heated at 90° C. for 2 hr to provide P-38. Or, when X, Y, Z, U, V, W are CH, R¹, R^{1a}, R⁴ and R¹¹ are H, P′″ is —OtBu, Q′ is Br, T is CF, A is —C(O)NH—, P-49 can couple with an aniline in the presence of a palladium catalyst such as X-Phos palladacycle and a base such as potassium phosphate in a solvent such as CH₃CN after being heated at 90° C. for 2 hr to provide P-38. Or, when X, Y, Z, U, V, W are CH, R¹, R^{1a}, R⁴ and R¹¹ are H, P′″ is —OtBu, Q′ is Br, T is CF, A is —C(O)NH—, P-49 can couple with an alcohol such as (S)-(tetrahydrofuran-2-yl)methanol (CAS #57203-01-7) in the presence of a phosphine ligand such as Rockphos (CAS #1262046-34-3), a palladium source such as allylpalladium chloride dimer (CAS #12012-95-2) and a base such as Cs₂CO₃ in a solvent such as toluene after being heated at 90° C. overnight to provide P-38. P-38 can be deprotected as shown in Scheme 26.

Alternatively, compounds such as P-38 (especially when B is CR³, and R³ is NHR⁸, N(R⁸)₂), wherein R⁸ is independently selected at each occurrence from the group consisting of C₁-C₄alkyl, haloC₁-C₄alkyl, C₃-C₆cycloalkyl, benzyl, C₁-C₄alkanoyl, benzoyl, phenyl, 4 to 6 member heterocycloalkyl, heteroaryl, wherein C₁-C₄alkyl is optionally substituted with C₁-C₄alkoxy, C₃-C₆cycloalkyl, cyano, 4 to 6 member heterocycloalkyl or heteroaryl, wherein phenyl or benzyl are optionally substituted with 0, 1, or 2 substituents selected from C₁-C₄alkyl, CH₂CO₂H, C₃-C₆cycloalkyl or C₁-C₄alkoxy, and wherein each heterocycloalkyl or heteroaryl are optionally substituted by 0, 1, or 2 substituents independently selected from C₁-C₄alkyl, CO₂ C₁-C₄alkyl, C(O)NH C₁-C₄alkyl, C₃-C₆cycloalkyl, or C₁-C₄alkoxy, can be prepared by the general method outlined in Scheme 29.

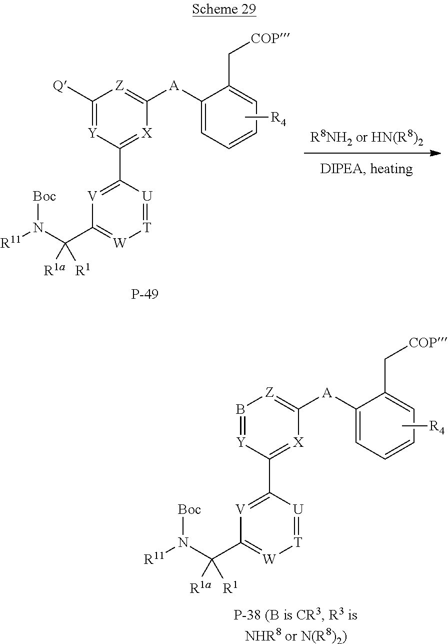

Compound P-49 (especially when X is N, Y, U, V, W are CH, R¹, R^{1a}, R⁴ and R¹¹ are H, Z is CH, P′″ are —OtBu, Q′ is Br, T is CF) can react with an amine such as methylamine in a presence of a base such as diisopropylethylamine in a solvent such as DMSO at elevated temperature such as 180° C. for a period of time such as 30 min to provide P-38, wherein R³ is R⁸NH or N(R⁸)₂), which can then be deprotected to provide P-45 as shown in Scheme 26.

Alternatively, compounds such as P-45 (especially when B is CR³, and R³ is NHR⁸, N(R⁸)₂), can be prepared by the general method outlined in Scheme 30.

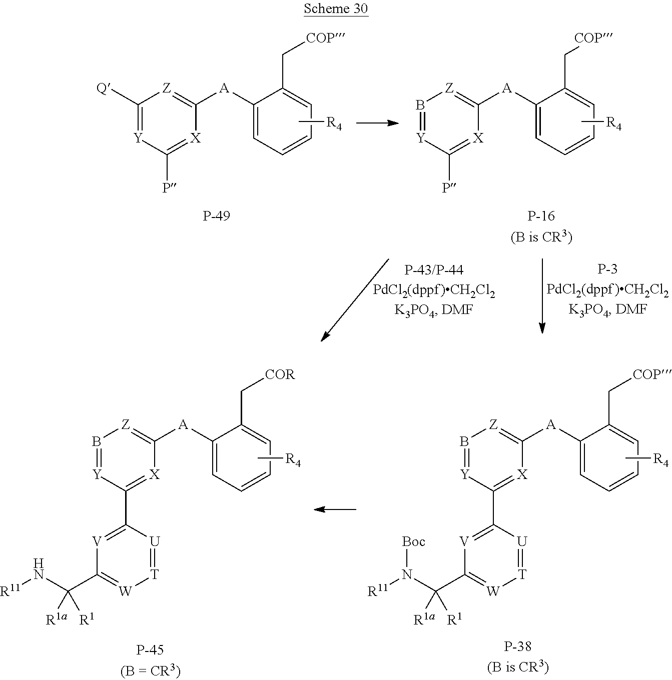

Compound P-49 can undergo typical Pd-catalyzed cross-coupling reaction such as Suzuki reaction and Buchwald-Hartwig amination to provide P-16. In case wherein R³ is NHR⁸, N(R⁸)₂, P-16 can then couple with P-3 under the typical Suzuki coupling conditions (such as PdCl₂(dppf).CH₂Cl₂, K₃PO₄, DMF, 110° C., 1 hr) to provide compound P-38 (which can be transformed to P-45 using the deprotection strategy shown in Scheme 26). Alternatively, P-16 (wherein R³ is NHR⁸, N(R⁸)₂) can couple under typical Suzuki coupling conditions with P-43 or P-44 to directly provide P-45 (deprotection strategy same as for product of reaction of P-16 with P-43/P-44 in Scheme 26).

Compounds such as P-16 especially when B is CR³ and R³ is alkyl, haloalkyl and cycloalkyl group which is optionally substituted with 0, 1, or 2 substituents independently selected from the group consisting of hydroxy, C₃-C₆cycloalkyl, amino, NHR⁸, N(R⁸)₂, OR⁹ and where:

R⁸ is independently selected at each occurrence from the group consisting of C₁-C₄alkyl, haloC₁-C₄alkyl, C₃-C₆cycloalkyl, benzyl, C₁-C₄alkanoyl, benzoyl, phenyl, 4 to 6 member heterocycloalkyl, heteroaryl, wherein C₁-C₄alkyl is optionally substituted with C₁-C₄alkoxy, C₃-C₆cycloalkyl, cyano, 4 to 6 member heterocycloalkyl or heteroaryl, wherein phenyl or benzyl are optionally substituted with 0, 1, or 2 substituents selected from C₁-C₄alkyl, CH₂CO₂H, C₃-C₆cycloalkyl or C₁-C₄alkoxy, and wherein each heterocycloalkyl or heteroaryl are optionally substituted by 0, 1, or 2 substituents independently selected from C₁-C₄alkyl, CO₂ C₁-C₄alkyl, C(O)NH C₁-C₄alkyl, C₃-C₆cycloalkyl, or C₁-C₄alkoxy;

R⁹ is independently selected at each occurrence from the group consisting of C₁-C₄alkyl, haloC₁-C₄alkyl, C₃-C₆cycloalkyl, benzyl, benzoyl, phenyl, 4 to 6 member heterocycloalkyl, heteroaryl, wherein C₁-C₄alkyl is optionally substituted with C₁-C₄alkoxy, C₃-C₆cycloalkyl, cyano, 4 to 6 member heterocycloalkyl or heteroaryl, wherein phenyl or benzyl are optionally substituted with 0, 1, or 2 substituents selected from C₁-C₄alkyl, CH₂CO₂H, C₃-C₆cycloalkyl or C₁-C₄alkoxy, and wherein each heterocycloalkyl or heteroaryl are optionally substituted by 0, 1, or 2 substituents independently selected from C₁-C₄alkyl, CO₂ C₁-C₄alkyl, C(O)NH C₁-C₄alkyl, C₃-C₆cycloalkyl, or C₁-C₄alkoxy;

can be prepared by the general scheme outlines in Scheme 31.

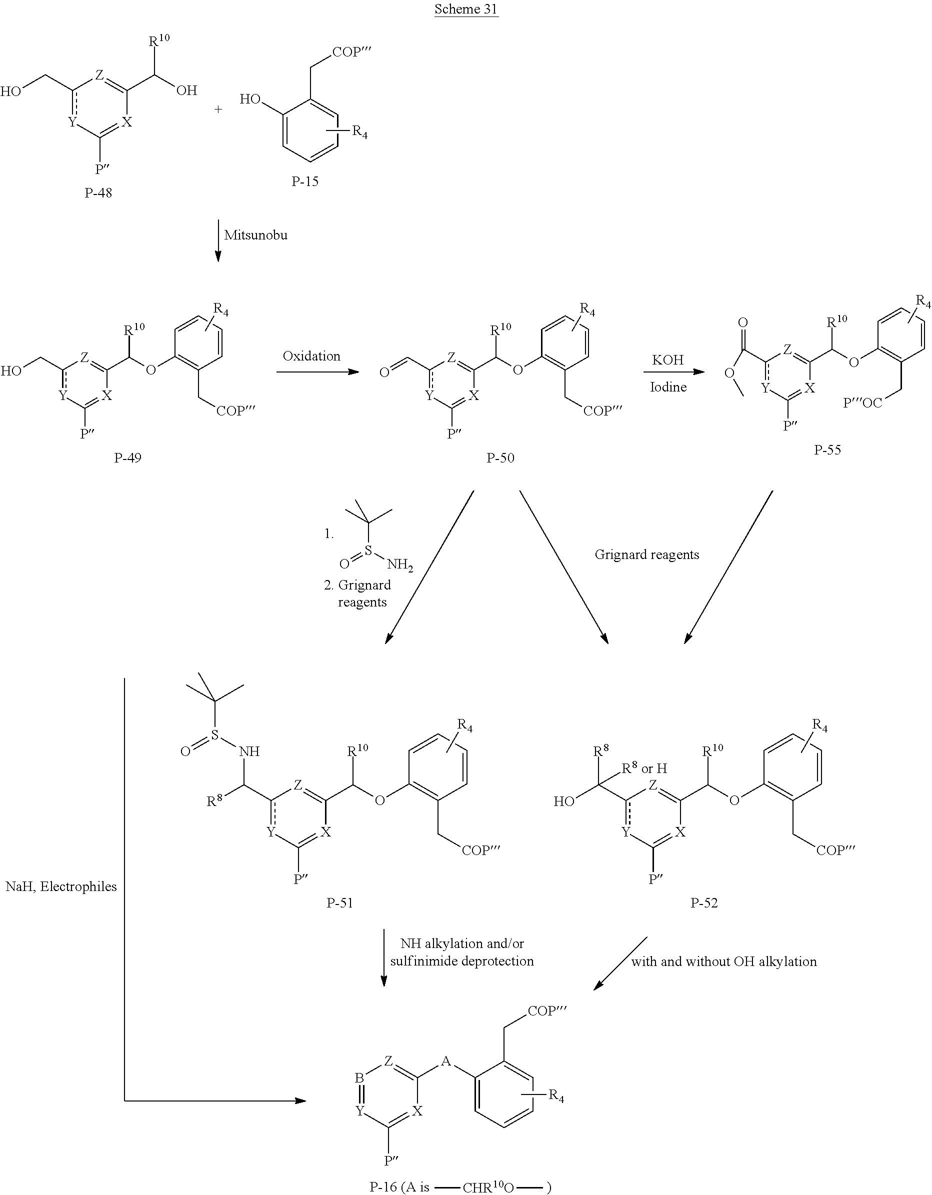

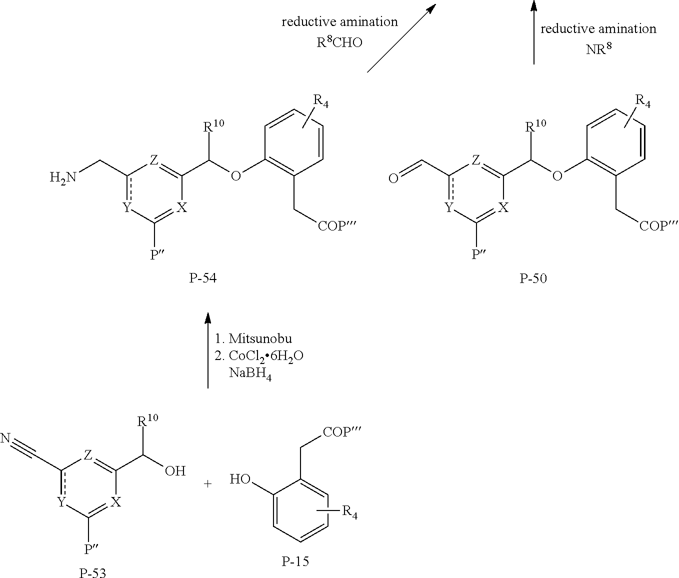

P-48, where for example R³ is —CH₂—OH (especially when X=Y=Z=R¹⁰=H and P″ is Br, CAS #51760-22-6) can react with P-15 under typical Mitsunobu reaction conditions (such as PPh₃, DIAD, THF, 0-23° C., overnight) to give P-49. P-49 can be reacted with NaH in DMF/THF and a series of electrophiles such as (Mel, Etl, CF₃CH₂OTf) to give P-16 (where in this example R³=—CH₂—OR⁹) which can then further be reacted as in Scheme 26. The alcohol of P-49 can be oxidized, for example with MnO₂ in DCM at temperature ranging from rt to 40° C., to aldehyde P-50. P-50 can then be reacted with 2-methylpropane-2-sulfinamide in the presence of Ti(OiPr)₄ in toluene at rt followed by addition of Grignard reagents such as MeMgBr or other nucleophilic reagents such as TMSCF₃ to give P-51. The tBu(O)S— group can be removed using HCl (4 M in dioxane) with or without additional solvent such as methanol at rt. The resulting P-16 (where in this example R₃=—CH—(R⁸)—NH₂) can be either reacted as in Scheme 26 or the NH₂ group can be further reacted for example with either alkylating and or acylating reagents such as methyl trifluoromethanesulfonate (in ethyl acetate and sodium bicarbonate at rt) or bromobenzene (with Cs₂CO₃, X-Phos (CAS #564483-18-7) and Pd(OAc)₂ at 100° C. for 16 hours in toluene) to give P-16 (where in this further example R₃=—CH—(R⁸)—NR⁸R⁸) which can be reacted as in Scheme 26. P-50 can also be reacted with Grignard reagents such as MeMgBr in THF at temperatures ranging from −78° C. to rt, or can be oxidized to its methyl ester for example when using KOH and iodine in MeOH at room temperature (P-55) and then reacted with Grignard reagents such as MeMgBr in THF at temperatures ranging from −78° C. to rt to give secondary and/or tertiary alcohols as in P-52. P-52 can be further reacted with a base (such as NaH) and electrophiles such as Mel, Etl or CF₃CH₂OTf, or used as is to give P-16 (where in this example R₃=—CH—R⁸—OR⁹ or R₃=—C—(R⁸)(R⁸)—OR⁹ or R₃=—CH—(R⁸)—OH or R₃=CH—C—(R⁸)(R⁸)—OH) which can be reacted as in Scheme 26. P-50 can also be reacted with amines such as 1,1,1-trifluoropropan-2-amine with Ti(OiPr)₄ in toluene at temperatures ranging from rt to 45° C. followed by reduction to give P-16 (where in this example R₃=—CH₂—NHR⁸) which can be reacted as in Scheme 26. P-53 where for example R³ is CN (especially when X=Y=Z=R¹⁰=H and P″ is Br, CAS #1205515-06-5) can react with P-15 under typical Mitsunobu reaction conditions (such as PPh₃, DIAD, THF, 0-23° C., overnight) followed by CoCl₂ and NaBH₄ to give P-54. P-54 can then be reacted with aldehydes (or equivalents) such as 1-ethoxy-2,2,2-trifluoroethanol in MeOH at temperatures ranging from rt to 105° C. followed by NaBH₄ at temperatures ranging from rt to 50° C. The resulting amines can be used as is or be further alkylated as with P-51 to give P-16 (where in this example R₃=—CH₂—NHR⁸ or R₃=—CH₂—NR⁸R⁸) which can be reacted as in Scheme 26.

Compounds such as P-57 and P-59, when B (in the general formula) is CHCH₂CH₂OR⁹ or B is CHCH₂CH₂R⁸ can be accessed as described in Scheme 32.

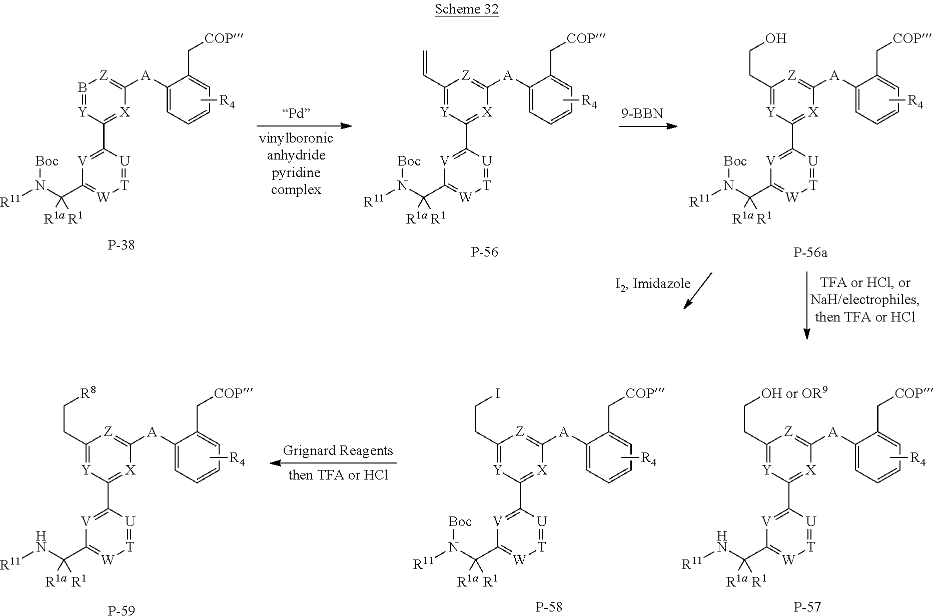

P-38 where, for example, B is CCl can be reacted with vinylboronic anhydride pyridine complex (CAS #442850-89-7) in solvent mixtures such as DME/H₂O with bases such as K₂CO₃ and palladium catalysts such as S-Phos palladacycle (CAS 1028206-58-7) heated in the microwave at temperature ranging from rt to 140° C. to give P-56. P-56 can then be reacted with a boron reagent such as 9-BBN in aprotic solvents such as THF at temperatures ranging from 0° C. to rt, followed by NaOH and H₂O₂ mixtures to provide P-56a. P-56a can be deprotected with TFA/DCM at temperature ranging from 0° C. to rt or HCl (4 M in dioxane) at temperature ranging from 0° C. to rt when P′″=O^{t}Bu, or with TFA/DCM at temperature ranging from 0° C. to rt and then LiOH in mixture of MeOH/THF/H₂O at temperature ranging from 0° C. to rt (or vice versa) when P′″=OMe to give P-57 (where P′″=OH). The alcohol of P-56a can be alkylated as shown in Scheme 31 for P-49 and then deprotected with TFA/DCM at temperatures ranging from 0° C. to rt or HCl (4 M in dioxane) at temperatures ranging from 0° C. to rt when P′″=O^{t}Bu, or with TFADCM at temperatures ranging from 0° C. to rt or HCl (4 M in dioxane) at temperatures ranging from 0° C. to rt and then LiOH in mixture of MeOH/THF/H₂O or CH₃CN at temperatures ranging from 0° C. to 60° C. (or vice versa) when P′″=OMe to give P-57 (where P′″=OH). The alcohol of P-56a can also be reacted with iodine/imidazole to give P-58 that can be reacted with a range of Grignard reagents such as cyclopropylmagnesium bromide to give compounds like P-59 after employing the same deprotection conditions as described for P-56.

Compounds such as P-63, when B (in the general formula) is CHCH₂CH₂CH₂F can be accessed as described in Scheme 33.

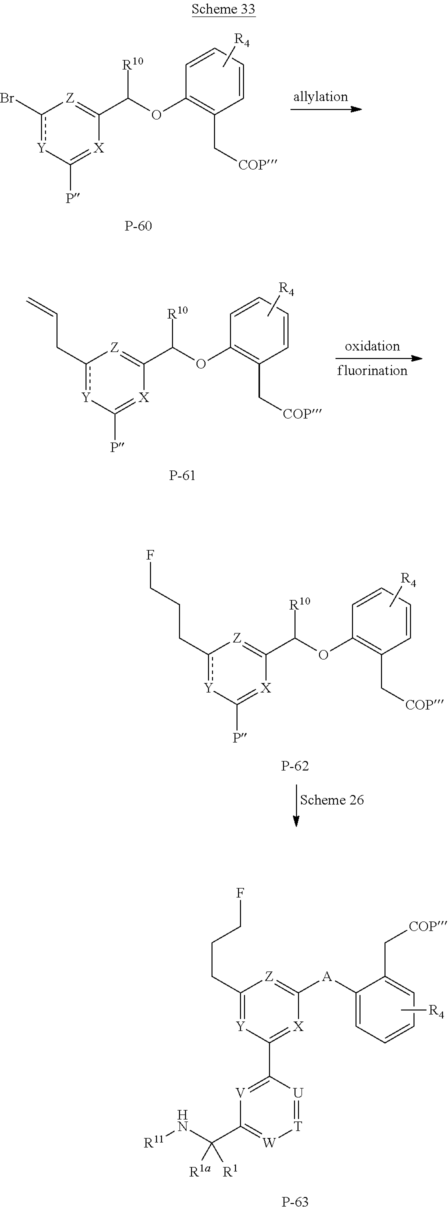

Compound such as P-60 (described above) can be reacted using Pd coupling and allyltributylstannane to afford compound such as P-61. Oxidation using 9-BBN at 0° C. followed by oxidative work-up (H₂O₂) gives rise to the wanted primary alcohol that can be transformed to the wanted fluoro derivative in P-62 using perfluoro-1-butanesulfonyl fluoride, diisopropylethylamine trihydrofluoride and diisopropylethylamine at 45° C. Using reaction depicted in Scheme 26, P-62 can be converted to the wanted P-63.

Compounds such as P-63, when A is —N(R¹⁶)CH₂ can be accessed as described in Scheme 34.

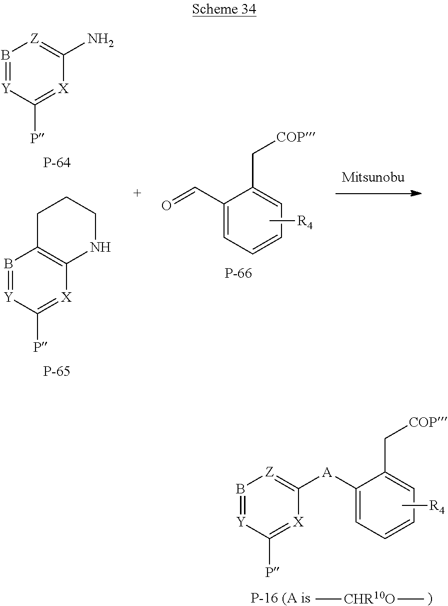

Anilines such as P-64 and P-65 can be reacted using standard reductive amination chemistry with aldehydes such as P-66 to access P-16.

Compounds such as P-63, when B is CR³ and R³ is C1-C6 alkyl wherein each group is optionally substituted by heterocycloalkyl or heteroaryl groups, can be accessed as described in Scheme 35.

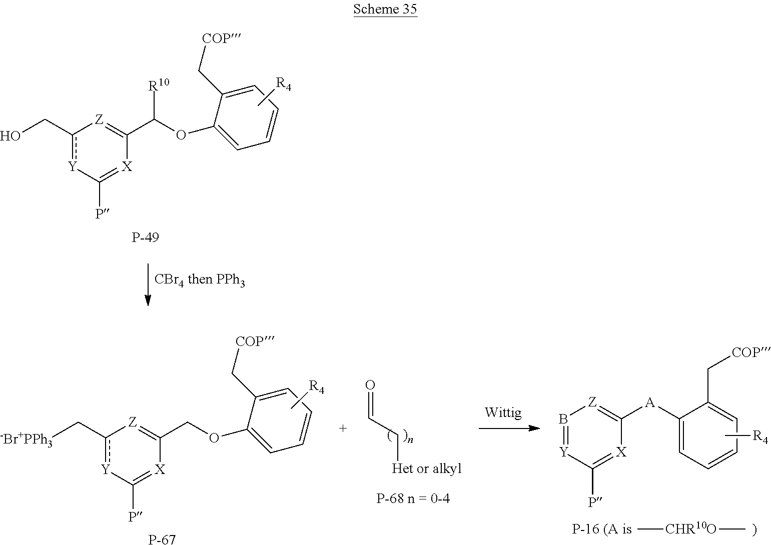

P-49 can be reacted with triphenylphosphine and CBr4 in an aprotic solvent such as DCM at room temperature to afford the benzylic bromide that can be transformed to the triphenylphosphinium salt P-67. Reaction of P-67 with an aldehyde of choice followed by Pd/C catalyzed hydrogenation can yield compounds like P-16.

Compounds such as P-16, when B is C—SO₂Me can be accessed as described in Scheme 36.

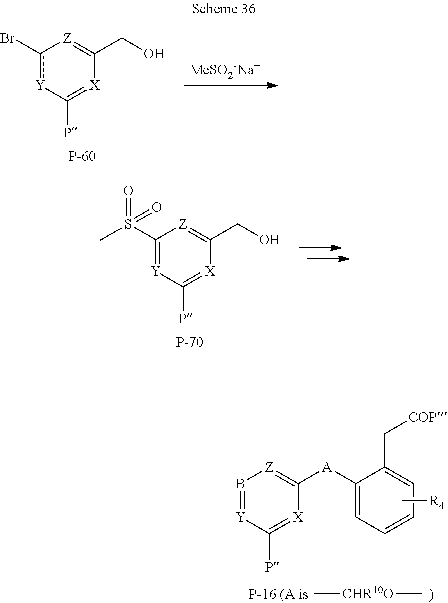

Compounds like P-69 can be reacted with copper(I) trifluoromethanesulfonate, methanesulfinic acid sodium salt and N,N-dimethyethylenediamine in DMSO (3.0 mL) at 110° C. to give compounds like P-70 which can be derived to P-16 following chemistry shown in the previous schemes.

In another aspect, the present invention provides a pharmaceutical composition comprising a compound of the present invention and a pharmaceutically acceptable carrier. The pharmaceutical composition can be formulated for particular routes of administration such as oral administration, parenteral administration, and ophthalmic administration, etc. In addition, the pharmaceutical compositions of the present invention can be made up in a solid form (including without limitation capsules, tablets, pills, granules, powders or suppositories), or in a liquid form (including without limitation solutions, suspensions, emulsions, each of which may be suitable for ophthalmic administration). The pharmaceutical compositions can be subjected to conventional pharmaceutical operations such as sterilization and/or can contain conventional inert diluents, lubricating agents, or buffering agents, as well as adjuvants, such as preservatives, stabilizers, wetting agents, emulsifers and buffers, etc.

Typically, the pharmaceutical compositions are tablets or gelatin capsules comprising the active ingredient together with
a) diluents, e.g., lactose, dextrose, sucrose, mannitol, sorbitol, cellulose and/or glycine;
b) lubricants, e.g., silica, talcum, stearic acid, its magnesium or calcium salt and/or polyethyleneglycol; for tablets also
c) binders, e.g., magnesium aluminum silicate, starch paste, gelatin, tragacanth, methylcellulose, sodium carboxymethylcellulose and/or polyvinylpyrrolidone; if desired
d) disintegrants, e.g., starches, agar, alginic acid or its sodium salt, or effervescent mixtures; and/or
e) absorbents, colorants, flavors and sweeteners.

Tablets may be either film coated or enteric coated according to methods known in the art.

Suitable compositions for oral administration include an effective amount of a compound of the invention in the form of tablets, lozenges, aqueous or oily suspensions, dispersible powders or granules, emulsion, hard or soft capsules, or syrups or elixirs. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets may contain the active ingredient in admixture with nontoxic pharmaceutically acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Certain injectable compositions are aqueous isotonic solutions or suspensions, and suppositories are advantageously prepared from fatty emulsions or suspensions. Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1-75%, or contain about 1-50%, of the active ingredient.

Suitable compositions for transdermal application include an effective amount of a compound of the invention with a suitable carrier. Carriers suitable for transdermal delivery include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound of the skin of the host at a controlled and predetermined rate over a prolonged period of time, and means to secure the device to the skin.

Suitable compositions for topical application, e.g., to the skin and eyes, include aqueous solutions, suspensions, ointments, creams, gels or sprayable formulations, e.g., for delivery by aerosol or the like. Such topical delivery systems will in particular be appropriate for ophthalmic application, e.g., for the treatment of eye diseases e.g., for therapeutic or prophylactic use in treating age related macular degeneration and other complement mediated ophthalmic disorders. Such may contain solubilizers, stabilizers, tonicity enhancing agents, buffers and preservatives.

As used herein a topical application may also pertain to an inhalation or to an intranasal application. They may be conveniently delivered in the form of a dry powder (either alone, as a mixture, for example a dry blend with lactose, or a mixed component particle, for example with phospholipids) from a dry powder inhaler or an aerosol spray presentation from a pressurised container, pump, spray, atomizer or nebuliser, with or without the use of a suitable propellant.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be desirable.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the active compound in a polymer matrix or gel.

Ophthalmic formulations, eye ointments, powders, solutions and the like, are also contemplated as being within the scope of this invention.

The present invention further provides anhydrous pharmaceutical compositions and dosage forms comprising the compounds of the present invention as active ingredients, since water may facilitate the degradation of certain compounds.

Anhydrous pharmaceutical compositions and dosage forms of the invention can be prepared using anhydrous or low moisture containing ingredients and low moisture or low humidity conditions. An anhydrous pharmaceutical composition may be prepared and stored such that its anhydrous nature is maintained. Accordingly, anhydrous compositions are packaged using materials known to prevent exposure to water such that they can be included in suitable formulary kits. Examples of suitable packaging include, but are not limited to, hermetically sealed foils, plastics, unit dose containers (e. g., vials), blister packs, and strip packs.

The invention further provides pharmaceutical compositions and dosage forms that comprise one or more agents that reduce the rate by which the compound of the present invention as an active ingredient will decompose. Such agents, which are referred to herein as “stabilizers,” include, but are not limited to, antioxidants such as ascorbic acid, pH buffers, or salt buffers, etc.

### Prophylactic and Therapeutic Uses

The compounds of formula I in free form or in pharmaceutically acceptable salt form, exhibit valuable pharmacological properties, e.g. Factor D modulating properties, complement pathway modulating properties and modulation of the complement alternative pathway properties, e.g. as indicated in in vitro and in vivo tests as provided in the next sections and are therefore indicated for therapy.

The present invention provides methods of treating a disease or disorder associated with increased complement activity by administering to a subject in need thereof an effective amount of the compounds of Formula (I) of the invention. In certain aspects, methods are provided for the treatment of diseases associated with increased activity of the C3 amplification loop of the complement pathway. In certain embodiments, methods of treating or preventing complement mediated diseases are provided in which the complement activation is induced by antibody-antigen interactions, by a component of an autoimmune disease, or by ischemic damage.

In a specific embodiment, the present invention provides a method of treating or preventing age-related macular degeneration (AMD) by administering to a subject in need thereof an effective amount of the compound of Formula (I) of the invention. In certain embodiments, patients who are currently asymptomatic but are at risk of developing a symptomatic macular degeneration related disorder are suitable for administration with a compound of the invention. The methods of treating or preventing AMD include, but are not limited to, methods of treating or preventing one or more symptoms or aspects of AMD selected from formation of ocular drusen, inflammation of the eye or eye tissue, loss of photoreceptor cells, loss of vision (including loss of visual acuity or visual field), neovascularization (including CNV), retinal detachment, photoreceptor degeneration, RPE degeneration, retinal degeneration, chorioretinal degeneration, cone degeneration, retinal dysfunction, retinal damage in response to light exposure, damage of the Bruch's membrane, and/or loss of RPE function.

The compound of Formula (I) of the invention can be used, inter alia, to prevent the onset of AMD, to prevent the progression of early AMD to advanced forms of AMD including neovascular AMD or geographic atrophy, to slow and/or prevent progression of geographic atrophy, to treat or prevent macular edema from AMD or other conditions (such as diabetic retinopathy, uveitis, or post surgical or non-surgical trauma), to prevent or reduce the loss of vision from AMD, and to improve vision lost due to pre-existing early or advanced AMD. It can also be used in combination with anti-VEGF therapies for the treatment of neovascular AMD patients or for the prevention of neovascular AMD. The present invention further provides methods of treating a complement related disease or disorder by administering to a subject in need thereof an effective amount of the compound(s) of the invention, wherein said disease or disorder is selected from uveitis, adult macular degeneration, diabetic retinopathy, retinitis pigmentosa, macular edema, Behcet's uveitis, multifocal choroiditis, Vogt-Koyangi-Harada syndrome, intermediate uveitis, birdshot retino-chorioditis, sympathetic ophthalmia, ocular dicatricial pemphigoid, ocular pemphigus, nonartertic ischemic optic neuropathy, post-operative inflammation, and retinal vein occlusion.

In some embodiments, the present invention provides methods of treating a complement related disease or disorder by administering to a subject in need thereof an effective amount of the compounds of the invention. Examples of known complement related diseases or disorders include: neurological disorders, multiple sclerosis, stroke, Guillain Barre Syndrome, traumatic brain injury, Parkinson's disease, disorders of inappropriate or undesirable complement activation, hemodialysis complications, hyperacute allograft rejection, xenograft rejection, interleukin-2 induced toxicity during IL-2 therapy, inflammatory disorders, inflammation of autoimmune diseases, Crohn's disease, adult respiratory distress syndrome, thermal injury including burns or frostbite, myocarditis, post-ischemic reperfusion conditions, myocardial infarction, balloon angioplasty, post-pump syndrome in cardiopulmonary bypass or renal bypass, atherosclerosis, hemodialysis, renal ischemia, mesenteric artery reperfusion after aortic reconstruction, infectious disease or sepsis, immune complex disorders and autoimmune diseases, rheumatoid arthritis, systemic lupus erythematosus (SLE), SLE nephritis, proliferative nephritis, liver fibrosis, hemolytic anemia, myasthenia gravis, tissue regeneration and neural regeneration. In addition, other known complement related disease are lung disease and disorders such as dyspnea, hemoptysis, ARDS, asthma, chronic obstructive pulmonary disease (COPD), emphysema, pulmonary embolisms and infarcts, pneumonia, fibrogenic dust diseases, inert dusts and minerals (e.g., silicon, coal dust, beryllium, and asbestos), pulmonary fibrosis, organic dust diseases, chemical injury (due to irritant gases and chemicals, e.g., chlorine, phosgene, sulfur dioxide, hydrogen sulfide, nitrogen dioxide, ammonia, and hydrochloric acid), smoke injury, thermal injury (e.g., burn, freeze), asthma, allergy, bronchoconstriction, hypersensitivity pneumonitis, parasitic diseases, Goodpasture's Syndrome, pulmonary vasculitis, Pauci-immune vasculitis, immune complex-associated inflammation, uveitis (including Behcet's disease and other sub-types of uveitis), antiphospholipid syndrome.

In a specific embodiment, the present invention provides methods of treating a complement related disease or disorder by administering to a subject in need thereof an effective amount of the compounds of the invention, wherein said disease or disorder is asthma, arthritis (e.g., rheumatoid arthritis), autoimmune heart disease, multiple sclerosis, inflammatory bowel disease, ischemia-reperfusion injuries, Barraquer-Simons Syndrome, hemodialysis, systemic lupus, lupus erythematosus, psoriasis, multiple sclerosis, transplantation, diseases of the central nervous system such as Alzheimer's disease and other neurodegenerative conditions, atypicaly hemolytic uremic syndrome (aHUS), glomerulonephritis (including membrane proliferative glomerulonephritis), blistering cutaneous diseases (including bullous pemphigoid, pemphigus, and epidermolysis bullosa), ocular cicatrical pemphigoid or MPGN II.

In a specific embodiment, the present invention provides methods of treating glomerulonephritis by administering to a subject in need thereof an effective amount of a composition comprising a compound of the present invention. Symptoms of glomerulonephritis include, but not limited to, proteinuria; reduced glomerular filtration rate (GFR); serum electrolyte changes including azotemia (uremia, excessive blood urea nitrogen—BUN) and salt retention, leading to water retention resulting in hypertension and edema; hematuria and abnormal urinary sediments including red cell casts; hypoalbuminemia; hyperlipidemia; and lipiduria. In a specific embodiment, the present invention provides methods of treating paroxysmal nocturnal hemoglobinuria (PNH) by administering to a subject in need thereof an effective amount of a composition comprising an compound of the present invention with or without concomitent administration of a complement C5 inhibitor or C5 convertase inhibitor such as Soliris.

In a specific embodiment, the present invention provides methods of reducing the dysfunction of the immune and/or hemostatic systems associated with extracorporeal circulation by administering to a subject in need thereof an effective amount of a composition comprising an compound of the present invention. The compounds of the present invention can be used in any procedure which involves circulating the patient's blood from a blood vessel of the patient, through a conduit, and back to a blood vessel of the patient, the conduit having a luminal surface comprising a material capable of causing at least one of complement activation, platelet activation, leukocyte activation, or platelet-leukocyte adhesion. Such procedures include, but are not limited to, all forms of ECC, as well as procedures involving the introduction of an artificial or foreign organ, tissue, or vessel into the blood circuit of a patient. More particularly, such procedures include, but are not limited to, transplantation procedures including kidney, liver, lung or heart transplant procedures and islet cell transplant procedures.

In other embodiments, the compounds of the invention are suitable for use in the treatment of diseases and disorders associated with fatty acid metabolism, including obesity and other metabolic disorders.

In one embodiment of the present invention, (R)-2-(2-((3′-(1-Aminoethyl)-5-(hydroxymethyl)-[1,1′-biphenyl]-3-yl)methoxy)phenyl)acetic acid is provided for use in the treatment of a disorder or a disease in a subject mediated by complement activation, in particular mediated by activation of the complement alternative pathway. In certain embodiments, the disease or disorder mediated by complement activation is selected from age-related macular degeneration, geographic atrophy, diabetic retinopathy, uveitis, retinitis pigmentosa, macular edema, Behcet's uveitis, multifocal choroiditis, Vogt-Koyangi-Harada syndrome, intermediate uveitis, birdshot retino-chorioditis, sympathetic ophthalmia, ocular dicatricial pemphigoid, ocular pemphigus, nonartertic ischemic optic neuropathy, post-operative inflammation, retinal vein occlusion, neurological disorders, multiple sclerosis, stroke, Guillain Barre Syndrome, traumatic brain injury, Parkinson's disease, disorders of inappropriate or undesirable complement activation, hemodialysis complications, hyperacute allograft rejection, xenograft rejection, interleukin-2 induced toxicity during IL-2 therapy, inflammatory disorders, inflammation of autoimmune diseases, Crohn's disease, adult respiratory distress syndrome, myocarditis, post-ischemic reperfusion conditions, myocardial infarction, balloon angioplasty, post-pump syndrome in cardiopulmonary bypass or renal bypass, atherosclerosis, hemodialysis, renal ischemia, mesenteric artery reperfusion after aortic reconstruction, infectious disease or sepsis, immune complex disorders and autoimmune diseases, rheumatoid arthritis, systemic lupus erythematosus (SLE), SLE nephritis, proliferative nephritis, liver fibrosis, hemolytic anemia, myasthenia gravis, tissue regeneration, neural regeneration, dyspnea, hemoptysis, ARDS, asthma, chronic obstructive pulmonary disease (COPD), emphysema, pulmonary embolisms and infarcts, pneumonia, fibrogenic dust diseases, pulmonary fibrosis, asthma, allergy, bronchoconstriction, hypersensitivity pneumonitis, parasitic diseases, Goodpasture's Syndrome, pulmonary vasculitis, Pauci-immune vasculitis, immune complex-associated inflammation, antiphospholipid syndrome, glomerulonephritis and obesity. In certain preferred embodiments, the disease or disorder mediated by complement activation is selected from age-related macular degeneration, geographic atrophy, diabetic retinopathy, uveitis, retinitis pigmentosa, or macular edema.

In one embodiment of the present invention, (S)-2-(2-((3′-(1-Amino-2-hydroxyethyl)-[1,1′-biphenyl]-3-yl)methoxy)phenyl)acetic acid is provided for use in the treatment of a disorder or a disease in a subject mediated by complement activation, in particular mediated by activation of the complement alternative pathway. In certain embodiments, the disease or disorder mediated by complement activation is selected from age-related macular degeneration, geographic atrophy, diabetic retinopathy, uveitis, retinitis pigmentosa, macular edema, Behcet's uveitis, multifocal choroiditis, Vogt-Koyangi-Harada syndrome, intermediate uveitis, birdshot retino-chorioditis, sympathetic ophthalmia, ocular dicatricial pemphigoid, ocular pemphigus, nonartertic ischemic optic neuropathy, post-operative inflammation, retinal vein occlusion, neurological disorders, multiple sclerosis, stroke, Guillain Barre Syndrome, traumatic brain injury, Parkinson's disease, disorders of inappropriate or undesirable complement activation, hemodialysis complications, hyperacute allograft rejection, xenograft rejection, interleukin-2 induced toxicity during IL-2 therapy, inflammatory disorders, inflammation of autoimmune diseases, Crohn's disease, adult respiratory distress syndrome, myocarditis, post-ischemic reperfusion conditions, myocardial infarction, balloon angioplasty, post-pump syndrome in cardiopulmonary bypass or renal bypass, atherosclerosis, hemodialysis, renal ischemia, mesenteric artery reperfusion after aortic reconstruction, infectious disease or sepsis, immune complex disorders and autoimmune diseases, rheumatoid arthritis, systemic lupus erythematosus (SLE), SLE nephritis, proliferative nephritis, liver fibrosis, hemolytic anemia, myasthenia gravis, tissue regeneration, neural regeneration, dyspnea, hemoptysis, ARDS, asthma, chronic obstructive pulmonary disease (COPD), emphysema, pulmonary embolisms and infarcts, pneumonia, fibrogenic dust diseases, pulmonary fibrosis, asthma, allergy, bronchoconstriction, hypersensitivity pneumonitis, parasitic diseases, Goodpasture's Syndrome, pulmonary vasculitis, Pauci-immune vasculitis, immune complex-associated inflammation, antiphospholipid syndrome, glomerulonephritis and obesity. In certain preferred embodiments, the disease or disorder mediated by complement activation is selected from age-related macular degeneration, geographic atrophy, diabetic retinopathy, uveitis, retinitis pigmentosa, or macular edema.

In one embodiment of the present invention, (S)-2-(2-((3′-(1-Amino-2-hydroxyethyl)-5-ethoxy-[1,1′-biphenyl]-3-yl)methoxy)phenyl)acetic acid is provided for use in the treatment of a disorder or a disease in a subject mediated by complement activation, in particular mediated by activation of the complement alternative pathway. In certain embodiments, the disease or disorder mediated by complement activation is selected from age-related macular degeneration, geographic atrophy, diabetic retinopathy, uveitis, retinitis pigmentosa, macular edema, Behcet's uveitis, multifocal choroiditis, Vogt-Koyangi-Harada syndrome, intermediate uveitis, birdshot retino-chorioditis, sympathetic ophthalmia, ocular dicatricial pemphigoid, ocular pemphigus, nonartertic ischemic optic neuropathy, post-operative inflammation, retinal vein occlusion, neurological disorders, multiple sclerosis, stroke, Guillain Barre Syndrome, traumatic brain injury, Parkinson's disease, disorders of inappropriate or undesirable complement activation, hemodialysis complications, hyperacute allograft rejection, xenograft rejection, interleukin-2 induced toxicity during IL-2 therapy, inflammatory disorders, inflammation of autoimmune diseases, Crohn's disease, adult respiratory distress syndrome, myocarditis, post-ischemic reperfusion conditions, myocardial infarction, balloon angioplasty, post-pump syndrome in cardiopulmonary bypass or renal bypass, atherosclerosis, hemodialysis, renal ischemia, mesenteric artery reperfusion after aortic reconstruction, infectious disease or sepsis, immune complex disorders and autoimmune diseases, rheumatoid arthritis, systemic lupus erythematosus (SLE), SLE nephritis, proliferative nephritis, liver fibrosis, hemolytic anemia, myasthenia gravis, tissue regeneration, neural regeneration, dyspnea, hemoptysis, ARDS, asthma, chronic obstructive pulmonary disease (COPD), emphysema, pulmonary embolisms and infarcts, pneumonia, fibrogenic dust diseases, pulmonary fibrosis, asthma, allergy, bronchoconstriction, hypersensitivity pneumonitis, parasitic diseases, Goodpasture's Syndrome, pulmonary vasculitis, Pauci-immune vasculitis, immune complex-associated inflammation, antiphospholipid syndrome, glomerulonephritis and obesity. In certain preferred embodiments, the disease or disorder mediated by complement activation is selected from age-related macular degeneration, geographic atrophy, diabetic retinopathy, uveitis, retinitis pigmentosa, or macular edema.

In one embodiment of the present invention, (S)-2-(2-((3′-(1-Amino-2-hydroxyethyl)-5-(2-hydroxypropan-2-yl)-[1,1′-biphenyl]-3-yl)methoxy)phenyl)acetic acid is provided for use in the treatment of a disorder or a disease in a subject mediated by complement activation, in particular mediated by activation of the complement alternative pathway. In certain embodiments, the disease or disorder mediated by complement activation is selected from age-related macular degeneration, geographic atrophy, diabetic retinopathy, uveitis, retinitis pigmentosa, macular edema, Behcet's uveitis, multifocal choroiditis, Vogt-Koyangi-Harada syndrome, intermediate uveitis, birdshot retino-chorioditis, sympathetic ophthalmia, ocular dicatricial pemphigoid, ocular pemphigus, nonartertic ischemic optic neuropathy, post-operative inflammation, retinal vein occlusion, neurological disorders, multiple sclerosis, stroke, Guillain Barre Syndrome, traumatic brain injury, Parkinson's disease, disorders of inappropriate or undesirable complement activation, hemodialysis complications, hyperacute allograft rejection, xenograft rejection, interleukin-2 induced toxicity during IL-2 therapy, inflammatory disorders, inflammation of autoimmune diseases, Crohn's disease, adult respiratory distress syndrome, myocarditis, post-ischemic reperfusion conditions, myocardial infarction, balloon angioplasty, post-pump syndrome in cardiopulmonary bypass or renal bypass, atherosclerosis, hemodialysis, renal ischemia, mesenteric artery reperfusion after aortic reconstruction, infectious disease or sepsis, immune complex disorders and autoimmune diseases, rheumatoid arthritis, systemic lupus erythematosus (SLE), SLE nephritis, proliferative nephritis, liver fibrosis, hemolytic anemia, myasthenia gravis, tissue regeneration, neural regeneration, dyspnea, hemoptysis, ARDS, asthma, chronic obstructive pulmonary disease (COPD), emphysema, pulmonary embolisms and infarcts, pneumonia, fibrogenic dust diseases, pulmonary fibrosis, asthma, allergy, bronchoconstriction, hypersensitivity pneumonitis, parasitic diseases, Goodpasture's Syndrome, pulmonary vasculitis, Pauci-immune vasculitis, immune complex-associated inflammation, antiphospholipid syndrome, glomerulonephritis and obesity. In certain preferred embodiments, the disease or disorder mediated by complement activation is selected from age-related macular degeneration, geographic atrophy, diabetic retinopathy, uveitis, retinitis pigmentosa, or macular edema.

In one embodiment of the present invention, (R)-2-(2-((3′-(1-Aminoethyl)-2′-fluoro-5-(methoxymethyl)-[1,1′-biphenyl]-3-yl)methoxy)phenyl)acetic acid is provided for use in the treatment of a disorder or a disease in a subject mediated by complement activation, in particular mediated by activation of the complement alternative pathway. In certain embodiments, the disease or disorder mediated by complement activation is selected from age-related macular degeneration, geographic atrophy, diabetic retinopathy, uveitis, retinitis pigmentosa, macular edema, Behcet's uveitis, multifocal choroiditis, Vogt-Koyangi-Harada syndrome, intermediate uveitis, birdshot retino-chorioditis, sympathetic ophthalmia, ocular dicatricial pemphigoid, ocular pemphigus, nonartertic ischemic optic neuropathy, post-operative inflammation, retinal vein occlusion, neurological disorders, multiple sclerosis, stroke, Guillain Barre Syndrome, traumatic brain injury, Parkinson's disease, disorders of inappropriate or undesirable complement activation, hemodialysis complications, hyperacute allograft rejection, xenograft rejection, interleukin-2 induced toxicity during IL-2 therapy, inflammatory disorders, inflammation of autoimmune diseases, Crohn's disease, adult respiratory distress syndrome, myocarditis, post-ischemic reperfusion conditions, myocardial infarction, balloon angioplasty, post-pump syndrome in cardiopulmonary bypass or renal bypass, atherosclerosis, hemodialysis, renal ischemia, mesenteric artery reperfusion after aortic reconstruction, infectious disease or sepsis, immune complex disorders and autoimmune diseases, rheumatoid arthritis, systemic lupus erythematosus (SLE), SLE nephritis, proliferative nephritis, liver fibrosis, hemolytic anemia, myasthenia gravis, tissue regeneration, neural regeneration, dyspnea, hemoptysis, ARDS, asthma, chronic obstructive pulmonary disease (COPD), emphysema, pulmonary embolisms and infarcts, pneumonia, fibrogenic dust diseases, pulmonary fibrosis, asthma, allergy, bronchoconstriction, hypersensitivity pneumonitis, parasitic diseases, Goodpasture's Syndrome, pulmonary vasculitis, Pauci-immune vasculitis, immune complex-associated inflammation, antiphospholipid syndrome, glomerulonephritis and obesity. In certain preferred embodiments, the disease or disorder mediated by complement activation is selected from age-related macular degeneration, geographic atrophy, diabetic retinopathy, uveitis, retinitis pigmentosa, or macular edema.

In one embodiment of the present invention, (R)-2-(2-((3′-(1-Aminoethyl)-5-(methoxymethyl)-[1,1′-biphenyl]-3-yl)methoxy)phenyl)acetic acid is provided for use in the treatment of a disorder or a disease in a subject mediated by complement activation, in particular mediated by activation of the complement alternative pathway. In certain embodiments, the disease or disorder mediated by complement activation is selected from age-related macular degeneration, geographic atrophy, diabetic retinopathy, uveitis, retinitis pigmentosa, macular edema, Behcet's uveitis, multifocal choroiditis, Vogt-Koyangi-Harada syndrome, intermediate uveitis, birdshot retino-chorioditis, sympathetic ophthalmia, ocular dicatricial pemphigoid, ocular pemphigus, nonartertic ischemic optic neuropathy, post-operative inflammation, retinal vein occlusion, neurological disorders, multiple sclerosis, stroke, Guillain Barre Syndrome, traumatic brain injury, Parkinson's disease, disorders of inappropriate or undesirable complement activation, hemodialysis complications, hyperacute allograft rejection, xenograft rejection, interleukin-2 induced toxicity during IL-2 therapy, inflammatory disorders, inflammation of autoimmune diseases, Crohn's disease, adult respiratory distress syndrome, myocarditis, post-ischemic reperfusion conditions, myocardial infarction, balloon angioplasty, post-pump syndrome in cardiopulmonary bypass or renal bypass, atherosclerosis, hemodialysis, renal ischemia, mesenteric artery reperfusion after aortic reconstruction, infectious disease or sepsis, immune complex disorders and autoimmune diseases, rheumatoid arthritis, systemic lupus erythematosus (SLE), SLE nephritis, proliferative nephritis, liver fibrosis, hemolytic anemia, myasthenia gravis, tissue regeneration, neural regeneration, dyspnea, hemoptysis, ARDS, asthma, chronic obstructive pulmonary disease (COPD), emphysema, pulmonary embolisms and infarcts, pneumonia, fibrogenic dust diseases, pulmonary fibrosis, asthma, allergy, bronchoconstriction, hypersensitivity pneumonitis, parasitic diseases, Goodpasture's Syndrome, pulmonary vasculitis, Pauci-immune vasculitis, immune complex-associated inflammation, antiphospholipid syndrome, glomerulonephritis and obesity. In certain preferred embodiments, the disease or disorder mediated by complement activation is selected from age-related macular degeneration, geographic atrophy, diabetic retinopathy, uveitis, retinitis pigmentosa, or macular edema.

In one embodiment of the present invention, (S)-2-(2-((3′-(1-Amino-2-hydroxyethyl)-5-((cyclopropylmethyl)amino)-[1,1′-biphenyl]-3-yl)methoxy)phenyl)acetic acid is provided for use in the treatment of a disorder or a disease in a subject mediated by complement activation, in particular mediated by activation of the complement alternative pathway. In certain embodiments, the disease or disorder mediated by complement activation is selected from age-related macular degeneration, geographic atrophy, diabetic retinopathy, uveitis, retinitis pigmentosa, macular edema, Behcet's uveitis, multifocal choroiditis, Vogt-Koyangi-Harada syndrome, intermediate uveitis, birdshot retino-chorioditis, sympathetic ophthalmia, ocular dicatricial pemphigoid, ocular pemphigus, nonartertic ischemic optic neuropathy, post-operative inflammation, retinal vein occlusion, neurological disorders, multiple sclerosis, stroke, Guillain Barre Syndrome, traumatic brain injury, Parkinson's disease, disorders of inappropriate or undesirable complement activation, hemodialysis complications, hyperacute allograft rejection, xenograft rejection, interleukin-2 induced toxicity during IL-2 therapy, inflammatory disorders, inflammation of autoimmune diseases, Crohn's disease, adult respiratory distress syndrome, myocarditis, post-ischemic reperfusion conditions, myocardial infarction, balloon angioplasty, post-pump syndrome in cardiopulmonary bypass or renal bypass, atherosclerosis, hemodialysis, renal ischemia, mesenteric artery reperfusion after aortic reconstruction, infectious disease or sepsis, immune complex disorders and autoimmune diseases, rheumatoid arthritis, systemic lupus erythematosus (SLE), SLE nephritis, proliferative nephritis, liver fibrosis, hemolytic anemia, myasthenia gravis, tissue regeneration, neural regeneration, dyspnea, hemoptysis, ARDS, asthma, chronic obstructive pulmonary disease (COPD), emphysema, pulmonary embolisms and infarcts, pneumonia, fibrogenic dust diseases, pulmonary fibrosis, asthma, allergy, bronchoconstriction, hypersensitivity pneumonitis, parasitic diseases, Goodpasture's Syndrome, pulmonary vasculitis, Pauci-immune vasculitis, immune complex-associated inflammation, antiphospholipid syndrome, glomerulonephritis and obesity. In certain preferred embodiments, the disease or disorder mediated by complement activation is selected from age-related macular degeneration, geographic atrophy, diabetic retinopathy, uveitis, retinitis pigmentosa, or macular edema.

In one embodiment of the present invention, (S)-2-(2-((3′-(Aminomethyl)-5-(((tetrahydrofuran-2-yl)methyl)amino)-[1,1′-biphenyl]-3-yl)methoxy)phenyl)acetic acid is provided for use in the treatment of a disorder or a disease in a subject mediated by complement activation, in particular mediated by activation of the complement alternative pathway. In certain embodiments, the disease or disorder mediated by complement activation is selected from age-related macular degeneration, geographic atrophy, diabetic retinopathy, uveitis, retinitis pigmentosa, macular edema, Behcet's uveitis, multifocal choroiditis, Vogt-Koyangi-Harada syndrome, intermediate uveitis, birdshot retino-chorioditis, sympathetic ophthalmia, ocular dicatricial pemphigoid, ocular pemphigus, nonartertic ischemic optic neuropathy, post-operative inflammation, retinal vein occlusion, neurological disorders, multiple sclerosis, stroke, Guillain Barre Syndrome, traumatic brain injury, Parkinson's disease, disorders of inappropriate or undesirable complement activation, hemodialysis complications, hyperacute allograft rejection, xenograft rejection, interleukin-2 induced toxicity during IL-2 therapy, inflammatory disorders, inflammation of autoimmune diseases, Crohn's disease, adult respiratory distress syndrome, myocarditis, post-ischemic reperfusion conditions, myocardial infarction, balloon angioplasty, post-pump syndrome in cardiopulmonary bypass or renal bypass, atherosclerosis, hemodialysis, renal ischemia, mesenteric artery reperfusion after aortic reconstruction, infectious disease or sepsis, immune complex disorders and autoimmune diseases, rheumatoid arthritis, systemic lupus erythematosus (SLE), SLE nephritis, proliferative nephritis, liver fibrosis, hemolytic anemia, myasthenia gravis, tissue regeneration, neural regeneration, dyspnea, hemoptysis, ARDS, asthma, chronic obstructive pulmonary disease (COPD), emphysema, pulmonary embolisms and infarcts, pneumonia, fibrogenic dust diseases, pulmonary fibrosis, asthma, allergy, bronchoconstriction, hypersensitivity pneumonitis, parasitic diseases, Goodpasture's Syndrome, pulmonary vasculitis, Pauci-immune vasculitis, immune complex-associated inflammation, antiphospholipid syndrome, glomerulonephritis and obesity. In certain preferred embodiments, the disease or disorder mediated by complement activation is selected from age-related macular degeneration, geographic atrophy, diabetic retinopathy, uveitis, retinitis pigmentosa, or macular edema.

In one embodiment of the present invention, (R)-2-(2-((3′-(1-aminoethyl)-5-(ethylamino)-2′-fluoro-[1,1′-biphenyl]-3-yl)methoxy)phenyl)acetic acid is provided for use in the treatment of a disorder or a disease in a subject mediated by complement activation, in particular mediated by activation of the complement alternative pathway. In certain embodiments, the disease or disorder mediated by complement activation is selected from age-related macular degeneration, geographic atrophy, diabetic retinopathy, uveitis, retinitis pigmentosa, macular edema, Behcet's uveitis, multifocal choroiditis, Vogt-Koyangi-Harada syndrome, intermediate uveitis, birdshot retino-chorioditis, sympathetic ophthalmia, ocular dicatricial pemphigoid, ocular pemphigus, nonartertic ischemic optic neuropathy, post-operative inflammation, retinal vein occlusion, neurological disorders, multiple sclerosis, stroke, Guillain Barre Syndrome, traumatic brain injury, Parkinson's disease, disorders of inappropriate or undesirable complement activation, hemodialysis complications, hyperacute allograft rejection, xenograft rejection, interleukin-2 induced toxicity during IL-2 therapy, inflammatory disorders, inflammation of autoimmune diseases, Crohn's disease, adult respiratory distress syndrome, myocarditis, post-ischemic reperfusion conditions, myocardial infarction, balloon angioplasty, post-pump syndrome in cardiopulmonary bypass or renal bypass, atherosclerosis, hemodialysis, renal ischemia, mesenteric artery reperfusion after aortic reconstruction, infectious disease or sepsis, immune complex disorders and autoimmune diseases, rheumatoid arthritis, systemic lupus erythematosus (SLE), SLE nephritis, proliferative nephritis, liver fibrosis, hemolytic anemia, myasthenia gravis, tissue regeneration, neural regeneration, dyspnea, hemoptysis, ARDS, asthma, chronic obstructive pulmonary disease (COPD), emphysema, pulmonary embolisms and infarcts, pneumonia, fibrogenic dust diseases, pulmonary fibrosis, asthma, allergy, bronchoconstriction, hypersensitivity pneumonitis, parasitic diseases, Goodpasture's Syndrome, pulmonary vasculitis, Pauci-immune vasculitis, immune complex-associated inflammation, antiphospholipid syndrome, glomerulonephritis and obesity. In certain preferred embodiments, the disease or disorder mediated by complement activation is selected from age-related macular degeneration, geographic atrophy, diabetic retinopathy, uveitis, retinitis pigmentosa, or macular edema.

In one embodiment of the present invention, 2-(2-((6-(3-(Aminomethyl)phenyl)-1H-indazol-4-yl)methoxy)phenyl)acetic acid is provided for use in the treatment of a disorder or a disease in a subject mediated by complement activation, in particular mediated by activation of the complement alternative pathway. In certain embodiments, the disease or disorder mediated by complement activation is selected from age-related macular degeneration, geographic atrophy, diabetic retinopathy, uveitis, retinitis pigmentosa, macular edema, Behcet's uveitis, multifocal choroiditis, Vogt-Koyangi-Harada syndrome, intermediate uveitis, birdshot retino-chorioditis, sympathetic ophthalmia, ocular dicatricial pemphigoid, ocular pemphigus, nonartertic ischemic optic neuropathy, post-operative inflammation, retinal vein occlusion, neurological disorders, multiple sclerosis, stroke, Guillain Barre Syndrome, traumatic brain injury, Parkinson's disease, disorders of inappropriate or undesirable complement activation, hemodialysis complications, hyperacute allograft rejection, xenograft rejection, interleukin-2 induced toxicity during IL-2 therapy, inflammatory disorders, inflammation of autoimmune diseases, Crohn's disease, adult respiratory distress syndrome, myocarditis, post-ischemic reperfusion conditions, myocardial infarction, balloon angioplasty, post-pump syndrome in cardiopulmonary bypass or renal bypass, atherosclerosis, hemodialysis, renal ischemia, mesenteric artery reperfusion after aortic reconstruction, infectious disease or sepsis, immune complex disorders and autoimmune diseases, rheumatoid arthritis, systemic lupus erythematosus (SLE), SLE nephritis, proliferative nephritis, liver fibrosis, hemolytic anemia, myasthenia gravis, tissue regeneration, neural regeneration, dyspnea, hemoptysis, ARDS, asthma, chronic obstructive pulmonary disease (COPD), emphysema, pulmonary embolisms and infarcts, pneumonia, fibrogenic dust diseases, pulmonary fibrosis, asthma, allergy, bronchoconstriction, hypersensitivity pneumonitis, parasitic diseases, Goodpasture's Syndrome, pulmonary vasculitis, Pauci-immune vasculitis, immune complex-associated inflammation, antiphospholipid syndrome, glomerulonephritis and obesity. In certain preferred embodiments, the disease or disorder mediated by complement activation is selected from age-related macular degeneration, geographic atrophy, diabetic retinopathy, uveitis, retinitis pigmentosa, or macular edema.

In another embodiment, the compounds of the invention may be used in blood ampules, diagnostic kits and other equipment used in the collection and sampling of blood. The use of the compounds of the invention in such diagnostic kits may inhibit the ex vivo activation of the complement pathway associated with blood sampling.

The pharmaceutical composition or combination of the present invention can be in unit dosage of about 1-1000 mg of active ingredient(s) for a subject of about 50-70 kg, or about 1-500 mg or about 1-250 mg or about 1-150 mg or about 0.5-100 mg, or about 1-50 mg of active ingredients. The therapeutically effective dosage of a compound, the pharmaceutical composition, or the combinations thereof, is dependent on the species of the subject, the body weight, age and individual condition, the disorder or disease or the severity thereof being treated. A physician, clinician or veterinarian of ordinary skill can readily determine the effective amount of each of the active ingredients necessary to prevent, treat or inhibit the progress of the disorder or disease.

The above-cited dosage properties are demonstrable in vitro and in vivo tests using advantageously mammals, e.g., mice, rats, dogs, monkeys or isolated organs, tissues and preparations thereof. The compounds of the present invention can be applied in vitro in the form of solutions, e.g., aqueous solutions, and in vivo either enterally, parenterally, advantageously intravenously, e.g., as a suspension or in aqueous solution. The dosage in vitro may range between about 10⁻³ molar and 10⁻⁹ molar concentrations. A therapeutically effective amount in vivo may range depending on the route of administration, between about 0.1-500 mg/kg, or between about 1-100 mg/kg.

The activity of a compound according to the present invention can be assessed by the following in vitro & in vivo methods.

The compound of the present invention may be administered either simultaneously with, or before or after, one or more other therapeutic agent. The compound of the present invention may be administered separately, by the same or different route of administration, or together in the same pharmaceutical composition as the other agents.

In one embodiment, the invention provides a product comprising a compound of formula (I) and at least one other therapeutic agent as a combined preparation for simultaneous, separate or sequential use in therapy. In one embodiment, the therapy is the treatment of a disease or condition mediated by alternative complement pathway. Products provided as a combined preparation include a composition comprising the compound of formula (I) and the other therapeutic agent(s) together in the same pharmaceutical composition, or the compound of formula (I) and the other therapeutic agent(s) in separate form, e.g. in the form of a kit.

In one embodiment, the invention provides a pharmaceutical composition comprising a compound of formula (I) and another therapeutic agent(s). Optionally, the pharmaceutical composition may comprise a pharmaceutically acceptable excipient, as described above.

In one embodiment, the invention provides a kit comprising two or more separate pharmaceutical compositions, at least one of which contains a compound of formula (I). In one embodiment, the kit comprises means for separately retaining said compositions, such as a container, divided bottle, or divided foil packet. An example of such a kit is a blister pack, as typically used for the packaging of tablets, capsules and the like.

The kit of the invention may be used for administering different dosage forms, for example, oral and parenteral, for administering the separate compositions at different dosage intervals, or for titrating the separate compositions against one another. To assist compliance, the kit of the invention typically comprises directions for administration.

In the combination therapies of the invention, the compound of the invention and the other therapeutic agent may be manufactured and/or formulated by the same or different manufacturers. Moreover, the compound of the invention and the other therapeutic may be brought together into a combination therapy: (i) prior to release of the combination product to physicians (e.g. in the case of a kit comprising the compound of the invention and the other therapeutic agent); (ii) by the physician themselves (or under the guidance of the physician) shortly before administration; (iii) in the patient themselves, e.g. during sequential administration of the compound of the invention and the other therapeutic agent.

Accordingly, the invention provides the use of a compound of formula (I) for treating a disease or condition mediated by the complement alternative pathway, wherein the medicament is prepared for administration with another therapeutic agent. The invention also provides the use of another therapeutic agent for treating a disease or condition mediated by the complement alternative pathway, wherein the medicament is administered with a compound of formula (I).

The invention also provides a compound of formula (I) for use in a method of treating a disease or condition mediated by the complement alternative pathway, wherein the compound of formula (I) is prepared for administration with another therapeutic agent. The invention also provides another therapeutic agent for use in a method of treating a disease or condition mediated by the complement alternative pathway and/or Factor D, wherein the other therapeutic agent is prepared for administration with a compound of formula (I). The invention also provides a compound of formula (I) for use in a method of treating a disease or condition mediated by the complement alternative pathway and/or Factor D, wherein the compound of formula (I) is administered with another therapeutic agent. The invention also provides another therapeutic agent for use in a method of treating a disease or condition mediated by the complement alternative pathway and/or Factor D, wherein the other therapeutic agent is administered with a compound of formula (I).

The invention also provides the use of a compound of formula (I) for treating a disease or condition mediated by the complement alternative pathway and/or Factor D, wherein the patient has previously (e.g. within 24 hours) been treated with another therapeutic agent. The invention also provides the use of another therapeutic agent for treating a disease or condition mediated by the complement alternative pathway and/or Factor D wherein the patient has previously (e.g. within 24 hours) been treated with a compound of formula (I).

The pharmaceutical compositions can be administered alone or in combination with other molecules known to have a beneficial effect on retinal attachment or damaged retinal tissue, including molecules capable of tissue repair and regeneration and/or inhibiting inflammation. Examples of useful, cofactors include anti-VEGF agents (such as an antibody or FAB against VEGF, e.g., Lucentis or Avastin), basic fibroblast growth factor (bFGF), ciliary neurotrophic factor (CNTF), axokine (a mutein of CNTF), leukemia inhibitory factor (LIF), neutrotrophin 3 (NT-3), neurotrophin-4 (NT-4), nerve growth factor (NGF), insulin-like growth factor II, prostaglandin E2, 30 kD survival factor, taurine, and vitamin A. Other useful cofactors include symptom-alleviating cofactors, including antiseptics, antibiotics, antiviral and antifungal agents and analgesics and anesthetics. Suitable agents for combination treatment with the compounds of the invention include agents known in the art that are able to modulate the activities of complement components.

A combination therapy regimen may be additive, or it may produce synergistic results (e.g., reductions in complement pathway activity more than expected for the combined use of the two agents). In some embodiments, the present invention provide a combination therapy for preventing and/or treating AMD or another complement related ocular disease as described above with a compound of the invention and an anti-angiogenic, such as anti-VEGF agent (including Lucentis and Avastin) or photodynamic therapy (such as verteporfin).

In some embodiments, the present invention provide a combination therapy for preventing and/or treating autoimmune disease as described above with a compound of the invention and a B-Cell or T-Cell modulating agent (for example cyclosporine or analogs thereof, rapamycin, RAD001 or analogs thereof, and the like). In particular, for multiple sclerosis therapy may include the combination of a compound of the invention and a second MS agent selected from fingolimod, cladribine, tysarbi, laquinimod, rebif, avonex and the like.

In one embodiment, the invention provides a method of modulating activity of the complement alternative pathway in a subject, wherein the method comprises administering to the subject a therapeutically effective amount of the compound according to the definition of formula (I). The invention further provides methods of modulating the activity of the complement alternative pathway in a subject by modulating the activity of Factor D, wherein the method comprises administering to the subject a therapeutically effective amount of the compound according to the definition of Formula (I).

In one embodiment, the invention provides a compound according to the definition of formula (I), (la), (VII) or any subformulae thereof, for use as a medicament.

In one embodiment, the invention provides the use of a compound according to the definition of formula (I), (la), (VII) or any subformulae thereof, for the treatment of a disorder or disease in a subject mediated by complement activation. In particular, the invention provides the use of a compound according to the definition of formula (I), (la), (VII) or any subformulae thereof, for the treatment of a disorder or disease mediated by activation of the complement alternative pathway.

In one embodiment, the invention provides the use of a compound according to the definition of formula (I), (la), in the manufacture of a medicament for the treatment of a disorder or disease in a subject characterized by activation of the complement system. More particularly in the manufacture of a medicament for the treatment of a disease or disorder in a subject characterized by over activation of the complement alternative pathway.

In one embodiment, the invention provides the use of a compound according to the definition of formula (I), (la), or subformulae thereof for the treatment of a disorder or disease in a subject characterized by activation of the complement system. More particularly, the invention provides uses of the compounds provided herein in the treatment of a disease or disorder characterized by over activation of the complement alternative pathway or the C3 amplification loop of the alternative pathway. In certain embodiments, the use is in the treatment of a disease or disorder is selected from retinal diseases (such as age-related macular degeneration).

The present invention provides use of the compounds of the invention for treating a disease or disorder associated with increased complement activity by administering to a subject in need thereof an effective amount of the compounds of Formula (I) of the invention. In certain aspects, uses are provided for the treatment of diseases associated with increased activity of the C3 amplification loop of the complement pathway. In certain embodiments, uses of treating or preventing complement mediated diseases are provided in which the complement activation is induced by antibody-antigen interactions, by a component of an autoimmune disease, or by ischemic damage.

In a specific embodiment, the present invention provides use of the compounds of the invention for treating or preventing age-related macular degeneration (AMD). In certain embodiments, patients who are currently asymptomatic but are at risk of developing a symptomatic macular degeneration related disorder are suitable for administration with a compound of the invention. The use in treating or preventing AMD include, but are not limited to, uses in treating or preventing one or more symptoms or aspects of AMD selected from formation of ocular drusen, inflammation of the eye or eye tissue, loss of photoreceptor cells, loss of vision (including loss of visual acuity or visual field), neovascularization (including CNV), retinal detachment, photoreceptor degeneration, RPE degeneration, retinal degeneration, chorioretinal degeneration, cone degeneration, retinal dysfunction, retinal damage in response to light exposure, damage of the Bruch's membrane, and/or loss of RPE function.

The compound of Formula (I) of the invention can be used, inter alia, to prevent the onset of AMD, to prevent the progression of early AMD to advanced forms of AMD including neovascular AMD or geographic atrophy, to slow and/or prevent progression of geographic atrophy, to treat or prevent macular edema from AMD or other conditions (such as diabetic retinopathy, uveitis, or post surgical or non-surgical trauma), to prevent or reduce the loss of vision from AMD, and to improve vision lost due to pre-existing early or advanced AMD. It can also be used in combination with anti-VEGF therapies for the treatment of neovascular AMD patients or for the prevention of neovascular AMD. The present invention further provides methods of treating a complement related disease or disorder by administering to a subject in need thereof an effective amount of the compound(s) of the invention, wherein said disease or disorder is selected from uveitis, adult macular degeneration, diabetic retinopathy, retinitis pigmentosa, macular edema, Behcet's uveitis, multifocal choroiditis, Vogt-Koyangi-Harada syndrome, intermediate uveitis, birdshot retino-chorioditis, sympathetic ophthalmia, ocular dicatricial pemphigoid, ocular pemphigus, nonartertic ischemic optic neuropathy, post-operative inflammation, and retinal vein occlusion.

In some embodiments, the present invention provides uses for treating a complement related disease or disorder. Examples of known complement related diseases or disorders include: neurological disorders, multiple sclerosis, stroke, Guillain Barre Syndrome, traumatic brain injury, Parkinson's disease, disorders of inappropriate or undesirable complement activation, hemodialysis complications, hyperacute allograft rejection, xenograft rejection, interleukin-2 induced toxicity during IL-2 therapy, inflammatory disorders, inflammation of autoimmune diseases, Crohn's disease, adult respiratory distress syndrome, thermal injury including burns or frostbite, myocarditis, post-ischemic reperfusion conditions, myocardial infarction, balloon angioplasty, post-pump syndrome in cardiopulmonary bypass or renal bypass, atherosclerosis, hemodialysis, renal ischemia, mesenteric artery reperfusion after aortic reconstruction, infectious disease or sepsis, immune complex disorders and autoimmune diseases, rheumatoid arthritis, systemic lupus erythematosus (SLE), SLE nephritis, proliferative nephritis, liver fibrosis, hemolytic anemia, myasthenia gravis, tissue regeneration and neural regeneration. In addition, other known complement related disease are lung disease and disorders such as dyspnea, hemoptysis, ARDS, asthma, chronic obstructive pulmonary disease (COPD), emphysema, pulmonary embolisms and infarcts, pneumonia, fibrogenic dust diseases, inert dusts and minerals (e.g., silicon, coal dust, beryllium, and asbestos), pulmonary fibrosis, organic dust diseases, chemical injury (due to irritant gases and chemicals, e.g., chlorine, phosgene, sulfur dioxide, hydrogen sulfide, nitrogen dioxide, ammonia, and hydrochloric acid), smoke injury, thermal injury (e.g., burn, freeze), asthma, allergy, bronchoconstriction, hypersensitivity pneumonitis, parasitic diseases, Goodpasture's Syndrome, pulmonary vasculitis, Pauci-immune vasculitis, immune complex-associated inflammation, uveitis (including Behcet's disease and other sub-types of uveitis), antiphospholipid syndrome.

In a specific embodiment, the present invention provides use of the compounds of the invention for treating a complement related disease or disorder, wherein said disease or disorder is asthma, arthritis (e.g., rheumatoid arthritis), autoimmune heart disease, multiple sclerosis, inflammatory bowel disease, ischemia-reperfusion injuries, Barraquer-Simons Syndrome, hemodialysis, systemic lupus, lupus erythematosus, psoriasis, multiple sclerosis, transplantation, diseases of the central nervous system such as Alzheimer's disease and other neurodegenerative conditions, atypicaly hemolytic uremic syndrome (aHUS), glomerulonephritis (including membrane proliferative glomerulonephritis), blistering cutaneous diseases (including bullous pemphigoid, pemphigus, and epidermolysis bullosa), ocular cicatrical pemphigoid or MPGN II.

In a specific embodiment, the present invention provides use of the compounds of the invention for treating glomerulonephritis. Symptoms of glomerulonephritis include, but not limited to, proteinuria; reduced glomerular filtration rate (GFR); serum electrolyte changes including azotemia (uremia, excessive blood urea nitrogen—BUN) and salt retention, leading to water retention resulting in hypertension and edema; hematuria and abnormal urinary sediments including red cell casts; hypoalbuminemia; hyperlipidemia; and lipiduria. In a specific embodiment, the present invention provides methods of treating paroxysmal nocturnal hemoglobinuria (PNH) by administering to a subject in need thereof an effective amount of a composition comprising an compound of the present invention with or without concomitent administration of a complement C5 inhibitor or C5 convertase inhibitor such as Soliris.

In a specific embodiment, the present invention provides use of the compounds of the invention for reducing the dysfunction of the immune and/or hemostatic systems associated with extracorporeal circulation. The compounds of the present invention can be used in any procedure which involves circulating the patient's blood from a blood vessel of the patient, through a conduit, and back to a blood vessel of the patient, the conduit having a luminal surface comprising a material capable of causing at least one of complement activation, platelet activation, leukocyte activation, or platelet-leukocyte adhesion. Such procedures include, but are not limited to, all forms of ECC, as well as procedures involving the introduction of an artificial or foreign organ, tissue, or vessel into the blood circuit of a patient. More particularly, such procedures include, but are not limited to, transplantation procedures including kidney, liver, lung or heart transplant procedures and islet cell transplant procedures.

The following examples are intended to illustrate the invention and are not to be construed as being limitations thereon. Temperatures are given in degrees centigrade (° C.). If not mentioned otherwise, all evaporations are performed under reduced pressure, typically between about 15 mm Hg and 100 mm Hg (=20-133 mbar). The structure of final products, intermediates and starting materials is confirmed by standard analytical methods, e.g., microanalysis and spectroscopic characteristics, e.g., MS, IR, NMR. Abbreviations used are those conventional in the art.

All starting materials, building blocks, reagents, acids, bases, dehydrating agents, solvents, and catalysts utilized to synthesis the compounds of the present invention are either commercially available or can be produced by organic synthesis methods known to one of ordinary skill in the art (Houben-Weyl 4th Ed. 1952, Methods of Organic Synthesis, Thieme, Volume 21). Further, the compounds of the present invention can be produced by organic synthesis methods known to one of ordinary skill in the art as shown in the following examples.

### ABBREVIATIONS

Ac acetyl
AcCN acetonitrile
ACN acetonitrile
AcOH acetic acid
AIBN azobisisobutyronitrile
app apparent
aq. aqueous
atm atmosphere
Bis(pinacolato)diboron 4,4,4′,4′,5,5,5′,5′-Octamethyl-2,2′-bi-1,3,2-dioxaborolane
Boc tertiary butyl carboxy
Boc-anhydride di-tert-butyl dicarbonate
(Boc)₂O di-tert-butyl dicarbonate
br. broad
BrettPhos palladacycle chloro[2-(dicyclohexylphosphino)-3,6-dimethoxy-2′,4′,6′-tri-i-propyl-1,1′-biphenyl][2-(2-aminoethyl)phenyl]palladium(II)
BSA bovine serum albumin
BuOH butanol
calcd. calculated
CHAPS 3-[(3-cholamidopropyl)dimethylammonio]-1-propanesulfonate
CH₃CN acetonitrile
Cs₂CO₃ cesium carbonate
CoCl₂.6H₂O cobalt dichloride hexahydrate
CVF Cobra Venom Factor
Cy5 2-((1E,3E,5E)-5-(1-(5-carboxypentyl)-3,3-dimethyl-5-sulfoindolin-2-ylidene)penta-1,3-dien-1-yl)-1-ethyl-3,3-dimethyl-5-sulfo-3H-indol-1-ium potassium salt
d doublet
DBU 1,8-diazabicyclo[5.4.0]undec-7-ene
dd doublet of doublets
DCC N,N′-Dicyclohexylcarbodiimide
DCM dichloromethane
DEA diethylamine
DEAD diethyl azodicarboxylate
DIAD diisopropyl azodicarboxylate
DIEA N,N-diisopropylethylamine
DIBAL-H diisobutylaluminium hydride
DIPEA N,N-diisopropylethylamine
DMAP 4,4-dimethylaminopyridine
DME 1,4-dimethoxyethane
DMF N,N-dimethylformamide
Dess-Martin Periodinane Dess-Martin reagent; 1,1,1-Triacetoxy-1,1-dihydro-1,2-benziodoxol-3(1H)-one
DMSO dimethylsulfoxide
ESI electrospray ionization
EtOAc, AcOEt ethyl acetate
Et ethyl
EtOH ethanol
FCC flash column chromatography
g grams
h hour(s)
HATU 2-(1H-7-azabenzotriazol-1-yl)-1,1,3,3-tetramethyl uronium hexafluorophosphate methanaminium
HBTU O-benzotriazole-N,N,N′,N′-tetramethyl-uronium-hexafluoro-phosphate
HC HPLC condition
HEPES 4-(2-hydroxyethyl)-1-piperazineethanesulfonic acid
HFIP 1,1,1,3,3,3-hexafluoro-2-propanol
HPLC high performance liquid chromatography
IPA 2-propanol
IR infrared spectroscopy
L liter(s)
LDA lithium diisopropyl amide
TMP 2,2′,6,6′-tetramethylpiperidine, 2,2′,6,6′-tetramethylpiperidyl
M molar
MHz mega Hertz
m multiplet
Me methyl
MeCN acetonitrile
Mel iodomethane
MeOH methanol
mg milligram(s)
mm millimeter(s)
min minutes
ml milliliter(s)
mL milliliter(s)
mmol millimoles
MP melting point
MS mass spectrometry
Ms₂O methanesulfonyl anhydride
m/z mass to charge ratio
N normal
NaBH₄ sodium borohydride
Na(AcO)₃BH sodium triacetoxyborohydride
NBS—N-Bromo succinamide
NH₄Cl ammonium chloride
NMR nuclear magnetic resonance
PBS phosphate buffered saline
Pd/C palladium on carbon
Pd(dppf)₂Cl₂ CH₂Cl₂ adduct 1,1′-bis(diphenylphosphino)ferrocene-palladium(II)dichloride dichloromethane complex
Pd(dba)₂ bis(dibenzylideneacetone)palladium(0)
Pd₂(dba)₃ tris(dibenzylideneacetone)dipalladium(0)
Pd(PPh₃)₄ tetrakis(triphenylphosphine)palladium(0)
Ph phenyl
ppm parts per million
PyBOP benzotriazol-1-yloxytripyrrolidinophosphonium hexafluorophosphate
Q-Phos 1,2,3,4,5-Pentaphenyl-1′-(di-tert-butylphosphino)ferrocene
rac racemic
RockPhos 2-(di-t-butylphosphino)-3-methoxy-6-methyl-2′,4′,6′-tri-i-propyl-1,1′-biphenyl
RP—reverse phase
rt room temperature
RuPhos 2-dicyclohexylphosphino-2′,6′-diisopropoxybiphenyl
RuPhos palladacycle chloro(2-dicyclohexylphosphino-2′,6′-di-i-propoxy-1,1′-biphenyl)[2-(2-aminoethylphenyl)]palladium(II)
s singlet
sat. saturated
Selectfluor 1-chloromethyl-4-fluoro-1,4-diazoniabicyclo[2.2.2]octane bis(tetrafluoroborate), N-chloromethyl-N′-fluorotriethylenediammonium bis(tetrafluoroborate)
SEM 2-(trimethylsilyl)ethoxymethyl
SEMCI 2-(trimethylsilyl)ethoxymethyl chloride
SFC Supercritical Fluid Chromatography
Sphos palladacycle Chloro(2-dicyclohexylphosphino-2′,6′-dimethoxy-1,1′-biphenyl)[2-(2-aminoethylphenyl)]palladium(II)-methyl-t-butyl ether adduct
t triplet
tᵣ retention time
TBAF tetra-n-butylammonium fluoride
TBAT tetrabutylammonium difluorotriphenylsilicate
TBSCl tert-butyldimethylsilyl chloride
TEA, Et₃N triethylamine
tert- tertiary
tert-butyl Xphos 2-di-tert-butylphosphino-2′,4′,6′-triisopropylbiphenyl
TFA trifluoroacetic acid
THF tetrahydrofuran
Ti(OiPr)₄ titanium isopropoxide
TLC Thin Layer Chromatography
TMSCF₃ trifluoromethyltrimethylsilane
TOSMIC toluenesulfonylmethyl isocyanide
TMS trimethylsilyl
Ts p-toluenesulfonyl
TsOH p-toluenesulfonic acid
UPLC ultra performance liquid chromatography
v/v volume per volume
w/v weight per volume
w/w weight per weight
X-Phos 2-Dicyclohexylphosphino-2′,4′,6′-triisopropylbiphenyl
X-Phos palladacycle Chloro(2-dicyclohexylphosphino-2′,4′,6′-triisopropyl-1,1′-biphenyl)[2-(2-aminoethyl)phenyl)]palladium(II)

The following examples are intended to illustrate the invention and are not to be construed as being limitations thereon. Unless otherwise stated, one or more tautomeric forms of compounds of the examples described hereinafter may be prepared in situ and/or isolated. All tautomeric forms of compounds of the examples described hereafter should be considered to be disclosed. Temperatures are given in degrees centigrade. If not mentioned otherwise, all evaporations are performed under reduced pressure, preferably between about 15 mm Hg and 100 mm Hg (=20-133 mbar). The structure of final products, intermediates and starting materials is confirmed by standard analytical methods, e.g., spectroscopic characteristics, e.g., MS, IR, NMR. Abbreviations used are those conventional in the art.

All starting materials, building blocks, reagents, acids, bases, dehydrating agents, solvents, and catalysts utilized to synthesis the compounds of the present invention are either commercially available or can be produced by organic synthesis methods known to one of ordinary skill in the art (Houben-Weyl 4th Ed. 1952, Methods of Organic Synthesis, Thieme, Volume 21). Further, the compounds of the present invention can be produced by organic synthesis methods known to one of ordinary skill in the art as shown in the following examples. Ionic exchange cartridge PL-HCO₃ MP-SPE used to retain acidic salts are provided by Stratosphere (500 mg, capacity of 0.9 mmol, catalogue number PL3540-C603SPL) Phase separating cartridge ISOLUTE SPEE are provided by Biotage.

Metal scavenger resin SiliaMetS®Thiol used to catch the residual palladium is provided by Silicycle, (Particle Size: 40-63 μm, loading 1.39 mmol/g, catalogue number R51030B).

All reactions are carried out under nitrogen or argon unless otherwise stated. Optical rotations were measured in MeOH.

Proton NMR (¹H NMR) is conducted in deuterated solvent. In certain compounds disclosed herein, one or more ¹H shifts overlap with residual proteo solvent signals; these signals have not been reported in the experimental provided hereinafter.

Multiple parent ion masses are reported for mass spectroscopy data when the compound of the invention contains one or more bromine atoms. Bromine exists as an approximately 1:1 molar ratio of ⁷⁹Br:⁸¹Br. Thus, a compound with a single bromine atom will exhibit two parent mass ions having a difference of 2 amu.

Following preparation methods were used for preparative HPLC.

Method A:
Stationary phase: Waters SunFire™ Prep C18 OBD™ 5 μm, 30×100 mm
Mobile phase: gradient, water with 0.1% TFA/acetonitrile

Method B:
Stationary phase: Gemini® NX 5μ C18 110A 100×30 mm
Mobile phase: gradient, water with 0.1% (28% ammonium hydroxide)/acetonitrile

### TLC Conditions:

R_{f} values for TLC are measured on 5×10 cm TLC plates, silica gel F₂₅₄, Merck, Darmstadt, Germany.

The following methods were used for analytical HPLC:

Method C: Waters XBridge C18, 2.5 μm, 3×30 mm, 10-98% CH₃CN/H₂O/3 min, 98% CH₃CN/0.5 min, CH₃CN and H₂O containing 0.1% TFA, flow: 1.4 mL/min, temperature 40° C.

Method D: Waters UPLC Acquity; column: Acquity HSS T3, 1.8 μm, 2.1*50 mm at 60° C., Eluent A: water+0.05% HCOOH+3.75 mM ammonium acetate, B: ACN+0.04% HCOOH, Gradient: 5 to 98% B in 1.4 min, Flow: 1.0 mL/min

Method E: Waters Sunfire C18, 2.5 mm, 3*30 mm Eluent A: H2O+0.1% TFA, B: ACN+0.1% TFA, Gradient: 10% to 98% B in 2.5 min.

Absolute stereochemistry and/or optical rotations are provided for the embodiments of the invention where applicable. The invention contemplates all stereochemical forms of the compounds provided herein. Where absolute stereochemistry is provided the assessment was made via X-ray diffraction, and/or chemical correlation, and/or at least one chiral center was from a purchased commercial enantiopure (>15:1 er) starting material. In some instances compounds contain two or more chiral centers. The relative stereochemistry of these compounds was assessed via NMR studies and/or X-ray diffraction. In some instances the relative stereochemistry of a diastereomeric pair was not determined and thus the individual diasteromers are identified by the retention time under delineated HPLC conditions and the monikers “first diastereomer” or “second diastereomer”, or “single diastereomer” when only one isomer is isolated and/or available.

In the case of a racemic samples, including intermediates, enantiomers are separated by chromatography using a chiral stationary phase and are identified/differentiated either by HPLC retention time employing a chiral stationary phase and the monikers “enantiomer-1” or “enantiomer-2”, and/or by a specific “+” or “−” sign referring to the rotation of polarized light when this data is available.

In some instances examples possess an acidic functional group as such during final purification procedures samples may contain an undetermined mixture of the free acid along with potassium and/or lithium salts of the titled compound. Small changes in the amount of salt present may change the observed chemical shift or intensity for some peaks in the ¹H NMR spectra.

Inter Alia the following in vitro tests may be used

### Biological Example 1

### Human Complement Factor D ELISA Assay

Recombinant human factor D (expressed in E. coli and purified using standard methods) at 10 nM concentration is incubated with test compound at various concentrations for 1 hour at room temperature in 0.1 M PBS pH 7.4 containing 7.5 mM MgCl2 and 0.075% (w/v) CHAPS. Cobra venom factor and human complement factor B substrate complex is added to a final concentration of 200 nM. After 1 hour incubation at room temperature, the enzyme reaction was stopped by addition of 0.1 M sodium carbonate buffer pH 9.0 containing 0.15 M NaCl and 40 mM EDTA. The product of the reaction, Ba, was quantified by means of an enzyme-linked-immunosorbent assay. IC₅₀ values are calculated from percentage of inhibition of factor D-activity as a function of test compound concentration.

### Biological Example 2

### Human Complement Factor D TR-FRET Assay

### Biological Example 2.1

### 2-((1E,3E,5E)-5-(1-(6-((((3S,5S)-1-(tert-Butoxycarbonyl)-5-((3-chloro-2-fluorobenzyl)carbamoyl)-3-fluoropyrrolidin-3-yl)methyl)amino)-6-oxohexyl)-3,3-dimethyl-5-sulfoindolin-2-ylidene)penta-1,3-dien-1-yl)-1-ethyl-3,3-dimethyl-5-sulfo-3H-indol-1-ium

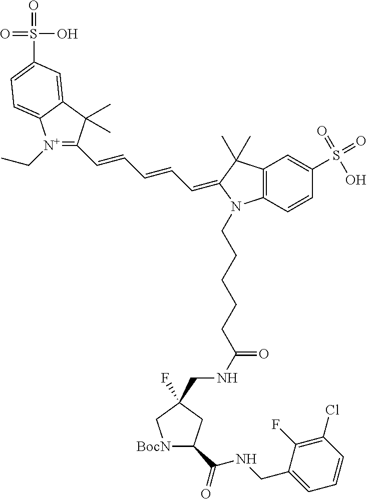 [SMILES: CC[N+]1=C(/C=C/C=C/C=C2/N(CCCCCC(=O)NC[C@@]3(F)C[C@@H](C(=O)NCc4cccc(Cl)c4F)N(C(=O)OC(C)(C)C)C3)c3ccc(S(=O)(=O)O)cc3C2(C)C)C(C)(C)c2cc(S(=O)(=O)O)ccc21]

To a solution of 2-((1E,3E,5E)-5-(1-(5-carboxypentyl)-3,3-dimethyl-5-sulfoindolin-2-ylidene)penta-1,3-dien-1-yl)-1-ethyl-3,3-dimethyl-5-sulfo-3H-indol-1-ium potassium salt (Cy-5, CAS #449175-58-0) (162 mg, 0.233 mmol) in DMF (1 mL) was added HBTU (112 mg, 0.295 mmol) at rt. After 10 min of stirring (2S,4S)-tert-butyl 4-(aminomethyl)-2-((3-chloro-2-fluorobenzyl)carbamoyl)-4-fluoro-pyrrolidine-1-carboxylate (Intermediate B15 step C in WO2012093101) (129 mg, 0.32 mmol) and DIEA (86 μL, 0.491) was added. The blue solution was stirred for 12 h and subsequently purified by preparative HPLC (Waters Sunfire C18 OBD, 5 μm, 30*100 mm, Eluent A: H₂O+0.1% TFA, B: ACN+0.1% TFA, Gradient: 5% to 100% B in 20 min hold 3 min, Flow 40 mL/min) to yield title compound as blue powder. MS (ESI+) m/z 1042.6 (M+).

### Biological Example 2.2

### 2-((1E,3E,5E)-5-(1-(6-((((3S,5S)-1-((1-carbamoyl-1H-indol-3-yl)carbamoyl)-5-((3-chloro-2-fluorobenzyl)carbamoyl)-3-fluoropyrrolidin-3-yl)methyl)amino)-6-oxohexyl)-3,3-dimethyl-5-sulfoindolin-2-ylidene)penta-1,3-dien-1-yl)-1-ethyl-3,3-dimethyl-5-sulfo-3H-indol-1-ium

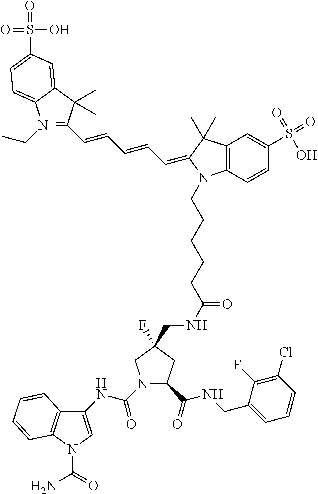 [SMILES: CC[N+]1=C(/C=C/C=C/C=C2/N(CCCCCC(=O)NC[C@@]3(F)C[C@@H](C(=O)NCc4cccc(Cl)c4F)N(C(=O)Nc4cn(C(N)=O)c5ccccc45)C3)c3ccc(S(=O)(=O)O)cc3C2(C)C)C(C)(C)c2cc(S(=O)(=O)O)ccc21]

A solution of 2-((1E,3E,5E)-5-(1-(6-((((3S,5S)-1-(tert-butylcarbamoyl)-5-((3-chloro-2-fluorobenzyl)carbamoyl)-3-fluoropyrrolidin-3-yl)methyl)amino)-6-oxohexyl)-3,3-dimethyl-5-sulfoindolin-2-ylidene)penta-1,3-dien-1-yl)-1-ethyl-3,3-dimethyl-5-sulfo-3H-indol-1-ium (210 mg, 0.2 mmol) in water (2 mL) and TFA (93 μL, 1.2 mmol) was stirred at rt for 12 h. The reaction mixture was concentrated under reduced pressure. At this point TEA (24.8 μL, 0.178 mmol) in THF (1 mL) was added 3-isocyanato-1H-indole-1-carboxamide (CAS #1386456-25-2) (33.5 mg, 0.133 mmol) and 1 ml DMF. The reaction mixture was stirred at rt for 4 h and subsequently purified by preparative HPLC (Waters Sunfire C18 OBD, 5 μm, 30*100 mm, Eluent A: H₂O+0.1% TFA, B: ACN+0.1% TFA, Gradient: 5% to 100% B in 20 min hold 3 min, Flow 40 mL/min), followed by Waters Sunfire C18, 5 μm, 100*19 mm, Eluent A: H₂O+0.1% TFA, B: ACN+0.1% TFA, Gradient: 23% to 53% B, Flow 30 mL/min) to afford title compound. MS (ESI+) m/z 1143.5 (M+), HPLC (Waters Sunfire C18, 2.5 mm, 3*30 mm Eluent A: H₂O+0.1% TFA, B: ACN+0.1% TFA, Gradient: 10% to 98% B in 2.5 min) t_{R}=1.752 min.

### Biological Example 2.3

### Human Complement Factor D TR-FRET Assay

Recombinant human factor D (expressed in E. coli and purified using standard methods) labeled with biotin (10 nM), europium-labeled streptavidin (2 nM) and 2-((1E,3E,5E)-5-(1-(6-((((3S,5S)-1-((1-carbamoyl-1H-indol-3-yl)carbamoyl)-5-((3-chloro-2-fluorobenzyl)carbamoyl)-3-fluoropyrrolidin-3-yl)methyl)amino)-6-oxohexyl)-3,3-dimethyl-5-sulfoindolin-2-ylidene)penta-1,3-dien-1-yl)-1-ethyl-3,3-dimethyl-5-sulfo-3H-indol-1-ium (Biological Example 2.2, 694 nM activity against factor D when tested using the assay of Biological Example 1) (10 nM) were incubated with test compound at various concentrations up to 2 hours at room temperature in 50 mM HEPES buffer, pH 7.4, containing 2.5 mM MgCl₂, 0.01% (w/v) BSA and 0.05% (w/v) CHAPS.

The time-gated decrease in fluorescence intensity related to the competition between labeled and unlabeled factor D ligands was recorded at both 620 nm and 665 nm, 70 μs after excitation at 337 nm using a microplate spectrofluorimeter. IC₅₀ values were calculated from percentage of inhibition of complement factor D-2-((1E,3E,5E)-5-(1-(6-((((3S,5S)-1-((1-carbamoyl-1H-indol-3-yl)carbamoyl)-5-((3-chloro-2-fluorobenzyl)carbamoyl)-3-fluoropyrrolidin-3-yl)methyl)amino)-6-oxohexyl)-3,3-dimethyl-5-sulfoindolin-2-ylidene)penta-1,3-dien-1-yl)-1-ethyl-3,3-dimethyl-5-sulfo-3H-indol-1-ium (Biological Example 2.2, 694 nM activity against factor D when tested using the assay of Biological Example 1) displacement as a function of test compound concentration.

The following Examples, while representing preferred embodiments of the invention, serve to illustrate the invention without limiting its scope.

### Intermediate 1

### Intermediate 1-A

### tert-Butyl 2-(2-bromo-6-nitrophenyl)acetate

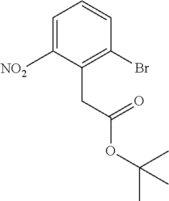 [SMILES: CC(C)(C)OC(=O)Cc1c(Br)cccc1[N+](=O)[O-]]

To a solution of 2-(2-bromo-6-nitrophenyl)acetic acid (CAS #37777-74-5) (2.5 g, 9.6 mmol) and Boc-anhydride (4.20 g, 19.2 mmol) dissolved in t-BuOH (96 ml) was added DMAP (0.352 g, 2.88 mmol), and the reaction was stirred at room temperature for 50 minutes. The reaction was concentrated and purified by flash chromatography (0-50% EtOAc:Heptanes) to provide the title compound. MS (ESI−) m/z 314.2, 316.2 (M−H).

### Intermediate 1-B

### tert-Butyl 2-(2-amino-6-bromophenyl)acetate

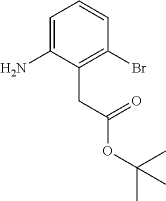 [SMILES: CC(C)(C)OC(=O)Cc1c(N)cccc1Br]

To a round bottom flask containing PtO₂ (0.018 g, 0.080 mmol) under nitrogen atmosphere was added a solution of tert-butyl 2-(2-bromo-6-nitrophenyl)acetate (0.127 g, 0.402 mmol) in EtOH (4.02 ml). This was stirred at room temperature under hydrogen atmosphere for 25 minutes. The reaction was filtered through Celite®, rinsing with MeOH, then concentrated and purified by flash chromatography (0-50% EtOAc:Heptanes) to provide the title compound. MS (ESI+) m/z 286.3, 288.2 (M+H).

### Intermediate 2

### tert-Butyl 2-(2-amino-6-chlorophenyl)acetate

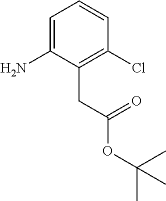 [SMILES: CC(C)(C)OC(=O)Cc1c(N)cccc1Cl]

The title compound was synthesized as described in Intermediate 1 starting with 2-(2-chloro-6-nitrophenyl)acetic acid (CAS #31912-08-0). MS (ESI+) m/z 242.2 (M+H).

### Intermediate 3

### Intermediate 3-A

### tert-Butyl 2-(4-fluoro-2-nitrophenyl)acetate

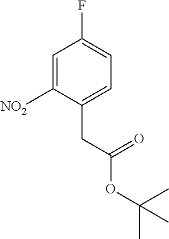 [SMILES: CC(C)(C)OC(=O)Cc1ccc(F)cc1[N+](=O)[O-]]

To a solution of 2-(4-fluoro-2-nitrophenyl)acetic acid (CAS #39616-95-0) (0.15 g, 0.75 mmol) in THF (7.53 ml) was added t-BuOH (0.720 ml, 7.53 mmol) and Boc-anhydride (0.329 g, 1.51 mmol) and DMAP (0.028 g, 0.23 mmol) and this was stirred at room temperature for 20 minutes. This was quenched with water, extracted with EtOAc, dried with MgSO₄, filtered and concentrated. The product was purified by flash chromatography (0-50% EtOAc:Heptanes) to provide the title compound. MS (ESI−) m/z 254.3 (M−H).

### Intermediate 3-B

### Tert-butyl 2-(2-amino-4-fluorophenyl)acetate

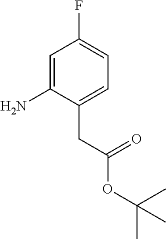 [SMILES: CC(C)(C)OC(=O)Cc1ccc(F)cc1N]

The title compound was synthesized as described in Intermediate 1-B starting with tert-butyl 2-(4-fluoro-2-nitrophenyl)acetate. MS (ESI+) m/z 170.1 (M−tBu+H).

### Intermediate 4

### Intermediate 4-A

### 2-(3-Methyl-2-nitrophenyl)acetic acid

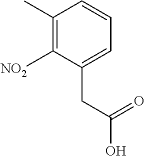

To a solution of potassium tert-butoxide (0.779 g, 6.95 mmol) in dry THF (66.2 ml) under nitrogen at −78° C. was added a solution of 1,3-dimethyl-2-nitrobenzene (CAS #81-20-9) (1.0 g, 6.62 mmol) in THF (5 mL). After 15 minutes 2 pellets of solid CO₂ were added. The reaction was warmed to room temperature and stirred overnight. Saturated aqueous sodium bicarbonate and water and EtOAc were added. The aqueous layer (containing product) was extracted with EtOAc to remove impurities. The aqueous layer was then acidified to pH 1 with conc. HCl, extracted with EtOAc, dried with MgSO₄, filtered and concentrated to give the title compound which was used without further purification. MS (ESI−) m/z 150.1 (M−CO₂H−H).

### Intermediate 4-B

### tert-Butyl 2-(3-methyl-2-nitrophenyl)acetate

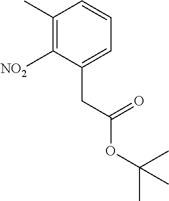 [SMILES: Cc1cccc(CC(=O)OC(C)(C)C)c1[N+](=O)[O-]]

The title compound was synthesized as described in Intermediate 3-A starting with 2-(3-methyl-2-nitrophenyl)acetic acid. MS (ESI−) m/z 250.3 (M−H).

### Intermediate 4-C

### tert-Butyl 2-(2-amino-3-methylphenyl)acetate

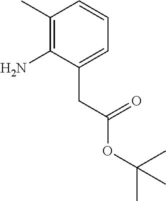 [SMILES: Cc1cccc(CC(=O)OC(C)(C)C)c1N]

The title compound was synthesized as described in Intermediate 1-B starting with tert-butyl 2-(3-methyl-2-nitrophenyl)acetate. MS (ESI+) m/z 222.3 (M+H).

### Intermediate 5

### tert-Butyl 2-(2-amino-3-bromophenyl)acetate

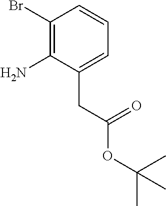 [SMILES: CC(C)(C)OC(=O)Cc1cccc(Br)c1N]

The title compound was synthesized as described in Intermediate 4 starting with 1-bromo-3-methyl-2-nitrobenzene (CAS #52414-97-8). MS (ESI+) m/z 286.1, 288.2 (M+H).

### Intermediate 6

### tert-Butyl 2-(2-amino-5-fluorophenyl)acetate

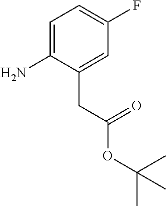 [SMILES: CC(C)(C)OC(=O)Cc1cc(F)ccc1N]

The title compound was synthesized as described in Intermediate 4 starting with 4-fluoro-2-methyl-1-nitrobenzene (CAS #446-33-3). MS (ESI+) m/z 226.2 (M+H).

### Intermediate 7

### tert-Butyl 2-(2-amino-3-methoxyphenyl)acetate

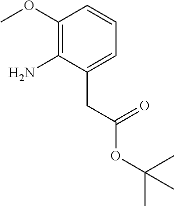 [SMILES: COc1cccc(CC(=O)OC(C)(C)C)c1N]

The title compound was synthesized as described in Intermediate 4 starting with 1-methoxy-3-methyl-2-nitrobenzene (CAS #5345-42-6). MS (ESI+) m/z 238.3 (M+H).

### Intermediate 8

### Intermediate 8-A

### tert-Butyl 2-(3-fluoro-2-nitrophenyl)acetate

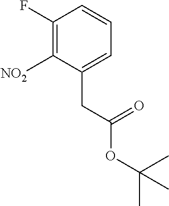 [SMILES: CC(C)(C)OC(=O)Cc1cccc(F)c1[N+](=O)[O-]]

To a solution of 1-bromo-3-fluoro-2-nitrobenzene (CAS #886762-70-5) (110 mg, 0.500 mmol) in THF (2.00 ml) under nitrogen was added Pd(dba)₂ (CAS #32005-36-0) (14.4 mg, 0.025 mmol) and Q-Phos (CAS #312959-24-3) (17.8 mg, 0.025 mmol), then lastly (2-(tert-butoxy)-2-oxoethyl)zinc(II) chloride (CAS #321745-86-2) (0.5M in Ether, 1.10 ml, 0.550 mmol) and the reaction stirred at rt. After 45 minutes the reaction was purified directly by flash chromatography (0-40% EtOAc:Heptanes) to provide the title compound. MS (ESI−) m/z 254.2 (M−H).

### Intermediate 8-B

### tert-Butyl 2-(2-amino-3-fluorophenyl)acetate

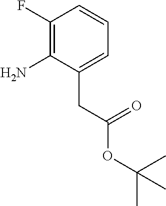 [SMILES: CC(C)(C)OC(=O)Cc1cccc(F)c1N]

The title compound was synthesized as described in Intermediate 1-B starting with tert-butyl 2-(3-fluoro-2-nitrophenyl)acetate. MS (ESI+) m/z 170.1 (M−tBu+H).

### Intermediate 9

### Intermediate 9-A

### tert-Butyl 2-(5-chloro-2-nitrophenyl)acetate

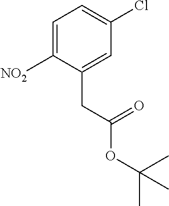 [SMILES: CC(C)(C)OC(=O)Cc1cc(Cl)ccc1[N+](=O)[O-]]

To a solution of 2-bromo-4-chloro-1-nitrobenzene (CAS #63860-31-1) (0.20 g, 0.85 mmol) in THF (3.38 ml) under nitrogen was added Pd(dba)₂ (CAS #32005-36-0) (0.024 g, 0.042 mmol) and Q-Phos (CAS #312959-24-3) (0.030 g, 0.042 mmol), then lastly (2-(tert-butoxy)-2-oxoethyl)zinc(II) chloride (CAS #321745-86-2) (0.5M in Ether, 2.11 ml, 1.06 mmol) and the reaction was stirred at rt. After stirring overnight the reaction was diluted with water and EtOAc, extracted with EtOAc, dried with MgSO₄, filtered and concentrated. The crude product was purified by flash chromatography (0-30% EtOAc:Heptanes) to provide the title compound. MS (ESI−) m/z 270.2 (M−H).

### Intermediate 9-B

### tert-Butyl 2-(2-amino-5-chlorophenyl)acetate

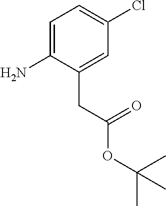 [SMILES: CC(C)(C)OC(=O)Cc1cc(Cl)ccc1N]

The title compound was synthesized as described in Intermediate 1-B starting with tert-butyl 2-(5-chloro-2-nitrophenyl)acetate. MS (ESI+) m/z 186.1 (M−tBu+H).

### Intermediate 10

### tert-Butyl 2-(2-amino-4-methoxyphenyl)acetate

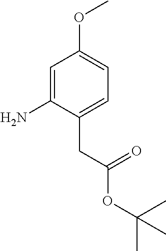 [SMILES: COc1ccc(CC(=O)OC(C)(C)C)c(N)c1]

The title compound was synthesized as described in Intermediate 9 starting with 1-bromo-4-methoxy-2-nitrobenzene (CAS #5344-78-5). MS (ESI+) m/z 182.2 (M−tBu+H).

### Intermediate 11

### tert-Butyl 2-(2-amino-4-methylphenyl)acetate

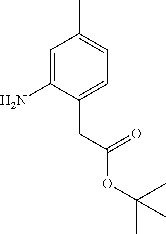 [SMILES: Cc1ccc(CC(=O)OC(C)(C)C)c(N)c1]

The title compound was synthesized as described in Intermediate 9 starting with 1-bromo-4-methyl-2-nitrobenzene (CAS #5326-34-1). MS (ESI+) m/z 166.1 (M−tBu+H).

### Intermediate 12

### tert-Butyl 2-(2-amino-3-chlorophenyl)acetate

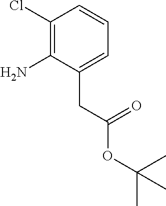 [SMILES: CC(C)(C)OC(=O)Cc1cccc(Cl)c1N]

The title compound was synthesized as described in Intermediate 9 starting with 1-bromo-3-chloro-2-nitrobenzene (CAS #59772-48-4). MS (ESI+) m/z 186.1 (M−tBu+H).

### Intermediate 13

### Intermediate 13-A

### tert-Butyl 2-(2-methyl-6-nitrophenyl)acetate

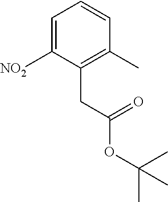 [SMILES: Cc1cccc([N+](=O)[O-])c1CC(=O)OC(C)(C)C]

To a solution of tert-butyl 2-(2-bromo-6-nitrophenyl)acetate (Example 1-A) (0.240 g, 0.759 mmol) in toluene (6.90 ml) under nitrogen was added potassium methyltrifluoroborate (CAS #13862-28-7) (0.463 g, 3.80 mmol) and PdCl₂(dppf).CH₂Cl₂ adduct (CAS #95464-05-4) (0.062 g, 0.076 mmol) and Cs₂CO₃ (0.742 g, 2.28 mmol) and water (0.69 ml), and the reaction was stirred at 100° C. After stirring overnight the reaction was cooled and 1N HCl (4 mL) was added. The reaction was extracted with EtOAc and dried over MgSO₄, filtered and concentrated. The product was purified via flash chromatography (0-50% EtOAc:Heptanes) to provide the title compound. MS (ESI+) m/z 252.3 (M+H).

### Intermediate 13-B

### tert-Butyl 2-(2-amino-3-methylphenyl)acetate

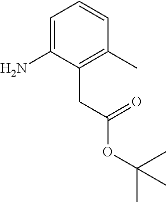 [SMILES: Cc1cccc(N)c1CC(=O)OC(C)(C)C]

The title compound was synthesized as described in Intermediate 1-B starting with tert-butyl 2-(2-methyl-6-nitrophenyl)acetate. MS (ESI+) m/z 222.3 (M+H).

### Intermediate 14

### Intermediate 14-A

### tert-Butyl 2-(2-amino-5-bromophenyl)acetate

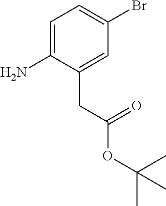 [SMILES: CC(C)(C)OC(=O)Cc1cc(Br)ccc1N]

To a solution of tert-butyl 2-(2-aminophenyl)acetate (CAS #98911-34-3) (100 mg, 0.482 mmol) in DMF (5 mL) at −30° C., NBS (86 mg, 0.482 mmol) was added. After 10 minutes the reaction was quenched with saturated aqueous NaHCO₃ and extracted with EtOAc, dried over Na₂SO₄, filtered and concentrated. The product was purified via flash chromatography (10-50% EtOAc:Heptanes) to provide the title compound. MS (ESI+) m/z 286.2, 288.2 (M+H).

### Intermediate 14-B

### tert-Butyl 2-(2-amino-5-vinylphenyl)acetate H₂N

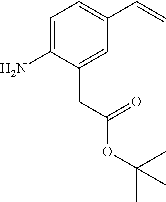 [SMILES: C=Cc1ccc(N)c(CC(=O)OC(C)(C)C)c1]

In a round bottom flask, tert-butyl 2-(2-amino-5-bromophenyl)acetate (0.474 g, 1.66 mmol) was dissolved in DME (12.42 ml) and water (4.14 ml). Vinylboronic anhydride pyridine complex (CAS #442850-89-7) (0.518 g, 2.15 mmol) and Pd(PPh₃)₄ (0.191 g, 0.166 mmol) and K₂CO₃ (0.229 g, 1.66 mmol) were added and the reaction was heated at 110° C. overnight. The reaction was cooled, diluted with water, extracted with EtOAc, dried with MgSO₄, filtered and concentrated. The crude was purified by flash chromatography (0-100% EtOAc:Heptanes) to provide the title compound. MS (ESI+) m/z 178.1 (M−tBu+H).

### Intermediate 14-C

### tert-Butyl 2-(2-amino-5-ethylphenyl)acetate

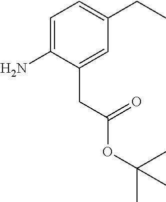 [SMILES: CCc1ccc(N)c(CC(=O)OC(C)(C)C)c1]

The title compound was synthesized as described in Intermediate 1-B (except using EtOAc instead of EtOH as the solvent), starting with tert-butyl 2-(2-amino-5-vinylphenyl)acetate. MS (ESI+) m/z 180.2 (M−tBu+H).

### Intermediate 15

### Intermediate 15-A

### tert-Butyl 2-(4-chloro-2-nitrophenyl)acetate

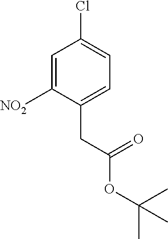 [SMILES: CC(C)(C)OC(=O)Cc1ccc(Cl)cc1[N+](=O)[O-]]

The title compound was synthesized as described in Intermediate 3-A (except using 0.1 eq. of DMAP and 0.9 eq. of Boc-anhydride), starting with 2-(4-chloro-2-nitrophenyl)acetic acid (CAS #37777-71-2). MS (ESI−) m/z 270.1 (M−H).

### Intermediate 15-B

### tert-Butyl 2-(2-nitro-4-vinylphenyl)acetate

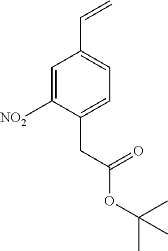 [SMILES: C=Cc1ccc(CC(=O)OC(C)(C)C)c([N+](=O)[O-])c1]

In a microwave vial, to a solution of tert-butyl 2-(4-chloro-2-nitrophenyl)acetate (0.750 g, 2.76 mmol) and vinylboronic anhydride pyridine complex (CAS #442850-89-7) (0.997 g, 4.14 mmol) in THF (18.40 ml) and water (9.20 ml) was added XPhos palladacycle (CAS #1028206-56-5) (0.132 g, 0.276 mmol) and 2M aqueous potassium phosphate (2.76 ml, 5.52 mmol). This solution was placed under nitrogen and heated in the microwave at 120° C. for 1 hr. The reaction mixture was diluted with water, extracted with EtOAc, dried with MgSO₄, filtered and concentrated. The product was purified via flash chromatography (0-50% EtOAc:Heptanes) to provide the title compound. MS (ESI−) m/z 262.1 (M−H).

### Intermediate 15-C

### tert-Butyl 2-(2-amino-4-ethylphenyl)acetate

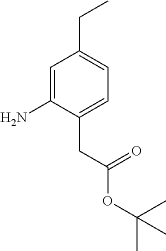 [SMILES: CCc1ccc(CC(=O)OC(C)(C)C)c(N)c1]

The title compound was synthesized as described in Intermediate 1-B (except using EtOAc instead of EtOH as the solvent), starting with tert-butyl 2-(2-nitro-4-vinylphenyl)acetate. MS (ESI+) m/z 236.3 (M+H).

### Intermediate 16

### Intermediate 16-A

### tert-Butyl 2-(2-nitrophenyl)acetate and di-tert-butyl 2-(2-nitrophenyl)malonate

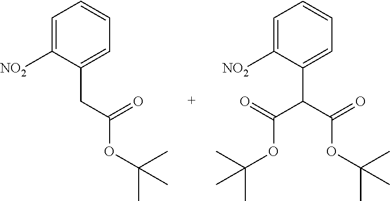 [SMILES: CC(C)(C)OC(=O)Cc1ccccc1[N+](=O)[O-] ; CC(C)(C)OC(=O)C(C(=O)OC(C)(C)C)c1ccccc1[N+](=O)[O-]]

The title compounds (4:1 mixture; inseparable by flash chromatography) were synthesized as described in Intermediate 1-A (the reaction was run at 30° C.), starting with 2-(2-nitrophenyl)acetic acid (CAS #3740-52-1). MS (ESI−) m/z 236.3 and 336.4 (M−H).

### Intermediate 16-B

### tert-Butyl 2-(2-nitrophenyl)acrylate

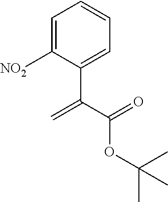 [SMILES: C=C(C(=O)OC(C)(C)C)c1ccccc1[N+](=O)[O-]]

To a solution of a 4:1 mixture of tert-butyl 2-(2-nitrophenyl)acetate and di-tert-butyl 2-(2-nitrophenyl)malonate (2.09 g, 8.81 mmol) in toluene (11.75 ml) was added tetrabutylammonium iodide (0.130 g, 0.352 mmol) and K₂CO₃ (3.65 g, 26.4 mmol) and formaldehyde (37% aqueous solution, 3.28 ml, 44.0 mmol). The reaction was heated at 50° C. for 3 days. The reaction was cooled to room temperature, diluted with water and EtOAc, extracted with EtOAc, dried with MgSO₄, filtered and concentrated. The product was purified by flash chromatography (0-60% DCM:Heptanes) to provide the title compound. ¹H NMR (400 MHz, DMSO-d₆) δ ppm 8.10 (dd, J=8.15, 1.07 Hz, 1H) 7.75-7.84 (m, 1H) 7.59-7.70 (m, 1H) 7.53 (dd, J=7.58, 1.26 Hz, 1H) 6.38 (d, J=0.76 Hz, 1H) 6.01 (d, J=0.76 Hz, 1H) 1.34 (s, 9H).

### Intermediate 16-C

### (±)-tert-Butyl 2-(2-aminophenyl)propanoate

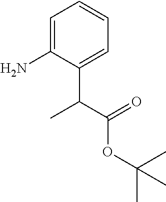 [SMILES: CC(C(=O)OC(C)(C)C)c1ccccc1N]

The title compound was synthesized as described in Intermediate 1-B starting with tert-butyl 2-(2-nitrophenyl)acrylate. MS (ESI+) m/z 166.2 (M−tBu+H).

### Intermediate 17

### Intermediate 17-A. Methyl 2-(4-bromo-2-nitrophenyl)acetate

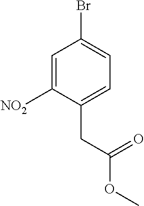

A mixture of 2-(4-bromo-2-nitrophenyl)acetic acid (CAS #6127-11-3) (0.509 g, 1.96 mmol) and HCl (1.25M in MeOH, 15.7 ml, 19.6 mmol) was stirred at room temperature. After 3 days the reaction was concentrated and then purified directly by flash chromatography (0-50% EtOAc:Heptanes) to provide the title compound. MS (ESI−) m/z 272.1, 274.1 (M−H).

### Intermediate 17-B

### Methyl 2-(2-amino-4-bromophenyl)acetate

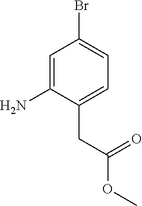

The title compound was synthesized as described in Intermediate 1-B starting with methyl 2-(4-bromo-2-nitrophenyl)acetate. MS (ESI+) m/z 244.1, 246.2 (M+H).

### Intermediate 18

### Intermediate 18-A

### Methyl 2-(4-methyl-2-nitrophenyl)acetate

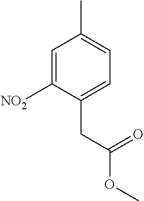

The title compound was synthesized as described in Intermediate 13-A starting with methyl 2-(4-bromo-2-nitrophenyl)acetate (Intermediate 17-A). MS (ESI−) m/z 208.2 (M−H).

### Intermediate 18-B

### Methyl 2-(2-amino-4-methylphenyl)acetate

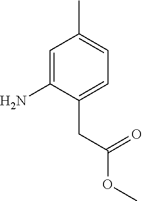

The title compound was synthesized as described in Intermediate 1-B starting with methyl 2-(4-methyl-2-nitrophenyl)acetate. MS (ESI+) m/z 180.3 (M+H).

### Intermediate 19

### Methyl 2-(2-amino-5-bromophenyl)acetate

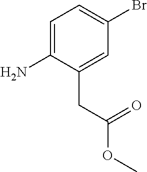

To a solution of methyl 2-(2-aminophenyl)acetate hydrochloride (CAS #49851-36-7) (0.50 g, 2.5 mmol) in DMF (24.8 ml) at 0° C. was added NBS (0.441 g, 2.48 mmol) and this was stirred at 0° C. After 10 minutes the reaction was quenched with saturated aqueous sodium bicarbonate, extracted with EtOAc, washed with water, dried over MgSO₄, filtered and concentrated. This was purified by flash chromatography (0-15% EtOAc:DCM) to provide the title compound. MS (ESI+) m/z 244.1, 246.1 (M+H).

### Intermediate 20

### Intermediate 20-A

### Methyl 2-(4-chloro-2-nitrophenyl)acetate

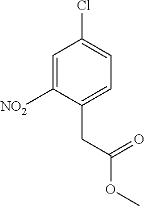

The title compound was synthesized as described in Intermediate 17-A starting with 2-(4-chloro-2-nitrophenyl)acetic acid (CAS #37777-71-2), the reaction was in this case stirred for only 1 day instead of 3 days. MS (ESI−) m/z 228.2 (M−H).

### Intermediate 20-B

### Methyl 2-(2-amino-4-chlorophenyl)acetate

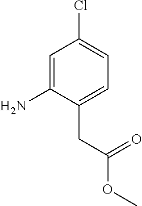

The title compound was synthesized as described in Intermediate 1-B starting with methyl 2-(4-chloro-2-nitrophenyl)acetate. MS (ESI+) m/z 200.2 (M+H).

### Intermediate 21

### tert-Butyl 2-(2-hydroxyphenyl)acetate

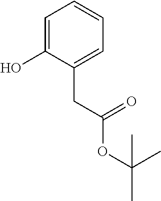

To a suspension of (2-hydroxybenzyl)triphenylphosphonium bromide (CAS #70340-04-4) (50 g, 111 mmol) in DCM (500 mL) was added Et₃N (46.3 mL, 334 mmol) at room temperature. di-tert-Butyl dicarbonate (40.9 mL, 178 mmol) was added and the reaction was stirred at 40° C. for 4 days. The reaction was cooled to room temperature and diluted with DCM and water and the DCM layer was removed, dried and concentrated and absorbed onto silica to purify via FCC (EtOAc-heptane 0-20%) to obtain the title compound. ¹H NMR (400 MHz, DMSO-d₆) δ ppm 9.39 (s, 1H) 6.98-7.11 (m, 2H) 6.66-6.83 (m, 2H) 3.43 (s, 2H) 1.39 (s, 9H).

### Intermediate 22

### Methyl 2-(3-fluoro-2-hydroxyphenyl)acetate

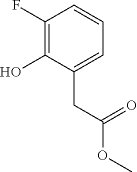

To a solution of 1-bromo-3-fluoro-2-methoxybenzene (CAS #845829-94-9) (2 g, 9.75 mmol) in THF (90 mL) under nitrogen was added Pd(dba)₂ (CAS #32005-36-0) (0.280 g, 0.488 mmol) and Q-Phos (CAS #312959-24-3) (0.347 g, 0.488 mmol), then lastly (2-(tert-butoxy)-2-oxoethyl)zinc(II) chloride (CAS #321745-86-2) (21.46 mL, 10.73 mmol) (0.5M in ether). The reaction was stirred overnight, and then diluted with water and EtOAc, extracted with EtOAc, dried with MgSO₄, filtered and concentrated. The crude was passed through a silica pad eluting with 10% EtOAc in heptane. After evaporation, to a suspension of crude product (1 g, 4.16 mmol) in CH₂Cl₂ (30 mL) at 0° C. was added 1 M BBr₃ in heptane (8.32 mL, 8.32 mmol). After stirring at 0° C. for 10 min, the suspension was allowed to stir at room temperature for another 10 minutes. Methanol (5 mL) was added and the resulting mixture was allowed to stir for 15 minutes. The mixture was partitioned between a saturated aqueous sodium bicarbonate solution and DCM. The organic layer was separated, dried, and concentrated. The residue was purified by silica gel chromatography (0-50% EtOAc-heptane) to provide the title compound. MS (ESI) m/z 185.0 (M+H).

### Intermediate 23

### tert-Butyl 3-bromo-5-fluorobenzylcarbamate

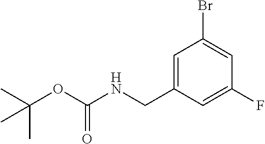 [SMILES: CC(C)(C)OC(=O)NCc1cc(F)cc(Br)c1]

To a solution of (3-bromo-5-fluorophenyl)methanamine (CAS #1094555-68-6) (15 g, 73.5 mmol) in DCM (500 mL), Boc-anhydride (34.1 mL, 147 mmol) was added and the reaction was stirred at room temperature overnight. The reaction was concentrated and purified directly by flash chromatography (50% EtOAc:Heptanes) to provide the title product. MS (ESI−) m/z 246.0, 248.1 (M−tBu-H).

### Intermediate 24

### tert-Butyl 3-fluoro-5-(4,4,5,5-tetramethyl-1,3,2-dioxaborolan-2-yl)benzylcarbamate

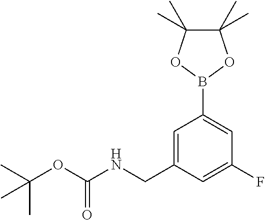 [SMILES: CC(C)(C)OC(=O)NCc1cc(F)cc(B2OC(C)(C)C(C)(C)O2)c1]

To a solution of tert-butyl 3-bromo-5-fluorobenzylcarbamate (Intermediate 23) (11 g, 36 mmol) in DMF (300 mL), bis(pinacolato)diboron (CAS #73183-34-3) (18.4 g, 72.3 mmol), potassium acetate (10.6 g, 108 mmol), PdCl₂(dppf).CH₂Cl₂ adduct (CAS #95464-05-4) (1.32 g, 1.62 mmol) were added and the reaction heated at 110° C. for 5 hours. The reaction was cooled, quenched with water, extracted with EtOAc, dried, filtered and concentrated. The resulting crude product was purified by flash chromatography (20% EtOAc:Heptanes) to provide the title product. MS (ESI+) m/z 296.3 (M−tBu+H).

### Intermediate 25

### (R)-tert-Butyl (1-(3-bromophenyl)ethyl)carbamate

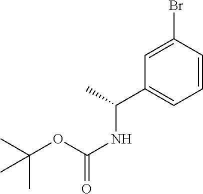 [SMILES: C[C@@H](NC(=O)OC(C)(C)C)c1cccc(Br)c1]

Boc-anhydride (3.71 ml, 16.0 mmol) was added to a solution of (R)-1-(3-bromophenyl)ethanamine (CAS #176707-77-0) (2.0 g, 10.0 mmol) and DIEA (1.83 ml, 10.5 mmol) in DCM (40.0 ml) at room temperature. After stirring overnight the reaction was purified directly by flash chromatography (100% DCM) to provide the title compound. ¹H NMR (400 MHz, DMSO-d₆) δ ppm 7.49 (s, 1H) 7.35-7.46 (m, 2H) 7.20-7.35 (m, 2H) 4.49-4.68 (m, 1H) 1.18-1.49 (m, 12H).

### Intermediate 26

### (R)-tert-Butyl (1-(3-(4,4,5,5-tetramethyl-1,3,2-dioxaborolan-2-yl)phenyl)ethyl)carbamate

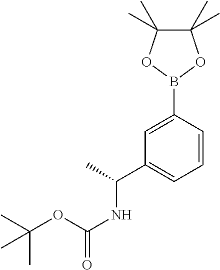 [SMILES: C[C@@H](NC(=O)OC(C)(C)C)c1cccc(B2OC(C)(C)C(C)(C)O2)c1]

The title compound was synthesized as described in Intermediate 24 starting with (R)-tert-butyl (1-(3-bromophenyl)ethyl)carbamate (Intermediate 25). MS (ESI+) m/z 292.0 (M−tBu+H).

### Intermediate 27

### Intermediate 27-A

### (R)-tert-butyl (1-(3-chloro-2-fluorophenyl)ethyl)carbamate

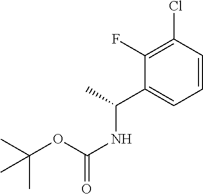 [SMILES: C[C@@H](NC(=O)OC(C)(C)C)c1cccc(Cl)c1F]

Boc-anhydride (0.405 ml, 1.74 mmol) was added to a solution of (R)-1-(3-chloro-2-fluorophenyl)ethanamine hydrochloride (CAS #1253792-97-0) (0.229 g, 1.09 mmol) and DIEA (0.400 ml, 2.29 mmol) in DCM (4.36 ml) at room temperature. After stirring for 2 hours the reaction was purified directly by flash chromatography (0-50% EtOAc:Heptanes) to provide the title compound. ¹H NMR (400 MHz, DMSO-d₆) δ ppm 7.56 (d, J=7.58 Hz, 1H) 7.40-7.50 (m, 1H) 7.29-7.40 (m, 1H) 7.21 (t, J=7.89 Hz, 1H) 4.88 (m, 1H) 1.05-1.48 (m, 12H).

### Intermediate 27-B

### (R)-tert-butyl (1-(2-fluoro-3-(4,4,5,5-tetramethyl-1,3,2-dioxaborolan-2-yl)phenyl)ethyl)carbamate

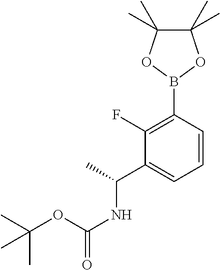 [SMILES: C[C@@H](NC(=O)OC(C)(C)C)c1cccc(B2OC(C)(C)C(C)(C)O2)c1F]

To a solution of (R)-tert-butyl (1-(3-chloro-2-fluorophenyl)ethyl)carbamate (0.162 g, 0.592 mmol) and bis(pinacolato)diboron (0.225 g, 0.888 mmol) and potassium acetate (0.174 g, 1.77 mmol) in dioxane (5.92 ml) under nitrogen was added X-Phos palladacycle (CAS 1028206-56-5) (0.022 g, 0.030 mmol), and the reaction was stirred at 70° C. for 30 minutes, then at 90° C. for 2 hours. The reaction was cooled, diluted with water and EtOAc, extracted with EtOAc, dried over MgSO₄, filtered and concentrated. The crude product was purified by flash chromatography (0-40% EtOAc:Heptanes) to provide the title compound. MS (ESI+) m/z 310.1 (M−tBu+H).

### Intermediate 28

### Intermediate 28-A

### tert-Butyl ((3-bromophenyl)(imino)methyl)carbamate

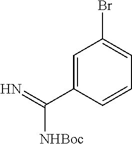 [SMILES: CC(C)(C)OC(=O)NC(=N)c1cccc(Br)c1]

A mixture of 3-bromobenzimidamide hydrochloride (CAS #16796-52-4) (750 mg, 3.18 mmol), Boc₂O (2.29 g, 10.51 mmol) and Et₃N (3.11 ml, 22.29 mmol) in MeOH (20 mL) was heated at 75° C. for 3 hr. The reaction was cooled to rt and concentrated. The residue was purified by silica gel chromatography (EtOAc-heptane 0-50%) to provide the title compound. MS (ESI⁻) m/z 297.1, 299.1 (M−H).

### Intermediate 28-B

### tert-Butyl (imino(3-(4,4,5,5-tetramethyl-1,3,2-dioxaborolan-2-yl)phenyl)methyl)carbamate

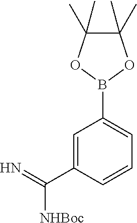 [SMILES: CC(C)(C)OC(=O)NC(=N)c1cccc(B2OC(C)(C)C(C)(C)O2)c1]

A degassed mixture of tert-butyl ((3-bromophenyl)(imino)methyl)carbamate (410 mg, 1.370 mmol), 4,4,4′,4′,5,5,5′,5′-octamethyl-2,2′-bi(1,3,2-dioxaborolane) (CAS #73183-34-3) (522 mg, 2.056 mmol), potassium acetate (404 mg, 4.11 mmol), and PdCl₂(dppf).CH₂Cl₂ adduct (CAS #95464-05-4) (50.1 mg, 0.069 mmol) in DMF (10 mL) was heated at 90° C. overnight. The reaction mixture was partitioned between EtOAc and water. The aqueous layer was extracted with EtOAc and the combined organics were washed with brine, dried (Na₂SO₄) and concentrated. The residue was purified by FCC (EtOAc-heptane 0-50%) to provide the title compound. MS (ESI⁺) m/z 347.2 (M+H).

### Intermediate 29

### Intermediate 29-A

### 1-(3-Bromophenyl)-2-fluoroethanone

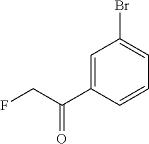 [SMILES: O=C(CF)c1cccc(Br)c1]

Potassium fluoride (2.91 g, 50.0 mmol) was added to the mixture of 2-bromo-1-(3-bromophenyl)ethanone (CAS #18523-22-3) (2.78 g, 10.00 mmol), 1-butyl-3-methylimidazolium tetrafluoroborate (CAS #174501-65-6) (16 mL, 10.00 mmol), and water (900 μL) in CH₃CN (32 mL). The mixture was heated at 60° C. for 2 hr. After cooled to rt, the mixture was extracted with Et₂O. The organic layer was dried (Na₂SO₄) and concentrated. The residue was purified by FCC (EtOAc-heptane 0-20%) to provide the title compound. ¹H NMR (400 MHz, DICHLOROMETHANE-d₂) δ ppm 8.06 (t, J=1.77 Hz, 1H) 7.76-7.87 (m, 2H) 7.44 (t, J=7.89 Hz, 1H) 5.44-5.64 (m, 2H).

### Intermediate 29-B

### (±)-1-(3-Bromophenyl)-2-fluoroethanamine

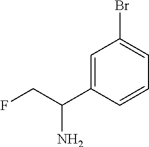 [SMILES: NC(CF)c1cccc(Br)c1]

A mixture of 1-(3-bromophenyl)-2-fluoroethanone (526 mg, 2.424 mmol), Ti(OiPr)₄ (1.435 mL, 4.85 mmol) and 2M ammonia in EtOH solution (12 mL, 24.00 mmol) was stirred at rt overnight. Additional NH₃ in EtOH (6 mL) and Ti(OiPr)₄ (0.7 mL) were added. The mixture was stirred for 6 hr at rt before NaBH₄ (138 mg, 3.64 mmol) was added and the resulting mixture was stirred at rt overnight. Additional NaBH₄ (138 mg) was then added and the mixture was stirred for another 3 hr. 6N HCl was added until the pH reached ˜1-2. The mixture was then partitioned between Et₂O and water. The organic layer was extracted with water. Pellets of NaOH were added to the combined aqueous layers and the pH reached 13. The mixture was then extracted with Et₂O. The combined organics were dried (Na₂SO₄) and concentrated to give an oily residue. 1N HCl (2 mL) was added and resulted in a white suspension. CH₃CN (2 mL) was added and mixture became a homogeneous solution. This was frozen and subjected to lyophilization overnight to provide the title compound as hydrochloride salt. ¹H NMR (HCl salt, 400 MHz, DMSO-d₆) δ ppm 8.99 (d, J=13.77 Hz, 3H) 7.83 (br. s., 1H) 7.52-7.70 (m, 2H) 7.35-7.51 (m, 1H) 4.84 (d, J=4.80 Hz, 1H) 4.65-4.78 (m, 2H).

### Intermediate 29-C

### (S)-tert-Butyl (1-(3-bromophenyl)-2-fluoroethyl)carbamate

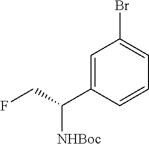 [SMILES: CC(C)(C)OC(=O)N[C@H](CF)c1cccc(Br)c1]

To a solution of (S)-1-(3-bromophenyl)-2-fluoroethanamine (CAS #138462-26-5) (1.0 g, 4.59 mmol) in dichloromethane (23.0 mL) was added di-tert butyl dicarbonate (1.5 g, 6.88 mmol) and triethylamine (1.92 mL). This mixture was stirred for several hours at room temperature followed by concentration in vacuo. The residue was partitioned between EtOAc and a cold 1.0 N HCl aqueous solution; the organic phase was washed with brine, dried over sodium sulfate and concentrated in vacuo. The residue was purified by flash column chromatography on silica gel (heptanes/EtOAc=100:0 to 70:30) to afford the title compound. ¹H NMR (600 MHz, METHANOL-d₄) δ ppm 7.53 (s, 1H) 7.44 (d, J=7.79 Hz, 1H) 7.30-7.34 (m, 1H) 7.24-7.29 (m, 1H) 4.58-4.40 (m, 3H) 1.51 (s, 9H).

### Intermediate 30

### Intermediate 30-A

### (R)-tert-Butyl (1-(3-bromophenyl)-3-hydroxypropyl)carbamate

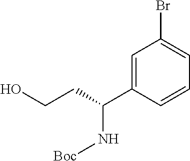 [SMILES: CC(C)(C)OC(=O)N[C@H](CCO)c1cccc(Br)c1]

A mixture of (R)-3-amino-3-(3-bromophenyl)propan-1-ol (CAS #1213827-47-4) (0.66 g, 2.87 mmol), Boc₂O (1 mL, 4.30 mmol) and Et₃N (1.2 mL, 8.60 mmol) in MeOH (10 mL) was stirred at rt for 2 hr. The mixture was concentrated and the residue was partitioned in DCM and Water. The mixture was separated and the aqueous layer was extracted with DCM. The DCM layers were combined and concentrated to provide the title compound. MS (ESI+) m/z 229.9, 231.9 (M-Boc).

### Intermediate 30-B

### (R)-tert-Butyl (3-hydroxy-1-(3-(4,4,5,5-tetramethyl-1,3,2-dioxaborolan-2-yl)phenyl)propyl)carbamate

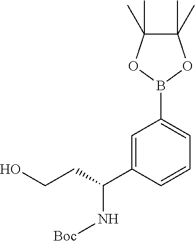 [SMILES: CC(C)(C)OC(=O)N[C@H](CCO)c1cccc(B2OC(C)(C)C(C)(C)O2)c1]

To a solution of (R)-tert-butyl (1-(3-bromophenyl)-3-hydroxypropyl)carbamate (948 mg, 2.87 mmol) in DMF (10 mL), 4,4,4′,4′,5,5,5′,5′-octamethyl-2,2′-bi(1,3,2-dioxaborolane) (CAS #73183-34-3) (1093 mg, 4.31 mmol), potassium acetate (845 mg, 8.61 mmol), PdCl₂(dppf).CH₂Cl₂ adduct (CAS #95464-05-4) (105 mg, 0.144 mmol) were added. The reaction mixture was heated at 110° C. for 2 hr. The reaction mixture was cooled, quenched with water, extracted with EtOAc/Heptane(50%). The organic layers were combined, dried over magnesium sulfate, evaporated and purified using FCC (EtOAc-Heptane 0-50%) to provide the title compound. MS (ESI+) m/z 378.1 (M+H).

### Intermediate 31

### (R)-tert-Butyl (1-(3-(4,4,5,5-tetramethyl-1,3,2-dioxaborolan-2-yl)phenyl)butyl)carbamate

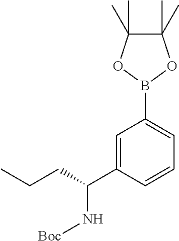 [SMILES: CCC[C@@H](NC(=O)OC(C)(C)C)c1cccc(B2OC(C)(C)C(C)(C)O2)c1]

The title compound was synthesized in a similar manner as described in Intermediate 30 starting from (R)-1-(3-bromophenyl)butan-1-amine hydrochloride (CAS #1391542-02-1) MS (ESI) m/z 376.1 (M+H).

### Intermediate 32

### (R)-tert-Butyl (1-(3-bromo-2-fluorophenyl)butyl)carbamate

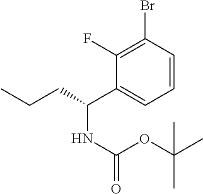 [SMILES: CCC[C@@H](NC(=O)OC(C)(C)C)c1cccc(Br)c1F]

A mixture of (R)-1-(3-bromo-2-fluorophenyl)butan-1-amine hydrochloride (CAS #1213129-43-1) (830 mg, 2.94 mmol), Boc₂O (1.023 ml, 4.41 mmol) and Et₃N (1.228 ml, 8.81 mmol) in MeOH (10 mL) was stirred at rt for 2 hr. The mixture was concentrated and the residue partitioned in DCM and Water. The two layers were separated and the aqueous layer was extracted with DCM. The DCM layers were combined, dried over magnesium sulfate and concentrated to provide the crude product as a white solid. MS (ESI+) m/z 289.9 291.9 (M−^{t}Bu).

### Intermediate 33

### Intermediate 33-A

### 1-(3-Bromo-2-fluorophenyl)-2-fluoroethanone

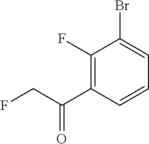 [SMILES: O=C(CF)c1cccc(Br)c1F]

A mixture of 1-(3-bromo-2-fluorophenyl)ethanone (CAS #161957-61-5) (8 g, 36.9 mmol) and Selectfluor (CAS #140681-55-6) (20.89 g, 59.0 mmol) in MeOH (200 mL) was heated at 65° C. for 10 days. The reaction mixture was cooled to rt and filtered. The filtrate was concentrated and the residue was diluted with DCM (150 mL), washed with water and brine, dried (Na₂SO₄) and concentrated. The residue was taken up in DCM (50 mL) and TFA (10 mL) and water (10 mL) were added. The resulting mixture was stirred at 45° C. overnight, and then concentrated in vacuo. The residue was partitioned between EtOAc and sat. NaHCO₃. The aqueous layer was extracted with EtOAc. The combined organics were washed with brine, dried (Na₂SO₄) and concentrated. The residue was purified by FCC (EtOAc-heptane 0-30%) to give the title compound. ¹H NMR (400 MHz, CHLOROFORM-d) δ ppm 7.95 (ddd, J=7.96, 6.19, 1.77 Hz, 1H) 7.81 (ddd, J=7.99, 6.54, 1.77 Hz, 1H) 7.20 (t, J=7.89 Hz, 1H) 5.31-5.59 (m, 2H).

### Intermediate 33-B

### (±)-1-(3-Bromo-2-fluorophenyl)-2-fluoroethanamine

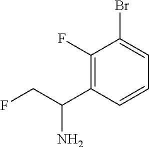 [SMILES: NC(CF)c1cccc(Br)c1F]

A mixture of 1-(3-bromo-2-fluorophenyl)-2-fluoroethanone (5.64 g, 24.00 mmol), Ti(OiPr)₄ (10.66 ml, 36.0 mmol) and 2M ammonia in EtOH (120 ml, 240 mmol) was stirred at rt overnight. Additional 2M NH₃ in EtOH (20 mL) and Ti(OiPr)₄ (4 mL) were added. The mixture was stirred for 8 hr at rt before NaBH₄ (1.362 g, 36.0 mmol) was added and the resulting mixture was stirred at rt overnight. 6N HCl (50 mL) was added portion wise until the pH˜1. Celite was added and mixture was stirred at rt for 2 hr, then filtered. Pellets of NaOH were added to the combined aqueous layers and the pH˜13. The mixture was then extracted with EtOAc (3×). The combined organics were dried (Na₂SO₄) and concentrated to give an oily residue. This material was dissolved in EtOAc and extracted with 1N HCl. The combined aqueous layers were lyophilized to provide the title compound as its HCl salt. ¹H NMR (HCl salt, 400 MHz, DMSO-d₆) δ ppm 8.92 (br. s., 3H) 7.82 (t, J=7.39 Hz, 1H) 7.68 (br. s., 1H) 7.30 (t, J=7.96 Hz, 1H) 4.68-5.02 (m, 3H).

### Intermediate 33-C

### (±)-tert-butyl (1-(3-bromo-2-fluorophenyl)-2-fluoroethyl)carbamate

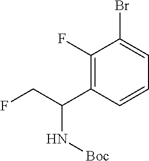 [SMILES: CC(C)(C)OC(=O)NC(CF)c1cccc(Br)c1F]

The title compound was synthesized in a similar manner as described in Intermediate 32 starting with (±)-1-(3-bromo-2-fluorophenyl)-2-fluoroethanamine (Intermediate 33-B). MS (ESI+) m/z 280.0 282.0 (M−tBu).

Resolution of (±)-tert-butyl (1-(3-bromo-2-fluorophenyl)-2-fluoroethyl)carbamate (Intermediate 33-C) was achieved by chiral SFC using a WhelkO1(R,R column) with 5-55% IPA and 5 mM NH₄OH in CO₂ to give the first enantiomer Intermediate 33-C1 (RT=1.35 min, using analytical WhelkO1(S,S column) with 5-55% IPA and 5 mM NH₄OH in CO₂) and the second enantiomer Intermediate 33-C2 (RT=1.07 min, using analytical WhelkO1(S,S column) with 5-55% IPA and 5 mM NH₄OH in CO₂).

### Intermediate 34

### Intermediate 34-A

### (S)-tert-Butyl (1-(3-bromophenyl)-2-hydroxyethyl)carbamate

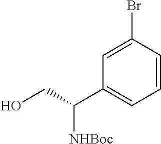 [SMILES: CC(C)(C)OC(=O)N[C@H](CO)c1cccc(Br)c1]

Di-tert-butyl dicarbonate (17.47 mL, 75 mmol) was added in three portions to an ice cold suspension of (S)-2-amino-2-(3-bromophenyl)-ethan-1-ol (CAS #209963-05-3) (10 g, 39.6 mmol) in a mixture of THF (100 mL) and saturated NaHCO₃ (100 mL). The mixture was stirred at room temperature for 18 h and then diluted with EtOAc and water. The aqueous phase was extracted with EtOAc. The combined organic phase was washed with water and brine and then dried over Na₂SO₄. The crude material was purified by flash column chromatography on silica gel (heptane/EtOAc=100:0 to 20:80) to give the title compound. ¹H NMR (400 MHz, DMSO-d₆) δ ppm 7.49 (s, 1H), 7.44-7.40 (m, 1H), 7.31-7.23 (m, 3H), 4.82 (t, J=5.7 Hz, 1H), 4.50 (q, J=6.8 Hz, 1H), 3.52-3.43 (m, 2H), 1.37 (s, 9H).

### Intermediate 34-B

### (S)-tert-Butyl (2-hydroxy-1-(3-(4,4,5,5-tetramethyl-1,3,2-dioxaborolan-2-yl)phenyl)ethyl)carbamate

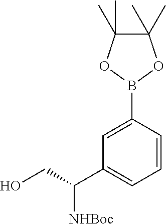 [SMILES: CC(C)(C)OC(=O)N[C@H](CO)c1cccc(B2OC(C)(C)C(C)(C)O2)c1]

To a solution of (S)-tert-butyl (1-(3-bromophenyl)-2-hydroxyethyl)carbamate (5.8 g, 18.34 mmol) and 4,4,4′,4′,5,5,5′,5′-octamethyl-2,2′-bi(1,3,2-dioxaborolane) (CAS #73183-34-3) (9.32 g, 36.7 mmol) in DMF (55.0 mL) was added KOAc (5.4 g, 55.0 mmol); this mixture was degassed for 10 minutes with N₂(gas), and then PdCl₂(dppf).CH₂Cl₂ adduct (CAS #95464-05-4) (0.750 g, 0.917 mmol) was added. The reaction was sealed and heated at 110° C. in an oil bath for 16 hr. The reaction was cooled to room temperature, filtered over a plug of Celite and the filtrate was partitioned between EtOAc/H₂O and the layers separated; the organic phase was washed with brine, combined, dried over Na₂SO₄, and concentrated in vacuo. The residue was purified by flash column chromatography on silica gel (heptanes/EtOAc=100:0 to 50:50) to afford the title compound. ¹H NMR (400 MHz, DMSO-d₆) δ ppm 7.61 (s, 1H) 7.53 (d, J=7.20 Hz, 1H) 7.37-7.42 (m, 1H) 7.29-7.34 (m, 1H) 7.27 (d, J=8.46 Hz, 1H) 4.74 (t, J=5.81 Hz, 1H) 4.49 (d, J=5.68 Hz, 1H) 3.41-3.57 (m, 2H) 1.36 (br. s, 9H), 1.30 (s, 12H).

### Intermediate 35

### Methyl 2-(2-((3-bromo-5-chlorobenzyl)oxy)phenyl)acetate

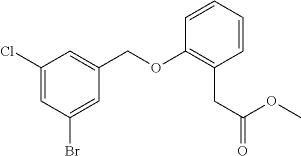 [SMILES: COC(=O)Cc1ccccc1OCc1cc(Cl)cc(Br)c1]

A solution of 3-bromo-5-chloro-benzyl alcohol (CAS #917562-09-5) (5.0 g, 22.58 mmol) and methyl 2-(2-hydroxyphenyl)acetate (CAS #22446-37-3) (4.88 g, 29.3 mmol) and PPh₃ (7.70 g, 29.3 mmol) in THF (100 mL) was cooled to 0° C. in an ice/water bath under nitrogen. DIAD (5.71 ml, 29.3 mmol) was added dropwise. The resulting solution was allowed to warm to room temperature and then stirred overnight. The reaction was quenched with water, extracted with EtOAc, washed with brine, dried with MgSO₄, filtered and concentrated. The crude was purified by flash chromatography (0-40% EtOAc:Heptanes) to provide the title compound.

### Intermediate 36

### (S)-tert-Butyl (1-(3-bromophenyl)-2-methoxyethyl)carbamate

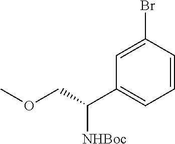 [SMILES: COC[C@@H](NC(=O)OC(C)(C)C)c1cccc(Br)c1]

Mel (1.908 mL, 30.5 mmol) was added to an acetonitrile (10 mL) suspension of (S)-tert-Butyl (1-(3-bromophenyl)-2-hydroxyethyl)carbamate (Intermediate 34-A) (0.965 g, 3.05 mmol) and silver (I) oxide (0.707 g, 3.05 mmol) at room temperature. The resulting suspension was stirred for two days. Another 1.9 mL portion of Mel was added and the reaction was allowed to stir overnight. The mixture was diluted with ethyl acetate, filtered through Celite®, concentrated and purified by FCC (10% ethyl acetate to 80% ethyl acetate in heptane to provide the title compound. MS (ESI−) m/z 272.0, 274.0 (M−^{t}Bu−H).

### Intermediate 37

### (S)-tert-Butyl (1-(3-bromophenyl)-2-ethoxyethyl)carbamate

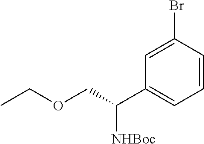 [SMILES: CCOC[C@@H](NC(=O)OC(C)(C)C)c1cccc(Br)c1]

NaH (37.9 mg, 0.949 mmol) was added in one portion to a DMF (5 mL) solution of (S)-tert-butyl (1-(3-bromophenyl)-2-hydroxyethyl)carbamate (Intermediate 34-A) (300 mg, 0.949 mmol) and Etl (0.115 mL, 1.423 mmol) at 0° C. was added. The reaction was allowed to warm to rt and stirred overnight. The reaction was diluted with ethyl acetate and water. The organic layer was separated and washed with brine. The combined organics were dried over sodium sulfate, concentrated and purified using FCC eluting with 100% heptane to 20% ethyl acetate in heptane to provide the title compound. MS (ESI+) m/z 243.9, 245.9 (M−Boc).

### Intermediate 38

### tert-Butyl ((4-chloropyrimidin-2-yl)methyl)carbamate

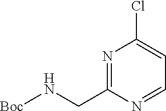 [SMILES: CC(C)(C)OC(=O)NCc1nccc(Cl)n1]

To (4-chloropyrimidin-2-yl)methanamine hydrochloride (CAS #944902-16-3) (0.109 g, 0.605 mmol) in DCM (1.5 mL) was added saturated aq. sodium bicarbonate (1.5 mL). Boc₂O (0.176 mL, 0.757 mmol) was added and the reaction was stirred at room temperature for 2 hours. The reaction was diluted with DCM and passed through a phase separator to obtain the title compound. MS (ESI+) m/z 187.9 (M−tBu+H).

### Intermediate 39

### Intermediate 39-A

### 3-Bromo-2-chlorobenzyl methanesulfonate

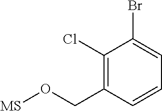

To (3-bromo-2-chlorophenyl)methanol (CAS #1261524-75-7) (0.253 g, 1.142 mmol) in DCM (11.4 mL) at 0° C. was added TEA (0.398 mL, 2.86 mmol) followed by MsCl (0.134 mL, 1.713 mmol) and the ice bath was removed and the solution was allowed to stir at room temperature for 30 minutes. At this point, the reaction was placed in an ice bath and diluted with DCM and a saturated solution of sodium bicarbonate. This mixture was passed through a phase separator to remove the water layer. The organics were dried and concentrated to obtain the title compound. ¹H NMR (400 MHz, DMSO-d₆) δ ppm 7.85 (dd, J=8.07, 1.47 Hz, 1H) 7.63 (dd, J=7.70, 1.47 Hz, 1H) 7.37 (t, J=7.83 Hz, 1H) 5.37 (s, 2H) 3.29 (s, 3H).

### Intermediate 39-B

### (3-Bromo-2-chlorophenyl)methanamine

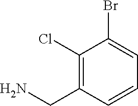

To 3-bromo-2-chlorobenzyl methanesulfonate (0.33 g, 1.102 mmol) was added 7 N ammonia in MeOH (35 mL, 245 mmol) and the reaction was heated at 65° C. for 30 minutes. The reaction was cooled to room temperature and concentrated to obtain the title compound. MS (ESI+) m/z 219.9, 221.8 (M+H).

### Intermediate 40-A

### 3′-(((tert-Butoxycarbonyl)amino)methyl)-5′-fluoro-[1,1′-biphenyl]-3-carboxylic acid

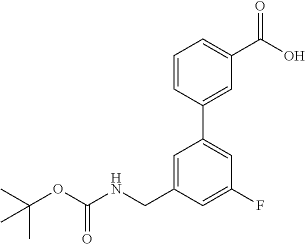 [SMILES: CC(C)(C)OC(=O)NCc1cc(F)cc(-c2cccc(C(=O)O)c2)c1]

Into a round bottom flask was added 3-boronobenzoic acid (1.63 g, 9.83 mmol) and tert-butyl 3-bromo-5-fluorobenzylcarbamate (Intermediate 23) (2.3 g, 7.6 mmol) and DMF (68.1 ml) and H₂O (7.56 ml). Then, 2M aq. K₃PO₄ (18.9 ml, 37.8 mmol) and PdCl₂(dppf).CH₂Cl₂ adduct (CAS #95464-05-4) (0.618 g, 0.756 mmol) were added and the reaction was stirred under nitrogen at 110° C. After 40 minutes this was cooled in an ice bath, diluted with 1N HCl, extracted with EtOAc, dried with MgSO₄, filtered and concentrated. The crude was purified by flash chromatography (0-100% EtOAc:Heptane) to provide the title compound. MS (ESI−) m/z 344.3 (M−H).

### Intermediate 40-B

### 6-(3-(((tert-Butoxycarbonyl)amino)methyl)-5-fluorophenyl)picolinic acid

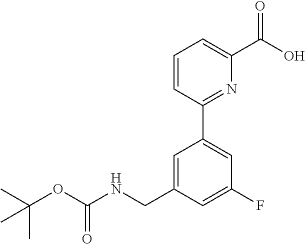 [SMILES: CC(C)(C)OC(=O)NCc1cc(F)cc(-c2cccc(C(=O)O)n2)c1]

In a round bottom flask was added 6-chloropyridine-2-carboxylic acid (CAS #4684-94-0) (1.17 g, 7.40 mmol) and tert-butyl 3-fluoro-5-(4,4,5,5-tetramethyl-1,3,2-dioxaborolan-2-yl)benzylcarbamate (Intermediate 24) (2.0 g, 5.7 mmol) and DMF (51.2 ml) and H₂O (5.7 ml). Then, 2M aq. K₃PO₄ (14.2 ml, 28.5 mmol) and PdCl₂(dppf).CH₂Cl₂ adduct (CAS #95464-05-4) (0.465 g, 0.569 mmol) was added. The reaction was stirred under nitrogen at 110° C. After 1 hour the reaction was diluted with water and EtOAc. This gave three layers: a small aqueous layer at the bottom, an organic layer at the top containing mostly impurities, and a large middle layer containing product and minor impurities. The middle layer was diluted with saturated aqueous NH₄Cl until pH˜8, then extracted with EtOAc, and with a 3:1 DCM:iPrOH mixture. The aqueous layer was then acidified to pH˜1 with conc. HCl, then extracted again with a 3:1 DCM:iPrOH mixture. The organic extractions were combined, dried with MgSO₄, filtered and concentrated. The crude was purified by HPLC (method B) to provide the title compound. MS (ESI−) m/z 345.4 (M−H).

### Intermediate 41

### Methyl 2-(2-(6-chloropicolinamido)phenyl)acetate

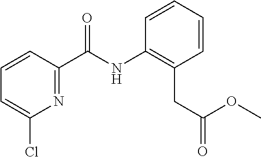 [SMILES: COC(=O)Cc1ccccc1NC(=O)c1cccc(Cl)n1]

In a 40 mL vial, HATU (2.263 g, 5.95 mmol) was added in one portion at room temperature to a DMF solution (10 mL) of methyl 2-(2-aminophenyl)acetate hydrochloride (CAS #49851-36-7) (1 g, 4.96 mmol), 6-chloropyridine-2-carboxylic acid (CAS #4684-94-0) (0.938 g, 5.95 mmol) and DIEA (2.165 mL, 12.40 mmol). The resulting solution was allowed to stir at room temperature (2.5 hr). The reaction was diluted with ethyl acetate, washed with water (twice) and a saturated aq. NaCl solution. The organic phase was dried over sodium sulfate, filtered and concentrated. The residue was purified via FCC (10:1 Heptane/ethyl acetate to 1:1 Heptane/ethyl acetate) to give the title compound. MS (ESI+) m/z 305.3 (M+H).

### Intermediate 42

### Methyl 2-(2-(2-chloroisonicotinamido)phenyl)acetate

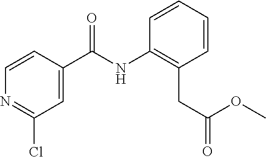 [SMILES: COC(=O)Cc1ccccc1NC(=O)c1ccnc(Cl)c1]

The title compound was prepared from 2-chloroisonicotinic acid (CAS #6313-54-8) using the method described in Intermediate 41. MS (ESI+) m/z 305.2 (M+H).

### Intermediate 43

### Methyl 2-(2-(4-chloropicolinamido)phenyl)acetate

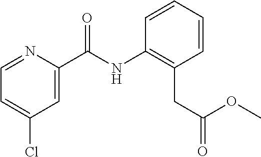 [SMILES: COC(=O)Cc1ccccc1NC(=O)c1cc(Cl)ccn1]

The title compound was prepared from 4-chloropicolinic acid (CAS #5470-22-4) using the method described in Intermediate 41. MS (ESI+) m/z 305.3 (M+H).

### Intermediate 44

### Methyl 2-(2-(5-bromonicotinamido)phenyl)acetate

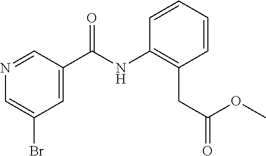 [SMILES: COC(=O)Cc1ccccc1NC(=O)c1cncc(Br)c1]

The title compound was prepared from 5-bromonicotinic acid (CAS #20826-04-4) using the method described in Intermediate 41. MS (ESI+) m/z 349.0, 351.2 (M+H).

### Intermediate 45

### tert-Butyl 2-(2-(3,6-dichloropicolinamido)phenyl)acetate

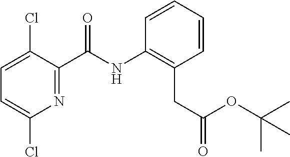 [SMILES: CC(C)(C)OC(=O)Cc1ccccc1NC(=O)c1nc(Cl)ccc1Cl]

The title compound was prepared from 3,6-dichloropicolinic acid (CAS #1702-17-6) using the method described in Intermediate 41. MS (ESI−) m/z 379.0 (M−H).

### Intermediate 46

### Intermediate 46-A

### Ethyl 1-((2-(trimethylsilyl)ethoxy)methyl)-1H-imidazole-2-carboxylate

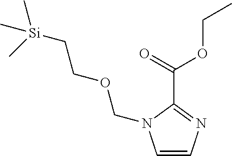 [SMILES: CCOC(=O)c1nccn1COCC[Si](C)(C)C]

SEMCI (1.519 mL, 8.56 mmol) was added to a DMF (10 mL) suspension of K₂CO₃ (1.972 g, 14.27 mmol) and ethyl imidazole-2-carboxylate (CAS #33543-78-1) (1 g, 7.14 mmol). After the mildly exothermic reaction subsided, the mixture was allowed to stir one hour at room temperature and was quenched with the addition of water and ethyl acetate. The organic phase was washed with water, brine, dried (sodium sulfate), filtered and concentrated. Purification of the residue by FCC (100% Heptane to 50% ethyl acetate/Heptane) afforded the title compound. MS (ESI+) m/z 271.4 (M+H).

### Intermediate 46-B

### Ethyl 5-bromo-1-((2-(trimethylsilyl)ethoxy)methyl)-1H-imidazole-2-carboxylate

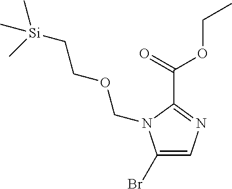 [SMILES: CCOC(=O)c1ncc(Br)n1COCC[Si](C)(C)C]

NBS (1.116 g, 6.27 mmol) was added in one portion to a DCM (5.50 ml)/DMF (5.50 ml) solution of ethyl 1-((2-(trimethylsilyl)ethoxy)methyl)-1H-imidazole-2-carboxylate (1.13 g, 4.18 mmol). The resulting solution was allowed to stir overnight at room temperature. The reaction was partitioned between DCM and a saturated aqueous solution of sodium bicarbonate. The organic phase was washed with water, brine, dried (sodium sulfate), concentrated and then purified by FCC (10% ethyl acetate/heptane to 90% ethyl acetate/heptane) to afford the title compound. ¹H NMR (400 MHz, DMSO-d₆) δ ppm=7.84 (s, 1H), 5.66 (s, 2H), 4.30 (q, J=7.1 Hz, 2H), 3.56-3.50 (m, 2H), 1.30 (t, J=7.1 Hz, 3H), 0.86-0.80 (m, 2H), −0.05-−0.07 (m, 9H).

### Intermediate 46-C

### 5-Bromo-1-((2-(trimethylsilyl)ethoxy)methyl)-I H-imidazole-2-carboxylic acid

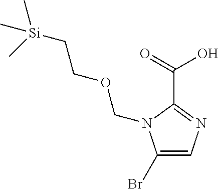 [SMILES: C[Si](C)(C)CCOCn1c(Br)cnc1C(=O)O]

Ethyl 5-bromo-1-((2-(trimethylsilyl)ethoxy)methyl)-1H-imidazole-2-carboxylate (345 mg, 0.988 mmol) in THF (3 mL) and LiOH (1.482 mL, 2.96 mmol, 2M aqueous) was stirred overnight at room temperature. The aqueous phase was lyophilized and the title compounds was used directly in the next step without further purification as its lithium salt. ¹H NMR (400 MHz, DMSO-d₆) δ ppm=7.29 (s, 1H), 5.83 (s, 2H), 3.49 (d, J=16.0 Hz, 2H), 0.84-0.79 (m, J=8.0 Hz, 2H), −0.05 (s, 9H).

### Intermediate 46-D

### tert-Butyl 2-(2-(5-bromo-1-((2-(trimethylsilyl)ethoxy)methyl)-1H-imidazole-2-carboxamido)phenyl)acetate

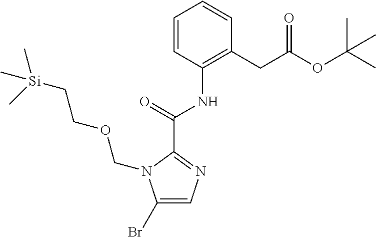 [SMILES: CC(C)(C)OC(=O)Cc1ccccc1NC(=O)c1ncc(Br)n1COCC[Si](C)(C)C]

The title compound was prepared from lithium 5-bromo-1-((2-(trimethylsilyl)ethoxy)methyl)-1H-imidazole-2-carboxylate and tert-butyl 2-(2-aminophenyl)acetate (CAS #98911-34-3) using the method described in Intermediate 41. MS (ESI+) m/z 510.2, 512.3 (M+H).

### Intermediate 47

### Intermediate 47-A

### Ethyl 1-((2-(trimethylsilyl)ethoxy)methyl)-1H-imidazole-5-carboxylate

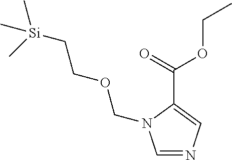 [SMILES: CCOC(=O)c1cncn1COCC[Si](C)(C)C]

The title compound was prepared from ethyl-1H-imidazole-5-carboxylate (CAS #23785-21-9) as described in Intermediate 46-A. MS (ESI+) m/z 271.4 (M+H).

### Intermediate 47-B

### Ethyl 2-bromo-1-((2-(trimethylsilyl)ethoxy)methyl)-1H-imidazole-5-carboxylate

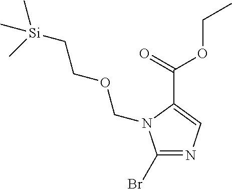 [SMILES: CCOC(=O)c1cnc(Br)n1COCC[Si](C)(C)C]

A carbon tetrachloride (20 mL) suspension of ethyl 1-((2-(trimethylsilyl)ethoxy)methyl)-1H-imidazole-5-carboxylate (733 mg, 2.71 mmol), NBS (511 mg, 2.87 mmol) and AIBN (22.26 mg, 0.136 mmol) were heated at 60° C. for 3 hr. The reaction mixture was diluted with DCM, washed with saturated sodium bicarbonate and brine. The organic phase was dried (sodium sulfate), filtered, concentrated and purified by FCC (10% ethyl acetate in heptane to 50% ethyl acetate in heptane) to afford the title compound. MS (ESI+) m/z 349.1, 351.3 (M+H).

### Intermediate 47-C

### 2-Bromo-1-((2-(trimethylsilyl)ethoxy)methyl)-1H-imidazole-5-carboxylic acid

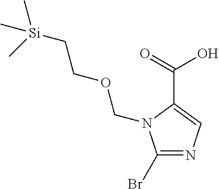 [SMILES: C[Si](C)(C)CCOCn1c(C(=O)O)cnc1Br]

The title compound, isolated as lithium salt, was prepared from ethyl 2-bromo-1-((2-(trimethylsilyl)ethoxy)methyl)-1H-imidazole-5-carboxylate using the method described for Intermediate 46-C. MS (ESI−) m/z 319.2, 321.2 (M−H).

### Intermediate 47-D

### tert-Butyl 2-(2-(2-bromo-1-((2-(trimethylsilyl)ethoxy)methyl)-1H-imidazole-5-carboxamido)phenyl)acetate

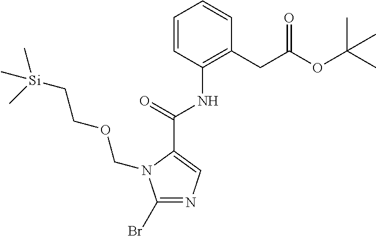 [SMILES: CC(C)(C)OC(=O)Cc1ccccc1NC(=O)c1cnc(Br)n1COCC[Si](C)(C)C]

The title compound was prepared from lithium 2-bromo-1-((2-(trimethylsilyl)ethoxy)methyl)-1H-imidazole-5-carboxylate using the method described in Intermediate 41. MS (ESI+) m/z 510.4, 512.4 (M+H).

### Intermediate 48

### tert-Butyl 2-(2-(3-bromophenylsulfonamido)phenyl)acetate

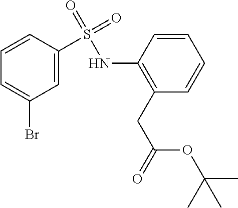 [SMILES: CC(C)(C)OC(=O)Cc1ccccc1NS(=O)(=O)c1cccc(Br)c1]

In a 40 mL vial, 3-bromophenyl sulfonyl chloride (CAS #2905-24-0) (0.75 g, 2.94 mmol) was added in one portion at room temperature to a pyridine (1 mL) solution of tert-butyl 2-(2-aminophenyl)acetate (CAS #98911-34-3) (0.639 g, 3.08 mmol). After 2 hours, ethyl acetate and water were added and the organic phase was washed with water (twice) and brine, dried (sodium sulfate), filtered, concentrated and purified by FCC eluting with ethyl acetate/heptane (gradient 0-100%) to afford the title compound. MS (ESI−) m/z 424.2, 426.3 (M−H).

### Intermediate 49

### Intermediate 49-A

### tert-Butyl 2-(2-(4,6-dichloropicolinamido)phenyl)acetate

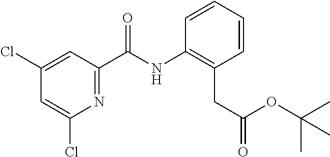 [SMILES: CC(C)(C)OC(=O)Cc1ccccc1NC(=O)c1cc(Cl)cc(Cl)n1]

In a 100 mL round-bottomed flask, HATU (1.834 g, 4.82 mmol) was added in one portion at room temperature to a DMF (20 mL) solution of DIEA (1.149 mL, 6.58 mmol), 4,6-dichloropyridine-2-carboxylic acid (CAS #88912-25-8) (1 g, 4.82 mmol), and tert-butyl 2-(2-aminophenyl)acetate (CAS #98911-34-3) (0.842 g, 4.39 mmol). After 1.5 hr, the reaction was diluted with ethyl acetate, washed with water and brine, dried (sodium sulfate), filtered and concentrated. The residue was purified via FCC (10:1 Hex/EtOAc to 1:10 Hep/EtOAc) to afford the title compound. MS (ESI−) m/z 379.3 (M−H).

### Intermediate 49-B

### tert-Butyl 2-(2-(6-(3-(((tert-butoxycarbonyl)amino)methyl)phenyl)-4-chloropicolinamido)phenyl)acetate

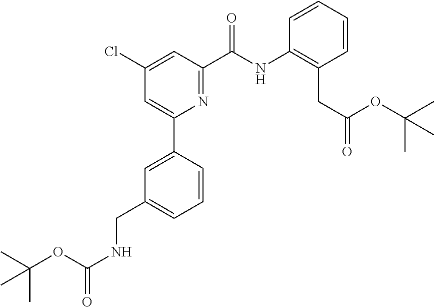 [SMILES: CC(C)(C)OC(=O)Cc1ccccc1NC(=O)c1cc(Cl)cc(-c2cccc(CNC(=O)OC(C)(C)C)c2)n1]

A 40 mL vial was charged with PdCl₂(PPh₃)₂ (0.046 g, 0.066 mmol), Cs₂CO₃ (0.812 g, 2.492 mmol), and (3-(((tert-butoxycarbonyl)amino)methyl)phenyl)boronic acid (CAS #832114-05-3) (0.461 g, 1.836 mmol) and tert-butyl 2-(2-(4,6-dichloropicolinamido)phenyl)acetate (Intermediate 49-A) (0.5 g, 1.311 mmol). Dioxane (5 mL) and water (1 mL) were added, the vial head space was purged with nitrogen and the vial then sealed. After the suspension was stirred for 5 minutes at room temperature, the reaction mixture was heated at 45° C. for 5 hours. The reaction mixture was partitioned between ethyl acetate and water. The organic phase was washed with water, brine and dried (sodium sulfate), filtered and concentrated. The crude residue was purified by FCC using 20-50% ethyl acetate in heptanes gradient to afford the title compound. MS (ESI+) m/z 552.6 (M+H).

### Intermediate 50

### Methyl 2-(2-(3-(4,4,5,5-tetramethyl-1,3,2-dioxaborolan-2-yl)benzamido)phenyl)acetate

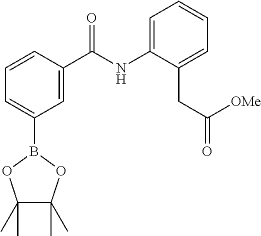 [SMILES: COC(=O)Cc1ccccc1NC(=O)c1cccc(B2OC(C)(C)C(C)(C)O2)c1]

TEA (1.685 mL, 12.09 mmol) was added to a mixture of 3-(4,4,5,5-tetramethyl-1,3,2-dioxaborolan-2-yl)benzoic acid (CAS #269409-73-6) (1 g, 4.03 mmol) and HATU (1.686 g, 4.43 mmol) in DMF (4.5 mL). After 30 min, methyl 2-(2-aminophenyl)acetate hydrochloride (CAS #49851-36-7) (0.666 g, 4.03 mmol) was added and the resulting mixture was stirred at room temperature overnight. The reaction was diluted with a 4:1 EtOAc/heptanes mixture and water. The aqueous phase was extracted with 4:1 EtOAc/heptanes. The combined organic layers were washed with brine, dried over Na₂SO₄, filtered and concentrated. The crude material was purified by flash column chromatography on silica gel (heptane/EtOAc with 10% MeOH, 100:0 to 0:100) to give the title compound. MS (ESI+) m/z 396.4 (M+H).

### Intermediate 51

### Methyl 2-(2-(3-bromo-2-methoxybenzamido)phenyl)acetate

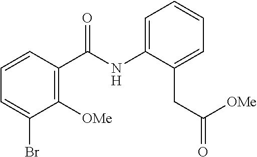 [SMILES: COC(=O)Cc1ccccc1NC(=O)c1cccc(Br)c1OC]

TEA (0.271 mL, 1.948 mmol) was added to a mixture of 3-bromoanisic acid (CAS #101084-39-3) (0.15 g, 0.649 mmol) and HATU (0.272 g, 0.714 mmol) in DMF (1.0 mL). After 70 min, methyl 2-(2-aminophenyl)acetate hydrochloride (CAS #49851-36-7) (0.107 g, 0.649 mmol) was added and the resulting mixture was stirred at room temperature for 18 h. The reaction was diluted with a 4:1 EtOAc/heptanes mixture and water. The aqueous phase was extracted with 4:1 EtOAc/heptanes. The combined organic layers were washed with brine, dried over Na₂SO₄, filtered and concentrated. The crude material was purified by flash column chromatography on silica gel (heptane/EtOAc=100:0 to 0:100) to give the title compound. ¹H NMR (400 MHz, DICHLOROMETHANE-d₂) δ ppm 9.67 (br. s., 1H), 8.03 (dd, J=7.8, 1.8 Hz, 1H), 7.88 (d, J=8.1 Hz, 1H), 7.75 (dd, J=7.8, 1.5 Hz, 1H), 7.37 (t, J=7.7 Hz, 1H), 7.28-7.32 (m, 1H), 7.14-7.24 (m, 2H), 3.97 (s, 2H), 3.74 (s, 6H).

### Intermediate 52

### tert-Butyl 2-(2-((3,5-dibromobenzyl)oxy)phenyl)acetate

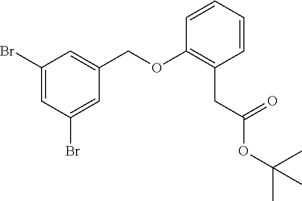 [SMILES: CC(C)(C)OC(=O)Cc1ccccc1OCc1cc(Br)cc(Br)c1]

Tert-butyl 2-(2-hydroxyphenyl)acetate (Intermediate 21) (0.84 g, 4.0 mmol) was dissolved in DMF (20.2 mL) and K₂CO₃ (0.641 g, 4.64 mmol) was added followed by 1,3-dibromo-5-(bromomethyl)benzene (CAS #56908-88-4) (1.46 g, 4.44 mmol). After stirring overnight at room temperature the reaction was diluted with ethyl acetate and water. The organic phase was washed with water, dried with MgSO₄, filtered and concentrated. The reaction was purified by flash chromatography (0-30% EtOAc:Heptanes) to provide the title compound. ¹H NMR (400 MHz, DMSO-d₆) δ ppm 7.79 (t, J=1.77 Hz, 1H) 7.66 (d, J=1.77 Hz, 2H) 7.16-7.29 (m, 2H) 6.98 (d, J=7.58 Hz, 1H) 6.92 (td, J=7.42, 0.95 Hz, 1H) 5.13 (s, 2H) 3.55 (s, 2H) 1.34 (s, 9H).

### Intermediate 53

### tert-Butyl 2-(2-((3-bromo-5-chlorobenzyl)oxy)phenyl)acetate

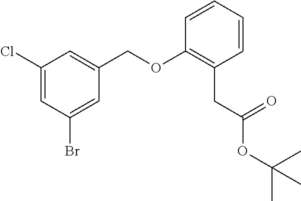 [SMILES: CC(C)(C)OC(=O)Cc1ccccc1OCc1cc(Cl)cc(Br)c1]

The title compound was synthesized as described in Intermediate 52 starting with 1-bromo-3-(bromomethyl)-5-chlorobenzene (CAS #762292-63-7). ¹H NMR (400 MHz, CHLOROFORM-d₂) δ ppm 7.44-7.53 (m, 2H) 7.38 (t, J=1.64 Hz, 1H) 7.17-7.24 (m, 2H) 6.96 (d, J=1.01 Hz, 1H) 6.86 (d, J=8.21 Hz, 1H) 5.02 (s, 2H) 3.60 (s, 2H) 1.42 (s, 9H).

### Intermediate 54

### tert-Butyl 2-(2-((3-bromo-5-cyanobenzyl)oxy)phenyl)acetate

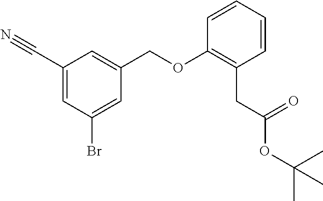 [SMILES: CC(C)(C)OC(=O)Cc1ccccc1OCc1cc(Br)cc(C#N)c1]

The title compound was synthesized as described in Intermediate 52 starting with 3-bromo-5-(bromomethyl)benzonitrile (CAS #124289-24-3). MS (ESI−) m/z 400.1, 402.2 (M−H).

### Intermediate 55

### tert-Butyl 2-(2-((3-bromo-5-(trifluoromethyl)benzyl)oxy)phenyl)acetate

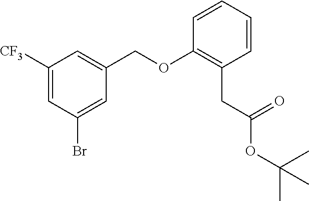 [SMILES: CC(C)(C)OC(=O)Cc1ccccc1OCc1cc(Br)cc(C(F)(F)F)c1]

A solution of (3-bromo-5-(trifluoromethyl)phenyl)methanol (CAS #172023-97-1) (0.525 g, 2.06 mmol) and tert-butyl 2-(2-hydroxyphenyl)acetate (Intermediate 21) (0.557 g, 2.67 mmol) and PPh₃ (0.702 g, 2.67 mmol) in THF (20.6 mL) was cooled to 0° C. in an ice/water bath under nitrogen. DIAD (0.520 mL, 2.67 mmol) was added dropwise. The reaction was allowed to warm to room temperature and then stirred overnight. The reaction was quenched with water, extracted with EtOAc, washed with brine, dried with MgSO₄, filtered and concentrated. This was purified by flash chromatography (0-40% EtOAc:Heptanes) to provide the title compound. ¹H NMR (400 MHz, DMSO-d₆) δ ppm 7.97 (s, 1H) 7.93 (s, 1H) 7.84 (s, 1H) 7.16-7.31 (m, 2H) 7.01 (d, J=7.58 Hz, 1H) 6.88-6.97 (m, 1H) 5.23 (s, 2H) 3.56 (s, 2H) 1.31 (s, 9H).

### Intermediate 56

### tert-Butyl 2-(2-((3-bromo-5-fluorobenzyl)oxy)phenyl)acetate

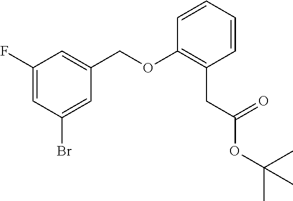 [SMILES: CC(C)(C)OC(=O)Cc1ccccc1OCc1cc(F)cc(Br)c1]

The title compound was synthesized as described in Intermediate 52 starting with 1-bromo-3-(bromomethyl)-5-fluorobenzene (CAS #216755-57-6). ¹H NMR (400 MHz, DMSO-d₆) δ ppm 7.46-7.56 (m, 2H) 7.32 (dd, J=10.61, 1.26 Hz, 1H) 7.16-7.28 (m, 2H) 6.99 (d, J=7.58 Hz, 1H) 6.92 (td, J=7.39, 0.88 Hz, 1H) 5.15 (s, 2H) 3.56 (s, 2H) 1.34 (s, 9H).

### Intermediate 57

### Intermediate 57-A

### Methyl 3-bromo-5-hydroxybenzoate

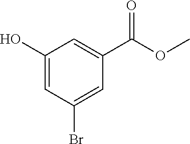

To 3-bromo-5-hydroxybenzoic acid (CAS #140472-69-1) (5.25 g, 24.19 mmol) in MeOH (81 mL) was added conc. HCl (10 mL, 329 mmol) and the reaction was stirred at room temperature for 16 hours and was then heated at 60° C. for 4 hours. The reaction was cooled to room temperature, concentrated and then diluted with water and EA and then the EA layer was separated, dried (sodium sulfate), concentrated and then absorbed onto silica to purify via FCC (0-30% EA:heptanes) to obtain the title compound. MS (ESI−) m/z 229.0, 230.6 (M−H).

### Intermediate 57-B

### Methyl 3-bromo-5-ethoxybenzoate

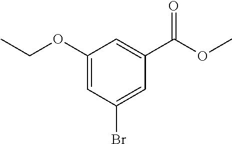

The title compound was synthesized as described in Intermediate 55 starting with methyl 3-bromo-5-hydroxybenzoate and ethanol. ¹H NMR (400 MHz, DMSO-d₆) δ ppm 7.62 (t, J=1.58 Hz, 1H) 7.37-7.47 (m, 2H) 4.11 (q, J=6.95 Hz, 2H) 3.86 (s, 3H) 1.33 (t, J=6.95 Hz, 3H).

### Intermediate 57-C

### (3-Bromo-5-ethoxyphenyl)methanol

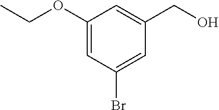

To a solution of methyl 3-bromo-5-ethoxybenzoate (0.458 g, 1.77 mmol) in THF (17.7 mL) under nitrogen was added LiBH₄ (0.128 g, 5.30 mmol) and the reaction was stirred at 50° C. After 90 minutes the reaction was cooled to room temperature and additional LiBH₄ (0.128 g, 5.30 mmol) was added, and then stirred again at 50° C. After overnight the reaction was cooled, diluted with water, sat. brine, and EtOAc. The layers were separated and the aqueous layer was extracted with EtOAc, dried over MgSO₄, filtered and concentrated. The crude product was purified by flash chromatography (0-80% EtOAc:Heptanes) to provide the title compound. ¹H NMR (400 MHz, DMSO-d₆) δ ppm 7.06 (t, J=1.45 Hz, 1H) 6.97 (t, J=2.08 Hz, 1H) 6.86 (dd, J=2.27, 1.26 Hz, 1H) 5.28 (t, J=5.87 Hz, 1H) 4.45 (d, J=5.94 Hz, 2H) 4.03 (q, J=6.95 Hz, 2H) 1.25-1.38 (m, 3H).

### Intermediate 57-D

### tert-Butyl 2-(2-((3-bromo-5-ethoxybenzyl)oxy)phenyl)acetate

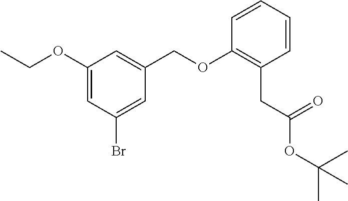 [SMILES: CCOc1cc(Br)cc(COc2ccccc2CC(=O)OC(C)(C)C)c1]

The title compound was synthesized as described in Intermediate 55 starting with (3-bromo-5-ethoxyphenyl)methanol. ¹H NMR (400 MHz, METHANOL-d₄) δ ppm 7.11-7.31 (m, 3H) 6.81-7.07 (m, 4H) 5.05 (s, 2H) 4.04 (q, J=6.99 Hz, 2H) 3.58 (s, 2H) 1.26-1.48 (m, 12H).

### Intermediate 58

### Intermediate 58-A

### Methyl 3-bromo-5-(2,2,2-trifluoroethoxy)benzoate

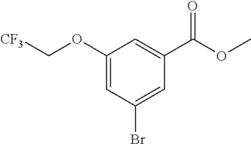 [SMILES: COC(=O)c1cc(Br)cc(OCC(F)(F)F)c1]

To methyl 3-bromo-5-hydroxybenzoate (Intermediate 57-A) (0.311 g, 1.35 mmol) in DMF (13.5 mL) at room temperature under nitrogen was added potassium carbonate (0.372 g, 2.69 mmol) and then 2,2,2-trifluoroethyl trifluoromethanesulfonate (0.469 g, 2.02 mmol). After overnight the reaction was diluted with water and EtOAc, dried with MgSO₄, filtered and concentrated. The product was purified by flash chromatography (0-40% EtOAc:Heptanes) to provide the title compound. ¹H NMR (400 MHz, DMSO-d₆) δ ppm 7.74 (t, J=1.52 Hz, 1H) 7.65 (dd, J=2.40, 1.77 Hz, 1H) 7.56 (dd, J=2.46, 1.33 Hz, 1H) 4.92 (q, J=8.84 Hz, 2H) 3.87 (s, 3H).

### Intermediate 58-B

### (3-Bromo-5-(2,2,2-trifluoroethoxy)phenyl)methanol

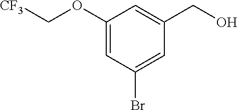 [SMILES: OCc1cc(Br)cc(OCC(F)(F)F)c1]

The title compound was synthesized as described in Intermediate 57-C starting with methyl 3-bromo-5-(2,2,2-trifluoroethoxy)benzoate. ¹H NMR (400 MHz, DMSO-d₆) δ ppm 7.11-7.24 (m, 2H) 6.92-7.05 (m, 1H) 5.34 (t, J=5.81 Hz, 1H) 4.79 (q, J=8.88 Hz, 2H) 4.47 (d, J=5.81 Hz, 2H).

### Intermediate 58-C

### tert-Butyl 2-(2-((3-bromo-5-(2,2,2-trifluoroethoxy)benzyl)oxy)phenyl)acetate

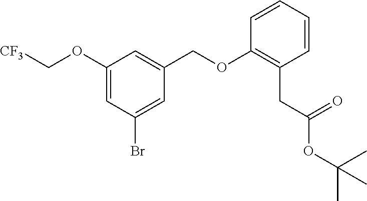 [SMILES: CC(C)(C)OC(=O)Cc1ccccc1OCc1cc(Br)cc(OCC(F)(F)F)c1]

The title compound was synthesized as described in Intermediate 57-D starting with (3-bromo-5-(2,2,2-trifluoroethoxy)phenyl)methanol. ¹H NMR (400 MHz, DMSO-d₆) δ ppm 7.30-7.34 (m, 1H) 7.28 (t, J=2.02 Hz, 1H) 7.18-7.27 (m, 2H) 7.12-7.18 (m, 1H) 6.98 (d, J=7.58 Hz, 1H) 6.91 (td, J=7.42, 0.95 Hz, 1H) 5.10 (s, 2H) 4.82 (q, J=8.84 Hz, 2H) 3.56 (s, 2H) 1.34 (s, 9H).

### Intermediate 59

### Intermediate 59-A

### tert-Butyl 2-(2-((3-bromo-5-(hydroxymethyl)benzyl)oxy)phenyl)acetate

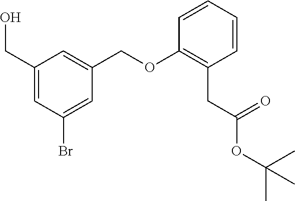 [SMILES: CC(C)(C)OC(=O)Cc1ccccc1OCc1cc(Br)cc(CO)c1]

A solution of (5-bromo-1,3-phenylene)dimethanol (CAS #51760-22-6) (32.5 g, 150 mmol) and tert-butyl 2-(2-hydroxyphenyl)acetate (Intermediate 21) (15.6 g, 74.9 mmol) and PPh₃ (39.3 g, 150 mmol) in THF (250 mL) was cooled to 0° C. in an ice/water bath. DIAD (29.1 mL, 150 mmol) was added dropwise and the resulting yellow solution was allowed to warm to room temperature. After overnight the reaction was quenched with water, extracted with EtOAc, dried with MgSO₄, filtered and concentrated. The crude product was purified by flash chromatography (0-60% EtOAc:Heptanes) to provide the title compound. ¹H NMR (600 MHz, DMSO-d₆) δ ppm 7.50 (s, 1H) 7.46 (s, 1H) 7.36 (s, 1H) 7.16-7.27 (m, 2H) 7.01 (d, J=8.07 Hz, 1H) 6.91 (td, J=7.40, 0.87 Hz, 1H) 5.34 (t, J=5.73 Hz, 1H) 5.11 (s, 2H) 4.50 (d, J=5.69 Hz, 2H) 3.55 (s, 2H) 1.34 (s, 9H).

### Intermediate 59-B

### tert-Butyl 2-(2-((3-bromo-5-formylbenzyl)oxy)phenyl)acetate

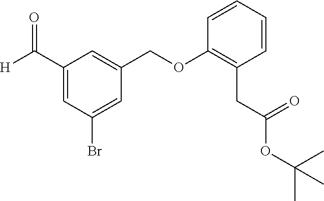 [SMILES: CC(C)(C)OC(=O)Cc1ccccc1OCc1cc(Br)cc(C=O)c1]

To a solution of tert-butyl 2-(2-((3-bromo-5-(hydroxymethyl)benzyl)oxy)phenyl)acetate (28.5 g, 70.0 mmol) in DCM (350 mL) under nitrogen at room temperature, MnO₂ (122 g, 1399 mmol) was added and this was heated at 40° C. After overnight the reaction was filtered through Celite®, concentrated and purified directly by flash chromatography (100% DCM) to provide the title compound. ¹H NMR (400 MHz, DMSO-d₆) δ ppm 9.99 (s, 1H) 8.05 (s, 1H) 7.97 (s, 2H) 7.16-7.30 (m, 2H) 7.03 (d, J=8.08 Hz, 1H) 6.93 (t, J=7.33 Hz, 1H) 5.23 (s, 2H) 3.57 (s, 2H) 1.32 (s, 9H).

### Intermediate 60

### di-tert-Butyl 2,2′-((((5-bromo-1,3-phenylene)bis(methylene))bis(oxy))bis(2,1-phenylene))diacetate

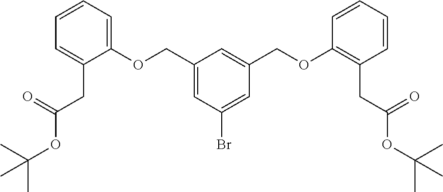 [SMILES: CC(C)(C)OC(=O)Cc1ccccc1OCc1cc(Br)cc(COc2ccccc2CC(=O)OC(C)(C)C)c1]

The title compound was isolated as a minor product from the reaction described in Intermediate 59-A. ¹H NMR (400 MHz, DMSO-d₆) δ ppm 7.60 (s, 2H) 7.51 (s, 1H) 7.15-7.29 (m, 4H) 7.01 (d, J=8.08 Hz, 2H) 6.91 (t, J=7.26 Hz, 2H) 5.12 (s, 4H) 3.56 (s, 4H) 1.23-1.38 (m, 18H).

### Intermediate 61

### tert-Butyl 2-(2-((3-bromo-5-(methoxymethyl)benzyl)oxy)phenyl)acetate

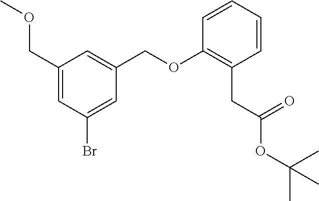 [SMILES: COCc1cc(Br)cc(COc2ccccc2CC(=O)OC(C)(C)C)c1]

To a solution of tert-butyl 2-(2-((3-bromo-5-(hydroxymethyl)benzyl)oxy)phenyl)acetate (Intermediate 59-A) (0.36 g, 0.88 mmol) in THF (8.84 mL) at 0° C. was added NaH (60% in mineral oil, 0.049 g, 1.2 mmol) and then Mel (0.077 mL, 1.2 mmol) and this was stirred at room temperature. After 3 hours additional NaH (0.049 g, 1.2 mmol) and Mel (0.077 mL, 1.2 mmol) were added. After another 90 minutes additional NaH (0.049 g, 1.2 mmol) and Mel (0.077 mL, 1.2 mmol) were added. After overnight the reaction was quenched with saturated aqueous NH₄Cl, extracted with EtOAc, dried with MgSO₄, filtered and concentrated. This was purified by flash chromatography (0-50% EtOAc:Heptanes) to provide the title compound. ¹H NMR (400 MHz, CHLOROFORM-d) δ ppm 7.53 (s, 1H) 7.45 (s, 1H) 7.30 (s, 1H) 7.16-7.25 (m, 2H) 6.94 (td, J=7.45, 1.01 Hz, 1H) 6.87 (d, J=8.08 Hz, 1H) 5.04 (s, 2H) 4.43 (s, 2H) 3.60 (s, 2H) 3.40 (s, 3H) 1.41 (s, 9H).

### Intermediate 62

### (±)-tert-Butyl 2-(2-((3-bromo-5-(1-hydroxyethyl)benzyl)oxy)phenyl)acetate

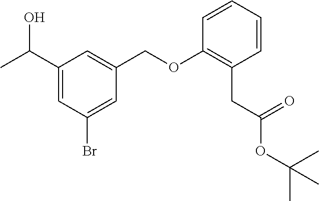 [SMILES: CC(O)c1cc(Br)cc(COc2ccccc2CC(=O)OC(C)(C)C)c1]

To a solution of tert-butyl 2-(2-((3-bromo-5-formylbenzyl)oxy)phenyl)acetate (Intermediate 59-B) (0.40 g, 0.99 mmol) in THF (9.9 mL) at 0° C. under nitrogen, MeMgBr (3.0M in Ether, 0.493 mL, 1.48 mmol) was added and the reaction was warmed to room temperature. After 10 minutes the reaction was quenched with saturated aqueous NH₄Cl, extracted with EtOAc, washed with water, dried with MgSO₄, filtered and concentrated. The crude product was purified by flash chromatography (0-60% EtOAc:Heptanes) to provide the title compound. ¹H NMR (400 MHz, DMSO-d₆) δ ppm 7.48 (s, 2H) 7.38 (s, 1H) 7.16-7.28 (m, 2H) 7.01 (d, J=7.58 Hz, 1H) 6.86-6.95 (m, 1H) 5.31 (d, J=4.40 Hz, 1H) 5.10 (s, 2H) 4.65-4.78 (m, 1H) 3.54 (s, 2H) 1.25-1.38 (m, 12H).

### Intermediate 63

### (±)-tert-Butyl 2-(2-((3-bromo-5-(2,2,2-trifluoro-1-hydroxyethyl)benzyl)oxy)phenyl)acetate

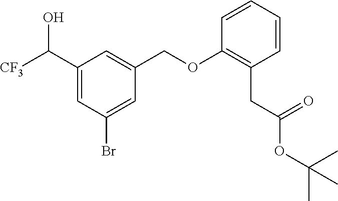 [SMILES: CC(C)(C)OC(=O)Cc1ccccc1OCc1cc(Br)cc(C(O)C(F)(F)F)c1]

To a solution of tert-butyl 2-(2-((3-bromo-5-formylbenzyl)oxy)phenyl)acetate (Intermediate 59-B) (0.404 g, 0.997 mmol) in THF (4.75 mL), trimethyl(trifluoromethyl)silane (0.467 mL, 2.99 mmol) and then TBAF (1M in THF, 2.99 mL, 2.99 mmol) were added at 0° C. under nitrogen. After 10 minutes the reaction mixture was diluted with saturated aqueous NH₄Cl and brine, extracted with EtOAc, dried with MgSO₄, filtered and concentrated. The product was purified by flash chromatography (0-50% EtOAc:Heptanes) to provide the title compound. ¹H NMR (400 MHz, DMSO-d₆) δ ppm 7.67 (s, 1H) 7.63 (s, 1H) 7.57 (s, 1H) 7.16-7.30 (m, 2H) 6.96-7.08 (m, 2H) 6.91 (td, J=7.39, 0.88 Hz, 1H) 5.18-5.28 (m, 1H) 5.14 (s, 2H) 3.55 (s, 2H) 1.26-1.40 (m, 9H).

### Intermediate 64

### (±)-tert-Butyl 2-(2-((3-bromo-5-(cyclohexyl(hydroxy)methyl)benzyl)oxy)phenyl)acetate

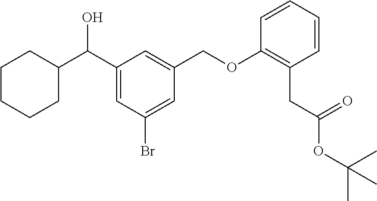 [SMILES: CC(C)(C)OC(=O)Cc1ccccc1OCc1cc(Br)cc(C(O)C2CCCCC2)c1]

To a solution of tert-butyl 2-(2-((3-bromo-5-formylbenzyl)oxy)phenyl)acetate (Intermediate 59-B) (0.40 g, 0.99 mmol) in THF (9.87 mL) at 0° C. under nitrogen, cyclohexylmagnesium chloride (2.0M in Ether, 0.617 mL, 1.23 mmol) was added and this was warmed to room temperature. After 2 hours the reaction was quenched with saturated aqueous NH₄Cl, extracted with EtOAc, washed with water, dried with MgSO₄, filtered and concentrated. This was purified by flash chromatography (0-50% EtOAc:Heptanes) to provide the title compound. ¹H NMR (400 MHz, DMSO-d₆) δ ppm 7.48 (s, 1H) 7.39 (s, 1H) 7.30 (s, 1H) 7.15-7.27 (m, 2H) 7.00 (d, J=7.58 Hz, 1H) 6.90 (td, J=7.39, 0.88 Hz, 1H) 5.22 (d, J=4.55 Hz, 1H) 5.11 (s, 2H) 4.21-4.30 (m, 1H) 3.54 (s, 2H) 1.73 (d, J=11.49 Hz, 4H) 1.39-1.50 (m, 1H) 1.34 (s, 9H) 0.87-1.15 (m, 6H).

### Intermediate 65

### Intermediate 65-A

### Methyl 3-bromo-5-((2-(2-(tert-butoxy)-2-oxoethyl)phenoxy)methyl)benzoate

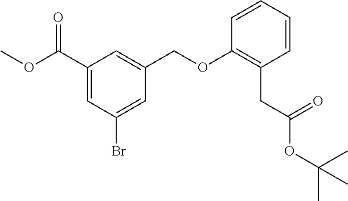 [SMILES: COC(=O)c1cc(Br)cc(COc2ccccc2CC(=O)OC(C)(C)C)c1]

To a suspension of tert-butyl 2-(2-((3-bromo-5-formylbenzyl)oxy)phenyl)acetate (Intermediate 59-B) (0.30 g, 0.74 mmol) in MeOH (7.40 mL) under nitrogen, a solution of KOH (0.125 g, 2.22 mmol) in MeOH (3 mL) was added. This was cooled to 0° C. and a solution of iodine (0.244 g, 0.962 mmol) in MeOH (3 mL) was added. After overnight the reaction was quenched with saturated aqueous sodium thiosulfate, extracted with EtOAc, washed with water, dried with MgSO₄, filtered and concentrated to give the title compound. ¹H NMR (400 MHz, DMSO-d₆) δ ppm 7.97-8.06 (m, 2H) 7.94 (t, J=1.71 Hz, 1H) 7.15-7.31 (m, 2H) 7.02 (d, J=7.58 Hz, 1H) 6.92 (td, J=7.42, 0.95 Hz, 1H) 5.21 (s, 2H) 3.87 (s, 3H) 3.55 (s, 2H) 1.32 (s, 9H).

### Intermediate 65-B

### Tert-butyl 2-(2-((3-bromo-5-(2-hydroxypropan-2-yl)benzyl)oxy)phenyl)acetate

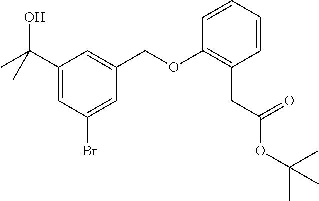 [SMILES: CC(C)(C)OC(=O)Cc1ccccc1OCc1cc(Br)cc(C(C)(C)O)c1]

To a solution of methyl 3-bromo-5-((2-(2-(tert-butoxy)-2-oxoethyl)phenoxy)methyl)benzoate (0.28 g, 0.643 mmol) in THF (6.43 mL) at 0° C. under nitrogen, MeMgBr (3.0M in Ether, 0.643 mL, 1.93 mmol) was added and the reaction was warmed to room temperature. After 30 minutes additional MeMgBr (0.107 mL) was added. After 10 more minutes the reaction was quenched with saturated aqueous NH₄Cl, extracted 2× with EtOAc, washed with water, dried with MgSO₄, filtered and concentrated. The reaction was purified by flash chromatography (0-60% EtOAc:Heptanes) to provide the title compound. ¹H NMR (400 MHz, DMSO-d₆) δ ppm 7.60 (t, J=1.71 Hz, 1H) 7.50 (s, 1H) 7.43-7.49 (m, 1H) 7.15-7.29 (m, 2H) 7.01 (d, J=7.70 Hz, 1H) 6.85-6.94 (m, 1H) 5.18 (s, 1H) 5.10 (s, 2H) 3.54 (s, 2H) 1.42 (s, 6H) 1.32 (s, 9H).

### Intermediate 66

### tert-Butyl 2-(2-((3-bromo-4-methylbenzyl)oxy)phenyl)acetate

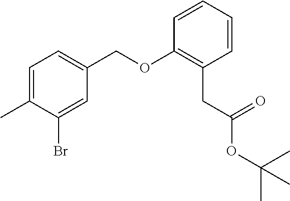 [SMILES: Cc1ccc(COc2ccccc2CC(=O)OC(C)(C)C)cc1Br]

The title compound was synthesized as described in Intermediate 55 starting with (3-bromo-4-methylphenyl)methanol (CAS #68120-35-4). ¹H NMR (400 MHz, CHLOROFORM-d) δ ppm 7.64 (s, 1H) 7.31 (d, J=1.38 Hz, 1H) 7.21-7.28 (m, 3H) 6.97 (td, J=7.43, 0.92 Hz, 1H) 6.92 (d, J=8.07 Hz, 1H) 5.05 (s, 2H) 3.63 (s, 2H) 2.44 (s, 3H) 1.44 (s, 9H).

### Intermediate 67

### tert-Butyl 2-(2-((3-bromo-2-methylbenzyl)oxy)phenyl)acetate

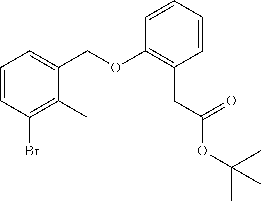 [SMILES: Cc1c(Br)cccc1COc1ccccc1CC(=O)OC(C)(C)C]

The title compound was synthesized as described in Intermediate 55 starting with (3-bromo-2-methylphenyl)methanol (CAS #83647-43-2). ¹H NMR (400 MHz, CHLOROFORM-d) δ ppm 7.54 (d, J=7.96 Hz, 1H) 7.40 (d, J=7.45 Hz, 1H) 7.19-7.28 (m, 2H) 7.06 (t, J=7.83 Hz, 1H) 6.90-6.98 (m, 2H) 5.06 (s, 2H) 3.57 (s, 2H) 2.43 (s, 3H) 1.36 (s, 9H).

### Intermediate 68

### (±)-tert-Butyl 2-(2-((3-bromo-5-(1-methoxyethyl)benzyl)oxy)phenyl)acetate

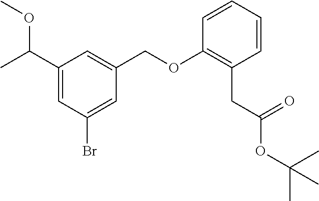 [SMILES: COC(C)c1cc(Br)cc(COc2ccccc2CC(=O)OC(C)(C)C)c1]

The title compound was synthesized as described in Intermediate 61 starting with tert-butyl 2-(2-((3-bromo-5-(1-hydroxyethyl)benzyl)oxy)phenyl)acetate (Intermediate 62). ¹H NMR (400 MHz, DMSO-d₆) δ ppm 7.51-7.61 (m, 1H) 7.41-7.49 (m, 1H) 7.38 (s, 1H) 7.15-7.30 (m, 2H) 7.02 (d, J=7.45 Hz, 1H) 6.91 (td, J=7.39, 1.01 Hz, 1H) 5.04-5.20 (m, 2H) 4.33 (q, J=6.40 Hz, 1H) 3.54 (s, 2H) 3.14 (s, 3H) 1.24-1.40 (m, 12H).

### Intermediate 69

### Methyl 2-(2-((3-bromo-4-fluorobenzyl)oxy)phenyl)acetate

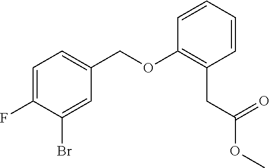 [SMILES: COC(=O)Cc1ccccc1OCc1ccc(F)c(Br)c1]

The title compound was synthesized as described in Intermediate 55 starting with (3-bromo-4-fluorophenyl)methanol (CAS #77771-03-0) and methyl 2-(2-hydroxyphenyl)acetate (CAS #22446-37-3). ¹H NMR (400 MHz, CHLOROFORM-d) δ ppm 7.63 (dd, J=6.57, 2.15 Hz, 1H) 7.29-7.35 (m, 1H) 7.20-7.28 (m, 2H) 7.13 (t, J=8.40 Hz, 1H) 6.96 (td, J=7.45, 1.01 Hz, 1H) 6.86-6.90 (m, 1H) 5.02 (s, 2H) 3.69 (s, 2H), 3.67 (s, 3H).

### Intermediate 70

### Methyl 2-(2-((3-bromo-2-fluorobenzyl)oxy)phenyl)acetate

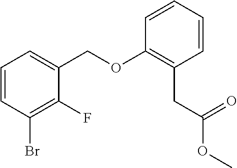 [SMILES: COC(=O)Cc1ccccc1OCc1cccc(Br)c1F]

The title compound was synthesized as described in Intermediate 55 starting with (3-bromo-2-fluorophenyl)methanol (CAS #261723-32-4) and methyl 2-(2-hydroxyphenyl)acetate (CAS #22446-37-3). ¹H NMR (400 MHz, CHLOROFORM-d) δ ppm 7.51 (td, J=7.29, 1.58 Hz, 1H) 7.42-7.48 (m, 1H) 7.20-7.29 (m, 2H) 7.03-7.09 (m, 1H) 6.90-6.99 (m, 2H) 5.17 (s, 2H) 3.68 (s, 2H), 3.65 (s, 3H).

### Intermediate 71

### Methyl 2-(2-((7-bromobenzo[d][1,3]dioxol-5-yl)methoxy)phenyl)acetate

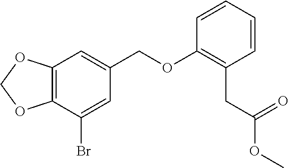 [SMILES: COC(=O)Cc1ccccc1OCc1cc(Br)c2c(c1)OCO2]

The title compound was synthesized as described in Intermediate 52 starting with 4-bromo-6-(bromomethyl)benzo[d][1,3]dioxole (CAS #859968-65-3) and methyl 2-(2-hydroxyphenyl)acetate (CAS #22446-37-3). ¹H NMR (400 MHz, CHLOROFORM-d) δ ppm 7.10-7.23 (m, 2H) 6.93-6.99 (m, 1H) 6.87 (td, J=7.45, 1.01 Hz, 1H) 6.80 (d, J=8.21 Hz, 1H) 6.76 (d, J=0.88 Hz, 1H) 5.97 (s, 2H) 4.88 (s, 2H) 3.61 (br s., 3H) 3.59 (s, 2H).

### Intermediate 72

### Methyl 2-(2-((3-bromo-4-methoxybenzyl)oxy)phenyl)acetate

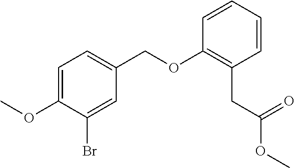

The title compound was synthesized as described in Intermediate 55 starting with (3-bromo-4-methoxyphenyl)methanol (CAS #38493-59-3) and methyl 2-(2-hydroxyphenyl)acetate (CAS #22446-37-3). ¹H NMR (400 MHz, CHLOROFORM-d) δ ppm 7.61 (d, J=2.02 Hz, 1H) 7.31 (dd, J=8.46, 2.15 Hz, 1H) 7.17-7.26 (m, 2H) 6.84-7.00 (m, 3H) 4.99 (s, 2H) 3.91 (s, 2H) 3.67 (s, 3H).

### Intermediate 73

### Intermediate 73-A

### (R)-Methyl 3-bromo-5-((tetrahydrofuran-2-yl)methoxy)benzoate

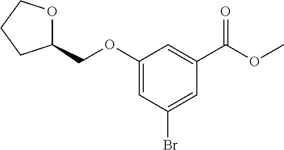 [SMILES: COC(=O)c1cc(Br)cc(OC[C@H]2CCCO2)c1]

The title compound was synthesized as described in Intermediate 55 starting with methyl 3-bromo-5-hydroxybenzoate (Intermediate 57-A) and (R)-(tetrahydrofuran-2-yl)methanol (CAS #22415-59-4). MS (ESI+) m/z 314.8, 316.8 (M+H).

### Intermediate 73-B

### (R)-(3-Bromo-5-((tetrahydrofuran-2-yl)methoxy)phenyl)methanol

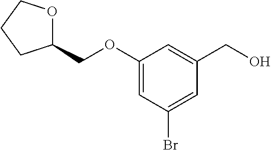 [SMILES: OCc1cc(Br)cc(OC[C@H]2CCCO2)c1]

To (R)-methyl 3-bromo-5-((tetrahydrofuran-2-yl)methoxy)benzoate (0.127 g, 0.403 mmol) in THF (4 mL) at 0° C. was added 1M LiAlH₄ in THF (0.806 mL, 0.806 mmol) and stirred for 1 hour. The reaction was quenched with saturated aq. solution of ammonium chloride at 0° C., diluted with EA and a saturated solution of sodium potassium tartrate. The layers were separated and the organic layer was removed, dried and concentrated to an oil that was absorbed onto silica and purified via FCC (0-100% EA:heptanes) to obtain the title compound. MS (ESI+) m/z 286.7, 288.7 (M+H).

### Intermediate 73-C

### (R)-Methyl 2-(2-((3-bromo-5-((tetrahydrofuran-2-yl)methoxy)benzyl)oxy)phenyl)acetate

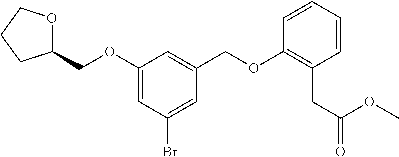 [SMILES: COC(=O)Cc1ccccc1OCc1cc(Br)cc(OC[C@H]2CCCO2)c1]

To (R)-(3-bromo-5-((tetrahydrofuran-2-yl)methoxy)phenyl)methanol (88 mg, 0.306 mmol) in THF (3 mL) at 0° C. was added DIAD (77 μl, 0.398 mmol), methyl 2-(2-hydroxyphenyl)acetate (CAS #22446-37-3) (76 mg, 0.460 mmol) and then triphenylphosphine (104 mg, 0.398 mmol) and stirred for 16 hours. The reaction was diluted with water and EA, the layers separated and the organic layer was removed, dried (sodium sulfate), concentrated and absorbed onto silica to purify via FCC (0-40% EA:heptanes) to obtain the title compound. MS (ESI+) m/z 435.3, 437.2 (M+H).

### Intermediate 74

### Methyl 2-(2-((3-bromo-5-methoxybenzyl)oxy)phenyl)acetate

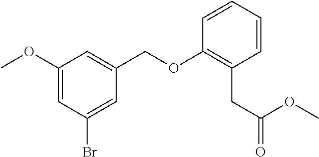

The title compound was synthesized in the same manner as Intermediate 73 using methanol in place of (R)-(tetrahydrofuran-2-yl)methanol (CAS #22415-59-4) in step Intermediate 73-A. MS (ESI+) m/z 364.9, 367.0 (M+H).

### Intermediate 75

### Intermediate 75-A

### Methyl 3-bromo-5-(cyclopropylmethoxy)benzoate

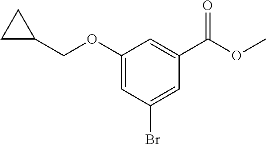 [SMILES: COC(=O)c1cc(Br)cc(OCC2CC2)c1]

The title compound was synthesized as described in Intermediate 55 starting with methyl 3-bromo-5-hydroxybenzoate (Intermediate 57-A) and cyclopropylmethanol. ¹H NMR (400 MHz, DMSO-d₆) δ ppm 7.61 (t, J=1.52 Hz, 1H) 7.42-7.45 (m, 1H) 7.41 (dd, J=2.40, 1.39 Hz, 1H) 3.91 (d, J=7.07 Hz, 2H) 3.86 (s, 3H) 1.15-1.28 (m, 1H) 0.55-0.61 (m, 2H) 0.31-0.37 (m, 2H).

### Intermediate 75-B

### (3-Bromo-5-(cyclopropylmethoxy)phenyl)methanol

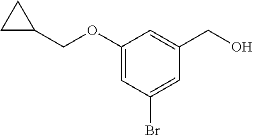 [SMILES: OCc1cc(Br)cc(OCC2CC2)c1]

The title compound was synthesized in the same manner as Intermediate 73-B starting from methyl 3-bromo-5-(cyclopropylmethoxy)benzoate. MS (ESI+) m/z 256.8, 258.7 (M+H).

### Intermediate 75-C

### Methyl 2-(2-((3-bromo-5-(cyclopropylmethoxy)benzyl)oxy)phenyl)acetate

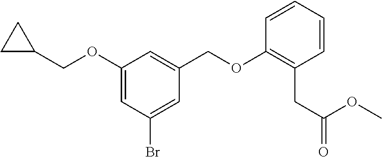 [SMILES: COC(=O)Cc1ccccc1OCc1cc(Br)cc(OCC2CC2)c1]

The title compound was synthesized as described in Intermediate 55 using (3-bromo-5-(cyclopropylmethoxy)phenyl)methanol and methyl 2-(2-hydroxyphenyl)acetate (CAS #22446-37-3). MS (ESI+) m/z 404.8, 406.8 (M+H).

### Intermediate 76

### (R)-Methyl 2-(2-((3-((tetrahydrofuran-2-yl)methoxy)-5-(4,4,5,5-tetramethyl-1,3,2-dioxaborolan-2-yl)benzyl)oxy)phenyl)acetate

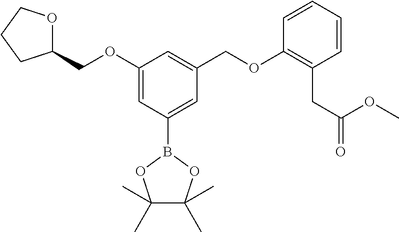 [SMILES: COC(=O)Cc1ccccc1OCc1cc(OC[C@H]2CCCO2)cc(B2OC(C)(C)C(C)(C)O2)c1]

A degassed mixture of (R)-methyl 2-(2-((3-bromo-5-((tetrahydrofuran-2-yl)methoxy)benzyl)oxy)phenyl)acetate (Intermediate 73-C) (0.55 g, 1.263 mmol), 4,4,4′,4′,5,5,5′,5′-octamethyl-2,2′-bi(1,3,2-dioxaborolane) (CAS #73183-34-3) (0.802 g, 3.16 mmol), potassium acetate (0.310 g, 3.16 mmol) and PdCl₂(dppf).CH₂Cl₂ adduct (CAS #95464-05-4) (0.055 g, 0.076 mmol) in DCE (6.32 mL) was heated at 80° C. for 2 hours. The reaction was cooled to room temperature and then loaded onto a silica column to purify via FCC (0-50% EA:heptanes) to obtain the title compound. MS (ESI+) m/z 483.2 (M+H).

### Intermediate 77

### Intermediate 77-A

### 2-Iodo-1-((2-(trimethylsilyl)ethoxy)methyl)-1H-imidazole

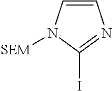 [SMILES: C[Si](C)(C)CCOCn1ccnc1I]

To 2-iodo-1H-imidazole (CAS #3034-62-6) (0.86 g, 4.43 mmol) in THF (22.17 mL) at 0° C. was added NaH (0.213 g, 5.32 mmol) followed by SEMCI (0.944 mL, 5.32 mmol) and the mixture was stirred in an ice bath for 30 minutes and then allowed to warm to room temperature for 2 hours. The mixture was cooled in an ice bath and quenched with aq. solution of ammonium chloride, diluted with EA. The layers were separated and the organic layer dried (sodium sulfate), concentrated and absorbed onto silica to purify via FCC (0-50% EA:heptanes) to obtain the title compound. MS (ESI+) m/z 324.9 (M+H).

### Intermediate 77-B

### 2-(3-Bromophenyl)-1-((2-(trimethylsilyl)ethoxy)methyl)-1H-imidazole

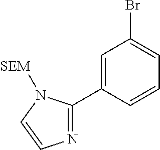 [SMILES: C[Si](C)(C)CCOCn1ccnc1-c1cccc(Br)c1]

In a 2-5 mL microwave vial with stir bar was placed (3-bromophenyl)boronic acid (CAS #189079-40-1) (250 mg, 1.245 mmol) and 2-iodo-1-((2-(trimethylsilyl)ethoxy)methyl)-1H-imidazole (484 mg, 1.494 mmol) in DMF (3.7 mL) and water (0.415 mL). Then, 2M aq. K₃PO₄ (2.5 mL, 4.98 mmol) and PdCl₂(dppf).CH₂Cl₂ adduct (CAS #95464-05-4) (50.8 mg, 0.062 mmol) was added. The vial was sealed and the reaction was heated at 110° C. for 60 min. The reaction mixture was diluted with EA and water and the EA layer was removed, dried and absorbed onto silica to purify via FCC (0-30% EA:heptanes) to obtain the title compound. MS (ESI+) m/z 352.8, 355.2 (M+H).

### Intermediate 78

### Intermediate 78-A

### tert-Butyl 2-(2-((3-bromobenzyl)oxy)phenyl)acetate

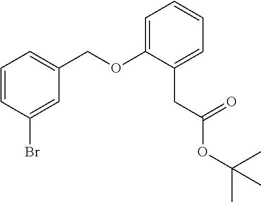 [SMILES: CC(C)(C)OC(=O)Cc1ccccc1OCc1cccc(Br)c1]

To a DMF (21 mL) suspension of tert-butyl 2-(2-hydroxyphenyl)acetate (Intermediate 21) (0.437 g, 2.101 mmol) and K₂CO₃ (0.334 g, 2.416 mmol) was added 3-bromobenzyl bromide (CAS #823-78-9) (0.578 g, 2.311 mmol) and the reaction was warmed to 40° C. overnight for 16 hours. The reaction was concentrated and purified via FCC (0-50% EA:heptanes) to obtain the title compound. MS (ESI+) m/z 377.0, 379.0 (M+H).

### Intermediate 78-B

### tert-Butyl 2-(2-((3-(4,4,5,5-tetramethyl-1,3,2-dioxaborolan-2-yl)benzyl)oxy)phenyl)acetate

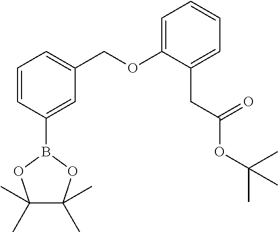 [SMILES: CC(C)(C)OC(=O)Cc1ccccc1OCc1cccc(B2OC(C)(C)C(C)(C)O2)c1]

To tert-butyl 2-(2-((3-bromobenzyl)oxy)phenyl)acetate (0.63 g, 1.670 mmol) in Dioxane (17 ml) was added 4,4,4′,4′,5,5,5′,5′-octamethyl-2,2′-bi(1,3,2-dioxaborolane) (CAS #73183-34-3) (0.594 g, 2.338 mmol), potassium acetate (0.492 g, 5.01 mmol), PdCl₂(dppf).CH₂Cl₂ adduct (CAS #95464-05-4) (0.136 g, 0.167 mmol) and placed under vacuum and purged with nitrogen and then the mixture was heated at 100° C. for 16 hours. The reaction was cooled to room temperature, diluted with EA and water. The EA layer was removed, dried, concentrated and absorbed onto silica to purify via FCC (0-50% EA:heptanes) to obtain the title compound. MS (ESI+) m/z 442.2 (M+H₂O), 369.1 (M−^{t}Bu+H).

### Intermediate 79

### (3-Amino-5-bromo-2-fluorophenyl)methanol

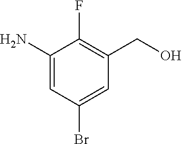 [SMILES: Nc1cc(Br)cc(CO)c1F]

LiBH₄ (220 mg, 10.08 mmol) was added to a mixture of methyl 3-amino-5-bromo-2-fluorobenzoate (CAS #1339049-19-2) (500 mg, 2.016 mmol) in THF (10 mL) at rt, followed by MeOH (0.408 mL, 10.08 mmol). The reaction mixture was stirred at rt for 2 hr. Sat. NH₄Cl was added and the resulting mixture was extracted with EtOAc. The combined organics were washed with brine, dried (Na₂SO₄) and concentrated. The residue was purified by flash chromatography (EtOAc-heptane 0-100%) to provide the title compound. ¹H NMR (400 MHz, CHLOROFORM-d) δ ppm 6.91 (dd, J=5.68, 2.40 Hz, 1H) 6.85 (dd, J=7.64, 2.46 Hz, 1H) 4.69 (s, 2H) 3.79 (br. s., 2H) 1.75 (br. s., 1H).

### Intermediate 80

### Intermediate 80-A

### (3-Amino-5-bromo-4-fluorophenyl)methanol

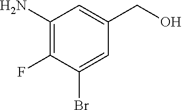 [SMILES: Nc1cc(CO)cc(Br)c1F]

The title compound was synthesized as described in Intermediate 79 starting with methyl 3-amino-5-bromo-4-fluorobenzoate (CAS #1403483-84-0). MS (ESI⁺) m/z 219.9, 221.9 (M+H).

### Intermediate 80-B

### (±)-(3-Bromo-4-fluoro-5-(((tetrahydrofuran-2-yl)methyl)amino)phenyl)methanol

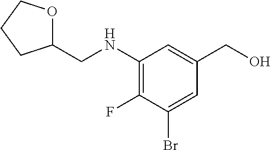 [SMILES: OCc1cc(Br)c(F)c(NCC2CCCO2)c1]

A solution of (3-amino-5-bromo-4-fluorophenyl)methanol (388 mg, 1.763 mmol) and (±)-tetrahydrofuran-2-carbaldehyde (CAS #7681-84-7) (353 mg, 3.53 mmol) in DCE (8 mL) was stirred at rt for 1.5 hr. Na(AcO)₃BH (561 mg, 2.64 mmol) was added and the resulting mixture was stirred at rt overnight. A Sat. aq. solution of NH₄Cl was added and the mixture was extracted with EtOAc. The combined organics were washed with brine, dried (Na₂SO₄) and concentrated. The residue was purified by ISCO (EtOAc-heptane 0-100%) to provide an inseparable mixture of the title compound and starting material, which was further separated by preparative HPLC (method B) to provide the title compound. MS (ESI) m/z 304.0, 306.0 (M+H).

### Intermediate 80-C

### (±)-tert-Butyl 2-(2-((3-bromo-4-fluoro-5-(((tetrahydrofuran-2-yl)methyl)amino)benzyl)oxy)phenyl)acetate

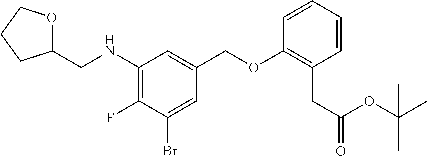 [SMILES: CC(C)(C)OC(=O)Cc1ccccc1OCc1cc(Br)c(F)c(NCC2CCCO2)c1]

DIAD (0.111 mL, 0.572 mmol) was added dropwise to a solution of (3-bromo-4-fluoro-5-(((tetrahydrofuran-2-yl)methyl)amino)phenyl)methanol (145 mg, 0.477 mmol), tert-butyl 2-(2-hydroxyphenyl)acetate (Intermediate 21) and PPh₃ (150 mg, 0.572 mmol) in THF (5 mL) at 0° C. The resulting solution was allowed to warm to room temperature and then stirred for 3 hr. Sat. NH₄Cl was added and the mixture was extracted with EtOAc. The combined organics were washed with brine, dried (Na₂SO₄) and concentrated. The residue was purified by flash chromatography (EtOAc-heptane 0-50%) to provide the title compound. MS (ESI) m/z 494.1, 496.1 (M+H).

### Intermediate 81

### Intermediate 81-A

### tert-Butyl 3-(6-formylpyridin-2-yl)benzylcarbamate

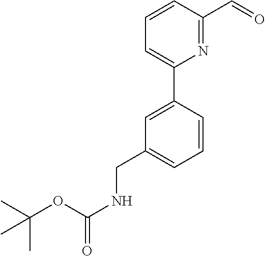 [SMILES: CC(C)(C)OC(=O)NCc1cccc(-c2cccc(C=O)n2)c1]

A vial was charged with (3-(((tert-butoxycarbonyl)amino)methyl)phenyl)boronic acid (CAS #832114-05-3) (562 mg, 2.240 mmol), potassium carbonate (774 mg, 5.60 mmol), 6-bromopicolinaldehyde (CAS #34160-40-2) (500 mg, 2.69 mmol) and PdCl₂(PPh₃)₂ (79 mg, 0.112 mmol). DME (5 mL) and water (0.556 mL) were added, the head space was flushed with nitrogen and the mixture was heated at 90° C. After stirring overnight the mixture was partitioned between ethyl acetate and water. The organic phase was washed with water, brine and dried over sodium sulfate. The residue was purified by flash chromatography (10-80% EtOAc:Heptanes) to afford the title compound. ¹H NMR (400 MHz, DMSO-d₆) δ ppm 10.07 (s, 1H), 8.25 (d, J=7.3 Hz, 1H), 8.15 (t, J=7.8 Hz, 1H), 8.08 (s, 1H), 8.03 (d, J=7.8 Hz, 1H), 7.90 (d, J=6.9 Hz, 1H), 7.53-7.34 (m, 3H), 4.24 (d, J=6.1 Hz, 2H), 1.41 (s, 9H).

### Intermediate 81-B

### tert-Butyl 3-(6-(hydroxymethyl)pyridin-2-yl)benzylcarbamate

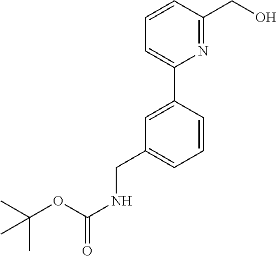 [SMILES: CC(C)(C)OC(=O)NCc1cccc(-c2cccc(CO)n2)c1]

Sodium borohydride (33.9 mg, 0.896 mmol) was added in one portion to a methanol (5 mL) solution of tert-butyl 3-(6-formylpyridin-2-yl)benzylcarbamate (280 mg, 0.896 mmol). After stirring for one hour at room temperature, the reaction was concentrated, partitioned between ethyl acetate and water, washed with brine and dried (sodium sulfate). Flash chromatography (10-90% EtOAc: Heptanes) afforded the title compound. ¹H NMR (400 MHz, DMSO-d₆) δ ppm 7.96 (s, 1H), 7.93-7.85 (m, 2H), 7.77 (d, J=7.8 Hz, 1H), 7.48-7.39 (m, 3H), 7.29 (d, J=7.5 Hz, 1H), 5.43 (t, J=5.9 Hz, 1H), 4.63 (d, J=5.8 Hz, 2H), 4.20 (d, J=5.9 Hz, 2H), 1.41 (s, 9H).

### Intermediate 82

### tert-Butyl ((3′-(hydroxymethyl)-[1,1′-biphenyl]-3-yl)methyl)carbamate

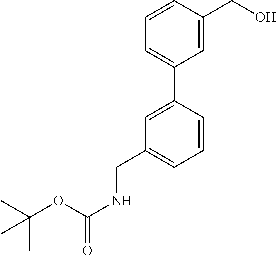 [SMILES: CC(C)(C)OC(=O)NCc1cccc(-c2cccc(CO)c2)c1]

The title compound was synthesized as described in Intermediate 81 starting with 3-bromobenzaldehyde (CAS #3132-99-8). ¹H NMR (400 MHz, DMSO-d₆) δ ppm 7.58 (s, 1H), 7.54-7.47 (m, 3H), 7.45-7.37 (m, 3H), 7.31 (d, J=7.6 Hz, 1H), 7.23 (d, J=7.3 Hz, 1H), 5.23 (t, J=5.7 Hz, 1H), 4.57 (d, J=5.8 Hz, 2H), 4.20 (d, J=6.1 Hz, 2H), 1.40 (s, 9H).

### Intermediate 83

### Intermediate 83-A

### Methyl 6-bromo-1-tosyl-1H-indazole-4-carboxylate

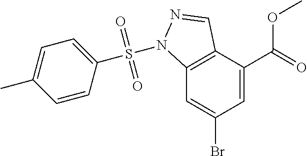

Concentrated HCl (40.3 mL, 1328 mmol) was added to a MeOH (100 mL) suspension of 6-bromoindazole-4-carboxylic acid (CAS #885523-08-0) (4 g, 16.59 mmol) and the resulting suspension was heated for 4 days at 50° C. After cooling to room temperature, the precipitate that was formed was filtered and dried in vacuo to afford the title compound. MS (ESI+) m/z 408.9, 410.9 (M+H).

### Intermediate 83-B

### (6-Bromo-1-tosyl-1H-indazol-4-yl)methanol

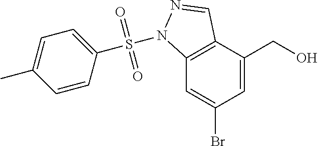 [SMILES: Cc1ccc(S(=O)(=O)n2ncc3c(CO)cc(Br)cc32)cc1]

Lithium borohydride (1.063 mL, 2.126 mmol, 2M in THF) was added at room temperature to a diethyl ether (10 mL) suspension of methyl 6-bromo-1-tosyl-1H-indazole-4-carboxylate (580 mg, 1.417 mmol) and methanol (0.086 mL, 2.126 mmol). After the suspension was stirred overnight, the mixture was partitioned between ethyl acetate and water. The organic phase was washed with brine, dried over sodium sulfate, concentrated and the residue was purified by flash chromatography (0-50% ethyl acetate/heptane) to provide the title compound. MS (ESI+) m/z 380.9, 382.9 (M+H).

### Intermediate 84

The following compounds were prepared with similar methods as described in Intermediate 55 using the appropriate alcohol and phenol starting materials.

| | Structure/Chemical Name | Alcohol | Phenol | MS or NMR data |
|---|---|---|---|---|
| 84-A | 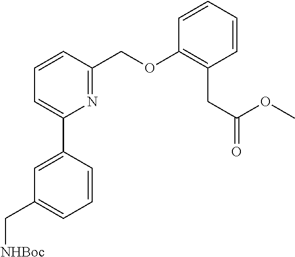 [SMILES: COC(=O)Cc1ccccc1OCc1cccc(-c2cccc(CNC(=O)OC(C)(C)C)c2)n1] | tert-butyl 3-(6- (hydroxymethyl) pyridin-2-yl) benzylcarbamate (Intermediate 81-B) | methyl 2-(2- hydroxyphenyl) acetate (CAS #22446-37-3) | MS (ESI+) m/z 463.4 (M + H). |
| | Methyl 2-(2-((6-(3-(((tert- | | | |
| | butoxycarbonyl)amino)methyl)phenyl)pyridin- | | | |
| | 2-yl)methoxy)phenyl)acetate | | | |
| 84-B | 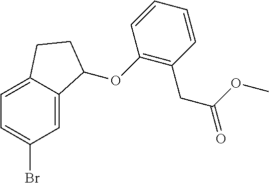 [SMILES: COC(=O)Cc1ccccc1OC1CCc2ccc(Br)cc21] | 6-bromo-2,3- dihydro-1H- inden-1-ol (CAS # 75476-86-7) | methyl 2-(2- hydroxyphenyl) acetate | MS (ESI−) m/z 359.0, 361.0 (M − H). |
| | (±)-Methyl 2-(2-((6-bromo-2,3- | | | |
| | dihydro-1H-inden-1- | | | |
| | yl)oxy)phenyl)acetate | | | |
| 84-C | 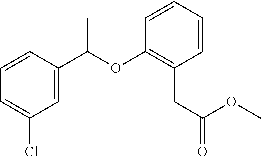 [SMILES: COC(=O)Cc1ccccc1OC(C)c1cccc(Cl)c1] | 1-(3- chlorophenyl) ethanol (CAS #6939- 95-3) | methyl 2-(2- hydroxyphenyl) acetate | MS (ESI+) m/z 305.0 (M + H). |
| | (±)-Methyl 2-(2-(1-(3- | | | |
| | chlorophenyl)ethoxy)phenyl) | | | |
| | acetate | | | |
| 84-D | 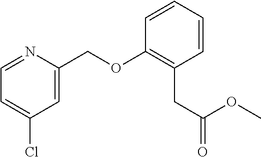 [SMILES: COC(=O)Cc1ccccc1OCc1cc(Cl)ccn1] | (4-chloropyridin- 2-yl)methanol (CAS # 63071-10-3) | methyl 2-(2- hydroxyphenyl) acetate | MS (ESI+) m/z 292.0 (M + H). |
| | Methyl 2-(2-((4-chloropyridin- | | | |
| | 2-yl)methoxy)phenyl)acetate | | | |
| 84-E | 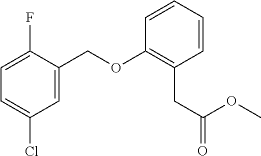 | (5-bromo-2- fluorophenyl) methanol (CAS # 99725-13-0) | methyl 2-(2- hydroxyphenyl) acetate | MS (ESI+) m/z 352.8, 354.8 (M + H). |
| | Methyl 2-(2-((5-bromo-2- | | | |
| | fluorobenzyl)oxy)phenyl)acetate | | | |
| 84-F | 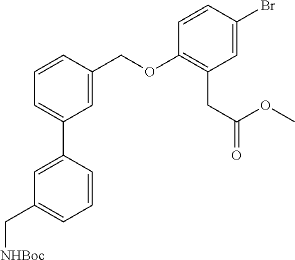 [SMILES: COC(=O)Cc1cc(Br)ccc1OCc1cccc(-c2cccc(CNC(=O)OC(C)(C)C)c2)c1] | tert-butyl ((3′- (hydroxymethyl)- [1,1′-biphenyl]- 3-yl)methyl) carbamate (Intermediate 82) | methyl 2-(5- bromo-2- hydroxyphenyl) acetate (CAS # 220801-66-1) | MS (ESI+) m/z 539.7, 541.7 (M + H). |
| | methyl 2-(5-bromo-2-((3′-(((tert- | | | |
| | butoxycarbonyl)amino) methyl)-[1,1′- | | | |
| | biphenyl]-3-yl)methoxy)phenyl)acetate | | | |
| 84-G | 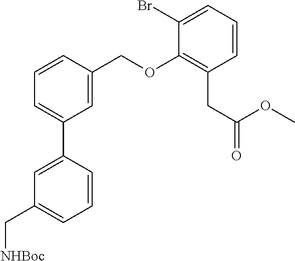 [SMILES: COC(=O)Cc1cccc(Br)c1OCc1cccc(-c2cccc(CNC(=O)OC(C)(C)C)c2)c1] | tert-butyl ((3′- (hydroxymethyl)- [1,1′-biphenyl]-3- yl)methyl) carbamate (Intermediate 82) | methyl 2-(3- bromo-2- hydroxyphenyl) acetate (CAS # 628331-74- 8) | MS (ESI+) m/z 440.0, 442.0 (M − Boc + H). |
| | Methyl 2-(3-bromo-2-((3′-(((tert- | | | |
| | butoxycarbonyl)amino)methyl)-[1,1′- | | | |
| | biphenyl]-3-yl)methoxy)phenyl)acetate | | | |
| 84-H | 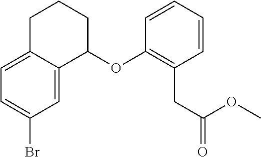 [SMILES: COC(=O)Cc1ccccc1OC1CCCc2ccc(Br)cc21] | 7-bromo- 1,2,3,4- tetrahydro- naphthalen-1-ol (CAS # 75693-15-1) | methyl 2-(2- hydroxyphenyl) acetate | MS (ESI−) m/z 373.0, 375.0 (M − H). |
| | (±)-Methyl 2-(2-((7-bromo-1,2,3,4- | | | |
| | tetrahydronaphthalen-1- | | | |
| | yl)oxy)phenyl)acetate | | | |
| 84-I | 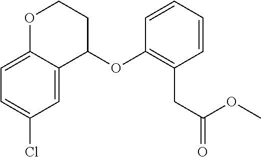 [SMILES: COC(=O)Cc1ccccc1OC1CCOc2ccc(Cl)cc21] | 6- chlorochroman- 4-ol (CAS # 18385-76-7) | methyl 2-(2- hydroxyphenyl) acetate | MS (ESI−) m/z 331.1 (M − H). |
| | (±)-Methyl 2-(2-((6- | | | |
| | chlorochroman-4- | | | |
| | yl)oxy)phenyl)acetate | | | |
| 84-J | 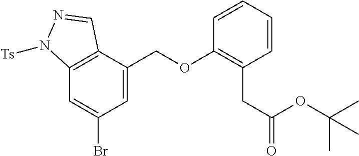 [SMILES: Cc1ccc(S(=O)(=O)n2ncc3c(COc4ccccc4CC(=O)OC(C)(C)C)cc(Br)cc32)cc1] | (6-bromo-1- tosyl-1H- indazol-4- yl)methanol (Intermediate 83-B) | tert-butyl 2-(2- hydroxyphenyl) acetate (Intermediate 21) | MS (ESI+) m/z 516.9, 514.9 (M − tBu + H). |
| | tert-Butyl 2-(2-((6-bromo-1- | | | |
| | tosyl-1H-indazol-4- | | | |
| | yl)methoxy)phenyl)acetate | | | |
| 84-K | 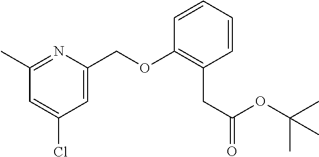 [SMILES: Cc1cc(Cl)cc(COc2ccccc2CC(=O)OC(C)(C)C)n1] | (4-chloro-6- methylpyridin-2- yl)methanol (CAS # 98280-32-1) | tert-butyl 2-(2- hydroxyphenyl) acetate (Intermediate 21) | ¹H NMR (400 MHz, DMSO-d₆) δ ppm 7.39 (d, J = 1.0 Hz, 2H), 7.27-7.20 (m, 2H), 7.01 (d, J = 7.7 Hz, 1H), 6.93 (dt, J = 1.0, 7.4 Hz, 1H), 5.14 (s, 2H), 3.60 (s, 2H), 2.49 (s, 3H), |
| | tert-Butyl 2-(2-((4-chloro-6- | | | 1.35 (s, 9H) |
| | methylpyridin-2- | | | |
| | yl)methoxy)phenyl)acetate | | | |
| 84-L | 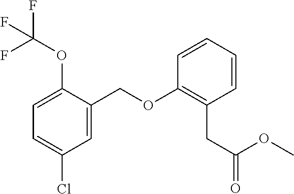 [SMILES: COC(=O)Cc1ccccc1OCc1cc(Cl)ccc1OC(F)(F)F] | (5-chloro-2- (trifluoromethoxy) phenyl)methanol (CAS # 261763-21-7) | methyl 2-(2- hydroxyphenyl) acetate | MS (ESI+) m/z 375.0 (M + H). |
| | Methyl 2-(2-((5-chloro-2- | | | |
| | (trifluoromethoxy)benzyl)oxy) | | | |
| | phenyl)acetate | | | |
| 84-M | 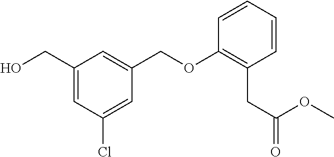 [SMILES: COC(=O)Cc1ccccc1OCc1cc(Cl)cc(CO)c1] | (5-chloro-1,3- phenylene) dimethanol (CAS # 1000342-26- 6) | methyl 2-(2- hydroxyphenyl) acetate | MS (ESI+) m/z 321.1 (M + H). |
| | Methyl 2-(2-((3-chloro-5- | | | |
| | (hydroxymethyl)benzyl)oxy) | | | |
| | phenyl)acetate | | | |
| 84-N | 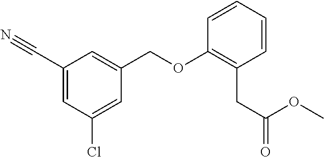 [SMILES: COC(=O)Cc1ccccc1OCc1cc(Cl)cc(C#N)c1] | 3-chloro-5- (hydroxymethyl) benzonitrile (CAS # 1021871-35-1) | methyl 2-(2- hydroxyphenyl) acetate | MS (ESI+) m/z 316.0 (M + H). |
| | Methyl 2-(2-((3-chloro-5- | | | |
| | cyanobenzyl)oxy)phenyl)acetate | | | |
| 84-O | 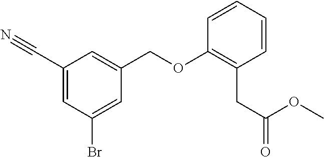 [SMILES: COC(=O)Cc1ccccc1OCc1cc(Br)cc(C#N)c1] | 3-bromo-5- (hydroxymeth yl)benzonitrile (CAS # 1205515-06-5) | methyl 2-(2- hydroxyphenyl) acetate | MS (ESI−) m/z 357.9, 369.9 (M + H). |
| | Methyl 2-(2-((3-bromo-5- | | | |
| | cyanobenzyl)oxy)phenyl)acetate | | | |

### Intermediate 85

### Methyl 2-(2-((5-chloro-2-(trifluoromethyl)benzyl)oxy)phenyl)acetate

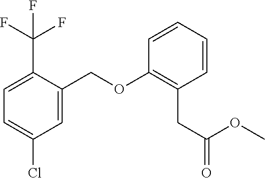 [SMILES: COC(=O)Cc1ccccc1OCc1cc(Cl)ccc1C(F)(F)F]

The title compound was synthesized as described in Intermediate 52 starting with 2-(bromomethyl)-4-chloro-1-(trifluoromethyl)benzene (CAS #261763-21-7) and methyl 2-(2-hydroxyphenyl)acetate (CAS #22446-37-3). MS (ESI) m/z 359.1 (M+H).

### Intermediate 86

### (±)-Methyl 2-(2-((6-(4,4,5,5-tetramethyl-1,3,2-dioxaborolan-2-yl)-2,3-dihydro-1H-inden-1-yl)oxy)phenyl)acetate

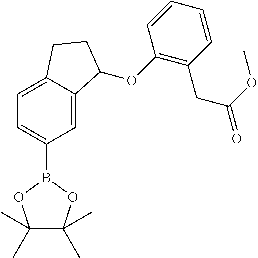 [SMILES: COC(=O)Cc1ccccc1OC1CCc2ccc(B3OC(C)(C)C(C)(C)O3)cc21]

The title compound was synthesized as described in Intermediate 24 starting with (±)-methyl 2-(2-((6-bromo-2,3-dihydro-1H-inden-1-yl)oxy)phenyl)acetate (Intermediate 84-B). ¹H NMR (400 MHz, DMSO-d₆) δ ppm 7.64-7.60 (m, 2H), 7.36-7.27 (m, 2H), 7.22-7.18 (m, 2H), 6.91 (dt, J=1.0, 7.4 Hz, 1H), 5.83 (dd, J=4.9, 6.4 Hz, 1H), 3.51 (s, 3H), 3.49 (d, J=5.4 Hz, 2H), 3.09-3.00 (m, 1H), 2.91 (s, 1H), 2.59 (d, J=6.8 Hz, 1H), 1.94 (d, J=6.1 Hz, 1H), 1.27 (d, J=2.1 Hz, 12H).

### Intermediate 87

### (±)-Methyl 2-(2-((7-(4,4,5,5-tetramethyl-1,3,2-dioxaborolan-2-yl)-1,2,3,4-tetrahydronaphthalen-1-yl)oxy)phenyl)acetate

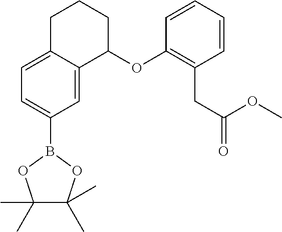

The title compound was synthesized as described in Intermediate 24 starting with methyl 2-(2-((7-bromo-1,2,3,4-tetrahydronaphthalen-1-yl)oxy)phenyl)acetate (Intermediate 84-H). MS (ESI+) m/z 440.2 (M+H₂O).

### Intermediate 88

### (S)-tert-Butyl (2-methoxy-1-(3-(4,4,5,5-tetramethyl-1,3,2-dioxaborolan-2-yl)phenyl)ethyl)carbamate

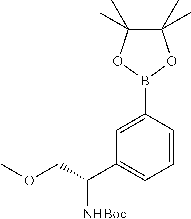

The title compound was synthesized as described in Intermediate 24 starting with (S)-tert-butyl (1-(3-bromophenyl)-2-methoxyethyl)carbamate (Intermediate 36). MS (ESI+) m/z 378.1 (M+H).

### Intermediate 89

### tert-Butyl 2-(2-((3-bromo-5-(cyclopropylmethoxy)benzyl)oxy)phenyl)acetate

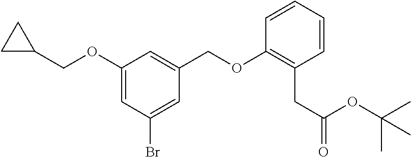 [SMILES: CC(C)(C)OC(=O)Cc1ccccc1OCc1cc(Br)cc(OCC2CC2)c1]

The title compound was synthesized as described in Intermediate 75-C using tert-butyl 2-(2-hydroxyphenyl)acetate (Intermediate 21) in place of methyl 2-(2-hydroxyphenyl)acetate (CAS #22446-37-3). MS (ESI+) m/z 391.0, 393.0 (M−tBu+H).

### Intermediate 90

### methyl 2-(2-((3-bromobenzyl)oxy)-3-fluorophenyl)acetate

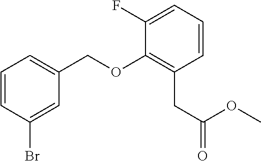 [SMILES: COC(=O)Cc1cccc(F)c1OCc1cccc(Br)c1]

The title compound was synthesized as described in Intermediate 52 starting with 3-bromobenzyl bromide (CAS #823-78-9) and methyl 2-(3-fluoro-2-hydroxyphenyl)acetate (Intermediate 22). MS (ESI+) m/z 352.9, 354.9 (M+H).

### Intermediate 91

### tert-Butyl 2-(2-((3-chloro-5-(4,4,5,5-tetramethyl-1,3,2-dioxaborolan-2-yl)benzyl)oxy)phenyl)acetate

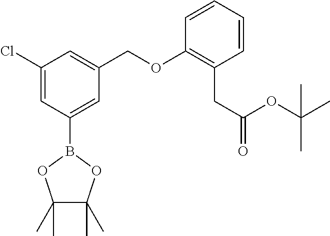 [SMILES: CC(C)(C)OC(=O)Cc1ccccc1OCc1cc(Cl)cc(B2OC(C)(C)C(C)(C)O2)c1]

The title compound was synthesized as described in Intermediate 34-B starting with tert-butyl 2-(2-((3-bromo-5-chlorobenzyl)oxy)phenyl)acetate (Intermediate 53). ¹H NMR (400 MHz, DICHLOROMETHANE-d₂) δ ppm 7.57-7.61 (m, 2H) 7.49 (s, 1H) 7.08-7.18 (m, 2H) 6.81-6.88 (m, 2H) 4.97 (s, 2H) 3.49 (s, 2H) 1.30 (s, 9H) 1.25 (s, 12H).

### Intermediate 92

### tert-Butyl 2-(2-((3-(methoxymethyl)-5-(4,4,5,5-tetramethyl-1,3,2-dioxaborolan-2-yl)benzyl)oxy)phenyl)acetate

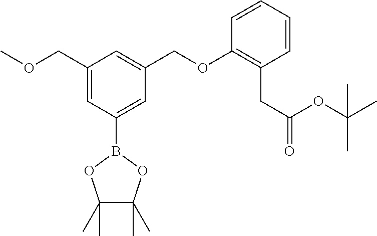 [SMILES: COCc1cc(COc2ccccc2CC(=O)OC(C)(C)C)cc(B2OC(C)(C)C(C)(C)O2)c1]

The title compound was synthesized as described in Intermediate 34-B starting with tert-butyl 2-(2-((3-bromo-5-(methoxymethyl)benzyl)oxy)phenyl)acetate (Intermediate 61). MS (ESI+) m/z 413.2 (M−tBu+H).

### Intermediate 93

### tert-Butyl 2-(2-((3-(cyclopropylmethoxy)-5-(4,4,5,5-tetramethyl-1,3,2-dioxaborolan-2-yl)benzyl)oxy)phenyl)acetate

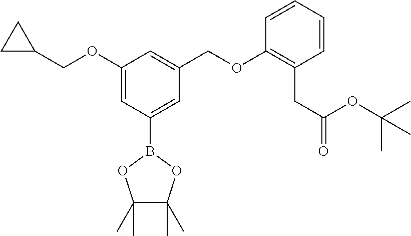 [SMILES: CC(C)(C)OC(=O)Cc1ccccc1OCc1cc(OCC2CC2)cc(B2OC(C)(C)C(C)(C)O2)c1]

The title compound was synthesized as described in Intermediate 34-B starting with tert-butyl 2-(2-((3-bromo-5-(cyclopropylmethoxy)benzyl)oxy)phenyl)acetate (Intermediate 89). MS (ESI+) m/z 439.3 (M−tBu+H).

### Intermediate 94

### Intermediate 94-A

### Methyl 2-(2-((3-chloro-5-formylbenzyl)oxy)phenyl)acetate

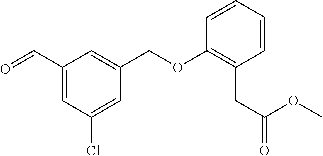 [SMILES: COC(=O)Cc1ccccc1OCc1cc(Cl)cc(C=O)c1]

A solution of DMSO (1.444 mL, 20.34 mmol) in DCM (4 ml) was added dropwise to a solution of oxalyl dichloride (0.872 mL, 10.17 mmol) in DCM (6 ml) cooled down at −70° C. The reaction mixture was then stirred at −70° C. for 30 min. Then a solution of methyl 2-(2-((3-chloro-5-(hydroxymethyl)benzyl)oxy)phenyl)acetate (Intermediate 84-M) in DCM (10 ml) was added dropwise at −70° C. to the mixture. The reaction mixture was kept at this temperature while stirring for 5 h. TEA (4.96 mL, 35.6 mmol) was then added and the reaction mixture was allowed to warm up to room temperature and stirred for 30 min. The reaction was quenched with aq. sat. NaHCO₃ and extracted with DCM. The organic phase was dried (Na₂SO₄), filtered and evaporated. The residue was purified by flash chromatography on silica gel (eluent cyclohexane/ethyl acetate 100:0 to 60:40) to yield the title compound. MS (ESI+) m/z: 319.1 (M+H).

### Intermediate 94-B

### (±)-(E)-Methyl 2-(2-((3-(((tert-butylsulfinyl)imino)methyl)-5-chlorobenzyl)oxy)phenyl)acetate

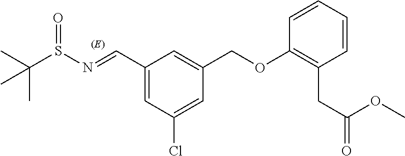 [SMILES: COC(=O)Cc1ccccc1OCc1cc(Cl)cc(/C=N/S(=O)C(C)(C)C)c1]

To a solution of methyl 2-(2-((3-chloro-5-formylbenzyl)oxy)phenyl)acetate (1.27 g, 3.98 mmol) and (±)-2-methylpropane-2-sulfinamide (0.531 g, 4.38 mmol) in toluene (30 mL) was added Ti(OiPr)₄ (1.699 g, 5.98 mmol). The resulting slurry mixture was stirred at 50° C. for 48 hours. The reaction was quenched by addition of water and extracted with ethyl acetate. The organic phase was washed with NaCl, then dried (Na₂SO₄), filtered and evaporated. The residue was purified by flash chromatography on silica gel (eluent cyclohexane/ethyl acetate 100:0 to 70:30) to yield the title compound. MS (ESI+) m/z: 421.9 (M+H), Rf (Cyclohex:EtOAc 8:2)=0.19.

### Intermediate 94-C

### (±)-Methyl 2-(2-((3-chloro-5-(1-(1,1-dimethylethylsulfinamido)-2,2,2-trifluoroethyl)benzyl)oxy)phenyl)acetate

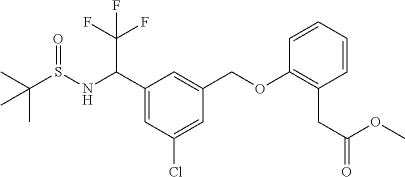 [SMILES: COC(=O)Cc1ccccc1OCc1cc(Cl)cc(C(NS(=O)C(C)(C)C)C(F)(F)F)c1]

A solution of (±)-(E)-methyl 2-(2-((3-(((tert-butylsulfinyl)imino)methyl)-5-chlorobenzyl)oxy)phenyl)-acetate (1.3 g, 3.08 mmol) and TBAT (3.55 g, 6.57 mmol) in dry THF (35 ml) was cooled down at −65° C. under argon atmosphere. To the mixture was added dropwise TMSCF₃ (1.070 ml, 7.17 mmol) solution in 5 ml of THF. The mixture which turned to a white suspension was kept at this temperature and stirred for 2 hours. After 2 h, the mixture was allowed to warm up to 0° C. and then quenched with NH₄Cl. The mixture was stirred for 30 min and then extracted with EtAOc. The combined organic layers were dried (Na₂SO₄), filtered and and evaporated. The residue was purified by flash chromatography on silica gel (eluent cyclohexane/ethyl acetate 100:0 to 40:60) to yield the title compound. MS (ESI+) m/z: 492.1 (M+H).

### Intermediate 95

### (±)-Methyl 2-(2-((3′-(((tert-butoxycarbonyl)amino)methyl)-5-(1-(1,1-dimethylethylsulfinamido)-2,2,2-trifluoroethyl)-[1,1′-biphenyl]-3-yl)methoxy)phenyl)acetate

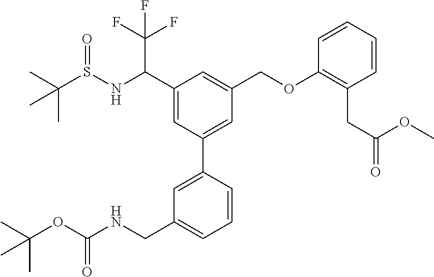 [SMILES: COC(=O)Cc1ccccc1OCc1cc(-c2cccc(CNC(=O)OC(C)(C)C)c2)cc(C(NS(=O)C(C)(C)C)C(F)(F)F)c1]

To a solution of methyl 2-(2-((3-chloro-5-(1-(1,1-dimethylethylsulfinamido)-2,2,2-trifluoroethyl)benzyl)oxy)phenyl)acetate (30 mg, 0.061 mmol) and (3-(((tert-butoxycarbonyl)amino)methyl)phenyl)boronic acid (CAS #832114-05-3) (18.37 mg, 0.073 mmol) in dioxane (2.0 ml) was added Pd₂(dba)₃ (0.558 mg, 0.610 μmol), X-Phos (CAS #564483-18-7) (1.163 mg, 2.439 μmol) and CsF (27.8 mg, 0.183 mmol). The reaction mixture was heated in the microwave oven at 120° C. for 30 min. The black mixture is diluted in DCM and washed with aq. sat. NaHCO₃ and then water. The organic layer was separated, filtered over a phase separating cartridge and evaporated. The residue is purified by flash chromatography on silica gel (eluent cyclohexane/ethyl acetate 100:0 to 60:40) to yield the title compound. MS (ESI+) m/z: 663.3 (M+H).

### Intermediate 96

### (±)-Methyl 2-(2-((3-(1-amino-2,2,2-trifluoroethyl)-5-chlorobenzyl)oxy)phenyl)acetate

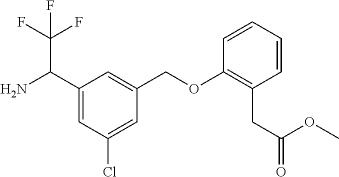 [SMILES: COC(=O)Cc1ccccc1OCc1cc(Cl)cc(C(N)C(F)(F)F)c1]

To a solution of methyl 2-(2-((3-chloro-5-(1-(1,1-dimethylethylsulfinamido)-2,2,2-trifluoroethyl)benzyl)oxy)phenyl)acetate (Intermediate 94-C) (1.20 g, 2.44 mmol) in methanol (25 mL) was added HCl 4M in Dioxane (1.220 mL, 4.88 mmol) and the reaction mixture was stirred at 23° C. for 1 hour. The mixture was concentrated to dryness. The oily residue was then dissolved in methylene chloride and washed with a sat. solution of sodium bicarbonate. The aqueous phase was extracted with methylene chloride. The combined organic phases were dried over a phase separating cartridge and concentrated to afford crude title compound which was used in subsequent steps without further purification. MS (ESI+) m/z: 388.1 (M+H).

### Intermediate 97

### Intermediate 97-A

### (±)-Methyl 2-(2-((3-chloro-5-(2,2,2-trifluoro-1-(methylamino)ethyl)benzyl oxy)phenyl)acetate

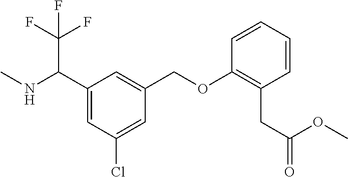

To a solution of methyl 2-(2-((3-(1-amino-2,2,2-trifluoroethyl)-5-chlorobenzyl)oxy)phenyl)acetate (Intermediate 96) (250 mg, 0.026 mmol) in ethyl acetate (2.0 ml) was added sodium bicarbonate (271 mg, 3.22 mmol) solution in water (1.0 ml) followed by methyl trifluoromethanesulfonate (116 mg, 0.709 mmol) solution in EtOAc (1.0 ml). The reaction mixture was stirred at room temperature for 1 hour. The organic phase was then extracted, dried (Na₂SO₄), filtered and evaporated. The residue was purified by flash chromatography on silica gel (eluent cyclohexane/ethyl acetate 100:0 to 50:50) to yield the title compound. MS (ESI+) m/z: 402.1 (M+H), HPLC (method C): 1.864 min, Rf (Cyclohexane/EtOAc 75:25): 0.37.

### Intermediate 97-B

### (±)-Methyl 2-(2-((3-chloro-5-(1-(dimethylamino)-2,2,2-trifluoroethyl)benzyl)oxy)phenyl)acetate

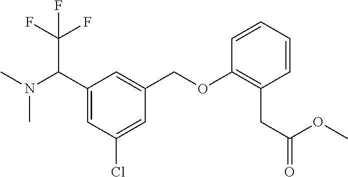

The title compound was obtained as side product in the reaction described in Intermediate 97-A. MS (ESI+) m/z: 416.1 (M+H), HPLC (method C): 2.176 min, Rf (Cyclohexane/EtOAc 75:25): 0.53.

### Intermediate 98

### (±)-Methyl 2-(2-((3-chloro-5-(2,2,2-trifluoro-1-(phenylamino)ethyl)benzyl)oxy)phenyl)acetate

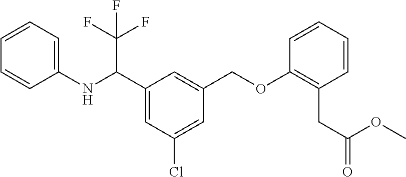 [SMILES: COC(=O)Cc1ccccc1OCc1cc(Cl)cc(C(Nc2ccccc2)C(F)(F)F)c1]

A mixture of methyl 2-(2-((3-(1-amino-2,2,2-trifluoroethyl)-5-chlorobenzyl)oxy)phenyl)acetate (Intermediate 96) (200 mg, 0.516 mmol), bromobenzene (121 mg, 0.774 mmol) and Cs₂CO₃ (336 mg, 1.032 mmol) in toluene (10 ml) was purged under argon atmosphere for 5 min, then was added X-Phos (CAS #564483-18-7) (49.2 mg, 0.103 mmol) and Pd(OAc)₂ (11.58 mg, 0.052 mmol). The reaction mixture was stirred at 100° C. for 16 hours. After cooling to room temperature, the reaction was quenched with aq. sat. NaHCO₃ and extracted with ethyl acetate. The organic phase was dried (Na₂SO₄), filtered and evaporated and subsequently purified by flash chromatography on silica gel (eluent cyclohexane/ethyl acetate 100:0 to 65:35) to yield the title compound as a colorless oil. MS (ESI+) m/z: 464.1 (M+H).

### Intermediate 99

### (±)-Methyl 2-(2-((3-(1-benzamido-2,2,2-trifluoroethyl)-5-chlorobenzyl)oxy)phenyl)acetate

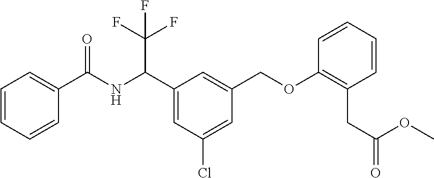 [SMILES: COC(=O)Cc1ccccc1OCc1cc(Cl)cc(C(NC(=O)c2ccccc2)C(F)(F)F)c1]

To a solution of methyl 2-(2-((3-(1-amino-2,2,2-trifluoroethyl)-5-chlorobenzyl)oxy)phenyl)acetate (Intermediate 96) (18 mg, 0.046 mmol) in DCM (1.0 ml) was added benzoyl chloride (6.47 μl, 0.056 mmol) followed by TEA (7.76 μl, 0.056 mmol). The mixture was stirred for 1 hour at room temperature, then quenched by addition of water. The organic phase was extracted, dried (sodium sulfate) and evaporated. The residue was purified by flash chromatography on silica gel (eluent cyclohexane/ethyl acetate 100:0 to 60:40) to yield the title compound as a colorless oil. MS (ESI+) m/z: 492.1 (M+H).

### Intermediate 100

### Intermediate 100-A

### Methyl 2-(2-((3-(aminomethyl)-5-chlorobenzyl)oxy)phenyl)acetate

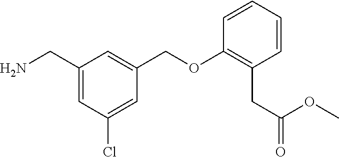 [SMILES: COC(=O)Cc1ccccc1OCc1cc(Cl)cc(CN)c1]

To a solution of methyl 2-(2-((3-chloro-5-cyanobenzyl)oxy)phenyl)acetate (Intermediate 84-N) (1.27 g, 4.02 mmol) and CoCl₂.6H₂O (1.435 g, 6.03 mmol) in THF (30 ml)/Water (10.7 ml) cooled down at 0° C. was added portionwise NaBH₄ (457 mg, 12.07 mmol) under argon atmosphere. The reaction mixture was stirred at 0° C. for 2 hours. The reaction mixture was then quenched with aq. sat. Na₂CO₃ and extracted three times with DCM. The combined organic phases were dried (cartridge), evaporated and subsequently purified by flash chromatography on silica gel (eluents cyclohexane/ethyl acetate 100:0 to 0:100 and then DCM/DCM:MeOH 9: 100:0 to 0:100) to yield the title compound. MS (ESI+) m/z: 320.1 (M+H).

### Intermediate 100-B

### Methyl 2-(2-((3-chloro-5-(((2,2,2-trifluoroethyl)amino)methyl)benzyl)oxy)phenyl)acetate

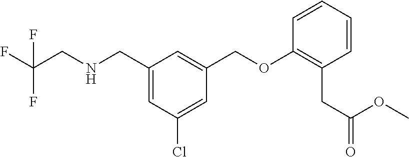 [SMILES: COC(=O)Cc1ccccc1OCc1cc(Cl)cc(CNCC(F)(F)F)c1]

A mixture of methyl 2-(2-((3-(aminomethyl)-5-chlorobenzyl)oxy)phenyl)acetate (220 mg, 0.688 mmol) and 1-ethoxy-2,2,2-trifluoroethanol (0.156 ml, 1.376 mmol) was heated at 105° C. After 3 hours, the mixture was cooled down to room temperature, dissolved in MeOH (6 ml) and cooled to 0° C. with an ice-bath. NaBH₄ (52.1 mg, 1.376 mmol) was then slowly added under argon atmosphere and the reaction was heated at 50° C. overnight. The reaction was quenched by addition of NH₄Cl solution and extracted with DCM. The organic phase was washed with aq. sat. NaHCO₃, then dried (cartridge) and evaporated. The residue was purified by flash chromatography on silica gel (eluent cyclohexane/ethyl acetate 100:0 to 57:43) to yield the title compound. MS (ESI+) m/z: 402.1 (M+H).

### Intermediate 101

### (±)-Methyl 2-(2-((3-chloro-5-(((1,1,1-trifluoropropan-2-yl)amino)methyl)benzyl)oxy)phenyl)acetate

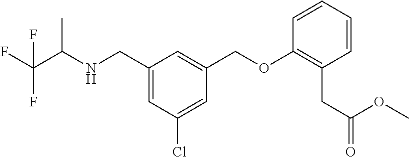 [SMILES: COC(=O)Cc1ccccc1OCc1cc(Cl)cc(CNC(C)C(F)(F)F)c1]

To a solution of methyl 2-(2-((3-chloro-5-formylbenzyl)oxy)phenyl)acetate (Intermediate 94-A, 120 mg, 0.376 mmol) in toluene (6 ml) was added 1,1,1-trifluoropropan-2-amine (85 mg, 0.753 mmol) followed by Ti(OiPr)₄ (214 mg, 0.753 mmol). The reaction mixture was stirred at 45° C. overnight before being cooled down to room temperature and diluted in EtOAc. A suspension was formed, which filtered and washed several times with EtAOc. The collected filtrate was evaporated. The resulting oily residue was dissolved in MeOH (3 ml) and NaBH₄ (28.5 mg, 0.753 mmol) was added under argon atmosphere. The reaction mixture was stirred at 50° C. for two days before being cooled down to room temperature, diluted in DCM and washed with aq. sat. NaHCO₃. The organic phase was then dried (cartridge), evaporated and purified by Preparative HPLC (Waters Sunfire C18 OBD, 5 μm, 30*100 mm, Eluent A: H₂O+0.1% TFA, B: ACN+0.1% TFA, Gradient: 20% to 100% B in 20 min hold 3 min, Flow 40 mL/min) to yield the title compound. MS (ESI+) m/z: 416.1 (M+H), HPLC (method C): 2.104 min.

### Intermediate 102

### Intermediate 102-A

### Methyl 2-(2-((3-bromo-5-(((2,2,2-trifluoroethyl)amino)methyl)benzyl)oxy)phenyl)acetate

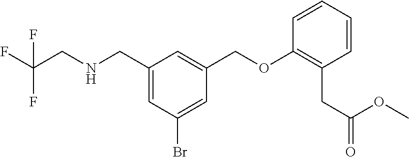 [SMILES: COC(=O)Cc1ccccc1OCc1cc(Br)cc(CNCC(F)(F)F)c1]

Title compound was prepared in analogy to intermediate 100 using intermediate 84-0. MS (ESI+) m/z: 446.1, 448.1 (M+H).

### Intermediate 102-B

### Methyl 2-(2-((3-bromo-5-((methyl(2,2,2-trifluoroethyl)amino)methyl)benzyl)oxy)phenyl)acetate

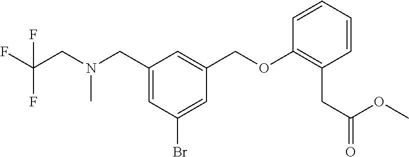 [SMILES: COC(=O)Cc1ccccc1OCc1cc(Br)cc(CN(C)CC(F)(F)F)c1]

To a solution of methyl 2-(2-((3-bromo-5-(((2,2,2-trifluoroethyl)amino)methyl)benzyl)oxy)phenyl)acetate (100 mg, 0.224 mmol) in ethyl acetate (3.0 mL) was added a solution of sodium bicarbonate (94 mg, 1.120 mmol) in water (1 ml) followed by methyl trifluoromethanesulfonate (40.5 mg, 0.246 mmol) solution in EtOAc (1.0 ml). The reaction mixture was stirred at 25° C. for 1 h followed by addition of EtOAc. The organic layer was washed with a saturated solution of bicarbonate, dried over sodium sulfate, filtered and concentrated to dryness to afford a crude oily residue. The residue was purified by flash chromatography using FCC (Cyclohexane/EtOAc, Gradient: 0% to 35% in 15 min). The collected fractions were combined, evaporated and dried over high vacuum to afford title compound. MS (ESI+) m/z: 460.1, 462.1 (M+H).

### Intermediate 103

### Methyl 2-(2-((3-chloro-5-(1-methyl-1H-pyrazol-4-yl)benzyl)oxy)phenyl)acetate

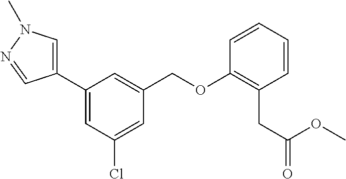 [SMILES: COC(=O)Cc1ccccc1OCc1cc(Cl)cc(-c2cnn(C)c2)c1]

To a solution of methyl 2-(2-((3-bromo-5-chlorobenzyl)oxy)phenyl)acetate (Intermediate 35) (500 mg, 1.353 mmol) and 1-methyl-4-(4,4,5,5-tetramethyl-1,3,2-dioxaborolan-2-yl)-1H-pyrazole (422 mg, 2.029 mmol) in dioxane (12 mL) and water (2 mL) was added PdCl₂(dppf).CH₂Cl₂ adduct (110 mg, 0.135 mmol) and 2M aq. Na₂CO₃ (2.029 mL, 4.06 mmol). The reaction mixture was degassed and heated for 30 min in the microwave to 105° C. The reaction solution was taken up in ethyl acetate and washed with aq. sat. NaHCO₃ solution. The aq. layer was extracted twice with ethyl acetate. The combined organic layer were dried with a phase separator and evaporated to dryness. The crude product was purified by flash-chromatography (silica gel: 24 g, gradient of cyclohexane/EtOAc (35 ml/min.) from 100/0 to 65/35 (35 min.)) to afford title compound. MS (ESI+) m/z: 371.3 (M+H), HPLC (method C): 2.71 min.

### Intermediate 104

The following compounds were prepared with similar methods as described in Intermediate 95 using the appropriate halide and boronic acid or boronic ester as starting materials.

| | | | Boronic | |
|---|---|---|---|---|
| | Structure/Chemical Name | Halide | ester/acid | MS (ESI+) m/z |
| 104-A | 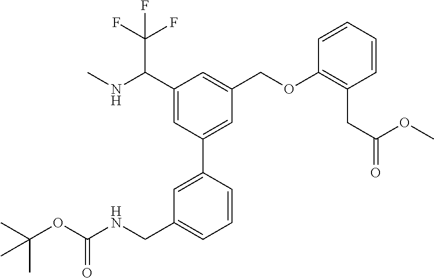 [SMILES: CNC(c1cc(COc2ccccc2CC(=O)OC)cc(-c2cccc(CNC(=O)OC(C)(C)C)c2)c1)C(F)(F)F] | Intermediate 97-A | (3-(((tert- butoxycarbonyl) amino)methyl) phenyl)boronic acid (CAS # 832114-05-3) | 573.2 (M + H). |
| | (±)-Methyl 2-(2-((3′-(((tert- | | | |
| | butoxycarbonyl)amino) | | | |
| | methyl)-5-(2,2,2-trifluoro-1- | | | |
| | (methylamino)ethyl)-[1,1′- | | | |
| | biphenyl]-3- | | | |
| | yl)methoxy)phenyl)acetate | | | |
| 104-B | 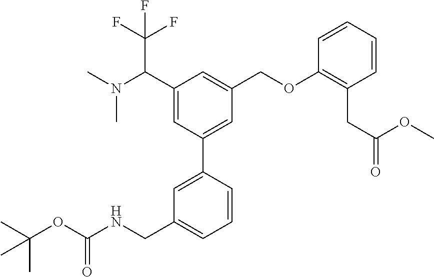 [SMILES: COC(=O)Cc1ccccc1OCc1cc(-c2cccc(CNC(=O)OC(C)(C)C)c2)cc(C(N(C)C)C(F)(F)F)c1] | Intermediate 97-B | (3-(((tert- butoxycarbonyl) amino)methyl) phenyl)boronic acid | 587.2 (M + H). |
| | (±)-Methyl 2-(2-((3′-(((tert- | | | |
| | butoxycarbonyl)amino) | | | |
| | methyl)-5-(1-(dimethylamino)- | | | |
| | 2,2,2-trifluoroethyl)-[1,1′- | | | |
| | biphenyl]-3- | | | |
| | yl)methoxy)phenyl)acetate | | | |
| 104-C | 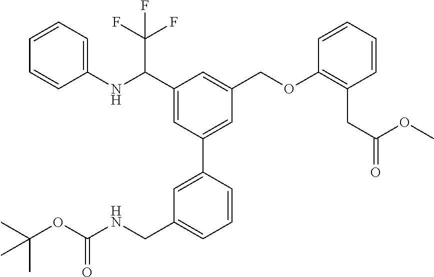 [SMILES: COC(=O)Cc1ccccc1OCc1cc(-c2cccc(CNC(=O)OC(C)(C)C)c2)cc(C(Nc2ccccc2)C(F)(F)F)c1] | Intermediate 98 | (3-(((tert- butoxycarbonyl) amino)methyl) phenyl)boronic acid | 635.2 (M + H) |
| | (±)-Methyl 2-(2-((3′-(((tert- | | | |
| | butoxycarbonyl)amino) | | | |
| | methyl)-5-(2,2,2-trifluoro-1- | | | |
| | (phenylamino)ethyl)-[1,1′- | | | |
| | biphenyl]-3- | | | |
| | yl)methoxy)phenyl)acetate | | | |
| 104-D | 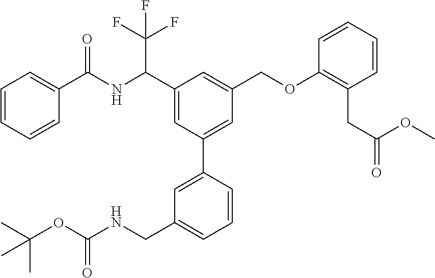 [SMILES: COC(=O)Cc1ccccc1OCc1cc(-c2cccc(CNC(=O)OC(C)(C)C)c2)cc(C(NC(=O)c2ccccc2)C(F)(F)F)c1] | Intermediate 99 | (3-(((tert- butoxycarbonyl) amino)methyl) phenyl)boronic acid | 680.3 (M + NH₃) |
| | (±)-Methyl 2-(2-((5-(1- | | | |
| | benzamido-2,2,2- | | | |
| | trifluoroethyl)-3′-(((tert- | | | |
| | butoxycarbonyl)amino) | | | |
| | methyl)-[1,1′-biphenyl]-3- | | | |
| | yl)methoxy)phenyl)acetate | | | |
| 104-E | 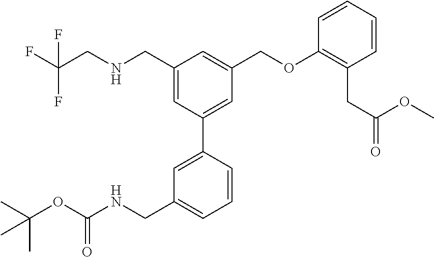 [SMILES: COC(=O)Cc1ccccc1OCc1cc(CNCC(F)(F)F)cc(-c2cccc(CNC(=O)OC(C)(C)C)c2)c1] | Intermediate 100-B | (3-(((tert- butoxycarbonyl) amino)methyl) phenyl)boronic acid | 573.2 (M + H). |
| | Methyl 2-(2-((3′-(((tert- | | | |
| | butoxycarbonyl)amino) | | | |
| | methyl)-5-(((2,2,2- | | | |
| | trifluoroethyl)amino)methyl)- | | | |
| | [1,1′-biphenyl]-3- | | | |
| | yl)methoxy)phenyl)acetate | | | |
| 104-F | 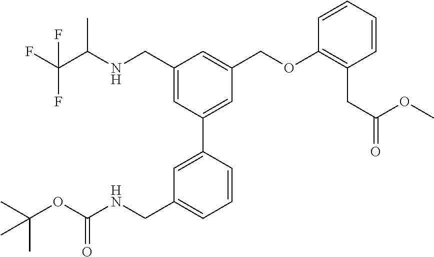 [SMILES: COC(=O)Cc1ccccc1OCc1cc(CNC(C)C(F)(F)F)cc(-c2cccc(CNC(=O)OC(C)(C)C)c2)c1] | Intermediate 101 | (3-(((tert- butoxycarbonyl) amino)methyl) phenyl)boronic acid | 587.3 (M + H). |
| | (±)-Methyl 2-(2-((3′-(((tert- | | | |
| | butoxycarbonyl)amino) | | | |
| | methyl)-5-(((1,1,1- | | | |
| | trifluoropropan-2- | | | |
| | yl)amino)methyl)-[1,1′- | | | |
| | biphenyl]-3- | | | |
| | yl)methoxy)phenyl)acetate | | | |
| 104-G | 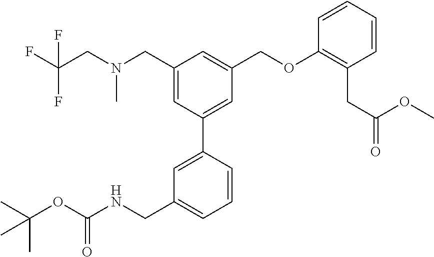 [SMILES: COC(=O)Cc1ccccc1OCc1cc(CN(C)CC(F)(F)F)cc(-c2cccc(CNC(=O)OC(C)(C)C)c2)c1] | Intermediate 102-B | (3-(((tert- butoxycarbonyl) amino)methyl) phenyl)boronic acid | 587.3 (M + H). |
| | Methyl 2-(2-((3′-(((tert- | | | |
| | butoxycarbonyl)amino) | | | |
| | methyl)-5-((methyl(2,2,2- | | | |
| | trifluoroethyl)amino)methyl)- | | | |
| | [1,1′-biphenyl]-3- | | | |
| | yl)methoxy)phenyl)acetate | | | |
| 104-H | 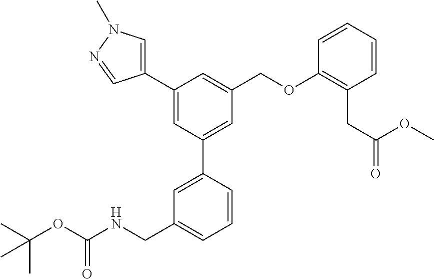 [SMILES: COC(=O)Cc1ccccc1OCc1cc(-c2cccc(CNC(=O)OC(C)(C)C)c2)cc(-c2cnn(C)c2)c1] | Intermediate 103 | (3-(((tert- butoxycarbonyl) amino)methyl) phenyl)boronic acid | 542.2 (M + H). |
| | Methyl 2-(2-((3′-(((tert- | | | |
| | butoxycarbonyl)amino) | | | |
| | methyl)-5-(1-methyl-1H-pyrazol- | | | |
| | 4-yl)-[1,1′-biphenyl]-3- | | | |
| | yl)methoxy)phenyl)acetate | | | |

### Intermediate 105

### (S)-tert-Butyl (1-(2-fluoro-3-(4,4,5,5-tetramethyl-1,3,2-dioxaborolan-2-yl)phenyl)ethyl)carbamate

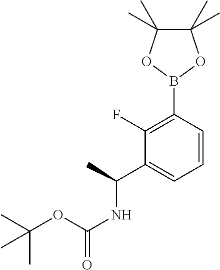 [SMILES: C[C@H](NC(=O)OC(C)(C)C)c1cccc(B2OC(C)(C)C(C)(C)O2)c1F]

The title compound was synthesized as described in Intermediate 27 starting with (S)-1-(3-chloro-2-fluorophenyl)ethanamine hydrochloride (CAS #1313593-59-7). MS (ESI+) m/z 310.2 (M−tBu+H).

### Intermediate 106

### (S)-tert-Butyl (1-(3-(4,4,5,5-tetramethyl-1,3,2-dioxaborolan-2-yl)phenyl)ethyl)carbamate

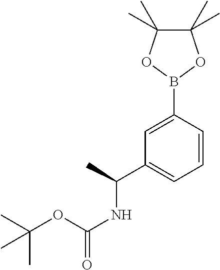 [SMILES: C[C@H](NC(=O)OC(C)(C)C)c1cccc(B2OC(C)(C)C(C)(C)O2)c1]

The title compound was synthesized as described in Intermediate 25 and 26 starting with (S)-1-(3-bromophenyl)ethanamine (CAS #139305-96-7). MS (ESI+) m/z 292.3 (M−tBu+H).

### Intermediate 107

### tert-Butyl 2-(2-((3-bromo-5-((2,2,2-trifluoroethoxy)methyl)benzyl)oxy)phenyl)acetate

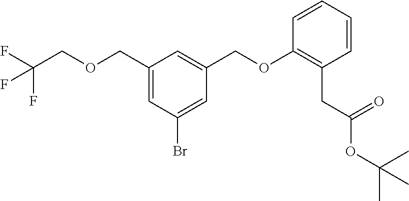 [SMILES: CC(C)(C)OC(=O)Cc1ccccc1OCc1cc(Br)cc(COCC(F)(F)F)c1]

To a solution of tert-butyl 2-(2-((3-bromo-5-(hydroxymethyl)benzyl)oxy)phenyl)acetate (Intermediate 59-A) (0.400 g, 0.982 mmol) and 1,1′-(azodicarbonyl)dipiperidine (0.496 g, 1.96 mmol) in toluene (9.8 ml) at room temperature under nitrogen was added tri-n-butylphosphine (0.485 ml, 1.96 mmol). After 10 minutes, trifluoroethanol (0.567 ml, 7.86 mmol) was added and this was stirred at room temperature. After 10 minutes additional toluene (9.8 ml) was added. After 45 minutes the reaction was concentrated and purified by flash chromatography (0-40% EtOAc:Heptanes) to provide the title compound. ¹H NMR (400 MHz, DMSO-d₆) δ ppm 7.60 (s, 1H) 7.50 (s, 1H) 7.41 (s, 1H) 7.15-7.28 (m, 2H) 7.01 (d, J=7.45 Hz, 1H) 6.91 (td, J=7.39, 0.88 Hz, 1H) 5.13 (s, 2H) 4.67 (s, 2H) 4.12 (q, J=9.35 Hz, 2H) 3.55 (s, 2H) 1.33 (s, 9H).

### Intermediate 108

### Intermediate 108-A

### (3-bromo-5-(((tert-butyldimethylsilyl)oxy)methyl)phenyl)methanol

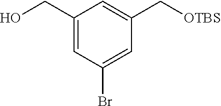 [SMILES: CC(C)(C)[Si](C)(C)OCc1cc(Br)cc(CO)c1]

To (5-bromo-1,3-phenylene)dimethanol (CAS #51760-22-6) (10.0 g, 46.1 mmol) in DCM (598 ml) at room temperature under nitrogen was added imidazole (3.76 g, 55.3 mmol) followed by TBSCl (7.64 g, 50.7 mmol). After 40 minutes the reaction was diluted with water and brine. The organic layer was washed with water, dried with MgSO₄ and concentrated. This was purified by flash chromatography (0-60% EtOAc:Heptanes) to provide the title compound. ¹H NMR (400 MHz, DMSO-d₆) δ ppm 7.37 (s, 1H) 7.34 (s, 1H) 7.21-7.28 (m, 1H) 5.29 (t, J=5.81 Hz, 1H) 4.70 (s, 2H) 4.48 (d, J=5.56 Hz, 2H) 0.90 (s, 9H) 0.08 (s, 6H).

### Intermediate 108-B

### ((3-bromo-5-(bromomethyl)benzyl)oxy)(tert-butyl)dimethylsilane

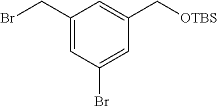 [SMILES: CC(C)(C)[Si](C)(C)OCc1cc(Br)cc(CBr)c1]

To (3-bromo-5-(((tert-butyldimethylsilyl)oxy)methyl)phenyl)methanol (3.10 g, 9.36 mmol) in DCM (94 ml) under nitrogen was added PPh₃ (3.68 g, 14.0 mmol) and CBr₄ (4.65 g, 14.0 mmol). After 5 minutes the reaction was partially concentrated and then purified directly by flash chromatography (0-15% EtOAc:Heptanes) to provide the title compound. ¹H NMR (400 MHz, DMSO-d₆) δ ppm 7.51-7.59 (m, 1H) 7.42 (s, 1H) 7.39 (s, 1H) 4.71 (s, 2H) 4.69 (s, 2H) 0.91 (s, 9H) 0.08 (s, 6H).

### Intermediate 108-C

### ((3-bromo-5-(isopropoxymethyl)benzyl)oxy)(tert-butyl)dimethylsilane

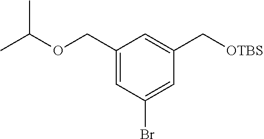 [SMILES: CC(C)OCc1cc(Br)cc(CO[Si](C)(C)C(C)(C)C)c1]

To a solution of 2-propanol (0.586 ml, 7.61 mmol) in DMF (25.4 ml) at 0° C. under nitrogen was added NaH (60% in mineral oil, 0.203 g, 5.07 mmol) and this was stirred for 20 minutes. A solution of ((3-bromo-5-(bromomethyl)benzyl)oxy)(tert-butyl)dimethylsilane (1.0 g, 2.54 mmol) in DMF (5 mL) was added and the reaction was stirred at room temperature. After 50 minutes the reaction was quenched with sat. aq. NH₄Cl, extracted with EtOAc, diluted with Heptanes, washed with water 2×, dried with MgSO₄, filtered and concentrated. This was purified by flash chromatography (0-20% EtOAc:Heptanes) to provide the title compound. ¹H NMR (400 MHz, DMSO-d₆) δ 7.40-7.33 (m, 2H), 7.31-7.24 (m, 1H), 4.77-4.64 (m, 2H), 4.45 (d, J=0.9 Hz, 2H), 3.63 (hept, J=6.1 Hz, 1H), 1.14 (d, J=6.1 Hz, 6H), 1.01-0.81 (m, 9H), 0.15-0.03 (m, 6H).

### Intermediate 108-D

### (3-bromo-5-(isopropoxymethyl)phenyl)methanol

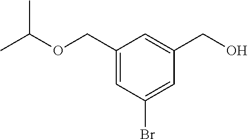

To a solution of ((3-bromo-5-(isopropoxymethyl)benzyl)oxy)(tert-butyl)dimethylsilane (0.45 g, 1.20 mmol) in THF (12.0 ml) at room temperature under nitrogen was added TBAF (1.0M in THF, 1.45 ml, 1.45 mmol). After 10 minutes the reaction was quenched with sat. aq. NH₄Cl, extracted with EtOAc 2×, dried with MgSO₄, filtered and concentrated. This was purified by flash chromatography (0-70% EtOAc:Heptanes) to provide the title compound. ¹H NMR (400 MHz, DMSO-d₆) δ ppm 7.39 (s, 1H) 7.35 (s, 1H) 7.20-7.28 (m, 1H) 5.30 (t, J=5.87 Hz, 1H) 4.48 (d, J=5.81 Hz, 2H) 4.44 (s, 2H) 3.64 (hept, J=6.11 Hz, 1H) 1.14 (d, J=6.06 Hz, 6H).

### Intermediate 108-E

### tert-butyl 2-(2-((3-bromo-5-(isopropoxymethyl)benzyl)oxy)phenyl)acetate

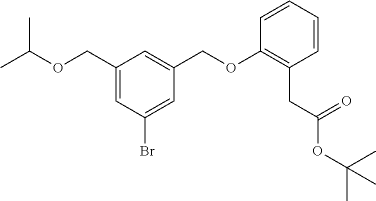 [SMILES: CC(C)OCc1cc(Br)cc(COc2ccccc2CC(=O)OC(C)(C)C)c1]

The title compound was synthesized as described in Intermediate 55 starting with (3-bromo-5-(isopropoxymethyl)phenyl)methanol. ¹H NMR (400 MHz, DMSO-d₆) δ ppm 7.54 (s, 1H) 7.45 (s, 1H) 7.38 (s, 1H) 7.17-7.27 (m, 2H) 7.01 (d, J=7.58 Hz, 1H) 6.91 (td, J=7.39, 1.01 Hz, 1H) 5.11 (s, 2H) 4.46 (s, 2H) 3.64 (hept, J=6.11 Hz, 1H) 3.54 (s, 2H) 1.33 (s, 9H) 1.14 (d, J=6.06 Hz, 6H).

### Intermediate 109

### Intermediate 109-A

### (3-bromo-5-(ethoxymethyl)phenyl)methanol

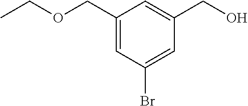

To (5-bromo-1,3-phenylene)dimethanol (1.0 g, 4.61 mmol) in THF (46.1 ml) at 0° C. under nitrogen was added NaH (60% in mineral oil, 0.295 g, 7.37 mmol) followed by Etl (0.596 ml, 7.37 mmol) and this was stirred at room temperature. After overnight additional NaH (60% in mineral oil, 0.295 g, 7.37 mmol) and Etl (0.596 ml, 7.37 mmol) were added and this was stirred under nitrogen at room temperature for 3 days. The reaction was diluted with water and brine, then extracted with EtOAc 2×. The organic layers were washed with water, dried with MgSO₄ and concentrated. This was purified by flash chromatography (0-60% EtOAc:Heptanes) to provide the title compound. ¹H NMR (400 MHz, DMSO-d₆) δ ppm 7.40 (s, 1H) 7.35 (s, 1H) 7.22-7.29 (m, 1H) 5.31 (t, J=5.87 Hz, 1H) 4.49 (d, J=5.56 Hz, 2H) 4.44 (s, 2H) 3.48 (q, J=6.99 Hz, 2H) 1.15 (t, J=6.95 Hz, 3H).

### Intermediate 109-B

### Tert-butyl 2-(2-((3-bromo-5-(ethoxymethyl)benzyl)oxy)phenyl)acetate

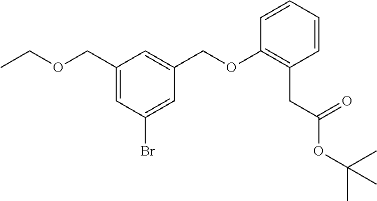 [SMILES: CCOCc1cc(Br)cc(COc2ccccc2CC(=O)OC(C)(C)C)c1]

The title compound was synthesized as described in Intermediate 55 starting with (3-bromo-5-(ethoxymethyl)phenyl)methanol. ¹H NMR (400 MHz, DMSO-d₆) δ ppm 7.55 (s, 1H) 7.46 (s, 1H) 7.38 (s, 1H) 7.16-7.27 (m, 2H) 7.01 (d, J=7.58 Hz, 1H) 6.91 (td, J=7.39, 1.01 Hz, 1H) 5.12 (s, 2H) 4.46 (s, 2H) 3.54 (s, 2H) 3.49 (q, J=6.99 Hz, 2H) 1.33 (s, 9H) 1.16 (t, J=7.01 Hz, 3H).

### Intermediate 110

### Intermediate 110-A

### ((3-bromo-5-(((6-chlorochroman-4-yl)oxy)methyl)benzyl)oxy)(tert-butyl)dimethylsilane

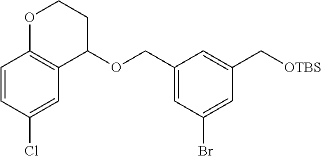 [SMILES: CC(C)(C)[Si](C)(C)OCc1cc(Br)cc(COC2CCOc3ccc(Cl)cc32)c1]

To a solution of 6-chlorochroman-4-ol (CAS #18385-76-7) (0.955 g, 5.17 mmol) in DMF (25.9 ml) at 0° C. under nitrogen was added NaH (60% in mineral oil, 0.155 g, 3.88 mmol) and this was stirred for 20 minutes. A solution of ((3-bromo-5-(bromomethyl)benzyl)oxy)(tert-butyl)dimethylsilane (Intermediate 108-B) (1.02 g, 2.59 mmol) in DMF (5 mL) was added and the reaction was stirred at room temperature. After 40 minutes the reaction was quenched with sat. aq. NH₄Cl, extracted with EtOAc, diluted with Heptanes, washed with water 2×, dried with MgSO₄, filtered and concentrated. This was purified by flash chromatography (0-20% EtOAc:Heptanes) to provide the title compound. ¹H NMR (400 MHz, DMSO-d₆) δ ppm 7.44 (s, 1H) 7.40 (s, 1H) 7.34 (s, 1H) 7.30 (d, J=2.65 Hz, 1H) 7.20-7.26 (m, 1H) 6.77-6.86 (m, 1H) 4.65-4.77 (m, 3H) 4.56-4.65 (m, 1H) 4.54 (t, J=3.92 Hz, 1H) 4.25 (dt, J=10.89, 4.09 Hz, 1H) 4.16 (td, J=10.86, 2.78 Hz, 1H) 2.08-2.20 (m, 1H) 1.91-2.08 (m, 1H) 0.89 (s, 9H) 0.07 (s, 6H).

### Intermediate 110-B

### (3-bromo-5-(((6-chlorochroman-4-yl)oxy)methyl)phenyl)methanol

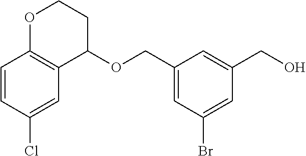 [SMILES: OCc1cc(Br)cc(COC2CCOc3ccc(Cl)cc32)c1]

The title compound was synthesized as described in Intermediate 108-D starting with ((3-bromo-5-(((6-chlorochroman-4-yl)oxy)methyl)benzyl)oxy)(tert-butyl)dimethylsilane. ¹H NMR (400 MHz, DMSO-d₆) δ ppm 7.42 (s, 2H) 7.29-7.35 (m, 2H) 7.23 (dd, J=8.78, 2.72 Hz, 1H) 6.83 (d, J=8.72 Hz, 1H) 5.32 (t, J=5.75 Hz, 1H) 4.57-4.74 (m, 2H) 4.54 (t, J=3.98 Hz, 1H) 4.50 (d, J=5.43 Hz, 2H) 4.22-4.31 (m, 1H) 4.11-4.22 (m, 1H) 2.09-2.19 (m, 1H) 1.97-2.07 (m, 1H).

### Intermediate 110-C

### Tert-butyl 2-(2-((3-bromo-5-(((6-chlorochroman-4-yl)oxy)methyl)benzyl)oxy)phenyl)acetate

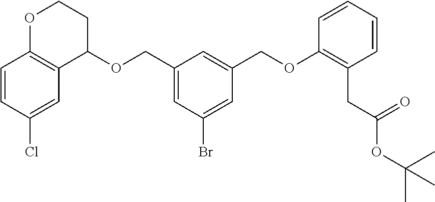 [SMILES: CC(C)(C)OC(=O)Cc1ccccc1OCc1cc(Br)cc(COC2CCOc3ccc(Cl)cc32)c1]

The title compound was synthesized as described in Intermediate 55 starting with (3-bromo-5-(((6-chlorochroman-4-yl)oxy)methyl)phenyl)methanol. ¹H NMR (400 MHz, DMSO-d₆) δ ppm 7.57 (s, 1H) 7.53 (s, 1H) 7.44 (s, 1H) 7.32 (d, J=2.65 Hz, 1H) 7.16-7.28 (m, 3H) 7.01 (d, J=7.71 Hz, 1H) 6.90 (td, J=7.39, 1.01 Hz, 1H) 6.83 (d, J=8.84 Hz, 1H) 5.12 (s, 2H) 4.72 (d, J=12.13 Hz, 1H) 4.60 (d, J=12.13 Hz, 1H) 4.55 (t, J=3.92 Hz, 1H) 4.13-4.29 (m, 2H) 3.54 (s, 2H) 2.07-2.20 (m, 1H) 1.94-2.07 (m, 1H) 1.31 (s, 9H).

### Intermediate 111

### Intermediate 111-A

### ((3-bromo-5-chlorobenzyl)oxy)(tert-butyl)dimethylsilane

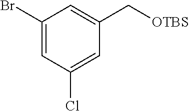 [SMILES: CC(C)(C)[Si](C)(C)OCc1cc(Cl)cc(Br)c1]

The title compound was synthesized as described in Intermediate 108-A starting with (3-bromo-5-chlorophenyl)methanol (CAS #917562-09-5). ¹H NMR (400 MHz, DMSO-d₆) δ ppm 7.59 (t, J=1.83 Hz, 1H) 7.45-7.50 (m, 1H) 7.36 (dd, J=1.89, 1.26 Hz, 1H) 4.72 (s, 2H) 0.90 (s, 9H) 0.08 (s, 6H).

### Intermediate 111-B

### Tert-butyl((3-chloro-5-vinylbenzyl)oxy)dimethylsilane

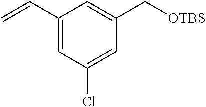 [SMILES: C=Cc1cc(Cl)cc(CO[Si](C)(C)C(C)(C)C)c1]

The title compound was synthesized as described in Intermediate 14-B starting with ((3-bromo-5-chlorobenzyl)oxy)(tert-butyl)dimethylsilane. ¹H NMR (400 MHz, DMSO-d₆) δ ppm 7.42-7.46 (m, 1H) 7.35 (s, 1H) 7.24 (s, 1H) 6.72 (dd, J=17.68, 10.86 Hz, 1H) 5.84-5.94 (m, 1H) 5.34 (d, J=11.37 Hz, 1H) 4.71 (s, 2H) 0.91 (s, 9H) 0.09 (s, 6H).

### Intermediate 111-C

### 2-(3-(((tert-butyldimethylsilyl)oxy)methyl)-5-chlorophenyl)ethanol

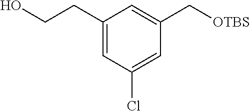 [SMILES: CC(C)(C)[Si](C)(C)OCc1cc(Cl)cc(CCO)c1]

A solution of tert-butyl((3-chloro-5-vinylbenzyl)oxy)dimethylsilane (0.88 g, 3.11 mmol) in THF (31.1 ml) was cooled to 0° C. under nitrogen and 9-BBN (0.5M in THF, 18.7 ml, 9.33 mmol) was added dropwise. This was warmed to room temperature and stirred overnight. This was cooled again to 0° C. and aq. NaOH (2.0M, 9.33 ml, 18.7 mmol) and H₂O₂ (50% aq., 1.14 ml, 18.7 mmol) were added, then this was warmed again to room temperature. After 15 minutes this was diluted with sat. aq. sodium thiosulfate and EtOAc, extracted 2× with EtOAc, dried with MgSO₄, filtered and concentrated. This was purified by flash chromatography (0-50% EtOAc:Heptanes) to provide the title compound. ¹H NMR (400 MHz, DMSO-d₆) δ ppm 7.16 (s, 2H) 7.11 (s, 1H) 4.68 (s, 2H) 4.64 (t, J=5.18 Hz, 1H) 3.59 (td, J=6.76, 5.18 Hz, 2H) 2.71 (t, J=6.82 Hz, 2H) 0.90 (s, 9H) 0.08 (s, 6H).

### Intermediate 111-D

### Tert-butyl((3-chloro-5-(2-methoxyethyl)benzyl)oxy)dimethylsilane

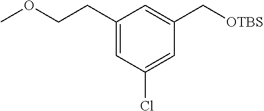 [SMILES: COCCc1cc(Cl)cc(CO[Si](C)(C)C(C)(C)C)c1]

To a solution of 2-(3-(((tert-butyldimethylsilyl)oxy)methyl)-5-chlorophenyl)ethanol (0.390 g, 1.30 mmol) in THF (13.0 ml) at 0° C. was added NaH (60% in mineral oil, 0.073 g, 1.81 mmol) and then Mel (0.113 ml, 1.81 mmol) and this was stirred at room temperature. After overnight additional NaH (60% in mineral oil, 0.036 g, 0.90 mmol) and Mel (0.056 ml, 0.90 mmol) were added. After 5 more hours additional NaH (60% in mineral oil, 0.036 g, 0.90 mmol) and Mel (0.056 ml, 0.90 mmol) were added and this was stirred overnight again. The reaction was quenched with sat. aq. NH₄Cl, extracted with EtOAc, dried with MgSO₄, filtered and concentrated. This was purified by flash chromatography (0-50% EtOAc:Heptanes) to provide the title compound. ¹H NMR (400 MHz, DMSO-d₆) δ 7.21-7.15 (m, 2H), 7.13 (s, 1H), 4.68 (s, 2H), 3.53 (t, J=6.6 Hz, 2H), 3.22 (s, 3H), 2.80 (t, J=6.6 Hz, 2H), 0.90 (s, 9H), 0.08 (s, 6H).

### Intermediate 111-E

### (3-chloro-5-(2-methoxyethyl)phenyl)methanol

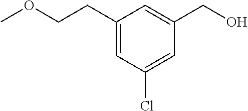

The title compound was synthesized as described in Intermediate 108-D starting with tert-butyl((3-chloro-5-(2-methoxyethyl)benzyl)oxy)dimethylsilane. ¹H NMR (400 MHz, DMSO-d₆) δ 7.22-7.14 (m, 2H), 7.12 (s, 1H), 5.27 (t, J=5.7 Hz, 1H), 4.46 (dd, J=5.5, 0.9 Hz, 2H), 3.53 (t, J=6.7 Hz, 2H), 3.23 (s, 3H), 2.80 (t, J=6.6 Hz, 2H).

### Intermediate 111-F

### Tert-butyl 2-(2-((3-chloro-5-(2-methoxyethyl)benzyl)oxy)phenyl)acetate

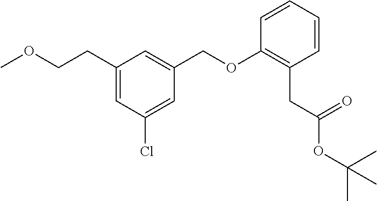 [SMILES: COCCc1cc(Cl)cc(COc2ccccc2CC(=O)OC(C)(C)C)c1]

The title compound was synthesized as described in Intermediate 55 starting with (3-chloro-5-(2-methoxyethyl)phenyl)methanol. ¹H NMR (400 MHz, DMSO-d₆) δ 7.34 (t, J=1.7 Hz, 1H), 7.29-7.16 (m, 4H), 7.01 (dd, J=8.2, 1.1 Hz, 1H), 6.91 (td, J=7.4, 1.1 Hz, 1H), 5.09 (s, 2H), 3.59-3.50 (m, 4H), 3.23 (s, 3H), 2.82 (t, J=6.6 Hz, 2H), 1.33 (s, 9H).

### Intermediate 112

### Intermediate 112-A. (3-bromo-5-((difluoromethoxy)methyl)phenyl)methanol

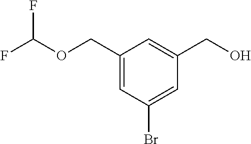 [SMILES: OCc1cc(Br)cc(COC(F)F)c1]

To (5-bromo-1,3-phenylene)dimethanol (2.10 g, 9.66 mmol) in Acetonitrile (38.6 ml) at room temperature under nitrogen was added Na₂SO₄ (0.274 g, 1.93 mmol). This was heated to 50° C. and 2,2-difluoro-2-(fluorosulfonyl)acetic acid (0.998 ml, 9.66 mmol) was added. After 2 hours the reaction was cooled, diluted with sat. aq. sodium bicarbonate, extracted with EtOAc, dried with MgSO₄ and concentrated. This was purified by flash chromatography (0-70% EtOAc:Heptanes) to give a mixture containing the title compound. This was taken up in MeOH (40 mL) and NaOH (0.773 g, 19.3 mmol) was added. After stirring at room temperature overnight the reaction was diluted with sat. aq. NH₄Cl and water, extracted with EtOAc, dried with MgSO₄ and concentrated. This was purified by flash chromatography (0-70% EtOAc:Heptanes) to provide the title compound. ¹H NMR (400 MHz, DMSO-d₆) δ 7.50-7.46 (m, 1H), 7.44 (ddt, J=2.0, 1.3, 0.7 Hz, 1H), 7.33 (tt, J=1.4, 0.8 Hz, 1H), 6.80 (t, J=75.5 Hz, 1H), 5.35 (t, J=5.7 Hz, 1H), 4.91 (s, 2H), 4.51 (dd, J=5.6, 0.9 Hz, 2H).

### Intermediate 112-B

### Tert-butyl 2-(2-((3-bromo-5-((difluoromethoxy)methyl)benzyl)oxy)phenyl)acetate

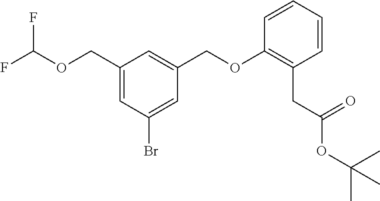 [SMILES: CC(C)(C)OC(=O)Cc1ccccc1OCc1cc(Br)cc(COC(F)F)c1]

The title compound was synthesized as described in Intermediate 55 starting with (3-bromo-5-((difluoromethoxy)methyl)phenyl)methanol. ¹H NMR (400 MHz, DMSO-d₆) δ 7.63 (t, J=1.6 Hz, 1H), 7.55 (t, J=1.6 Hz, 1H), 7.45 (dd, J=2.0, 1.2 Hz, 1H), 7.29-7.16 (m, 2H), 7.02 (dd, J=8.2, 1.1 Hz, 1H), 6.91 (d, J=1.1 Hz, 1H), 6.81 (t, J=75.4 Hz, 1H), 5.13 (s, 2H), 4.93 (s, 2H), 3.55 (s, 2H), 1.33 (s, 9H).

### Intermediate 113

### Intermediate 113-A

### Methyl 3-bromo-5-((tert-butyldimethylsilyl)oxy)benzoate

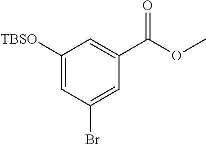 [SMILES: COC(=O)c1cc(Br)cc(O[Si](C)(C)C(C)(C)C)c1]

TBSCl (4.31 g, 28.6 mmol) was added to a solution of methyl 3-bromo-5-hydroxybenzoate (6 g, 26.0 mmol) and imidazole (2.30 g, 33.8 mmol) in DMF (20 mL) at 23° C. The reaction mixture was stirred at rt overnight, then partitioned between 1:1 EtOAc/heptane and water. The aqueous layer was extracted with 1:1 EtOAc/heptane. The combined organics were washed with brine, dried (Na₂SO₄) and concentrated to provide crude product, which was used in next reaction without further purification.

### Intermediate 113-B

### (3-Bromo-5-((tert-butyldimethylsilyl)oxy)phenyl)methanol

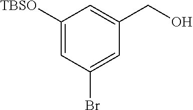 [SMILES: CC(C)(C)[Si](C)(C)Oc1cc(Br)cc(CO)c1]

LiBH₄ (2.83 g, 130 mmol) was added to a solution of methyl 3-bromo-5-((tert-butyldimethylsilyl)oxy)benzoate (8.98 g, 26 mmol) in THF (100 mL) at −78° C., followed by MeOH (5.26 mL, 130 mmol). The resulting mixture was allowed to warm to rt and stirred overnight. The mixture was cooled to 0° C. and sat. NH₄Cl was added slowly to quench excess LiBH₄. The mixture was then extracted with EtOAc (3×). The combined organics were washed with brine, dried (Na₂SO₄) and concentrated. The residue was purified by silica gel chromatography (EtOAc-heptane 0-50%) to provide the desired product. ¹H NMR (400 MHz, CD₂Cl₂) δ ppm 7.15 (ddt, J=2.0, 1.4, 0.7 Hz, 1H), 6.97 (td, J=2.0, 0.6 Hz, 1H), 6.82 (ddt, J=2.2, 1.4, 0.7 Hz, 1H), 4.63 (q, J=0.6 Hz, 2H), 1.03 (s, 10H), 0.25 (s, 6H).

### Intermediate 113-C

### Tert-butyl 2-(2-((3-bromo-5-((tert-butyldimethylsilyl)oxy)benzyl)oxy)phenyl)acetate

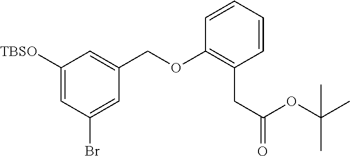 [SMILES: CC(C)(C)OC(=O)Cc1ccccc1OCc1cc(Br)cc(O[Si](C)(C)C(C)(C)C)c1]

DIAD (5.49 mL, 28.2 mmol) was added dropwise to a solution of (3-bromo-5-((tert-butyldimethylsilyl)oxy)phenyl)methanol (7.46 g, 23.51 mmol), tert-butyl 2-(2-hydroxyphenyl)acetate (Intermediate 21) (5.88 g, 28.2 mmol) and PPh₃ (7.40 g, 28.2 mmol) in THF (100 mL) at 0° C. The resulting yellow solution was allowed to warm to room temperature and then stirred for 2 hr. NH₄Cl was added and the mixture was extracted with EtOAc (2×). The combined organics were washed with brine, dried (Na₂SO₄) and concentrated. The residue was purified by ISCO (EtOAc-heptane 0-50%) to provide a mixture of the title compound and the free phenol product where the TBS was removed. This mixture was used in the next step without further purification.

### Intermediate 113-D

### Tert-butyl 2-(2-((3-bromo-5-hydroxybenzyl)oxy)phenyl)acetate

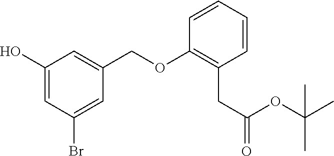 [SMILES: CC(C)(C)OC(=O)Cc1ccccc1OCc1cc(O)cc(Br)c1]

TBAF (14.38 mL, 14.38 mmol) was added to a mixture of tert-butyl 2-(2-((3-bromo-5-((tert-butyldimethylsilyl)oxy)benzyl)oxy)phenyl)acetate and the corresponding free phenol (7.30 g, 14.38 mmol) in THF (100 mL) at 0° C. The reaction mixture was stirred at 0° C. for 1.5 hr. Sat. aqueous NH₄Cl was added and the layers were separated. The aqueous layer was extracted with EtOAc (2×). The combined organics were washed with brine, dried (Na₂SO₄), and concentrated. The residue was purified by ISCO (EtOAc-heptane 0-30%) to provide the desired product as a white solid. ¹H NMR (400 MHz, CD₃OD) δ ppm 7.27-7.12 (m, 2H), 7.11-6.80 (m, 5H), 4.97 (s, 2H), 3.57 (s, 2H), 1.37 (s, 9H).

### Intermediate 114

### Intermediate 114-A

### (R)-tert-Butyl 2-(2-((3′-(1-((tert-butoxycarbonyl)amino)ethyl)-2′-fluoro-5-hydroxy-[1,1′-biphenyl]-3-yl)methoxy)phenyl)acetate

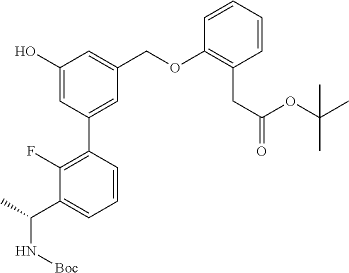 [SMILES: C[C@@H](NC(=O)OC(C)(C)C)c1cccc(-c2cc(O)cc(COc3ccccc3CC(=O)OC(C)(C)C)c2)c1F]

A degassed mixture of tert-butyl 2-(2-((3-bromo-5-hydroxybenzyl)oxy)phenyl)acetate (Intermediate 113-D) (1300 mg, 3.31 mmol), (R)-tert-butyl 2-(2-((3′-(1-((tert-butoxycarbonyl)amino)ethyl)-2′-fluoro-5-hydroxy-[1,1′-biphenyl]-3-yl)methoxy)phenyl)acetate (intermediate 27B) (1328 mg, 3.64 mmol), PdCl₂(dppf).CH₂Cl₂ adduct (CAS #95464-05-4) (135 mg, 0.165 mmol), acetonitrile (10 ml), and K₃PO₄ (2 M aqueous solution, 4.96 ml, 9.92 mmol) in a sealed vial was heated in a microwave at 100° C. for 60 min. The reaction mixture was cooled to rt, the organic layer was filtered and purified by flash column (EtOAc/Heptane 0-100%) to give the title compound. MS (ESI−) m/z 550.4 (M−H).

### Intermediate 114-B

### (S)-tert-Butyl 2-(2-((3′-(1-((tert-butoxycarbonyl)amino)ethyl)-2′-fluoro-5-hydroxy-[1,1′-biphenyl]-3-yl)methoxy)phenyl)acetate

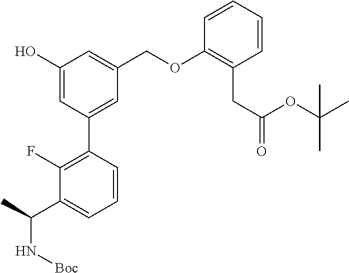 [SMILES: C[C@H](NC(=O)OC(C)(C)C)c1cccc(-c2cc(O)cc(COc3ccccc3CC(=O)OC(C)(C)C)c2)c1F]

The title compound was synthesized in a similar manner as described in Intermediate X-A starting with tert-butyl 2-(2-((3-bromo-5-hydroxybenzyl)oxy)phenyl)acetate (Intermediate #-D) and (S)-tert-butyl (1-(2-fluoro-3-(4,4,5,5-tetramethyl-1,3,2-dioxaborolan-2-yl)phenyl)ethyl)carbamate (Intermediate 105). MS (ESI−) m/z 550.4 (M−H).

### Intermediate 115

### (R)-tert-Butyl 2-(2-((3′-(1-((tert-butoxycarbonyl)amino)ethyl)-2′-fluoro-5-(pyridin-2-ylmethoxy)-[1,1′-biphenyl]-3-yl)methoxy)phenyl)acetate

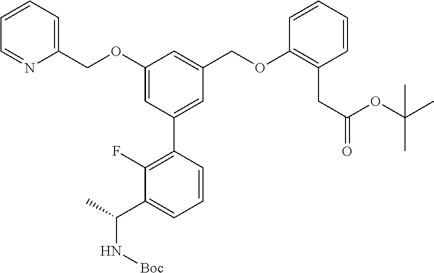 [SMILES: C[C@@H](NC(=O)OC(C)(C)C)c1cccc(-c2cc(COc3ccccc3CC(=O)OC(C)(C)C)cc(OCc3ccccn3)c2)c1F]

A solution of (R)-tert-butyl 2-(2-((3′-(1-((tert-butoxycarbonyl)amino)ethyl)-2′-fluoro-5-hydroxy-[1,1′-biphenyl]-3-yl)methoxy)phenyl)acetate (Intermediate 114-A) (50 mg, 0.091 mmol), pyridin-2-ylmethanol (19.78 mg, 0.181 mmol) and PPh₃ (30.9 mg, 0.118 mmol) in THF (3 mL) was cooled to 0° C. in an ice/water bath under nitrogen. DIAD (0.023 mL, 0.118 mmol) was added dropwise. The resulting yellow solution was allowed to warm to room temperature and then stirred at room temperature for 16 hours. The mixture was diluted with water and EtOAc, extracted with EtOAc, washed with brine, dried with MgSO₄, filtered and concentrated. The residue was purified by flash chromatography (EtOAc/heptane 0-100%) to provide the title compound. MS (ESI+) m/z 643.5 (M+H).

### Intermediate 116

The following compounds were prepared with similar methods as described in Intermediate 115 using the appropriate phenol and alcohol as starting materials.

| | | | | MS (ESI+) |
|---|---|---|---|---|
| | Structure/Chemical Name | Phenol | alcohol | m/z |
| 116-A | 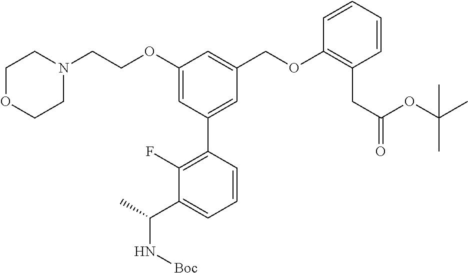 [SMILES: C[C@@H](NC(=O)OC(C)(C)C)c1cccc(-c2cc(COc3ccccc3CC(=O)OC(C)(C)C)cc(OCCN3CCOCC3)c2)c1F] | Interme diate 114-A | 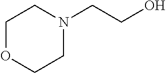 2- morpholinoethanol | 665.6 (M + H) |
| | (R)-tert-butyl 2-(2-((3′-(1-((tert- | | | |
| | butoxycarbonyl)amino)ethyl)-2′- | | | |
| | fluoro-5-(2-morpholinoethoxy)-[1,1′- | | | |
| | biphenyl]-3- | | | |
| | yl)methoxy)phenyl)acetate | | | |
| 116-B | 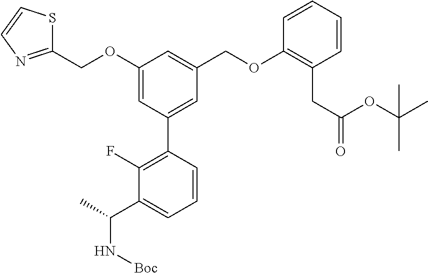 [SMILES: C[C@@H](NC(=O)OC(C)(C)C)c1cccc(-c2cc(COc3ccccc3CC(=O)OC(C)(C)C)cc(OCc3nccs3)c2)c1F] | Interme diate 114-A | 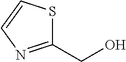 thiazol-2- ylmethanol | 649.5 (M + H) |
| | (R)-tert-butyl 2-(2-((3′-(1-((tert- | | | |
| | butoxycarbonyl)amino)ethyl)-2′- | | | |
| | fluoro-5-(thiazol-2-ylmethoxy)-[1,1′- | | | |
| | biphenyl]-3- | | | |
| | yl)methoxy)phenyl)acetate | | | |
| 116-C | 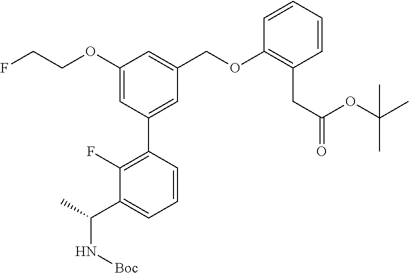 [SMILES: C[C@@H](NC(=O)OC(C)(C)C)c1cccc(-c2cc(COc3ccccc3CC(=O)OC(C)(C)C)cc(OCCF)c2)c1F] | Interme diate 114-A | 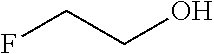 2-fluoroethanol | 598.4 (M + H) |
| | (R)-tert-butyl 2-(2-((3′-(1-((tert- | | | |
| | butoxycarbonyl)amino)ethyl)-2′- | | | |
| | fluoro-5-(2-fluoroethoxy)-[1,1′- | | | |
| | biphenyl]-3- | | | |
| | yl)methoxy)phenyl)acetate | | | |
| 116-D | 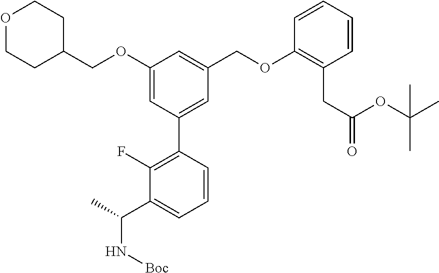 [SMILES: C[C@@H](NC(=O)OC(C)(C)C)c1cccc(-c2cc(COc3ccccc3CC(=O)OC(C)(C)C)cc(OCC3CCOCC3)c2)c1F] | Interme diate 114-A | 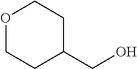 (tetrahydro-2H- pyran-4- yl)methanol | crude used on next step without purification. |
| | (R)-tert-butyl 2-(2-((3′-(1-((tert- | | | |
| | butoxycarbonyl)amino)ethyl)-2′- | | | |
| | fluoro-5-((tetrahydro-2H-pyran-4- | | | |
| | yl)methoxy)-[1,1′-biphenyl]-3- | | | |
| | yl)methoxy)phenyl)acetate | | | |
| 116-E | 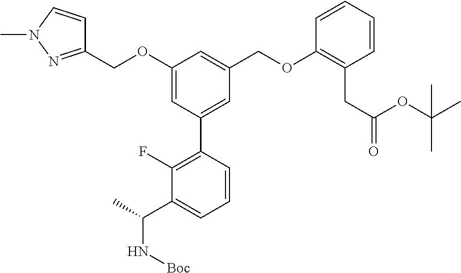 [SMILES: C[C@@H](NC(=O)OC(C)(C)C)c1cccc(-c2cc(COc3ccccc3CC(=O)OC(C)(C)C)cc(OCc3ccn(C)n3)c2)c1F] | Interme diate 114-A | 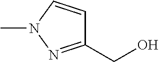 (1-methyl-1H- pyrazol-3- yl)methanol | 646.6 (M + H) |
| | (R)-tert-butyl 2-(2-((3′-(1-((tert- | | | |
| | butoxycarbonyl)amino)ethyl)-2′- | | | |
| | fluoro-5-((1-methyl-1H-pyrazol-3- | | | |
| | yl)methoxy)-[1,1′-biphenyl]-3- | | | |
| | yl)methoxy)phenyl)acetate | | | |
| 116-F | 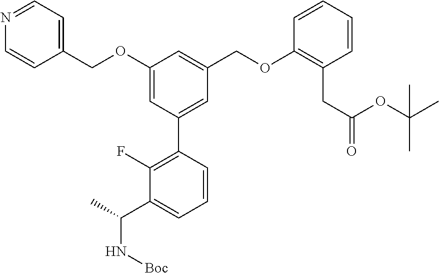 [SMILES: C[C@@H](NC(=O)OC(C)(C)C)c1cccc(-c2cc(COc3ccccc3CC(=O)OC(C)(C)C)cc(OCc3ccncc3)c2)c1F] | Interme diate 114-A | 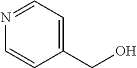 pyridin-4- ylmethanol | 643.6 (M + H) |
| | (R)-tert-butyl 2-(2-((3′-(1-((tert- | | | |
| | butoxycarbonyl)amino)ethyl)-2′- | | | |
| | fluoro-5-(pyridin-4-ylmethoxy)-[1,1′- | | | |
| | biphenyl]-3- | | | |
| | yl)methoxy)phenyl)acetate | | | |
| 116-G | 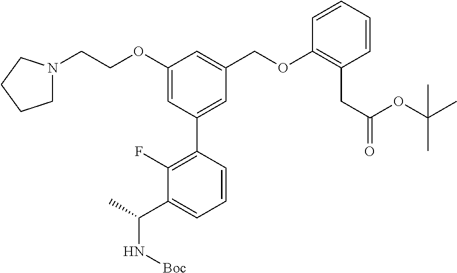 [SMILES: C[C@@H](NC(=O)OC(C)(C)C)c1cccc(-c2cc(COc3ccccc3CC(=O)OC(C)(C)C)cc(OCCN3CCCC3)c2)c1F] | Interme diate 114-A | 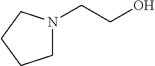 2-(pyrrolidin-1- yl)ethanol | 649.6 (M + H) |
| | (R)-tert-butyl 2-(2-((3′-(1-((tert- | | | |
| | butoxycarbonyl)amino)ethyl)-2′- | | | |
| | fluoro-5-(2-(pyrrolidin-1-yl)ethoxy)- | | | |
| | [1,1′-biphenyl]-3- | | | |
| | yl)methoxy)phenyl)acetate | | | |
| 116-H | 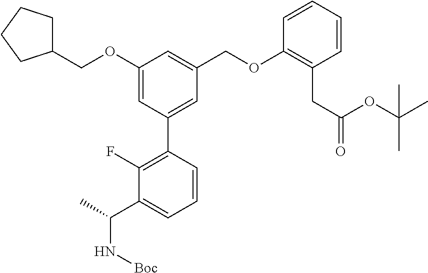 [SMILES: C[C@@H](NC(=O)OC(C)(C)C)c1cccc(-c2cc(COc3ccccc3CC(=O)OC(C)(C)C)cc(OCC3CCCC3)c2)c1F] | Interme diate 114-A | 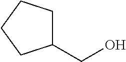 cyclopentylmethanol | 656.6 (M + Na) |
| | (R)-tert-butyl 2-(2-((3′-(1-((tert- | | | |
| | butoxycarbonyl)amino)ethyl)-5- | | | |
| | (cyclopentylmethoxy)-2′-fluoro- | | | |
| | [1,1′-biphenyl]-3- | | | |
| | yl)methoxy)phenyl)acetate | | | |
| 116-I | 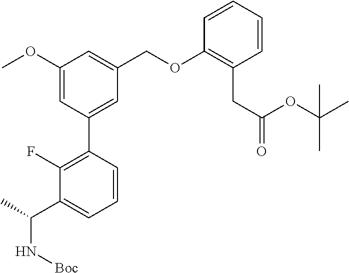 [SMILES: COc1cc(COc2ccccc2CC(=O)OC(C)(C)C)cc(-c2cccc([C@@H](C)NC(=O)OC(C)(C)C)c2F)c1] | Interme diate 114-A | methanol | 466.4 (M − Boc) |
| | (R)-tert-butyl 2-(2-((3′-(1-((tert- | | | |
| | butoxycarbonyl)amino)ethyl)-2′- | | | |
| | fluoro-5-methoxy-[1,1′-biphenyl]-3- | | | |
| | yl)methoxy)phenyl)acetate | | | |
| 116-J | 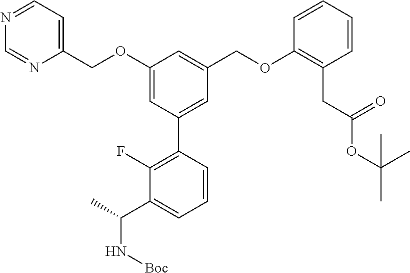 [SMILES: C[C@@H](NC(=O)OC(C)(C)C)c1cccc(-c2cc(COc3ccccc3CC(=O)OC(C)(C)C)cc(OCc3ccncn3)c2)c1F] | Interme diate 114-A | 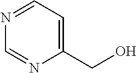 pyrimidin-4- ylmethanol | 666.5 (M + Na) |
| | (R)-tert-butyl 2-(2-((3′-(1-((tert- | | | |
| | butoxycarbonyl)amino)ethyl)-2′- | | | |
| | fluoro-5-(pyrimidin-4-ylmethoxy)- | | | |
| | [1,1′-biphenyl]-3- | | | |
| | yl)methoxy)phenyl)acetate | | | |
| 116-K | 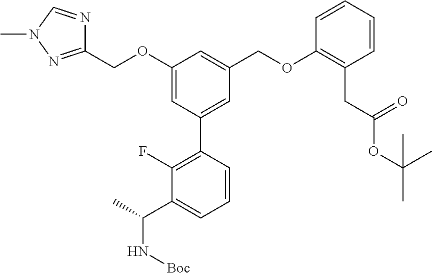 [SMILES: C[C@@H](NC(=O)OC(C)(C)C)c1cccc(-c2cc(COc3ccccc3CC(=O)OC(C)(C)C)cc(OCc3ncn(C)n3)c2)c1F] | Interme diate 114-A | 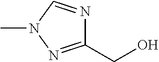 (1-methyl-1H-1,2,4- triazol-3- yl)methanol | 647.6 (M + H) |
| | (R)-tert-butyl 2-(2-((3′-(1-((tert- | | | |
| | butoxycarbonyl)amino)ethyl)-2′- | | | |
| | fluoro-5-((1-methyl-1H-1,2,4-triazol- | | | |
| | 3-yl)methoxy)-[1,1′-biphenyl]-3- | | | |
| | yl)methoxy)phenyl)acetate | | | |
| 116-L | 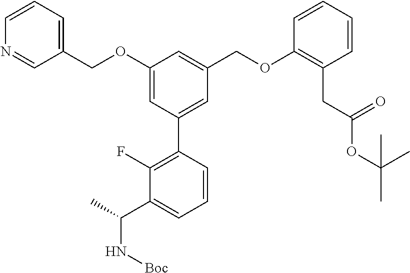 [SMILES: C[C@@H](NC(=O)OC(C)(C)C)c1cccc(-c2cc(COc3ccccc3CC(=O)OC(C)(C)C)cc(OCc3cccnc3)c2)c1F] | Interme diate 114-A | 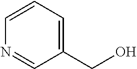 pyridin-3- ylmethanol | 643.6 (M + H) |
| | (R)-tert-butyl 2-(2-((3′-(1-((tert- | | | |
| | butoxycarbonyl)amino)ethyl)-2′- | | | |
| | fluoro-5-(pyridin-3-ylmethoxy)-[1,1′- | | | |
| | biphenyl]-3- | | | |
| | yl)methoxy)phenyl)acetate | | | |
| 116-M | 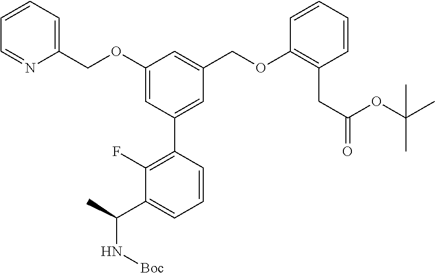 [SMILES: C[C@H](NC(=O)OC(C)(C)C)c1cccc(-c2cc(COc3ccccc3CC(=O)OC(C)(C)C)cc(OCc3ccccn3)c2)c1F] | Interme diate 114-B | 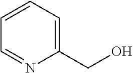 pyridin-2- ylmethanol | 643.5 (M + H) |
| | (S)-tert-butyl 2-(2-((3′-(1-((tert- | | | |
| | butoxycarbonyl)amino) ethyl)-2′- | | | |
| | fluoro-5-(pyridin-2-ylmethoxy)-[1,1′- | | | |
| | biphenyl]-3- | | | |
| | yl)methoxy)phenyl)acetate | | | |
| 116-N | 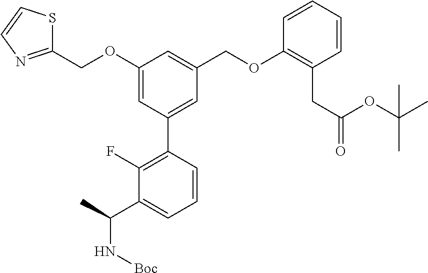 [SMILES: C[C@H](NC(=O)OC(C)(C)C)c1cccc(-c2cc(COc3ccccc3CC(=O)OC(C)(C)C)cc(OCc3nccs3)c2)c1F] | Interme diate 114-B | 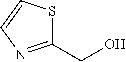 thiazol-2- ylmethanol | 649.5 (M + H) |
| | (S)-tert-butyl 2-(2-((3′-(1-((tert- | | | |
| | butoxycarbonyl)amino)ethyl)-2′- | | | |
| | fluoro-5-(thiazol-2-ylmethoxy)-[1,1′- | | | |
| | biphenyl]-3- | | | |
| | yl)methoxy)phenyl)acetate | | | |
| 116-O | 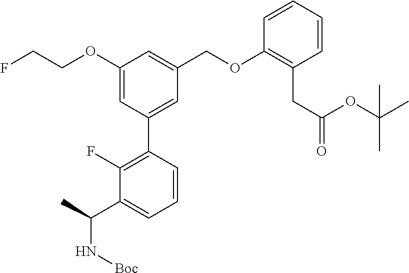 [SMILES: C[C@H](NC(=O)OC(C)(C)C)c1cccc(-c2cc(COc3ccccc3CC(=O)OC(C)(C)C)cc(OCCF)c2)c1F] | Interme diate 114-B | 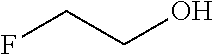 2-fluoroethanol | 598.4 (M + H) |
| | (S)-tert-butyl 2-(2-((3′-(1-((tert- | | | |
| | butoxycarbonyl)amino)ethyl)-2′- | | | |
| | fluoro-5-(2-fluoroethoxy)-[1,1′- | | | |
| | biphenyl]-3- | | | |
| | yl)methoxy)phenyl)acetate | | | |
| 116-P | 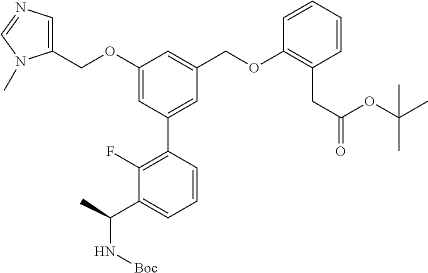 [SMILES: C[C@H](NC(=O)OC(C)(C)C)c1cccc(-c2cc(COc3ccccc3CC(=O)OC(C)(C)C)cc(OCc3cncn3C)c2)c1F] | Interme diate 114-B | 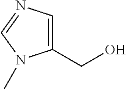 (1-methyl-1H- imidazol-5- yl)methanol | 646.6 (M + H) |
| | (S)-tert-butyl 2-(2-((3′-(1-((tert- | | | |
| | butoxycarbonyl)amino)ethyl)-2′- | | | |
| | fluoro-5-((1-methyl-1H-imidazol-5- | | | |
| | yl)methoxy)-[1,1′-biphenyl]-3- | | | |
| | yl)methoxy)phenyl)acetate | | | |
| 116-Q | 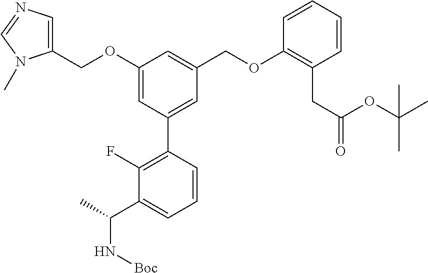 [SMILES: C[C@@H](NC(=O)OC(C)(C)C)c1cccc(-c2cc(COc3ccccc3CC(=O)OC(C)(C)C)cc(OCc3cncn3C)c2)c1F] | Interme diate 114-A | 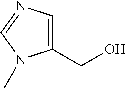 (1-methyl-1H- imidazol-5- yl)methanol | 646.6 (M + H) |
| | (R)-tert-butyl 2-(2-((3′-(1-((tert- | | | |
| | butoxycarbonyl)amino)ethyl)-2′- | | | |
| | fluoro-5-((1-methyl-1H-imidazol-5- | | | |
| | yl)methoxy)-[1,1′-biphenyl]-3- | | | |
| | yl)methoxy)phenyl)acetate | | | |

### Intermediate 117

### (R)-tert-butyl 2-(2-((3′-(1-((tert-butoxycarbonyl)amino)ethyl)-5-chloro-2′-fluoro-[1,1′-biphenyl]-3-yl)methoxy)phenyl)acetate

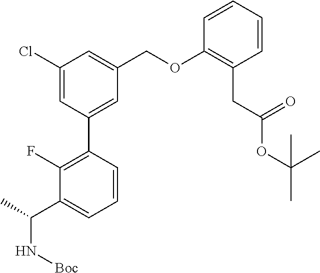 [SMILES: C[C@@H](NC(=O)OC(C)(C)C)c1cccc(-c2cc(Cl)cc(COc3ccccc3CC(=O)OC(C)(C)C)c2)c1F]

The title compound was synthesized in a similar manner as described in Intermediate 114-B starting with tert-butyl 2-(2-((3-bromo-5-chlorobenzyl)oxy)phenyl)acetate (Intermediate 53) and (R)-tert-butyl 2-(2-((3′-(1-((tert-butoxycarbonyl)amino)ethyl)-2′-fluoro-5-hydroxy-[1,1′-biphenyl]-3-yl)methoxy)phenyl)acetate (intermediate 27-B). MS (ESI+) m/z 592.4, 594.4 (M+Na).

### Intermediate 118

### (R)-tert-butyl 2-(2-((3′-(1-((tert-butoxycarbonyl)amino)ethyl)-2′-fluoro-5-((2-fluoroethyl)amino)-[1,1′-biphenyl]-3-yl)methoxy)phenyl)acetate

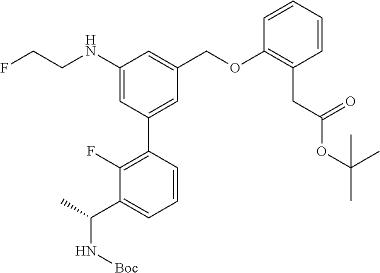 [SMILES: C[C@@H](NC(=O)OC(C)(C)C)c1cccc(-c2cc(COc3ccccc3CC(=O)OC(C)(C)C)cc(NCCF)c2)c1F]

A predegassed suspension of (R)-tert-butyl 2-(2-((3′-(1-((tert-butoxycarbonyl)amino)ethyl)-5-chloro-2′-fluoro-[1,1′-biphenyl]-3-yl)methoxy)phenyl)acetate (Intermediate 117) (100 mg, 0.175 mmol), 2-fluoroethanamine (12.17 mg, 0.193 mmol), BrettPhos palladacycle (CAS #1148148-01-9) (7.01 mg, 8.77 μmol) and Cs₂CO₃ (171 mg, 0.526 mmol) in MeCN (2 ml) in a sealed vial was heated in a microwave at 110° C. for 60 min. At this point, it was cooled to room temperature, filtered, and concentrated. The residue purified by flash column (0-50% EtOAc/Heptane) to give title compound. MS (ESI+) m/z 619.5 (M+Na).

### Intermediate 119

The intermediates below were synthesized in a similar manner as described in Intermediate 118 starting with (R)-tert-butyl 2-(2-((3′-(1-((tert-butoxycarbonyl)amino)ethyl)-5-chloro-2′-fluoro-[1,1′-biphenyl]-3-yl)methoxy)phenyl)acetate (Intermediate 117) and the appropriate amine.

| | | | MS (ESI+) |
|---|---|---|---|
| Intermediate | Structure/Chemical Name | amine | m/z |
| Intermediate 119-A | 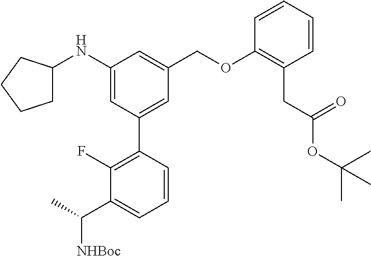 [SMILES: C[C@@H](NC(=O)OC(C)(C)C)c1cccc(-c2cc(COc3ccccc3CC(=O)OC(C)(C)C)cc(NC3CCCC3)c2)c1F] | 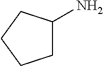 cyclopentanamine | 619.4 (M + H) |
| | (R)-tert-butyl 2-(2-((3′-(1-((tert- | | |
| | butoxycarbonyl)amino)ethyl)-5- | | |
| | (cyclopentylamino)-2′-fluoro-[1,1- | | |
| | biphenyl]-3- | | |
| | yl)methoxy)phenyl)acetate | | |
| Intermediate 119-B | 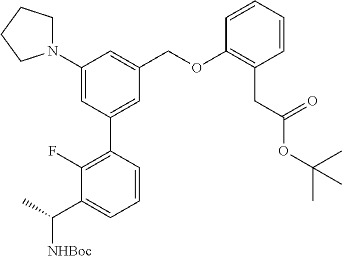 [SMILES: C[C@@H](NC(=O)OC(C)(C)C)c1cccc(-c2cc(COc3ccccc3CC(=O)OC(C)(C)C)cc(N3CCCC3)c2)c1F] | 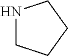 pyrrolidine | 605.4 (M + H) |
| | (R)-tert-butyl 2-(2-((3′-(1-((tert- | | |
| | butoxycarbonyl)amino)ethyl)-2′- | | |
| | fluoro-5-(pyrrolidin-1-yl)-[1,1′- | | |
| | biphenyl]-3- | | |
| | yl)methoxy)phenyl)acetate | | |
| Intermediate 119-C | 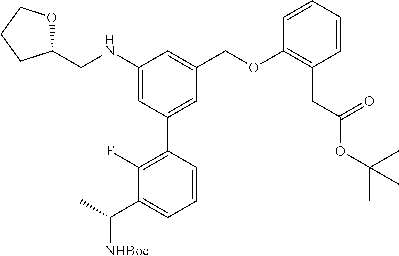 [SMILES: C[C@@H](NC(=O)OC(C)(C)C)c1cccc(-c2cc(COc3ccccc3CC(=O)OC(C)(C)C)cc(NC[C@@H]3CCCO3)c2)c1F] | 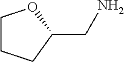 (S)- (tetrahydrofuran- 2-yl)methanamine | 635.3 (M + H) |
| | tert-butyl 2-(2-((3′-((R)-1-((tert- | | |
| | butoxycarbonyl)amino)ethyl)-2′- | | |
| | fluoro-5-((((S)-tetrahydrofuran-2- | | |
| | yl)methyl)amino)-[1,1′-biphenyl]-3- | | |
| | yl)methoxy)phenyl)acetate | | |
| Intermediate 119-D | 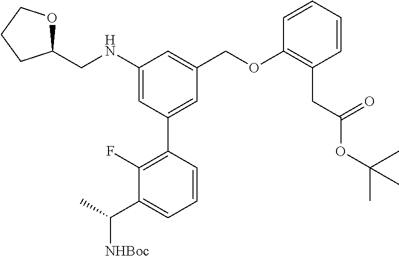 [SMILES: C[C@@H](NC(=O)OC(C)(C)C)c1cccc(-c2cc(COc3ccccc3CC(=O)OC(C)(C)C)cc(NC[C@H]3CCCO3)c2)c1F] | 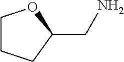 (R)- (tetrahydrofuran- 2-yl)methanamine | 635.3 (M + H) |
| | tert-butyl 2-(2-((3′-((R)-1-((tert- | | |
| | butoxycarbonyl)amino)ethyl)-2′- | | |
| | fluoro-5-((((R)-tetrahydrofuran-2- | | |
| | yl)methyl)amino)-[1,1′-biphenyl]-3- | | |
| | yl)methoxy)phenyl)acetate | | |

### Intermediate 120

### Tert-Butyl 2-(2-((3-chloro-5-((cyclopropylmethyl)amino)benzyl)oxy)phenyl)acetate

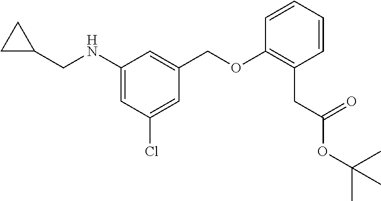 [SMILES: CC(C)(C)OC(=O)Cc1ccccc1OCc1cc(Cl)cc(NCC2CC2)c1]

A predegassed suspension of tert-Butyl 2-(2-((3-bromo-5-chlorobenzyl)oxy)phenyl)acetate (Intermediate 53) (440 mg, 1.069 mmol), cyclopropylmethanamine (99 mg, 1.389 mmol), BrettPhos palladacycle (CAS #1148148-01-9) (42.7 mg, 0.053 mmol) and Cs₂CO₃ (1045 mg, 3.21 mmol) in MeCN (10 ml) in a sealed vial was heated in a microwave at 110° C. for 60 min. It was then cooled to room temperature, filtered, and concentrated. The residue purified by flash column (0-50% EtOAc/Heptane) to give the title product. MS (ESI+) m/z 346.1, 348.1 (M-t-butyl).

### Intermediate 121

The following compounds were prepared with a similar method as described in Intermediate 120 using tert-butyl 2-(2-((3-bromo-5-chlorobenzyl)oxy)phenyl)acetate (Intermediate 53) and the appropriate amine as starting materials.

| | | | MS (ESI+) |
|---|---|---|---|
| | Structure/Chemical Name | amine | m/z |
| 121-A | 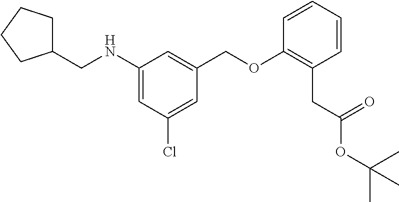 [SMILES: CC(C)(C)OC(=O)Cc1ccccc1OCc1cc(Cl)cc(NCC2CCCC2)c1] | 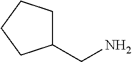 cyclopentylmethanamine | 430.4, 432.4 (M + H) |
| | tert-butyl 2-(2-((3-chloro-5- | | |
| | ((cyclopentylmethyl)amino) | | |
| | benzyl)oxy)phenyl)acetate | | |
| 121-B | 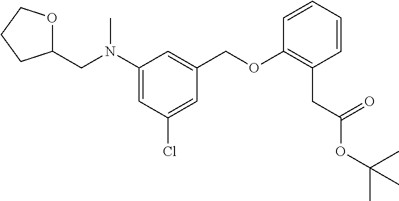 [SMILES: CN(CC1CCCO1)c1cc(Cl)cc(COc2ccccc2CC(=O)OC(C)(C)C)c1] | 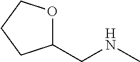 N-methyl-1- (tetrahydrofuran-2- yl)methanamine | 446.3, 448.3 (M + H) |
| | tert-butyl 2-(2-((3-chloro-5- | | |
| | (methyl((tetrahydrofuran-2- | | |
| | yl)methyl)amino)benzyl)oxy)phenyl) | | |
| | acetate | | |
| 121-C | 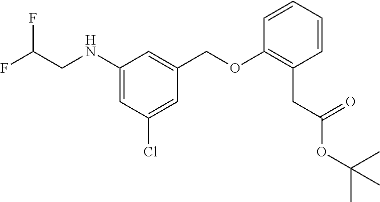 [SMILES: CC(C)(C)OC(=O)Cc1ccccc1OCc1cc(Cl)cc(NCC(F)F)c1] | 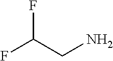 2,2-difluoroethanamine | 356.2 358.2 (M-t-butyl) |
| | tert-butyl 2-(2-((3-chloro-5-((2,2- | | |
| | difluoroethyl)amino)benzyl)oxy)phenyl)acetate | | |
| 121-D | 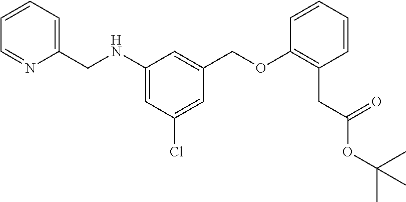 [SMILES: CC(C)(C)OC(=O)Cc1ccccc1OCc1cc(Cl)cc(NCc2ccccn2)c1] | 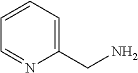 pyridin-2-ylmethanamine | 439.2, 441.2 (M +H ) |
| | tert-butyl 2-(2-((3-chloro-5-((pyridin- | | |
| | 2- | | |
| | ylmethyl)amino)benzyl)oxy)phenyl) | | |
| | acetate | | |

### Intermediate 122

### tert-Butyl 2-(2-((3-chloro-5-(2-(pyridin-4-yl)vinyl)benzyl)oxy)phenyl)acetate

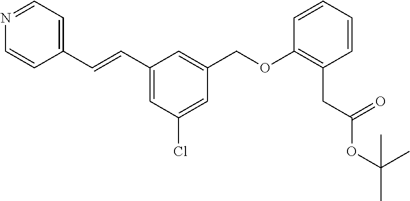 [SMILES: CC(C)(C)OC(=O)Cc1ccccc1OCc1cc(Cl)cc(/C=C/c2ccncc2)c1]

In a 2-5 mL microwave vial was placed tert-butyl 2-(2-((3-bromo-5-chlorobenzyl)oxy)phenyl)acetate (Intermediate 53) (1000 mg, 2.429 mmol) and 4-vinylpyridine (511 mg, 4.86 mmol) in DMF (10 ml). Then tri-o-tolylphosphine (111 mg, 0.364 mmol), triethylamine (1.016 ml, 7.29 mmol) and palladium (II) acetate (82 mg, 0.364 mmol) were added and the vial was sealed and heated in a microwave at 140° C. for 2 hours. The mixture was diluted with 100 mL 70% EtOAc/Heptane. Washed with 100 mL water. The organic layer was concentrated and the residue was purified with flash column (0-100% EtOAc/Heptane) to provide the title compound. MS (ESI+) m/z 436.3, 438.3 (M+H).

### Intermediate 123

### Tert-Butyl 2-(2-((3-((cyclopropylmethyl)amino)-5-(4,4,5,5-tetramethyl-1,3,2-dioxaborolan-2-yl)benzyl)oxy)phenyl)acetate

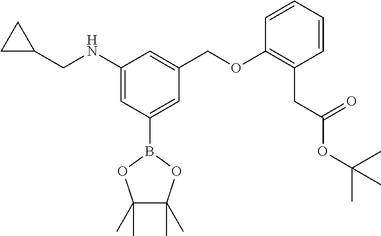 [SMILES: CC(C)(C)OC(=O)Cc1ccccc1OCc1cc(NCC2CC2)cc(B2OC(C)(C)C(C)(C)O2)c1]

To a solution of tert-butyl 2-(2-((3-chloro-5-((cyclopropylmethyl)amino)benzyl)oxy)phenyl)acetate (Intermediate 120) (200 mg, 0.498 mmol) in N,N-Dimethylacetamide (5 mL), 4,4,4′,4′,5,5,5′,5′-octamethyl-2,2′-bi(1,3,2-dioxaborolane) (190 mg, 0.746 mmol), potassium acetate (147 mg, 1.493 mmol), SPhos palladacycle (Cas #1028206-58-7) (16.73 mg, 0.025 mmol) were added and the reaction heated at 120° C. for 1 hour. Cooled to room temperature, poured into water, extracted with EtOAc/Heptane (50%) twice, evaporated and purified using FCC (0-30% EtOAc/Heptane) to provide title product. MS (ESI+) m/z 494.4 (M+H).

### Intermediate 124

The following compounds were prepared with similar methods as described in Intermediate 123 using the appropriate phenyl chloride.

| | Structure/Chemical Name | MS (ESI+) m/z |
|---|---|---|
| 124-A | 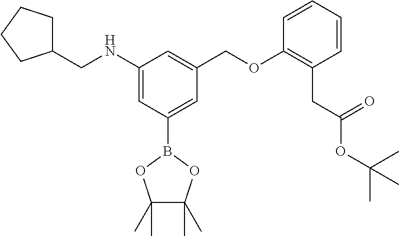 [SMILES: CC(C)(C)OC(=O)Cc1ccccc1OCc1cc(NCC2CCCC2)cc(B2OC(C)(C)C(C)(C)O2)c1] | 522.6 (M + H) |
| | tert-butyl 2-(2-((3- | |
| | ((cyclopentylmethyl)amino)-5- | |
| | (4,4,5,5-tetramethyl-1,3,2- | |
| | dioxaborolan-2- | |
| | yl)benzyl)oxy)phenyl)acetate | |
| 124-B | 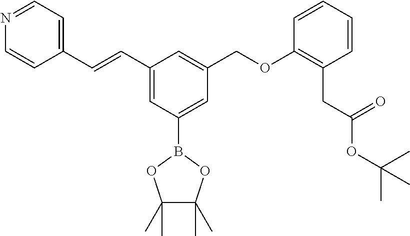 [SMILES: CC(C)(C)OC(=O)Cc1ccccc1OCc1cc(/C=C/c2ccncc2)cc(B2OC(C)(C)C(C)(C)O2)c1] | 528.5 (M + H) |
| | tert-butyl 2-(2-((3-(2-(pyridin-4- | |
| | yl)vinyl)-5-(4,4,5,5-tetramethyl-1,3,2- | |
| | dioxaborolan-2- | |
| | yl)benzyl)oxy)phenyl)acetate | |

### Intermediate 125

### tert-Butyl 2-(2-((3-chloro-5-((cyclopentylmethyl)(methyl)amino)benzyl)oxy)phenyl)acetate

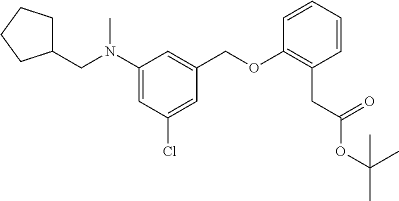 [SMILES: CN(CC1CCCC1)c1cc(Cl)cc(COc2ccccc2CC(=O)OC(C)(C)C)c1]

To a solution of tert-butyl 2-(2-((3-chloro-5-((cyclopentylmethyl)amino)benzyl)oxy)phenyl)acetate (Intermediate 121-A) (0.107 g, 0.25 mmol) in MeOH (3 mL) was added formaldehyde (0.101 mL, 1.250 mmol). Several drops 3M HCl were added to adjust pH around 6. Polymer bound zinc borohydride (0.119 g, 1.250 mmol) was added and the reaction mixture was stirred at room temperature for 72 hours, it was then filtered and concentrated and it was used on next step without purification. MS (ESI+) m/z 444.3, 446.3 (M+H).

### Intermediate 126

### Intermediate 126-A

### 8-Bromo-6-chlorochroman-4-ol

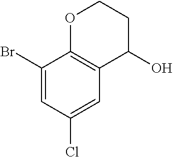 [SMILES: OC1CCOc2c(Br)cc(Cl)cc21]

NaBH₄ (0.145 g, 3.82 mmol) was added to a solution of 8-bromo-6-chlorochroman-4-one (1 g, 3.82 mmol) in MeOH (15 mL) and the mixture was stirred at rt overnight. LC-MS showed reaction completed. The mixture was concentrated and the residue was partitioned between EtOAc and sat. NH₄Cl. The aqueous layer was extracted with EtOAc. The combined organics were washed with brine, dried (Na₂SO₄) and concentrated. The residue was purified by ISCO (EtOAc-heptane 0-100%) to provide the desired product as an off-white solid. ¹H NMR (400 MHz, METHANOL-d₄) δ ppm 7.43 (d, J=2.5 Hz, 1H), 7.32 (d, J=2.5 Hz, 1H), 4.71 (t, J=4.8 Hz, 1H), 4.42-4.25 (m, 2H), 2.10 (m, 1H), 2.03-1.92 (m, 1H).

### Intermediate 126-B

### tert-Butyl 2-(2-((8-bromo-6-chlorochroman-4-yl)oxy)phenyl)acetate

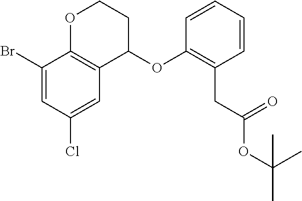 [SMILES: CC(C)(C)OC(=O)Cc1ccccc1OC1CCOc2c(Br)cc(Cl)cc21]

DIAD (0.266 mL, 1.366 mmol) was added dropwise to a solution of 8-bromo-6-chlorochroman-4-ol (300 mg, 1.138 mmol), tert-butyl 2-(2-hydroxyphenyl)acetate (Intermediate 21) (285 mg, 1.366 mmol) and PPh₃ (358 mg, 1.366 mmol) in THF (7.5 mL) at 0° C. The resulting yellow solution was allowed to warm to room temperature and then stirred overnight. Sat. NH₄Cl was added and the mixture was extracted with EtOAc (2×). The combined organics were washed with brine, dried (Na₂SO₄) and concentrated. The residue was purified by ISCO (EtOAc-heptane 0-50%) to provide the desired product. MS (ESI+) m/z 453.0, 455.0 (M+H).

### Intermediate 126-C

### tert-Butyl 2-(2-((6-chloro-8-((((R)-tetrahydrofuran-2-yl)methyl)amino)chroman-4-yl)oxy)phenyl)acetate (mixture of diastereomers)

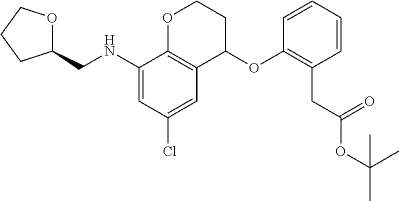 [SMILES: CC(C)(C)OC(=O)Cc1ccccc1OC1CCOc2c(NC[C@H]3CCCO3)cc(Cl)cc21]

A white suspension of tert-butyl 2-(2-((8-bromo-6-chlorochroman-4-yl)oxy)phenyl)acetate (190 mg, 0.293 mmol), (R)-(tetrahydrofuran-2-yl)methanamine (44.5 mg, 0.440 mmol), BrettPhos palladacycle (CAS #1148148-01-9) (11.71 mg, 0.015 mmol), Cs₂CO₃ (287 mg, 0.879 mmol) in acetonitrile (3 mL) was heated at 110° C. for 60 min in a microwave. The green reaction mixture was allowed to cool to room temperature and partitioned between EtOAc and water. The aqueous layer was extracted with EtOAc. The combined organics were washed, dried, and concentrated. The residue was purified by silica gel chromatography (0-40% EtOAc/Heptane) to provide the desired product.

### Intermediate 127

### tert-Butyl 2-(2-((6-chloro-8-((cyclopropylmethyl)amino)chroman-4-yl)oxy)phenyl)acetate (mixture of diastereomers)

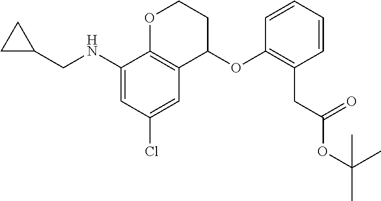 [SMILES: CC(C)(C)OC(=O)Cc1ccccc1OC1CCOc2c(NCC3CC3)cc(Cl)cc21]

The title compound was synthesized from cyclopropylmethanamine following similar procedure as shown in Intermediate 126-C.

### Intermediate 128

### tert-Butyl 2-(2-((7-chloro-5-((((R)-tetrahydrofuran-2-yl)methyl)amino)-1,2,3,4-tetrahydronaphthalen-1-yl)oxy)phenyl)acetate (mixture of diastereomers)

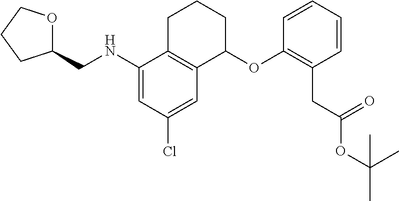 [SMILES: CC(C)(C)OC(=O)Cc1ccccc1OC1CCCc2c(NC[C@H]3CCCO3)cc(Cl)cc21]

The title compound was synthesized from 5-bromo-7-chloro-3,4-dihydronaphthalen-1(2H)-one following similar procedure as shown in Intermediate 126-C.

### Intermediate 129

### tert-Butyl 2-(2-((7-chloro-5-((cyclopropylmethyl)amino)-1,2,3,4-tetrahydronaphthalen-1-yl)oxy)phenyl)acetate (mixture of diastereomers)

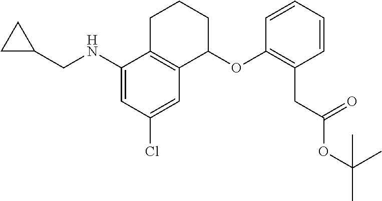 [SMILES: CC(C)(C)OC(=O)Cc1ccccc1OC1CCCc2c(NCC3CC3)cc(Cl)cc21]

The title compound was synthesized from cyclopropylmethanamine following similar procedure as shown in Intermediate 126-C.

### Intermediate 130

### Intermediate 130-A

### Methyl 3-bromo-5-(dimethylcarbamoyl)benzoate

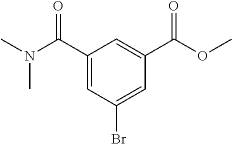

NaOH (1N aqueous solution, 4.87 mL, 4.87 mmol) was added to a solution of dimethyl 5-bromoisophthalate (1.33 g, 4.87 mmol) in MeOH (25 mL)/THF (25 mL). The reaction mixture was stirred at room temperature overnight. Excess base was then quenched with 1N HCl. The resulting mixture was extracted with EtOAc. The organic layer was dried and concentrated. The crude acid was suspended in dichloromethane (50 mL), and oxalyl chloride (0.512 mL, 5.84 mmol) and DMF (0.038 mL, 0.487 mmol) were added at 0° C. After 15 minutes, dimethylamine in ethanol (8.70 mL, 48.7 mmol) was added. The reaction mixture was stirred for 2 h and concentrated. The residue was purified using silica gel chromatography (EtOAc-heptane 0-60%) to provide the desired product. MS (ESI+) m/z 386.0, 388.0 (M+1).

### Intermediate 130-B

### 3-Bromo-5-(hydroxymethyl)-N,N-dimethylbenzamide

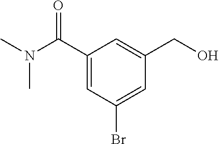

LiBH₄ (55.6 mg, 2.55 mmol) was added to a solution of methyl 3-bromo-5-(dimethylcarbamoyl)benzoate (730 mg, 2.55 mmol) in THF (15 mL) at rt, followed by MeOH (0.103 mL, 2.55 mmol). After 4 hr additional LiBH₄ (55.6 mg) was added, followed by MeOH (0.103 mL). After another 2 hr at rt, sat. NH₄Cl was added and resulting mixture was extracted with EtOAc (2×). The combined organics were washed with brine, dried (Na₂SO₄), and concentrated. The residue was purified by ISCO (EtOAc-heptane 0-100%) to provide the desired product as a white solid. MS (ESI+) m/z 258.0, 260.0 (M+1).

### Intermediate 130-C

### tert-Butyl 2-(2-((3-bromo-5-(dimethylcarbamoyl)benzyl)oxy)phenyl)acetate

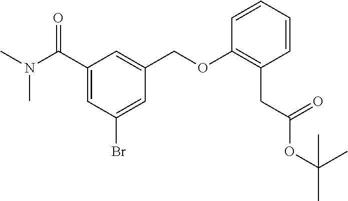 [SMILES: CN(C)C(=O)c1cc(Br)cc(COc2ccccc2CC(=O)OC(C)(C)C)c1]

The title compound was synthesized from 3-bromo-5-(hydroxymethyl)-N,N-dimethylbenzamide following similar procedure as shown in Intermediate 126-B.

### Intermediate 130-D

### (R)-tert-Butyl 2-(2-((3′-(1-((tert-butoxycarbonyl)amino)ethyl)-5-(dimethylcarbamoyl)-2′-fluoro-[1,1′-biphenyl]-3-yl)methoxy)phenyl)acetate

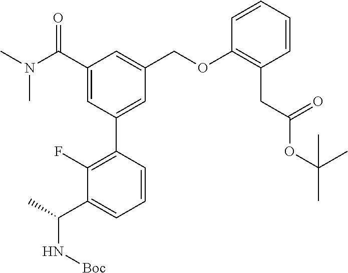 [SMILES: C[C@@H](NC(=O)OC(C)(C)C)c1cccc(-c2cc(COc3ccccc3CC(=O)OC(C)(C)C)cc(C(=O)N(C)C)c2)c1F]

In a 2-5 mL microwave vial was placed tert-butyl 2-(2-((3-bromo-5-(dimethylcarbamoyl)benzyl)oxy)phenyl)acetate (142.8 mg, 0.319 mmol), (R)-tert-butyl (1-(2-fluoro-3-(4,4,5,5-tetramethyl-1,3,2-dioxaborolan-2-yl)phenyl)ethyl)carbamate (Intermediate 27B) (128 mg, 0.350 mmol), PdCl₂(dppf).CH₂Cl₂ adduct (CAS #95464-05-4) (13.00 mg, 0.016 mmol) in Acetonitrile (2 mL). K₃PO₄ (2M solution) (0.478 ml, 0.956 mmol) was added. Nitrogen was blown above the solvent and the vial was sealed and microwaved at 100° C. for 60 min. The dark reaction mixture was cooled to rt, the organic layer was filtered and purified by flash chromatography (EtOAc-heptane 0-70%) to give the title compound. MS (ESI+) m/z 607.5 (M+H).

### Intermediate 131

### (+) and (−)-tert-Butyl 2-(2-((3′-(1-amino-2-fluoroethyl)-5-((cyclopropylmethyl)amino)-2′-fluoro-[1,1′-biphenyl]-3-yl)methoxy)phenyl)acetate

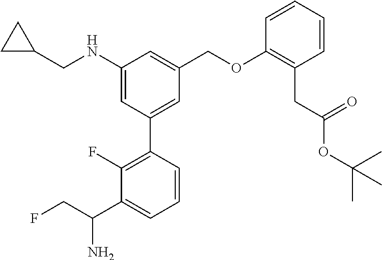 [SMILES: CC(C)(C)OC(=O)Cc1ccccc1OCc1cc(NCC2CC2)cc(-c2cccc(C(N)CF)c2F)c1]

In a 2-5 mL microwave vial was placed tert-butyl 2-(2-((3-((cyclopropylmethyl)amino)-5-(4,4,5,5-tetramethyl-1,3,2-dioxaborolan-2-yl)benzyl)oxy)phenyl)acetate (Intermediate 123) (0.19 g, 0.385 mmol), (±)-1-(3-Bromo-2-fluorophenyl)-2-fluoroethanamine (Intermediate 33-B). (0.136 g, 0.501 mmol), PdCl₂(dppf).CH₂Cl₂ adduct (CAS #95464-05-4) (0.014 g, 0.019 mmol) in acetonitrile (3 mL). K₃PO₄ (2M aq. solution, 0.963 ml, 1.925 mmol) was added and the vial was sealed and heated in a microwave at 110° C. for 60 min. The reaction mixture was cooled to rt. The organic layer was filtered and purified by flash chromatography (EtOAc-Heptane 0-100%) to give the title compound. MS (ESI+) m/z 523.4 (M+H). Resolution of (±)-tert-butyl 2-(2-((3′-(1-amino-2-fluoroethyl)-5-((cyclopropylmethyl)amino)-2′-fluoro-[1,1′-biphenyl]-3-yl)methoxy)phenyl)acetate was achieved by chiral SFC using a CHIRALPAK® OJ-H column with 30% MeOH and 5 mM NH₄OH in CO₂ to give the first enantiomer Intermediate 131-A1 (tr=4.1 min) and the second enantiomer Intermediate 131-A2 (tr=6.0 min).

### Intermediate 132

The following compounds were prepared with similar methods as described in Intermediate 131 by using appropriate borate and aryl bromide.

| | | | Aryl | MS (ESI+) |
|---|---|---|---|---|
| | Structure/Chemical Name | borate | bromide | m/z |
| 132-A | 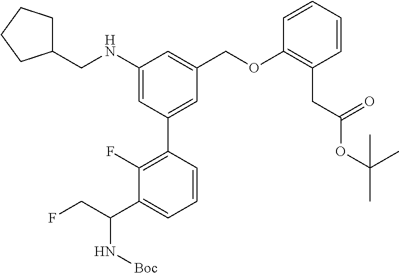 [SMILES: CC(C)(C)OC(=O)Cc1ccccc1OCc1cc(NCC2CCCC2)cc(-c2cccc(C(CF)NC(=O)OC(C)(C)C)c2F)c1] | Intermediate 124-A | Intermediate 33-C2 | 651.6 (M + H) |
| | tert-butyl 2-(2-((3′-(1-((tert- | | | |
| | butoxycarbonyl)amino)-2- | | | |
| | fluoroethyl)-5- | | | |
| | ((cyclopentylmethyl)amino)-2′- | | | |
| | fluoro-[1,1-biphenyl]-3- | | | |
| | yl)methoxy)phenyl)acetate | | | |
| 132-B | 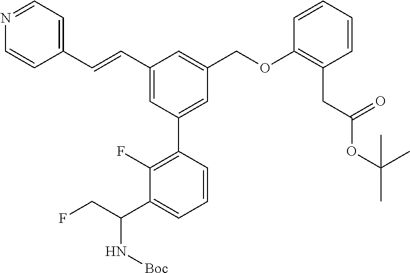 [SMILES: CC(C)(C)OC(=O)Cc1ccccc1OCc1cc(/C=C/c2ccncc2)cc(-c2cccc(C(CF)NC(=O)OC(C)(C)C)c2F)c1] | Intermediate 124-B | Intermediate 33-C2 | 657.6 (M + H) |
| | (±)-tert-butyl 2-(2-((3′-(1-((tert- | | | |
| | butoxycarbonyl)amino)-2- | | | |
| | fluoroethyl)-2′-fluoro-5-(2-(pyridin- | | | |
| | 4-yl)vinyl)-[1,1′-biphenyl]-3- | | | |
| | yl)methoxy)phenyl)acetate | | | |

### Intermediate 133

### (R)-tert-Butyl 2-(2-((3′-(1-((tert-butoxycarbonyl)amino)ethyl)-5-((cyclopentylmethyl)amino)-2′-fluoro-[1,1′-biphenyl]-3-yl)methoxy)phenyl)acetate

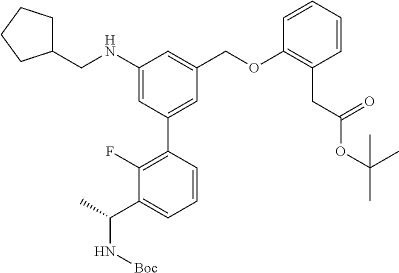 [SMILES: C[C@@H](NC(=O)OC(C)(C)C)c1cccc(-c2cc(COc3ccccc3CC(=O)OC(C)(C)C)cc(NCC3CCCC3)c2)c1F]

In a 2-5 mL microwave vial was placed tert-butyl 2-(2-((3-chloro-5-((cyclopentylmethyl)amino)benzyl)oxy)phenyl)acetate (Intermediate 124-A) (100 mg, 0.233 mmol) and (R)-tert-butyl (1-(2-fluoro-3-(4,4,5,5-tetramethyl-1,3,2-dioxaborolan-2-yl)phenyl)ethyl)carbamate (Intermediate 27-B) (110 mg, 0.302 mmol) in MeCN (2 ml). Then K₃PO₄ (2M solution) (0.349 ml, 0.698 mmol) and SPhos palladacycle (Cas #1028206-58-7) (7.82 mg, 0.012 mmol) was added and the vial was sealed and heated in a microwave at 110° C. for 60 min. The reaction mixture was cooled to rt. The organic layer was filtered and the filtrate was concentrated. The residue was purified by flash column 0-50% EtOAC/heptane to provide title compound. MS (ESI+) m/z 633.6 (M+H).

### Intermediate 134

The following compounds were prepared with similar methods as described in Intermediate 133 by using appropriate phenyl chloride and borate.

| | Structure/Chemical Name | MS (ESI+) m/z |
|---|---|---|
| 134-A | 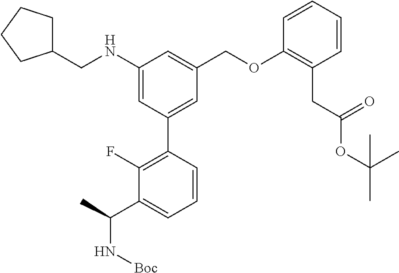 [SMILES: C[C@H](NC(=O)OC(C)(C)C)c1cccc(-c2cc(COc3ccccc3CC(=O)OC(C)(C)C)cc(NCC3CCCC3)c2)c1F] | 633.6 (M + H) |
| | (S)-tert-butyl 2-(2-((3′-(1-((tert-butoxycarbonyl)amino)ethyl)-5- | |
| | ((cyclopentylmethyl)amino)-2′-fluoro-[1,1-biphenyl]-3- | |
| | yl)methoxy)phenyl)acetate | |
| 134-B | 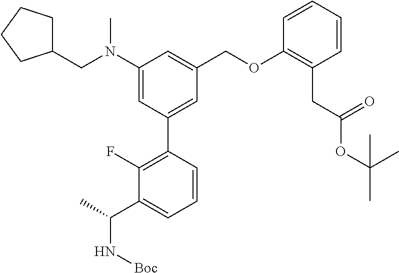 [SMILES: C[C@@H](NC(=O)OC(C)(C)C)c1cccc(-c2cc(COc3ccccc3CC(=O)OC(C)(C)C)cc(N(C)CC3CCCC3)c2)c1F] | 647.6 (M + H) |
| | (R)-tert-butyl 2-(2-((3′-(1-((tert-butoxycarbonyl)amino)ethyl)-5- | |
| | ((cyclopentylmethyl)(methyl)amino)-2′-fluoro-[1,1-biphenyl]-3- | |
| | yl)methoxy)phenyl)acetate | |
| 134-C | 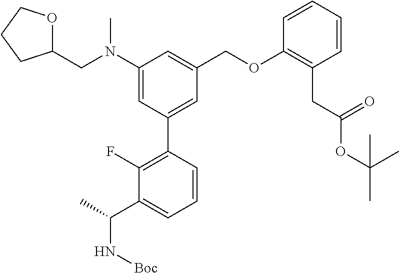 [SMILES: C[C@@H](NC(=O)OC(C)(C)C)c1cccc(-c2cc(COc3ccccc3CC(=O)OC(C)(C)C)cc(N(C)CC3CCCO3)c2)c1F] | 649.6 (M + H) |
| | tert-butyl 2-(2-((3′-((R)-1-((tert-butoxycarbonyl)amino)ethyl)-2′- | |
| | fluoro-5-(methyl((tetrahydrofuran-2-yl)methyl)amino)-[1,1′- | |
| | biphenyl]-3-yl)methoxy)phenyl)acetate | |
| 134-D | 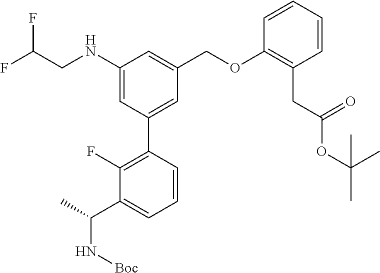 [SMILES: C[C@@H](NC(=O)OC(C)(C)C)c1cccc(-c2cc(COc3ccccc3CC(=O)OC(C)(C)C)cc(NCC(F)F)c2)c1F] | 637.5 (M + Na) |
| | (R)-tert-butyl 2-(2-((3′-(1-((tert-butoxycarbonyl)amino)ethyl)-5- | |
| | ((2,2-difluoroethyl)amino)-2′-fluoro-[1,1′-biphenyl]-3- | |
| | yl)methoxy)phenyl)acetate | |
| 134-E | 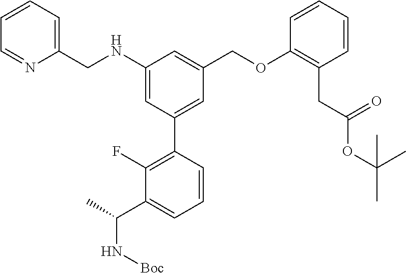 [SMILES: C[C@@H](NC(=O)OC(C)(C)C)c1cccc(-c2cc(COc3ccccc3CC(=O)OC(C)(C)C)cc(NCc3ccccn3)c2)c1F] | 642.6 (M + H) |
| | (R)-tert-butyl 2-(2-((3′-(1-((tert-butoxycarbonyl)amino)ethyl)-2′- | |
| | fluoro-5-((pyridin-2-ylmethyl)amino)-[1,1′-biphenyl]-3- | |
| | yl)methoxy)phenyl)acetate | |
| 134-F | 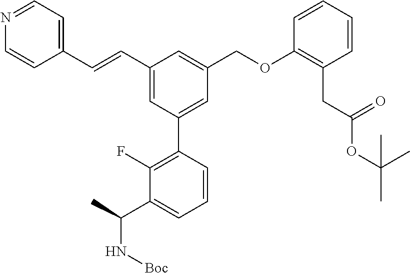 [SMILES: C[C@H](NC(=O)OC(C)(C)C)c1cccc(-c2cc(/C=C/c3ccncc3)cc(COc3ccccc3CC(=O)OC(C)(C)C)c2)c1F] | 639.5 (M + H) |
| | (S)-tert-butyl 2-(2-((3′-(1-((tert-butoxycarbonyl)amino)ethyl)-2′- | |
| | fluoro-5-(2-(pyridin-4-yl)vinyl)-[1,1′-biphenyl]-3- | |
| | yl)methoxy)phenyl)acetate | |
| 134-G | 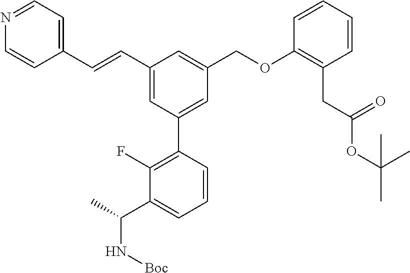 [SMILES: C[C@@H](NC(=O)OC(C)(C)C)c1cccc(-c2cc(/C=C/c3ccncc3)cc(COc3ccccc3CC(=O)OC(C)(C)C)c2)c1F] | 639.5 (M + H) |
| | (R)-tert-butyl 2-(2-((3′-(1-((tert-butoxycarbonyl)amino)ethyl)-2′- | |
| | fluoro-5-(2-(pyridin-4-yl)vinyl)-[1,1′-biphenyl]-3- | |
| | yl)methoxy)phenyl)acetate | |
| 134-H | 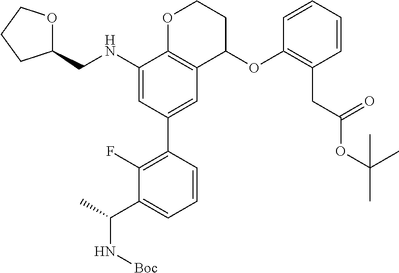 [SMILES: C[C@@H](NC(=O)OC(C)(C)C)c1cccc(-c2cc(NC[C@H]3CCCO3)c3c(c2)C(Oc2ccccc2CC(=O)OC(C)(C)C)CCO3)c1F] | 677.7 (M + H) |
| | tert-butyl 2-(2-((6-(3-((R)-1-((tert-butoxycarbonyl)amino)ethyl)- | |
| | 2-fluorophenyl)-8-((((R)-tetrahydrofuran-2- | |
| | yl)methyl)amino)chroman-4-yl)oxy)phenyl)acetate | |
| 134-I | 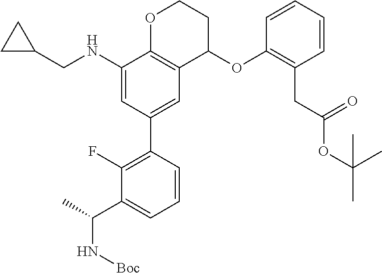 [SMILES: C[C@@H](NC(=O)OC(C)(C)C)c1cccc(-c2cc(NCC3CC3)c3c(c2)C(Oc2ccccc2CC(=O)OC(C)(C)C)CCO3)c1F] | 647.7 (M + H) |
| | tert-butyl 2-(2-((6-(3-((R)-1-((tert-butoxycarbonyl)amino)ethyl)- | |
| | 2-fluorophenyl)-8-((cyclopropylmethyl)amino)chroman-4- | |
| | yl)oxy)phenyl)acetate | |
| 134-J | 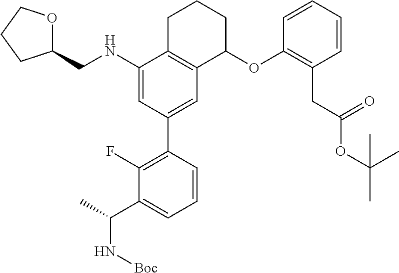 [SMILES: C[C@@H](NC(=O)OC(C)(C)C)c1cccc(-c2cc(NC[C@H]3CCCO3)c3c(c2)C(Oc2ccccc2CC(=O)OC(C)(C)C)CCC3)c1F] | 697.7 (M + Na) |
| | tert-butyl 2-(2-((7-(3-((R)-1-((tert-butoxycarbonyl)amino)ethyl)- | |
| | 2-fluorophenyl)-5-((((R)-tetrahydrofuran-2-yl)methyl)amino)- | |
| | 1,2,3,4-tetrahydronaphthalen-1-yl)oxy)phenyl)acetate | |
| 134-K | 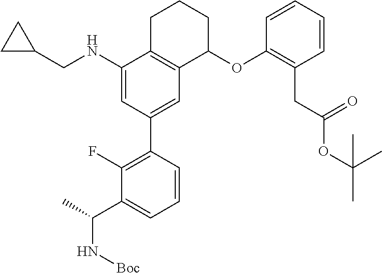 [SMILES: C[C@@H](NC(=O)OC(C)(C)C)c1cccc(-c2cc(NCC3CC3)c3c(c2)C(Oc2ccccc2CC(=O)OC(C)(C)C)CCC3)c1F] | 667.7 (M + Na) |
| | tert-butyl 2-(2-((7-(3-((R)-1-((tert-butoxycarbonyl)amino)ethyl)- | |
| | 2-fluorophenyl)-5-((cyclopropylmethyl)amino)-1,2,3,4- | |
| | tetrahydronaphthalen-1-yl)oxy)phenyl)acetate | |

### Intermediate 135

### (R)-tert-Butyl 2-(2-((3′-(1-((tert-butoxycarbonyl)amino)ethyl)-2′-fluoro-5-((2-fluoroethyl)(methyl)amino)-[1,1-biphenyl]-3-yl)methoxy)phenyl)acetate

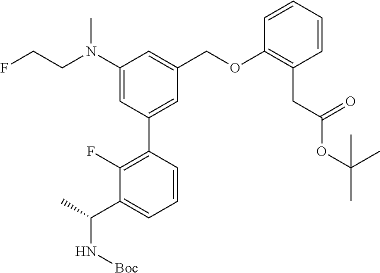 [SMILES: C[C@@H](NC(=O)OC(C)(C)C)c1cccc(-c2cc(COc3ccccc3CC(=O)OC(C)(C)C)cc(N(C)CCF)c2)c1F]

To a solution of (R)-tert-butyl 2-(2-((3′-(1-((tert-butoxycarbonyl)amino)ethyl)-2′-fluoro-5-((2-fluoroethyl)amino)-[1,1′-biphenyl]-3-yl)methoxy)phenyl)acetate (Intermediate 118) (0.091 g, 0.153 mmol) in CF₃CH₂OH (5 ml) was added paraformaldehyde (0.046 g, 1.525 mmol) and NaBH₄ (0.023 g, 0.610 mmol). After the mixture was heated to 90° C. for 30 min, another portion of paraformaldehyde (0.046 g, 1.525 mmol) and NaBH₄ (0.023 g, 0.610 mmol) was added. The mixture was heated at 90° C. for another 30 min. Cooled to room temperature, filtered and washed with CF₃CH₂OH, the filtrated was concentrated and the residue was purified by Flash column (0-30% EtOAc/Heptane) to provide title product. MS (ESI+) m/z 633.6 (M+Na).

### Intermediate 136

The following compounds were prepared with similar methods as described in 135 by using appropriate aniline and aldehyde.

| Intermediate | Structure/Chemical Name | MS (ESI+) m/z |
|---|---|---|
| 136-A | 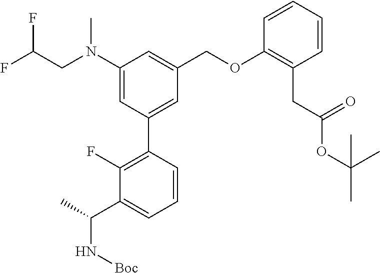 [SMILES: C[C@@H](NC(=O)OC(C)(C)C)c1cccc(-c2cc(COc3ccccc3CC(=O)OC(C)(C)C)cc(N(C)CC(F)F)c2)c1F] | 651.5 (M + Na). |
| | (R)-tert-butyl 2-(2-((3′-(1-((tert- | |
| | butoxycarbonyl)amino)ethyl)-5-((2,2- | |
| | difluoroethyl)(methyl)amino)-2′-fluoro-[1,1′-biphenyl]- | |
| | 3-yl)methoxy)phenyl)acetate | |
| 136-B | 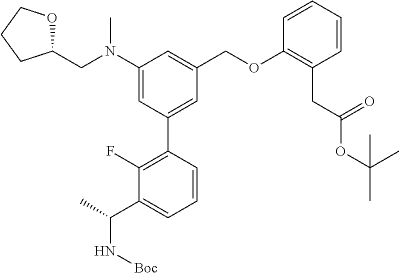 [SMILES: C[C@@H](NC(=O)OC(C)(C)C)c1cccc(-c2cc(COc3ccccc3CC(=O)OC(C)(C)C)cc(N(C)C[C@@H]3CCCO3)c2)c1F] | 649.5 (M + H). |
| | tert-butyl 2-(2-((3′-((R)-1-((tert- | |
| | butoxycarbonyl)amino)ethyl)-2′-fluoro-5-(methyl(((S)- | |
| | tetrahydrofuran-2-yl)methyl)amino)-[1,1′-biphenyl]-3- | |
| | yl)methoxy)phenyl)acetate | |
| 136-C | 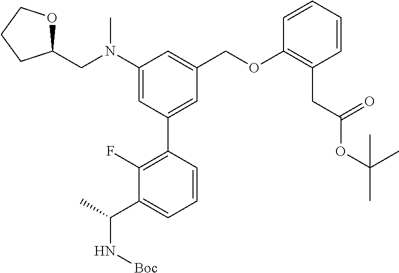 [SMILES: C[C@@H](NC(=O)OC(C)(C)C)c1cccc(-c2cc(COc3ccccc3CC(=O)OC(C)(C)C)cc(N(C)C[C@H]3CCCO3)c2)c1F] | 649.5 (M + H). |
| | tert-butyl 2-(2-((3′-((R)-1-((tert- | |
| | butoxycarbonyl)amino)ethyl)-2′-fluoro-5-(methyl(((R)- | |
| | tetrahydrofuran-2-yl)methyl)amino)-[1,1′-biphenyl]-3- | |
| | yl)methoxy)phenyl)acetate | |
| 136-D | 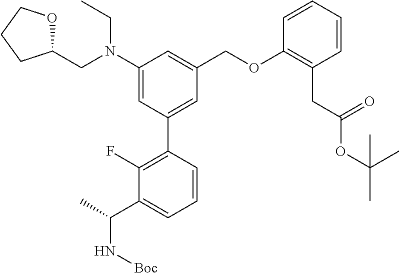 [SMILES: CCN(C[C@@H]1CCCO1)c1cc(COc2ccccc2CC(=O)OC(C)(C)C)cc(-c2cccc([C@@H](C)NC(=O)OC(C)(C)C)c2F)c1] | 663.5 (M + H). |
| | tert-butyl 2-(2-((3′-((R)-1-((tert- | |
| | butoxycarbonyl)amino)ethyl)-5-(ethyl(((S)- | |
| | tetrahydrofuran-2-yl)methyl)amino)-2′-fluoro-[1,1′- | |
| | biphenyl]-3-yl)methoxy)phenyl)acetate | |

### Intermediate 137

### (S)-tert-Butyl 2-(2-((3′-(1-((tert-butoxycarbonyl)amino)ethyl)-2′-fluoro-5-(2-(pyridin-4-yl)ethyl)-[1,1′-biphenyl]-3-yl)methoxy)phenyl)acetate

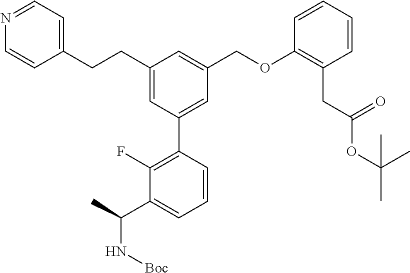 [SMILES: C[C@H](NC(=O)OC(C)(C)C)c1cccc(-c2cc(CCc3ccncc3)cc(COc3ccccc3CC(=O)OC(C)(C)C)c2)c1F]

To a solution of (S)-tert-butyl 2-(2-((3′-(1-((tert-butoxycarbonyl)amino)ethyl)-2′-fluoro-5-(2-(pyridin-4-yl)vinyl)-[1,1′-biphenyl]-3-yl)methoxy)phenyl)acetate (Intermediate 134-F) (0.2 g, 0.313 mmol) in EtOAc (5 mL) was added Pd-C (0.10 g, 0.094 mmol) and the mixture was degassed and placed under hydrogen for 2 hours. Several drops of NH₄OH were added. The mixture was filtered through a pad of celite, and washed with EtOAc. The filtrate was concentrated to provide title compound. MS (ESI+) m/z 641.6 (M+H).

### Intermediate 138

### (R)-tert-butyl 2-(2-((3′-(1-((tert-butoxycarbonyl)amino)ethyl)-2′-fluoro-5-(2-(pyridin-4-yl)ethyl)-[1,1′-biphenyl]-3-yl)methoxy)phenyl)acetate

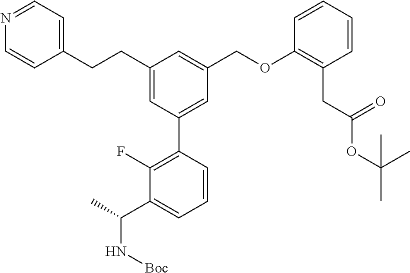 [SMILES: C[C@@H](NC(=O)OC(C)(C)C)c1cccc(-c2cc(CCc3ccncc3)cc(COc3ccccc3CC(=O)OC(C)(C)C)c2)c1F]

The title compound was synthesized in a similar manner as described in Intermediate 137 starting with (S)-tert-butyl 2-(2-((3′-(1-((tert-butoxycarbonyl)amino)ethyl)-5-((2,2-difluoroethyl)amino)-2′-fluoro-[1,1′-biphenyl]-3-yl)methoxy)phenyl)acetate (Intermediate 134-G). MS (ESI+) m/z 641.6 (M+H).

### Intermediate 139

### Tert-butyl 2-(2-((3′-(1-((tert-butoxycarbonyl)amino)-2-fluoroethyl)-2′-fluoro-5-(2-(pyridin-4-yl)ethyl)-[1,1′-biphenyl]-3-yl)methoxy)phenyl)acetate

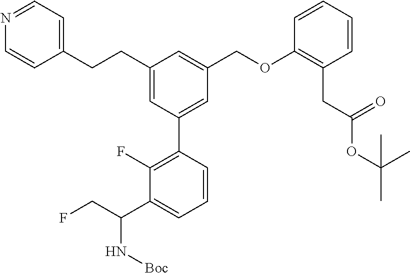 [SMILES: CC(C)(C)OC(=O)Cc1ccccc1OCc1cc(CCc2ccncc2)cc(-c2cccc(C(CF)NC(=O)OC(C)(C)C)c2F)c1]

The title compound was synthesized in a similar manner as described in Intermediate 137 starting with (tert-butyl 2-(2-((3′-(1-((tert-butoxycarbonyl)amino)-2-fluoroethyl)-2′-fluoro-5-(2-(pyridin-4-yl)vinyl)-[1,1′-biphenyl]-3-yl)methoxy)phenyl)acetate (Intermediate 132-B). MS (ESI+) m/z 659.6 (M+H).

### Intermediate 140

### Intermediate 140-A

### Methyl 2-(2-(((3-bromo-5-chlorophenyl)amino)methyl)phenyl)acetate

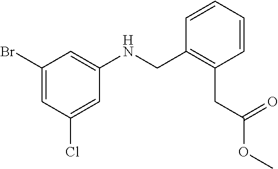 [SMILES: COC(=O)Cc1ccccc1CNc1cc(Cl)cc(Br)c1]

A solution of methyl 2-(2-formylphenyl)acetate (1 g, 5.61 mmol) in CF₃CH₂OH (10 ml) was stirred at 40° C. for 5 min. 3-bromo-5-chloroaniline (1.275 g, 6.17 mmol) was added and stirred vigorously. After 5 min, NaBH₄(0.276 g, 7.30 mmol) was added and the reaction was followed by LCMS. After 3 hours the reaction was filtered and washed by DCM; the filtrate was concentrated and the residue was purified by flash column (0-60% EtOAc/Heptane) to provide title compound. MS (ESI+) m/z 368.0, 370.0, 372.0 (M+H).

### Intermediate 140-B

### Methyl 2-(2-(((3-bromo-5-chlorophenyl)(methyl)amino)methyl)phenyl)acetate

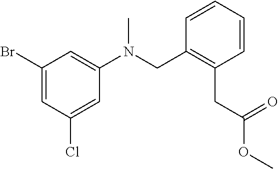 [SMILES: COC(=O)Cc1ccccc1CN(C)c1cc(Cl)cc(Br)c1]

To a solution of methyl 2-(2-(((3-bromo-5-chlorophenyl)amino)methyl)phenyl)acetate (1800 mg, 4.88 mmol) in CF₃CH₂OH (20 ml) was added paraformaldehyde (1466 mg, 48.8 mmol) and NaBH₄ (739 mg, 19.5 mmol). The mixture was heated to 90° C. for 30 min and at this point paraformaldehyde (460 mg, 15.3 mmol) and NaBH₄ (230 mg, 6.1 mmol) were added again. After 30 min the mixture was filtered and washed with CF₃CH₂OH and DCM. The filtrate was concentrated and the residue was purified by flash column (0-30% EtOAc/Heptane) to provide the title compound. MS (ESI+) m/z 382.0, 384.0, 386.0 (M+H).

### Intermediate 141

The following intermediates were prepared with similar methods as described in Intermediate 120 using methyl 2-(2-(((3-bromo-5-chlorophenyl)(methyl)amino)methyl)phenyl)acetate (Intermediate 140-B) and the appropriate amine as starting materials.

| | | | MS (ESI+) |
|---|---|---|---|
| Intermediate | Structure/Chemical Name | amine | m/z |
| 141-A | 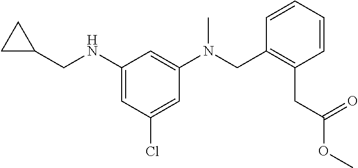 [SMILES: COC(=O)Cc1ccccc1CN(C)c1cc(Cl)cc(NCC2CC2)c1] | 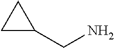 cyclopropylmethanamine | 373.0, 375.0 (M + H) |
| | methyl 2-(2-(((3-chloro-5- | | |
| | ((cyclopropylmethyl)amino) | | |
| | phenyl)(methyl)amino)methyl) | | |
| | phenyl)acetate | | |
| 141-B | 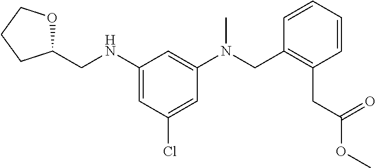 [SMILES: COC(=O)Cc1ccccc1CN(C)c1cc(Cl)cc(NC[C@@H]2CCCO2)c1] | 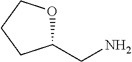 (S)-(tetrahydrofuran-2- yl)methanamine | 403.2, 405.2 (M + H) |
| | (S)-methyl 2-(2-(((3-chloro-5- | | |
| | (((tetrahydrofuran-2- | | |
| | yl)methyl)amino)phenyl)(methyl) | | |
| | amino)methyl)phenyl)acetate | | |
| 141-C | 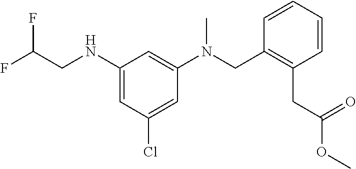 [SMILES: COC(=O)Cc1ccccc1CN(C)c1cc(Cl)cc(NCC(F)F)c1] | 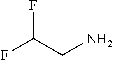 2,2-difluoroethanamine | 383.1, 385.1 (M + H) |
| | methyl 2-(2-(((3-chloro-5-((2,2- | | |
| | difluoroethyl)amino)phenyl) | | |
| | (methyl)amino)methyl)phenyl)acetate | | |

### Intermediate 142

### (R)-2-(2-(((3′-(1-((tert-butoxycarbonyl)amino)ethyl)-5-((cyclopropylmethyl)amino)-2′-fluoro-[1,1′-biphenyl]-3-yl)(methyl)amino)methyl)phenyl)acetic acid

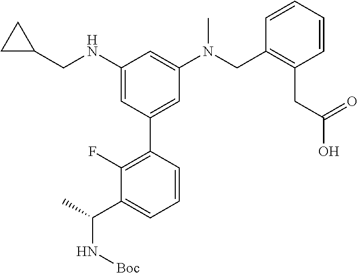 [SMILES: C[C@@H](NC(=O)OC(C)(C)C)c1cccc(-c2cc(NCC3CC3)cc(N(C)Cc3ccccc3CC(=O)O)c2)c1F]

In a 2-5 mL microwave vial was placed methyl 2-(2-(((3-chloro-5-((cyclopropylmethyl)amino)phenyl)(methyl)amino)methyl)phenyl)acetate (Intermediate 141-A) (200 mg, 0.536 mmol) and (R)-tert-butyl (1-(2-fluoro-3-(4,4,5,5-tetramethyl-1,3,2-dioxaborolan-2-yl)phenyl)ethyl)carbamate (Intermediate 27-B) (235 mg, 0.644 mmol) in MeCN (3 ml). Then K₃PO₄ (2M solution) (0.805 ml, 1.609 mmol) and SPhos palladacycle (Cas #1028206-58-7)(18.04 mg, 0.027 mmol) were added and the vial was sealed and heated in a microwave at 110° C. for 60 min. The reaction mixture was cooled to rt. The organic layer was filtered. 1M LiOH (1 mL) was added to the filtrate. The mixture was heated at 50° C. for 2 hours. 1M HCl (1 mL) was added and filtered. The filtrate was purified by HPLC (10-100% ACN/water with 0.1% NH₄OH) to provide title compound. MS (ESI+) m/z 562.3 (M+H).

### Intermediate 143

The following intermediates were prepared with similar methods as described in Intermediate 142 using appropriate phenyl chloride as starting materials.

| | | MS (ESI+) |
|---|---|---|
| Intermediate | Structure/Chemical Name | m/z |
| 143-A | 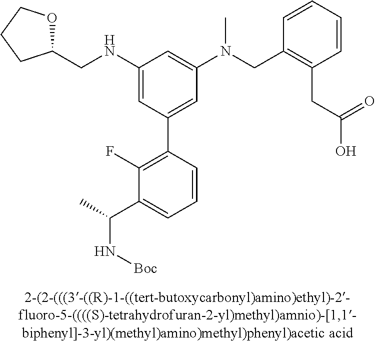 [SMILES: C[C@@H](NC(=O)OC(C)(C)C)c1cccc(-c2cc(NC[C@@H]3CCCO3)cc(N(C)Cc3ccccc3CC(=O)O)c2)c1F] | 592.3 (M + H) |
| 143-B | 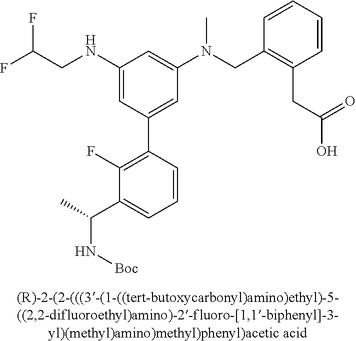 [SMILES: C[C@@H](NC(=O)OC(C)(C)C)c1cccc(-c2cc(NCC(F)F)cc(N(C)Cc3ccccc3CC(=O)O)c2)c1F] | 572.2 (M + H) |

### Intermediate 144

### (R)-tert-Butyl 2-(2-((3′-(1-((tert-butoxycarbonyl)amino)ethyl)-2′-fluoro-5-formyl-[1,1′-biphenyl]-3-yl)methoxy)phenyl)acetate

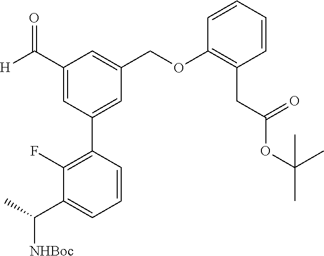 [SMILES: C[C@@H](NC(=O)OC(C)(C)C)c1cccc(-c2cc(C=O)cc(COc3ccccc3CC(=O)OC(C)(C)C)c2)c1F]

In a 20 mL microwave vial was placed tert-butyl 2-(2-((3-bromo-5-formylbenzyl)oxy)phenyl)acetate (Intermediate 59B) (700 mg, 1.727 mmol), (R)-tert-butyl (1-(2-fluoro-3-(4,4,5,5-tetramethyl-1,3,2-dioxaborolan-2-yl)phenyl)ethyl)carbamate (Intermediate 27-B) (694 mg, 1.90 mmol), PdCl₂(dppf).CH₂Cl₂ adduct (70.5 mg, 0.086 mmol) in Acetonitrile (10 ml), K₃PO₄ (2M solution) (2.59 ml, 5.18 mmol) was added and the vial was sealed and heated in a microwave at 100° C. for 60 min. The reaction mixture was cooled to rt, the organic layer was filtered and purified by flash column (0-50% EtOAc/heptane) to give title compound. MS (ESI+) m/z 586.5 (M+Na).

### Intermediate 145

### (R)-tert-butyl 2-(2-((3′-(1-((tert-butoxycarbonyl)amino)ethyl)-5-((cyclopropylamino)methyl)-2′-fluoro-[1,1′-biphenyl]-3-yl)methoxy)phenyl)acetate

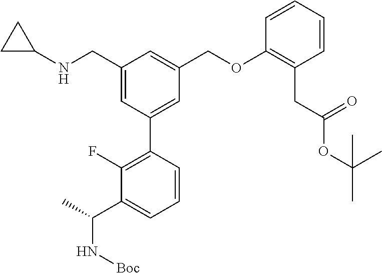 [SMILES: C[C@@H](NC(=O)OC(C)(C)C)c1cccc(-c2cc(CNC3CC3)cc(COc3ccccc3CC(=O)OC(C)(C)C)c2)c1F]

A solution of (R)-tert-butyl 2-(2-((3′-(1-((tert-butoxycarbonyl)amino)ethyl)-2′-fluoro-5-formyl-[1,1′-biphenyl]-3-yl)methoxy)phenyl)acetate (Intermediate 144) (96 mg, 0.17 mmol) in CF₃CH₂OH (2 ml) was stirred at 40° C. for 5 min. Cyclopropanamine (29.1 mg, 0.510 mmol) was added and stirred vigorously for 5 min. NaBH4 (19.29 mg, 0.510 mmol) was added and the reaction was stirred for 1 hour and then filtered and washed by DCM. The filtrate was concentrated and the residue was purified by flash colum (0-60% EtOAc/Heptane) to provide title compound. MS (ESI+) m/z 605.5 (M+H).

### Intermediate 146

The following intermediates were prepared with similar methods as described in Intermediate 145 using appropriate amine.

| | | MS (ESI+) |
|---|---|---|
| Intermediate | Structure/Chemical Name | m/z |
| 146-A | 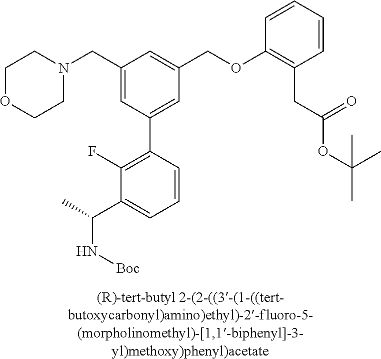 [SMILES: C[C@@H](NC(=O)OC(C)(C)C)c1cccc(-c2cc(COc3ccccc3CC(=O)OC(C)(C)C)cc(CN3CCOCC3)c2)c1F] | 635.6 (M + H) |
| 146-B | 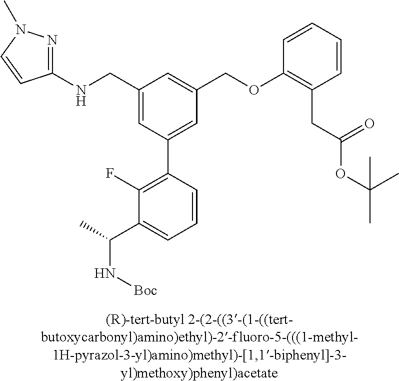 [SMILES: C[C@@H](NC(=O)OC(C)(C)C)c1cccc(-c2cc(CNc3ccn(C)n3)cc(COc3ccccc3CC(=O)OC(C)(C)C)c2)c1F] | 645.5 (M + H) |
| 146-C | 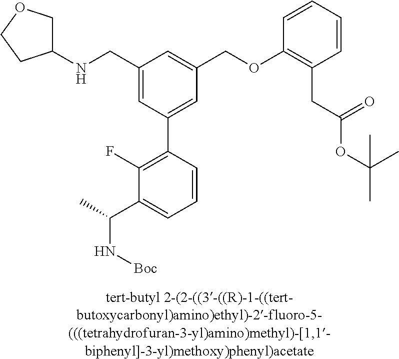 [SMILES: C[C@@H](NC(=O)OC(C)(C)C)c1cccc(-c2cc(CNC3CCOC3)cc(COc3ccccc3CC(=O)OC(C)(C)C)c2)c1F] | 635.6 (M + H) |
| 146-D | 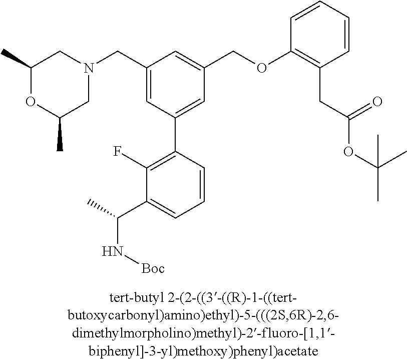 | 644.6 (M + H) |
| 146-E | 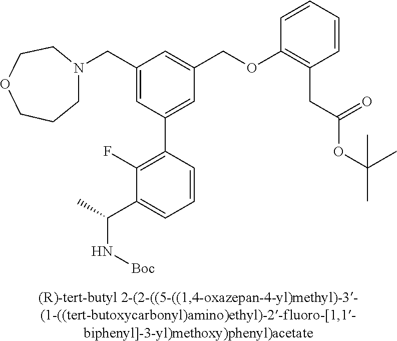 [SMILES: C[C@@H](NC(=O)OC(C)(C)C)c1cccc(-c2cc(COc3ccccc3CC(=O)OC(C)(C)C)cc(CN3CCCOCC3)c2)c1F] | 574.4 (M − tBu) |
| 146-F | 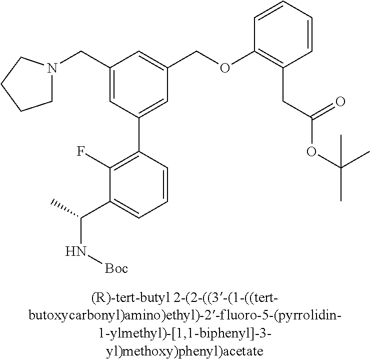 [SMILES: C[C@@H](NC(=O)OC(C)(C)C)c1cccc(-c2cc(COc3ccccc3CC(=O)OC(C)(C)C)cc(CN3CCCC3)c2)c1F] | 619.3 (M + H) |

### Intermediate 147

### (R)-tert-Butyl (1-(6-bromopyridin-2-yl)ethyl)carbamate

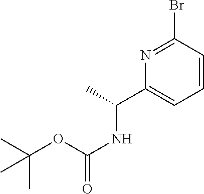 [SMILES: C[C@@H](NC(=O)OC(C)(C)C)c1cccc(Br)n1]

Boc-anhydride (1.162 mL, 5.00 mmol) was added in one portion to a DCM (15.64 mL) solution of (R)-1-(6-bromopyridin-2-yl)ethanamine hydrochloride (CAS #263718-60-1) (0.743 g, 3.13 mmol) and DIEA (1.147 mL, 6.57 mmol) at room temperature. After stirring for 45 minutes the reaction was purified directly by flash chromatography (0-50% EtOAc:Heptanes) to obtain title compound. MS (ESI+) m/z 300.9, 302. (M+H).

### Intermediate 148

### tert-Butyl 3-chloro-2-fluorobenzylcarbamate

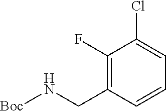 [SMILES: CC(C)(C)OC(=O)NCc1cccc(Cl)c1F]

(3-chloro-2-fluorophenyl)methanamine (CAS #72235-55-3)(0.205 mL, 1.629 mmol) was dissolved in DCM (8.15 mL) and added TEA (0.500 mL, 3.58 mmol) and Boc-anhydride (0.416 mL, 1.792 mmol) and stirred at room temperature for 2 hours. The reaction was diluted with water and then the DCM layer was removed, dried and concentrated and purified via FCC (0-50% EA:heptanes) to obtain the title compound. MS (ESI+) m/z 160.0 (M−Boc)⁺.

### Intermediate 149

### (R)-tert-butyl 2-(2-((3-bromo-5-((tetrahydrofuran-2-yl)methoxy)benzyl)oxy)phenyl)acetate

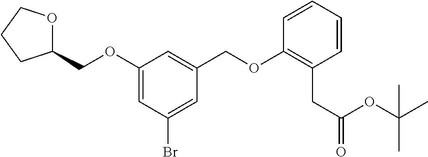 [SMILES: CC(C)(C)OC(=O)Cc1ccccc1OCc1cc(Br)cc(OC[C@H]2CCCO2)c1]

The title compound was synthesized in the same manner as Intermediate 73-C from (R)-(3-Bromo-5-((tetrahydrofuran-2-yl)methoxy)phenyl)methanol (Intermediate 73-B) and Intermediate 21. MS (ESI+) m/z 420.8, 422.8 (M−t-butyl)⁺.

### Intermediate 150

### (R)-tert-butyl 2-(2-((3-((tetrahydrofuran-2-yl)methoxy)-5-(4,4,5,5-tetramethyl-1,3,2-dioxaborolan-2-yl)benzyl)oxy)phenyl)acetate

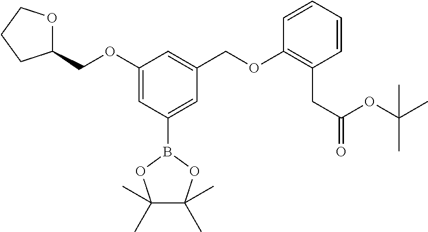 [SMILES: CC(C)(C)OC(=O)Cc1ccccc1OCc1cc(OC[C@H]2CCCO2)cc(B2OC(C)(C)C(C)(C)O2)c1]

The title compound was synthesized in the same manner as Intermediate 76 from (R)-tert-butyl 2-(2-((3-bromo-5-((tetrahydrofuran-2-yl)methoxy)benzyl)oxy)phenyl)acetate. MS (ESI+) m/z 469.1 (M−t-butyl)⁺.

### Intermediate 151

### tert-butyl 2-(2-((3-bromo-5-(bromomethyl)benzyl)oxy)phenyl)acetate

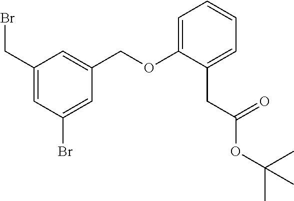 [SMILES: CC(C)(C)OC(=O)Cc1ccccc1OCc1cc(Br)cc(CBr)c1]

To Intermediate 59-A (400 mg, 0.982 mmol) in DCM (9 mL) under nitrogen was added PPh₃ (386 mg, 1.473 mmol) and CBr₄ (489 mg, 1.473 mmol). After 1 hour the reaction was purified directly by flash chromatography (0-40% EtOAc:Heptanes) to obtain title compound.

¹H NMR (400 MHz, DMSO-d₆) δ ppm 1.33 (s, 9H) 3.55 (s, 2H) 4.69 (s, 2H) 5.11 (s, 2H) 6.91 (td, J=7.40, 0.98 Hz, 1H) 7.01 (d, J=7.70 Hz, 1H) 7.17-7.28 (m, 2H) 7.51 (s, 1H) 7.59 (s, 1H) 7.64 (t, J=1.65 Hz, 1H).

### Intermediate 152

### tert-butyl 2-(2-((3-bromo-5-((triphenylphosphoranyl)methyl)benzyl)oxy)phenyl)acetate hydrobromide

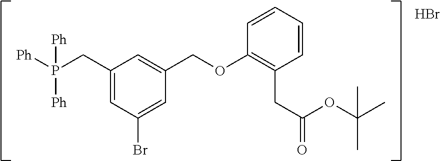

To tert-butyl 2-(2-((3-bromo-5-(bromomethyl)benzyl)oxy)phenyl)acetate (0.4148 g, 0.882 mmol) in toluene (2 mL) was added triphenylphosphine (0.243 g, 0.926 mmol) and the reaction was heated at 110° C. for 30 minutes. The reaction was cooled to room temperature and the solid was filtered off to obtain the title compound as the HBr salt. ¹H NMR (400 MHz, DMSO-d₆) δ ppm 7.87-7.96 (m, 3H) 7.63-7.79 (m, 12H) 7.59 (d, J=1.26 Hz, 1H) 7.18 (dt, J=7.26, 2.05 Hz, 2H) 7.06 (s, 1H) 6.99 (d, J=1.77 Hz, 1H) 6.87-6.94 (m, 1H) 6.84 (d, J=8.46 Hz, 1H) 5.16 (d, J=15.79 Hz, 2H) 4.95 (s, 2H) 3.46 (s, 2H) 1.31 (s, 9H).

### Intermediate 153

### tert-Butyl 2-(2-((3-bromo-5-(2-(fu ran-2-yl)vinyl)benzyl)oxy)phenyl)acetate

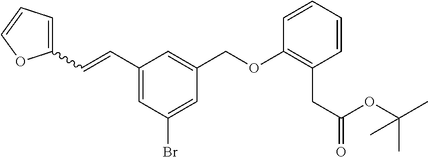 [SMILES: CC(C)(C)OC(=O)Cc1ccccc1OCc1cc(Br)cc(C=Cc2ccco2)c1]

To tert-butyl 2-(2-((3-bromo-5-((triphenylphosphoranyl)methyl)benzyl)oxy)phenyl)acetate hydrobromide (0.57 g, 0.776 mmol) in Water (7.7 mL) was added furan-2-carbaldehyde (0.129 ml, 1.552 mmol) and NaOH (0.047 g, 1.164 mmol) and then added about 1 mL of 4:1 ACN:water to help dissolve reactants. The reaction was heated at 100° C. for 10 minutes and then the reaction was cooled to room temperature, diluted with EA and brine. The organic layer was removed, dried and concentrated and absorbed onto silica to purify via fcc (0-50% EA:heptanes) to obtain the title compound. MS (ESI+) m/z 413.1, 415.1 (M−t-butyl)⁺.

### Intermediate 154

### 4-Benzyl-2-vinylmorpholine

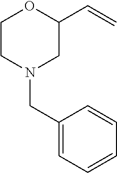

To allylpalladium chloride dimer (31.5 mg, 0.086 mmol) in DCM (18 mL) was added BINAP (230 mg, 0.369 mmol) and placed under nitrogen and the yellow solution was stirred at room temperature for 30 minutes. To the dark red solution was added 2-(benzylamino)ethanol (1.052 mL, 7.31 mmol) followed by (Z)-but-2-ene-1,4-diyl diacetate (1.254 mL, 7.94 mmol) and KF (0.95 g, 16.35 mmol) and the resulting red solution turned dark black/purple and was heated at 40° C. for 24 hours. The reaction was quenched with water and diluted with DCM. The DCM layer was removed, dried, concentrated and purified via FCC (0-100% EA:heptanes) to obtain the title compound. MS (ESI+) m/z 204.9 (M+H)⁺.

### Intermediate 155

### tert-Butyl 2-(2-((3-(2-(4-benzylmorpholin-2-yl)vinyl)-5-chlorobenzyl)oxy)phenyl)acetate

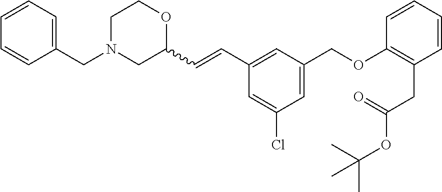 [SMILES: CC(C)(C)OC(=O)Cc1ccccc1OCc1cc(Cl)cc(C=CC2CN(Cc3ccccc3)CCO2)c1]

In a vial was placed tert-Butyl 2-(2-((3-bromo-5-chlorobenzyl)oxy)phenyl)acetate (0.342 g, 0.831 mmol) and 4-benzyl-2-vinylmorpholine (0.13 g, 0.640 mmol). Then tri-o-tolylphosphine (0.019 g, 0.064 mmol), triethylamine (0.267 mL, 1.919 mmol) and palladium(II) acetate (0.014 g, 0.064 mmol) were added and the vial was heated at 140° C. for 1 hour. The reaction was cooled to room temperature and diluted with EA and water. The EA layer was separated, dried, concentrated and absorbed onto silica to purify via FCC (0-70% EA:heptanes) to obtain the title compound. MS (ESI+) m/z 534.4, 536.4. (M+H).

### Intermediate 156

### tert-Butyl 2-(3-bromo-5-((2-(2-(tert-butoxy)-2-oxoethyl)phenoxy)methyl)styryl)pyrrolidine-1-carboxylate

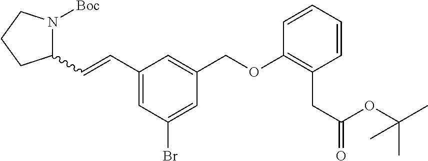 [SMILES: CC(C)(C)OC(=O)Cc1ccccc1OCc1cc(Br)cc(C=CC2CCCN2C(=O)OC(C)(C)C)c1]

The title compound was synthesized in the same manner as Example 155 from tert-butyl 2-(2-((3-bromo-5-((triphenylphosphoranyl)methyl)benzyl)oxy)phenyl)acetate hydrobromide (Intermediate 152) and tert-butyl 2-formylpyrrolidine-1-carboxylate (CAS #117625-90-8). MS (ESI+) m/z 415.95, 417.98 (M−t-butyl−Boc)⁺.

### Example 157

### (R)-tert-Butyl 2-(2-(5-((2-(2-(tert-butoxy)-2-oxoethyl)phenoxy)methyl)-3′-(1-((tert-butoxycarbonyl)amino)ethyl)-2′-fluoro-[1,1′-biphenyl]-3-yl)vinyl)pyrrolidine-1-carboxylate

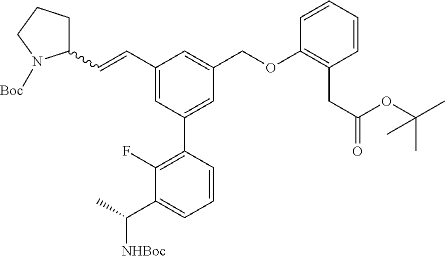 [SMILES: C[C@@H](NC(=O)OC(C)(C)C)c1cccc(-c2cc(C=CC3CCCN3C(=O)OC(C)(C)C)cc(COc3ccccc3CC(=O)OC(C)(C)C)c2)c1F]

The title compound was synthesized in the same manner as Example 141-A from tert-butyl 2-(3-bromo-5-((2-(2-(tert-butoxy)-2-oxoethyl)phenoxy)methyl)styryl)pyrrolidine-1-carboxylate and (R)-tert-butyl(1-(2-fluoro-3-(4,4,5,5-tetramethyl-1,3,2-dioxaborolan-2-yl)phenyl)ethyl)carbamate (Intermediate 27-B). MS (ESI+) m/z 631.6 (M−Boc)⁺.

### Intermediate 158

### tert-Butyl 2-(2-((3-bromo-5-(2-(pyrrolidin-2-yl)vinyl)benzyl)oxy)phenyl)acetate

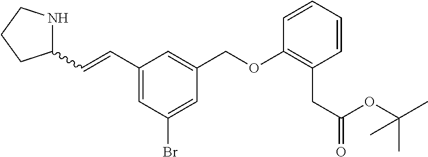 [SMILES: CC(C)(C)OC(=O)Cc1ccccc1OCc1cc(Br)cc(C=CC2CCCN2)c1]

To tert-butyl 2-(3-bromo-5-((2-(2-(tert-butoxy)-2-oxoethyl)phenoxy)methyl)styryl)pyrrolidine-1-carboxylate (Intermediate 156) (0.2577 g, 0.450 mmol) in t-butyl acetate (0.900 mL) was added sulfuric acid (0.036 mL, 0.675 mmol) and stirred for 3 hours at room temperature. Additional sulfuric acid (0.036 ml, 0.675 mmol) was added. The reaction was quenched with excess saturated solution (aq) of sodium bicarbonate, diluted with water and then EA and then the EA layer was removed, dried, concentrated to obtain an oil that was absorbed onto silica to purify via fcc (20-30% MeOH (containing 10% ammonium hydroxide):dcm) to obtain the title compound. MS (ESI+) m/z 471.9, 473.9 (M+H).

### Intermediate 159

### tert-Butyl 2-(2-((3-bromo-5-(2-(1-methylpyrrolidin-2-yl)vinyl)benzyl)oxy)phenyl)acetate

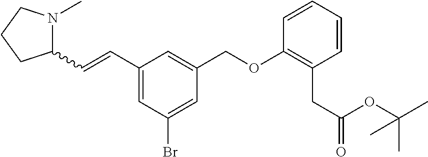 [SMILES: CN1CCCC1C=Cc1cc(Br)cc(COc2ccccc2CC(=O)OC(C)(C)C)c1]

To tert-butyl 2-(2-((3-bromo-5-(2-(pyrrolidin-2-yl)vinyl)benzyl)oxy)phenyl)acetate (0.1 g, 0.212 mmol) in MeOH (2.1 mL) was added paraformaldehyde (0.013 g, 0.423 mmol) and the reaction stirred for 45 minutes. Sodium cyanoborohydride (0.027 g, 0.423 mmol) was added and after 30 minutes the reaction was diluted with 1N NaOH (2 mL) and then diluted with EA. The EA layer was removed, dried, concentrated and absorbed onto silica to purify via fcc (0-100% EA:heptanes) to obtain the title compound. MS (ESI+) m/z 486.2, 488.2 (M+H).

### Intermediate 160

### tert-butyl 3-bromobenzyl(methyl)carbamate

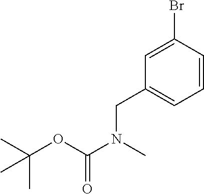 [SMILES: CN(Cc1cccc(Br)c1)C(=O)OC(C)(C)C]

Boc-anhydride (6.87 mL, 29.6 mmol) was added to a solution of 1-(3-bromophenyl)-N-methylmethanamine (3.7 g, 18.49 mmol) and DIEA (6.78 mL, 38.8 mmol) in CH₂Cl₂ (74 mL) at room temperature. After stirring for 16 hours the reaction mixture was poured into a saturated aqueous solution of NaHCO₃, the layers were separated and the aqueous layer extracted twice with CH₂Cl₂. The combined organic extracts were dried (phase separator) and purified by flash chromatography (0-20% EtOAc/c-hexane) to provide the title compound. ¹H NMR (400 MHz, DMSO-d₆) δ ppm 7.49 (d, J=7.95 Hz, 1H) 7.42 (s, 1H) 7.34 (t, J=7.75 Hz, 1H) 7.23 (d, J=7.55 Hz, 1H) 4.37 (s, 2H) 2.78 (s, 3H) 1.48 (s, 9H). MS (UPLC-MS): 244.0/246.4 [M−tBu+H]⁺.

### Intermediate 161

### tert-butyl methyl(3-(4,4,5,5-tetramethyl-1,3,2-dioxaborolan-2-yl)benzyl)carbamate

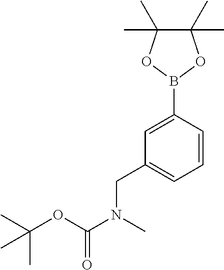 [SMILES: CN(Cc1cccc(B2OC(C)(C)C(C)(C)O2)c1)C(=O)OC(C)(C)C]

A solution of tert-butyl 3-bromobenzyl(methyl)carbamate (0.5 g, 1.67 mmol) in anhydrous dioxane (24.5 mL) was added under argon atmosphere to a vial containing bis(pinacolato)diboron (CAS #73183-34-3) (0.63 g, 2.50 mmol), potassium acetate (0.65 g, 6.66 mmol) and PdCl₂(dppf).CH₂Cl₂ adduct (CAS #95464-05-4) (0.136 g, 0.167 mmol). The vial was capped and the reaction heated at 80° C. for 36 hours. The reaction was cooled, diluted with EtOAc and filtered over a pad of Celite. The crude mixture was concentrated and purified by flash chromatography (0-30% EtOAc/c-hexane) to provide the title product. TLC, R_{f} (c-hexane/EtOAc 1/1)=0.77. MS (UPLC-MS): 292.2 [M−tBu+H]+, 348.3 [M+H]+, 365.3 [M+18]+. Rt (UPLC-MS, method C): 1.42 min.

### Intermediate 162

### methyl 2-(2-((3′-(((tert-butoxycarbonyl)(methyl)amino)methyl)-[1,1′-biphenyl]-3-yl)methoxy)phenyl)acetate

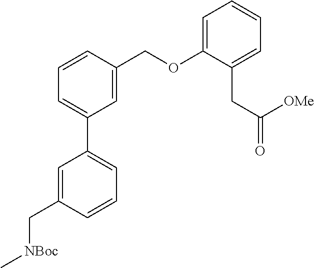 [SMILES: COC(=O)Cc1ccccc1OCc1cccc(-c2cccc(CN(C)C(=O)OC(C)(C)C)c2)c1]

A mixture of methyl 2-(2-((3-bromobenzyl)oxy)phenyl)acetate (60 mg, 0.179 mmol), tert-butyl methyl(3-(4,4,5,5-tetramethyl-1,3,2-dioxaborolan-2-yl)benzyl)carbamate (62.2 mg, 0.179 mmol), K₃PO₄ (2N in water, 0.27 mL, 0.537 mmol) and PdCl₂(dppf).CH₂Cl₂ adduct (CAS #95464-05-4) (7.31 mg, 8.95 μmol) in CH₃CN (2.23 mL) was heated to 120° C. for 40 min in the microwave, then cooled, diluted with EtOAc and filtered through a pad of Celite. The crude reaction mixture was concentrated and treated with a metal scavenger to remove residual palladium according to the following protocol: the mixture was diluted with THF (4 mL) SiliaMetS®Thiol (Silicycle, particle size: 40-63 μm, loading 1.39 mmol/g, 4 equiv. for 1 equiv of Pd complex used in the reaction) (25.8 mg, 0.036 mmol) was added and the mixture was shaked at 40° C. for 1 hour. The mixture was filtered, washed with THF and the filtrate concentrated and purified by flash column chromatography on silica gel (0-20% EtOAc/c-hexane) to give the title compound. MS (UPLC-MS): 420.2 [M−tBu+H]+, 476.2 [M+H]+, 493.4 [M+18]+, 474.2 [M−H]−. Rt (UPLC-MS, method C): 1.46 min.

### Intermediate 163

### methyl 2-(2-((3′-((methylamino)methyl)-[1,1′-biphenyl]-3-yl)methoxy)phenyl)acetate

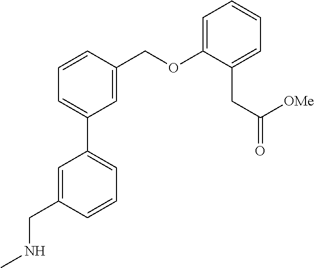 [SMILES: CNCc1cccc(-c2cccc(COc3ccccc3CC(=O)OC)c2)c1]

Methyl 2-(2-((3′-(((tert-butoxycarbonyl)(methyl)amino)methyl)-[1,1′-biphenyl]-3-yl)methoxy)phenyl)acetate (60 mg, 0.126 mmol) was stirred in HCl (4N in dioxane, 0.315 mL, 1.26 mmol) and dioxane (2 mL) at room temperature for 45 min, then freeze dried and lyophilized to provide the title compound as HCl salt. MS (UPLC-MS): 376.2 [M+H]+, 751.5 [2M+H]+. Rₜ (UPLC-MS, method C): 0.90 min.

### Intermediate 164

### tert-butyl 2-(2-((3-bromo-4-cyanobenzyl)oxy)phenyl)acetate

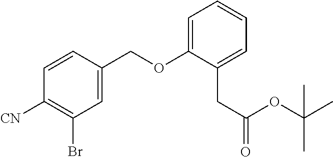

To a solution of tert-butyl 2-(2-hydroxyphenyl)acetate (180 mg, 0.864 mmol) in THF (6 mL) under nitrogen atmosphere were successively added triphenylphosphine (249 mg, 0.951 mmol) and 3-bromo-4-cyanobenzylalcohol (202 mg, 0.951 mmol). The solution was cooled to 0° C., diisopropyl azodicarboxylate (0.185 mL, 0.951 mmol) was added dropwise and the mixture was stirred at 0° C. and allowed to reach RT overnight. The reaction mixture was poured into a sat. aq. NaHCO₃ solution and extracted with EtOAc (2×). The combined organic layers were dried (phase separator), concentrated and the crude mixture was purified by flash chromatography (0-100% AcOEt/c-hexane) to give the title compound. TLC, Rf (c-hexane/EtOAc 1:1): 0.85. MS (UPLC-MS): 402.2/404.0 [M+H]+, 419.2/421.1 [M+18]+, 400.1/402.2 [M−H]−, 446.2/448.1 [M+HCOO]−. Rₜ (HPLC, method E): 2.69 min.

### Intermediate 165

### tert-Butyl 2-(2-((3′-(((tert-butoxycarbonyl)amino)methyl)-6-cyano-[1,1′-biphenyl]-3-yl)methoxy)phenyl)acetate

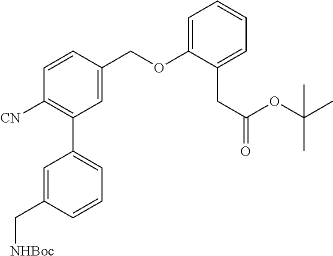

The title compound was prepared using the same protocol as described for the preparation of methyl 2-(2-((3′-(((tert-butoxycarbonyl)(methyl)amino)methyl)-[1,1′-biphenyl]-3-yl)methoxy)phenyl)acetate using tert-butyl 2-(2-((3-bromo-4-cyanobenzyl)oxy)phenyl)acetate. TLC, R_{f}(c-hexane/EtOAc 1:1): 0.90. MS (UPLC-MS): 529.4 [M+H]+, 546.4 [M+18]+, 1074.8 [2M+18]+, 573.3 [M+HCOO]−, 1101.8 [2M+HCOO-]−. Rt (HPLC, method E): 2.84 min.

### Intermediate 166

### tert-butyl 2-(2-((3-bromobenzyl)oxy)phenyl)propanoate

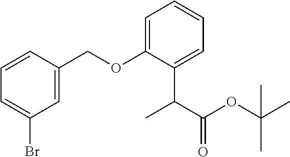 [SMILES: CC(C(=O)OC(C)(C)C)c1ccccc1OCc1cccc(Br)c1]

tert-Butyl 2-(2-((3-bromobenzyl)oxy)phenyl)acetate (60 mg, 0.16 mmol) was added at 0° C. to a solution of NaH (60% in mineral oil, 12.7 mg, 0.32 mmol) in DMF (1 mL) under nitrogen atmosphere. The mixture was stirred for 20 min at 0° C., iodomethane (20 μL, 0.32 mmol) was added and the reaction mixture further stirred for 6 hours while slowly allowed to reach RT. The mixture was poured into water and extracted with EtOAc (×2). The combined organic extracts were dried (phase separator), concentrated and the crude mixture was used without purification in the next step. MS (UPLC-MS): 391.1/393.1 [M+H]+, 408.2/410.2 [M+18]+, 335.1/337.1 [M+H−tBu]+.

### Intermediate 167

### tert-butyl 2-(2-((3′-(((tert-butoxycarbonyl)amino)methyl)-[1,1′-biphenyl]-3-yl)methoxy)phenyl)propanoate

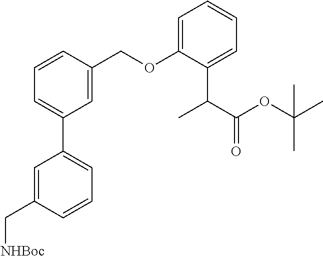 [SMILES: CC(C(=O)OC(C)(C)C)c1ccccc1OCc1cccc(-c2cccc(CNC(=O)OC(C)(C)C)c2)c1]

The title compound was prepared using the same protocol as described for the preparation of methyl 2-(2-((3′-(((tert-butoxycarbonyl)(methyl)amino)methyl)-[1,1′-biphenyl]-3-yl)methoxy)phenyl)acetate using tert-butyl 2-(2-((3-bromobenzyl)oxy)phenyl)propanoate (65 mg, 0.16 mmol), 3-(N-Boc-aminomethyl)phenylboronic acid pinacol ester (79 mg, 0.24 mmol), K₃PO₄ (2N in water, 0.237 mL, 0.47 mmol) and PdCl₂(dppf).CH₂Cl₂ adduct (6.44 mg, 7.89 μmol) in acetonitrile (2 mL). TLC, R_{f}(c-hexane/EtOAc 4:1): 0.45. MS (UPLC-MS): 518.4 [M+H]+, 535.4 [M+18]+, 562.4 [M+HCOO]−.

### Intermediate 168

### methyl 2-(4-bromo-2-hydroxyphenyl)acetate

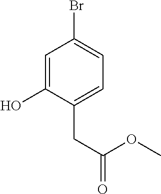

To a solution of 4-bromo-2-hydroxyphenyl acetic acid (1.08 g, 4.67 mmol) in MeOH (10 mL) at 0° C. under an argon atmosphere was added dropwise thionyl chloride (0.682 mL, 9.35 mmol). The reaction was slowly allowed to reach RT and stirred at RT for 3 hours. The reaction mixture was concentrated to dryness to give the title compound which was used without purification in the next step. TLC, R_{f}(c-hexane/EtOAc 3:7): 0.85. MS (UPLC-MS): 243.0/245.0 [M+H]+. Rₜ (HPLC, method E): 1.71 min.

### Intermediate 169

### methyl 2-(4-bromo-2-((3′-(((tert-butoxycarbonyl)amino)methyl)-[1,1′-biphenyl]-3-yl)methoxy)phenyl)acetate

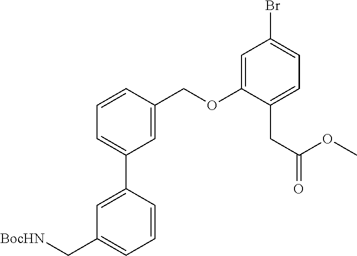 [SMILES: COC(=O)Cc1ccc(Br)cc1OCc1cccc(-c2cccc(CNC(=O)OC(C)(C)C)c2)c1]

The title compound was prepared using the same protocol as described for the preparation of tert-butyl 2-(2-((3-bromo-4-cyanobenzyl)oxy)phenyl)acetate using methyl 2-(4-bromo-2-hydroxyphenyl)acetate and tert-butyl ((3′-(hydroxymethyl)-[1,1′-biphenyl]-3-yl)methyl)carbamate. The crude mixture was purified by flash chromatography (0-80% AcOEt/c-hexane) to give the title compound. TLC, R_{f}(c-hexane/EtOAc 8:2): 0.4. MS (UPLC-MS): 540.2-542.2 [M+H]+, 584.1-586.1 [M+HCOO]−. Rₜ (HPLC, method E): 2.85 min.

### Intermediate 170

### methyl 2-(2-((3′-(((tert-butoxycarbonyl)amino)methyl)-[1,1′-biphenyl]-3-yl)methoxy)-4-ethylphenyl)acetate

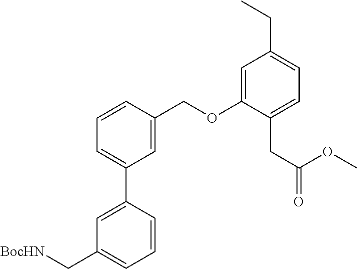 [SMILES: CCc1ccc(CC(=O)OC)c(OCc2cccc(-c3cccc(CNC(=O)OC(C)(C)C)c3)c2)c1]

To a mixture of methyl 2-(4-bromo-2-((3′-(((tert-butoxycarbonyl)amino)methyl)-[1,1′-biphenyl]-3-yl)methoxy)phenyl)acetate (80 mg, 0.148 mmol), ethylboronic acid (43.7 mg, 0.592 mmol) and PdCl₂(dppf).CH₂Cl₂ adduct (6.04 mg, 7.40 μmol) under argon atmosphere were added toluene (1.1 mL), dioxane (0.275 mL) and NaHCO₃ (2N in water, 0.52 mL, 1.04 mmol). The resulting suspension was stirred and heated at 85° C. for 16 hours. The reaction mixture was allowed to cool to RT and filtered through a pad of Celite. Water and EtOAc were added, the layers were separated and the aqueous layer was extracted with EtOAc. The combined organic extracts were dried (phase separator) and concentrated. The crude residue was treated with a metal scavenger using the same protocol as described in the preparation of methyl 2-(2-((3′-(((tert-butoxycarbonyl)(methyl)amino)methyl)-[1,1′-biphenyl]-3-yl)methoxy)phenyl)acetate and purified by flash chromatography on silica gel (0-20% AcOEt/c-hexane) to give the title compound. TLC, R_{f} (c-hexane/EtOAc 9:1): 0.35. MS (UPLC-MS): 490.3 [M+H]+, 507.3 [M+18]+, 488.3 [M−H]−, 534.3 [M+HCOO]−. Rₜ (HPLC, method E): 2.89 min.

### Intermediate 171

### 2-(2-((3′-(((tert-butoxycarbonyl)amino)methyl)-[1,1′-biphenyl]-3-yl)methoxy)-4-ethylphenyl)acetic acid

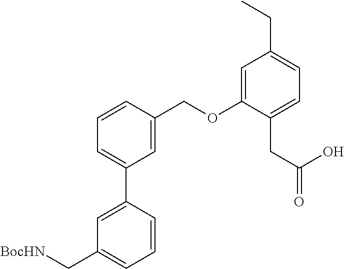 [SMILES: CCc1ccc(CC(=O)O)c(OCc2cccc(-c3cccc(CNC(=O)OC(C)(C)C)c3)c2)c1]

To a mixture of methyl 2-(2-((3′-(((tert-butoxycarbonyl)amino)methyl)-[1,1′-biphenyl]-3-yl)methoxy)-4-ethylphenyl)acetate (46 mg, 0.094 mmol) in MeOH (0.85 mL) and water (85 μL) NaOH (1N in water, 470 μL, 0.470 mmol) was added and the reaction mixture was stirred at room temperature until completion. The reaction mixture was acidified with 1N HCl, CH₂Cl₂ and water were added, the layers were separated and the aqueous layer was extracted with CH₂Cl₂ (×2). The combined organic extracts were dried (phase separator) and concentrated to give the title compound which was used without purification in the next step. MS (UPLC-MS): 476.3 [M+H]+, 968.6 [2M+18]+, 474.2 [M−H]−, 949.5[2M−H]−. Rt (HPLC, method E): 2.69 min.

### Intermediate 172

### methyl 2-(2-((3′-(((tert-butoxycarbonyl)amino)methyl)-[1,1′-biphenyl]-3-yl)methoxy)-4-cyanophenyl)acetate

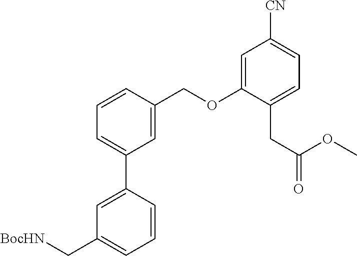 [SMILES: COC(=O)Cc1ccc(C#N)cc1OCc1cccc(-c2cccc(CNC(=O)OC(C)(C)C)c2)c1]

A mixture of methyl 2-(4-bromo-2-((3′-(((tert-butoxycarbonyl)amino)methyl)-[1,1′-biphenyl]-3-yl)methoxy)phenyl)acetate (178 mg, 0.329 mmol), zinc cyanide (38.7 mg, 0.329 mmol), Pd(PPh₃)₄ (114 mg, 0.099 mmol) under argon atmosphere in DMF (2.2 mL) was microwaved at 120° C. for 40 min. The reaction mixture was filtered through a pad of Celite. EtOAc and an aq. sat. solution of NaHCO₃ were added, the layers were separated and the aqueous layer was extracted with EtOAc (×2). The combined organic extracts were washed with brine, dried (phase separator) and concentrated. The crude residue was treated with a metal scavenger using the same protocol as described in the preparation of methyl 2-(2-((3′-(((tert-butoxycarbonyl)(methyl)amino)methyl)-[1,1′-biphenyl]-3-yl)methoxy)phenyl)acetate and purified by flash chromatography on silica gel (0-30% AcOEt/c-hexane) to give the title compound. TLC, R_{f} (c-hexane/EtOAc 8:2): 0.30. MS (UPLC-MS): 487.3 [M+H]+, 990.5 [2M+18]+, 485.2 [M−H]−, 531.3 [M+HCOO]−, 1017.5 [2M+HCOO]−. Rt (HPLC, method E): 2.61 min.

### Intermediate 173

### methyl 2-(2-((3′-(((tert-butoxycarbonyl)amino)methyl)-[1,1′-biphenyl]-3-yl)methoxy)-4-carbamoylphenyl)acetate

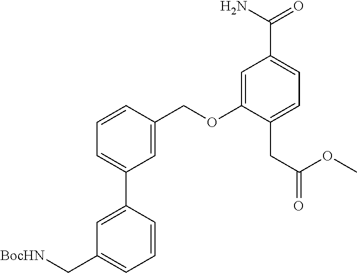 [SMILES: COC(=O)Cc1ccc(C(N)=O)cc1OCc1cccc(-c2cccc(CNC(=O)OC(C)(C)C)c2)c1]

A mixture of methyl 2-(2-((3′-(((tert-butoxycarbonyl)amino)methyl)-[1,1′-biphenyl]-3-yl)methoxy)-4-cyanophenyl)acetate (60 mg, 0.123 mmol), acetamide (30.6 mg, 0.518 mmol), PdCl₂ (2.2 mg, 0.012 mmol) in THF (0.18 mL) and water (0.06 mL) was stirred at RT for 5 hours. EtOAc and water were added, the layers were separated and the aqueous layer was extracted with EtOAc (×2). The combined organic extracts were washed with brine, dried (phase separator) and concentrated. The crude residue was treated with a metal scavenger using the same protocol as described in the preparation of methyl 2-(2-((3′-(((tert-butoxycarbonyl)(methyl)amino)methyl)-[1,1′-biphenyl]-3-yl)methoxy)phenyl)acetate to give after filtration of the scavenger and concentration the title compound which was used without purification in the next step. MS (UPLC-MS): 505.3 [M+H]+, 522.3 [M+18]+, 549.3 [M+HCOO]−, 1053.5 [2M+HCOO]−. Rt (HPLC, method E): 2.25 min.

### Intermediate 174

### methyl 2-(4-(aminomethyl)-2-((3′-(((tert-butoxycarbonyl)amino)methyl)-[1,1′-biphenyl]-3-yl)methoxy)phenyl)acetate

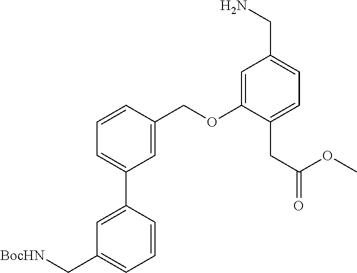 [SMILES: COC(=O)Cc1ccc(CN)cc1OCc1cccc(-c2cccc(CNC(=O)OC(C)(C)C)c2)c1]

To a mixture of methyl 2-(2-((3′-(((tert-butoxycarbonyl)amino)methyl)-[1,1′-biphenyl]-3-yl)methoxy)-4-cyanophenyl)acetate (79 mg, 0.162 mmol) and CoCl₂.6 H₂O (77 mg, 0.325 mmol) in MeOH (2 mL) under a nitrogen atmosphere was added portion wise at 0° C. NaBH₄ (30.7 mg, 0.812 mmol). The reaction mixture was stirred at 0° C. for 30 min and at RT for 16 hours. The reaction mixture was cooled to 0° C., 3 equivalents of NaBH₄ were added and the mixture was further stirred at RT for 21 hours. EtOAc and an aq. sat. solution of NaHCO₃ were added, the layers were separated and the aqueous layer was extracted with EtOAc. The combined organic extracts were dried (phase separator) and concentrated to give the title compound which was used without purification in the next step. MS (UPLC-MS): 491.3 [M+H]+, 981.6 [2M+H]+, 489.3 [M−H]−, 535.2 [M+HCOO]−, 1025.5 [2M+HCOO]−. Rt (HPLC, method E): 2.01 min.

### Intermediate 175

### methyl 2-(4-(acetamidomethyl)-2-((3′-(((tert-butoxycarbonyl)amino)methyl)-[1,1′-biphenyl]-3-yl)methoxy)phenyl)acetate

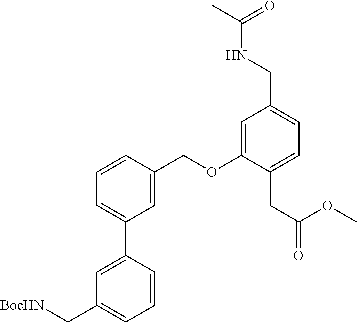 [SMILES: COC(=O)Cc1ccc(CNC(C)=O)cc1OCc1cccc(-c2cccc(CNC(=O)OC(C)(C)C)c2)c1]

To a mixture of methyl 2-(4-(aminomethyl)-2-((3′-(((tert-butoxycarbonyl)amino)methyl)-[1,1′-biphenyl]-3-yl)methoxy)phenyl)acetate (79 mg, 0.161 mmol) and triethylamine (0.045 mL, 0.322 mmol) in CH₂Cl₂ (0.65 mL) was added acetyl chloride (0.023 mL, 0.322 mmol) and the reaction mixture was stirred at RT for 2 hours. CH₂Cl₂ and water were added, the layers were separated and the aqueous layer was extracted with CH₂Cl₂ (×2). The combined organic extracts were dried (phase separator) and concentrated. The crude residue was purified by flash chromatography on silica gel (0-100% AcOEt/c-hexane) to give the title compound. TLC, R_{f} (EtOAc): 0.4. MS (UPLC-MS): 533.3 [M+H]+, 550.3 [M+18]+, 1082.7 [2M+18]+, 577.3 [M+HCOO]−, 1109.6 [2M+HCOO]−. Rₜ (HPLC, method E): 2.32 min.

### Intermediate 176

### methyl 2-(2-((3′-(((tert-butoxycarbonyl)amino)methyl)-[1,1′-biphenyl]-3-yl)methoxy)-4-(ethylamino)phenyl)acetate

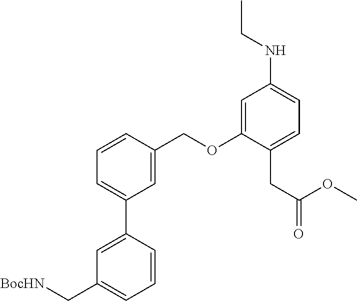 [SMILES: CCNc1ccc(CC(=O)OC)c(OCc2cccc(-c3cccc(CNC(=O)OC(C)(C)C)c3)c2)c1]

To a mixture of methyl 2-(4-bromo-2-((3′-(((tert-butoxycarbonyl)amino)methyl)-[1,1′-biphenyl]-3-yl)methoxy)phenyl)acetate (60 mg, 0.111 mmol), BrettPhos precatalyst (4.43 mg, 5.55 μmol), BrettPhos (2.98 mg, 5.55 μmol) and Cs₂CO₃ (72.3 mg, 0.222 mmol) were added under an argon atmosphere toluene (1.1 mL) and ethylamine (2M in THF, 0.056 mL, 0.111 mmol). The reaction mixture was stirred and heated at 90° C. for 19 hours. To allow completion of the reaction BrettPhos precatalyst (4.43 mg, 5.55 μmol), BrettPhos (2.98 mg, 5.55 μmol) and ethylamine (2M in THF, 0.056 mL, 0.111 mmol) were added again and the reaction mixture was further stirred for 70 hours. The reaction mixture was allowed to cool to RT and filtered through a pad of Celite. The solvents were evaporated and the crude residue was treated with a metal scavenger using the same protocol as described in the preparation of methyl 2-(2-((3′-(((tert-butoxycarbonyl)(methyl)amino)methyl)-[1,1′-biphenyl]-3-yl)methoxy)phenyl)acetate and purified by flash chromatography on silica gel (0-30% AcOEt/c-hexane) to give the title compound. MS (UPLC-MS): 505.3 [M+H]+, 1009.6 [2M+H]+, 549.3 [M+HCOO]−, 1053.6 [2M+HCOO]−. Rₜ (HPLC, method E): 2.09 min.

### Intermediate 177

### ethyl 2-(5-bromo-2-((3′-(((tert-butoxycarbonyl)amino)methyl)-[1,1′-biphenyl]-3-yl)methoxy)phenyl)acetate

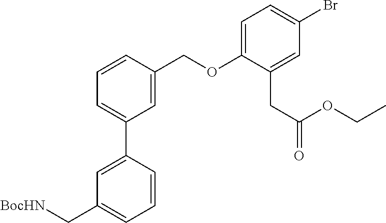 [SMILES: CCOC(=O)Cc1cc(Br)ccc1OCc1cccc(-c2cccc(CNC(=O)OC(C)(C)C)c2)c1]

The title compound was prepared using the same protocol as described for the preparation of tert-butyl 2-(2-((3-bromo-4-cyanobenzyl)oxy)phenyl)acetate using ethyl 2-(5-bromo-2-hydroxyphenyl)acetate and tert-butyl ((3′-(hydroxymethyl)-[1,1′-biphenyl]-3-yl)methyl)carbamate. The crude mixture was purified by flash chromatography (0-80% AcOEt/c-hexane) to give the title compound. TLC, R_{f} (c-hexane/EtOAc 8:2): 0.45. MS (UPLC-MS): 554.2-556.2 [M+H]+, 571.2-576.2 [M+18]+, 598.2-600.3 [M+HCOO]−. Rₜ (HPLC, method E): 2.94 min.

### Intermediate 178

### ethyl 2-(2-((3′-(((tert-butoxycarbonyl)amino)methyl)-[1,1′-biphenyl]-3-yl)methoxy)-5-cyanophenyl)acetate

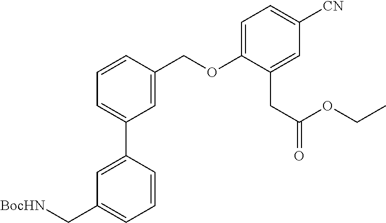 [SMILES: CCOC(=O)Cc1cc(C#N)ccc1OCc1cccc(-c2cccc(CNC(=O)OC(C)(C)C)c2)c1]

The title compound was prepared using the same protocol as described for the preparation of methyl 2-(2-((3′-(((tert-butoxycarbonyl)amino)methyl)-[1,1′-biphenyl]-3-yl)methoxy)-4-cyanophenyl)acetate using ethyl 2-(5-bromo-2-((3′-(((tert-butoxycarbonyl)amino)methyl)-[1,1′-biphenyl]-3-yl)methoxy)phenyl)acetate. TLC, R_{f}(c-hexane/EtOAc 8:2): 0.3. MS (UPLC-MS): 501.3 [M+H]+, 518.3 [M+18H]+, 1018.6 [2M+18H]+, 555.3 [M+HCOO]−, 1045.5 [2M+HCOO]−. Rₜ (HPLC, method E): 2.67 min.

### Intermediate 179

### ethyl 2-(2-((3′-(((tert-butoxycarbonyl)amino)methyl)-[1,1′-biphenyl]-3-yl)methoxy)-5-carbamoylphenyl)acetate

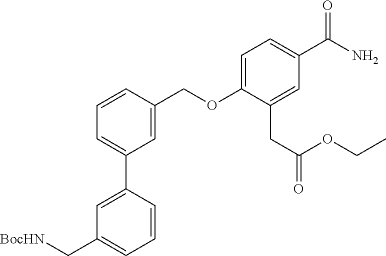 [SMILES: CCOC(=O)Cc1cc(C(N)=O)ccc1OCc1cccc(-c2cccc(CNC(=O)OC(C)(C)C)c2)c1]

The title compound was prepared using the same protocol as described for the preparation of methyl 2-(2-((3′-(((tert-butoxycarbonyl)amino)methyl)-[1,1′-biphenyl]-3-yl)methoxy)-4-carbamoylphenyl)acetate using ethyl 2-(2-((3′-(((tert-butoxycarbonyl)amino)methyl)-[1,1′-biphenyl]-3-yl)methoxy)-5-cyanophenyl)acetate. TLC, R_{f} (c-hexane/EtOAc 8:2): 0.3. MS (UPLC-MS): 519.3 [M+H]+, 536.3 [M+18]+, 1037.7 [2M+H]+, 563.3 [M+HCOO]−, 1081.6 [2M+HCOO]−. Rₜ (HPLC, method E): 2.32 min.

### Intermediate 180

### methyl 2-amino-2-(2-((3-chlorobenzyl)oxy)phenyl)acetate

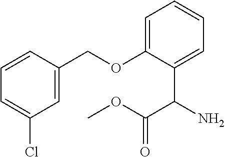 [SMILES: COC(=O)C(N)c1ccccc1OCc1cccc(Cl)c1]

To a suspension of (S)-2-amino-2-(2-((3-chlorobenzyl)oxy)phenyl)acetic acid (ChemSpider ID: 27466595) (400 mg, 1.22 mmol) in MeOH (20 mL) was carefully added thionyl chloride (0.178 mL, 2.44 mmol) and the reaction mixture was slowly heated to 50° C. and stirred for 16 hours. The reaction was allowed to cool to RT, thionyl chloride (0.178 mL, 2.44 mmol) was again carefully added and the mixture stirred at 50° C. for 5 hours. The reaction mixture was concentrated to dryness to afford the title compound as a yellow powder. MS (UPLC-MS): 306.1/308.1 [M+H]+, 311.3/613.2 [2M+H]+. Rₜ (HPLC, method E): 1.61 min.

### Intermediate 181

### methyl 2-acetamido-2-(2-((3-chlorobenzyl)oxy)phenyl)acetate

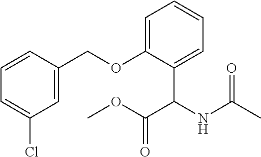 [SMILES: COC(=O)C(NC(C)=O)c1ccccc1OCc1cccc(Cl)c1]

To a solution of methyl 2-amino-2-(2-((3-chlorobenzyl)oxy)phenyl)acetate (150 mg, 0.394 mmol) in CH₂Cl₂ (6 mL) were added successively DIPEA (0.344 mL, 1.97 mmol), acetic acid (0.027 mL, 0.473 mmol) and propylphosphonic anhydride (0.423 mL, 0.710 mmol). The reaction mixture was stirred at 23° C. for 16 hours. CH₂Cl₂ and an aq. sat. NaHCO₃ solution were added, the layers were separated and the aqueous layer was extracted twice with CH₂Cl₂. The combined organic extracts were dried (phase separator) and concentrated. The crude mixture was purified by flash chromatography (0-50% AcOEt/c-hexane) to give the title compound. TLC, R_{f} (c-hexane/EtOAc 1:1): 0.4. MS (UPLC-MS): 348.2/350.1 [M+H]+, 346.2/347.2 [M−H]−, 392.2/394.3 [M+HCOO]−. Rₜ (HPLC, method E): 2.01 min.

### Intermediate 182

### methyl 2-acetamido-2-(2-((3′-(((tert-butoxycarbonyl)amino)methyl)-[1,1′-biphenyl]-3-yl)methoxy)phenyl)acetate

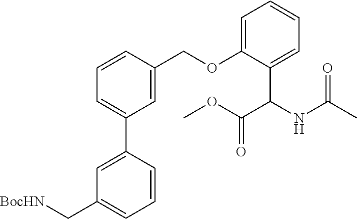 [SMILES: COC(=O)C(NC(C)=O)c1ccccc1OCc1cccc(-c2cccc(CNC(=O)OC(C)(C)C)c2)c1]

A microwave vial was charged under argon atmosphere with methyl 2-acetamido-2-(2-((3-chlorobenzyl)oxy)phenyl)acetate (40 mg, 0.115 mmol), 3-(N-Boc aminomethyl)phenylboronic acid pinacol ester (57.5 mg, 0.17 mmol), Pd₂dba₃ (2.1 mg, 2.3 μmol), X-Phos (4.39 mg, 9.2 μmol), CsF (61.1 mg, 0.40 mmol) and dioxane (3.2 mL). The vial was heated at 120° C. under microwave irradiations for 1 hour. Pd₂dba₃ (2.1 mg, 2.3 μmol) and X-Phos (4.39 mg, 9.2 μmol) were added again and the reaction mixture was further heated under microwave irradiations at 120° C. for 30 min. The reaction mixture was diluted with EtOAc, aq. sat. NaHCO₃ was added, the layers were separated and the aqueous layer was extracted twice with EtOAc. The combined organic extracts were dried (phase separator) and concentrated. The crude residue was treated with a metal scavenger using the same protocol as described in the preparation of methyl 2-(2-((3′-(((tert-butoxycarbonyl)(methyl)amino)methyl)-[1,1′-biphenyl]-3-yl)methoxy)phenyl)acetate and purified by flash chromatography on silica gel (0-50% AcOEt/c-hexane) to give the title compound. TLC, R_{f}(EtOAc): 0.8. MS (UPLC-MS): 519.3 [M+H]+, 563.3 [M+HCOO]−. Rₜ (HPLC, method E): 2.37 min.

### Intermediate 183

### 2-acetamido-2-(2-((3′-(((tert-butoxycarbonyl)amino)methyl)-[1,1′-biphenyl]-3-yl)methoxy)phenyl)acetic acid

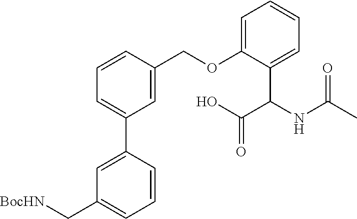 [SMILES: CC(=O)NC(C(=O)O)c1ccccc1OCc1cccc(-c2cccc(CNC(=O)OC(C)(C)C)c2)c1]

To methyl 2-acetamido-2-(2-((3′-(((tert-butoxycarbonyl)amino)methyl)-[1,1′-biphenyl]-3-yl)methoxy)phenyl)acetate (45 mg, 0.07 mmol) in MeOH (1.4 mL) and CH₃CN (1.4 mL) NaOH (1N in water, 721 μL, 0.72 mmol) was added and the reaction was stirred at room temperature for 16 hours. MeOH was partially evaporated and CH₂Cl₂ and a 10% aq. solution of KHSO₄ were added. The layers were separated and the aqueous layer was extracted with CH₂Cl₂ (×3). The combined organic extracts were dried (phase separator) and concentrated to give the title compound which was used without purification in the next step. MS (UPLC-MS): 505.2 [M+H]+, 503.2 [M−H]−, 1007.5 [2M−H]−. Rt (HPLC, method E): 2.20 min.

### Example 1

### Example 1-A

### 3′-(((tert-Butoxycarbonyl)amino)methyl)-[1,1′-biphenyl]-3-carboxylic acid

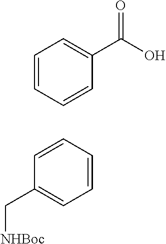

A degassed mixture of 3-bromobenzoic acid (CAS #585-76-2) (1 g, 4.97 mmol), 3-((tert-butoxycarbonylamino)methyl)phenylboronic acid (CAS #832114-05-3) (1.249 g, 4.97 mmol), PdCl₂(dppf).CH₂Cl₂ adduct (CAS #95464-05-4) (0.203 g, 0.249 mmol), and 2M aq. K₃PO₄ (7.46 ml, 14.92 mmol) in CH₃CN (25 mL) was heated at 90° C. for 2 hr. The reaction mixture was diluted with EtOAc and washed with water. The aqueous layer was extracted with EtOAc. The combined organics were washed with brine, dried (Na₂SO₄) and concentrated. The residue was purified by FCC (EtOAc-heptane 0-100%) to provide the title compound. MS (ESI−) m/z 326.2 (M−H).

### Example 1-B

### Methyl 2-(2-(3′-(((tert-butoxycarbonyl)amino)methyl)-[1,1′-biphenyl]-3-ylcarboxamido)phenyl)acetate

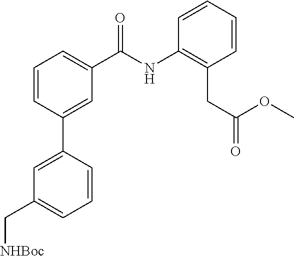 [SMILES: COC(=O)Cc1ccccc1NC(=O)c1cccc(-c2cccc(CNC(=O)OC(C)(C)C)c2)c1]

TEA (0.255 mL, 1.833 mmol) was added to a mixture of 3′-(((tert-butoxycarbonyl)amino)methyl)-[1,1′-biphenyl]-3-carboxylic acid (200 mg, 0.611 mmol) and HATU (256 mg, 0.672 mmol) in DMF at 23° C. After 15 min, methyl 2-(2-aminophenyl)acetate hydrochloride (CAS #35613-44-6) (130 mg, 0.611 mmol) was added and the resulting mixture was stirred at room temperature overnight. The resulting mixture was partitioned between with EtOAc and H₂O. The layers were separated and the aqueous layer was extracted with 4:1 EtOAc/heptane. The combined organics were washed with 5% aqueous LiCl solution, the washed organics were dried with Na₂SO₄, the dried solution was filtered, and the filtered solution was concentrated. The resulting crude was purified by silica gel chromatography (10-70% EtOAc/heptanes) to provide the title compound. MS (ESI⁻) m/z 473.4 (M−H).

### Example 1-C

### Methyl 2-(2-(3′-(aminomethyl)-[1,1′-biphenyl]-3-ylcarboxamido)phenyl)acetate

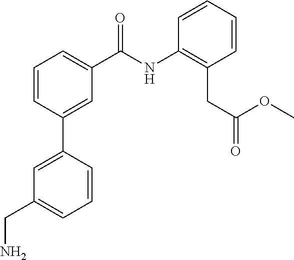 [SMILES: COC(=O)Cc1ccccc1NC(=O)c1cccc(-c2cccc(CN)c2)c1]

Methyl 2-(2-(3′-(((tert-butoxycarbonyl)amino)methyl)-[1,1′-biphenyl]-3-ylcarboxamido)phenyl)acetate (50 mg, 0.105 mmol) was added to a solution of HCl (2 M in Et₂O, 1.05 mL, 2.11 mmol). The resulting mixture was stirred at room temperature overnight, then concentrated. The residue was purified by reverse phase HPLC using condition A. Fractions containing the desired product were pooled, the pooled fractions were frozen and lyophilized to provide the title compound as a TFA salt. ¹H NMR (TFA salt, 400 MHz, methanol-d₄) δ ppm 8.26 (s, 1H), 7.98 (d, J=7.7 Hz, 1H), 7.88-7.94 (m, 1H), 7.77-7.84 (m, 2H), 7.65 (t, J=7.8 Hz, 1H), 7.59 (t, J=7.8 Hz, 1H), 7.47-7.55 (m, 2H), 7.34-7.42 (m, 2H), 7.25-7.32 (m, 1H), 4.22 (s, 2H), 3.78 (s, 2H), 3.63 (s, 3H). HRMS calcd. for C₂₃H₂₂N₂O₃ (M+H)⁺375.1700. found 375.1696.

### Example 2

### Example 2-A

### Methyl 2-(2-(3-bromobenzamido)phenyl)acetate

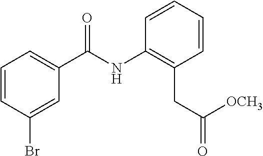 [SMILES: COC(=O)Cc1ccccc1NC(=O)c1cccc(Br)c1]

TEA (0.276 mL, 1.984 mmol) was added to a mixture of 3-bromobenzoic acid (199 mg, 0.992 mmol) and HATU (415 mg, 1.091 mmol) in DMF at 23° C. In a separate vial, methyl 2-(2-aminophenyl)acetate hydrochloride (CAS #49851-36-7) (200 mg, 0.992 mmol) was stirred with TEA (0.276 mL, 1.980 mmol) in DMF (0.5 mL). After 5 min, the solution of methyl 2-(2-aminophenyl)acetate hydrochloride (200 mg, 0.992 mmol) was added to the 3-bromobenzoic acid (199 mg, 0.992 mmol) mixture and the resulting mixture was stirred at rt for 30 min. The reaction mixture was diluted with EtOAc and washed with water. The aqueous layer was extracted with EtOAc. The combined organics were washed with 5% aq LiCl, dried (Na₂SO₄) and concentrated. The resulting residue was purified by silica gel chromatography (EtOAc-heptanes 0-60%) to provide the title compound. MS (ESI⁺) m/z 348.0, 350.0 (M+H).

### Example 2-B

### 2-(2-(3′-(Aminomethyl)-[1,1′-biphenyl]-3-ylcarboxamido)phenyl)acetic acid

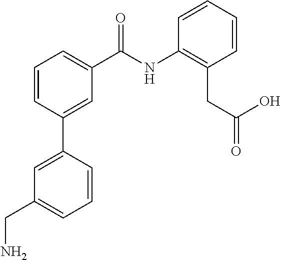 [SMILES: NCc1cccc(-c2cccc(C(=O)Nc3ccccc3CC(=O)O)c2)c1]

A mixture of methyl 2-(2-(3-bromobenzamido)phenyl)acetate (Example 2-A) (90 mg, 0.258 mmol), (3-(aminomethyl)phenyl)boronic acid hydrochloride (CAS #146285-80-5) (63.0 mg, 0.336 mmol), PdCl₂(dppf).CH₂Cl₂ adduct (CAS #95464-05-4) (10.55 mg, 0.013 mmol), and 2M aq. K₃PO₄ (0.388 mL, 0.775 mmol) in 9:1 MeCN/H₂O (2.8 mL) was heated in a microwave reactor at 110° C. for 60 min. The organic layer was filtered and the filtrate was directly purified by reverse phase HPLC (method B). Fractions containing the desired product were pooled, the pooled fractions were frozen and lyophilized to provide the title compound. ¹H NMR (400 MHz, DMSO-d₆, one drop of TFA added) δ ppm 10.13 (s, 1H) 8.28 (s, 1H) 8.23 (br s, 3H) 7.98 (d, J=7.83 Hz, 1H) 7.87-7.94 (m, 2H) 7.81 (d, J=7.83 Hz, 1H) 7.66 (t, J=7.77 Hz, 1H) 7.54-7.61 (m, 1H) 7.46-7.53 (m, 2H) 7.30-7.39 (m, 2H) 7.21-7.28 (m, 1H) 4.15 (q, J=5.81 Hz, 2H) 3.70 (s, 2H) HRMS calcd. for C₂₂H₂₀N₂O₃ (M+H)⁺ 361.1552. found 361.1550.

### Example 3

### Example 3-A

### N-(2-(2-Amino-2-oxoethyl)phenyl)-3-bromobenzamide

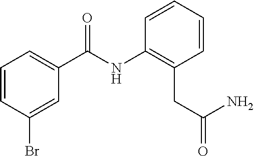 [SMILES: NC(=O)Cc1ccccc1NC(=O)c1cccc(Br)c1]

TEA (0.347 mL, 2.487 mmol) was added to a mixture of 3-bromobenzoic acid (250 mg, 1.244 mmol) and HATU (520 mg, 1.368 mmol) in DMF at 23° C. After 5 min, 2-(2-aminophenyl)acetamide (CAS #4103-60-0) (187 mg, 1.244 mmol) was added to the mixture and the resulting mixture was stirred at room temperature overnight. The reaction mixture was partitioned between EtOAc and H₂O. Layers were separated and the aqueous layer was extracted with 4:1 EtOAc/heptanes. The combined organics were washed with 5% aqueous LiCl solution, the combined organics were dried (Na₂SO₄), filtered, and concentrated. The residue was purified by silica gel chromatography (EtOAc-heptanes 10-70%) to provide the title compound. MS (ESI⁺) m/z 333.1, 335.1 (M+H).

### Example 3-B

### N-(2-(2-Amino-2-oxoethyl)phenyl)-3′-(aminomethyl)-[1,1′-biphenyl]-3-carboxamide

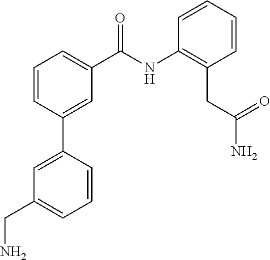 [SMILES: NCc1cccc(-c2cccc(C(=O)Nc3ccccc3CC(N)=O)c2)c1]

A mixture of N-(2-(2-amino-2-oxoethyl)phenyl)-3-bromobenzamide (50 mg, 0.150 mmol), 3-aminomethylphenylboronic acid hydrochloride (CAS #146285-80-5) (36.6 mg, 0.195 mmol), PdCl₂(dppf).CH₂Cl₂ adduct (CAS #95464-05-4) (6.13 mg, 7.50 μmol), and 2M aq. K₃PO₄ (0.225 mL, 0.450 mmol) in 9:1 MeCN/H₂O (1.5 mL) was heated in a microwave reactor at 110° C. for 60 min. The organic layer was filtered and the filtrate was purified directly with reverse phase HPLC (method B). Fractions containing the desired product were pooled, the pooled fractions were frozen and lyophilized to provide the title compound. ¹H NMR (400 MHz, CH₃OH+D₂O) ppm 8.35 (br. s., 1H) 8.02 (d, J=7.58 Hz, 1H) 7.86 (dd, J=13.33, 7.89 Hz, 2H) 7.74 (br. s., 1H) 7.54-7.68 (m, 2H) 7.45 (t, J=7.58 Hz, 1H) 7.28-7.41 (m, 3H) 7.14-7.26 (m, 1H) 3.91 (s, 2H) 3.64 (s, 2H). HRMS calcd. for C₂₂H₂₁N₃O₂ (M+H)⁺ 360.1712. found 360.1717.

### Example 4

### Example 4-A

### Methyl 2-(2-(5′-(aminomethyl)-2′-fluoro-[1,1′-biphenyl]-3-ylcarboxamido)phenyl)acetate

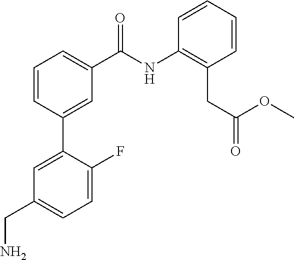 [SMILES: COC(=O)Cc1ccccc1NC(=O)c1cccc(-c2cc(CN)ccc2F)c1]

A mixture of methyl 2-(2-(3-bromobenzamido)phenyl)acetate (Example 2-A) (60 mg, 0.172 mmol), 5-(aminomethyl)-2-fluorophenylboronic acid hydrochloride (CAS #1072946-46-3) (46.0 mg, 0.224 mmol), PdCl₂(dppf).CH₂Cl₂ adduct (CAS #95464-05-4) (7.04 mg, 0.09 mmol), and 2M aq. K₃PO₄ (0.258 mL, 0.517 mmol) in 9:1 MeCN/H₂O (1.8 mL) was heated in a microwave reactor at 110° C. for 60 min. The organic layer was filtered and the filtrate was directly purified by reverse phase HPLC (CH₃CN-water (0.1% NH₄OH) 10-100%). Fractions containing the desired product were pooled, the pooled fractions were frozen and lyophilized to provide the title compound. ¹H NMR (400 MHz, METHANOL-d₄) δ ppm 8.16 (s, 1H) 8.02 (d, J=7.71 Hz, 1H) 7.82 (dd, J=7.77, 1.07 Hz, 1H) 7.61-7.73 (m, 2H) 7.46-7.58 (m, 2H) 7.24-7.42 (m, 4H) 4.19 (s, 2H) 3.77 (s, 2H) 3.63 (s, 3H). HRMS calcd. for C₂₃H₂₁FN₂O₃(M+H)⁺ 393.1614. found 393.1612.

### Example 4-B

### 2-(2-(5′-(Aminomethyl)-2′-fluoro-[1,1′-biphenyl]-3-ylcarboxamido)phenyl)acetic acid

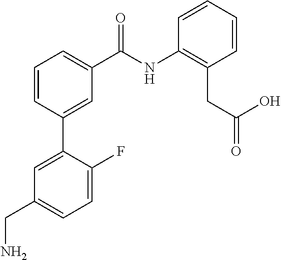 [SMILES: NCc1ccc(F)c(-c2cccc(C(=O)Nc3ccccc3CC(=O)O)c2)c1]

The title compound was obtained as a second product from the reaction in Example 4-A and purified in a similar fashion. ¹H NMR (400 MHz, METHANOL-d₄) δ ppm 8.18 (s, 1H) 8.03 (d, J=7.96 Hz, 1H) 7.82 (dd, J=7.77, 1.07 Hz, 1H) 7.58-7.74 (m, 3H) 7.51 (ddd, J=8.46, 4.55, 2.40 Hz, 1H) 7.22-7.42 (m, 4H) 4.19 (s, 2H) 3.73 (s, 2H). HRMS calcd. for C₂₂H₁₉FN₂O₃ (M+H)⁺ 379.1458. found 379.1463.

### Example 5

### 2-(2-(5-Acetamido-3′-(aminomethyl)-[1,1′-biphenyl]-3-ylcarboxamido)phenyl)acetic acid

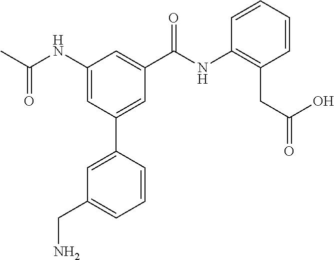 [SMILES: CC(=O)Nc1cc(C(=O)Nc2ccccc2CC(=O)O)cc(-c2cccc(CN)c2)c1]

The title compound as synthesized as described in Example 2 starting from 3-acetamido-5-bromobenzoic acid (CAS #78238-11-6). ¹H NMR (400 MHz, METHANOL-d₄) δ ppm 8.39 (t, J=1.77 Hz, 1H) 8.19 (t, J=1.52 Hz, 1H) 7.91-8.06 (m, 3H) 7.75 (d, J=7.96 Hz, 1H) 7.54 (t, J=7.64 Hz, 1H) 7.43 (d, J=7.71 Hz, 1H) 7.24-7.35 (m, 2H) 7.12 (td, J=7.52, 1.26 Hz, 1H) 4.19 (s, 2H) 3.62 (s, 2H) 2.19 (s, 3H). HRMS calcd. for C₂₄H₂₃FN₃O₄(M+H)⁺ 418.1767. found 418.1757.

### Example 6

### Example 6-A

### 2-(2-(3′-((tert-Butoxycarbonylamino)methyl)-5′-fluorobiphenyl-3-ylcarboxamido)phenyl)acetic acid

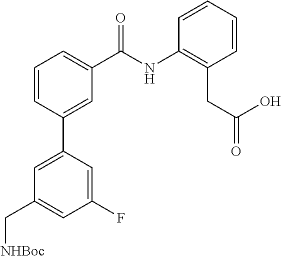 [SMILES: CC(C)(C)OC(=O)NCc1cc(F)cc(-c2cccc(C(=O)Nc3ccccc3CC(=O)O)c2)c1]

A mixture of methyl 2-(2-(3-bromobenzamido)phenyl)acetate (Example 2-A) (100 mg, 0.172 mmol), tert-butyl 3-fluoro-5-(4,4,5,5-tetramethyl-1,3,2-dioxaborolan-2-yl)benzylcarbamate (Intermediate 24) (101 mg, 0.316 mmol), PdCl₂(dppf).CH₂Cl₂ adduct (CAS #95464-05-4) (11.7 mg, 0.014 mmol), and 2M aq. K₃PO₄ (0.431 mL, 0.862 mmol) in MeCN (3 mL) was heated in a microwave reactor at 110° C. for 75 min. The reaction mixture was partitioned between EtOAc and water. The aqueous layer was extracted with EtOAc. The combined organics were washed with brine, dried (Na₂SO₄) and concentrated. The residue was purified by silica gel chromatography (EtOAc-heptane 0-100%) to provide the title compound. MS (ESI−) m/z 477.4 (M−H).

### Example 6-B

### 2-(2-(3′-(Aminomethyl)-5′-fluoro-[1,1′-biphenyl]-3-ylcarboxamido)phenyl)acetic acid

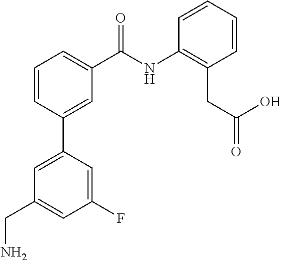 [SMILES: NCc1cc(F)cc(-c2cccc(C(=O)Nc3ccccc3CC(=O)O)c2)c1]

TFA (0.5 mL, 6.49 mmol) was added to a solution of 2-(2-(3′-((tert-butoxycarbonylamino)methyl)-5′-fluorobiphenyl-3-ylcarboxamido)phenyl)acetic acid (57 mg, 0.119 mmol) in DCM (2 mL) at rt. The reaction mixture was stirred at rt for 1 hr. The mixture was concentrated and the residue was purified by reverse phase HPLC (method B). Fractions containing the desired product were pooled, the pooled fractions were frozen and lyophilized to provide the title compound. ¹H NMR (400 MHz, METHANOL-d₄) δ ppm 8.46 (s, 1H) 8.08 (dt, J=7.80, 1.34 Hz, 1H) 7.85-7.97 (m, 2H) 7.81 (s, 1H) 7.65 (t, J=7.77 Hz, 1H) 7.56 (d, J=9.60 Hz, 1H) 7.27-7.38 (m, 2H) 7.20-7.27 (m, 1H) 7.17 (dd, J=7.45, 1.26 Hz, 1H) 4.23 (s, 2H) 3.65 (s, 2H). HRMS calcd. for C₂₂H₁₉FN₂O₃(M+H)⁺ 379.1458. found 379.1450.

### Example 7

### Example 7-A

### Methyl 2-(2-(3-bromo-5-(methylcarbamoyl)benzamido)phenyl)acetate

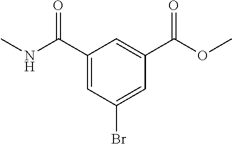

Sodium hydroxide (1N aqueous solution, 4.39 mL, 4.39 mmol) was added to a solution of dimethyl 5-bromoisophthalate (CAS #51760-21-5) (1.2 g, 4.39 mmol) in MeOH (21.97 mL)/THF (21.97 mL). The reaction mixture was stirred at room temperature overnight. It was then quenched with 1N HCl, extracted with EtOAc, dried and concentrated. The crude was dissolved in dichloromethane (46.3 mL), oxalyl chloride (0.487 mL, 5.56 mmol) and DMF (0.036 mL, 0.463 mmol) were added at 0° C. After 15 minutes, methanamine (23.16 mL, 46.3 mmol) was added and the reaction stirred for 2 h. The reaction mixture was purified using silica gel chromatography (EtOAc-heptane 1:1) to provide the title compound. MS (ESI⁺) m/z 272.0, 274.0 (M+H).

### Example 7-B

### 3-Bromo-5-(methylcarbamoyl)benzoic acid

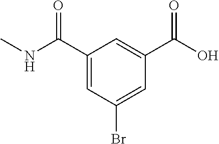

Methyl 2-(2-(3-bromo-5-(methylcarbamoyl)benzamido)phenyl)acetate (1.2 g, 4.41 mmol) was dissolved in a mixture of THF (26.5 mL), MeOH (8.82 mL), and H₂O (8.82 mL). LiOH (0.158 g, 6.62 mmol) was added and the reaction mixture was stirred at room temperature for 3 h. 1N HCl was added to quench the excess base and the resulting mixture was extracted with EtOAc. The organic layer was separated, dried (Na₂SO₄), filtered and concentrated. The resulting residue was used in the next step without further purification. MS (ESI−) m/z 256.3, 258.3 (M−H).

### Example 7-C

### Methyl 2-(2-(3-bromo-5-(methylcarbamoyl)benzamido)phenyl)acetate

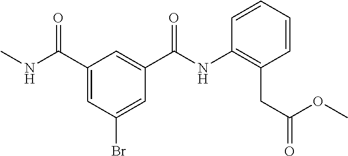

To a solution of 3-bromo-5-(methylcarbamoyl)benzoic acid (210 mg, 0.814 mmol) in dichloromethane (8 mL), oxalyl chloride (0.107 mL, 1.22 mmol) and DMF (0.006 mL, 0.081 mmol) were added at 0° C. After 15 minutes, methyl 2-(2-aminophenyl)acetate hydrochloride (CAS #35613-44-6) (269 mg, 1.63 mmol) was added and the reaction stirred for 2 h. The reaction mixture was purified using silica gel chromatography (EtOAc-heptane 1:1) to provide the title compound. MS (ESI⁺) m/z 405.2, 407.1 (M+H).

### Example 7-D

### 2-(2-(3′-(Aminomethyl)-5-(methylcarbamoyl)-[1,1′-biphenyl]-3-ylcarboxamido)phenyl)acetic acid

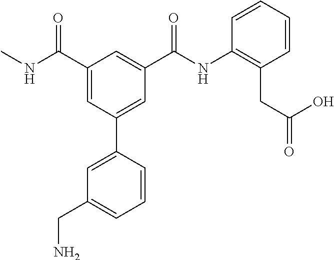 [SMILES: CNC(=O)c1cc(C(=O)Nc2ccccc2CC(=O)O)cc(-c2cccc(CN)c2)c1]

A mixture of methyl 2-(2-(3-bromo-5-(methylcarbamoyl)benzamido)phenyl)acetate (60 mg, 0.148 mmol), 3-(aminomethyl)phenylboronic acid hydrochloride (36.1 mg, 0.192 mmol), 2 M K₃PO₄ (0.222 mL, 0.444 mmol), and PdCl₂(dppf).CH₂Cl₂ adduct (CAS #95464-05-4) (6.05 mg, 7.40 μmol) in 9:1 MeCN/H₂O (1.5 mL) was heated in a microwave reactor at 110° C. for 60 min. The organic layer was filtered and the filtrate was concentrated. The residue was purified with reverse phase HPLC (method A) to give the title compound as TFA salt. ¹H NMR (TFA salt, 400 MHz, METHANOL-d₄) δ ppm 8.42 (dd, J=3.28, 1.52 Hz, 2H) 8.33 (t, J=1.64 Hz, 1H) 7.80-7.92 (m, 2H) 7.57-7.67 (m, 2H) 7.53 (s, 1H) 7.33-7.45 (m, 2H) 7.29 (dd, J=7.33, 1.26 Hz, 1H) 4.23 (s, 2H) 3.76 (s, 2H) 2.98 (s, 3H). HRMS calcd. for C₂₄H₂₃N₃O₄ (M+H)⁺ 418.1767. found 418.1755.

### Example 8

### Example 8-A

### Methyl 2-(2-(3′-(aminomethyl)-6-methoxy-[1,1′-biphenyl]-3-ylcarboxamido)phenyl)acetate

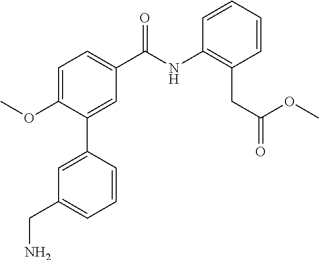

The title compound as synthesized as described in Example 2 starting from 3-bromo-4-methoxybenzoic acid (CAS #99-58-1). ¹H NMR (400 MHz, METHANOL-d₄) δ ppm 7.91-8.03 (m, 2H) 7.44-7.58 (m, 3H) 7.17-7.44 (m, 6H) 3.84-3.94 (m, 5H) 3.75 (s, 2H) 3.61 (s, 3H). HRMS calcd. for C₂₄H₂₄N₂O₄ (M+H)⁺ 405.1814. found 405.1809.

### Example 8-B

### 2-(2-(3′-(Aminomethyl)-6-methoxy-[1,1′-biphenyl]-3-ylcarboxamido)phenyl)acetic acid

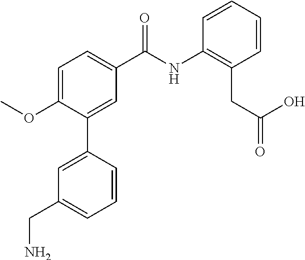 [SMILES: COc1ccc(C(=O)Nc2ccccc2CC(=O)O)cc1-c1cccc(CN)c1]

The title compound was obtained as a second product from the reaction in Example 8-A and purified in a similar fashion. ¹H NMR (400 MHz, METHANOL-d₄) δ ppm 8.21 (d, J=2.40 Hz, 1H) 8.09 (dd, J=8.72, 2.40 Hz, 1H) 7.91 (d, J=7.07 Hz, 1H) 7.72-7.82 (m, 2H) 7.41-7.49 (m, 1H) 7.34-7.41 (m, 1H) 7.21-7.33 (m, 3H) 7.07-7.14 (m, 1H) 4.17 (s, 2H) 3.92 (s, 3H) 3.58 (s, 2H). HRMS calcd. for C₂₃H₂₂N₂O₄ (M+H)⁺ 391.1658. found 391.1653.

### Example 9

### Methyl 2-(2-(3′-(aminomethyl)-5′-fluoro-6-methoxy-[1,1′-biphenyl]-3-ylcarboxamido)phenyl)acetate

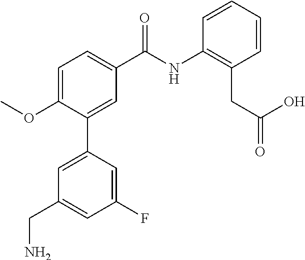 [SMILES: COc1ccc(C(=O)Nc2ccccc2CC(=O)O)cc1-c1cc(F)cc(CN)c1]

The title compound as synthesized as described in Example 8 using tert-butyl 3-fluoro-5-(4,4,5,5-tetramethyl-1,3,2-dioxaborolan-2-yl)benzylcarbamate (Intermediate 24). ¹H NMR (400 MHz, METHANOL-d₄) δ ppm 8.05 (dd, J=8.59, 2.40 Hz, 1H) 8.00 (d, J=2.40 Hz, 1H) 7.59 (d, J=7.45 Hz, 1H) 7.48 (s, 1H) 7.39-7.45 (m, 1H) 7.31-7.39 (m, 2H) 7.17-7.29 (m, 3H) 4.19 (s, 2H) 3.93 (s, 3H) 3.71 (s, 2H). HRMS calcd. for C₂₃H₂₁FN₂O₄(M+H)⁺ 409.1564. found 409.1556.

### Example 10

### Example 10-A

### 2-(2-(3′-(N-(tert-Butoxycarbonyl)carbamimidoyl)biphenyl-3-ylcarboxamido)phenyl)acetic acid

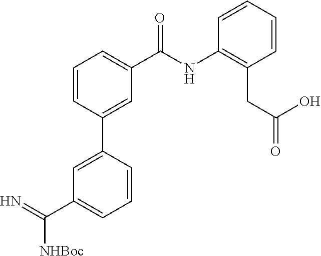 [SMILES: CC(C)(C)OC(=O)NC(=N)c1cccc(-c2cccc(C(=O)Nc3ccccc3CC(=O)O)c2)c1]

A degassed mixture of methyl 2-(2-(3-bromobenzamido)phenyl)acetate (Example 2-A) (50 mg, 0.144 mmol), tert-butyl imino(3-(4,4,5,5-tetramethyl-1,3,2-dioxaborolan-2-yl)phenyl)methylcarbamate (Intermediate 28-B) (80 mg, 0.230 mmol), PdCl₂(dppf).CH₂Cl₂ adduct (CAS #95464-05-4) (5.86 mg, 7.18 μmol), and 2M aq. K₃PO₄ (0.215 ml, 0.431 mmol) in CH₃CN (1.5 mL) was heated in a microwave at 110° C. for 1 hr. The reaction mixture was diluted with EtOAc and washed with water. The aqueous layer was washed with EtOAc. The combined organics were washed with brine, dried (Na₂SO₄) and concentrated. The residue was purified by silica gel chromatography (DCM-MeOH 0-20%) to provide the title compound. MS (ESI⁺) m/z 474.4 (M+H).

### Example 10-B

### 2-(2-(3′-Carbamimidoyl-[1,1′-biphenyl]-3-ylcarboxamido)phenyl)acetic acid

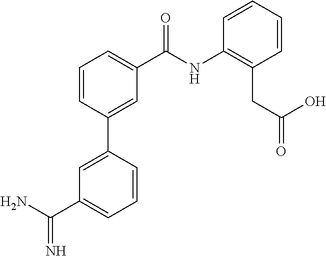 [SMILES: N=C(N)c1cccc(-c2cccc(C(=O)Nc3ccccc3CC(=O)O)c2)c1]

TFA (0.5 ml, 0.034 mmol) was added to a solution of 2-(2-(3′-(N-(tert-butoxycarbonyl)carbamimidoyl)biphenyl-3-ylcarboxamido)phenyl)acetic acid (16 mg, 0.034 mmol) in DCM (2 ml) and the reaction mixture was stirred at rt for 2 hr. The mixture was concentrated and the residue was purified by reverse phase HPLC (method B). Fractions containing the desired product were pooled, the pooled fractions were frozen and lyophilized to provide the title compound. ¹H NMR (400 MHz, DMSO-d₆) δ ppm 13.15 (s, 1H) 9.30 (br. s., 3H) 8.84 (s, 1H) 8.57 (s, 1H) 7.98-8.18 (m, 4H) 7.84 (d, J=8.08 Hz, 1H) 7.67 (t, J=7.77 Hz, 2H) 7.16-7.30 (m, 2H) 6.99-7.11 (m, 1H) 3.49 (s, 2H). HRMS calcd. for C₂₂H₁₉N₃O₃ (M+H)⁺ 374.1498. found 374.1505.

### Example 11

### Example 11-A

### tert-Butyl 2-(2-(3,5-dibromobenzamido)phenyl)acetate

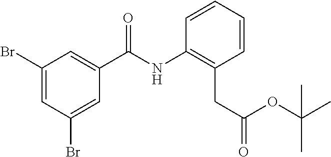 [SMILES: CC(C)(C)OC(=O)Cc1ccccc1NC(=O)c1cc(Br)cc(Br)c1]

TEA (6.72 mL, 48.2 mmol) was added to a mixture of 3,5-dibromobenzoic acid (CAS #618-58-6) (7 g, 25.01 mmol), tert-butyl 2-(2-aminophenyl)acetate (CAS #98911-34-3) (5 g, 24.12 mmol) and HATU (9.63 g, 25.3 mmol) in DMF at 23° C. The brown mixture was stirred at rt overnight. The mixture was partitioned between 1:1 EtOAc/heptane and water. The aqueous layer was extracted with 1:1 EtOAc/heptane. The combined organic layers were washed with brine, dried (Na₂SO₄) and concentrated to provide the title compound, which was used in the next reaction without further purification. MS (ESI−) m/z 468.2, 466.2, 470.2 (M−H).

### Example 11-B

### tert-Butyl 2-(2-(5-bromo-3′-(((tert-butoxycarbonyl)amino)methyl)-5′-fluoro-[1,1′-biphenyl]-3-ylcarboxamido)phenyl)acetate

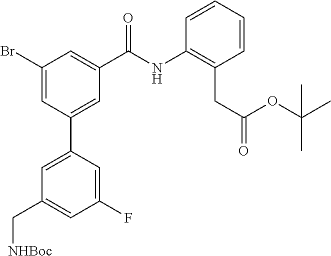 [SMILES: CC(C)(C)OC(=O)Cc1ccccc1NC(=O)c1cc(Br)cc(-c2cc(F)cc(CNC(=O)OC(C)(C)C)c2)c1]

A degassed mixture of tert-butyl 2-(2-(3,5-dibromobenzamido)phenyl)acetate (11.32 g, 24.13 mmol), tert-butyl 3-fluoro-5-(4,4,5,5-tetramethyl-1,3,2-dioxaborolan-2-yl)benzylcarbamate (Intermediate 24) (5.30 g, 12.06 mmol), PdCl₂(dppf).CH₂Cl₂ adduct (CAS #95464-05-4) (0.985 g, 1.206 mmol), and 2M aq. K₃PO₄ (24.13 mL, 48.3 mmol) in CH₃CN (30 mL) was heated at 80° C. for 2 hr. The reaction mixture was diluted with EtOAc and washed with water. The aqueous layer was extracted with EtOAc. The combined organics were washed with brine, dried (Na₂SO₄) and concentrated. The residue was purified by FCC (EtOAc-heptane 0-50%) to provide the title compound. MS (ESI−) m/z 611.5, 613.5 (M−H).

### Example 11-C

### tert-Butyl 2-(2-(3′-(((tert-butoxycarbonyl)amino)methyl)-5′-fluoro-5-(isoxazol-4-yl)-[1,1′-biphenyl]-3-ylcarboxamido)phenyl)acetate

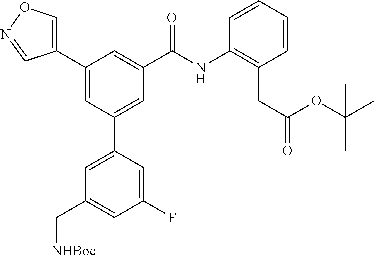 [SMILES: CC(C)(C)OC(=O)Cc1ccccc1NC(=O)c1cc(-c2cnoc2)cc(-c2cc(F)cc(CNC(=O)OC(C)(C)C)c2)c1]

A degassed mixture of tert-butyl 2-(2-(5-bromo-3′-(((tert-butoxycarbonyl)amino)methyl)-5′-fluoro-[1,1′-biphenyl]-3-ylcarboxamido)phenyl)acetate (60 mg, 0.098 mmol), isoxazol-4-ylboronic acid (CAS #1008139-25-0) (33.1 mg, 0.293 mmol), X-Phos palladacycle (CAS 1028206-56-5) (3.61 mg, 4.89 μmol), and 2M aq. K₃PO₄ (0.147 mL, 0.293 mmol) in DMF (1 mL) was heated at 90° C. for 2 hr. The reaction mixture was diluted with 1:1 EtOAc-heptane and washed with water. The aqueous layer was extracted with 1:1 EtOAc-heptane. The combined organics were washed with brine, dried (Na₂SO₄) and concentrated to provide crude title compound which was used as is in the next step. MS (ESI⁻) m/z 600.7 (M−H).

### Example 11-D

### 2-(2-(3′-(Aminomethyl)-5′-fluoro-5-(isoxazol-4-yl)-[1,1′-biphenyl]-3-ylcarboxamido)phenyl)acetic acid

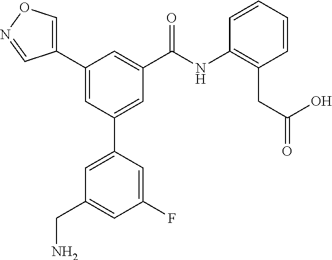 [SMILES: NCc1cc(F)cc(-c2cc(C(=O)Nc3ccccc3CC(=O)O)cc(-c3cnoc3)c2)c1]

TFA (300 μl, 3.89 mmol) was added to a solution of crude tert-butyl 2-(2-(3′-(((tert-butoxycarbonyl)amino)methyl)-5′-fluoro-5-(isoxazol-4-yl)-[1,1′-biphenyl]-3-ylcarboxamido)phenyl)acetate (30.1 mg, 0.049 mmol) in DCM (2 mL) at rt. The reaction mixture was stirred at rt overnight, then concentrated. The residue was purified by reverse phase HPLC (method B). Fractions containing the desired product were pooled, the pooled fractions were frozen and lyophilized to provide the title compound. ¹H NMR (400 MHz, DMSO-d₆) δ ppm 8.72 (s, 1H) 8.51 (s, 1H) 8.16 (s, 1H) 7.70-7.89 (m, 2H) 7.61 (d, J=7.58 Hz, 1H) 7.50 (d, J=9.73 Hz, 1H) 7.08-7.38 (m, 5H) 4.15 (s, 2H) 3.62 (s, 2H). HRMS calcd. for C₂₅H₂₀FN₃O₄(M+H)⁺ 446.1516. found 446.1497.

### Example 12

### 2-(2-(3′-(Aminomethyl)-5-bromo-5′-fluoro-[1,1′-biphenyl]-3-ylcarboxamido)phenyl)acetic acid

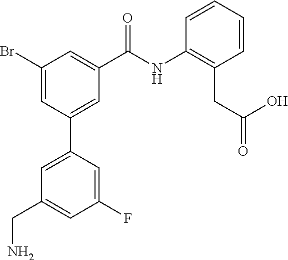 [SMILES: NCc1cc(F)cc(-c2cc(Br)cc(C(=O)Nc3ccccc3CC(=O)O)c2)c1]

The title compound was obtained as a side product from the same reaction Example 11-C and was isolated using the same HPLC condition. ¹H NMR (400 MHz, DMSO-d₆) δ ppm 13.10 (s, 1H) 8.70 (s, 1H) 8.23 (t, J=1.58 Hz, 1H) 8.05-8.14 (m, 2H) 7.98 (d, J=7.20 Hz, 1H) 7.78 (d, J=9.98 Hz, 1H) 7.32 (d, J=9.47 Hz, 1H) 7.19-7.28 (m, 2H) 7.06 (td, J=7.45, 1.26 Hz, 1H) 4.13 (s, 2H) 3.54 (s, 2H). HRMS calcd. for C₂₂H₁₈BrFN₂O₃(M+H)⁺ 457.0563 and 459.0543 found 457.0539 and 459.0522.

### Example 13

### Example 13-A

### tert-Butyl 2-(2-(3′-(((tert-butoxycarbonyl)amino)methyl)-5′-fluoro-5-(phenylamino)-[1,1′-biphenyl]-3-ylcarboxamido)phenyl)acetate

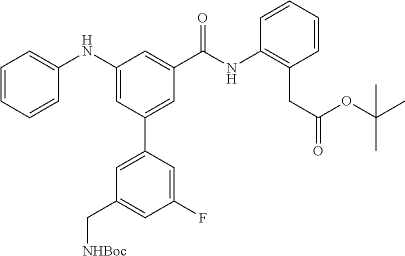 [SMILES: CC(C)(C)OC(=O)Cc1ccccc1NC(=O)c1cc(Nc2ccccc2)cc(-c2cc(F)cc(CNC(=O)OC(C)(C)C)c2)c1]

A degassed mixture of tert-butyl 2-(2-(5-bromo-3′-(((tert-butoxycarbonyl)amino)methyl)-5′-fluoro-[1,1′-biphenyl]-3-ylcarboxamido)phenyl)acetate (Example 11-B) (60 mg, 0.098 mmol), aniline (0.027 mL, 0.293 mmol), X-Phos palladacycle (CAS 1028206-56-5) (3.61 mg, 4.89 μmol), and 2M aq. K₃PO₄ (104 mg, 0.489 mmol) in CH₃CN (1 mL) was heated at 90° C. for 2 hr. The reaction mixture was diluted with 1:1 EtOAc-heptane and washed with water. The aqueous layer was extracted with 1:1 EtOAc-heptane. The combined organics were washed with brine, dried (Na₂SO₄) and concentrated to provide the title compound crude, which was used in the next reaction without further purification. MS (ESI⁻) m/z 624.7 (M−H).

### Example 13-B

### 2-(2-(3′-(Aminomethyl)-5′-fluoro-5-(phenylamino)-[1,1′-biphenyl]-3-ylcarboxamido)phenyl)acetic acid

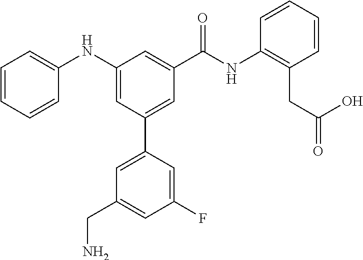 [SMILES: NCc1cc(F)cc(-c2cc(Nc3ccccc3)cc(C(=O)Nc3ccccc3CC(=O)O)c2)c1]

The title compound was obtained from tert-butyl 2-(2-(3′-(((tert-butoxycarbonyl)amino)methyl)-5′-fluoro-5-(phenylamino)-[1,1′-biphenyl]-3-ylcarboxamido)phenyl)acetate as reported in Example 11-D. ¹H NMR (400 MHz, DMSO-d₆) δ ppm 12.56 (br. s., 1H) 8.60-9.12 (m, 2H) 8.51 (s, 1H) 8.10 (d, J=7.58 Hz, 2H) 7.97 (d, J=7.58 Hz, 1H) 7.67 (s, 1H) 7.50-7.60 (m, 2H) 7.15-7.37 (m, 7H) 7.04 (td, J=7.45, 1.26 Hz, 1H) 6.92 (t, J=7.26 Hz, 1H) 4.14 (s, 2H) 3.52 (s, 2H). HRMS calcd. for C₂₈H₂₄FN₃O₃(M+H)⁺ 470.1880. found 470.1870.

### Example 14

The compounds in the table below were synthesized as described in Example 13-B.

| | | Coupling partner in | | |
|---|---|---|---|---|
| | Structure/Chemical Name | place of aniline | ¹H NMR | HRMS |
| 14-A | 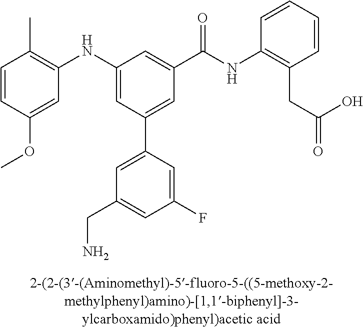 [SMILES: COc1ccc(C)c(Nc2cc(C(=O)Nc3ccccc3CC(=O)O)cc(-c3cc(F)cc(CN)c3)c2)c1] | 5-methoxy-2-methylaniline (CAS #50868-72-9) | (400 MHz, DMSO-d₆) δ ppm 12.50 (s, 1 H) 8.07 (br. s., 2 H) 7.93-7.98 (m, 1 H) 7.81 (s, 1 H), 7.39- 7.51 (m, 3 H) 7.27 (d, J = 9.22 Hz, 1 H) 7.18-7.23 (m, 2 H) 7.16 (d, J = 8.84 Hz, 1 H) 7.05 (dd, J = 7.45, 1.26 Hz, 1 H) 6.80 (d, J = 2.65 Hz, 1 H) 6.59 (dd, J = 8.34, 2.65 Hz, 1 H) 4.12 (s, 2 H) 3.69 (s, 3 H) 3.51 (s, 2 H) 2.16 (s, 3 H). | calcd. for C₃₀H₂₈FN₃O₄ (M + H)⁺ 514.2142, found 514.2111. |
| 14-B | 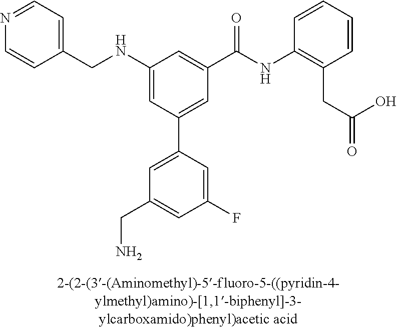 [SMILES: NCc1cc(F)cc(-c2cc(NCc3ccncc3)cc(C(=O)Nc3ccccc3CC(=O)O)c2)c1] | pyridin-3-ylmethanamine (CAS #3731-53-1) | (400 MHz, DMSO-d₆) δ ppm 12.45 (br. s., 1 H) 8.46-8.54 (m, 2 H) 8.02 (s, 1 H) 7.94 (d, J = 7.96 Hz, 1 H) 7.86 (s, 1 H) 7.46 (d, J = 9.85 Hz, 1 H) 7.40 (d, J = 5.94 Hz, 2 H) 7.14-7.27 (m, 4 H) 7.10 (s, 1 H) 7.02 (td, J = 7.45, 1.26 Hz, 1 H) 6.78 (t, J = 6.19 Hz, 1 H) 4.49 (d, J = 6.06 Hz, 2 H) 4.08 (s, 2 H) 3.47 (s, 2 H). | calcd. for C₂₈H₂₅FN₄O₃ (M + H)⁺ 485.1989, found 485.1966. |
| 14-C | 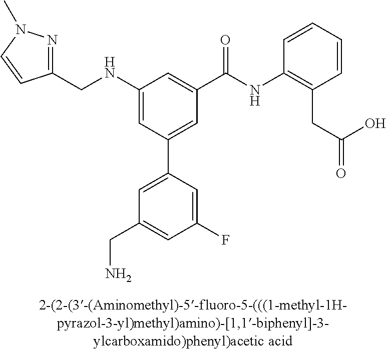 [SMILES: Cn1ccc(CNc2cc(C(=O)Nc3ccccc3CC(=O)O)cc(-c3cc(F)cc(CN)c3)c2)n1] | (1-methyl-1H-pyrzol-3-yl) methanamine (CAS #612511-81-6) | NMR (400 MHz, DMSO-d₆) δ ppm 12.26 (s, 1 H) 8.06 (s, 1 H) 7.95 (d, J = 7.20 Hz, 1 H) 7.85 (s, 1 H) 7.58 (d, J = 2.15 Hz, 1 H) 7.50 (d, J = 9.85 Hz, 1 H) 7.16-7.30 (m, 5 H) 7.03 (td, J = 7.42, 1.20 Hz, 1 H) 6.34-6.42 (m, 1 H) 6.18 (d, J = 2.15 Hz, 1 H) 4.31 (d, J = 5.81 Hz, 2 H) 4.11 (s, 2 H) 3.80 (s, 3 H) 3.49 (s, 2 H). | calcd. for C₂₇H₂₆FN₅O₃ (M + H)⁺ 488.2093, found 488.2074. |
| 14-D | 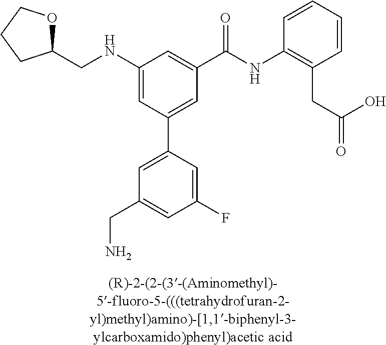 [SMILES: NCc1cc(F)cc(-c2cc(NC[C@H]3CCCO3)cc(C(=O)Nc3ccccc3CC(=O)O)c2)c1] | (R)-(tetrahydrofuran-2- yl)methanamine (CAS #7202-43-9) | (400 MHz, DMSO-d₆) δ ppm 12.27 (s, 1 H) 8.06 (s, 1 H) 7.93-8.00 (m, 1 H) 7.84 (s, 1 H) 7.56 (d, J = 9.98 Hz, 1 H) 7.14-7.29 (m, 5 H) 7.03 (td, J = 7.42, 1.20 Hz, 1 H) 6.01 (t, J = 5.81 Hz, 1 H) 4.11 (s, 2 H) 3.99-4.09 (m, 1 H) 3.76-3.85 (m, 1 H) 3.62- 3.72 (m, 1 H) 3.50 (s, 2 H) 3.16-3.29 (m, 2 H) 1.94- 2.06 (m, 1 H) 1.76-1.94 (m, 2 H) 1.57-1.69 (m, 1 H). | calcd. for C₂₇H₂₈FN₃O₄ (M + H)⁺ 478.2142, found 478.2125. |
| 14-E | 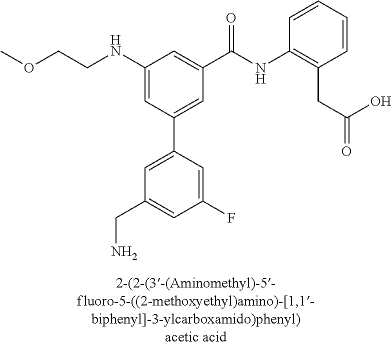 [SMILES: COCCNc1cc(C(=O)Nc2ccccc2CC(=O)O)cc(-c2cc(F)cc(CN)c2)c1] | 2-methoxyethanamine (CAS #109-85-3) | (400 MHz, DMSO-d₆) δ ppm 12.27 (br. s., 1 H) 8.93 (br. s., 2 H) 8.08 (s, 1 H) 7.96 (d, J = 7.33 Hz, 1 H) 7.85 (s, 1 H) 7.56 (d, J = 10.11 Hz, 1 H) 7.12- 7.28 (m, 5 H) 7.03 (td, J = 7.39, 1.14 Hz, 1 H) 6.01 (t, J = 5.68 Hz, 1 H) 4.12 (s, 2 H) 3.52-3.59 (m, 2 H) 3.50 (s, 2 H) 3.32-3.36 (m, 2 H) 3.31 (s, 3 H). | calcd. for C₂₅H₂₆FN₃O₄ (M + H)⁺ 452.1986, found 452.1971. |

### Example 15

### Example 15-A

### (S)-tert-Butyl 2-(2-(3′-(((tert-butoxycarbonyl)amino)methyl)-5′-fluoro-5-((tetrahydrofuran-2-yl)methoxy)-[1,1′-biphenyl]-3-ylcarboxamido)phenyl)acetate

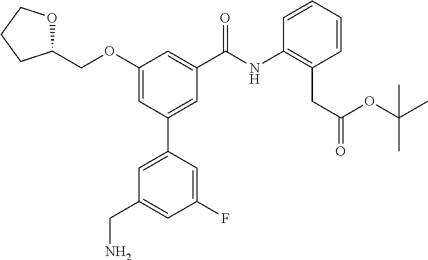 [SMILES: CC(C)(C)OC(=O)Cc1ccccc1NC(=O)c1cc(OC[C@@H]2CCCO2)cc(-c2cc(F)cc(CN)c2)c1]

A degassed mixture of tert-butyl 2-(2-(5-bromo-3′-(((tert-butoxycarbonyl)amino)methyl)-5′-fluoro-[1,1′-biphenyl]-3-ylcarboxamido)phenyl)acetate (Example 11-B) (100 mg, 0.163 mmol), (S)-(tetrahydrofuran-2-yl)methanol (CAS #57203-01-7) (41.6 mg, 0.407 mmol), Rockphos (CAS #1262046-34-3) (18.05 mg, 0.024 mmol), allylchloropalladium dimer (CAS #12012-95-2) (2.98 mg, 8.15 μmol), and Cs₂CO₃ (106 mg, 0.326 mmol) in toluene (2 mL) was heated at 90° C. overnight. The reaction mixture was diluted with EtOAc and washed with water. The aqueous layer was extracted with EtOAc. The combined organics were washed with brine, dried (Na₂SO₄) and concentrated to provide the crude title compound which was used in the next reaction without further purification. MS (ESI−) m/z 633.7 (M−H).

### Example 15-B

### (S)-2-(2-(3′-(Aminomethyl)-5′-fluoro-5-((tetrahydrofuran-2-yl)methoxy)-[1,1′-biphenyl]-3-ylcarboxamido)phenyl)acetic acid

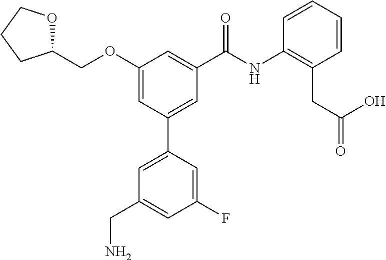 [SMILES: NCc1cc(F)cc(-c2cc(OC[C@@H]3CCCO3)cc(C(=O)Nc3ccccc3CC(=O)O)c2)c1]

TFA (500 μL, 6.49 mmol) was added to a solution of crude (S)-tert-butyl 2-(2-(3′-(((tert-butoxycarbonyl)amino)methyl)-5′-fluoro-5-((tetrahydrofuran-2-yl)methoxy)-[1,1′-biphenyl]-3-ylcarboxamido)phenyl)acetate (103 mg, 0.163 mmol) in DCM (3 mL) and the resulting mixture was stirred at rt overnight. The reaction mixture was concentrated and the residue was purified by reverse phase HPLC (method B). Fractions containing the desired product were pooled, the pooled fractions were frozen and lyophilized to provide the title compound. ¹H NMR (400 MHz, DMSO-d₆) δ ppm 12.71 (s, 1H) 8.30 (s, 1H) 8.11 (s, 1H) 7.98 (d, J=7.96 Hz, 1H) 7.74 (d, J=10.10 Hz, 1H) 7.57 (d, J=1.26 Hz, 1H) 7.50 (d, J=2.15 Hz, 1H) 7.17-7.31 (m, 3H) 7.05 (td, J=7.45, 1.26 Hz, 1H) 4.18-4.27 (m, 1H) 4.03-4.18 (m, 4H) 3.76-3.87 (m, 1H) 3.67-3.76 (m, 1H) 3.52 (s, 2H) 1.98-2.10 (m, 1H) 1.79-1.98 (m, 2H) 1.68-1.79 (m, 1H). HRMS calcd. for C₂₇H₂₇FN₂O₅(M+H)⁺ 479.1971. found 479.1982.

### Example 16

### Example 16-A

### tert-Butyl ((3′-ethynyl-[1,1′-biphenyl]-3-yl)methyl)carbamate

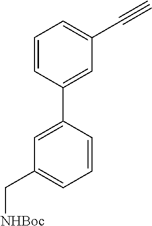 [SMILES: C#Cc1cccc(-c2cccc(CNC(=O)OC(C)(C)C)c2)c1]

A mixture of ((3-bromophenyl)ethynyl)trimethylsilane (CAS #3983-13-7) (500 mg, 1.975 mmol), (3-(((tert-butoxycarbonyl)amino)methyl)phenyl)boronic acid (CAS #832114-05-3) (545 mg, 2.172 mmol), PdCl₂(dppf).CH₂Cl₂ adduct (CAS #95464-05-4) (81 mg, 0.099 mmol) and 2M aq. K₃PO₄ (1.975 mL, 3.95 mmol) in CH₃CN (10 mL) was heated at 110° C. in the microwave for 1 hr. The reaction mixture was partitioned between EtOAc and sat. NH₄Cl. The aqueous layer was extracted with EtOAc. The combined organics were washed with brine, dried and concentrated. The crude mixture was dissolved in MeOH (5 mL) and K₂CO₃ (84 mg, 0.606 mmol) was added. The resulting mixture was stirred at rt overnight then concentrated. The residue was purified by FCC (EtOAc-heptane 0-40%) to provide the title compound. ¹H NMR (400 MHz, DICHLOROMETHANE-d₂) δ ppm 7.77 (t, J=1.64 Hz, 1H) 7.64 (dt, J=7.71, 1.52 Hz, 1H) 7.50-7.57 (m, 3H) 7.41-7.49 (m, 2H) 7.33 (d, J=7.45 Hz, 1H) 4.39 (br. s., 2H) 3.21 (s, 1H) 1.49 (s, 9H).

### Example 16-B

### Methyl 2-(2-((3′-(((tert-butoxycarbonyl)amino)methyl)-[1,1′-biphenyl]-3-yl)ethynyl)phenyl)acetate

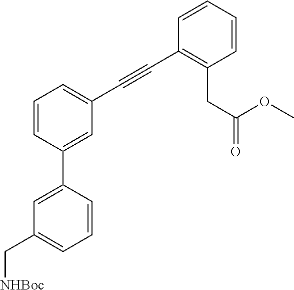 [SMILES: COC(=O)Cc1ccccc1C#Cc1cccc(-c2cccc(CNC(=O)OC(C)(C)C)c2)c1]

A mixture of tert-butyl ((3′-ethynyl-[1,1′-biphenyl]-3-yl)methyl)carbamate (124 mg, 0.403 mmol), methyl 2-(2-iodophenyl)acetate (CAS #66370-75-0) (557 mg, 2.017 mmol), Cul (15.37 mg, 0.081 mmol), Pd(PPh₃)₄ (46.6 mg, 0.040 mmol) in toluene (3 mL) at −78° C. was left on high vacuum for 3 min, then back-flushed with N₂. Diisopropylamine (0.172 mL, 1.210 mmol) was added and the resulting mixture was warmed to rt and stirred overnight. The reaction mixture was partitioned between EtOAc and water. The aqueous layer was extracted with EtOAc. The combined organics were washed with brine, dried (Na₂SO₄) and concentrated. The residue was purified by FCC (EtOAc-heptane 0-40%) to provide the title compound. ¹H NMR (400 MHz, DICHLOROMETHANE-d₂) δ ppm 7.82 (t, J=1.77 Hz, 1H) 7.60-7.66 (m, 2H) 7.54-7.60 (m, 3H) 7.45-7.52 (m, 2H) 7.32-7.40 (m, 4H) 4.41 (d, J=5.94 Hz, 2H) 3.97 (s, 2H) 3.72 (s, 3H) 1.49 (s, 9H).

### Example 16-C

### 2-(2-((3′-(Aminomethyl)-[1,1′-biphenyl]-3-yl)ethynyl)phenyl)acetic acid

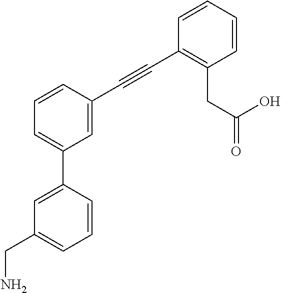 [SMILES: NCc1cccc(-c2cccc(C#Cc3ccccc3CC(=O)O)c2)c1]

TFA (1 mL, 12.98 mmol) was added to a solution of methyl 2-(2-((3′-(((tert-butoxycarbonyl)amino)methyl)-[1,1′-biphenyl]-3-yl)ethynyl)phenyl)acetate (75 mg, 0.165 mmol) in DCM (5 mL) at room temperature. After 2 hr, the mixture was concentrated and the residue was dissolved in acetonitrile (2 mL). LiOH (2M aq., 1 mL, 1.00 mmol) was added and the reaction mixture was stirred at rt overnight. The mixture was filtered and loaded directly onto reverse phase HPLC (method B). Fractions containing the desired product were pooled, the pooled fractions were frozen and lyophilized to provide the title compound. ¹H NMR (400 MHz, METHANOL-d₄) δ ppm 7.88 (t, J=1.45 Hz, 1H) 7.69 (s, 1H) 7.16-7.66 (m, 10H) 3.91 (s, 2H) 3.81 (s, 2H). HRMS calcd. for C₂₃H₁₉NO₂ (M+H)⁺ 342.1494. found 342.1495.

### Example 16-D

### 2-(2-(2-(3′-(Aminomethyl)-[1,1′-biphenyl]-3-yl)ethyl)phenyl)acetic acid

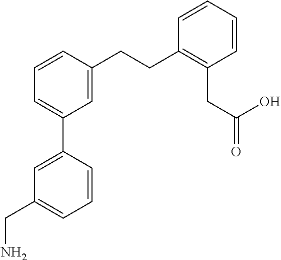 [SMILES: NCc1cccc(-c2cccc(CCc3ccccc3CC(=O)O)c2)c1]

A solution of methyl 2-(2-((3′-(((tert-butoxycarbonyl)amino)methyl)-[1,1′-biphenyl]-3-yl)ethynyl)phenyl)acetate (Example 16-B) (75 mg, 0.165 mmol) in ethyl acetate (6 mL) was reacted with Pd/C (10%) at 1 bar using the H-Cube® apparatus for 3 hrs. The solution was concentrated. This crude product was dissolved in dichloromethane (5 mL). TFA (1 mL, 12.98 mmol) was added and the mixture was stirred at room temperature for 1.5 hr, then concentrated. The crude product was dissolved in acetonitrile (2 mL) and LiOH (1 mL, 1.0 mmol) was added. The reaction mixture was stirred at room temperature overnight. The mixture was filtered and loaded directly onto reverse phase HPLC (method B). Fractions containing the desired product were pooled, the pooled fractions were frozen and lyophilized to provide the title compound. ¹H NMR (400 MHz, METHANOL-d₄) δ ppm 7.61 (s, 1H) 7.26-7.52 (m, 6H) 7.19-7.26 (m, 2H) 7.03-7.17 (m, 3H) 3.90 (s, 2H) 3.54 (s, 2H) 2.86-3.07 (m, 4H). HRMS calcd. for C₂₃H₂₃NO₂ (M+H)⁺ 346.1807. found 346.1811.

### Example 17

### Example 17-A

### Methyl 2-(2-(3-bromobenzamido)-4-chlorophenyl)acetate

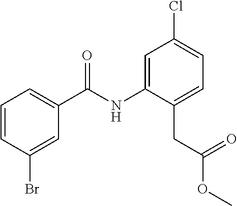 [SMILES: COC(=O)Cc1ccc(Cl)cc1NC(=O)c1cccc(Br)c1]

To a suspension of 3-bromobenzoic acid (CAS #585-76-2) (17.1 mg, 0.085 mmol) in DCM (0.85 ml) and DMF (1.32 μl, 0.017 mmol) was added oxalyl chloride (9.32 μl, 0.106 mmol) and this was stirred at room temperature. After 90 minutes additional oxalyl chloride (9.32 μl, 0.106 mmol) was added. After 5 more minutes the reaction was concentrated, and was added a solution of methyl 2-(2-amino-4-chlorophenyl)acetate (Intermediate 20-B) (17 mg, 0.085 mmol) and DIPEA (29.7 μl, 0.170 mmol) in DCM (0.85 ml). At this point the reaction was stirred at room temperature. After 80 minutes the reaction was concentrated and purified directly by flash chromatography (0-50% EtOAc:Heptanes) to provide the title compound. MS (ESI+) m/z 382.2, 384.2 (M+H).

### Example 17-B

### 2-(2-(3′-(Aminomethyl)-[1,1′-biphenyl]-3-ylcarboxamido)-4-chlorophenyl)acetic acid

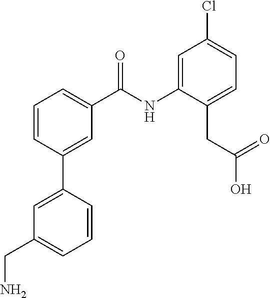 [SMILES: NCc1cccc(-c2cccc(C(=O)Nc3cc(Cl)ccc3CC(=O)O)c2)c1]

In a 2-5 mL microwave vial with stir bar was added methyl 2-(2-(3-bromobenzamido)-4-chlorophenyl)acetate (0.017 g, 0.044 mmol) and (3-(aminomethyl)phenyl)boronic acid hydrochloride (CAS #352525-94-1) (0.012 g, 0.067 mmol) and MeCN (0.800 ml) and H₂O (0.089 ml). Then, 2M aq. K₃PO₄ (0.089 ml, 0.18 mmol) and PdCl₂(dppf).CH₂Cl₂ adduct (CAS #95464-05-4) (1.8 mg, 2.2 μmol) were added. The vial was sealed and the reaction mixture was heated in a microwave at 110° C. for 1 hour. The organic layer was filtered and purified directly by preparative HPLC (method A) to provide the title compound as the TFA salt. ¹H NMR (TFA salt, 400 MHz, DMSO-d₆) δ ppm 12.44 (br. s., 1H) 10.19 (s, 1H) 8.10-8.36 (m, 4H) 7.85-8.02 (m, 3H) 7.80 (d, J=7.96 Hz, 1H) 7.67 (t, J=7.77 Hz, 1H) 7.53-7.63 (m, 2H) 7.45-7.53 (m, 1H) 7.39 (d, J=8.34 Hz, 1H) 7.31 (dd, J=8.27, 2.21 Hz, 1H) 4.06-4.22 (m, 2H) 3.72 (s, 2H). HRMS calcd. for C₂₂H₁₉ClN₂O₃(M+H)⁺ 395.1162. found 395.1154.

### Example 18

### Example 18-A

### Methyl 2-(5-bromo-2-(3′-(((tert-butoxycarbonyl)amino)methyl)-5′-fluoro-[1,1′-biphenyl]-3-ylcarboxamido)phenyl)acetate

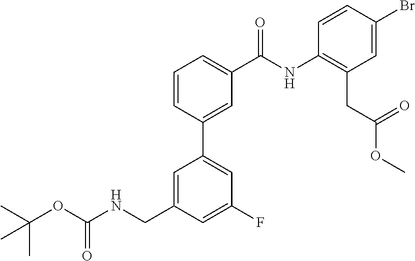 [SMILES: COC(=O)Cc1cc(Br)ccc1NC(=O)c1cccc(-c2cc(F)cc(CNC(=O)OC(C)(C)C)c2)c1]

To a solution of 3′-(((tert-butoxycarbonyl)amino)methyl)-5′-fluoro-[1,1′-biphenyl]-3-carboxylic acid (Intermediate 40-A) (54 mg, 0.16 mmol) in DCM (1.56 ml) and DMF (2.4 μl, 0.031 mmol) was added oxalyl chloride (27.4 μl, 0.313 mmol) and the reaction was stirred at room temperature. After 5 minutes the reaction was concentrated, then dissolved in DCM (1.56 ml), and methyl 2-(2-amino-5-bromophenyl)acetate (Intermediate 19) (45.8 mg, 0.188 mmol) was added followed by DIPEA (68.3 μl, 0.391 mmol), and the reaction was stirred at room temperature. After 10 minutes the reaction was purified directly by flash chromatography (0-50% EtOAc:Heptanes) followed by a second flash chromatography (0-10% EtOAc:DCM) to provide the title compound. MS (ESI−) m/z 569.5, 571.5 (M−H).

### Example 18-B

### 2-(2-(3′-(Aminomethyl)-5′-fluoro-[1,1′-biphenyl]-3-ylcarboxamido)-5-bromophenyl)acetic acid

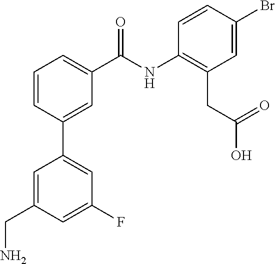 [SMILES: NCc1cc(F)cc(-c2cccc(C(=O)Nc3ccc(Br)cc3CC(=O)O)c2)c1]

To a suspension of methyl 2-(5-bromo-2-(3′-(((tert-butoxycarbonyl)amino)methyl)-5′-fluoro-[1,1′-biphenyl]-3-ylcarboxamido)phenyl)acetate (64 mg, 0.11 mmol) in diethyl ether (0.56 ml) was added HCl (2.0M in Ether, 1.12 ml, 2.24 mmol) and the reaction was stirred at 40° C. for 4 hours. The reaction was then concentrated, dissolved in THF (0.60 ml), MeOH (0.20 ml) and water (0.20 ml), and LiOH.H₂O (14.1 mg, 0.336 mmol) was added. After 5 minutes the reaction was filtered and purified by preparative HPLC (Method B) to provide the title compound. ¹H NMR (400 MHz, METHANOL-d₄) δ ppm 8.44 (t, J=1.64 Hz, 1H) 8.10 (d, J=8.46 Hz, 1H) 7.85-7.95 (m, 2H) 7.70 (s, 1H) 7.63 (t, J=7.83 Hz, 1H) 7.35-7.52 (m, 3H) 7.17 (d, J=9.73 Hz, 1H) 4.05 (s, 2H) 3.58 (s, 2H). HRMS calcd. for C₂₂H₁₈BrFN₂O₃(M+H)⁺ 457.0563 and 459.0543. found 457.0556 and 459.0540.

### Example 19

### Example 19-A

### (±)-tert-Butyl 2-(2-(3′-(((tert-butoxycarbonyl)amino)methyl)-5′-fluoro-[1,1′-biphenyl]-3-ylcarboxamido)phenyl)propanoate

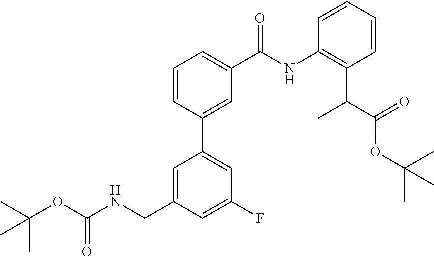 [SMILES: CC(C(=O)OC(C)(C)C)c1ccccc1NC(=O)c1cccc(-c2cc(F)cc(CNC(=O)OC(C)(C)C)c2)c1]

To a solution of 3′-(((tert-butoxycarbonyl)amino)methyl)-5′-fluoro-[1,1′-biphenyl]-3-carboxylic acid (Intermediate 40-A) (122 mg, 0.352 mmol) in DCM (3.52 ml) and DMF (5.46 μl, 0.070 mmol) was added oxalyl chloride (61.7 μl, 0.705 mmol) and this was stirred at room temperature. After 5 minutes the reaction was concentrated, it was dissolved in DCM (3.52 ml), and tert-butyl 2-(2-aminophenyl)propanoate (Intermediate 16-C) (78 mg, 0.35 mmol) was added followed by DIPEA (154 μl, 0.881 mmol) and the reaction was stirred at room temperature. After 10 minutes the mixture was concentrated and purified directly by flash chromatography (0-10% EtOAc:DCM) to provide the title compound. MS (ESI−) m/z 547.6 (M−H).

### Example 19-B

### (±)-2-(2-(3′-(Aminomethyl)-5′-fluoro-[1,1′-biphenyl]-3-ylcarboxamido)phenyl)propanoic acid

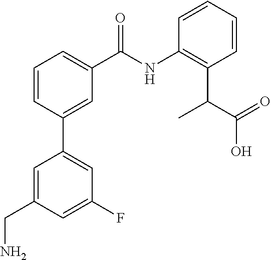 [SMILES: CC(C(=O)O)c1ccccc1NC(=O)c1cccc(-c2cc(F)cc(CN)c2)c1]

To a suspension of tert-butyl 2-(2-(3′-(((tert-butoxycarbonyl)amino)methyl)-5′-fluoro-[1,1′-biphenyl]-3-ylcarboxamido)phenyl)propanoate (109 mg, 0.199 mmol) in dioxane (3.97 ml) was added HCl (4.0M in Dioxane, 1.49 ml, 5.96 mmol) and the reaction was stirred at 60° C. After stirring overnight the reaction was concentrated and purified by preparative HPLC (Method B) to provide the title compound. ¹H NMR (400 MHz, METHANOL-d₄) δ ppm 8.53 (s, 1H) 8.07-8.16 (m, 1H) 7.86-7.95 (m, 2H) 7.84 (s, 1H) 7.65 (t, J=7.83 Hz, 1H) 7.55 (ddd, J=9.98, 2.02, 1.89 Hz, 1H) 7.35 (dd, J=7.58, 1.26 Hz, 1H) 7.10-7.32 (m, 3H) 4.21 (s, 2H) 3.82 (q, J=7.16 Hz, 1H) 1.50 (d, J=7.33 Hz, 3H). HRMS calcd. for C₂₃H₂₁FN₂O₃(M+H)⁺ 393.1614. found 393.1601.

### Example 20

### Example 20-A

### tert-Butyl 2-(2-(6-(3-(((tert-butoxycarbonyl)amino)methyl)-5-fluorophenyl)picolinamido)phenyl)acetate

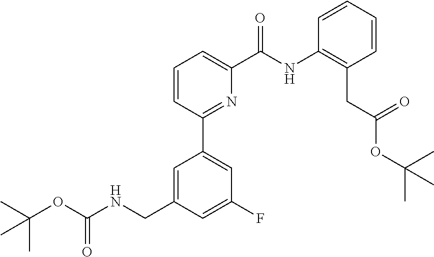 [SMILES: CC(C)(C)OC(=O)Cc1ccccc1NC(=O)c1cccc(-c2cc(F)cc(CNC(=O)OC(C)(C)C)c2)n1]

The title compound was synthesized as described in Example 19-A starting with 6-(3-(((tert-butoxycarbonyl)amino)methyl)-5-fluorophenyl)picolinic acid (Intermediate 40-B) and tert-butyl 2-(2-aminophenyl)acetate (CAS #98911-34-3), and using (0-20% EtOAc:DCM) for the flash chromatography. MS (ESI−) m/z 534.5 (M−H).

### Example 20-B

### 2-(2-(6-(3-(Aminomethyl)-5-fluorophenyl)picolinamido)phenyl)acetic acid

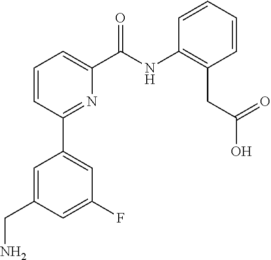 [SMILES: NCc1cc(F)cc(-c2cccc(C(=O)Nc3ccccc3CC(=O)O)n2)c1]

Tert-butyl 2-(2-(6-(3-(((tert-butoxycarbonyl)amino)methyl)-5-fluorophenyl)picolinamido)phenyl)acetate (68 mg, 0.13 mmol) was dissolved in DCM (1.0 ml) and TFA (1.0 ml) and stirred at room temperature. After 30 minutes the reaction was concentrated and purified by preparative HPLC (Method B) to provide the title compound. ¹H NMR (400 MHz, DMSO-d₆) δ ppm 12.20 (s, 1H) 9.20 (s, 1H) 8.37 (dd, J=7.45, 1.52 Hz, 1H) 8.07-8.22 (m, 2H) 8.02 (d, J=9.98 Hz, 1H) 7.78-7.90 (m, 1H) 7.36 (d, J=9.22 Hz, 1H) 7.16-7.31 (m, 2H) 7.01-7.14 (m, 1H) 4.11 (s, 2H) 3.41 (s, 2H). HRMS calcd. for C₂₁H₁₈FN₃O₃ (M+H)⁺ 380.1410. found 380.1418.

### Example 21

The following compounds were prepared with similar methods as described in Examples 18-20 using the anilines from Intermediates 1-19 and the carboxylic acids from Intermediates 40.

| | | Method of | | |
|---|---|---|---|---|
| | Structure/Chemical Name | deprotection | ¹H NMR | HRMS |
| 21-A | 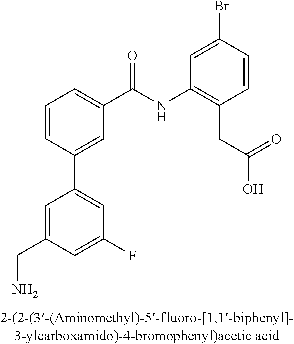 [SMILES: NCc1cc(F)cc(-c2cccc(C(=O)Nc3cc(Br)ccc3CC(=O)O)c2)c1] | 18-B | (400 MHz, METHANOL-d₄) δ ppm 8.46 (s, 1 H) 8.21 (d, J = 1.77 Hz, 1 H) 8.11 (d, J = 7.71 Hz, 1 H) 7.90 (d, J = 7.83 Hz, 1 H) 7.73 (s, 1 H) 7.64 (t, J = 7.77 Hz, 1 H) 7.47 (d, J = 9.85 Hz, 1 H) 7.12-7.31 (m, 3 H) 4.08 (s, 2 H) 3.58 (s, 2 H). | MS (ESI+) m/z 457.4, 459.4 (M + H). |
| 21-B | 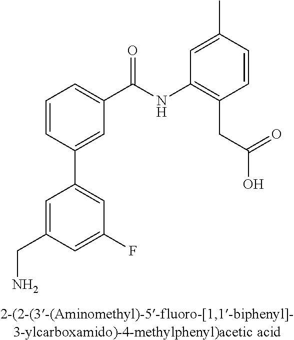 [SMILES: Cc1ccc(CC(=O)O)c(NC(=O)c2cccc(-c3cc(F)cc(CN)c3)c2)c1] | 18-B (except Dioxane and HCl/Dioxane were used instead of Ether and HCl/Ether) | (400 MHz, METHANOL-d₄) δ ppm 8.49 (s, 1 H) 8.09 (d, J = 7.83 Hz, 1 H) 7.82-7.94 (m, 2 H) 7.78 (s, 1 H) 7.65 (t, J = 7.77 Hz, 1 H) 7.55 (d, J = 9.85 Hz, 1 H) 7.14-7.28 (m, 2 H) 6.95 (d, J = 6.69 Hz, 1 H) 4.23 (s, 2 H) 3.50-3.62 (m, 2 H) 2.36 (s, 3 H). | calcd. for C₂₃H₂₁FN₂O₃ (M + H)⁺ 393.1614, found 393.1609. |
| 21-C | 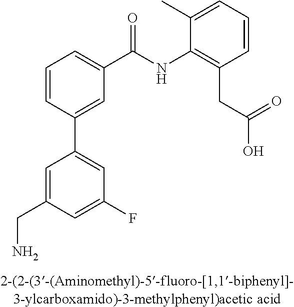 [SMILES: Cc1cccc(CC(=O)O)c1NC(=O)c1cccc(-c2cc(F)cc(CN)c2)c1] | 19-B | (400 MHz, METHANOL-d₄) δ ppm 8.45 (t, J = 1.71 Hz, 1 H) 8.04 (dt, J = 8.02, 1.23 Hz, 1 H) 7.87-7.95 (m, 1 H) 7.83 (s, 1 H) 7.65 (t, J = 7.77 Hz, 1 H) 7.56 (dt, J = 9.76, 2.01 Hz, 1 H) 7.11-7.27 (m, 4 H) 4.23 (s, 2 H) 3.58 (br. s., 2 H) 2.32 (s, 3 H) | calcd. for C₂₃H₂₁FN₂O₃ (M + H)⁺ 393.1614, found 393.1609. |
| 21-D | 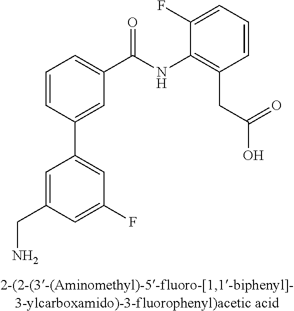 [SMILES: NCc1cc(F)cc(-c2cccc(C(=O)Nc3c(F)cccc3CC(=O)O)c2)c1] | 19-B | (400 MHz, METHANOL-d₄) δ ppm 8.33 (s, 1 H) 8.04 (d, J = 7.45 Hz, 1 H) 7.92 (d, J = 8.59 Hz, 1 H) 7.71 (s, 1 H) 7.66 (t, J = 7.77 Hz, 1 H) 7.57 (d, J = 9.98 Hz, 1 H) 7.07-7.36 (m, 4 H) 4.23 (s, 2 H) 3.68 (s, 2 H). | calcd. for C₂₂H₁₈F₂N₂O₃ (M + H)⁺ 397.1364, found 397.1352. |
| 21-E | 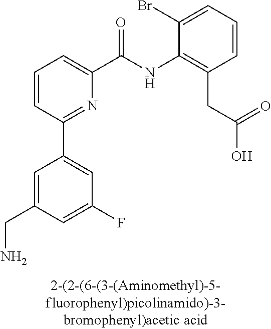 [SMILES: NCc1cc(F)cc(-c2cccc(C(=O)Nc3c(Br)cccc3CC(=O)O)n2)c1] | 19-B | (400 MHz, METHANOL-d₄) δ ppm 8.96 (s, 1 H) 8.22 (dd, J = 7.45, 1.52 Hz, 1 H) 8.06-8.19 (m, 2 H) 7.81-7.90 (m, 1 H) 7.60 (dd, J = 8.08, 1.39 Hz, 1 H) 7.32 (dd, J = 7.58, 1.39 Hz, 1 H) 7.22-7.28 (m, 1 H) 7.13-7.22 (m, 1 H) 4.25 (s, 2 H) 3.62 (s, 2 H). | calcd. for C₂₁H₁₇BrFN₃O₃ (M + H)⁺ 458.0516 and 460.0496, found 458.0500 and 460.0481. |
| 21-F | 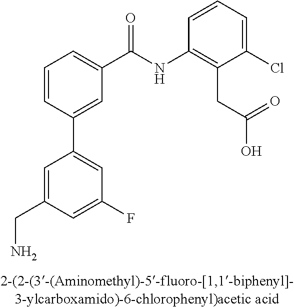 [SMILES: NCc1cc(F)cc(-c2cccc(C(=O)Nc3cccc(Cl)c3CC(=O)O)c2)c1] | 19-B | (400 MHz, METHANOL-d₄) δ ppm 8.36 (s, 1 H) 8.08 (d, J = 8.34 Hz, 1 H) 7.75-7.93 (m, 2 H) 7.62 (t, J = 7.77 Hz, 1 H) 7.56 (s, 1 H) 7.34 (d, J = 9.60 Hz, 1 H) 7.18-7.30 (m, 2 H) 7.12 (d, J = 9.47 Hz, 1 H) 3.88 (s, 2 H) 3.83 (s, 2 H). | calcd. for C₂₂H₁₈ClFN₂O₃ (M + H)⁺ 413.1068, found 413.1059. |
| 21-G | 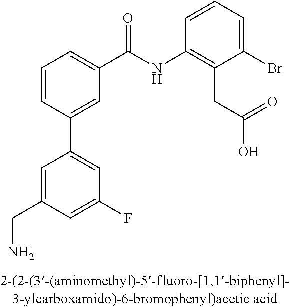 [SMILES: NCc1cc(F)cc(-c2cccc(C(=O)Nc3cccc(Br)c3CC(=O)O)c2)c1] | 19-B | (400 MHz, METHANOL-d₄) δ ppm 8.43 (s, 1 H) 8.08 (d, J = 8.08 Hz, 1 H) 7.91 (t, J = 7.77 Hz, 2 H) 7.83 (s, 1 H) 7.60-7.69 (m, 1 H) 7.55 (d, J = 9.98 Hz, 1 H) 7.45 (d, J = 8.21 Hz, 1 H) 7.15-7.24 (m, 2 H) 4.23 (s, 2 H) 3.93 (s, 2 H). | calcd. for C₂₂H₁₈BrFN₂O₃ (M + H)⁺ 457.0563 and 459.0543, found 457.0557 and 459.0540. |
| 21-H | 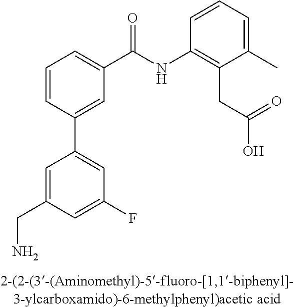 [SMILES: Cc1cccc(NC(=O)c2cccc(-c3cc(F)cc(CN)c3)c2)c1CC(=O)O] | 19-B | (400 MHz, METHANOL-d₄) δ ppm 8.49 (s, 1 H) 8.09 (d, J = 7.83 Hz, 1 H) 7.91 (d, J = 8.08 Hz, 1 H) 7.84 (s, 1 H) 7.72 (d, J = 7.70 Hz, 1 H) 7.65 (t, J = 7.77 Hz, 1 H) 7.55 (ddd, J = 9.85, 2.02, 1.89 Hz, 1 H) 7.11-7.26 (m, 2 H) 7.04 (d, J = 7.45 Hz, 1 H) 4.56 (br. s., 2 H) 4.23 (s, 2 H) 3.66 (s, 2 H) 2.46 (s, 3 H). | calcd. for C₂₃H₂₁FN₂O₃ (M + H)⁺ 393.1614, found 393.1616. |
| 21-I | 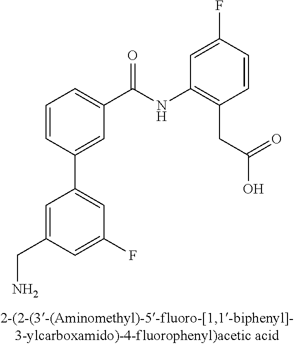 [SMILES: NCc1cc(F)cc(-c2cccc(C(=O)Nc3cc(F)ccc3CC(=O)O)c2)c1] | 19-B | (400 MHz, METHANOL-d₄) δ ppm 8.48-8.55 (m, 1 H) 8.11 (dt, J = 7.80, 1.41 Hz, 1H) 7.88-7.95 (m, 1 H) 7.78-7.86 (m, 2 H) 7.65 (t, J = 7.77 Hz, 1 H) 7.50-7.58 (m, 1 H) 7.29 (dd, J = 8.46, 6.32 Hz, 1 H) 7.21 (d, J = 9.22 Hz, 1 H) 6.86 (td, J = 8.40, 2.78 Hz, 1 H) 4.21 (s, 2 H) 3.60 (s, 2 H). | MS (ESI+) m/z 397.4 (M + H). |
| 21-J | 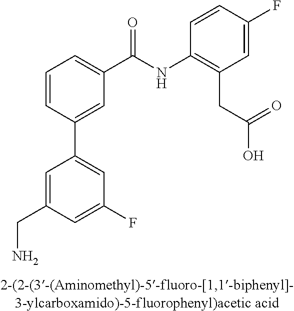 [SMILES: NCc1cc(F)cc(-c2cccc(C(=O)Nc3ccc(F)cc3CC(=O)O)c2)c1] | 19-B | (400 MHz, METHANOL-d₄) δ ppm 8.48 (s, 1 H) 8.05-8.13 (m, 1 H) 7.88-7.98 (m, 2 H) 7.83 (s, 1 H) 7.65 (t, J = 7.77 Hz, 1 H) 7.55 (dt, J = 9.82, 1.91 Hz, 1 H) 7.22 (d, J = 8.84 Hz, 1 H) 6.94-7.12 (m, 2 H) 4.22 (s, 2 H) 3.61 (s, 2 H). | calcd. for C₂₂H₁₈F₂N₂O₃ (M + H)⁺ 397.1364, found 397.1350. |
| 21-K | 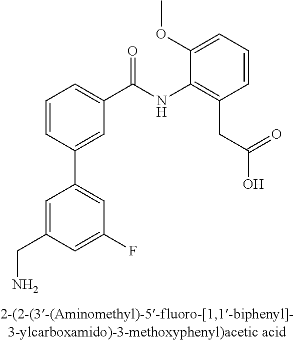 [SMILES: COc1cccc(CC(=O)O)c1NC(=O)c1cccc(-c2cc(F)cc(CN)c2)c1] | 19-B | (400 MHz, METHANOL-d₄) δ ppm 8.38 (s, 1 H) 8.02 (d, J = 7.83 Hz, 1 H) 7.90 (d, J = 7.71 Hz, 1 H) 7.79 (s, 1 H) 7.64 (t, J = 7.83 Hz, 1 H) 7.55 (d, J = 9.98 Hz, 1 H) 7.16-7.28 (m, 2 H) 7.00 (d, J = 8.21 Hz, 1 H) 6.94 (d, J = 7.71 Hz, 1 H) 4.22 (s, 2 H) 3.85 (s, 3 H) 3.57 (s, 2 H). | calcd. for C₂₃H₂₁FN₂O₄ (M + H)⁺ 409.1564, found 409.1550. |
| 21-L | 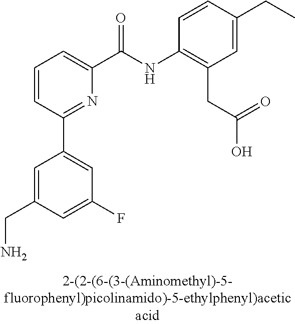 [SMILES: CCc1ccc(NC(=O)c2cccc(-c3cc(F)cc(CN)c3)n2)c(CC(=O)O)c1] | 19-B | (400 MHz, METHANOL-d₄) δ ppm 8.98 (s, 1 H) 8.15-8.24 (m, 2 H) 8.07-8.15 (m, 1 H) 7.85 (dt, J = 10.07, 1.91 Hz, 1 H) 7.73-7.81 (m, 1 H) 7.25 (dd, J = 8.72, 1.89 Hz, 1 H) 7.12-7.19 (m, 2 H) 4.25 (s, 2 H) 3.60 (s, 2 H) 2.65 (q, J = 7.58 Hz, 2 H) 1.15-1.34 (m, 3 H). | calcd. for C₂₃H₂₂FN₃O₃ (M + H)⁺ 408.1723, found 408.1710. |
| 21-M | 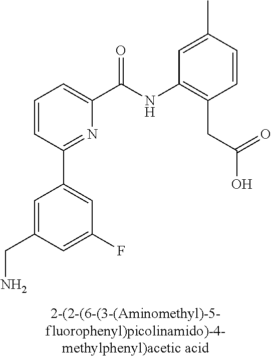 [SMILES: Cc1ccc(CC(=O)O)c(NC(=O)c2cccc(-c3cc(F)cc(CN)c3)n2)c1] | 19-B | (400 MHz, METHANOL-d₄) δ ppm 9.00 (s, 1 H) 8.20 (dd, J = 8.15, 0.95 Hz, 2 H) 8.06-8.15 (m, 1 H) 7.84 (dt, J = 10.04, 1.93 Hz, 1 H) 7.71 (s, 1 H) 7.24 (d, J = 8.72 Hz, 1 H) 7.18 (d, J = 7.71 Hz, 1 H) 6.95-7.02 (m, 1 H) 4.24 (s, 2 H) 3.56 (s, 2 H) 2.37 (s, 3 H). | calcd. for C₂₂H₂₀FN₃O₃ (M + H)⁺ 394.1567, found 394.1566. |
| 21-N | 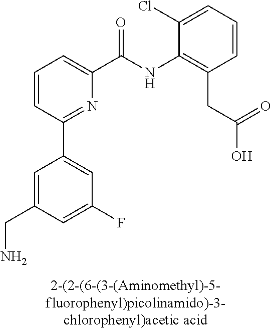 [SMILES: NCc1cc(F)cc(-c2cccc(C(=O)Nc3c(Cl)cccc3CC(=O)O)n2)c1] | 19-B | (400 MHz, METHANOL-d₄) δ ppm 8.99 (s, 1 H) 8.23 (dd, J = 7.45, 1.39 Hz, 1 H) 8.07-8.19 (m, 2 H) 7.86 (dt, J = 9.95, 1.91 Hz, 1 H) 7.36-7.48 (m, 1 H) 7.19-7.31 (m, 3 H) 4.26 (s, 2 H) 3.62 (br. s., 2 H). | calcd. for C₂₁H₁₇ClFN₃O₃ (M + H)⁺ 414.1021, found 414.1013. |
| 21-O | 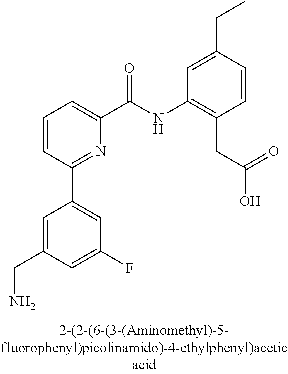 [SMILES: CCc1ccc(CC(=O)O)c(NC(=O)c2cccc(-c3cc(F)cc(CN)c3)n2)c1] | 20-B | (400 MHz, METHANOL-d₄) δ ppm 8.99 (s, 1 H) 8.15-8.24 (m, 2 H) 8.07-8.15 (m, 1 H) 7.84 (dt, J = 9.95, 1.85 Hz, 1 H) 7.74 (d, J = 1.64 Hz, 1 H) 7.17-7.27 (m, 2 H) 7.02 (dd, J = 7.71, 1.77 Hz, 1 H) 4.24 (s, 2 H) 3.57 (s, 2 H) 2.68 (q, J = 7.66 Hz, 2 H) 1.27 (t, J = 7.64 Hz, 3 H). | calcd. for C₂₃H₂₂FN₃O₃ (M + H)⁺ 408.1723, found 408.1712. |
| 21-P | 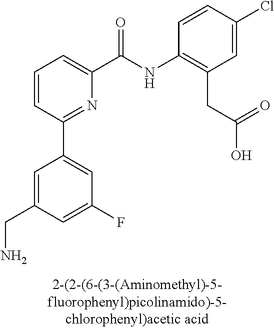 [SMILES: NCc1cc(F)cc(-c2cccc(C(=O)Nc3ccc(Cl)cc3CC(=O)O)n2)c1] | 20-B | (400 MHz, DMSO-d₆) δ ppm 12.19 (s, 1 H) 9.10-9.49 (m, 3 H) 8.38 (d, J = 6.57 Hz, 1 H) 8.08-8.22 (m, 2 H) 8.02 (d, J = 10.36 Hz, 1 H) 7.86 (d, J = 8.72 Hz, 1 H) 7.24-7.44 (m, 3 H) 4.12 (s, 2 H) 3.42 (s, 2 H). | calcd. for C₂₁H₁₇ClFN₃O₃ (M + H)⁺ 414.1021, found 414.1014. |
| 21-Q | 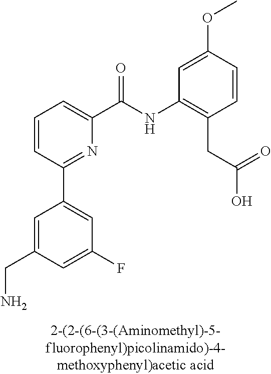 [SMILES: COc1ccc(CC(=O)O)c(NC(=O)c2cccc(-c3cc(F)cc(CN)c3)n2)c1] | 20-B | (400 MHz, DMSO-d₆) δ ppm 12.17 (br. s., 1 H) 9.11-9.52 (m, 3 H) 8.37 (dd, J = 7.52, 1.45 Hz, 1 H) 8.08-8.22 (m, 2 H) 8.02 (d, J = 9.85 Hz, 1 H) 7.48 (d, J = 2.78 Hz, 1 H) 7.36 (d, J = 9.73 Hz, 1 H) 7.11 (d, J = 8.34 Hz, 1 H) 6.68 (dd, J = 8.34, 2.65 Hz, 1 H) 4.11 (s, 2 H) 3.75 (s, 3 H) 3.34 (br. s., 2 H). | calcd. for C₂₂H₂₀FN₃O₄ (M + H)⁺ 410.1516, found 410.1517. |

### Example 22

### Example 22-A

### tert-Butyl 2-(5-bromo-2-(3′-(((tert-butoxycarbonyl)amino)methyl)-5′-fluoro-[1,1′-biphenyl]-3-ylcarboxamido)phenyl)acetate

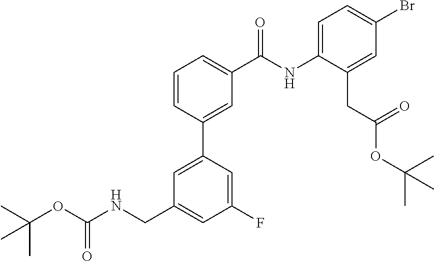 [SMILES: CC(C)(C)OC(=O)Cc1cc(Br)ccc1NC(=O)c1cccc(-c2cc(F)cc(CNC(=O)OC(C)(C)C)c2)c1]

The title compound was synthesized as described in Example 18-A starting with 3′-(((tert-butoxycarbonyl)amino)methyl)-5′-fluoro-[1,1′-biphenyl]-3-carboxylic acid (Intermediate 40-A) and tert-butyl 2-(2-amino-5-bromophenyl)acetate (Intermediate 14-A). MS (ESI−) m/z 611.7, 613.6 (M−H).

### Example 22-B

### tert-Butyl 2-(2-(3′-(((tert-butoxycarbonyl)amino)methyl)-5′-fluoro-[1,1′-biphenyl]-3-ylcarboxamido)-5-methylphenyl)acetate

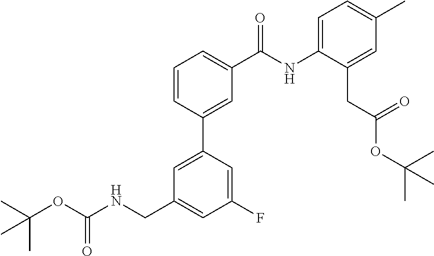 [SMILES: Cc1ccc(NC(=O)c2cccc(-c3cc(F)cc(CNC(=O)OC(C)(C)C)c3)c2)c(CC(=O)OC(C)(C)C)c1]

The title compound was synthesized as described in Intermediate 13-A starting with tert-butyl 2-(5-bromo-2-(3′-(((tert-butoxycarbonyl)amino)methyl)-5′-fluoro-[1,1′-biphenyl]-3-ylcarboxamido)phenyl)acetate (Example 22-A), (0-10% EtOAc:DCM) was used for the flash chromatography. MS (ESI−) m/z 547.6 (M−H).

### Example 22-C

### 2-(2-(3′-(aminomethyl)-5′-fluoro-[1,1′-biphenyl]-3-ylcarboxamido)-5-methylphenyl)acetic acid

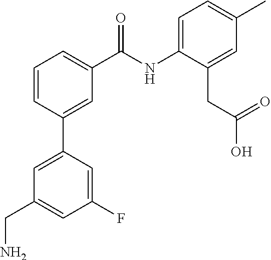 [SMILES: Cc1ccc(NC(=O)c2cccc(-c3cc(F)cc(CN)c3)c2)c(CC(=O)O)c1]

The title compound was synthesized as described in Example 19-B from tert-butyl 2-(2-(3′-(((tert-butoxycarbonyl)amino)methyl)-5′-fluoro-[1,1′-biphenyl]-3-ylcarboxamido)-5-methylphenyl)acetate. ¹H NMR (400 MHz, METHANOL-d₄) δ ppm 8.49 (s, 1H) 8.09 (d, J=7.71 Hz, 1H) 7.90 (d, J=8.21 Hz, 1H) 7.85 (s, 1H) 7.81 (d, J=8.34 Hz, 1H) 7.64 (t, J=7.83 Hz, 1H) 7.55 (d, J=9.85 Hz, 1H) 7.22 (d, J=8.84 Hz, 1H) 7.06-7.16 (m, 2H) 4.23 (s, 2H) 3.58 (s, 2H) 2.33 (s, 3H). HRMS calcd. for C₂₃H₂₁FN₂O₃(M+H)⁺ 393.1614. found 393.1608.

### Example 23

### Example 23-A

### tert-Butyl 2-(2-(3′-(((tert-butoxycarbonyl)amino)methyl)-5′-fluoro-[1,1′-biphenyl]-3-ylcarboxamido)-5-vinylphenyl)acetate

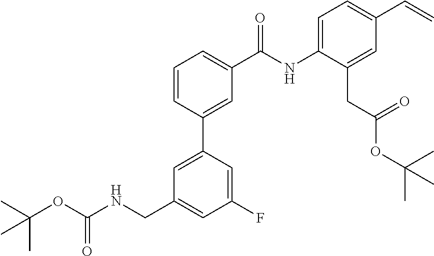 [SMILES: C=Cc1ccc(NC(=O)c2cccc(-c3cc(F)cc(CNC(=O)OC(C)(C)C)c3)c2)c(CC(=O)OC(C)(C)C)c1]

In a microwave vial, tert-butyl 2-(5-bromo-2-(3′-(((tert-butoxycarbonyl)amino)methyl)-5′-fluoro-[1,1′-biphenyl]-3-ylcarboxamido)phenyl)acetate (Example 22-A) (0.120 g, 0.196 mmol) was dissolved in DME (1.47 ml) and water (0.49 ml). Vinylboronic anhydride pyridine complex (CAS #442850-89-7) (0.061 g, 0.25 mmol) and Pd(PPh₃)₄ (0.023 g, 0.020 mmol) and K₂CO₃ (0.027 g, 0.20 mmol) were added and the reaction was heated in the microwave at 100° C. for 1 hour. The reaction was diluted with water, extracted with EtOAc, dried with MgSO₄, filtered and concentrated. The crude residue was purified by flash chromatography (0-100% EtOAc:Heptanes) to provide the title compound. MS (ESI−) m/z 559.5 (M−H).

### Example 23-B

### tert-Butyl 2-(2-(3′-(((tert-butoxycarbonyl)amino)methyl)-5′-fluoro-[1,1′-biphenyl]-3-ylcarboxamido)-5-ethylphenyl)acetate

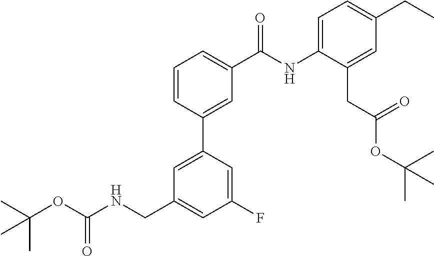 [SMILES: CCc1ccc(NC(=O)c2cccc(-c3cc(F)cc(CNC(=O)OC(C)(C)C)c3)c2)c(CC(=O)OC(C)(C)C)c1]

The title compound was synthesized as described in Intermediate 1-B (except using EtOAc instead of EtOH as the solvent), starting from tert-butyl 2-(2-(3′-(((tert-butoxycarbonyl)amino)methyl)-5′-fluoro-[1,1′-biphenyl]-3-ylcarboxamido)-5-vinylphenyl)acetate. MS (ESI−) m/z 561.6 (M−H).

### Example 23-C

### 2-(2-(3′-(Aminomethyl)-5′-fluoro-[1,1′-biphenyl]-3-ylcarboxamido)-5-ethylphenyl)acetic acid

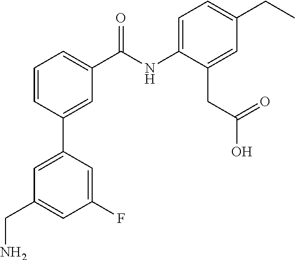 [SMILES: CCc1ccc(NC(=O)c2cccc(-c3cc(F)cc(CN)c3)c2)c(CC(=O)O)c1]

The title compound was synthesized as described in Example 19-B starting with tert-butyl 2-(2-(3′-(((tert-butoxycarbonyl)amino)methyl)-5′-fluoro-[1,1′-biphenyl]-3-ylcarboxamido)-5-ethylphenyl)acetate. ¹H NMR (400 MHz, METHANOL-d₄) δ ppm 8.49 (s, 1H) 8.09 (ddd, J=8.05, 1.36, 1.07 Hz, 1H) 7.86-7.94 (m, 1H) 7.79-7.86 (m, 2H) 7.64 (t, J=7.77 Hz, 1H) 7.55 (dd, J=9.85, 1.77 Hz, 1H) 7.21 (d, J=8.84 Hz, 1H) 7.05-7.18 (m, 2H) 4.22 (s, 2H) 3.60 (s, 2H) 2.64 (q, J=7.58 Hz, 2H) 1.24 (t, J=7.58 Hz, 3H). HRMS calcd. for C₂₄H₂₃FN₂O₃(M+H)⁺ 407.1771. found 407.1757.

### Example 24

### 2-(2-(3′-(Aminomethyl)-5′-fluoro-[1,1′-biphenyl]-3-ylcarboxamido)-5-(1-hydroxyethyl)phenyl)acetic acid

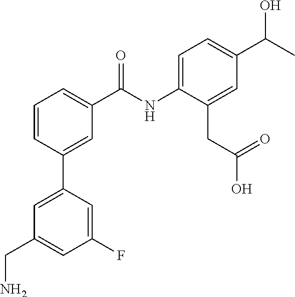 [SMILES: CC(O)c1ccc(NC(=O)c2cccc(-c3cc(F)cc(CN)c3)c2)c(CC(=O)O)c1]

The title compound was synthesized as described in Example 19-B starting with tert-butyl 2-(2-(3′-(((tert-butoxycarbonyl)amino)methyl)-5′-fluoro-[1,1′-biphenyl]-3-ylcarboxamido)-5-vinylphenyl)acetate (Example 23-A). ¹H NMR (400 MHz, DMSO-d₆) δ ppm 12.68 (br. s., 1H) 8.69 (s, 1H) 8.13 (s, 1H) 8.00 (dd, J=9.98, 8.97 Hz, 2H) 7.91 (d, J=8.08 Hz, 1H) 7.57-7.77 (m, 2H) 7.30 (d, J=9.35 Hz, 1H) 7.11-7.24 (m, 2H) 5.09 (br. s., 1H) 4.69 (d, J=4.55 Hz, 1H) 4.15 (s, 2H) 3.52 (s, 2H) 1.32 (d, J=6.32 Hz, 3H). HRMS calcd. for C₂₄H₂₃FN₂O₄(M+H)⁺ 423.1720. found 423.1705.

### Example 25-A

### tert-Butyl 2-(2-(3-bromo-5-iodobenzamido)-5-ethylphenyl)acetate

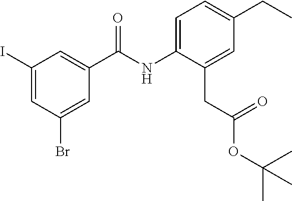 [SMILES: CCc1ccc(NC(=O)c2cc(Br)cc(I)c2)c(CC(=O)OC(C)(C)C)c1]

Triethylamine (0.367 ml, 2.63 mmol) was added to a mixture of 3-bromo-5-iodobenzoic acid (CAS #188815-32-9) (0.452 g, 1.38 mmol), tert-butyl 2-(2-amino-5-ethylphenyl)acetate (Intermediate 14-C) (0.310 g, 1.32 mmol) and HATU (0.526 g, 1.38 mmol) in DMF (6.59 ml) at room temperature. After 2 hours the mixture was partitioned between 1:1 EtOAc/heptane and water. The aqueous layer was extracted with 1:1 EtOAc/heptane. The combined organic layers were washed with brine, dried over Na₂SO₄, filtered and concentrated. The crude product was purified by flash chromatography (0-20% EtOAc:Heptanes) to provide the title compound. MS (ESI−) m/z 542.2, 544.2 (M−H).

### Example 25-B

### tert-Butyl 2-(2-(5-bromo-3′-(((tert-butoxycarbonyl)amino)methyl)-5′-fluoro-[1,1′-biphenyl]-3-ylcarboxamido)-5-ethylphenyl)acetate

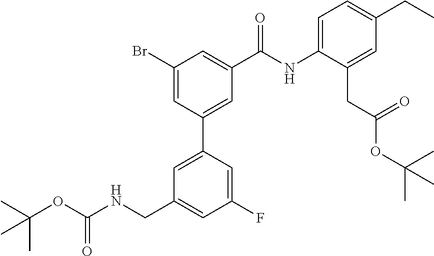 [SMILES: CCc1ccc(NC(=O)c2cc(Br)cc(-c3cc(F)cc(CNC(=O)OC(C)(C)C)c3)c2)c(CC(=O)OC(C)(C)C)c1]

A degassed mixture of tert-butyl 2-(2-(3-bromo-5-iodobenzamido)-5-ethylphenyl)acetate (0.063 g, 0.12 mmol), tert-butyl 3-fluoro-5-(4,4,5,5-tetramethyl-1,3,2-dioxaborolan-2-yl)benzylcarbamate (Intermediate 24) (0.041 g, 0.093 mmol), PdCl₂(dppf).CH₂Cl₂ adduct (CAS #95464-05-4) (4.73 mg, 5.79 μmol), and 2M aqueous K₃PO₄ (0.116 ml, 0.232 mmol) in DMF (1.16 ml) was heated at 50° C. for 2.5 hours. The reaction mixture was diluted with EtOAc and washed with water. The aqueous layer was extracted with EtOAc. The combined organic layers were washed with brine, dried with Na₂SO₄, filtered and concentrated. The crude was purified by flash chromatography (0-50% EtOAc:Heptanes) to provide the title compound. MS (ESI−) m/z 639.6, 641.5 (M−H).

### Example 25-C

### (S)-tert-Butyl 2-(2-(3′-(((tert-butoxycarbonyl)amino)methyl)-5′-fluoro-5-(((tetrahydrofuran-2-yl)methyl)amino)-[1,1′-biphenyl]-3-ylcarboxamido)-5-ethylphenyl)acetate

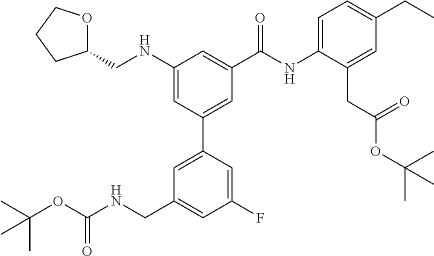 [SMILES: CCc1ccc(NC(=O)c2cc(NC[C@@H]3CCCO3)cc(-c3cc(F)cc(CNC(=O)OC(C)(C)C)c3)c2)c(CC(=O)OC(C)(C)C)c1]

A suspension of tert-butyl 2-(2-(5-bromo-3′-(((tert-butoxycarbonyl)amino)methyl)-5′-fluoro-[1,1′-biphenyl]-3-ylcarboxamido)-5-ethylphenyl)acetate (0.190 g, 0.296 mmol), (S)-(tetrahydrofuran-2-yl)methanamine (CAS #7175-81-7) (0.030 g, 0.30 mmol), Cs₂CO₃ (0.289 g, 0.888 mmol) and BrettPhos palladacycle (CAS #1148148-01-9) (0.012 g, 0.015 mmol) in acetonitrile (2.96 ml) was heated at 110° C. for 60 minutes. The solution was diluted with EtOAc and water, extracted with EtOAc, dried with MgSO₄, filtered and concentrated. The crude was purified by flash chromatography (0-80% EtOAc:Heptanes) to provide the title compound. MS (ESI−) m/z 660.8 (M−H).

### Example 25-D

### (S)-2-(2-(3′-(Aminomethyl)-5′-fluoro-5-(((tetrahydrofuran-2-yl)methyl)amino)-[1,1′-biphenyl]-3-ylcarboxamido)-5-ethylphenyl)acetic acid

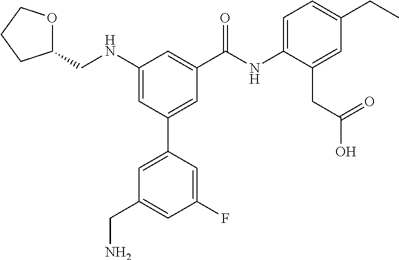 [SMILES: CCc1ccc(NC(=O)c2cc(NC[C@@H]3CCCO3)cc(-c3cc(F)cc(CN)c3)c2)c(CC(=O)O)c1]

The title compound was synthesized as described in Example 20-B from (S)-tert-butyl 2-(2-(3′-(((tert-butoxycarbonyl)amino)methyl)-5′-fluoro-5-(((tetrahydrofuran-2-yl)methyl)amino)-[1,1′-biphenyl]-3-ylcarboxamido)-5-ethylphenyl)acetate. ¹H NMR (400 MHz, METHANOL-d₄) δ ppm 7.75-7.87 (m, 2H) 7.69 (t, J=1.39 Hz, 1H) 7.47 (dt, J=9.88, 1.94 Hz, 1H) 7.31-7.36 (m, 1H) 7.06-7.21 (m, 4H) 4.20 (s, 2H) 4.12-4.19 (m, 1H) 3.86-3.97 (m, 1H) 3.78 (td, J=7.74, 6.38 Hz, 1H) 3.58 (s, 2H) 2.63 (q, J=7.58 Hz, 2H) 2.04-2.17 (m, 1H) 1.85-2.04 (m, 2H) 1.74 (ddt, J=12.03, 8.56, 7.07, 7.07 Hz, 1H) 1.24 (t, J=7.64 Hz, 3H). HRMS calcd. for C₂₉H₃₂FN₃O₄(M+H)⁺ 506.2455. found 506.2436.

### Example 26

### Example 26-A

### Methyl 2-(2-(4-bromothiazole-2-carboxamido)phenyl)acetate

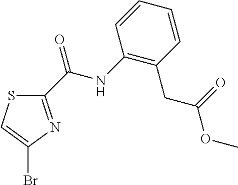 [SMILES: COC(=O)Cc1ccccc1NC(=O)c1nc(Br)cs1]

To a suspension of 4-bromothiazole-2-carboxylic acid (CAS #88982-82-5) (0.112 g, 0.538 mmol) in DCM (5.38 mL) and DMF (8.34 μL, 0.108 mmol) was added oxalyl chloride (0.059 mL, 0.67 mmol) and this was stirred at room temperature. After 30 minutes the reaction was concentrated. The solid was dissolved in DCM (5.38 mL), and methyl 2-(2-aminophenyl)acetate hydrochloride (CAS #35613-44-6) (0.089 g, 0.538 mmol) and DIPEA (0.188 mL, 1.077 mmol) were added and the reaction was stirred at room temperature. After 5 minutes the reaction was partially concentrated and then purified directly by flash chromatography (0-50% EtOAc:Heptanes) to provide the title compound. MS (ESI−) m/z 353.1, 355.1 (M−H).

### Example 26-B

### 2-(2-(4-(3-(Aminomethyl)phenyl)thiazole-2-carboxamido)phenyl)acetic acid

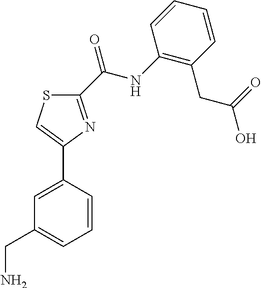 [SMILES: NCc1cccc(-c2csc(C(=O)Nc3ccccc3CC(=O)O)n2)c1]

The title compound (as the TFA salt) was synthesized as described in Example 17-B starting with methyl 2-(2-(4-bromothiazole-2-carboxamido)phenyl)acetate. ¹H NMR (TFA salt, 400 MHz, DMSO-d₆) δ ppm 12.75 (br. s., 1H) 10.48 (s, 1H) 8.46 (s, 1H) 8.07-8.41 (m, 5H) 7.71 (d, J=7.83 Hz, 1H) 7.58 (t, J=7.83 Hz, 1H) 7.51 (d, J=7.83 Hz, 1H) 7.31-7.43 (m, 2H) 7.18-7.31 (m, 1H) 4.13 (br. s., 2H) 3.74 (s, 2H). HRMS calcd. for C₁₉H₁₇N₃O₃S (M+H)⁺ 368.1069. found 368.1056.

### Example 27

### 2-(2-(2-(3-(Aminomethyl)phenyl)thiazole-4-carboxamido)phenyl)acetic acid

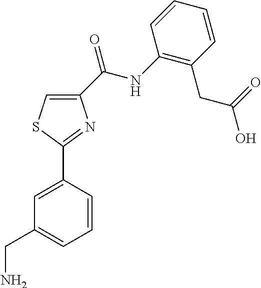 [SMILES: NCc1cccc(-c2nc(C(=O)Nc3ccccc3CC(=O)O)cs2)c1]

The title compound was synthesized as described in Example 26 starting with 2-bromothiazole-4-carboxylic acid (CAS #5198-88-9). ¹H NMR (TFA salt, 400 MHz, DMSO-d₆) δ ppm 12.70 (br. s., 1H) 10.22 (s, 1H) 8.52 (s, 1H) 8.06-8.35 (m, 5H) 7.80 (d, J=7.96 Hz, 1H) 7.56-7.69 (m, 2H) 7.28-7.44 (m, 2H) 7.21 (td, J=7.52, 1.26 Hz, 1H) 4.16 (br. s., 2H) 3.72 (s, 2H). HRMS calcd. for C₁₉H₁₇N₃O₃S (M+H)⁺ 368.1069. found 368.1060.

### Example 28

### Example 28-A

### 2-(2-(4-(3-(((tert-Butoxycarbonyl)amino)methyl)-5-fluorophenyl)thiazole-2-carboxamido)phenyl)acetic acid

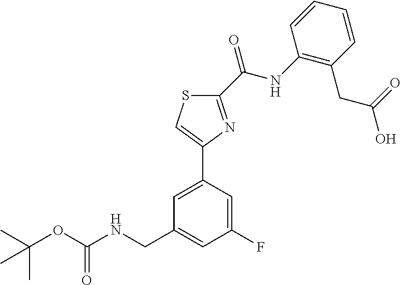 [SMILES: CC(C)(C)OC(=O)NCc1cc(F)cc(-c2csc(C(=O)Nc3ccccc3CC(=O)O)n2)c1]

In a 2-5 mL microwave vial with stir bar was added methyl 2-(2-(4-bromothiazole-2-carboxamido)phenyl)acetate (Example 26-A) (54 mg, 0.15 mmol) and tert-butyl 3-fluoro-5-(4,4,5,5-tetramethyl-1,3,2-dioxaborolan-2-yl)benzylcarbamate (Intermediate 24) (93 mg, 0.20 mmol) and MeCN (1.37 ml) and H₂O (0.152 ml). Then, 2M aq. K₃PO₄ (0.304 ml, 0.608 mmol) and PdCl₂(dppf).CH₂Cl₂ adduct (CAS #95464-05-4) (6.21 mg, 7.60 μmol) was added. The vial was sealed and the reaction mixture heated in a microwave at 110° C. for 1 hour. Additional tert-butyl 3-fluoro-5-(4,4,5,5-tetramethyl-1,3,2-dioxaborolan-2-yl)benzylcarbamate (50 mg, 0.11 mmol) and PdCl₂(dppf).CH₂Cl₂ adduct (CAS #95464-05-4) (6.21 mg, 7.60 μmol) were added and the reaction was heated in a microwave again at 110° C. for 1 hour. The organic layer was filtered and purified by preparative HPLC (Method B) to provide the title compound. MS (ESI−) m/z 484.5 (M−H).

### Example 28-B

### 2-(2-(4-(3-(aminomethyl)-5-fluorophenyl)thiazole-2-carboxamido)phenyl)acetic acid

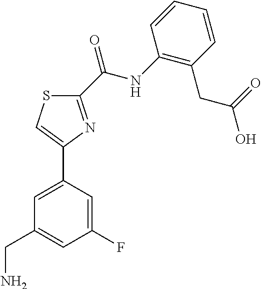 [SMILES: NCc1cc(F)cc(-c2csc(C(=O)Nc3ccccc3CC(=O)O)n2)c1]

To a suspension of 2-(2-(4-(3-(((tert-butoxycarbonyl)amino)methyl)-5-fluorophenyl)thiazole-2-carboxamido)phenyl)acetic acid (38 mg, 0.078 mmol) in diethyl ether (0.391 ml) was added HCl (2.0M in Ether, 0.783 ml, 1.565 mmol) and the reaction was stirred at 40° C. After stirring for 2 hours additional HCl (2.0M in Ether, 0.783 ml, 1.565 mmol) was added. After 2 more hours the reaction was concentrated and then purified by preparative HPLC (Method B) to provide the title compound. ¹H NMR (400 MHz, DMSO-d₆) δ ppm 12.34 (s, 1H) 8.80 (s, 1H) 8.59 (s, 1H) 7.89-7.94 (m, 1H) 7.86 (d, J=9.60 Hz, 1H) 7.18-7.31 (m, 3H) 7.03-7.13 (m, 1H) 4.10 (s, 2H) 3.50 (s, 2H). HRMS calcd. for C₁₉H₁₆FN₃O₃S (M+H)⁺ 386.0975. found 386.0965.

### Example 29

### 2-(2-(2-(3-(aminomethyl)-5-fluorophenyl)thiazole-4-carboxamido)phenyl)acetic acid

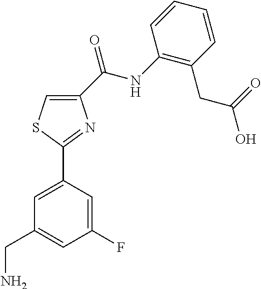 [SMILES: NCc1cc(F)cc(-c2nc(C(=O)Nc3ccccc3CC(=O)O)cs2)c1]

The title compound was synthesized as described in Example 28 starting with 2-bromothiazole-4-carboxylic acid (CAS #5198-88-9). ¹H NMR (400 MHz, DMSO-d₆) δ ppm 11.71 (s, 1H) 8.79 (s, 1H) 8.45 (s, 1H) 7.91-8.02 (m, 1H) 7.77 (d, J=8.72 Hz, 1H) 7.39 (d, J=9.60 Hz, 1H) 7.14-7.30 (m, 2H) 7.00-7.12 (m, 1H) 4.12 (s, 2H) 3.48 (s, 2H). HRMS calcd. for C₁₉H₁₆FN₃O₃S (M+H)⁺ 386.0975. found 386.0964.

### Example 30

### Example 30-A

### 2-Bromo-5-chlorothiazole-4-carboxylic acid

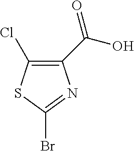

To a solution of methyl 2-bromo-5-chlorothiazole-4-carboxylate (CAS #1053655-63-2) (1.0 g, 3.9 mmol) in THF (23.4 ml) and MeOH (7.80 ml) and Water (7.80 ml) was added LiOH.H₂O (0.245 g, 5.85 mmol) and the reaction was stirred at room temperature. After 40 min. the reaction was concentrated, then dissolved in water. Conc. HCl was added until pH 1. The organics were extracted with EtOAc, combined, dried over MgSO₄, filtered and concentrated to provide the title compound. MS (ESI+) m/z 241.9, 244.0 (M+H).

### Example 30-B

### tert-Butyl 2-(2-(2-bromo-5-chlorothiazole-4-carboxamido)phenyl)acetate

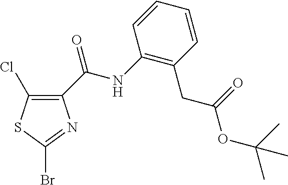 [SMILES: CC(C)(C)OC(=O)Cc1ccccc1NC(=O)c1nc(Br)sc1Cl]

The title compound was synthesized as described in Example 19-A starting with 2-bromo-5-chlorothiazole-4-carboxylic acid and tert-butyl 2-(2-aminophenyl)acetate (CAS #98911-34-3), and using (0-30% EtOAc:Heptanes) for the flash chromatography. MS (ESI−) m/z 429.2, 431.3 (M−H).

### Example 30-C

### tert-Butyl 2-(2-(2-(3-(((tert-butoxycarbonyl)amino)methyl)-5-fluorophenyl)-5-chlorothiazole-4-carboxamido)phenyl)acetate

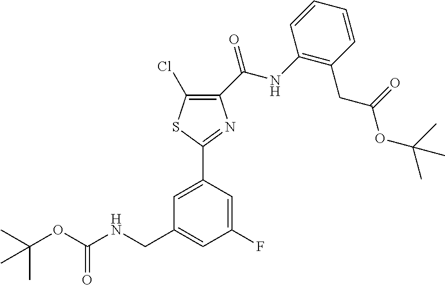 [SMILES: CC(C)(C)OC(=O)Cc1ccccc1NC(=O)c1nc(-c2cc(F)cc(CNC(=O)OC(C)(C)C)c2)sc1Cl]

To a solution of tert-butyl 2-(2-(2-bromo-5-chlorothiazole-4-carboxamido)phenyl)acetate (1.06 g, 2.46 mmol) and tert-butyl 3-fluoro-5-(4,4,5,5-tetramethyl-1,3,2-dioxaborolan-2-yl)benzylcarbamate (Intermediate 24) (1.03 g, 2.34 mmol) in DME (17.5 ml) under nitrogen was added Pd(PPh₃)₄ (0.540 g, 0.468 mmol) followed by a solution of K₂CO₃ (0.339 g, 2.46 mmol) in H₂O (5.85 ml), and the reaction was stirred under nitrogen at 80° C. After stirring overnight the reaction was diluted with water and EtOAc, extracted with EtOAc, dried over MgSO₄, filtered and concentrated. The crude product was purified by flash chromatography (0-50% EtOAc:Heptanes) to provide the title compound. MS (ESI−) m/z 574.5 (M−H).

### Example 30-D

### 2-(2-(2-(3-(aminomethyl)-5-fluorophenyl)-5-chlorothiazole-4-carboxamido)phenyl)acetic acid

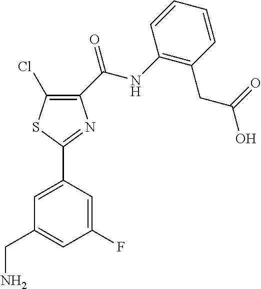 [SMILES: NCc1cc(F)cc(-c2nc(C(=O)Nc3ccccc3CC(=O)O)c(Cl)s2)c1]

The title compound was synthesized as described in Example 19-B starting with tert-butyl 2-(2-(2-(3-(((tert-butoxycarbonyl)amino)methyl)-5-fluorophenyl)-5-chlorothiazole-4-carboxamido)phenyl)acetate. ¹H NMR (400 MHz, METHANOL-d₄) δ ppm 8.52 (s, 1H) 7.89-8.00 (m, 1H) 7.63 (dt, J=8.84, 2.02 Hz, 1H) 7.23-7.37 (m, 3H) 7.14 (td, J=7.45, 1.26 Hz, 1H) 4.23 (s, 2H) 3.64 (s, 2H). HRMS calcd. for C₁₉H₁₅ClFN₃O₃S (M+H)⁺ 420.0585. found 420.0574.

### Example 31

### Example 31-A

### tert-Butyl 2-(2-(2-(3-(((tert-butoxycarbonyl)amino)methyl)-5-fluorophenyl)-5-(methylamino)thiazole-4-carboxamido)phenyl)acetate

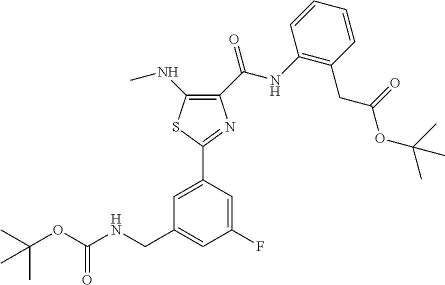 [SMILES: CNc1sc(-c2cc(F)cc(CNC(=O)OC(C)(C)C)c2)nc1C(=O)Nc1ccccc1CC(=O)OC(C)(C)C]

In a 2-5 mL microwave vessel was added tert-butyl 2-(2-(2-(3-(((tert-butoxycarbonyl)amino)methyl)-5-fluorophenyl)-5-chlorothiazole-4-carboxamido)phenyl)acetate (Example 30-C) (125 mg, 0.217 mmol), methylamine (33% in EtOH, 0.810 ml, 6.51 mmol), DIPEA (45.5 μl, 0.260 mmol) and DMSO (1.45 ml). The reaction was heated in the microwave at 180° C. for 30 minutes. The reaction was then diluted with EtOAc and water, and extracted with EtOAc. The combined organic layers were washed with water, then with brine, then dried with MgSO₄, filtered and concentrated to provide the title compound. MS (ESI−) m/z 569.6 (M−H).

### Example 31-B

### 2-(2-(2-(3-(aminomethyl)-5-fluorophenyl)-5-(methylamino)thiazole-4-carboxamido)phenyl)acetic acid

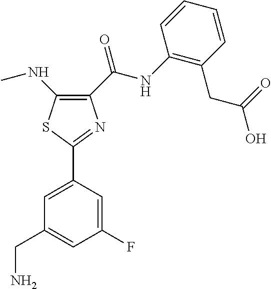 [SMILES: CNc1sc(-c2cc(F)cc(CN)c2)nc1C(=O)Nc1ccccc1CC(=O)O]

The title compound was synthesized as described in Example 20-B starting with tert-butyl 2-(2-(2-(3-(((tert-butoxycarbonyl)amino)methyl)-5-fluorophenyl)-5-(methylamino)thiazole-4-carboxamido)phenyl)acetate. ¹H NMR (600 MHz, METHANOL-d₄) δ ppm 8.36 (s, 1H) 7.93 (d, J=7.52 Hz, 1H) 7.35 (d, J=9.17 Hz, 1H) 7.21-7.29 (m, 2H) 7.13 (d, J=9.35 Hz, 1H) 7.08 (td, J=7.45, 1.15 Hz, 1H) 4.20 (s, 2H) 3.61 (s, 2H) 3.11 (s, 3H). HRMS calcd. for C₂₀H₁₉FN₄O₃S (M+H)⁺ 415.1240. found 415.1235.

### Example 32

### 2-(2-(2-(3-(aminomethyl)-5-fluorophenyl)-5-morpholinothiazole-4-carboxamido)phenyl)acetic acid

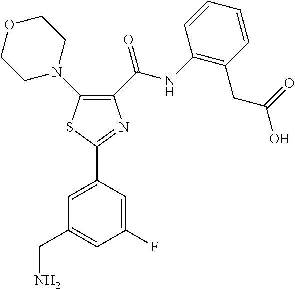 [SMILES: NCc1cc(F)cc(-c2nc(C(=O)Nc3ccccc3CC(=O)O)c(N3CCOCC3)s2)c1]

The title compound was synthesized as described in Example 31, using 7.5 eq. of morpholine in place of methanamine and heating at 130° C. for 60 minutes in Example 31-A. ¹H NMR (400 MHz, METHANOL-d₄) δ ppm 8.53 (s, 1H) 7.93 (dd, J=7.96, 0.88 Hz, 1H) 7.45 (dt, J=9.09, 1.89 Hz, 1H) 7.16-7.32 (m, 3H) 7.05-7.15 (m, 1H) 4.21 (s, 2H) 3.89 (dd, J=5.56, 3.79 Hz, 4H) 3.61 (s, 2H) 3.37-3.45 (m, 4H). HRMS calcd. for C₂₃H₂₃FN₄O₄S (M+H)⁺ 471.1502. found 471.1482.

### Example 33

### (S)-2-(2-(2-(3-(Aminomethyl)-5-fluorophenyl)-5-(((tetrahydrofuran-2-yl)methyl)amino)thiazole-4-carboxamido)phenyl)acetic acid

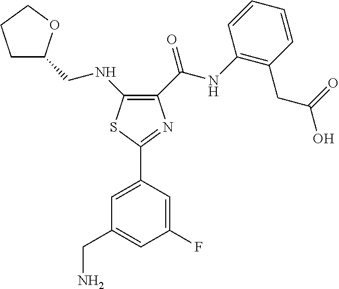 [SMILES: NCc1cc(F)cc(-c2nc(C(=O)Nc3ccccc3CC(=O)O)c(NC[C@@H]3CCCO3)s2)c1]

The title compound was synthesized as described in Example 31, using 10 eq. of (S)-(tetrahydrofuran-2-yl)methanamine (CAS #7175-81-7) in place of methanamine and heating at 130° C. for 60 minutes in Example 31-A. ¹H NMR (400 MHz, METHANOL-d₄) δ ppm 8.33 (s, 1H) 7.87-7.94 (m, 1H) 7.34 (d, J=9.35 Hz, 1H) 7.20-7.29 (m, 2H) 7.03-7.16 (m, 2H) 4.13-4.23 (m, 3H) 3.87-3.98 (m, 1H) 3.74-3.84 (m, 1H) 3.61 (s, 2H) 3.44-3.54 (m, 1H) 3.33-3.42 (m, 1H) 2.02-2.17 (m, 1H) 1.87-2.02 (m, 2H) 1.67-1.80 (m, 1H). HRMS calcd. for C₂₄H₂₅FN₄O₄S (M+H)⁺ 485.1659. found 485.1637.

### Example 34

### Example 34-A

### tert-Butyl 2-(2-(2-bromo-5-chlorothiazole-4-carboxamido)-4-methylphenyl)acetate

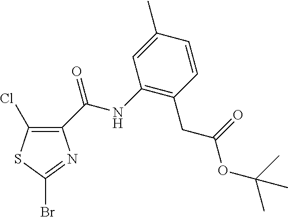 [SMILES: Cc1ccc(CC(=O)OC(C)(C)C)c(NC(=O)c2nc(Br)sc2Cl)c1]

The title compound was synthesized as described in Example 30-B starting with tert-butyl 2-(2-amino-4-methylphenyl)acetate (Intermediate 11). MS (ESI−) m/z 443.2, 445.2 (M−H).

### Example 34-B

### tert-Butyl 2-(2-(2-(3-(((tert-butoxycarbonyl)amino)methyl)-5-fluorophenyl)-5-chlorothiazole-4-carboxamido)-4-methylphenyl)acetate

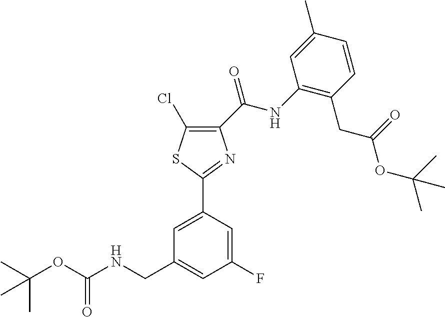 [SMILES: Cc1ccc(CC(=O)OC(C)(C)C)c(NC(=O)c2nc(-c3cc(F)cc(CNC(=O)OC(C)(C)C)c3)sc2Cl)c1]

To a solution of tert-butyl 2-(2-(2-bromo-5-chlorothiazole-4-carboxamido)-4-methylphenyl)acetate (390 mg, 0.700 mmol) and tert-butyl 3-fluoro-5-(4,4,5,5-tetramethyl-1,3,2-dioxaborolan-2-yl)benzylcarbamate (Intermediate 24) (246 mg, 0.700 mmol) in DME (7.00 ml) under nitrogen was added PdCl₂(dppf).CH₂Cl₂ adduct (CAS #95464-05-4) (57.2 mg, 0.070 mmol) followed by a 2M aqueous solution of K₂CO₃ (0.700 ml, 1.40 mmol), and the reaction was stirred under nitrogen at 80° C. After stirring overnight the reaction was diluted with water and saturated aqueous brine and EtOAc, the layers were separated and the aqueous layer was extracted with EtOAc, dried over MgSO₄, filtered and concentrated. The crude product was purified by flash chromatography (0-50% EtOAc:Heptanes) to provide the title compound. MS (ESI−) m/z 588.2 (M−H).

### Example 34-C

### 2-(2-(2-(3-(Aminomethyl)-5-fluorophenyl)-5-(methylamino)thiazole-4-carboxamido)-4-methylphenyl)acetic acid

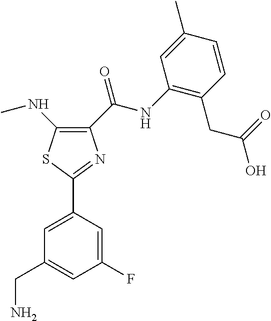 [SMILES: CNc1sc(-c2cc(F)cc(CN)c2)nc1C(=O)Nc1cc(C)ccc1CC(=O)O]

The title compound was synthesized as described in Example 31, starting with tert-butyl 2-(2-(2-(3-(((tert-butoxycarbonyl)amino)methyl)-5-fluorophenyl)-5-chlorothiazole-4-carboxamido)-4-methylphenyl)acetate and heating at 130° C. for 30 minutes in Example 31-A. ¹H NMR (400 MHz, METHANOL-d₄) δ ppm 8.35 (s, 1H) 7.76 (s, 1H) 7.34 (d, J=9.60 Hz, 1H) 7.07-7.19 (m, 2H) 6.90 (d, J=7.71 Hz, 1H) 4.19 (s, 2H) 3.56 (s, 2H) 3.11 (s, 3H) 2.35 (s, 3H). HRMS calcd. for C₂₁H₂₁FN₄O₃S (M+H)⁺ 429.1397. found 429.1383.

### Example 35

### Example 35-A

### tert-Butyl 2-(2-(2-(3-(((tert-butoxycarbonyl)amino)methyl)-5-fluorophenyl)-5-cyclopropylthiazole-4-carboxamido)phenyl)acetate

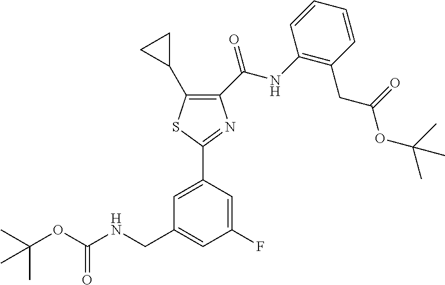 [SMILES: CC(C)(C)OC(=O)Cc1ccccc1NC(=O)c1nc(-c2cc(F)cc(CNC(=O)OC(C)(C)C)c2)sc1C1CC1]

In a microwave vial, to a solution of tert-butyl 2-(2-(2-(3-(((tert-butoxycarbonyl)amino)methyl)-5-fluorophenyl)-5-chlorothiazole-4-carboxamido)phenyl)acetate (Example 30-C) (120 mg, 0.208 mmol) and potassium cyclopropyltrifluoroborate (CAS #1065010-87-8) (61.6 mg, 0.417 mmol) in DME (1.56 ml) was added H₂O (0.52 ml) and K₂CO₃ (57.6 mg, 0.417 mmol) and finally Pd(PPh₃)₄ (48.1 mg, 0.042 mmol). The reaction was heated in the microwave at 120° C. for 1 hour. Additional potassium cyclopropyltrifluoroborate (61.6 mg, 0.417 mmol) and K₂CO₃ (57.6 mg, 0.417 mmol) and S-Phos palladacycle (CAS 1028206-58-7) (28.0 mg, 0.042 mmol) was added and the reaction was heated again at 120° C. for 1 hour. Additional S-Phos palladacycle (CAS 1028206-58-7) (28.0 mg, 0.042 mmol) was added and the reaction was stirred at 150° C. for 1 hour. The reaction was diluted with water and EtOAc, extracted with EtOAc, dried over MgSO₄, filtered and concentrated. The crude was purified by flash chromatography (0-50% EtOAc:Heptanes) to provide the title compound. MS (ESI−) m/z 580.5 (M−H).

### Example 35-B

### 2-(2-(2-(3-(Aminomethyl)-5-fluorophenyl)-5-cyclopropylthiazole-4-carboxamido)phenyl)acetic acid

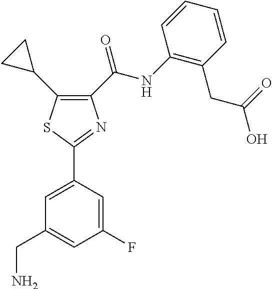 [SMILES: NCc1cc(F)cc(-c2nc(C(=O)Nc3ccccc3CC(=O)O)c(C3CC3)s2)c1]

The title compound was synthesized as described in Example 20-B starting with tert-butyl 2-(2-(2-(3-(((tert-butoxycarbonyl)amino)methyl)-5-fluorophenyl)-5-cyclopropylthiazole-4-carboxamido)phenyl)acetate. ¹H NMR (400 MHz, METHANOL-d₄) δ ppm 8.52 (s, 1H) 7.97 (d, J=7.83 Hz, 1H) 7.49 (d, J=9.22 Hz, 1H) 7.17-7.36 (m, 3H) 7.05-7.17 (m, 1H) 4.20 (s, 2H) 3.64 (s, 2H) 3.34-3.39 (m, 1H) 1.24-1.42 (m, 2H) 0.77-0.93 (m, 2H). HRMS calcd. for C₂₂H₂₀FN₃O₃S (M+H)⁺ 426.1288. found 426.1280.

### Example 36

### Example 36-A

### tert-Butyl 2-(2-(2-(3-(((tert-butoxycarbonyl)amino)methyl)-5-fluorophenyl)-5-vinylthiazole-4-carboxamido)phenyl)acetate

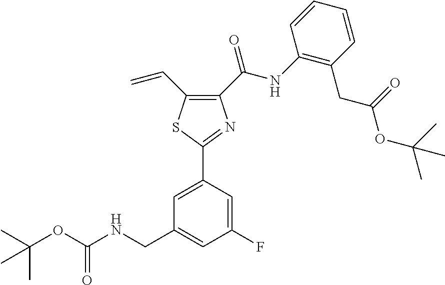 [SMILES: C=Cc1sc(-c2cc(F)cc(CNC(=O)OC(C)(C)C)c2)nc1C(=O)Nc1ccccc1CC(=O)OC(C)(C)C]

In a microwave vial, to a solution of tert-butyl 2-(2-(2-(3-(((tert-butoxycarbonyl)amino)methyl)-5-fluorophenyl)-5-chlorothiazole-4-carboxamido)phenyl)acetate (Example 30-C) (120 mg, 0.208 mmol) and vinylboronic anhydride pyridine complex (CAS #442850-89-7) (100 mg, 0.417 mmol) in DME (1.56 ml) was added H₂O (0.52 ml) and K₂CO₃ (57.6 mg, 0.417 mmol) and finally Pd(PPh₃)₄ (48.1 mg, 0.042 mmol), and the reaction was heated in the microwave at 120° C. for 1 hour. Additional vinylboronic anhydride pyridine complex (50 mg) and Pd(PPh₃)₄ (24 mg) and K₂CO₃ (29 mg) were added and the reaction was stirred again in the microwave at 120° C. for 45 minutes. The reaction was diluted with water and EtOAc, extracted with EtOAc, dried over MgSO₄, filtered and concentrated. The crude was purified by flash chromatography (0-50% EtOAc:Heptanes) to provide the title compound. MS (ESI−) m/z 566.5 (M−H).

### Example 36-B

### tert-Butyl 2-(2-(2-(3-(((tert-butoxycarbonyl)amino)methyl)-5-fluorophenyl)-5-ethylthiazole-4-carboxamido)phenyl)acetate

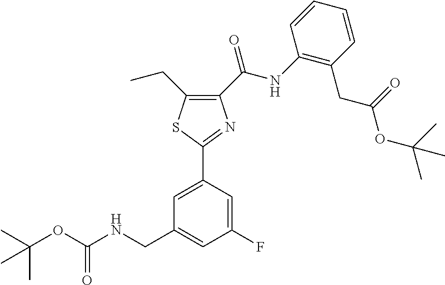 [SMILES: CCc1sc(-c2cc(F)cc(CNC(=O)OC(C)(C)C)c2)nc1C(=O)Nc1ccccc1CC(=O)OC(C)(C)C]

The title compound was synthesized as described in Intermediate 1-B (using EtOAc instead of EtOH as the solvent), starting with tert-butyl 2-(2-(2-(3-(((tert-butoxycarbonyl)amino)methyl)-5-fluorophenyl)-5-vinylthiazole-4-carboxamido)phenyl)acetate. MS (ESI−) m/z 568.6 (M−H).

### Example 36-C

### 2-(2-(2-(3-(Aminomethyl)-5-fluorophenyl)-5-ethylthiazole-4-carboxamido)phenyl)acetic acid

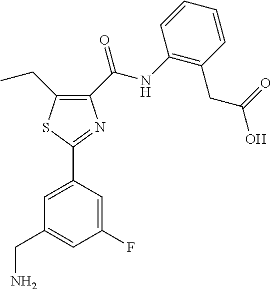 [SMILES: CCc1sc(-c2cc(F)cc(CN)c2)nc1C(=O)Nc1ccccc1CC(=O)O]

The title compound was synthesized as described in Example 20-B starting with tert-butyl 2-(2-(2-(3-(((tert-butoxycarbonyl)amino)methyl)-5-fluorophenyl)-5-ethylthiazole-4-carboxamido)phenyl)acetate. ¹H NMR (400 MHz, METHANOL-d₄) δ ppm 8.55 (s, 1H) 7.90-8.00 (m, 1H) 7.52-7.62 (m, 1H) 7.22-7.35 (m, 3H) 7.07-7.18 (m, 1H) 4.22 (s, 2H) 3.64 (s, 2H) 3.44 (q, J=7.49 Hz, 2H) 1.41 (t, J=7.52 Hz, 3H). HRMS calcd. for C₂₁H₂₀FN₃O₃S (M+H)⁺ 414.1288. found 414.1273.

### Example 37

### 2-(2-(3′-(Aminomethyl)-2′-fluoro-[1,1′-biphenyl]-3-ylcarboxamido)phenyl)acetic acid

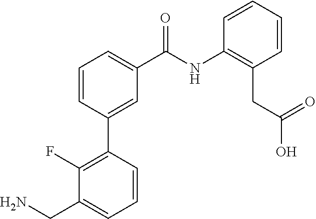 [SMILES: NCc1cccc(-c2cccc(C(=O)Nc3ccccc3CC(=O)O)c2)c1F]

A mixture of methyl 2-(2-(3-bromobenzamido)phenyl)acetate (Example 2-A) (0.15 g, 0.431 mmol), (3-(aminomethyl)-2-fluorophenyl)boronic acid hydrochloride (CAS #1072946-44-1) (0.109 g, 0.646 mmol), PdCl₂(dppf).CH₂Cl₂ adduct (CAS #95464-05-4) (0.018 g, 0.022 mmol), and 2M aq. K₃PO₄ (0.646 mL, 1.292 mmol) in CH₃CN (10 mL) was heated at 110° C. for 1 hr. The mixture was concentrated and the residue was purified by via HPLC (Method B) to provide the title compound. ¹H NMR (400 MHz, DMSO-d₆) δ ppm 13.82 (s, 1H) 8.23 (s, 1H) 8.09 (d, J=7.82 Hz, 1H) 7.93 (d, J=7.95 Hz, 1H) 7.61-7.71 (m, 1H) 7.50-7.60 (m, 1H) 7.43 (t, J=7.58 Hz, 2H) 7.16-7.23 (m, 1H) 7.04-7.15 (m, 2H) 6.87-6.99 (m, 1H) 3.80 (s, 2H) 3.30 (s, 2H). HRMS calcd. for C₂₂H₁₉FN₂O₃(M+H)⁺ 379.1451. found 379.1458.

### Example 38

### 2-(2-(2-(3-(Aminomethyl)phenyl)isonicotinamido)phenyl)acetic acid

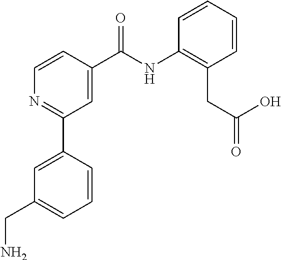 [SMILES: NCc1cccc(-c2cc(C(=O)Nc3ccccc3CC(=O)O)ccn2)c1]

A microwave vial equipped with a magnetic stirring bar was charged with 3-aminomethylphenyl boronic acid hydrochloride (CAS #146285-80-5) (79 mg, 0.423 mmol), methyl 2-(2-(2-chloroisonicotinamido)phenyl)acetate (Intermediate 42) (86 mg, 0.282 mmol) and PdCl₂(dppf).CH₂Cl₂ adduct (CAS #95464-05-4) (11.52 mg, 0.014 mmol). Acetonitrile (2.5 mL), water (0.250 mL) and 2M aq. K₃PO₄ (0.564 mL, 1.129 mmol) were added, the microwave vial was flushed with nitrogen and sealed. The resulting suspension was heated in the microwave at 110° C. for 60 minutes. The organic phase was separated, filtered and concentrated. The residue was purified by preparative HPLC (Method B) affording the title compound after lyophilization. ¹H NMR (400 MHz, METHANOL-d₄) δ ppm 8.84 (br. d, J=1.0 Hz, 1H), 8.60 (br. s, 1H), 8.18 (s, 2H), 8.03-7.96 (m, 2H), 7.59 (t, J=1.0 Hz, 1H), 7.54-7.50 (m, 1H), 7.36-7.27 (m, 2H), 7.15 (dt, J=1.1, 7.5 Hz, 1H), 4.17 (s, 2H), 3.64 (s, 2H). HRMS calcd. for C₂₁H₁₉N₃O₃ (M+H)⁺ 362.1505. found 362.1484.

### Example 39

### 2-(2-(4-(3-(Aminomethyl)phenyl)picolinamido)phenyl)acetic acid

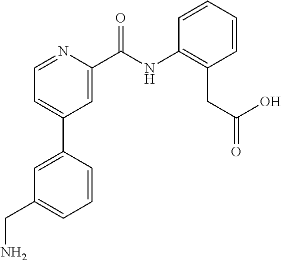 [SMILES: NCc1cccc(-c2ccnc(C(=O)Nc3ccccc3CC(=O)O)c2)c1]

The title compound was prepared from methyl 2-(2-(4-chloropicolinamido)phenyl)acetate (Intermediate 43) as described in Example 38. ¹H NMR (400 MHz, METHANOL-d₄) δ ppm 8.77 (d, J=4.8 Hz, 1H), 8.49 (br. s, 1H), 7.95 (dd, J=0.9, 8.0 Hz, 1H), 7.87 (dd, J=1.8, 5.1 Hz, 1H), 7.85 (br. s, 1H), 7.74 (d, J=7.3 Hz, 1H), 7.52 (td, J=7.5, 14.8 Hz, 2H), 7.34-7.25 (m, 2H), 7.15 (dt, J=1.3, 7.6 Hz, 1H), 3.60 (s, 2H), 3.30 (s, 2H). HRMS calcd. for C₂₁H₁₉N₃O₃ (M+H)⁺ 362.1505. found 362.1496.

### Example 40

### 2-(2-(5-(3-(Aminomethyl)phenyl)nicotinamido)phenyl)acetic acid

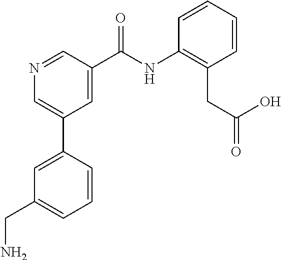 [SMILES: NCc1cccc(-c2cncc(C(=O)Nc3ccccc3CC(=O)O)c2)c1]

The title compound was prepared from methyl 2-(2-(5-bromonicotinamido)phenyl)acetate (Intermediate 44) as described in Example 38. ¹H NMR (400 MHz, METHANOL-d₄) δ ppm 9.21 (d, J=2.0 Hz, 1H), 9.02 (d, J=2.3 Hz, 1H), 8.83 (t, J=2.0 Hz, 1H), 7.99-7.98 (m, 1H), 7.87-7.83 (m, 1H), 7.75 (d, J=7.6 Hz, 1H), 7.54 (t, J=1.0 Hz, 1H), 7.49-7.44 (m, 1H), 7.35-7.24 (m, 3H), 7.14 (q, J=14.9 Hz, 1H), 4.01 (s, 2H), 3.63 (br. s, 2H). HRMS calcd. for C₂₁H₁₉N₃O₃ (M+H)⁺ 362.1505. found 361.1494.

### Example 41

### 2-(2-(6-(3-(Aminomethyl)phenyl)-4-chloropicolinamido)phenyl)acetic acid

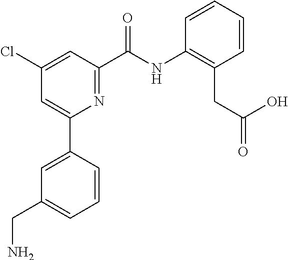 [SMILES: NCc1cccc(-c2cc(Cl)cc(C(=O)Nc3ccccc3CC(=O)O)n2)c1]

A DCM (0.5 mL) solution of tert-butyl 2-(2-(6-(3-(((tert-butoxycarbonyl)amino)methyl)phenyl)-4-chloropicolinamido)phenyl)acetate (Intermediate 49-B) (51 mg, 0.092 mmol) was treated with TFA (0.5 mL) at room temperature. After 1 hr, acetonitrile (1 mL) was added the solution was concentrated. Purification of the residue using preparative HPLC (Method A) afforded the title compound as a TFA salt. ¹H NMR (TFA salt, 400 MHz, METHANOL-d₄) δ ppm 8.53 (s, 1H), 8.32-8.25 (m, 2H), 8.19 (d, J=1.8 Hz, 1H), 7.94 (d, J=8.3 Hz, 1H), 7.67-7.55 (m, 2H), 7.42-7.34 (m, 2H), 7.24 (dt, J=1.3, 7.6 Hz, 1H), 4.28 (s, 2H), 3.80 (s, 2H). HRMS calcd. for C₂₁H₁₈ClN₃O₃(M+H)⁺ 396.1115. found 395.1098.

### Example 42

### 2-(2-(4,6-bis(3-(Aminomethyl)phenyl)picolinamido)phenyl)acetic acid

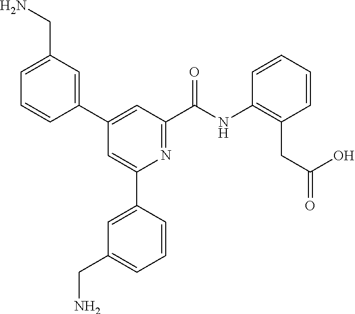 [SMILES: NCc1cccc(-c2cc(C(=O)Nc3ccccc3CC(=O)O)nc(-c3cccc(CN)c3)c2)c1]

The title compound was prepared from tert-butyl 2-(2-(6-(3-(((tert-butoxycarbonyl)amino)methyl)phenyl)-4-chloropicolinamido)phenyl)acetate (Intermediate 49-B) and (3-(((tert-butoxycarbonyl)amino)methyl)phenyl)boronic acid using the method described in Intermediate 49-B. The crude residue was treated with 1/1 TFA/DCM (2 mL) and then purified by preparative HPLC (Method A) affording the title compound as TFA salt. ¹H NMR (TFA salt, 400 MHz, DMSO-d₆) δ ppm 11.56-11.23 (m, 1H), 8.89 (br. s., 1H), 8.58 (d, J=1.4 Hz, 1H), 8.48 (d, J=1.4 Hz, 1H), 8.41 (d, J=7.3 Hz, 1H), 9.03-8.21 (m, 5H), 8.16 (s, 1H), 8.13-8.05 (m, 1H), 7.92 (d, J=7.8 Hz, 1H), 7.70-7.54 (m, 4H), 7.38-7.31 (m, 2H), 7.18 (dt, J=1.0, 7.3 Hz, 1H), 7.48-6.89 (m, 2H), 4.21 (s, 2H), 4.18 (s, 2H), 3.65 (s, 2H). HRMS calcd. for C₂₈H₂₆N₄O₃ (M+H)⁺ 467.2083. found 467.2192.

### Example 43

### 2-(2-(2-(3-(Aminomethyl)phenyl)-1H-imidazole-4-carboxamido)phenyl)acetic acid

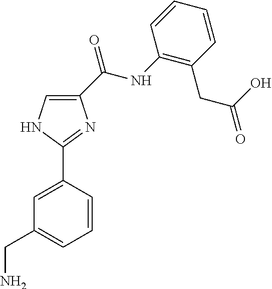 [SMILES: NCc1cccc(-c2nc(C(=O)Nc3ccccc3CC(=O)O)c[nH]2)c1]

The title compound was prepared from tert-butyl 2-(2-(2-bromo-1-((2-(trimethylsilyl)ethoxy)methyl)-1H-imidazole-5-carboxamido)phenyl)acetate (Intermediate 47-D) and 3-aminomethylphenyl boronic acid HCl (CAS #146285-80-5) using the method described in Example 38. The crude residue was treated with 1/1 TFA/DCM (2 mL) and then purified by preparative HPLC (Method A) affording the title compound as TFA salt. ¹H NMR (TFA salt, 400 MHz, METHANOL-d₄) δ ppm 8.18 (br. s, 1H), 8.11 (br. s, 1H), 8.05 (td, J=2.0, 6.7 Hz, 1H), 7.80-7.71 (m, 2H), 7.63 (d, J=7.3 Hz, 1H), 7.44-7.33 (m, 2H), 7.33-7.24 (m, 1H), 4.28 (s, 2H), 3.76 (s, 2H). HRMS calcd. for C₁₉H₁₈N₄O₃ (M+H)⁺ 351.1457. found 351.1548.

### Example 44

### 2-(2-(6-(3-(Aminomethyl)phenyl)-4-morpholinopicolinamido)phenyl)acetic acid

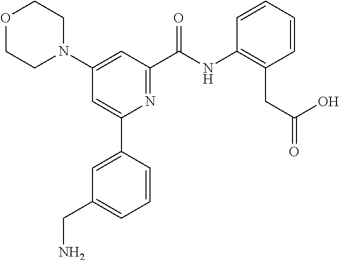 [SMILES: NCc1cccc(-c2cc(N3CCOCC3)cc(C(=O)Nc3ccccc3CC(=O)O)n2)c1]

A DMSO (0.2 mL) solution of tert-butyl 2-(2-(6-(3-(((tert-butoxycarbonyl)amino)methyl)phenyl)-4-chloropicolinamido)phenyl)acetate (Intermediate 49-B) (50 mg, 0.091 mmol), morpholine (0.024 mL, 0.272 mmol), and DIEA (0.019 mL, 0.109 mmol) was microwaved at 130° C. for 90 minutes. The reaction was partitioned between ethyl acetate and water, washed with water, brine, dried (sodium sulfate), filtered, and concentrated. The residue was treated with 1/1 TFA/DCM (2 mL) for 3 hours, concentrated and purified by preparative HPLC (Method A) to provide the title compound as TFA salt. ¹H NMR (TFA salt, 400 MHz, METHANOL-d₄) δ ppm 8.11 (s, 1H), 7.94 (d, J=7.5 Hz, 1H), 7.84 (d, J=2.1 Hz, 1H), 7.78-7.64 (m, 3H), 7.50 (d, J=2.3 Hz, 1H), 7.44-7.36 (m, 2H), 7.32 (dt, J=1.3, 7.5 Hz, 1H), 4.28 (s, 2H), 3.90 (s, 8H), 3.79 (s, 2H). HRMS calcd. for C₂₅H₂₆N₄O₄ (M+H)⁺ 447.2033. found 447.2012.

### Example 45

### (S)-2-(2-(6-(3-(Aminomethyl)phenyl)-4-(((tetrahydrofuran-2-yl)methyl)amino)picolinamido)phenyl)acetic acid

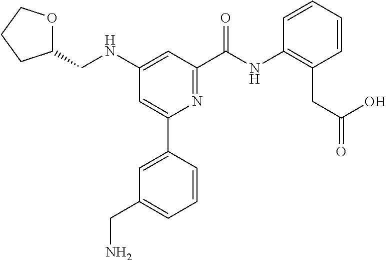

The title compound was prepared from tert-butyl 2-(2-(6-(3-(((tert-butoxycarbonyl)amino)methyl)phenyl)-4-chloropicolinamido)phenyl)acetate (Intermediate 49-B) and (S)-(tetrahydrofuran-2-yl)methanamine (CAS #7175-81-7) (10 equiv, 3 h, 130° C.) using the method described for Example 44. ¹H NMR (TFA salt, 400 MHz, METHANOL-d₄) δ ppm 8.44 (br. s, 1H), 8.10 (br. d, J=7.8 Hz, 1H), 7.93 (d, J=7.8 Hz, 1H), 7.56 (t, J=7.8 Hz, 1H), 7.50 (d, J=8.5 Hz, 1H), 7.45 (d, J=2.1 Hz, 1H), 7.40-7.32 (m, 2H), 7.28 (d, J=2.1 Hz, 1H), 7.21 (dt, J=1.1, 7.4 Hz, 1H), 4.29-4.23 (m, 2H), 4.16 (dq, J=4.4, 6.8 Hz, 1H), 3.91 (td, J=6.8, 8.1 Hz, 1H), 3.82-3.74 (m, 3H), 3.49-3.34 (m, 2H), 2.16-2.05 (m, 1H), 2.05-1.89 (m, 2H), 1.80-1.65 (m, 1H). HRMS calcd. for C₂₆H₂₈N₄O₄ (M+H)⁺ 461.2189. found 461.2169.

### Example 46

### 2-(2-(6-(3-(aminomethyl)phenyl)-4-(methylamino)picolinamido)phenyl)acetic acid

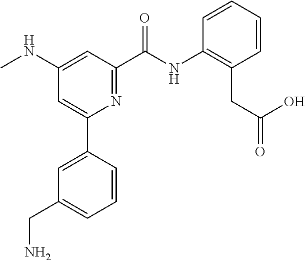 [SMILES: CNc1cc(C(=O)Nc2ccccc2CC(=O)O)nc(-c2cccc(CN)c2)c1]

The title compound was prepared as in Example 45 using methylamine (2 M in THF, 20 equiv, 6 h, 180° C.) using preparative HPLC (Method B). ¹H NMR (400 MHz, DMSO-d₆) δ ppm 11.94 (s, 1H), 9.22 (s, 1H), 9.69-8.83 (m, 3H), 7.98 (d, J=7.5 Hz, 1H), 7.82 (d, J=7.2 Hz, 1H), 7.49-7.42 (m, 1H), 7.42-7.36 (m, 1H), 7.29-7.16 (m, 4H), 7.10-7.02 (m, 1H), 6.98 (d, J=4.9 Hz, 1H), 4.07 (s, 2H), 3.38 (s, 2H), 2.88 (d, J=4.8 Hz, 3H). HRMS calcd. for C₂₂H₂₂N₄O₃ (M+H)⁺ 391.1770. found 391.1753.

### Example 47

### 2-(2-(4-(3-(Aminomethyl)phenyl)-1H-imidazole-2-carboxamido)phenyl)acetic acid

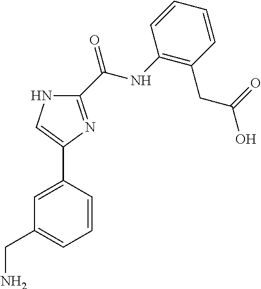 [SMILES: NCc1cccc(-c2c[nH]c(C(=O)Nc3ccccc3CC(=O)O)n2)c1]

The title compound was prepared from tert-butyl 2-(2-(5-bromo-1-((2-(trimethylsilyl)ethoxy)methyl)-1H-imidazole-2-carboxamido)phenyl)acetate (Intermediate 46-D) and 3-aminomethylphenyl boronic acid hydrochloride salt (CAS #146285-80-5) using the method described in Example 38. The crude residue was treated with 1/1 TFA/DCM (2 mL) and then purified by preparative HPLC (Method B) to afford the title compound. ¹H NMR (400 MHz, METHANOL-d₄) δ ppm 8.17 (br. s, 1H), 8.11 (br. s, 1H), 8.05 (td, J=2.0, 6.7 Hz, 1H), 7.80-7.71 (m, 2H), 7.63 (d, J=7.3 Hz, 1H), 7.44-7.33 (m, 2H), 7.29 (dt, J=1.1, 7.5 Hz, 1H), 4.28 (s, 2H), 3.76 (s, 2H). HRMS calcd. for C₁₉H₁₈N₄O₃ (M+H)⁺ 351.1457. found 351.1449.

### Example 48

### 2-(2-(3′-(Aminomethyl)-[1,1′-biphenyl]-3-ylsulfonamido)phenyl)acetic acid

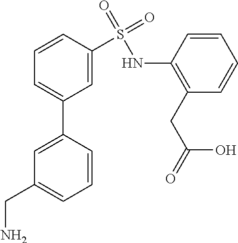 [SMILES: NCc1cccc(-c2cccc(S(=O)(=O)Nc3ccccc3CC(=O)O)c2)c1]

The title compound was prepared from tert-butyl 2-(2-(3-bromophenylsulfonamido)phenyl)acetate (Intermediate 48) as in Example 47. ¹H NMR (400 MHz, DMSO-d₆) δ ppm 8.83 (br. s, 2H), 8.47 (s, 1H), 8.00 (s, 1H), 7.92 (d, J=7.8 Hz, 1H), 7.89-7.81 (m, 1H), 7.72 (d, J=8.1 Hz, 1H), 7.62 (t, J=7.8 Hz, 1H), 7.53-7.44 (m, 1H), 7.39 (d, J=7.7 Hz, 1H), 7.23 (d, J=8.2 Hz, 1H), 7.04 (dd, J=1.3, 7.5 Hz, 1H), 6.99 (dt, J=1.6, 7.8 Hz, 1H), 6.82 (dt, J=0.9, 7.4 Hz, 1H), 4.09 (s, 2H), 3.36 (s, 2H). HRMS calcd. for C₂₁H₂₀N₂O₄S (M+H)⁺ 397.1222. found 397.1217.

### Example 49

### 2-(2-(3′-(aminomethyl)-5′-fluoro-[1,1′-biphenyl]-3-ylsulfonamido)phenyl)acetic acid

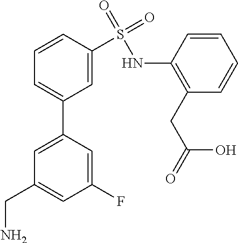 [SMILES: NCc1cc(F)cc(-c2cccc(S(=O)(=O)Nc3ccccc3CC(=O)O)c2)c1]

The title compound was prepared from tert-butyl 2-(2-(3-bromophenylsulfonamido)phenyl)acetate (Intermediate 48) and tert-butyl 3-fluoro-5-(4,4,5,5-tetramethyl-1,3,2-dioxaborolan-2-yl)benzylcarbamate (Intermediate 24) using the method described in Example 38. The crude residue was treated with 1/1 TFA/DCM (2 mL) and then purified by preparative HPLC (Method B) to afford the title compound. ¹H NMR (400 MHz, DMSO-d₆) δ ppm 8.29 (br. s, 1H), 8.02-7.94 (m, 1H), 7.84 (br. d, J=7.7 Hz, 1H), 7.78 (s, 1H), 7.70-7.63 (m, 1H), 7.59 (td, J=1.8, 10.4 Hz, 1H), 7.32 (br. d, J=9.2 Hz, 1H), 7.13 (d, J=7.3 Hz, 1H), 7.03 (d, J=4.3 Hz, 2H), 7.00-6.92 (m, 1H), 4.12 (s, 2H), 3.47 (s, 2H). HRMS calcd. for C₂₁H₁₉FN₂O₄S (M+H)⁺ 415.1128. found 415.1115.

### Example 50

### 2-(2-(6-(3-(aminomethyl)phenyl)-4-(cyclopropylamino)picolinamido)phenyl)acetic acid

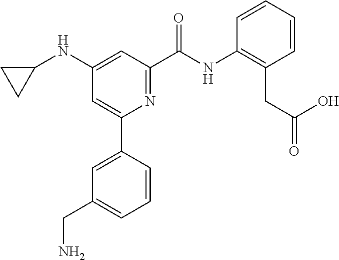 [SMILES: NCc1cccc(-c2cc(NC3CC3)cc(C(=O)Nc3ccccc3CC(=O)O)n2)c1]

The title compound was prepared from tert-butyl 2-(2-(6-(3-(((tert-butoxycarbonyl)amino)methyl)phenyl)-4-chloropicolinamido)phenyl)acetate (Intermediate 49-B) and cyclopropylamine (80 equiv, 8 h, 180° C.) using the method described in Example 45. ¹H NMR (400 MHz, DMSO-d₆) δ ppm 11.94 (s, 1H), 9.26-9.17 (m, 1H), 9.21 (br. s, 2H), 7.90 (d, J=7.8 Hz, 1H), 7.82 (d, J=7.1 Hz, 1H), 7.49-7.43 (m, 1H), 7.43-7.30 (m, 4H), 7.26-7.16 (m, 2H), 7.06 (dt, J=1.0, 7.3 Hz, 1H), 4.07 (s, 2H), 3.38 (s, 2H), 2.64-2.56 (m, 1H), 0.88-0.80 (m, 2H), 0.53-0.47 (m, 2H). HRMS calcd. for C₂₄H₂₄N₄O₃ (M+H)⁺417.1927. found 417.1913.

### Example 51

### 2-(2-(6-(3-(Aminomethyl)phenyl)-4-(4-phenylpiperazin-1-yl)picolinamido)phenyl)acetic acid

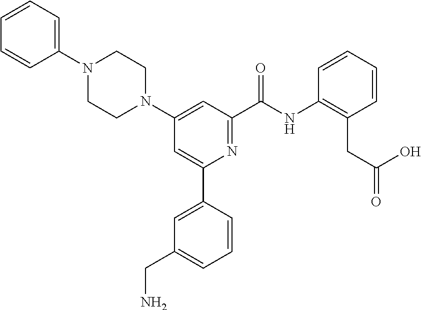 [SMILES: NCc1cccc(-c2cc(N3CCN(c4ccccc4)CC3)cc(C(=O)Nc3ccccc3CC(=O)O)n2)c1]

The title compound was prepared as in Example 50 using 1-phenylpiperazine (2 equiv, 2 h, 130° C.). ¹H NMR (400 MHz, DMSO-d₆) δ ppm 12.05 (s, 1H), 9.28-9.24 (m, 1H), 9.27 (br. s, 2H), 8.20 (d, J=7.7 Hz, 1H), 7.83 (d, J=7.5 Hz, 1H), 7.71 (d, J=2.3 Hz, 1H), 7.58 (d, J=2.1 Hz, 1H), 7.47 (t, J=8.0 Hz, 1H), 7.41 (d, J=8.0 Hz, 1H), 7.26 (t, J=8.5 Hz, 2H), 7.23-7.17 (m, 1H), 7.07 (dt, J=1.1, 7.4 Hz, 1H), 7.02 (d, J=8.0 Hz, 1H), 6.83 (t, J=7.3 Hz, 1H), 4.08 (s, 2H), 3.79-3.65 (m, 4H), 3.39 (s, 2H), 3.34 (s, 2H). HRMS calcd. for C₃₁H₃₁N₅O₃ (M+H)⁺ 522.2505. found 522.2488.

### Example 52

### 2-(2-(6-(3-(Aminomethyl)phenyl)-4-phenylpicolinamido)phenyl)acetic acid

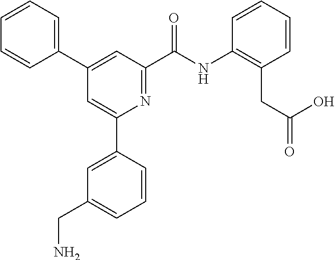 [SMILES: NCc1cccc(-c2cc(-c3ccccc3)cc(C(=O)Nc3ccccc3CC(=O)O)n2)c1]

A microwave vial was charged with tert-butyl 2-(2-(6-(3-(((tert-butoxycarbonyl)amino)methyl)phenyl)-4-chloropicolinamido)phenyl)acetate (Intermediate 49-B) (105 mg, 0.19 mmol), Pd(PPh₃)₄ (11 mg, 0.05 mmol), phenylboronic acid (32 mg, 0.266 mmol), cesium carbonate (124 mg, 0.38 mmol), followed by dioxane (1 mL) and water (0.2 mL). The microwave vial was heated at 110° C. for 1 hr, concentrated and treated with 1/1 TFA/DCM (2 mL). The mixture was purified using preparative HPLC (Method B) to give the title compound. ¹H NMR (400 MHz, DMSO-d₆) δ ppm 12.29 (s, 1H), 9.37 (s, 1H), 9.25 (br. s, 2H), 8.58 (d, J=1.5 Hz, 1H), 8.39-8.32 (m, 2H), 8.08-7.98 (m, 2H), 7.87 (d, J=8.8 Hz, 1H), 7.66-7.44 (m, 6H), 7.30-7.19 (m, 2H), 7.10 (dt, J=1.1, 7.5 Hz, 1H), 4.12 (s, 2H), 3.43 (s, 2H). HRMS calcd. for C₂₇H₂₃N₃O₃ (M+H)⁺ 438.1818. found 438.1805.

### Example 53

### 2-(2-(6-(3-(Aminomethyl)phenyl)-4-(benzylamino)picolinamido)phenyl)acetic acid

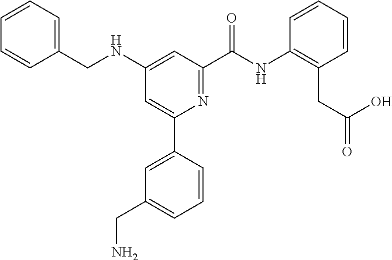 [SMILES: NCc1cccc(-c2cc(NCc3ccccc3)cc(C(=O)Nc3ccccc3CC(=O)O)n2)c1]

A vial was charged with tert-butyl 2-(2-(6-(3-(((tert-butoxycarbonyl)amino)methyl)phenyl)-4-chloropicolinamido)phenyl)acetate (Intermediate 49-B) (78 mg, 0.141 mmol), BrettPhos palladacycle (CAS #1148148-01-9) (2.82 mg, 3.53 μmol), benzylamine (0.019 mL, 0.170 mmol) and sodium tert-butoxide (16.29 mg, 0.170 mmol). Dioxane (1 mL) was added, the head space was purged with nitrogen. The sealed vial was heated overnight at 50° C. The crude reaction mixture was filtered, concentrated, then treated with 1/1 TFA/DCM (2 mL) for two hours. The residue was purified by preparative HPLC (Method B) to afford the title compound. ¹H NMR (400 MHz, DMSO-d₆) δ ppm 11.92 (s, 1H), 9.27 (br. s, 2H), 9.19 (s, 1H), 7.87 (d, J=7.1 Hz, 1H), 7.79 (d, J=8.0 Hz, 1H), 7.56 (t, J=6.4 Hz, 1H), 7.47-7.29 (m, 9H), 7.29-7.15 (m, 3H), 7.05 (t, J=7.6 Hz, 1H), 4.53 (d, J=5.7 Hz, 2H), 4.06 (s, 2H), 3.37 (s, 2H). HRMS calcd. for C₂₈H₂₆N₄O₃ (M+H)⁺ 467.2083. found 467.2064.

### Example 54

### 2-(2-(6-(3-(Aminomethyl)phenyl)-4-((2,2,2-trifluoroethyl)amino)picolinamido)phenyl)acetic acid

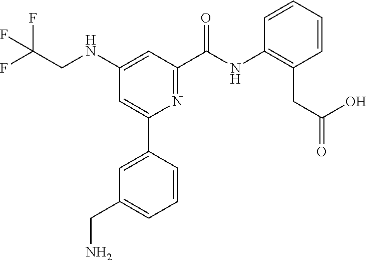 [SMILES: NCc1cccc(-c2cc(NCC(F)(F)F)cc(C(=O)Nc3ccccc3CC(=O)O)n2)c1]

The title compound was prepared as in Example 53 using trifluoroethylamine. ¹H NMR (400 MHz, DMSO-d₆) δ ppm 11.97 (br. s, 1H), 9.22 (s, 1H), 9.39-9.12 (m, 2H), 8.04 (d, J=8.0 Hz, 1H), 7.82 (d, J=7.6 Hz, 1H), 7.56 (d, J=1.9 Hz, 1H), 7.52-7.44 (m, 3H), 7.43-7.38 (m, J=8.1 Hz, 1H), 7.27-7.17 (m, 2H), 7.07 (dt, J=1.4, 7.4 Hz, 1H), 4.35-4.21 (m, 2H), 4.07 (br. s, 2H), 3.39 (s, 2H). HRMS calcd. for C₂₃H₂₁F₃N₄O₃(M+H)⁺ 459.1644. found 459.1643.

### Example 55

### 2-(2-(6-(3-(Aminomethyl)phenyl)-4-hydroxypicolinamido)phenyl)acetic acid

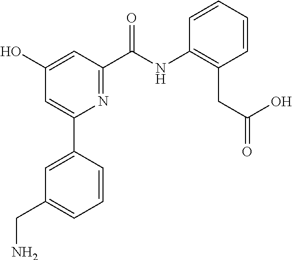 [SMILES: NCc1cccc(-c2cc(O)cc(C(=O)Nc3ccccc3CC(=O)O)n2)c1]

A toluene suspension of tert-butyl 2-(2-(6-(3-(((tert-butoxycarbonyl)amino)methyl)phenyl)-4-chloropicolinamido)phenyl)acetate (Intermediate 49-B) (100 mg, 0.181 mmol), KOH (51 mg, 0.91 mmol), 2M aq. K₃PO₄ (77 mg, 0.362 mmol), Pd(OAc)₂ (CAS #3375-31-3) (2.03 mg, 9.06 μmol) and t-Bu X-Phos (CAS #857356-94-6) (8.71 mg, 0.018 mmol) was heated in a microwave at 110° C. for 1 hr. The crude reaction was filtered, treated with TFA/DCM (2 mL) and then purified by preparative HPLC (Method B) to afford the title compound. ¹H NMR (400 MHz, DMSO-d₆) δ ppm 11.99 (br. s, 1H), 11.16 (br. s, 1H), 9.20 (s, 1H), 9.18 (br. s, 2H), 8.00 (d, J=7.5 Hz, 1H), 7.83 (dd, J=0.8, 7.9 Hz, 1H), 7.58 (d, J=2.0 Hz, 1H), 7.52 (d, J=2.1 Hz, 1H), 7.51-7.37 (m, 2H), 7.30-7.16 (m, 2H), 7.08 (dt, J=1.1, 7.4 Hz, 1H), 4.09 (s, 2H), 3.42 (s, 2H). HRMS calcd. for C₂₁H₁₉N₃O₄ (M+H)⁺ 378.1454. found 378.1447.

### Example 56

### Example 56-A

### 2-(2-(6-(3-(Aminomethyl)phenyl)-3-chloropicolinamido)phenyl)acetic acid

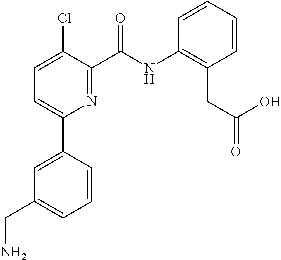 [SMILES: NCc1cccc(-c2ccc(Cl)c(C(=O)Nc3ccccc3CC(=O)O)n2)c1]

The title compound was prepared from tert-butyl 2-(2-(3,6-dichloropicolinamido)phenyl)acetate (Intermediate 45) and (3-(((tert-butoxycarbonyl)amino)methyl)phenyl)boronic acid using the method described in Intermediate 49-B. ¹H NMR (400 MHz, DMSO-d₆) δ ppm 12.03 (s, 1H), 9.27 (br. s, 1H), 9.19 (s, 1H), 8.29 (d, J=8.6 Hz, 1H), 8.17 (d, J=8.5 Hz, 1H), 8.10 (d, J=7.6 Hz, 1H), 7.81 (d, J=8.5 Hz, 1H), 7.55-7.43 (m, 1H), 7.29-7.17 (m, 1H), 7.09 (dt, J=1.1, 7.5 Hz, 1H), 4.13-4.05 (m, 2H), 3.39 (s, 2H). HRMS calcd. for C₂₁H₁₈N₃O₄ (M+H)⁺ 396.1115. found 396.1113.

### Example 56-B

### 2-(2-(3,6-bis(3-(Aminomethyl)phenyl)picolinamido)phenyl)acetic acid

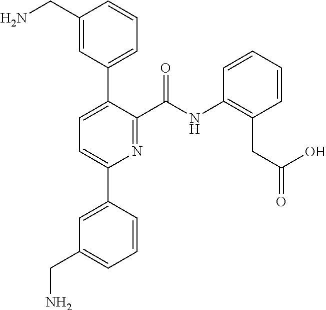 [SMILES: NCc1cccc(-c2ccc(-c3cccc(CN)c3)c(C(=O)Nc3ccccc3CC(=O)O)n2)c1]

The title compound was obtained as a by-product of the reaction in Example 56-A. ¹H NMR (400 MHz, DMSO-d₆) δ ppm 11.99 (br. s, 1H), 9.23 (br. s, 1H), 8.30 (d, J=8.3 Hz, 1H), 8.13 (d, J=7.6 Hz, 1H), 7.90 (d, J=8.1 Hz, 1H), 7.70 (dd, J=0.8, 8.0 Hz, 1H), 7.52 (t, J=7.7 Hz, 1H), 7.46 (d, J=7.7 Hz, 1H), 7.39 (s, 1H), 7.36-7.28 (m, 3H), 7.28-7.21 (m, 1H), 7.21-7.16 (m, 1H), 7.16-7.12 (m, 1H), 7.03 (dt, J=1.0, 7.5 Hz, 1H), 4.10 (s, 2H), 3.81 (s, 2H), 3.41 (s, 2H). HRMS calcd. for C₂₈H₂₆N₄O₃ (M+H)⁺ 467.2083. found 467.2046.

### Example 57

### 2-(2-(6-(3-(Aminomethyl)phenyl)picolinamido)phenyl)acetic acid

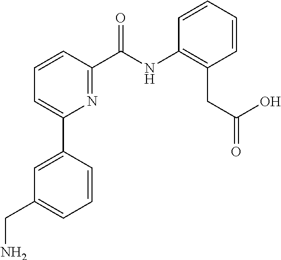 [SMILES: NCc1cccc(-c2cccc(C(=O)Nc3ccccc3CC(=O)O)n2)c1]

The title compound was prepared from methyl 2-(2-(6-chloropicolinamido)phenyl)acetate (Intermediate 41) and 3-aminomethylphenyl boronic acid HCl (CAS #146285-80-5) using the method described in Example 38. ¹H NMR (400 MHz, DMSO-d₆) δ ppm 12.22 (br. s, 1H), 9.32 (br. s, 1H), 9.13 (br. s, 2H), 8.32 (d, J=7.5 Hz, 1H), 8.20-8.06 (m, 1H), 7.84 (d, J=8.0 Hz, 1H), 7.54-7.41 (m, 2H), 7.29-7.17 (m, 3H), 7.09 (t, J=6.9 Hz, 1H), 4.10 (s, 2H), 3.41 (s, 2H). HRMS calcd. for C₂₁H₁₉N₃O₃ (M+H)⁺ 362.1505. found 362.1502.

### Example 58

### Example 58-A

### tert-Butyl ((3′-amino-[1,1′-biphenyl]-3-yl)methyl)carbamate

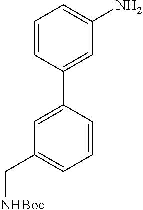 [SMILES: CC(C)(C)OC(=O)NCc1cccc(-c2cccc(N)c2)c1]

A degassed mixture of 3-bromoaniline (600 mg, 3.49 mmol), 3-((tert-butoxycarbonylamino)methyl)phenylboronic acid (1051 mg, 4.19 mmol), Pd(PPh₃)₄ (202 mg, 0.174 mmol), and 2M aq. K₃PO₄ (1481 mg, 6.98 mmol) in dioxane (15 mL) and water (3 mL) was heated at 100° C. for 1.5 hr. The reaction mixture was diluted with EtOAc and washed with water. The aqueous layer was washed with EtOAc. The combined organics were washed with brine, dried (Na₂SO₄) and concentrated. The residue was purified by silica gel chromatography (EtOAc-heptane 0-100%) to provide the title compound. ¹H NMR (400 MHz, DMSO-d₆) δ ppm 7.30-7.51 (m, 4H) 7.18 (d, J=7.20 Hz, 1H) 7.09 (t, J=7.77 Hz, 1H) 6.81 (t, J=1.83 Hz, 1H) 6.74 (d, J=7.58 Hz, 1H) 6.55 (dt, J=6.92, 1.15 Hz, 1H) 5.14 (s, 2H) 4.17 (d, J=6.06 Hz, 2H) 1.40 (s, 9H).

### Example 58-B

### Methyl 2-(2-(((3′-(((tert-butoxycarbonyl)amino)methyl)-[1,1′-biphenyl]-3-yl)amino)methyl)phenyl)acetate

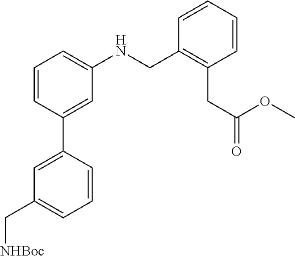 [SMILES: COC(=O)Cc1ccccc1CNc1cccc(-c2cccc(CNC(=O)OC(C)(C)C)c2)c1]

Sodium triacetoxyborohydride (111 mg, 0.523 mmol) was added to a solution of tert-butyl ((3′-amino-[1,1′-biphenyl]-3-yl)methyl)carbamate (104 mg, 0.349 mmol) and methyl 2-(2-formylphenyl)acetate (CAS #63969-83-5) (74.5 mg, 0.418 mmol) in DCE (4 mL) at rt. The reaction mixture was stirred at rt for 2.5 hr. The reaction mixture was partitioned between EtOAc and sat. aq. NH₄Cl. The aqueous layer was extracted with EtOAc. The combined organics were washed with brine, dried (Na₂SO₄) and concentrated. The residue was purified by silica gel chromatography (EtOAc-heptane 0-50%) to provide the title compound. MS (ESI⁺) m/z 461.4 (M+H).

### Example 58

### C. Methyl 2-(2-(((3′-(aminomethyl)-[1,1′-biphenyl]-3-yl)amino)methyl)phenyl)acetate

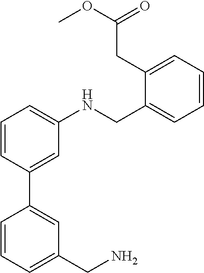 [SMILES: COC(=O)Cc1ccccc1CNc1cccc(-c2cccc(CN)c2)c1]

A solution of 2-(2-(((3′-(((tert-butoxycarbonyl)amino)methyl)-[1,1′-biphenyl]-3-yl)amino)methyl)phenyl)acetate (110 mg, 0.239 mmol) in DCM (1 ml) and TFA (1 ml) was stirred at room temperature for 15 hour. DCM (10 mL) and sat. aqueous NaHCO₃ solution (15 mL) was added. Then solid NaHCO₃ was added until pH˜8. The two layers were separated and the organic layer was concentrated. The residue was used on next step without further purification. MS (ESI+) m/z 361.3 (M+H).

### Example 58-D

### 2-(2-(((3′-(Aminomethyl)-[1,1′-biphenyl]-3-yl)amino)methyl)phenyl)acetic acid

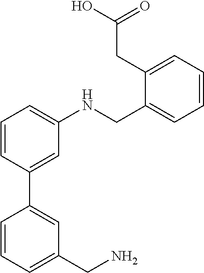 [SMILES: NCc1cccc(-c2cccc(NCc3ccccc3CC(=O)O)c2)c1]

To a solution of methyl 2-(2-(((3′-(aminomethyl)-[1,1′-biphenyl]-3-yl)amino)methyl)phenyl)acetate (86 mg, 0.239 mmol) in THF (1 ml) was added LiOH (2M solution, 0.598 ml, 1.195 mmol). The mixture was stirred at room temperature for 2 hours. The mixture was evaporated and the residue was diluted in 2 mL water. HCl was added until pH=4 and the aqueous layer was extracted with EtOAc. Then NH₄OH was added until pH=10 and the aqueous layer was extracted with EtOAc. The organic layer was dried (sodium sulfate), filtered evaporated and purified by reverse phase HPLC (Method B) to give the title compound. ¹H NMR (400 MHz, METHANOL-d₄) δ ppm 7.80 (s, 1H) 7.52-7.61 (m, 1H) 7.30-7.41 (m, 2H) 7.03-7.26 (m, 5H) 6.85-6.91 (m, 1H) 6.81 (t, J=1.96 Hz, 1H) 6.77 (ddd, J=8.02, 2.34, 0.88 Hz, 1H) 4.52 (s, 2H) 4.09 (s, 2H) 3.59 (s, 2H). HRMS calcd. for C₂₂H₂₂N₂O₂ (M+H)⁺ 347.1760. found 347.1756.

### Example 59

### Example 59-A

### Methyl 2-(2-(3-bromo-5-hydroxybenzamido)phenyl)acetate

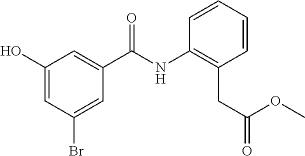 [SMILES: COC(=O)Cc1ccccc1NC(=O)c1cc(O)cc(Br)c1]

TEA (2.248 mL, 16.13 mmol) was added to a mixture of 3-bromo-5-hydroxybenzoic acid (CAS #140472-69-1) (1000 mg, 4.61 mmol), methyl 2-(2-aminophenyl)acetate hydrochloride (CAS #49851-36-7) (978 mg, 4.61 mmol) and HATU (1927 mg, 5.07 mmol) in DMF (10 mL) at 23° C. The resulting mixture was stirred at rt overnight. To the mixture was added K₂CO₃ (1911 mg, 13.82 mmol) and the mixture was heated to 100° C. for 2 hours. The reaction mixture was diluted with EtOAc and washed with water. The organic layer was concentrated and the residue was purified by flash column on silica gel (EtOAc-heptane 0-100%) to give the title compound. MS (ESI+) m/z 364.1, 366.1 (M+1).

### Example 59-B

### 2-(2-(3′-(((tert-Butoxycarbonyl)amino)methyl)-5-hydroxy-[1,1′-biphenyl]-3-ylcarboxamido)phenyl)acetic acid

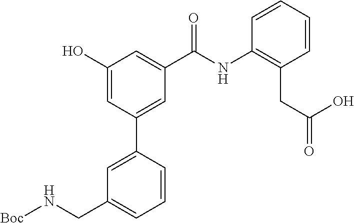 [SMILES: CC(C)(C)OC(=O)NCc1cccc(-c2cc(O)cc(C(=O)Nc3ccccc3CC(=O)O)c2)c1]

To a microwave vial was placed methyl 2-(2-(3-bromo-5-hydroxybenzamido)phenyl)acetate (40 mg, 0.110 mmol) and 3-((tert-butoxycarbonylamino)methyl)phenylboronic acid (33.1 mg, 0.132 mmol) in MeCN (2 ml). Then 2M aq. K₃PO₄ (0.16 mL, 0.329 mmol) and PdCl₂(dppf).CH₂Cl₂ adduct (CAS #95464-05-4) (4.48 mg, 5.49 μmol) were added. The vial was sealed and heated in a microwave at 110° C. for 60 min. The reaction mixture was cooled to rt, and the organic layer was filtered through a HPLC filter. The filtrate was loaded directly onto an HPLC (Method B). The fractions containing the desired product were pooled and lyophilized to provide the desired title compound. MS (ESI+) m/z 477.4 (M+H).

### Example 59-C

### 2-(2-(3′-(Aminomethyl)-5-hydroxy-[1,1′-biphenyl]-3-ylcarboxamido)phenyl)acetic acid

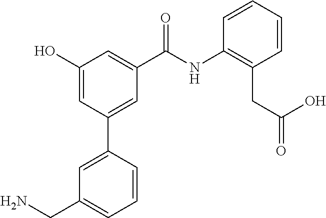 [SMILES: NCc1cccc(-c2cc(O)cc(C(=O)Nc3ccccc3CC(=O)O)c2)c1]

The title compound was synthesized from 2-(2-(3′-(((tert-butoxycarbonyl)amino)methyl)-5-hydroxy-[1,1′-biphenyl]-3-ylcarboxamido)phenyl)acetic acid using the same procedures as in Example 58-C. ¹H NMR (400 MHz, METHANOL-d₄) δ ppm 7.92 (d, J=7.33 Hz, 1H) 7.82 (t, J=1.45 Hz, 1H) 7.70 (s, 1H) 7.58 (d, J=7.83 Hz, 1H) 7.47-7.51 (m, 1H) 7.41 (t, J=7.64 Hz, 1H) 7.21-7.35 (m, 4H) 7.10 (td, J=7.45, 1.26 Hz, 1H) 3.88 (s, 2H) 3.58 (s, 2H). HRMS calcd. for C₂₂H₂₀N₂O₄ (M+H)⁺ 377.1501. found 377.1492.

### Example 60

### 2-(2-(3′-(Aminomethyl)-5′-fluoro-5-hydroxy-[1,1′-biphenyl]-3-ylcarboxamido)phenyl)acetic acid

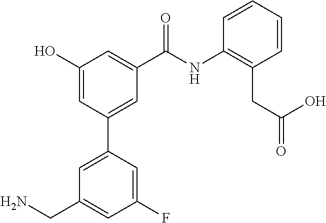 [SMILES: NCc1cc(F)cc(-c2cc(O)cc(C(=O)Nc3ccccc3CC(=O)O)c2)c1]

The title compound was synthesized from methyl 2-(2-(3-bromo-5-hydroxybenzamido)phenyl)acetate (Example 59-A) and tert-butyl 3-fluoro-5-(4,4,5,5-tetramethyl-1,3,2-dioxaborolan-2-yl)benzylcarbamate (Intermediate 24) as in Example 59. ¹H NMR (400 MHz, METHANOL-d₄) δ ppm 7.90-7.97 (m, 2H) 7.76 (s, 1H) 7.52 (dd, J=2.27, 1.64 Hz, 1H) 7.41-7.47 (m, 1H) 7.23-7.33 (m, 3H) 7.18 (d, J=8.84 Hz, 1H) 7.12 (td, J=7.48, 1.33 Hz, 1H) 4.15 (s, 2H) 3.61 (s, 2H). HRMS calcd. for C₂₂H₁₉FN₂O₄(M+H)⁺ 395.1407. found 395.1391.

### Example 61

### Example 61-A

### Methyl 2-(2-(3-bromo-5-(pyrimidin-2-ylmethoxy)benzamido)phenyl)acetate

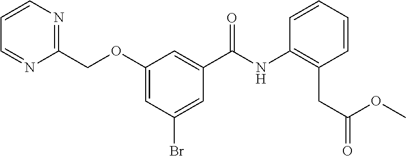 [SMILES: COC(=O)Cc1ccccc1NC(=O)c1cc(Br)cc(OCc2ncccn2)c1]

To a solution of methyl 2-(2-(3-bromo-5-hydroxybenzamido)phenyl)acetate (Example 59-A) (75 mg, 0.206 mmol), pyrimidin-2ylmethanol (CAS #42839-09-8) (45.4 mg, 0.412 mmol) and triphenylphosphine (108 mg, 0.412 mmol) in THF (3 mL) at 0° C. was added DEAD (40% in toluene) (0.163 ml, 0.412 mmol) dropwise. Then the reaction mixture was stirred at room temperature for 16 hours. The mixture was diluted in EtOAc and washed with water. The organic layer was concentrated and the residue was purified by flash column on silica gel (EtOAc-heptane 0 to 100%) to give the title compound. MS (ESI+) m/z 456.3, 458.3 (M+1).

### Example 61-B

### 2-(2-(3′-(Aminomethyl)-5-(pyrimidin-2-ylmethoxy)-[1,1′-biphenyl]-3-ylcarboxamido)phenyl)acetic acid

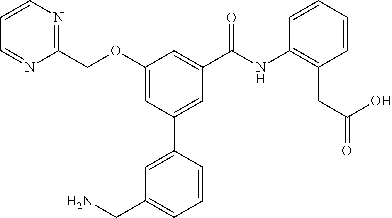 [SMILES: NCc1cccc(-c2cc(OCc3ncccn3)cc(C(=O)Nc3ccccc3CC(=O)O)c2)c1]

To a microwave vial was placed methyl 2-(2-(3-bromo-5-(pyrimidin-2-ylmethoxy)benzamido)phenyl)acetate (35 mg, 0.077 mmol) and (3-(aminomethyl)phenyl)boronic acid hydrochloride (CAS #146285-80-5) (14.38 mg, 0.077 mmol) in MeCN (2 mL). Then 2M aq. K₃PO₄ (0.12 mL, 0.24 mmol) and PdCl₂(dppf).CH₂Cl₂ adduct (CAS #95464-05-4) (3.13 mg, 3.84 μmol) were added and the vial was sealed and heated in a microwave at 110° C. for 60 min. The reaction mixture was cooled to rt and loaded directly onto an HPLC (Method B). The fractions containing the desired product were pooled and lyophilized to provide the desired product. ¹H NMR (400 MHz, METHANOL-d₄) δ ppm 8.84 (d, J=4.93 Hz, 2H) 8.10 (s, 1H) 7.92 (m, 1H) 7.68-7.71 (m, 1H) 7.37-7.57 (m, 3H) 7.23-7.33 (m, 2H) 7.08-7.18 (m, 2H) 6.66-6.83 (m, 2H) 5.45 (s, 2H) 4.11 (s, 2H) 3.60 (s, 2H). HRMS calcd. for C₂₇H₂₄N₄O₄ (M+H)⁺ 469.1876. found 469.1858.

### Example 62

### 2-(2-(3′-(Aminomethyl)-5-(pyridin-4-ylmethoxy)-[1,1′-biphenyl]-3-ylcarboxamido)phenyl)acetic acid

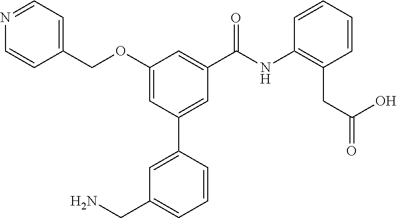 [SMILES: NCc1cccc(-c2cc(OCc3ccncc3)cc(C(=O)Nc3ccccc3CC(=O)O)c2)c1]

The title compound was synthesized in a similar manner as described in Example 61 using pyridin-4-ylmethanol (CAS #586-95-8) in place of pyrimidin-2-ylmethanol. ¹H NMR (400 MHz, METHANOL-d₄) δ ppm 8.51-8.60 (m, 2H) 8.10 (s, 1H) 7.94 (d, J=7.33 Hz, 1H) 7.89 (s, 1H) 7.68-7.75 (m, 2H) 7.61 (d, J=6.19 Hz, 2H) 7.45-7.56 (m, 2H) 7.40 (d, J=7.20 Hz, 1H) 7.22-7.35 (m, 2H) 7.12 (td, J=7.45, 1.26 Hz, 1H) 5.36 (s, 2H) 4.08 (s, 2H) 3.60 (s, 2H). HRMS calcd. for C₂₈H₂₅FN₃O₄(M+H)⁺ 468.1917. found 468.1912.

### Example 63

### (±)-2-(2-(3′-(Aminomethyl)-5-((tetrahydrofuran-2-yl)methoxy)-[1,1′-biphenyl]-3-ylcarboxamido)phenyl)acetic acid

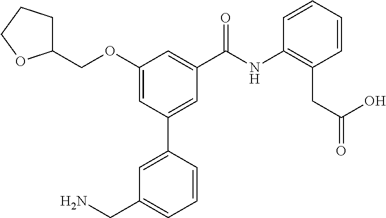 [SMILES: NCc1cccc(-c2cc(OCC3CCCO3)cc(C(=O)Nc3ccccc3CC(=O)O)c2)c1]

The title compound was synthesized in a similar manner as described in Example 61 using (±)-(tetrahydrofuran-2-yl)methanol (CAS #97-99-4). ¹H NMR (400 MHz, METHANOL-d₄) δ ppm 8.08 (t, J=1.45 Hz, 1H) 7.90-8.00 (m, 2H) 7.76 (d, J=7.83 Hz, 1H) 7.63 (dd, J=2.40, 1.52 Hz, 1H) 7.52 (t, J=7.71 Hz, 1H) 7.45 (dd, J=2.40, 1.52 Hz, 1H) 7.42 (d, J=7.83 Hz, 1H) 7.24-7.34 (m, 2H) 7.09-7.16 (m, 1H) 4.33 (qd, J=6.80, 3.73 Hz, 1H) 4.14-4.21 (m, 3H) 4.06-4.13 (m, 1H) 3.90-3.98 (m, 1H) 3.84 (td, J=7.64, 6.06 Hz, 1H) 3.62 (s, 2H) 1.80-2.20 (m, 4H). HRMS calcd. for C₂₇H₂₈N₂O₅ (M+H)⁺ 461.2076. found 461.2065.

### Example 64

### (±)-2-(2-(3′-(Aminomethyl)-5-(1-cyanoethoxy)-[1,1′-biphenyl]-3-ylcarboxamido)phenyl)acetic acid

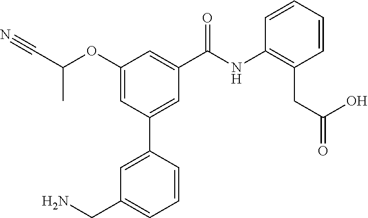 [SMILES: CC(C#N)Oc1cc(C(=O)Nc2ccccc2CC(=O)O)cc(-c2cccc(CN)c2)c1]

The title compound was synthesized in a similar manner as described in Example 61 using (±)-2-hydroxypropanenitrile (CAS #78-97-7). ¹H NMR (400 MHz, METHANOL-d₄) δ ppm 8.20-8.25 (m, 1H) 7.93-8.01 (m, 2H) 7.75-7.80 (m, 2H) 7.51-7.59 (m, 2H) 7.44 (d, J=7.83 Hz, 1H) 7.25-7.34 (m, 2H) 7.09-7.16 (m, 1H) 5.44 (q, J=6.69 Hz, 1H) 4.18 (s, 2H) 3.63 (s, 2H) 1.81 (d, J=6.69 Hz, 3H). HRMS calcd. for C₂₅H₂₃N₃O₄ (M+H)⁺ 430.1767. found 430.1749.

### Example 65

### 2-(2-(3′-(Aminomethyl)-5′-fluoro-5-(pyridin-4-ylmethoxy)-[1,1′-biphenyl]-3-ylcarboxamido)phenyl)acetic acid

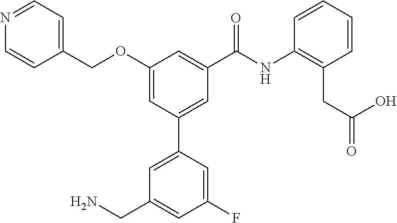 [SMILES: NCc1cc(F)cc(-c2cc(OCc3ccncc3)cc(C(=O)Nc3ccccc3CC(=O)O)c2)c1]

The title compound was synthesized as in Example 62, using tert-butyl 3-fluoro-5-(4,4,5,5-tetramethyl-1,3,2-dioxaborolan-2-yl)benzylcarbamate (Intermediate 24) in place of (3-(aminomethyl)phenyl)boronic acid. The coupled product (15 mg, 0.026 mmol) in THF (1 mL) was treated with HCl (4M in dioxane, 0.256 mmol, 0.064 mL) and stirred at 50° C. for 2 h. The crude was purified using HPLC (Method B) to afford the title compound. ¹H NMR (400 MHz, METHANOL-d₄) δ ppm 8.54-8.58 (m, 2H) 8.12 (s, 1H) 7.94 (d, J=7.83 Hz, 1H) 7.78 (s, 1H) 7.73-7.76 (m, 1H) 7.60 (d, J=6.19 Hz, 2H) 7.55 (dd, J=2.34, 1.58 Hz, 1H) 7.51 (d, J=9.73 Hz, 1H) 7.24-7.33 (m, 2H) 7.20 (d, J=8.46 Hz, 1H) 7.12 (td, J=7.52, 1.26 Hz, 1H) 5.37 (s, 2H) 4.15 (s, 2H) 3.61 (s, 2H). HRMS calcd. for C₂₈H₂₄FN₃O₄(M+H)⁺486.1824. found 486.1843.

### Example 66

### 2-(2-(3′-(Aminomethyl)-5-((1-methyl-1H-pyrazol-3-yl)methoxy)-[1,1′-biphenyl]-3-ylcarboxamido)phenyl)acetic acid

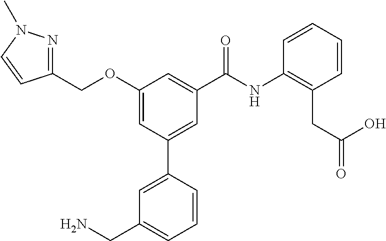 [SMILES: Cn1ccc(COc2cc(C(=O)Nc3ccccc3CC(=O)O)cc(-c3cccc(CN)c3)c2)n1]

The title compound was synthesized in a similar manner as described in Example 61, using (1-methyl-1H-pyrazol-3-yl)methanol (CAS #84547-62-6) in place of pyrimidin-2ylmethanol. ¹H NMR (400 MHz, METHANOL-d₄) δ ppm 8.06 (s, 1H) 7.93 (d, J=8.21 Hz, 1H) 7.87 (s, 1H) 7.67-7.73 (m, 2H) 7.56 (d, J=2.27 Hz, 1H) 7.45-7.51 (m, 2H) 7.38 (d, J=7.58 Hz, 1H) 7.24-7.33 (m, 2H) 7.09-7.15 (m, 1H) 6.41 (d, J=2.27 Hz, 1H) 5.20 (s, 2H) 4.06 (s, 2H) 3.89 (s, 3H) 3.61 (s, 2H). HRMS calcd. for C₂₇H₂₆N₄O₄ (M+H)⁺ 471.2032. found 471.2011.

### Example 67

### Example 67-A

### Methyl 2-(2-(3-acetamido-5-bromobenzamido)phenyl)acetate

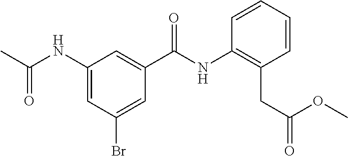 [SMILES: COC(=O)Cc1ccccc1NC(=O)c1cc(Br)cc(NC(C)=O)c1]

TEA (0.324 mL, 2.325 mmol) was added to a mixture of 3-acetamido-5-bromobenzoic acid (CAS #78238-11-6) (200 mg, 0.775 mmol) and HATU (324 mg, 0.852 mmol) in DMF (3 mL) at 23° C. After 5 min, methyl 2-(2-aminophenyl)acetate hydrochloride (CAS #49851-36-7) (165 mg, 0.775 mmol) was added and the resulting mixture was stirred at room temperature overnight. The mixture was partitioned between 4:1 EtOAc/heptanes and water. The aqueous layer was extracted with 4:1 EtOAc/heptanes. The combined organic layers were washed with 5% aqueous LiCl solution, the organics were dried (Na₂SO₄), filtered and concentrated. The residue was purified by silica gel chromatography (EtOAc/heptanes 20-80%) to provide the title compound. MS (ESI+) m/z 405.2, 407.2 (M+H).

### Example 67-B

### 2-(2-(5-Acetamido-3′-(aminomethyl)-5′-fluoro-[1,1′-biphenyl]-3-ylcarboxamido)phenyl)acetic acid

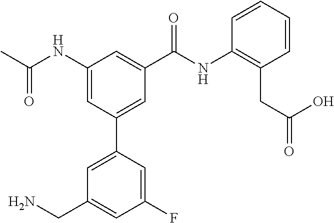 [SMILES: CC(=O)Nc1cc(C(=O)Nc2ccccc2CC(=O)O)cc(-c2cc(F)cc(CN)c2)c1]

The title compound was synthesized from methyl 2-(2-(3-acetamido-5-bromobenzamido)phenyl)acetate as described in Example 65. ¹H NMR (400 MHz, METHANOL-d₄) δ ppm 8.33 (s, 1H) 8.19 (s, 1H) 8.07 (d, J=1.64 Hz, 1H) 7.94 (d, J=7.71 Hz, 1H) 7.79 (s, 1H) 7.49 (d, J=9.73 Hz, 1H) 7.19-7.34 (m, 3H) 7.09-7.16 (m, 1H) 4.22 (s, 2H) 3.62 (s, 2H) 2.19 (s, 3H). HRMS calcd. for C₂₄H₂₂FN₃O₄(M+H)⁺ 436.1673. found 436.1661.

### Example 67-C

### Ethyl 2-(2-(5-acetamido-3′-(aminomethyl)-5′-fluoro-[1,1′-biphenyl]-3-ylcarboxamido)phenyl)acetate

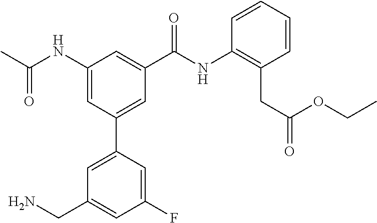 [SMILES: CCOC(=O)Cc1ccccc1NC(=O)c1cc(NC(C)=O)cc(-c2cc(F)cc(CN)c2)c1]

To a vial was placed 2-(2-(5-acetamido-3′-(aminomethyl)-5′-fluoro-[1,1′-biphenyl]-3-ylcarboxamido)phenyl)acetic acid (215 mg, 0.494 mmol) in EtOH (2 mL). Then 4N HCl in dioxane (1.235 mL, 4.94 mmol) was added and the vial was sealed and stirred at 23° C. for 3 hr. The mixture was concentrated. The residue was purified by HPLC (Method B). The fractions containing the desired product were pooled and lyophilized to provide the desired product. ¹H NMR (400 MHz, METHANOL-d₄) δ ppm 8.11 (d, J=1.64 Hz, 2H) 7.97 (s, 1H) 7.51-7.57 (m, 2H) 7.32-7.40 (m, 3H) 7.24-7.30 (m, 1H) 7.17 (d, J=9.47 Hz, 1H) 4.10 (q, J=7.12 Hz, 2H) 3.90 (s, 2H) 3.77 (s, 2H) 2.19 (s, 3H) 1.13 (t, J=7.14 Hz, 3H). HRMS calcd. for C₂₄H₂₂FN₃O₄ (M+H)⁺ 464.1980. found 464.1959.

### Example 68

### 2-(2-(3′-(Aminomethyl)-5′-fluoro-5-(pyrimidin-2-ylmethoxy)-[1,1′-biphenyl]-3-ylcarboxamido)phenyl)acetic acid

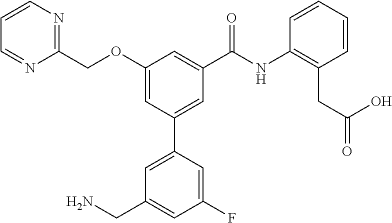 [SMILES: NCc1cc(F)cc(-c2cc(OCc3ncccn3)cc(C(=O)Nc3ccccc3CC(=O)O)c2)c1]

The title compound was synthesized in a similar manner as described in Example 65 using pyrimidin-2-ylmethanol in place of pyridin-4-ylmethanol. ¹H NMR (400 MHz, METHANOL-d₄) δ ppm 8.84 (d, J=4.93 Hz, 2H) 8.12 (t, J=1.45 Hz, 1H) 7.91 (d, J=8.21 Hz, 1H) 7.84 (s, 1H) 7.71 (dd, J=2.46, 1.45 Hz, 1H) 7.50-7.58 (m, 2H) 7.46 (t, J=4.99 Hz, 1H) 7.24-7.33 (m, 2H) 7.22 (d, J=8.84 Hz, 1H) 7.09-7.16 (m, 1H) 5.45 (s, 2H) 4.22 (s, 2H) 3.62 (s, 2H). HRMS calcd. for C₂₇H₂₃FN₄O₄(M+H)⁺ 487.1776. found 487.1767.

### Example 69

### 2-(2-(3′-(Aminomethyl)-5′-fluoro-5-((1-methyl-1H-pyrazol-3-yl)methoxy)-[1,1′-biphenyl]-3-ylcarboxamido)phenyl)acetic acid

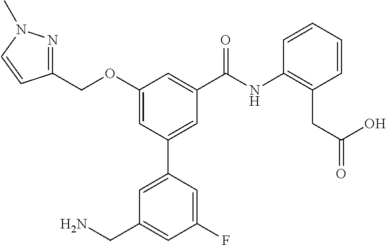 [SMILES: Cn1ccc(COc2cc(C(=O)Nc3ccccc3CC(=O)O)cc(-c3cc(F)cc(CN)c3)c2)n1]

The title compound was synthesized in a similar manner as described in Example 65 using (1-methyl-1H-pyrazol-3-yl)methanol in place of pyridin-4-ylmethanol. ¹H NMR (400 MHz, METHANOL-d₄) δ ppm 8.10 (t, J=1.45 Hz, 1H) 7.94 (d, J=7.20 Hz, 1H) 7.83 (s, 1H) 7.72 (dd, J=2.34, 1.58 Hz, 1H) 7.49-7.59 (m, 3H) 7.25-7.33 (m, 2H) 7.21 (d, J=9.09 Hz, 1H) 7.09-7.16 (m, 1H) 6.41 (d, J=2.27 Hz, 1H) 5.21 (s, 2H) 4.22 (s, 2H) 3.90 (s, 3H) 3.62 (s, 2H). HRMS calcd. for C₂₇H₂₅FN₄O₄(M+H)⁺ 489.1933. found 489.1922.

### Example 70

### 2-(2-(3′-(Aminomethyl)-5-((2-(methylcarbamoyl)pyridin-4-yl)methoxy)-[1,1′-biphenyl]-3-ylcarboxamido)phenyl)acetic acid

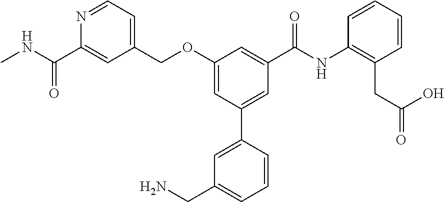 [SMILES: CNC(=O)c1cc(COc2cc(C(=O)Nc3ccccc3CC(=O)O)cc(-c3cccc(CN)c3)c2)ccn1]

The title compound was synthesized in a similar manner as described in Example 61 using 4-(hydroxymethyl)-N-methylpicolinamide (CAS #332013-43-1) in place of pyrimidin-2ylmethanol. ¹H NMR (400 MHz, METHANOL-d₄) δ ppm 8.63 (d, J=4.93 Hz, 1H) 8.25 (s, 1H) 8.11 (s, 1H) 7.89-7.96 (m, 2H) 7.68-7.76 (m, 3H) 7.56 (dd, J=2.40, 1.52 Hz, 1H) 7.51 (t, J=7.64 Hz, 1H) 7.41 (d, J=7.20 Hz, 1H) 7.24-7.32 (m, 2H) 7.12 (td, J=7.48, 1.20 Hz, 1H) 5.41 (s, 2H) 4.10 (s, 2H) 3.60 (s, 2H) 2.97 (s, 3H). HRMS calcd. for C₃₀H₂₈N₄O₅ (M+H)⁺ 525.2138. found 525.2123.

### Example 71

### 2-(2-(3′-(Aminomethyl)-5′-fluoro-5-((2-(methylcarbamoyl)pyridin-4-yl)methoxy)-[1,1′-biphenyl]-3-ylcarboxamido)phenyl)acetic acid

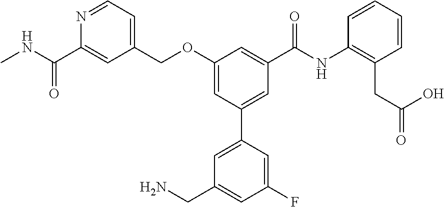 [SMILES: CNC(=O)c1cc(COc2cc(C(=O)Nc3ccccc3CC(=O)O)cc(-c3cc(F)cc(CN)c3)c2)ccn1]

The title compound was synthesized in a similar manner as described in Example 65 using 4-(hydroxymethyl)-N-methylpicolinamide in place of pyridin-4-ylmethanol. ¹H NMR (400 MHz, METHANOL-d₄) δ ppm 8.63 (dd, J=5.05, 0.76 Hz, 1H) 8.24 (d, J=0.88 Hz, 1H) 8.12 (s, 1H) 7.94 (d, J=8.34 Hz, 1H) 7.74-7.78 (m, 2H) 7.70 (d, J=4.93 Hz, 1H) 7.55-7.59 (m, 1H) 7.51 (d, J=10.11 Hz, 1H) 7.24-7.33 (m, 2H) 7.19 (d, J=8.97 Hz, 1H) 7.12 (td, J=7.48, 1.33 Hz, 1H) 5.41 (s, 2H) 4.13 (s, 2H) 3.60 (s, 2H) 2.97 (s, 3H). HRMS calcd. for C₃₀H₂₇FN₄O₅ (M+H)⁺ 543.2038. found 543.2032.

### Example 72

### Example 72-A

### Methyl 2-(2-(3-bromo-5-(2-methoxyethoxy)benzamido)phenyl)acetate

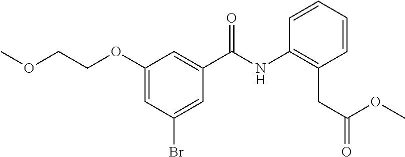

The title compound was synthesized in a similar manner as described in Example 61-A. MS (ESI+) m/z 422.3, 424.3 (M+1).

### Example 72-B

### 2-(2-(3′-(Aminomethyl)-5-(2-methoxyethoxy)-[1,1′-biphenyl]-3-ylcarboxamido)phenyl)acetic acid

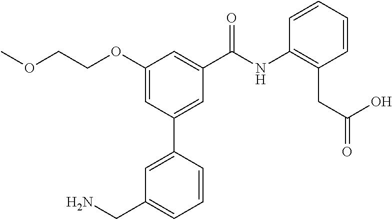 [SMILES: COCCOc1cc(C(=O)Nc2ccccc2CC(=O)O)cc(-c2cccc(CN)c2)c1]

To a microwave vial was added methyl 2-(2-(3-bromo-5-(2-methoxyethoxy)benzamido)phenyl)acetate (500 mg, 1.184 mmol) and (3-(aminomethyl)phenyl)boronic acid hydrochloride (CAS #146285-80-5) (289 mg, 1.539 mmol) in MeCN (10 mL). Then K₃PO₄ (2M aq. solution, 2.96 ml, 5.92 mmol) and Xphos palladacycle (43.7 mg, 0.059 mmol) were added and the vial was sealed and heated in a microwave at 110° C. for 60 min. The reaction mixture was cooled to rt. Saturated aq. NH₄Cl solution (3 mL) was added and the product precipitated. The solid was collected by filtration and then dissolved in a mixture of MeOH, water and DMSO. The mixture was filtered through a HPLC filter and the filtrate was loaded directly onto an HPLC (Method B). The fractions containing the desired product were pooled and lyophilized to provide the desired product. ¹H NMR (400 MHz, METHANOL-d₄) δ ppm 8.08 (t, J=1.45 Hz, 1H) 7.91-8.00 (m, 2H) 7.75 (d, J=8.21 Hz, 1H) 7.63 (dd, J=2.40, 1.52 Hz, 1H) 7.52 (t, J=7.71 Hz, 1H) 7.39-7.47 (m, 2H) 7.24-7.33 (m, 2H) 7.12 (td, J=7.45, 1.26 Hz, 1H) 4.24-4.32 (m, 2H) 4.18 (s, 2H) 3.77-3.85 (m, 2H) 3.62 (s, 2H) 3.45 (s, 3H). HRMS calcd. for C₂₅H₂₆N₂O₅ (M+H)⁺ 435.1920. found 435.1914.

### Example 73

### Example 73-A

### 2-(2-(3′-(((tert-Butoxycarbonyl)amino)methyl)-5′-fluoro-5-(2-methoxyethoxy)-[1,1′-biphenyl]-3-ylcarboxamido)phenyl)acetic acid

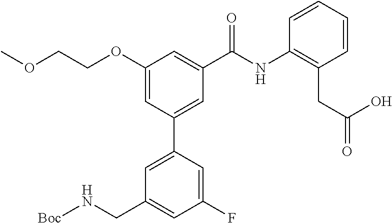 [SMILES: COCCOc1cc(C(=O)Nc2ccccc2CC(=O)O)cc(-c2cc(F)cc(CNC(=O)OC(C)(C)C)c2)c1]

The title compound was synthesized in a similar manner as described in Example 72 using tert-butyl 3-fluoro-5-(4,4,5,5-tetramethyl-1,3,2-dioxaborolan-2-yl)benzylcarbamate (Intermediate 24) in place of (3-(aminomethyl)phenyl)boronic acid. MS (ESI+) m/z 553.6 (M+H).

### Example 73-B

### Methyl 2-(2-(3′-(aminomethyl)-5′-fluoro-5-(2-methoxyethoxy)-[1,1′-biphenyl]-3-ylcarboxamido)phenyl)acetate

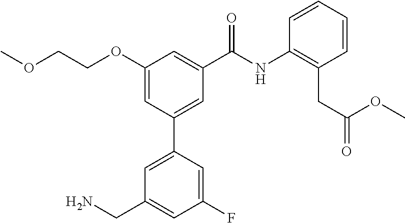

A solution of 2-(2-(3′-(((tert-butoxycarbonyl)amino)methyl)-5′-fluoro-5-(2-methoxyethoxy)-[1,1′-biphenyl]-3-ylcarboxamido)phenyl)acetic acid (200 mg, 0.362 mmol) in HCl (1.25 M in MeOH, 2.9 mL, 3.62 mmol) was heated at 50° C. for 2 hr. The mixture was concentrated and the obtained title compound was used in the next reaction without further purification. MS (ESI+) m/z 467.5 (M+H).

### Example 73-C

### 2-(2-(3′-(Aminomethyl)-5′-fluoro-5-(2-methoxyethoxy)-[1,1′-biphenyl]-3-ylcarboxamido)phenyl)acetic acid

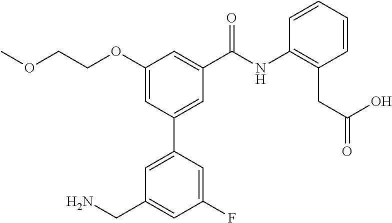 [SMILES: COCCOc1cc(C(=O)Nc2ccccc2CC(=O)O)cc(-c2cc(F)cc(CN)c2)c1]

To a solution of methyl 2-(2-(3′-(aminomethyl)-5′-fluoro-5-(2-methoxyethoxy)-[1,1′-biphenyl]-3-ylcarboxamido)phenyl)acetate (169 mg, 0.362 mmol) in THF (3 mL) was added LiOH (1.810 ml, 1.810 mmol). The mixture was stirred at room temperature for 16 h, then diluted with EtOAc and water, separated. The organic layer was washed with 5 mL of a 1N NaOH solution. The aqueous layers were combined and acidified with saturated NH₄Cl solution, then extracted with EtOAc. The combined organic layers were dried (sodium sulfate), filtered and concentrated and the residue was purified by HPLC (Method B) to provide the title compound. ¹H NMR (400 MHz, METHANOL-d₄) δ ppm 8.07 (d, J=1.39 Hz, 1H) 7.94 (d, J=7.83 Hz, 1H) 7.81 (s, 1H) 7.64 (dd, J=2.34, 1.58 Hz, 1H) 7.52 (dt, J=9.92, 1.86 Hz, 1H) 7.41-7.46 (m, 1H) 7.25-7.33 (m, 2H) 7.17-7.23 (m, 1H) 7.13 (td, J=7.52, 1.26 Hz, 1H) 4.24-4.30 (m, 2H) 4.20 (s, 2H) 3.77-3.83 (m, 2H) 3.62 (s, 2H) 3.44 (s, 3H). HRMS calcd. for C₂₅H₂₅FN₂O₅(M+H)⁺ 453.1820. found 453.1811.

### Example 74

### Example 74-A

### ((R)-Tetrahydrofuran-2-yl)methyl 3-bromo-5-(((R)-tetrahydrofuran-2-yl)methoxy)benzoate

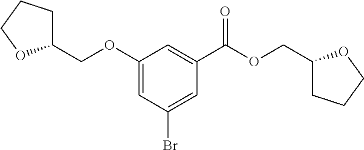 [SMILES: O=C(OC[C@H]1CCCO1)c1cc(Br)cc(OC[C@H]2CCCO2)c1]

To a solution of 3-bromo-5-hydroxybenzoic acid (CAS #140472-69-1) (1000 mg, 4.61 mmol), (R)-(tetrahydrofuran-2-yl)methanol (CAS #22415-59-4) (1035 mg, 10.14 mmol) and triphenylphosphine (3021 mg, 11.52 mmol) in THF (20 mL) at 0° C. was added DEAD (40% in toluene) (5.25 ml, 11.52 mmol) dropwise. Then the reaction mixture was stirred at room temperature for 16 hours, diluted in EtOAc and washed with water. The organic layer was concentrated and the residue was purified by flash column (EtOAc-Heptane 0-100%) to give the title compound. MS (ESI+) m/z 385.3 387.3 (M+1).

### Example 74-B

### (R)-3-Bromo-5-((tetrahydrofuran-2-yl)methoxy)benzoic acid

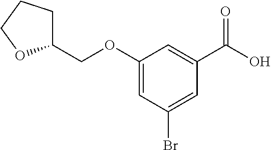 [SMILES: O=C(O)c1cc(Br)cc(OC[C@H]2CCCO2)c1]

To a solution of ((R)-tetrahydrofuran-2-yl)methyl 3-bromo-5-(((R)-tetrahydrofuran-2-yl)methoxy)benzoate (1600 mg, 4.15 mmol) in THF (20 mL) at room temperature was added LiOH (1 M solution) (12.46 ml, 12.46 mmol). Then the reaction was stirred at room temperature for 16 hours. The mixture was diluted with EtOAc and extracted with water. The aqueous layer was acidified to pH 1 with concentrated HCl. The aqueous layer was then extracted with EtOAc. The combined organic layers were dried (sodium sulfate), filtered and concentrated to give the title compound, which was used in next reaction without further purification. MS (ESI−) m/z 299.1 301.1 (M−H).

### Example 74-C

### (R)-Methyl 2-(2-(3-bromo-5-((tetrahydrofuran-2-yl)methoxy)benzamido)phenyl)acetate

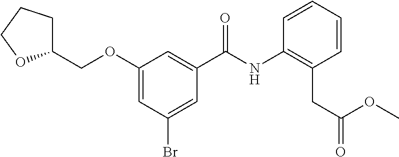 [SMILES: COC(=O)Cc1ccccc1NC(=O)c1cc(Br)cc(OC[C@H]2CCCO2)c1]

The title compound was synthesized in a similar manner as described in Example 59-A. MS (ESI+) m/z 448.3 450.3 (M+1).

### Example 74-D

### (R)-2-(2-(3′-(Aminomethyl)-5-((tetrahydrofuran-2-yl)methoxy)-[1,1′-biphenyl]-3-ylcarboxamido)phenyl)acetic acid

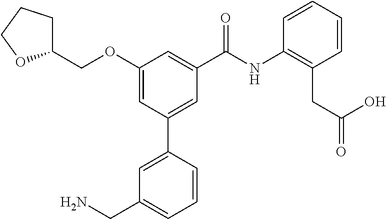 [SMILES: NCc1cccc(-c2cc(OC[C@H]3CCCO3)cc(C(=O)Nc3ccccc3CC(=O)O)c2)c1]

The title compound was synthesized in a similar manner as described in Example 72-B from (R)-methyl 2-(2-(3-bromo-5-((tetrahydrofuran-2-yl)methoxy)benzamido)phenyl)acetate. ¹H NMR (400 MHz, DMSO-d₆) δ ppm 12.73 (s, 1H) 8.78 (br. s., 2H) 8.27 (d, J=18.06 Hz, 2H) 7.98 (d, J=7.07 Hz, 1H) 7.83 (d, J=7.96 Hz, 1H) 7.44-7.56 (m, 3H) 7.39 (d, J=7.71 Hz, 1H) 7.15-7.28 (m, 2H) 7.04 (td, J=7.45, 1.26 Hz, 1H) 4.18-4.29 (m, 1H) 4.04-4.18 (m, 4H) 3.76-3.90 (m, 1H) 3.71 (td, J=7.67, 6.13 Hz, 1H) 3.51 (s, 2H) 1.65-2.14 (m, 4H). HRMS calcd. for C₂₇H₂₈N₂O₅ (M+H)⁺ 461.2076. found 461.2070.

### Example 75

### Example 75-A

### (S)-2-(2-((3′-(1-((tert-Butoxycarbonyl)amino)-2-hydroxyethyl)-[1,1′-biphenyl]-3-yl)methoxy)-3-fluorophenyl)acetic acid

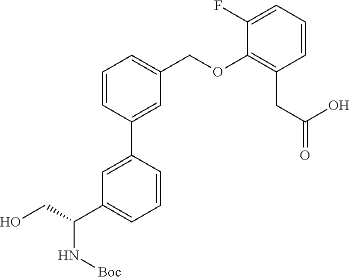 [SMILES: CC(C)(C)OC(=O)N[C@H](CO)c1cccc(-c2cccc(COc3c(F)cccc3CC(=O)O)c2)c1]

To a microwave vial was placed methyl 2-(2-((3-bromobenzyl)oxy)-3-fluorophenyl)acetate (Intermediate 90) (100 mg, 0.283 mmol) and (S)-tert-butyl (2-hydroxy-1-(3-(4,4,5,5-tetramethyl-1,3,2-dioxaborolan-2-yl)phenyl)ethyl)carbamate (Intermediate 34-B) (134 mg, 0.368 mmol) in MeCN (2 mL). Then 2M aq. K₃PO₄ (0.708 ml, 1.416 mmol) and Xphos palladacycle (10.46 mg, 0.014 mmol) were added and the vial was sealed and heated in a microwave at 110° C. for 60 min. The reaction mixture was cooled to rt, acidified with 1N HCl solution to pH˜5. The organic layer was filtered. 0.5 mL 1N LiOH was added to the filtrate and stirred overnight. HCl was added to pH˜5. The mixture was filtered and the filtrate was purified by preparative HPLC (Method B) to provide the title compound. MS (ESI−) m/z 494.2 (M−H).

### Example 75-B

### (S)-2-(2-((3′-(1-Amino-2-hydroxyethyl)-[1,1′-biphenyl]-3-yl)methoxy)-3-fluorophenyl)acetic acid

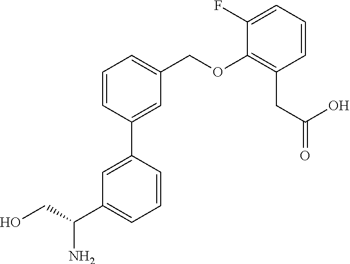 [SMILES: N[C@H](CO)c1cccc(-c2cccc(COc3c(F)cccc3CC(=O)O)c2)c1]

A solution of (S)-2-(2-((3′-(1-((tert-butoxycarbonyl)amino)-2-hydroxyethyl)-[1,1′-biphenyl]-3-yl)methoxy)-3-fluorophenyl)acetic acid (45 mg, 0.088 mmol) in DCM (1 mL) and TFA (0.680 mL, 8.83 mmol) was stirred at room temperature for 1 hr. The mixture was concentrated and the residue was purified by HPLC (Method B) to provide the title compound. ¹H NMR (400 MHz, METHANOL-d₄) δ ppm 8.11 (s, 1H) 8.07 (s, 1H) 7.69-7.73 (m, 1H) 7.63 (d, J=7.70 Hz, 1H) 7.41-7.51 (m, 2H) 7.34-7.39 (m, 2H) 6.96-7.05 (m, 3H) 5.23 (s, 2H) 4.38 (dd, J=8.53, 4.74 Hz, 1H) 3.86-3.99 (m, 2H) 3.53-3.67 (m, 2H). HRMS calcd. for C₂₃H₂₂FNO₄ (M+H)⁺ 396.1611. found 396.1600.

### Example 76

### (R)-2-(2-(3′-(Aminomethyl)-5′-fluoro-5-((tetrahydrofuran-2-yl)methoxy)-[1,1′-biphenyl]-3-ylcarboxamido)phenyl)acetic acid

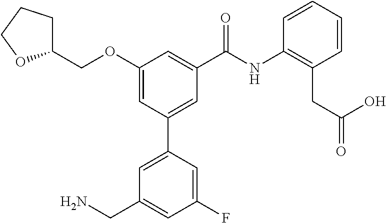 [SMILES: NCc1cc(F)cc(-c2cc(OC[C@H]3CCCO3)cc(C(=O)Nc3ccccc3CC(=O)O)c2)c1]

The title compound was synthesized in a similar manner as described in Example 73 starting from (R)-methyl 2-(2-(3-bromo-5-((tetrahydrofuran-2-yl)methoxy)benzamido)phenyl)acetate (Example 74-C). ¹H NMR (400 MHz, METHANOL-d₄) b ppm 8.06 (t, J=1.39 Hz, 1H) 7.94 (d, J=7.20 Hz, 1H) 7.80 (s, 1H) 7.63 (dd, J=2.34, 1.58 Hz, 1H) 7.52 (dt, J=9.79, 1.99 Hz, 1H) 7.42 (dd, J=2.34, 1.58 Hz, 1H) 7.24-7.33 (m, 2H) 7.09-7.21 (m, 2H) 4.31 (qd, J=6.80, 3.60 Hz, 1H) 4.12-4.22 (m, 3H) 4.04-4.11 (m, 1H) 3.89-3.98 (m, 1H) 3.83 (td, J=7.64, 5.94 Hz, 1H) 3.62 (s, 2H) 1.77-2.19 (m, 4H). HRMS calcd. for C₂₇H₂₇FN₂O₅(M+H)⁺ 479.1982. found 479.1974.

### Example 77

### Example 77-A

### tert-Butyl 2-(2-(3′-(((tert-butoxycarbonyl)amino)methyl)-5′-fluoro-5-morpholino-[1,1′-biphenyl]-3-ylcarboxamido)phenyl)acetate

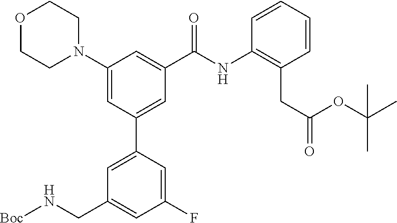 [SMILES: CC(C)(C)OC(=O)Cc1ccccc1NC(=O)c1cc(-c2cc(F)cc(CNC(=O)OC(C)(C)C)c2)cc(N2CCOCC2)c1]

A suspension of tert-butyl 2-(2-(5-bromo-3′-(((tert-butoxycarbonyl)amino)methyl)-5′-fluoro-[1,1′-biphenyl]-3-ylcarboxamido)phenyl)acetate (Example 11-B) (100 mg, 0.163 mmol), morpholine (71.0 mg, 0.815 mmol), Cs₂CO₃ (159 mg, 0.489 mmol) and RuPhos palladacycle (CAS #1028206-60-1) (5.94 mg, 8.15 μmol) in CH₃CN (2 mL) was heated in a microwave at 160° C. for 60 min. The mixture was diluted with EtOAc and water, acidified with 1N HCl solution to pH 5, extracted with EtOAc. The combined organic layers were concentrated and the residue was purified by HPLC (Method B) to provide the title compound. MS (ESI+) m/z 620.8 (M+H).

### Example 77-B

### 2-(2-(3′-(Aminomethyl)-5′-fluoro-5-morpholino-[1,1′-biphenyl]-3-ylcarboxamido)phenyl)acetic acid

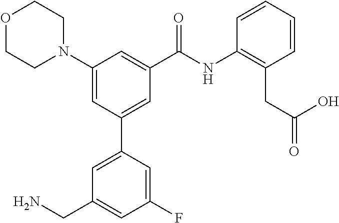 [SMILES: NCc1cc(F)cc(-c2cc(C(=O)Nc3ccccc3CC(=O)O)cc(N3CCOCC3)c2)c1]

A solution of tert-butyl 2-(2-(3′-(((tert-butoxycarbonyl)amino)methyl)-5′-fluoro-5-morpholino-[1,1′-biphenyl]-3-ylcarboxamido)phenyl)acetate (101 mg, 0.163 mmol) in DCM (2 mL) and TFA (1 mL) was stirred at 23° C. for 30 min. The reaction mixture was concentrated and the residue was purified by HPLC (Method B) to provide the title compound. ¹H NMR (400 MHz, METHANOL-d₄) δ ppm 7.91-7.97 (m, 2H) 7.81 (s, 1H) 7.65-7.69 (m, 1H) 7.55 (dt, J=10.04, 1.93 Hz, 1H) 7.41-7.44 (m, 1H) 7.25-7.32 (m, 2H) 7.20 (d, J=8.97 Hz, 1H) 7.09-7.15 (m, 1H) 4.21 (s, 2H) 3.85-3.92 (m, 4H) 3.61 (s, 2H) 3.30-3.35 (m, 4H overlap with residual MeOH signal). HRMS calcd. for C₂₆H₂₆FN₃O₄(M+H)⁺ 464.1986. found 464.1978.

### Example 78

### Example 78-A

### tert-Butyl 2-(2-(3′-(((tert-butoxycarbonyl)amino)methyl)-5-((3-cyclopropyl-1-methyl-1H-pyrazol-5-yl)amino)-5′-fluoro-[1,1′-biphenyl]-3-ylcarboxamido)phenyl)acetate

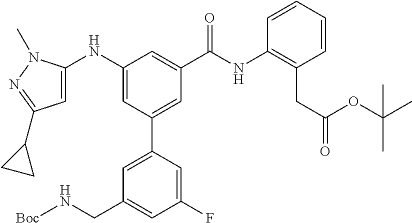 [SMILES: Cn1nc(C2CC2)cc1Nc1cc(C(=O)Nc2ccccc2CC(=O)OC(C)(C)C)cc(-c2cc(F)cc(CNC(=O)OC(C)(C)C)c2)c1]

A mixture of tert-butyl 2-(2-(5-bromo-3′-(((tert-butoxycarbonyl)amino)methyl)-5′-fluoro-[1,1′-biphenyl]-3-ylcarboxamido)phenyl)acetate (Example 11-B) (100 mg, 0.163 mmol), 3-cyclopropyl-1-methyl-1H-pyrazol-5-amine (CAS #118430-74-3) (44.7 mg, 0.326 mmol), Cs₂CO₃ (159 mg, 0.489 mmol) and BrettPhos palladacycle (CAS #1148148-01-9) (6.51 mg, 8.15 μmol) in CH₃CN (2 mL) was heated in a microwave at 160° C. for 60 min. The mixture was diluted with EtOAc and water, acidified with 1N HCl to pH 5. The layers were separated and the aqueous layer was extracted with EtOAc. The combined organic layers were concentrated and the residue was purified by HPLC (Method B) to provide the title compound. MS (ESI+) m/z 670.8 (M+H).

### Example 78-B

### 2-(2-(3′-(Aminomethyl)-5-((3-cyclopropyl-1-methyl-1H-pyrazol-5-yl)amino)-5′-fluoro-[1,1′-biphenyl]-3-ylcarboxamido)phenyl)acetic acid

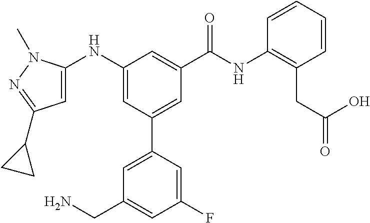 [SMILES: Cn1nc(C2CC2)cc1Nc1cc(C(=O)Nc2ccccc2CC(=O)O)cc(-c2cc(F)cc(CN)c2)c1]

The title compound was synthesized from tert-butyl 2-(2-(3′-(((tert-butoxycarbonyl)amino)methyl)-5-((3-cyclopropyl-1-methyl-1H-pyrazol-5-yl)amino)-5′-fluoro-[1,1′-biphenyl]-3-ylcarboxamido)phenyl)acetate in a similar manner as described in Example 77-B. ¹H NMR (400 MHz, METHANOL-d₄) δ ppm 7.89-7.95 (m, 2H) 7.80 (s, 1H) 7.51-7.55 (m, 1H) 7.38-7.44 (m, 1H) 7.24-7.32 (m, 3H) 7.20 (d, J=9.09 Hz, 1H) 7.09-7.15 (m, 1H) 5.84 (s, 1H) 4.21 (s, 2H) 3.66 (s, 3H) 3.61 (s, 2H) 1.87 (tt, J=8.46, 5.05 Hz, 1H) 0.85-0.95 (m, 2H) 0.67-0.75 (m, 2H). HRMS calcd. for C₂₆H₂₆FN₃O₄(M+H)⁺ 514.2249. found 514.2250.

### Example 79

The compounds in the table below were synthesized as described in Example 78.

| | Structure/Chemical Name | ¹H NMR | HRMS |
|---|---|---|---|
| 79-A | 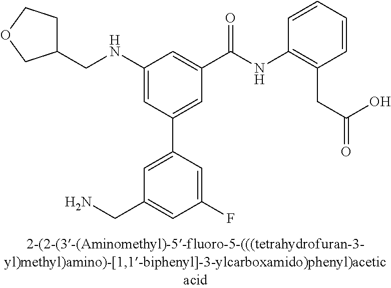 [SMILES: NCc1cc(F)cc(-c2cc(NCC3CCOC3)cc(C(=O)Nc3ccccc3CC(=O)O)c2)c1] | (400 MHz, METHANOL-d₄) δ ppm 7.92 (d, J = 7.83 Hz, 1 H) 7.78 (s, 1 H) 7.69 (s, 1 H) 7.46 (d, J = 9.73 Hz, 1 H) 7.32-7.35 (m, 1 H) 7.24-7.31 (m, 2 H) 7.17 (d, J = 9.09 Hz, 1 H) 7.12 (td, J = 7.52, 1.26 Hz, 1 H) 7.06-7.08 (m, 1 H) 4.19 (s, 2 H) 3.88-3.95 (m, 2 H) 3.77 (q, J = 7.66 Hz, 1 H) 3.64 (dd, J = 8.59, 5.56 Hz, 1 H) 3.61 (s, 2 H) 3.22 (d, J = 7.33 Hz, 2 H) 2.59-2.70 (m, 1 H) 2.10-2.20 (m, 1 H) 1.71-1.81 (m, 1 H). | calcd. for C₂₆H₂₆FN₃O₄ (M + H)⁺ 478.2137, found 478.2153. |
| 79-B | 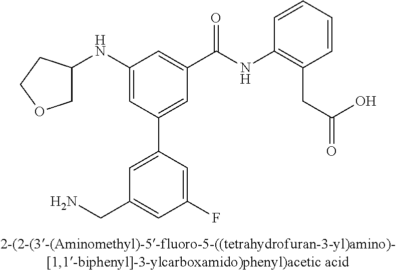 [SMILES: NCc1cc(F)cc(-c2cc(NC3CCOC3)cc(C(=O)Nc3ccccc3CC(=O)O)c2)c1] | (400 MHz, METHANOL-d₄) δ ppm 7.93 (d, J = 7.07 Hz, 1 H) 7.77 (s, 1 H) 7.71 (t, J = 1.45 Hz, 1 H) 7.43-7.49 (m, 1 H) 7.32-7.34 (m, 1 H) 7.25-7.31 (m, 2 H) 7.17 (d, J = 9.22 Hz, 1 H) 7.07-7.14 (m, 2 H) 4.23 (td, J = 6.35, 3.60 Hz, 1 H) 4.18 (s, 2 H) 4.04 (dd, J = 8.97, 5.68 Hz, 1 H) 3.95-4.01 (m, 1 H) 3.88 (td, J = 8.31, 5.37 Hz, 1 H) 3.73 (dd, J = 8.84, 3.28 Hz, 1 H) 3.61 (s, 2 H) 2.29-2.41 (m, 1 H) 1.88-1.99 (m, 1 H). | calcd. for C₂₆H₂₆FN₃O₄ (M + H)⁺ 464.1986, found 464.1969. |
| 79-C | 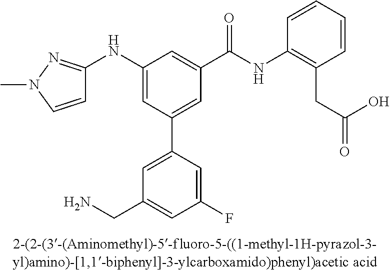 [SMILES: Cn1ccc(Nc2cc(C(=O)Nc3ccccc3CC(=O)O)cc(-c3cc(F)cc(CN)c3)c2)n1] | (400 MHz, METHANOL-d₄) δ ppm 7.93 (d, J = 7.07 Hz, 1 H) 7.80-7.88 (m, 4 H) 7.44-7.50 (m, 2 H) 7.25-7.32 (m, 2 H) 7.19 (dd, J = 9.03, 1.58 Hz, 1 H) 7.09-7.15 (m, 1 H) 5.97 (d, J = 2.27 Hz, 1 H) 4.21 (s, 2 H) 3.83 (s, 3 H) 3.62 (s, 2 H). | calcd. for C₂₆H₂₆FN₃O₄ (M + H)⁺ 474.1936, found 474.1914. |
| 79-D | 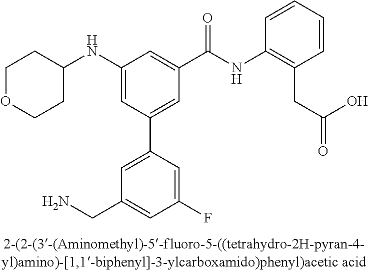 [SMILES: NCc1cc(F)cc(-c2cc(NC3CCOCC3)cc(C(=O)Nc3ccccc3CC(=O)O)c2)c1] | (400 MHz, METHANOL-d₄) δ ppm 7.92 (d, J = 7.20 Hz, 1 H) 7.78 (s, 1 H) 7.67-7.70 (m, 1 H) 7.43-7.48 (m, 1 H) 7.35 (t, J = 1.83 Hz, 1 H) 7.24-7.32 (m, 2 H) 7.17 (d, J = 8.84 Hz, 1 H) 7.09-7.14 (m, 2 H) 4.19 (s, 2 H) 3.96-4.03 (m, 2 H) 3.56-3.72 (m, 5 H) 2.06 (d, J = 12.25 Hz, 2 H) 1.48-1.60 (m, 2 H). | calcd. for C₂₇H₂₈FN₃O₄ (M + H)⁺ 478.2142, found 478.2120. |
| 79-E | 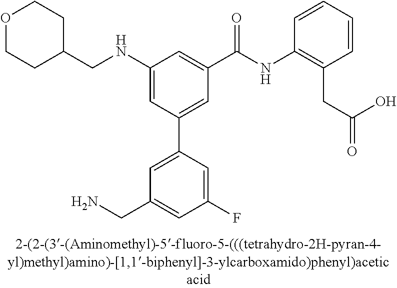 [SMILES: NCc1cc(F)cc(-c2cc(NCC3CCOCC3)cc(C(=O)Nc3ccccc3CC(=O)O)c2)c1] | (400 MHz, METHANOL-d₄) δ ppm 7.92 (d, J = 7.07 Hz, 1 H) 7.78 (s, 1 H) 7.67 (t, J = 1.45 Hz, 1 H) 7.42-7.48 (m, 1 H) 7.24-7.34 (m, 3 H) 7.17 (d, J = 8.97 Hz, 1 H) 7.12 (td, J = 7.48, 1.20 Hz, 1 H) 7.04-7.08 (m, 1 H) 4.19 (s, 2 H) 3.97 (dd, J = 11.18, 3.35 Hz, 2 H) 3.61 (s, 2 H) 3.40-3.50 (m, 2 H) 3.11-3.16 (m, 2 H) 1.88-2.02 (m, 1 H) 1.80 (d, J = 12.88 Hz, 2 H) 1.30-1.45 (m, 2 H). | calcd. for C₂₈H₃₀FN₃O₄ (M + H)⁺ 492.2299, found 492.2275. |
| 79-F | 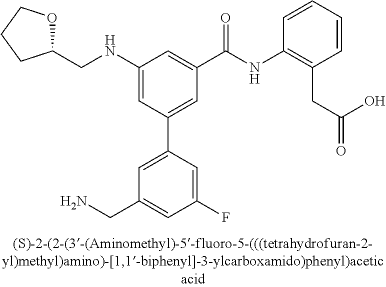 [SMILES: NCc1cc(F)cc(-c2cc(NC[C@@H]3CCCO3)cc(C(=O)Nc3ccccc3CC(=O)O)c2)c1] | (400 MHz, METHANOL-d₄) δ ppm 7.90-7.94 (m, 1 H) 7.78 (s, 1 H) 7.70 (t, J = 1.39 Hz, 1 H) 7.47 (dt, J = 9.79, 1.93 Hz, 1 H) 7.34-7.37 (m, 1 H) 7.24-7.32 (m, 2 H) 7.09-7.19 (m, 3 H) 4.12-4.21 (m, 3 H) 3.87-3.95 (m, 1 H) 3.78 (td, J = 7.71, 6.32 Hz, 1 H) 3.61 (s, 2 H) 3.32-3.38 (m, 1 H) 3.23-3.28 (m, 1 H) 1.86-2.16 (m, 3 H) 1.68-1.80 (m, 1 H). | calcd. for C₂₈H₃₀FN₃O₄ (M + H)⁺ 478.2137, found 478.2114. |
| 79-G | 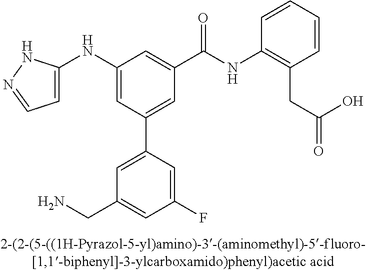 [SMILES: NCc1cc(F)cc(-c2cc(Nc3ccn[nH]3)cc(C(=O)Nc3ccccc3CC(=O)O)c2)c1] | (400 MHz, METHANOL-d₄) δ ppm 7.73-7.83 (m, 5 H) 7.57 (d, J = 2.40 Hz, 1 H) 7.52 (d, J = 10.23 Hz, 1 H) 7.28-7.36 (m, 2 H) 7.15-7.24 (m, 2 H) 6.04 (d, J = 2.40 Hz, 1 H) 4.22 (s, 2 H) 3.67 (s, 2 H). | calcd. for C₂₅H₂₂FN₅O₃ (M + H)⁺ 460.1785, found 460.1766. |
| 79-H | 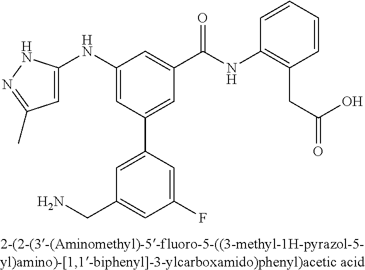 [SMILES: Cc1cc(Nc2cc(C(=O)Nc3ccccc3CC(=O)O)cc(-c3cc(F)cc(CN)c3)c2)[nH]n1] | (400 MHz, METHANOL-d₄) δ ppm 7.77 (s, 1 H) 7.72 (t, J = 1.83 Hz, 1 H) 7.66-7.70 (m, 3 H) 7.52 (d, J = 10.36 Hz, 1 H) 7.31-7.38 (m, 2 H) 7.19-7.26 (m, 2 H) 5.81 (s, 1 H) 4.22 (s, 2 H) 3.71 (s, 2 H) 2.28 (s, 3 H). | calcd. for C₂₆H₂₄FN₅O₃ (M + H)⁺ 474.1936, found 474.1914. |
| 79-I | 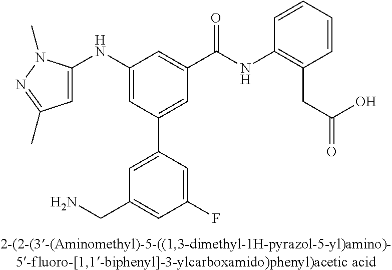 [SMILES: Cc1cc(Nc2cc(C(=O)Nc3ccccc3CC(=O)O)cc(-c3cc(F)cc(CN)c3)c2)n(C)n1] | (400 MHz, METHANOL-d₄) δ ppm 7.74 (s, 1 H) 7.70 (d, J = 7.71 Hz, 1 H) 7.63 (s, 1 H) 7.45-7.50 (m, 2 H) 7.31-7.37 (m, 2 H) 7.20-7.29 (m, 3 H) 5.97 (s, 1 H) 4.22 (s, 2 H) 3.69 (s, 2 H) 3.67 (s, 3 H) 2.22 (s, 3 H). | calcd. for C₂₇H₂₆FN₅O₃ (M + H)⁺ 488.2098, found 488.2077. |

### Example 80

### Example 80-A

### (S)-Methyl 2-(2-(3-bromo-5-((tetrahydrofuran-2-yl)methoxy)benzamido)phenyl)acetate

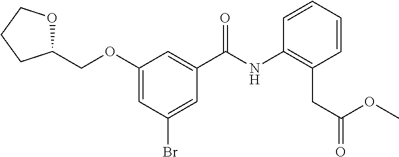 [SMILES: COC(=O)Cc1ccccc1NC(=O)c1cc(Br)cc(OC[C@@H]2CCCO2)c1]

The title compound was synthesized in a similar manner as described in Example 74-A to 74-C using (S)-(tetrahydrofuran-2-yl)methanol (CAS #57203-01-7) in place of (R)-(tetrahydrofuran-2-yl)methanol (CAS #22415-59-4). MS (ESI+) m/z 448.3 450.3 (M+1).

### Example 80-B

### (S)-Methyl 2-(2-(3-((tetrahydrofuran-2-yl)methoxy)-5-(4,4,5,5-tetramethyl-1,3,2-dioxaborolan-2-yl)benzamido)phenyl)acetate

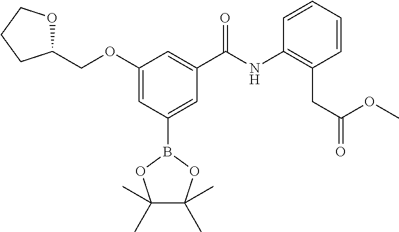 [SMILES: COC(=O)Cc1ccccc1NC(=O)c1cc(OC[C@@H]2CCCO2)cc(B2OC(C)(C)C(C)(C)O2)c1]

A degassed mixture of (S)-methyl 2-(2-(3-bromo-5-((tetrahydrofuran-2-yl)methoxy)benzamido)phenyl)acetate (200 mg, 0.446 mmol), 4,4,4′,4′,5,5,5′,5′-octamethyl-2,2′-bi(1,3,2-dioxaborolane) (CAS #73183-34-3) (227 mg, 0.892 mmol), potassium acetate (109 mg, 1.115 mmol) and Xphos Palladacycle (16.48 mg, 0.022 mmol) in MeCN (3 mL) was heated at 70° C. for 16 hour. The mixture was filtered and concentrated. The residue was purified by flash column (EtOAc-Heptane 0-100%) to give the title compound. MS (ESI+) m/z 496.5 (M+H).

### Example 80-A

### 2-(2-(3′-((R)-1-Aminoethyl)-5-(((S)-tetrahydrofuran-2-yl)methoxy)-[1,1′-biphenyl]-3-ylcarboxamido)phenyl)acetic acid

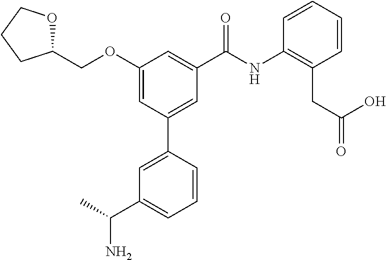 [SMILES: C[C@@H](N)c1cccc(-c2cc(OC[C@@H]3CCCO3)cc(C(=O)Nc3ccccc3CC(=O)O)c2)c1]

To a microwave vial (S)-methyl 2-(2-(3-((tetrahydrofuran-2-yl)methoxy)-5-(4,4,5,5-tetramethyl-1,3,2-dioxaborolan-2-yl)benzamido)phenyl)acetate (30 mg, 0.061 mmol) and (R)-1-(3-bromophenyl)ethanamine (CAS #176707-77-0) (36.4 mg, 0.182 mmol) in MeCN (1 ml) were added. Then 2M aq. K₃PO₄ (0.151 ml, 0.303 mmol) and Xphos palladacycle (2.2 mg, 3.03 μmol) were added and the vial was sealed and heated in a microwave at 110° C. for 60 min. The reaction mixture was cooled to rt, acidified with 1N HCl solution to pH 5. The organic layer was filtered and purified by HPLC (Method B) to provide the title compound. ¹H NMR (400 MHz, METHANOL-d₄) δ ppm 8.11 (s, 1H) 8.01 (s, 1H) 7.93 (d, J=7.58 Hz, 1H) 7.77 (d, J=8.46 Hz, 1H) 7.62-7.65 (m, 1H) 7.54 (t, J=7.77 Hz, 1H) 7.41-7.48 (m, 2H) 7.26-7.33 (m, 2H) 7.13 (td, J=7.52, 1.26 Hz, 1H) 4.54 (q, J=7.03 Hz, 1H) 4.29-4.37 (m, 1H) 4.16-4.21 (m, 1H) 4.08-4.13 (m, 1H) 3.91-3.98 (m, 1H) 3.80-3.88 (m, 1H) 3.58-3.67 (m, 2H) 1.81-2.20 (m, 4H) 1.70 (d, J=6.95 Hz, 3H). HRMS calcd. for C₂₈H₃₀FN₃O₄(M+H)⁺ 475.2233. found 475.2209.

### Example 81

### 2-(2-(3′-((S)-1-Amino-2-hydroxyethyl)-5-(((S)-tetrahydrofuran-2-yl)methoxy)-[1,1′-biphenyl]-3-ylcarboxamido)phenyl)acetic acid

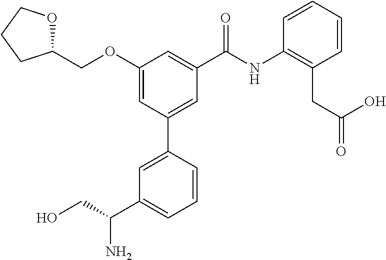 [SMILES: N[C@H](CO)c1cccc(-c2cc(OC[C@@H]3CCCO3)cc(C(=O)Nc3ccccc3CC(=O)O)c2)c1]

The title compound was synthesized in a similar manner as described in Example 80 using (S)-tert-butyl (2-hydroxy-1-(3-(4,4,5,5-tetramethyl-1,3,2-dioxaborolan-2-yl)phenyl)ethyl)carbamate (Intermediate 34-B) in place of (R)-1-(3-bromophenyl)ethanamine. Deprotection of Boc was achieved using conditions as in Example 77-B. ¹H NMR (400 MHz, METHANOL-d₄) δ ppm 8.12 (s, 1H) 8.00 (s, 1H) 7.96 (d, J=7.96 Hz, 1H) 7.78 (d, J=7.96 Hz, 1H) 7.62-7.65 (m, 1H) 7.54 (t, J=7.71 Hz, 1H) 7.45-7.48 (m, 1H) 7.42 (d, J=7.83 Hz, 1H) 7.25-7.34 (m, 2H) 7.09-7.15 (m, 1H) 4.42 (t, J=6.51 Hz, 1H) 4.33 (dq, J=6.57, 3.37 Hz, 1H) 4.15-4.21 (m, 1H) 4.07-4.13 (m, 1H) 3.89-3.98 (m, 3H) 3.80-3.88 (m, 1H) 3.56-3.67 (m, 2H) 1.81-2.19 (m, 4H). HRMS calcd. for C₂₈H₃₀N₂O₆ (M+H)⁺ 491.2182. found 491.2159.

### Example 82

### Example 82-A

### tert-Butyl 2-(2-(3,5-dibromobenzamido)phenyl)acetate

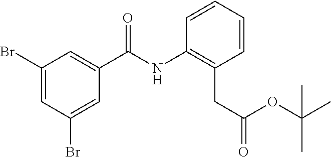 [SMILES: CC(C)(C)OC(=O)Cc1ccccc1NC(=O)c1cc(Br)cc(Br)c1]

TEA (6.72 mL, 48.2 mmol) was added to a mixture of 3,5-dibromobenzoic acid (7 g, 25.01 mmol), tert-butyl 2-(2-aminophenyl)acetate (CAS #98911-34-3) (5 g, 24.12 mmol) and HATU (9.63 g, 25.3 mmol) in DMF at 23° C. The mixture was stirred at rt overnight. The mixture was partitioned between 1:1 EtOAc/heptane and water. The aqueous layer was extracted with 1:1 EtOAc/heptane. The combined organic layers were washed with brine, dried (Na₂SO₄) and concentrated to provide the title compound, which was used in the next reaction without further purification. MS (ESI−) m/z 466.3, 468.2, 470.2 (M−H).

### Example 82-B

### 2-(2-(3,3″-bis((S)-1-amino-2-hydroxyethyl)-[1,1′:3′,1″-terphenyl]-5′-ylcarboxamido)phenyl)acetic acid

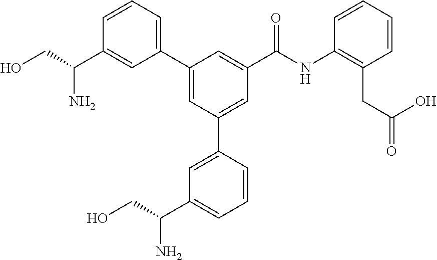 [SMILES: N[C@H](CO)c1cccc(-c2cc(C(=O)Nc3ccccc3CC(=O)O)cc(-c3cccc([C@H](N)CO)c3)c2)c1]

The title compound was synthesized from tert-butyl 2-(2-(3,5-dibromobenzamido)phenyl)acetate in a similar manner as described in Example 81. ¹H NMR (400 MHz, METHANOL-d₄) δ ppm 8.42 (d, J=1.52 Hz, 2H) 8.15 (t, J=1.64 Hz, 1H) 8.00 (d, J=7.58 Hz, 1H) 7.93 (s, 2H) 7.80 (d, J=8.21 Hz, 2H) 7.53 (t, J=7.71 Hz, 2H) 7.44 (d, J=7.70 Hz, 2H) 7.26-7.36 (m, 2H) 7.14 (td, J=7.52, 1.26 Hz, 1H) 4.26 (dd, J=8.02, 4.61 Hz, 2H) 3.83-3.91 (m, 2H) 3.75-3.83 (m, 2H) 3.58-3.70 (m, 2H). HRMS calcd. for C₃₁H₃₁N₃O₅ (M+H)⁺526.2342. found 526.2318.

### Example 83

### Example 83-A

### tert-Butyl 2-(2-(3-bromo-5-chlorobenzamido)phenyl)acetate

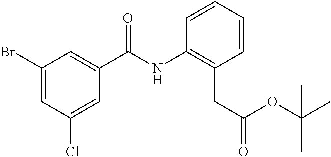 [SMILES: CC(C)(C)OC(=O)Cc1ccccc1NC(=O)c1cc(Cl)cc(Br)c1]

TEA (1.184 mL, 8.49 mmol) was added to a mixture of 3-bromo-5-chlorobenzoic acid (1 g, 4.25 mmol), tert-butyl 2-(2-aminophenyl)acetate (CAS #98911-34-3) (0.880 g, 4.25 mmol) and HATU (1.776 g, 4.67 mmol) in DMF at 23° C. The mixture was stirred at rt overnight. The mixture was partitioned between 1:1 EtOAc/heptane and water. The aqueous layer was extracted with 1:1 EtOAc/heptane. The combined organic layers were washed with brine, dried (Na₂SO₄) and concentrated. The residue was purified by flash column (EtOAc-Heptane 0-100%) to give the title compound. MS (ESI−) m/z 424.3 (M+H).

### Example 83-B

### tert-Butyl 2-(2-(3-chloro-5-((2-methoxyethyl)amino)benzamido)phenyl)acetate

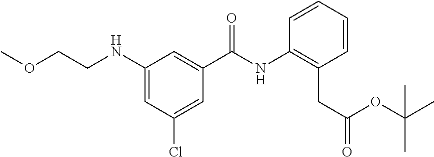 [SMILES: COCCNc1cc(Cl)cc(C(=O)Nc2ccccc2CC(=O)OC(C)(C)C)c1]

A suspension of tert-butyl 2-(2-(3-bromo-5-chlorobenzamido)phenyl)acetate (1.1 g, 2.59 mmol), 2-methoxyethanamine (CAS #109-85-3) (0.195 g, 2.59 mmol), BrettPhos palladacycle (CAS #1148148-01-9) (0.103 g, 0.129 mmol) and Cs₂CO₃ (2.53 g, 7.77 mmol) in MeCN (2 mL) was heated in a microwave at 110° C. for 60 min. the mixture was filtered and the filtrate was concentrated. The residue was purified by flash column (EtOAc-Heptane 0-100%) to provide the title compound. MS (ESI−) m/z 417.4 (M−H).

### Example 83-C

### (S)-tert-Butyl 2-(2-(3′-(1-((tert-butoxycarbonyl)amino)-2-hydroxyethyl)-5-((2-methoxyethyl)amino)-[1,1′-biphenyl]-3-ylcarboxamido)phenyl)acetate

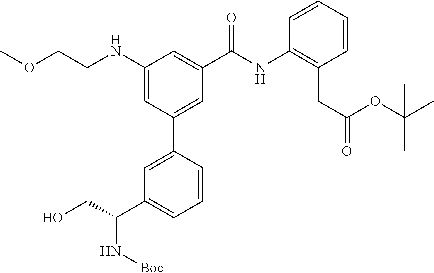 [SMILES: COCCNc1cc(C(=O)Nc2ccccc2CC(=O)OC(C)(C)C)cc(-c2cccc([C@@H](CO)NC(=O)OC(C)(C)C)c2)c1]

To a microwave vial was added tert-butyl 2-(2-(3-chloro-5-((2-methoxyethyl)amino)benzamido)phenyl)acetate (50 mg, 0.119 mmol) and (S)-tert-butyl (2-hydroxy-1-(3-(4,4,5,5-tetramethyl-1,3,2-dioxaborolan-2-yl)phenyl)ethyl)carbamate (Intermediate 34-B) (65.0 mg, 0.179 mmol) in MeCN (2 ml). At this point K₃PO₄ (2M aq. solution, 0.298 ml, 0.597 mmol) and Sphos palladacycle (CAS #1375325-64-6) (4.01 mg, 5.97 μmol) were added and the vial was sealed and heated in a microwave at 110° C. for 60 min. The reaction mixture was cooled to rt, acidified with 1N HCl solution to pH 5. The organic layer was filtered and purified by HPLC (Method B) to provide the title compound. MS (ESI−) m/z 618.7 (M−H).

### Example 83-D

### (S)-2-(2-(3′-(1-amino-2-hydroxyethyl)-5-((2-methoxyethyl)amino)-[1,1′-biphenyl]-3-ylcarboxamido)phenyl)acetic acid

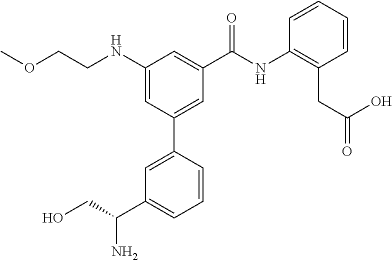 [SMILES: COCCNc1cc(C(=O)Nc2ccccc2CC(=O)O)cc(-c2cccc([C@H](N)CO)c2)c1]

A solution of (S)-tert-butyl 2-(2-(3′-(1-((tert-butoxycarbonyl)amino)-2-hydroxyethyl)-5-((2-methoxyethyl)amino)-[1,1′-biphenyl]-3-ylcarboxamido)phenyl)acetate (73.7 mg, 0.119 mmol) in DCM (1 mL) and TFA (1 mL) was stirred at 23° C. for 60 min. The reaction mixture was concentrated and the residue was purified by HPLC (Method B) to provide the title compound. ¹H NMR (400 MHz, METHANOL-d₄) δ ppm 7.92-7.98 (m, 2H) 7.70-7.75 (m, 2H) 7.50 (t, J=7.71 Hz, 1H) 7.37 (d, J=7.83 Hz, 1H) 7.24-7.34 (m, 3H) 7.08-7.14 (m, 2H) 4.39 (t, J=6.57 Hz, 1H) 3.91 (d, J=6.57 Hz, 2H) 3.58-3.67 (m, 4H) 3.37-3.43 (m, 5H). HRMS calcd. for C₂₆H₂₉N₃O₅ (M+H)⁺ 464.2185. found 464.2166.

### Example 84

### Example 84-A

### methyl 2-(2-((3-chloro-5-((2-methoxyethyl)amino)benzyl)oxy)phenyl)acetate

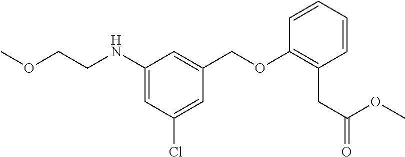

The title compound was synthesized in a similar manner as described in Example 83-B starting with methyl 2-(2-((3-bromo-5-chlorobenzyl)oxy)phenyl)acetate (Intermediate 35). MS (ESI+) m/z 364.1 (M+H).

### Example 84-B

### (S)-Methyl 2-(2-((3′-(1-((tert-butoxycarbonyl)amino)-2-hydroxyethyl)-5-((2-methoxyethyl)amino)-[1,1′-biphenyl]-3-yl)methoxy)phenyl)acetate

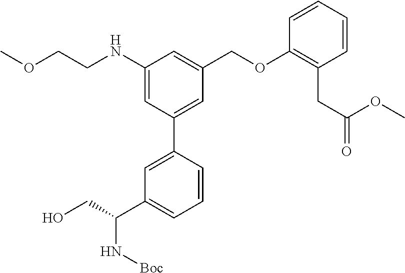

The title compound was synthesized in a similar manner as described in Example 83-C starting with methyl 2-(2-((3-chloro-5-((2-methoxyethyl)amino)benzyl)oxy)phenyl)acetate. MS (ESI+) m/z 565.2 (M+H).

### Example 84-C

### (S)-methyl 2-(2-((3′-(1-amino-2-hydroxyethyl)-5-((2-methoxyethyl)amino)-[1,1′-biphenyl]-3-yl)methoxy)phenyl)acetate

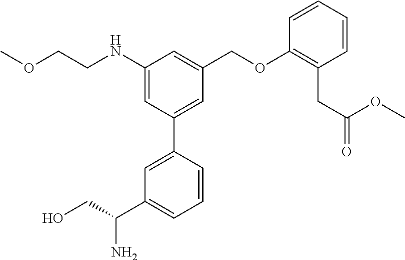

A solution of (S)-methyl 2-(2-((3′-(1-((tert-butoxycarbonyl)amino)-2-hydroxyethyl)-5-((2-methoxyethyl)amino)-[1,1′-biphenyl]-3-yl)methoxy)phenyl)acetate (135 mg, 0.24 mmol) in DCM (1 mL) and TFA (1 mL) was stirred at 23° C. for 60 min. The reaction mixture was concentrated to provide the title compound. MS (ESI+) m/z 464.9 (M+H).

### Example 84-D

### (S)-2-(2-((3′-(1-Amino-2-hydroxyethyl)-5-((2-methoxyethyl)amino)-[1,1′-biphenyl]-3-yl)methoxy)phenyl)acetic acid

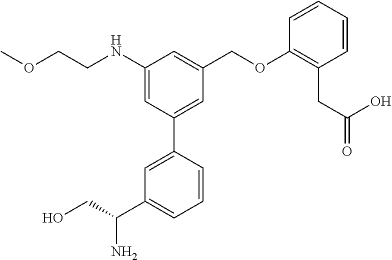 [SMILES: COCCNc1cc(COc2ccccc2CC(=O)O)cc(-c2cccc([C@H](N)CO)c2)c1]

To a solution of (S)-methyl 2-(2-((3′-(1-amino-2-hydroxyethyl)-5-((2-methoxyethyl)amino)-[1,1′-biphenyl]-3-yl)methoxy)phenyl)acetate (111 mg, 0.24 mmol) in MeOH (2 mL) was added 1M aq. LiOH (1.20 mL, 1.20 mmol). The resulting mixture was stirred at 50° C. for 60 min. Another 7 ml of methanol was added. The reaction mixture was filtered and the filtrate was purified by preparative HPLC (Method B) to provide the title compound. ¹H NMR (400 MHz, METHANOL-d₄) δ ppm 7.85 (s, 1H) 7.57 (d, J=7.83 Hz, 1H) 7.40 (t, J=7.64 Hz, 1H) 7.29 (d, J=7.58 Hz, 1H) 7.16-7.22 (m, 2H) 7.10-7.16 (m, 1H) 6.94 (d, J=7.58 Hz, 1H) 6.82-6.88 (m, 2H) 6.76 (s, 1H) 5.03-5.14 (m, 2H) 4.15 (dd, J=7.77, 5.12 Hz, 1H) 3.72-3.82 (m, 2H) 3.57-3.65 (m, 4H) 3.39 (s, 3H) 3.35 (t, J=5.49 Hz, 2H). HRMS calcd. for C₂₆H₃₀N₂O₅ (M+H)⁺ 451.2227. found 451.2162.

### Example 85

### (R)-Ethyl 2-(2-(3′-(aminomethyl)-5′-fluoro-5-(((tetrahydrofuran-2-yl)methyl)amino)-[1,1′-biphenyl]-3-ylcarboxamido)phenyl)acetate

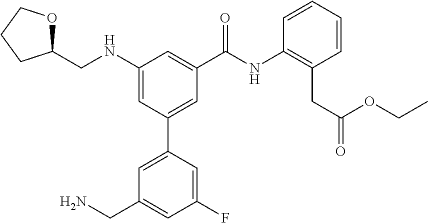 [SMILES: CCOC(=O)Cc1ccccc1NC(=O)c1cc(NC[C@H]2CCCO2)cc(-c2cc(F)cc(CN)c2)c1]

The title compound was synthesized from (R)-2-(2-(3′-(aminomethyl)-5′-fluoro-5-(((tetrahydrofuran-2-yl)methyl)amino)-[1,1′-biphenyl]-3-ylcarboxamido)phenyl)acetic acid (Example 14-D) in a similar manner as described in Example 67-C. ¹H NMR (400 MHz, METHANOL-d₄) δ ppm 7.57 (d, J=7.70 Hz, 1H) 7.49 (d, J=11.37 Hz, 2H) 7.29-7.38 (m, 3H) 7.22-7.28 (m, 2H) 7.09-7.15 (m, 2H) 4.13-4.20 (m, 1H) 4.10 (q, J=7.20 Hz, 2H) 3.87-3.95 (m, 3H) 3.75-3.82 (m, 3H) 3.33-3.39 (m, 1H, partially overlapped with solvent) 3.23-3.33 (m, 1H, partially overlapped with solvent) 2.04-2.14 (m, 1H) 1.87-2.04 (m, 2H) 1.68-1.79 (m, 1H) 1.14 (t, J=7.14 Hz, 3H). HRMS calcd. for C₂₆H₂₇N₃O₄ (M+H)⁺ 506.2449. found 506.2422.

### Example 86

### (±)-2-(2-(3′-(1-Amino-2-hydroxyethyl)-[1,1′-biphenyl]-3-ylcarboxamido)phenyl)acetic acid

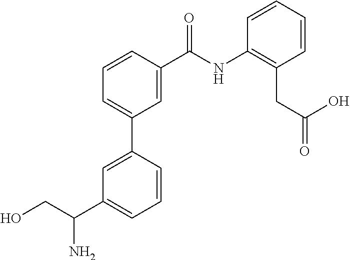 [SMILES: NC(CO)c1cccc(-c2cccc(C(=O)Nc3ccccc3CC(=O)O)c2)c1]

To a microwave vial was added methyl 2-(2-(3-(4,4,5,5-tetramethyl-1,3,2-dioxaborolan-2-yl)benzamido)phenyl)acetate (Intermediate 50) (65 mg, 0.164 mmol) and (±)-2-amino-2-(3-bromophenyl)ethanol (CAS #188586-75-6) (46.2 mg, 0.214 mmol) in 9:1 MeCN/H₂O (2 mL). A solution of 2 M aq. K₃PO₄ (0.247 mL, 0.493 mmol) and PdCl₂(dppf).CH₂Cl₂ adduct (CAS #95464-05-4) (6.71 mg, 8.22 μmol) were added and the reaction mixture was heated in a microwave at 110° C. for 90 min. The organic layer was separated and purified by HPLC (Method B) to provide the title compound. ¹H NMR (400 MHz, METHANOL-d₄) δ ppm 8.48 (s, 1H), 8.08 (d, J=6.3 Hz, 1H), 7.86-7.98 (m, 3H), 7.74 (d, J=7.3 Hz, 1H), 7.63 (t, J=7.7 Hz, 1H), 7.52 (t, J=7.7 Hz, 1H), 7.41 (d, J=7.6 Hz, 1H), 7.24-7.34 (m, 2H), 7.09-7.15 (m, 1H), 4.25-4.32 (m, 1H), 3.75-3.90 (m, 2H), 3.57-3.64 (m, 2H). HRMS calcd. for C₂₃H₂₂N₂O₄ (M+H)⁺ 391.1658. found 391.1647.

### Example 87

### (±)-2-(2-(3′-(1-amino-3-hydroxypropyl)-[1,1′-biphenyl]-3-ylcarboxamido)phenyl)acetic acid

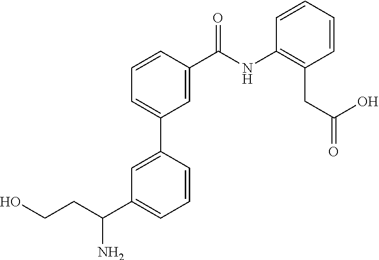 [SMILES: NC(CCO)c1cccc(-c2cccc(C(=O)Nc3ccccc3CC(=O)O)c2)c1]

The title compound was synthesized from methyl 2-(2-(3-(4,4,5,5-tetramethyl-1,3,2-dioxaborolan-2-yl)benzamido)phenyl)acetate (Intermediate 50) and (±)-3-amino-3-(3-bromophenyl)propan-1-ol hydrochloride (CAS #1379957-89-7) as described in Example 86. ¹H NMR (400 MHz, METHANOL-d4) δ ppm 8.52 (s, 1H), 8.06-8.10 (m, 1H), 7.98-8.02 (m, 1H), 7.97 (d, J=6.8 Hz, 1H), 7.91 (d, J=7.8 Hz, 1H), 7.77 (d, J=6.3 Hz, 1H), 7.64 (t, J=7.7 Hz, 1H), 7.54 (t, J=7.8 Hz, 1H), 7.42 (d, J=7.8 Hz, 1H), 7.25-7.34 (m, 2H), 7.10-7.15 (m, 1H), 4.44-4.52 (m, 1H), 3.65-3.73 (m, 1H), 3.51-3.63 (m, 3H), 2.11-2.32 (m, 2H). HRMS calcd. for C₂₄H₂₄N₂O₄ (M+H)⁺ 405.1815. found 405.1803.

### Example 88

### (R)-2-(2-(3′-(1-Amino-3-hydroxypropyl)-[1,1′-biphenyl]-3-ylcarboxamido)phenyl)acetic acid

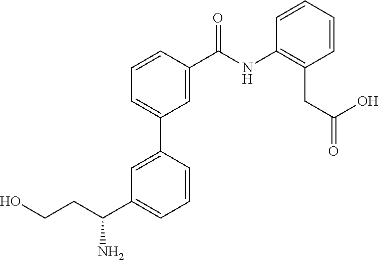 [SMILES: N[C@H](CCO)c1cccc(-c2cccc(C(=O)Nc3ccccc3CC(=O)O)c2)c1]

The title compound was synthesized from methyl 2-(2-(3-(4,4,5,5-tetramethyl-1,3,2-dioxaborolan-2-yl)benzamido)phenyl)acetate (Intermediate 50) and (R)-3-amino-3-(3-bromophenyl)propan-1-ol hydrochloride (CAS #1213637-86-5) as described in Example 86. ¹H NMR (TFA salt, 400 MHz, DMSO-d₆) δ ppm 12.37 (br. s., 1H), 10.13 (s, 1H), 8.15-8.50 (m., 4H), 7.97 (d, J=7.6 Hz, 1H), 7.92 (d, J=7.6 Hz, 1H), 7.88 (s, 1H), 7.82 (d, J=7.8 Hz, 1H), 7.67 (t, J=7.7 Hz, 1H), 7.59 (t, J=7.6 Hz, 1H), 7.48 (t, J=7.8 Hz, 2H), 7.30-7.38 (m, 2H), 7.22-7.27 (m, 1H), 4.82 (br. s., 1H), 4.47 (br. s., 1H), 3.69 (s, 2H), 3.26-3.34 (m, 1H), 2.07-2.18 (m, 1H), 1.95-2.05 (m, 1H). HRMS calcd. for C₂₄H₂₄N₂O₄ (M+H)⁺ 405.1815. found 405.1819.

### Example 89

### (S)-2-(2-(3′-(1-Amino-3-hydroxypropyl)-[1,1′-biphenyl]-3-ylcarboxamido)phenyl)acetic acid

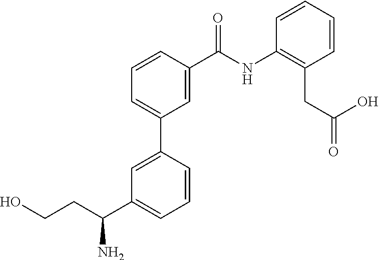 [SMILES: N[C@@H](CCO)c1cccc(-c2cccc(C(=O)Nc3ccccc3CC(=O)O)c2)c1]

The title compound was synthesized from methyl 2-(2-(3-(4,4,5,5-tetramethyl-1,3,2-dioxaborolan-2-yl)benzamido)phenyl)acetate (Intermediate 50) and (S)-3-amino-3-(3-bromophenyl)propan-1-ol hydrochloride (CAS #1213186-22-1) as described in Example 86. ¹H NMR (TFA salt, 400 MHz, DMSO-d₆) δ ppm 12.37 (br. s., 1H), 10.13 (s, 1H), 8.14-8.48 (m., 4H), 7.97 (d, J=7.8 Hz, 1H), 7.92 (d, J=8.1 Hz, 1H), 7.88 (s, 1H), 7.82 (d, J=7.8 Hz, 1H), 7.67 (t, J=7.7 Hz, 1H), 7.59 (t, J=7.7 Hz, 1H), 7.48 (t, J=7.7 Hz, 2H), 7.30-7.38 (m, 2H), 7.24 (td, J=7.3, 1.3 Hz, 1H), 4.83 (br. s., 1H), 4.44-4.53 (m, 1H), 3.69 (s, 2H), 3.25-3.34 (m, 1H), 2.07-2.20 (m, 1H), 1.93-2.06 (m, 1H). HRMS calcd. for C₂₄H₂₄N₂O₄ (M+H)⁺ 405.1815. found 405.1810.

### Example 90

### (R)-2-(2-(3′-(1-aminoethyl)-[1,1′-biphenyl]-3-ylcarboxamido)phenyl)acetic acid

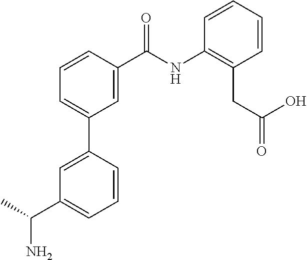 [SMILES: C[C@@H](N)c1cccc(-c2cccc(C(=O)Nc3ccccc3CC(=O)O)c2)c1]

The title compound was synthesized from methyl 2-(2-(3-(4,4,5,5-tetramethyl-1,3,2-dioxaborolan-2-yl)benzamido)phenyl)acetate (Intermediate 50) and (R)-1-(3-bromophenyl)ethanamine (CAS #176707-77-0) as described in Example 86. ¹H NMR (400 MHz, DMSO-d₆+5 μL TFA) δ ppm 10.13 (s, 1H), 8.14-8.48 (m., 4H), 7.97 (d, J=7.8 Hz, 1H), 7.86-7.94 (m, 2H), 7.80 (d, J=7.8 Hz, 1H), 7.66 (t, J=7.7 Hz, 1H), 7.59 (t, J=7.1 Hz, 1H), 7.44-7.53 (m, 2H), 7.29-7.39 (m, 2H), 7.20-7.27 (m, 1H), 4.48-4.59 (m, 1H), 3.69 (s, 2H), 1.57 (d, J=6.8 Hz, 3H). HRMS calcd. for C₂₃H₂₂N₂O₃ (M+H)⁺ 375.1709. found 375.1694.

### Example 91

### 2-(2-(3′-(Aminomethyl)-2-methoxy-[1,1′-biphenyl]-3-ylcarboxamido)phenyl)acetic acid

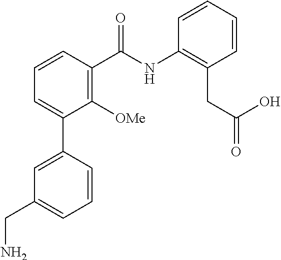 [SMILES: COc1c(C(=O)Nc2ccccc2CC(=O)O)cccc1-c1cccc(CN)c1]

The title compound was synthesized from methyl 2-(2-(3-bromo-2-methoxybenzamido)-phenyl)acetate (Intermediate 51) and (3-(aminomethyl)phenyl)boronic acid hydrochloride (CAS #146285-80-5) as described in Example 86. ¹H NMR (400 MHz, METHANOL-d₄) δ ppm 7.83-7.92 (m, 3H), 7.60 (d, J=7.6 Hz, 1H), 7.49-7.57 (m, 2H), 7.42 (d, J=7.3 Hz, 1H), 7.34 (t, J=7.6 Hz, 1H), 7.24-7.30 (m, 2H), 7.10-7.17 (m, 1H), 4.14 (br. s., 2H), 3.61 (s, 2H), 3.50 (s, 3H). HRMS calcd. for C₂₃H₂₂N₂O₄ (M+H)⁺ 391.1658. found 391.1649.

### Example 92

### 2-(2-(3′-(1-Aminocyclopropyl)-[1,1′-biphenyl]-3-ylcarboxamido)phenyl)acetic acid

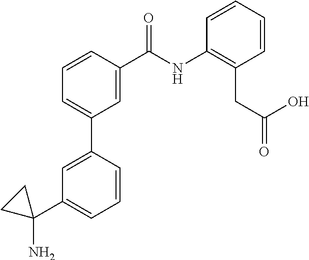 [SMILES: NC1(c2cccc(-c3cccc(C(=O)Nc4ccccc4CC(=O)O)c3)c2)CC1]

The title compound was synthesized from methyl 2-(2-(3-(4,4,5,5-tetramethyl-1,3,2-dioxaborolan-2-yl)benzamido)phenyl)acetate (Intermediate 50) and 1-(3-bromophenyl)cyclopropanamine (CAS #546115-65-5) as described in Example 86. ¹H NMR (400 MHz, DMSO-d₆) δ ppm 13.53 (br. s., 1H), 8.57 (s, 1H), 7.98-8.07 (m, 3H), 7.91 (d, J=7.6 Hz, 1H), 7.58-7.65 (m, 2H), 7.42 (t, J=7.7 Hz, 1H), 7.12-7.24 (m, 3H), 7.02 (td, J=7.3, 0.8 Hz, 1H), 3.44 (s, 2H), 1.13 (br. s., 4H). HRMS calcd. for C₂₄H₂₂N₂O₃ (M+H)⁺ 387.1709. found 387.1698.

### Example 93

### Example 93-A

### 2-(2-(3′-(((tert-Butoxycarbonyl)amino)methyl)-5′-fluoro-2-methoxy-[1,1′-biphenyl]-3-ylcarboxamido)phenyl)acetic acid

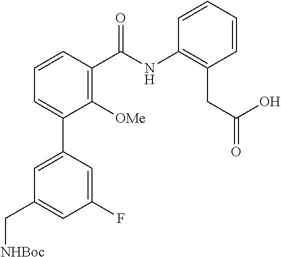 [SMILES: COc1c(C(=O)Nc2ccccc2CC(=O)O)cccc1-c1cc(F)cc(CNC(=O)OC(C)(C)C)c1]

The title compound was synthesized from methyl 2-(2-(3-bromo-2-methoxy-benzamido)phenyl)acetate (Intermediate 51) and tert-butyl 3-fluoro-5-(4,4,5,5-tetramethyl-1,3,2-dioxaborolan-2-yl)benzylcarbamate (Intermediate 24) as described in Example 86. MS (ESI+) m/z 509.5 (M+H).

### Example 93-B

### 2-(2-(3′-(Aminomethyl)-5′-fluoro-2-methoxy-[1,1′-biphenyl]-3-ylcarboxamido)phenyl)acetic acid

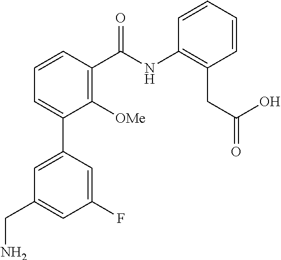 [SMILES: COc1c(C(=O)Nc2ccccc2CC(=O)O)cccc1-c1cc(F)cc(CN)c1]

In a 20 mL vial 2-(2-(3′-(((tert-butoxycarbonyl)amino)methyl)-5′-fluoro-2-methoxy-[1,1′-biphenyl]-3-ylcarboxamido)phenyl)acetic acid (32 mg, 0.063 mmol) was dissolved in THF (0.5 mL) and HCl (4M in dioxane) (0.157 mL, 0.629 mmol) was added and the reaction was stirred at room temperature for 6 h. The solvent was removed in vacuo to provide the title compound as HCl salt. ¹H NMR (HCl salt, 400 MHz, METHANOL-d₄) δ ppm 7.94 (dd, J=7.7, 1.6 Hz, 1H), 7.68 (d, J=7.8 Hz, 1H), 7.59 (dd, J=7.6, 1.8 Hz, 1H), 7.54 (s, 1H), 7.50 (d, J=9.6 Hz, 1H), 7.33-7.41 (m, 3H), 7.22-7.32 (m, 2H), 4.23 (s, 2H), 3.75 (s, 2H), 3.56 (s, 3H). HRMS calcd. for C₂₃H₂₁FN₂O₄(M+H)⁺ 409.1564. found 409.1556.

### Example 94

### Example 94-A

### tert-Butyl 2-(2-((3-chloro-5-((cyclopropylmethyl)amino)benzyl)oxy)phenyl)acetate

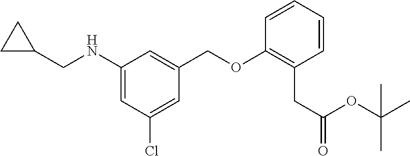 [SMILES: CC(C)(C)OC(=O)Cc1ccccc1OCc1cc(Cl)cc(NCC2CC2)c1]

The title compound was synthesized in a similar manner as described in Example 83-B starting with tert-butyl 2-(2-((3-bromo-5-chlorobenzyl)oxy)phenyl)acetate (Intermediate 53) and cyclopropylmethanamine. MS (ESI+) m/z 402.1 (M+H).

### Example 94-B

### (R)-tert-Butyl 2-(2-((3′-(1-((tert-butoxycarbonyl)amino)-3-hydroxypropyl)-5-((cyclopropylmethyl)amino)-[1,1′-biphenyl]-3-yl)methoxy)phenyl)acetate

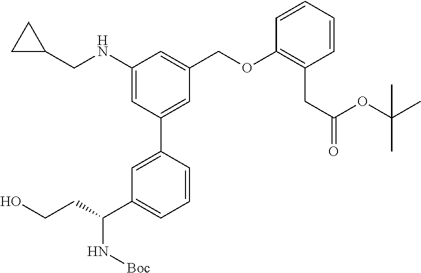 [SMILES: CC(C)(C)OC(=O)Cc1ccccc1OCc1cc(NCC2CC2)cc(-c2cccc([C@@H](CCO)NC(=O)OC(C)(C)C)c2)c1]

The title compound was synthesized in a similar manner as described in Example 83-C starting with tert-butyl 2-(2-((3-chloro-5-((cyclopropylmethyl)amino)benzyl)oxy)phenyl)acetate and (R)-tert-Butyl (3-hydroxy-1-(3-(4,4,5,5-tetramethyl-1,3,2-dioxaborolan-2-yl)phenyl)propyl)carbamate (Intermediate 30-B). MS (ESI+) m/z 617.3 (M+H).

### Example 94-C

### (R)-2-(2-((3′-(1-amino-3-hydroxypropyl)-5-((cyclopropylmethyl)amino)-[1,1′-biphenyl]-3-yl)methoxy)phenyl)acetic acid

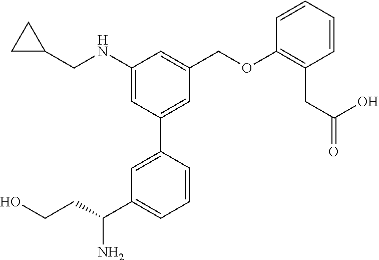 [SMILES: N[C@H](CCO)c1cccc(-c2cc(COc3ccccc3CC(=O)O)cc(NCC3CC3)c2)c1]

The title compound was synthesized in a similar manner as described in Example 83-D starting with (R)-tert-butyl 2-(2-((3′-(1-((tert-butoxycarbonyl)amino)-3-hydroxypropyl)-5-((cyclopropylmethyl)amino)-[1,1′-biphenyl]-3-yl)methoxy)phenyl)acetate. ¹H NMR (400 MHz, METHANOL-d₄) δ ppm 8.14 (s, 1H) 7.64 (d, J=8.21 Hz, 1H) 7.42 (t, J=7.64 Hz, 1H) 7.36 (s, 1H) 7.28 (d, J=7.45 Hz, 1H) 7.12-7.19 (m, 2H) 6.94 (d, J=7.71 Hz, 1H) 6.82-6.89 (m, 2H) 6.67 (s, 1H) 5.02-5.15 (m, 2H) 4.45 (t, J=7.33 Hz, 1H) 3.60-3.70 (m, 2H) 3.45-3.56 (m, 2H) 3.03 (d, J=6.69 Hz, 2H) 2.24-2.36 (m, 1H) 2.11-2.23 (m, 1H) 1.08-1.15 (m, 1H) 0.51-0.59 (m, 2H) 0.25-0.31 (m, 2H). HRMS calcd. for C₂₈H₃₂N₂O₄ (M+H)⁺ 461.2440. found 461.2434.

### Example 95

### Example 95-A

### Methyl 3-bromo-5-((2-(2-(tert-butoxy)-2-oxoethyl)phenyl)carbamoyl)benzoate

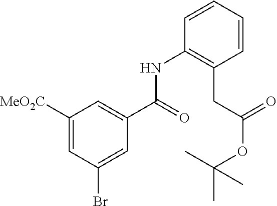 [SMILES: COC(=O)c1cc(Br)cc(C(=O)Nc2ccccc2CC(=O)OC(C)(C)C)c1]

TEA (0.726 mL, 5.21 mmol) was added to a mixture of 3-bromo-5-(methoxycarbonyl)benzoic acid (CAS #161796-10-7) (0.50 g, 1.737 mmol) and HATU (0.727 g, 1.911 mmol) in DMF (1.0 mL). After 20 min tert-butyl 2-(2-aminophenyl)acetate (0.360 g, 1.737 mmol) was added and the resulting mixture was stirred at room temperature for 18 h. The reaction was diluted with EtOAc and water. The aqueous phase was extracted twice with EtOAc. The combined organic layers were washed with brine, dried over Na₂SO₄, filtered and concentrated. The crude material was purified by flash column chromatography on silica gel (heptane/EtOAc=100:0 to 55:45) to give the title compound. ¹H NMR (400 MHz, DICHLOROMETHANE-d₂) 5 ppm 10.04 (br. s., 1H), 8.63 (s, 1H), 8.40 (dt, J=11.4, 1.6 Hz, 2H), 8.02 (d, J=8.1 Hz, 1H), 7.38-7.43 (m, 1H), 7.32 (dd, J=7.6, 1.0 Hz, 1H), 7.22 (td, J=7.6, 1.0 Hz, 1H), 3.98 (s, 3H), 3.68 (s, 2H), 1.51 (s, 9H). MS (ESI−) m/z 446.3, 448.3 (M−H).

### Example 95-B

### 3′-(Aminomethyl)-5-((2-(carboxymethyl)phenyl)carbamoyl)-5′-fluoro-[1,1′-biphenyl]-3-carboxylic acid

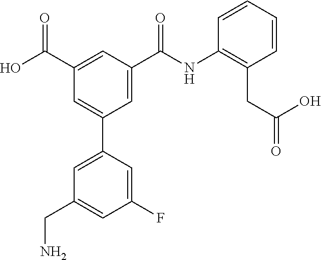 [SMILES: NCc1cc(F)cc(-c2cc(C(=O)O)cc(C(=O)Nc3ccccc3CC(=O)O)c2)c1]

In a microwave vial was placed methyl 3-bromo-5-((2-(2-(tert-butoxy)-2-oxoethyl)phenyl)carbamoyl)benzoate (0.28 g, 0.625 mmol) and tert-butyl 3-fluoro-5-(4,4,5,5-tetramethyl-1,3,2-dioxaborolan-2-yl)benzylcarbamate (Intermediate 24) (0.285 g, 0.812 mmol) in 9:1 MeCN/H₂O. 2 M aq. K₃PO₄ (0.937 mL, 1.874 mmol) and PdCl₂(dppf).CH₂Cl₂ adduct (CAS #95464-05-4) (0.026 g, 0.031 mmol) were added and the reaction mixture was heated in microwave at 110° C. for 90 min. The resulting reaction mixture was diluted with water neutralized and extracted with EtOAc. The organic phase was washed with brine, dried over Na₂SO₄ and concentrated. The crude material was stirred in DCM (1 mL) with TFA (0.5 mL) at room temperature. After 20 min the solvent was removed by rotary evaporation and the crude material purified by HPLC (Method B) to give the title compound. ¹H NMR (400 MHz, METHANOL-d₄) δ ppm 8.71 (t, J=1.6 Hz, 1H), 8.64 (t, J=1.6 Hz, 1H), 8.48 (t, J=1.6 Hz, 1H), 7.92 (d, J=8.1 Hz, 1H), 7.85 (s, 1H), 7.61 (dt, J=9.9, 1.9 Hz, 1H), 7.28-7.35 (m, 2H), 7.25 (dt, J=8.7, 2.0 Hz, 1H), 7.15 (td, J=7.6, 1.3 Hz, 1H), 4.24 (s, 2H), 3.65 (s, 2H). HRMS calcd. for C₂₃H₁₉FN₂O₅(M+H)⁺ 423.1356. found 423.1347.

### Example 96

### Example 96-A

### Methyl 3-bromo-5-propionamidobenzoate

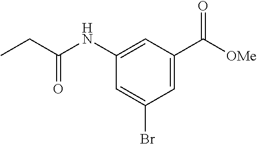 [SMILES: CCC(=O)Nc1cc(Br)cc(C(=O)OC)c1]

TEA (0.909 mL, 6.52 mmol) was added to a mixture of propionic acid (0.211 mL, 2.83 mmol) and HATU (1.157 g, 3.04 mmol) in DMF (1.0 mL). After 20 min, methyl 3-amino-5-bromobenzoate (CAS #706791-83-5) (0.5 g, 2.173 mmol) was added and the resulting mixture was stirred at room temperature for 18 h. The reaction was diluted with EtOAc and water. The aqueous phase was extracted with EtOAc. The combined organic layers were washed with brine, dried over Na₂SO₄, filtered and concentrated. The crude material was purified by flash column chromatography on silica gel (heptane/EtOAc=100:0 to 0:100) to give the title compound. ¹H NMR (400 MHz, CHLOROFORM-d) δ ppm 8.20 (s, 1H), 7.91 (app. d, J=2.0 Hz, 2H), 7.15 (br. s., 1H), 3.93 (s, 3H), 2.42 (q, J=7.5 Hz, 2H), 1.27 (t, J=7.6 Hz, 3H).

### Example 96-B

### 3-Bromo-5-propionamidobenzoic acid

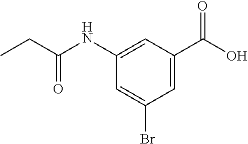

Methyl 3-bromo-5-propionamidobenzoate (0.5 g, 1.748 mmol) was dissolved in THF (5 mL). A 1M solution of LiOH (5 mL, 5.00 mmol) was added and the reaction was stirred 18 h at room temperature. The reaction mixture was concentrated in vacuo and 1N HCl was added to give a thick white precipitate which was extracted into EtOAc. The EtOAc layer was washed with brine, dried over Na₂SO₄, filtered, and evaporated to give the title compound. ¹H NMR (400 MHz, DMSO-d₆) δ ppm 13.33 (br. s., 1H), 10.23 (s, 1H), 8.17 (t, J=1.9 Hz, 1H), 8.11 (t, J=1.5 Hz, 1H), 7.68 (t, J=1.8 Hz, 1H), 2.34 (q, J=7.4 Hz, 2H), 1.08 (t, J=7.5 Hz, 3H).

### Example 96-C

### Methyl 2-(2-(3-bromo-5-propionamidobenzamido)phenyl)acetate

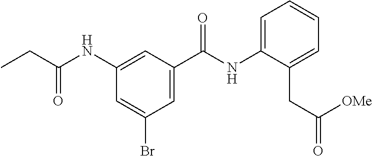 [SMILES: CCC(=O)Nc1cc(Br)cc(C(=O)Nc2ccccc2CC(=O)OC)c1]

TEA (0.231 mL, 1.654 mmol) was added to a mixture of 3-bromo-5-propionamidobenzoic acid (0.15 g, 0.551 mmol) and HATU (0.231 g, 0.606 mmol) in DMF (2 mL). After 10 min, methyl 2-(2-aminophenyl)acetate hydrochloride (CAS #49851-36-7) (0.111 g, 0.551 mmol) was added and the resulting mixture was stirred at room temperature for 48 h. The reaction was diluted with EtOAc and water. The aqueous phase was extracted with EtOAc. The combined organic layers were washed with brine, dried over Na₂SO₄, filtered and evaporated. The crude material was purified by flash column chromatography on silica gel (heptane/EtOAc=100:0 to 0:100) to give the title compound. ¹H NMR (400 MHz, DICHLOROMETHANE-d₂) δ ppm 9.49 (br. s., 1H), 8.16 (s, 1H), 7.90-7.95 (m, 2H), 7.83 (t, J=1.5 Hz, 1H), 7.33-7.39 (m, 2H), 7.29 (dd, J=7.6, 1.3 Hz, 1H), 7.18 (td, J=7.6, 1.3 Hz, 1H), 3.77 (s, 3H), 3.72 (s, 2H), 2.41 (q, J=7.6 Hz, 2H), 1.22 (t, J=7.6 Hz, 3H).

### Example 96-D

### 2-(2-(3′-(Aminomethyl)-5′-fluoro-5-propionamido-[1,1′-biphenyl]-3-ylcarboxamido)phenyl)acetic acid

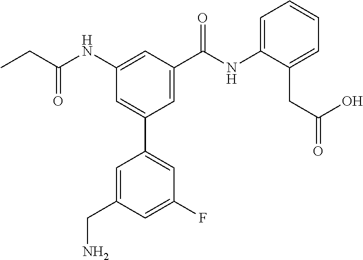 [SMILES: CCC(=O)Nc1cc(C(=O)Nc2ccccc2CC(=O)O)cc(-c2cc(F)cc(CN)c2)c1]

To a microwave vial was added methyl 2-(2-(3-bromo-5-propionamido-benzamido)phenyl)acetate (63 mg, 0.150 mmol) and tert-butyl 3-fluoro-5-(4,4,5,5-tetramethyl-1,3,2-dioxaborolan-2-yl)benzylcarbamate (Intermediate 24) (68.6 mg, 0.195 mmol) in MeCN (1 mL) with 2 M aq. K₃PO₄ (0.225 mL, 0.451 mmol) and PdCl₂(dppf).CH₂Cl₂ adduct (CAS #95464-05-4) (6.14 mg, 7.51 μmol). The reaction mixture was heated in a microwave at 110° C. for 90 min. 1N HCl was added to bring the mixture to pH 5. DCM was added and the organic phase was separated and concentrated in vacuo. DCM (1 mL) and TFA (0.5 mL) were added to the resulting residue and the reaction mixture was stirred at room temperature for 18 h. The reaction mixture was concentrated before purification by HPLC (Method B) to give the title compound. ¹H NMR (400 MHz, DMSO-d₆) δ ppm 12.71 (br. s., 1H), 10.20 (s, 1H), 8.93 (br. s., 2H), 8.36 (s, 1H), 8.29 (s, 1H), 8.11 (app. d, J=11.6 Hz, 1H), 7.98 (d, J=8.3 Hz, 1H), 7.48 (d, J=10.1 Hz, 1H), 7.31 (d, J=9.1 Hz, 1H), 7.19-7.27 (m, 2H), 7.05 (td, J=7.4, 1.1 Hz, 1H), 4.15 (s, 2H), 3.53 (s, 2H), 2.39 (q, J=7.5 Hz, 2H), 1.12 (t, J=7.6 Hz, 3H). HRMS calcd. for C₂₅H₂₄FN₃O₄(M+H)⁺ 450.1829. found 450.1810.

### Example 97

### 2-(2-(3′-(Aminomethyl)-5′-fluoro-5-isobutyramido-[1,1′-biphenyl]-3-ylcarboxamido)phenyl)acetic acid

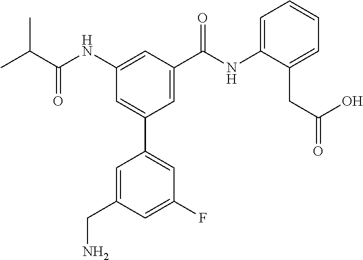 [SMILES: CC(C)C(=O)Nc1cc(C(=O)Nc2ccccc2CC(=O)O)cc(-c2cc(F)cc(CN)c2)c1]

The title compound was synthesized as in Example 96 using isobutyric acid in place of propionic acid. ¹H NMR (400 MHz, DMSO-d₆) δ ppm 12.73 (br. s., 1H), 10.17 (s, 1H), 8.89 (br. s., 2H), 8.38 (s, 1H), 8.32 (s, 1H), 8.14 (t, J=1.4 Hz, 1H), 8.10 (br. s., 1H), 7.98 (d, J=7.1 Hz, 1H), 7.45-7.54 (m, 1H), 7.31 (d, J=9.3 Hz, 1H), 7.17-7.27 (m, 2H), 7.05 (td, J=7.5, 1.0 Hz, 1H), 4.15 (s, 2H), 3.53 (s, 2H), 1.15 (d, J=6.8 Hz, 6H). HRMS calcd. for C₂₆H₂₆FN₃O₄(M+H)⁺ 464.1986. found 464.1969.

### Example 98

### 2-(2-(3′-(Aminomethyl)-5′-fluoro-5-(3-methylbutanamido)-[1,1′-biphenyl]-3-ylcarboxamido)phenyl)acetic acid

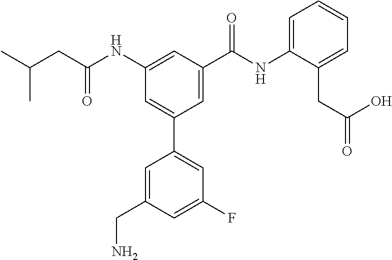 [SMILES: CC(C)CC(=O)Nc1cc(C(=O)Nc2ccccc2CC(=O)O)cc(-c2cc(F)cc(CN)c2)c1]

The title compound was synthesized as in Example 96 using 3-methylbutanoic acid in place of propionic acid. ¹H NMR (400 MHz, DMSO-d₆) δ ppm 12.73 (br. s., 1H), 10.20 (s, 1H), 8.98 (br. s, 2H), 8.37 (s, 1H), 8.27 (s, 1H), 8.15 (s, 1H), 8.10 (s, 1H), 7.98 (d, J=8.3 Hz, 1H), 7.49 (d, J=9.9 Hz, 1H), 7.31 (d, J=8.6 Hz, 1H), 7.18-7.27 (m, 2H), 7.05 (t, J=6.8 Hz, 1H), 4.15 (s, 2H), 3.53 (s, 2H), 2.25 (d, J=7.1 Hz, 2H), 2.05-2.19 (m, 1H), 0.97 (d, J=6.6 Hz, 6H). HRMS calcd. for C₂₇H₂₈FN₃O₄(M+H)⁺ 478.2142. found 478.2126.

### Example 99

### Example 99-A

### Methyl 2-(2-(chloromethyl)phenyl)acetate

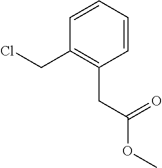

To a solution of 2-(2-(chloromethyl)phenyl)acetic acid (CAS #95335-46-9) (0.73 g, 4.0 mmol) in DCM (39.5 mL) and DMF (0.061 mL, 0.79 mmol) under nitrogen was added oxalyl chloride (0.519 mL, 5.93 mmol) and the reaction was stirred at room temperature. After 15 minutes the reaction was concentrated, then it was dissolved in DCM (39.5 mL) and methanol (1.60 mL, 39.5 mmol) was added followed by DIPEA (1.38 mL, 7.91 mmol) and the mixture was stirred at room temperature. After 30 minutes the reaction was quenched with water, extracted with DCM, dried with MgSO₄, filtered and concentrated. The crude product was purified by flash chromatography (0-40% EtOAc:Heptanes) to provide the title compound. ¹H NMR (400 MHz, DMSO-d₆) δ ppm 7.39-7.50 (m, 1H) 7.21-7.39 (m, 3H) 4.79 (s, 2H) 3.84 (s, 2H) 3.62 (s, 3H). MS (ESI+) m/z 199.1 (M+H).

### Example 99-B

### Methyl 2-(2-(((6-chloropyridin-2-yl)oxy)methyl)phenyl)acetate

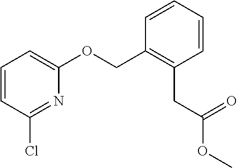 [SMILES: COC(=O)Cc1ccccc1COc1cccc(Cl)n1]

To a solution of 6-chloropyridin-2-ol (CAS #73018-09-4) (52.2 mg, 0.403 mmol) in Toluene (0.67 mL) and DMF (1.34 mL) was added K₂CO₃ (83 mg, 0.60 mmol) and the reaction was heated to 70° C. A solution of methyl 2-(2-(chloromethyl)phenyl)acetate (CAS #95360-33-1) (80 mg, 0.40 mmol) in DMF (1.34 mL) was added dropwise over 1 hour. The reaction was then heated to 80° C. After 30 minutes the reaction was cooled, diluted with water and EtOAc, extracted with EtOAc, washed with water, dried over MgSO₄, filtered and concentrated. The crude product was purified by flash chromatography (0-50% EtOAc:Heptanes) to provide the title compound. ¹H NMR (400 MHz, DMSO-d₆) δ ppm 7.77 (dd, J=8.15, 7.52 Hz, 1H) 7.43-7.52 (m, 1H) 7.24-7.37 (m, 3H) 7.11 (dd, J=7.58, 0.63 Hz, 1H) 6.75-6.85 (m, 1H) 5.31 (s, 2H) 3.82 (s, 2H) 3.58 (s, 3H). MS (ESI+) m/z 292.2 (M+H).

### Example 99-C

### 2-(2-(((6-(3-(Aminomethyl)phenyl)pyridin-2-yl)oxy)methyl)phenyl)acetic acid

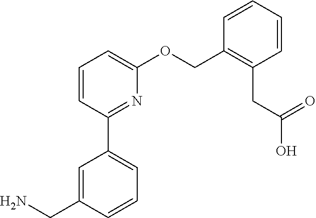 [SMILES: NCc1cccc(-c2cccc(OCc3ccccc3CC(=O)O)n2)c1]

The title compound was synthesized as described in Example 17-B starting with methyl 2-(2-(((6-chloropyridin-2-yl)oxy)methyl)phenyl)acetate, except that DMF was used instead of MeCN, and that the purification was done using preparative HPLC (Method B) to provide the title compound. ¹H NMR (400 MHz, METHANOL-d₄) δ ppm 8.20 (s, 1H) 7.88-7.98 (m, 1H) 7.72 (dd, J=8.21, 7.45 Hz, 1H) 7.38-7.52 (m, 3H) 7.32-7.38 (m, 1H) 7.25-7.32 (m, 1H) 7.11-7.25 (m, 2H) 6.80 (d, J=7.71 Hz, 1H) 5.68 (s, 2H) 3.97 (s, 2H) 3.66 (s, 2H). HRMS calcd. for C₂₁H₂₀N₂O₃ (M+H)⁺ 349.1552. found 349.1554.

### Example 100

### 2-(2-(((3′-(Aminomethyl)-[1,1′-biphenyl]-3-yl)oxy)methyl)phenyl)acetic acid

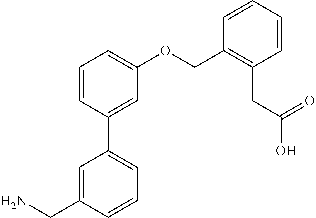 [SMILES: NCc1cccc(-c2cccc(OCc3ccccc3CC(=O)O)c2)c1]

The title compound was synthesized as described in Examples 99-B and 99-C, except using 3-bromophenol (CAS #591-20-8) in Example 99-B, and heating for 2 hours instead of 1 hour in Example 99-C. ¹H NMR (400 MHz, METHANOL-d₄) δ ppm 7.71 (s, 1H) 7.54 (d, J=7.83 Hz, 1H) 7.13-7.43 (m, 9H) 7.02 (ddd, J=8.21, 2.53, 0.88 Hz, 1H) 5.33 (s, 2H) 3.96 (s, 2H) 3.60 (s, 2H). HRMS calcd. for C₂₂H₂₁NO₃(M+H)⁺ 348.1600. found 348.1593.

### Example 101

### Example 101-A

### Methyl 2-(2-((3-bromobenzyl)oxy)phenyl)acetate

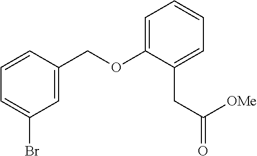 [SMILES: COC(=O)Cc1ccccc1OCc1cccc(Br)c1]

To a DMF (100 mL) suspension of methyl 2-(2-hydroxyphenyl)acetate (CAS #22446-37-3) (10 g, 60.2 mmol) and K₂CO₃ (9.56 g, 69.2 mmol) was added 3-bromobenzyl bromide (CAS #823-78-9) (16.54 g, 66.2 mmol). The mixture was stirred at room temperature for 18 hours. The mixture was diluted with EtOAc and water. The organic phase was washed four times with water, once with brine, and then dried over Na₂SO₄ before filtration and evaporation. The resulting title compound was used crude in the next step. MS (ESI+) m/z 334.9, 336.9 (M+H).

### Example 101-B

### Methyl 2-(2-((3-(4,4,5,5-tetramethyl-1,3,2-dioxaborolan-2-yl)benzyl)oxy)phenyl)acetate

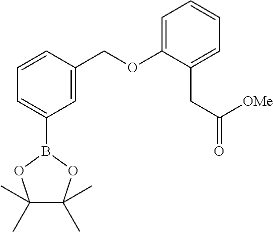 [SMILES: COC(=O)Cc1ccccc1OCc1cccc(B2OC(C)(C)C(C)(C)O2)c1]

To a solution of methyl 2-(2-((3-bromobenzyl)oxy)phenyl)acetate (21 g, 62.7 mmol) in DMF (100 mL) was added 4,4,4′,4′,5,5,5′,5′-octamethyl-2,2′-bi(1,3,2-dioxaborolane) (CAS #73183-34-3) (22.27 g, 88 mmol), potassium acetate (18.45 g, 188 mmol) and PdCl₂(dppf).CH₂Cl₂ adduct (CAS #95464-05-4) (1.834 g, 2.506 mmol). The reaction was heated at 110° C. for 90 minutes. The organic phase was washed with water, brine, and then dried over Na₂SO₄ before filtration and evaporation. The crude material was purified by flash column chromatography on silica gel (heptane/EtOAc with 10% MeOH=100:0 to 70:30) to give the title compound. MS (ESI+) m/z 383.1 (M+H).

### Example 101-C

### (S)-Methyl 2-(2-((3′-(1-((tert-butoxycarbonyl)amino)-2-hydroxyethyl)-[1,1′-biphenyl]-3-yl)methoxy)phenyl)acetate

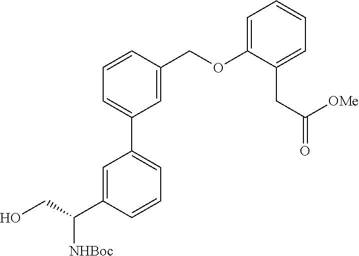 [SMILES: COC(=O)Cc1ccccc1OCc1cccc(-c2cccc([C@@H](CO)NC(=O)OC(C)(C)C)c2)c1]

A mixture of methyl 2-(2-((3-(4,4,5,5-tetramethyl-1,3,2-dioxaborolan-2-yl)benzyl)oxy)phenyl)acetate (3.04 g, 6.36 mmol) and (S)-tert-butyl (1-(3-bromophenyl)-2-hydroxyethyl)carbamate (Intermediate 34-A) (2.112 g, 6.68 mmol) in CH₃CN (30 mL)/water (15 mL) with solid K₃PO₄ (6.75 g, 31.8 mmol) and PdCl₂(dppf).CH₂Cl₂ adduct (CAS #95464-05-4) (0.206 g, 0.282 mmol) was heated to 90° C. for 1 hour and then diluted with EtOAc and saturated NH₄Cl. The layers were separated and the aqueous phase was extracted with EtOAc. The combined organic phase was washed with water and brine and then dried over Na₂SO₄. The crude material was purified by flash column chromatography on silica gel (heptane/EtOAc with 10% MeOH=100:0 to 60:40) to give the title compound. ¹H NMR (400 MHz, DMSO-d₆) 5 ppm 7.70 (s, 1H), 7.56-7.67 (m, 2H), 7.44-7.56 (m, 2H), 7.37-7.44 (m, 2H), 7.18-7.33 (m, 4H) 7.09 (d, J=8.2 Hz, 1H), 6.91 (t, J=7.4 Hz, 1H), 5.18 (s, 2H), 4.79 (t, J=5.6 Hz, 1H), 4.57-4.63 (m, 1H), 3.66 (s, 2H), 3.54 (t, J=6.2 Hz, 2H), 3.48 (s, 3H), 1.3 (s, 9H).

### Example 101-D

### (S)-2-(2-((3′-(1-((tert-Butoxycarbonyl)amino)-2-hydroxyethyl)-[1,1′-biphenyl]-3-yl)methoxy)phenyl)acetic acid

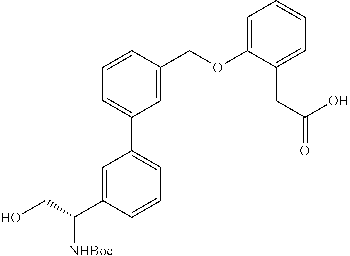 [SMILES: CC(C)(C)OC(=O)N[C@H](CO)c1cccc(-c2cccc(COc3ccccc3CC(=O)O)c2)c1]

To a mixture of (S)-methyl 2-(2-((3′-(1-((tert-butoxycarbonyl)amino)-2-hydroxyethyl)-[1,1′-biphenyl]-3-yl)methoxy)phenyl)acetate (2.1 g, 4.27 mmol) in THF (5 mL), MeOH (5 mL) and water (10 mL) and LiOH (0.51 g, 21.36 mmol) were added and the reaction was stirred at room temperature for 72 hours. The reaction mixture was acidified to pH=6 with 1N HCl and 10% citric acid and then stirred with EtOAc. The layers were separated and the aqueous phase was extracted with EtOAc. The combined organic phase was washed with brine and dried over Na₂SO₄. The crude material was purified by flash column chromatography on silica gel (heptane/EtOAc with 10% MeOH=95:5 to 50:50) to give the title compound. MS (ESI−) m/z 476.2 (M−H).

### Example 101-E

### (S)-2-(2-((3′-(1-Amino-2-hydroxyethyl)-[1,1′-biphenyl]-3-yl)methoxy)phenyl)acetic acid

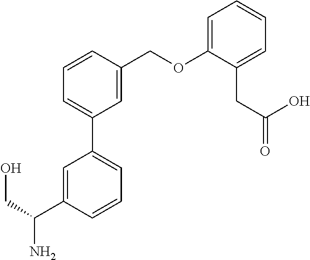 [SMILES: N[C@H](CO)c1cccc(-c2cccc(COc3ccccc3CC(=O)O)c2)c1]

(S)-2-(2-((3′-(1-((tert-Butoxycarbonyl)amino)-2-hydroxyethyl)-[1,1′-biphenyl]-3-yl)methoxy)phenyl)acetic acid (1.70 g, 3.56 mmol) was stirred in a mixture of Et₂O (60 mL), DCM (60 mL), and HCl (2M in Et₂O, 26.7 mL, 53.4 mmol) at room temperature for 72 hours. The resulting precipitate was filtered and washed with Et₂O to provide the title compound as HCl salt. ¹H NMR (HCl salt, 400 MHz, DMSO-d₆) δ ppm 12.18 (br. s., 1H), 8.36 (br. s., 3H), 7.83 (s, 1H), 7.78 (s, 1H), 7.72 (d, J=8.1 Hz, 1H), 7.64 (d, J=7.5 Hz, 1H), 7.43-7.56 (m, 4H), 7.23 (d, J=7.5 Hz, 2H), 7.06 (d, J=8.1 Hz, 1H), 6.91 (dt, J=0.7, 7.4 Hz, 1H), 5.55-5.61 (m, 1H), 5.20 (s, 2H), 4.38 (br. s., 1H), 3.69-3.82 (m, 2H), 3.60 (s, 2H). HRMS calcd. for C₂₃H₂₃NO₄ (M+H)⁺ 378.1706. found 378.1721.

### Example 102

### Example 102-A

### tert-Butyl 2-(2-((3′-(((tert-butoxycarbonyl)amino)methyl)-5-chloro-[1,1′-biphenyl]-3-yl)methoxy)phenyl)acetate

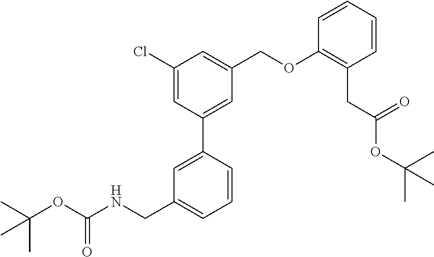 [SMILES: CC(C)(C)OC(=O)Cc1ccccc1OCc1cc(Cl)cc(-c2cccc(CNC(=O)OC(C)(C)C)c2)c1]

To a solution of tert-butyl 2-(2-((3-bromo-5-chlorobenzyl)oxy)phenyl)acetate (Intermediate 53) (2.5 g, 6.1 mmol) and (3-(((tert-butoxycarbonyl)amino)meth yl)boronic acid (CAS #199609-62-6) (1.91 g, 7.59 mmol) in acetonitrile (55.2 mL) and water (5.52 mL) under nitrogen was added 2M aqueous K₃PO₄ (9.11 mL, 18.2 mmol) and PdCl₂(dppf).CH₂Cl₂ adduct (CAS #95464-05-4) (0.248 g, 0.304 mmol) and the was stirred at 110° C. After 30 minutes the reaction was cooled to room temperature, then diluted with water and EtOAc, extracted with EtOAc, dried with MgSO₄, filtered and concentrated. This was purified by flash chromatography (0-50% EtOAc:Heptanes) to provide the title compound. MS (ESI−) m/z 536.3 (M−H).

### Example 102-B

### 2-(2-((3′-(Aminomethyl)-5-chloro-[1,1′-biphenyl]-3-yl)methoxy)phenyl)acetic acid

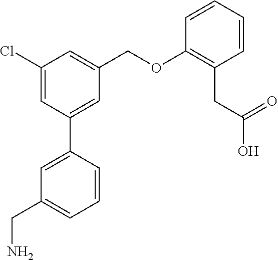 [SMILES: NCc1cccc(-c2cc(Cl)cc(COc3ccccc3CC(=O)O)c2)c1]

The title compound was synthesized as described in Example 20-B starting with tert-butyl 2-(2-((3′-(((tert-butoxycarbonyl)amino)methyl)-5-chloro-[1,1′-biphenyl]-3-yl)methoxy)phenyl)acetate. ¹H NMR (400 MHz, METHANOL-d₄) δ ppm 8.16 (s, 1H) 8.01 (s, 1H) 7.68 (d, J=8.21 Hz, 1H) 7.61 (t, J=1.77 Hz, 1H) 7.46 (t, J=7.64 Hz, 1H) 7.31-7.41 (m, 2H) 7.11-7.23 (m, 2H) 6.94 (d, J=7.83 Hz, 1H) 6.88 (td, J=7.39, 1.01 Hz, 1H) 5.20 (s, 2H) 4.18 (s, 2H) 3.60 (s, 2H). HRMS calcd. for C₂₂H₂₀ClNO₃ (M+H)⁺ 382.1210. found 382.1205.

### Example 103

### Example 103A

### tert-Butyl 2-(2-((3′-(((tert-butoxycarbonyl)amino)methyl)-5-cyclopropyl-[1,1′-biphenyl]-3-yl)methoxy)phenyl)acetate

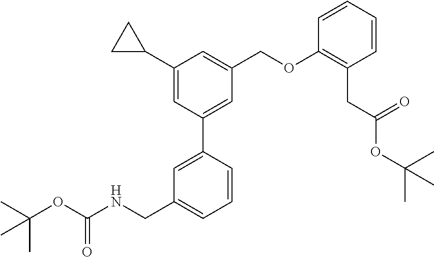 [SMILES: CC(C)(C)OC(=O)Cc1ccccc1OCc1cc(-c2cccc(CNC(=O)OC(C)(C)C)c2)cc(C2CC2)c1]

In a microwave vial, to a solution of tert-butyl 2-(2-((3′-(((tert-butoxycarbonyl)amino)methyl)-5-chloro-[1,1′-biphenyl]-3-yl)methoxy)phenyl)acetate (Example 102-A) (120 mg, 0.223 mmol) and potassium cyclopropyltrifluoroborate (CAS #1065010-87-8) (66.0 mg, 0.446 mmol) in DME (1.67 mL) was added H₂O (0.558 mL) and K₂CO₃ (61.6 mg, 0.446 mmol) and finally S-Phos palladacycle (CAS 1028206-58-7) (30.0 mg, 0.045 mmol). The reaction was heated in the microwave at 140° C. for 1 hour. The reaction was then diluted with water and EtOAc, extracted with EtOAc, dried with MgSO₄, filtered and concentrated. The crude product was purified by flash chromatography (0-40% EtOAc:Heptanes) to provide the title compound. MS (ESI+) m/z 544.5 (M+H).

### Example 103-B

### 2-(2-((3′-(Aminomethyl)-5-cyclopropyl-[1,1′-biphenyl]-3-yl)methoxy)phenyl)acetic acid

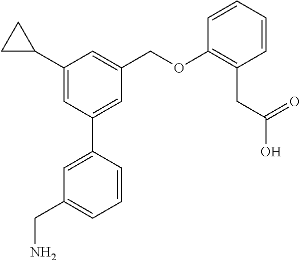 [SMILES: NCc1cccc(-c2cc(COc3ccccc3CC(=O)O)cc(C3CC3)c2)c1]

The title compound was synthesized as described in Example 20-B starting with tert-butyl 2-(2-((3′-(((tert-butoxycarbonyl)amino)methyl)-5-cyclopropyl-[1,1′-biphenyl]-3-yl)methoxy)phenyl)acetate. ¹H NMR (400 MHz, METHANOL-d₄) δ ppm 8.12 (s, 1H) 7.84 (s, 1H) 7.67 (d, J=7.83 Hz, 1H) 7.43 (t, J=7.64 Hz, 1H) 7.25-7.35 (m, 2H) 7.11-7.24 (m, 2H) 7.04 (s, 1H) 6.94 (d, J=7.96 Hz, 1H) 6.86 (t, J=7.20 Hz, 1H) 5.16 (s, 2H) 4.16 (s, 2H) 3.60 (s, 2H) 1.92-2.08 (m, 1H) 0.93-1.07 (m, 2H) 0.69-0.82 (m, 2H). HRMS calcd. for C₂₅H₂₅NO₃ (M+H)⁺ 388.1913. found 388.1905.

### Example 104

### 2-(2-((3′-(aminomethyl)-5-ethyl-[1,1′-biphenyl]-3-yl)methoxy)phenyl)acetic acid

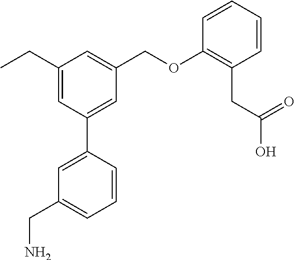 [SMILES: CCc1cc(COc2ccccc2CC(=O)O)cc(-c2cccc(CN)c2)c1]

The title compound was synthesized as described in Example 103 using potassium ethyltrifluoroborate (CAS #44248-07-9) in place of potassium cyclopropyltrifluoroborate, and heating at 140° C. for 2 hours (in Example 103-A). ¹H NMR (400 MHz, METHANOL-d₄) δ ppm 8.14 (s, 1H) 7.89 (s, 1H) 7.68 (d, J=7.70 Hz, 1H) 7.37-7.49 (m, 2H) 7.30 (d, J=7.58 Hz, 1H) 7.11-7.23 (m, 3H) 6.95 (d, J=7.58 Hz, 1H) 6.87 (td, J=7.36, 1.07 Hz, 1H) 5.18 (s, 2H) 4.16 (s, 2H) 3.60 (s, 2H) 2.74 (q, J=7.58 Hz, 2H) 1.30 (t, J=7.64 Hz, 3H). HRMS calcd. for C₂₄H₂₅NO₃ (M+H)⁺ 376.1913. found 376.1908.

### Example 105

### Example 105-A

### tert-Butyl 2-(2-((3′-(((tert-butoxycarbonyl)amino)methyl)-5-vinyl-[1,1′-biphenyl]-3-yl)methoxy)phenyl)acetate

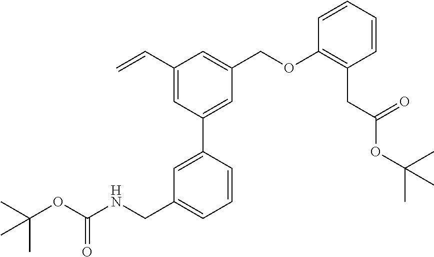 [SMILES: C=Cc1cc(COc2ccccc2CC(=O)OC(C)(C)C)cc(-c2cccc(CNC(=O)OC(C)(C)C)c2)c1]

The title compound was synthesized as described in Example 103-A starting with vinylboronic anhydride pyridine complex (CAS #442850-89-7). MS (ESI−) m/z 528.3 (M−H).

### Example 105-B

### tert-Butyl 2-(2-((3′-(((tert-butoxycarbonyl)amino)methyl)-5-(2-hydroxyethyl)-[1,1′-biphenyl]-3-yl)methoxy)phenyl)acetate

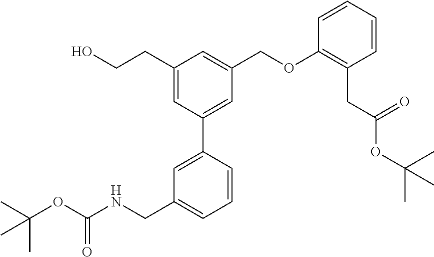 [SMILES: CC(C)(C)OC(=O)Cc1ccccc1OCc1cc(CCO)cc(-c2cccc(CNC(=O)OC(C)(C)C)c2)c1]

A solution of tert-butyl 2-(2-((3′-(((tert-butoxycarbonyl)amino)methyl)-5-vinyl-[1,1′-biphenyl]-3-yl)methoxy)phenyl)acetate (508 mg, 0.959 mmol) in THF (9.59 mL) was cooled to 0° C. under nitrogen and 9-BBN (0.5M in THF, 5.75 mL, 2.88 mmol) was added dropwise. The reaction was warmed to room temperature and stirred overnight. The mixture was then cooled again to 0° C. and NaOH (2.0M aqueous, 2.88 mL, 5.75 mmol) and H₂O₂ (50% aqueous, 0.353 mL, 5.75 mmol) were added, and then warmed again to room temperature. After 15 minutes the reaction mixture was diluted with saturated aqueous sodium thiosulfate and EtOAc, layers were separated and the aq. layer was extracted with EtOAc. The combined organics were dried over MgSO₄, filtered and concentrated. The crude product was purified by flash chromatography (0-80% EtOAc:Heptanes) to provide the title compound. MS (ESI−) m/z 546.3 (M−H).

### Example 105-C

### 2-(2-((3′-(Aminomethyl)-5-(2-hydroxyethyl)-[1,1′-biphenyl]-3-yl)methoxy)phenyl)acetic acid

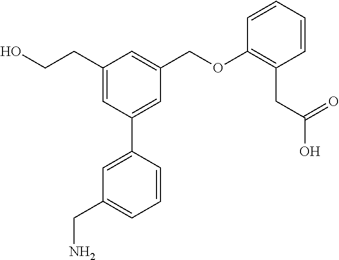 [SMILES: NCc1cccc(-c2cc(CCO)cc(COc3ccccc3CC(=O)O)c2)c1]

The title compound was synthesized as described in Example 20-B starting with tert-butyl 2-(2-((3′-(((tert-butoxycarbonyl)amino)methyl)-5-(2-hydroxyethyl)-[1,1′-biphenyl]-3-yl)methoxy)phenyl)acetate. ¹H NMR (400 MHz, METHANOL-d₄) δ ppm 8.14 (s, 1H) 7.92 (s, 1H) 7.70 (d, J=7.96 Hz, 1H) 7.48 (s, 1H) 7.43 (t, J=7.71 Hz, 1H) 7.30 (d, J=7.96 Hz, 1H) 7.23 (s, 1H) 7.11-7.20 (m, 2H) 6.95 (d, J=7.83 Hz, 1H) 6.87 (td, J=7.39, 1.01 Hz, 1H) 5.19 (s, 2H) 4.16 (s, 2H) 3.83 (t, J=6.95 Hz, 2H) 3.60 (s, 2H) 2.91 (t, J=6.95 Hz, 2H). HRMS calcd. for C₂₄H₂₅NO₄ (M+H)⁺ 392.1862. found 392.1850.

### Example 106

### (±)-2-(2-((3′-(Aminomethyl)-5-(1-hydroxyethyl)-[1,1′-biphenyl]-3-yl)methoxy)phenyl)acetic acid

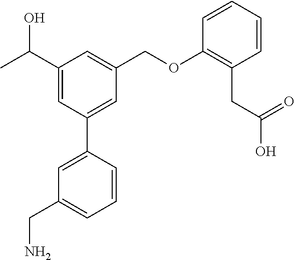 [SMILES: CC(O)c1cc(COc2ccccc2CC(=O)O)cc(-c2cccc(CN)c2)c1]

To a solution of tert-butyl 2-(2-((3′-(((tert-butoxycarbonyl)amino)methyl)-5-vinyl-[1,1′-biphenyl]-3-yl)methoxy)phenyl)acetate (Example 105-A) (72 mg, 0.136 mmol) in dioxane (0.906 mL) and Water (0.453 mL) was added H₂SO₄ (0.145 mL, 2.72 mmol) and the reaction was stirred at 60° C. for 1 day and then at 80° C. for further 24 h. The reaction was quenched with NH₄OH (0.706 mL, 5.44 mmol), and the organic layer was separated purified directly by preparative HPLC (Method B) to provide the title compound. ¹H NMR (400 MHz, METHANOL-d₄) δ ppm 8.15 (s, 1H) 7.97 (s, 1H) 7.71 (d, J=8.08 Hz, 1H) 7.62 (s, 1H) 7.44 (t, J=7.71 Hz, 1H) 7.26-7.39 (m, 2H) 7.09-7.24 (m, 2H) 6.95 (d, J=7.58 Hz, 1H) 6.87 (td, J=7.39, 1.01 Hz, 1H) 5.21 (s, 2H) 4.92 (q, J=6.44 Hz, 1H) 4.17 (s, 2H) 3.60 (s, 2H) 1.50 (d, J=6.57 Hz, 3H). HRMS calcd. for C₂₄H₂₅NO₄ (M+H)⁺ 392.1862. found 392.1855.

### Example 107

### Example 107-A

### tert-Butyl 2-(2-((3′-(((tert-butoxycarbonyl)amino)methyl)-5-(2-iodoethyl)-[1,1′-biphenyl]-3-yl)methoxy)phenyl)acetate

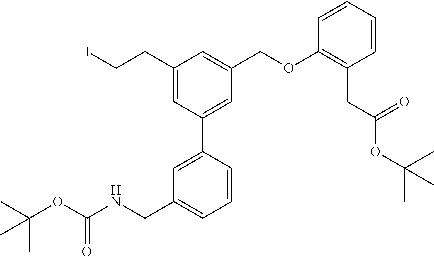 [SMILES: CC(C)(C)OC(=O)Cc1ccccc1OCc1cc(CCI)cc(-c2cccc(CNC(=O)OC(C)(C)C)c2)c1]

A solution of PPh₃ (59.9 mg, 0.228 mmol) and imidazole (15.5 mg, 0.228 mmol) in DCM (1.83 mL) was cooled to 0° C. under nitrogen, and iodine (57.9 mg, 0.228 mmol) was added. The reaction was stirred for 25 minutes. A solution of tert-butyl 2-(2-((3′-(((tert-butoxycarbonyl)amino)methyl)-5-(2-hydroxyethyl)-[1,1′-biphenyl]-3-yl)methoxy)phenyl)acetate (Example 105-B) (100 mg, 0.183 mmol) in DCM (0.9 mL) was added and the mixture was warmed to room temperature and stirred overnight. The reaction was filtered through Celite®, rinsing with DCM. The filtrate was diluted with saturated aqueous sodium thiosulfate and EtOAc, extracted with EtOAc, dried over MgSO₄, filtered and concentrated. The crude product was purified by flash chromatography (0-80% EtOAc:Heptanes) to provide the title compound. MS (ESI+) m/z 658.2 (M+H), 680.2 (M+Na).

### Example 107B

### tert-Butyl 2-(2-((3′-(((tert-butoxycarbonyl)amino)methyl)-5-(2-cyclopropylethyl)-[1,1′-biphenyl]-3-yl)methoxy)phenyl)acetate

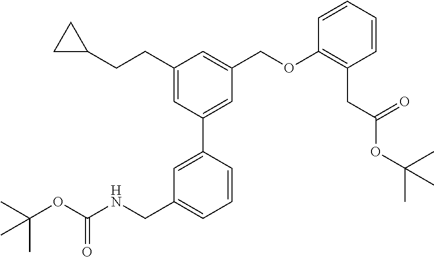 [SMILES: CC(C)(C)OC(=O)Cc1ccccc1OCc1cc(CCC2CC2)cc(-c2cccc(CNC(=O)OC(C)(C)C)c2)c1]

To a solution of tert-butyl 2-(2-((3′-(((tert-butoxycarbonyl)amino)methyl)-5-(2-iodoethyl)-[1,1′-biphenyl]-3-yl)methoxy)phenyl)acetate (66 mg, 0.10 mmol) and CuCl (1.0 mg, 0.010 mmol) in THF (0.50 mL) under nitrogen was added cyclopropylmagnesium bromide (0.5M in THF, 0.442 mL, 0.221 mmol) and the reaction was stirred at room temperature. After 20 minutes additional cyclopropylmagnesium bromide (0.5M in THF, 0.442 mL, 0.221 mmol) was added. After 40 minutes additional cyclopropylmagnesium bromide (0.5M in THF, 0.442 mL, 0.221 mmol) was added and the resulting mixture was stirred overnight. The reaction was quenched with saturated aqueous NH₄Cl, extracted with EtOAc, dried over MgSO₄, filtered and concentrated. It was purified by flash chromatography (0-40% EtOAc:Heptanes) to provide the title compound. MS (ESI+) m/z 572.4 (M+H).

### Example 107-C

### 2-(2-((3′-(Aminomethyl)-5-(2-cyclopropylethyl)-[1,1′-biphenyl]-3-yl)methoxy)phenyl)acetic acid

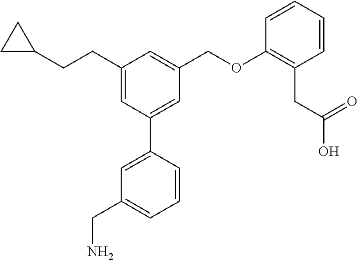 [SMILES: NCc1cccc(-c2cc(CCC3CC3)cc(COc3ccccc3CC(=O)O)c2)c1]

The title compound was synthesized as described in Example 20-B starting with tert-butyl 2-(2-((3′-(((tert-butoxycarbonyl)amino)methyl)-5-(2-cyclopropylethyl)-[1,1′-biphenyl]-3-yl)methoxy)phenyl)acetate. ¹H NMR (400 MHz, METHANOL-d₄) δ ppm 8.14 (s, 1H) 7.89 (s, 1H) 7.67 (d, J=8.08 Hz, 1H) 7.37-7.48 (m, 2H) 7.29 (d, J=8.08 Hz, 1H) 7.10-7.24 (m, 3H) 6.94 (d, J=8.08 Hz, 1H) 6.86 (t, J=7.83 Hz, 1H) 5.18 (s, 2H) 4.17 (s, 2H) 3.60 (s, 2H) 2.76-2.86 (m, 2H) 1.48-1.67 (m, 2H) 0.68-0.81 (m, 1H) 0.37-0.50 (m, 2H) 0.04-0.12 (m, 2H). HRMS calcd. for C₂₇H₂₉NO₃ (M+H)⁺ 416.2226. found 416.2219.

### Example 108

### Example 108-A

### (S)-tert-butyl 2-(2-((3′-(1-((tert-butoxycarbonyl)amino)-2-hydroxyethyl)-5-chloro-[1,1′-biphenyl]-3-yl)methoxy)phenyl)acetate

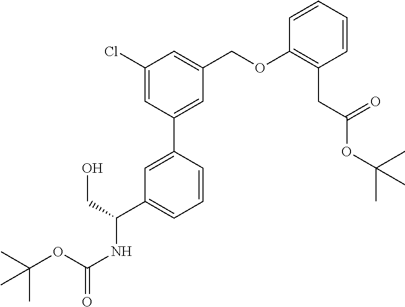 [SMILES: CC(C)(C)OC(=O)Cc1ccccc1OCc1cc(Cl)cc(-c2cccc([C@@H](CO)NC(=O)OC(C)(C)C)c2)c1]

In a microwave vial, to a mixture of tert-butyl 2-(2-((3-bromo-5-chlorobenzyl)oxy)phenyl)acetate (Intermediate 53) (0.50 g, 1.2 mmol) and (S)-tert-butyl (2-hydroxy-1-(3-(4,4,5,5-tetramethyl-1,3,2-dioxaborolan-2-yl)phenyl)ethyl)carbamate (Intermediate 34-B) (0.545 g, 1.27 mmol) in acetonitrile (7.20 mL) and water (0.900 mL) was added 2M aqueous K₃PO₄ (1.21 mL, 2.43 mmol) and PdCl₂(dppf).CH₂Cl₂ adduct (CAS #95464-05-4) (0.040 g, 0.049 mmol) and the resulting mixture was heated in a microwave at 110° C. for 60 minutes. The reaction was diluted with water and EtOAc, extracted with EtOAc, dried over MgSO₄, filtered and concentrated. The crude product was purified by flash chromatography (0-50% EtOAc:Heptanes) to provide the title compound. MS (ESI+) m/z 568.2 (M+H).

### Example 108-B

### (S)-2-(2-((3′-(1-Amino-2-hydroxyethyl)-5-chloro-[1,1′-biphenyl]-3-yl)methoxy)phenyl)acetic acid

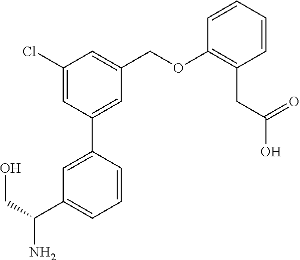 [SMILES: N[C@H](CO)c1cccc(-c2cc(Cl)cc(COc3ccccc3CC(=O)O)c2)c1]

To a solution of (S)-tert-butyl 2-(2-((3′-(1-((tert-butoxycarbonyl)amino)-2-hydroxyethyl)-5-chloro-[1,1′-biphenyl]-3-yl)methoxy)phenyl)acetate (65 mg, 0.114 mmol) in dichloromethane (1.0 mL) was added 4.0M HCl in dioxane (5.0 mL). This mixture was stirred 8 hours and then concentrated in vacuo. The resulting residue was purified by preparative HPLC (Method B) to afford the title compound. ¹H NMR (400 MHz, METHANOL-d₄) δ ppm 8.12-8.27 (m, 1H) 8.01 (s, 1H) 7.59-7.78 (m, 2H) 7.44-7.53 (m, 1H) 7.31-7.43 (m, 2H) 7.13-7.23 (m, 2H) 6.83-6.98 (m, 2H) 5.09-5.26 (m, 2H) 4.36 (dd, J=8.78, 4.61 Hz, 1H) 3.80-4.02 (m, 2H) 3.50-3.68 (m, 2H). HRMS calcd. for C₂₃H₂₂ClNO₄ (M+H)⁺ 412.1316. found 412.1331.

### Example 109

### Example 109-A

### (S)-tert-Butyl 2-(2-((3′-(1-((tert-butoxycarbonyl)amino)-2-hydroxyethyl)-5-cyclopropyl-[1,1′-biphenyl]-3-yl)methoxy)phenyl)acetate

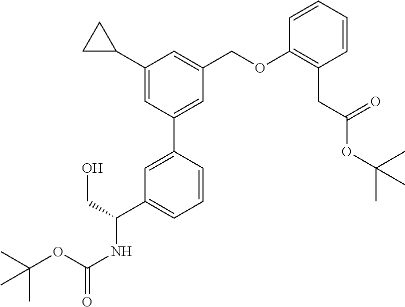 [SMILES: CC(C)(C)OC(=O)Cc1ccccc1OCc1cc(-c2cccc([C@@H](CO)NC(=O)OC(C)(C)C)c2)cc(C2CC2)c1]

The title compound was synthesized as described in Example 103-A starting with (S)-tert-butyl 2-(2-((3′-(1-((tert-butoxycarbonyl)amino)-2-hydroxyethyl)-5-chloro-[1,1′-biphenyl]-3-yl)methoxy)phenyl)acetate (Example 108-A). MS (ESI+) m/z 574.4 (M+H).

### Example 109-B

### (S)-2-(2-((3′-(1-Amino-2-hydroxyethyl)-5-cyclopropyl-[1,1′-biphenyl]-3-yl)methoxy)phenyl)acetic acid

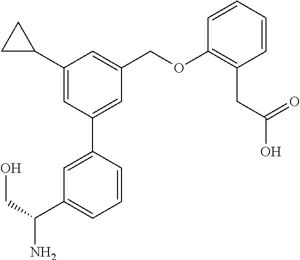 [SMILES: N[C@H](CO)c1cccc(-c2cc(COc3ccccc3CC(=O)O)cc(C3CC3)c2)c1]

The title compound was synthesized as described in Example 20-B starting with (S)-tert-butyl 2-(2-((3′-(1-((tert-butoxycarbonyl)amino)-2-hydroxyethyl)-5-cyclopropyl-[1,1′-biphenyl]-3-yl)methoxy)phenyl)acetate. ¹H NMR (400 MHz, METHANOL-d₄) δ ppm 8.14 (s, 1H) 7.84 (s, 1H) 7.68 (d, J=8.08 Hz, 1H) 7.45 (t, J=7.71 Hz, 1H) 7.28-7.35 (m, 2H) 7.11-7.20 (m, 2H) 7.05 (s, 1H) 6.95 (d, J=7.83 Hz, 1H) 6.86 (td, J=7.39, 1.01 Hz, 1H) 5.06-5.22 (m, 2H) 4.33 (dd, J=8.84, 4.55 Hz, 1H) 3.94 (dd, J=11.68, 8.91 Hz, 1H) 3.85 (dd, J=11.75, 4.67 Hz, 1H) 3.50-3.68 (m, 2H) 1.92-2.09 (m, 1H) 0.94-1.06 (m, 2H) 0.70-0.82 (m, 2H). HRMS calcd. for C₂₆H₂₇NO₄ (M+H)⁺ 418.2018. found 418.2005.

### Example 110

### Example 110-A

### (S)-tert-Butyl 2-(2-((3′-(1-((tert-butoxycarbonyl)amino)-2-hydroxyethyl)-5-vinyl-[1,1′-biphenyl]-3-yl)methoxy)phenyl)acetate

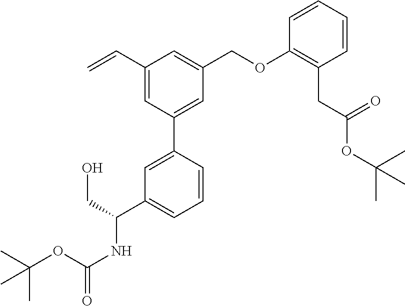 [SMILES: C=Cc1cc(COc2ccccc2CC(=O)OC(C)(C)C)cc(-c2cccc([C@@H](CO)NC(=O)OC(C)(C)C)c2)c1]

The title compound was synthesized as described in Example 105-A starting with (S)-tert-butyl 2-(2-((3′-(1-((tert-butoxycarbonyl)amino)-2-hydroxyethyl)-5-chloro-[1,1′-biphenyl]-3-yl)methoxy)phenyl)acetate (Example 108-A). MS (ESI+) m/z 560.3 (M+H).

### Example 110-B

### (S)-tert-Butyl 2-(2-((3′-(1-((tert-butoxycarbonyl)amino)-2-hydroxyethyl)-5-ethyl-[1,1′-biphenyl]-3-yl)methoxy)phenyl)acetate

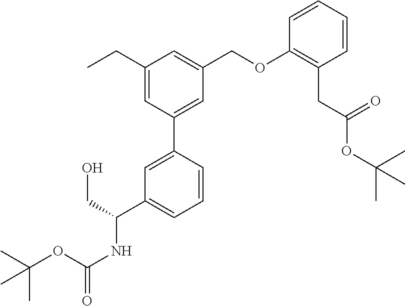 [SMILES: CCc1cc(COc2ccccc2CC(=O)OC(C)(C)C)cc(-c2cccc([C@@H](CO)NC(=O)OC(C)(C)C)c2)c1]

The title compound was synthesized as described in Intermediate 1-B (using EtOAc instead of EtOH as the solvent), starting with (S)-tert-butyl 2-(2-((3′-(1-((tert-butoxycarbonyl)amino)-2-hydroxyethyl)-5-vinyl-[1,1′-biphenyl]-3-yl)methoxy)phenyl)acetate. MS (ESI+) m/z 562.3 (M+H).

### Example 110-C

### (S)-2-(2-((3′-(1-Amino-2-hydroxyethyl)-5-ethyl-[1,1′-biphenyl]-3-yl)methoxy)phenyl)acetic acid

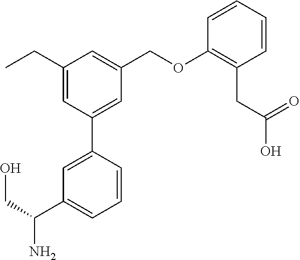 [SMILES: CCc1cc(COc2ccccc2CC(=O)O)cc(-c2cccc([C@H](N)CO)c2)c1]

The title compound was synthesized as described in Example 20-B starting with (S)-tert-butyl 2-(2-((3′-(1-((tert-butoxycarbonyl)amino)-2-hydroxyethyl)-5-ethyl-[1,1′-biphenyl]-3-yl)methoxy)phenyl)acetate. ¹H NMR (400 MHz, METHANOL-d₄) δ ppm 8.16 (s, 1H) 7.89 (s, 1H) 7.69 (d, J=8.08 Hz, 1H) 7.39-7.49 (m, 2H) 7.30 (d, J=7.71 Hz, 1H) 7.10-7.25 (m, 3H) 6.96 (d, J=7.83 Hz, 1H) 6.87 (td, J=7.39, 0.88 Hz, 1H) 5.08-5.25 (m, 2H) 4.34 (dd, J=8.84, 4.55 Hz, 1H) 3.95 (dd, J=11.68, 8.91 Hz, 1H) 3.85 (dd, J=11.68, 4.61 Hz, 1H) 3.49-3.69 (m, 2H) 2.73 (q, J=7.58 Hz, 2H) 1.29 (t, J=7.58 Hz, 3H). HRMS calcd. for C₂₅H₂₇NO₄ (M+H)⁺ 406.2018. found 406.2025.

### Example 111

### Example 111-A

### (S)-tert-Butyl 2-(2-((5-bromo-3′-(1-((tert-butoxycarbonyl)amino)-2-hydroxyethyl)-[1,1′-biphenyl]-3-yl)methoxy)phenyl)acetate

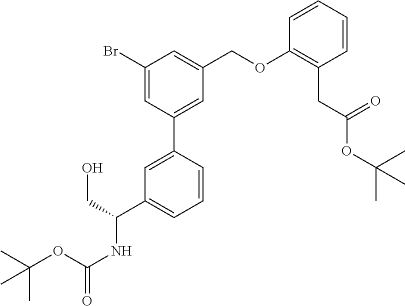 [SMILES: CC(C)(C)OC(=O)Cc1ccccc1OCc1cc(Br)cc(-c2cccc([C@@H](CO)NC(=O)OC(C)(C)C)c2)c1]

The title compound was synthesized as described in Example 102-A starting with tert-butyl 2-(2-((3,5-dibromobenzyl)oxy)phenyl)acetate (Intermediate 52) and (S)-tert-butyl (2-hydroxy-1-(3-(4,4,5,5-tetramethyl-1,3,2-dioxaborolan-2-yl)phenyl)ethyl)carbamate (Intermediate 34-B). MS (ESI+) m/z 612.2, 614.1 (M+H).

### Example 111-B

### (S)-2-(2-((3′-(1-amino-2-hydroxyethyl)-5-bromo-[1,1′-biphenyl]-3-yl)methoxy)phenyl)acetic acid

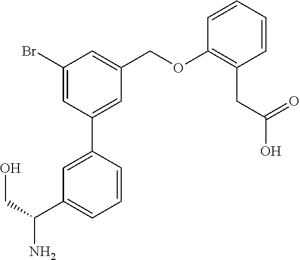 [SMILES: N[C@H](CO)c1cccc(-c2cc(Br)cc(COc3ccccc3CC(=O)O)c2)c1]

The title compound was synthesized as described in Example 20-B starting with (S)-tert-butyl 2-(2-((5-bromo-3′-(1-((tert-butoxycarbonyl)amino)-2-hydroxyethyl)-[1,1′-biphenyl]-3-yl)methoxy)phenyl)acetate. ¹H NMR (400 MHz, METHANOL-d₄) δ ppm 8.17 (s, 1H) 8.06 (s, 1H) 7.74-7.80 (m, 1H) 7.69 (d, J=7.96 Hz, 1H) 7.55 (s, 1H) 7.48 (t, J=7.71 Hz, 1H) 7.36 (d, J=7.71 Hz, 1H) 7.12-7.23 (m, 2H) 6.95 (d, J=7.71 Hz, 1H) 6.82-6.92 (m, 1H) 5.11-5.25 (m, 2H) 4.36 (dd, J=8.72, 4.55 Hz, 1H) 3.95 (dd, J=11.68, 8.78 Hz, 1H) 3.86 (dd, J=11.68, 4.61 Hz, 1H) 3.50-3.68 (m, 2H). HRMS calcd. for C₂₃H₂₂BrNO₄ (M+H)⁺ 456.0810 and 458.0790. found 456.0804 and 458.0809.

### Example 112

### Example 112-A

### (S)-tert-Butyl 2-(2-((3′-(1-((tert-butoxycarbonyl)amino)-2-hydroxyethyl)-5-methyl-[1,1′-biphenyl]-3-yl)methoxy)phenyl)acetate

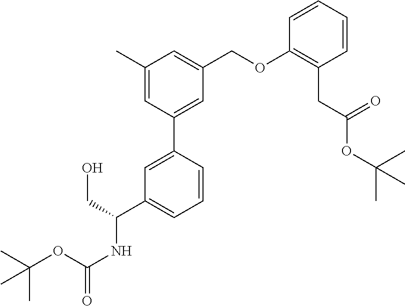 [SMILES: Cc1cc(COc2ccccc2CC(=O)OC(C)(C)C)cc(-c2cccc([C@@H](CO)NC(=O)OC(C)(C)C)c2)c1]

In a microwave vial, to a solution of (S)-tert-butyl 2-(2-((5-bromo-3′-(1-((tert-butoxycarbonyl)amino)-2-hydroxyethyl)-[1,1′-biphenyl]-3-yl)methoxy)phenyl)acetate (Example 111-A) (150 mg, 0.245 mmol) and potassium methyltrifluoroborate (CAS #13862-28-7) (90 mg, 0.73 mmol) in acetonitrile (2.18 mL) and water (0.272 mL) was added 2M aqueous K₃PO₄ (0.367 mL, 0.735 mmol) and PdCl₂(dppf).CH₂Cl₂ adduct (CAS #95464-05-4) (20.0 mg, 0.024 mmol) and the reaction was stirred at 110° C. for 1 hour. Additional 2M aq. K₃PO₄ (0.367 mL, 0.735 mmol) and PdCl₂(dppf).CH₂Cl₂ adduct (CAS #95464-05-4) (20.0 mg, 0.024 mmol) and potassium methyltrifluoroborate (90 mg, 0.73 mmol) were added and the reaction was heated at 110° C. for 2 hours. The reaction was diluted with water and EtOAc, extracted with EtOAc, dried over MgSO₄, filtered and concentrated. The crude product was purified by flash chromatography (0-100% EtOAc:Heptanes) to provide the title compound. MS (ESI+) m/z 548.2 (M+H).

### Example 112-B

### (S)-2-(2-((3′-(1-Amino-2-hydroxyethyl)-5-methyl-[1,1′-biphenyl]-3-yl)methoxy)phenyl)acetic acid

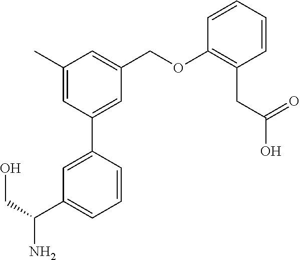 [SMILES: Cc1cc(COc2ccccc2CC(=O)O)cc(-c2cccc([C@H](N)CO)c2)c1]

The title compound was synthesized as described in Example 20-B starting with (S)-tert-butyl 2-(2-((3′-(1-((tert-butoxycarbonyl)amino)-2-hydroxyethyl)-5-methyl-[1,1′-biphenyl]-3-yl)methoxy)phenyl)acetate. ¹H NMR (400 MHz, METHANOL-d₄) δ ppm 8.15 (s, 1H) 7.88 (s, 1H) 7.69 (d, J=7.83 Hz, 1H) 7.39-7.51 (m, 2H) 7.30 (d, J=7.71 Hz, 1H) 7.09-7.24 (m, 3H) 6.95 (d, J=8.08 Hz, 1H) 6.80-6.92 (m, 1H) 5.09-5.22 (m, 2H) 4.34 (dd, J=8.97, 4.55 Hz, 1H) 3.95 (dd, J=11.62, 8.97 Hz, 1H) 3.85 (dd, J=11.68, 4.61 Hz, 1H) 3.50-3.68 (m, 2H) 2.42 (s, 3H). HRMS calcd. for C₂₄H₂₅NO₄ (M+H)⁺ 392.1862. found 392.1850.

### Example 113

### Example 113-A

### tert-Butyl 2-(2-((3′-(((tert-butoxycarbonyl)amino)methyl)-5-cyano-[1,1′-biphenyl]-3-yl)methoxy)phenyl)acetate

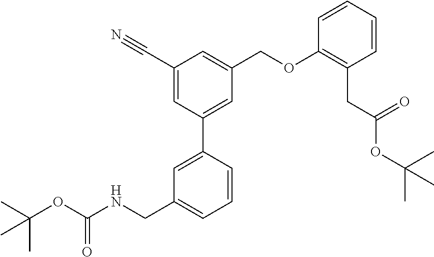 [SMILES: CC(C)(C)OC(=O)Cc1ccccc1OCc1cc(C#N)cc(-c2cccc(CNC(=O)OC(C)(C)C)c2)c1]

The title compound was synthesized as described in Example 102-A starting with tert-butyl 2-(2-((3-bromo-5-cyanobenzyl)oxy)phenyl)acetate (Intermediate 54). MS (ESI−) m/z 527.2 (M−H).

### Example 113-B

### 2-(2-((3′-(Aminomethyl)-5-cyano-[1,1′-biphenyl]-3-yl)methoxy)phenyl)acetic acid

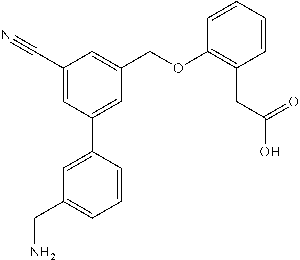 [SMILES: N#Cc1cc(COc2ccccc2CC(=O)O)cc(-c2cccc(CN)c2)c1]

The title compound was synthesized as described in Example 20-B starting with tert-butyl 2-(2-((3′-(((tert-butoxycarbonyl)amino)methyl)-5-cyano-[1,1′-biphenyl]-3-yl)methoxy)phenyl)acetate. ¹H NMR (400 MHz, METHANOL-d₄) δ ppm 8.37 (s, 1H) 8.20 (s, 1H) 7.99 (s, 1H) 7.68-7.79 (m, 2H) 7.49 (t, J=7.71 Hz, 1H) 7.39 (d, J=7.83 Hz, 1H) 7.12-7.25 (m, 2H) 6.95 (d, J=7.96 Hz, 1H) 6.85-6.93 (m, 1H) 5.25 (s, 2H) 4.18 (s, 2H) 3.60 (s, 2H). HRMS calcd. for C₂₃H₂₀N₂O₃ (M+H)⁺ 373.1552. found 373.1553.

### Example 114

### Example 114-A

### (S)-tert-Butyl 2-(2-((3′-(1-((tert-butoxycarbonyl)amino)-2-hydroxyethyl)-5-(trifluoromethyl)-[1,1′-biphenyl]-3-yl)methoxy)phenyl)acetate

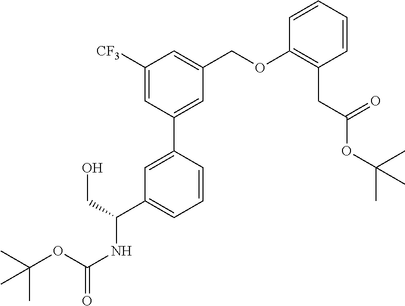 [SMILES: CC(C)(C)OC(=O)Cc1ccccc1OCc1cc(-c2cccc([C@@H](CO)NC(=O)OC(C)(C)C)c2)cc(C(F)(F)F)c1]

The title compound was synthesized as described in Example 102-A starting with tert-butyl 2-(2-((3-bromo-5-(trifluoromethyl)benzyl)oxy)phenyl)acetate (Intermediate 55) and (S)-tert-butyl (2-hydroxy-1-(3-(4,4,5,5-tetramethyl-1,3,2-dioxaborolan-2-yl)phenyl)ethyl)carbamate (Intermediate 34-B). MS (ESI+) m/z 602.3 (M+H).

### Example 114-B

### (S)-2-(2-((3′-(1-Amino-2-hydroxyethyl)-5-(trifluoromethyl)-[1,1′-biphenyl]-3-yl)methoxy)phenyl)acetic acid

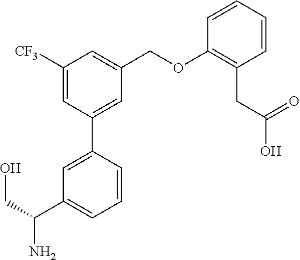 [SMILES: N[C@H](CO)c1cccc(-c2cc(COc3ccccc3CC(=O)O)cc(C(F)(F)F)c2)c1]

(S)-tert-butyl 2-(2-((3′-(1-((tert-butoxycarbonyl)amino)-2-hydroxyethyl)-5-(trifluoromethyl)-[1,1′-biphenyl]-3-yl)methoxy)phenyl)acetate (0.145 g, 0.241 mmol) was dissolved in dioxane (2.41 mL) and HCl (4M in Dioxane, 3.62 mL, 14.5 mmol) was added. After stirring overnight the reaction was concentrated and purified by prep. HPLC (Method B) to provide the title compound. ¹H NMR (400 MHz, METHANOL-d₄) δ ppm 8.35 (s, 1H) 8.24 (s, 1H) 7.88 (s, 1H) 7.75 (d, J=8.34 Hz, 1H) 7.69 (s, 1H) 7.51 (t, J=7.71 Hz, 1H) 7.39 (d, J=7.71 Hz, 1H) 7.14-7.24 (m, 2H) 6.98 (d, J=7.83 Hz, 1H) 6.82-6.94 (m, 1H) 5.19-5.34 (m, 2H) 4.37 (dd, J=8.78, 4.61 Hz, 1H) 3.97 (dd, J=11.68, 8.78 Hz, 1H) 3.87 (dd, J=11.68, 4.61 Hz, 1H) 3.51-3.69 (m, 2H). HRMS calcd. for C₂₄H₂₂F₃NO₄ (M+H)⁺ 446.1579. found 446.1569.

### Example 115

The following compounds were prepared with similar methods as described in Examples 102-A and 114-B using the aryl bromides from Intermediates 56-58 and (S)-tert-butyl (2-hydroxy-1-(3-(4,4,5,5-tetramethyl-1,3,2-dioxaborolan-2-yl)phenyl)ethyl)carbamate (Intermediate 34-B).

| | Structure/Chemical Name | ¹H NMR | HRMS |
|---|---|---|---|
| 115-A | 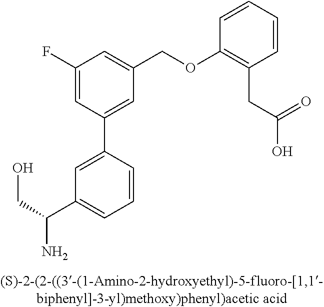 [SMILES: N[C@H](CO)c1cccc(-c2cc(F)cc(COc3ccccc3CC(=O)O)c2)c1] | (400 MHz, METHANOL-d₄) δ ppm 8.20 (s, 1 H) 7.93 (s, 1 H) 7.65-7.73 (m, 1 H) 7.47 (t, J = 7.71 Hz, 1 H) 7.30-7.40 (m, 2 H) 7.09-7.23 (m, 3 H) 6.95 (d, J = 7.83 Hz, 1 H) 6.88 (td, J = 7.39, 1.01 Hz, 1 H) 5.12-5.25 (m, 2 H) 4.35 (dd, J = 8.78, 4.61 Hz, 1 H) 3.95 (dd, J = 11.75, 8.84 Hz, 1 H) 3.86 (dd, J = 11.68, 4.61 Hz, 1 H) 3.51-3.68 (m, 2 H) | calcd. for C₂₃H₂₂FNO₄ (M + H)⁺ 396.1611, found 396.1597. |
| 115-B | 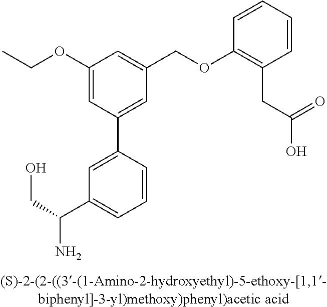 [SMILES: CCOc1cc(COc2ccccc2CC(=O)O)cc(-c2cccc([C@H](N)CO)c2)c1] | (400 MHz, METHANOL-d₄) δ ppm 8.15 (s, 1 H) 7.63-7.71 (m, 2 H) 7.45 (t, J = 7.71 Hz, 1 H) 7.31 (d, J = 7.71 Hz, 1 H) 7.08-7.22 (m, 3 H) 6.90-6.99 (m, 2 H) 6.87 (td, J = 7.39, 1.01 Hz, 1 H) 5.08-5.22 (m, 2 H) 4.34 (dd, J = 8.84, 4.55 Hz, 1 H) 4.12 (q, J = 7.03 Hz, 2 H) 3.95 (dd, J = 11.68, 8.91 Hz, 1 H) 3.85 (dd, J = 11.68, 4.61 Hz, 1 H) 3.50-3.68 (m, 2 H) 1.42 (t, J = 6.95 Hz, 3 H) | calcd. for C₂₅H₂₇NO₅ (M + H)⁺ 422.1967, found 422.1960. |
| 115-C | 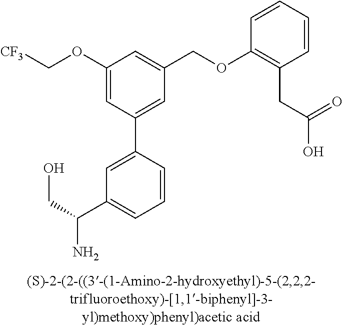 [SMILES: N[C@H](CO)c1cccc(-c2cc(COc3ccccc3CC(=O)O)cc(OCC(F)(F)F)c2)c1] | (400 MHz, METHANOL-d₄) δ ppm 8.17 (s, 1 H) 7.79 (s, 1 H) 7.72 (d, J = 7.83 Hz, 1 H) 7.47 (t, J = 7.71 Hz, 1 H) 7.34 (d, J = 7.71 Hz, 1 H) 7.24 (s, 1 H) 7.12-7.22 (m, 2 H) 7.05 (s, 1 H) 6.95 (d, J = 7.83 Hz, 1 H) 6.87 (t, J = 7.01 Hz, 1 H) 5.10-5.25 (m, 2 H) 4.63 (q, J = 8.55 Hz, 2 H) 4.35 (dd, J = 8.78, 4.61 Hz, 1 H) 3.95 (dd, J = 11.75, 8.84 Hz, 1 H) 3.86 (dd, J = 11.68, 4.61 Hz, 1 H) 3.51-3.68 (m, 2 H) | calcd. for C₂₅H₂₄F₃NO₅ (M + H)⁺ 476.1685, found 476.1667. |

### Example 116

The following compounds were prepared with a similar method as described in Example 108-A, using the aryl bromides from Intermediates 59-67 and the appropriate boronic acid or boronic ester [(3-(((tert-butoxycarbonyl)amino)methyl)phenyl)boronic acid (CAS #199609-62-6) or Intermediate 26 or Intermediate 34-B, and then deprotected using the methods of Examples 20-B, 114-B, or 108-B.

| | | Method of | | |
|---|---|---|---|---|
| | Structure/Chemical Name | deprotection | ¹H NMR | HRMS |
| 116-A | 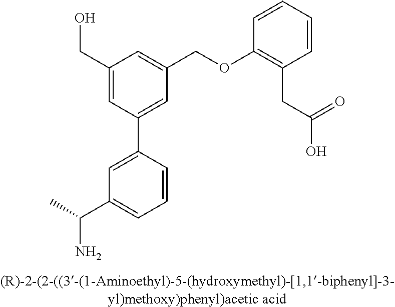 [SMILES: C[C@@H](N)c1cccc(-c2cc(CO)cc(COc3ccccc3CC(=O)O)c2)c1] | 20-B | (400 MHz, METHANOL-d₄) δ ppm 8.16-8.23 (m, 1 H) 8.01 (s, 1 H) 7.66-7.73 (m, 1 H) 7.61 (s, 1 H) 7.45 (t, J = 7.71 Hz, 1 H) 7.27-7.38 (m, 2 H) 7.11-7.21 (m, 2 H) 6.96 (d, J = 7.83 Hz, 1 H) 6.87 (td, J = 7.39, 1.01 Hz, 1 H) 5.12-5.26 (m, 2 H) 4.70 (s, 2 H) 4.47 (q, J = 6.82 Hz, 1 H) 3.52-3.69 (m, 2 H) 1.67 (d, J = 6.95 Hz, 3 H). | calcd. for C₂₄H₂₅NO₄ (M + H)⁺ 392.1862, found 392.1862. |
| 116-B | 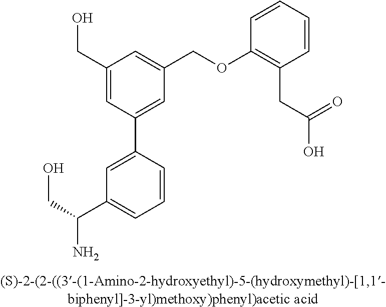 [SMILES: N[C@H](CO)c1cccc(-c2cc(CO)cc(COc3ccccc3CC(=O)O)c2)c1] | 20-B | (400 MHz, METHANOL-d₄) δ ppm 8.17 (s, 1 H) 7.99 (s, 1 H) 7.73 (d, J = 8.07 Hz, 1 H) 7.61 (s, 1 H) 7.46 (t, J = 7.70 Hz, 1 H) 7.29-7.38 (m, 2 H) 7.12-7.21 (m, 2 H) 6.96 (d, J = 7.70 Hz, 1 H) 6.87 (td, J = 7.37, 0.92 Hz, 1 H) 5.14-5.26 (m, 2 H) 4.70 (s, 2 H) 4.35 (dd, J = 8.86, 4.58 Hz, 1 H) 3.95 (dd, J = 11.74, 8.93 Hz, 1 H) 3.86 (dd, J = 11.68, 4.58 Hz, 1 H) 3.51-3.68 (m, 2 H). | calcd. for C₂₄H₂₅NO₅ (M + H)⁺ 408.1811, found 408.1802. |
| 116-C | 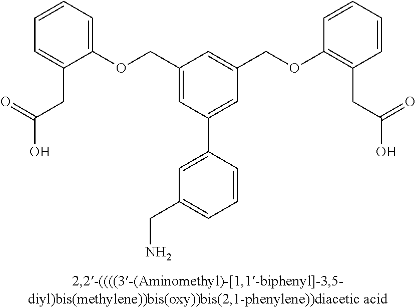 [SMILES: NCc1cccc(-c2cc(COc3ccccc3CC(=O)O)cc(COc3ccccc3CC(=O)O)c2)c1] | 20-B | (400 MHz, METHANOL-d₄) δ ppm 8.15 (s, 1 H) J = 6.82 Hz, 1 H) 7.41-7.49 (m, 2H) 7.32 (d, J = 8.34 Hz, 1 H) 7.14-7.24 (m, 4H) 7.00 (d, J = 8.72 Hz, 2 H) 6.85-6.94 (m, 2H) 5.22 (s, 4 H) 4.18 (s, 2 H) 3.65 (s, 4 H). | calcd. for C₃₁H₂₉NO₆ (M + H)⁺ 512.2073, found 512.2056. |
| 116-D | 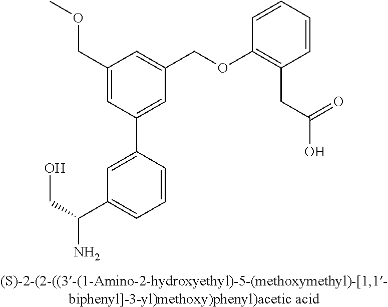 [SMILES: COCc1cc(COc2ccccc2CC(=O)O)cc(-c2cccc([C@H](N)CO)c2)c1] | 20-B | (400 MHz, METHANOL-d₄) δ ppm 7.91 (s, 1 H) 7.82 (s, 1 H) 7.73 (dt, J = 8.08, 1.26 Hz, 1 H) 7.59 (s, 1 H) 7.52 (t, J = 7.77 Hz, 1 H) 7.36-7.45 (m, 2 H) 7.16-7.25 (m, 2 H) 6.97 - 7.06 (m, 1 H) 6.90 (td, J = 7.45, 1.01 Hz, 1 H) 5.20 (s, 2 H) 4.55 (s, 2H) 4.41 (dd, J = 7.83, 4.93 Hz, 1 H) 3.85-3.98 (m, 2 H) 3.59-3.73 (m, 2 H) 3.42 (s, 3 H). | calcd. for C₂₅H₂₇NO₅ (M + H)⁺ 422.1967, found 422.1954. |
| 116-E a) | 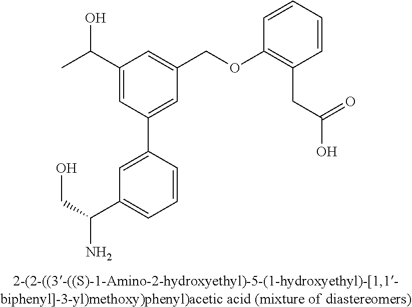 [SMILES: CC(O)c1cc(COc2ccccc2CC(=O)O)cc(-c2cccc([C@H](N)CO)c2)c1] | 114-B | (400 MHz, METHANOL-d₄) δ ppm 8.16 (s, 1 H) 7.97 (s, 1 H) 7.72 (d, J = 7.71 Hz, 1 H) 7.62 (s, 1 H) 7.46 (t, J = 7.71 Hz, 1 H) 7.36 (s, 1 H) 7.32 (d, J = 7.71 Hz, 1 H) 7.11-7.23 (m, 2 H) 6.96 (d, J = 7.71 Hz, 1 H) 6.83-6.92 (m, 1 H) 5.13-5.25 (m, 2 H) 4.89-4.96 (m, 1 H) 4.34 (dd, J = 8.84, 4.55 Hz, 1 H) 3.94 (dd, J = 11.68, 8.91 Hz, 1 H) 3.85 (dd, J = 11.62, 4.55 Hz, 1 H) 3.51-3.68 (m, 2 H) 1.50 (d, J = 6.44 Hz, 3 H). | calcd. for C₂₅H₂₇NO₅ (M + H)⁺ 422.1967, found 422.1964. |
| 116-E | Resolution of the diastereomers of 2-(2-((3′-((S)-1-amino-2-hydroxyethyl)-5-(1-hydroxyethyl)-[1,1′-biphenyl]-3-yl)methoxy)phenyl)acetic | | | |
| b) | acid was achieved by chiral SFC using a CHIRALPAK ® AD-H column with 25% MeOH and 5 mM NH₄OH in CO₂ to give the first | | | |
| | diastereomer (tᵣ = 3.8 min) and the second diastereomer (tᵣ = 5.7 min). | | | |
| | First diastereomer (tᵣ = 3.8 min): ¹H NMR (400 MHz, METHANOL-d₄) δ ppm 8.17 (s, 1 H) 7.98 (s, 1 H) 7.72 (d, J = 8.08 Hz, 1 H) 7.62 (s, 1 | | | |
| | H) 7.46 (t, J = 7.71 Hz, 1 H) 7.36 (s, 1 H) 7.32 (d, J = 7.58 Hz, 1 H) 7.12-7.22 (m, 2 H) 6.96 (d, J = 7.71 Hz, 1 H) 6.87 (td, J = 7.39, 1.01 Hz, | | | |
| | 1 H) 5.13-5.26 (m, 2 H) 4.92 (q, J = 6.44 Hz, 1 H) 4.35 (dd, J = 8.91, 4.48 Hz, 1 H) 3.95 (dd, J = 11.68, 8.91 Hz, 1 H) 3.86 (dd, J = 11.75, | | | |
| | 4.55 Hz, 1 H) 3.52-3.68 (m, 2 H) 1.50 (d, J = 6.44 Hz, 3 H). HRMS calcd. for C₂₅H₂₇NO₅ (M + H)⁺ 422.1967, found 422.1961. | | | |
| | Second diastereomer (tᵣ = 5.7 min): ¹H NMR (400 MHz, METHANOL-d₄) δ ppm 8.18 (s, 1 H) 7.98 (s, 1 H) 7.72 (d, J = 7.83 Hz, 1 H) 7.62 (s, | | | |
| | 1 H) 7.46 (t, J = 7.71 Hz, 1 H) 7.36 (s, 1 H) 7.32 (d, J = 7.58 Hz, 1 H) 7.09-7.22 (m, 2 H) 6.96 (d, J = 7.83 Hz, 1 H) 6.87 (td, J = 7.39, 0.88 | | | |
| | Hz, 1 H) 5.11-5.25 (m, 2 H) 4.88-4.96 (m, 1 H) 4.35 (dd, J = 8.78, 4.48 Hz, 1 H) 3.96 (dd, J = 11.68, 8.91 Hz, 1 H) 3.86 (dd, J = 11.68, 4.61 | | | |
| | Hz, 1 H) 3.50-3.69 (m, 2 H) 1.50 (d, J = 6.44 Hz, 3 H). HRMS calcd. for C₂₅H₂₇NO₅ (M + H)⁺ 422.1967, found 422.1958. | | | |
| 116-F | 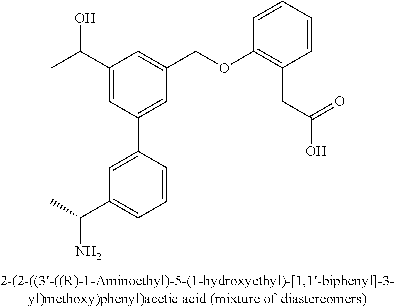 [SMILES: CC(O)c1cc(COc2ccccc2CC(=O)O)cc(-c2cccc([C@@H](C)N)c2)c1] | 114-B | (400 MHz, METHANOL-d₄) δ ppm 8.13 (s, 1 H) 7.94 (s, 1 H) 7.66-7.74 (m, 1 H) 7.62 (s, 1 H) 7.46 (t, J = 7.71 Hz, 1 H) 7.30-7.41 (m, 2 H) 7.13-7.24 (m, 2 H) 6.97 (d, J = 7.83 Hz, 1 H) 6.88 (td, J = 7.39, 1.01 Hz, 1 H) 5.11-5.25 (m, 2 H) 4.92 (q, J = 6.44 Hz, 1 H) 4.47 (q, J = 6.82 Hz, 1 H) 3.50-3.71 (m, 2 H) 1.67 (d, J = 6.95 Hz, 3 H) 1.50 (d, J = 6.44 Hz, 3 H). | calcd. for C₂₅H₂₇NO₄ (M + H)⁺ 406.2018, found 406.2011. |
| 116-G a) | 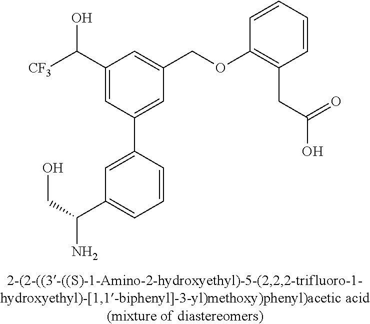 [SMILES: N[C@H](CO)c1cccc(-c2cc(COc3ccccc3CC(=O)O)cc(C(O)C(F)(F)F)c2)c1] | 20-B | (400 MHz, METHANOL-d₄) δ ppm 8.21 (s, 1 H) 8.11 (s, 1 H) 7.67-7.77 (m, 2 H) 7.43-7.52 (m, 2 H) 7.34 (d, J = 7.58 Hz, 1 H) 7.13-7.21 (m, 2 H) 6.97 (d, J = 7.96 Hz, 1 H) 6.88 (td, J = 7.39, 1.01 Hz, 1 H) 5.17-5.27 (m, 2 H) 5.08-5.17 (m, 1 H) 4.36 (dd, J = 8.78, 4.61 Hz, 1 H) 3.96 (dd, J = 11.75, 8.84 Hz, 1 H) 3.87 (dd, J = 11.68, 4.61 Hz, 1 H) 3.51-3.68 (m, 2 H). | calcd. for C₂₅H₂₄F₃NO₅ (M + H)⁺ 476.1685, found 476.1675. |
| 116-G | Resolution of the diastereomers of 2-(2-((3′-((S)-1-amino-2-hydroxyethyl)-5-(2,2,2-trifluoro-1-hydroxyethyl)-[1,1′-biphenyl]-3- | | | |
| b) | yl)methoxy)phenyl)acetic acid was achieved by chiral SFC using a CHIRALPAK ® AD-H column with 25% MeOH and 5 mM NH₄OH in | | | |
| | CO₂ to give the first diastereomer (tᵣ = 2.1 min) and the second diastereomer (tᵣ = 3.4 min). | | | |
| | First diastereomer (tᵣ = 2.1 min): ¹H NMR (400 MHz, METHANOL-d₄) δ ppm 8.21 (s, 1 H) 8.11 (s, 1 H) 7.66-7.78 (m, 2 H) 7.41-7.52 (m, 2 | | | |
| | H) 7.34 (d, J = 7.70 Hz, 1 H) 7.11-7.22 (m, 2 H) 6.97 (d, J = 8.08 Hz, 1 H) 6.81-6.93 (m, 1 H) 5.17-5.27 (m, 2 H) 5.07-5.17 (m, 1 H) 4.36 (dd, | | | |
| | J = 8.84, 4.55 Hz, 1 H) 3.96 (dd, J = 11.75, 8.84 Hz, 1 H) 3.87 (dd, J = 11.62, 4.55 Hz, 1 H) 3.51-3.69 (m, 2 H). HRMS calcd. for | | | |
| | C₂₅H₂₄F₃NO₅ (M + H)⁺ 476.1685, found 476.1673. | | | |
| | Second diastereomer (tᵣ = 3.4 min): ¹H NMR (400 MHz, METHANOL-d₄) δ ppm 8.21 (s, 1 H) 8.11 (s, 1 H) 7.67-7.76 (m, 2 H) 7.45-7.52 (m, | | | |
| | 2 H) 7.34 (d, J = 7.71 Hz, 1 H) 7.13-7.22 (m, 2 H) 6.97 (d, J = 7.96 Hz, 1 H) 6.82-6.93 (m, 1 H) 5.18-5.28 (m, 2 H) 5.09-5.18 (m, 1 H) 4.36 | | | |
| | (dd, J = 8.91, 4.48 Hz, 1 H) 3.96 (dd, J = 11.62, 8.84 Hz, 1 H) 3.87 (dd, J = 11.68, 4.61 Hz, 1 H) 3.50-3.69 (m, 2 H). HRMS calcd. for | | | |
| | C₂₅H₂₄F₃NO₅ (M + H)⁺ 476.1685, found 476.1670. | | | |
| 116-H a) | 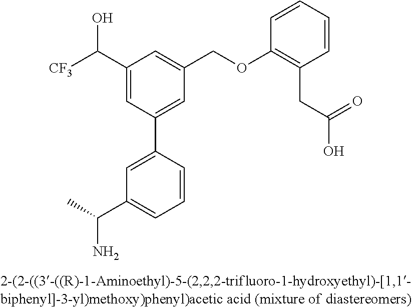 [SMILES: C[C@@H](N)c1cccc(-c2cc(COc3ccccc3CC(=O)O)cc(C(O)C(F)(F)F)c2)c1] | 20-B | (400 MHz, METHANOL-d₄) δ ppm 8.23 (s, 1 H) 8.12 (s, 1 H) 7.73 (s, 1 H) 7.68 (d, J = 7.83 Hz, 1 H) 7.41-7.52 (m, 2 H) 7.34 (d, J = 7.70 Hz, 1 H) 7.11-7.22 (m, 2 H) 6.98 (d, J = 7.83 Hz, 1 H) 6.88 (td, J = 7.39, 0.88 Hz, 1 H) 5.06-5.30 (m, 3 H) 4.48 (q, J = 6.91 Hz, 1 H) 3.50-3.70 (m, 2 H) 1.68 (d, J = 6.82 Hz, 3 H). | calcd. for C₂₅H₂₄F₃NO₄ (M + H)⁺ 460.1736, found 460.1725. |
| 116-H | Resolution of the diastereomers of 2-(2-((3′-((R)-1-aminoethyl)-5-(2,2,2-trifluoro-1-hydroxyethyl)-[1,1′-biphenyl]-3- | | | |
| b) | yl)methoxy)phenyl)acetic acid was achieved by chiral SFC using a CHIRALPAK ® AD-H column with 20% MeOH and 5 mM NH₄OH in | | | |
| | CO₂ to give the first diastereomer (tᵣ = 3.0 min) and the second diastereomer (tᵣ = 4.6 min). | | | |
| | First diastereomer (tᵣ = 3.0 min): ¹H NMR (400 MHz, METHANOL-d₄) δ ppm 8.23 (s, 1 H) 8.12 (s, 1 H) 7.73 (s, 1 H) 7.68 (d, J = 7.83 Hz, 1 | | | |
| | H) 7.42-7.52 (m, 2 H) 7.34 (d, J = 7.45 Hz, 1 H) 7.13-7.22 (m, 2 H) 6.98 (d, J = 7.96 Hz, 1 H) 6.88 (t, J = 6.95 Hz, 1 H) 5.09-5.28 (m, 3 H) | | | |
| | 4.48 (q, J = 6.91 Hz, 1 H) 3.50-3.70 (m, 2 H) 1.68 (d, J = 6.95 Hz, 3 H). HRMS calcd. for C₂₅H₂₄F₃NO₄ (M + H)⁺ 460.1736, found 460.1720. | | | |
| | Second diastereomer (tᵣ = 4.6 min): ¹H NMR (400 MHz, METHANOL-d₄) δ ppm 8.23 (s, 1 H) 8.12 (s, 1 H) 7.73 (s, 1 H) 7.68 (d, J = 7.83 Hz, | | | |
| | 1 H) 7.42-7.53 (m, 2 H) 7.34 (d, J = 7.58 Hz, 1 H) 7.12-7.23 (m, 2 H) 6.98 (d, J = 7.83 Hz, 1 H) 6.88 (td, J = 7.39, 1.01 Hz, 1 H) 5.09-5.29 (m, | | | |
| | 3 H) 4.48 (q, J = 6.86 Hz, 1 H) 3.51-3.70 (m, 2 H) 1.68 (d, J = 6.95 Hz, 3 H). HRMS calcd. for C₂₅H₂₄F₃NO₄ (M + H)⁺ 460.1736, found | | | |
| | 460.1721. | | | |
| 116-I a) | 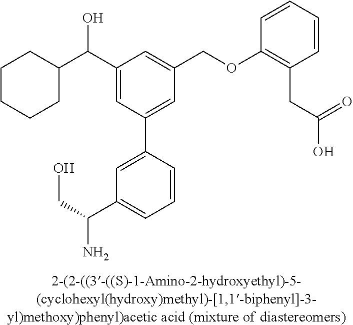 [SMILES: N[C@H](CO)c1cccc(-c2cc(COc3ccccc3CC(=O)O)cc(C(O)C3CCCCC3)c2)c1] | 114-B | (400 MHz, METHANOL-d₄) δ ppm 8.19 (s, 1 H) 7.97 (s, 1 H) 7.70 (d, J = 8.08 Hz, 1 H) 7.52 (br. s., 1 H) 7.46 (t, J = 7.71 Hz, 1 H) 7.25-7.35 (m, 2 H) 7.10-7.23 (m, 2 H) 6.97 (d, J = 7.71 Hz, 1 H) 6.87 (t, J = 6.95 Hz, 1 H) 5.12-5.26 (m, 2 H) 4.40 (d, J = 5.43 Hz, 1 H) 4.35 (dd, J = 8.65, 4.36 Hz, 1 H) 3.96 (dd, J = 11.68, 8.91 Hz, 1 H) 3.86 (dd, J = 11.62, 4.55 Hz, 1 H) 3.51-3.68 (m, 2 H) 2.02 (d, J = 11.75 Hz, 1 H) 1.78 (d, J = 12.76 Hz, 1 H) 1.56-1.72 (m, 3 H) 1.41 (d, J = 12.00 Hz, 1 H) 0.92-1.34 (m, 5 H). | calcd. for C₃₀H₃₅NO₅ (M + H)⁺ 490.2593, found 490.2597. |
| 116-I | Resolution of the diastereomers of 2-(2-((3′-((S)-1-amino-2-hydroxyethyl)-5-(cyclohexyl(hydroxy)methyl)-[1,1′-biphenyl]-3- | | | |
| b) | yl)methoxy)phenyl)acetic acid was achieved by chiral SFC using a CHIRALPAK ® AD-H column with 30% MeOH and 10 mM NH₄OH in | | | |
| | CO₂ to give the first diastereomer (tᵣ = 4.0 min) and the second diastereomer (tᵣ = 6.3 min). | | | |
| | First diastereomer (tᵣ = 4.0 min): ¹H NMR (400 MHz, METHANOL-d₄) δ ppm 8.18 (s, 1 H) 7.97 (s, 1 H) 7.70 (d, J = 7.96 Hz, 1 H) 7.52 (s, 1 | | | |
| | H) 7.46 (t, J = 7.71 Hz, 1 H) 7.25-7.36 (m, 2 H) 7.10-7.21 (m, 2 H) 6.96 (d, J = 7.83 Hz, 1 H) 6.80-6.92 (m, 1 H) 5.12-5.28 (m, 2 H) 4.40 (d, | | | |
| | J = 7.07 Hz, 1 H) 4.35 (dd, J = 8.91, 4.61 Hz, 1 H) 3.96 (dd, J = 11.68, 8.91 Hz, 1 H) 3.86 (dd, J = 11.68, 4.61 Hz, 1 H) 3.50-3.68 (m, 2 H) | | | |
| | 2.02 (d, J = 12.13 Hz, 1 H) 1.78 (d, J = 12.88 Hz, 1 H) 1.54-1.73 (m, 3 H) 1.41 (d, J = 12.51 Hz, 1 H) 0.92-1.30 (m, 5 H). HRMS calcd. for | | | |
| | C₃₀H₃₅NO₅ (M + H)⁺ 490.2593, found 490.2606. | | | |
| | Second diastereomer (tᵣ = 6.3 min): ¹H NMR (400 MHz, METHANOL-d₄) δ ppm 8.18 (s, 1 H) 7.97 (s, 1 H) 7.70 (d, J = 8.08 Hz, 1 H) 7.53 (s, | | | |
| | 1 H) 7.46 (t, J = 7.71 Hz, 1 H) 7.24-7.35 (m, 2 H) 7.16 (qd, J = 7.37, 1.64 Hz, 2 H) 6.96 (d, J = 7.71 Hz, 1 H) 6.87 (td, J = 7.36, 0.95 Hz, 1 H) | | | |
| | 5.10-5.28 (m, 2 H) 4.40 (d, J = 7.20 Hz, 1 H) 4.35 (dd, J = 8.84, 4.55 Hz, 1 H) 3.96 (dd, J = 11.68, 8.91 Hz, 1 H) 3.86 (dd, J = 11.75, 4.67 Hz, | | | |
| | 1 H) 3.50-3.70 (m, 2 H) 2.02 (d, J = 13.01 Hz, 1 H) 1.78 (d, J = 12.76 Hz, 1 H) 1.54-1.73 (m, 3 H) 1.41 (d, J = 12.25 Hz, 1 H) 0.94-1.30 (m, 5 | | | |
| | H). HRMS calcd. for C₃₀H₃₅NO₅ (M + H)⁺ 490.2593, found 490.2614. | | | |
| 116-J | 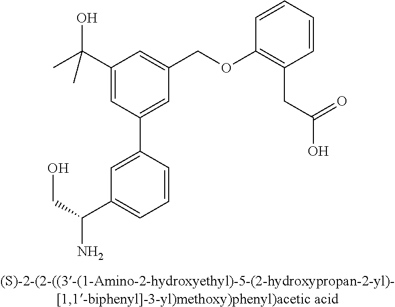 [SMILES: CC(C)(O)c1cc(COc2ccccc2CC(=O)O)cc(-c2cccc([C@H](N)CO)c2)c1] | 114-B | (400 MHz, METHANOL-d₄) δ ppm 8.17 (s, 1 H) 7.95 (s, 1 H) 7.67-7.78 (m, 2 H) 7.42-7.52 (m, 2 H) 7.32 (d, J = 7.71 Hz, 1 H) 7.11-7.22 (m, 2 H) 6.97 (d, J = 7.83 Hz, 1 H) 6.83-6.91 (m, 1 H) 5.13-5.26 (m, 2 H) 4.35 (dd, J = 8.97, 4.55 Hz, 1 H) 3.96 (dd, J = 11.62, 8.97 Hz, 1 H) 3.86 (dd, J = 11.68, 4.61 Hz, 1 H) 3.50-3.69 (m, 2 H) 1.60 (s, 6 H). | calcd. for C₂₆H₂₉NO₅ (M + H)⁺ 436.2124, found 436.2121. |
| 116-K | 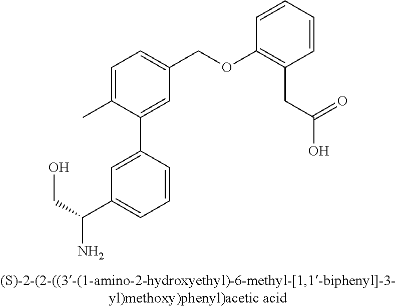 [SMILES: Cc1ccc(COc2ccccc2CC(=O)O)cc1-c1cccc([C@H](N)CO)c1] | 108-B | (400 MHz, METHANOL-d₄) δ ppm 7.76 (s, 1 H) 7.60 (s, 1 H) 7.38-7.53 (m, 2 H) 7.31-7.38 (m, 1 H) 7.23-7.29 (m, 2 H) 7.08-7.20 (m, 2 H) 6.97 (d, J = 7.83 Hz, 1 H) 6.78-6.90 (m, 1 H) 4.99-5.21 (m, 2 H) 4.34 (dd, J = 8.53, 4.74 Hz, 1 H) 3.81-4.02 (m, 2 H) 3.41-3.62 (m, 2 H) 2.34 (s, 3 H). | calcd. for C₂₄H₂₆NO₄ (M + H)⁺ 392.1862, found 392.1885. |
| 116-L | 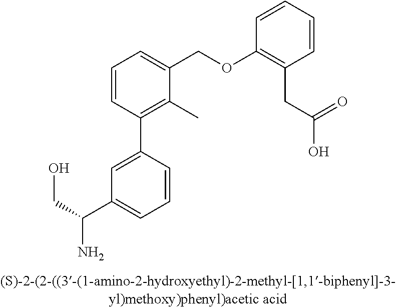 [SMILES: Cc1c(COc2ccccc2CC(=O)O)cccc1-c1cccc([C@H](N)CO)c1] | 108-B | (400 MHz, METHANOL-d₄) δ ppm 7.52 (s, 1 H) 7.48 (d, J = 7.71 Hz, 1 H) 7.43 (d, J = 7.33 Hz, 1 H) 7.35-7.40 (m, 2 H) 7.15-7.24 (m, 4 H) 7.03 (d, J = 8.21 Hz, 1 H) 6.87 (t, J = 7.45 Hz, 1 H) 5.03-5.22 (m, 2 H) 4.32 (dd, J = 8.53, 4.61 Hz, 1 H) 3.76-3.91 (m, 2 H) 3.41-3.59 (m, 2 H) 2.26 (s, 3 H). | calcd. for C₂₄H₂₆NO₄ (M + H)⁺ 392.1862, found 392.1880. |
| 116-M | 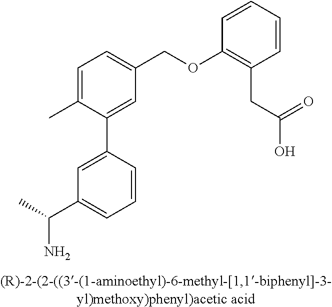 [SMILES: Cc1ccc(COc2ccccc2CC(=O)O)cc1-c1cccc([C@@H](C)N)c1] | 108-B | (400 MHz, METHANOL-d₄) δ ppm 7.79 (s, 1 H) 7.60 (s, 1 H) 7.38-7.48 (m, 2 H) 7.31-7.37 (m, 1 H) 7.25 (d, J = 0.88 Hz, 2 H) 7.12-7.19 (m, 2 H) 6.97 (d, J = 7.83 Hz, 1 H) 6.86 (td, J = 7.36, 0.95 Hz, 1 H) 5.09 (q, J = 11.62 Hz, 2 H) 4.46 (d, J = 6.95 Hz, 1 H) 3.41-3.63 (m, 2 H) 2.34 (s, 3 H) 1.68 (d, J = 6.95 Hz, 3 H). | calcd. for C₂₄H₂₅NO₃ (M + H)⁺ 376.1913, found 376.1919. |
| 116-N | 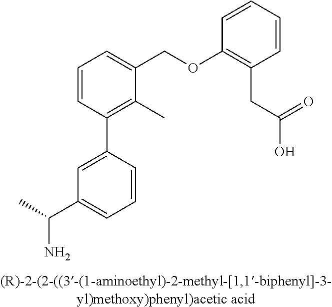 [SMILES: Cc1c(COc2ccccc2CC(=O)O)cccc1-c1cccc([C@@H](C)N)c1] | 108-B | (400 MHz, METHANOL-d₄) δ ppm 7.55 (s, 1 H) 7.47-7.52 (m, 1 H) 7.43 (dd, J = 7.07, 1.52 Hz, 1 H) 7.37 (dd, J = 7.45, 1.52 Hz, 2 H) 7.15-7.24 (m, 4 H) 7.04 (d, J = 7.71 Hz, 1 H) 6.83-6.90 (m, 1 H) 5.04-5.22 (m, 2 H) 4.40-4.51 (m, 1 H) 3.38-3.61 (m, 2 H) 2.26 (s, 3 H) 1.63 (d, J = 6.82 Hz, 3 H). | calcd. for C₂₄H₂₅NO₃ (M + H)⁺ 376.1913, found 376.1926. |

### Example 117

### Example 117-A

### (R)-tert-Butyl 2-(2-((3′-(1-((tert-butoxycarbonyl)amino)butyl)-5-(hydroxymethyl)-[1,1′-biphenyl]-3-yl)methoxy)phenyl)acetate

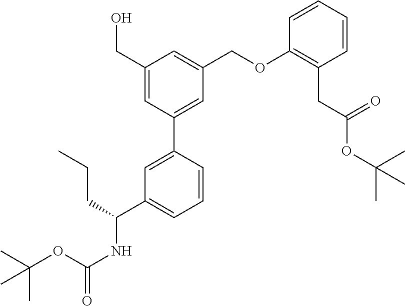 [SMILES: CCC[C@@H](NC(=O)OC(C)(C)C)c1cccc(-c2cc(CO)cc(COc3ccccc3CC(=O)OC(C)(C)C)c2)c1]

In a microwave vial, to a mixture of tert-butyl 2-(2-((3-bromo-5-(hydroxymethyl)benzyl)oxy)phenyl)acetate (Intermediate 59-A) (105 mg, 0.258 mmol) and (R)-tert-butyl (1-(3-(4,4,5,5-tetramethyl-1,3,2-dioxaborolan-2-yl)phenyl)butyl)carbamate (Intermediate 31) (102 mg, 0.271 mmol) in DMF (2.29 mL) and water (0.286 mL) was added 2M aqueous K₃PO₄ (0.258 mL, 0.516 mmol) and PdCl₂(dppf).CH₂Cl₂ adduct (CAS #95464-05-4) (8.4 mg, 0.010 mmol). The reaction was heated in a microwave at 110° C. for 60 minutes. The reaction was then diluted with water and EtOAc, the aq. layer was extracted with EtOAc, washed with water, dried over MgSO₄, filtered and concentrated. The crude product was purified by flash chromatography (0-100% EtOAc:Heptanes) to provide the title compound. MS (ESI+) m/z 576.3 (M+H).

### Example 117-B

### (R)-2-(2-((3′-(1-Aminobutyl)-5-(hydroxymethyl)-[1,1′-biphenyl]-3-yl)methoxy)phenyl)acetic acid

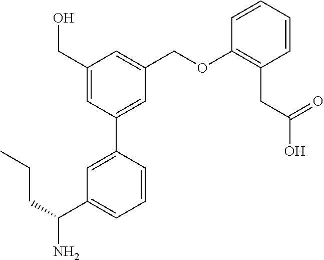 [SMILES: CCC[C@@H](N)c1cccc(-c2cc(CO)cc(COc3ccccc3CC(=O)O)c2)c1]

The title compound was synthesized as described in Example 20-B starting with (R)-tert-butyl 2-(2-((3′-(1-((tert-butoxycarbonyl)amino)butyl)-5-(hydroxymethyl)-[1,1′-biphenyl]-3-yl)methoxy)phenyl)acetate. ¹H NMR (400 MHz, METHANOL-d₄) δ ppm 8.16 (t, J=1.58 Hz, 1H) 7.99 (s, 1H) 7.65-7.75 (m, 1H) 7.61 (s, 1H) 7.45 (t, J=7.64 Hz, 1H) 7.35 (s, 1H) 7.28 (d, J=7.45 Hz, 1H) 7.11-7.22 (m, 2H) 6.96 (d, J=7.71 Hz, 1H) 6.87 (td, J=7.39, 1.01 Hz, 1H) 5.09-5.27 (m, 2H) 4.70 (s, 2H) 4.24 (dd, J=9.03, 6.13 Hz, 1H) 3.50-3.70 (m, 2H) 1.90-2.13 (m, 2H) 1.13-1.41 (m, 2H) 0.85-1.00 (m, 3H). HRMS calcd. for C₂₆H₂₉NO₄ (M+H)⁺ 420.2175. found 420.2169.

### Example 118

### (R)-2-(2-((3′-(1-Aminobutyl)-5-(methoxymethyl)-[1,1′-biphenyl]-3-yl)methoxy)phenyl)acetic acid

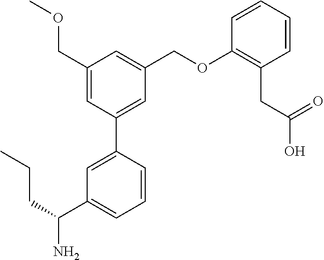 [SMILES: CCC[C@@H](N)c1cccc(-c2cc(COC)cc(COc3ccccc3CC(=O)O)c2)c1]

The title compound was synthesized as described in Examples 117-A and 20-B starting with tert-butyl 2-(2-((3-bromo-5-(methoxymethyl)benzyl)oxy)phenyl)acetate (Intermediate 61). ¹H NMR (400 MHz, METHANOL-d₄) δ ppm 8.18 (s, 1H) 8.02 (s, 1H) 7.65-7.73 (m, 1H) 7.58 (s, 1H) 7.45 (t, J=7.71 Hz, 1H) 7.34 (s, 1H) 7.28 (d, J=7.83 Hz, 1H) 7.12-7.22 (m, 2H) 6.96 (d, J=7.83 Hz, 1H) 6.87 (td, J=7.39, 1.01 Hz, 1H) 5.10-5.28 (m, 2H) 4.55 (s, 2H) 4.24 (dd, J=8.97, 6.19 Hz, 1H) 3.49-3.69 (m, 2H) 3.42 (s, 3H) 1.89-2.15 (m, 2H) 1.12-1.41 (m, 2H) 0.84-1.03 (m, 3H). HRMS calcd. for C₂₇H₃₁NO₄ (M+H)⁺ 434.2331. found 434.2349.

### Example 119

### Example 119-A

### tert-Butyl 2-(2-((3′-((R)-1-((tert-butoxycarbonyl)amino)ethyl)-5-(1-methoxyethyl)-[1,1′-biphenyl]-3-yl)methoxy)phenyl)acetate (mixture of diastereomers)

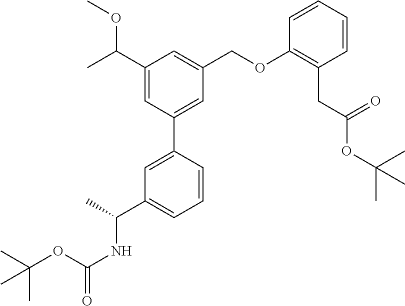 [SMILES: COC(C)c1cc(COc2ccccc2CC(=O)OC(C)(C)C)cc(-c2cccc([C@@H](C)NC(=O)OC(C)(C)C)c2)c1]

In a microwave vial, to a solution of (±)-tert-butyl 2-(2-((3-bromo-5-(1-methoxyethyl)benzyl)oxy)phenyl)acetate (Intermediate 68) (0.180 g, 0.413 mmol) and (R)-tert-butyl (1-(3-(4,4,5,5-tetramethyl-1,3,2-dioxaborolan-2-yl)phenyl)ethyl)carbamate (Intermediate 26) (0.151 g, 0.434 mmol) in DMF (3.76 mL) and water (0.376 mL) was added 2M aqueous K₃PO₄ (0.620 mL, 1.24 mmol) and PdCl₂(dppf).CH₂Cl₂ adduct (CAS #95464-05-4) (0.017 g, 0.021 mmol) and this was heated in the microwave at 110° C. for 1 hour. The reaction was diluted with water, saturated brine, saturated aqueous NH₄Cl and EtOAc. The layers were separated and the aq. layer was extracted with EtOAc. The combined organic layers were diluted with heptanes, washed with water, dried over MgSO₄, filtered and concentrated. The crude product was purified by flash chromatography (0-50% EtOAc:Heptanes) to provide the title compound. MS (ESI+) m/z 576.4 (M+H).

### Example 119-B

### 2-(2-((3′-((R)-1-Aminoethyl)-5-(1-methoxyethyl)-[1,1′-biphenyl]-3-yl)methoxy)phenyl)acetic acid (mixture of diastereomers)

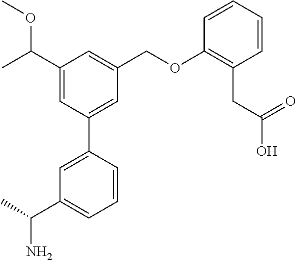 [SMILES: COC(C)c1cc(COc2ccccc2CC(=O)O)cc(-c2cccc([C@@H](C)N)c2)c1]

The title compound was synthesized as described in Example 114-B starting with tert-butyl 2-(2-((3′-((R)-1-((tert-butoxycarbonyl)amino)ethyl)-5-(1-methoxyethyl)-[1,1′-biphenyl]-3-yl)methoxy)phenyl)acetate. ¹H NMR (400 MHz, METHANOL-d₄) δ ppm 8.22 (s, 1H) 8.02 (s, 1H) 7.63-7.72 (m, 1H) 7.54 (s, 1H) 7.46 (t, J=7.71 Hz, 1H) 7.25-7.36 (m, 2H) 7.09-7.24 (m, 2H) 6.98 (d, J=8.21 Hz, 1H) 6.81-6.91 (m, 1H) 5.11-5.28 (m, 2H) 4.37-4.56 (m, 2H) 3.51-3.71 (m, 2H) 3.26 (d, J=0.88 Hz, 3H) 1.68 (d, J=6.95 Hz, 3H) 1.47 (d, J=6.44 Hz, 3H). HRMS calcd. for C₂₆H₂₉NO₄ (M+H)⁺ 420.2175. found 420.2155.

### Example 120

The following compounds were prepared with similar methods as described in Examples 119-A and 114-B using the aryl halides from Intermediates 52, 59-A, 61, 62, 65-B, 68 and 107-112 and the appropriate boronic ester from Intermediates 26, 27-B, 34-B, 105 and 106.

| | Structural/Chemical Name | ¹H NMR | HRMS |
|---|---|---|---|
| 120-A | 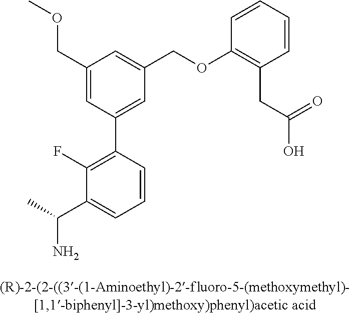 [SMILES: COCc1cc(COc2ccccc2CC(=O)O)cc(-c2cccc([C@@H](C)N)c2F)c1] | (400 MHz, DMSO-d₆) δ ppm 7.50- 7.63 (m, 2 H) 7.32-7.45 (m, 3 H) 7.15 -7.30 (m, 3 H) 7.03 (d, J = 7.83 Hz, 1 H) 6.84-6.92 (m, 1 H) 5.19 (s, 2 H) 4.49 (s, 2 H) 4.35 (q, J = 6.65 Hz, 1 H) 3.54 (s, 2 H) 3.33 (s, 3 H) 1.33 (d, J = 6.69 Hz, 3H). | calcd. for C₂₅H₂₆FNO₄ (M + H)⁺ 424.1924, found 424.1913. |
| 120-B | 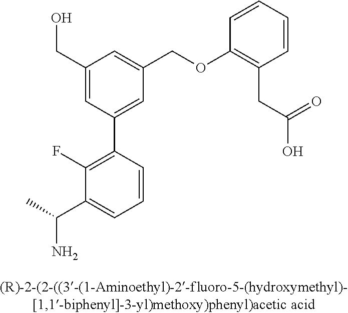 [SMILES: C[C@@H](N)c1cccc(-c2cc(CO)cc(COc3ccccc3CC(=O)O)c2)c1F] | (400 MHz, DMSO-d₆) δ ppm 7.49- 7.62 (m, 2 H) 7.39-7.46 (m, 2 H) 7.35 (td, J = 7.52, 1.77 Hz, 1 H) 7.13-7.30 (m, 3 H) 7.03 (d, J = 7.83 Hz, 1 H) 6.82-6.94 (m, 1 H) 5.18 (s, 2 H) 4.57 (s, 2 H) 4.35 (q, J = 6.69 Hz, 1 H) 3.53 (s, 2 H) 1.33 (d, J = 6.57 Hz, 3H). | calcd. for C₂₄H₂₄FNO₄ (M +H)⁺ 410.1768, found 410.1781. |
| 120-C | 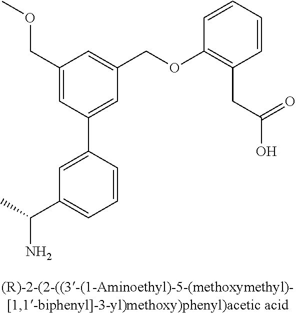 [SMILES: COCc1cc(COc2ccccc2CC(=O)O)cc(-c2cccc([C@@H](C)N)c2)c1] | (400 MHz, METHANOL-d₄) δ ppm 8.20 (s, 1 H) 8.04 (s, 1 H) 7.68 (d, J = 7.96 Hz, 1 H) 7.58 (s, 1 H) 7.45 (t, J = 7.71 Hz, 1 H) 7.28- 7.37 (m, 2 H) 7.13-7.23 (m, 2 H) 6.97 (d, J = 7.83 Hz, 1 H) 6.81- 6.93 (m, 1 H) 5.11-5.27 (m, 2 H) 4.55 (s, 2 H) 4.47 (q, J = 6.91 Hz, 1 H) 3.50-3.68 (m, 2 H) 3.42 (s, 3 H) 1.67 (d, J = 6.95 Hz, 3 H). | calcd. for C₂₅H₂₇NO₄ (M + H)⁺ 406.2018, found 406.2009. |
| 120-D | 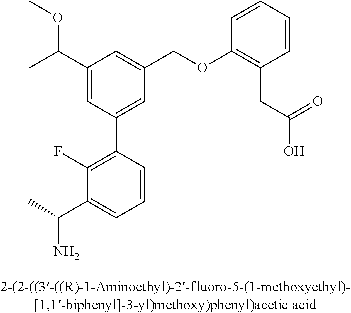 [SMILES: COC(C)c1cc(COc2ccccc2CC(=O)O)cc(-c2cccc([C@@H](C)N)c2F)c1] | (400 MHz, DMSO-d₆) δ ppm 7.50- 7.64 (m, 2 H) 7.31-7.45 (m, 3 H) 7.14-7.31 (m, 3 H) 7.05 (d, J = 7.83 Hz, 1 H) 6.89 (td, J = 7.36, 0.95 Hz, 1 H) 5.19 (s, 2 H) 4.29 - 4.44 (m, 2 H) 3.54 (s, 2H) 3.10- 3.23 (m, 3 H) 1.38 (d, J = 6.44 Hz, 3 H) 1.33 (d, J = 6.69 Hz, 3 H). | calcd. for C₂₆H₂₈FNO₄ (M + H)⁺ 438.2081, found 438.2064. |
| 120-E | 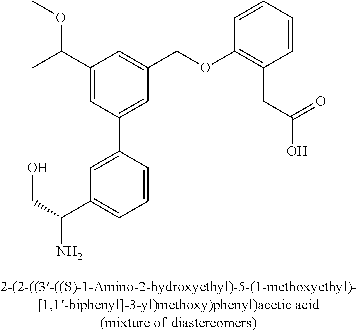 [SMILES: COC(C)c1cc(COc2ccccc2CC(=O)O)cc(-c2cccc([C@H](N)CO)c2)c1] | (400 MHz, METHANOL-d₄) δ ppm 8.13 (s, 1 H) 7.96 (s, 1 H) 7.67-7.76 (m, 1 H) 7.55 (s, 1 H) 7.48 (t, J = 7.77 Hz, 1 H) 7.29- 7.37 (m, 2H) 7.13-7.23 (m, 2H) 6.99 (d, J = 8.34 Hz, 1 H) 6.88 (td, J = 7.39, 1.01 Hz, 1 H) 5.13-5.27 (m, 2 H) 4.44 (q, J = 6.44 Hz, 1 H) 4.37 (dd, J = 8.65, 4.61 Hz, 1 H) 3.83-3.99 (m, 2 H) 3.61 (q, J = 16.04 Hz, 2 H) 3.26 (d, J = 0.88 Hz, 3 H) 1.46 (d, J = 6.44 Hz, 3 H). | calcd. for C₂₆H₂₉NO₅ (M + H)⁺ 436.2124, found 436.2100. |
| 120-F | 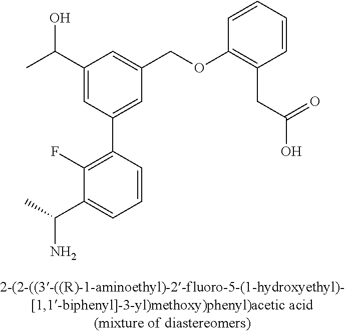 [SMILES: CC(O)c1cc(COc2ccccc2CC(=O)O)cc(-c2cccc([C@@H](C)N)c2F)c1] | (400 MHz, DMSO-d6) ppm 7.49-7.61 (m, 2 H) 7.44 (d, J = 8.08 Hz, 2 H) 7.36 (td, J = 7.48, 1.58 Hz, 1 H) 7.11-7.31 (m, 3 H) 7.04 (d, J = 7.96 Hz, 1 H) 6.82- 6.94 (m, 1 H) 5.17 (s, 2 H) 4.79 (q, J = 6.53 Hz, 1 H) 4.36 (q, J = 6.61 Hz, 1 H) 3.54 (s, 2 H) 1.23-1.45 (m, 6H). | calcd. for C25H26FNO4 (M + H)+ 424.1924, found 424.1937. |
| 120-G | 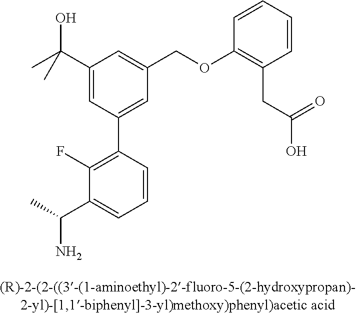 [SMILES: C[C@@H](N)c1cccc(-c2cc(COc3ccccc3CC(=O)O)cc(C(C)(C)O)c2)c1F] | (400 MHz, DMSO-d6 with ~5 μL TFA) ppm 8.35 (br. s., 3H) 7.63 (d, J = 1.39 Hz, 1 H) 7.50- 7.61 (m, 3 H) 7.46 (s, 1 H) 7.35- 7.44 (m, 1 H) 7.16- .31 (m, 2 H) 7.08 (d, J = 7.71 Hz, 1 H) 6.91 (td, J = 7.39, 0.88 Hz, 1 H) 5.18 (s, 2 H) 4.65-4.79 (m, 1 H) 3.59 (s, 2 H) 1.57 (d, J = 6.82 Hz, 3 H) 1.48 (s, 6 H). | calcd. for C26H28FNO4 (M + H)+ 438.2081, found 438.2090. |
| 120-H | 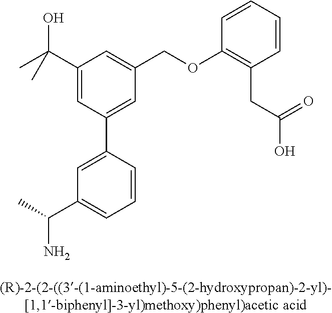 [SMILES: C[C@@H](N)c1cccc(-c2cc(COc3ccccc3CC(=O)O)cc(C(C)(C)O)c2)c1] | (400 MHz, DMSO-d6) ppm 8.33 (s, 1 H) 7.92 (s, 1 H) 7.71 (s, 1 H) 7.66 (d, J = 7.96 Hz, 1 H) 7.47 (s, 1 H) 7.42 (t, J = 7.64 Hz, 1 H) 7.28 (d, J = 7.71 Hz, 1 H) 7.04- 7.16 (m, 2 H) 6.94 (d, J = 7.83 Hz, 1 H) 6.75-6.89 (m, 1 H) 5.16 (s, 2 H) 4.27 (q, J = 6.69 Hz, 1 H) 3.29-3.46 (m, 2 H) 1.41-1.59 (m, 9 H). | calcd. for C26H29N04 (M + H)+ 420.2175, found 420.2191. |
| 120-I | 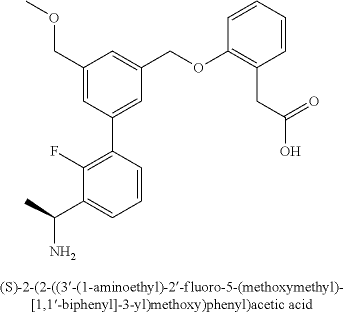 [SMILES: COCc1cc(COc2ccccc2CC(=O)O)cc(-c2cccc([C@H](C)N)c2F)c1] | (400 MHz, DMSO-d6) ppm 7.52- .62 (m, 2 H) 7.33-7.45 (m, 3 H) 7.15-7.30 (m, 3 H) 7.03 (d, J = 7.83 Hz, 1 H) 6.83-6.93 (m, 1 H) 5.19 (s, 2 H) 4.49 (s, 2 H) 4.36 (q, J = 6.61 Hz, 1 H) 3.54 (s, 2 H) 3.33 (s, 3 H) 1.34 (d, J = 6.69 Hz, 3 H). | calcd. for C25H26FNO4 (M + H)+ 424.1924, found 424.1932. |
| 120-J | 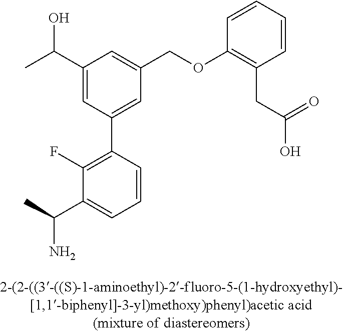 [SMILES: CC(O)c1cc(COc2ccccc2CC(=O)O)cc(-c2cccc([C@H](C)N)c2F)c1] | (400 MHz, DMSO-d6) ppm 7.48-7.60 (m, 2 H) 7.44 (d, J = 7.71 Hz, 2 H) 7.36 (td, J = 7.52, 1.64 Hz, 1 H) 7.14-7.30 (m, 3 H) 7.04 (d, J = 7.83 Hz, 1 H) 6.88 (td, J = 7.39, 1.01 Hz, 1 H) 5.17(s, 2 H) 4.79 (q, J = 6.40 Hz, 1 H) 4.36 (q, J = 6.74 Hz, 1 H) 3.54 (s, 2 H) 1.27-1.43 (m, 6 H). | calcd. for C25H26FNO4 (M + H)+ 424.1924, found 424.1923. |
| 120-K | 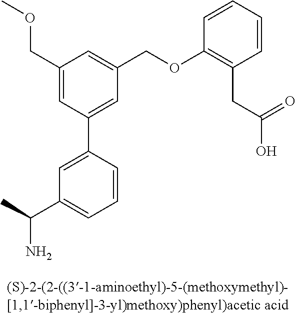 [SMILES: COCc1cc(COc2ccccc2CC(=O)O)cc(-c2cccc([C@H](C)N)c2)c1] | (400 MHz, METHANOL-d4) ppm 8.21 (t, J = 1.64 Hz, 1 H) 8.04 (s, 1 H) 7.68 (dt, J = 8.02, 1.23 Hz, 1 H) 7.58 (s, 1 H) 7.45 (t, J = 7.71 Hz, 1 H) 7.29-7.36 (m, 2H) 7.11- 7.21 (m, 2 H) 6.96 (d, J = 7.83 Hz, 1 H) 6.87 (td, J = 7.39, 1.01 Hz, 1 H) 5.11-5.27 (m, 2 H) 4.55 (s, 2 H) 4.47 (q, J = 6.91 Hz, 1 H) 3.50-3.69 (m, 2 H) 3.42 (s, 3 H) 1.67 (d, J = 6.95 Hz, 3H). | calcd. for C25H27NO4 (M + H)+ 406.2018, found 406.2004. |
| 120-L | 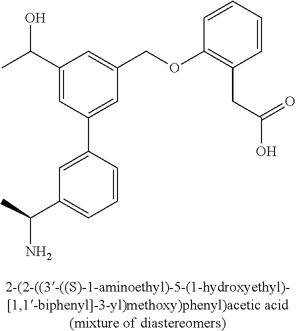 [SMILES: CC(O)c1cc(COc2ccccc2CC(=O)O)cc(-c2cccc([C@H](C)N)c2)c1] | (400 MHz, METHANOL-d4) ppm 8.19 (s, 1 H) 7.99 (s, 1 H) 7.70 (d, J = 8.08 Hz, 1 H) 7.62 (s, 1 H) 7.45 (t, J = 7.71 Hz, 1 H) 7.36 (s, 1 H) 7.32 (d, J = 7.58 Hz, 1 H) 7.12-7.23 (m, 2 H) 6.97 (d, J = 7.83 Hz, 1 H) 6.87 (td, J = 7.39, 1.01 Hz, 1 H) 5.12-5.27 (m, 2 H) 4.92 (q, J = 6.36 Hz, 1 H) 4.47 (q, J = 6.86 Hz, 1 H) 3.50-3.69 (m, 2 H) 1.67 (d, J = 6.95 Hz, 3 H) 1.50 (d, J = 6.44 Hz, 3 H). | calcd. for C25H27NO4 (M + H)+ 406.2018, found 406.2001. |
| 120-M | 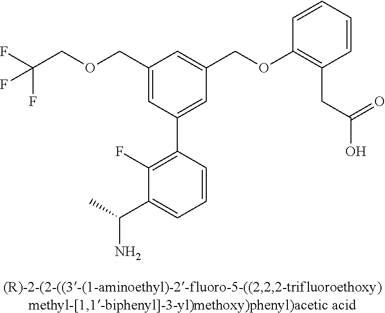 [SMILES: C[C@@H](N)c1cccc(-c2cc(COCC(F)(F)F)cc(COc3ccccc3CC(=O)O)c2)c1F] | (400 MHz, DMSO-d6) ppm 7.64 (s, 1 H) 7.57 (t, J = 6.51 Hz, 1 H) 7.44 (br. s., 2 H) 7.38 (td, J = 7.52, 1.64 Hz, 1 H) 7.23-7.32 (m, 1 H) 7.14-7.23 (m, 2 H) 7.04 (d, J = 7.83 Hz, 1 H) 6.84-6.93 (m, 1 H) 5.21 (s, 2H) 4.74 (s, 2 H) 4.37 (q, J = 6.65 Hz, 1 H) 4.14 (q, J = 9.43 Hz, 2 H) 3.54 (s, 2 H) 1.35 (d, J = 6.69 Hz, 3 H). | calcd. for C26H25F4NO4 (M + H)+ 492.1798, found 492.1787. |
| 120-N | 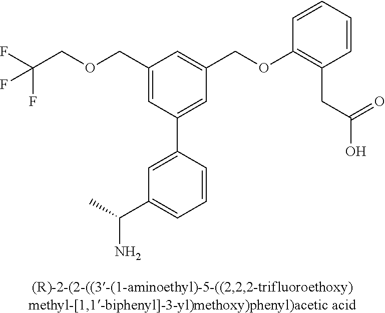 [SMILES: C[C@@H](N)c1cccc(-c2cc(COCC(F)(F)F)cc(COc3ccccc3CC(=O)O)c2)c1] | (400 MHz, METHANOL-d4) ppm 8.21 (t, J = 1.64 Hz, 1 H) 8.07 (s, 1 H) 7.65-7.72 (m, 1 H) 7.60 (s, 1 H) 7.46 (t, J = 7.71 Hz, 1 H) 7.30-7.39 (m, 2 H) 7.12-7.22 (m, 2 H) 6.97 (d, J = 7.83 Hz, 1 H) 6.87 (td, J = 7.36, 0.95 Hz, 1 H) 5.14-5.27 (m, 2 H) 4.77 (s, 2 H) 4.48 (q, J = 6.95 Hz, 1 H) 3.99 (q, J = 8.97 Hz, 2 H) 3.51-3.69 (m, 2 H) 1.68 (d, J = 6.82 Hz, 3 H). | calcd. for C26H26F3NO4 (M + H)+ 474.1892, found 474.1865. |
| 120-O | 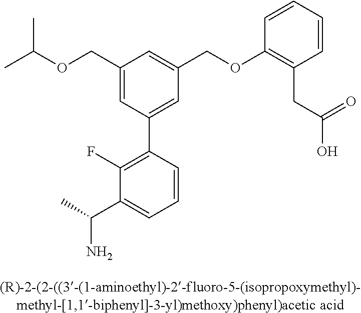 [SMILES: CC(C)OCc1cc(COc2ccccc2CC(=O)O)cc(-c2cccc([C@@H](C)N)c2F)c1] | (400 MHz, DMSO-d6) ppm 7.50-7.64 (m, 2H) 7.40 (s, 2 H) 7.36 (td, J = 7.55, 1.83 Hz, 1 H) 7.15-7.30 (m, 3 H) 7.03 (d, J = 7.96 Hz, 1 H) 6.82-6.93 (m, 1 H) 5.19 (s, 2 H) 4.53 (s, 2 H) 4.35 (q, J = 6.61 Hz, 1 H) 3.62-3.73 (m, 1 H) 3.53 (s, 2 H) 1.33 (d, J = 6.69 Hz, 3H) 1.16 (d, J = 6.06 Hz, 6 H). | calcd. for C27H30FNO4 (M + H)+ 452.2237, found 452.2228. |
| 120-P | 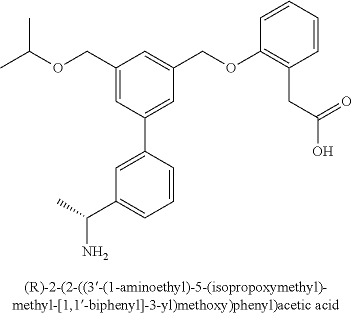 [SMILES: CC(C)OCc1cc(COc2ccccc2CC(=O)O)cc(-c2cccc([C@@H](C)N)c2)c1] | (400 MHz, METHANOL-d4) ppm 8.20 (t, J = 1.64 Hz, 1 H) 8.02 (s, 1 H) 7.64-7.71 (m, 1 H) 7.58 (s, 1 H) 7.45 (t, J = 7.71 Hz, 1 H) 7.27-7.37 (m, 2H) 7.11-7.22 (m, 2H) 6.96 (d, J = 7.83 Hz, 1 H) 6.87 (td, J = 7.39, 0.88 Hz, 1 H) 5.11-5.27 (m, 2 H) 4.61 (s, 2 H) 4.47 (q, J = 6.95 Hz, 1 H) 3.71- 3.84 (m, 1 H) 3.49-3.69 (m, 2 H) 1.67 (d, J = 6.95 Hz, 3 H) 1.24 (d, J = 6.06 Hz, 6 H). | calcd. for C27H31NO4 (M + H)+ 434.2331, found 434.2307. |
| 120-Q | 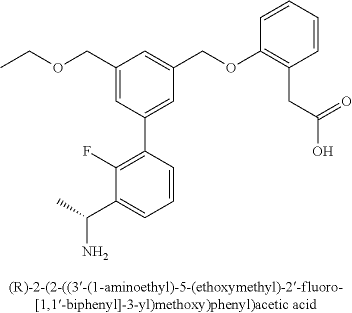 [SMILES: CCOCc1cc(COc2ccccc2CC(=O)O)cc(-c2cccc([C@@H](C)N)c2F)c1] | (400 MHz, DMSO-d6) ppm 7.50-7.64 (m, 2 H) 7.32-7.45 (m, 3 H) 7.14-7.30 (m, 3 H) 7.03 (d, J = 7.96 Hz, 1 H) 6.83-6.94 (m, 1 H) 5.19 (s, 2 H) 4.52 (s, 2 H) 4.35 (q, J = 6.61 Hz, 1 H) 3.49- 3.55 (m, 4 H) 1.33 (d, J = 6.69 Hz, 3H) 1.17 (t, J = 7.01 Hz, 3H). | calcd. for C26H28FNO4 (M + H)+ 438.2081, found 438.2090. |
| 120-R | 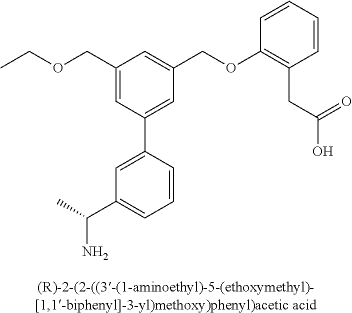 [SMILES: CCOCc1cc(COc2ccccc2CC(=O)O)cc(-c2cccc([C@@H](C)N)c2)c1] | (400 MHz, METHANOL-d4) ppm 8.21 (s, 1 H) 8.03 (s, 1 H) 7.68 (d, J = 7.96 Hz, 1 H) 7.58 (s, 1 H) 7.45 (t, J = 7.71 Hz, 1 H) 7.29- 7.37 (m, 2 H) 7.12-7.22 (m, 2 H) 6.97 (d, J = 7.96 Hz, 1 H) 6.83- 6.91 (m, 1 H) 5.11-5.28 (m, 2 H) 4.60 (s, 2 H) 4.47 (q, J = 6.91 Hz, 1 H) 3.50-3.68 (m, 4 H) 1.67 (d, J = 6.82 Hz, 3 H) 1.25 (t, J = 7.01 Hz, 3 H). | calcd. for C26H29NO4 (M + H)+ 420.2175, found 420.2166. |
| 120-S | 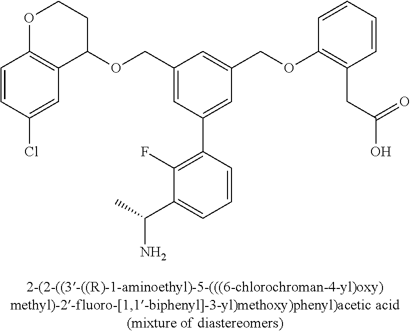 [SMILES: C[C@@H](N)c1cccc(-c2cc(COc3ccccc3CC(=O)O)cc(COC3CCOc4ccc(Cl)cc43)c2)c1F] | (400 MHz, DMSO-d6) ppm 7.52-7.64 (m, 2 H) 7.45-7.52 (m, 2 H) 7.30-7.41 (m, 2 H) 7.15- 7.30 (m, 4 H) 7.00-7.07 (m, 1 H) 6.84-6.92 (m, 1 H) 6.82 (d, J = 8.84 Hz, 1 H ) 5.20 (s, 2 H) 4.78 (d, J = 12.00 Hz, 1 H) 4.68 (d, J = 12.00 Hz, 1 H) 4.57 (t, J = 3.98 Hz, 1 H) 4.35 (q, J = 6.57 Hz, 1 H) 4.11-4.29 (m, 2 H) 3.54 (s, 2 H) 2.09-2.21 (m, 1 H) 1.94-2.09 (m, 1 H) 1.33 (d, J = 6.69 Hz, 3 H). | calcd. for C33H31CIFNO5 (M + H)+ 576.1953, found 576.1965. |
| 120-T | 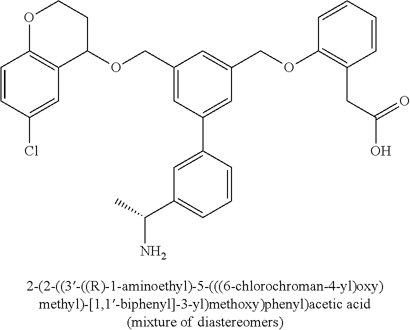 [SMILES: C[C@@H](N)c1cccc(-c2cc(COc3ccccc3CC(=O)O)cc(COC3CCOc4ccc(Cl)cc43)c2)c1] | (400 MHz. DMSO-d6) + 5 μL TFA) ppm 8.26 (br. s., 3 H) 7.82 (s, 1 H) 7.67-7.76 (m, 2 H) 7.64 (s, 1 H) 7.43-7.59 (m, 3 H) 7.34 (d, J = 2.65 Hz, 1 H) 7.17- 7.28 (m, 3 H) 7.06 (d, J = 7.83 Hz, 1 H) 6.91 (t, J = 6.88 Hz, 1 H) 6.83 (d, J = 8.72 Hz, 1 H) 5.22 (s, 2 H) 4.80 (d, J = 11.75 Hz, 1 H) 4.69 (d, J = 11.75 Hz, 1 H) 4.59 (t, J = 3.92 Hz, 1 H) 4.43-4.56 (m, 1 H) 4.11- 4.31 (m, 2H) 3.61 (s, 2 H) 2.10- 2.22 (m, 1 H) 1.97-2.10 (m, 1 H) 1.56 (d, J = 6.82 Hz, 3H). | calcd. for C33H32ClNO5 (M + H)+ 558.2047, found 558.2072. |
| 120-U | 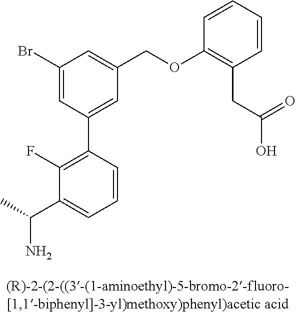 [SMILES: C[C@@H](N)c1cccc(-c2cc(Br)cc(COc3ccccc3CC(=O)O)c2)c1F] | (400 MHz, DMSO-d6) δ 7.72- 7.54 (m, 4H), 7.40 (td, J = 7.6, 1.8 Hz, 1H), 7.31-7.16 (m, 3H), 7.03-6.96 (m, 1H), 6.89 (t, J = 7.2 Hz, 1H), 5.22 (s, 2H), 4.35 (q, J = 6.6 Hz, 1H), 3.54 (d, J = 1.3 Hz, 2H), 3.40 (d, J = 7.5 Hz, 5H), 1.34 (d, J = 6.6 Hz, 3H). | calcd. for C23H21BrFNO3 (M + H)+ 458.0767 and 460.0747, found 458.0773 and 460.0758. |
| 120-V | 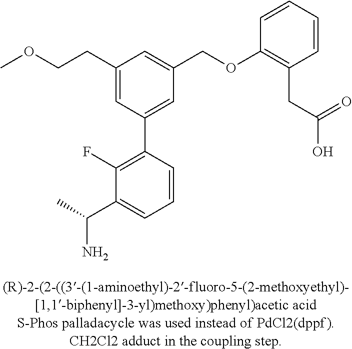 [SMILES: COCCc1cc(COc2ccccc2CC(=O)O)cc(-c2cccc([C@@H](C)N)c2F)c1] | (400 MHz, DMSO-d6) ppm 7.51-7.59 (m, 1 H) 7.49 (s, 1 H) 7.29-7.41 (m, 3 H) 7.15-7.29 (m, 3 H) 7.03 (d, J = 7.83 Hz, 1 H) 6.82-6.94 (m, 1 H) 5.15 (s, 2H) 4.35 (q, J = 6.57 Hz, 1 H) 3.59 (t, J = 6.82 Hz, 2 H) 3.54 (s, 2 H) 3.25 (s, 3 H) 2.88 (t, J = 6.82 Hz, 2 H) 1.33 (d, J = 6.57 Hz, 3 H). | calcd. for C26H28FNO4 (M + H)+ 438.2081, found 438.2065. |
| 120-W | 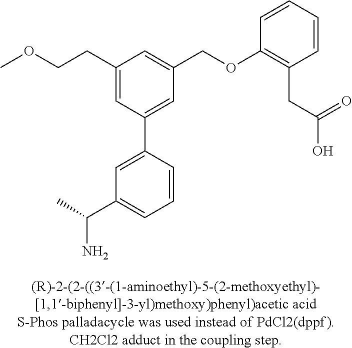 [SMILES: COCCc1cc(COc2ccccc2CC(=O)O)cc(-c2cccc([C@@H](C)N)c2)c1] | (400 MHz, METHANOL-d4) ppm 8.18 (t, J = 1.64 Hz, 1 H) 7.94 (s, 1 H) 7.63-7.71 (m, 1 H) 7.38- 7.50 (m, 2 H) 7.30 (d, J = 7.71 Hz, 1 H) 7.23 (s, 1 H) 7.10-7.21 (m, 2 H) 6.96 (d, J = 7.71 Hz, 1 H) 6.87 (td, J = 7.39, 1.01 Hz, 1 H) 5.07-5.24 (m, 2 H) 4.46 (q, J = 6.82 Hz, 1 H) 3.50-3.73 (m, 4 H) 3.36 (s, 3 H) 2.95 (t, J = 6.88 Hz, 2 H) 1.66 (d, J = 6.82 Hz, 3 H). | calcd. for C26H29NO4 (M + H)+ 420.2175, found 420.2153. |
| 120-X | 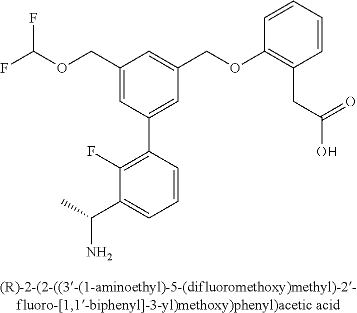 [SMILES: C[C@@H](N)c1cccc(-c2cc(COc3ccccc3CC(=O)O)cc(COC(F)F)c2)c1F] | (400 MHz, DMSO-d6) δ 7.66 (d, J = 2.0 Hz, 1H), 7.57 (td, J = 7.4, 1.8 Hz, 1H), 7.51-7.45 (m, 2H), 7.38 (td, J = 7.5, 1.8 Hz, 1H), 7.27 (t, J = 7.6 Hz, 1H), 7.23- 7.16 (m, 2H), 7.06-7.02 (m, 1H), 6.88 (td, J = 7.4, 1.1 Hz, 1H), 6.82 (t, J = 75.6 Hz, 1H), 5.21 (s, 2H), 4.99 (s, 2H), 4.36 (q, J = 6.6 Hz, 1H), 3.54 (s, 2H), 1.34 (d, J = 6.7 Hz, 3H). | calcd. for C25H24F3NO4 (M + H)+ 460.1736, found 460.1711. |

### Example 121

### Example 121-A

### (±)-tert-Butyl 2-(2-((3-(1-methoxyethyl)-5-(4,4,5,5-tetramethyl-1,3,2-dioxaborolan-2-yl)benzyl)oxy)phenyl)acetate

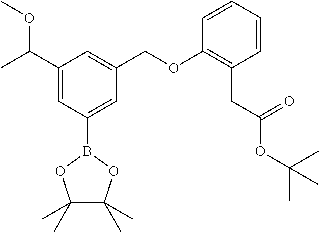 [SMILES: COC(C)c1cc(COc2ccccc2CC(=O)OC(C)(C)C)cc(B2OC(C)(C)C(C)(C)O2)c1]

To a solution of (±)-tert-butyl 2-(2-((3-bromo-5-(1-methoxyethyl)benzyl)oxy)phenyl)acetate (Intermediate 68) (0.289 g, 0.664 mmol) in DMF (6.64 mL), bis(pinacolato)diboron (CAS #73183-34-3) (0.253 g, 0.996 mmol), potassium acetate (0.195 g, 1.99 mmol), and PdCl₂(dppf).CH₂Cl₂ adduct (CAS #95464-05-4) (0.027 g, 0.033 mmol) were added and the reaction heated to 110° C. After 2 hours the reaction was cooled, quenched with water, extracted twice with EtOAc, diluted with heptanes, washed with water, dried with MgSO₄, filtered and concentrated. The crude product was purified by flash column chromatography (0-50% EtOAc:Heptanes) to provide the title compound. MS (ESI+) m/z 505.3 (M+Na).

### Example 121-B

### tert-Butyl 2-(2-((3′-((S)-1-amino-2-fluoroethyl)-5-(1-methoxyethyl)-[1,1′-biphenyl]-3-yl)methoxy)phenyl)acetate (mixture of diastereomers)

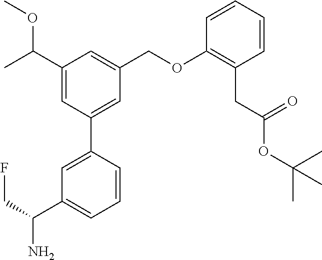 [SMILES: COC(C)c1cc(COc2ccccc2CC(=O)OC(C)(C)C)cc(-c2cccc([C@H](N)CF)c2)c1]

The title compound was synthesized as described in Example 119-A starting with tert-butyl 2-(2-((3-(1-methoxyethyl)-5-(4,4,5,5-tetramethyl-1,3,2-dioxaborolan-2-yl)benzyl)oxy)phenyl)acetate and (S)-1-(3-bromophenyl)-2-fluoroethanamine (CAS #1386462-26-5). MS (ESI+) m/z 494.3 (M+H).

### Example 121-C

### 2-(2-((3′-((S)-1-Amino-2-fluoroethyl)-5-(1-methoxyethyl)-[1,1′-biphenyl]-3-yl)methoxy)phenyl)acetic acid (mixture of diastereomers)

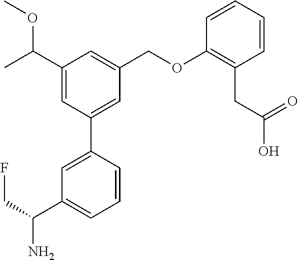 [SMILES: COC(C)c1cc(COc2ccccc2CC(=O)O)cc(-c2cccc([C@H](N)CF)c2)c1]

The title compound was synthesized as described in Example 114-B starting with tert-butyl 2-(2-((3′-((S)-1-amino-2-fluoroethyl)-5-(1-methoxyethyl)-[1,1′-biphenyl]-3-yl)methoxy)phenyl)acetate. ¹H NMR (400 MHz, DMSO-d₆ with ˜5 uL TFA) δ ppm 8.71 (br. s., 3H) 7.86 (s, 1H) 7.77 (d, J=7.96 Hz, 1H) 7.69 (s, 1H) 7.53-7.62 (m, 2H) 7.47-7.53 (m, 1H) 7.43 (s, 1H) 7.18-7.29 (m, 2H) 7.07 (d, J=7.83 Hz, 1H) 6.85-6.98 (m, 1H) 5.20 (s, 2H) 4.68-4.91 (m, 3H) 4.41 (q, J=6.32 Hz, 1H) 3.60 (s, 2H) 3.18 (s, 3H) 1.41 (d, J=6.44 Hz, 3H). HRMS calcd. for C₂₆H₂₈FNO₄ (M+H)⁺ 438.2081. found 438.2079.

### Example 122

### (S)-2-(2-((3′-(1-Amino-2-hydroxyethyl)-6-fluoro-[1,1′-biphenyl]-3-yl)methoxy)phenyl)acetic acid

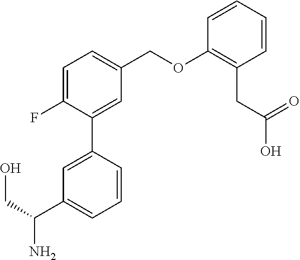 [SMILES: N[C@H](CO)c1cccc(-c2cc(COc3ccccc3CC(=O)O)ccc2F)c1]

The title compound as synthesized as in Example 75, using methyl 2-(2-((3-bromo-4-fluorobenzyl)oxy)phenyl)acetate (Intermediate 69) and (S)-tert-butyl (2-hydroxy-1-(3-(4,4,5,5-tetramethyl-1,3,2-dioxaborolan-2-yl)phenyl)ethyl)carbamate (Intermediate 34-B). ¹H NMR (600 MHz, DMSO-d₆) δ ppm 8.07 (s, 1H) 7.82-7.87 (m, 1H) 7.63 (d, J=3.76 Hz, 1H) 7.45-7.49 (m, 1H) 7.37-7.43 (m, 1H) 7.35 (d, J=7.34 Hz, 1H) 7.29 (dd, J=11.10, 8.34 Hz, 1H) 7.06-7.22 (m, 2H) 6.96 (d, J=7.89 Hz, 1H) 6.81 (t, J=7.24 Hz, 1H) 5.15 (s, 2H) 4.17 (dd, J=6.88, 4.40 Hz, 1H) 3.65-3.77 (m, 2H) 3.29-3.47 (m, 2H). HRMS calcd. for C₂₃H₂₂FNO₄ (M+H)⁺ 396.1566. found 396.1592.

### Example 123

### (S)-2-(2-((3′-(1-Amino-2-hydroxyethyl)-2-fluoro-[1,1′-biphenyl]-3-yl)methoxy)phenyl)acetic acid

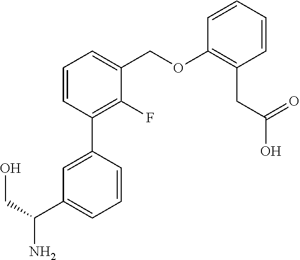 [SMILES: N[C@H](CO)c1cccc(-c2cccc(COc3ccccc3CC(=O)O)c2F)c1]

The title compound was synthesized as described in Example 122 starting with methyl 2-(2-((3-bromo-2-fluorobenzyl)oxy)phenyl)acetate (Intermediate 70) and (S)-tert-butyl (2-hydroxy-1-(3-(4,4,5,5-tetramethyl-1,3,2-dioxaborolan-2-yl)phenyl)ethyl)carbamate (Intermediate 34-B). ¹H NMR (400 MHz, METHANOL-d₄) δ ppm 7.52-7.74 (m, 4H) 7.42-7.50 (m, 2H) 7.18-7.34 (m, 3H) 7.05 (d, J=7.71 Hz, 1H) 6.94 (td, J=7.45, 1.01 Hz, 1H) 5.24 (s, 2H) 4.43 (dd, J=8.08, 4.29 Hz, 1H) 3.91-4.00 (m, 1H) 3.80-3.89 (m, 1H) 3.65 (s, 2H). HRMS calcd. for C₂₃H₂₂FNO₄ (M+H)⁺ 396.1611. found 396.1615.

### Example 124

### (R)-2-(2-((3′-(1-Aminoethyl)-6-fluoro-[1,1′-biphenyl]-3-yl)methoxy)phenyl)acetic acid

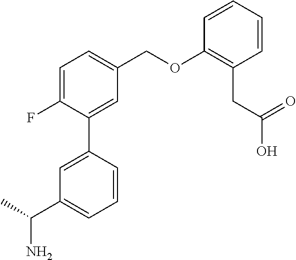 [SMILES: C[C@@H](N)c1cccc(-c2cc(COc3ccccc3CC(=O)O)ccc2F)c1]

The title compound was synthesized as described in Example 122 starting with methyl 2-(2-((3-bromo-4-fluorobenzyl)oxy)phenyl)acetate (Intermediate 69) and (R)-tert-butyl (1-(3-(4,4,5,5-tetramethyl-1,3,2-dioxaborolan-2-yl)phenyl)ethyl)carbamate (Intermediate 26). ¹H NMR (400 MHz, METHANOL-d₄) δ ppm 8.05 (s, 1H) 7.94 (dd, J=7.77, 2.08 Hz, 1H) 7.70 (dt, J=7.77, 1.42 Hz, 1H) 7.41-7.47 (m, 2H) 7.33-7.40 (m, 1H) 7.12-7.21 (m, 3H) 6.97 (d, J=7.83 Hz, 1H) 6.83-6.90 (m, 1H) 5.01-5.25 (m, 2H) 4.48 (q, J=6.91 Hz, 1H) 3.45-3.64 (m, 2H) 1.68 (d, J=6.95 Hz, 3H). HRMS calcd. for C₂₃H₂₂FNO₃ (M+H)⁺ 380.1662. found 380.1653.

### Example 125

### (R)-2-(2-((3′-(1-Aminoethyl)-2-fluoro-[1,1′-biphenyl]-3-yl)methoxy)phenyl)acetic acid

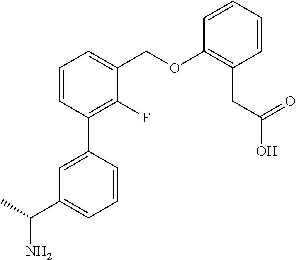 [SMILES: C[C@@H](N)c1cccc(-c2cccc(COc3ccccc3CC(=O)O)c2F)c1]

The title compound was synthesized as described in Example 122 starting with methyl 2-(2-((3-bromo-2-fluorobenzyl)oxy)phenyl)acetate (Intermediate 70) and (R)-tert-butyl (1-(3-(4,4,5,5-tetramethyl-1,3,2-dioxaborolan-2-yl)phenyl)ethyl)carbamate (Intermediate 26). ¹H NMR (400 MHz, METHANOL-d₄) δ ppm 7.67 (br s, 1H) 7.54-7.65 (m, 3H) 7.43-7.52 (m, 2H) 7.19-7.33 (m, 3H) 7.06 (d, J=7.96 Hz, 1H) 6.91-6.98 (m, 1H) 5.24 (s, 2H) 4.54 (q, J=6.78 Hz, 1H) 3.65 (s, 2H) 1.68 (d, J=6.82 Hz, 3H). HRMS calcd. for C₂₃H₂₂FNO₃ (M+H)⁺ 380.1662. found 380.1656.

### Example 126

### Example 126-A

### Methyl 2-(2-((7-(3-(aminomethyl)phenyl)benzo[d][1,3]dioxol-5-yl)methoxy)phenyl)acetate

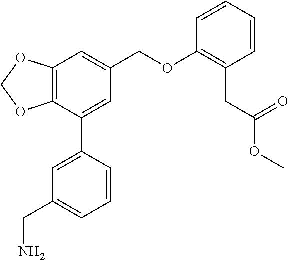 [SMILES: COC(=O)Cc1ccccc1OCc1cc2c(c(-c3cccc(CN)c3)c1)OCO2]

To a solution of methyl 2-(2-((7-bromobenzo[d][1,3]dioxol-5-yl)methoxy)phenyl)acetate (Intermediate 71) (70 mg, 0.185 mmol) and 3-aminomethylphenylboronic acid hydrochloride salt (CAS #146285-80-5) (52 mg, 0.277 mmol) in DMF (2.0 mL)/water (0.22 mL) was added a 2.0M aq. solution of K₃PO₄ (0.37 mL, 0.738 mmol); this mixture was degassed for 10 minutes with N₂(g), and then PdCl₂(dppf).CH₂Cl₂ adduct (CAS #95464-05-4) (15.0 mg, 0.018 mmol) was added. The reaction was sealed and heated at 110° C. in an oil bath for 1 hr. The reaction was cooled to room temperature, filtered over a plug of Celite and the filtrate liquor was partitioned between EtOAc/H₂O and the layers separated; the organic phase was washed with brine; dried over Na₂SO₄, and concentrated in vacuo to provide the title compound which was used without further purification. MS (ESI+) m/z 406.1 (M+H).

### Example 126-B

### 2-(2-((7-(3-(Aminomethyl)phenyl)benzo[d][1,3]dioxol-5-yl)methoxy)phenyl)acetic acid

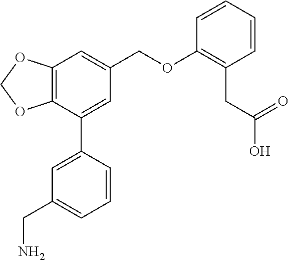 [SMILES: NCc1cccc(-c2cc(COc3ccccc3CC(=O)O)cc3c2OCO3)c1]

Methyl 2-(2-((7-(3-(aminomethyl)phenyl)benzo[d][1,3]dioxol-5-yl)methoxy)phenyl)acetate (50 mg, 0.123 mmol) was dissolved in MeOH (1.0 mL) and 2.0 M LiOH solution (0.245 mL, 0.491 mmol) was added and this mixture was heated at 60° C. in an oil bath for 2 hr. The reaction mixture was cooled to room temperature and concentrated in vacuo. The resulting residue was purified by preparative HPLC (Method B) to afford the title compound. ¹H NMR (600 MHz, METHANOL-d₄) δ ppm 7.93 (s, 1H) 7.89 (d, J=7.61 Hz, 1H) 7.49 (t, J=7.75 Hz, 1H) 7.39 (d, J=7.70 Hz, 1H) 7.32 (s, 1H) 7.18-7.24 (m, 2H) 6.99 (d, J=8.07 Hz, 1H) 6.94 (d, J=1.47 Hz, 1H) 6.91 (t, J=7.38 Hz, 1H) 6.05 (s, 2H) 5.10 (s, 2H) 4.18 (s, 2H) 3.65 (s, 2H). HRMS calcd. for C₂₃H₂₁NO₅ (M+H)⁺ 392.1498. found 392.1489.

### Example 127

### Example 127-A

### Methyl 2-(2-((4-methoxy-3-(4,4,5,5-tetramethyl-1,3,2-dioxaborolan-2-yl)benzyl)oxy)phenyl)acetate

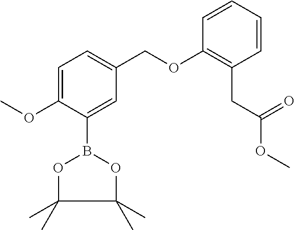

To a solution of methyl 2-(2-((3-bromo-4-methoxybenzyl)oxy)phenyl)acetate (Intermediate 72) (352 mg, 0.964 mmol) and 4,4,4′,4′,5,5,5′,5′-octamethyl-2,2′-bi(1,3,2-dioxaborolane) (CAS #73183-34-3) (306 mg, 1.205 mmol) in dioxane (4.5 mL) was added KOAc (284 mg, 2.89 mmol); this mixture was degassed for 10 minutes with N₂(g), and then PdCl₂(dppf).CH₂Cl₂ adduct (CAS #95464-05-4) (79 mg, 0.096 mmol) was added. The reaction was sealed and heated at 110° C. in an oil bath for 8 hr. The reaction was cooled to room temperature, filtered over a plug of Celite and the filtrate liquor was partitioned between EtOAc/H₂O and the layers separated; the organic phase was washed with brine, dried over Na₂SO₄, and concentrated in vacuo. The residue was purified by flash column chromatography on silica gel (heptanes/EtOAc=100:0 to 50:50) to afford the title compound. ¹H NMR (400 MHz, CHLOROFORM-d) δ ppm 7.60 (d, J=2.15 Hz, 1H) 7.39 (dd, J=8.53, 2.34 Hz, 1H) 7.08-7.17 (m, 2H) 6.84-6.88 (m, 2H) 6.80 (d, J=8.59 Hz, 1H) 4.93 (s, 2H) 3.77 (s, 3H) 3.58 (s, 2H) 3.53-3.57 (m, 3H) 1.22-1.35 (m, 12H).

### Example 127-B

### (S)-2-(2-((3′-(1-Amino-2-hydroxyethyl)-6-methoxy-[1,1′-biphenyl]-3-yl)methoxy)phenyl)acetic acid

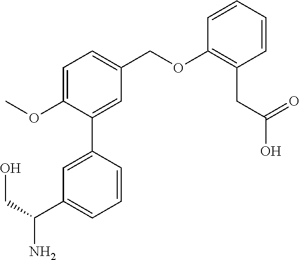 [SMILES: COc1ccc(COc2ccccc2CC(=O)O)cc1-c1cccc([C@H](N)CO)c1]

The title compound was synthesized as described in Example 126 starting with (S)-methyl 2-(2-((3′-(1-amino-2-hydroxyethyl)-6-methoxy-[1,1′-biphenyl]-3-yl)methoxy)phenyl)acetate (CAS #209963-05-3). ¹H NMR (400 MHz, METHANOL-d₄) δ ppm 7.59-7.68 (m, 2H) 7.45-7.49 (m, 2H) 7.40-7.44 (m, 1H) 7.33-7.38 (m, 1H) 7.16-7.26 (m, 2H) 7.09 (d, J=8.46 Hz, 1H) 7.02 (d, J=7.71 Hz, 1H) 6.86-6.93 (m, 1H) 5.09 (s, 2H) 4.38 (dd, J=8.46, 4.42 Hz, 1H) 3.85-4.01 (m, 2H) 3.81 (s, 3H) 3.62 (s, 2H). HRMS calcd. for C₂₄H₂₅NO₅ (M+H)⁺ 408.1811. found 408.1810.

### Example 128

### Example 128-A

### tert-Butyl 2-(2-((3-chloro-5-((cyclopropylmethyl)amino)benzyl)oxy)phenyl)acetate

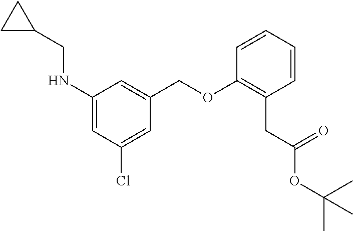 [SMILES: CC(C)(C)OC(=O)Cc1ccccc1OCc1cc(Cl)cc(NCC2CC2)c1]

To a solution of tert-butyl 2-(2-((3-bromo-5-chlorobenzyl)oxy)phenyl)acetate (Intermediate 53) (400 mg, 0.972 mmol) and cyclopropylmethanamine (104 mg, 1.457 mmol) in acetonitrile (5.0 mL) was added cesium carbonate (950 mg, 2.91 mmol); this mixture was degassed for 10 minutes with N₂(g), and then BrettPhos palladacycle (39.0 mg, 0.049 mmol) was added. The reaction was sealed and heated at 110° C. in an oil bath for 1 hr. The reaction was cooled to room temperature, filtered over a plug of Celite and the filtrate liquor was partitioned between EtOAc/H₂O and the layers separated; the organic phase was washed with brine, dried over Na₂SO₄, and concentrated in vacuo. The residue was purified by flash column chromatography on silica gel (heptanes/EtOAc=100:0 to 60:40) to afford the title compound. MS (ESI+) m/z 402.1 (M+H).

### Example 128-B

### (S)-tert-Butyl 2-(2-((3′-(1-((tert-butoxycarbonyl)amino)-2-hydroxyethyl)-5-((cyclopropylmethyl)amino)-[1,1′-biphenyl]-3-yl)methoxy)phenyl)acetate

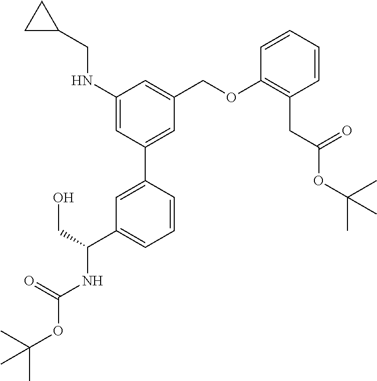 [SMILES: CC(C)(C)OC(=O)Cc1ccccc1OCc1cc(NCC2CC2)cc(-c2cccc([C@@H](CO)NC(=O)OC(C)(C)C)c2)c1]

To a solution of tert-butyl 2-(2-((3-chloro-5-((cyclopropylmethyl)amino)benzyl)oxy)phenyl)acetate (100 mg, 0.249 mmol) and (S)-tert-butyl (2-hydroxy-1-(3-(4,4,5,5-tetramethyl-1,3,2-dioxaborolan-2-yl)phenyl)ethyl)carbamate (Intermediate 34-B) (136 mg, 0.373 mmol) in acetonitrile (2.0 mL) was added a 2.0M aq. solution of K₃PO₄ (0.622 mL, 1.244 mmol); this mixture was degassed for 10 minutes with N₂(g), and then Sphos palladacycle (8.4 mg, 0.012 mmol) was added. The reaction was sealed and heated at 110° C. in an oil bath for 1 hr. The reaction was cooled to room temperature, filtered over a plug of Celite and the filtrate liquor was partitioned between EtOAc/H₂O and the layers separated; the organic phase was washed with brine, dried over Na₂SO₄, and concentrated in vacuo. The residue was purified by flash column chromatography on silica gel (heptanes/EtOAc=100:0 to 70:30) to afford the title compound. MS (ESI+) m/z 603.4 (M+H).

### Example 128-C

### (S)-2-(2-((3′-(1-Amino-2-hydroxyethyl)-5-((cyclopropylmethyl)amino)-[1,1′-biphenyl]-3-yl)methoxy)phenyl)acetic acid

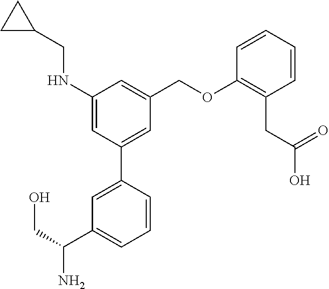 [SMILES: N[C@H](CO)c1cccc(-c2cc(COc3ccccc3CC(=O)O)cc(NCC3CC3)c2)c1]

The title compound was synthesized as described in Example 108-B starting with (S)-tert-butyl 2-(2-((3′-(1-((tert-butoxycarbonyl)amino)-2-hydroxyethyl)-5-((cyclopropylmethyl)amino)-[1,1′-biphenyl]-3-yl)methoxy)phenyl)acetate. ¹H NMR (400 MHz, METHANOL-d₄) δ ppm 8.11 (s, 1H) 7.65 (d, J=8.08 Hz, 1H) 7.43 (t, J=7.71 Hz, 1H) 7.36 (s, 1H) 7.28 (d, J=7.45 Hz, 1H) 7.10-7.20 (m, 2H) 6.94 (d, J=7.58 Hz, 1H) 6.82-6.89 (m, 2H) 6.67 (s, 1H) 4.99-5.23 (m, 2H) 4.33 (dd, J=8.97, 4.55 Hz, 1H) 3.79-4.04 (m, 2H) 3.44-3.72 (m, 2H) 3.03 (d, J=6.69 Hz, 2H) 1.12 (m, 1H) 0.45-0.62 (m, 2H) 0.20-0.35 (m, 2H). HRMS calcd. for C₂₇H₃₀N₂O₄ (M+H)⁺ 447.2284. found 447.2285.

### Example 129

### (S)-2-(2-((3′-(1-Amino-2-hydroxyethyl)-5-(ethylamino)-[1,1′-biphenyl]-3-yl)methoxy)phenyl)acetic acid

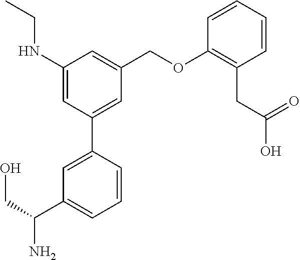 [SMILES: CCNc1cc(COc2ccccc2CC(=O)O)cc(-c2cccc([C@H](N)CO)c2)c1]

The title compound was synthesized as described in Example 128 using ethylamine. ¹H NMR (600 MHz, METHANOL-d₄) δ ppm 8.11 (s, 1H) 7.65 (d, J=7.98 Hz, 1H) 7.42 (t, J=7.70 Hz, 1H) 7.36 (s, 1H) 7.28 (d, J=7.52 Hz, 1H) 7.11-7.19 (m, 2H) 6.94 (d, J=7.79 Hz, 1H) 6.82-6.88 (m, 2H) 6.66 (s, 1H) 5.01-5.20 (m, 2H) 4.32 (dd, J=8.99, 4.49 Hz, 1H) 3.79-4.01 (m, 2H) 3.49-3.69 (m, 2H) 3.20 (q, J=7.15 Hz, 2H) 1.27 (t, J=7.15 Hz, 3H). HRMS calcd. for C₂₅H₂₈N₂O₄ (M+H)⁺ 421.2113. found 421.2117.

### Example 130

### (S)-2-(2-((3′-(1-Amino-2-hydroxyethyl)-5-(isopropylamino)-[1,1′-biphenyl]-3-yl)methoxy)phenyl)acetic acid

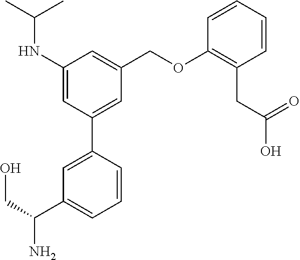 [SMILES: CC(C)Nc1cc(COc2ccccc2CC(=O)O)cc(-c2cccc([C@H](N)CO)c2)c1]

The title compound was synthesized as described in Example 128 using isopropylamine. ¹H NMR (400 MHz, METHANOL-d₄) δ ppm 7.80 (s, 1H) 7.72-7.77 (m, 1H) 7.64 (s, 1H) 7.57 (t, J=7.71 Hz, 1H) 7.45-7.50 (m, 1H) 7.37 (s, 1H) 7.29 (s, 1H) 7.20-7.26 (m, 2H) 7.00 (d, J=7.83 Hz, 1H) 6.94 (td, J=7.45, 1.01 Hz, 1H) 5.25 (s, 2H) 4.45 (dd, J=8.02, 4.36 Hz, 1H) 3.77-4.04 (m, 3H) 3.71 (s, 2H) 1.32 (d, J=6.44 Hz, 6H). HRMS calcd. for C₂₆H₃₀N₂O₄ (M+H)⁺ 435.2239. found 435.2270.

### Example 131

### (S)-2-(2-((3′-(1-Amino-2-hydroxyethyl)-5-(methylamino)-[1,1′-biphenyl]-3-yl)methoxy)phenyl)acetic acid

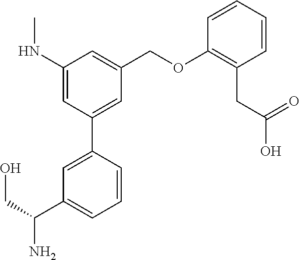 [SMILES: CNc1cc(COc2ccccc2CC(=O)O)cc(-c2cccc([C@H](N)CO)c2)c1]

The title compound was synthesized as described in Example 128 using methylamine. ¹H NMR (400 MHz, METHANOL-d₄) δ ppm 7.66-7.84 (m, 2H) 7.53 (t, J=7.71 Hz, 1H) 7.42 (d, J=7.71 Hz, 1H) 7.31 (s, 1H) 7.18-7.28 (m, 2H) 7.08 (s, 1H) 6.98-7.03 (m, 2H) 6.92 (td, J=7.42, 0.95 Hz, 1H) 5.18 (s, 2H) 4.43 (dd, J=8.21, 4.29 Hz, 1H) 3.82-4.02 (m, 2H) 3.70 (s, 2H) 2.94 (s, 3H). HRMS calcd. for C₂₄H₂₆N₂O₄ (M+H)⁺ 407.1926. found 407.1956.

### Example 132

### (S)-2-(2-((3′-(1-Amino-2-hydroxyethyl)-5-(dimethylamino)-[1,1′-biphenyl]-3-yl)methoxy)phenyl)acetic acid

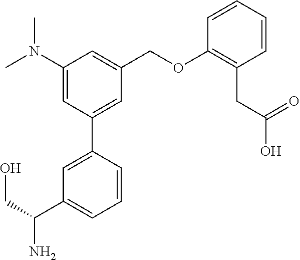 [SMILES: CN(C)c1cc(COc2ccccc2CC(=O)O)cc(-c2cccc([C@H](N)CO)c2)c1]

The title compound was synthesized as described in Example 128 using dimethylamine. ¹H NMR (400 MHz, METHANOL-d₄) δ ppm 7.69-7.79 (m, 2H) 7.53 (t, J=7.71 Hz, 1H) 7.42 (d, J=7.33 Hz, 1H) 7.19-7.28 (m, 3H) 7.10 (d, J=6.06 Hz, 2H) 7.02 (d, J=7.71 Hz, 1H) 6.88-6.95 (m, 1H) 5.19 (s, 2H) 4.43 (dd, J=8.27, 4.36 Hz, 1H) 3.82-4.05 (m, 2H) 3.70 (s, 2H) 3.11 (s, 6H). HRMS calcd. for C₂₅H₂₈N₁O₄ (M+H)⁺, 421.2083. found 421.2106.

### Example 133

### (S)-2-(2-((5-amino-3′-(1-amino-2-hydroxyethyl)-[1,1′-biphenyl]-3-yl)methoxy)phenyl)acetic acid

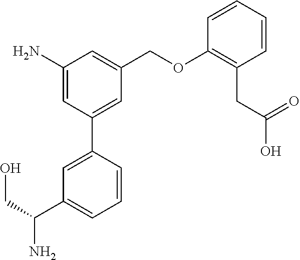 [SMILES: Nc1cc(COc2ccccc2CC(=O)O)cc(-c2cccc([C@H](N)CO)c2)c1]

The title compound was synthesized as described in Example 128 using cyclopropylamine. The cyclopropyl group was removed in the final deprotection step. ¹H NMR (400 MHz, METHANOL-d₄) δ ppm 7.78 (s, 1H) 7.68-7.74 (m, 1H) 7.51-7.59 (m, 2H) 7.45 (d, J=7.83 Hz, 1H) 7.29 (s, 1H) 7.20-7.26 (m, 2H) 7.18 (s, 1H) 7.00 (d, J=7.83 Hz, 1H) 6.90-6.96 (m, 1H) 5.20 (s, 2H) 4.44 (dd, J=8.08, 4.29 Hz, 1H) 3.81-4.08 (m, 2H) 3.71 (s, 2H). HRMS calcd. for C₂₃H₂₄N₂O₄ (M+H)⁺ 393.1814. found 393.1806.

### Example 134

### 2-(2-((3′-(Aminomethyl)-5-methoxy-[1,1′-biphenyl]-3-yl)methoxy)phenyl)acetic acid

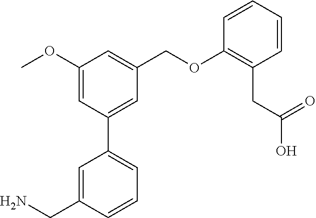 [SMILES: COc1cc(COc2ccccc2CC(=O)O)cc(-c2cccc(CN)c2)c1]

In a 2-5 mL microwave vial with stir bar was placed methyl 2-(2-((3-bromo-5-methoxybenzyl)oxy)phenyl)acetate (Intermediate 74) (0.125 g, 0.342 mmol) and (3-(aminomethyl)phenyl)boronic acid hydrochloride (CAS #146285-80-5) (0.096 g, 0.513 mmol) in DMF (3.1 mL) and water (0.342 mL). Then, 2M aq. K₃PO₄ (0.685 mL, 1.369 mmol) and PdCl₂(dppf).CH₂Cl₂ adduct (CAS #95464-05-4) (0.014 g, 0.017 mmol) were added. The vial was sealed and the reaction mixture heated in the microwave at 110° C. for 60 min. The reaction was diluted with water and EA and the EA layer was removed, dried over sodium sulfate, concentrated. The crude product was then dissolved in 1 mL of MeOH and then added 2M aq. NaOH (0.919 mL, 1.838 mmol) and heated at 55° C. for 1 hour. The reaction was concentrated, filtered and then purified by prep. HPLC (Method B) to obtain the title compound. ¹H NMR (400 MHz, DMSO-d₆ with 10 μL of TFA) δ ppm 8.19 (br. s.) 7.82 (s, 1H) 7.73 (d, J=7.82 Hz, 1H) 7.49-7.57 (m, 1H) 7.42-7.48 (m, 1H) 7.36 (s, 1H) 7.20-7.27 (m, 2H) 7.17 (t, J=1.83 Hz, 1H) 7.01-7.10 (m, 2H) 6.87-6.95 (m, 1H) 5.18 (s, 2H) 4.08-4.16 (m, 2H) 3.85 (s, 3H) 3.61 (s, 2H). HRMS calcd. for C₂₃H₂₃NO₄ (M+H)⁺ 378.1697. found 378.1705.

### Example 135

### (R)-2-(2-((3′-(Aminomethyl)-5-((tetrahydrofuran-2-yl)methoxy)-[1,1′-biphenyl]-3-yl)methoxy)phenyl)acetic acid

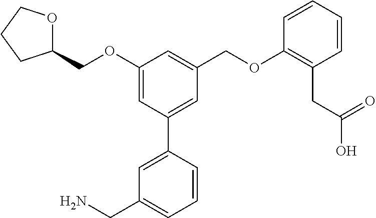 [SMILES: NCc1cccc(-c2cc(COc3ccccc3CC(=O)O)cc(OC[C@H]3CCCO3)c2)c1]

In a 2-5 mL microwave vial was placed (R)-methyl 2-(2-((3-bromo-5-((tetrahydrofuran-2-yl)methoxy)benzyl)oxy)phenyl)acetate (Intermediate 73-C) (0.1 g, 0.230 mmol) and (3-(aminomethyl)phenyl)boronic acid hydrochloride (CAS #146285-80-5) (0.065 g, 0.345 mmol) in DMF (2.1 mL) and water (0.230 mL). Then, 2M aq. K₃PO₄ (0.459 mL, 0.919 mmol) and PdCl₂(dppf).CH₂Cl₂ adduct (CAS #95464-05-4) (9.38 mg, 0.011 mmol) was added. The vial was sealed and the reaction was heated at 110° C. for 60 min. The mixture was diluted with EA and water, the EA layer was removed, dried and concentrated. The crude product was dissolved in 1 mL of MeOH and then added 2 M NaOH (1.149 mL, 2.297 mmol) and heated at 55° C. for 1 hour. The reaction was concentrated, filtered and then purified by preparative HPLC (Method B) to obtain the title compound. ¹H NMR (400 MHz, DMSO-d₆) δ ppm 8.18 (br. s.) 7.83 (s, 1H) 7.73 (d, J=7.83 Hz, 1H) 7.48-7.56 (m, 1H) 7.42-7.47 (m, 1H) 7.36 (s, 1H) 7.16-7.27 (m, 3H) 7.01-7.10 (m, 2H) 6.86-6.95 (m, 1H) 5.17 (s, 2H) 3.97-4.23 (m, 5H) 3.76-3.84 (m, 1H) 3.65-3.74 (m, 1H) 3.61 (s, 2H) 1.97-2.09 (m, 1H) 1.79-1.96 (m, 2H) 1.63-1.77 (m, 1H). HRMS calcd. for C₂₇H₂₉NO₅ (M+H)⁺ 448.2117. found 448.2124.

### Example 136

### 2-(2-((3′-(Aminomethyl)-5-(cyclopropylmethoxy)-[1,1′-biphenyl]-3-yl)methoxy)phenyl)acetic acid

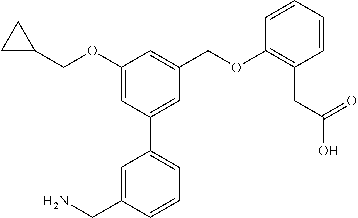 [SMILES: NCc1cccc(-c2cc(COc3ccccc3CC(=O)O)cc(OCC3CC3)c2)c1]

The title compound was synthesized in the same manner as Example 135 starting from methyl 2-(2-((3-bromo-5-(cyclopropylmethoxy)benzyl)oxy)phenyl)acetate (Intermediate 75-C) and (3-(aminomethyl)phenyl)boronic acid hydrochloride (CAS #146285-80-5). ¹H NMR (400 MHz, DMSO-d₆ with 10 μL TFA) δ ppm 7.86 (s, 1H) 7.73 (s, 1H) 7.59 (s, 1H) 7.53 (d, J=7.46 Hz, 1H) 7.33-7.40 (m, 1H) 7.25-7.32 (m, 1H) 7.00-7.20 (m, 4H) 6.88-6.97 (m, 1H) 6.73-6.84 (m, 1H) 5.20 (s, 2H) 5.09 (s, 2H) 4.23 (d, J=2.93 Hz, 1H) 3.92 (d, J=6.97 Hz, 2H) 3.76 (s, 1H) 1.10-1.35 (m, 1H) 0.48-0.68 (m, 2H) 0.24-0.43 (m, 2). HRMS calcd. for C₂₆H₂₇NO₄ (M+H)⁺ 418.2007. found 418.2018.

### Example 137

### 2-(2-((3′-(Aminomethyl)-2′,6-difluoro-[1,1′-biphenyl]-3-yl)methoxy)phenyl)acetic acid

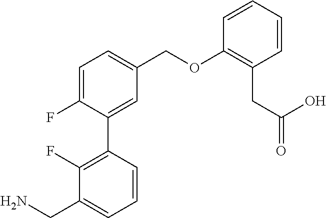 [SMILES: NCc1cccc(-c2cc(COc3ccccc3CC(=O)O)ccc2F)c1F]

The title compound was synthesized in the same manner as Example 135 from methyl 2-(2-((3-bromo-4-fluorobenzyl)oxy)phenyl)acetate (Intermediate 69) and (3-(aminomethyl)-2-fluorophenyl)boronic acid hydrochloride (CAS #1072946-44-1). ¹H NMR (400 MHz, DMSO-d₆) δ ppm 7.60-7.66 (m, 1H) 7.50-7.60 (m, 2H) 7.25-7.36 (m, 3H) 7.15-7.22 (m, 1H) 7.03-7.09 (m, 1H) 6.88-6.95 (m, 1H) 6.77-6.85 (m, 1H) 5.07-5.13 (m, 2H) 3.80 (s, 2H) 3.22-3.28 (m, 2H). HRMS calcd. for C₂₂H₁₉F₂NO₃ (M+H)⁺ 384.1407. found 384.1411.

### Example 138

The following compounds were prepared with a similar method as described in Example 135, using the appropriate boronic ester (Intermediate 76 or Example 101-B) and aryl halide (in table), and using either preparative HPLC Method A or Method B for purification.

| | | Aryl Halide/ | | |
|---|---|---|---|---|
| | Structure/Chemical Name | HPLC Method | ¹H NMR | HRMS |
| 138-A | 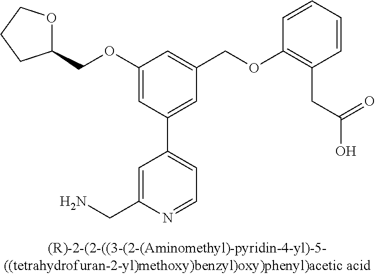 [SMILES: NCc1cc(-c2cc(COc3ccccc3CC(=O)O)cc(OC[C@H]3CCCO3)c2)ccn1] | (4-bromopyridin-2- yl)methanamine (CAS 865156-50-9)/ Method A. | (TFA salt, 400 MHz, DMSO-d₆) δ ppm 8.68 (d, J = 5.38 Hz, 1 H) 7.88 (s, 1 H) 7.79 (dd, J = 5.32, 1.77 Hz, 1 H) 7.49 (s, 1 H) 7.32 (t, J = 1.96 Hz, 1 H) 7.20-7.27 (m, 2 H) 7.17 (s, 1 H) 7.03 (d, J = 7.95 Hz, 1 H) 6.91 (td, J = 7.40, 0.98 Hz, 1 H) 5.19 (s, 2 H) 4.26 (br. s., 2 H) 4.19 (qd, J = 6.74, 3.97 Hz, 1 H) 3.99-4.11 (m, 2 H) 3.75-3.85 (m, 1 H) 3.70 (td, J = 7.70, 6.24 Hz, 1 H) 3.62 (s, 2 H) 1.97-2.08 (m, 1 H) 1.78- | calcd. for C₂₆H₂₈N₂O₅ (M + H)⁺ 449.2069, found 449.2076. |
| | | | 1.97 (m, 2 H) 1.64- | |
| | | | 1.76 (m. 1 H). | |
| 138-B | 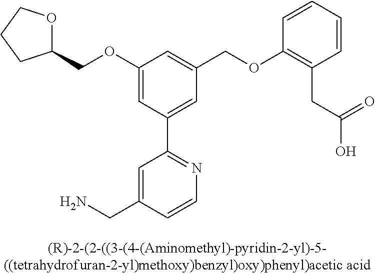 [SMILES: NCc1ccnc(-c2cc(COc3ccccc3CC(=O)O)cc(OC[C@H]3CCCO3)c2)c1] | (2-chloropyridin-4- yl)methanamine hydrochloride (CAS # 144900-57-2)./ Method A. | (TFA salt, 400 MHz, DMSO-d₆) δ ppm 8.71 (d, J = 5.13 Hz, 1 H) 8.34 (br. s., 2H) 8.09 (s, 1 H) 7.75 (s, 1 H) 7.55-7.62 (m, 1 H) 7.43 (dd, J = 5.07, 1.41 Hz, 1 H) 7.20-7.27 (m, 2 H) 7.16 (d, J = 0.73 Hz, 1 H) 7.01-7.07 (m, 1 H) 6.91 (td, J = 7.43, 0.92 Hz, 1 H) 5.19 (s, 2 H) 4.14- 4.23 (m, 3 H) 3.98-4.09 (m, 2 H) 3.77-3.84 (m, 1 H) 3.70 (td, J = 7.67, 6.17 Hz, 1 H) 3.61 (s, 2H) 1.98-2.08 (m, 1 H) 1.79- 1.97 (m, 2 H) 1.66- | calcd. for C₂₆H₂₈N₂O₅ (M + H)⁺ 449.2063, found 449.2076. |
| | | | 1.77 (m, 1 H). | |
| 138-C | 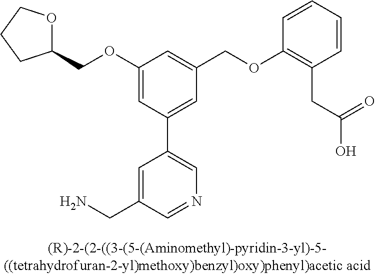 [SMILES: NCc1cncc(-c2cc(COc3ccccc3CC(=O)O)cc(OC[C@H]3CCCO3)c2)c1] | (5-bromopyridin-3- yl)methanamine (CAS # 135124-70-8)./ Method A. | (TFA salt, 400 MHz, DMSO-d₆) δ ppm 8.96 (d, J = 2.20 Hz, 1 H) 8.64 (d, J = 1.96 Hz, 1 H) 8.11- 8.29 (m, 3 H) 7.41 (s, 1 H) 7.20-7.26 (m, 3 H) 7.13 (s, 1 H) 7.03 (d, J = 7.95 Hz, 1 H) 6.91 (td, J = 7.43, 0.92 Hz, 1 H) 5.18 (s, 2 H) 4.14-4.23 (m, 3 H) 3.99-4.10 (m, 2 H) 3.76-3.84 (m, 1 H) 3.69 (td, J = 7.64, 6.24 Hz, 1 H) 3.61 (s, 2H) 1.97- 2.08 (m, 1 H) 1.79-1.97 (m, 2 H) 1.63- .75 (m, 1 H). | calcd. for C₂₆H₂₈N₂O₅ (M+H)⁺ 449.2061, found 449.2076. |
| 138-D | 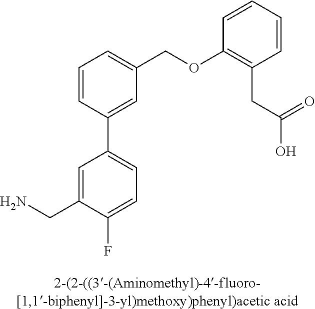 [SMILES: NCc1cc(-c2cccc(COc3ccccc3CC(=O)O)c2)ccc1F] | (5-bromo-2- fluorophenyl) methanamine (CAS # 190656-34-9)./ Method B. | ¹H NMR (400 MHz, DMSO-d₆ with 10 μL TFA) δ ppm 8.26 (br. s.) 7.90 (dd, J = 7.09, 2.32 Hz, 1 H) 7.72-7.82 (m, 2 H) 7.62 (d, J = 7.46 Hz, 1 H) 7.44 -7.55 (m, 2 H) 7.39 (dd, J = 9.72, 8.74 Hz, 1 H) 7.19-7.27 (m, 2 H) 7.06 (d, J = 7.82 Hz, 1 H) 6.87-6.96 (m, 1 H) 5.20 (s, 2 H) 4.17 (d, J = 5.75 Hz, 2 H) 3.60 (s, 2H). | calcd. for C₂₂H₂₀FNO₃ (M + H)⁺ 366.1508, found 366.1505. |
| 138-E | 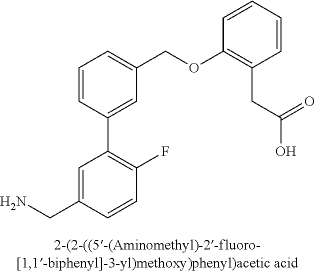 [SMILES: NCc1ccc(F)c(-c2cccc(COc3ccccc3CC(=O)O)c2)c1] | (3-bromo-4- fluorophenyl) methanamine hydrochloride (CAS # 388072-39-7)./ Method B. | ¹H NMR (400 MHz, DMSO-d₆ with 10 μL TFA) δ ppm 8.18 (br. s., 2 H) 7.62-7.75 (m, 2 H) 7.46-7.58 (m, 4 H) 7.40 (dd, J = 10.58, 8.62 Hz, 1 H) 7.19-7.28 (m, 2 H) 7.06 (d, J = 7.95 Hz, 1 H) 6.91 (t, J = 7.27 Hz, 1 H) 5.20 (s, 2H) 4.10 (d, J = 5.62 Hz, 2 H) 3.59 (s, 2 H). | calcd. for C₂₂H₂₀FNO₃ (M + H)⁺ 366.1494, found 366.1505. |
| 138-F | 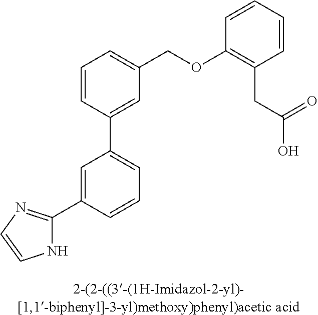 [SMILES: O=C(O)Cc1ccccc1OCc1cccc(-c2cccc(-c3ncc[nH]3)c2)c1] | Intermediate 77-B./ Method B. | (400 MHz, DMSO-d₆) δ ppm 8.80 (t, J = 1.59 Hz, 1 H) 8.28 (s, 1 H) 7.98 (dt, J = 7.86, 1.27 Hz, 1 H) 7.69 (d, J = 7.70 Hz, 1 H) 7.59-7.66 (m, 1 H) 7.43 (td, J = 7.67, 2.51 Hz, 2 H) 7.36-7.39 (m, 1 H) 7.01- 7.12 (m, 3 H) 6.84- 6.94 (m, 2 H) 6.78 (td, J = 7.34, 0.98 Hz, 1 H) 5.18 (s, 2 H) 3.37 (s, 2 H). | calcd. for C₂₄H₂₀N₂O₃ (M + H)⁺ 385.1546, found 385.1552. |
| 138-G | 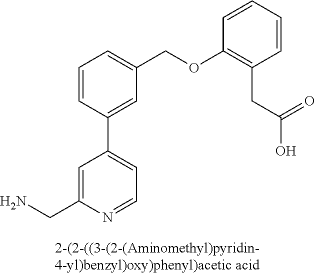 [SMILES: NCc1cc(-c2cccc(COc3ccccc3CC(=O)O)c2)ccn1] | (4-bromopyridin-2- yl)methanamine/ Method A. | (TFA salt, 400 MHz, DMSO-d₆) δ ppm 8.69 (d, J = 5.26 Hz, 1 H) 8.31 (br. s., 2H) 7.92 (s, 1 H) 7.87 (s, 1 H) 7.72-7.81 (m, 2 H) 7.51-7.63 (m, 2 H) 7.18-7.29 (m, 2 H) 7.06 (d, J = 7.83 Hz, 1 H) 6.92 (td, J = 7.40, 0.98 Hz, 1 H) 5.23 (s, 2 H) 4.28 (br. s., 2 H) 3.61 (s, 2H). | calcd. for C₂₁H₂₀N₂O₃ (M + H)⁺ 349.1547, found 349.1552. |
| 138-H | 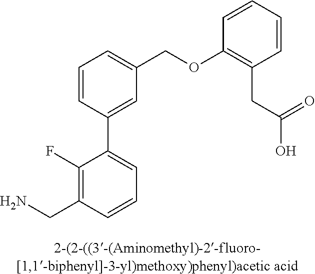 [SMILES: NCc1cccc(-c2cccc(COc3ccccc3CC(=O)O)c2)c1F] | (3-bromo-2- fluorophenyl) methanamine (CAS # 261723-28-8). / Method B. | ¹H NMR @ 120° C. (600 MHz, DMSO-d₆) δ ppm 7.62 (s, 1 H) 7.52 (d, J = 4.31 Hz, 1 H) 7.40- 7.46 (m, 3 H) 7.36 (td, J = 7.57, 1.65 Hz, 1 H) 7.29 (dd, J = 7.47, 1.42 Hz, 1 H) 7.16-7.23 (m, 1 H) 6.95-7.02 (m, 1 H) 6.86-6.92 (m, 1 H) 6.79 (td, J = 7.36, 1.05 Hz, 1 H) 5.11 (s, 2 H) 3.85 (s, 2 H) 3.24 (s, 2 H). | calcd. for C₂₂H₂₀FNO₃ (M + H)⁺ 366.1502, found 366.1505. |
| 138-I | 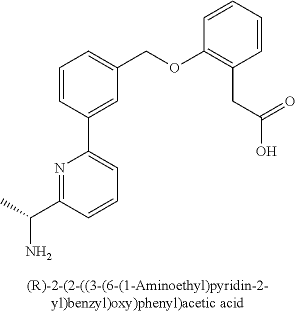 [SMILES: C[C@@H](N)c1cccc(-c2cccc(COc3ccccc3CC(=O)O)c2)n1] | (R)-1-(6- bromopyridin-2- yl)ethanamine hydrochloride (CAS # 1415303-41-1)./ Method A. | (TFA salt, 400 MHz, DMSO-d₆) δ ppm 8.34 (br. s., 2H) 8.28 (s, 1 H) 8.19-8.24 (m, 1 H) 7.95- 8.04 (m, 2 H) 7.51- 7.58 (m, 2 H) 7.48 (dd, J = 6.85, 1.71 Hz, 1 H) 7.19-7.27 (m, 2 H) 7.07 (d, J = 7.82 Hz, 1 H) 6.91 (td, J = 7.40, 0.86 Hz, 1 H) 5.22 (s, 2 H) 4.57-4.68 (m, 1 H) 3.61 (s, 2H) 1.56 (d, J = 6.85 Hz, 3 H). | calcd. for C₂₂H₂₂N₂O₃ (M + H)⁺ 363.1703, found 363.1709. |
| 138-J | 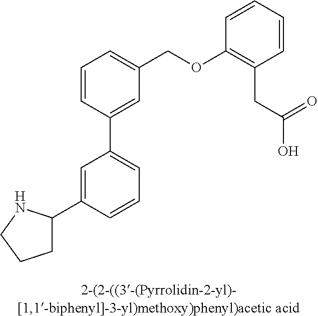 [SMILES: O=C(O)Cc1ccccc1OCc1cccc(-c2cccc(C3CCCN3)c2)c1] | 2-(3- bromophenyl) pyrrolidine hydrochloride (CAS # 1171898-22-8)./ Method B. | (400 MHz, DMSO-d₆) δ ppm 7.81 (d, J = 7.58 Hz, 2 H) 7.76 (d, J = 7.82 Hz, 1 H) 7.67 (d, J = 7.21 Hz, 1 H) 7.42-7.61 (m, 4 H) 7.18-7.30 (m, 2 H) 7.07 (d, J = 7.95 Hz, 1 H) 6.92 (t, J = 7.09 Hz, 1 H) 5.22 (s, 2 H) 4.65 (d, J = 6.36 Hz, 1 H) 3.61 (s, 2H) 3.23-3.48 (m, 2 H) 2.40- 2.48 (m, 1 H) 1.96- 2.22 (m, 3H). | calcd. for C₂₅H₂₅NO₃ (M + H)⁺ 388.1906, found 388.1913. |
| 138-K | 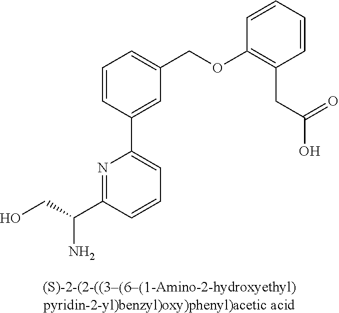 [SMILES: N[C@H](CO)c1cccc(-c2cccc(COc3ccccc3CC(=O)O)c2)n1] | (S)-2-amino-2-(6- bromopyridin-2- yl)ethanol hydrochloride (CAS# 1213580-51-8)./ Method A. | (TFA salt, 400 MHz, DMSO-d₆) δ ppm 8.39 (br. s., 2H) 8.28 (s, 1 H) 8.22 (dt, J = 6.24, 2.26 Hz, 1 H) 7.90-8.05 (m, 2 H) 7.52-7.57 (m, 2 H) 7.42- 7.52 (m, 1 H) 7.17-7.28 (m, 2 H) 7.06 (d, J = 7.95 Hz, 1 H) 6.83-6.96 (m, 1 H) 5.49 (br. s., 1 H) 5.22 (s, 2 H) 4.53 (d, J = 4.16 Hz, 1 H) 3.86-3.94 (m, 1 H) 3.82 (d, J = 7.09 Hz, 1 H) 3.60 (s, 2 H). | calcd. for C₂₂H₂₂N₂O₄ (M + H)⁺ 379.1688, found 379.1658. |
| 138-L | 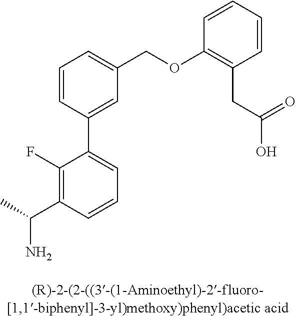 [SMILES: C[C@@H](N)c1cccc(-c2cccc(COc3ccccc3CC(=O)O)c2)c1F] | (R)-1-(3-chloro-2- fluorophenyl) ethanamine hydrochloride (CAS# 1253792-97-0)./ Method B. S-Phos palladacycle was used instead of PdCl₂(dppf)•CH₂Cl₂ adduct in the coupling step. | (400 MHz, DMSO-d₆) δ ppm 8.36 (br. s., 2 H) 7.64 (s, 1 H) 7.55-7.62 (m, 2 H) 7.47-7.55 (m, 3 H) 7.37-7.44 (m, 1 H) 7.19-7.27 (m, 2 H) 7.06 (d, J = 7.82 Hz, 1 H) 6.91 (td, J = 7.40, 0.86 Hz, 1 H) 5.20 (s, 2 H) 4.64-4.78 (m, 1 H) 3.58 (s, 2 H) 1.57 (d, J = 6.85 Hz, 3 H). | calcd. for C₂₃H₂₂FNO₃ (M + H)⁺ 380.1672, found 380.1662. |
| 138-M | 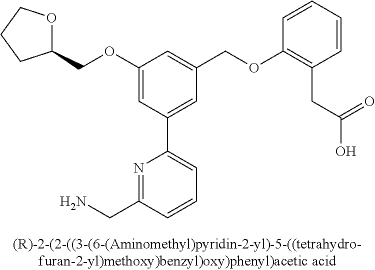 [SMILES: NCc1cccc(-c2cc(COc3ccccc3CC(=O)O)cc(OC[C@H]3CCCO3)c2)n1] | (6-bromopyridin-2- yl)methanamine hydrochloride (CAS # 914947-27-5)./ Method A. | (TFA salt, 400 MHz, DMSO-d₆) δ ppm 8.34 (br. s., 2 H) 7.91-8.03 (m, 2 H) 7.82 (s, 1 H) 7.75-7.80 (m, 1 H) 7.43 (d, J = 7.21 Hz, 1 H) 7.20- 7.27 (m, 2 H) 7.14 (d, J = 0.86 Hz, 1 H) 6.99- 7.07 (m, 1 H) 6.91 (td, J = 7.40, 0.86 Hz, 1 H) 5.19 (s, 2 H) 4.29 (q, J = 5.67 Hz, 2 H) 4.20 (qd, J = 6.72, 4.03 Hz, 1 H) 4.00-4.12 (m, 2 H) 3.77- 3.85 (m, 1 H) 3.70 (td, J = 7.67, 6.30 Hz, 1 H) 3.62 (s, 2 H) 1.98-2.08 | calcd. for C₂₆H₂₈N₂O₅ (M + H)⁺ 449.2074, found 449.2076. |
| | | | (m, 1 H) 1.79-1.97 (m, | |
| | | | 2 H) 1.64-1.75 (m, 1 H). | |
| 138-N | 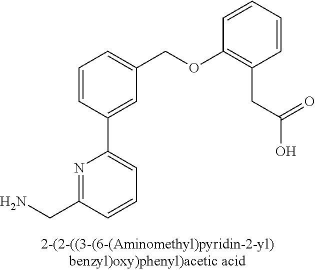 [SMILES: NCc1cccc(-c2cccc(COc3ccccc3CC(=O)O)c2)n1] | (6-bromopyridin-2- yl)methanamine hydrochloride/ Method A. | (TFA salt, 400 MHz, DMSO-d₆) δ ppm 8.26- 8.41 (m, 3 H) 8.21 (dt, J = 6.42, 2.23 Hz, 1 H) 7.90-8.05 (m, 2 H) 7.49- 7.59 (m, 2 H) 7.38- 7.47 (m, 1 H) 7.18-7.28 (m, 2 H) 7.06 (d, J = 7.83 Hz, 1 H) 6.91 (td, J = 7.40, 0.86 Hz, 1 H) 5.22 (s, 2 H) 4.09-4.40 (m, 2 H) 3.61 (s, 2 H). | calcd. for C₂₁H₂₀N₂O₃ (M + H)⁺ 349.1553, found 349.1552. |

### Example 139

### 2-(2-((3′-(Aminomethyl)-5-((cyclopropylmethyl)amino)-[1,1′-biphenyl]-3-yl)methoxy)phenyl)acetic acid

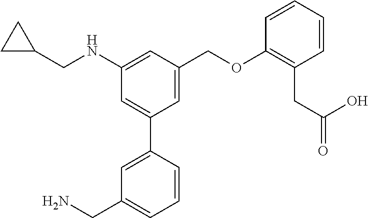 [SMILES: NCc1cccc(-c2cc(COc3ccccc3CC(=O)O)cc(NCC3CC3)c2)c1]

A suspension of tert-butyl 2-(2-((3′-(((tert-butoxycarbonyl)amino)methyl)-5-chloro-[1,1′-biphenyl]-3-yl)methoxy)phenyl)acetate (Example 102-A) (110 mg, 0.204 mmol), cyclopropylmethanamine (21.81 mg, 0.307 mmol), BrettPhos palladacycle (8.17 mg, 10.22 μmol) and Cs₂CO₃ (333 mg, 1.022 mmol) in CH₃CN (2 ml) was heated at 140° C. for 60 min in a microwave. The product was passed through a pad of silica eluting with 1:1 heptane/EtOAc. The resulting residue (103 mg, 0.180 mmol) was taken up in DCM (0.899 mL) and TFA (0.139 mL, 1.798 mmol) was added and the solution was stirred at room temperature for 22 hours. The reaction was concentrated and purified via preparative HPLC (Method B) to obtain the title compound. ¹H NMR (400 MHz, DMSO-d₆) δ ppm 8.14 (s, 1H) 7.56 (d, J=7.95 Hz, 1H) 7.37 (t, J=7.64 Hz, 1H) 7.19-7.27 (m, 2H) 7.08 (qd, J=7.36, 1.65 Hz, 2H) 6.89 (d, J=7.70 Hz, 1H) 6.76-6.84 (m, 2H) 6.58 (s, 1H) 5.74 (br. s., 1H) 5.08 (s, 2H) 3.94 (s, 2H) 3.40 (s, 2H) 2.94-3.01 (m, 2H) 1.01-1.15 (m, 1H) 0.45-0.53 (m, 2H) 0.20-0.28 (m, 2H). HRMS calcd. for C₂₆H₂₈N₂O₃ (M+H)⁺ 417.2175. found 417.2178.

### Example 140

### (S)-2-(2-((3′-(Aminomethyl)-5-(((tetrahydrofuran-2-yl)methyl)amino)-[1,1′-biphenyl]-3-yl)methoxy)phenyl)acetic acid

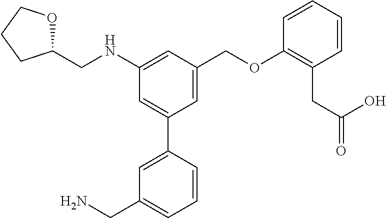 [SMILES: NCc1cccc(-c2cc(COc3ccccc3CC(=O)O)cc(NC[C@@H]3CCCO3)c2)c1]

The title compound was synthesized in the same manner as Example 139 using (S)-(tetrahydrofuran-2-yl)methanamine (CAS #7175-81-7) in place of cyclopropylmethanamine. ¹H NMR (400 MHz, DMSO-d₆) δ ppm 8.14 (s, 1H) 7.56 (d, J=7.82 Hz, 1H) 7.37 (t, J=7.64 Hz, 1H) 7.19-7.28 (m, 2H) 7.02-7.13 (m, 2H) 6.83-6.92 (m, 2H) 6.79 (td, J=7.34, 0.86 Hz, 1H) 6.61 (s, 1H) 5.66 (t, J=5.81 Hz, 1H) 5.08 (s, 2H) 3.99-4.07 (m, 1H) 3.94 (s, 2H) 3.75-3.84 (m, 1H) 3.61-3.70 (m, 1H) 3.40 (s, 2H) 3.07-3.23 (m, 2H) 1.94-2.04 (m, 1H) 1.77-1.92 (m, 2H) 1.56-1.68 (m, 1H). HRMS calcd. for C₂₇H₃₀N₂O₄ (M+H)⁺ 447.2273. found 447.2284.

### Example 141

### Example 141-A

### (S)-tert-Butyl 2-(2-((3′-(1-amino-2-hydroxyethyl)-2′-chloro-[1,1′-biphenyl]-3-yl)methoxy)phenyl)acetate

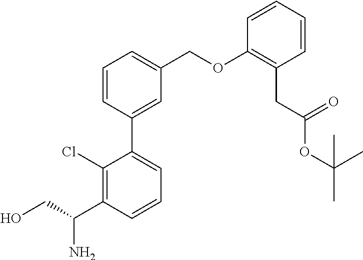 [SMILES: CC(C)(C)OC(=O)Cc1ccccc1OCc1cccc(-c2cccc([C@H](N)CO)c2Cl)c1]

In a 2-5 mL microwave vial with stir bar was placed tert-butyl 2-(2-((3-(4,4,5,5-tetramethyl-1,3,2-dioxaborolan-2-yl)benzyl)oxy)phenyl)acetate (Intermediate 78-B) (0.14 g, 0.330 mmol) and (S)-2-amino-2-(3-bromo-2-chlorophenyl)ethanol hydrochloride (CAS #1388090-97-8) (0.114 g, 0.396 mmol) in DMF (1.9 mL) and water (0.2 mL). Then, 2M aq. K₃PO₄ (0.660 mL, 1.320 mmol) and PdCl₂(dppf).CH₂Cl₂ adduct (CAS #95464-05-4) (0.013 g, 0.016 mmol) was added. The vial was sealed and the reaction was heated at 110° C. for 60 min. The reaction was diluted with EA and water and the water layer was removed, dried and concentrated and purified via FCC (0-10% MeOH (containing 10% ammonium hydroxide):DCM to obtain the title compound. MS (ESI+) m/z 468.1 (M+H).

### Example 141-B

### (S)-2-(2-((3′-(1-Amino-2-hydroxyethyl)-2′-chloro-[1,1′-biphenyl]-3-yl)methoxy)phenyl)acetic acid

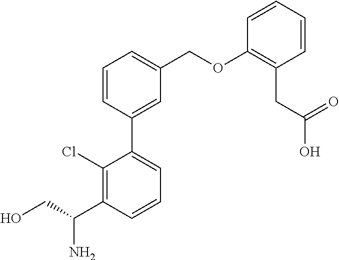 [SMILES: N[C@H](CO)c1cccc(-c2cccc(COc3ccccc3CC(=O)O)c2)c1Cl]

(S)-tert-butyl 2-(2-((3′-(1-amino-2-hydroxyethyl)-2′-chloro-[1,1′-biphenyl]-3-yl)methoxy)phenyl)acetate (0.14 g, 0.299 mmol) was dissolved in DCM (1.5 mL) and treated with 1 mL of TFA at 0° C. and then allowed to warm to room temperature over 1 hour. The reaction was concentrated and purified via preparative HPLC (Method B) to provide the title compound. ¹H NMR (400 MHz, DMSO-d₆) δ ppm 7.68 (dd, J=7.70, 1.59 Hz, 1H) 7.43-7.50 (m, 3H) 7.39 (t, J=7.64 Hz, 1H) 7.30-7.36 (m, 1H) 7.17-7.28 (m, 3H) 7.06 (d, J=7.82 Hz, 1H) 6.90 (td, J=7.40, 0.86 Hz, 1H) 5.17 (s, 2H) 4.40 (dd, J=7.64, 3.85 Hz, 1H) 3.60 (dd, J=10.45, 3.97 Hz, 1H) 3.56 (s, 2H) 3.25 (dd, J=10.45, 7.76 Hz, 1H). HRMS calcd. for C₂₃H₂₂ClNO₄ (M+H)⁺ 412.1314. found 412.1316.

### Example 142

The following compounds were prepared with similar methods as described in Example 141-A, using the boronic ester from Intermediate 78-B and the appropriate aryl halide (in table), and using either deprotection methods from either Example 141-B or Example 114-B.

| | | Aryl Halide/ | | |
|---|---|---|---|---|
| | Structure/Chemical Name | Deprotection Method | ¹H NMR | HRMS |
| 142-A | 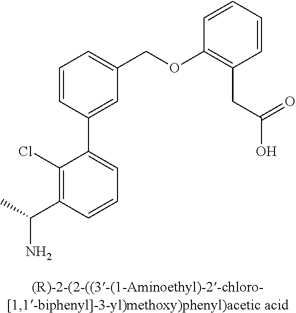 [SMILES: C[C@@H](N)c1cccc(-c2cccc(COc3ccccc3CC(=O)O)c2)c1Cl] | (R)-1-(3-bromo-2- chlorophenyl) ethanamine hydrochloride (CAS # 1389354-65-7)./ Example 141-B. | (400 MHz, DMSO-d₆) δ ppm 7.72 (dd, J = 7.83, 1.59 Hz, 1 H) 7.45- 7.51 (m, 3 H) 7.37- 7.44 (m, 1 H) 7.33 (dt, J = 6.82, 1.79 Hz, 1 H) 7.15-7.28 (m, 3 H) 7.05 (d, J = 7.83 Hz, 1 H) 6.89 (td, J = 7.40, 0.86 Hz, 1 H) 5.17 (s, 2H) 4.46 (d, J = 6.48 Hz, 1 H) 3.55 (s, 2 H) 1.13-1.35 (m, 3 H). | calcd. for C₂₃H₂₂ClNO₃ (M + H)⁺ 396.1382, found 396.1366. |
| 142-B | 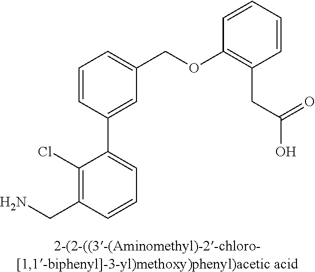 [SMILES: NCc1cccc(-c2cccc(COc3ccccc3CC(=O)O)c2)c1Cl] | (3-bromo-2- chlorophenyl) methanamine (Intermediate 39-B)./ Example 141-B. | (400 MHz, DMSO-d₆) δ ppm 7.59 (dd, J = 7.58, 1.47 Hz, 1 H) 7.43- 7.51 (m, 3 H) 7.40 (t, J = 7.64 Hz, 1 H) 7.30- 7.36 (m, 1 H) 7.26 (dd, J = 7.52, 1.65 Hz, 1 H) 7.20 (d, J = 7.46 Hz, 2 H) 7.05 (d, J = 7.95 Hz, 1 H) 6.86-6.92 (m, 1 H) 5.17 (s, 2 H) 3.85 (s, 2 H) 3.54 (s, 2 H). | calcd. for C₂₂H₂₀ClNO₃ (M + H)⁺ 382.1218, found 382.1210. |
| 142-C | 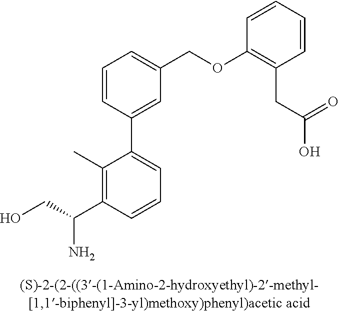 [SMILES: Cc1c(-c2cccc(COc3ccccc3CC(=O)O)c2)cccc1[C@H](N)CO] | (S)-2-amino-2- (3-bromo-2- methylphenyl) ethanol hydrochloride (CAS # 1212905-62-8)./ Example 141-B. | (400 MHz, DMSO-d₆) δ ppm 7.36-7.47 (m, 3 H) 7.18-7.28 (m, 5 H) 7.14 (d, J = 7.82 Hz, 1 H) 7.04 (d, J = 7.82 Hz, 1 H) 6.89 (td, J = 7.40, 0.98 Hz, 1 H) 5.17 (s, 2H) 3.89 (dd, J = 7.83, 4.65 Hz, 1 H) 3.55 (s, 2H) 3.50 (dd, J = 10.45, 4.71 Hz, 1 H) 3.35 (dd, J = 10.39, 7.95 Hz, 1 H) 2.20 (s, 3 H). | calcd. for C₂₄H₂₅NO₄ (M + H)⁺ 392.1877, found 392.1862. |
| 142-D | 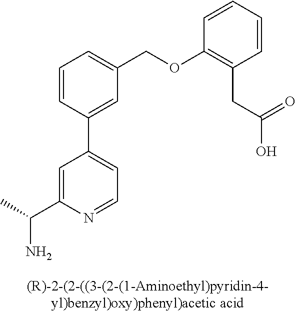 [SMILES: C[C@@H](N)c1cc(-c2cccc(COc3ccccc3CC(=O)O)c2)ccn1] | (R)-1-(4- bromopyridin-2- yl)ethanamine hydrochloride (CAS # 1213368-93-4)./ Example 141-B. | (400 MHz, DMSO-d₆) δ ppm 8.57 (d, J = 5.26 Hz, 1 H) 8.35 (s, 1 H) 8.18 (s, 1 H) 7.80 (dt, J = 6.94, 1.79 Hz, 1 H) 7.72 (dd, J = 5.26, 1.59 Hz, 1 H) 7.45-7.56 (m, 2 H) 7.07-7.18 (m, 2 H) 6.95 (d, J = 7.95 Hz, 1 H) 6.74-6.87 (m, 1 H) 5.22 (s, 2 H) 4.30 (d, J = 6.85 Hz, 1 H ) 3.31- 3.51 (m, 2 H) 1.53 (d, J = 6.85 Hz, 3H). | calcd. for C₂₂H₂₂N₂O₃ (M + H)⁺ 363.1731, found 363.1709. |
| 142-E | 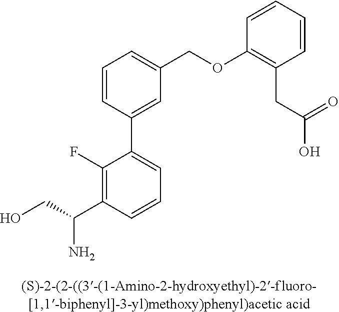 [SMILES: N[C@H](CO)c1cccc(-c2cccc(COc3ccccc3CC(=O)O)c2)c1F] | (S)-2-amino-2- (3-chloro-2- fluorophenyl) ethanol hydrochloride (CAS # 1391506-22-1)./ Example 141-B. S-Phos palladacycle was used instead of PdCl₂(dppf)•CH₂Cl₂ adduct in the coupling step. | (400 MHz, DMSO-d₆) δ ppm 8.47 (d, J = 4.28 Hz, 2 H) 7.55-7.65 (m, 3 H) 7.47-7.54 (m, 3H) 7.35-7.44 (m, 1 H) 7.17-7.28 (m, 2 H) 7.06 (d, J = 7.95 Hz, 1 H) 6.87-6.95 (m, 1 H) 5.20 (s, 2 H) 4.59 (d, J = 5.87 Hz, 1 H) 3.68- 3.91 (m, 2 H) 3.58 (s, 2 H). | calcd. for C₂₃H₂₂FNO₄ (M + H)⁺ 396.1634, found 396.1611. |
| 142-F | 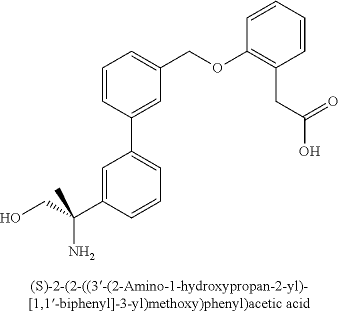 [SMILES: C[C@@](N)(CO)c1cccc(-c2cccc(COc3ccccc3CC(=O)O)c2)c1] | (S)-2-amino-2-(3- bromophenyl) propan-1-ol hydrochloride (CAS # 1391575-22-6)./ Example 114-B. | (400 MHz, DMSO-d₆) δ ppm 8.20 (s, 1 H) 7.99 (s, 1 H) 7.54-7.65 (m, 2 H) 7.31-7.40 (m, 3 H) 7.24-7.31 (m, 1 H) 6.96-7.07 (m, 2H) 6.86 (d, J = 7.95 Hz, 1 H) 6.73 (td, J = 7.34, 0.98 Hz, 1 H) 5.14 (s, 2H) 3.55-3.62 (m, 1 H) 3.36-3.51 (m, 2 H) 3.23-3.32 (m, 1 H) 1.48 (s, 3 H). | calcd. for C₂₄H₂₅NO₄ (M + H)⁺ 392.1856, found 392.1862. |
| 142-G | 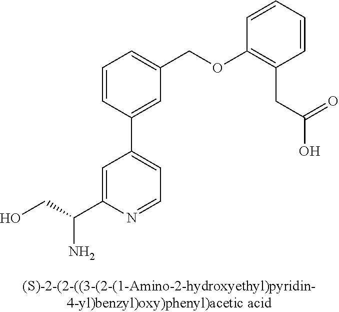 [SMILES: N[C@H](CO)c1cc(-c2cccc(COc3ccccc3CC(=O)O)c2)ccn1] | (S)-2-amino-2- (4-bromopyridin- 2-yl)ethanol hydrochloride (CAS# 1213131-57-7)./ Example 114-B. | (400 MHz, DMSO-d₆) δ ppm 8.49-8.62 (m, 1 H) 8.22 (s, 1 H) 8.08 (s, 1 H) 7.75-7.85 (m, 1 H) 7.70 (dd, J = 5.20, 1.65 Hz, 1 H) 7.52 (d, J = 5.01 Hz, 2 H) 7.07-7.17 (m, 2 H) 6.97 (d, J = 7.95 Hz, 1 H) 6.77-6.88 (m, 1 H) 5.23 (s, 2H) 4.19 (dd, J = 6.85, 3.91 Hz, 1 H) 4.05 (s, 1 H) 3.85 (dd, J = 11.43, 3.97 Hz, 1 H) 3.71 (dd, J = 11.37, 6.97 Hz, 1 H) 3.46 (q, J = 15.53 Hz, 2H). | calcd. for C₂₂H₂₂N₂O₄ (M + H)⁺ 379.1664, found 379.1658. |
| 142-H | 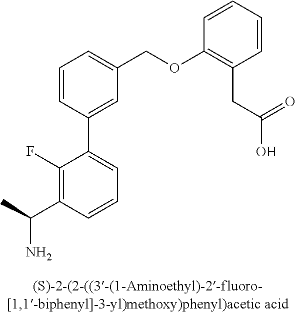 [SMILES: C[C@H](N)c1cccc(-c2cccc(COc3ccccc3CC(=O)O)c2)c1F] | (S)-1-(3-chloro-2- fluorophenyl) ethanamine hydrochloride (CAS # 1313593-59-7)./ Example 114-B. S-Phos palladacycle was used instead of PdCl(dppf)•CH₂Cl₂ adduct in the coupling step. | (400 MHz, DMSO-d₆) δ ppm 7.67 (s, 1 H) 7.52- 7.60 (m, 1 H) 7.47 (s, 3 H) 7.38 (td, J = 7.52, 1.59 Hz, 1 H) 7.15-7.30 (m, 3 H) 7.03 (d, J = 7.82 Hz, 1 H) 6.88 (td, J = 7.40, 0.86 Hz, 1 H) 5.19 (s, 2 H) 4.36 (d, J = 6.48 Hz, 1 H) 3.53 (s, 2 H) 1.34 (d, J = 6.60 Hz, 3 H). | calcd. for C₂₃H₂₂FNO₃ (M + H)⁺ 380.1653, found 380.1662. |
| 142-I | 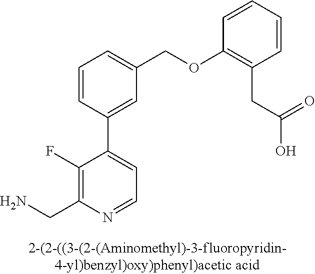 [SMILES: NCc1nccc(-c2cccc(COc3ccccc3CC(=O)O)c2)c1F] | (4-chloro-3- fluoropyridin-2- yl)methanamine hydrochloride (CAS # 1260903-03-4)./ Example 114-B. S-Phos palladacycle was used instead of PdCl₂(dppf)•CH₂Cl₂ adduct in the coupling step. | (TFA salt, 400 MHz, DMSO-d₆) δ ppm 8.56 (d, J = 5.01 Hz, 1 H) 8.38 (br. s., 2 H) 7.77 (s, 1 H) 7.71 (t, J = 5.56 Hz, 1 H) 7.55-7.66 (m, 3 H) 7.19-7.27 (m, 2 H) 7.06 (d, J = 7.82 Hz, 1 H) 6.92 (td, J = 7.40, 0.86 Hz, 1 H) 5.22 (s, 2H) 4.36 (d, J = 4.89 Hz, 2 H) 3.59 (s, 2 H). | calcd. for C₂₁H₁₉FN₂O₃ (M + H)⁺ 367.1439, found 367.1458. |
| 142-J | 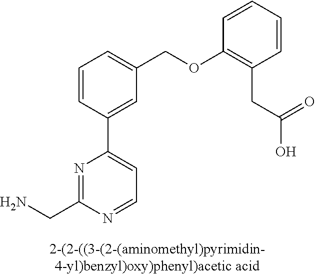 [SMILES: NCc1nccc(-c2cccc(COc3ccccc3CC(=O)O)c2)n1] | tert-butyl ((4- chloropyrimidin-2- yl)methyl) carbamate (Intermediate 38)./ Example 141-B. | (400 MHz, DMSO-d₆) δ ppm 8.83 (d, J = 5.26 Hz, 1 H) 8.77 (s. 1 H) 8.11 (s, 1 H) 8.00 (d, J = 5.38 Hz, 1 H) 7.56 (dt, J = 14.89, 7.53 Hz, 2H) 7.05-7.16 (m, 2 H) 6.92 (s, 1 H) 6.83 (td, J = 7.37, 0.79 Hz, 1 H) 5.22 (s, 2 H) 4.13 (s, 2 H) 3.40 (s, 2H). | calcd. for C₂₀H₁₉N₃O₃ (M + H)⁺ 350.1524, found 350.1505. |

### Example 143

### Example 143-A

### tert-Butyl 2-(2-((3′-cyano-2′-methyl-[1,1′-biphenyl]-3-yl)methoxy)phenyl)acetate

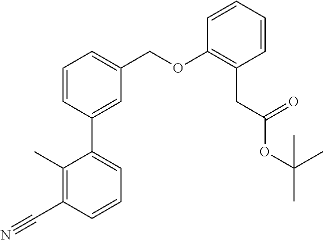 [SMILES: Cc1c(C#N)cccc1-c1cccc(COc2ccccc2CC(=O)OC(C)(C)C)c1]

The title compound was synthesized in the same manner as Example 141-A (except using S-Phos palladacycle instead of PdCl₂(dppf).CH₂Cl₂ adduct as a catalyst) from tert-butyl 2-(2-((3-(4,4,5,5-tetramethyl-1,3,2-dioxaborolan-2-yl)benzyl)oxy)phenyl)acetate (Intermediate 78-B) and 3-chloro-2-methylbenzonitrile (CAS #54454-12-5). MS (ESI+) m/z 358.0 (M−tBu+H).

### Example 143-B

### tert-Butyl 2-(2-((3′-(aminomethyl)-2′-methyl-[1,1′-biphenyl]-3-yl)methoxy)phenyl)acetate

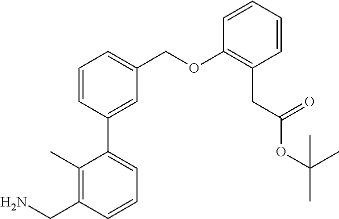 [SMILES: Cc1c(CN)cccc1-c1cccc(COc2ccccc2CC(=O)OC(C)(C)C)c1]

To tert-butyl 2-(2-((3′-cyano-2′-methyl-[1,1′-biphenyl]-3-yl)methoxy)phenyl)acetate (0.144 g, 0.348 mmol) in EtOH (6.9 ml) was added NiCl₂ (0.090 g, 0.696 mmol) and the mixture was cooled in an ice bath. Sodium borohydride (0.053 g, 1.393 mmol) was added and the ice bath was removed. Further aliquots of NiCl₂ (0.090 g, 0.696 mmol) and sodium borohydride (0.053 g, 1.393 mmol) and 1 mL of MeOH were added after 10 minutes. After 10 minutes the reaction was filtered through Celite® and washed with methanol (250 mL) and concentrated and absorbed onto silica and purified via FCC (0-10% MeOH containing 10% Ammonium Hydroxide:DCM) to obtain the title compound. MS (ESI+) m/z 418.2 (M+H).

### Example 143-C

### 2-(2-((3′-(Aminomethyl)-2′-methyl-[1,1′-biphenyl]-3-yl)methoxy)phenyl)acetic acid

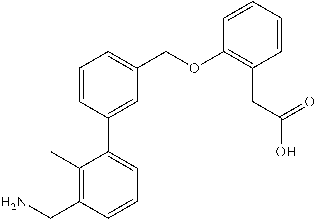 [SMILES: Cc1c(CN)cccc1-c1cccc(COc2ccccc2CC(=O)O)c1]

The title compound was synthesized in the same manner as Example 141-B from tert-butyl 2-(2-((3′-(aminomethyl)-2′-methyl-[1,1′-biphenyl]-3-yl)methoxy)phenyl)acetate. ¹H NMR (400 MHz, DMSO-d₆) δ ppm 7.39-7.48 (m, 3H) 7.35 (d, J=7.46 Hz, 1H) 7.08-7.28 (m, 5H) 6.98 (d, J=7.83 Hz, 1H) 6.86 (td, J=7.40, 0.86 Hz, 1H) 5.19 (s, 2H) 3.83 (s, 2H) 3.44-3.51 (m, 2H) 2.13 (s, 3H). HRMS calcd. for C₂₃H₂₃NO₃ (M+H)⁺ 362.1754. found 362.1756.

### Example 144

### Example 144-A

### tert-Butyl 2-(2-((3′-cyano-2′-hydroxy-[1,1′-biphenyl]-3-yl)methoxy)phenyl)acetate

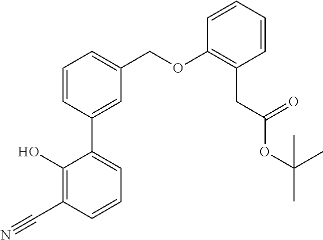 [SMILES: CC(C)(C)OC(=O)Cc1ccccc1OCc1cccc(-c2cccc(C#N)c2O)c1]

The title compound was synthesized in the same manner as Example 141-A from tert-butyl 2-(2-((3-(4,4,5,5-tetramethyl-1,3,2-dioxaborolan-2-yl)benzyl)oxy)phenyl)acetate (Intermediate 78-B) and 3-bromo-2-hydroxybenzonitrile (CAS #13073-28-4). MS (ESI+) m/z 416.1 (M+H).

### Example 144-B

### tert-Butyl 2-(2-((3′-(aminomethyl)-2′-hydroxy-[1,1′-biphenyl]-3-yl)methoxy)phenyl)acetate

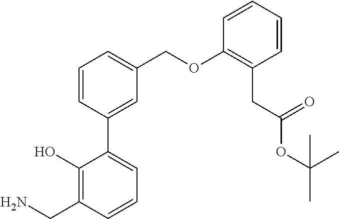 [SMILES: CC(C)(C)OC(=O)Cc1ccccc1OCc1cccc(-c2cccc(CN)c2O)c1]

The title compound was synthesized in the same manner as Example 143-B from tert-butyl 2-(2-((3′-cyano-2′-hydroxy-[1,1′-biphenyl]-3-yl)methoxy)phenyl)acetate. MS (ESI+) m/z 419.9 (M+H).

### Example 144-C

### 2-(2-((3′-(Aminomethyl)-2′-hydroxy-[1,1′-biphenyl]-3-yl)methoxy)phenyl)acetic acid

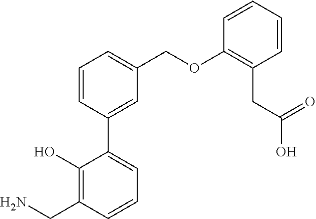 [SMILES: NCc1cccc(-c2cccc(COc3ccccc3CC(=O)O)c2)c1O]

The title compound was synthesized in the same manner as Example 114-B from tert-butyl 2-(2-((3′-(aminomethyl)-2′-hydroxy-[1,1′-biphenyl]-3-yl)methoxy)phenyl)acetate. ¹H NMR (400 MHz, DMSO-d₆) δ ppm 7.68 (s, 1H) 7.30-7.47 (m, 3H) 7.19 (t, J=8.80 Hz, 3H) 7.00-7.11 (m, 2H) 6.79-6.91 (m, 2H) 5.16 (s, 2H) 4.00 (s, 2H) 3.57 (s, 1H) 3.52 (s, 2H). HRMS calcd. for C₂₂H₂₁NO₄ (M+H)⁺ 364.1551. found 364.1549.

### Example 145

### 2-(2-((3′-(aminomethyl)-2′-methoxy-[1,1′-biphenyl]-3-yl)methoxy)phenyl)acetic acid

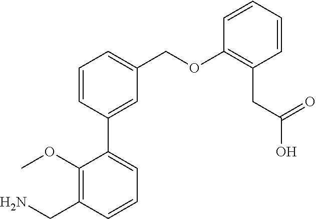 [SMILES: COc1c(CN)cccc1-c1cccc(COc2ccccc2CC(=O)O)c1]

The title compound was synthesized in the same manner as Example 144 from tert-butyl 2-(2-((3-(4,4,5,5-tetramethyl-1,3,2-dioxaborolan-2-yl)benzyl)oxy)phenyl)acetate (Intermediate 78-B) and 3-bromo-2-methoxybenzonitrile (CAS #874472-98-7). ¹H NMR (400 MHz, DMSO-d₆) δ ppm 7.68 (s, 1H) 7.37-7.50 (m, 4H) 7.12-7.28 (m, 4H) 7.03 (d, J=7.82 Hz, 1H) 6.84-6.91 (m, 1H) 5.18 (s, 2H) 3.81 (s, 2H) 3.53 (s, 2H) 3.26 (s, 3H). HRMS calcd. for C₂₃H₂₃NO₄ (M+H)⁺ 378.1716. found 378.1705.

### Example 146

### Example 146-A

### 2-(2-((3-Bromobenzyl)oxy)phenyl)acetamide

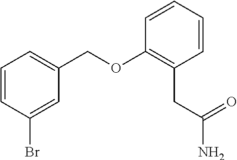 [SMILES: NC(=O)Cc1ccccc1OCc1cccc(Br)c1]

A sealed tube was charged with methyl 2-(2-((3-bromobenzyl)oxy)phenyl)acetate (Example 101-A) (0.65 g, 1.939 mmol), calcium chloride (0.215 g, 1.939 mmol) and NH₃ (7N in MeOH, 12 mL, 84 mmol). The reaction mixture was heated at 80° C. overnight for 18 hours. The reaction was cooled to room temperature and concentrated and absorbed onto silica and purified via FCC (0-100% EA:heptanes) to obtain the title compound. MS (ESI+) m/z 319.9, 321.9 (M+H).

### Example 146-B

### (S)-tert-Butyl (1-(3′-((2-(2-amino-2-oxoethyl)phenoxy)methyl)-[1,1′-biphenyl]-3-yl)-2-hydroxyethyl)carbamate

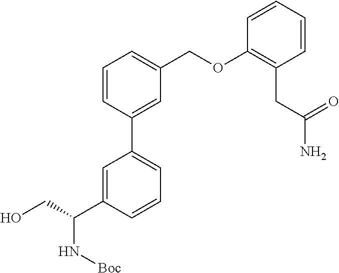 [SMILES: CC(C)(C)OC(=O)N[C@H](CO)c1cccc(-c2cccc(COc3ccccc3CC(N)=O)c2)c1]

The title compound was synthesized in the same manner as Example 141-A from 2-(2-((3-bromobenzyl)oxy)phenyl)acetamide and (S)-tert-butyl (2-hydroxy-1-(3-(4,4,5,5-tetramethyl-1,3,2-dioxaborolan-2-yl)phenyl)ethyl)carbamate (Intermediate 34-B). MS (ESI+) m/z 477.2 (M+H).

### Example 146-C

### (S)-2-(2-((3′-(1-Amino-2-hydroxyethyl)-[1,1′-biphenyl]-3-yl)methoxy)phenyl)acetamide

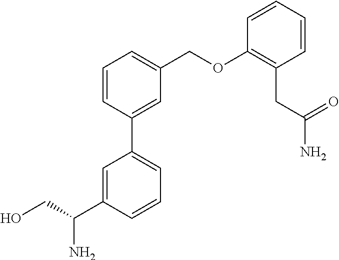 [SMILES: NC(=O)Cc1ccccc1OCc1cccc(-c2cccc([C@H](N)CO)c2)c1]

The title compound was synthesized in the same manner as Example 114-B starting with (S)-tert-butyl (1-(3′-((2-(2-amino-2-oxoethyl)phenoxy)methyl)-[1,1′-biphenyl]-3-yl)-2-hydroxyethyl)carbamate. ¹H NMR (400 MHz, DMSO-d₆) δ ppm 7.77 (s, 1H) 7.69 (s, 1H) 7.60 (td, J=4.46, 1.83 Hz, 1H) 7.53 (dt, J=7.49, 1.51 Hz, 1H) 7.47 (d, J=4.77 Hz, 2H) 7.32-7.42 (m, 2H) 7.26 (br. s., 1H) 7.17-7.23 (m, 2H) 7.02-7.07 (m, 1H) 6.86-6.95 (m, 2H) 5.19 (s, 2H) 4.78 (t, J=5.38 Hz, 1H) 3.94 (dd, J=7.64, 4.83 Hz, 1H) 3.51 (dt, J=10.30, 5.06 Hz, 1H) 3.46 (s, 2H). HRMS calcd. for C₂₃H₂₄N₂O₃ (M+H)⁺ 377.1857. found 377.1865.

### Example 147

### Example 147-A

### (S)-tert-Butyl 2-(2-((3′-(1-((tert-butoxycarbonyl)amino)-2-hydroxyethyl)-5-(cyclopropylmethoxy)-[1,1′-biphenyl]-3-yl)methoxy)phenyl)acetate

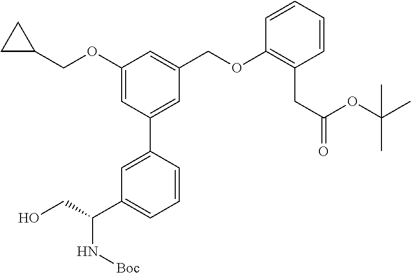 [SMILES: CC(C)(C)OC(=O)Cc1ccccc1OCc1cc(OCC2CC2)cc(-c2cccc([C@@H](CO)NC(=O)OC(C)(C)C)c2)c1]

The title compound was synthesized in the same manner as Example 141-A from tert-butyl 2-(2-((3-bromo-5-(cyclopropylmethoxy)benzyl)oxy)phenyl)acetate (Intermediate 89) and (S)-tert-butyl (2-hydroxy-1-(3-(4,4,5,5-tetramethyl-1,3,2-dioxaborolan-2-yl)phenyl)ethyl)carbamate (Intermediate 34-B). MS (ESI+) m/z 504.3 (M−Boc+H).

### Example 147-B

### (S)-2-(2-((3′-(1-Amino-2-hydroxyethyl)-5-(cyclopropylmethoxy)-[1,1′-biphenyl]-3-yl)methoxy)phenyl)acetic acid

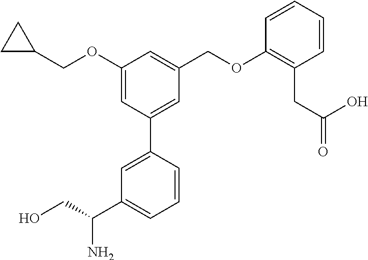 [SMILES: N[C@H](CO)c1cccc(-c2cc(COc3ccccc3CC(=O)O)cc(OCC3CC3)c2)c1]

The title compound was synthesized in the same manner as Example 114-B from (S)-tert-butyl 2-(2-((3′-(1-((tert-butoxycarbonyl)amino)-2-hydroxyethyl)-5-(cyclopropylmethoxy)-[1,1′-biphenyl]-3-yl)methoxy)phenyl)acetate. ¹H NMR (400 MHz, DMSO-d₆) δ ppm 8.13 (s, 1H) 7.62 (d, J=7.95 Hz, 1H) 7.50 (s, 1H) 7.27-7.36 (m, 1H) 7.22 (d, J=7.70 Hz, 1H) 7.09 (s, 1H) 6.99-7.06 (m, 2H) 6.92 (s, 1H) 6.85 (d, J=7.95 Hz, 1H) 6.67-6.78 (m, 1H) 5.08 (s, 2H) 4.08 (dd, J=6.85, 4.52 Hz, 1H) 3.85 (d, J=6.97 Hz, 2H) 3.56-3.68 (m, 2H) 3.25-3.44 (m, 2H) 1.19 (s, 1H) 0.49-0.56 (m, 2H) 0.28 (dd, J=4.77, 1.34 Hz, 2H). HRMS calcd. for C₂₇H₂₉NO₅ (M+H)⁺448.2109. found 448.2124.

### Example 148

### (R)-2-(2-((3′-(1-Aminoethyl)-5-(cyclopropylmethoxy)-2′-fluoro-[1,1′-biphenyl]-3-yl)methoxy)phenyl)acetic acid

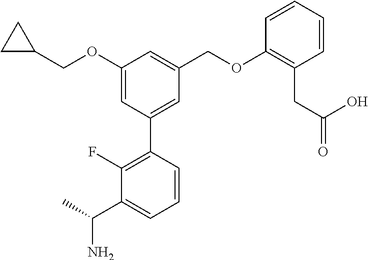 [SMILES: C[C@@H](N)c1cccc(-c2cc(COc3ccccc3CC(=O)O)cc(OCC3CC3)c2)c1F]

The title compound was synthesized in the same manner as Example 147 starting with tert-butyl 2-(2-((3-bromo-5-(cyclopropylmethoxy)benzyl)oxy)phenyl)acetate (Intermediate 89) and (R)-tert-butyl (1-(2-fluoro-3-(4,4,5,5-tetramethyl-1,3,2-dioxaborolan-2-yl)phenyl)ethyl)carbamate (Intermediate 27-B). ¹H NMR (400 MHz, DMSO-d₆) δ ppm 7.49-7.59 (m, 1H) 7.37 (td, J=7.52, 1.71 Hz, 1H) 7.13-7.28 (m, 4H) 6.94-7.06 (m, 3H) 6.88 (td, J=7.37, 0.79 Hz, 1H) 5.14 (s, 2H) 4.35 (q, J=6.60 Hz, 1H) 3.81-3.93 (m, 2H) 3.54 (s, 2H) 1.33 (d, J=6.72 Hz, 3H) 1.17-1.29 (m, 1H) 0.51-0.62 (m, 2H) 0.29-0.39 (m, 2H). HRMS calcd. for C₂₇H₂₈FNO₄ (M+H)⁺ 450.2072. found 450.2081.

### Example 149

### 2-(2-((3′-(Aminomethyl)-2′-fluoro-6-methyl-[1,1′-biphenyl]-3-yl)methoxy)phenyl)acetic acid

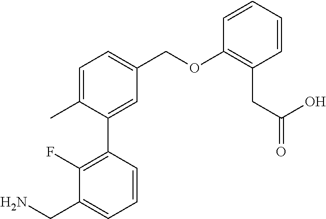 [SMILES: Cc1ccc(COc2ccccc2CC(=O)O)cc1-c1cccc(CN)c1F]

The title compound was synthesized in the same manner as Example 147 starting from tert-butyl 2-(2-((3-bromo-4-methylbenzyl)oxy)phenyl)acetate (Intermediate 66) and (3-(aminomethyl)-2-fluorophenyl)boronic acid hydrochloride (CAS #1072946-44-1). ¹H NMR (400 MHz, DMSO-d₆) δ ppm 7.49 (td, J=7.18, 1.77 Hz, 1H) 7.39 (d, J=1.59 Hz, 1H) 7.27-7.34 (m, 2H) 7.13-7.26 (m, 4H) 7.00 (d, J=7.95 Hz, 1H) 6.86 (td, J=7.34, 0.86 Hz, 1H) 5.10 (s, 2H) 3.82 (br. s., 2H) 3.48 (s, 2H) 2.14 (s, 3H). HRMS calcd. for C₂₃H₂₂FNO₃ (M+H)⁺ 380.1650. found 380.1662.

### Example 150

### Example 150-A

### (R)-tert-Butyl 2-(2-((3′-(1-((tert-butoxycarbonyl)amino)ethyl)-5-chloro-2′-fluoro-[1,1′-biphenyl]-3-yl)methoxy)phenyl)acetate

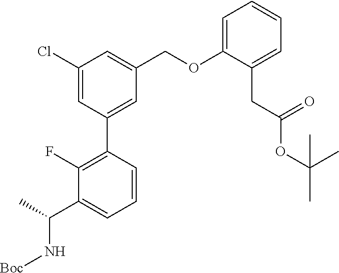 [SMILES: C[C@@H](NC(=O)OC(C)(C)C)c1cccc(-c2cc(Cl)cc(COc3ccccc3CC(=O)OC(C)(C)C)c2)c1F]

The title compound was synthesized in the same manner as Example 141-A starting with tert-butyl 2-(2-((3-bromo-5-chlorobenzyl)oxy)phenyl)acetate (Intermediate 53) and (R)-tert-butyl (1-(2-fluoro-3-(4,4,5,5-tetramethyl-1,3,2-dioxaborolan-2-yl)phenyl)ethyl)carbamate (Intermediate 27-B). MS (ESI−) m/z 569.1 (M−H).

### Example 150-B

### (R)-tert-Butyl 2-(2-((3′-(1-((tert-butoxycarbonyl)amino)ethyl)-5-((cyclopropylmethyl)amino)-2′-fluoro-[1,1′-biphenyl]-3-yl)methoxy)phenyl)acetate

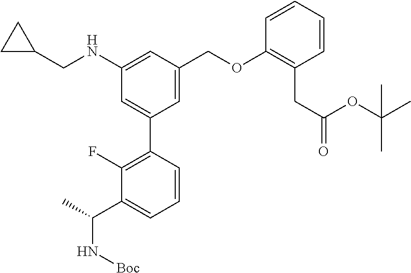 [SMILES: C[C@@H](NC(=O)OC(C)(C)C)c1cccc(-c2cc(COc3ccccc3CC(=O)OC(C)(C)C)cc(NCC3CC3)c2)c1F]

To (R)-tert-butyl 2-(2-((3′-(1-((tert-butoxycarbonyl)amino)ethyl)-5-chloro-2′-fluoro-[1,1′-biphenyl]-3-yl)methoxy)phenyl)acetate (111 mg, 0.195 mmol) was added cyclopropylmethanamine (0.034 mL, 0.389 mmol), BrettPhos palladacycle (7.78 mg, 9.74 μmol) and Cs₂CO₃ (317 mg, 0.974 mmol) in CH₃CN (1.9 mL) and heated at 110° C. for 60 min. The reaction was cooled to room temperature concentrated and absorbed onto silica and purified via FCC (0-30% EA:heptanes) to obtain the title compound. MS (ESI+) m/z 549.4 (M−tBu+H).

### Example 150-C

### (R)-2-(2-((3′-(1-Aminoethyl)-5-((cyclopropylmethyl)amino)-2′-fluoro-[1,1′-biphenyl]-3-yl)methoxy)phenyl)acetic acid

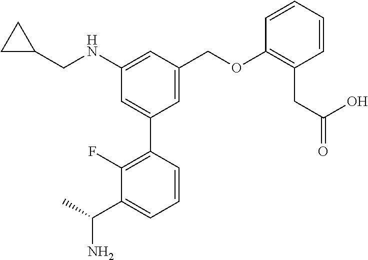 [SMILES: C[C@@H](N)c1cccc(-c2cc(COc3ccccc3CC(=O)O)cc(NCC3CC3)c2)c1F]

The title compound was synthesized in the same manner as Example 114-B from (R)-tert-butyl 2-(2-((3′-(1-((tert-butoxycarbonyl)amino)ethyl)-5-((cyclopropylmethyl)amino)-2′-fluoro-[1,1′-biphenyl]-3-yl)methoxy)phenyl)acetate. ¹H NMR (400 MHz, DMSO-d₆) δ ppm 7.52 (td, J=7.21, 1.59 Hz, 1H) 7.30 (td, J=7.49, 1.77 Hz, 1H) 7.14-7.24 (m, 3H) 7.01 (d, J=7.95 Hz, 1H) 6.87 (td, J=7.37, 0.92 Hz, 1H) 6.78 (s, 1H) 6.61-6.70 (m, 2H) 5.79 (br. s., 1H) 5.04 (s, 2H) 4.27-4.39 (m, 1H) 3.54 (s, 2H) 2.88-2.97 (m, 2H) 1.31 (d, J=6.72 Hz, 3H) 0.98-1.11 (m, 1H) 0.41-0.52 (m, 2H) 0.18-0.26 (m, 2H). HRMS calcd. for C₂₇H₂₉FN₂O₃(M+H)⁺ 449.2242. found 449.2240.

### Example 151

### (R)-2-(2-((3′-(1-aminoethyl)-5-(ethylamino)-2′-fluoro-[1,1′-biphenyl]-3-yl)methoxy)phenyl)acetic acid

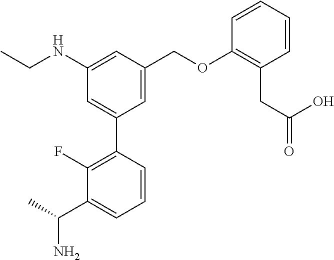 [SMILES: CCNc1cc(COc2ccccc2CC(=O)O)cc(-c2cccc([C@@H](C)N)c2F)c1]

The title compound was synthesized in the same manner as Examples 150-B and 150-C using ethanamine in place of cyclopropylmethanamine. ¹H NMR (400 MHz, DMSO-d₆) δ ppm 7.52 (td, J=7.21, 1.59 Hz, 1H) 7.30 (td, J=7.49, 1.77 Hz, 1H) 7.13-7.25 (m, 3H) 7.01 (d, J=8.07 Hz, 1H) 6.87 (td, J=7.40, 0.86 Hz, 1H) 6.78 (s, 1H) 6.64 (d, J=7.58 Hz, 2H) 5.66 (br. s., 1H) 5.05 (s, 2H) 4.34 (d, J=6.60 Hz, 1H) 3.54 (s, 2H) 3.07 (q, J=6.64 Hz, 2H) 1.31 (d, J=6.60 Hz, 3H) 1.17 (t, J=7.09 Hz, 3H). HRMS calcd. for C₂₅H₂₇FN₂O₃(M+H)⁺ 423.2069. found 423.2084.

### Example 152

### (R)-2-(2-((3′-(1-aminoethyl)-2′-fluoro-5-(isopropylamino)-[1,1′-biphenyl]-3-yl)methoxy)phenyl)acetic acid

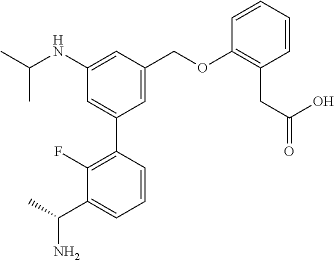 [SMILES: CC(C)Nc1cc(COc2ccccc2CC(=O)O)cc(-c2cccc([C@@H](C)N)c2F)c1]

The title compound was synthesized in the same manner as Examples 150-B and 150-C using propan-2-amine in place of cyclopropylmethanamine. ¹H NMR (400 MHz, DMSO-d₆) δ ppm 7.52 (td, J=7.27, 1.59 Hz, 1H) 7.29 (td, J=7.46, 1.83 Hz, 1H) 7.15-7.24 (m, 3H) 7.02 (d, J=7.95 Hz, 1H) 6.83-6.91 (m, 1H) 6.75 (s, 1H) 6.63 (d, J=4.89 Hz, 2H) 5.51 (d, J=7.70 Hz, 1H) 5.04 (s, 2H) 4.33 (q, J=6.56 Hz, 1H) 3.49-3.65 (m, 3H) 1.31 (d, J=6.60 Hz, 3H) 1.14 (d, J=6.36 Hz, 6H). HRMS calcd. for C₂₆H₂₉FN₂O₃(M+H)⁺ 437.2231. found 437.2240.

### Example 153

### 2-(2-((3′-((R)-1-Aminoethyl)-2′-fluoro-5-((((R)-tetrahydrofuran-2-yl)methyl)amino)-[1,1′-biphenyl]-3-yl)methoxy)phenyl)acetic acid

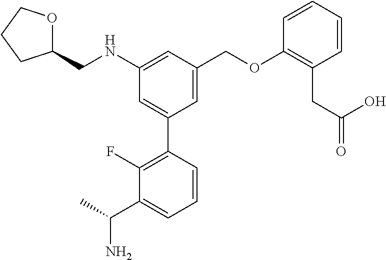 [SMILES: C[C@@H](N)c1cccc(-c2cc(COc3ccccc3CC(=O)O)cc(NC[C@H]3CCCO3)c2)c1F]

The title compound was synthesized in the same manner as Examples 150-B and 150-C starting with (R)-(tetrahydrofuran-2-yl)methanamine (CAS #7202-43-9) in place of cyclopropylmethanamine. ¹H NMR (400 MHz, DMSO-d₆) δ ppm 7.52 (td, J=7.21, 1.59 Hz, 1H) 7.30 (td, J=7.49, 1.77 Hz, 1H) 7.14-7.25 (m, 3H) 7.01 (d, J=7.95 Hz, 1H) 6.87 (td, J=7.37, 0.79 Hz, 1H) 6.79 (s, 1H) 6.65-6.72 (m, 2H) 5.72 (t, J=5.62 Hz, 1H) 5.04 (s, 2H) 4.34 (q, J=6.60 Hz, 1H) 3.95-4.04 (m, 1H) 3.74-3.82 (m, 1H) 3.59-3.68 (m, 1H) 3.54 (s, 2H) 3.03-3.19 (m, 2H) 1.90-2.01 (m, 1H) 1.74-1.90 (m, 2H) 1.52-1.65 (m, 1H) 1.32 (d, J=6.72 Hz, 3H). HRMS calcd. for C₂₈H₃₁FN₂O₄(M+H)⁺ 479.2343. found 479.2346.

### Example 154

### Example 154-A

### (±)-(5-Bromo-2-fluoro-3-(((tetrahydrofuran-2-yl)methyl)amino)phenyl)methanol

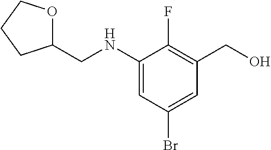 [SMILES: OCc1cc(Br)cc(NCC2CCCO2)c1F]

A mixture of (3-amino-5-bromo-2-fluorophenyl)methanol (Intermediate 79) (400 mg, 1.818 mmol) and (±)-tetrahydrofuran-2-carbaldehyde (CAS #7681-84-7) (218 mg, 2.181 mmol) in DCE (15 mL) was stirred at rt for 45 min. Na(AcO)₃BH (578 mg, 2.73 mmol) was added portionwise and the resulting mixture was stirred at rt overnight. Sat. aq. NH₄Cl solution was added and the mixture was extracted with EtOAc. The combined organics were washed with brine, dried (Na₂SO₄) and concentrated. The residue was purified by flash chromatography (EtOAc-heptane 0-100%) to provide a mixture of the title compound and recovered starting material (3-amino-5-bromo-2-fluorophenyl)methanol, which was carried onto the next step without further purification. MS (ESI) m/z 303.9, 305.9 (M+H).

### Example 154-B

### tert-Butyl 2-(2-((3-amino-5-bromo-2-fluorobenzyl)oxy)phenyl)acetate and (±)-tert-butyl 2-(2-((5-bromo-2-fluoro-3-(((tetrahydrofuran-2-yl)methyl)amino)benzyl)oxy)phenyl)acetate

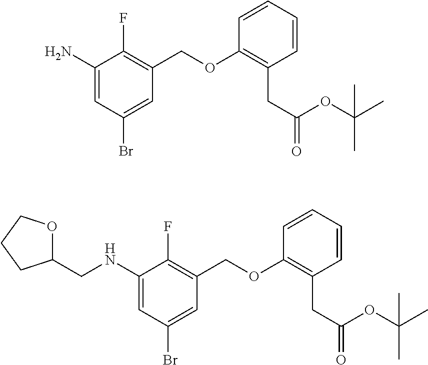 [SMILES: CC(C)(C)OC(=O)Cc1ccccc1OCc1cc(Br)cc(N)c1F ; CC(C)(C)OC(=O)Cc1ccccc1OCc1cc(Br)cc(NCC2CCCO2)c1F]

DIAD (0.384 mL, 1.973 mmol) was added dropwise to a solution of a mixture of (5-bromo-2-fluoro-3-(((tetrahydrofuran-2-yl)methyl)amino)phenyl)methanol and (3-amino-5-bromo-2-fluorophenyl)methanol (431 mg), tert-butyl 2-(2-hydroxyphenyl)acetate (Intermediate 21) and PPh₃ (517 mg, 1.973 mmol) in THF (10 mL) at 0° C. The resulting solution was allowed to warm to room temperature and then stirred overnight. Sat. aq. NH₄Cl was added and the mixture was extracted with EtOAc. The combined organics were washed with brine, dried (Na₂SO₄) and concentrated. The residue was purified by flash chromatography (EtOAc-heptane 0-50%) to provide a mixture of the title compounds. tert-Butyl 2-(2-((3-amino-5-bromo-2-fluorobenzyl)oxy)phenyl)acetate, MS (ESI+) m/z 353.9, 355.9 (M−tBu+H) and tert-butyl 2-(2-((5-bromo-2-fluoro-3-(((tetrahydrofuran-2-yl)methyl)amino)benzyl)oxy)phenyl)acetate, MS (ESI+) m/z 494.1, 496.1 (M+H).

### Example 154-C

### a) 2-(2-((5-Amino-3′-(aminomethyl)-4-fluoro-[1,1′-biphenyl]-3-yl)methoxy)phenyl)acetic acid and b) (±)-2-(2-((3′-(aminomethyl)-4-fluoro-5-(((tetrahydrofuran-2-yl)methyl)amino)-[1,1′-biphenyl]-3-yl)methoxy)phenyl)acetic acid

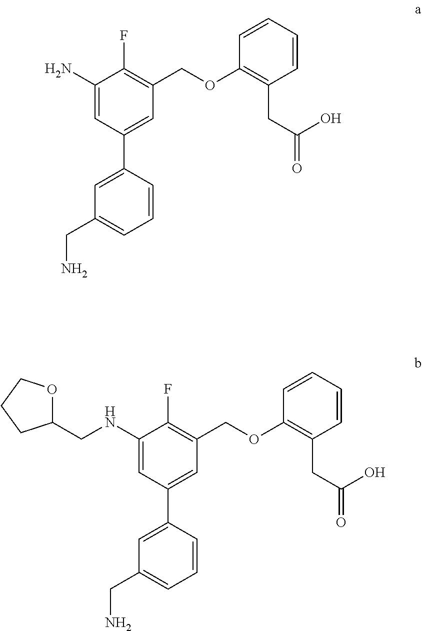 [SMILES: NCc1cccc(-c2cc(N)c(F)c(COc3ccccc3CC(=O)O)c2)c1 ; NCc1cccc(-c2cc(COc3ccccc3CC(=O)O)c(F)c(NCC3CCCO3)c2)c1]

A mixture of tert-butyl 2-(2-((3-amino-5-bromo-2-fluorobenzyl)oxy)phenyl)acetate and tert-butyl 2-(2-((5-bromo-2-fluoro-3-(((tetrahydrofuran-2-yl)methyl)amino)benzyl)oxy)phenyl)acetate (113 mg), (3-(((tert-butoxycarbonyl)amino)methyl)phenyl)boronic acid (CAS #199609-62-6) (92 mg, 0.366 mmol), PdCl₂(dppf).CH₂Cl₂ adduct (CAS #95464-05-4) (19.90 mg, 0.024 mmol), and 2M aq. K₃PO₄ (0.366 mL, 0.731 mmol) in CH₃CN (2 mL) was heated at 110° C. in a microwave for 1 hr. The mixture was partitioned between EtOAc and sat. NH₄Cl. The aqueous layer was extracted with EtOAc. The combined organics were washed with brine, dried (Na₂SO₄) and concentrated. The residue was purified by flash chromatography (EtOAc-heptane 0-60%) and the resulting material was dissolved in dichloromethane (2 mL) and TFA (0.5 mL, 6.49 mmol) was added. The mixture was stirred at rt overnight. The mixture was concentrated and the residue was purified by preparative HPLC (Method B) to provide the title compounds.

a): ¹H NMR (400 MHz, METHANOL-d₄) δ ppm 7.97 (s, 1H) 7.59 (d, J=7.83 Hz, 1H) 7.41 (t, J=7.71 Hz, 1H) 7.25-7.35 (m, 2H) 7.13-7.22 (m, 2H) 7.09 (dd, J=8.21, 2.27 Hz, 1H) 6.81-6.99 (m, 2H) 5.21 (s, 2H) 4.13 (s, 2H) 3.62 (s, 2H). HRMS calcd. for C₂₂H₂₁FN₂O₃ (M+H)⁺ 381.1614. found 381.1616.

b) (mixture of diastereomers): ¹H NMR (400 MHz, METHANOL-d₄) δ ppm 7.99 (s, 1H) 7.63 (d, J=8.08 Hz, 1H) 7.36-7.47 (m, 1H) 7.24-7.34 (m, 2H) 7.12-7.23 (m, 2H) 6.96-7.05 (m, 1H) 6.84-6.95 (m, 2H) 5.21 (s, 2H) 4.17 (qd, J=6.88, 4.36 Hz, 1H) 4.11 (s, 2H) 3.91 (dt, J=8.05, 6.77 Hz, 1H) 3.74-3.85 (m, 1H) 3.61 (s, 2H) 3.34-3.43 (m, 1H) 3.21-3.28 (m, 1H) 2.02-2.19 (m, 1H) 1.87-2.02 (m, 2H) 1.67-1.82 (m, 1H). HRMS calcd. for C₂₇H₂₉FN₂O₄(M+H)⁺ 465.2190. found 465.2184.

### Example 155

### a) (S)-2-(2-((5-Amino-3′-(1-amino-2-hydroxyethyl)-4-fluoro-[1,1′-biphenyl]-3-yl)methoxy)phenyl)acetic acid and b) 2-(2-((3′-((S)-1-Amino-2-hydroxyethyl)-4-fluoro-5-(((tetrahydrofuran-2-yl)methyl)amino)-[1,1′-biphenyl]-3-yl)methoxy)phenyl)acetic acid (mixture of diastereomers)

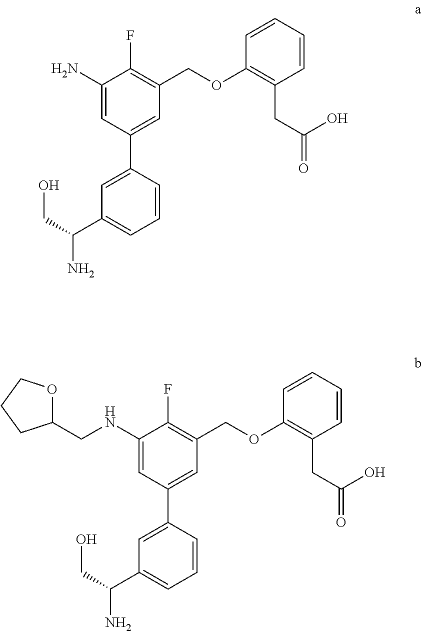 [SMILES: Nc1cc(-c2cccc([C@H](N)CO)c2)cc(COc2ccccc2CC(=O)O)c1F ; N[C@H](CO)c1cccc(-c2cc(COc3ccccc3CC(=O)O)c(F)c(NCC3CCCO3)c2)c1]

The title compounds were synthesized in the same manner as Examples 154-C using (S)-tert-butyl (2-hydroxy-1-(3-(4,4,5,5-tetramethyl-1,3,2-dioxaborolan-2-yl)phenyl)ethyl)carbamate (Intermediate 34-B) instead of (3-(((tert-butoxycarbonyl)amino)methyl)phenyl)boronic acid.

a): ¹H NMR (400 MHz, METHANOL-d₄) δ ppm 8.08 (s, 1H) 7.60 (d, J=7.96 Hz, 1H) 7.37-7.48 (m, 2H) 7.27 (d, J=7.71 Hz, 1H) 7.12-7.22 (m, 2H) 7.09 (dd, J=8.21, 2.40 Hz, 1H) 6.94 (d, J=7.96 Hz, 1H) 6.87 (td, J=7.39, 0.88 Hz, 1H) 5.14-5.31 (m, 2H) 4.32 (dd, J=8.84, 4.55 Hz, 1H) 3.89-3.99 (m, 1H) 3.81-3.89 (m, 1H) 3.50-3.69 (m, 2H). HRMS calcd. for C₂₃H₂₃FN₂O₄(M+H)⁺ 411.1720. found 411.1715.

b): (mixture of diastereomers): ¹H NMR (400 MHz, METHANOL-d₄) δ ppm 8.08 (s, 1H) 7.64 (d, J=8.08 Hz, 1H) 7.42 (t, J=7.71 Hz, 1H) 7.36 (dd, J=6.13, 2.08 Hz, 1H) 7.28 (d, J=7.71 Hz, 1H) 7.12-7.23 (m, 2H) 7.01 (dd, J=8.02, 2.21 Hz, 1H) 6.94 (d, J=7.58 Hz, 1H) 6.87 (td, J=7.42, 0.95 Hz, 1H) 5.16-5.33 (m, 2H) 4.33 (dd, J=8.91, 4.61 Hz, 1H) 4.10-4.23 (m, 1H) 3.74-4.03 (m, 4H) 3.49-3.72 (m, 2H) 3.21-3.44 (m, 2H) 2.02-2.18 (m, 1H) 1.86-2.02 (m, 2H) 1.65-1.83 (m, 1H). HRMS calcd. for C₂₈H₃₁FN₂O₅(M+H)⁺ 495.2295. found 495.2283.

### Example 156

### Example 156-A

### (±)-tert-Butyl 2-(2-((3′-(((tert-butoxycarbonyl)amino)methyl)-6-fluoro-5-(((tetrahydrofuran-2-yl)methyl)amino)-[1,1′-biphenyl]-3-yl)methoxy)phenyl)acetate

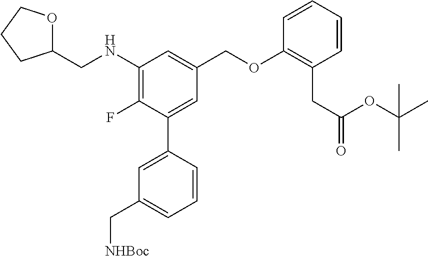 [SMILES: CC(C)(C)OC(=O)Cc1ccccc1OCc1cc(NCC2CCCO2)c(F)c(-c2cccc(CNC(=O)OC(C)(C)C)c2)c1]

A mixture of tert-butyl 2-(2-((3-bromo-4-fluoro-5-(((tetrahydrofuran-2-yl)methyl)amino)benzyl)oxy)phenyl)acetate (Intermediate 80-C) (68 mg, 0.138 mmol), (3-(((tert-butoxycarbonyl)amino)methyl)phenyl)boronic acid (CAS #199609-62-6) (51.8 mg, 0.206 mmol), PdCl₂(dppf).CH₂Cl₂ adduct (CAS #95464-05-4) (11.23 mg, 0.014 mmol) and 2M aq. K₃PO₄ (0.206 mL, 0.413 mmol) in CH₃CN (1.5 mL) was heated in a microwave at 110° C. for 1 hr. The residue was partitioned between EtOAc and a sat. aq. solution of NH₄Cl. The aqueous layer was extracted with EtOAc. The combined organics were washed with brine, dried (Na₂SO₄) and concentrated. The residue was purified by flash chromatography (EtOAc-heptane 0-50%) to provide the title compound. MS (ESI+) m/z 621.4 (M+H).

### Example 156-B

### (±)-2-(2-((3′-(Aminomethyl)-6-fluoro-5-(((tetrahydrofuran-2-yl)methyl)amino)-[1,1′-biphenyl]-3-yl)methoxy)phenyl)acetic acid

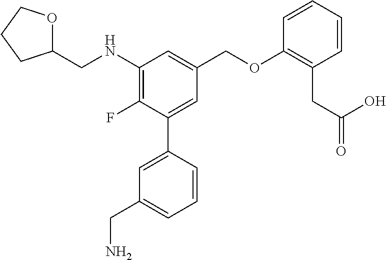 [SMILES: NCc1cccc(-c2cc(COc3ccccc3CC(=O)O)cc(NCC3CCCO3)c2F)c1]

TFA (1 mL, 12.98 mmol) was added to a solution of tert-butyl 2-(2-((3′-(((tert-butoxycarbonyl)amino)methyl)-6-fluoro-5-(((tetrahydrofuran-2-yl)methyl)amino)-[1,1′-biphenyl]-3-yl)methoxy)phenyl)acetate (77 mg, 0.124 mmol) in DCM (2 mL) at rt. The mixture was stirred at rt overnight. The mixture was concentrated and the residue was purified by preparative HPLC (Method B). ¹H NMR (400 MHz, METHANOL-d₄) δ ppm 7.93 (s, 1H) 7.69 (dt, J=7.86, 1.50 Hz, 1H) 7.35-7.45 (m, 1H) 7.28-7.35 (m, 1H) 7.11-7.24 (m, 3H) 6.93 (d, J=7.83 Hz, 1H) 6.75-6.90 (m, 2H) 5.09 (s, 2H) 4.09-4.26 (m, 3H) 3.91 (dt, J=8.18, 6.71 Hz, 1H) 3.73-3.83 (m, 1H) 3.53-3.59 (m, 2H) 3.20-3.41 (m, 2H) 2.02-2.15 (m, 1H) 1.86-2.01 (m, 2H) 1.73 (ddt, J=11.95, 8.54, 7.06, 7.06 Hz, 1H). HRMS calcd. for C₂₇H₂₉FN₂O₄(M+H)⁺ 465.2190. found 465.2178.

### Example 157

### 2-(2-((3′-((S)-1-Amino-2-hydroxyethyl)-6-fluoro-5-(((tetrahydrofuran-2-yl)methyl)amino)-[1,1′-biphenyl]-3-yl)methoxy)phenyl)acetic acid (mixture of diastereomers)

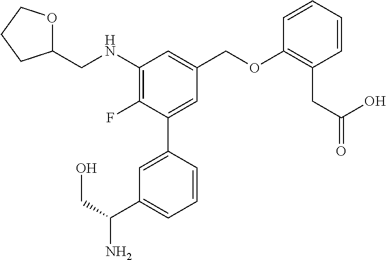 [SMILES: N[C@H](CO)c1cccc(-c2cc(COc3ccccc3CC(=O)O)cc(NCC3CCCO3)c2F)c1]

The title compound was synthesized as described in Example 156 starting with (S)-tert-butyl (2-hydroxy-1-(3-(4,4,5,5-tetramethyl-1,3,2-dioxaborolan-2-yl)phenyl)ethyl)carbamate (Intermediate 34-B) instead of (3-(((tert-butoxycarbonyl)amino)methyl)phenyl)boronic acid. ¹H NMR (400 MHz, METHANOL-d₄) δ ppm 7.96 (s, 1H) 7.71 (dt, J=7.86, 1.50 Hz, 1H) 7.37-7.48 (m, 1H) 7.30-7.37 (m, 1H) 7.11-7.21 (m, 3H) 6.92-7.01 (m, 1H) 6.85 (ddd, J=14.87, 7.48, 1.39 Hz, 2H) 5.00-5.14 (m, 2H) 4.33 (dd, J=8.72, 4.67 Hz, 1H) 4.11-4.24 (m, 1H) 3.83-4.01 (m, 3H) 3.74-3.83 (m, 1H) 3.47-3.64 (m, 2H) 3.20-3.41 (m, 2H) 2.02-2.15 (m, 1H) 1.88-2.02 (m, 2H) 1.67-1.81 (m, 1H). HRMS calcd. for C₂₈H₃₁FN₂O₅(M+H)⁺ 495.2295. found 495.2274.

### Example 158

### Example 158-A

### tert-Butyl 2-(2-((3-(4-cyanopyrimidin-2-yl)benzyl)oxy)phenyl)acetate

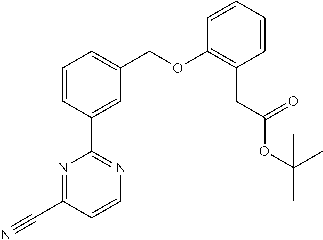 [SMILES: CC(C)(C)OC(=O)Cc1ccccc1OCc1cccc(-c2nccc(C#N)n2)c1]

In a vial with a stir bar was placed tert-butyl 2-(2-((3-(4,4,5,5-tetramethyl-1,3,2-dioxaborolan-2-yl)benzyl)oxy)phenyl)acetate (Intermediate 78-B) (0.131 g, 0.309 mmol) and 2-chloropyrimidine-4-carbonitrile (CAS #75833-38-4) (0.054 g, 0.386 mmol) in DMF (2.8 ml) and water (0.3 ml). Then, 2M aq. K₃PO₄ (0.617 ml, 1.235 mmol) and PdCl₂(dppf).CH₂Cl₂ adduct (CAS #95464-05-4) (0.013 g, 0.015 mmol) were added. The vial was sealed and the reaction was heated at 45° C. overnight. The reaction was diluted with water and EA and the EA layer was removed, dried and purified via flash chromatography (0-100% EA:heptanes) to obtain the title compound. MS (ESI+) m/z 401.9 (M+H).

### Example 158-B

### tert-Butyl 2-(2-((3-(4-(aminomethyl)pyrimidin-2-yl)benzyl)oxy)phenyl)acetate

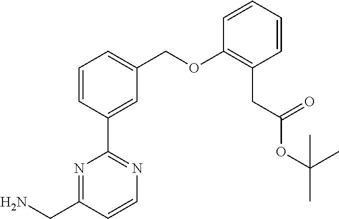 [SMILES: CC(C)(C)OC(=O)Cc1ccccc1OCc1cccc(-c2nccc(CN)n2)c1]

To tert-butyl 2-(2-((3-(4-cyanopyrimidin-2-yl)benzyl)oxy)phenyl)acetate (77 mg, 0.192 mmol) in MeOH (3.8 mL) was added 10% Pd/C (10.21 mg, 9.59 μmol) and the mixture was evacuated and filled with hydrogen from a balloon and then stirred for 2 hours. The reaction was filtered and washed with MeOH, concentrated and absorbed onto silica to purify via FCC (0-20% MeOH containing 10% ammonium hydroxide:DCM) to obtain the title compound. MS (ESI+) m/z 406.0 (M+H).

### Example 158-C

### 2-(2-((3-(4-(Aminomethyl)pyrimidin-2-yl)benzyl)oxy)phenyl)acetic acid

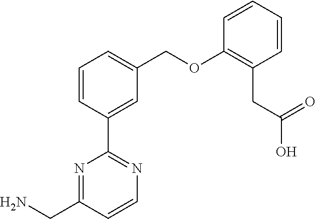 [SMILES: NCc1ccnc(-c2cccc(COc3ccccc3CC(=O)O)c2)n1]

The title compound was synthesized in the same manner as Example 141-B from tert-butyl 2-(2-((3-(4-(aminomethyl)pyrimidin-2-yl)benzyl)oxy)phenyl)acetate. ¹H NMR (400 MHz, DMSO-d₆) δ ppm 8.71-8.92 (m, 2H) 8.33 (d, J=7.58 Hz, 1H) 7.48-7.60 (m, 2H) 7.43 (d, J=5.14 Hz, 1H) 7.13 (dd, J=7.58, 1.83 Hz, 2H) 6.95 (d, J=7.95 Hz, 1H) 6.84 (t, J=7.27 Hz, 1H) 5.22 (s, 2H) 4.05 (s, 2H) 3.39-3.48 (m, 2H). HRMS calcd. for C₂₀H₁₉N₃O₃ (M+H)⁺ 350.1521. found 350.1505.

### Example 159

### 2-(2-((3′-(Aminomethyl)-[1,1′-biphenyl]-3-yl)methoxy)phenyl)acetic acid

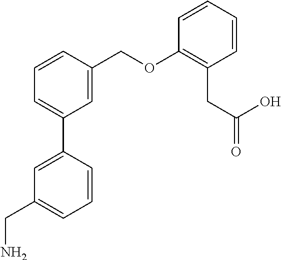 [SMILES: NCc1cccc(-c2cccc(COc3ccccc3CC(=O)O)c2)c1]

A microwave vial was charged with methyl 2-(2-((3-bromobenzyl)oxy)phenyl)acetate (Example 101-A) (109 mg, 0.325 mmol), PdCl₂(dppf).CH₂Cl₂ (13.28 mg, 0.016 mmol) and (3-(aminomethyl)phenyl)boronic acid hydrochloride (CAS #146285-80-5) (76 mg, 0.406 mmol). Acetonitrile (1.3 mL), 2M aqueous K₃PO₄ (0.488 mL, 0.976 mmol) and water (0.130 mL) were added, the vial was flushed with nitrogen, sealed and heated in the microwave for 60 min at 110° C. THF (4 mL) and 2M aqueous LiOH (0.813 mL, 1.626 mmol) were added and the mixture was heated at 50° C. for 4 hours. Additional 2M aqueous LiOH (0.813 mL, 1.626 mmol) was added and the reaction was heated at 50° C. overnight. The mixture was acidified to pH 4-5 with 2N HCl, extracted with ethyl acetate, washed with water and brine, dried with sodium sulfate, filtered and concentrated. The crude was purified by preparative HPLC (method B) to provide the title compound. ¹H NMR (400 MHz, ACETONITRILE-d₃) δ ppm 7.89-7.84 (m, 1H), 7.84-7.80 (m, 1H), 7.62 (d, J=8.0 Hz, 1H), 7.57 (d, J=7.2 Hz, 1H), 7.40 (dt, J=1.8, 7.6 Hz, 2H), 7.27-7.19 (m, 2H), 7.16 (d, J=7.5 Hz, 2H), 6.88 (dt, J=0.9, 7.4 Hz, 1H), 6.83 (d, J=7.8 Hz, 1H), 5.00 (s, 2H), 3.92 (s, 2H), 3.57 (s, 2H). HRMS calcd. for C₂₂H₂₁NO₃ (M+H)⁺ 348.1600. found 348.1589.

### Example 160

### 2-(2-((6-(3-(aminomethyl)phenyl)pyridin-2-yl)methoxy)phenyl)acetic acid

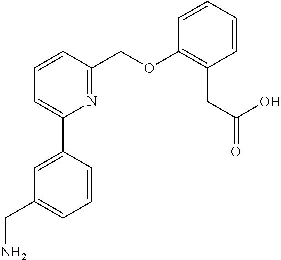 [SMILES: NCc1cccc(-c2cccc(COc3ccccc3CC(=O)O)n2)c1]

Methyl 2-(2-((6-(3-(((tert-butoxycarbonyl)amino)methyl)phenyl)pyridin-2-yl)methoxy)phenyl)acetate (Intermediate 84-A) (167 mg, 0.361 mmol) and 2M aqueous LiOH (0.903 mL, 1.805 mmol) were heated in THF (2 mL) at 50° C. overnight. The reaction was acidified with 1N HCl, extracted with ethyl acetate, washed with brine, dried over sodium sulfate and concentrated. The crude product was dissolved in 1:1 TFA/DCM (2 mL) and stirred at room temperature for two hours. The reaction was concentrated and purified by preparative HPLC (Method B) to provide the title compound. ¹H NMR (400 MHz, DMSO-d₆) δ ppm 8.40 (s, 1H), 7.95 (d, J=7.5 Hz, 1H), 7.92-7.84 (m, 2H), 7.50 (quin, J=4.2 Hz, 1H), 7.47-7.36 (m, 2H), 7.19-7.10 (m, 2H), 6.98 (d, J=8.0 Hz, 1H), 6.85 (t, J=8.3 Hz, 1H), 5.21 (s, 2H), 3.93 (s, 2H), 3.48 (s, 2H). HRMS calcd. for C₂₁H₂₀N₂O₃ (M+H)⁺ 349.1552. found 349.1545.

### Example 161

### (R)-2-(2-((3′-(1-Aminoethyl)-[1,1′-biphenyl]-3-yl)methoxy)phenyl)acetic acid

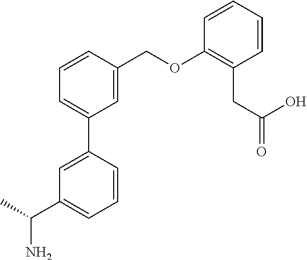 [SMILES: C[C@@H](N)c1cccc(-c2cccc(COc3ccccc3CC(=O)O)c2)c1]

Methyl 2-(2-((3-bromobenzyl)oxy)phenyl)acetate (Example 101-A) (124 mg, 0.370 mmol), (R)-tert-butyl (1-(3-(4,4,5,5-tetramethyl-1,3,2-dioxaborolan-2-yl)phenyl)ethyl)carbamate (Intermediate 26) (135 mg, 0.388 mmol), and PdCl₂(dppf).CH₂Cl₂ (15.11 mg, 0.018 mmol) were added to a microwave vial. Acetonitrile (1.5 mL), 2M aqueous K₃PO₄ (0.555 mL, 1.110 mmol) and water (0.300 mL) were added, the vial head space was flushed with nitrogen and the suspension was heated in a microwave for 60 min at 110° C. At this point, 2 mL of 2M aq. LiOH solution were added and the reaction was heated at 60° C. for 2 hours. The mixture was acidified with 2N HCl, extracted with ethyl acetate, dried over sodium sulfate, filtered, and concentrated. The crude product was dissolved in 1:1 TFA/DCM (2 mL) and stirred at room temperature for two hours. The reaction was concentrated and purified by preparative HPLC (Method B) to provide the title compound. ¹H NMR (400 MHz, DMSO-d₆) δ ppm 8.36 (s, 1H), 8.12-8.09 (m, 1H), 7.69-7.62 (m, 2H), 7.47-7.35 (m, 3H), 7.33-7.26 (m, 1H), 7.13-7.06 (m, 2H), 6.93 (d, J=8.5 Hz, 1H), 6.81 (t, J=1.0 Hz, 1H), 5.21-5.17 (m, 2H), 4.32-4.24 (m, 1H), 3.45-3.35 (m, 2H), 1.52 (d, J=6.7 Hz, 3H). HRMS calcd. for C₂₃H₂₃NO₃ (M+H)⁺ 362.1756. found 362.1756.

### Example 162

### (±)-2-(2-((6-(3-(Aminomethyl)phenyl)-2,3-dihydro-1H-inden-1-yl)oxy)phenyl)acetic acid

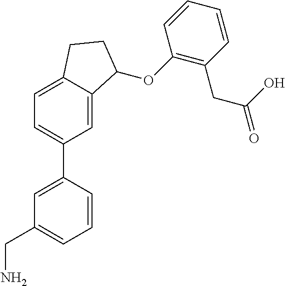 [SMILES: NCc1cccc(-c2ccc3c(c2)C(Oc2ccccc2CC(=O)O)CC3)c1]

S-Phos palladacycle (14.43 mg, 0.021 mmol), (3-(aminomethyl)phenyl)boronic acid hydrochloride (CAS #146285-80-5) (97 mg, 0.515 mmol), (±)-methyl 2-(2-((6-bromo-2,3-dihydro-1H-inden-1-yl)oxy)phenyl)acetate (Intermediate 84-B) (155 mg, 0.429 mmol) were charged in a microwave vial. Acetonitrile (1.5 mL), 2M aq K₃PO₄ (0.536 mL, 1.073 mmol) and water (0.300 mL) were added, the reaction head space was flushed with nitrogen and the suspension was heated in the microwave for 60 min at 110° C. 2 mL of a 2M aq. LiOH solution were added and the mixture was heated at 60° C. for 2 hours. The reaction mixture was acidified with 2N HCl, extracted with ethyl acetate, dried over sodium sulfate, filtered, and concentrated. The crude product was purified by preparative HPLC (Method B) to provide the title compound. ¹H NMR (400 MHz, DMSO-d₆) δ ppm 8.37 (s, 1H), 8.01 (s, 1H), 7.64 (t, J=8.0 Hz, 2H), 7.43-7.33 (m, 2H), 7.28 (d, J=7.3 Hz, 1H), 7.18 (t, J=8.1 Hz, 1H), 7.13-7.06 (m, J=8.6, 8.6 Hz, 2H), 6.87 (t, J=7.5 Hz, 1H), 5.67 (t, J=7.3 Hz, 1H), 4.04 (d, J=13.9 Hz, 1H), 3.89 (d, J=13.8 Hz, 1H), 3.62 (d, J=16.0 Hz, 1H), 3.10 (d, J=15.8 Hz, 1H), 3.06-2.86 (m, 2H), 2.10-1.92 (m, 2H). HRMS calcd. for C₂₄H₂₃NO₃ (M+H)⁺ 374.1756. found 374.1747.

### Example 163

### (±)-2-(2-(1-(3′-(aminomethyl)-[1,1′-biphenyl]-3-yl)ethoxy)phenyl)acetic acid

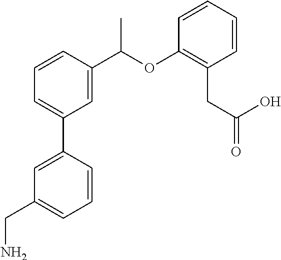 [SMILES: CC(Oc1ccccc1CC(=O)O)c1cccc(-c2cccc(CN)c2)c1]

The title compound was prepared from (±)-methyl 2-(2-(1-(3-chlorophenyl)ethoxy)phenyl)acetate (Intermediate 84-C) and (3-(aminomethyl)phenyl)boronic acid hydrochloride (CAS #146285-80-5) using the method described in Example 162. ¹H NMR (400 MHz, DMSO-d₆) δ ppm 8.15 (s, 1H), 7.91-7.88 (m, 1H), 7.62 (d, J=7.8 Hz, 1H), 7.58 (s, 1H), 7.44-7.39 (m, 2H), 7.37 (d, J=8.0 Hz, 1H), 7.26 (d, J=7.6 Hz, 1H), 7.10 (dd, J=1.7, 7.4 Hz, 1H), 6.97-6.90 (m, 1H), 6.78-6.68 (m, 2H), 5.63 (q, J=6.0 Hz, 1H), 3.95 (s, 2H), 3.79 (d, J=14.3 Hz, 1H), 3.09 (d, J=14.4 Hz, 1H), 1.55 (d, J=6.3 Hz, 3H). HRMS calcd. for C₂₃H₂₃NO₃ (M+H)⁺ 362.1756. found 362.1745.

### Example 164

### 2-(2-((4-(3-(Aminomethyl)phenyl)pyridin-2-yl)methoxy)phenyl)acetic acid

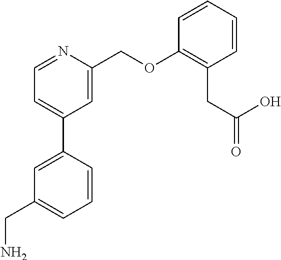 [SMILES: NCc1cccc(-c2ccnc(COc3ccccc3CC(=O)O)c2)c1]

S-Phos palladacycle (17.40 mg, 0.026 mmol), methyl 2-(2-((4-chloropyridin-2-yl)methoxy)phenyl)acetate (Intermediate 84-D) (151 mg, 0.518 mmol), (3-(aminomethyl)phenyl)boronic acid hydrochloride (CAS #146285-80-5) (179 mg, 0.621 mmol) were charged in a microwave vial. Acetonitrile (2 mL) and 2M aq sodium hydroxide (0.647 mL, 1.294 mmol) were added, the reaction head space was flushed with nitrogen and the suspension was heated in the microwave for 60 min at 110° C. The reaction was acidified with 2N HCl, extracted with ethyl acetate, dried over sodium sulfate, filtered, and concentrated. The crude product was dissolved in 1:1 TFA/DCM (2 mL) and stirred at room temperature for one hour. The reaction was concentrated and purified by preparative HPLC (Method B) to provide the title compound. ¹H NMR (400 MHz, DMSO-d₆) δ ppm 8.59 (d, J=5.2 Hz, 1H), 8.40 (s, 1H), 8.11 (s, 1H), 7.82 (d, J=7.6 Hz, 1H), 7.70 (d, J=5.1 Hz, 1H), 7.47 (t, J=9.0 Hz, 1H), 7.42-7.38 (m, 1H), 7.15-7.05 (m, 2H), 6.91 (d, J=8.1 Hz, 1H), 6.82 (t, J=7.3 Hz, 1H), 5.27 (s, 2H), 4.00 (s, 2H), 3.44 (s, 2H). HRMS calcd. for C₂₁H₂₀N₂O₃ (M+H)⁺ 349.1552. found 349.1530.

### Example 165

### 2-(2-((3′-(Aminomethyl)-4-(trifluoromethyl)-[1,1′-biphenyl]-3-yl)methoxy)phenyl)acetic acid

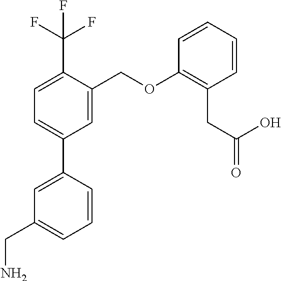 [SMILES: NCc1cccc(-c2ccc(C(F)(F)F)c(COc3ccccc3CC(=O)O)c2)c1]

S-Phos palladacycle (16.12 mg, 0.024 mmol), (3-(aminomethyl)phenyl)boronic acid hydrochloride (CAS #146285-80-5) (108 mg, 0.575 mmol), methyl 2-(2-((5-chloro-2-(trifluoromethyl)benzyl)oxy)phenyl)acetate (Intermediate 85) (172 mg, 0.479 mmol) were charged in a microwave vial. Acetonitrile (1.5 mL), 2M aq. solution of K₃PO₄ (0.599 mL, 1.199 mmol) and water (0.300 mL) were added, the reaction head space was flushed with nitrogen and the suspension was heated in the microwave for 60 min at 110° C. THF (1 mL) and 2N NaOH (2 mL) were added and the mixture was heated at 55° C. for 5 hours then stirred at room temperature overnight. The mixture was neutralized with 1N HCl, extracted with ethyl acetate, dried with sodium sulfate, filtered, and concentrated. The crude product was purified by preparative HPLC (Method B) to provide the title compound. ¹H NMR (400 MHz, DMSO-d₆) ppm 8.35 (s, 1H), 8.29 (s, 1H), 7.93-7.71 (m, 3H), 7.50-7.32 (m, 2H), 7.17-7.05 (m, 2H), 6.88-6.76 (m, 2H), 5.35 (s, 2H), 3.98 (s, 2H), 3.42 (s, 2H). HRMS calcd. for C₂₃H₂₀F₃NO₃ (M+H)⁺ 416.1474. found 416.1460.

### Example 166

### 2-(2-((3′-(Aminomethyl)-4-fluoro-[1,1′-biphenyl]-3-yl)methoxy)phenyl)acetic acid

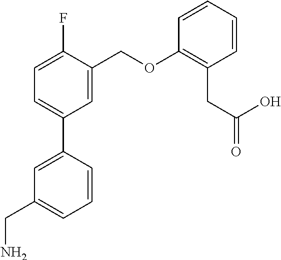 [SMILES: NCc1cccc(-c2ccc(F)c(COc3ccccc3CC(=O)O)c2)c1]

Methyl 2-(2-((5-bromo-2-fluorobenzyl)oxy)phenyl)acetate (Intermediate 84-E) (100 mg, 0.283 mmol), (3-(aminomethyl)phenyl)boronic acid hydrochloride (CAS #146285-80-5) (42.7 mg, 0.283 mmol), PdCl₂(dppf).CH₂Cl₂ adduct (CAS #95464-05-4) (9.25 mg, 0.011 mmol) were charged in a microwave vial. Acetonitrile (1.0 mL), 2M aq. K₃PO₄ (0.425 mL, 0.849 mmol) and water (0.200 mL) were added, the reaction head space was flushed with nitrogen and the suspension was heated in the microwave for 60 min at 110° C. 4 mL of 2M NaOH were added and the mixture was heated at 50° C. for 4 hours. The reaction was neutralized with 1N HCl, extracted with ethyl acetate, dried over sodium sulfate, filtered, and concentrated. The product was purified by preparative HPLC (Method B) to provide the title compound. ¹H NMR (400 MHz, DMSO-d₆) δ ppm 8.21 (s, 1H), 8.10 (dd, J=2.1, 7.0 Hz, 1H), 7.73-7.68 (m, J=4.7 Hz, 1H), 7.66 (d, J=7.8 Hz, 1H), 7.40 (t, J=7.6 Hz, 1H), 7.34-7.26 (m, 2H), 7.13-7.06 (m, 2H), 6.92 (d, J=8.2 Hz, 1H), 6.82 (t, J=7.3 Hz, 1H), 5.29 (s, 2H), 3.97 (s, 2H), 3.40 (s, 2H). HRMS calcd. for C₂₂H₂₀FNO₃ (M+H)⁺ 366.1505. found 366.1502.

### Example 167

### (S)-2-(2-((3′-(1-Amino-2-hydroxyethyl)-4-(trifluoromethyl)-[1,1′-biphenyl]-3-yl)methoxy)phenyl)acetic acid

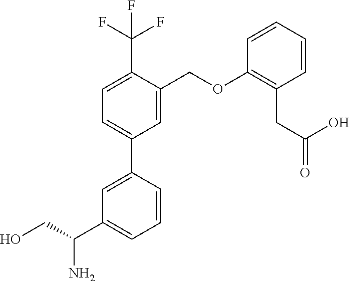 [SMILES: N[C@H](CO)c1cccc(-c2ccc(C(F)(F)F)c(COc3ccccc3CC(=O)O)c2)c1]

S-Phos palladacycle (9.37 mg, 0.014 mmol), (S)-tert-butyl (2-hydroxy-1-(3-(4,4,5,5-tetramethyl-1,3,2-dioxaborolan-2-yl)phenyl)ethyl)carbamate (Intermediate 34-B) (124 mg, 0.307 mmol), methyl 2-(2-((5-chloro-2-(trifluoromethyl)benzyl)oxy)phenyl)acetate (Intermediate 85) (100 mg, 0.279 mmol) were charged in a microwave vial. Acetonitrile (1.0 mL), 2M aq. K₃PO₄ (0.348 mL, 0.697 mmol) and water (0.200 mL) were added, the reaction head space was flushed with nitrogen and the suspension was heated in the microwave for 60 min at 110° C. THF (2 mL) and 2N NaOH (3 mL) were added and the mixture was heated at 50° C. overnight. The reaction was neutralized with 1N HCl, extracted with ethyl acetate, dried over sodium sulfate, filtered, and concentrated. The crude product was dissolved in 1:1 TFA/DCM (2 mL) and stirred at room temperature for two hours. The reaction mixture was concentrated and purified by preparative HPLC (Method B) to provide the title compound. ¹H NMR (400 MHz, DMSO-d₆) δ ppm 8.34 (br. s., 2H), 7.92 (d, J=8.0 Hz, 1H), 7.84 (d, J=8.0 Hz, 1H), 7.79 (d, J=8.0 Hz, 1H), 7.46 (t, J=7.6 Hz, 1H), 7.38 (d, J=7.5 Hz, 1H), 7.16-7.08 (m, 2H), 6.85 (s, 2H), 5.34 (s, 2H), 4.20 (t, J=5.7 Hz, 1H), 3.79-3.69 (m, 2H), 3.48 (d, J=15.5 Hz, 1H), 3.38 (d, J=15.0 Hz, 1H). HRMS calcd. for C₂₄H₂₂F₃NO₄ (M+H)⁺ 446.1579. found 446.1566.

### Example 168

### 2-(2-((3′-(Aminomethyl)-[1,1′-biphenyl]-3-yl)methoxy)-5-bromophenyl)acetic acid

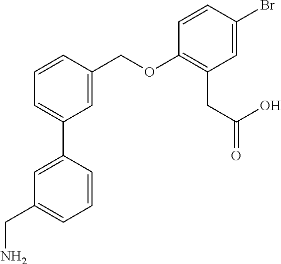 [SMILES: NCc1cccc(-c2cccc(COc3ccc(Br)cc3CC(=O)O)c2)c1]

5N aqueous NaOH solution (0.555 mL, 2.78 mmol) was added to a THF (0.5 mL) solution of methyl 2-(5-bromo-2-((3′-(((tert-butoxycarbonyl)amino)methyl)-[1,1′-biphenyl]-3-yl)methoxy)phenyl)acetate (Intermediate 84-F) (150 mg, 0.278 mmol) at rt and then heated at 50° C. overnight. The reaction was cooled, acidified to pH 2 with aq. HCl, extracted with ethyl acetate, dried over sodium sulfate, filtered, and concentrated. The crude product was dissolved in 1:1 TFA/DCM (2 mL) and stirred at room temperature for two hours. The reaction mixture was concentrated and purified by preparative HPLC (Method B) to provide the title compound. ¹H NMR (400 MHz, DMSO-d₆) δ ppm 8.25 (s, 1H), 7.97 (s, 1H), 7.66 (t, J=9.2 Hz, 2H), 7.46-7.43 (m, 1H), 7.42 (d, J=4.8 Hz, 1H), 7.37 (t, J=7.6 Hz, 1H), 7.30 (d, J=7.7 Hz, 1H), 7.27 (d, J=2.6 Hz, 1H), 7.23 (dd, J=2.6, 8.7 Hz, 1H), 6.88 (d, J=8.8 Hz, 1H), 5.23 (s, 2H), 3.99 (s, 2H), 3.39 (s, 2H). HRMS calcd. for C₂₂H₂₀BrNO₃ (M+H)⁺ 426.0705 and 428.0685. found 426.0700 and 428.0683.

### Example 169

### (S)-2-(2-((3′-(1-Amino-2-methoxyethyl)-[1,1′-biphenyl]-3-yl)methoxy)phenyl)acetic acid

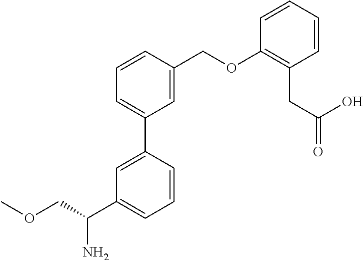 [SMILES: COC[C@@H](N)c1cccc(-c2cccc(COc3ccccc3CC(=O)O)c2)c1]

The title compound was prepared from (S)-tert-butyl (1-(3-bromophenyl)-2-methoxyethyl)carbamate (Intermediate 36) and methyl 2-(2-((3-(4,4,5,5-tetramethyl-1,3,2-dioxaborolan-2-yl)benzyl)oxy)phenyl)acetate (Example 101-B) using the method described in Example 167, using PdCl₂(dppf).CH₂Cl₂ adduct instead of S-Phos palladacycle. ¹H NMR (400 MHz, DMSO-d₆) δ ppm 8.20 (s, 1H), 8.00 (s, 1H), 7.66 (t, J=8.0 Hz, 2H), 7.48-7.37 (m, 3H), 7.34 (d, J=7.6 Hz, 1H), 7.17-7.11 (m, 2H), 6.98 (d, J=7.8 Hz, 1H), 6.84 (dt, J=0.9, 7.3 Hz, 1H), 5.17 (s, 2H), 4.26 (dd, J=5.1, 8.4 Hz, 1H), 3.78 (dd, J=8.6, 9.6 Hz, 1H), 3.55-3.48 (m, 1H), 3.44 (d, J=8.2 Hz, 2H), 3.29 (s, 3H). HRMS calcd. for C₂₄H₂₅NO₄ (M+H)⁺ 392.1862. found 392.1858.

### Example 170

### (S)-2-(2-((4-(3-(1-Amino-2-hydroxyethyl)phenyl)pyridin-2-yl)methoxy)phenyl)acetic acid

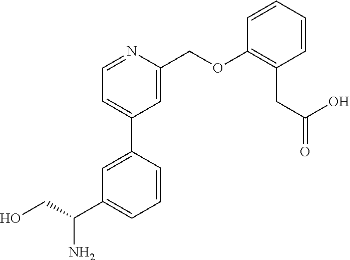 [SMILES: N[C@H](CO)c1cccc(-c2ccnc(COc3ccccc3CC(=O)O)c2)c1]

The title compound was prepared from methyl 2-(2-((4-chloropyridin-2-yl)methoxy)phenyl)acetate (Intermediate 84-D) and (S)-tert-butyl (2-hydroxy-1-(3-(4,4,5,5-tetramethyl-1,3,2-dioxaborolan-2-yl)phenyl)ethyl)carbamate (Intermediate 34-B) using the method described in Example 167. ¹H NMR (400 MHz, DMSO-d₆) δ ppm 8.59 (d, J=5.3 Hz, 1H), 8.42 (s, 1H), 8.09 (s, 1H), 7.85 (d, J=7.8 Hz, 1H), 7.72 (dd, J=1.6, 5.3 Hz, 1H), 7.47 (t, J=7.7 Hz, 1H), 7.42 (d, J=7.8 Hz, 1H), 7.16-7.07 (m, 2H), 6.93 (d, J=7.6 Hz, 1H), 6.83 (dt, J=0.9, 7.4 Hz, 1H), 5.27 (s, 2H), 4.20 (dd, J=4.7, 6.8 Hz, 1H), 3.78-3.68 (m, 2H), 3.55-3.35 (m, 2H). HRMS calcd. for C₂₂H₂₂N₂O₄ (M+H)⁺ 379.1658. found 379.1653.

### Example 171

### (±)-2-(2-((3′-(2-Hydroxy-1-(methylamino)ethyl)-[1,1′-biphenyl]-3-yl)methoxy)phenyl)acetic acid

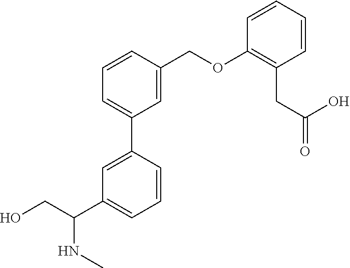 [SMILES: CNC(CO)c1cccc(-c2cccc(COc3ccccc3CC(=O)O)c2)c1]

The title compound was prepared from methyl 2-(2-((3-(4,4,5,5-tetramethyl-1,3,2-dioxaborolan-2-yl)benzyl)oxy)phenyl)acetate (Example 101-B) and (±)-2-(3-bromophenyl)-2-(methylamino)ethanol (CAS #1184796-75-5) using the method described in Example 166, except that DMF was used instead of acetonitrile as solvent. ¹H NMR (400 MHz, DMSO-d₆) ppm 7.95 (d, J=4.4 Hz, 2H), 7.66-7.55 (m, 2H), 7.48-7.34 (m, 2H), 7.33-7.24 (m, 2H), 7.16-7.04 (m, 2H), 6.95 (d, J=7.7 Hz, 1H), 6.81 (dt, J=0.9, 7.3 Hz, 1H), 5.15 (s, 2H), 3.79-3.68 (m, 1H), 3.66-3.58 (m, 1H), 3.58-3.47 (m, 2H), 3.45-3.37 (m, 2H), 3.34 (d, J=8.0 Hz, 3H). HRMS calcd. for C₂₄H₂₅NO₄ (M+H)⁺ 392.1862. found 392.1862.

### Example 172

### (±)-2-(2-((6-(3-((S)-1-Amino-2-hydroxyethyl)phenyl)-2,3-dihydro-1H-inden-1-yl)oxy)phenyl)acetic acid

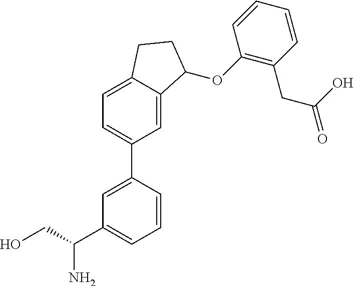 [SMILES: N[C@H](CO)c1cccc(-c2ccc3c(c2)C(Oc2ccccc2CC(=O)O)CC3)c1]

The title compound was prepared from (±)-methyl 2-(2-((6-bromo-2,3-dihydro-1H-inden-1-yl)oxy)phenyl)acetate (Intermediate 84-B) and (S)-tert-Butyl (2-hydroxy-1-(3-(4,4,5,5-tetramethyl-1,3,2-dioxaborolan-2-yl)phenyl)ethyl)carbamate (Intermediate 34-B) using the method described in Example 169. ¹H NMR (400 MHz, DMSO-d₆)=7.79-7.69 (m, 1H), 7.58-7.53 (m, 1H), 7.45-7.34 (m, 4H), 7.31 (d, J=7.7 Hz, 1H), 7.26-7.15 (m, 1H), 6.96 (dd, J=2.1, 17.2 Hz, 1H), 6.91-6.84 (m, 1H), 6.66 (dd, J=2.3, 8.1 Hz, 1H), 4.28-4.14 (m, 2H), 3.68-3.56 (m, 2H), 3.44-3.31 (m, 1H), 3.29-3.13 (m, 1H), 3.05-2.85 (m, 2H), 2.05-1.84 (m, 2H) HRMS calcd. for C₂₅H₂₅NO₄ (M+H)⁺ 404.1875. found 404.1858.

### Example 173

### (S)-2-(2-((3′-(1-Amino-2-ethoxyethyl)-[1,1′-biphenyl]-3-yl)methoxy)phenyl)acetic acid

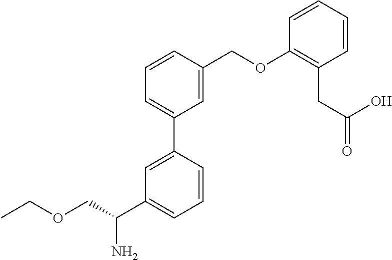 [SMILES: CCOC[C@@H](N)c1cccc(-c2cccc(COc3ccccc3CC(=O)O)c2)c1]

The title compound was prepared from (S)-tert-butyl (1-(3-bromophenyl)-2-ethoxyethyl)carbamate (Intermediate 37) and methyl 2-(2-((3-(4,4,5,5-tetramethyl-1,3,2-dioxaborolan-2-yl)benzyl)oxy)phenyl)acetate (Example 101-B) using the method described in Example 169, except that DMF was used instead of acetonitrile as solvent. ¹H NMR (400 MHz, DMSO-d₆) δ ppm 8.20 (s, 1H), 8.00 (s, 1H), 7.66 (t, J=7.6 Hz, 2H), 7.47-7.39 (m, 3H), 7.38-7.33 (m, 1H), 7.17-7.11 (m, 2H), 6.98 (d, J=8.0 Hz, 1H), 6.84 (dt, J=0.9, 7.3 Hz, 1H), 5.17 (s, 2H), 4.24 (dd, J=5.0, 8.5 Hz, 1H), 3.79 (dd, J=8.7, 9.7 Hz, 2H), 3.56 (dd, J=5.0, 9.9 Hz, 1H), 3.49 (dq, J=1.1, 7.0 Hz, 2H), 3.43 (d, J=8.1 Hz, 1H), 1.12 (t, J=7.0 Hz, 3H). HRMS calcd. for C₂₅H₂₇NO₄ (M+H)⁺ 406.2018. found 406.2015.

### Example 174

### 2-(2-((3′-(Aminomethyl)-[1,1′-biphenyl]-3-yl)methoxy)-3-bromophenyl)acetic acid

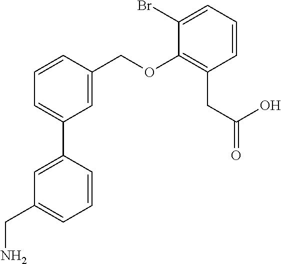 [SMILES: NCc1cccc(-c2cccc(COc3c(Br)cccc3CC(=O)O)c2)c1]

5N aqueous solution of NaOH (0.756 mL, 3.78 mmol) was added to a THF (2.0 mL) solution of methyl 2-(3-bromo-2-((3′-(((tert-butoxycarbonyl)amino)methyl)-[1,1′-biphenyl]-3-yl)methoxy)phenyl)acetate (Intermediate 84-G) (408 mg, 0.756 mmol) at rt and then heated at 50° C. overnight. The reaction was cooled, acidified to pH 2 with aq. HCl, extracted with ethyl acetate, dried over sodium sulfate, filtered, and concentrated. The crude product was dissolved in 1:1 TFA/DCM (2 mL) and stirred at room temperature for three hours. The mixture was concentrated and purified by preparative HPLC (Method B), lyophilized, and the resulting material was washed with methanol and filtered to provide the title compound. ¹H NMR (400 MHz, DMSO-d₆) δ ppm 8.27 (d, J=9.2 Hz, 2H), 7.72 (t, J=8.5 Hz, 2H), 7.51-7.40 (m, 3H), 7.35 (dd, J=7.8, 11.3 Hz, 2H), 7.22 (dd, J=1.6, 7.5 Hz, 1H), 7.02 (t, J=7.7 Hz, 1H), 5.06 (s, 2H), 4.01 (s, 2H), 3.49 (s, 2H). HRMS calcd. for C₂₂H₂₀BrNO₃ (M+H)⁺ 426.0705 and 428.0685. found 426.0687 and 428.0668.

### Example 175

### a) (±)-(2-((7-(3-(Aminomethyl)phenyl)-1,2,3,4-tetrahydronaphthalen-1-yl)oxy)phenyl)acetic acid

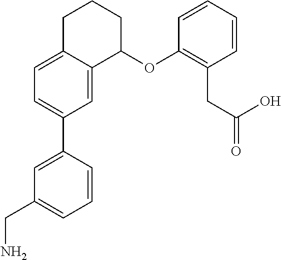 [SMILES: NCc1cccc(-c2ccc3c(c2)C(Oc2ccccc2CC(=O)O)CCC3)c1]

The title compound was prepared from methyl 2-(2-((7-bromo-1,2,3,4-tetrahydronaphthalen-1-yl)oxy)phenyl)acetate (Intermediate 84-H) and (3-(aminomethyl)phenyl)boronic acid hydrochloride (CAS #146285-80-5) using the method described in Example 165, except that DMF was used instead of acetonitrile as solvent. ¹H NMR (400 MHz, DMSO-d₆) δ ppm 8.43-8.37 (m, 1H), 7.95 (d, J=1.6 Hz, 1H), 7.64 (d, J=7.8 Hz, 1H), 7.56 (dd, J=1.8, 8.0 Hz, 1H), 7.38 (t, J=7.6 Hz, 1H), 7.26 (d, J=7.6 Hz, 1H), 7.21 (d, J=8.1 Hz, 1H), 7.18-7.11 (m, J=1.8 Hz, 1H), 7.08 (s, 2H), 6.82 (dt, J=1.0, 7.3 Hz, 1H), 5.45 (dd, J=5.4, 8.6 Hz, 1H), 4.02 (d, J=13.1 Hz, 1H), 3.92 (d, J=13.0 Hz, 1H), 3.59 (d, J=15.5 Hz, 1H), 3.06 (d, J=15.7 Hz, 1H), 2.86-2.79 (m, 2H), 2.47-2.40 (m, 1H), 1.99-1.86 (m, 2H), 1.80-1.65 (m, 1H). HRMS calcd. for C₂₅H₂₅NO₃ (M+H)⁺ 388.1913. found 388.1904.

### b) (+) or (−)-2-((7-(3-(Aminomethyl)phenyl)-1,2,3,4-tetrahydronaphthalen-1-yl)oxy)phenyl)acetic acid

Resolution of the enantiomers of (2-((7-(3-(aminomethyl)phenyl)-1,2,3,4-tetrahydronaphthalen-1-yl)oxy)phenyl)acetic acid was achieved by chiral SFC using a CHIRALPAK® AD-H column with 30% IPA+5 mM NH₄OH in CO₂ to give (+) or (−)-(2-((7-(3-(aminomethyl)phenyl)-1,2,3,4-tetrahydronaphthalen-1-yl)oxy)phenyl)acetic acid (tᵣ=4.1 min) and (−) or (+)-(2-((7-(3-(aminomethyl)phenyl)-1,2,3,4-tetrahydronaphthalen-1-yl)oxy)phenyl)acetic acid (tᵣ=9.5 min).

### Example 176

### 2-(2-((4-(5-(Aminomethyl)-2-fluorophenyl)pyridin-2-yl)methoxy)phenyl)acetic acid

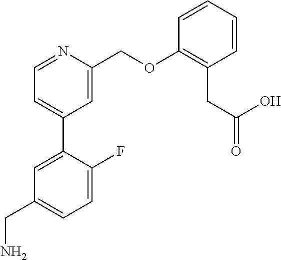 [SMILES: NCc1ccc(F)c(-c2ccnc(COc3ccccc3CC(=O)O)c2)c1]

The title compound was prepared from methyl 2-(2-((4-chloropyridin-2-yl)methoxy)phenyl)acetate (Intermediate 84-D) and (5-(aminomethyl)-2-fluorophenyl)boronic acid hydrochloride (CAS #1072946-46-3) using the method described in Example 166, except that DMF was used instead of Acetonitrile as solvent. ¹H NMR (400 MHz, DMSO-d₆) δ ppm 8.61 (d, J=5.3 Hz, 1H), 8.21 (dd, J=2.0, 7.7 Hz, 1H), 7.96 (s, 1H), 7.68-7.58 (m, 1H), 7.45 (br. s., 1H), 7.33 (dd, J=8.4, 11.3 Hz, 1H), 7.15-7.05 (m, 2H), 6.93 (d, J=8.0 Hz, 1H), 6.82 (t, J=6.9 Hz, 1H), 5.29 (s, 2H), 3.97 (s, 2H), 3.41 (s, 2H). HRMS calcd. for C₂₁H₁₉FN₂O₃(M+H)⁺ 367.1458. found 367.1457.

### Example 177

### a) (±)-2-(2-((6-(3-(Aminomethyl)phenyl)chroman-4-yl)oxy)phenyl)acetic acid

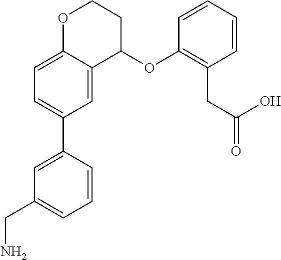 [SMILES: NCc1cccc(-c2ccc3c(c2)C(Oc2ccccc2CC(=O)O)CCO3)c1]

The title compound was prepared from methyl 2-(2-((6-chlorochroman-4-yl)oxy)phenyl)acetate (Intermediate 84-1) and (3-(aminomethyl)phenyl)boronic acid hydrochloride (CAS #146285-80-5) using the method described in Example 165, except that DMF was used instead of Acetonitrile as solvent. ¹H NMR (400 MHz, DMSO-d₆) δ ppm 8.36 (s, 1H), 7.94 (d, J=2.0 Hz, 1H), 7.60 (d, J=7.1 Hz, 1H), 7.57 (dd, J=2.3, 8.5 Hz, 1H), 7.36 (t, J=7.6 Hz, 1H), 7.23 (d, J=8.2 Hz, 1H), 7.19-7.13 (m, 1H), 7.13-7.06 (m, 2H), 6.91-6.83 (m, 2H), 5.54 (dd, J=5.4, 8.0 Hz, 1H), 4.41-4.28 (m, 2H), 4.01 (d, J=13.0 Hz, 1H), 3.92 (d, J=13.3 Hz, 1H), 3.58 (d, J=15.5 Hz, 1H), 3.08 (d, J=15.8 Hz, 1H), 2.08-1.95 (m, 2H). HRMS calcd. for C₂₄H₂₃NO₄ (M+H)⁺ 390.1705. found 390.1697.

### b) (+) or (−)-2-(2-((6-(3-(aminomethyl)phenyl)chroman-4-yl)oxy)phenyl)acetic acid

Resolution of the enantiomers of 2-(2-((6-(3-(aminomethyl)phenyl)chroman-4-yl)oxy)phenyl)acetic acid was achieved by chiral SFC using a CHIRALPAK® AD-H column with 45% IPA+5 mM NH₄OH in CO₂ to give (+) or (−)-2-(2-((6-(3-(aminomethyl)phenyl)chroman-4-yl)oxy)phenyl)acetic acid (tᵣ=3.2 min) and (−) or (+)-2-(2-((6-(3-(aminomethyl)phenyl)chroman-4-yl)oxy)phenyl)acetic acid (tᵣ=8.8 min).

### Example 178

### 2-(2-((6-(3-(Aminomethyl)phenyl)-1-tosyl-1H-indazol-4-yl)methoxy)phenyl)acetic acid

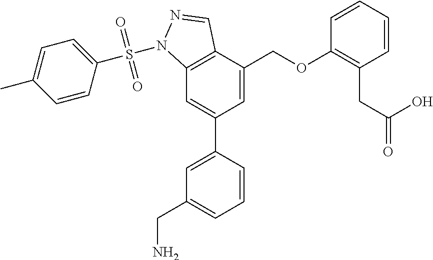 [SMILES: Cc1ccc(S(=O)(=O)n2ncc3c(COc4ccccc4CC(=O)O)cc(-c4cccc(CN)c4)cc32)cc1]

A suspension of 2M aqueous K₃PO₄ (0.254 mL, 0.507 mmol), (3-(aminomethyl)phenyl)boronic acid hydrochloride (CAS #146285-80-5) (80 mg, 0.317 mmol), tert-butyl 2-(2-((6-bromo-1-tosyl-1H-indazol-4-yl)methoxy)phenyl)acetate (Intermediate 84-J) (145 mg, 0.254 mmol), and PdCl₂(dppf).CH₂Cl₂ adduct (CAS #95464-05-4) (9.28 mg, 0.013 mmol) in DMF (2.0 mL) was heated for 1 hour in the microwave at 110° C. The reaction was filtered, diluted with water, extracted with ethyl acetate, dried over sodium sulfate, filtered, and concentrated. The crude was dissolved in 1:1 TFA/DCM (2 mL) and stirred at room temperature for two hours. The mixture was then concentrated and purified by preparative HPLC (Method B) to provide the title compound. ¹H NMR (400 MHz, DMSO-d₆) δ ppm 8.77 (d, J=0.8 Hz, 1H), 8.28 (s, 2H), 8.07 (s, 1H), 7.89 (d, J=8.3 Hz, 2H), 7.83 (d, J=8.1 Hz, 1H), 7.52 (t, J=7.6 Hz, 1H), 7.45-7.36 (m, 3H), 7.15-7.03 (m, 2H), 6.94 (d, J=7.7 Hz, 1H), 6.81 (dt, J=0.9, 7.4 Hz, 1H), 5.56 (s, 2H), 4.00 (s, 2H), 3.41 (s, 2H), 2.33 (s, 3H). HRMS calcd. for C₃₀H₂₇N₃O₅S (M+H) 542.1927. found 542.1963.

### Example 179

### 2-(2-((6-(3-(Aminomethyl)phenyl)-1H-indazol-4-yl)methoxy)phenyl)acetic acid

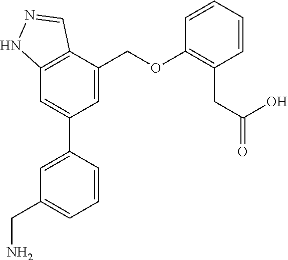 [SMILES: NCc1cccc(-c2cc(COc3ccccc3CC(=O)O)c3cn[nH]c3c2)c1]

The title compound was prepared from tert-butyl 2-(2-((6-bromo-1-tosyl-1H-indazol-4-yl)methoxy)phenyl)acetate (Intermediate 84-J) and (3-(aminomethyl)phenyl)boronic acid hydrochloride (CAS #146285-80-5) using the method described in Example 169, except that DMF was used instead of acetonitrile as solvent. ¹H NMR (400 MHz, DMSO-d₆) δ ppm 8.29 (s, 1H), 8.22 (s, 1H), 7.82 (s, 1H), 7.76 (d, J=7.3 Hz, 1H), 7.73 (s, 1H), 7.44 (t, J=7.8 Hz, 1H), 7.31 (d, J=7.2 Hz, 1H), 7.16-7.04 (m, 2H), 6.97 (d, J=8.0 Hz, 1H), 6.81 (t, J=7.3 Hz, 1H), 5.56 (s, 2H), 3.99 (s, 2H), 3.45 (s, 2H). HRMS calcd. for C₂₃H₂₁N₃O₃ (M+H)⁺ 388.1661. found 388.1726.

### Example 180

### (R)-2-(2-((4-(3-(1-Aminoethyl)phenyl)pyridin-2-yl)methoxy)phenyl)acetic acid

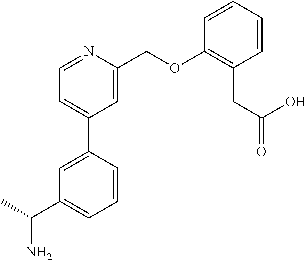 [SMILES: C[C@@H](N)c1cccc(-c2ccnc(COc3ccccc3CC(=O)O)c2)c1]

A suspension of (R)-tert-butyl (1-(3-(4,4,5,5-tetramethyl-1,3,2-dioxaborolan-2-yl)phenyl)ethyl)carbamate (Intermediate 26) (148 mg, 0.426 mmol), methyl 2-(2-((4-chloropyridin-2-yl)methoxy)phenyl)acetate (Intermediate 84-D) (113 mg, 0.387 mmol), 2M aqueous K₃PO₄ (0.581 mL, 1.162 mmol) and S-Phos palladacycle (13.0 mg, 0.019 mmol) in DMF (2 mL) was heated in the microwave for 1.5 hours at 110° C. 5N aqueous NaOH (0.387 mL, 1.94 mmol) was added and the reaction was heated at 50° C. overnight. The mixture was diluted with water, extracted with ethyl acetate, dried over sodium sulfate, filtered, and concentrated. The crude was dissolved in 1:1 TFA/DCM (2 mL) and stirred at room temperature for two hours. The mixture was concentrated and purified by preparative HPLC (Method B) to provide the title compound. ¹H NMR (400 MHz, DMSO-d₆) δ ppm 8.59 (d, J=5.2 Hz, 1H), 8.49 (s, 1H), 8.19 (d, J=0.9 Hz, 1H), 7.83 (d, J=7.7 Hz, 1H), 7.71 (dd, J=1.8, 5.3 Hz, 1H), 7.48 (t, J=7.7 Hz, 1H), 7.41 (d, J=7.6 Hz, 1H), 7.10 (dq, J=1.7, 7.4 Hz, 2H), 6.92 (d, J=7.6 Hz, 1H), 6.83 (dt, J=0.9, 7.4 Hz, 1H), 5.31-5.19 (m, 2H), 4.31 (q, J=6.8 Hz, 1H), 3.51-3.33 (m, 2H), 1.53 (d, J=6.7 Hz, 3H). HRMS calcd. for C₂₂H₂₂N₂O₃ (M+H)⁺ 363.1709. found 363.1707.

### Example 181

### 2-(2-((7-(3-((S)-1-Amino-2-hydroxyethyl)phenyl)-1,2,3,4-tetrahydronaphthalen-1-yl)oxy)phenyl)acetic acid (mixture of diastereomers)

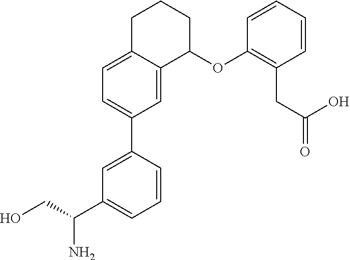 [SMILES: N[C@H](CO)c1cccc(-c2ccc3c(c2)C(Oc2ccccc2CC(=O)O)CCC3)c1]

A suspension of methyl 2-(2-((7-(4,4,5,5-tetramethyl-1,3,2-dioxaborolan-2-yl)-1,2,3,4-tetrahydronaphthalen-1-yl)oxy)phenyl)acetate (Intermediate 87) (270 mg, 0.639 mmol), (S)-2-amino-2-(3-bromophenyl)-ethan-1-ol (CAS #209963-05-3) (210 mg, 0.831 mmol), 2M aqueous K₃PO₄ (0.320 mL, 0.639 mmol) and PdCl₂(dppf).CH₂Cl₂ adduct (CAS #95464-05-4) (23.4 mg, 0.032 mmol) in DMF (3 mL) was heated in the microwave for 1 hour at 110° C. and then for 30 minutes at 140° C. Additional (S)-2-amino-2-(3-bromophenyl)-ethan-1-ol (CAS #209963-05-3) (210 mg, 0.831 mmol), 2M aqueous K₃PO₄ (0.320 mL, 0.639 mmol) and PdCl₂(dppf).CH₂Cl₂ adduct (CAS #95464-05-4) (23.4 mg, 0.032 mmol) were added and this was heated in the microwave for 1 hour at 140° C. The reaction mixture was cooled, diluted with water, extracted with ethyl acetate, dried over sodium sulfate, filtered, and concentrated. The crude was dissolved in MeOH (4 mL) and NaOH (35.4 mg, 0.885 mmol) was added and the reaction was stirred at room temperature overnight and then heated at 50° C. for 1 hour. The mixture was concentrated and purified by preparative HPLC (Method B) to provide the title compound. ¹H NMR (400 MHz, DMSO-d₆) δ ppm 8.33-8.27 (m, 1H), 8.18-8.14 (m, 1H), 8.09-8.06 (m, 1H), 8.01-7.92 (m, 1H), 7.92-7.82 (m, 2H), 7.82-7.74 (m, 1H), 7.57-7.40 (m, 3H), 7.07 (s, 10H), 6.89-6.76 (m, 2H), 5.51-5.46 (m, 1H), 5.46-5.39 (m, 1H), 5.07-4.97 (m, 1H), 4.70-4.57 (m, 1H), 4.00-3.88 (m, 2H), 3.70-3.61 (m, 1H), 3.60-3.46 (m, 2H), 3.46-3.36 (m, 2H), 3.15-3.02 (m, 2H), 2.92-2.71 (m, 4H), 2.25-2.10 (m, 3H), 2.01-1.84 (m, 4H). HRMS calcd. for C₂₆H₂₇NO₄ (M+H)⁺ 418.2018. found 418.2018.

### Example 182

### 2-(2-((4-(3-(Aminomethyl)-2-fluorophenyl)pyridin-2-yl)methoxy)phenyl)acetic acid

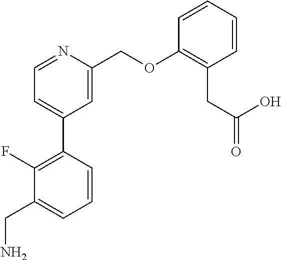 [SMILES: NCc1cccc(-c2ccnc(COc3ccccc3CC(=O)O)c2)c1F]

The title compound was prepared from methyl 2-(2-((4-chloropyridin-2-yl)methoxy)phenyl)acetate (Intermediate 84-D) and (3-(aminomethyl)-2-fluorophenyl)boronic acid hydrochloride (CAS #1072946-44-1) using the method described in Example 165, except that DMF was used instead of acetonitrile as solvent. ¹H NMR (400 MHz, DMSO-d₆ with 5 μL of TFA) δ ppm 8.76-8.67 (m, 1H), 8.40-8.14 (m, 2H), 7.82-7.75 (m, 1H), 7.70 (t, J=7.6 Hz, 1H), 7.67-7.54 (m, 2H), 7.48-7.38 (m, 1H), 7.29-7.19 (m, 2H), 7.06 (d, J=8.0 Hz, 1H), 6.93 (t, J=7.4 Hz, 1H), 5.29 (br. s., 2H), 4.17 (q, J=5.7 Hz, 2H), 3.63 (s, 2H). HRMS calcd. for C₂₁H₁₉FN₂O₃(M+H)⁺ 367.1458. found 367.1452.

### Example 183

### (±)-2-(2-(1-(3′-(Aminomethyl)-2′-fluoro-[1,1′-biphenyl]-3-yl)ethoxy)phenyl)acetic acid

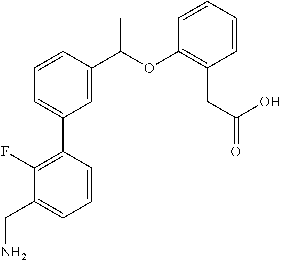 [SMILES: CC(Oc1ccccc1CC(=O)O)c1cccc(-c2cccc(CN)c2F)c1]

The title compound was prepared from (±)-methyl 2-(2-(1-(3-chlorophenyl)ethoxy)phenyl)acetate (Intermediate 84-C) and (3-(aminomethyl)-2-fluorophenyl)boronic acid hydrochloride (CAS #1072946-44-1) using the method described in Example 165, except that DMF was used instead of acetonitrile as solvent. ¹H NMR (400 MHz, METHANOL-d₄) 5 ppm=7.60 (s, 1H), 7.56 (dt, J=1.7, 7.6 Hz, 1H), 7.48-7.41 (m, 3H), 7.35-7.32 (m, 1H), 7.17 (dd, J=1.5, 7.3 Hz, 1H), 7.07 (dt, J=1.7, 7.9 Hz, 1H), 6.86-6.83 (m, 1H), 6.83-6.80 (m, 1H), 6.76 (d, J=8.3 Hz, 1H), 5.47 (q, J=6.4 Hz, 1H), 4.24 (s, 2H), 3.69-3.66 (m, J=4.2 Hz, 2H), 1.64 (d, J=6.4 Hz, 3H). HRMS calcd. for C₂₃H₂₂FNO₃ (M+H)⁺ 380.1662. found 380.1656.

### Example 184

### (S)-2-(2-((4-(3-(1-amino-2-methoxyethyl)phenyl)pyridin-2-yl)methoxy)phenyl)acetic acid

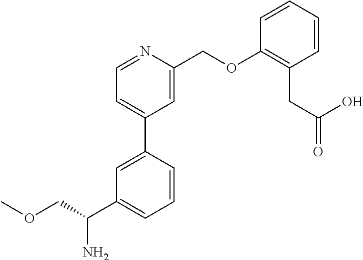 [SMILES: COC[C@@H](N)c1cccc(-c2ccnc(COc3ccccc3CC(=O)O)c2)c1]

The title compound was prepared from methyl 2-(2-((4-chloropyridin-2-yl)methoxy)phenyl)acetate (Intermediate 84-D) and (S)-tert-butyl (2-methoxy-1-(3-(4,4,5,5-tetramethyl-1,3,2-dioxaborolan-2-yl)phenyl)ethyl)carbamate (Intermediate 88) using the method described in Example 165, except that DMF was used instead of acetonitrile as solvent. ¹H NMR (400 MHz, DMSO-d₆) δ ppm 8.61-8.54 (m, 1H), 8.20-8.18 (m, 1H), 8.14-8.08 (m, 1H), 7.81-7.76 (m, 1H), 7.66-7.62 (m, 1H), 7.43-7.38 (m, 2H), 7.16-7.10 (m, 1H), 7.08-7.01 (m, 1H), 6.93-6.87 (m, 1H), 6.83-6.76 (m, 1H), 5.17 (s, 2H), 4.13-4.01 (m, 1H), 3.51-3.38 (m, 4H), 3.19 (s, 3H). HRMS calcd. for C₂₃H₂₄N₂O₄ (M+H)⁺ 393.1814. found 393.1819.

### Example 185

### (S)-2-(2-((4-(3-(1-Amino-2-hydroxyethyl)phenyl)-6-methylpyridin-2-yl)methoxy)phenyl)acetic acid

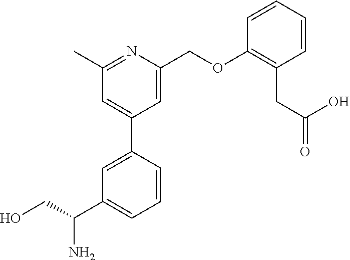 [SMILES: Cc1cc(-c2cccc([C@H](N)CO)c2)cc(COc2ccccc2CC(=O)O)n1]

A suspension of (S)-tert-butyl (2-hydroxy-1-(3-(4,4,5,5-tetramethyl-1,3,2-dioxaborolan-2-yl)phenyl)ethyl)carbamate (Intermediate 34-B) (213 mg, 0.586 mmol), tert-butyl 2-(2-((4-chloro-6-methylpyridin-2-yl)methoxy)phenyl)acetate (Intermediate 84-K) (163 mg, 0.469 mmol), 2M aqueous K₃PO₄ (0.703 mL, 1.406 mmol) and S-Phos palladacycle (15.8 mg, 0.023 mmol) in DMF (2 mL) was heated in the microwave for 1 hour at 110° C. The reaction was filtered, diluted with water, extracted with EtOAc, dried over sodium sulfate, filtered and concentrated. The crude product was dissolved in DCM (1 mL) and then 4N HCl in dioxane (4 mL) was added at room temperature, and stirred for 3 hours. Acetonitrile was added and the reaction mixture was concentrated and then purified by preparative HPLC (Method A) to provide the title compound as the TFA salt. ¹H NMR (TFA salt, 400 MHz, DMSO-d₆) δ ppm 8.40 (br. d, J=3.4 Hz, 3H), 7.94 (s, 1H), 7.85 (td, J=1.9, 6.6 Hz, 1H), 7.74 (s, 1H), 7.64-7.60 (m, 1H), 7.60-7.54 (m, 2H), 7.29-7.20 (m, 2H), 7.04 (d, J=8.1 Hz, 1H), 6.93 (dt, J=0.9, 7.4 Hz, 1H), 5.24 (s, 2H), 4.46-4.34 (m, 1H), 3.84-3.69 (m, 2H), 3.66 (s, 2H), 2.60 (s, 3H). HRMS calcd. for C₂₃H₂₄N₂O₄ (M+H)⁺ 393.1814. found 393.1807.

### Example 186

### (S)-2-(2-((6-(3-(1-Amino-2-hydroxyethyl)phenyl)-1-tosyl-1H-indazol-4-yl)methoxy)phenyl)acetic acid

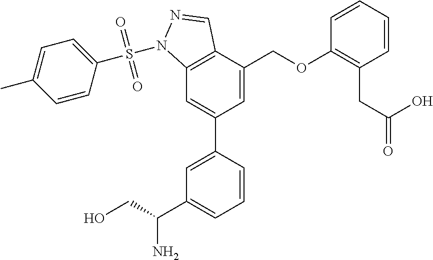 [SMILES: Cc1ccc(S(=O)(=O)n2ncc3c(COc4ccccc4CC(=O)O)cc(-c4cccc([C@H](N)CO)c4)cc32)cc1]

The title compound was prepared from tert-butyl 2-(2-((6-bromo-1-tosyl-1H-indazol-4-yl)methoxy)phenyl)acetate (Intermediate 84-J) and (S)-tert-butyl (2-hydroxy-1-(3-(4,4,5,5-tetramethyl-1,3,2-dioxaborolan-2-yl)phenyl)ethyl)carbamate (Intermediate 34-B) using the method described in Example 178, except that acetonitrile was used instead of DMF as solvent. ¹H NMR (400 MHz, DMSO-d₆) δ ppm 8.81 (d, J=0.7 Hz, 1H), 8.30 (d, J=4.8 Hz, 2H), 8.06 (s, 1H), 7.89 (d, J=8.4 Hz, 2H), 7.85 (d, J=7.9 Hz, 1H), 7.52 (t, J=7.6 Hz, 1H), 7.40 (d, J=8.2 Hz, 3H), 7.15-7.04 (m, 2H), 6.96 (d, J=7.8 Hz, 1H), 6.82 (t, J=7.0 Hz, 1H), 5.60-5.47 (m, 2H), 4.21 (dd, J=4.7, 6.7 Hz, 1H), 3.80-3.68 (m, 2H), 3.51-3.33 (m, 2H), 2.33 (s, 3H). HRMS calcd. for C₃₁H₂₉N₃O₆S (M+H)⁺ 572.1856. found 572.1810.

### Example 187

### 2-(2-((3′-(Aminomethyl)-5′-fluoro-[1,1′-biphenyl]-3-yl)methoxy)phenyl)acetic acid

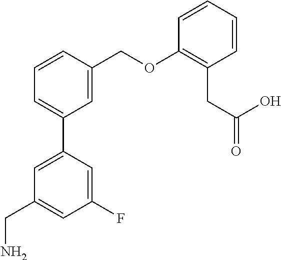 [SMILES: NCc1cc(F)cc(-c2cccc(COc3ccccc3CC(=O)O)c2)c1]

The title compound was prepared from methyl 2-(2-((3-bromobenzyl)oxy)phenyl)acetate (Example 101-A) and tert-butyl 3-fluoro-5-(4,4,5,5-tetramethyl-1,3,2-dioxaborolan-2-yl)benzylcarbamate (Intermediate 24) using the method described in Example 169, DMF was used instead of acetonitrile as solvent. ¹H NMR (400 MHz, DMSO-d₆) δ ppm 7.79 (s, 1H), 7.62 (d, J=3.7 Hz, 1H), 7.53 (br. s., 1H), 7.47 (t, J=6.4 Hz, 2H), 7.41 (d, J=10.1 Hz, 1H), 7.25-7.18 (m, 2H), 7.07-7.00 (m, 2H), 6.90 (t, J=7.5 Hz, 1H), 5.19 (s, 2H), 4.22 (d, J=6.1 Hz, 2H), 3.57 (s, 2H). HRMS calcd. for C₂₂H₂₀FNO₃ (M+H)⁺ 366.1505. found 366.1492.

### Example 188

### (S)-2-(2-((3′-(1-Amino-2-hydroxyethyl)-4-(trifluoromethoxy)-[1,1′-biphenyl]-3-yl)methoxy)phenyl)acetic acid

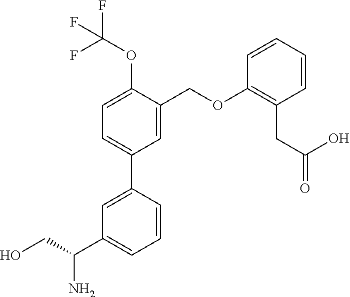 [SMILES: N[C@H](CO)c1cccc(-c2ccc(OC(F)(F)F)c(COc3ccccc3CC(=O)O)c2)c1]

The title compound was prepared from methyl 2-(2-((5-chloro-2-(trifluoromethoxy)benzyl)oxy)phenyl)acetate (Intermediate 84-L) and (S)-tert-butyl (2-hydroxy-1-(3-(4,4,5,5-tetramethyl-1,3,2-dioxaborolan-2-yl)phenyl)ethyl)carbamate (Intermediate 34-A) using the method described in Example 185. ¹H NMR (400 MHz, DMSO-d₆) δ ppm 8.27 (s, 1H), 8.16 (d, J=2.3 Hz, 1H), 7.81 (dd, J=2.3, 8.7 Hz, 1H), 7.71 (d, J=8.0 Hz, 1H), 7.48 (dd, J=1.6, 8.5 Hz, 1H), 7.43 (t, J=7.6 Hz, 1H), 7.33 (d, J=7.8 Hz, 1H), 7.12 (s, 2H), 6.90 (d, J=8.0 Hz, 1H), 6.84 (dt, J=1.0, 7.7 Hz, 1H), 5.26 (s, 2H), 4.18 (dd, J=5.0, 6.4 Hz, 1H), 3.78-3.68 (m, 2H), 3.51-3.33 (m, 2H). HRMS calcd. for C₂₄H₂₂F₃NO₅ (M+H)⁺ 462.1528. found 462.1516.

### Example 189

### (±)-2-(2-((3′-(1,2-Diamino-2-oxoethyl)-[1,1′-biphenyl]-3-yl)methoxy)phenyl)acetic acid

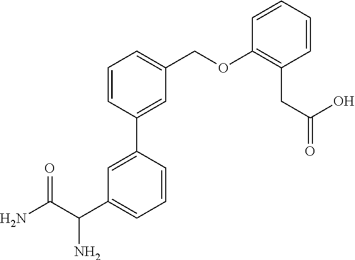 [SMILES: NC(=O)C(N)c1cccc(-c2cccc(COc3ccccc3CC(=O)O)c2)c1]

To a microwave vial was placed methyl 2-(2-((3-(4,4,5,5-tetramethyl-1,3,2-dioxaborolan-2-yl)benzyl)oxy)phenyl)acetate (Example 101-B) (0.1 g, 0.209 mmol) and (±)-2-amino-2-(3-bromophenyl)acetamide (CAS #1105679-26-2) (0.072 g, 0.314 mmol) in MeCN (1 ml). Then K₃PO₄ (2M aq. solution, 0.523 mL, 1.046 mmol) and PdCl₂(dppf).CH₂Cl₂ adduct (CAS #95464-05-4) (7.66 mg, 10.46 μmol) were added and the vial was sealed and heated in a microwave at 140° C. for 60 min. The reaction mixture was cooled to rt and acidified with 1N HCl solution to pH˜3. The organic layer was filtered and 0.5 mL of a 1N LiOH aq. solution was added to the filtrate. The mixture was stirred at rt overnight. The organic layer was filtered and the filtrate was purified by preparative HPLC (Method B) to give the title compound. ¹H NMR (400 MHz, METHANOL-d₄) δ ppm 7.85 (t, J=1.64 Hz, 1H) 7.77-7.81 (m, 2H) 7.60-7.64 (m, 1H) 7.54-7.59 (m, 1H) 7.46-7.52 (m, 3H) 7.20-7.26 (m, 2H) 7.02 (d, J=7.83 Hz, 1H) 6.92 (td, J=7.45, 0.88 Hz, 1H) 5.21 (s, 2H) 5.04 (s, 1H) 3.69 (s, 2H). HRMS calcd. for C₂₃H₂₂N₂O₄ (M+H)⁺ 391.1658. found 391.1656.

### Example 190

The following compounds were prepared with similar methods as described in Example 189, using the boronic ester from Example 101-B and the appropriate aryl bromide.

| | Structure/Chemical Name | Aryl Bromide | ¹H NMR | HRMS |
|---|---|---|---|---|
| 190-A | 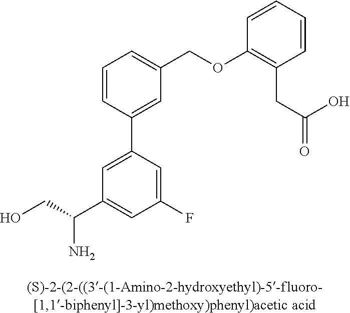 [SMILES: N[C@H](CO)c1cc(F)cc(-c2cccc(COc3ccccc3CC(=O)O)c2)c1] | (S)-2-amino-2- (3-bromo-5- fluorophenyl) ethanol hydrochloride (CAS # 1213947-40-0) | (400 MHz, METHANOL-d₄) δ ppm 7.80 (s, 1 H) 7.59- 7.64 (m, 2 H) 7.47-7.53 (m, 3 H) 7.20-7.27 (m, 3 H) 7.02 (d, J = 7.96 Hz, 1 H) 6.92 (td, J = 7.45, 0.88 Hz, 1 H) 5.21 (s, 2 H) 4.47 (dd, J = 7.52, 4.23 Hz, 1 H) 3.94- 4.00 (m, 1 H) 3.83-3.90 (m, 1 H) 3.69 (s, 2H). | calcd. for C₂₃H₂₂FNO₄ (M + H)⁺ 396.1611, found 396.1596. |
| 190-B a) | 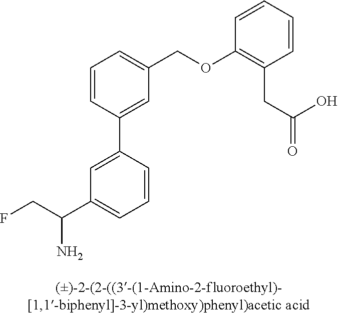 [SMILES: NC(CF)c1cccc(-c2cccc(COc3ccccc3CC(=O)O)c2)c1] | (±)-1-(3- bromophenyl)-2- fluoroethanamine hydrochloride (Intermediate 29-B) | (400 MHz, METHANOL-d₄) δ ppm 8.18 (s, 1 H) 8.05 (s, 1 H) 7.72 (dt, J = 8.02, 1.29 Hz, 1 H) 7.60 (d, J = 7.58 Hz, 1 H) 7.48 (t, J = 7.71 Hz, 1 H) 7.40-7.45 (m, 1 H) 7.35-7.39 (m, 2 H) 7.14-7.21 (m, 2 H) 6.97 (d, J = 7.71 Hz, 1 H) 6.88 (td, J = 7.39, 1.01 Hz, 1 H) 5.13-5.24 (m, 2 H) 4.93-4.99 (m, 0.5 H) 4.84- 4.89 (m, 0.5 H, partially overlapped with solvent) 4.73 (dd, J = 9.92, 4.11 Hz, 0.5 H) 4.59-4.68 (m, 1.5 H) 3.52-3.66 (m, 2 H). | calcd. for C₂₃H₂₂FNO₃ (M + H)⁺ 380.1662, found 380.1656. |
| 190-B b) | (−) and (+)-2-(2-((3′-(1-Amino-2-fluoroethyl)-[1, 1′-biphenyl]-3-yl)methoxy)phenyl)acetic acid | | | |
| | Resolution of the enantiomers of 2-(2-((3′-(1-amino-2-fluoroethyl)-[1, 1′-biphenyl]-3-yl)methoxy)phenyl)acetic acid was | | | |
| | achieved by chiral SFC using a CHIRALPAK ® AS-H column with 20% MeOH (with 10 mM NH₄OH) in CO₂ to give | | | |
| | (−)-2-(2-((3′-(1-amino-2-fluoroethyl)-[1, 1′-biphenyl]-3-yl)methoxy)phenyl)acetic acid (tᵣ = 3.4 min) and (+)-2-(2-((3′- | | | |
| | (1-amino-2-fluoroethyl)-[1, 1′-biphenyl]-3-yl)methoxy)phenyl)acetic acid (tᵣ = 4.8 min). | | | |
| 190-C | 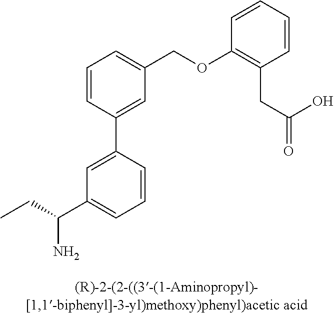 [SMILES: CC[C@@H](N)c1cccc(-c2cccc(COc3ccccc3CC(=O)O)c2)c1] | (R)-1-(3- bromophenyl) propan-1-amine hydrochloride (CAS # 623143-33-9) | (400 MHz, METHANOL-d₄) δ ppm 8.09 (s, 1 H) 8.03 (s, 1 H) 7.66 (dt, J = 7.83, 1.33 Hz, 1 H) 7.60 (d, J = 7.71 Hz, 1 H) 7.43 (td, J = 7.58, 5.31 Hz, 2 H) 7.35-7.39 (m, 1 H) 7.28 (d, J = 7.71 Hz, 1 H) 7.13-7.21 (m, 2 H) 6.97 (d, J = 7.83 Hz, 1 H) 6.87 (td, J = 7.42, 0.95 Hz, 1 H) 5.11- 5.25 (m, 2 H) 4.09 (t, J = 7.45 Hz, 1 H) 3.60-3.67 (m, 1 H) 3.50-3.56 (m, 1 H) 1.95- 2.08 (m, 2 H) 0.89 (t, J = 7.45 Hz, 3 H). | calcd. for C₂₄H₂₅NO₃ (M + H)⁺ 376.1913, found 376.1918. |
| 190-D | 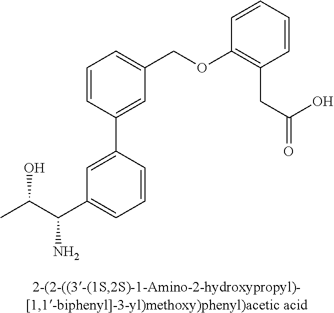 [SMILES: C[C@H](O)[C@@H](N)c1cccc(-c2cccc(COc3ccccc3CC(=O)O)c2)c1] | (1S, 2S)-1- amino-1-(3- bromophenyl) propan-2-ol hydrochloride (CAS # 1269986-58-4) | (400 MHz, METHANOL-d₄) δ ppm 8.08 (s, 1 H) 8.03 (s, 1 H) 7.65-7.70 (m, 1 H) 7.60 (d, J = 7.58 Hz, 1 H) 7.39-7.47 (m, 2 H) 7.35 - 7.39 (m, 1 H) 7.30 (d, J = 7.71 Hz, 1 H) 7.12-7.21 (m, 2 H) 6.96 (d, J = 7.58 Hz, 1 H) 6.86 (td, J = 7.39, 1.01 Hz, 1 H) 5.18 (s, 2H) 4.17- 4.27 (m, 2 H) 3.50-3.65 (m, 2H) 1.11 (d, J = 6.32 Hz, 3H). | LCMS: (ESI+) m/z 392.1 (M + H). |
| 190-E | 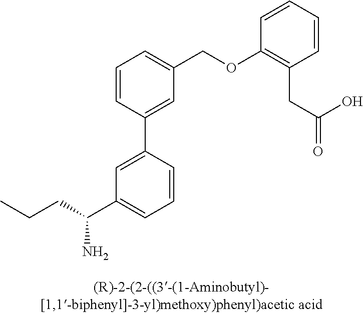 [SMILES: CCC[C@@H](N)c1cccc(-c2cccc(COc3ccccc3CC(=O)O)c2)c1] | (R)-1-(3- bromophenyl) butan-1-amine hydrochloride (CAS # 1391542-02-1) | (400 MHz, METHANOL-d₄) δ ppm 8.14 (s, 1 H) 8.06 (s, 1 H) 7.64-7.69 (m, 1 H) 7.60 (d, J = 7.71 Hz, 1 H) 7.43 (td, J = 7.61, 5.37 Hz, 2 H) 7.33-7.38 (m, 1 H) 7.28 (d, J = 7.71 Hz, 1 H) 7.13- 7.21 (m, 2 H) 6.96 (d, J = 7.83 Hz, 1 H) 6.87 (td, J = 7.39, 1.01 Hz, 1 H) 5.10- 5.26 (m, 2 H) 4.22 (dd, J = 8.84, 6.19 Hz, 1 H) 3.60- 3.69 (m, 1 H) 3.49-3.57 (m, 1 H) 1.91-2.10 (m, 2 H) 1.15-1.42 (m, 2H) 0.90- 0.97 (m, 3H). | calcd. for C₂₅H₂₇NO₃ (M + H)⁺ 390.2069, found 390.2068. |
| 190-F | 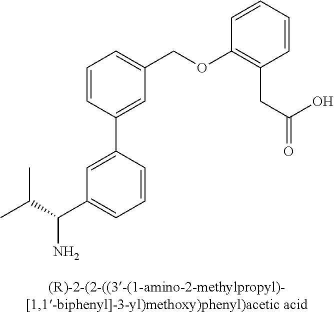 [SMILES: CC(C)[C@@H](N)c1cccc(-c2cccc(COc3ccccc3CC(=O)O)c2)c1] | (R)-1-(3- bromophenyl)-2- methylpropan- 1-amine hydrochloride (CAS # 1391566-64-5) | (400 MHz, METHANOL-d₄) δ ppm 8.10 (s, 1 H) 8.05 (s, 1 H) 7.66 (d, J = 8.08 Hz, 1 H) 7.60 (d, J = 7.71 Hz, 1 H) 7.43 (td, J = 7.64, 4.80 Hz, 2 H) 7.33-7.38 (m, 1 H) 7.25 (d, J = 7.58 Hz, 1 H) 7.14- 7.21 (m, 2 H) 6.97 (d, J = 7.83 Hz, 1 H) 6.82-6.90 (m, 1 H) 5.18-5.25 (m, 1 H) 5.09-5.16 (m, 1 H) 3.87 (d, J = 9.22 Hz, 1 H) 3.63- 3.70 (m, 1 H) 3.48-3.55 (m, 1 H) 2.32 (dt, J = 9.19, 6.71 Hz, 1 H) 1.16 (d, J = 6.57 Hz, 3 H) 0.80 (d, J = 6.82 Hz, 3 H). | calcd. for C₂₅H₂₇NO₃ (M + H)⁺ 390.2069, found 390.2065 |
| 190-G | 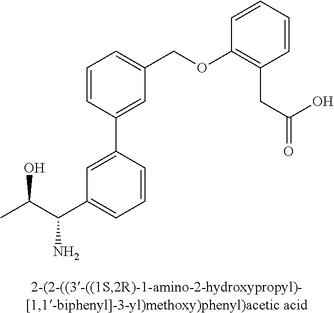 [SMILES: C[C@@H](O)[C@@H](N)c1cccc(-c2cccc(COc3ccccc3CC(=O)O)c2)c1] | (1S, 2R)-1- amino-1-(3- bromophenyl) propan-2-ol hydrochloride (CAS # 1213963-58-6) | (400 MHz, METHANOL-d₄) δ ppm 8.15 (s, 1 H) 8.08 (s, 1 H) 7.67-7.72 (m, 1 H) 7.61 (d, J = 7.96 Hz, 1 H) 7.43 (dt, J = 10.23, 7.71 Hz, 2 H) 7.34-7.38 (m, 1 H) 7.30 (d, J = 7.71 Hz, 1 H) 7.12-7.20 (m, 2H) 6.96 (d, J = 7.71 Hz, 1 H) 6.86 (td, J = 7.39, 1.01 Hz, 1 H) 5.18 (s, 2 H) 4.23-4.30 (m, 2 H) 3.50-3.66 (m, 2H) 1.12 (d, J = 6.19 Hz, 3H). | calcd. for C₂₄H₂₅NO₄ (M + H)⁺ 392.1862, found 392.1853. |
| 190-H | 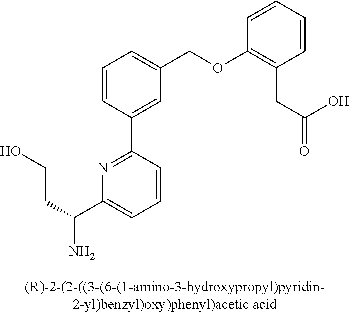 [SMILES: N[C@H](CCO)c1cccc(-c2cccc(COc3ccccc3CC(=O)O)c2)n1] | (R)-3-amino-3-(6- bromopyridin-2- yl)propan-1-ol hydrochloride (CAS# 1213483-26-1) | (400 MHz, METHANOL-d₄) δ ppm 8.68 (s, 1 H) 7.84- 7.93 (m, 3 H) 7.40-7.49 (m, 2 H) 7.31-7.37 (m, 1 H) 7.10-7.19 (m, 2 H) 6.94 (d, J = 7.45 Hz, 1 H) 6.86 (td, J = 7.39, 1.01 Hz, 1 H) 5.21 (s, 2 H) 4.61 (dd, J = 7.58, 5.94 Hz, 1 H) 3.71-3.80 (m, 1 H) 3.63-3.70 (m, 1 H) 3.49-3.61 (m, 2H) 2.12- 2.31 (m, 2H). | calcd. for C₂₃H₂₄N₂O₄ (M + H)⁺ 393.1814, found 393.1831. |
| 190-I | 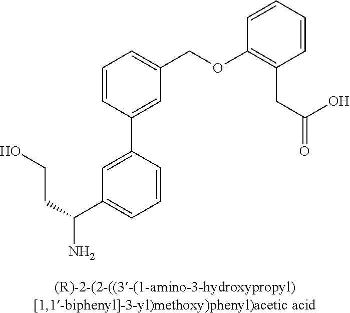 [SMILES: N[C@H](CCO)c1cccc(-c2cccc(COc3ccccc3CC(=O)O)c2)c1] | (R)-3-amino-3-(3- bromophenyl) propan-1-ol hydrochloride (CAS # 11213637-86-5) | (400 MHz, METHANOL-d₄) δ ppm 8.12 (s, 1 H) 8.03 (s, 1 H) 7.66 (dt, J = 7.95, 1.28 Hz, 1 H) 7.60 (d, J = 7.58 Hz, 1 H) 7.43 (q, J = 7.74 Hz, 2 H) 7.35-7.39 (m, 1 H) 7.32 (d, J = 7.58 Hz, 1 H) 7.17 (qd, J = 7.44, 1.65 Hz, 2 H) 6.96 (d, J = 7.70 Hz, 1 H) 6.87 (td, J = 7.40, 0.98 Hz, 1 H) 5.13-5.24 (m, 2 H) 4.41 (t, J = 7.21 Hz, 1 H) 3.59- 3.69 (m, 2 H) 3.45-3.58 (m, 2H) 2.21-2.32 (m, 1 H) 2.09-2.19 (m, 1 H). | calcd. for C₂₄H₂₅NO₄ (M + H)⁺ 392.1862, found 392.1852. |
| 190-J | 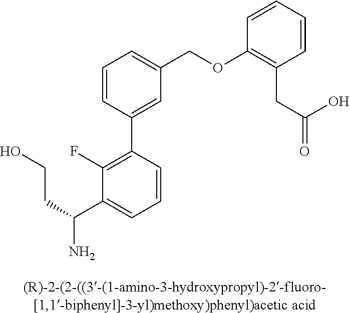 [SMILES: N[C@H](CCO)c1cccc(-c2cccc(COc3ccccc3CC(=O)O)c2)c1F] | (R)-3-amino-3- (3-bromo-2- fluorophenyl) propan-1-ol hydrochloride (CAS # 1272740-39-2) | (400 MHz, METHANOL-d₄) δ ppm 7.84 (s, 1 H) 7.49- 7.54 (m, 1 H) 7.45 (d, J = 1.14 Hz, 4 H) 7.28-7.33 (m, 1 H) 7.20 (d, J = 7.45 Hz, 1 H) 7.11-7.17 (m, 1 H) 6.95 (d, J = 7.58 Hz, 1 H) 6.83-6.90 (m, 1 H) 5.18 (s, 2 H) 4.68 (t, J = 7.20 Hz, 1 H) 3.70 (dt, J = 10.99, 5.49 Hz, 1 H) 3.55-3.63 (m, 3 H) 2.07-2.32 (m, 2 H). | calcd. for C₂₄H₂₄FNO₄ (M + H)⁺ 410.1768, found 410.1765. |
| 190-K | 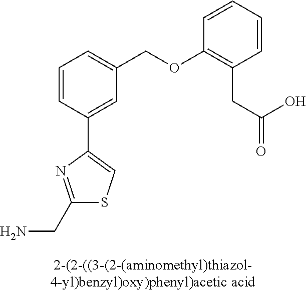 [SMILES: NCc1nc(-c2cccc(COc3ccccc3CC(=O)O)c2)cs1] | (4-bromothiazol-2- yl)methanamine (CAS # 697299-86-8) | (400 MHz, METHANOL-d₄) δ ppm 8.22 (s, 1 H) 7.87 (s, 1 H) 7.80-7.84 (m, 1 H) 7.37-7.44 (m, 2 H) 7.21 (dd, J = 7.39, 1.58 Hz, 1 H) 7.14 (td, J = 7.83, 1.77 Hz, 1 H) 6.95 (d, J = 7.45 Hz, 1 H) 6.87 (td, J = 7.42, 1.07 Hz, 1 H) 5.17 (s, 2 H) 4.27 (s, 2 H) 3.60 (s, 2H). | calcd. for C₁₉H₁₈N₂O₃S (M + H)⁺ 355.1111, found 355.1149. |
| 190-L | 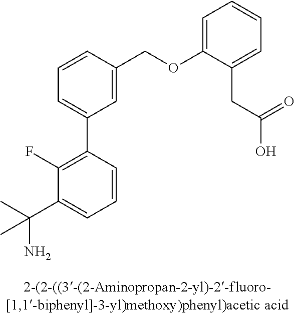 [SMILES: CC(C)(N)c1cccc(-c2cccc(COc3ccccc3CC(=O)O)c2)c1F] | 2-(3-bromo-2- fluorophenyl) propan-2-amine (CAS # 1314710-05-8) | (400 MHz, MeOD) δ 7.74 (d, J = 2.1 Hz, 1H), 7.52- 7.40 (m, 5H), 7.27 (t, J = 7.8 Hz, 1H), 7.20 (dd, J = 7.4, 1.7 Hz, 1H), 7.14 (td, J = 7.8, 1.7 Hz, 1H), 6.96 (dd, J = 8.2, 1.1 Hz, 1H), 6.87 (td, J = 7.4, 1.1 Hz, 1H), 5.16 (s, 2H), 3.57 (s, 2H), 1.71 (s, 6H). | calcd. for C₂₄H₂₅FNO₃ (M + H)⁺ 394.1818, found 394.1805. |

### Example 191

### 2-(2-((3′-(1H-Tetrazol-5-yl)-[1,1′-biphenyl]-3-yl)methoxy)phenyl)acetic acid

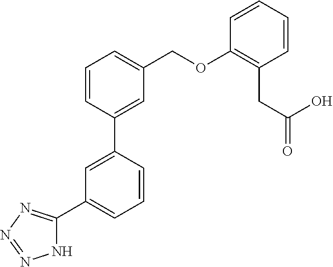 [SMILES: O=C(O)Cc1ccccc1OCc1cccc(-c2cccc(-c3nnn[nH]3)c2)c1]

In a microwave vial was placed methyl 2-(2-((3-bromobenzyl)oxy)phenyl)acetate (Example 101-A) (100 mg, 0.298 mmol) and (3-(1H-tetrazol-5-yl)phenyl)boronic acid (CAS #775351-30-9) (85 mg, 0.448 mmol) in MeCN (1 mL). Then 2M aq. K₃PO₄ (0.746 mL, 1.492 mmol) and Xphos palladacycle (11.02 mg, 0.015 mmol) were added and the vial was sealed and heated in a microwave at 140° C. for 60 min. The reaction mixture was cooled to rt, the organic layer was filtered. 0.5 mL of a 1N LiOH aq. solution was added to the filtrate and stirred overnight. TFA was added until pH˜5. The crude product was purified by HPLC (Method B) to give the title compound. ¹H NMR (400 MHz, METHANOL-d₄) δ ppm 8.34 (t, J=1.64 Hz, 1H) 8.01 (d, J=7.83 Hz, 1H) 7.88-7.93 (m, 1H) 7.85 (s, 1H) 7.64-7.71 (m, 2H) 7.51 (d, J=5.05 Hz, 2H) 7.20-7.28 (m, 2H) 7.05 (d, J=8.08 Hz, 1H) 6.89-6.95 (m, 1H) 5.22 (s, 2H) 3.69 (s, 2H). HRMS calcd. for C₂₂H₁₈N₄O₃ (M+H)⁺ 387.1457. found 387.1456.

### Example 192

### Example 192-A

### Methyl 2-(2-((3′-(N-(tert-butoxycarbonyl)carbamimidoyl)-[1,1′-biphenyl]-3-yl)methoxy)phenyl)acetate

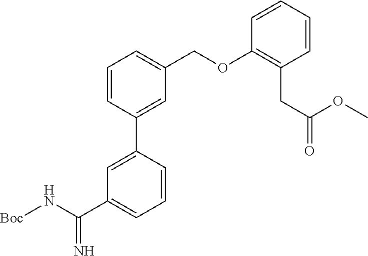 [SMILES: COC(=O)Cc1ccccc1OCc1cccc(-c2cccc(C(=N)NC(=O)OC(C)(C)C)c2)c1]

Into a microwave vial was placed methyl 2-(2-((3-(4,4,5,5-tetramethyl-1,3,2-dioxaborolan-2-yl)benzyl)oxy)phenyl)acetate (Example 101-B) (0.1 g, 0.209 mmol) and tert-butyl ((3-bromophenyl)(imino)methyl)carbamate (Intermediate 28-A) (0.094 g, 0.314 mmol) in MeCN (2 mL). Then K₃PO₄ (2M aq. solution, 0.523 ml, 1.046 mmol) and PdCl₂(dppf).CH₂Cl₂ adduct (CAS #95464-05-4) (7.66 mg, 10.46 μmol) were added and the vial was sealed and heated in a microwave at 110° C. for 60 min. The reaction mixture was cooled to rt, acidified with 1N HCl solution to pH˜3. The organic layer was filtered and purified by preparative HPLC (Method A) to provide the title compound. MS (ESI+) m/z 475.2 (M+H).

### Example 192-B

### 2-(2-((3′-Carbamimidoyl-[1,1′-biphenyl]-3-yl)methoxy)phenyl)acetic acid

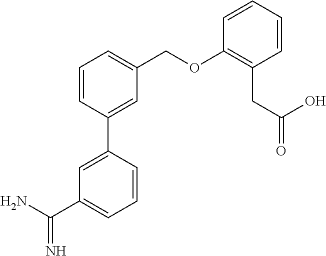 [SMILES: N=C(N)c1cccc(-c2cccc(COc3ccccc3CC(=O)O)c2)c1]

A solution of methyl 2-(2-((3′-(N-(tert-butoxycarbonyl)carbamimidoyl)-[1,1′-biphenyl]-3-yl)methoxy)phenyl)acetate (20 mg, 0.042 mmol) in DCM (1 mL) and TFA (1 mL) was stirred at rt for 60 min. The mixture was concentrated and the residue was dissolved in 2 mL MeOH and 0.5 mL 1N aq. solution of LiOH. The mixture was stirred overnight. TFA was added to adjust to pH˜5. The mixture was filtered and the filtrate was purified by preparative HPLC (Method B) to give the title compound. ¹H NMR (400 MHz, METHANOL-d₄) δ ppm 8.11 (t, J=1.64 Hz, 1H) 8.05 (dt, J=7.74, 1.50 Hz, 1H) 7.86 (s, 1H) 7.76-7.80 (m, 1H) 7.64-7.72 (m, 2H) 7.49-7.52 (m, 2H) 7.20-7.26 (m, 2H) 7.02 (d, J=8.21 Hz, 1H) 6.89-6.95 (m, 1H) 5.23 (s, 2H) 3.69 (s, 2H). HRMS calcd. for C₂₂H₂₀N₂O₃ (M+H)⁺ 361.1552. found 361.1545.

### Example 193

### (S)-2-Amino-2-(3′-((2-(carboxymethyl)phenoxy)methyl)-[1,1′-biphenyl]-3-yl)acetic acid

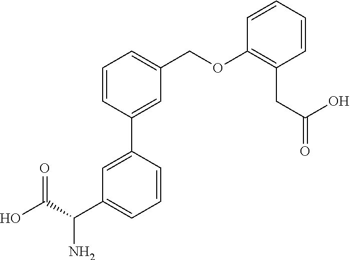 [SMILES: N[C@H](C(=O)O)c1cccc(-c2cccc(COc3ccccc3CC(=O)O)c2)c1]

Into a microwave vial was placed methyl 2-(2-((3-(4,4,5,5-tetramethyl-1,3,2-dioxaborolan-2-yl)benzyl)oxy)phenyl)acetate (Example 101-B) (100 mg, 0.262 mmol) and (S)-methyl 2-amino-2-(3-bromophenyl)acetate (CAS #1213908-25-8) (83 mg, 0.340 mmol) in DMF (2 mL). Then K₃PO₄ (2M aq. solution, 0.654 mL, 1.308 mmol) and Xphos palladacycle (9.66 mg, 0.013 mmol) were added and the vial was sealed and heated in a microwave at 140° C. for 60 min. The reaction mixture was cooled to rt, acidified with 1N HCl solution to pH˜5. The organic layer was filtered. 0.5 mL LiOH (1M aq. solution) was added to the filtrate and the mixture was stirred at room temperature overnight. The mixture was acidified with HCl to pH˜7. The mixture was filtered and the filtrate was purified by HPLC (Method B) to provide the title compound. ¹H NMR (400 MHz, METHANOL-d₄) δ ppm 7.87 (d, J=6.44 Hz, 2H) 7.67 (dt, J=5.53, 2.61 Hz, 1H) 7.59 (d, J=7.20 Hz, 1H) 7.39-7.50 (m, 4H) 7.16-7.23 (m, 2H) 6.99 (d, J=7.96 Hz, 1H) 6.89 (t, J=7.45 Hz, 1H) 5.20 (s, 2H) 4.72 (s, 1H) 3.65 (s, 2H). HRMS calcd. for C₂₃H₂₁NO₅ (M+H)⁺ 392.1498. found 392.1487.

### Example 194

### (R)-2-(2-((3′-(1-Aminobutyl)-[1,1′-biphenyl]-3-yl)methoxy)-3-fluorophenyl)acetic acid

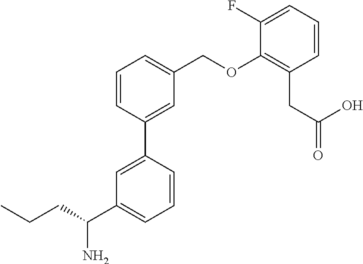 [SMILES: CCC[C@@H](N)c1cccc(-c2cccc(COc3c(F)cccc3CC(=O)O)c2)c1]

The title compound was synthesized in a similar manner as described in Example 75 starting with (R)-tert-butyl (1-(3-(4,4,5,5-tetramethyl-1,3,2-dioxaborolan-2-yl)phenyl)butyl)carbamate (Intermediate 31). ¹H NMR (400 MHz, METHANOL-d₄) δ ppm 8.10 (t, J=1.64 Hz, 1H) 8.07 (s, 1H) 7.68 (dt, J=8.05, 1.22 Hz, 1H) 7.61-7.65 (m, 1H) 7.41-7.49 (m, 2H) 7.34-7.38 (m, 1H) 7.31 (d, J=7.58 Hz, 1H) 6.94-7.05 (m, 3H) 5.20-5.28 (m, 2H) 4.26 (dd, J=8.91, 6.25 Hz, 1H) 3.63-3.69 (m, 1H) 3.51-3.58 (m, 1H) 1.95-2.11 (m, 2H) 1.18-1.43 (m, 2H) 0.92-0.99 (m, 3H). HRMS calcd. for C₂₅H₂₆FNO₃ (M+H)⁺ 408.1975. found 408.1995.

### Example 195

### Example 195-A

### (R)-Methyl 2-(2-((3′-(1-aminobutyl)-2′-fluoro-[1,1′-biphenyl]-3-yl)methoxy)phenyl)acetate

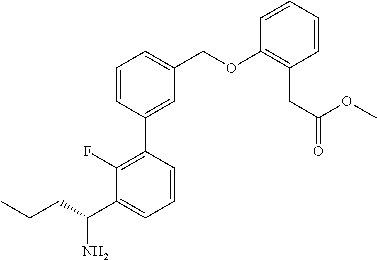 [SMILES: CCC[C@@H](N)c1cccc(-c2cccc(COc3ccccc3CC(=O)OC)c2)c1F]

Into a microwave vial was placed methyl 2-(2-((3-(4,4,5,5-tetramethyl-1,3,2-dioxaborolan-2-yl)benzyl)oxy)phenyl)acetate (Example 101-B) (0.15 g, 0.314 mmol) and (R)-1-(3-bromo-2-fluorophenyl)butan-1-amine hydrochloride (CAS #1213129-43-1) (0.115 g, 0.408 mmol) in acetonitrile (2 mL). K₃PO₄ (2M aq. solution, 0.785 ml, 1.570 mmol) and PdCl₂(dppf).CH₂Cl₂ adduct (CAS #95464-05-4) (0.011 g, 0.016 mmol) were added and the vial was sealed and heated in a microwave at 110° C. for 60 min. The reaction mixture was cooled to rt. The organic layer was filtered and the filtrate was purified by preparative HPLC (Method B) to provide the title compound. MS (ESI+) m/z 422.2 (M+H).

### Example 195-B

### (R)-2-(2-((3′-(1-Aminobutyl)-2′-fluoro-[1,1′-biphenyl]-3-yl)methoxy)phenyl)acetic acid

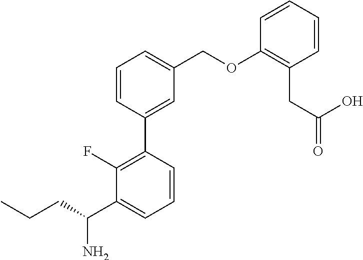 [SMILES: CCC[C@@H](N)c1cccc(-c2cccc(COc3ccccc3CC(=O)O)c2)c1F]

To a solution of (R)-methyl 2-(2-((3′-(1-aminobutyl)-2′-fluoro-[1,1′-biphenyl]-3-yl)methoxy)phenyl)acetate (65 mg, 0.154 mmol) in MeOH (3 mL) was added LiOH (1M aq. solution, 0.463 ml, 0.463 mmol). The mixture was stirred at room temperature for 16 hrs. The mixture was neutralized with HCl to pH˜7 and filtered. The filtrate was purified by preparative HPLC (Method B) to provide the title compound. ¹H NMR (400 MHz, METHANOL-d₄) δ ppm 7.71 (s, 1H) 7.38-7.53 (m, 5H) 7.24-7.29 (m, 1H) 7.21 (dd, J=7.45, 1.64 Hz, 1H) 7.13 (td, J=7.83, 1.64 Hz, 1H) 6.95 (d, J=7.45 Hz, 1H) 6.86 (td, J=7.42, 1.07 Hz, 1H) 5.17 (s, 2H) 4.35 (t, J=7.33 Hz, 1H) 3.57 (s, 2H) 1.86 (q, J=7.58 Hz, 2H) 1.22-1.45 (m, 2H) 0.92-0.98 (m, 3H). HRMS calcd. for C₂₅H₂₆FNO₃ (M+H)⁺ 408.1975. found 408.1984.

### Example 196

The following compounds were prepared with similar methods as described in Example 195, using the boronic ester from Example 101-B and the appropriate aryl bromide.

| | Structure / Chemical Name | Aryl Bromide | ¹H NMR | HRMS |
|---|---|---|---|---|
| 196-A | 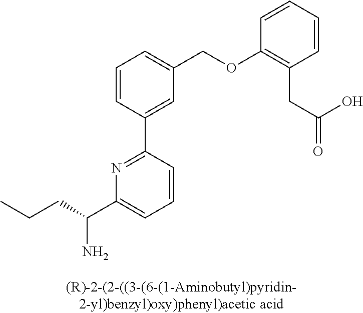 [SMILES: CCC[C@@H](N)c1cccc(-c2cccc(COc3ccccc3CC(=O)O)c2)n1] | (R)-1-(6- bromopyridin-2- yl)butan-1- amine hydrochloride (CAS # 1391554-33-8) | (400 MHz, METHANOL-d₄) δ ppm 8.53 (s, 1 H) 7.92 (dt, J = 7.11, 1.88 Hz, 1 H) 7.83- 7.87 (m, 2 H) 7.43-7.51 (m, 2H) 7.30 (quin, J = 4.39 Hz, 1 H) 7.19 (dd, J = 7.33, 1.64 Hz, 1 H) 7.14 (td, J = 7.83, 1.77 Hz, 1 H) 6.95 (d, J = 7.45 Hz, 1 H) 6.86 (td, J = 7.39, 1.01 Hz, 1 H) 5.20 (s, 2 H) 4.30 (t, J = 6.88 Hz, 1 H) 3.51-3.62 (m, 2H) 1.84-2.02 (m, 2H) 1.29- 1.50 (m, 2 H) 0.96 (t, J = 7.39 Hz, 3 H). | calcd. for C₂₄H₂₆FN₂O₃ (M+H)⁺ 391.2022, found 391.2039. |
| 196-B | 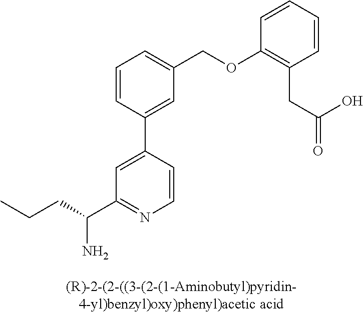 [SMILES: CCC[C@@H](N)c1cc(-c2cccc(COc3ccccc3CC(=O)O)c2)ccn1] | (R)-1-(4- bromopyridin-2- yl)butan-1- amine hydrochloride (CAS # 1259761-20-0) | (400 MHz, METHANOL-d₄) δ ppm 8.57 (d, J = 5.31 Hz, 1 H) 8.19 (d, J = 1-01 Hz, 1 H) 8.15 (s, 1 H) 7.69-7.76 (m, 2 H) 7.47-7.53 (m, 2 H) 7.13-7.22 (m, 2 H) 6.96 (d, J = 7.71 Hz, 1 H) 6.88 (td, J = 7.39, 1.01 Hz, 1 H) 5.16- 5.27 (m, 2 H) 4.34 (dd, J = 8.15, 6.51 Hz, 1 H) 3.60 (q, J = 15.66 Hz, 2 H) 1.92- 2.12 (m, 2 H) 1.20-1.47 (m, 2H) 0.91-0.98 (m, 3 H). | calcd. for C₂₄H₂₆N₂O₃ (M + H)⁺ 391.2022, found 391.2027. |
| 196-C | 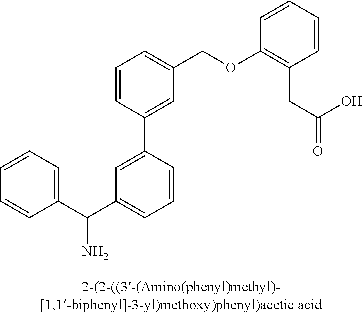 [SMILES: NC(c1ccccc1)c1cccc(-c2cccc(COc3ccccc3CC(=O)O)c2)c1] | (3-bromophenyl) (phenyl) methanamine (CAS # 55095-16-4) | (400 MHz. METHANOL-d₄) δ ppm 7.93 (s, 1 H) 7.88 (s, 1 H) 7.53-7.58 (m, 2 H) 7.41-7.47 (m, 4 H) 7.33- 7.40 (m, 3 H) 7.24-7.29 (m, 1 H) 7.19-7.23 (m, 2 H) 7.14 (td, J = 7.80, 1.71 Hz, 1 H) 6.94-6.98 (m, 1 H) 6.87 (td, J = 7.42, 1.07 Hz, 1 H) 5.37 (s, 1 H) 5.14- 5.22 (m, 2 H) 3.54-3.64 (m, 2H). | calcd. for C₂₈H₂₅NO₃ (M + H)⁺ 424.1907, found 424.1904. |
| 196-D | 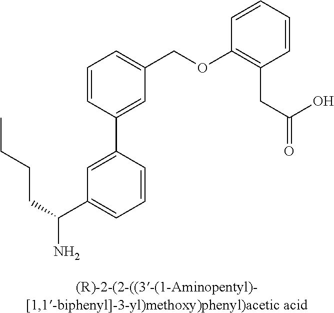 [SMILES: CCCC[C@@H](N)c1cccc(-c2cccc(COc3ccccc3CC(=O)O)c2)c1] | (R)-1-(3- bromophenyl) pentan-1-amine hydrochloride (CAS # 1391534-84-1) | (400 MHz, METHANOL-d₄) δ ppm 7.93 (s, 2 H) 7.55- 7.64 (m, 2 H) 7.39-7.46 (m, 3H) 7.28 (d, J = 7.71 Hz, 1 H) 7.20 (dd, J = 7.45, 1.64 Hz, 1 H) 7.15 (td, J = 7.83, 1.77 Hz, 1 H) 6.96 (d, J = 7.45 Hz, 1 H) 6.87 (td, J = 7.39, 1.01 Hz, 1 H) 5.12- 5.25 (m, 2 H) 4.07 (t, J = 7.33 Hz, 1 H) 3.51-3.66 (m, 2 H) 1.92 (q, J = 7.45 Hz, 2 H) 1.24-1.42 (m, 3H) 1.10- 1.23 (m, 1 H) 0.88 (t, J = 7.20 Hz, 3 H). | calcd. for C₂₆H₂₉NO₃ (M + H)⁺ 404.2226, found 404.2220. |
| 196-E | 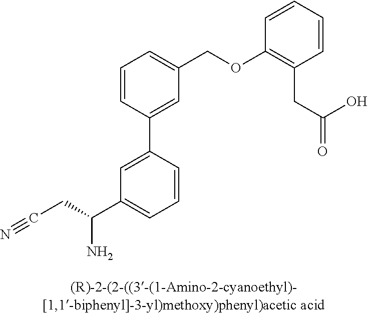 [SMILES: N#CC[C@@H](N)c1cccc(-c2cccc(COc3ccccc3CC(=O)O)c2)c1] | (R)-3-amino-3-(3- bromophenyl) propanenitrile hydrochloride (CAS # 1213204-43-3) | (400 MHz, METHANOL-d₄) δ ppm 8.16-8.19 (m, 1 H) 8.01 (s, 1 H) 7.72 (dt, J = 7.96, 1.26 Hz, 1 H) 7.62 (d, J = 7.58 Hz, 1 H) 7.36- 7.51 (m, 4 H) 7.16-7.21 (m, 2 H) 6.98 (d, J = 7.96 Hz, 1 H) 6.88 (td, J = 7.39, 1.01 Hz, 1 H) 5.14-5.25 (m, 2 H) 4.55 (dd, J = 7.96, 6.57 Hz, 1 H) 3.56-3.67 (m, 2 H) 3.15-3.29 (m, 2H). | calcd. for C₂₄H₂₂N₂O₃ (M + H)⁺ 387.1709, found 387.1700. |
| 196-F | 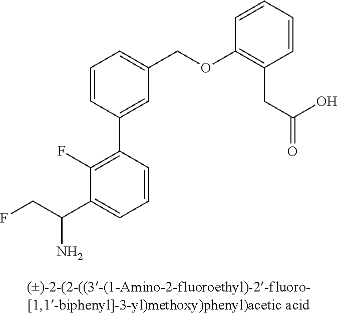 [SMILES: NC(CF)c1cccc(-c2cccc(COc3ccccc3CC(=O)O)c2)c1F] | 1-(3-bromo-2- fluorophenyl)-2- fluoroethanamine hydrochloride (Intermediate 33-B) | (400 MHz, METHANOL-d₄) δ ppm 7.74 (s, 1 H) 7.44- 7.55 (m, 5 H) 7.28-7.34 (m, 1 H) 7.16-7.22 (m, 2 H) 6.99 (d, J = 7.96 Hz, 1 H) 6.89 (td, J = 7.42, 1.07 Hz, 1 H) 5.18 (s, 2 H) 4.62-4.78 (m, 3H) 3.61 (s, 2 H). | calcd. for C₂₃H₂₁F₂NO₃ (M + H)⁺ 398.1568, found 398.1565. |
| 196-G | 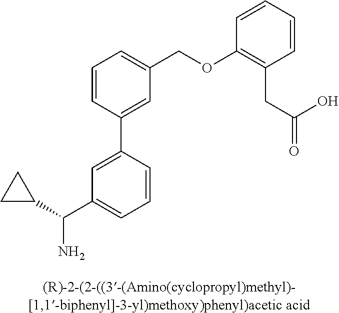 [SMILES: N[C@@H](c1cccc(-c2cccc(COc3ccccc3CC(=O)O)c2)c1)C1CC1] | (R)-(3- bromophenyl) (cyclopropyl) methanamine hydrochloride (CAS # 1213964-00-1) | (400 MHz, METHANOL-d₄) δ ppm 8.25 (t, J = 1.64 Hz, 1 H) 8.10 (s, 1 H) 7.68 (dt, J = 7.93, 1.28 Hz, 1 H) 7.61 (d, J = 7.71 Hz, 1 H) 7.40- 7.47 (m, 2 H) 7.32-7.37 (m, 2 H) 7.14-7.20 (m, 2 H) 6.97 (d, J = 7.83 Hz, 1 H) 6.87 (td, J = 7.39, 1.01 Hz, 1 H) 5.13-5.24 (m, 2 H) 3.51- 3.67 (m, 3 H) 1.50-1.61 (m, 1 H) 0.76-0.84 (m, 1 H) 0.56-0.69 (m, 2H) 0.38- 0.47 (m, 1 H). | calcd. for C₂₅H₂₅NO₃ (M + H)⁺ 388.1907, found 388.1902. |
| 196-H | 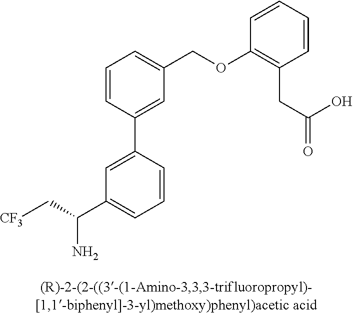 [SMILES: N[C@H](CC(F)(F)F)c1cccc(-c2cccc(COc3ccccc3CC(=O)O)c2)c1] | (R)-1-(3- bromophenyl)- 3, 3, 3- trifluoropropan- 1-amine hydrochloride (CA S# 1259828-51-7) | (400 MHz, METHANOL-d₄) δ ppm 8.25 (s, 1 H) 8.05 (s, 1 H) 7.70-7.74 (m, 1 H) 7.61 (d, J = 7.71 Hz, 1 H) 7.44 (dt, J = 12.22, 7.66 Hz, 2 H) 7.31-7.39 (m, 2 H) 7.14-7.21 (m, 2H) 6.97 (d, J = 7.96 Hz, 1 H) 6.88 (td, J = 7.42, 0.95 Hz, 1 H) 5.19 (q, J = 12.34 Hz, 2 H) 4.58 (t, J = 7.07 Hz, 1 H) 3.60 (q, J = 15.79 Hz, 2 H) 3.00- 3.10 (m, 2 H). | calcd. for C₂₄H₂₂F₃NO₃ (M + H)⁺ 430.1630, found 430.1624. |
| 196-I | 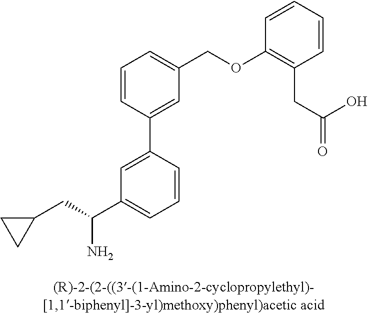 [SMILES: N[C@H](CC1CC1)c1cccc(-c2cccc(COc3ccccc3CC(=O)O)c2)c1] | (R)-1-(3- bromophenyl)-2- cyclopropyl- ethanamine hydrochloride (CAS # 1259833-97-0) | (400 MHz, DMSO-d₆) δ ppm 8.19 (s, 1 H) 8.01 (s, 1 H) 7.61-7.67 (m, 2 H) 7.36- 7.47 (m, 3 H) 7.30 (d, J = 7.58 Hz, 1 H) 7.06-7.13 (m, 2 H) 6.94 (d, J = 7.96 Hz, 1 H) 6.81 (t, J = 7.33 Hz, 1 H) 5.16 (s, 2 H) 4.06 (t, J = 7.14 Hz, 1 H) 3.28-3.40 (m, 2 H) 1.75-1.84 (m, 1 H) 1.66 (dt, J = 13.77. 6.88 Hz, 1 H) 0.57 (d, J = 7.07 Hz, 1 H) 0.24-0.42 (m, 2 H) 0.06- 0.14 (m, 1 H) −0.12-−0.04 (m, 1 H). | calcd. for C₂₆H₂₇NO₃ (M + H)⁺ 402.2069, found 402.2062. |

### Example 197

### (R)-2-(2-((3′-(1-Aminobutyl)-5-((cyclopropylmethyl)amino)-[1,1′-biphenyl]-3-yl)methoxy)phenyl)acetic acid

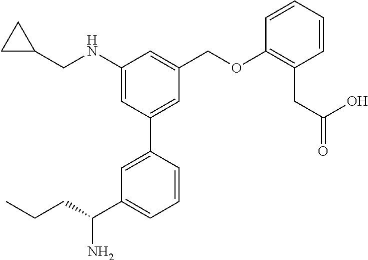 [SMILES: CCC[C@@H](N)c1cccc(-c2cc(COc3ccccc3CC(=O)O)cc(NCC3CC3)c2)c1]

The title compound was synthesized in a similar manner as described in Example 94-B and 94-C, using (R)-tert-butyl (1-(3-(4,4,5,5-tetramethyl-1,3,2-dioxaborolan-2-yl)phenyl)butyl)carbamate (Intermediate 31) in Example 94-B. ¹H NMR (400 MHz, METHANOL-d₄) δ ppm 8.10 (s, 1H) 7.62 (d, J=7.71 Hz, 1H) 7.41 (t, J=7.71 Hz, 1H) 7.35 (s, 1H) 7.24 (d, J=7.71 Hz, 1H) 7.13-7.19 (m, 2H) 6.94 (d, J=7.71 Hz, 1H) 6.83-6.88 (m, 2H) 6.67 (s, 1H) 5.10-5.15 (m, 1H) 5.01-5.07 (m, 1H) 4.21 (dd, J=9.03, 6.13 Hz, 1H) 3.62-3.68 (m, 1H) 3.49-3.55 (m, 1H) 3.03 (d, J=6.69 Hz, 2H) 1.93-2.09 (m, 2H) 1.07-1.39 (m, 3H) 0.90-0.97 (m, 3H) 0.52-0.59 (m, 2H) 0.25-0.31 (m, 2H). HRMS calcd. for C₂₉H₃₄N₂O₃ (M+H)⁺ 459.2642. found 459.2632.

### Example 198

### Example 198-A

### (R)-tert-Butyl 2-(2-((3′-(1-((tert-butoxycarbonyl)amino)butyl)-5-chloro-2′-fluoro-[1,1′-biphenyl]-3-yl)methoxy)phenyl)acetate

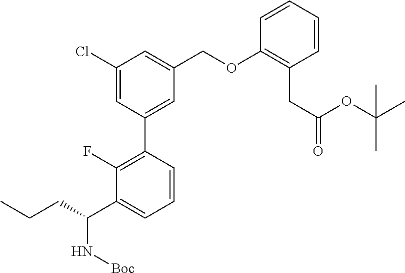 [SMILES: CCC[C@@H](NC(=O)OC(C)(C)C)c1cccc(-c2cc(Cl)cc(COc3ccccc3CC(=O)OC(C)(C)C)c2)c1F]

Into a 50 mL round bottle flask was placed tert-butyl 2-(2-((3-chloro-5-(4,4,5,5-tetramethyl-1,3,2-dioxaborolan-2-yl)benzyl)oxy)phenyl)acetate (Intermediate 91) (0.8 g, 1.744 mmol) and (R)-tert-butyl (1-(3-bromo-2-fluorophenyl)butyl)carbamate (Intermediate 32) (0.785 g, 2.267 mmol) in MeCN (10 mL). Then Xphos palladacycle (0.064 g, 0.087 mmol) and K₃PO₄ (2M aq. solution, 4.36 ml, 8.72 mmol) were added and the flask was heated at 70° C. for 16 hr. The reaction mixture was cooled to rt and the organic layer was separated and filtered, the filtrate was concentrated and purified by flash chromatography (EtOAc-Heptane 0-50%) to provide the title compound. MS (ESI+) m/z 598.2 (M+H).

### Example 198-B

### (R)-tert-Butyl 2-(2-((3′-(1-((tert-butoxycarbonyl)amino)butyl)-2′-fluoro-5-morpholino-[1,1′-biphenyl]-3-yl)methoxy)phenyl)acetate

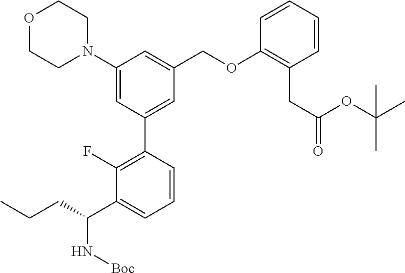 [SMILES: CCC[C@@H](NC(=O)OC(C)(C)C)c1cccc(-c2cc(COc3ccccc3CC(=O)OC(C)(C)C)cc(N3CCOCC3)c2)c1F]

A suspension of (R)-tert-butyl 2-(2-((3′-(1-((tert-butoxycarbonyl)amino)butyl)-5-chloro-2′-fluoro-[1,1′-biphenyl]-3-yl)methoxy)phenyl)acetate (100 mg, 0.167 mmol), morpholine (43.7 mg, 0.502 mmol), BrettPhos palladacycle (CAS #1148148-01-9) (6.68 mg, 8.36 μmol) and Cs₂CO₃ (163 mg, 0.502 mmol) in MeCN (2 mL) was heated in a microwave at 140° C. for 60 min. The organic layer was filtered and the filtrate was purified by flash chromatography (EtOAc-Heptane 0-50%) to give the title compound. MS (ESI+) m/z 649.4 (M+H).

### Example 198-C

### (R)-2-(2-((3′-(1-Aminobutyl)-2′-fluoro-5-morpholino-[1,1′-biphenyl]-3-yl)methoxy)phenyl)acetic acid

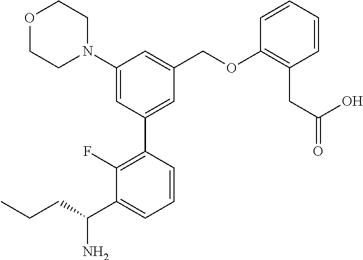 [SMILES: CCC[C@@H](N)c1cccc(-c2cc(COc3ccccc3CC(=O)O)cc(N3CCOCC3)c2)c1F]

The title compound was synthesized in a similar manner as described in Example 77-B. ¹H NMR (400 MHz, METHANOL-d₄) δ ppm 7.52 (td, J=7.52, 1.77 Hz, 1H) 7.37-7.43 (m, 1H) 7.27-7.32 (m, 2H) 7.19 (dd, J=7.39, 1.58 Hz, 1H) 7.14 (td, J=7.83, 1.64 Hz, 1H) 7.09 (s, 1H) 6.99 (s, 1H) 6.94 (d, J=7.58 Hz, 1H) 6.86 (td, J=7.42, 0.95 Hz, 1H) 5.13 (s, 2H) 4.51 (t, J=7.64 Hz, 1H) 3.83-3.88 (m, 4H) 3.57 (s, 2H) 3.19-3.24 (m, 4H) 1.93-2.04 (m, 2H) 1.23-1.45 (m, 2H) 0.97 (t, J=7.39 Hz, 3H). HRMS calcd. for C₂₉H₃₃FN₂O₄(M+H)⁺ 493.2503. found 493.2487.

### Example 199

The following compounds were prepared with similar methods as described in Example 198, using the appropriate amine in Example 198-B.

| | Structure/Chemical Name | Amine | ¹H NMR | HRMS |
|---|---|---|---|---|
| 199-A | 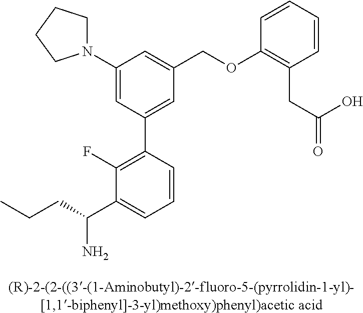 [SMILES: CCC[C@@H](N)c1cccc(-c2cc(COc3ccccc3CC(=O)O)cc(N3CCCC3)c2)c1F] | pyrrolidine | (400 MHz, METHANOL-d₄) δ ppm 7.52 (td, J =7.52, 1.77 Hz, 1 H) 7.35-7.41 (m, 1 H) 7.25- 7.31 (m, 1 H) 7.19 (dd, J = 7.39, 1.58 Hz, 1 H) 7.13 (td, J = 7.83, 1.77 Hz, 1 H) 7.04 (s, 1 H) 6.92-6.97 (m, 1 H) 6.85 (td, J = 7.45, 1.01 Hz, 1 H) 6.67 (s, 1 H) 6.59 (s, 1 H) 5.07-5.14 (m, 2 H) 4.48-4.54 (m, 1 H) 3.58 (s, 2 H) 3.32-3.37 (m, 4 H, overlapped with residual solvent) 1.93-2.09 (m, 6 H) 1.23-1.45 (m, 2 H) 0.93-1.01 (m, 3 H). | calcd. for C₂₉H₃₃FN₂O₃ (M + H)⁺ 477.2553, found 477.2538. |
| 199-B | 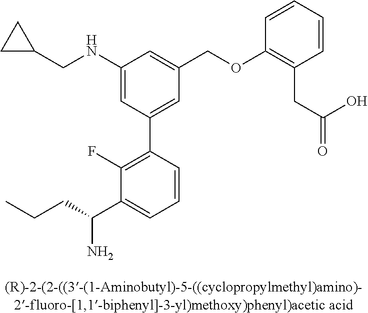 [SMILES: CCC[C@@H](N)c1cccc(-c2cc(COc3ccccc3CC(=O)O)cc(NCC3CC3)c2)c1F] | cyclopropyl methanamine | (400 MHz, METHANOL-d₄) δ ppm 7.49 (td, J = 7.52, 1.77 Hz, 1 H) 7.35-7.41 (m, 1 H) 7.25- 7.30 (m, 1 H) 7.19 (dd, J = 7.45, 1.64 Hz, 1 H) 7.13 (td, J = 7.83, 1.77 Hz, 1 H) 7.04 (s, 1 H) 6.93 (d, J = 7.45 Hz, 1 H) 6.85 (td, J = 7.45, 1.01 Hz, 1 H) 6.77 (s, 1 H) 6.70 (s, 1 H) 5.03- 5.11 (m, 2H) 4.51 (t, J = 7.64 Hz, 1 H) 3.58 (s, 2 H) 3.00 (d, J = 6.69 Hz, 2H) 1.91-2.06 (m, 2 H) 1.23-1.45 (m, 2H) 1.05- 1.16 (m, 1 H) 0.94-1.00 (m, 3 H) 0.50-0.57 (m, 2H) 0.23- 0.29 (m, 2 H). | calcd. for C₂₉H₃₃FN₂O₃ (M+H)+ 477.2553, found 477.2542. |
| 199-C | 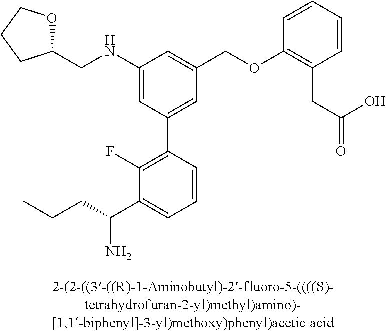 [SMILES: CCC[C@@H](N)c1cccc(-c2cc(COc3ccccc3CC(=O)O)cc(NC[C@@H]3CCCO3)c2)c1F] | (S)- (tetrahydro- furan-2-yl) methanamine (CAS # 7175-81-7) | (400 MHz, METHANOL-d₄) δ ppm 7.49 (td, J = 7.55, 1.71 Hz, 1 H) 7.35-7.40 (m, 1 H) 7.25- 7.31 (m, 1 H) 7.19 (dd, J = 7.39, 1.58 Hz, 1 H) 7.13 (td, J = 7.80, 1.71 Hz, 1 H) 7.05 (s, 1 H) 6.90-6.95 (m, 1 H) 6.85 (td, J = 7.42, 1.07 Hz, 1 H) 6.77 (s, 1 H) 6.71 (s, 1 H) 5.08 (s, 2H) 4.51 (t, J = 7.71 Hz, 1 H) 4.12 (qd, J = 6.82, 4.80 Hz, 1 H) 3.89 (dt, J = 7.86, 6.81 Hz, 1 H) 3.76 (td, J = 7.71, 6.32 Hz, 1 H) 3.58 (s, 2 H) 3.23-3.29 (m, 1 H) 3.15-3.22 (m, 1 H) 1.85-2.10 (m, 5 H) 1.66-1.76 (m, 1 H) 1.22-1.45 (m, 2 H) 0.93-1.01 (m, 3 H). | calcd. for C₃₀H₃₅FN₂O₄ (M + H)⁺ 507.2659, found 507.2652. |

### Example 200

### Example 200-A

### (R)-tert-Butyl 2-(2-((3-(1-((tert-butoxycarbonyl)amino)butyl)-2-fluoro-[1,1′:3′,1″-terphenyl]-5′-yl)methoxy)phenyl)acetate

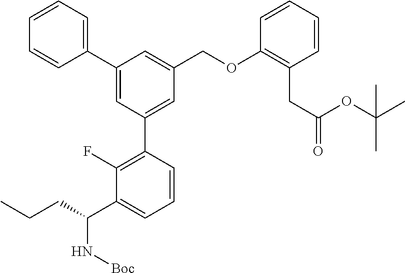 [SMILES: CCC[C@@H](NC(=O)OC(C)(C)C)c1cccc(-c2cc(COc3ccccc3CC(=O)OC(C)(C)C)cc(-c3ccccc3)c2)c1F]

A suspension of (R)-tert-butyl 2-(2-((3′-(1-((tert-butoxycarbonyl)amino)butyl)-5-chloro-2′-fluoro-[1,1′-biphenyl]-3-yl)methoxy)phenyl)acetate (Example 198-A) (50 mg, 0.084 mmol), phenylboronic acid (15.29 mg, 0.125 mmol), SPhos palladacycle (CAS #1375325-64-6) (2.81 mg, 4.18 μmol) in K₃PO₄ (2M aq. solution, 0.125 ml, 0.251 mmol) and MeCN (2 mL) was heated in a microwave at 110° C. for 60 min. The organic layer was filtered and the filtrate was purified by flash chromatography (EtOAc-Heptane 0-50%) to give the title compound. MS (ESI+) m/z 640.4 (M+H).

### Example 200-B

### (R)-2-(2-((3-(1-aminobutyl)-2-fluoro-[1,1′:3′,1″-terphenyl]-5′-yl)methoxy)phenyl)acetic acid

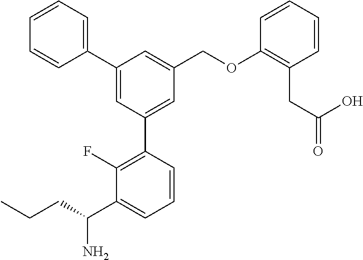 [SMILES: CCC[C@@H](N)c1cccc(-c2cc(COc3ccccc3CC(=O)O)cc(-c3ccccc3)c2)c1F]

The title compound was synthesized in a similar manner as described in Example 77-B starting with (R)-tert-butyl 2-(2-((3-(1-((tert-butoxycarbonyl)amino)butyl)-2-fluoro-[1,1′:3′,1″-terphenyl]-5′-yl)methoxy)phenyl)acetate. ¹H NMR (400 MHz, METHANOL-d₄) δ ppm 7.85 (s, 1H) 7.66-7.72 (m, 4H) 7.62 (td, J=7.58, 1.77 Hz, 1H) 7.41-7.49 (m, 3H) 7.32-7.39 (m, 2H) 7.13-7.23 (m, 2H) 6.99 (d, J=7.45 Hz, 1H) 6.88 (td, J=7.39, 1.01 Hz, 1H) 5.22-5.30 (m, 2H) 4.55 (dd, J=8.65, 6.76 Hz, 1H) 3.60 (s, 2H) 1.94-2.09 (m, 2H) 1.25-1.48 (m, 2H) 0.95-1.02 (m, 3H). HRMS calcd. for C₃₁H₃₀FNO₃ (M+H)⁺ 484.2288. found 484.2283.

### Example 201

### (R)-2-(2-((3′-(1-Aminobutyl)-5-((2-methoxyethyl)amino)-[1,1′-biphenyl]-3-yl)methoxy)phenyl)acetic acid

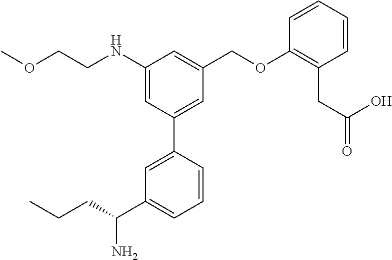 [SMILES: CCC[C@@H](N)c1cccc(-c2cc(COc3ccccc3CC(=O)O)cc(NCCOC)c2)c1]

The title compound was synthesized in a similar manner as described in Example 84 using (R)-tert-butyl (1-(3-(4,4,5,5-tetramethyl-1,3,2-dioxaborolan-2-yl)phenyl)butyl)carbamate (Intermediate 31) in Example 84-B. ¹H NMR (400 MHz, METHANOL-d₄) δ ppm 7.99 (s, 1H) 7.57-7.62 (m, 1H) 7.40 (t, J=7.71 Hz, 1H) 7.29 (s, 1H) 7.24 (d, J=7.58 Hz, 1H) 7.12-7.20 (m, 2H) 6.94 (d, J=7.45 Hz, 1H) 6.83-6.89 (m, 2H) 6.71 (s, 1H) 5.01-5.16 (m, 2H) 4.14 (dd, J=8.27, 6.76 Hz, 1H) 3.59-3.68 (m, 3H) 3.50-3.56 (m, 1H) 3.39 (s, 3H) 3.33-3.38 (m, 2H) 1.87-2.01 (m, 2H) 1.13-1.41 (m, 2H) 0.89-0.98 (m, 3H). HRMS calcd. for C₂₈H₃₄N₂O₄ (M+H)⁺ 463.2597. found 463.2584.

### Example 202

### Example 202-A

### (R)-tert-butyl 2-(2-((3′-(1-((tert-butoxycarbonyl)amino)butyl)-5-cyclopropyl-2′-fluoro-[1,1′-biphenyl]-3-yl)methoxy)phenyl)acetate

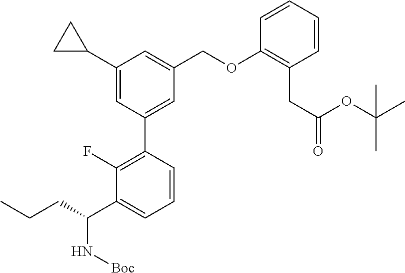 [SMILES: CCC[C@@H](NC(=O)OC(C)(C)C)c1cccc(-c2cc(COc3ccccc3CC(=O)OC(C)(C)C)cc(C3CC3)c2)c1F]

A suspension of (R)-tert-butyl 2-(2-((3′-(1-((tert-butoxycarbonyl)amino)butyl)-5-chloro-2′-fluoro-[1,1′-biphenyl]-3-yl)methoxy)phenyl)acetate (Example 198-A) (100 mg, 0.167 mmol), cyclopropylboronic acid (28.7 mg, 0.334 mmol), Sphos palladacycle (CAS #1375325-64-6) (5.62 mg, 8.36 μmol), Ruphos (CAS #787618-22-8) (3.90 mg, 8.36 μmol) and Cs₂CO₃ (163 mg, 0.502 mmol) in Dioxane (2.5 mL) and water (0.5 mL) was heated in a microwave at 110° C. for 60 min. The organic layer was filtered and the filtrate was purified by flash chromatography (EtOAc-Heptane 0-50%) to give the title compound. MS (ESI+) m/z 626.4 (M+Na).

### Example 202-B

### (R)-2-(2-((3′-(1-Aminobutyl)-5-cyclopropyl-2′-fluoro-[1,1′-biphenyl]-3-yl)methoxy)phenyl)acetic acid

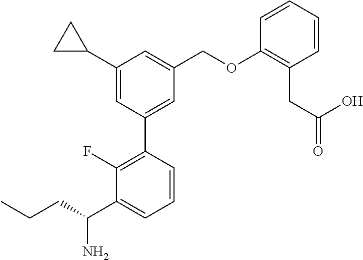 [SMILES: CCC[C@@H](N)c1cccc(-c2cc(COc3ccccc3CC(=O)O)cc(C3CC3)c2)c1F]

The title compound was synthesized in a similar manner as described in Example 77-B starting with (R)-tert-butyl 2-(2-((3′-(1-((tert-butoxycarbonyl)amino)butyl)-5-cyclopropyl-2′-fluoro-[1,1′-biphenyl]-3-yl)methoxy)phenyl)acetate. ¹H NMR (400 MHz, METHANOL-d₄) δ ppm 7.59 (s, 1H) 7.52 (td, J=7.55, 1.71 Hz, 1H) 7.38-7.43 (m, 1H) 7.29-7.33 (m, 1H) 7.12-7.21 (m, 4H) 6.94 (d, J=8.08 Hz, 1H) 6.86 (td, J=7.42, 0.95 Hz, 1H) 5.13 (s, 2H) 4.54 (dd, J=8.53, 6.76 Hz, 1H) 3.58 (s, 2H) 1.94-2.06 (m, 3H) 1.25-1.44 (m, 2H) 0.90-1.02 (m, 5H) 0.73-0.78 (m, 2H). HRMS calcd. for C₂₈H₃₀FNO₃(M+H)⁺ 448.2288. found 448.2273.

### Example 203

### (R)-2-(2-((3′-(1-Aminobutyl)-2′-fluoro-5-(1-methyl-1H-pyrazol-4-yl)-[1,1′-biphenyl]-3-yl)methoxy)phenyl)acetic acid

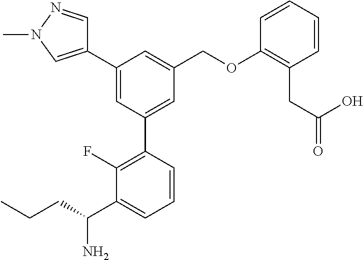 [SMILES: CCC[C@@H](N)c1cccc(-c2cc(COc3ccccc3CC(=O)O)cc(-c3cnn(C)c3)c2)c1F]

The title compound was synthesized in a similar manner as described in Example 200 starting with 1-methyl-4-(4,4,5,5-tetramethyl-1,3,2-dioxaborolan-2-yl)-1H-pyrazole (CAS #761446-44-0) instead of phenylboronic acid. ¹H NMR (400 MHz, DMSO-d₆) δ ppm 8.22 (s, 1H) 7.93 (d, J=0.63 Hz, 1H) 7.68 (s, 1H) 7.62 (s, 1H) 7.53 (t, J=6.38 Hz, 1H) 7.37-7.45 (m, 2H) 7.23-7.29 (m, 1H) 7.20 (ddd, J=7.20, 4.42, 2.53 Hz, 2H) 7.04 (d, J=7.96 Hz, 1H) 6.89 (t, J=6.88 Hz, 1H) 5.19 (s, 2H) 4.17 (t, J=6.76 Hz, 1H) 3.87 (s, 3H) 3.56 (s, 2H) 1.61 (q, J=7.33 Hz, 2H) 1.21-1.42 (m, 2H) 0.87 (t, J=7.33 Hz, 3H). HRMS calcd. for C₂₉H₃₀FN₃O₃(M+H)⁺488.2349. found 488.2331.

### Example 204

### Example 204-A

### tert-Butyl 2-(2-((3′-(1-amino-2-fluoroethyl)-2′-fluoro-5-((((S)-tetrahydrofuran-2-yl)methyl)amino)-[1,1′-biphenyl]-3-yl)methoxy)phenyl)acetate (mixture of diastereomers)

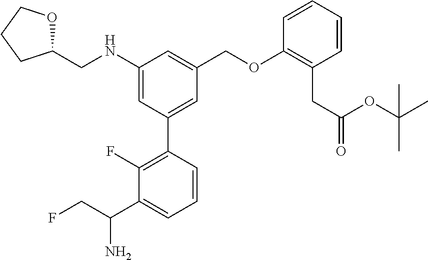 [SMILES: CC(C)(C)OC(=O)Cc1ccccc1OCc1cc(NC[C@@H]2CCCO2)cc(-c2cccc(C(N)CF)c2F)c1]

A suspension of tert-butyl 2-(2-((3-chloro-5-(4,4,5,5-tetramethyl-1,3,2-dioxaborolan-2-yl)benzyl)oxy)phenyl)acetate (Intermediate 91) (100 mg, 0.218 mmol), (S)-(tetrahydrofuran-2-yl)methanamine (CAS #7175-81-7) (33.1 mg, 0.327 mmol), BrettPhos palladacycle (CAS #1148148-01-9) (8.71 mg, 10.90 μmol) and Cs₂CO₃ (213 mg, 0.654 mmol) in MeCN (2 mL) was heated in a microwave at 110° C. for 60 min. The mixture was cooled down and filtered. To the filtrate was added (±)-1-(3-bromo-2-fluorophenyl)-2-fluoroethanamine hydrochloride (Intermediate 33-B) (0.077 g, 0.283 mmol), PdCl₂(dppf).CH₂Cl₂ adduct (CAS #95464-05-4) (7.98 mg, 10.90 μmol), K₃PO₄ (2M aq. solution, 0.545 ml, 1.090 mmol). The vial was sealed and heated in a microwave at 110° C. for 60 min. The reaction mixture was cooled to rt; the organic layer was filtered and purified by flash chromatography (EtOAc-Heptane 0-50%) to give the title compound. MS (ESI+) m/z 553.3 (M+H).

### Example 204-B

### 2-(2-((3′-(1-amino-2-fluoroethyl)-2′-fluoro-5-((((S)-tetrahydrofuran-2-yl)methyl)amino)-[1,1′-biphenyl]-3-yl)methoxy)phenyl)acetic acid (mixture of diastereomers)

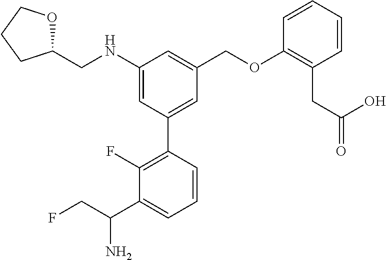 [SMILES: NC(CF)c1cccc(-c2cc(COc3ccccc3CC(=O)O)cc(NC[C@@H]3CCCO3)c2)c1F]

The title compound was synthesized as a mixture of diastereomers in a similar manner as described in Example 77-B starting with tert-butyl 2-(2-((3′-(1-amino-2-fluoroethyl)-2′-fluoro-5-((((S)-tetrahydrofuran-2-yl)methyl)amino)-[1,1′-biphenyl]-3-yl)methoxy)phenyl)acetate (mixture of diastereomers). ¹H NMR (400 MHz, METHANOL-d₄) δ ppm 7.41-7.50 (m, 2H) 7.24-7.29 (m, 1H) 7.14-7.22 (m, 2H) 6.96 (d, J=8.21 Hz, 2H) 6.85-6.90 (m, 1H) 6.80 (s, 1H) 6.73 (s, 1H) 5.08 (s, 2H) 4.59-4.74 (m, 3H) 4.06-4.17 (m, 1H) 3.84-3.94 (m, 1H) 3.73-3.81 (m, 1H) 3.60-3.64 (m, 2H) 3.16-3.26 (m, 2H) 1.85-2.12 (m, 3H) 1.64-1.76 (m, 1H). HRMS calcd. for C₂₈H₃₀F₂N₂O₄(M+H)⁺ 497.2246. found 497.2222.

### Example 205

### Example 205-A

### (±)-tert-Butyl 2-(2-((3′-(1-amino-2-fluoroethyl)-2′-fluoro-5-(methoxymethyl)-[1,1′-biphenyl]-3-yl)methoxy)phenyl)acetate

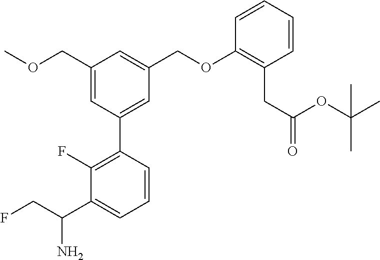 [SMILES: COCc1cc(COc2ccccc2CC(=O)OC(C)(C)C)cc(-c2cccc(C(N)CF)c2F)c1]

Into a microwave vial was placed tert-butyl 2-(2-((3-(methoxymethyl)-5-(4,4,5,5-tetramethyl-1,3,2-dioxaborolan-2-yl)benzyl)oxy)phenyl)acetate (Intermediate 92) (0.08 g, 0.171 mmol), (±)-1-(3-bromo-2-fluorophenyl)-2-fluoroethanamine hydrochloride (Intermediate 33-B) (0.061 g, 0.222 mmol), PdCl₂(dppf).CH₂Cl₂ adduct (CAS #95464-05-4) (6.25 mg, 8.54 μmol) in acetonitrile (2 mL). K₃PO₄ (2M aq. solution, 0.427 ml, 0.854 mmol) was added and the vial was sealed and heated in a microwave at 110° C. for 60 min. The reaction mixture was cooled to rt. The organic layer was filtered and purified by flash chromatography (EtOAc-Heptane 0-50%) to give the title compound. MS (ESI+) m/z 498.3 (M+H).

### Example 205-B

### (±)-2-(2-((3′-(1-Amino-2-fluoroethyl)-2′-fluoro-5-(methoxymethyl)-[1,1′-biphenyl]-3-yl)methoxy)phenyl)acetic acid

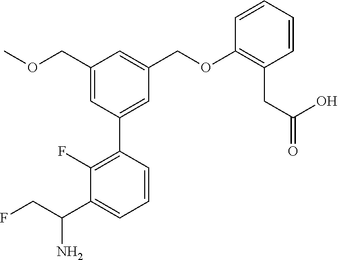 [SMILES: COCc1cc(COc2ccccc2CC(=O)O)cc(-c2cccc(C(N)CF)c2F)c1]

The title compound was synthesized in a similar manner as described in Example 77-B starting with tert-butyl 2-(2-((3′-(1-amino-2-fluoroethyl)-2′-fluoro-5-(methoxymethyl)-[1,1′-biphenyl]-3-yl)methoxy)phenyl)acetate. ¹H NMR (400 MHz, METHANOL-d₄) δ ppm 7.59-7.65 (m, 2H) 7.47-7.53 (m, 3H) 7.35-7.40 (m, 1H) 7.21 (d, J=7.45 Hz, 2H) 7.01 (d, J=7.96 Hz, 1H) 6.91 (td, J=7.42, 0.95 Hz, 1H) 5.19 (s, 2H) 4.92-5.00 (m, 1H) 4.89 (d, J=5.68 Hz, 1H) 4.77 (d, J=5.68 Hz, 1H) 4.54 (s, 2H) 3.65 (s, 2H) 3.41 (s, 3H). HRMS calcd. for C₂₅H₂₅F₂NO₄ (M+H)⁺ 442.1824. found 442.1802.

### Example 206

### (±)-2-(2-((3′-(1-Amino-2-fluoroethyl)-5-(cyclopropylmethoxy)-2′-fluoro-[1,1′-biphenyl]-3-yl)methoxy)phenyl)acetic acid

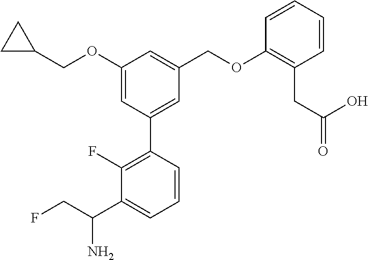 [SMILES: NC(CF)c1cccc(-c2cc(COc3ccccc3CC(=O)O)cc(OCC3CC3)c2)c1F]

The title compound was synthesized in a similar manner as described in Example 205 starting with tert-butyl 2-(2-((3-(cyclopropylmethoxy)-5-(4,4,5,5-tetramethyl-1,3,2-dioxaborolan-2-yl)benzyl)oxy)phenyl)acetate (Intermediate 93). ¹H NMR (400 MHz, METHANOL-d₄) δ ppm 7.59 (td, J=7.64, 1.77 Hz, 1H) 7.46-7.51 (m, 1H) 7.33-7.38 (m, 1H) 7.19-7.25 (m, 3H) 7.07 (s, 1H) 6.98-7.02 (m, 2H) 6.88-6.93 (m, 1H) 5.15 (s, 2H) 4.85-4.97 (m, 2H) 4.75 (d, J=5.56 Hz, 1H) 3.88-3.91 (m, 2H) 3.65 (s, 2H) 1.21-1.32 (m, 1H) 0.58-0.64 (m, 2H) 0.34-0.39 (m, 2H). HRMS calcd. for C₂₇H₂₇F₂NO₄ (M+H)⁺ 468.1981. found 468.1961.

### Example 207

### Example 207-A

### (±)-Methyl 2-(2-((5-(1-amino-2,2,2-trifluoroethyl)-3′-(aminomethyl)-[1,1′-biphenyl]-3-yl)methoxy)phenyl)acetate

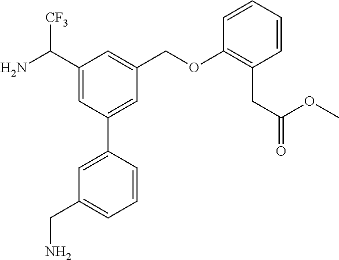 [SMILES: COC(=O)Cc1ccccc1OCc1cc(-c2cccc(CN)c2)cc(C(N)C(F)(F)F)c1]

To a solution of (±)-methyl 2-(2-((3′-(((tert-butoxycarbonyl)amino)methyl)-5-(1-(1,1-dimethylethylsulfinamido)-2,2,2-trifluoroethyl)-[1,1′-biphenyl]-3-yl)methoxy)phenyl)acetate (Intermediate 95) (32 mg, 0.048 mmol) in methanol (0.6 ml) was added HCl 4M in dioxane (0.024 ml, 0.097 mmol). The reaction mixture was stirred at room temperature for 12 hours. The mixture was then concentrated to dryness. The oily residue was then dissolved in DCM and washed with aq. sat. NaHCO₃. The aqueous phase was extracted with DCM. The combined organic phases were dried over a phase separating cartridge and evaporated to obtain crude the title compound. The crude residue was used in the next step without further purification. MS (ESI+) m/z 459.1 (M+H).

### Example 207-B

### (±)-2-(2-((5-(1-Amino-2,2,2-trifluoroethyl)-3′-(aminomethyl)-[1,1′-biphenyl]-3-yl)methoxy)phenyl)acetic acid

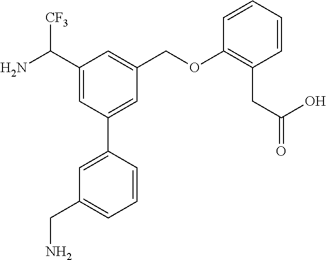 [SMILES: NCc1cccc(-c2cc(COc3ccccc3CC(=O)O)cc(C(N)C(F)(F)F)c2)c1]

To a solution of (±)-methyl 2-(2-((5-(1-amino-2,2,2-trifluoroethyl)-3′-(aminomethyl)-[1,1′-biphenyl]-3-yl)methoxy)phenyl)acetate (15 mg, 0.033 mmol) in THF/MeOH/Water 5:1:1 (0.5 ml/0.1 ml/0.1 ml) was added LiOH.H₂O (0.784 mg, 0.033 mmol). After stirring at room temperature for 16 h, the solvent was evaporated and the compound was purified by preparative HPLC (Waters Sunfire C18-OBD, 5 μm, 30×100 mm, eluent: 5% to 60% CH₃CN in H₂O in 12 min, CH₃CN and H₂O containing 0.1% TFA, flow: 40 mL/min) to yield the title compound. MS (ESI+) m/z 445.1 (M+H).; Rf (DCM:MeOH 8:2)=0.56.

### Example 208

The following compounds were prepared with similar methods as described in Example 207 using the appropriate intermediate from Intermediates 104 as starting materials.

| | | | MS (ESI+) | HPLC | TLC |
|---|---|---|---|---|---|
| | | | m/z | (method C) | Rf |
| | Structure/Chemical Name | Intermediate | (M + H) | t_{R} (Min) | (Eluent) |
| 208-A | 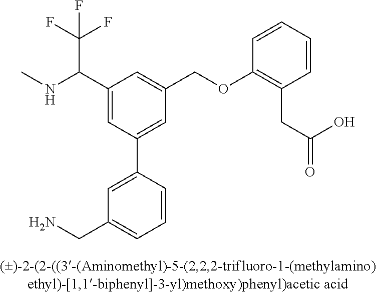 [SMILES: CNC(c1cc(COc2ccccc2CC(=O)O)cc(-c2cccc(CN)c2)c1)C(F)(F)F] | 104-A | 459.1 | 1.387 | 0.17 (DCM/MeOH 9:1 + 5% NH3 7M in MeOH) |
| 208-B | 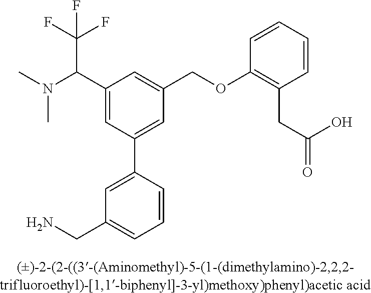 [SMILES: CN(C)C(c1cc(COc2ccccc2CC(=O)O)cc(-c2cccc(CN)c2)c1)C(F)(F)F] | 104-B | 473.2 | 1.471 | 0.23 (DCM/MeOH 9:1 + 5% NH3 7M in MeOH) |
| 208-C | 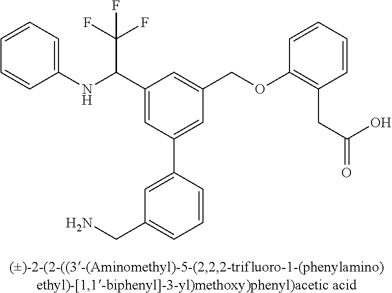 [SMILES: NCc1cccc(-c2cc(COc3ccccc3CC(=O)O)cc(C(Nc3ccccc3)C(F)(F)F)c2)c1] | 104-C | 521.2 | 2.032 | 0.32 (DCM/MeOH 9:1 + 5% NH3 7M in MeOH) |
| 208-D | 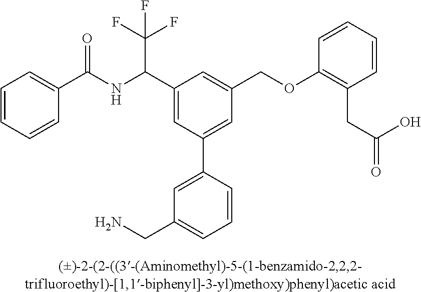 [SMILES: NCc1cccc(-c2cc(COc3ccccc3CC(=O)O)cc(C(NC(=O)c3ccccc3)C(F)(F)F)c2)c1] | 104-D | 549.2 | 1.920 | 0.63 (DCM/MeOH 9:1) |
| 208-E | 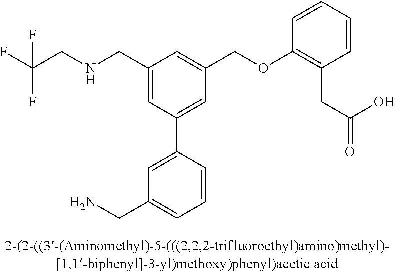 [SMILES: NCc1cccc(-c2cc(CNCC(F)(F)F)cc(COc3ccccc3CC(=O)O)c2)c1] | 104-E | 459.2 | 1.380 | 0.25 (DCM/MeOH 9:1) |
| 208-F | 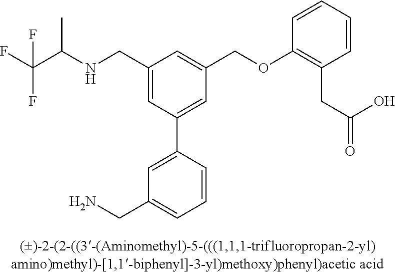 [SMILES: CC(NCc1cc(COc2ccccc2CC(=O)O)cc(-c2cccc(CN)c2)c1)C(F)(F)F] | 104-F | 473.2 | 1.546 | 0.66 (DCM/MeOH 9:1) |
| 208-G | 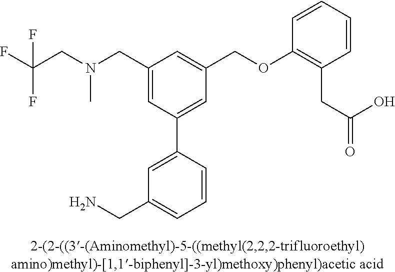 [SMILES: CN(Cc1cc(COc2ccccc2CC(=O)O)cc(-c2cccc(CN)c2)c1)CC(F)(F)F] | 104-G | 473.3 | 1.663 | 0.39 (DCM/MeOH 9:1) |
| 208-H | 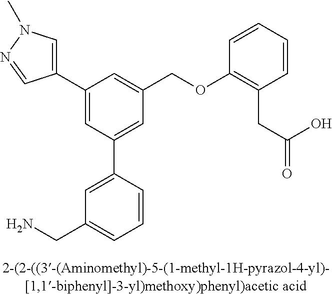 [SMILES: Cn1cc(-c2cc(COc3ccccc3CC(=O)O)cc(-c3cccc(CN)c3)c2)cn1] | 104-H | 428.2 | 1.607 | 0.19 (DCM/MeOH 9:1) |

### Example 209

The following compounds were prepared in analogy to Example 208-H:

| | | MS (ESI+) | HPLC |
|---|---|---|---|
| | | m/z | (method C) |
| | Structure/Chemical Name | (M + H) | t_{R} (Min) |
| 209-A | 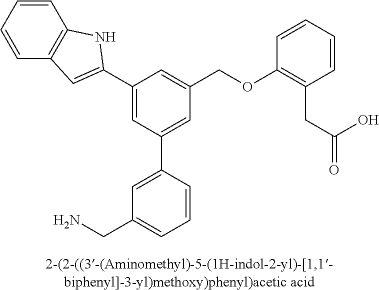 [SMILES: NCc1cccc(-c2cc(COc3ccccc3CC(=O)O)cc(-c3cc4ccccc4[nH]3)c2)c1] | 463.1 | 2.02 min |
| 209-B | 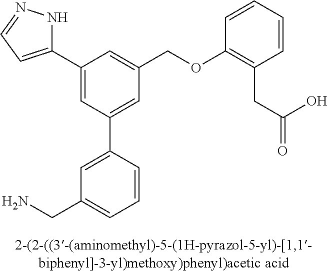 [SMILES: NCc1cccc(-c2cc(COc3ccccc3CC(=O)O)cc(-c3ccn[nH]3)c2)c1] | 414.1 | 1.54 min |
| 209-C | 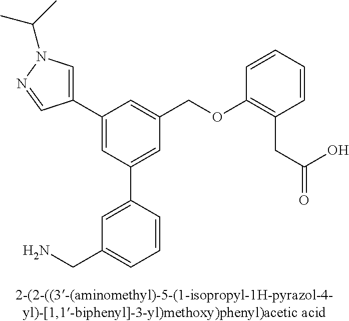 [SMILES: CC(C)n1cc(-c2cc(COc3ccccc3CC(=O)O)cc(-c3cccc(CN)c3)c2)cn1] | 456.3 | 1.78 min |
| 209-D | 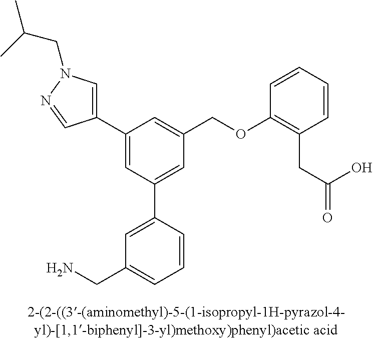 [SMILES: CC(C)Cn1cc(-c2cc(COc3ccccc3CC(=O)O)cc(-c3cccc(CN)c3)c2)cn1] | 470.3 | 1.879 min |
| 209-E | 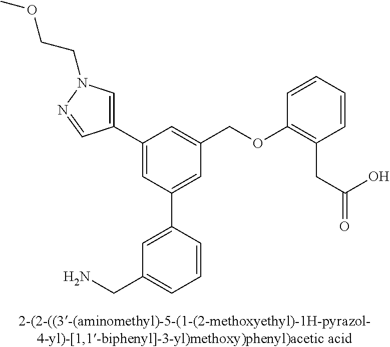 [SMILES: COCCn1cc(-c2cc(COc3ccccc3CC(=O)O)cc(-c3cccc(CN)c3)c2)cn1] | 472.3 | 1.64 min |

### Example 210

### Example 210-A

### (R)-tert-butyl 2-(2-((5-bromo-3′-(1-((tert-butoxycarbonyl)amino)ethyl)-2′-fluoro-[1,1′-biphenyl]-3-yl)methoxy)phenyl)acetate

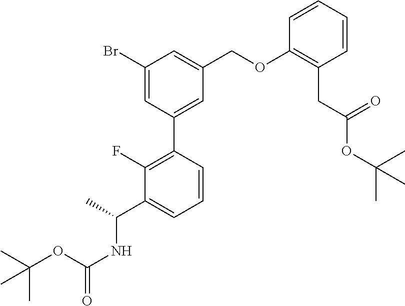 [SMILES: C[C@@H](NC(=O)OC(C)(C)C)c1cccc(-c2cc(Br)cc(COc3ccccc3CC(=O)OC(C)(C)C)c2)c1F]

The title compound was synthesized as described in Example 119-A starting with tert-butyl 2-(2-((3,5-dibromobenzyl)oxy)phenyl)acetate (Intermediate 52) and (R)-tert-butyl (1-(2-fluoro-3-(4,4,5,5-tetramethyl-1,3,2-dioxaborolan-2-yl)phenyl)ethyl)carbamate (Intermediate 27-B). MS (ESI⁺) m/z 614.4, 616.3 (M+H).

### Example 210-B

### (R)-tert-butyl 2-(2-((3′-(1-((tert-butoxycarbonyl)amino)ethyl)-2′-fluoro-5-methyl-[1,1′-biphenyl]-3-yl)methoxy)phenyl)acetate

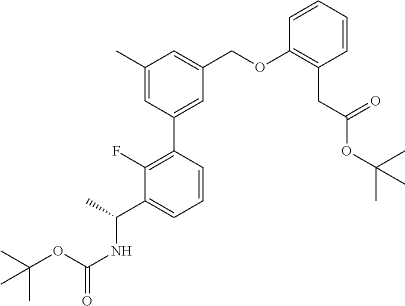 [SMILES: Cc1cc(COc2ccccc2CC(=O)OC(C)(C)C)cc(-c2cccc([C@@H](C)NC(=O)OC(C)(C)C)c2F)c1]

The title compound was synthesized as described in Example 112-A starting with (R)-tert-butyl 2-(2-((5-bromo-3′-(1-((tert-butoxycarbonyl)amino)ethyl)-2′-fluoro-[1,1′-biphenyl]-3-yl)methoxy)phenyl)acetate. MS (ESI⁺) m/z 550.5 (M+H).

### Example 210-C

### (R)-2-(2-((3′-(1-aminoethyl)-2′-fluoro-5-methyl-[1,1′-biphenyl]-3-yl)methoxy)phenyl)acetic acid

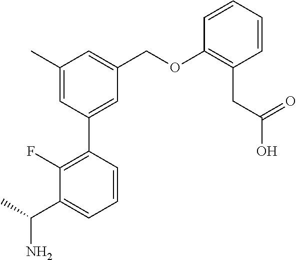 [SMILES: Cc1cc(COc2ccccc2CC(=O)O)cc(-c2cccc([C@@H](C)N)c2F)c1]

The title compound was synthesized as described in Example 114-B starting with (R)-tert-butyl 2-(2-((3′-(1-((tert-butoxycarbonyl)amino)ethyl)-2′-fluoro-5-methyl-[1,1′-biphenyl]-3-yl)methoxy)phenyl)acetate. ¹H NMR (400 MHz, DMSO-d₆) δ ppm 7.54 (t, J=6.44 Hz, 1H) 7.44 (s, 1H) 7.35 (td, J=7.52, 1.64 Hz, 1H) 7.14-7.31 (m, 5H) 7.02 (d, J=7.96 Hz, 1H) 6.82-6.92 (m, 1H) 5.14 (s, 2H) 4.34 (d, J=6.82 Hz, 1H) 3.53 (s, 2H) 2.38 (s, 3H) 1.33 (d, J=6.57 Hz, 3H). HRMS calcd. for C₂₄H₂₄FNO₃ (M+H)⁺ 394.1818. found 394.1824.

### Example 211

### Example 211-A

### (R)-tert-Butyl 2-(2-((6-(3-(1-((tert-butoxycarbonyl)amino)ethyl)-2-fluorophenyl)-1-tosyl-1H-indazol-4-yl)methoxy)phenyl)acetate

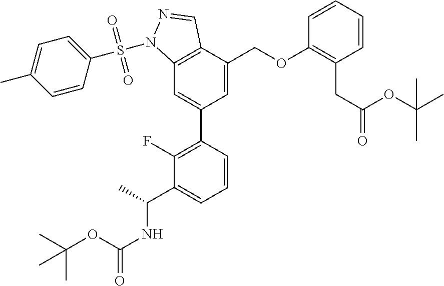 [SMILES: Cc1ccc(S(=O)(=O)n2ncc3c(COc4ccccc4CC(=O)OC(C)(C)C)cc(-c4cccc([C@@H](C)NC(=O)OC(C)(C)C)c4F)cc32)cc1]

The title compound was prepared from tert-butyl 2-(2-((6-bromo-1-tosyl-1H-indazol-4-yl)methoxy)phenyl)acetate (Intermediate 84-J) and (R)-tert-butyl (1-(2-fluoro-3-(4,4,5,5-tetramethyl-1,3,2-dioxaborolan-2-yl)phenyl)ethyl)carbamate (Intermediate 27-B) using the method described in Example 169. MS (ESI−) m/z 728.5 (M−H).

### Example 211-B

### (R)-tert-butyl 2-(2-((6-(3-(1-((tert-butoxycarbonyl)amino)ethyl)-2-fluorophenyl)-1H-indazol-4-yl)methoxy)phenyl)acetate

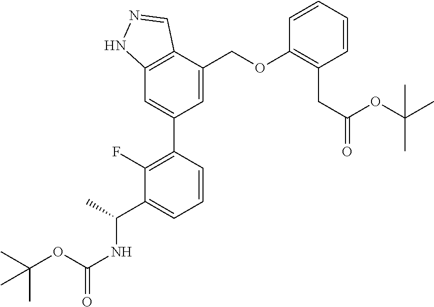 [SMILES: C[C@@H](NC(=O)OC(C)(C)C)c1cccc(-c2cc(COc3ccccc3CC(=O)OC(C)(C)C)c3cn[nH]c3c2)c1F]

Potassium carbonate (101 mg, 3 equiv) was added to a methanol (4 mL) solution of (R)-tert-butyl 2-(2-((6-(3-(1-((tert-butoxycarbonyl)amino)ethyl)-2-fluorophenyl)-1-tosyl-1H-indazol-4-yl)methoxy)phenyl)acetate (150 mg) and the resulting solution was allowed to stir overnight. Water was added and methanol was removed in vacuo. The residue was partitioned between ethyl acetate and water, washed with brine and dried over sodium sulfate. Concentration afforded a brown oil that was used in the next step without additional purification. MS (ESI−) m/z 574.4 (M−H).

### Example 211-C

### (R)-2-(2-((6-(3-(1-aminoethyl)phenyl)-1H-indazol-4-yl)methoxy)phenyl)acetic acid

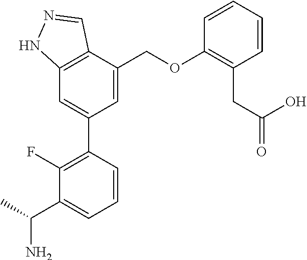 [SMILES: C[C@@H](N)c1cccc(-c2cc(COc3ccccc3CC(=O)O)c3cn[nH]c3c2)c1F]

(R)-tert-butyl 2-(2-((6-(3-(1-((tert-butoxycarbonyl)amino)ethyl)-2-fluorophenyl)-1H-indazol-4-yl)methoxy)phenyl)acetate was treated with 1/1 TFA/DCM (2 mL) for 2 hours, it was then concentrated and purified by HPLC (Method B) to provide the title compound. ¹H NMR (400 MHz, Methanol-d4) b 8.26 (d, J=1.0 Hz, 1H), 7.75-7.61 (m, 2H), 7.57-7.35 (m, 3H), 7.29-7.18 (m, 2H), 7.12 (dd, J=8.2, 1.1 Hz, 1H), 6.93 (td, J=7.4, 1.1 Hz, 1H), 5.52 (s, 2H), 4.85 (m, 1H), 3.67 (br s, 2H), 1.72 (d, J=6.9 Hz, 3H). HRMS calcd. for C₂₄H₂₂FN₃O₃(M+H)⁺ 420.1723. found 420.1716.

### Example 212

### Example 212-A

### (−)-2-(2-((3′-(1-amino-2-fluoroethyl)-2′-fluoro-5-(((R)-tetrahydrofuran-2-yl)methoxy)-[1,1′-biphenyl]-3-yl)methoxy)phenyl)acetic acid

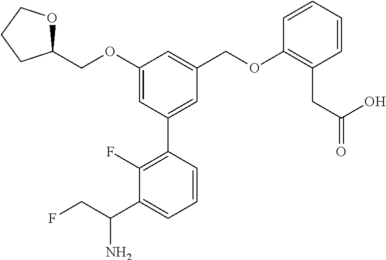 [SMILES: NC(CF)c1cccc(-c2cc(COc3ccccc3CC(=O)O)cc(OC[C@H]3CCCO3)c2)c1F]

The title compound was synthesized in a similar manner as described in Example 205 starting with (R)-tert-butyl 2-(2-((3-((tetrahydrofuran-2-yl)methoxy)-5-(4,4,5,5-tetramethyl-1,3,2-dioxaborolan-2-yl)benzyl)oxy)phenyl)acetate (Intermediate 150) and tert-butyl (1-(3-bromo-2-fluorophenyl)-2-fluoroethyl)carbamate (Intermediate 33-C1). ¹H NMR (400 MHz, METHANOL-d₄) δ ppm 7.55-7.43 (m, 2H), 7.34-7.25 (m, 2H), 7.25-7.14 (m, 2H), 7.09 (dd, J=2.3, 1.3 Hz, 1H), 7.05-6.93 (m, 2H), 6.89 (td, J=7.4, 1.1 Hz, 1H), 5.14 (s, 2H), 4.80-4.67 (m, 2H), 4.68-4.55 (m, 1H), 4.27 (qd, J=6.8, 3.7 Hz, 1H), 4.12-3.76 (m, 4H), 3.62 (s, 2H), 2.15-1.94 (m, 2H), 1.98-1.89 (m, 1H), 1.89-1.77 (m, 1H). HRMS calcd. for C₂₈H₂₉F₂NO₅ (M+H)⁺ 498.2092. found 498.2073.

### Example 212-B

### (+)-2-(2-((3′-(1-amino-2-fluoroethyl)-2′-fluoro-5-(((R)-tetrahydrofuran-2-yl)methoxy)-[1,1′-biphenyl]-3-yl)methoxy)phenyl)acetic acid

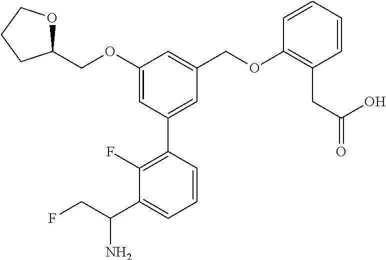 [SMILES: NC(CF)c1cccc(-c2cc(COc3ccccc3CC(=O)O)cc(OC[C@H]3CCCO3)c2)c1F]

The title compound was synthesized in a similar manner as described in Example 205 starting with (R)-tert-butyl 2-(2-((3-((tetrahydrofuran-2-yl)methoxy)-5-(4,4,5,5-tetramethyl-1,3,2-dioxaborolan-2-yl)benzyl)oxy)phenyl)acetate (Intermediate 150) and tert-butyl (1-(3-bromo-2-fluorophenyl)-2-fluoroethyl)carbamate (Intermediate 33-C2). ¹H NMR (400 MHz, METHANOL-d₄) δ ppm 7.55-7.43 (m, 2H), 7.34-7.25 (m, 2H), 7.25-7.14 (m, 2H), 7.09 (dd, J=2.3, 1.3 Hz, 1H), 7.05-6.93 (m, 2H), 6.89 (td, J=7.4, 1.1 Hz, 1H), 5.14 (s, 2H), 4.80-4.67 (m, 2H), 4.68-4.55 (m, 1H), 4.27 (qd, J=6.8, 3.7 Hz, 1H), 4.12-3.76 (m, 4H), 3.62 (s, 2H), 2.15-1.94 (m, 2H), 1.98-1.89 (m, 1H), 1.89-1.77 (m, 1H). HRMS calcd. for C₂₈H₂₉F₂NO₅ (M+H)⁺ 498.2092. found 498.2070.

### Example 213

### (R)-2-(2-((3′-(1-Aminoethyl)-2′-fluoro-5-(pyridin-2-ylmethoxy)-[1,1′-biphenyl]-3-yl)methoxy)phenyl)acetic acid

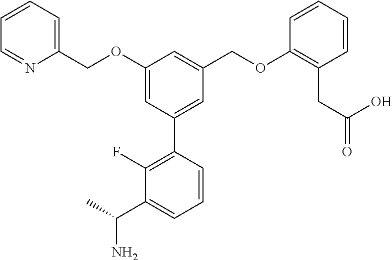 [SMILES: C[C@@H](N)c1cccc(-c2cc(COc3ccccc3CC(=O)O)cc(OCc3ccccn3)c2)c1F]

A solution of (R)-tert-butyl 2-(2-((3′-(1-((tert-butoxycarbonyl)amino)ethyl)-2′-fluoro-5-(pyridin-2-ylmethoxy)-[1,1′-biphenyl]-3-yl)methoxy)phenyl)acetate (Intermediate 115) (58.5 mg, 0.091 mmol) was dissolved in HCl (4M in Dioxane) (0.910 mL, 3.64 mmol) was stirred at room temperature for 2 hours. LCMS shows full conversion to the desired product. Acetonitrile (2 mL) and NH₄OH (0.425 mL, 3.64 mmol) was added. Filtered and the filtrate was purified by preparative HPLC (Sunfire C18; 10-70% ACN/H₂O w/0.1% NH₄OH) to provide title compound. ¹H NMR (400 MHz, Methanol-d₄) δ ppm 8.58-8.52 (m, 1H), 7.88 (td, J=7.7, 1.8 Hz, 1H), 7.65 (d, J=7.8 Hz, 1H), 7.56-7.27 (m, 5H), 7.25-7.06 (m, 4H), 7.00-6.82 (m, 2H), 5.26 (d, J=4.9 Hz, 2H), 5.15 (d, J=3.3 Hz, 2H), 4.73 (q, J=7.0 Hz, 1H), 3.60 (d, J=17.0 Hz, 2H), 1.67 (d, J=6.9 Hz, 3H). HRMS calcd. for C₂₉H₂₇FN₂O₄(M+H)⁺ 487.2033. found 487.2027.

### Example 214

The following compounds were prepared with similar methods as described in Example 213 using the appropriate intermediates from Intermediate 116.

| Ex. | Structure/Chemical Name | ¹H NMR | HRMS |
|---|---|---|---|
| 214-A | 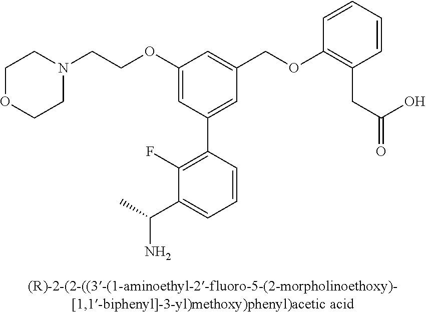 [SMILES: C[C@@H](N)c1cccc(-c2cc(COc3ccccc3CC(=O)O)cc(OCCN3CCOCC3)c2)c1F] | (400 MHz, Methanol-d₄) δ ppm 7.53 (td, J = 7.6, 1.8 Hz, 1H), 7.45 (dd, J = 13.5, 7.0 Hz, 2H), 7.31 (t, J = 7.7 Hz, 1H), 7.25-7.12 (m, 2H), 7.08 (s, 1H), 7.01 (s, 1H), 6.98-6.91 (m, 1H), 6.87 (td, J = 7.5, 1.1 Hz, 1H), 5.16 (s, 2H), 4.72 (q, J = 6.9 Hz, 1H), 4.26 (dt, J = 11.1, 5.5 Hz, 2H), 3.74 (q, J = 5.1 Hz, 4H), 3.60 (d, J = 16.8 Hz, 2H), 2.88 (dt, J = 18.2, 5.6 Hz, 2H), 2.75-2.61 (m, 4H), 1.67 (d, J = 6.9 Hz, 3H). | calcd. for C₂₉H₃₃FN₂O₅ (M + H)⁺ 509.2452, found 509.2461. |
| 214-B | 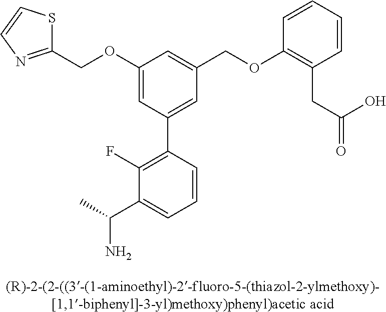 [SMILES: C[C@@H](N)c1cccc(-c2cc(COc3ccccc3CC(=O)O)cc(OCc3nccs3)c2)c1F] | (400 MHz, Methanol-d₄) δ ppm 7.81 (d, J = 3.3 Hz, 1H), 7.63 (d, J = 3.3 Hz, 1H), 7.57- 7.41 (m, 3H), 7.35-7.08 (m, 5H), 6.98-6.82 (m, 2H), 5.48 (s, 2H), 5.16 (s, 2H), 4.71 (q, J = 6.9 Hz, 1H), 3.58 (s, 2H), 1.66 (d, J = 6.9 Hz, 3H). | calcd. for C₂₇H₂₅FN₂O₄S (M + H)⁺ 493.1591, found 493.1541. |
| 214-C | 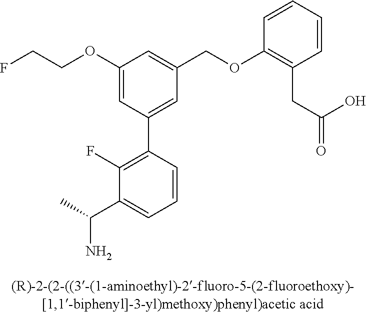 [SMILES: C[C@@H](N)c1cccc(-c2cc(COc3ccccc3CC(=O)O)cc(OCCF)c2)c1F] | (400 MHz, Methanol-d₄) δ ppm7.54 (td, J = 7.6, 1.8 Hz, 1H), 7.49-7.42 (m, 2H), 7.31 (t, J = 7.7 Hz, 1H), 7.24- 7.12 (m, 2H), 7.09 (s, 1H), 7.02 (s, 1H), 6.94 (dd, J = 8.2, 1.1 Hz, 1H), 6.87 (td, J = 7.4, 1.1 Hz, 1H), 5.15 (d, J = 4.6 Hz, 2H), 4.75-4.64 (m, 2H), 4.37-4.23 (m, 2H), 3.58 (s, 2H), 1.67 (d, J = 6.9 Hz, 3H), 1H overlap with solvent signal. | calcd. for C₂₅H₂₅F₂NO₄ (M + H)⁺ 442.1830, found 442.1845. |
| 214-D | 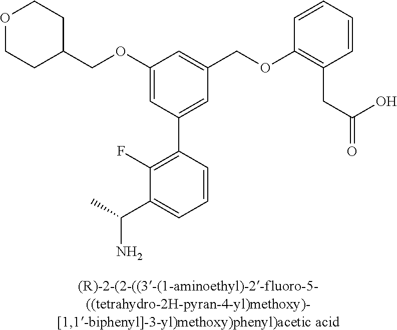 [SMILES: C[C@@H](N)c1cccc(-c2cc(COc3ccccc3CC(=O)O)cc(OCC3CCOCC3)c2)c1F] | (400 MHz, Methanol-d₄) δ ppm 8.39-8.31 (m, 1H), 8.19 (td, J = 7.6, 1.9 Hz, 1H), 8.11-7.96 (m, 4H), 7.89- 7.75 (m, 3H), 7.69 (td, J = 7.4, 1.1 Hz, 1H), 5.97 (s, 2H), 5.16 (q, J = 6.6 Hz, 1H), 4.76- 4.64 (m, 4H), 4.34 (s, 2H), 4.14 (td, J = 11.8, 2.1 Hz, 2H), 2.81 (d, J = 10.5 Hz, 1H), 2.50 (d, J = 12.9 Hz, 2H), 2.19-2.09 (m, 5H). | calcd. for C₂₉H₃₂FNO₅ (M + H)⁺ 494.2343, found 494.2357. |
| 214-E | 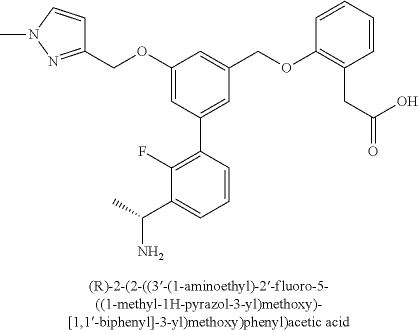 [SMILES: C[C@@H](N)c1cccc(-c2cc(COc3ccccc3CC(=O)O)cc(OCc3ccn(C)n3)c2)c1F] | (400 MHz, Methanol-d₄) δ ppm 7.59-7.40 (m, 4H), 7.30 (t, J = 7.7 Hz, 1H), 7.24- 7.03 (m, 4H), 6.93 (dd, J = 8.2, 1.1 Hz, 1H), 6.87 (td, J = 7.4, 1.1 Hz, 1H), 6.36 (dd, J = 5.1, 2.3 Hz, 1H), 5.14 (d, J = 3.8 Hz, 2H), 5.12 (s, 2H), 4.72 (q, J = 6.9 Hz, 1H), 3.88 (s, 3H), 3.58 (s, 2H), 1.66 (d, J = 6.9 Hz, 3H). | calcd. for C₂₈H₂₈FN₃O₄ (M + H)⁺ 490.2136, found 490.2023. |
| 214-F | 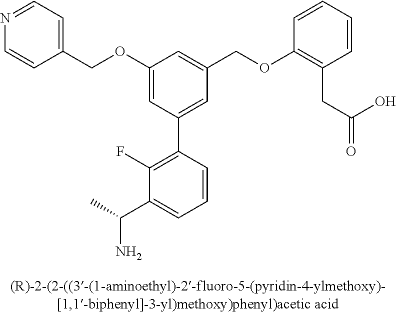 [SMILES: C[C@@H](N)c1cccc(-c2cc(COc3ccccc3CC(=O)O)cc(OCc3ccncc3)c2)c1F] | (400 MHz, Methanol-d₄) δ ppm 8.56-8.51 (m, 2H), 7.59-7.42 (m, 5H), 7.32 (t, J = 7.7 Hz, 1H), 7.25-7.06 (m, 4H), 6.98-6.84 (m, 2H), 5.27 (s, 2H), 5.16 (s, 2H), 4.73 (q, J = 7.0 Hz, 1H), 3.58 (s, 2H), 1.67 (d, J = 6.9 Hz, 3H). | calcd. for C₂₉H₂₇FN₂O₄ (M + H)⁺ 487.2027, found 487.2032. |
| 214-G | 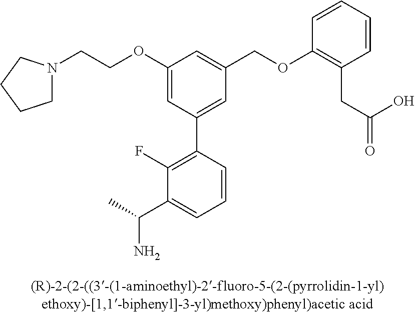 [SMILES: C[C@@H](N)c1cccc(-c2cc(COc3ccccc3CC(=O)O)cc(OCCN3CCCC3)c2)c1F] | (400 MHz, Methanol-d₄ with TFA) δ ppm 7.49-7.64 (m, 2H), 7.35-7.43 (m, 1 H), 7.31 (br. s., 1 H), 7.18-7.26 (m, 3 H), 7.14 (s, 1 H), 6.96-7.01 (m, 1 H), 6.90-6.96 (m, 1 H), 4.78-4.92 (m, 1 H), 4.36- 4.48 (m, 2H), 3.71-3.79 (m, 2H), 3.69 (s, 2H), 3.64- 3.68 (m, 2H), 3.18-3.28 (m, 2 H), 2.13-2.26 (m, 2 H), 2.00-2.12 (m, 2 H), 1.73 (dd, J = 17.1, 6.95 Hz, 3H), (2H overlap with solvent signal. | calcd. for C₂₉H₃₃FN₂O₄ (M + H)⁺ 493.2503, found 493.2491. |
| 214-H | 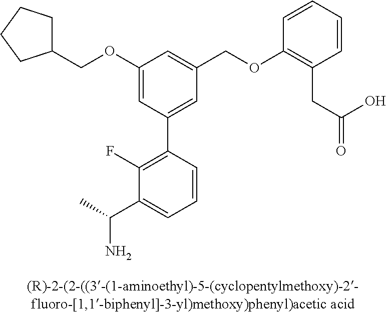 [SMILES: C[C@@H](N)c1cccc(-c2cc(COc3ccccc3CC(=O)O)cc(OCC3CCCC3)c2)c1F] | (400 MHz, Methanol-d₄ with TFA) δ 7.58 (td, J = 7.7, 7.6. 1.8 Hz, 1H), 7.48 (td, J = 7.3, 6.8, 1.7 Hz, 1H), 7.36 (t, J = 7.7, 7.7 Hz, 1H), 7.28-7.18 (m, 3H), 7.10 (dd, J = 2.4, 1.4 Hz, 1H), 7.05-6.98 (m, 2H), 6.92 (td, J = 7.4, 7.3, 1.1 Hz, 1H), 5.16 (s, 2H), 4.98 (d, J = 6.6 Hz, 2H), 4.81 (q, J = 7.0, 7.0, 6.9 Hz, 1H), 3.93 (d, J = 6.9 Hz, 2H), 3.67 (s, 2H), 2.36 (dq, J = 14.8, 7.4, 7.4, 7.4 Hz, 1H), 1.91-1.61 (m, 7H), 1.48-1.35 (m, 2H). | calcd. for C₂₉H₃₂FNO₄ (M + H)⁺ 478.2394, found 478.2383. |
| 214-I | 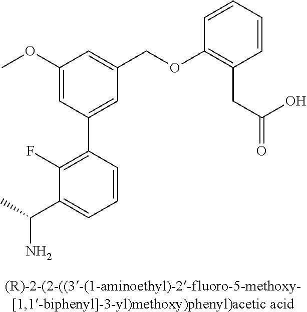 [SMILES: COc1cc(COc2ccccc2CC(=O)O)cc(-c2cccc([C@@H](C)N)c2F)c1] | (400 MHz, Methanol-d₄) δ ppm 7.53 (td, J = 7.58, 1.77 Hz, 1 H), 7.40-7.48 (m, 2 H), 7.28-7.33 (m, 1 H), 7.12- 7.21 (m, 2 H), 7.05 (s, 1 H), 6.92-6.98 (m, 2 H), 6.87 (dd, J = 7.45, 1.01 Hz, 1 H), 5.14- 5.17 (m, 2 H), 4.72 (q, J = 6.82 Hz, 1 H), 3.86 (s, 3 H), 3.58 (s, 2H), 1.67 (d, J = 6.95 Hz, 3 H). | calcd. for C₂₄H₂₄FNO₄ (M + H)⁺ 410.1768, found 410.1774. |
| 214-J | 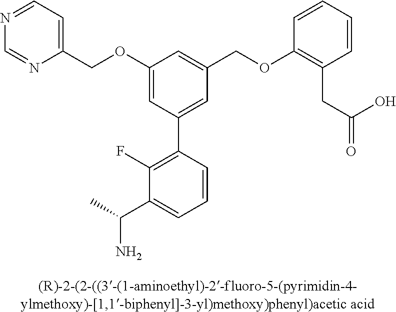 [SMILES: C[C@@H](N)c1cccc(-c2cc(COc3ccccc3CC(=O)O)cc(OCc3ccncn3)c2)c1F] | (400 MHz, Methanol-d₄) δ ppm 9.13 (d, J = 1.4 Hz, 1H), 8.79 (d, J = 5.3 Hz, 1H), 7.75 (ddd, J = 5.3, 1.4, 0.7 Hz, 1H), 7.57-7.42(m, 3H), 7.31 (t, J = 7.7 Hz, 1H), 7.23- 7.08 (m, 4H), 6.95-6.82 (m, 2H), 5.30 (s, 2H), 5.16 (s, (2H), 4.71 (q, J = 6.9 Hz, 1H), 3.57 (s, 2H), 1.66 (d, J = 6.9 Hz, 3H). | calcd. for C₂₈H₂₆FN₃O₄ (M + H)⁺ 488.1980, found 488.1952. |
| 214-K | 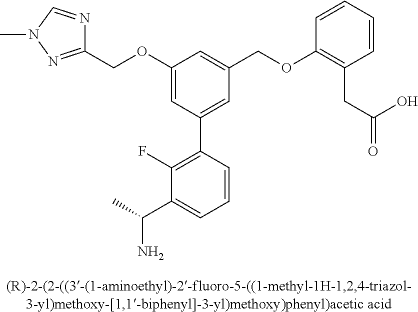 [SMILES: C[C@@H](N)c1cccc(-c2cc(COc3ccccc3CC(=O)O)cc(OCc3ncn(C)n3)c2)c1F] | (400 MHz, Methanol-d₄) δ ppm 8.39 (s, 1H), 7.55-7.41 (m, 3H), 7.29 (t, J = 7.7 Hz, 1H), 7.20 (dd, J = 7.4, 1.7 Hz, 1H), 7.17-7.07 (m, 3H), 6.93 (dd, J = 8.3, 1.1 Hz, 1H), 6.86 (td, J = 7.4, 1.1 Hz, 1H), 5.18 (s, 2H), 5.15 (s, 2H), 4.69 (q, J = 6.9 Hz, 1H), 3.93 (s, 3H), 3.57 (s, 2H), 1.65 (d, J = 6.9 Hz, 3H). | calcd. for C₂₇H₂₇FN₄O₄ (M + H)⁺ 491.2089, found 491.2064. |
| 214-L | 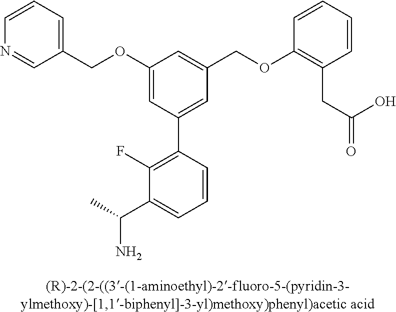 [SMILES: C[C@@H](N)c1cccc(-c2cc(COc3ccccc3CC(=O)O)cc(OCc3cccnc3)c2)c1F] | (400 MHz, Methanol-d₄) δ 8.97 (dt, J = 1.7, 0.8, 0.8 Hz, 1H), 8.85-8.78 (m, 1H), 8.72 (dt, J = 8.1.1.7, 1.7 Hz, 1H), 8.10 (ddd, J = 8.3, 5.8, 0.8 Hz, 1H), 7.56 (dtd, J = 30.1, 7.2, 6.8, 1.7 Hz, 2H), 7.39 (t, J = 7.7, 7.7 Hz, 1H), 7.30 (d, J = 1.6 Hz, 1H), 7.27-7.14 (m, 4H), 6.97-6.88 (m, 2H), 5.46 (s, 2H), 5.22 (s, 2H), 4.82 (q, J = 6.8, 6.8, 6.8 Hz, 1H), 3.68 (s, 2H), 1.71 (d, J = 6.9 Hz, 3H). | calcd. for C₂₉H₂₇FN₂O₄ (M + H)⁺ 487.2033, found 487.2043. |
| 214-M | 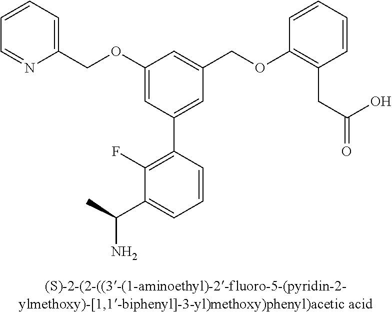 [SMILES: C[C@H](N)c1cccc(-c2cc(COc3ccccc3CC(=O)O)cc(OCc3ccccn3)c2)c1F] | (400 MHz, Methanol-d₄ with TFA) d ppm 8.82 (dt, J = 5.81, 0.76 Hz, 1 H), 8.57 (td, J = 7.93, 1.58 Hz, 1 H), 8.13 (d, J = 8.21 Hz, 1 H), 7.98-8.03 (m, 1 H), 7.60 (td, J = 7.61, 1.71 Hz, 1 H), 7.49- 7.56 (m, 1 H), 7.36-7.44 (m, 1 H), 7.34 (s, 1 H), 7.29 (s, 1 H), 7.17-7.27 (m,3H), 6.90- 6.95 (m, 2H), 5.60 (s, 2 H), 5.23 (s, 2 H), 4.83 (d, J = 6.82 Hz, 1 H), 3.69 (s, 2 H), 1.71 (d, J = 6.82 Hz, 3 H). | calcd. for C₂₉H₂₇FN₂O₄ (M + H)⁺ 487.2033, found 487.1946. |
| 214-N | 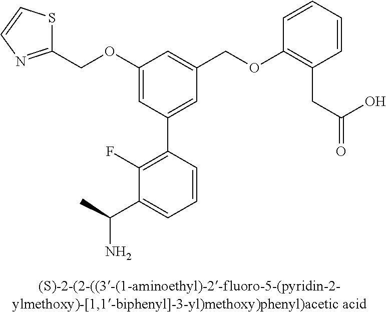 [SMILES: C[C@H](N)c1cccc(-c2cc(COc3ccccc3CC(=O)O)cc(OCc3nccs3)c2)c1F] | (400 MHz, Methanol-d₄) δ ppm 7.81 (d, J = 3.3 Hz, 1H), 7.63 (d, J = 3.3 Hz, 1H), 7.57- 7.41 (m, 3H), 7.35-7.08 (m, 5H), 6.98-6.82 (m, 2H), 5.48 (s, 2H), 5.16 (s, 2H), 4.71 (q, J = 6.9 Hz, 1H), 3.58 (s, 2H), 1.66 (d, J = 6.9 Hz, 3H). | calcd. for C₂₇H₂₅FN₂O₄S (M + H)⁺ 493.1591, found 493.1565. |
| 214-O | 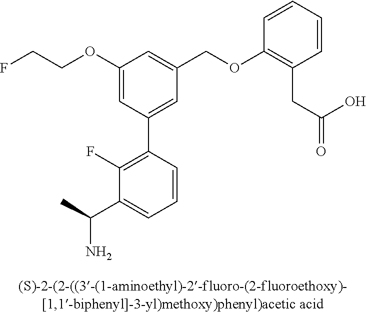 [SMILES: C[C@H](N)c1cccc(-c2cc(COc3ccccc3CC(=O)O)cc(OCCF)c2)c1F] | (400 MHz, Methanol-d₄) δ ppm 7.54 (td, J = 7.6, 1.8 Hz, 1H), 7.49-7.42 (m, 2H), 7.31 (t, J = 7.7 Hz, 1H), 7.24- 7.12 (m, 2H), 7.09 (s, 1H), 7.02 (s, 1H), 6.94 (dd, J = 8.2, 1.1 Hz, 1H), 6.87 (td, J = 7.4, 1.1 Hz, 1H), 5.15 (d, J = 4.6 Hz, 2H), 4.75-4.64 (m, 2H), 4.37-4.23 (m, 2H), 3.58 (s, 2H), 1.67 (d, J = 6.9 Hz, 3H), 1H overlap with solvent signal. | calcd. for C₂₅H₂₅F₂NO₄ (M + H)⁺ 442.1830, found 442.1807. |
| 214-P | 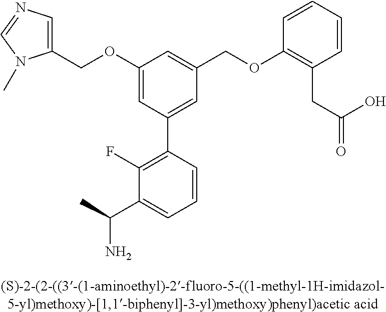 [SMILES: C[C@H](N)c1cccc(-c2cc(COc3ccccc3CC(=O)O)cc(OCc3cncn3C)c2)c1F] | (400 MHz, Methanol-d₄) δ ppm 7.66 (s, 1 H), 7.53 (td, J = 7.58, 1.64 Hz, 1 H), 7.42- 7.48 (m, 2H), 7.28-7.34 (m, 1 H), 7.12-7.22 (m, 3 H), 7.06-7.10 (m, 2 H), 6.94 (d, J = 7.58 Hz, 1 H), 6.87 (td, J = 7.39, 1.01 Hz, 1 H), 5.19 (s, 2H), 5.16 (s, 2 H), 4.72 (q, J = 6.95 Hz, 1 H), 3.76 (s, 3 H), 3.58 (s, 2H), 1.66 (d, J = 6.82 Hz, 3 H). | calcd. for C₂₈H₂₈FN₃O₄ (M + H)⁺ 490.2142, found 490.2124. |
| 214-Q | 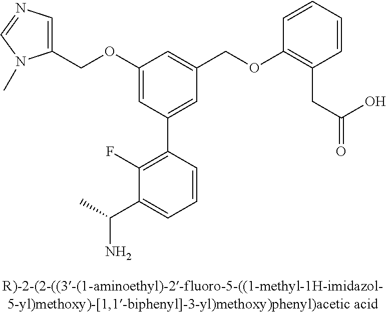 [SMILES: C[C@@H](N)c1cccc(-c2cc(COc3ccccc3CC(=O)O)cc(OCc3cncn3C)c2)c1F] | (400 MHz, Methanol-d4) δ ppm 7.66 (s, 1 H), 7.53 (td, J = 7.58, 1.64 Hz, 1 H), 7.42- 7.48 (m, 2H), 7.28-7.34 (m, 1 H), 7.12-7.22 (m, 3 H), 7.06-7.10 (m, 2 H), 6.94 (d, J = 7.58 Hz, 1 H), 6.87 (td, J = 7.39, 1.01 Hz, 1 H), 5.19 (s, 2H), 5.16 (s, 2H), 4.72 (q, J = 6.95 Hz, 1 H), 3.76 (s, 3 H), 3.58 (s, 2H), 1.66 (d, J = 6.82 Hz, 3 H). | calcd. for C₂₈H₂₈FN₃O₄ (M + H)⁺ 490.2142, found 490.2136. |
| 214-R | 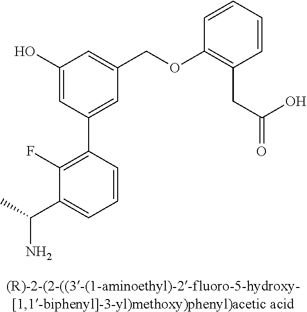 [SMILES: C[C@@H](N)c1cccc(-c2cc(O)cc(COc3ccccc3CC(=O)O)c2)c1F] | (400 MHz, Methanol-d₄) δ ppm 7.50 (td, J = 7.5, 1.8 Hz, 1H), 7.44 (td, J = 7.2, 6.7, 1.7 Hz, 1H), 7.30 (t, J = 7.7 Hz, 1H), 7.25-.22 (m, 1H), 7.19 (dd, J = 7.4, 1.7 Hz, 1H), 7.17-7.11 (m, 1H), 6.97- 6.90 (m, 2H), 6.89-6.83 (m, 2H), 5.10 (s, 2H), 4.72 (q, J = 6.9 Hz, 1H), 3.57 (s, 2H), 1.66 (d, J = 6.9 Hz, 3H). | calcd. for C₂₃H₂₂FNO₄ (M + H)⁺ 396.1611, found 396.1595. |

### Example 215

### 2-(2-((3′-(1-amino-2-fluoroethyl)-2′-fluoro-[1,1′-biphenyl]-3-yl)methoxy)phenyl)acetic acid

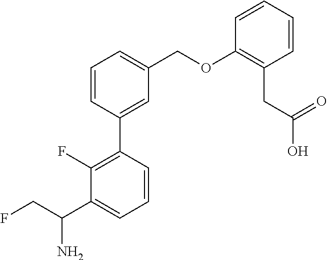 [SMILES: NC(CF)c1cccc(-c2cccc(COc3ccccc3CC(=O)O)c2)c1F]

The title compound was synthesized in a similar manner as described in Example 196-F using the boronic ester from example 101-B and ary bromide Intermediate 33-C2. ¹H NMR (400 MHz, METHANOL-d₄) δ ppm 7.74 (s, 1H) 7.44-7.55 (m, 5H) 7.28-7.34 (m, 1H) 7.16-7.22 (m, 2H) 6.99 (d, J=7.96 Hz, 1H) 6.89 (td, J=7.42, 1.07 Hz, 1H) 5.18 (s, 2H) 4.62-4.78 (m, 3H) 3.61 (s, 2H). HRMS calcd. for C₂₃H₂₁F₂NO₃ (M+H)⁺ 398.1568. found 398.1530.

### Example 216

The following compounds were prepared with similar methods as described in Example 77-B by using appropriate Intermediates.

| Ex. | Structure/Chemical Name | ¹H NMR | HRMS |
|---|---|---|---|
| 216-A | 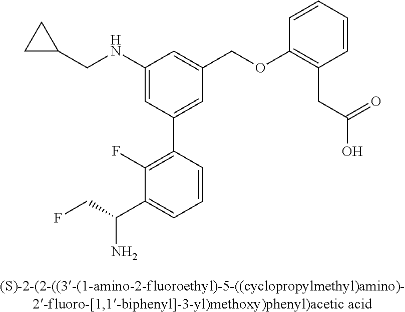 [SMILES: N[C@H](CF)c1cccc(-c2cc(COc3ccccc3CC(=O)O)cc(NCC3CC3)c2)c1F] | (400 MHz, Methanol-d₄) δ ppm 7.53-7.37 (m, 2H), 7.31-7.12 (m, 3H), 7.02- 6.69 (m, 5H), 5.07 (s, 2H), 4.80-4.56 (m, 3H), 3.62 (s, 2H), 2.99 (d, J = 6.7 Hz, 2H), 1.10 (ttt, J = 8.0. 6.7, 4.8 Hz, 1H), 0.58-0.46 (m, 2H), 0.26 (dt, J = 6.0, 4.5 Hz, 2H). | calcd. for C₂₇H₂₈F₂N₂O₃ (M + H)⁺ 467.2140, found 467.2154. |
| 216-B | 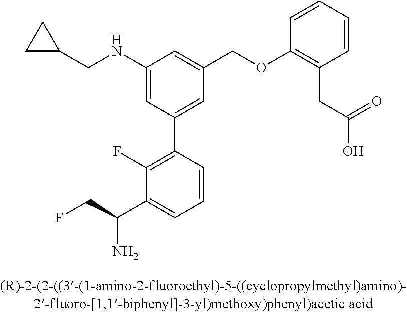 [SMILES: N[C@@H](CF)c1cccc(-c2cc(COc3ccccc3CC(=O)O)cc(NCC3CC3)c2)c1F] | (400 MHz, Methanol-d₄) δ ppm 7.53-7.37 (m, 2H), 7.31-7.12 (m, 3H), 7.02- 6.69 (m, 5H), 5.07 (s, 2H), 4.80-4.56 (m, 3H), 3.62 (s, 2H), 2.99 (d, J = 6.7 Hz, 2H), 1.10 (ttt, J = 8.0, 6.7, 4.8 Hz, 1H), 0.58-0.46 (m, 2H), 0.26 (dt, J = 6.0, 4.5 Hz, 2H). | calcd. for C₂₇H₂₈F₂N₂O₃ (M + H)⁺ 467.2140, found 467.2144. |
| 216-C | 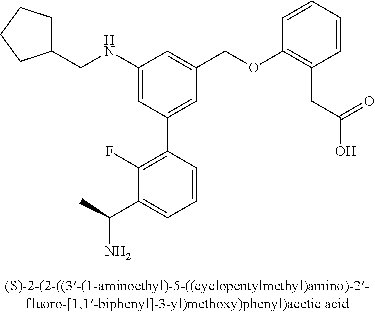 [SMILES: C[C@H](N)c1cccc(-c2cc(COc3ccccc3CC(=O)O)cc(NCC3CCCC3)c2)c1F] | (400 MHz, Methanol-d₄) δ ppm 7.49 (td, J = 7.6, 1.8 Hz, 1H), 7.41 (ddd, J = 8.2, 6.7, 1.8 Hz, 1H), 7.27 (t, J = 7.7 Hz, 1H), 7.19 (dd, J = 7.4, 1.7 Hz, 1H), 7.17-7.09 (m, 1H), 7.02 (dd, J = 2.6, 1.4 Hz, 1H), 6.93 (dd, J = 8.3, 1.1 Hz, 1H), 6.85 (td, J = 7.4, 1.1 Hz, 1H), 6.74 (dd, J = 2.3, 1.3 Hz, 1H), 6.66 (t, J = 1.8 Hz, 1H), 5.07 (s, 2H), 4.70 (q, J = 6.9 Hz, 1H), 3.58 (s, 2H), 3.04 (d, J = 7.2 Hz, 2H), 2.21 (p, J = 7.5 Hz, 1H), 1.92-1.77 (m, 2H), 1.72-1.51 (m, 7H), 1.31 (ddt, J = 14.8, 7.4, 5.3 Hz, 2H). | calcd. for C₂₉H₃₃FN₂O₃ (M + H)⁺ 477.2553, found 477.2543. |
| 216-D | 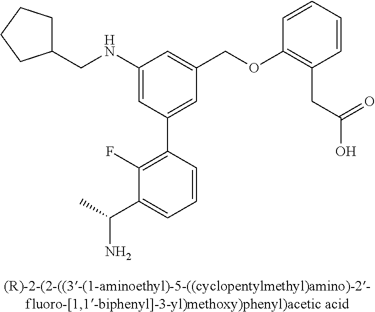 [SMILES: C[C@@H](N)c1cccc(-c2cc(COc3ccccc3CC(=O)O)cc(NCC3CCCC3)c2)c1F] | (400 MHz, Methanol-d₄) δ ppm 7.49 (td, J = 7.6, 1.8 Hz, 1H), 7.41 (ddd, J = 8.2, 6.7, 1.8 Hz, 1H), 7.27 (t, J = 7.7 Hz, 1H), 7.19 (dd, J = 7.4, 1.7 Hz, 1H), 7.17-7.09 (m, 1H), 7.02 (dd, J = 2.6, 1.4 Hz, 1H), 6.93 (dd, J = 8.3, 1.1 Hz, 1H), 6.85 (td, J = 7.4, 1.1 Hz, 1H), 6.74 (dd, J = 2.3, 1.3 Hz, 1H), 6.66 (t, J = 1.8 Hz, 1H), 5.07 (s, 2H), 4.70 (q, J = 6.9 Hz, 1H), 3.58 (s, 2H), 3.04 (d, J = 7.2 Hz, 2H), 2.21 (p, J = 7.5 Hz, 1H), 1.92-1.77 (m, 2H), 1.72-1.51 (m, 7H), 1.31 (ddt, J = 14.8, 7.4, 5.3 Hz, 2H). | calcd. for C₂₉H₃₃FN₂O₃ (M + H)⁺ 477.2553, found 477.2496 |
| 216-E | 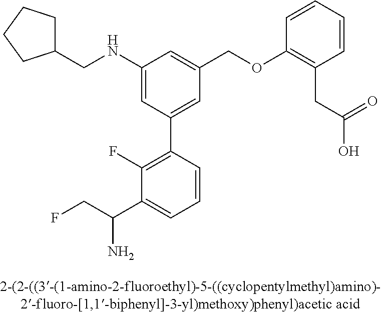 [SMILES: NC(CF)c1cccc(-c2cc(COc3ccccc3CC(=O)O)cc(NCC3CCCC3)c2)c1F] | (400 MHz, Methanol-d₄) δ ppm 7.52-7.40 (m, 2H), 7.27 (t, J = 7.7 Hz, 1H), 7.23- 7.13 (m, 2H), 6.99-6.83 (m, 3H), 6.76 (dd, J = 2.3, 1.4 Hz, 1H), 6.68 (q, J = 1.6 Hz, 1H), 5.07 (s, 2H), 4.73 (qd, J = 8.6, 8.0, 3.4 Hz, 2H), 4.68- 4.57 (m, 1H), 3.62 (s, 2H), 3.04 (d, J = 7.2 Hz, 2H), 2.20 (dt, J = 15.1, 7.5 Hz, 1H), 1.91-1.77 (m, 2H), 1.72- 1.51 (m, 4H), 1.38-1.23 (m, 2H). | calcd. for C₂₉H₃₂F₂N₂O₃ (M + H)⁺ 495.2453, found 495.2451. |
| 216-F | 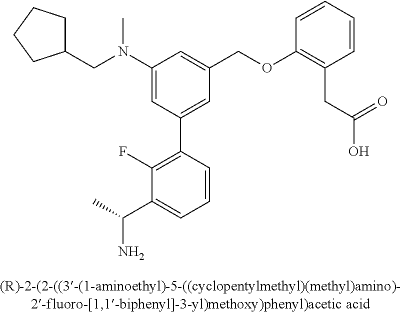 [SMILES: C[C@@H](N)c1cccc(-c2cc(COc3ccccc3CC(=O)O)cc(N(C)CC3CCCC3)c2)c1F] | (400 MHz, Methanol-d₄) δ ppm 7.51 (td, J = 7.5, 1.8 Hz, 1H), 7.46-7.38 (m, 1H), 7.29 (t, J = 7.7 Hz, 1H), 7.19 (dd, J = 7.4, 1.8 Hz, 1H), 7.13 (td, J = 7.8, 1.8 Hz, 1H), 7.07 (s, 1H), 6.96-6.92 (m, 1H), 6.85 (td, J = 7.3, 1.1 Hz, 1H), 6.80 (s, 1H), 6.72 (s, 1H), 5.11 (s, 2H), 4.72 (q, J = 6.9 Hz, 1H), 3.58 (s, 2H), 3.01 (s, 3H), 2.31 (p, J = 7.7 Hz, 1H), 1.80-1.50 (m, 9H), 1.28 (dt, J = 11.8, 7.7 Hz, 2H). 2H overlap with solvent. | calcd. for C₃₀H₃₅FN₂O₃ (M + H)⁺ 491.2704, found 491.2671. |
| 216-G | 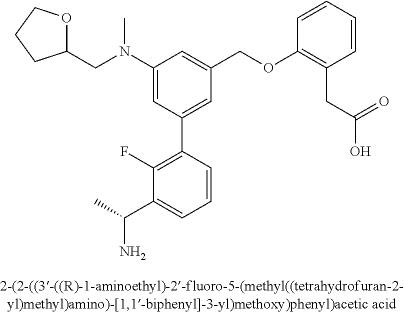 [SMILES: C[C@@H](N)c1cccc(-c2cc(COc3ccccc3CC(=O)O)cc(N(C)CC3CCCO3)c2)c1F] | (400 MHz, Methanol-d₄) δ ppm 7.49 (td, J = 7.6, 1.8 Hz, 1H), 7.46-.38 (m, 1H), 7.27 (t, J = 7.7 Hz, 1H), 7.20 (dd, J = 7.4, 1.7 Hz, 1H), 7.17- 7.06 (m, 2H), 6.98-6.91 (m, 1H), 6.91-6.82 (m, 2H), 6.79 (s, 1H), 5.11 (d, J = 3.2 Hz, 2H), 4.72-4.62 (m, 1H), 4.15 (qd, J = 6.9, 4.2 Hz, 1H), 3.86 (dt, J = 8.2, 6.7 Hz, 1H), 3.73 (td, J = 7.9, 5.8 Hz, 1H), 3.59 (d, J = 10.4 Hz, 2H), 3.54-3.39 (m, 2H), 3.04 (d, J = 5.2 Hz, 3H), 2.07-1.81 (m, 3H), 1.69-1.56 (m, 4H). | calcd. for C₂₉H₃₃FN₂O₄ (M + H)⁺ 493.2497, found 493.2485. |
| 216-H | 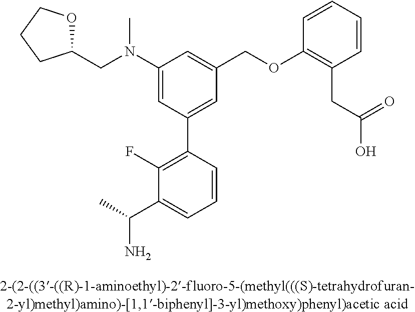 [SMILES: C[C@@H](N)c1cccc(-c2cc(COc3ccccc3CC(=O)O)cc(N(C)C[C@@H]3CCCO3)c2)c1F] | (400 MHz, Methanol-d₄) δ ppm 7.49 (td, J = 7.6, 1.8 Hz, 1H), 7.46-7.38 (m, 1H), 7.27 (t, J = 7.7 Hz, 1H), 7.20 (dd, J = 7.4, 1.7 Hz, 1H), 7.17- 7.06 (m, 2H), 6.98-6.91 (m, 1H), 6.91-6.82 (m, 2H), 6.79 (s, 1H), 5.11 (d, J = 3.2 Hz, 2H), 4.72-4.62 (m, 1H), 4.15 (qd, J = 6.9, 4.2 Hz, 1H), 3.86 (dt, J = 8.2, 6.7 Hz, 1H), 3.73 (td, J = 7.9, 5.8 Hz, 1H), 3.59 (d, J = 10.4 Hz, 2H), 3.54-3.39 (m, 2H), 3.04 (d, J = 5.2 Hz, 3H), 2.07-1.81 (m, 3H), 1.69-1.56 (m, 4H). | calcd. for C₂₉H₃₃FN₂O₄ (M + H)⁺ 493.2497, found 493.2491. |
| 216-I | 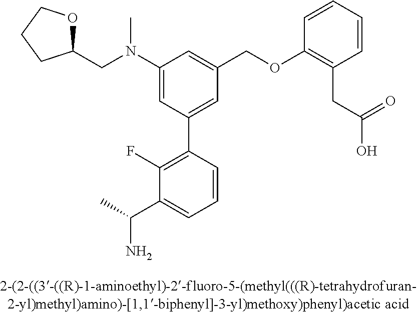 [SMILES: C[C@@H](N)c1cccc(-c2cc(COc3ccccc3CC(=O)O)cc(N(C)C[C@H]3CCCO3)c2)c1F] | (400 MHz, Methanol-d₄) δ ppm 7.49 (td, J = 7.6, 1.8 Hz, 1H), 7.46-7.38 (m, 1H), 7.27 (t, J = 7.7 Hz, 1H), 7.20 (dd, J = 7.4, 1.7 Hz, 1H), 7.17- 7.06 (m, 2H), 6.98-6.91 (m, 1H), 6.91-6.82 (m, 2H), 6.79 (s, 1H), 5.11 (d, J = 3.2 Hz, 2H), 4.72-4.62 (m, 1H), 4.15 (qd, J = 6.9, 4.2 Hz, 1H), 3.86 (dt, J = 8.2, 6.7 Hz, 1H), 3.73 (td, J = 7.9, 5.8 Hz, 1H), 3.59 (d, J = 10.4 Hz, 2H), 3.54-3.39 (m, 2H), 3.04 (d, J = 5.2 Hz, 3H), 2.07-1.81 (m, 3H), 1.69-1.56 (m, 4H). | calcd. for C₂₉H₃₃FN₂O₄ (M + H)⁺ 493.2497, found 493.2493. |
| 216-J | 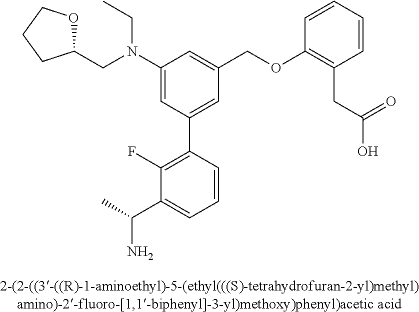 [SMILES: CCN(C[C@@H]1CCCO1)c1cc(COc2ccccc2CC(=O)O)cc(-c2cccc([C@@H](C)N)c2F)c1] | (400 MHz, Methanol-d₄) δ ppm 7.49 (td, J = 7.6, 1.8 Hz, 1H), 7.42 (td, J = 7.2, 6.7, 1.7 Hz, 1H), 7.29 (t, J = 7.7 Hz, 1H), 7.20 (dd, J = 7.4, 1.7 Hz, 1H), 7.13 (ddd, J = 8.2, 7.4, 1.7 Hz, 1H), 7.05 (d, J = 2.2 Hz, 1H), 6.93 (dd, J = 8.3, 1.1 Hz, 1H), 6.90-6.73 (m, 3H), 5.11 (s, 2H), 4.70 (q, J = 6.9 Hz, 1H), 4.14 (qd, J = 6.8, 4.3 Hz, 1H), 3.87 (dt, J = 8.3, 6.8 Hz, 1H), 3.74 (td, J = 7.9, 5.9 Hz, 1H), 3.58 (s, 2H), 3.55- 3.40 (m, 4H), 2.10-1.82 (m, 3H), 1.72-1.60 (m,4H), 1.16 (t, J = 7.0 Hz, 3H). | calcd. for C₃₀H₃₅FN₂O₄ (M + H)⁺ 507.2653 found 507.2649. |
| 216-K | 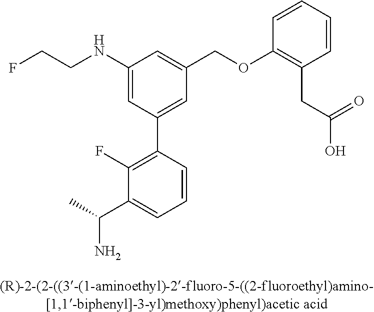 [SMILES: C[C@@H](N)c1cccc(-c2cc(COc3ccccc3CC(=O)O)cc(NCCF)c2)c1F] | (400 MHz, Methanol-d₄) δ ppm 7.45 (dtd, J = 31.4, 7.1, 6.7, 1.8 Hz, 2H), 7.37-7.02 (m, 4H), 7.01-6.77 (m, 3H), 6.71 (d, J = 2.0 Hz, 1H), 5.08 (s, 2H), 4.74-4.61 (m, 2H), 4.53 (t, J = 5.0, 5.0 Hz, 1H), 3.58 (s, 2H), 3.46 (dt, J = 25.9, 5.1, 5.1 Hz, 2H), 1.64 (d, J = 6.9 Hz, 3H). | calcd. for C₂₅H₂₆F₂N₂O₃ (M + H)⁺ 441.1990, found 441.2004. |
| 216-L | 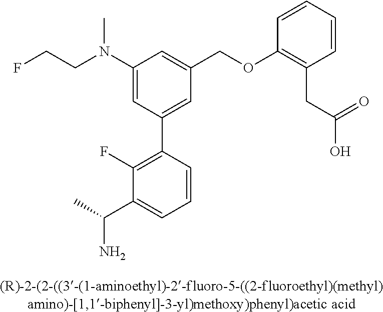 [SMILES: C[C@@H](N)c1cccc(-c2cc(COc3ccccc3CC(=O)O)cc(N(C)CCF)c2)c1F] | (400 MHz, Methanol-d₄) δ ppm 7.50 (td, J = 7.6, 1.8 Hz, 1H), 7.41 (ddd, J = 8.2, 6.7, 1.8 Hz, 1H), 7.27 (t, J = 7.7 Hz, 1H), 7.19 (dd, J = 7.4, 1.7 Hz, 1H), 7.17-7.06 (m, 2H), 6.94 (dd, J = 8.3, 1.1 Hz, 1H), 6.89-6.83 (m, 2H), 6.77 (p, J = 1.1 Hz, 1H), 5.11 (s, 2H), 4.74-4.64 (m, 2H), 4.54 (t, J = 4.9 Hz, 1H), 3.74 (t, J = 5.0 Hz, 1H), 3.68 (t, J = 5.0 Hz, 1H), 3.58 (s, 2H), 3.04 (d, J = 0.8 Hz, 3H), 1.64 (d, J = 6.9 Hz. 3H). | calcd. for C₂₆H₂₈F₂N₂O₃ (M + H)⁺ 455.2141, found 455.2115. |
| 216-M | 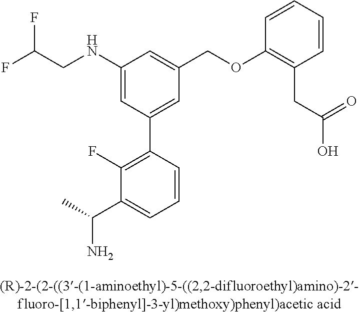 [SMILES: C[C@@H](N)c1cccc(-c2cc(COc3ccccc3CC(=O)O)cc(NCC(F)F)c2)c1F] | (400 MHz, Methanol-d₄) δ ppm 7.49 (td, J = 7.6, 1.8 Hz, 1H), 7.41 (ddd, J = 8.2, 6.7, 1.8 Hz, 1H), 7.27 (t, J = 7.7 Hz,1H), 7.19(dd, J = 7.4, 1.7 Hz, 1H), 7.13 (ddd, J = 8.2, 7.4, 1.7 Hz, 1H), 7.08 (dt, J = 2.6, 1.4 Hz, 1H), 6.93 (dd, J = 8.2, 1.1 Hz, 1H), 6.90-6.80 (m, 2H), 6.74 (dt, J = 2.4, 1.2 Hz, 1H), 5.96 (tt, J = 56.3, 4.1 Hz, 1H), 5.08 (s, 2H), 4.69 (q, J = 6.9 Hz, 1H), 3.63-3.49 (m, 4H), 1.64 (d, J = 6.9 Hz, 3H). | calcd. for C₂₅H₂₅F₃N₂O₃ (M + H)⁺ 459.1890, found 459.1866. |
| 216-N | 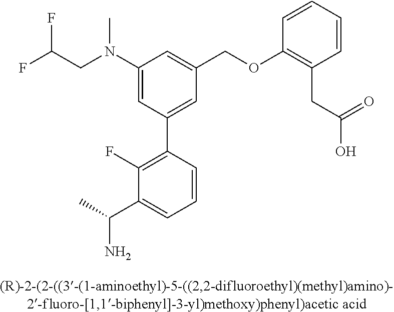 [SMILES: C[C@@H](N)c1cccc(-c2cc(COc3ccccc3CC(=O)O)cc(N(C)CC(F)F)c2)c1F] | (400 MHz, Methanol-d₄) δ ppm 7.53 (td, J = 7.6, 1.7 Hz, 1H), 7.47-7.40 (m, 1H), 7.30 (t, J = 7.7 Hz, 1H), 7.19 (dt, J = 4.5, 1.9 Hz, 2H), 7.14 (td, J = 7.8, 1.8 Hz, 1H), 6.97- 6.90 (m, 2H), 6.89-6.81 (m, 2H), 6.04 (tt, J = 55.8, 4.0 Hz, 1H), 5.13 (s, 2H), 4.71 (q, J = 6.9 Hz, 1H), 3.77 (td, J = 14.8, 4.0 Hz, 2H), 3.58 (s, 2H), 3.09 (s, 3H), 1.67 (d, J = 6.9 Hz, 3H). | calcd. for C₂₆H₂₇F₃N₂O₃ (M + H)⁺ 473.2052, found 473.2032. |
| 216-O | 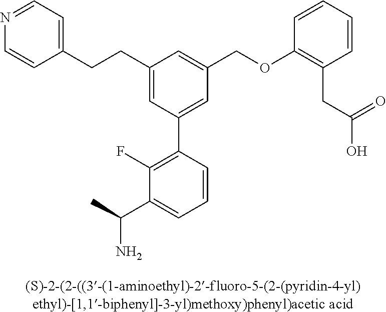 [SMILES: C[C@H](N)c1cccc(-c2cc(CCc3ccncc3)cc(COc3ccccc3CC(=O)O)c2)c1F] | (400 MHz, Methanol-d₄) δ ppm 8.41-8.34 (m, 2H), 7.62 (dd, J = 2.6, 1.5 Hz, 1H), 7.47-7.38 (m, 2H), 7.33- 7.10 (m,7H), 6.95-6.83 (m, 2H), 5.13 (s, 2H), 4.68 (q, J = 6.9 Hz, 1H), 3.58 (s, 2H), 3.03 (s, 4H), 1.63 (d, J = 6.9 Hz, 3H). | calcd. for C₃₀H₂₉FN₂O₃ (M + H)⁺ 485.2235, found 485.2204. |
| 216-P | 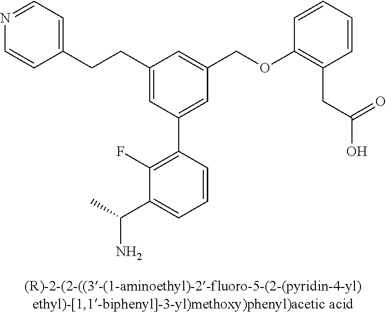 [SMILES: C[C@@H](N)c1cccc(-c2cc(CCc3ccncc3)cc(COc3ccccc3CC(=O)O)c2)c1F] | (400 MHz, Methanol-d₄) δ ppm 8.41-8.34 (m, 2H), 7.62 (dd, J = 2.6, 1.5 Hz, 1H), 7.47-7.38 (m, 2H), 7.33- 7.10 (m, 7H), 6.95-6.83 (m, 2H), 5.13 (s, 2H), 4.68 (q, J = 6.9 Hz, 1H), 3.58 (s, 2H), 3.03 (s, 4H), 1.63 (d, J = 6.9 Hz, 3H). | calcd. for C₃₀H₂₉FN₂O₃ (M + H)⁺ 485.2235, found 485.2191. |
| 216-Q | 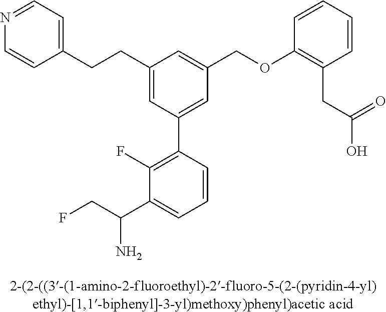 [SMILES: NC(CF)c1cccc(-c2cc(CCc3ccncc3)cc(COc3ccccc3CC(=O)O)c2)c1F] | (400 MHz, Methanol-d₄) δ ppm 8.41-8.33 (m, 2H), 7.53 (q, J = 1.7 Hz, 1H), 7.44 (dtd, J = 19.1, 7.8, 1.8 Hz, 2H), 7.36 (t, J = 1.6 Hz, 1H), 7.31-7.15 (m, 6H), 6.96 (dd, J = 8.2, 1.1 Hz, 1H), 6.89 (td, J = 7.4, 1.1 Hz, 1H), 5.15 (s, 2H), 4.78-4.67 (m, 2H), 4.67-4.57 (m, 1H), 3.62 (s, 2H), 3.04 (s, 4H). | calcd. for C₃₀H₂₈F₂N₂O₃ (M + H)⁺ 503.2140, found 503.2100. |
| 216-R | 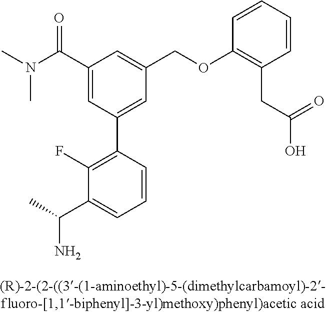 [SMILES: C[C@@H](N)c1cccc(-c2cc(COc3ccccc3CC(=O)O)cc(C(=O)N(C)C)c2)c1F] | (400 MHz, Methanol-d₄) δ ppm 8.00 (s, 1 H), 7.55-7.60 (m, 1 H), 7.51 (m, 3 H), 7.35 (t, J = 7.58 Hz, 1H), 7.12- 7.21 (m, 2 H), 6.95 (d, J = 7.45 Hz, 1 H), 6.88 (t, J = 7.33 Hz, 1 H), 5.24 (s, 2 H), 4.75 (d, J = 7.20 Hz, 1 H), 3.58 (s, 2 H), 3.09-3.13 (m, 3 H), 3.05 (s, 3H), 1.69 (d, J = 6.95 Hz, 3 H). | calcd. for C₂₆H₂₇FN₂O₄ (M + H)⁺ 451.2033, found 451.2000. |
| 216-S | 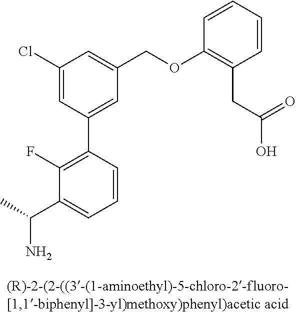 [SMILES: C[C@@H](N)c1cccc(-c2cc(Cl)cc(COc3ccccc3CC(=O)O)c2)c1F] | (400 MHz, DMSO-d₆) δ ppm 7.65 (q, J = 1.5 Hz, 1H), 7.62- 7.56 (m, 1H), 7.53 (dt, J = 6.0, 1.8 Hz, 2H), 7.41 (td, J = 7.6, 1.8 Hz, 1H), 7.27 (t, J = 7.7 Hz, 1H), 7.24-7.17 (m, 2H), 7.00 (dd, J = 8.7, 1.1 Hz, 1H), 6.89 (td, J = 7.4, 1.1 Hz, 1H), 5-22 (s, 2H), 4.35 (q, J = 6.6 Hz, 1H), 3.54 (d, J = 1.2 Hz, 2H), 1.34 (d, J = 6.6 Hz, 3H). | calcd. for C₂₃H₂₁ClFNO₃ (M + H)⁺ 414.1272, found 414.1260. |
| 216-T | 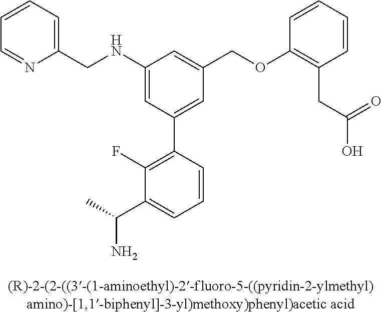 [SMILES: C[C@@H](N)c1cccc(-c2cc(COc3ccccc3CC(=O)O)cc(NCc3ccccn3)c2)c1F] | (400 MHz, Methanol-d₄) δ 8.51-8.44 (m, 1H), 7.76 (td, J = 8.1, 7.9, 2.0 Hz, 1H), 7.51 (d, J = 7.7 Hz, 1H), 7.39 (t, J = 7.5, 7.5 Hz, 2H), 7.31- 7.07 (m, 4H), 7.03 (s, 1H), 6.94-6.80 (m, 2H), 6.76 (dd, J = 2.3, 1.3 Hz, 1H), 6.60 (d, J = 2.1 Hz, 1H), 5.03 (s, 2H), 4.67 (q, J = 6.8, 6.8, 6.7 Hz, 1H), 4.49 (s, 2H), 3.55 (s, 2H), 1.62 (t, J = 6.3, 6.3 Hz, 3H). | calcd. for C₂₉H₂₈FN₃O₃ (M + H)⁺ 486.2193, found 486.2198. |
| 216-U | 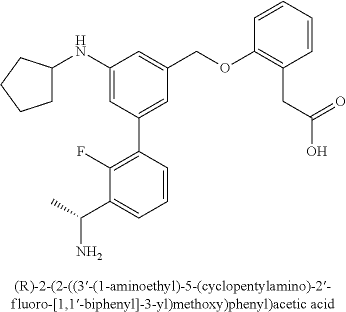 [SMILES: C[C@@H](N)c1cccc(-c2cc(COc3ccccc3CC(=O)O)cc(NC3CCCC3)c2)c1F] | (400 MHz, Methanol-d₄) δ ppm 7.49-7.38 (m, 2H), 7.26 (t, J = 7.7, 7.7 Hz, 1H), 7.20 (dd, J = 7.5, 1.7 Hz, 1H), 7.15- 7.09 (m, 1H), 6.99 (d, J = 2.1 Hz, 1H), 6.92 (dd, J = 8.2, 1.1 Hz, 1H), 6.85 (td, J = 7.4, 7.4, 1.1 Hz, 1H), 6.76 (t, J = 1.7, 1.7 Hz, 1H), 6.68 (d, J = 2.0 Hz, 1H), 5.07 (s, 2H), 4.64 (q, J = 6.9, 6.8, 6.8 Hz, 1H), 3.82 (p, J = 6.2, 6.2, 6.2, 6.2 Hz, 2H), 3.58 (s, 2H), 2.96 (d, J = 11.2 Hz, 1H), 2.01 (dt, J = 12.9, 6.5, 6.5 Hz, 2H), 1.80-1.71 (m, 2H), 1.62 (d, J = 6.9 Hz, 3H), 1.54 (dt, J = 12.8. 6.6, 6.6 Hz, 2H). | calcd. for C₂₈H₃₁FN₂O₃ (M + H)⁺ 463.2397, found 463.2376. |
| 216-V | 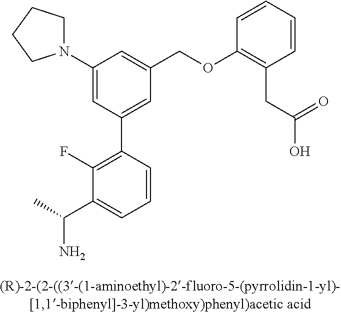 [SMILES: C[C@@H](N)c1cccc(-c2cc(COc3ccccc3CC(=O)O)cc(N3CCCC3)c2)c1F] | (400 MHz, Methanol-d₄) δ ppm 7.53-7.36 (m, 2H), 7.31- 7.08 (m, 3H), 7.04-6.91 (m, 2H), 6.85 (td, J = 7.4, 7.4, 1.1 Hz, 1H), 6.68 (dd, J = 2.3, 1.3 Hz, 1H), 6.59 (dt, J = 2.4, 1.2, 1.2 Hz, 1H), 5.09 (s, 2H), 4.64 (q, J = 6.9, 6.9, 6.9 Hz, 1H), 3.58 (s, 2H), 3.32 (s, 2H), 2.95 (d, J = 9.6 Hz, 2H), 2.09-1.97 (m,4H), 1.61 (d, J = 6.8 Hz, 3H). | calcd. for C₂₇H₂₉FN₂O₃ (M + H)⁺ 449.2240, found 449.2227. |
| 216-W | 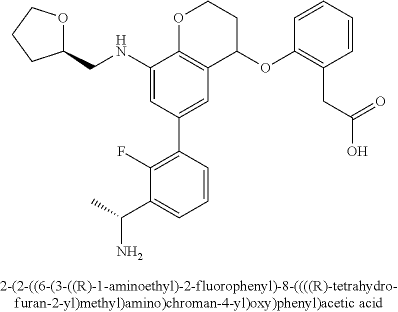 [SMILES: C[C@@H](N)c1cccc(-c2cc(NC[C@H]3CCCO3)c3c(c2)C(Oc2ccccc2CC(=O)O)CCO3)c1F] | (400 MHz, , Methanol-d₄) δ ppm 7.44 (tdd, J = 7.8, 6.4, 1.8 Hz, 1H), 7.39-7.32 (m, 1H), 7.29-7.14 (m, 3H), 7.14-7.07 (m, 1H), 6.95- 6.84 (m, 2H), 6.73 (q, J = 1.9 Hz, 1H), 5.43 (t, J = 4.4 Hz, 1H), 4.67 (dq, J = 17.2, 6.9 Hz, 1H), 4.50-4.34 (m, 2H), 4.21-4.10 (m, 1H), 3.94- 3.85 (m, 1H), 3.78 (td, J = 7.7, 6.4 Hz, 1H), 3.60-3.40 (m, 2H), 3.21-3.09 (m, 1H), 2.37-2.24 (m, 2H), 2.12- 1.84 (m, 3H), 1.78-1.67 (m, 1H), 1.64 (dd, J = 6.9. 1.1 Hz, 3H), 1H overlap with solvent signal. | calcd. for C₃₀H₃₃FN₂O₅ (M + H)⁺ 521.2446, found 521.2415. |
| 216-X | 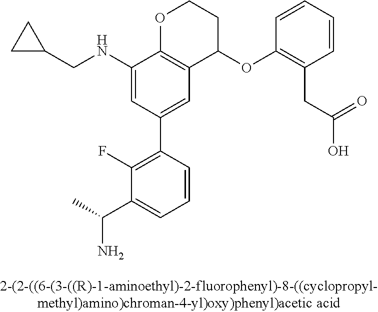 [SMILES: C[C@@H](N)c1cccc(-c2cc(NCC3CC3)c3c(c2)C(Oc2ccccc2CC(=O)O)CCO3)c1F] | (400 MHz, , Methanol-d₄) δ ppm 7.43 (tdd, J = 7.5, 5.1, 1.8 Hz, 1H), 7.40-7.31 (m, 1H), 7.30-7.09 (m, 4H), 6.96-6.82 (m, 2H), 6.69 (s, 1H), 5.43 (d, J = 5.0 Hz, 1H), 4.68 (dq, J = 16.2, 6.9 Hz, 1H), 4.51-4.33 (m, 2H), 3.61-3.41 (m, 2H), 3.01 (dt, J = 6.7, 2.0 Hz, 2H), 2.29 (dt, J = 6.4, 4.6 Hz, 2H), 1.64 (d, J = 6.9 Hz, 3H), 1.22-1.06 (m, 1H), 0.62-0.50 (m, 2H), 0.34-0.22 (m, 2H). | calcd. for C₂₉H₃₁FN₂O₄ (M + H)⁺ 491.2340, found 491.2313. |
| 216-Y | 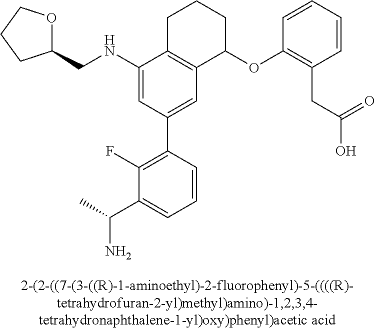 [SMILES: C[C@@H](N)c1cccc(-c2cc(NC[C@H]3CCCO3)c3c(c2)C(Oc2ccccc2CC(=O)O)CCC3)c1F] | (400 MHz, , Methanol-d₄) δ ppm δ 7.47 (dtd, J = 12.2, 7.6, 1.8 Hz, 1H), 7.43-7.34 (m, 1H), 7.29-7.10 (m, 4H), 7.05 (d, J = 8.0 Hz, 1H), 6.87 (tt, J = 7.4, 1.2 Hz, 1H), 6.71 (dd, J = 7.5, 1.6 Hz, 1H), 5.38 (dd, J = 7.0, 3.7 Hz, 1H), 4.68 (dq, J = 28.9, 6.9 Hz, 1H), 4.19 (qd, J = 6.9, 4.5 Hz, 1H), 3.96-3.86 (m, 1H), 3.84- 3.74 (m, 1H), 3.69 (dd, J = 16.0, 9.7 Hz, 1H), 3.43-3.35 (m, 1H), 3.25-3.17 (m, 1H), 2.67-2.43 (m, 2H), 2.26- 1.84 (m, 7H), 1.81-1.69 (m, 1H), 1.65 (dd, J = 7.0, 1.7 Hz, 3H), 1H overlap with solvent signal. | calcd. for C₃₁H₃₅FN₂O₄ (M + H)⁺ 519.2653, found 519.2632. |
| 216-Z | 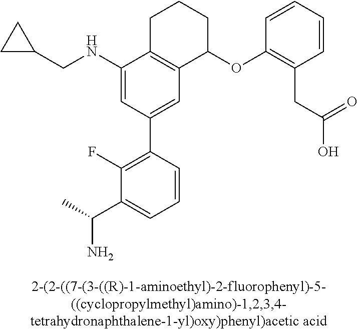 [SMILES: C[C@@H](N)c1cccc(-c2cc(NCC3CC3)c3c(c2)C(Oc2ccccc2CC(=O)O)CCC3)c1F] | (400 MHz, , Methanol-d₄) δ ppm 7.45 (dtd, J = 10.8, 7.6, 1.8 Hz, 1H), 7.41-7.33 (m, 1H), 7.29-7.12 (m, 3H), 7.10-7.02 (m, 2H), 6.87 (tt, J = 7.4, 1.1 Hz, 1H), 6.71- 6.65 (m, 1H), 5.38 (dd, J = 7.2, 3.4 Hz, 1H), 4.67 (dq, J = 27.0, 6.9 Hz, 1H), 3.67 (66, J = 16.0, 9.9 Hz, 1H), 3.40 (dd, J = 16.1, 9.0 Hz, 1H), 3.06 (dd, J = 6.6, 3.4 Hz, 2H), 2.67- 2.45 (m, 2H), 2.18 (tq, J = 8.0, 3.8 Hz, 2H), 2.08-1.84 (m, 2H), 1.64 (dd, J = 7.0, 1.6 Hz, 3H), 1.18 (tttd, J = 8.1, 6.5, 4.8, 1.4 Hz, 1H), 0.55 (dddd, J = 7.9, 5.7, 4.3, 1.3 Hz, 2H), 0.28 (dtd, J = 6.0, 4.4, 1.6 Hz, 2H). | calcd. for C₃₀H₃₃FN₂O₃ (M + H)⁺ 489.2548, found 489.2523. |
| 216-AA | 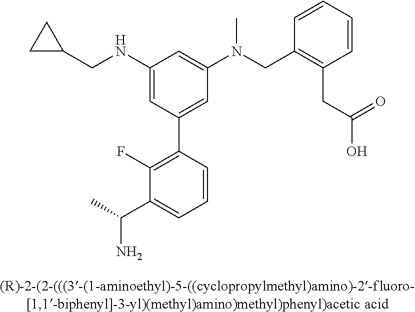 [SMILES: C[C@@H](N)c1cccc(-c2cc(NCC3CC3)cc(N(C)Cc3ccccc3CC(=O)O)c2)c1F] | (400 MHz, , Methanol-d₄) δ ppm 7.42 (td, J = 7.5, 1.8 Hz, 1H), 7.35 (ddd, J = 8.2, 6.7, 1.8 Hz, 1H), 7.25-7.17 (m, 2H), 7.14-7.00 (m, 3H), 6.25 (td, J = 2.4, 1.3 Hz, 1H), 6.19-6.12 (m, 2H), 4.73- 4.54 (m, 3H), 3.55 (d, J = 1.9 Hz, 2H), 3.07 (s, 3H), 2.95 (d, J = 6.7 Hz, 2H), 1.64 (d, J = 6.9 Hz, 3H), 1.08 (ttt, J = 8.1, 6.7, 4.8 Hz, 1H), 0.57-0.47 (m, 2H), 0.27-0.20 (m, 2H). | calcd. for C₂₈H₃₂FN₃O₂ (M + H)⁺ 462.2551, found 462.2519. |
| 216-AB | 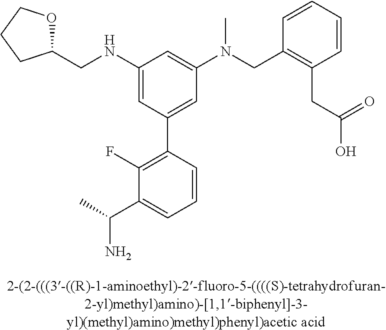 [SMILES: C[C@@H](N)c1cccc(-c2cc(NC[C@@H]3CCCO3)cc(N(C)Cc3ccccc3CC(=O)O)c2)c1F] | (400 MHz, Methanol-d₄) δ ppm 7.41 (td, J = 7.5, 1.8 Hz, 1H), 7.37 (s, 1H), 7.24-7.16 (m, 2H), 7.07 (dddd, J = 22.3, 13.5, 7.3, 1.9 Hz, 3H), 6.24 (q, J = 2.0 Hz, 1H), 6.14 (d, J = 1.4 Hz, 2H), 4.70-4.58 (m, 3H), 4.09 (qd, J = 6.8, 4.8 Hz, 1H), 3.87 (ddd, J = 8.3, 7.0, 6.2 Hz, 1H), 3.75 (td, J = 7.8, 6.3 Hz, 1H), 3.55 (d, J = 2.0 Hz, 2H), 3.25-3.10 (m, 2H), 3.07 (s, 3H), 2.08-1.83 (m,3H), 1.73-1.59 (m, 4H). | calcd. for C₂₉H₃₄FN₃O₃ (M + H)⁺ 492.2657, found 492.2672. |
| 216-AC | 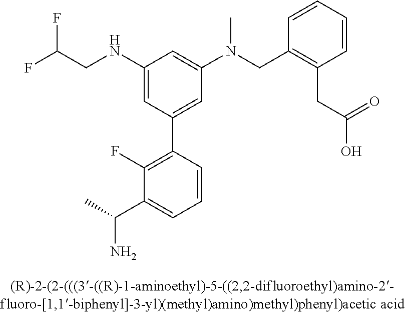 [SMILES: C[C@@H](N)c1cccc(-c2cc(NCC(F)F)cc(N(C)Cc3ccccc3CC(=O)O)c2)c1F] | (400 MHz, Methanol-d₄) δ ppm 7.41 (td, J = 7.5, 1.8 Hz, 1H), 7.36 (ddd, J = 8.2, 6.8, 1.8 Hz, 1H), 7.24-7.17 (m, 2H), 7.15-6.99 (m, 3H), 6.27 (q, J = 2.0 Hz, 1H), 6.14 (d, J = 1.8 Hz, 2H), 5.92 (tt, J = 56.4, 4.2 Hz, 1H), 4.71- 4.55 (m, 3H), 3.56-3.44 (m, 4H), 3.08 (s, 3H), 1.62 (d, J = 6.9 Hz, 3H). | calcd. for C₂₆H₂₈F₃N₃O₂ (M + H)⁺ 472.2206, found 472.2215 |
| 216-AD | 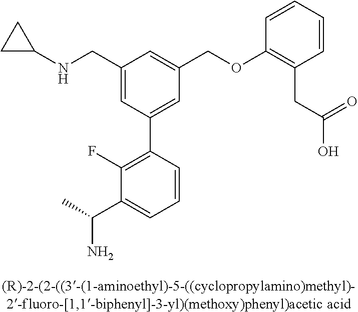 [SMILES: C[C@@H](N)c1cccc(-c2cc(CNC3CC3)cc(COc3ccccc3CC(=O)O)c2)c1F] | (400 MHz, Methanol-d₄) δ ppm 7.70 (d, J = 2.3 Hz, 1H), 7.62 (t, J = 1.6 Hz, 1H), 7.54- 7.44 (m, 3H), 7.30 (t, J = 7.7 Hz, 1H), 7.21-7.09 (m, 2H), 6.95 (dd, J = 8.3, 1.1 Hz, 1H), 6.86 (td, J = 7.4, 1.1 Hz, 1H), 5.20 (s, 2H), 4.62 (q, J = 6.9 Hz, 1H), 4.05 (s, 2H), 3.54 (s, 2H), 2.43-2.33 (m, 1H), 1.60 (d, J = 6.8 Hz, 3H), 0.65-0.57 (m, 4H). | calcd. for C₂₇H₂₉FN₂O₃ (M + H)⁺ 449.2235, found 449.2207. |
| 216-AE | 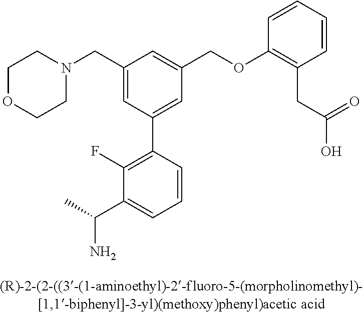 [SMILES: C[C@@H](N)c1cccc(-c2cc(COc3ccccc3CC(=O)O)cc(CN3CCOCC3)c2)c1F] | (400 MHz, Methanol-d₄) δ ppm 7.82-7.75 (m, 1H), 7.53 (td, J = 7.6, 1.8 Hz, 1H), 7.45 (dq, J = 3.3, 1.8 Hz, 3H), 7.32 (t, J = 7.7 Hz, 1H), 7.21- 7.10 (m, 2H), 6.95 (dd, J = 8.3, 1.1 Hz, 1H), 6.90-6.84 (m, 1H), 5.19 (d, J = 2.6 Hz, 2H), 4.71 (q, J = 6.9 Hz, 1H), 3.71 (q, J = 4.9 Hz, 4H), 3.66 (s, 2H), 3.57 (d, J = 3.9 Hz, 2H), 2.61-2.49 (m, 4H), 1.66 (d, J = 6.9 Hz, 3H). | calcd. for C₂₈H₃₁FN₂O₄ (M + H)⁺ 479.2340, found 479.2320. |
| 216-AF | 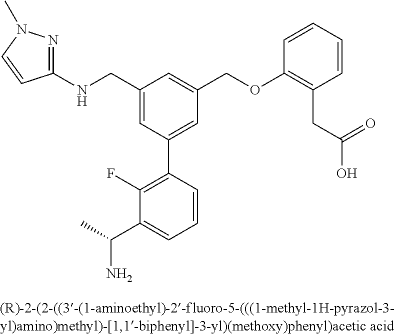 [SMILES: C[C@@H](N)c1cccc(-c2cc(CNc3ccn(C)n3)cc(COc3ccccc3CC(=O)O)c2)c1F] | (400 MHz, Methanol-d₄) δ ppm 7.73 (d, J = 2.5 Hz, 1H), 7.55-7.40 (m, 4H), 7.29 (t, J = 7.7 Hz, 1H), 7.26-7.17 (m, 2H), 7.17-7.09 (m, 1H), 6.93 (dd, J = 8.2, 1.1 Hz, 1H), 6.86 (td, J = 7.4, 1.1 Hz, 1H), 5.55 (d, J = 2.3 Hz, 1H), 5.16 (s, 2H), 4.71 (q, J = 6.9 Hz, 1H), 4.38 (s, 2H), 3.66 (s, 3H), 3.57 (s, 2H), 1.66 (d, J = 6.9 Hz, 3H). | calcd. for C₂₈H₂₉FN₄O₃ (M + H)⁺ 489.2296, found 489.2275. |
| 216-AG | 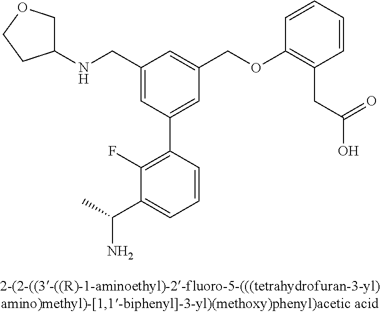 [SMILES: C[C@@H](N)c1cccc(-c2cc(CNC3CCOC3)cc(COc3ccccc3CC(=O)O)c2)c1F] | (400 MHz, Methanol-d₄) δ ppm 7.68 (d, J = 14.9 Hz, 2H), 7.48 (qd, J = 6.8, 6.0, 1.7 Hz, 3H), 7.30 (t, J = 7.7 Hz, 1H), 7.21-7.10 (m, 2H), 6.95 (dd, J = 8.2, 1.1 Hz, 1H), 6.86 (td, J = 7.4, 1.1 Hz, 1H), 5.21 (s, 2H), 4.60 (q, J = 6.9 Hz, 1H), 4.06-3.92 (m, 3H), 3.87-3.69 (m, 3H), 3.66- 3.52 (m, 3H), 2.20 (dtd, J = 13.7, 7.9, 6.0 Hz, 1H), 2.01- 1.91 (m, 1H), 1.59 (d, J = 6.8 Hz, 3H). | calcd. for C₂₈H₃₁FN₂O₄ (M + H)⁺ 479.2340, found 479.2313. |
| 216-AH | 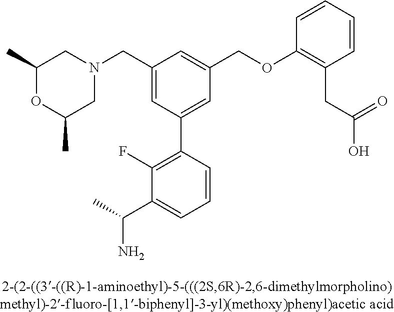 [SMILES: C[C@@H]1CN(Cc2cc(COc3ccccc3CC(=O)O)cc(-c3cccc([C@@H](C)N)c3F)c2)C[C@H](C)O1] | (400 MHz, Methanol-d₄) δ ppm 7.79 (d, J = 2.3 Hz, 1H), 7.53 (td, J = 7.6, 1.7 Hz, 1H), 7.50-7.41 (m, 3H), 7.32 (t, J = 7.7 Hz, 1H), 7.19 (dd, J = 7.4, 1.7 Hz, 1H), 7.14 (ddd, J = 9.2, 7.5, 1.8 Hz, 1H), 6.95 (dd, J = 8.2, 1.1 Hz, 1H), 6.86 (td, J = 7.4, 1.1 Hz, 1H), 5.19 (s, 2H), 4.71 (q, J = 6.9 Hz, 1H), 3.70 (ddt, J = 12.9, 6.6, 3.2 Hz, 2H), 3.65 (s, 2H), 3.57 (s, 2H), 2.82 (dt, J = 10.7, 1.7 Hz, 2H), 1.85 (dd, J = 11.6, 10.2 Hz, 2H), 1.66 (d, J = 6.9 Hz, 3H), 1.11 (d, J = 6.2 Hz, 6H). | calcd. for C₃₀H₃₅FN₂O₄ (M + H)⁺ 507.2653, found 507.2639. |
| 216-AI | 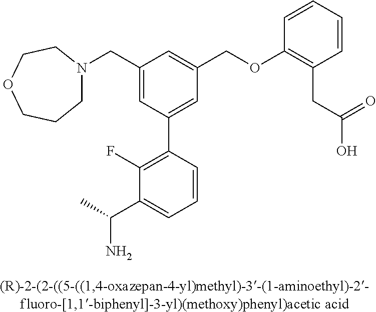 [SMILES: C[C@@H](N)c1cccc(-c2cc(COc3ccccc3CC(=O)O)cc(CN3CCCOCC3)c2)c1F] | (400 MHz, Methanol-d₄) δ ppm 7.72 (s, 1H), 7.57 (t, J = 1.6 Hz, 1H), 7.48 (dtd, J = 10.4, 8.5, 8.0, 1.8 Hz, 3H), 7.30 (t, J = 7.7 Hz, 1H), 7.23- 7.11 (m, 2H), 6.95 (dd, J = 8.1, 1.1 Hz, 1H), 6.86 (td, J = 7.4, 1.1 Hz, 1H), 5.20 (s, 2H), 4.63 (q, J = 6.8 Hz, 1H), 3.91 (s, 2H), 3.85-3.72 (m, 4H), 3.56 (s, 2H), 2.95-2.88 (m, 2H), 2.88-2.82 (m, 2H), 2.03-1.93 (m, 2H), 1.60 (d, J = 6.8 Hz, 3H). | calcd. for C₂₉H₃₃FN₂O₄ (M + H)⁺ 493.2497, found 493.2482. |
| 216-AJ | 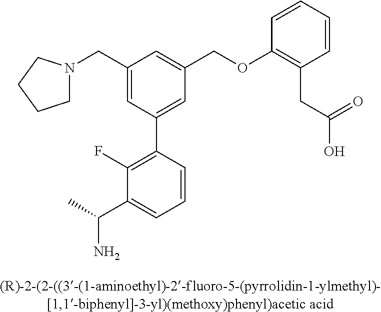 [SMILES: C[C@@H](N)c1cccc(-c2cc(COc3ccccc3CC(=O)O)cc(CN3CCCC3)c2)c1F] | (400 MHz, Methanol-d₄) δ ppm 7.83 (t, J = 1.5, 1.5 Hz, 1H), 7.62 (q, J = 1.6, 1.5, 1.5 Hz, 1H), 7.55-7.37 (m,3H), 7.28 (t, J = 7.7, 7.7 Hz, 1H), 7.21-7.11 (m, 2H), 6.97 (dd, J = 8.1, 1.1 Hz, 1H), 6.87 (td, J = 7.4, 7.4, 1.1 Hz, 1H), 5.22 (s, 2H), 4.48 (q, J = 6.7, 6.7, 6.7 Hz, 1H), 4.19 (s, 2H), 3.52 (s, 2H), 3.11-3.05 (m, 4H), 2.02-1.96 (m, 4H), 1.50 (d, J = 6.7 Hz, 3H). | calcd. for C₂₈H₃₁FN₂O₃ (M + H)⁺ 463.2397, found 463.2413. |

### Example 217

### Example 217-A

### Tert-Butyl 2-(2-((3-bromo-5-(morpholinomethyl)benzyl)oxy)phenyl)acetate

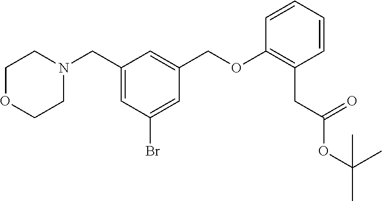 [SMILES: CC(C)(C)OC(=O)Cc1ccccc1OCc1cc(Br)cc(CN2CCOCC2)c1]

A solution of tert-butyl 2-(2-((3-bromo-5-(hydroxymethyl)benzyl)oxy)phenyl)acetate (Intermediate 59A) (2000 mg, 4.91 mmol) and methanesulfonyl chloride (562 mg, 4.91 mmol) in THF (20 mL) was cooled in an ice/water bath. TEA (1.369 mL, 9.82 mmol) was added drop wise. The resulting solution was allowed to warm to room temperature. 2 hours later, morpholine (1.283 mL, 14.73 mmol) was added and the mixture was stirred at room temperature for 16 hours. The mixture was quenched with water, extracted with EtOAc, dried with MgSO₄, filtered and concentrated. This was purified by flash chromatography (0-60% EtOAc:Heptane) to provide title compound. MS (ESI+) m/z: 476.1, 478.1 (M+H).

### Example 217-B

### tert-Butyl 2-(2-((3-(morpholinomethyl)-5-(4,4,5,5-tetramethyl-1,3,2-dioxaborolan-2-yl)benzyl)oxy)phenyl)acetate

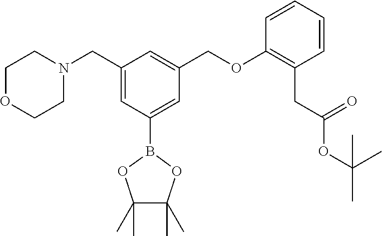 [SMILES: CC(C)(C)OC(=O)Cc1ccccc1OCc1cc(CN2CCOCC2)cc(B2OC(C)(C)C(C)(C)O2)c1]

To a solution of tert-butyl 2-(2-((3-bromo-5-(morpholinomethyl)benzyl)oxy)phenyl)acetate (600 mg, 1.259 mmol) in DMF (10 mL), 4,4,4′,4′,5,5,5′,5′-octamethyl-2,2′-bi(1,3,2-dioxaborolane) (640 mg, 2.52 mmol), potassium acetate (371 mg, 3.78 mmol), PdCl₂(dppf).CH₂Cl₂ adduct (CAS #95464-05-4) (51.4 mg, 0.063 mmol) were added and the reaction heated to 110° C. for 2 hours. The reaction was then cooled to room temperature, poured into water, extracted with EtOAc/Heptane (50%) evaporated and purified using FCC (0-30% EtOAc/Heptane) to provide title product. MS (ESI+) m/z 523.9 (M+H).

### Example 217-C

### (+) or (−)-tert-Butyl 2-(2-((3′-(1-((tert-butoxycarbonyl)amino)-2-fluoroethyl)-2′-fluoro-5-(morpholinomethyl)-[1,1′-biphenyl]-3-yl)methoxy)phenyl)acetate

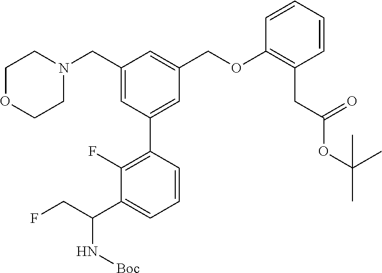 [SMILES: CC(C)(C)OC(=O)Cc1ccccc1OCc1cc(CN2CCOCC2)cc(-c2cccc(C(CF)NC(=O)OC(C)(C)C)c2F)c1]

In a 2-5 mL microwave vial was placed tert-butyl 2-(2-((3-(morpholinomethyl)-5-(4,4,5,5-tetramethyl-1,3,2-dioxaborolan-2-yl)benzyl)oxy)phenyl)acetate (300 mg, 0.430 mmol), tert-butyl (1-(3-bromo-2-fluorophenyl)-2-fluoroethyl)carbamate (Intermediate 33-C2) (159 mg, 0.473 mmol), PdCl₂(dppf).CH₂Cl₂ adduct (CAS #95464-05-4) (15.73 mg, 0.021 mmol) in acetonitrile (3 mL). K₃PO₄ (2M aq. solution, 0.645 ml, 1.289 mmol) was added and the vial was sealed and heated in a microwave at 110° C. for 60 min. The reaction mixture was cooled to rt. The organic layer was filtered and purified by flash chromatography (0-50% EtOAc/Heptane) to give the title compound. MS (ESI+) m/z 653.1 (M+H).

### Example 217-D

### (+) or (−)-2-(2-((3′-(1-Amino-2-fluoroethyl)-2′-fluoro-5-(morpholinomethyl)-[1,1′-biphenyl]-3-yl)methoxy)phenyl)acetic acid

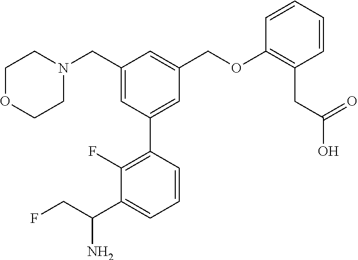 [SMILES: NC(CF)c1cccc(-c2cc(COc3ccccc3CC(=O)O)cc(CN3CCOCC3)c2)c1F]

Tert-butyl 2-(2-((3′-(1-((tert-butoxycarbonyl)amino)-2-fluoroethyl)-2′-fluoro-5-(morpholinomethyl)-[1,1′-biphenyl]-3-yl)methoxy)phenyl)acetate (100 mg, 0.153 mmol) was dissolved in TFA (1.180 mL, 15.32 mmol) and DCM (2 mL). The mixture was stirred at room temperature for 2 hours. It was then concentrated and the residue was purified by prep HPLC (Sunfire C18; 10-70% ACN/H₂O w/0.1% NH₄OH) to give title compound. ¹H NMR (400 MHz, Methanol-d4) δ ppm 7.63 (dt, J=8.2, 1.7 Hz, 2H), 7.54-7.45 (m, 3H), 7.31 (dd, J=8.2, 7.3 Hz, 1H), 7.23-7.13 (m, 2H), 7.02-6.96 (m, 1H), 6.88 (td, J=7.4, 1.1 Hz, 1H), 5.20 (s, 2H), 4.76-4.53 (m, 3H), 3.81 (s, 2H), 3.79 (s, 4H), 3.58 (s, 2H), 2.74-2.65 (m, 4H). HRMS calcd. for C₂₈H₃₀F₂N₂O₄(M+H)⁺ 497.2246. found 497.2227.

### Example 218

### Example 218-A

### Methyl 3-chloro-5-((trimethylsilyl)ethynyl)benzoate

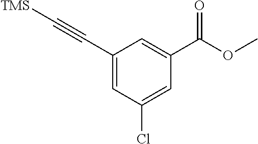 [SMILES: COC(=O)c1cc(Cl)cc(C#C[Si](C)(C)C)c1]

A mixture of methyl 3-chloro-5-iodobenzoate (3 g, 10.1 mmol), Cul (0.193 g, 1.01 mmol), Pd(PPh₃)₄ (0.585 g, 0.506 mmol) in toluene (40 mL) at −78° C. was left on high vacuum for 3 min, then back-flushed with N₂. Ethynyltrimethylsilane (1.71 mL, 12.1 mmol) and diisopropylamine (4.33 mL, 30.4 mmol) were added and the resulting mixture was warmed to rt and stirred overnight. The reaction mixture was partitioned between EtOAc and water. The aqueous layer was extracted with EtOAc. The combined organics were washed with brine, dried (Na₂SO₄) and concentrated. The residue was purified by silica gel chromatography (EtOAc-heptane 0-50%) to provide the desired product as a white solid. ¹H NMR (400 MHz, CD₂Cl₂) δ ppm 7.88 (t, J=1.5 Hz, 1H), 7.84 (dd, J=2.1, 1.5 Hz, 1H), 7.51 (dd, J=2.1, 1.5 Hz, 1H), 3.80 (s, 3H), 0.15 (s, 9H).

### Example 218-B

### (3-Chloro-5-((trimethylsilyl)ethynyl)phenyl)methanol

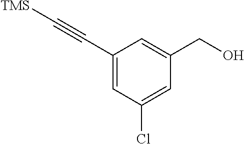 [SMILES: C[Si](C)(C)C#Cc1cc(Cl)cc(CO)c1]

LiBH₄ (0.612 g, 28.1 mmol) was added to a solution of methyl 3-chloro-5-((trimethylsilyl)ethynyl)benzoate (2.5 g, 9.37 mmol) in THF (100 mL) at −78° C., followed by MeOH (1.14 mL, 28.1 mmol). The resulting mixture was allowed to warm to room temperature and stirred for 4.5 hr. LC-MS showed the reaction was completed. The mixture was cooled to 0° C. and sat. NH₄Cl was added slowly to quench excess LiBH₄. The mixture was then extracted with EtOAc (3×). The combined organics were washed with brine, dried (Na₂SO₄) and concentrated. The residue was purified by silica gel chromatography (EtOAc-heptane 0-50%) to provide the desired product as a colorless oil. ¹H NMR (400 MHz, CD₂Cl₂) δ ppm 7.36-7.17 (m, 3H), 4.56 (s, 2H), 0.18 (s, 9H).

### Example 218-C

### Tert-butyl 2-(2-((3-chloro-5-((trimethylsilyl)ethynyl)benzyl)oxy)phenyl)acetate

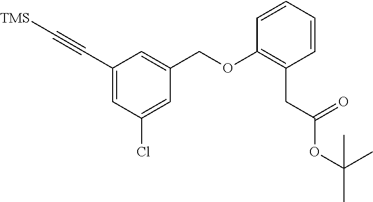 [SMILES: CC(C)(C)OC(=O)Cc1ccccc1OCc1cc(Cl)cc(C#C[Si](C)(C)C)c1]

DIAD (1.88 mL, 9.65 mmol) was added dropwise to a solution of (3-chloro-5-((trimethylsilyl)ethynyl)phenyl)methanol (1.92 g, 8.04 mmol), tert-butyl 2-(2-hydroxyphenyl)acetate (Intermediate 21) (2.01 g, 9.65 mmol) and PPh₃ (2.53 g, 9.65 mmol) in THF (100 mL) at 0° C. The resulting yellow solution was allowed to warm to room temperature and then stirred for 2 hr. Sat. NH₄Cl was added and the mixture was extracted with EtOAc (2×). The combined organics were washed with brine, dried (Na₂SO₄) and concentrated. The residue was purified by silica gel chromatography (EtOAc-heptane 0-50%) to provide the desired product as a yellowish solid. ¹H NMR (400 MHz, CD₂Cl₂) δ ppm 7.42-7.30 (m, 3H), 7.24-7.09 (m, 2H), 6.97-6.86 (m, 1H), 6.83 (dd, J=8.2, 1.1 Hz, 1H), 4.97 (d, J=0.9 Hz, 2H), 3.52 (s, 2H), 1.34 (s, 9H), 0.19 (s, 9H).

### Example 218-D

### Tert-butyl 2-(2-((3-chloro-5-ethynylbenzyl)oxy)phenyl)acetate

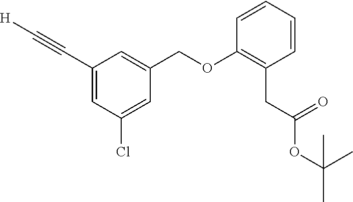 [SMILES: C#Cc1cc(Cl)cc(COc2ccccc2CC(=O)OC(C)(C)C)c1]

K₂CO₃ (0.966 g, 6.99 mmol) was added to a suspension of tert-butyl 2-(2-((3-chloro-5-((trimethylsilyl)ethynyl)benzyl)oxy)phenyl)acetate (3 g, 6.99 mmol) in MeOH (30 mL) at room temperature. The reaction mixture was stirred at room temperature for 1 hr. Solvents were removed and the residue was partitioned between EtOAc and water. The aqueous layer was extracted with EtOAc. The combined organics were washed with brine, dried (Na₂SO₄) and concentrated. The residue was purified by silica gel chromatography (EtOAc-heptane 0-50%) to provide the desired product as a white solid. ¹H NMR (400 MHz, CD₂Cl₂) δ ppm 7.39 (m, 3H), 7.24-7.09 (m, 2H), 6.95-6.87 (m, 1H), 6.87-6.80 (m, 1H), 4.98 (s, 2H), 3.52 (s, 2H), 3.14 (s, 1H), 1.34 (s, 9H).

### Example 218-E

### Tert-butyl 2-(2-((3-chloro-5-((1-methyl-1H-pyrazol-3-yl)ethynyl)benzyl)oxy)phenyl)acetate

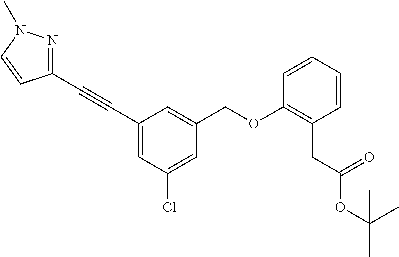 [SMILES: Cn1ccc(C#Cc2cc(Cl)cc(COc3ccccc3CC(=O)OC(C)(C)C)c2)n1]

A mixture of tert-butyl 2-(2-((3-chloro-5-ethynylbenzyl)oxy)phenyl)acetate (300 mg, 0.841 mmol), 3-iodo-1-methyl-1H-pyrazole (350 mg, 1.68 mmol), Cul (16.0 mg, 0.084 mmol) and Pd(PPh₃)₄ (48.6 mg, 0.042 mmol) in toluene (4 mL) at −78° C. was left on high vacuum for 3 min, then back-flushed with N₂. Diisopropylethylamine (0.359 mL, 2.52 mmol) was added and the resulting mixture was warmed to room temperature and stirred overnight. The reaction mixture was partitioned between EtOAc and 1N HCl. The aqueous layer was extracted with EtOAc. The combined organics were washed with brine, dried (Na₂SO₄) and concentrated. The residue was purified by silica gel chromatography (EtOAc-heptane 0-60%) to provide the desired product as a white solid. ¹H NMR (400 MHz, CD₂Cl₂) δ ppm 7.59-7.47 (m, 3H), 7.43 (d, J=2.3 Hz, 1H), 7.33-7.20 (m, 2H), 7.05-6.91 (m, 2H), 6.50 (dd, J=2.3, 0.7 Hz, 1H), 5.10 (s, 2H), 3.96 (d, J=0.7 Hz, 3H), 3.63 (s, 2H), 1.44 (d, J=0.7 Hz, 9H).

### Example 218-F

### (R)-Tert-butyl 2-(2-((3′-(1-((tert-butoxycarbonyl)amino)ethyl)-2′-fluoro-5-((1-methyl-1H-pyrazol-3-yl)ethynyl)-[1,1′-biphenyl]-3-yl)methoxy)phenyl)acetate

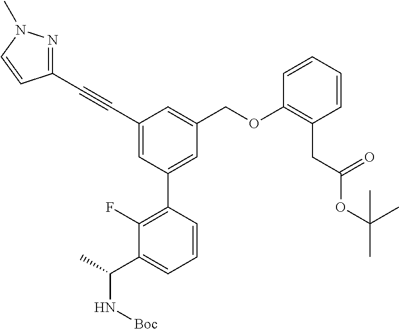 [SMILES: C[C@@H](NC(=O)OC(C)(C)C)c1cccc(-c2cc(C#Cc3ccn(C)n3)cc(COc3ccccc3CC(=O)OC(C)(C)C)c2)c1F]

A mixture of tert-butyl 2-(2-((3-chloro-5-((1-methyl-1H-pyrazol-3-yl)ethynyl)benzyl)oxy)phenyl)acetate (360 mg, 0.824 mmol), (R)-tert-butyl (1-(2-fluoro-3-(4,4,5,5-tetramethyl-1,3,2-dioxaborolan-2-yl)phenyl)ethyl)carbamate (Intermediate 27-B) (331 mg, 0.906 mmol), and 1st generation S-Phos palladacyle (27.7 mg, 0.041 mmol) was left on high vac and back flushed with N₂. DMF (5 mL) was added, followed by K₃PO₄ (2 M) (1.24 ml, 2.47 mmol) was added. The reaction mixture was heated at 110° C. for 1 hr in the microwave then partitioned between EtOAc and sat. NH₄Cl. The aqueous layer was extracted with EtOAc. The combined organics were washed with brine, dried (Na₂SO₄) and concentrated. The residue was purified by silica gel chromatography (EtOAc-heptane 0-60%) to provide the desired product as a yellowish solid. ¹H NMR (400 MHz, CD₂Cl₂) δ ppm 7.73 (d, J=1.7 Hz, 1H), 7.67 (s, 2H), 7.42 (d, J=2.2 Hz, 6H), 7.08-6.94 (m, 2H), 6.50 (d, J=2.3 Hz, 1H), 5.18 (s, 2H), 3.95 (s, 3H), 3.63 (s, 3H), 1.37 (s, 9H), 1.24 (s, 9H).

### Example 218-G

### (R)-2-(2-((3′-(1-Aminoethyl)-2′-fluoro-5-((1-methyl-1H-pyrazol-3-yl)ethynyl)-[1,1′-biphenyl]-3-yl)methoxy)phenyl)acetic acid

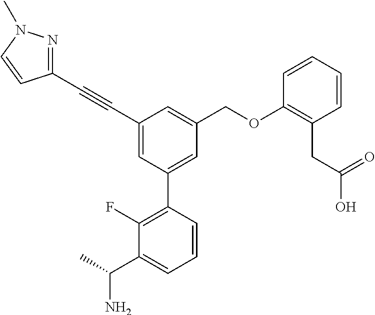 [SMILES: C[C@@H](N)c1cccc(-c2cc(C#Cc3ccn(C)n3)cc(COc3ccccc3CC(=O)O)c2)c1F]

TFA (0.5 mL, 6.49 mmol) was added to a solution of (R)-tert-butyl 2-(2-((3′-(1-((tert-butoxycarbonyl)amino)ethyl)-2′-fluoro-5-((1-methyl-1H-pyrazol-3-yl)ethynyl)-[1,1′-biphenyl]-3-yl)methoxy)phenyl)acetate (80 mg, 0.125 mmol) in DCM (2 mL) at rt. The reaction mixture was stirred at room temperature for 2 hr, and then was concentrated. The residue was purified by reverse phase HPLC (CH₃CN-water (0.1% NH₄OH) to provide the desired product as a white solid. ¹H NMR (400 MHz, DMSO-d₆) δ ppm 7.79 (d, J=2.2 Hz, 1H), 7.73 (d, J=1.7 Hz, 1H), 7.60 (d, J=1.6 Hz, 3H), 7.45 (d, J=1.8 Hz, 1H), 7.20 (d, J=7.3 Hz, 3H), 7.03 (d, J=1.2 Hz, 1H), 6.89 (d, J=1.1 Hz, 1H), 6.54 (d, J=2.3 Hz, 1H), 5.23 (s, 2H), 4.38 (d, J=6.7 Hz, 1H), 3.87 (s, 3H), 3.55 (s, 2H), 1.36 (d, J=6.6 Hz, 3H). HRMS calcd. for C₂₉H₂₆FN₃O₃(M+H)⁺ 484.2036. found 484.1997.

### Example 218-H

### (R)-tert-butyl 2-(2-((3′-(1-((tert-butoxycarbonyl)amino)ethyl)-2′-fluoro-5-(2-(1-methyl-1H-pyrazol-3-yl)ethyl)-[1,1′-biphenyl]-3-yl)methoxy)phenyl)acetate

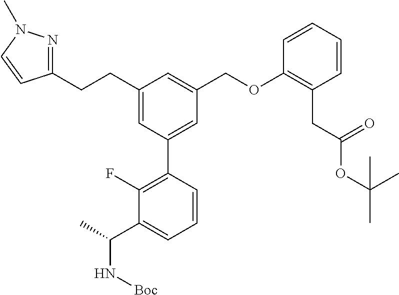 [SMILES: C[C@@H](NC(=O)OC(C)(C)C)c1cccc(-c2cc(CCc3ccn(C)n3)cc(COc3ccccc3CC(=O)OC(C)(C)C)c2)c1F]

A solution of (R)-tert-butyl 2-(2-((3′-(1-((tert-butoxycarbonyl)amino)ethyl)-2′-fluoro-5-((1-methyl-1H-pyrazol-3-yl)ethynyl)-[1,1′-biphenyl]-3-yl)methoxy)phenyl)acetate (150 mg, 0.234 mmol) in MeOH (10 mL) was passed through H-Cube at 1 mL/min at 1 atm of H₂ with Pd/C (10%) as the catalyst for 1 hour. The resulting solution was concentrated to provide the desired product. MS (ESI+) m/z 644.6 (M+1).

### Example 218-1

### (R)-2-(2-((3′-(1-aminoethyl)-2′-fluoro-5-(2-(1-methyl-1H-pyrazol-3-yl)ethyl)-[1,1′-biphenyl]-3-yl)methoxy)phenyl)acetic acid

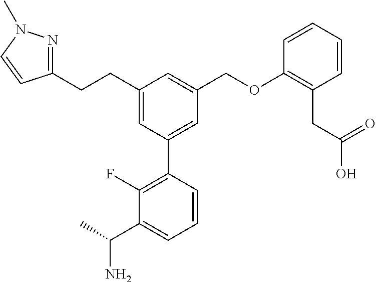 [SMILES: C[C@@H](N)c1cccc(-c2cc(CCc3ccn(C)n3)cc(COc3ccccc3CC(=O)O)c2)c1F]

The title compound were prepared with similar methods as described in Example 216 using (R)-tert-butyl 2-(2-((3′-(1-((tert-butoxycarbonyl)amino)ethyl)-2′-fluoro-5-(2-(1-methyl-1H-pyrazol-3-yl)ethyl)-[1,1′-biphenyl]-3-yl)methoxy)phenyl)acetate. ¹H NMR (400 MHz, Methanol-d₄) δ ppm 7.67 (dt, J=2.8, 1.6 Hz, 1H), 7.53-7.37 (m, 3H), 7.36-7.10 (m, 5H), 7.01-6.82 (m, 2H), 6.06 (d, J=2.2 Hz, 1H), 5.14 (s, 2H), 4.72 (q, J=6.9 Hz, 1H), 3.81 (s, 3H), 3.58 (s, 2H), 3.06-2.90 (m, 4H), 1.67 (d, J=6.9 Hz, 3H). HRMS calcd. for C₂₉H₃₀FN₃O₃(M+H)⁺ 488.2340. found 488.2319.

### Example 219

### Example 219-A

### Methyl 2-(2-((7-bromo-3,4-dihydroquinolin-1(2H)-yl)methyl)phenyl)acetate

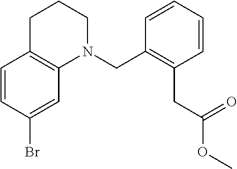 [SMILES: COC(=O)Cc1ccccc1CN1CCCc2ccc(Br)cc21]

To a solution of methyl 2-(2-formylphenyl)acetate (CAS #63969-83-5) (150 mg, 0.842 mmol) and sodium triacetoxyborohydride (214 mg, 1.010 mmol) in DCM (4 ml) at rt was added 7-bromo-1,2,3,4-tetrahydroquinoline hydrochloride (230 mg, 0.926 mmol) (CAS #114744-51-3). The reaction mixture was stirred at rt for 6 hours before sodium triacetoxyborohydride (53.5 mg, 0.25 mmol) was added and the reaction mixture was stirred for 60 hours, then quenched by addition of water. The organic phase was extracted twice with DCM, dried (magnesium sulfate) and evaporated. The residue was purified by flash chromatography on silica gel (eluent heptane/ethyl acetate 100:0 to 70:30) to yield the title compound. MS (ESI+) m/z: 374.1, 376.1 (M+H).

### Example 219-B

### (R)-methyl 2-(2-((7-(3-(1-((tert-butoxycarbonyl)amino)ethyl)-2-fluorophenyl)-3,4-dihydroquinolin-1(2H)-yl)methyl)phenyl)acetate

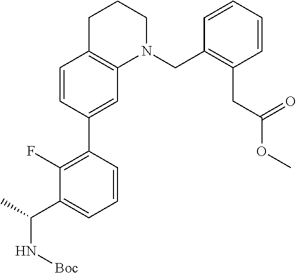 [SMILES: COC(=O)Cc1ccccc1CN1CCCc2ccc(-c3cccc([C@@H](C)NC(=O)OC(C)(C)C)c3F)cc21]

The title compound was synthesized as described in Intermediate 130-D starting with methyl 2-(2-((7-bromo-3,4-dihydroquinolin-1(2H)-yl)methyl)phenyl)acetate and (R)-tert-butyl (1-(2-fluoro-3-(4,4,5,5-tetramethyl-1,3,2-dioxaborolan-2-yl)phenyl)ethyl)carbamate (Intermediate 27-B). MS (ESI+) m/z: 533.3 (M+H).

### Example 219-C

### (R)-Methyl 2-(2-((7-(3-(1-aminoethyl)-2-fluorophenyl)-3,4-dihydroquinolin-1(2H)-yl)methyl)phenyl)acetate

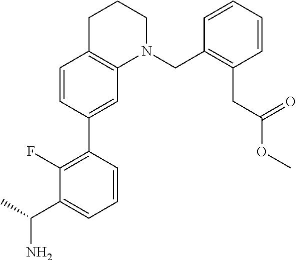 [SMILES: COC(=O)Cc1ccccc1CN1CCCc2ccc(-c3cccc([C@@H](C)N)c3F)cc21]

The title compound was synthesized as described in example 77B starting with (R)-methyl 2-(2-((7-(3-(1-((tert-butoxycarbonyl)amino)ethyl)-2-fluorophenyl)-3,4-dihydroquinolin-1(2H)-yl)methyl)phenyl)acetate. MS (ESI+) m/z: 433.2 (M+H).

### Example 219-D

### (R)-2-(2-((7-(3-(1-Aminoethyl)-2-fluorophenyl)-3,4-dihydroquinolin-1(2H)-yl)methyl)phenyl)acetic acid

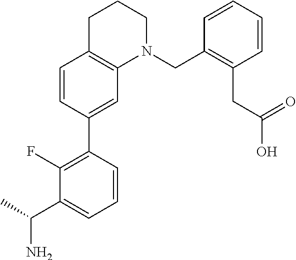 [SMILES: C[C@@H](N)c1cccc(-c2ccc3c(c2)N(Cc2ccccc2CC(=O)O)CCC3)c1F]

To a solution with (R)-methyl 2-(2-((7-(3-(1-aminoethyl)-2-fluorophenyl)-3,4-dihydroquinolin-1(2H)-yl)methyl)phenyl)acetate (45 mg, 0.104 mmol) in CH₃CN (2 ml) was added LiOH (1 M, 0.468 ml, 0.468 mmol). The reaction mixture was stirred for 16 hr at rt. The organic layer was filtered and concentrated before the residue was purified using preparative HPLC method B for purification. ¹H NMR (400 MHz, Methanol-d₄) δ ppm 7.32 (tdd, J=8.1, 8.1, 6.2, 1.8 Hz, 2H), 7.20-7.06 (m, 5H), 7.02-6.98 (m, 1H), 6.66-6.58 (m, 2H), 4.68-4.49 (m, 3H), 3.61-3.43 (m, 4H), 2.86 (q, J=5.3, 5.3, 4.4 Hz, 2H), 2.06 (tt, J=10.3, 10.3, 6.0, 6.0 Hz, 2H), 1.61 (d, J=6.9 Hz, 3H). HRMS calcd. for C₂₆H₂₇FN₂O₂(M+H)⁺ 419.2135. found 419.2132.

### Example 220

### Example 220-A

### Methyl 2-(2-(((3-bromophenyl)(methyl)amino)methyl)phenyl)acetate

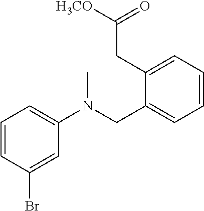 [SMILES: COC(=O)Cc1ccccc1CN(C)c1cccc(Br)c1]

Sodium triacetoxyborohydride (538 mg, 2.54 mmol) was added to a solution of 3-bromo-N-methylaniline (236 mg, 1.27 mmol) and methyl 2-(2-formylphenyl)acetate (226 mg, 1.27 mmol) in DCE (4 mL) at room temperature. The reaction mixture was stirred at room temperature overnight and LC-MS showed about 50% conversion. The reaction mixture was partitioned between EtOAc and sat. NH₄Cl. The aqueous layer was extracted with EtOAc. The combined organics were washed with brine, dried (Na₂SO₄) and concentrated. The residue was purified by silica gel chromatography (EtOAc-heptane 0-30%) to provide the desired product. MS (ESI+) m/z 348.1, 350.1 (M+1).

### Example 220-B

### (R)-methyl 2-(2-(((3′-(1-((tert-butoxycarbonyl)amino)ethyl)-2′-fluoro-[1,1′-biphenyl]-3-yl)(methyl)amino)methyl)phenyl)acetate

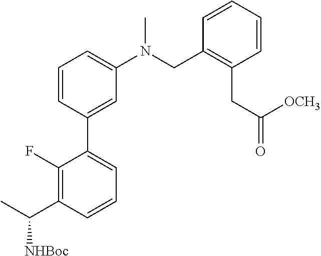 [SMILES: COC(=O)Cc1ccccc1CN(C)c1cccc(-c2cccc([C@@H](C)NC(=O)OC(C)(C)C)c2F)c1]

A mixture of methyl 2-(2-(((3-bromophenyl)(methyl)amino)methyl)phenyl)acetate (114 mg, 0.327 mmol), (R)-tert-butyl (1-(2-fluoro-3-(4,4,5,5-tetramethyl-1,3,2-dioxaborolan-2-yl)phenyl)ethyl)carbamate (Intermediate 27-B) (143 mg, 0.393 mmol), K₃PO₄ (0.491 mL, 0.982 mmol) and PdCl₂(dppf).CH₂Cl₂ adduct (13.37 mg, 0.016 mmol) in CH₃CN (3 mL) was heated in microwave at 110° C. for 1 hr. The resulting mixture was partitioned between EtOAc and sat. NH₄Cl. The aqueous layer was extracted with EtOAc. The combined organics were washed with brine, dried (Na₂SO₄) and concentrated. The residue was purified by silica gel chromatography (EtOAc-heptane 0-30%) to provide the desired product. MS (ESI+) m/z 507.5 (M+1).

### Example 220-C

### (R)-methyl 2-(2-(((3′-(1-aminoethyl)-2′-fluoro-[1,1′-biphenyl]-3-yl)(methyl)amino)methyl)phenyl)acetate

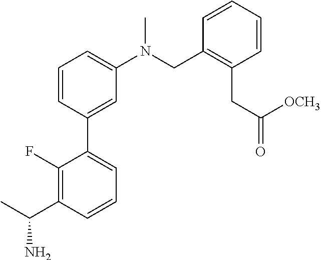 [SMILES: COC(=O)Cc1ccccc1CN(C)c1cccc(-c2cccc([C@@H](C)N)c2F)c1]

TFA (1 mL, 12.98 mmol) was added to a solution of (R)-methyl 2-(2-(((3′-(1-((tert-butoxycarbonyl)amino)ethyl)-2′-fluoro-[1,1′-biphenyl]-3-yl)(methyl)amino)methyl)phenyl)acetate (159 mg, 0.314 mmol) in DCM (3 mL) at room temperature. After 1 hr, the reaction mixture was concentrated. The residue was dissolved in CH₃CN and the solution was passed through a HCO₃⁻ SCX cartridge to remove any residual TFA. The filtrate was concentrated and the residue was used directly in the next reaction. MS (ESI+) m/z 407.3 (M+1).

### Example 220-D

### (R)-2-(2-(((3′-(1-aminoethyl)-2′-fluoro-[1,1′-biphenyl]-3-yl)(methyl)amino)methyl)phenyl)acetic acid

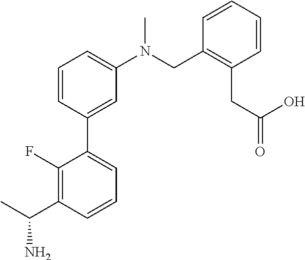 [SMILES: C[C@@H](N)c1cccc(-c2cccc(N(C)Cc3ccccc3CC(=O)O)c2)c1F]

LiOH (1 ml, 1.000 mmol) was added to a solution of (R)-methyl 2-(2-(((3′-(1-aminoethyl)-2′-fluoro-[1,1′-biphenyl]-3-yl)(methyl)amino)methyl)phenyl)acetate (128 mg, 0.314 mmol) in acetonitrile (2 mL) and the resulting mixture was stirred at room temperature overnight. The mixture was then filtered and the filtrate was loaded onto and purified by reverse HPLC (CH₃CN-water (0.1% NH₄OH) 10-60%) to provide the desired product. ¹H NMR (400 MHz, DMSO-d₆) δ ppm 7.36 (dtd, J=11.8, 7.3, 1.8 Hz, 2H), 7.28-6.99 (m, 6H), 6.90-6.67 (m, 3H), 4.66 (d, J=5.8 Hz, 2H), 4.54 (q, J=6.8 Hz, 1H), 3.56 (s, 2H), 3.09 (s, 3H), 1.54 (d, J=6.8 Hz, 3H). HRMS calcd. for C₂₄H₂₅FN₂O₂(M+H)⁺ 393.1978. found 393.1967.

### Example 221

### a) (R)-2-(2-((5-amino-3′-(1-aminoethyl)-2′,4-difluoro-[1,1′-biphenyl]-3-yl)methoxy)phenyl)acetic acid and b) 2-(2-((3′-((R)-1-aminoethyl)-2′,4-difluoro-5-(((tetrahydrofuran-2-yl)methyl)amino)-[1,1′-biphenyl]-3-yl)methoxy)phenyl)acetic acid (mixture of diastereomers)

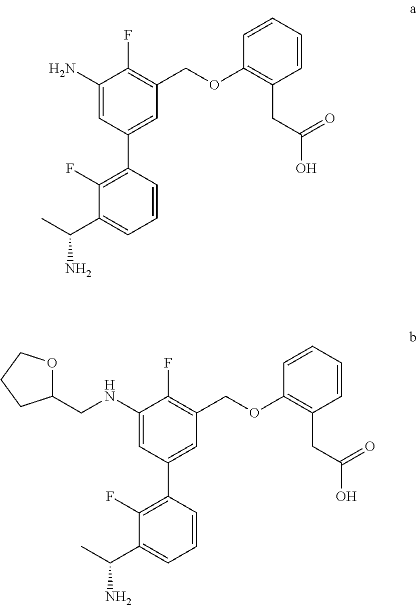 [SMILES: C[C@@H](N)c1cccc(-c2cc(N)c(F)c(COc3ccccc3CC(=O)O)c2)c1F ; C[C@@H](N)c1cccc(-c2cc(COc3ccccc3CC(=O)O)c(F)c(NCC3CCCO3)c2)c1F]

The title compounds were synthesized in the same manner as Examples 154-C using (R)-tert-butyl (1-(2-fluoro-3-(4,4,5,5-tetramethyl-1,3,2-dioxaborolan-2-yl)phenyl)ethyl)carbamate (Intermediate 27-B) instead of (3-(((tert-butoxycarbonyl)amino)methyl)phenyl)boronic acid.

a): ¹H NMR (400 MHz, METHANOL-d₄) δ ppm 7.45 (dtd, J=22.3, 7.2, 6.7, 1.8 Hz, 2H), 7.28 (t, J=7.7 Hz, 1H), 7.23-7.11 (m, 3H), 7.01-6.92 (m, 2H), 6.88 (td, J=7.4, 1.1 Hz, 1H), 5.20 (s, 2H), 4.74 (m, 1H), 3.57 (s, 2H), 1.67 (d, J=6.9 Hz, 3H). HRMS calcd. for C₂₃H₂₂F₂N₂O₃ (M+H)⁺ 413.1677. found 413.1680.

b): (mixture of diastereomers): ¹H NMR (400 MHz, METHANOL-d₄) δ ppm 7.51 (t, J=6.7 Hz, 1H), 7.38 (d, J=1.8 Hz, 1H), 7.29-7.15 (m, 3H), 7.11-7.03 (m, 1H), 6.99-6.81 (m, 3H), 5.46 (d, J=7.0 Hz, 1H), 5.16 (s, 2H), 4.43 (t, J=6.6 Hz, 1H), 4.15-3.99 (m, 1H), 3.77 (ddd, J=8.1, 7.0, 6.0 Hz, 1H), 3.64 (q, J=7.4 Hz, 1H), 3.51 (s, 2H), 3.19 (dt, J=12.7, 6.0 Hz, 1H), 1.84 (t, J=6.6 Hz, 3H), 1.69-1.56 (m, 1H), 1.39 (d, J=6.7 Hz, 3H). HRMS calcd. for C₂₈H₃₀F₂N₂O₄(M+H)⁺ 497.2252. found 497.2236.

### Example 222

### 2-(2-((3′-((R)-1-aminoethyl)-2′,6-difluoro-5-(((tetrahydrofuran-2-yl)methyl)amino)-[1,1′-biphenyl]-3-yl)methoxy)phenyl)acetic acid

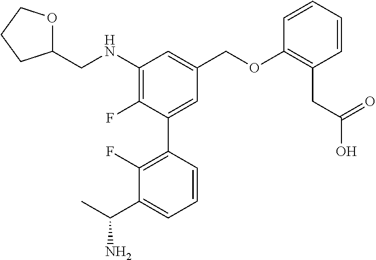 [SMILES: C[C@@H](N)c1cccc(-c2cc(COc3ccccc3CC(=O)O)cc(NCC3CCCO3)c2F)c1F]

The title compounds were synthesized in the same manner as Examples 154-B using (R)-tert-butyl (1-(2-fluoro-3-(4,4,5,5-tetramethyl-1,3,2-dioxaborolan-2-yl)phenyl)ethyl)carbamate (Intermediate 27-B) instead of (3-(((tert-butoxycarbonyl)amino)methyl)phenyl)boronic acid. ¹H NMR (400 MHz, METHANOL-d₄) δ ppm 7.58-7.42 (m, 2H), 7.31 (t, J=7.7 Hz, 1H), 7.18 (dtd, J=18.1, 7.5, 2.5 Hz, 2H), 7.02-6.78 (m, 4H), 5.06 (s, 2H), 4.74 (t, J=6.9 Hz, 1H), 4.15 (dt, J=7.0, 3.5 Hz, 1H), 3.89 (dt, J=8.2, 6.6 Hz, 1H), 3.77 (td, J=7.8, 6.4 Hz, 1H), 3.57 (s, 2H), 3.37 (dd, J=13.2, 4.1 Hz, 1H), 3.21 (dd, J=13.2, 7.1 Hz, 1H), 2.16-1.83 (m, 3H), 1.78-1.61 (m, 4H). HRMS calcd. for C₂₈H₃₀F₂N₂O₄(M+H)⁺ 497.2240. found 497.2237.

### Example 223

### Example 223-A

### (R)-tert-Butyl 2-(2-((3′-(1-((tert-butoxycarbonyl)amino)ethyl)-2′-fluoro-5-((2-methoxyethyl)amino)-[1,1′-biphenyl]-3-yl)methoxy)phenyl)acetate

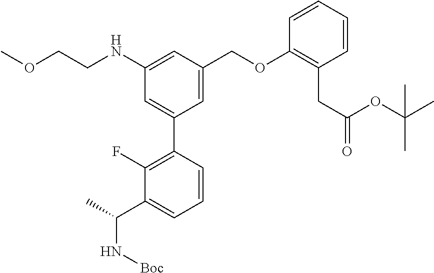 [SMILES: COCCNc1cc(COc2ccccc2CC(=O)OC(C)(C)C)cc(-c2cccc([C@@H](C)NC(=O)OC(C)(C)C)c2F)c1]

The title compound was synthesized in the same manner as Example 150-B starting with (R)-tert-Butyl 2-(2-((3′-(1-((tert-butoxycarbonyl)amino)ethyl)-5-chloro-2′-fluoro-[1,1′-biphenyl]-3-yl)methoxy)phenyl)acetate (Example 150-A) and 2-methoxyethanamine. MS (ESI+) m/z 609.4 (M+H).

### Example 223-B

### (R)-2-(2-((3′-(1-aminoethyl)-2′-fluoro-5-((2-methoxyethyl)amino)-[1,1′-biphenyl]-3-yl)methoxy)phenyl)acetic acid

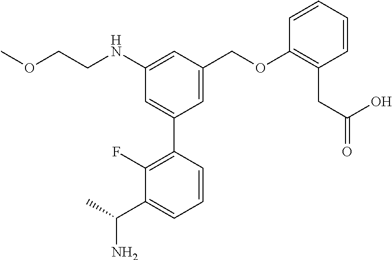 [SMILES: COCCNc1cc(COc2ccccc2CC(=O)O)cc(-c2cccc([C@@H](C)N)c2F)c1]

The title compound was synthesized in the same manner as Example 114-B. ¹H NMR (400 MHz, DMSO-d₆) δ ppm 7.52 (td, J=7.21, 1.59 Hz, 1H) 7.31 (td, J=7.49, 1.77 Hz, 1H) 7.14-7.25 (m, 3H) 6.97-7.06 (m, 1H) 6.87 (td, J=7.40, 0.98 Hz, 1H) 6.79 (s, 1H) 6.61-6.71 (m, 2H) 5.66-5.79 (m, 1H) 5.05 (s, 2H) 4.27-4.41 (m, 1H) 3.54 (s, 2H) 3.50 (t, J=5.62 Hz, 2H) 3.28 (s, 3H) 3.20-3.26 (m, 2H) 1.32 (d, J=6.60 Hz, 3H). HRMS calcd. for C₂₆H₂₉FN₂O₄ (M+H)⁺ 453.2190. found 453.2185.

### Example 224

### Example 224-A

### (R)-tert-Butyl 2-(2-((3′-(1-((tert-butoxycarbonyl)amino)ethyl)-2′-fluoro-5-(isobutylamino)-[1,1′-biphenyl]-3-yl)methoxy)phenyl)acetate

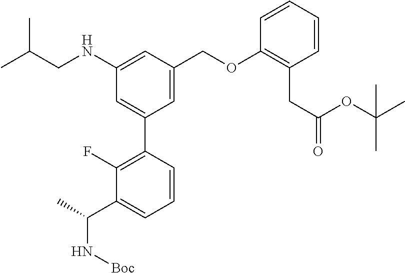 [SMILES: CC(C)CNc1cc(COc2ccccc2CC(=O)OC(C)(C)C)cc(-c2cccc([C@@H](C)NC(=O)OC(C)(C)C)c2F)c1]

The title compound was synthesized in the same manner as Example 150-B starting with (Example 150-A) (R)-tert-Butyl 2-(2-((3′-(1-((tert-butoxycarbonyl)amino)ethyl)-5-chloro-2′-fluoro-[1,1′-biphenyl]-3-yl)methoxy)phenyl)acetate and 2-methylpropan-1-amine. MS (ESI+) m/z 551.5 (M−t-Butyl)⁺.

### Example 224-B

### (R)-2-(2-((3′-(1-aminoethyl)-2′-fluoro-5-(isobutylamino)-[1,1′-biphenyl]-3-yl)methoxy)phenyl)acetic acid

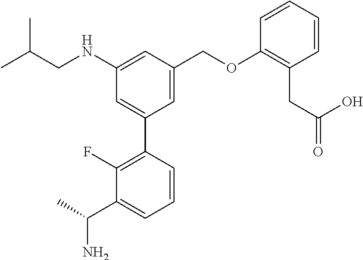 [SMILES: CC(C)CNc1cc(COc2ccccc2CC(=O)O)cc(-c2cccc([C@@H](C)N)c2F)c1]

The title compound was synthesized in the same manner as Example 114-B. ¹H NMR (400 MHz, DMSO-d₆) δ ppm 7.52 (td, J=7.21, 1.59 Hz, 1H) 7.29 (td, J=7.49, 1.77 Hz, 1H) 7.14-7.24 (m, 3H) 7.02 (d, J=7.95 Hz, 1H) 6.83-6.91 (m, 1H) 6.76 (s, 1H) 6.65 (s, 2H) 5.77 (t, J=5.56 Hz, 1H) 5.04 (s, 2H) 4.33 (q, J=6.56 Hz, 1H) 3.55 (s, 2H) 2.82-2.90 (m, 2H) 1.85 (dt, J=13.36, 6.71 Hz, 1H) 1.32 (d, J=6.60 Hz, 3H) 0.94 (d, J=6.60 Hz, 6H) HRMS calcd. for C₂₇H₃₁FN₂O₃(M+H)⁺ 451.2397. found 451.2389.

### Example 225

### Example 225-A

### (R)-tert-Butyl 2-(2-((3-(6-(1-((tert-butoxycarbonyl)amino)ethyl)pyridin-2-yl)-5-chlorobenzyl)oxy)phenyl)acetate

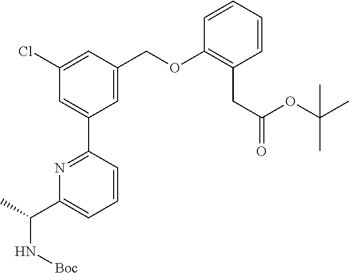 [SMILES: C[C@@H](NC(=O)OC(C)(C)C)c1cccc(-c2cc(Cl)cc(COc3ccccc3CC(=O)OC(C)(C)C)c2)n1]

The title compound was synthesized in the same manner as Example 141-A starting with tert-butyl 2-(2-((3-bromo-5-chlorobenzyl)oxy)phenyl)acetate (Intermediate 91) and (R)-tert-butyl (1-(6-bromopyridin-2-yl)ethyl)carbamate (Intermediate 147). MS (ESI+) m/z 553.4, 555.4 (M+H).

### Example 225-B

### tert-Butyl 2-(2-((3-(6-((R)-1-((tert-butoxycarbonyl)amino)ethyl)pyridin-2-yl)-5-((((R)-tetrahydrofuran-2-yl)methyl)amino)benzyl)oxy)phenyl)acetate

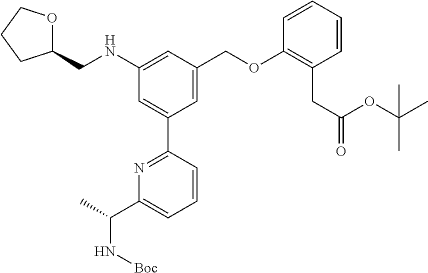 [SMILES: C[C@@H](NC(=O)OC(C)(C)C)c1cccc(-c2cc(COc3ccccc3CC(=O)OC(C)(C)C)cc(NC[C@H]3CCCO3)c2)n1]

The title compound was synthesized in the same manner as Example 150-B. MS (ESI+) m/z 618.6 (M+H).

### Example 225-C

### 2-(2-((3-(6-((R)-1-aminoethyl)pyridin-2-yl)-5-((((R)-tetrahydrofuran-2-yl)methyl)amino)benzyl)oxy)phenyl)acetic acid

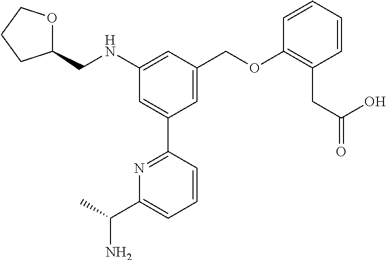 [SMILES: C[C@@H](N)c1cccc(-c2cc(COc3ccccc3CC(=O)O)cc(NC[C@H]3CCCO3)c2)n1]

The title compound was synthesized in the same manner as Example 114-B. ¹H NMR (400 MHz, DMSO-d₆) δ ppm 7.79-7.87 (m, 2H) 7.72 (d, J=7.70 Hz, 1H) 7.32 (d, J=7.58 Hz, 1H) 7.16 (s, 1H) 7.04-7.13 (m, 2H) 6.88 (d, J=7.82 Hz, 1H) 6.80 (td, J=7.34, 0.73 Hz, 1H) 6.69 (s, 1H) 5.65 (t, J=5.26 Hz, 1H) 5.06 (s, 2H) 4.29 (q, J=6.81 Hz, 1H) 3.98-4.08 (m, 1H) 3.77-3.86 (m, 1H) 3.60-3.71 (m, 1H) 3.29-3.43 (m, 2H) 3.08-3.26 (m, 2H) 1.94-2.05 (m, 1H) 1.76-1.93 (m, 2H) 1.62 (ddt, J=11.92, 8.68, 6.88, 6.88 Hz, 1H) 1.46 (d, J=6.85 Hz, 3H). HRMS calcd. for C₂₇H₃₁N₃O₄ (M−H)⁺ 460.2236. found 460.2232.

### Example 226

### Example 226-A

### tert-Butyl 2-(2-((3-chloro-5-((2,2,2-trifluoroethyl)amino)benzyl)oxy)phenyl)acetate

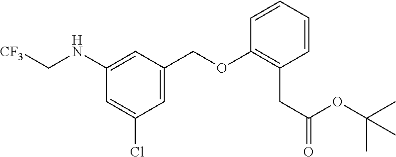 [SMILES: CC(C)(C)OC(=O)Cc1ccccc1OCc1cc(Cl)cc(NCC(F)(F)F)c1]

To tert-Butyl 2-(2-((3-bromo-5-chlorobenzyl)oxy)phenyl)acetate (Intermediate 53) (0.27 g, 0.656 mmol), 2,2,2-trifluoroethanamine (0.103 ml, 1.312 mmol), BrettPhos+Pd (11) (0.026 g, 0.033 mmol) and Cs₂CO₃ (0.641 g, 1.967 mmol) in CH3CN (2 mL) was heated at 110° C. for 30 min. Added another 0.050 mL of 2,2,2-trifluoroethanamine and continued heating. The reaction was cooled to room temperature and diluted with EA and water, Removed EA, dried, concentrated and purified via fcc (0-50% EA:heptanes) to obtain title compound. MS (ESI+) m/z 374.1, 376.1. (M−t-butyl)⁺.

### Example 226-B

### (R)-tert-Butyl 2-(2-((3′-(1-((tert-butoxycarbonyl)amino)ethyl)-2′-fluoro-5-((2,2,2-trifluoroethyl)amino)-[1,1′-biphenyl]-3-yl)methoxy)phenyl)acetate

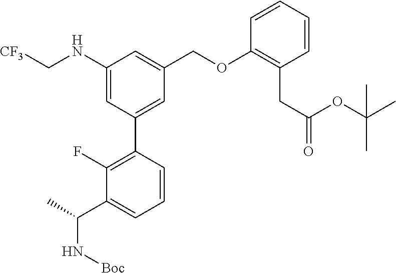 [SMILES: C[C@@H](NC(=O)OC(C)(C)C)c1cccc(-c2cc(COc3ccccc3CC(=O)OC(C)(C)C)cc(NCC(F)(F)F)c2)c1F]

The title compound was synthesized in the same manner as Example 141-A from tert-butyl 2-(2-((3-chloro-5-((2,2,2-trifluoroethyl)amino)benzyl)oxy)phenyl)acetate and Intermediate 27-B. MS (ESI+) m/z 633.3 (M+H).

### Example 226-C

### (R)-2-(2-((3′-(1-aminoethyl)-2′-fluoro-5-((2,2,2-trifluoroethyl)amino)-[1,1′-biphenyl]-3-yl)methoxy)phenyl)acetic acid

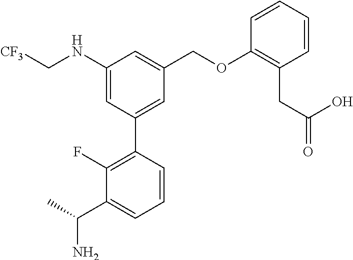 [SMILES: C[C@@H](N)c1cccc(-c2cc(COc3ccccc3CC(=O)O)cc(NCC(F)(F)F)c2)c1F]

The title compound was synthesized in the same manner as Example 114-B from (R)-tert-butyl 2-(2-((3′-(1-((tert-butoxycarbonyl)amino)ethyl)-2′-fluoro-5-((2,2,2-trifluoroethyl)amino)-[1,1′-biphenyl]-3-yl)methoxy)phenyl)acetate. ¹H NMR (400 MHz, DMSO-d₆) δ ppm 7.53 (td, J=7.24, 1.65 Hz, 1H) 7.31 (td, J=7.46, 1.71 Hz, 1H) 7.10-7.25 (m, 3H) 6.96-7.05 (m, 1H) 6.75-6.93 (m, 4H) 6.37 (t, J=6.97 Hz, 1H) 5.06 (s, 2H) 4.33 (d, J=6.72 Hz, 1H) 3.96 (dd, J=9.54, 6.97 Hz, 2H) 3.53 (br. s., 2H) 1.31 (d, J=6.60 Hz, 3H). HRMS calcd. for C₂₅H₂₄F₄N₂O₃(M−H)⁺475.1645. found 475.1651.

### Example 227

### (R)-2-(2-((3′-(1-aminoethyl)-2′-fluoro-5-(((1-methylcyclopropyl)methyl)amino)-[1,1′-biphenyl]-3-yl)methoxy)phenyl)acetic acid

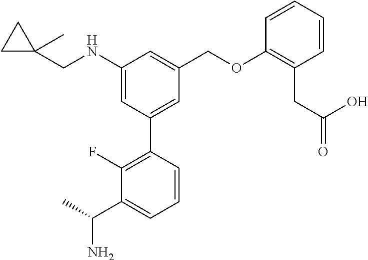 [SMILES: C[C@@H](N)c1cccc(-c2cc(COc3ccccc3CC(=O)O)cc(NCC3(C)CC3)c2)c1F]

The title compound was synthesized in the same manner as Example 226 from tert-Butyl 2-(2-((3-bromo-5-chlorobenzyl)oxy)phenyl)acetate (Intermediate 53) and (1-methylcyclopropyl)methanamine hydrochloride (CAS #98137-40-7). ¹H NMR (400 MHz, DMSO-d₆) δ ppm 7.47-7.55 (m, 1H) 7.29 (dd, J=7.52, 1.77 Hz, 1H) 7.15-7.24 (m, 3H) 7.02 (d, J=7.83 Hz, 1H) 6.88 (td, J=7.40, 0.86 Hz, 1H) 6.76 (s, 1H) 6.65-6.70 (m, 2H) 5.69 (s, 1H) 5.04 (s, 2H) 4.34 (d, J=6.72 Hz, 1H) 3.55 (br. s., 2H) 2.95 (d, J=5.26 Hz, 2H) 1.32 (d, J=6.60 Hz, 3H) 1.12 (s, 3H) 0.40-0.46 (m, 2H) 0.24-0.31 (m, 2H). HRMS calcd. for C₂₈H₃₁FN₂O₃ (M+H)⁺ 463.2397. found 463.2398.

### Example 228

### Example 228-A

### tert-Butyl 2-(2-((3-acetamido-5-chlorobenzyl)oxy)phenyl)acetate

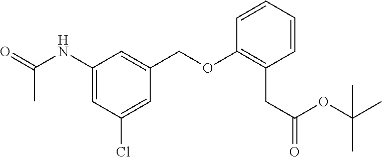 [SMILES: CC(=O)Nc1cc(Cl)cc(COc2ccccc2CC(=O)OC(C)(C)C)c1]

To tert-Butyl 2-(2-((3-bromo-5-chlorobenzyl)oxy)phenyl)acetate (Intermediate 53) (0.16 g, 0.389 mmol) in t-butanol (2.398 mL) was added K₃PO₄ (0.583 mL, 1.166 mmol), Pd(OAc)₂ (8.72 mg, 0.039 mmol), acetamide (0.046 g, 0.777 mmol) and t-butylBrettPhos (0.019 g, 0.039 mmol) and was degassed and purged with nitrogen and heated at 110° C. for 30 minutes. The reaction was diluted with EA and water and the EA layer was removed, dried and concentrated and absorbed onto silica to purify via FCC (0-100% EA:heptanes) to obtain the title compound. MS (ESI+) m/z 388.2, 390.2 (M−H)⁺.

### Example 228-B

### (R)-2-(2-((5-acetamido-3′-(1-aminoethyl)-2′-fluoro-[1,1′-biphenyl]-3-yl)methoxy)phenyl)acetic acid

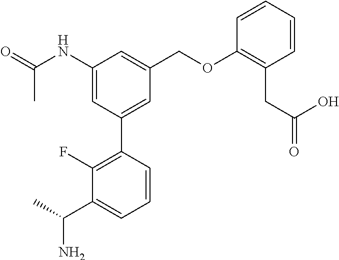 [SMILES: CC(=O)Nc1cc(COc2ccccc2CC(=O)O)cc(-c2cccc([C@@H](C)N)c2F)c1]

The title compound was synthesized in the same manner as Example 226. ¹H NMR (400 MHz, DMSO-d₆) δ ppm 10.07 (s, 1H) 7.73 (s, 1H) 7.65 (s, 1H) 7.51-7.59 (m, 1H) 7.29-7.36 (m, 2H) 7.23-7.28 (m, 1H) 7.16-7.22 (m, 2H) 6.99-7.06 (m, 1H) 6.88 (td, J=7.37, 0.92 Hz, 1H) 5.15 (s, 2H) 4.35 (d, J=6.72 Hz, 1H) 3.54 (s, 2H) 2.06 (s, 3H) 1.34 (s, 3H). HRMS calcd. for C₂₅H₂₅FN₂O₄(M−H) 435.1720. found 435.1737.

### Example 229

### (R)-2-(2-((3′-(1-aminoethyl)-2′-fluoro-5-(N-methylacetamido)-[1,1′-biphenyl]-3-yl)methoxy)phenyl)acetic acid

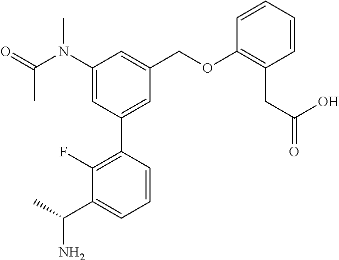 [SMILES: CC(=O)N(C)c1cc(COc2ccccc2CC(=O)O)cc(-c2cccc([C@@H](C)N)c2F)c1]

The title compound was synthesized in the same manner as Example 226. ¹H NMR (400 MHz, CHLOROFORM-d) δ ppm 7.93 (br. s., 1H) 7.34 (td, J=7.40, 1.71 Hz, 1H) 7.13-7.26 (m, 6H) 6.85-6.98 (m, 2H) 5.21 (s, 2H) 4.39 (q, J=6.72 Hz, 1H) 3.67 (s, 2H) 3.33 (s, 3H) 1.96 (s, 3H) 1.57 (d, J=6.72 Hz, 3H). HRMS calcd. for C₂₆H₂₇FN₂O₄(M−H)⁺ 449.1877. found 449.1896.

### Example 230

### (R)-2-(2-((3′-(1-aminoethyl)-2′-fluoro-5-(1-methyl-1H-pyrazol-4-yl)-[1,1′-biphenyl]-3-yl)methoxy)phenyl)acetic acid

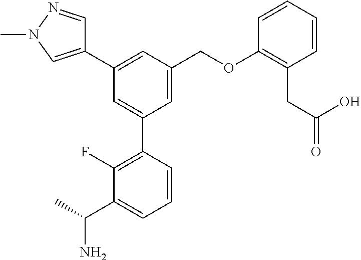 [SMILES: C[C@@H](N)c1cccc(-c2cc(COc3ccccc3CC(=O)O)cc(-c3cnn(C)c3)c2)c1F]

The title compound was synthesized in the same manner as Example 103 starting from (R)-tert-Butyl 2-(2-((3′-(1-((tert-butoxycarbonyl)amino)ethyl)-5-chloro-2′-fluoro-[1,1′-biphenyl]-3-yl)methoxy)phenyl)acetate (Example 150-A). ¹H NMR (400 MHz, CHLOROFORM-d) δ ppm 7.80 (d, J=0.61 Hz, 1H) 7.76 (br. s., 1H) 7.66 (s, 1H) 7.47 (s, 1H) 7.35-7.42 (m, 2H) 7.10-7.26 (m, 4H) 6.84-6.91 (m, 2H) 5.17 (s, 2H) 4.36 (d, J=6.60 Hz, 1H) 3.95 (s, 3H) 3.62 (s, 2H) 1.50 (d, J=6.72 Hz, 3H). HRMS calcd. for C₂₇H₂₆FN₃O₃(M+H)⁺ 460.2036. found 460.2024.

### Example 231

### Example 231-A

### (R)-tert-Butyl 2-(2-((3′-(1-((tert-butoxycarbonyl)amino)ethyl)-2′-fluoro-5-(2,2,2-trifluoroethoxy)-[1,1′-biphenyl]-3-yl)methoxy)phenyl)acetate

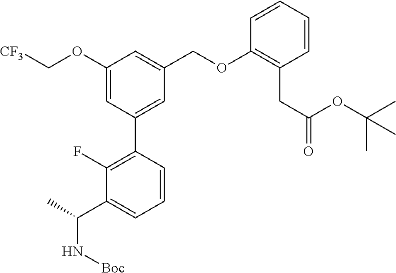 [SMILES: C[C@@H](NC(=O)OC(C)(C)C)c1cccc(-c2cc(COc3ccccc3CC(=O)OC(C)(C)C)cc(OCC(F)(F)F)c2)c1F]

The title compound was synthesized in the same manner as Example 141-A from tert-Butyl 2-(2-((3-bromo-5-(2,2,2-trifluoroethoxy)benzyl)oxy)phenyl)acetate (Intermediate 58-C) and Intermediate 27-B. MS (ESI+) m/z 632.3 (M−H).

### Example 231-B

### (R)-2-(2-((3′-(1-aminoethyl)-2′-fluoro-5-(2,2,2-trifluoroethoxy)-[1,1′-biphenyl]-3-yl)methoxy)phenyl)acetic acid

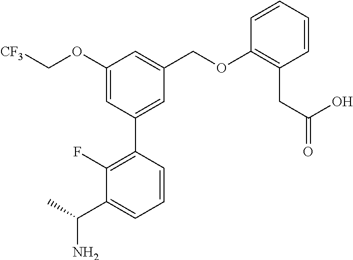 [SMILES: C[C@@H](N)c1cccc(-c2cc(COc3ccccc3CC(=O)O)cc(OCC(F)(F)F)c2)c1F]

The title compound was synthesized in the same manner as Example 114-B from (R)-tert-butyl 2-(2-((3′-(1-((tert-butoxycarbonyl)amino)ethyl)-2′-fluoro-5-(2,2,2-trifluoroethoxy)-[1,1′-biphenyl]-3-yl)methoxy)phenyl)acetate. ¹H NMR (400 MHz, DMSO-d₆) δ ppm 7.52-7.61 (m, 1H) 7.37-7.44 (m, 1H) 7.35 (s, 1H) 7.16-7.30 (m, 4H) 7.14 (s, 1H) 7.01 (d, J=7.95 Hz, 1H) 6.89 (td, J=7.40, 0.86 Hz, 1H) 5.18 (s, 2H) 4.83 (q, J=8.93 Hz, 2H) 4.35 (d, J=6.60 Hz, 1H) 3.55 (s, 2H) 1.34 (d, J=6.72 Hz, 3H). HRMS calcd. for C₂₅H₂₃F₄NO₄ (M+H)⁺ 478.1641. found 478.1631.

### Example 231-E

### tert-butyl 2-(2-((3′-((S)-1-amino-2-hydroxyethyl)-2′-fluoro-5-(((R)-tetrahydrofuran-2-yl)methoxy)-[1,1′-biphenyl]-3-yl)methoxy)phenyl)acetate

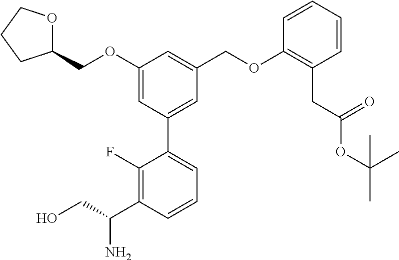 [SMILES: CC(C)(C)OC(=O)Cc1ccccc1OCc1cc(OC[C@H]2CCCO2)cc(-c2cccc([C@H](N)CO)c2F)c1]

The title compound was synthesized in the same manner as Example 141-A from (R)-tert-butyl 2-(2-((3-((tetrahydrofuran-2-yl)methoxy)-5-(4,4,5,5-tetramethyl-1,3,2-dioxaborolan-2-yl)benzyl)oxy)phenyl)acetate (Intermediate 150) and (S)-2-amino-2-(3-chloro-2-fluorophenyl)ethanol hydrochloride (CAS #1391506-22-1). MS (ESI+) m/z 552.5 (M+H).

### Example 231-F

### 2-(2-((3′-((S)-1-amino-2-hydroxyethyl)-2′-fluoro-5-(((R)-tetrahydrofuran-2-yl)methoxy)-[1,1′-biphenyl]-3-yl)methoxy)phenyl)acetic acid

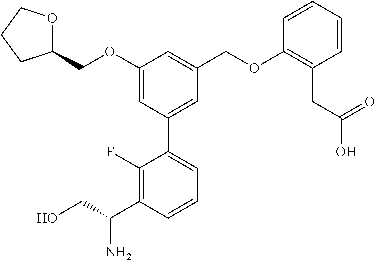 [SMILES: N[C@H](CO)c1cccc(-c2cc(COc3ccccc3CC(=O)O)cc(OC[C@H]3CCCO3)c2)c1F]

The title compound was synthesized in the same manner as Example 114-B from tert-butyl 2-(2-((3′-((S)-1-amino-2-hydroxyethyl)-2′-fluoro-5-(((R)-tetrahydrofuran-2-yl)methoxy)-[1,1′-biphenyl]-3-yl)methoxy)phenyl)acetate. ¹H NMR (400 MHz, DMSO-d₆) δ ppm 7.55 (t, J=6.42 Hz, 1H) 7.38 (td, J=7.49, 1.53 Hz, 1H) 7.15-7.29 (m, 4H) 6.96-7.09 (m, 3H) 6.84-6.93 (m, 1H) 5.15 (s, 2H) 4.12-4.28 (m, 2H) 3.94-4.06 (m, 2H) 3.75-3.83 (m, 1H) 3.64-3.72 (m, 1H) 3.53-3.60 (m, 2H) 3.46 (d, J=7.46 Hz, 2H) 3.37 (dd, J=10.45, 7.52 Hz, 1H) 1.94-2.08 (m, 1H) 1.77-1.94 (m, 2H) 1.62-1.74 (m, 1H). HRMS calcd. for C₂₈H₃₀FNO₆ (M+H)⁺ 496.2135. found 496.2124.

### Example 232

### 2-(2-((3′-((R)-1-aminoethyl)-2′-fluoro-5-(((R)-tetrahydrofuran-2-yl)methoxy)-[1,1′-biphenyl]-3-yl)methoxy)phenyl)acetic acid

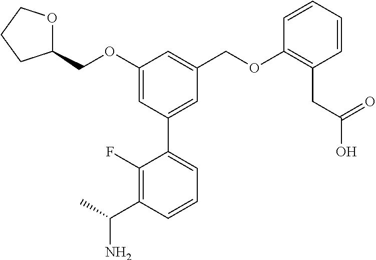 [SMILES: C[C@@H](N)c1cccc(-c2cc(COc3ccccc3CC(=O)O)cc(OC[C@H]3CCCO3)c2)c1F]

The title compound was synthesized in the same manner as Example 114 from (R)-tert-butyl 2-(2-((3-((tetrahydrofuran-2-yl)methoxy)-5-(4,4,5,5-tetramethyl-1,3,2-dioxaborolan-2-yl)benzyl)oxy)phenyl)acetate (Intermediate 150) and (R)-1-(3-chloro-2-fluorophenyl)ethanamine hydrochloride (CAS #1253792-97-0), S-Phos palladacycle was used instead of PdCl₂(dppf).CH₂Cl₂ adduct in the coupling step. ¹H NMR (400 MHz, DMSO-d₆) δ ppm 7.51-7.60 (m, 1H) 7.38 (td, J=7.55, 1.65 Hz, 1H) 7.15-7.29 (m, 4H) 7.05 (s, 1H) 6.96-7.03 (m, 2H) 6.83-6.92 (m, 1H) 5.15 (s, 2H) 4.35 (q, J=6.60 Hz, 1H) 4.12-4.22 (m, 1H) 3.92-4.07 (m, 2H) 3.75-3.83 (m, 1H) 3.64-3.73 (m, 1H) 3.55 (s, 2H) 1.95-2.06 (m, 1H) 1.77-1.94 (m, 2H) 1.62-1.75 (m, 1H) 1.33 (d, J=6.60 Hz, 3H). HRMS calcd. for C₂₈H₃₀FNO₅ (M+H)⁺ 480.2186. found 480.2166.

### Example 233

### 2-(2-((3′-((S)-1-aminoethyl)-2′-fluoro-5-(((R)-tetrahydrofuran-2-yl)methoxy)-[1,1′-biphenyl]-3-yl)methoxy)phenyl)acetic acid

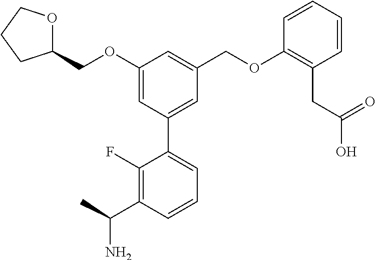 [SMILES: C[C@H](N)c1cccc(-c2cc(COc3ccccc3CC(=O)O)cc(OC[C@H]3CCCO3)c2)c1F]

The title compound was synthesized in the same manner as Example 232 using (S)-1-(3-chloro-2-fluorophenyl)ethanamine hydrochloride (CAS #1313593-59-7). ¹H NMR (400 MHz, DMSO-d₆) δ ppm 7.49-7.61 (m, 1H) 7.38 (td, J=7.55, 1.65 Hz, 1H) 7.14-7.29 (m, 4H) 7.06 (s, 1H) 7.01 (s, 2H) 6.88 (td, J=7.40, 0.86 Hz, 1H) 5.15 (s, 2H) 4.35 (q, J=6.60 Hz, 1H) 4.11-4.22 (m, 1H) 3.93-4.08 (m, 2H) 3.74-3.86 (m, 1H) 3.68 (td, J=7.70, 6.24 Hz, 1H) 3.54 (s, 2H) 1.93-2.06 (m, 1H) 1.76-1.93 (m, 2H) 1.60-1.75 (m, 1H) 1.33 (d, J=6.60 Hz, 3H). HRMS calcd. for C₂₈H₃₀FNO₅ (M+H)⁺ 480.2186. found 480.2163.

### Example 234

### Example 234-A

### (R)-tert-butyl 2-(2-((3′-(((tert-butoxycarbonyl)amino)methyl)-2′-fluoro-5-((tetrahydrofuran-2-yl)methoxy)-[1,1′-biphenyl]-3-yl)methoxy)phenyl)acetate

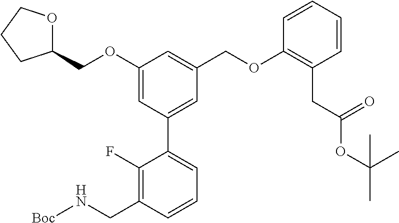 [SMILES: CC(C)(C)OC(=O)Cc1ccccc1OCc1cc(OC[C@H]2CCCO2)cc(-c2cccc(CNC(=O)OC(C)(C)C)c2F)c1]

The title compound was synthesized in the same manner as Example 141-A from (R)-tert-butyl 2-(2-((3-((tetrahydrofuran-2-yl)methoxy)-5-(4,4,5,5-tetramethyl-1,3,2-dioxaborolan-2-yl)benzyl)oxy)phenyl)acetate (Intermediate 150) and tert-butyl 3-chloro-2-fluorobenzylcarbamate (Intermediate 148). S-Phos palladacycle was used instead of PdCl₂(dppf).CH₂Cl₂ adduct in the coupling step. MS (ESI+) m/z 622.5 (M+H).

### Example 234-B

### (R)-2-(2-((3′-(aminomethyl)-2′-fluoro-5-((tetrahydrofuran-2-yl)methoxy)-[1,1′-biphenyl]-3-yl)methoxy)phenyl)acetic acid

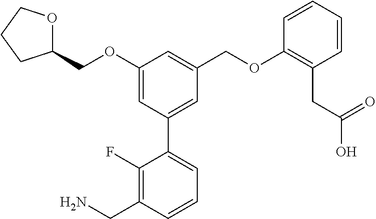 [SMILES: NCc1cccc(-c2cc(COc3ccccc3CC(=O)O)cc(OC[C@H]3CCCO3)c2)c1F]

The title compound was synthesized in the same manner as Example 114-B from (R)-tert-butyl 2-(2-((3′-(((tert-butoxycarbonyl)amino)methyl)-2′-fluoro-5-((tetrahydrofuran-2-yl)methoxy)-[1,1′-biphenyl]-3-yl)methoxy)phenyl)acetate. ¹H NMR (400 MHz, DMSO-d₆) δ ppm 7.35-7.52 (m, 2H) 7.14-7.28 (m, 4H) 7.05 (s, 1H) 6.96-7.02 (m, 2H) 6.87 (td, J=7.40, 0.86 Hz, 1H) 5.16 (s, 2H) 4.17 (qd, J=6.70, 3.97 Hz, 1H) 3.93-4.06 (m, 2H) 3.83 (s, 2H) 3.76-3.81 (m, 1H) 3.64-3.72 (m, 1H) 3.54 (s, 2H) 1.94-2.06 (m, 1H) 1.77-1.94 (m, 2H) 1.62-1.75 (m, 1H). HRMS calcd. for C₂₇H₂₈FNO₅ (M+H)⁺ 466.2030. found 466.2016.

### Example 235

### 2-(2-((3-(6-((R)-1-aminoethyl)pyridin-2-yl)-5-(((R)-tetrahydrofuran-2-yl)methoxy)benzyl)oxy)phenyl)acetic acid

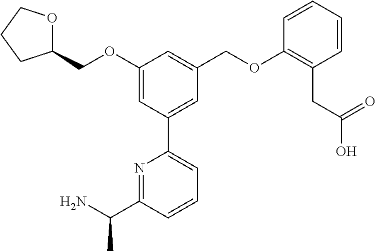 [SMILES: C[C@@H](N)c1cccc(-c2cc(COc3ccccc3CC(=O)O)cc(OC[C@H]3CCCO3)c2)n1]

The title compound was synthesized in the same manner as Example 234 using the appropriate intermediates. ¹H NMR (400 MHz, DMSO-d₆) δ ppm 8.28 (s, 1H) 7.79-7.94 (m, 2H) 7.47 (s, 1H) 7.36 (d, J=7.20 Hz, 1H) 7.02-7.15 (m, 3H) 6.75-6.91 (m, 2H) 5.15 (s, 2H) 4.32 (q, J=6.82 Hz, 1H) 4.15-4.25 (m, 1H) 3.98-4.12 (m, 2H) 3.77-3.87 (m, 1H) 3.71 (td, J=7.64, 6.19 Hz, 1H) 3.27-3.43 (m, 2H) 1.98-2.10 (m, 1H) 1.80-1.98 (m, 2H) 1.66-1.78 (m, 1H) 1.41-1.53 (m, 3H). HRMS calcd. for C₂₇H₃₀N₂O₅ (M−H)⁺ 461.2076. found 461.2091.

### Example 236

### Example 236-A

### (R)-tert-Butyl 2-(2-((3′-(1-((tert-butoxycarbonyl)amino)ethyl)-2′-fluoro-5-(2-(furan-2-yl)vinyl)-[1,1′-biphenyl]-3-yl)methoxy)phenyl)acetate

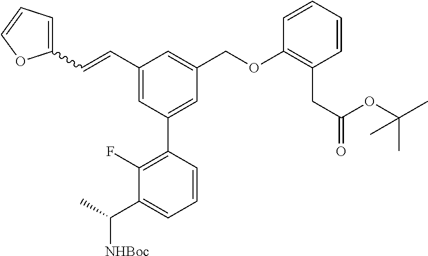 [SMILES: C[C@@H](NC(=O)OC(C)(C)C)c1cccc(-c2cc(C=Cc3ccco3)cc(COc3ccccc3CC(=O)OC(C)(C)C)c2)c1F]

The title compound was synthesized in the same manner as Example 141-A from tert-butyl 2-(2-((3-bromo-5-(2-(furan-2-yl)vinyl)benzyl)oxy)phenyl)acetate (Intermediate 153) and (R)-tert-butyl (1-(2-fluoro-3-(4,4,5,5-tetramethyl-1,3,2-dioxaborolan-2-yl)phenyl)ethyl)carbamate (Intermediate 27-B). MS (ESI+) m/z 472.3 (M−boc-t-butyl)⁺.

### Example 236-B

### tert-butyl 2-(2-((3′-((R)-1-((tert-butoxycarbonyl)amino)ethyl)-2′-fluoro-5-(2-(tetrahydrofuran-2-yl)ethyl)-[1,1′-biphenyl]-3-yl)methoxy)phenyl)acetate

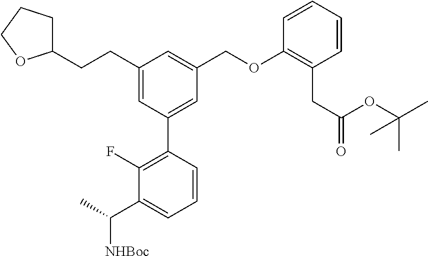 [SMILES: C[C@@H](NC(=O)OC(C)(C)C)c1cccc(-c2cc(CCC3CCCO3)cc(COc3ccccc3CC(=O)OC(C)(C)C)c2)c1F]

To (R)-tert-butyl 2-(2-((3′-(1-((tert-butoxycarbonyl)amino)ethyl)-2′-fluoro-5-(2-(furan-2-yl)vinyl)-[1,1′-biphenyl]-3-yl)methoxy)phenyl)acetate (0.3 g, 0.478 mmol) in MeOH (5 mL) was added 10% Pd-C (0.153 g, 0.143 mmol) and the mixture was degassed and placed under hydrogen (balloon) for 2 hours. The reaction was filtered and then concentrated, absorbed onto silica and purified via fcc (0-50% EA:heptanes) to obtain the title compound. MS (ESI+) m/z 578.5 (M−t-butyl)⁺.

### Example 236-C

### 2-(2-((3′-((R)-1-aminoethyl)-2′-fluoro-5-(2-(tetrahydrofuran-2-yl)ethyl)-[1,1′-biphenyl]-3-yl)methoxy)phenyl)acetic acid

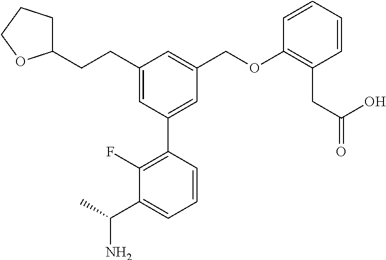 [SMILES: C[C@@H](N)c1cccc(-c2cc(CCC3CCCO3)cc(COc3ccccc3CC(=O)O)c2)c1F]

The title compound was synthesized in the same manner as Example 114-B from tert-butyl 2-(2-((3′-((R)-1-((tert-butoxycarbonyl)amino)ethyl)-2′-fluoro-5-(2-(tetrahydrofuran-2-yl)ethyl)-[1,1′-biphenyl]-3-yl)methoxy)phenyl)acetate. ¹H NMR (400 MHz, METHANOL-d₄) δ ppm 7.68 (s, 1H) 7.52 (td, J=7.55, 1.65 Hz, 1H) 7.40-7.47 (m, 1H) 7.26-7.34 (m, 3H) 7.10-7.22 (m, 2H) 6.94 (d, J=7.95 Hz, 1H) 6.86 (td, J=7.40, 0.86 Hz, 1H) 5.16 (s, 2H) 4.72 (q, J=6.93 Hz, 1H) 3.81-3.91 (m, 2H) 3.68-3.77 (m, 1H) 3.57 (s, 2H) 2.67-2.88 (m, 2H) 1.98-2.09 (m, 1H) 1.76-1.98 (m, 4H) 1.67 (d, J=6.85 Hz, 3H) 1.47-1.60 (m, 1H). HRMS calcd. for C₂₉H₃₂FNO₄ (M−H)⁺ 476.2229. found 476.2237.

### Example 237

### (R)-2-(2-((3′-(1-aminoethyl)-5-(2-cyclopentylethyl)-2′-fluoro-[1,1′-biphenyl]-3-yl)methoxy)phenyl)acetic acid

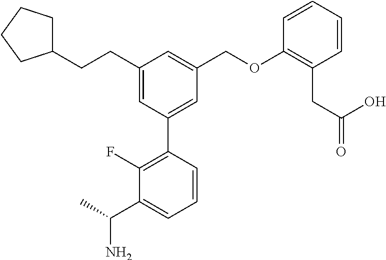 [SMILES: C[C@@H](N)c1cccc(-c2cc(CCC3CCCC3)cc(COc3ccccc3CC(=O)O)c2)c1F]

The title compound was synthesized in the same manner as Example 236 using cyclopentanecarbaldehyde in place of furan-2-carbaldehyde. ¹H NMR (400 MHz, DMSO-d₆) δ ppm 7.50-7.59 (m, 1H) 7.45 (s, 1H) 7.36 (td, J=7.52, 1.71 Hz, 1H) 7.17-7.32 (m, 5H) 7.03 (d, J=7.82 Hz, 1H) 6.85-6.92 (m, 1H) 5.15 (s, 2H) 4.34 (q, J=6.48 Hz, 1H) 3.54 (s, 2H) 2.62-2.69 (m, 2H) 1.70-1.82 (m, 3H) 1.53-1.67 (m, 4H) 1.43-1.53 (m, 2H) 1.33 (d, J=6.60 Hz, 3H) 1.08-1.19 (m, 2H). HRMS calcd. for C₃₀H₃₄FNO₃ (M+H)⁺ 476.2601. found 476.2609.

### Example 238

### 2-(2-((3′-((R)-1-aminoethyl)-2′-fluoro-5-(2-(pyrrolidin-2-yl)ethyl)-[1,1′-biphenyl]-3-yl)methoxy)phenyl)acetic acid

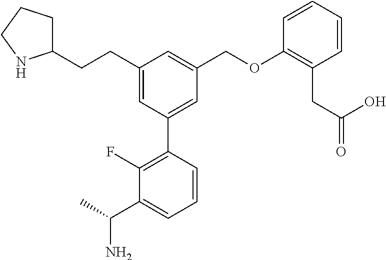 [SMILES: C[C@@H](N)c1cccc(-c2cc(CCC3CCCN3)cc(COc3ccccc3CC(=O)O)c2)c1F]

The title compound was synthesized in the same manner as Example 236 using tert-butyl 2-formylpyrrolidine-1-carboxylate in place of furan-2-carbaldehyde. ¹H NMR (400 MHz, CHLOROFORM-d) δ ppm 7.66 (s, 1H) 7.37-7.45 (m, 1H) 7.28-7.31 (m, 2H) 7.15-7.22 (m, 3H) 7.05-7.13 (m, 1H) 6.84 (t, J=7.27 Hz, 1H) 6.73 (d, J=8.19 Hz, 1H) 5.13-5.37 (m, 2H) 4.46 (q, J=6.72 Hz, 1H) 3.46-3.81 (m, 2H) 3.02-3.13 (m, 1H) 2.85-3.00 (m, 2H) 2.72-2.81 (m, 2H) 2.24 (d, J=5.26 Hz, 1H) 1.60-1.87 (m, 5H) 1.44-1.51 (m, 3H). HRMS calcd. for C₂₉H₃₃FN₂O₃(M+H)⁺ 477.2553. found 477.2550.

### Example 239

### Example 239-A

### (R)-tert-butyl 2-(2-((5-(2-(4-benzylmorpholin-2-yl)vinyl)-3′-(1-((tert-butoxycarbonyl)amino)ethyl)-2′-fluoro-[1,1′-biphenyl]-3-yl)methoxy)phenyl)acetate

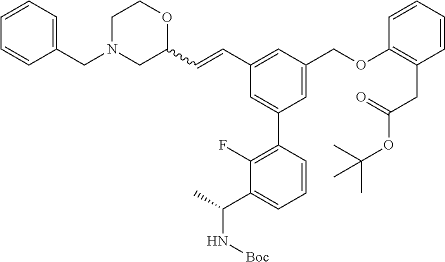 [SMILES: C[C@@H](NC(=O)OC(C)(C)C)c1cccc(-c2cc(C=CC3CN(Cc4ccccc4)CCO3)cc(COc3ccccc3CC(=O)OC(C)(C)C)c2)c1F]

The title compound was synthesized in the same manner as Example 141-A from tert-butyl 2-(2-((3-(2-(4-benzylmorpholin-2-yl)vinyl)-5-chlorobenzyl)oxy)phenyl)acetate (Intermediate 155) and (R)-tert-butyl (1-(2-fluoro-3-(4,4,5,5-tetramethyl-1,3,2-dioxaborolan-2-yl)phenyl)ethyl)carbamate (Intermediate 27-B); S-Phos palladacycle was used instead of PdCl₂(dppf).CH₂Cl₂ adduct in the coupling step. MS (ESI+) m/z 737.8 (M+H).

### Example 239-B

### tert-Butyl 2-(2-((3′-((R)-1-((tert-butoxycarbonyl)amino)ethyl)-2′-fluoro-5-(2-(morpholin-2-yl)ethyl)-[1,1′-biphenyl]-3-yl)methoxy)phenyl)acetate

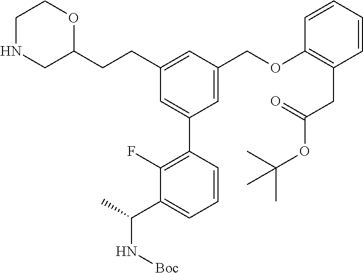 [SMILES: C[C@@H](NC(=O)OC(C)(C)C)c1cccc(-c2cc(CCC3CNCCO3)cc(COc3ccccc3CC(=O)OC(C)(C)C)c2)c1F]

To (R)-tert-butyl 2-(2-((5-(2-(4-benzylmorpholin-2-yl)vinyl)-3′-(1-((tert-butoxycarbonyl)amino)ethyl)-2′-fluoro-[1,1′-biphenyl]-3-yl)methoxy)phenyl)acetate (0.1 g, 0.136 mmol) in MeOH (2.7 mL) was added 1,1,2-trichloroethane (0.014 mL, 0.149 mmol) and then Pd-C (0.037 g, 0.035 mmol) and then the mixture was placed under vacuum and then backfilled with hydrogen from a balloon and stirred for 30 minutes and then filtered and concentrated to obtain the title compound. MS (ESI+) m/z 649.6 (M+H).

### Example 239-C

### 2-(2-((3′-((R)-1-aminoethyl)-2′-fluoro-5-(2-(morpholin-2-yl)ethyl)-[1,1′-biphenyl]-3-yl)methoxy)phenyl)acetic acid

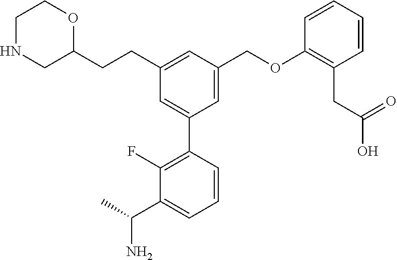 [SMILES: C[C@@H](N)c1cccc(-c2cc(CCC3CNCCO3)cc(COc3ccccc3CC(=O)O)c2)c1F]

The title compound was synthesized in the same manner as Example 114-B from tert-butyl 2-(2-((3′-((R)-1-((tert-butoxycarbonyl)amino)ethyl)-2′-fluoro-5-(2-(morpholin-2-yl)ethyl)-[1,1′-biphenyl]-3-yl)methoxy)phenyl)acetate. 1H NMR (400 MHz, CHLOROFORM-d) δ ppm 7.39-7.46 (m, 2H) 7.32-7.38 (m, 2H) 7.28-7.31 (m, 2H) 7.15-7.24 (m, 2H) 7.02 (d, J=8.07 Hz, 1H) 6.94 (td, J=7.40, 0.98 Hz, 1H) 4.91-5.16 (m, 2H) 4.47 (q, J=6.72 Hz, 1H) 3.74-3.87 (m, 2H) 3.46-3.68 (m, 3H) 3.16 (d, J=11.13 Hz, 1H) 2.81-3.01 (m, 3H) 2.69 (ddd, J=14.21, 7.73, 3.48 Hz, 2H) 2.48-2.58 (m, 1H) 1.83-1.98 (m, 1H) 1.72 (td, J=9.72, 3.91 Hz, 1H) 1.48 (d, J=6.60 Hz, 3H). HRMS calcd. for C₂₉H₃₃FN₂O₄(M−H)⁺ 491.2346. found 491.2334.

### Example 240

### Example 240-A

### (R)-tert-butyl 2-(2-((3′-(1-((tert-butoxycarbonyl)amino)ethyl)-2′-fluoro-5-(2-(1-methylpyrrolidin-2-yl)vinyl)-[1,1′-biphenyl]-3-yl)methoxy)phenyl)acetate

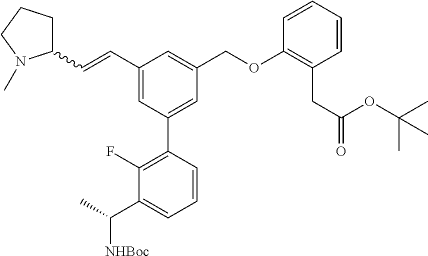 [SMILES: C[C@@H](NC(=O)OC(C)(C)C)c1cccc(-c2cc(C=CC3CCCN3C)cc(COc3ccccc3CC(=O)OC(C)(C)C)c2)c1F]

The title compound was synthesized in the same manner as Example 141-A from tert-butyl 2-(2-((3-bromo-5-(2-(1-methylpyrrolidin-2-yl)vinyl)benzyl)oxy)phenyl)acetate (Intermediate 159) and (R)-tert-butyl(1-(2-fluoro-3-(4,4,5,5-tetramethyl-1,3,2-dioxaborolan-2-yl)phenyl)ethyl)carbamate (Intermediate 27-B). MS (ESI+) m/z 645.3 (M+H).

### Example 240-B

### 2-(2-((3′-((R)-1-aminoethyl)-2′-fluoro-5-(2-(1-methylpyrrolidin-2-yl)ethyl)-[1,1′-biphenyl]-3-yl)methoxy)phenyl)acetic acid

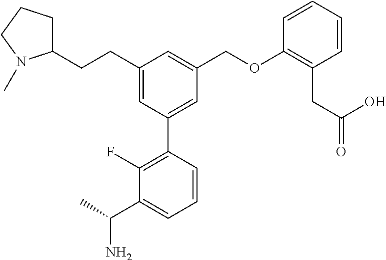 [SMILES: C[C@@H](N)c1cccc(-c2cc(CCC3CCCN3C)cc(COc3ccccc3CC(=O)O)c2)c1F]

The title compound was synthesized in the same manner as Example 240. ¹H NMR (400 MHz, DMSO-d₆) δ ppm 7.51-7.58 (m, 1H) 7.44 (s, 1H) 7.32-7.39 (m, 2H) 7.15-7.31 (m, 4H) 7.02 (d, J=7.95 Hz, 1H) 6.84-6.90 (m, 1H) 5.16 (s, 2H) 4.34 (q, J=6.64 Hz, 1H) 3.52 (s, 2H) 2.93-3.01 (m, 1H) 2.58-2.74 (m, 2H) 2.24 (s, 3H) 2.10-2.19 (m, 2H) 1.86-2.04 (m, 2H) 1.60-1.71 (m, 2H) 1.42-1.58 (m, 2H) 1.32 (d, J=6.60 Hz, 3H). HRMS calcd. for C₃₀H₃₅FN₂O₃(M−H)⁺ 489.2553. found 489.2556.

### Example 241

### Example 241-A

### Tert-Butyl 2-(2-((3-chloro-5-(piperidin-1-yl)benzyl)oxy)phenyl)acetate

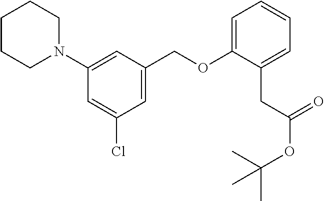 [SMILES: CC(C)(C)OC(=O)Cc1ccccc1OCc1cc(Cl)cc(N2CCCCC2)c1]

The title compound was synthesized in the same manner as Example 226-A from tert-Butyl 2-(2-((3-bromo-5-chlorobenzyl)oxy)phenyl)acetate (Intermediate 53) and piperidine. MS (ESI+) m/z 416.2, 418.2 (M+H).

### Example 241-B

### (R)-tert-butyl 2-(2-((3′-(1-((tert-butoxycarbonyl)amino)ethyl)-2′-fluoro-5-(piperidin-1-yl)-[1,1′-biphenyl]-3-yl)methoxy)phenyl)acetate

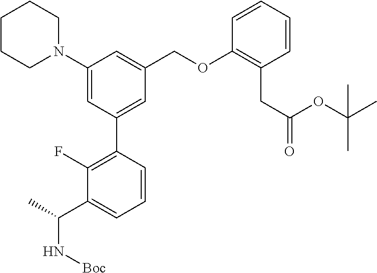 [SMILES: C[C@@H](NC(=O)OC(C)(C)C)c1cccc(-c2cc(COc3ccccc3CC(=O)OC(C)(C)C)cc(N3CCCCC3)c2)c1F]

The title compound was synthesized in the same manner as Example 141-A from tert-butyl 2-(2-((3-chloro-5-(piperidin-1-yl)benzyl)oxy)phenyl)acetate and from Intermediate 27-B. MS (ESI+) m/z 619.5 (M+H). S-Phos palladacycle was used instead of PdCl₂(dppf).CH₂Cl₂ adduct in the coupling step.

### Example 241-C

### (R)-2-(2-((3′-(1-Aminoethyl)-2′-fluoro-5-(piperidin-1-yl)-[1,1′-biphenyl]-3-yl)methoxy)phenyl)acetic acid

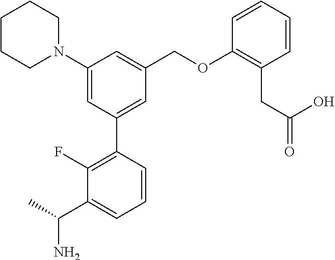 [SMILES: C[C@@H](N)c1cccc(-c2cc(COc3ccccc3CC(=O)O)cc(N3CCCCC3)c2)c1F]

The title compound was synthesized in the same manner as Example 114-B from (R)-tert-butyl 2-(2-((3′-(1-((tert-butoxycarbonyl)amino)ethyl)-2′-fluoro-5-(piperidin-1-yl)-[1,1′-biphenyl]-3-yl)methoxy)phenyl)acetate. ¹H NMR (400 MHz, DMSO-d₆) δ ppm 7.49-7.57 (m, 1H) 7.36 (td, J=7.55, 1.77 Hz, 1H) 7.15-7.26 (m, 3H) 6.98-7.07 (m, 3H) 6.94 (s, 1H) 6.88 (td, J=7.40, 0.86 Hz, 1H) 5.11 (s, 2H) 4.36 (d, J=6.60 Hz, 1H) 3.55 (s, 2H) 3.16-3.24 (m, 4H) 1.50-1.68 (m, 6H) 1.33 (d, J=6.72 Hz, 3H). HRMS calcd. for C₂₈H₃₁FN₂O₃(M+H)⁺ 463.2397. found 463.2401.

### Example 242

### Example 242-A

### tert-Butyl 2-(2-((3-chloro-5-((cyclopropylmethyl)(ethyl)amino)benzyl)oxy)phenyl)acetate

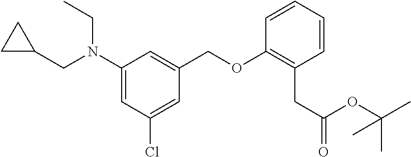 [SMILES: CCN(CC1CC1)c1cc(Cl)cc(COc2ccccc2CC(=O)OC(C)(C)C)c1]

To tert-Butyl 2-(2-((3-chloro-5-((cyclopropylmethyl)amino)benzyl)oxy)phenyl)acetate (Example 94-A) (0.13 g, 0.323 mmol) in DCE (1.6 mL) was added acetic acid (0.037 mL, 0.647 mmol) followed by acetaldehyde (0.036 mL, 0.647 mmol) and stirred at room temperature for 1 hour. Sodium triacetoxyborohydride (0.103 g, 0.485 mmol) was then added and continued stirring at room temperature. The reaction was diluted with dcm:water (3 mL:3 mL) after 30 minutes. Then added a saturated solution of sodium bicarbonate, the layers were separated and the organic layer was dried, concentrated and absorbed onto silica to purify via fcc (0-40% EA:heptanes) to obtain the title compound. MS (ESI+) m/z 430.2, 432.2 (M+H).

### Example 242-B

### B. (R)-tert-butyl 2-(2-((3′-(1-((tert-butoxycarbonyl)amino)ethyl)-5-((cyclopropylmethyl)(ethyl)amino)-2′-fluoro-[1,1′-biphenyl]-3-yl)methoxy)phenyl)acetate

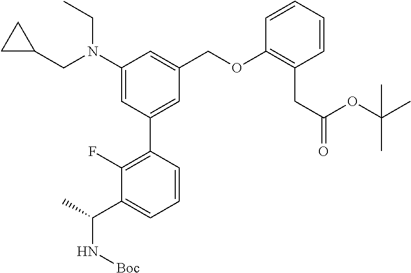 [SMILES: CCN(CC1CC1)c1cc(COc2ccccc2CC(=O)OC(C)(C)C)cc(-c2cccc([C@@H](C)NC(=O)OC(C)(C)C)c2F)c1]

The title compound was synthesized in the same manner as Example 141-A from tert-butyl 2-(2-((3-chloro-5-((cyclopropylmethyl)(ethyl)amino)benzyl)oxy)phenyl)acetate and Intermediate 27-B. MS (ESI+) m/z 633.5 (M+H). S-Phos palladacycle was used instead of PdCl₂(dppf).CH₂Cl₂ adduct in the coupling step.

### Example 242-C

### (R)-2-(2-((3′-(1-aminoethyl)-5-((cyclopropylmethyl)(ethyl)amino)-2′-fluoro-[1,1′-biphenyl]-3-yl)methoxy)phenyl)acetic acid

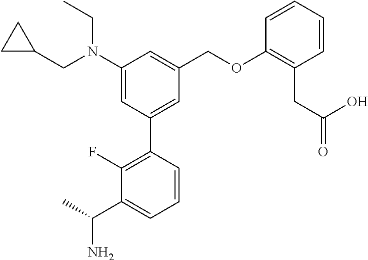 [SMILES: CCN(CC1CC1)c1cc(COc2ccccc2CC(=O)O)cc(-c2cccc([C@@H](C)N)c2F)c1]

The title compound was synthesized in the same manner as Example 114-B from (R)-tert-butyl 2-(2-((3′-(1-((tert-butoxycarbonyl)amino)ethyl)-5-((cyclopropylmethyl)(ethyl) amino)-2′-fluoro-[1,1′-biphenyl]-3-yl)methoxy)phenyl)acetate. 1H NMR (400 MHz, DMSO-d₆) δ ppm 7.49-7.56 (m, 1H) 7.34 (td, J=7.46, 1.71 Hz, 1H) 7.15-7.26 (m, 3H) 7.03 (d, J=7.83 Hz, 1H) 6.84-6.91 (m, 1H) 6.82 (s, 2H) 6.71 (s, 1H) 5.10 (s, 2H) 4.35 (d, J=6.60 Hz, 1H) 3.56 (s, 2H) 3.46 (q, J=6.93 Hz, 2H) 3.21 (d, J=6.24 Hz, 2H) 1.32 (d, J=6.72 Hz, 3H) 0.98-1.15 (m, 4H) 0.42-0.52 (m, 2H) 0.21-0.29 (m, 2H). HRMS calcd. for C₂₉H₃₃FN₂O₃(M−H)⁺ 475.2397. found 475.2404.

### Example 243

### Example 243-A

### tert-butyl 2-(2-((5-chloro-2′,3′,4′,5′-tetrahydro-[1,1′-biphenyl]-3-yl)methoxy)phenyl)acetate

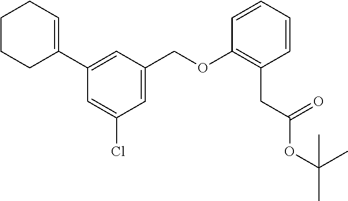 [SMILES: CC(C)(C)OC(=O)Cc1ccccc1OCc1cc(Cl)cc(C2=CCCCC2)c1]

The title compound was synthesized in the same manner as Example 141-A from Intermediate 53 tert-Butyl 2-(2-((3-bromo-5-chlorobenzyl)oxy)phenyl)acetate and 2-(cyclohex-1-en-1-yl)-4,4,5,5-tetramethyl-1,3,2-dioxaborolane (CAS #141091-37-4). MS (ESI+) m/z 357.3, 359.2 (M−t-butyl)⁺.

### Example 243-B

### (R)-tert-butyl 2-(2-((3″-(1-((tert-butoxycarbonyl)amino)ethyl)-2″-fluoro-2,3,4,5-tetrahydro-[1,1′:3′,1″-terphenyl]-5′-yl)methoxy)phenyl)acetate

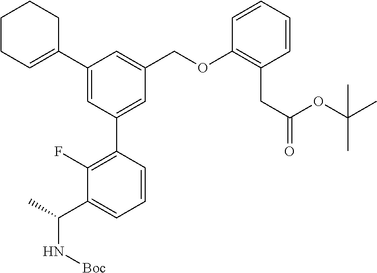 [SMILES: C[C@@H](NC(=O)OC(C)(C)C)c1cccc(-c2cc(COc3ccccc3CC(=O)OC(C)(C)C)cc(C3=CCCCC3)c2)c1F]

The title compound was synthesized in the same manner as Example 141-A from tert-butyl 2-(2-((5-chloro-2′,3′,4′,5′-tetrahydro-[1,1′-biphenyl]-3-yl)methoxy)phenyl)acetate and from Intermediate 27-B. MS (ESI+) m/z 516.4 (M−Boc)⁺. S-Phos palladacycle was used instead of PdCl₂(dppf).CH₂Cl₂ adduct in the coupling step.

### Example 243-C

### (R)-tert-Butyl 2-(2-((3′-(1-((tert-butoxycarbonyl)amino)ethyl)-5-cyclohexyl-2′-fluoro-[1,1′-biphenyl]-3-yl)methoxy)phenyl)acetate

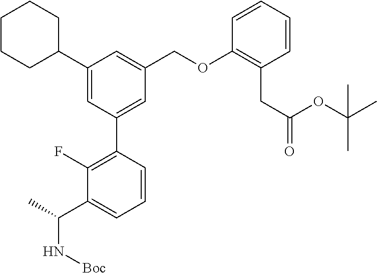 [SMILES: C[C@@H](NC(=O)OC(C)(C)C)c1cccc(-c2cc(COc3ccccc3CC(=O)OC(C)(C)C)cc(C3CCCCC3)c2)c1F]

The title compound was synthesized in the same manner as Example 236-B from (R)-tert-butyl 2-(2-((3″-(1-((tert-butoxycarbonyl)amino)ethyl)-2″-fluoro-2,3,4,5-tetrahydro-[1,1′:3′,1″-terphenyl]-5′-yl)methoxy)phenyl)acetate. MS (ESI+) m/z 462.3 (M−Boc-t-butyl)⁺.

### Example 243-D

### (R)-2-(2-((3′-(1-aminoethyl)-5-cyclohexyl-2′-fluoro-[1,1′-biphenyl]-3-yl)methoxy)phenyl)acetic acid

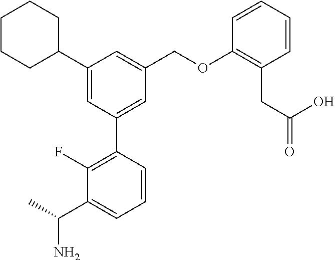 [SMILES: C[C@@H](N)c1cccc(-c2cc(COc3ccccc3CC(=O)O)cc(C3CCCCC3)c2)c1F]

The title compound was synthesized in the same manner as Example 114-B from (R)-tert-butyl 2-(2-((3′-(1-((tert-butoxycarbonyl)amino)ethyl)-5-cyclohexyl-2′-fluoro-[1,1′-biphenyl]-3-yl)methoxy)phenyl)acetate. 1H NMR (400 MHz, METHANOL-d₄) δ ppm 7.65 (s, 1H) 7.50 (td, J=7.55, 1.65 Hz, 1H) 7.40-7.46 (m, 1H) 7.24-7.33 (m, 3H) 7.19 (dd, J=7.46, 1.59 Hz, 1H) 7.14 (td, J=7.79, 1.77 Hz, 1H) 6.95 (d, J=7.34 Hz, 1H) 6.86 (td, J=7.40, 0.98 Hz, 1H) 5.15 (s, 2H) 4.69 (q, J=6.85 Hz, 1H) 3.57 (s, 2H) 2.56-2.66 (m, 1H) 1.83-1.96 (m, 4H) 1.77 (d, J=12.47 Hz, 1H) 1.65 (d, J=6.85 Hz, 3H) 1.40-1.59 (m, 4H) 1.26-1.39 (m, 1H). HRMS calcd. for C₂₉H₃₂FNO₃ (M+H)⁺ 462.2444. found 462.2459.

### Example 244

### (R)-2-(2-((3′-(1-aminoethyl)-5-cyclopentyl-2′-fluoro-[1,1′-biphenyl]-3-yl)methoxy)phenyl)acetic acid

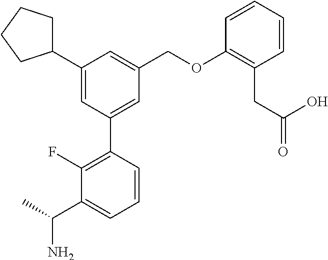 [SMILES: C[C@@H](N)c1cccc(-c2cc(COc3ccccc3CC(=O)O)cc(C3CCCC3)c2)c1F]

The title compound was synthesized in the same manner as Example 243. ¹H NMR (400 MHz, DMSO-d₆) δ ppm 7.50-7.60 (m, 1H) 7.45 (s, 1H) 7.29-7.39 (m, 3H) 7.14-7.28 (m, 3H) 7.04 (d, J=7.82 Hz, 1H) 6.89 (td, J=7.37, 1.04 Hz, 1H) 5.15 (s, 2H) 4.34 (q, J=6.85 Hz, 1H) 2.98-3.11 (m, 1H) 3.54 (s, 2H) 2.00-2.12 (m, 2H) 1.74-1.85 (m, 2H) 1.52-1.72 (m, 4H) 1.32 (d, J=6.60 Hz, 3H). HRMS calcd. for C₂₈H₃₀FNO₃ (M+H)⁺ 448.2288. found 448.2269.

### Example 245

### (R)-2-(2-((3′-(1-aminoethyl)-5-((cyclopropylmethyl)(methyl)amino)-2′-fluoro-[1,1′-biphenyl]-3-yl)methoxy)phenyl)acetic acid

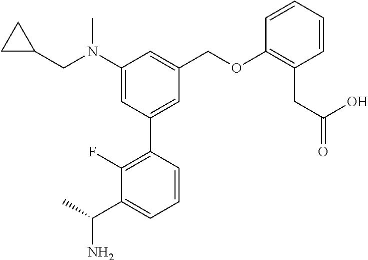 [SMILES: C[C@@H](N)c1cccc(-c2cc(COc3ccccc3CC(=O)O)cc(N(C)CC3CC3)c2)c1F]

The title compound was synthesized in the same manner as Example 242. ¹H NMR (400 MHz, DMSO-d₆) δ 7.47-7.61 (m, 2H) 7.30-7.38 (m, 1H) 7.17-7.25 (m, 2H) 7.04 (d, J=7.82 Hz, 1H) 6.82-6.93 (m, 3H) 6.78 (s, 1H) 5.11 (s, 2H) 4.59-4.68 (m, 1H) 3.58 (s, 2H) 3.28 (d, J=6.36 Hz, 2H) 2.98 (s, 3H) 1.52 (d, J=6.72 Hz, 3H) 0.94-1.07 (m, 1H) 0.40-0.48 (m, 2H) 0.20-0.27 (m, 2H). HRMS calcd. for C₂₈H₃₁FN₂O₃(M+H)⁺ 463.2397. found 463.2398.

### Intermediate 246

### Intermediate 246-A

### tert-Butyl 2-(2-((3-bromo-5-((dihydro-2H-pyran-4(3H)-ylidene)methyl)benzyl)oxy)phenyl)acetate

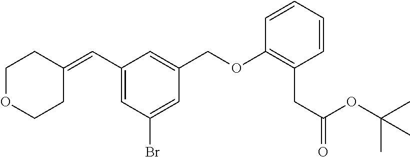 [SMILES: CC(C)(C)OC(=O)Cc1ccccc1OCc1cc(Br)cc(C=C2CCOCC2)c1]

To a mixture of tert-butyl 2-(2-((3-bromo-5-((triphenylphosphoranyl)methyl)benzyl)oxy)phenyl)acetate hydrobromide (Intermediate 152) (0.11 g, 0.150 mmol) in EtOH (1.498 ml) was added dihydro-2H-pyran-4(3H)-one (CAS #29943-42-8) (0.017 mL, 0.180 mmol) and sodium ethoxide (0.0138 g, 0.195 mmol) and heated to 80° C., further dihydro-2H-pyran-4(3H)-one (3 eq) was added after 4 h and the reaction left stirring overnight at same temperature. At this point, NaOH (5 mg, 0.125 mmol) was added. The reaction was cooled to room temperature after 1 hour. The solution was diluted with EA and water and the EA layer was removed, dried, concentrated and absorbed onto silica to purify via fcc (0-50% EA:heptanes) to the title compound. MS (ESI+) m/z 417.15, 419.11 (M−t-butyl)⁺.

### Example 246-B

### (R)-tert-butyl 2-(2-((3′-(1-((tert-butoxycarbonyl)amino)ethyl)-5-((dihydro-2H-pyran-4(3H)-ylidene)methyl)-2′-fluoro-[1,1′-biphenyl]-3-yl)methoxy)phenyl)acetate

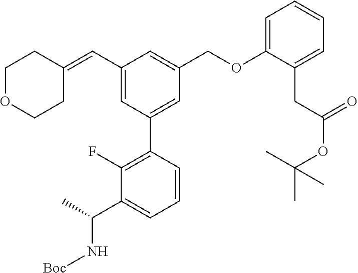 [SMILES: C[C@@H](NC(=O)OC(C)(C)C)c1cccc(-c2cc(C=C3CCOCC3)cc(COc3ccccc3CC(=O)OC(C)(C)C)c2)c1F]

The title compound was synthesized in the same manner as Example 141-A from tert-butyl 2-(2-((3-bromo-5-((dihydro-2H-pyran-4(3H)-ylidene)methyl)benzyl)oxy)phenyl)acetate and from Intermediate 27-B. MS (ESI+) m/z 632.5 (M+H).

### Example 246-C

### (R)-2-(2-((3′-(1-aminoethyl)-2′-fluoro-5-((tetrahydro-2H-pyran-4-yl)methyl)-[1,1′-biphenyl]-3-yl)methoxy)phenyl)acetic acid

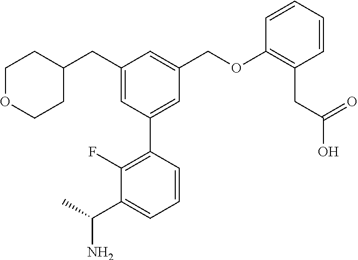 [SMILES: C[C@@H](N)c1cccc(-c2cc(COc3ccccc3CC(=O)O)cc(CC3CCOCC3)c2)c1F]

The title compound was synthesized in the same manner as Example 236. ¹H NMR (400 MHz, DMSO-d6) 7.51-7.59 (m, 1H) 7.47 (s, 1H) 7.36 (td, J=7.52, 1.71 Hz, 1H) 7.16-7.29 (m, 5H) 7.02 (d, J=8.07 Hz, 1H) 6.84-6.91 (m, 1H) 5.16 (s, 2H) 4.34 (d, J=6.60 Hz, 1H) 3.80 (dd, J=11.25, 2.81 Hz, 2H) 3.54 (s, 2H) 3.18-3.28 (m, 2H) 2.58 (d, J=7.09 Hz, 2H) 1.70-1.83 (m, 1H) 1.50 (d, J=11.13 Hz, 2H) 1.32 (d, J=6.60 Hz, 3H) 1.15-1.28 (m, 2H). HRMS calcd. for C₂₉H₃₂FNO₄ (M+H)⁺ 478.2394. found 478.2386.

### Example 247

### (R)-2-(2-((3′-(1-aminoethyl)-5-(azetidin-1-yl)-2′-fluoro-[1,1′-biphenyl]-3-yl)methoxy)phenyl)acetic acid

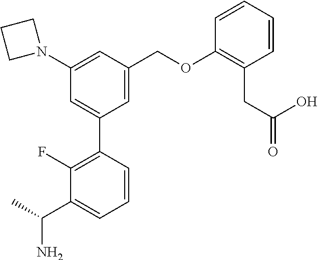 [SMILES: C[C@@H](N)c1cccc(-c2cc(COc3ccccc3CC(=O)O)cc(N3CCC3)c2)c1F]

The title compound was synthesized in the same manner as Example 241. ¹H NMR (400 MHz, DMSO-d₆) 7.53 (td, J=7.27, 1.59 Hz, 1H) 7.29-7.37 (m, 1H) 7.15-7.26 (m, 3H) 7.01 (d, J=7.95 Hz, 1H) 6.83-6.95 (m, 2H) 6.53 (s, 1H) 6.43 (s, 1H) 5.09 (s, 2H) 4.34 (q, J=6.60 Hz, 1H) 3.84 (t, J=7.15 Hz, 4H) 3.54 (s, 2H) 2.31 (quin, J=7.18 Hz, 2H) 1.32 (d, J=6.60 Hz, 3H). HRMS calcd. for C₂₆H₂₇FN₂O₃(M+H)⁺ 435.2084. found 435.2082.

### Example 248

### (R)-2-(2-((3′-(1-aminoethyl)-2′-fluoro-5-(2-(tetrahydro-2H-pyran-4-yl)ethyl)-[1,1′-biphenyl]-3-yl)methoxy)phenyl)acetic acid

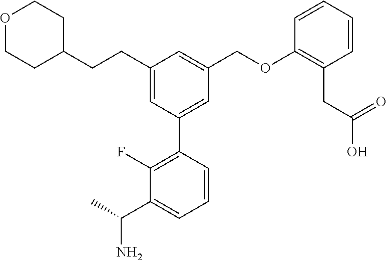 [SMILES: C[C@@H](N)c1cccc(-c2cc(CCC3CCOCC3)cc(COc3ccccc3CC(=O)O)c2)c1F]

The title compound was synthesized in the same manner as Example 246. ¹H NMR (400 MHz, DMSO-d₆) 7.51-7.60 (m, 1H) 7.46 (s, 1H) 7.16-7.39 (m, 6H) 7.02 (d, J=7.82 Hz, 1H) 6.85-6.92 (m, 1H) 5.16 (s, 2H) 4.34 (q, J=6.68 Hz, 1H) 3.82 (dd, J=11.37, 2.81 Hz, 2H) 3.54 (s, 2H) 3.24 (td, J=11.65, 1.90 Hz, 2H) 2.63-2.72 (m, 2H) 1.43-1.68 (m, 5H) 1.27-1.36 (m, 3H) 1.12-1.26 (m, 2H). HRMS calcd. for C₃₀H₃₄FNO₄ (M+H)⁺ 492.2550. found 492.2550.

### Example 249

### Ethyl 2-(2-((3′-((R)-1-aminoethyl)-2′-fluoro-5-((((R)-tetrahydrofuran-2-yl)methyl)amino)-[1,1′-biphenyl]-3-yl)methoxy)phenyl)acetate

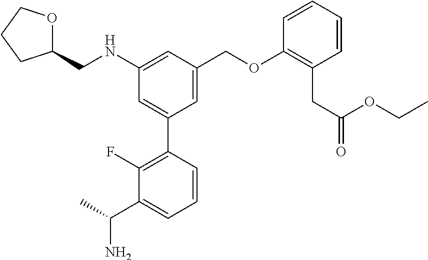 [SMILES: CCOC(=O)Cc1ccccc1OCc1cc(NC[C@H]2CCCO2)cc(-c2cccc([C@@H](C)N)c2F)c1]

To 2-(2-((3′-((R)-1-Aminoethyl)-2′-fluoro-5-((((R)-tetrahydrofuran-2-yl)methyl)amino)-[1,1′-biphenyl]-3-yl)methoxy)phenyl)acetic acid (Example 153) (0.055 g, 0.115 mmol) in EtOH (1.2 mL) was added sulfuric acid (50 μL) and the reaction was heated at 80° C. The reaction was concentrated after 3 hours, diluted with EA and washed with a saturated aq. solution of sodium bicarbonate and then diluted with water. The EA layer was removed, dried, concentrated and absorbed onto silica to purify via fcc (0-20% MeOH containing 10% NH4OH:DCM) to obtain the title compound. ¹H NMR (400 MHz, DMSO-d₆) δ ppm 7.54 (td, J=7.15, 1.83 Hz, 1H) 7.15-7.30 (m, 4H) 7.05 (d, J=7.58 Hz, 1H) 6.89 (td, J=7.40, 0.98 Hz, 1H) 6.63-6.75 (m, 3H) 5.75 (t, J=5.87 Hz, 1H) 5.02 (s, 2H) 4.30 (q, J=6.60 Hz, 1H) 3.88-4.05 (m, 3H) 3.74-3.83 (m, 1H) 3.58-3.68 (m, 3H) 3.05-3.18 (m, 2H) 1.74-2.01 (m, 3H) 1.52-1.64 (m, 1H) 1.28 (d, J=6.60 Hz, 3H) 0.98-1.05 (m, 3H). HRMS calcd. for C₃₀H₃₅FN₂O₄(M+H)⁺ 507.2659. found 507.2654.

### Example 250

### Example 250-A

### (3-Bromo-5-(methylsulfonyl)phenyl)methanol

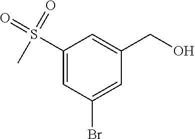

(3-bromo-5-iodophenyl)methanol (CAS #18813-08-3) (1000 mg, 3.2 mmol), copper(I) trifluoromethanesulfonate benzene complex (CAS #42152-46-5) (80 mg, 0.160 mmol) and methanesulfinic acid sodium salt (CAS #20277-69-4) (427 mg, 4.19 mmol) were mixed together in a reaction vial; a stream of N₂(g) was blown over the top of the reaction for 5.0 min; then the vial was sealed with a septa cap and N,N-dimethyethylenediamine (CAS #110-70-3) (28.2 mg, 0.320 mmol) was added via syringe followed by DMSO (3.0 mL); this mixture was then heated at 110° C. for 16 hr and then cooled to r.t. The reaction was diluted with EtOAc/H₂O and the layers were filtered over a Celite pad and then partitioned. The organic layer was washed with brine; dried over sodium sulfate and concentrated in vacuo to give the crude product. This material was purified by flash column chromatography on silica gel (heptanes/EtOAc=100:0 to 0:100) to afford the title compound. ¹H NMR (400 MHz, CHLOROFORM-d) δ ppm 8.18 (br.s, 1H) 7.98-8.02 (m, 1H) 7.86-7.92 (m, 1H) 7.81 (br. s, 1H) 4.80 (s, 2H) 3.07 (s, 3H).

### Example 250-B

### tert-Butyl 2-(2-((3-bromo-5-(methylsulfonyl)benzyl)oxy)phenyl)acetate

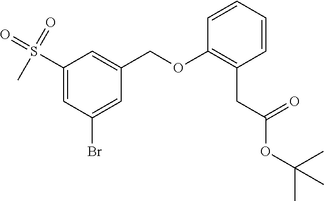 [SMILES: CC(C)(C)OC(=O)Cc1ccccc1OCc1cc(Br)cc(S(C)(=O)=O)c1]

The title compound was synthesized as described in Intermediate 55 starting with (3-bromo-5-(methylsulfonyl)phenyl)methanol. ¹H NMR (400 MHz, CHLOROFORM-d) δ ppm 7.88-8.27 (m, 3H) 7.19-7.25 (m, 2H) 6.93-7.01 (m, 1H) 6.86 (d, J=7.58 Hz, 1H) 5.14 (s, 2H) 3.61 (s, 2H) 3.10 (s, 3H) 1.40 (s, 9H).

### Example 250-C

### (R)-tert-Butyl 2-(2-((3′-(1-((tert-butoxycarbonyl)amino)ethyl)-2′-fluoro-5-(methylsulfonyl)-[1,1′-biphenyl]-3-yl)methoxy)phenyl)acetate

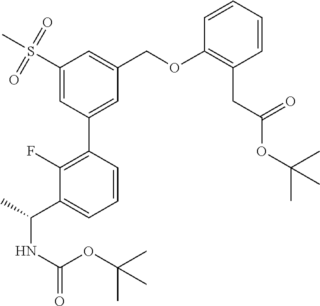 [SMILES: C[C@@H](NC(=O)OC(C)(C)C)c1cccc(-c2cc(COc3ccccc3CC(=O)OC(C)(C)C)cc(S(C)(=O)=O)c2)c1F]

To a solution of tert-Butyl 2-(2-((3-bromo-5-(methylsulfonyl)benzyl)oxy)phenyl)acetate (105 mg, 0.231 mmol) and (R)-tert-butyl (1-(2-fluoro-3-(4,4,5,5-tetramethyl-1,3,2-dioxaborolan-2-yl)phenyl)ethyl)carbamate (Intermediate 27-B) (93 mg, 0.254 mmol) in DMF (2.0 mL) was added a 2.0M solution of K₃PO₄ (0.519 mL, 1.038 mmol); this mixture was degassed for 10 minutes with N₂(g), and then PdCl₂(dppf).CH₂Cl₂ adduct (9.41 mg, 0.012 mmol) added. The reaction was sealed and heated at 110 C in an oil bath for 1 hr. The reaction was cooled to room temperature, filtered over a plug of Celite and the filtrate was partitioned between EtOAc/H₂O and the layers separated; the organic phase was brine washed, dried over Na₂SO₄, and concentrated in vacuo. The residue was purified by flash column chromatography on silica gel (heptanes/EtOAc=100:0 to 40:60) to afford the title compound. MS (ESI−) m/z 612.5 (M−H).

### Example 250-D

### (R)-2-(2-((3′-(1-aminoethyl)-2′-fluoro-5-(methylsulfonyl)-[1,1′-biphenyl]-3-yl)methoxy)phenyl)acetic acid

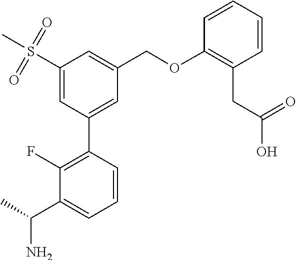 [SMILES: C[C@@H](N)c1cccc(-c2cc(COc3ccccc3CC(=O)O)cc(S(C)(=O)=O)c2)c1F]

To a solution of (R)-tert-Butyl 2-(2-((3′-(1-((tert-butoxycarbonyl)amino)ethyl)-2′-fluoro-5-(methylsulfonyl)-[1,1′-biphenyl]-3-yl)methoxy)phenyl)acetate (80 mg, 0.130 mmol) in dichloromethane (1.0 mL) was added 4.0M HCl in dioxane (5.0 mL). This mixture was stirred for several hours and then concentrated in vacuo. The resulting residue was purified by RP-HPLC (method B) to afford the title compound. 1H NMR (400 MHz, METHANOL-d4) δ ppm 8.27 (s, 1H) 8.03 (d, J=9.60 Hz, 2H) 7.63 (td, J=7.55, 1.71 Hz, 1H) 7.50-7.57 (m, 1H) 7.35-7.42 (m, 1H) 7.12-7.23 (m, 2H) 6.98 (d, J=7.58 Hz, 1H) 6.89 (td, J=7.39, 1.01 Hz, 1H) 5.31 (s, 2H) 4.70-4.78 (m, 1H) 3.57 (s, 2H) 3.21 (s, 3H) 1.69 (d, J=6.95 Hz, 3H). HRMS calcd. for C₂₄H₂₄FNO₅S (M+H)⁺ 458.1393. found 458.1413.

### Example 251

### (R)-2-(2-((3′-(1-aminoethyl)-5-(methylsulfonyl)-[1,1′-biphenyl]-3-yl)methoxy)phenyl)acetic acid

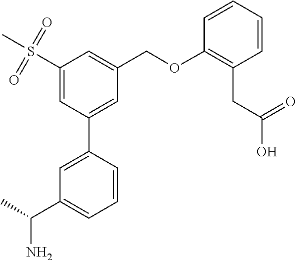 [SMILES: C[C@@H](N)c1cccc(-c2cc(COc3ccccc3CC(=O)O)cc(S(C)(=O)=O)c2)c1]

The title compound was synthesized in the same manner as Example 250. ¹H NMR (400 MHz, METHANOL-d4) δ ppm 8.44 (s, 1H) 8.28 (t, J=1.64 Hz, 1H) 8.17 (t, J=1.64 Hz, 1H) 7.97 (s, 1H) 7.74-7.81 (m, 1H) 7.52 (t, J=7.71 Hz, 1H) 7.41 (d, J=7.71 Hz, 1H) 7.15-7.23 (m, 2H) 6.99 (d, J=7.96 Hz, 1H) 6.90 (td, J=7.39, 0.88 Hz, 1H) 5.30 (d, J=3.03 Hz, 2H) 4.50 (q, J=6.91 Hz, 1H) 3.50-3.70 (m, 2H) 3.21 (s, 3H) 1.68 (d, J=6.95 Hz, 3H). HRMS calcd. for C₂₄H₂₅NO₅S (M+H)⁺ 440.1487. found 440.1502.

### Example 252

### (S)-2-(2-((3′-(1-aminoethyl)-2′-fluoro-5-(methylsulfonyl)-[1,1′-biphenyl]-3-yl)methoxy)phenyl)acetic acid

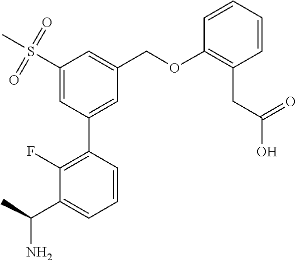 [SMILES: C[C@H](N)c1cccc(-c2cc(COc3ccccc3CC(=O)O)cc(S(C)(=O)=O)c2)c1F]

The title compound was synthesized in the same manner as Example 250. ¹H NMR (400 MHz, METHANOL-d4) δ ppm 8.26 (s, 1H) 8.03 (d, J=9.60 Hz, 2H) 7.63 (td, J=7.58, 1.64 Hz, 1H) 7.46-7.57 (m, 1H) 7.38 (t, J=7.77 Hz, 1H) 7.13-7.28 (m, 2H) 6.98 (d, J=8.08 Hz, 1H) 6.89 (td, J=7.42, 0.95 Hz, 1H) 5.30 (s, 2H) 4.72-4.78 (m, 1H) 3.57 (s, 2H) 3.21 (s, 3H) 1.69 (d, J=6.82 Hz, 3H) HRMS calcd. for C₂₄H₂₄FNO₅S (M+H)⁺ 458.1393. found 458.1410.

### Example 253

### Example 253-A

### (6-bromobenzo[d][1,3]dioxol-4-yl)methanol

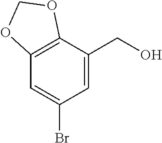 [SMILES: OCc1cc(Br)cc2c1OCO2]

To a solution of methyl 6-bromobenzo[d][1,3]dioxole-4-carboxylate (CAS #33842-18-1) (1.0 g, 4.05 mmol) in THF (25.0 mL) under nitrogen was added LiBH₄ (0.551 g, 25.31 mmol) and methanol (1.03 mL, 25.31 mmol). The reaction was stirred at r.t. for 3 days after which the reaction was carefully diluted with water, saturated brine, and EtOAc. The layers were separated and the aqueous layer was extracted with EtOAc, dried over Na₂SO₄, filtered and concentrated. The crude product was purified by flash chromatography (0-100% EtOAc:Heptanes) to provide the title compound. ¹H NMR (600 MHz, CHLOROFORM-d₂) δ ppm 7.06 (d, J=1.65 Hz, 1H) 6.94 (d, J=1.74 Hz, 1H) 6.03 (s, 2H) 4.68 (s, 2H) 1.68 (br.s., 1H).

### Example 253-B

### 6-bromo-4-(bromomethyl)benzo[d][1,3]dioxole

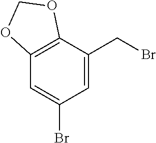 [SMILES: BrCc1cc(Br)cc2c1OCO2]

To a solution of (6-bromobenzo[d][1,3]dioxol-4-yl)methanol (860 mg, 3.72 mmol) was added tetrabromomethane (CAS #558-13-4) and triphenylphosphine at O° C. The reaction was then warmed to r.t and stirred overnight. The reaction was concentrated and taken up in EtOAc/H₂O and partitioned; the organic phase was washed with brine, dried over Na₂SO₄ and concentrated in vacuo. The crude material was purified using flash column chromatography chromatography on silica gel (heptanes/EtOAc=100:0 to 75:25) to afford the title compound. ¹H NMR (400 MHz, CHLOROFORM-d) δ ppm 6.99 (d, J=1.77 Hz, 1H) 6.89 (d, J=1.77 Hz, 1H) 6.04 (s, 2H) 4.38 (s, 2H).

### Example 253-C

### Tert-Butyl 2-(2-((6-bromobenzo[d][1,3]dioxol-4-yl)methoxy)phenyl)acetate

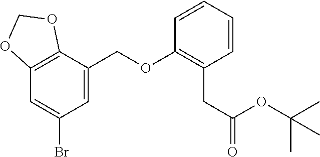 [SMILES: CC(C)(C)OC(=O)Cc1ccccc1OCc1cc(Br)cc2c1OCO2]

To a solution of 6-bromo-4-(bromomethyl)benzo[d][1,3]dioxole (1.0 g, 3.43 mmol) and tert-Butyl 2-(2-hydroxyphenyl)acetate (Intermediate 21) (0.650 g, 3.12 mmol) in DMF (15.0 mL) at room temperature was added potassium carbonate (0.539 g, 3.90 mmol) and potassium iodide (CAS #7681-11-0) (51 mg, 0.312 mmol). The mixture was stirred at room temperature overnight. The reaction mixture was partitioned between EtOAc/H₂O and the layers separated; the organic phase was washed with brine, dried over Na₂SO₄ and concentrated in vacuo to afford the crude product which was purified by flash column chromatography on silica gel (heptanes/EtOAc=100:0 to 80:20) to afford the title compound. 1H NMR (400 MHz, CHLOROFORM-d) δ ppm 7.12-7.25 (m, 3H) 6.85-7.01 (m, 3H) 6.01 (s, 2H) 5.01 (s, 2H) 3.59 (s, 2H) 1.42 (s, 9H).

### Example 253-D

### (R)-tert-butyl 2-(2-((6-(3-(1-((tert-butoxycarbonyl)amino)ethyl)-2-fluorophenyl)benzo[d][1,3]dioxol-4-yl)methoxy)phenyl)acetate

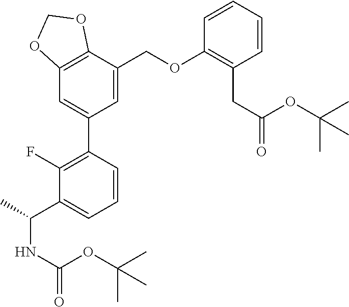 [SMILES: C[C@@H](NC(=O)OC(C)(C)C)c1cccc(-c2cc(COc3ccccc3CC(=O)OC(C)(C)C)c3c(c2)OCO3)c1F]

To a solution of tert-Butyl 2-(2-((6-bromobenzo[d][1,3]dioxol-4-yl)methoxy)phenyl)acetate (117 mg, 0.278 mmol) and (R)-tert-butyl (1-(2-fluoro-3-(4,4,5,5-tetramethyl-1,3,2-dioxaborolan-2-yl)phenyl)ethyl)carbamate (Intermediate 27-B) (112 mg, 0.305 mmol) in DMF (2.0 mL) was added a 2.0M solution of K₃PO₄ (0.555 mL, 1.111 mmol); this mixture was degassed for 10 minutes with N₂(g), and then PdCl₂(dppf).CH₂Cl₂ adduct (11.33 mg, 0.014 mmol) added. The reaction was sealed and heated at 110 C in an oil bath for 1 hr. The reaction was cooled to room temperature, filtered over a plug of Celite and the filtrate liquor was partitioned between EtOAc/H2O and the layers separated; the organic phase was brine washed, dried over Na2SO4, and concentrated in vacuo. The residue was purified by flash column chromatography on silica gel (heptanes/EtOAc=100:0 to 40:60) to afford the title compound. MS (ESI+) m/z 602.4 (M+Na).

### Example 253-E

### (R)-2-(2-((6-(3-(1-Aminoethyl)-2-fluorophenyl)benzo[d][1,3]dioxol-4-yl)methoxy)phenyl)acetic acid

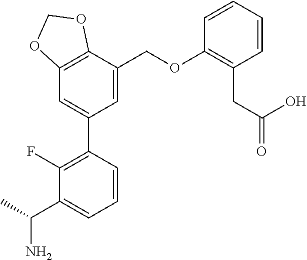 [SMILES: C[C@@H](N)c1cccc(-c2cc(COc3ccccc3CC(=O)O)c3c(c2)OCO3)c1F]

To a solution of (R)-tert-butyl 2-(2-((6-(3-(1-((tert-butoxycarbonyl)amino)ethyl)-2-fluorophenyl)benzo[d][1,3]dioxol-4-yl)methoxy)phenyl)acetate (100 mg, 0.173 mmol) in dichloromethane (1.0 mL) was added 4.0M HCl in dioxane (5.0 mL). This mixture was stirred for several hours and then concentrated in vacuo. The resulting residue was purified by HPLC (method B) to afford the title compound. ¹H NMR (400 MHz, DMSO-d₆) δ ppm 7.50 (t, J=6.63 Hz, 1H) 7.28-7.38 (m, 1H) 7.13-7.25 (m, 4H) 7.01-7.11 (m, 2H) 6.80-6.94 (m, 1H) 6.13 (s, 2H) 5.12 (s, 2H) 4.33 (q, J=6.99 Hz, 1H) 3.50 (s., 2H) 1.33 (d, J=6.57 Hz, 3H). HRMS calcd. for C₂₄H₂₂FNO₅ (M+H)+ 424.1560. found 424.1550.

### Example 254

### (R)-2-(2-((6-(3-(1-aminoethyl)phenyl)benzo[d][1,3]dioxol-4-yl)methoxy)phenyl)acetic acid

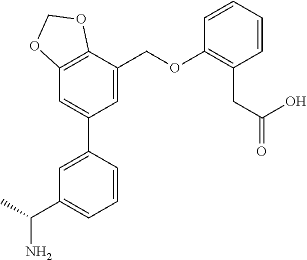 [SMILES: C[C@@H](N)c1cccc(-c2cc(COc3ccccc3CC(=O)O)c3c(c2)OCO3)c1]

The title compound was synthesized in a similar manner as Example 253. ¹H NMR (400 MHz, METHANOL-d4) δ ppm 8.12 (s, 1H) 7.52-7.64 (m, 2H) 7.40 (t, J=7.71 Hz, 1H) 7.26 (d, J=7.71 Hz, 1H) 7.14-7.21 (m, 2H) 7.09 (d, J=1.64 Hz, 1H) 6.94 (d, J=7.96 Hz, 1H) 6.88 (t, J=7.39 Hz, 1H) 6.06 (s, 2H) 5.08-5.31 (m, 2H) 4.45 (d, J=6.95 Hz, 1H) 3.50-3.70 (m, 2H) 1.66 (d, J=6.82 Hz, 3H) HRMS calcd. for C₂₄H₂₃NO₅ (M+H)⁺ 406.1654. found 406.1647.

### Example 255

### (S)-2-(2-((6-(3-(1-amino-2-hydroxyethyl)phenyl)benzo[d][1,3]dioxol-4-yl)methoxy)phenyl)acetic acid

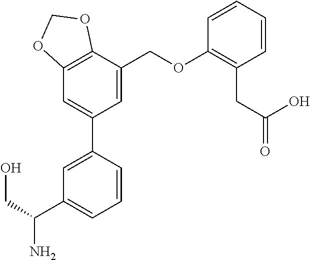 [SMILES: N[C@H](CO)c1cccc(-c2cc(COc3ccccc3CC(=O)O)c3c(c2)OCO3)c1]

The title compound was synthesized in a similar manner as Example 253. ¹H NMR (400 MHz, METHANOL-d₄) δ ppm 8.09 (s, 1H) 7.54-7.62 (m, 2H) 7.41 (t, J=7.71 Hz, 1H) 7.26 (d, J=7.70 Hz, 1H) 7.13-7.21 (m, 2H) 7.09 (d, J=1.77 Hz, 1H) 6.94 (d, J=7.83 Hz, 1H) 6.88 (td, J=7.39, 1.01 Hz, 1H) 6.06 (s, 2H) 5.10-5.24 (m, 2H) 4.32 (dd, J=8.97, 4.55 Hz, 1H) 3.79-4.01 (m, 2H) 3.50-3.68 (m, 2H). HRMS calcd. for C₂₄H₂₃NO₆ (M+H)⁺ , 422.1604. found 422.1589.

### Example 256

### Example 256-A

### (R)-tert-Butyl 2-(2-((3′-(1-((tert-butoxycarbonyl)amino)ethyl)-5-ethoxy-2′-fluoro-[1,1′-biphenyl]-3-yl)methoxy)phenyl)acetate

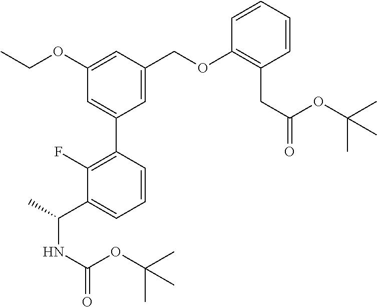 [SMILES: CCOc1cc(COc2ccccc2CC(=O)OC(C)(C)C)cc(-c2cccc([C@@H](C)NC(=O)OC(C)(C)C)c2F)c1]

To a solution of tert-Butyl 2-(2-((3-bromo-5-ethoxybenzyl)oxy)phenyl)acetate (Intermediate 57-D) (108 mg, 0.256 mmol) and (R)-tert-butyl (1-(2-fluoro-3-(4,4,5,5-tetramethyl-1,3,2-dioxaborolan-2-yl)phenyl)ethyl)carbamate (Intermediate 27-B) (103 mg, 0.282 mmol) in DMF (2.0 mL) was added a 2.0M solution of K₃PO₄ (0.513 mL, 1.025 mmol); this mixture was degassed for 10 minutes with N₂(g), and then PdCl₂(dppf).CH₂Cl₂ adduct (10.47 mg, 0.013 mmol) added. The reaction was sealed and heated at 110° C. in an oil bath for 1 hr. The reaction was cooled to room temperature, filtered over a plug of Celite and the filtrate liquor was partitioned between EtOAc/H₂O and the layers separated; the organic phase was brine washed, dried over Na₂SO₄, and concentrated in vacuo. The residue was purified by flash column chromatography on silica gel (heptanes/EtOAc=100:0 to 60:40) to afford the title compound. MS (ESI+) m/z 602.4 (M+Na).

### Example 256-B

### (R)-2-(2-((3′-(1-Aminoethyl)-5-ethoxy-2′-fluoro-[1,1′-biphenyl]-3-yl)methoxy)phenyl)acetic acid

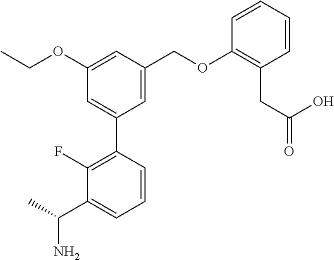 [SMILES: CCOc1cc(COc2ccccc2CC(=O)O)cc(-c2cccc([C@@H](C)N)c2F)c1]

To a solution of (R)-tert-butyl 2-(2-((3′-(1-((tert-butoxycarbonyl)amino)ethyl)-5-ethoxy-2′-fluoro-[1,1′-biphenyl]-3-yl)methoxy)phenyl)acetate (100 mg, 0.121 mmol) in dichloromethane (1.0 mL) was added 4.0M HCl in dioxane (5.0 mL). This mixture was stirred for several hours and then concentrated in vacuo. The resulting residue was purified by HPLC (method B) to afford the title compound. ¹H NMR (400 MHz, DMSO-d₆ TFA salt) δ ppm 12.17 (br s., 1H) 8.33 (br. s., 2H) 7.52-7.65 (m, 2H) 7.35-7.45 (m, 1H) 7.15-7.27 (m, 3H) 7.09 (s, 1H) 6.98-7.06 (m, 2H) 6.83-6.95 (m, 1H) 5.16 (s, 2H) 4.71 (br. s., 1H) 4.09 (q, J=6.95 Hz, 2H) 3.59 (s, 2H) 1.56 (d, J=6.82 Hz, 3H) 1.35 (t, J=7.01 Hz, 3H). HRMS calcd. for C₂₅H₂₆FNO₄ (M+H)⁺ 424.1924. found 424.1924.

### Example 257

### (R)-2-(2-((3′-(1-aminoethyl)-5-ethoxy-[1,1′-biphenyl]-3-yl)methoxy)phenyl)acetic acid

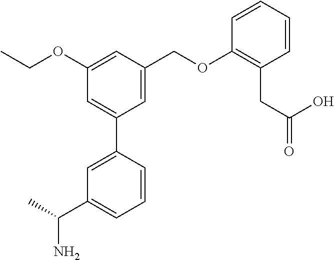 [SMILES: CCOc1cc(COc2ccccc2CC(=O)O)cc(-c2cccc([C@@H](C)N)c2)c1]

The title compound was synthesized in a similar manner as Example 256. ¹H NMR (400 MHz, METHANOL-d₄) δ ppm 8.13 (br. s., 1H) 7.60-7.69 (m, 2H) 7.44 (t, J=7.77 Hz, 1H) 7.31 (br. s., 1H) 7.14-7.22 (m, 2H) 7.11 (s, 1H) 6.91-6.99 (m, 2H) 6.87 (t, J=7.26 Hz, 1H) 5.02-5.33 (m, 2H) 4.44 (br. s., 1H) 4.13 (q, J=6.99 Hz, 2H) 3.50-3.68 (m, 2H) 1.65 (d, J=5.81 Hz, 3H) 1.42 (t, J=7.01 Hz, 3H). HRMS calcd. for C₂₅H₂₇NO₄ (M+H)⁺ 406.2018. found 406.2010.

### Example 258

### Example 258-A

### tert-Butyl 2-(2-((3-ethoxy-5-(4,4,5,5-tetramethyl-1,3,2-dioxaborolan-2-yl)benzyl)oxy)phenyl)acetate

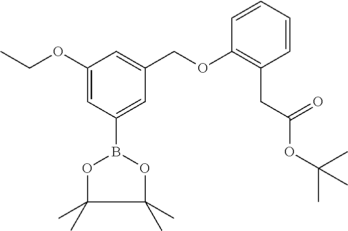 [SMILES: CCOc1cc(COc2ccccc2CC(=O)OC(C)(C)C)cc(B2OC(C)(C)C(C)(C)O2)c1]

To a solution of tert-Butyl 2-(2-((3-bromo-5-ethoxybenzyl)oxy)phenyl)acetate (242 mg, 0.574 mmol) (Intermediate 57-D) and 4,4,4′,4′,5,5,5′,5′-octamethyl-2,2′-bi(1,3,2-dioxaborolane) (219 mg, 0.862 mmol) in DMF (3.0 mL) was added KOAc (169 mg, 1.72 mmol); this mixture was degassed for 10 minutes with N₂(g), and then [1,1′-Bis(diphenylphosphino)ferrocene]dichloropalladium(II) complex with dichloromethane (23.5 mg, 0.029 mmol) added. The reaction was sealed and heated at 110° C. in an oil bath for 2 hr. The reaction was cooled to room temperature, filtered over a plug of Celite and the filtrate liquor was partitioned between EtOAc/H₂O and the layers separated; the organic phase was brine washed, dried over Na₂SO₄, and concentrated in vacuo. The residue was purified by flash column chromatography on silica gel (heptanes/EtOAc=100:0 to 70:30) to afford the title compound. 1H NMR (400 MHz, METHANOL-d4) δ ppm 7.37 (d, J=0.63 Hz, 1H) 7.11-7.28 (m, 4H) 6.99 (d, J=7.58 Hz, 1H) 6.90 (td, J=7.39, 1.01 Hz, 1H) 5.05 (s, 2H) 4.06 (q, J=6.99 Hz, 2H) 3.57 (s, 2H) 1.25-1.44 (m, 24H).

### Example 258-B

### tert-butyl 2-(2-((3′-(1-((tert-butoxycarbonyl)amino)-2-fluoroethyl)-5-ethoxy-2′-fluoro-[1,1′-biphenyl]-3-yl)methoxy)phenyl)acetate

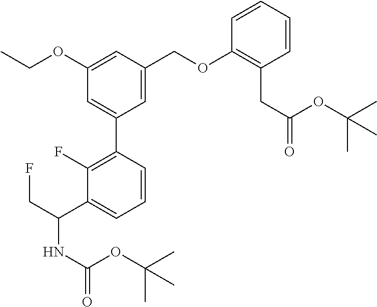 [SMILES: CCOc1cc(COc2ccccc2CC(=O)OC(C)(C)C)cc(-c2cccc(C(CF)NC(=O)OC(C)(C)C)c2F)c1]

To a solution of tert-butyl 2-(2-((3-ethoxy-5-(4,4,5,5-tetramethyl-1,3,2-dioxaborolan-2-yl)benzyl)oxy)phenyl)acetate (100 mg, 0.213 mmol) and tert-butyl (1-(3-bromo-2-fluorophenyl)-2-fluoroethyl)carbamate (Intermediate 33-C) (93 mg, 0.278 mmol) in DMF (1.0 mL) was added a 2.0M solution of K₃PO₄ (0.320 mL, 0.640 mmol); this mixture was degassed for 10 minutes with N₂(g), and then PdCl₂(dppf).CH₂Cl₂ adduct (8.71 mg, 0.011 mmol) added. The reaction was sealed and heated at 110° C. in an oil bath for 1 hr. The reaction was cooled to room temperature, filtered over a plug of Celite and the filtrate liquor was partitioned between EtOAc/H₂O and the layers separated; the organic phase was brine washed, dried over Na₂SO₄, and concentrated in vacuo. The residue was purified by flash column chromatography on silica gel (heptanes/EtOAc=100:0 to 70:30) to afford the title compound. MS (ESI+) m/z 620.4 (M+Na).

### Example 258-C

### 2-(2-((3′-(1-amino-2-fluoroethyl)-5-ethoxy-2′-fluoro-[1,1′-biphenyl]-3-yl)methoxy)phenyl)acetic acid

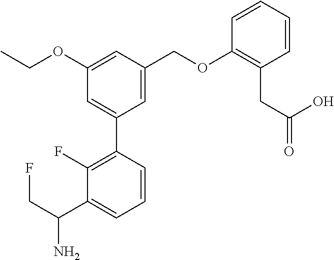 [SMILES: CCOc1cc(COc2ccccc2CC(=O)O)cc(-c2cccc(C(N)CF)c2F)c1]

To a solution of tert-butyl 2-(2-((3′-(1-((tert-butoxycarbonyl)amino)-2-fluoroethyl)-5-ethoxy-2′-fluoro-[1,1′-biphenyl]-3-yl)methoxy)phenyl)acetate (50 mg, 0.084 mmol) in dichloromethane (1.0 mL) was added 4.0M HCl in dioxane (5.0 mL). This mixture was stirred for several hours and then concentrated in vacuo. The resulting residue was purified by HPLC (method B) to afford the title compound. ¹H NMR (400 MHz, METHANOL-d₄) δ ppm 7.43-7.55 (m, 2H) 7.24-7.32 (m, 2H) 7.15-7.23 (m, 2H) 7.05 (s, 1H) 6.95-7.01 (m, 2H) 6.85-6.92 (m, 1H) 5.14 (s, 2H) 4.68-4.79 (m, 2H) 4.56-4.67 (m, 1H) 4.10 (q, J=6.99 Hz, 2H) 3.62 (s, 2H) 1.40 (t, J=7.01 Hz, 3H). HRMS calcd. for C₂₅H₂₅F₂NO₄ (M+H)⁺ 442.1830. found 442.1822.

### Example 259

### Example 259-A

### Methyl 3-bromo-5-((1-methylcyclopropyl)methoxy)benzoate

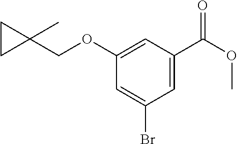 [SMILES: COC(=O)c1cc(Br)cc(OCC2(C)CC2)c1]

The title compound was synthesized as described in Intermediate 55 starting with methyl 3-bromo-5-hydroxylbenzoate (Intermediate 57-B) and (1-methylcyclopropyl)methanol (CAS #2746-14-7). ¹H NMR (400 MHz, CHLOROFORM-d) δ ppm 7.62 (t, J=1.52 Hz, 1H) 7.35 (dd, J=2.40, 1.26 Hz, 1H) 7.13 (t, J=2.08 Hz, 1H) 3.79 (s, 3H) 3.63 (s, 2H) 1.10 (s, 3H) 0.42 (m, 2H) 0.33 (m, 2H).

### Example 259-B

### (3-bromo-5-((1-methylcyclopropyl)methoxy)phenyl)methanol

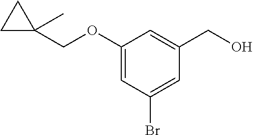

To a solution of methyl 3-bromo-5-((1-methylcyclopropyl)methoxy)benzoate (0.850 g, 2.84 mmol) in THF (18.0 mL) under nitrogen was added 1.0M lithium aluminum hydride in THF solution (3.13 mL, 3.13 mmol) and the reaction was stirred at 0° C. for 60 min and then quenched with slow dropwise addition of H₂O (1.0 mL), 15% NaOH soln (1.0 mL) and H₂O (3.0 mL); a white precipitate crashed out of the reaction; this mixture was stirred for 1.0 hr additional at room temperature and then the solid was filtered off. The remaining solution was diluted with EtOAc/H₂O and the layers partitioned; the organic phase was brine washed, dried over Na₂SO₄ and concentrated in vacuo. The residue was purified by flash column chromatography on silica gel (heptanes/EtOAc=100:0 to 60:40) to afford the title compound. 1H NMR (400 MHz, CHLOROFORM-d) δ ppm 7.00 (s, 1H) 6.89 (s, 1H) 6.77 (s, 1H) 4.56 (s, 2H) 3.64 (s, 2H) 1.62 (br. s, 1H) 1.14 (s, 3H) 0.42-0.52 (m, 2H) 0.33-0.38 (m, 2H).

### Example 259-C

### tert-butyl 2-(2-((3-bromo-5-((1-methylcyclopropyl)methoxy)benzyl)oxy)phenyl)acetate

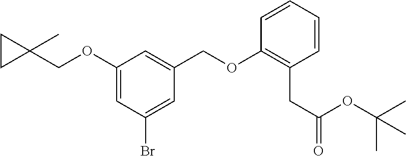 [SMILES: CC1(COc2cc(Br)cc(COc3ccccc3CC(=O)OC(C)(C)C)c2)CC1]

The title compound was synthesized as described in Intermediate 55 starting with (3-bromo-5-((1-methylcyclopropyl)methoxy)phenyl)methanol and tert-Butyl 2-(2-hydroxyphenyl)acetate (Intermediate 21). ¹H NMR (400 MHz, CHLOROFORM-d) δ ppm 7.11-7.24 (m, 3H) 6.81-7.04 (m, 4H) 5.01 (s, 2H) 3.72 (s, 2H) 3.60 (s, 2H) 1.38-1.43 (m, 9H) 1.21 (s, 3H) 0.49-0.57 (m, 2H) 0.37-0.45 (m, 2H).

### Example 259-D

### (R)-tert-butyl 2-(2-((3′-(1-((tert-butoxycarbonyl)amino)ethyl)-2′-fluoro-5-((1-methylcyclopropyl)methoxy)-[1,1′-biphenyl]-3-yl)methoxy)phenyl)acetate

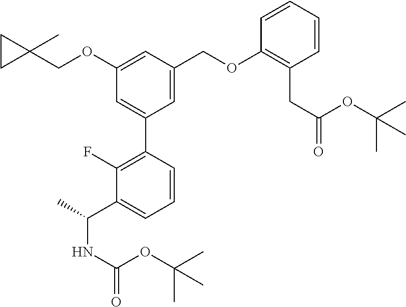 [SMILES: C[C@@H](NC(=O)OC(C)(C)C)c1cccc(-c2cc(COc3ccccc3CC(=O)OC(C)(C)C)cc(OCC3(C)CC3)c2)c1F]

To a solution of tert-butyl 2-(2-((3-bromo-5-((1-methylcyclopropyl)methoxy)benzyl)oxy)phenyl)acetate (115 mg, 0.249 mmol) and (R)-tert-butyl (1-(2-fluoro-3-(4,4,5,5-tetramethyl-1,3,2-dioxaborolan-2-yl)phenyl)ethyl)carbamate (Intermediate 27-B) (100 mg, 0.274 mmol) in DMF (2.0 mL) was added a 2.0M solution of K₃PO₄ (0.498 mL, 0.997 mmol); this mixture was degassed for 10 minutes with N₂(g), and then PdCl₂(dppf).CH₂Cl₂ adduct (10.17 mg, 0.012 mmol) added. The reaction was sealed and heated at 110 C in an oil bath for 1 hr. The reaction was cooled to room temperature, filtered over a plug of Celite and the filtrate liquor was partitioned between EtOAc/H2O and the layers separated; the organic phase was brine washed, dried over Na₂SO₄, and concentrated in vacuo. The residue was purified by flash column chromatography on silica gel (heptanes/EtOAc=100:0 to 60:40) to afford the title compound. MS (ESI+) m/z 642.6 (M+Na).

### Example 259-E

### (R)-2-(2-((3′-(1-aminoethyl)-2′-fluoro-5-((1-methylcyclopropyl)methoxy)-[1,1′-biphenyl]-3-yl)methoxy)phenyl)acetic acid

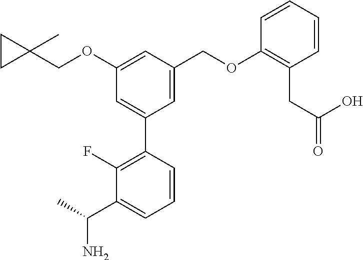 [SMILES: C[C@@H](N)c1cccc(-c2cc(COc3ccccc3CC(=O)O)cc(OCC3(C)CC3)c2)c1F]

To a solution of (R)-tert-butyl 2-(2-((3′-(1-((tert-butoxycarbonyl)amino)ethyl)-2′-fluoro-5-((1-methylcyclopropyl)methoxy)-[1,1′-biphenyl]-3-yl)methoxy)phenyl)acetate (80 mg, 0.129 mmol) in dichloromethane (1.0 mL) was added 4.0M HCl in dioxane (5.0 mL). This mixture was stirred for several hours and then concentrated in vacuo. The resulting residue was purified by HPLC (method B) to afford the title compound. ¹H NMR (400 MHz, METHANOL-d4) δ ppm 7.49-7.56 (m, 1H) 7.39-7.48 (m, 2H) 7.27-7.34 (m, 1H) 7.17-7.22 (m, 1H) 7.11-7.17 (m, 1H) 7.00 (s, 1H) 6.91-6.98 (m, 2H) 6.86 (t, J=6.95 Hz, 1H) 5.14 (s, 2H) 4.68-4.76 (m, 1H) 3.83 (s, 2H) 3.57 (s, 2H) 1.67 (d, J=6.82 Hz, 3H) 1.25 (s, 3H) 0.52-0.65 (m, 2H) 0.36-0.45 (m, 2H). HRMS calcd. for C₂₈H₃₀FNO₄ (M+H)⁺ 464.2237. found 464.2209.

### Example 260

### (S)-2-(2-((3′-(1-aminoethyl)-2′-fluoro-5-((1-methylcyclopropyl)methoxy)-[1,1′-biphenyl]-3-yl)methoxy)phenyl)acetic acid

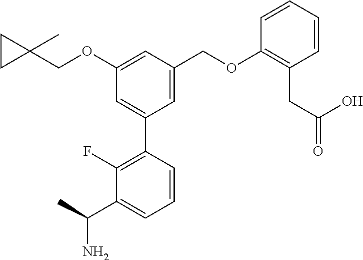 [SMILES: C[C@H](N)c1cccc(-c2cc(COc3ccccc3CC(=O)O)cc(OCC3(C)CC3)c2)c1F]

The title compound was synthesized in the same manner as Example 259. ¹H NMR (400 MHz, METHANOL-d4) δ ppm 7.48-7.54 (m, 1H) 7.43 (d, J=13.39 Hz, 2H) 7.27-7.33 (m, 1H) 7.19 (d, J=9.09 Hz, 1H) 7.14 (t, J=7.71 Hz, 1H) 7.00 (s, 1H) 6.91-6.98 (m, 2H) 6.86 (t, J=7.58 Hz, 1H) 5.14 (s, 2H) 4.67-4.74 (m, 1H) 3.83 (s, 2H) 3.57 (s, 2H) 1.66 (d, J=6.82 Hz, 3H) 1.25 (s, 3H) 0.54-0.62 (m, 2H) 0.38-0.44 (m, 2H) HRMS calcd. for C₂₈H₃₀FNO₄ (M+H) 464.2237. found 464.2207.

### Example 261

### (R)-2-(2-((3′-(1-aminoethyl)-5-((1-methylcyclopropyl)methoxy)-[1,1′-biphenyl]-3-yl)methoxy)phenyl)acetic acid

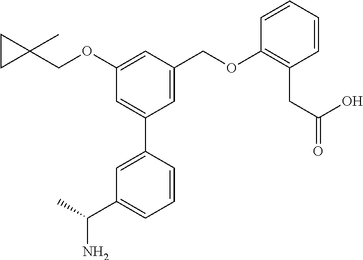 [SMILES: C[C@@H](N)c1cccc(-c2cc(COc3ccccc3CC(=O)O)cc(OCC3(C)CC3)c2)c1]

The title compound was synthesized in the same manner as Example 259. ¹H NMR (400 MHz, METHANOL-d4) δ ppm 8.17 (s, 1H) 7.61-7.70 (m, 2H) 7.44 (t, J=7.71 Hz, 1H) 7.31 (d, J=7.58 Hz, 1H) 7.14-7.21 (m, 2H) 7.11 (t, J=1.89 Hz, 1H) 6.90-6.97 (m, 2H) 6.87 (td, J=7.39, 1.01 Hz, 1H) 5.07-5.22 (m, 2H) 4.46 (q, J=6.95 Hz, 1H) 3.81-3.88 (m, 2H) 3.50-3.69 (m, 2H) 1.67 (d, J=6.82 Hz, 3H) 1.26 (s, 3H) 0.54-0.63 (m, 2H) 0.40-0.46 (m, 2H) HRMS calcd. for C₂₈H₃₁NO₄ (M+H)⁺ 446.2314. found 446.2331.

### Example 262

### (R)-2-(2-((3′-(1-aminoethyl)-2′-fluoro-5-((3-methyloxetan-3-yl)methoxy)-[1,1′-biphenyl]-3-yl)methoxy)phenyl)acetic acid

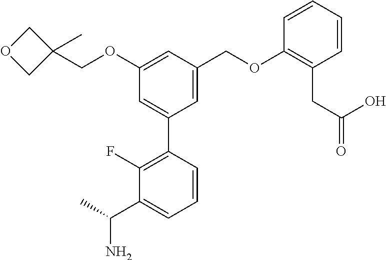 [SMILES: C[C@@H](N)c1cccc(-c2cc(COc3ccccc3CC(=O)O)cc(OCC3(C)COC3)c2)c1F]

The title compound was synthesized in the same manner as Example 259. ¹H NMR (400 MHz, METHANOL-d4) δ ppm 7.50-7.56 (m, 1H) 7.42-7.48 (m, 2H) 7.28-7.34 (m, 1H) 7.20 (d, J=7.45 Hz, 1H) 7.09-7.17 (m, 2H) 7.05 (s, 1H) 6.94 (d, J=7.45 Hz, 1H) 6.83-6.90 (m, 1H) 5.16 (s, 2H) 4.70 (d, J=5.94 Hz, 2H) 4.49-4.79 (m, 1H) 4.46 (d, J=5.94 Hz, 2H) 4.13 (s, 2H) 3.58 (s, 2H) 1.65 (d, J=6.82 Hz, 3H) 1.46 (s, 3H). HRMS calcd. for C₂₈H₃₀FNO₅ (M+H)⁺ 480.2186. found 480.2150.

### Example 263

### (R)-2-(2-((3′-(1-aminoethyl)-5-((3-methyloxetan-3-yl)methoxy)-[1,1′-biphenyl]-3-yl)methoxy)phenyl)acetic acid

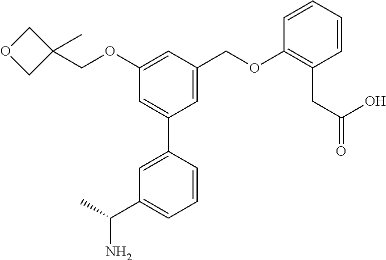 [SMILES: C[C@@H](N)c1cccc(-c2cc(COc3ccccc3CC(=O)O)cc(OCC3(C)COC3)c2)c1]

The title compound was synthesized in the same manner as Example 259. ¹H NMR (400 MHz, METHANOL-d4) δ ppm 7.99 (s, 1H) 7.56-7.66 (m, 2H) 7.43 (t, J=7.64 Hz, 1H) 7.32 (d, J=7.71 Hz, 1H) 7.12-7.22 (m, 3H) 7.06 (s, 1H) 6.96 (d, J=7.58 Hz, 1H) 6.83-6.91 (m, 1H) 5.06-5.28 (m, 2H) 4.71 (d, J=5.94 Hz, 2H) 4.46 (d, J=5.94 Hz, 2H) 4.33 (q, J=6.11 Hz, 1H) 4.15 (s, 2H) 3.51-3.69 (m, 2H) 1.58 (d, J=6.82 Hz, 3H) 1.47 (s, 3H). HRMS calcd. for C₂₈H₃₁NO₅(M+H)⁺ 462.2280. found 462.2252.

### Example 264

### 2-(2-((3′-((R)-1-aminoethyl)-2′-fluoro-5-((tetrahydro-2H-pyran-2-yl)methoxy)-[1,1′-biphenyl]-3-yl)methoxy)phenyl)acetic acid

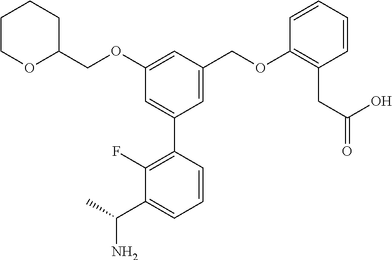 [SMILES: C[C@@H](N)c1cccc(-c2cc(COc3ccccc3CC(=O)O)cc(OCC3CCCCO3)c2)c1F]

The title compound was synthesized in the same manner as Example 259. ¹H NMR (600 MHz, METHANOL-d4) δ ppm 7.54 (t, J=7.15 Hz, 1H) 7.40-7.48 (m, 2H) 7.28-7.35 (m, 1H) 7.19 (d, J=6.69 Hz, 1H) 7.15 (t, J=7.34 Hz, 1H) 6.98-7.08 (m, 2H) 6.94 (d, J=8.07 Hz, 1H) 6.87 (t, J=7.43 Hz, 1H) 5.16 (s, 2H) 3.95-4.05 (m, 3H) 3.70-3.80 (m, 1H) 3.59 (s, 2H) 3.48-3.56 (m, 1H) 3.09-3.22 (m, 1H) 1.91 (br. s., 1H) 1.73 (d, J=12.10 Hz, 1H) 1.68 (d, J=6.88 Hz, 3H) 1.55-1.65 (m, 2H) 1.46-1.53 (m, 1H) 1.29 (t, J=7.24 Hz, 1H). HRMS calcd. for C₂₉H₃₂FNO₅(M+H)⁺ 494.2298. found 494.2315.

### Example 265

### 2-(2-((3′-((R)-1-aminoethyl)-5-((tetrahydro-2H-pyran-2-yl)methoxy)-[1,1′-biphenyl]-3-yl)methoxy)phenyl)acetic acid

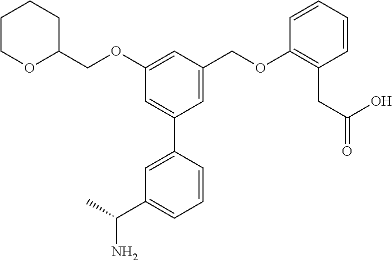 [SMILES: C[C@@H](N)c1cccc(-c2cc(COc3ccccc3CC(=O)O)cc(OCC3CCCCO3)c2)c1]

The title compound was synthesized in the same manner as Example 259. ¹H NMR (400 MHz, METHANOL-d₄) δ ppm 8.18 (s, 1H) 7.70 (s, 1H) 7.65 (d, J=7.96 Hz, 1H) 7.44 (t, J=7.64 Hz, 1H) 7.31 (d, J=7.58 Hz, 1H) 7.11-7.23 (m, 3H) 6.92-6.99 (m, 2H) 6.87 (td, J=7.39, 1.01 Hz, 1 H) 5.08-5.24 (m, 2H) 4.46 (q, J=6.91 Hz, 1H) 3.96-4.08 (m, 3H) 3.71-3.81 (m, 1H) 3.50-3.68 (m, 3H) 1.88-1.96 (m, 1H) 1.75 (d, J=12.13 Hz, 1H) 1.66 (d, J=6.95 Hz, 3H) 1.45-1.64 (m, 4H). HRMS calcd. for C₂₉H₃₃NO₅ (M+H)⁺ 476.2392. found 476.2404.

### Example 266

### 2-(2-((3′-((R)-1-aminoethyl)-2′-fluoro-5-(pyrrolidin-2-ylmethoxy)-[1,1′-biphenyl]-3-yl)methoxy)phenyl)acetic acid

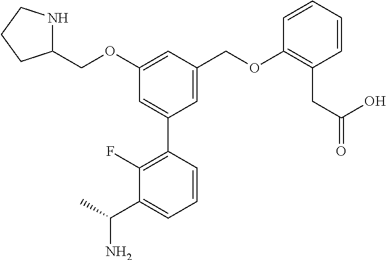 [SMILES: C[C@@H](N)c1cccc(-c2cc(COc3ccccc3CC(=O)O)cc(OCC3CCCN3)c2)c1F]

The title compound was synthesized in the same manner as Example 259. ¹H NMR (600 MHz, METHANOL-d₄) δ ppm 7.47 (t, J=7.06 Hz, 1H) 7.35-7.40 (m, 2H) 7.25 (t, J=7.70 Hz, 1H) 7.17-7.22 (m, 2H) 7.13-7.17 (m, 1H) 7.05 (s, 1H) 6.95 (d, J=8.25 Hz, 1H) 6.87 (t, J=7.38 Hz, 1H) 5.18 (d, J=2.11 Hz, 2H) 4.45 (q, J=6.66 Hz, 1H) 4.31 (d, J=6.79 Hz, 2H) 3.80-3.90 (m, 1H) 3.56 (d, J=3.48 Hz, 2H) 3.24 (d, J=1.83 Hz, 2H) 2.13-2.22 (m, 1H) 2.05-2.13 (m, 1H) 1.96-2.05 (m, 1H) 1.82-1.91 (m, 1H) 1.48 (d, J=6.69 Hz, 3H). HRMS calcd. for C₂₈H₃₁FN₂O₄(M+H)⁺ 479.2546. found 479.2556.

### Example 267

### 2-(2-((3′-((R)-1-aminoethyl)-5-(pyrrolidin-2-ylmethoxy)-[1,1′-biphenyl]-3-yl)methoxy)phenyl)acetic acid

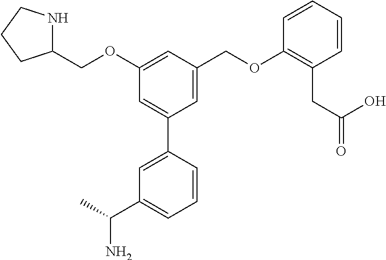 [SMILES: C[C@@H](N)c1cccc(-c2cc(COc3ccccc3CC(=O)O)cc(OCC3CCCN3)c2)c1]

The title compound was synthesized in the same manner as Example 259. ¹H NMR (400 MHz, METHANOL-d₄) δ ppm 8.01 (s, 1H) 7.55-7.66 (m, 2H) 7.44 (t, J=7.71 Hz, 1H) 7.33 (d, J=7.58 Hz, 1H) 7.07-7.21 (m, 4H) 6.95 (d, J=8.21 Hz, 1H) 6.87 (t, J=7.39 Hz, 1H) 5.12-5.23 (m, 2H) 4.38 (q, J=6.86 Hz, 1H) 4.19-4.26 (m, 1H) 4.10-4.18 (m, 1H) 3.68-3.78 (m, 1H) 3.50-3.64 (m, 2H) 3.04-3.24 (m, 2H) 2.07-2.19 (m, 1H) 1.88-2.06 (m, 2H) 1.71-1.84 (m, 1H) 1.61 (d, J=6.82 Hz, 3H). HRMS calcd. for C₂₈H₃₂N₂O₄ (M+H)⁺ 461.2636. found 461.2657.

### Example 268

### Example 268-A

### Ethyl 2-(2-((3-bromobenzyl)oxy)phenyl)acetate

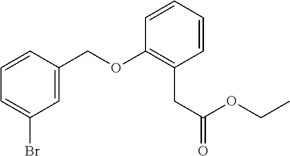 [SMILES: CCOC(=O)Cc1ccccc1OCc1cccc(Br)c1]

To a solution of EtOH (200 mL) was added AcCl (2.0 mL). The mixture was stirred for 5 min at rt. and then methyl 2-(2-((3-bromobenzyl)oxy)phenyl)acetate (Example 101-A) (1.0 g, 2.98 mmol) was added to this solution and it was refluxed at 80° C. overnight. Saturated bicarbonate solution (10.0 mL) was added and then the reaction was concentrated in vacuo to remove most of the EtOH. The residue was partitioned between EtOAc/saturated bicarbonate solution and extracted. The organic phase was washed with brine solution, dried over Na₂SO₄ and concentrated in vacuo to afford the crude product. The residue was purified by flash column chromatography on silica gel (heptanes/EtOAc=100:0 to 60:40) to afford the title compound. 1H NMR (400 MHz, Methanol-d4) δ 7.62 (t, J=0.9 Hz, 1H), 7.46 (ddd, J=8.0, 1.9, 1.0 Hz, 1H), 7.43-7.37 (m, 1H), 7.33-7.17 (m, 3H), 7.00 (dd, J=8.3, 1.1 Hz, 1H), 6.92 (td, J=7.4, 1.1 Hz, 1H), 5.07 (s, 2H), 4.08 (q, J=7.2 Hz, 2H), 3.65 (s, 2H), 1.17 (t, J=7.1 Hz, 3H).

### Example 268-B

### (S)-ethyl 2-(2-((3′-(1-((tert-butoxycarbonyl)amino)-2-hydroxyethyl)-[1,1′-biphenyl]-3-yl)methoxy)phenyl)acetate

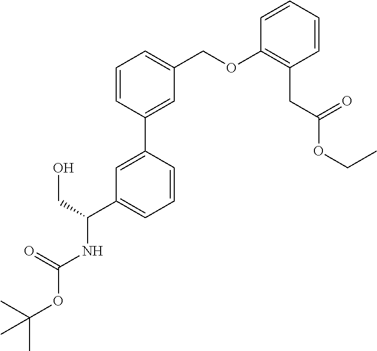 [SMILES: CCOC(=O)Cc1ccccc1OCc1cccc(-c2cccc([C@@H](CO)NC(=O)OC(C)(C)C)c2)c1]

To a solution of ethyl 2-(2-((3-bromobenzyl)oxy)phenyl)acetate (100 mg, 0.286 mmol) and (S)-tert-butyl (2-hydroxy-1-(3-(4,4,5,5-tetramethyl-1,3,2-dioxaborolan-2-yl)phenyl)ethyl)carbamate (Example 34-B) (114 mg, 0.315 mmol) in DMF (6.0 mL) was added a 2.0M solution of K₃PO₄ (0.572 mL, 1.14 mmol). This mixture was degassed for 10 minutes with N₂(g), and then [1,1′-Bis(diphenylphosphino)ferrocene]dichloropalladium(II) complex with dichloromethane (11.7 mg, 0.014 mmol) added. The reaction was sealed and heated at 110 C in an oil bath for 1 hr. The reaction was cooled to room temperature, filtered over a plug of Celite and the filtrate liquor was partitioned between EtOAc/H₂O and the layers separated. The organic phase was brine washed, dried over Na₂SO₄, and concentrated in vacuo. The residue was purified by flash column chromatography on silica gel (heptanes/EtOAc=100:0 to 30:70) to afford the title compound. MS (ESI+) m/z 406.3 (M−BOC+H).

### Example 268-C

### (S)-Ethyl 2-(2-((3′-(1-amino-2-hydroxyethyl)-[1,1′-biphenyl]-3-yl)methoxy)phenyl)acetate

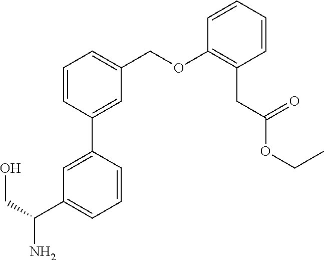 [SMILES: CCOC(=O)Cc1ccccc1OCc1cccc(-c2cccc([C@H](N)CO)c2)c1]

To a solution of (S)-ethyl 2-(2-((3′-(1-((tert-butoxycarbonyl)amino)-2-hydroxyethyl)-[1,1′-biphenyl]-3-yl)methoxy)phenyl)acetate (200 mg, 0.396 mmol) in dichloromethane (1.0 mL) was added 4.0M HCl in dioxane (5.0 mL). This mixture was stirred for several hours at r.t. and then concentrated in vacuo. The residue was purified by flash column chromatography on silica gel (DCM/MeOH=100:0 to 90:10) to afford the title compound. ¹H NMR (400 MHz, METHANOL-d₄) δ ppm 7.71 (d, J=15.16 Hz, 2H) 7.59 (dd, J=7.52, 1.83 Hz, 2H) 7.39-7.48 (m, 3H) 7.34-7.38 (m, 1H) 7.17-7.28 (m, 2H) 7.04 (d, J=7.96 Hz, 1H) 6.87-6.95 (m, 1H) 5.17 (s, 2H) 4.56 (br. s., 1H) 3.99 (q, J=7.07 Hz, 2H) 3.79 (dd, J=10.93, 4.74 Hz, 1H) 3.64-3.68 (m, 1H), 3.66 (s, 2H), 1.06 (t, J=7.14 Hz, 3H). HRMS calcd. for C₂₅H₂₇NO₄ (M+H)⁺ 406.2018. found 406.2004.

### Example 269

### Example 269-A

### Tert-butyl 2-(2-((3-allyl-5-chlorobenzyl)oxy)phenyl)acetate

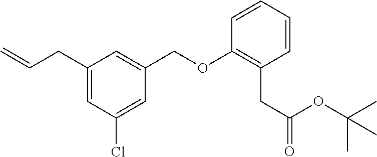 [SMILES: C=CCc1cc(Cl)cc(COc2ccccc2CC(=O)OC(C)(C)C)c1]

To a solution of tert-Butyl 2-(2-((3-bromo-5-chlorobenzyl)oxy)phenyl)acetate (Intermediate 53) (1.0 g, 2.43 mmol) in DMF (10.0 mL) was added allyltributylstannane (CAS #24850-33-7) (0.885 g, 2.67 mmol). This mixture was degassed for 10 minutes with N₂(g), and then tetrakis(triphenylphosphine)palladium(0) (CAS #14221-01-3) (140 mg, 0.121 mmol) added. This mixture was heated in a sealed vessel at 120° C. overnight. The reaction was filtered over a pad of Celite with 50 wt % KF, partitioned with EtOAc/H₂O and extracted. The organic phase was washed with brine, dried over Na₂SO₄ and concentrated in vacuo to afford a light yellow oil as crude product. The residue was purified by flash column chromatography on silica gel (heptanes/EtOAc=100:0 to 70:30) to afford the title compound. ¹H NMR (400 MHz, CHLOROFORM-d) δ ppm 7.30 (s, 1H) 7.18-7.25 (m, 2H) 7.10-7.14 (m, 2H) 6.94 (td, J=7.42, 1.07 Hz, 1H) 6.88 (d, J=8.08 Hz, 1H) 5.85-6.01 (m, 1H) 5.11-5.14 (m, 1H) 5.09 (dq, J=6.58, 1.55 Hz, 1H) 5.02 (s, 2H) 3.60 (s, 2H) 3.37 (d, J=6.82 Hz, 2H) 1.36-1.45 (m, 9H).

### Example 269-B

### Tert-butyl 2-(2-((3-chloro-5-(3-hydroxypropyl)benzyl)oxy)phenyl)acetate

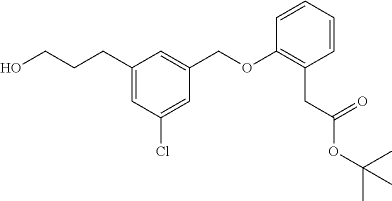 [SMILES: CC(C)(C)OC(=O)Cc1ccccc1OCc1cc(Cl)cc(CCCO)c1]

A solution of tert-butyl 2-(2-((3-allyl-5-chlorobenzyl)oxy)phenyl)acetate (870 mg, 2.33 mmol) in THF (26.0 mL) was cooled to 0° C. under nitrogen and 9-BBN (0.5M in THF, 14.0 mL, 7.0 mmol) was added dropwise. The reaction was warmed to room temperature and stirred overnight. The mixture was then cooled again to 0° C. and NaOH (2.0M aqueous, 7.0 mL, 14.0 mmol) and H₂O₂ (50% aqueous, 0.858 mL, 14.0 mmol) were added, and then warmed again to room temperature. After 15 minutes the reaction mixture was diluted with saturated aqueous sodium thiosulfate and EtOAc, the layers were separated and the aqueous layer was extracted again with EtOAc. The combined organic layers were dried over Na₂SO₄, and concentrated in vacuo. The residue was purified by flash column chromatography on silica gel (heptanes/EtOAc=100:0 to 40:60) to afford the title compound. 1H NMR (400 MHz, CHLOROFORM-d) δ ppm 7.18-7.25 (m, 3H) 7.14 (d, J=8.97 Hz, 2H) 6.91-6.97 (m, 1H) 6.85 (d, J=8.08 Hz, 1H) 5.04 (s, 2H) 3.63 (br. s, 1H) 3.59-3.62 (m, 4H) 2.71 (t, J=7.52 Hz, 2H) 1.77-1.95 (m, 2H) 1.35-1.46 (m, 9H).

### Example 269-C

### Tert-butyl 2-(2-((3-chloro-5-(3-fluoropropyl)benzyl)oxy)phenyl)acetate

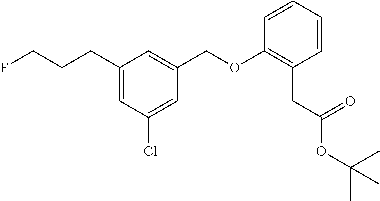 [SMILES: CC(C)(C)OC(=O)Cc1ccccc1OCc1cc(Cl)cc(CCCF)c1]

To a solution of tert-butyl 2-(2-((3-chloro-5-(3-hydroxypropyl)benzyl)oxy)phenyl)acetate (395 mg, 1.010 mmol) was added perfluoro-1-butanesulfonyl fluoride (CAS #375-72-4) (611 mg, 2.02 mmol), diisopropylethylamine trihydrofluoride (CAS #131600-43-6) (287 mg, 1.586 mmol) and diisopropylethylamine (588 mg, 4.55 mmol). This mixture was sealed and heated at 45° C. overnight. The reaction was partitioned EtOAc/H₂O and the layers separated. The organic layer was washed with saturated sodium bicarbonate solution, brine solution, dried over Na₂SO₄ and concentrated in vacuo. The residue was purified by flash column chromatography on silica gel (heptanes/EtOAc=100:0 to 80:20) to afford the title compound. ¹H NMR (400 MHz, CHLOROFORM-d) δ ppm 7.30 (s, 1H) 7.18-7.25 (m, 2H) 7.11-7.16 (m, 2H) 6.94 (td, J=7.45, 1.01 Hz, 1H) 6.87 (d, J=8.21 Hz, 1H) 5.03 (s, 2H) 4.51 (t, J=5.87 Hz, 1H) 4.39 (t, J=5.87 Hz, 1H) 3.60 (s, 2H) 2.65-2.81 (m, 2H) 1.88-2.13 (m, 2H) 1.40 (s, 9H).

### Example 269-D

### (R)-tert-butyl 2-(2-((3′-(1-((tert-butoxycarbonyl)amino)ethyl)-2′-fluoro-5-(3-fluoropropyl)-[1,1′-biphenyl]-3-yl)methoxy)phenyl)acetate

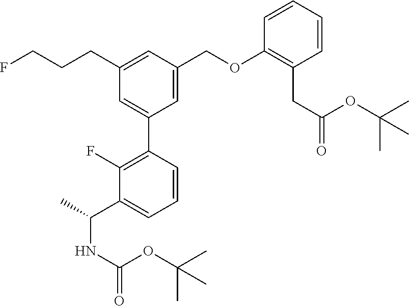 [SMILES: C[C@@H](NC(=O)OC(C)(C)C)c1cccc(-c2cc(CCCF)cc(COc3ccccc3CC(=O)OC(C)(C)C)c2)c1F]

To a solution of tert-butyl 2-(2-((3-chloro-5-(3-fluoropropyl)benzyl)oxy)phenyl)acetate (100 mg, 0.255 mmol) and (R)-tert-butyl (1-(2-fluoro-3-(4,4,5,5-tetramethyl-1,3,2-dioxaborolan-2-yl)phenyl)ethyl)carbamate (Intermediate 27-B) (139 mg, 0.382 mmol) in DMF (1.0 mL) was added a 2.0M solution of K₃PO₄ (0.636 mL, 1.272 mmol); this mixture was degassed for 10 minutes with N₂(g), and then S-Phos palladacycle (8.55 mg, 0.013 mmol) was added. The reaction was sealed and heated at 110° C. in an oil bath for 1.0 hr. The reaction was cooled to room temperature, filtered over a plug of Celite and the filtrate liquor was partitioned between EtOAc/H₂O and the layers separated; the organic phase was washed with brine, dried over Na₂SO₄, and concentrated in vacuo. The residue was purified by flash column chromatography on silica gel (heptanes/EtOAc=100:0 to 60:40) to afford the title compound. MS (ESI+) m/z 618.4 (M+Na).

### Example 269-E

### (R)-2-(2-((3′-(1-aminoethyl)-2′-fluoro-5-(3-fluoropropyl)-[1,1′-biphenyl]-3-yl)methoxy)phenyl)acetic acid

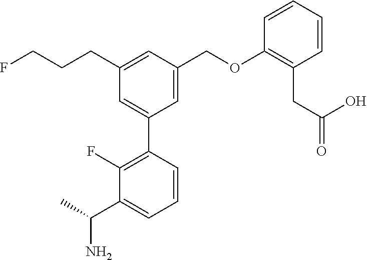 [SMILES: C[C@@H](N)c1cccc(-c2cc(CCCF)cc(COc3ccccc3CC(=O)O)c2)c1F]

To a solution of (R)-tert-butyl 2-(2-((3′-(1-((tert-butoxycarbonyl)amino)ethyl)-2′-fluoro-5-(3-fluoropropyl)-[1,1′-biphenyl]-3-yl)methoxy)phenyl)acetate (120 mg, 0.201 mmol) in dichloromethane (1.0 mL) was added 4.0M HCl in dioxane (5.0 mL). This mixture was stirred for several hours and then concentrated in vacuo. The resulting residue was purified by HPLC (method B) to afford the title compound. 1H NMR (400 MHz, METHANOL-d4) δ ppm 7.70 (s, 1H) 7.53 (td, J=7.58, 1.77 Hz, 1H) 7.41-7.48 (m, 1H) 7.27-7.34 (m, 3H) 7.10-7.22 (m, 2H) 6.95 (d, J=7.71 Hz, 1H) 6.86 (td, J=7.39, 0.88 Hz, 1H) 5.16 (s, 2H) 4.72 (q, J=6.91 Hz, 1H) 4.52 (t, J=6.00 Hz, 1H) 4.40 (t, J=5.94 Hz, 1H) 3.57 (s, 2H) 2.74-2.89 (m, 2H) 1.96-2.16 (m, 2H) 1.67 (d, J=6.95 Hz, 3H). HRMS calcd. for C₂₆H₂₇F₂NO₃ (M+H)⁺ 440.2037. found 440.2024.

### Example 270

### (S)-2-(2-((3′-(1-aminoethyl)-2′-fluoro-5-(3-fluoropropyl)-[1,1′-biphenyl]-3-yl)methoxy)phenyl)acetic acid

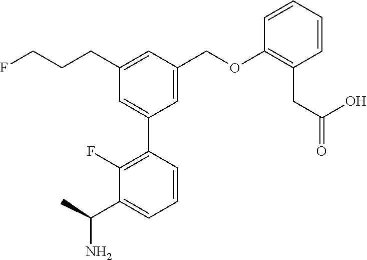 [SMILES: C[C@H](N)c1cccc(-c2cc(CCCF)cc(COc3ccccc3CC(=O)O)c2)c1F]

The title compound was synthesized as described in Example 269. ¹H NMR (400 MHz, METHANOL-d₄) δ ppm 7.65 (s, 1H) 7.42-7.53 (m, 2H) 7.27-7.34 (m, 3H) 7.20 (dd, J=7.45, 1.52 Hz, 1H) 7.11-7.17 (m, 1H) 6.95 (d, J=7.33 Hz, 1H) 6.86 (td, J=7.45, 1.01 Hz, 1H) 5.16 (s, 2H) 4.66 (q, J=6.78 Hz, 1H) 4.52 (t, J=5.94 Hz, 1H) 4.40 (t, J=5.94 Hz, 1H) 3.54-3.61 (m, 2H) 2.73-2.88 (m, 2H) 1.95-2.14 (m, 2H) 1.63 (d, J=6.95 Hz, 3H). HRMS calcd. for C₂₆H₂₇F₂NO₃ (M+H)⁺ 440.2037. found 440.1989.

### Example 271

### (R)-2-(2-((3′-(1-aminoethyl)-5-(3-fluoropropyl)-[1,1′-biphenyl]-3-yl)methoxy)phenyl)acetic acid

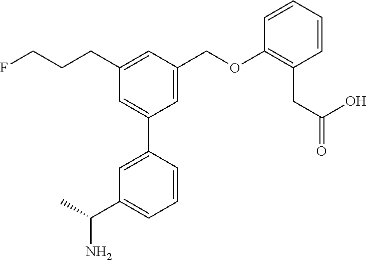 [SMILES: C[C@@H](N)c1cccc(-c2cc(CCCF)cc(COc3ccccc3CC(=O)O)c2)c1]

The title compound was synthesized as described in Example 269. H NMR (400 MHz, METHANOL-d₄) δ ppm 8.16 (s, 1H) 7.91 (s, 1H) 7.62-7.70 (m, 1H) 7.40-7.48 (m, 2H) 7.31 (d, J=7.71 Hz, 1H) 7.22 (s, 1H) 7.14-7.20 (m, 2H) 6.97 (d, J=7.83 Hz, 1H) 6.87 (td, J=7.39, 1.01 Hz, 1H) 5.06-5.30 (m, 2H) 4.52 (t, J=5.94 Hz, 1H) 4.38-4.48 (m, 2H) 3.47-3.71 (m, 2H) 2.75-2.91 (m, 2H) 1.95-2.17 (m, 2H) 1.65 (d, J=6.82 Hz, 3H). HRMS calcd. for C₂₆H₂₈FNO₃ (M+H)⁺ 422.1831. found 422.1802.

### Example 272

### (S)-2-(2-((3′-(1-amino-2-hydroxyethyl)-5-(3-fluoropropyl)-[1,1′-biphenyl]-3-yl)methoxy)phenyl)acetic acid

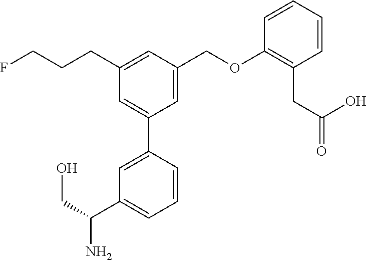 [SMILES: N[C@H](CO)c1cccc(-c2cc(CCCF)cc(COc3ccccc3CC(=O)O)c2)c1]

The title compound was synthesized as described in Example 269. ¹H NMR (400 MHz, METHANOL-d₄) δ ppm 8.17 (s, 1H) 7.92 (s, 1H) 7.70 (d, J=8.34 Hz, 1H) 7.40-7.54 (m, 2H) 7.32 (d, J=7.70 Hz, 1H) 7.09-7.26 (m, 3H) 6.96 (d, J=7.83 Hz, 1H) 6.87 (td, J=7.39, 1.01 Hz, 1H) 5.06-5.27 (m, 2H) 4.52 (t, J=6.00 Hz, 1H) 4.27-4.43 (m, 2H) 3.79-4.03 (m, 2H) 3.48-3.68 (m, 2H) 2.74-2.89 (m, 2H) 1.95-2.20 (m, 2H). HRMS calcd. for C₂₆H₂₈FNO₄ (M+H)⁺ 438.2003. found 438.1974.

### Example 273

### Example 273-A

### Methyl 2-(2-((5-bromo-2-methylbenzyl)oxy)phenyl)acetate

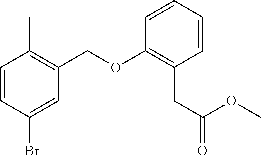 [SMILES: COC(=O)Cc1ccccc1OCc1cc(Br)ccc1C]

The title compound was synthesized as described in Intermediate 35 starting with (5-bromo-2-methylphenyl)methanol (CAS #25886-04-3) and methyl 2-(2-hydroxyphenyl)acetate (CAS #22446-37-3). ¹H NMR (400 MHz, DMSO-d₆) δ ppm 7.45-7.52 (m, 2H) 7.21-7.30 (m, 2H) 6.98-7.06 (m, 2H) 6.93 (t, J=7.45 Hz, 1H) 5.04 (s, 2H) 3.83 (s, 3H) 3.66 (s, 2H) 2.50 (s, 3H).

### Example 273-B

### (R)-2-(2-((3′-(1-aminoethyl)-2′-fluoro-4-methyl-[1,1′-biphenyl]-3-yl)methoxy)phenyl)acetic acid

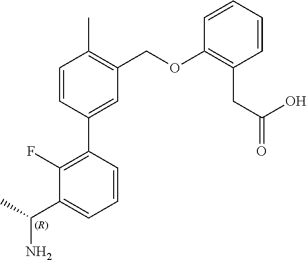

The title compound was synthesized from Example 273-A as described in Example 210. ¹H NMR (400 MHz, METHANOL-d₄) δ ppm 7.79 (s, 1H) 7.39-7.48 (m, 2H) 7.33-7.38 (m, 1H) 7.25-7.31 (m, 2H) 7.19-7.25 (m, 1H) 7.12-7.18 (m, 1H) 6.97 (d, J=8.08 Hz, 1H) 6.84-6.91 (m, 1H) 5.16 (s, 2H) 4.57-4.65 (m, 1H) 3.56 (s, 2H) 2.42 (s, 3H) 1.59 (d, J=6.82 Hz, 3H). HRMS calcd. for C₂₄H₂₄FNO₃ (M+H)⁺ 394.1818. found 394.1813.

### Example 274

### (S)-2-(2-((3′-(1-aminoethyl)-2′-fluoro-4-methyl-[1,1′-biphenyl]-3-yl)methoxy)phenyl)acetic acid

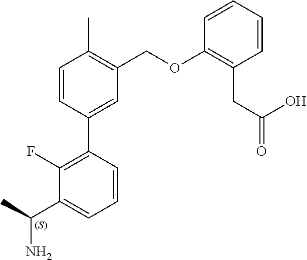

The title compound was synthesized as described in Example 273. ¹H NMR (400 MHz, METHANOL-d4) δ ppm 7.85 (br. s., 1H) 7.39-7.51 (m, 2H) 7.25-7.37 (m, 3H) 7.12-7.22 (m, 2H) 6.96 (d, J=8.84 Hz, 1H) 6.84-6.92 (m, 1H) 5.16 (s, 2H) 4.64-4.72 (m, 1H) 3.57 (s, 2H) 2.41 (s, 3H) 1.50-1.78 (m, 3H). HRMS calcd. for C₂₄H₂₄FNO₃ (M+H)⁺ 394.1818. found 394.1829.

### Example 275

### (R)-2-(2-((3′-(1-aminoethyl)-4-chloro-2′-fluoro-[1,1′-biphenyl]-3-yl)methoxy)phenyl)acetic acid

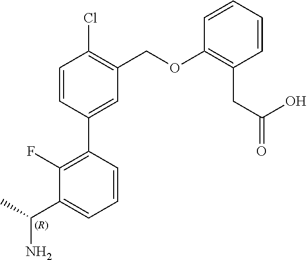

The title compound was synthesized as described in Example 273. ¹H NMR (400 MHz, METHANOL-d4) δ ppm 7.97 (s, 1H) 7.49-7.53 (m, 1H) 7.43-7.49 (m, 3H) 7.25-7.31 (m, 1H) 7.21 (d, J=7.58 Hz, 1H) 7.12-7.18 (m, 1H) 6.86-6.95 (m, 2H) 5.26 (s, 2H) 4.57-4.64 (m, 1H) 3.58 (s, 2H) 1.58 (d, J=6.82 Hz, 3H). HRMS calcd. for C₂₃H₂₁ClFNO₃ (M+H)⁺ 414.1272. found 414.1281.

### Example 276

### (R)-2-(2-((3′-(1-aminoethyl)-2′-fluoro-4-methoxy-[1,1′-biphenyl]-3-yl)methoxy)phenyl)acetic acid

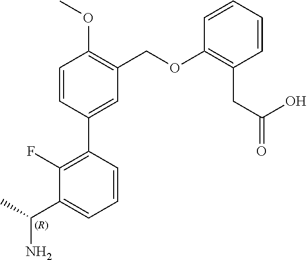

The title compound was synthesized as described in Example 273. ¹H NMR (400 MHz, METHANOL-d₄) δ ppm 7.87 (br. s., 1H) 7.35-7.50 (m, 3H) 7.22-7.29 (m, 1H) 7.19 (d, J=7.45 Hz, 1H) 7.06-7.16 (m, 2H) 6.82-6.92 (m, 2H) 5.18 (s, 2H) 4.64-4.71 (m, 1H) 3.94 (s, 3H) 3.57 (s, 2H) 1.64 (d, J=7.07 Hz, 3H). HRMS calcd. for C₂₄H₂₄FNO₄ (M+H)⁺ 410.1767. found 410.1757.

### Example 277

### Example 277-A

### (S)-tert-butyl (1-(3-bromo-5-chlorophenyl)-2-hydroxyethyl)carbamate

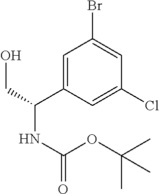 [SMILES: CC(C)(C)OC(=O)N[C@H](CO)c1cc(Cl)cc(Br)c1]

To a solution of (S)-2-amino-2-(3-bromo-5-chlorophenyl)ethanol (CAS #134417-25-9) (500 mg, 1.997 mmol) in dichloromethane (10.0 mL) was added di-tert butyl dicarbonate (544 mg, 2.46 mmol) and triethylamine (0.417 mL, 3.0 mmol). This mixture was stirred for several hours at room temperature followed by concentration in vacuo. The residue was partitioned between EtOAc and a cold 1.0 N HCl aqueous solution. The organic phase was washed with brine, dried over sodium sulfate and concentrated in vacuo. The residue was purified by flash column chromatography on silica gel (heptanes/EtOAc=100:0 to 70:30) to afford the title compound. ¹H NMR (400 MHz, CHLOROFORM-d) δ ppm 7.44 (t, J=1.71 Hz, 1H) 7.35 (s, 1H) 7.20-7.29 (m, 1H) 5.30 (t, J=3.28 Hz, 1H) 4.70 (br. s., 1H) 3.70-3.93 (m, 2H) 1.90 (br. s., 1H) 1.44 (s, 9H).

### Example 277-B

### (S)-Methyl 2-(2-((3′-(1-((tert-butoxycarbonyl)amino)-2-hydroxyethyl)-5′-chloro-[1,1′-biphenyl]-3-yl)methoxy)phenyl)acetate

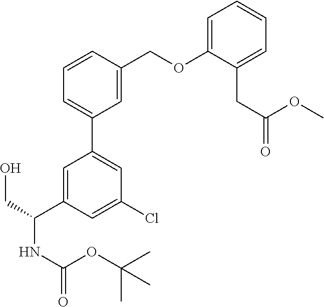 [SMILES: COC(=O)Cc1ccccc1OCc1cccc(-c2cc(Cl)cc([C@@H](CO)NC(=O)OC(C)(C)C)c2)c1]

A mixture of methyl 2-(2-((3-(4,4,5,5-tetramethyl-1,3,2-dioxaborolan-2-yl)benzyl)oxy)phenyl)acetate (Example 101-B) (181 mg, 0.472 mmol) and (S)-tert-butyl (1-(3-bromo-5-chlorophenyl)-2-hydroxyethyl)carbamate (138 mg, 0.394 mmol) in CH₃CN (2.0 mL) with 2.0M K₃PO₄ solution (0.69 mL, 1.377 mmol) was degassed with N₂(g) for 10 min and then PdCl₂(dppf).CH₂Cl₂ adduct (CAS #95464-05-4) (16.0 mg, 0.020 mmol) was added and the mixture heated at 90° C. for 2.0 hours and then cooled to r.t. The crude reaction mixture was filtered over a Celite pad and the filtrate was diluted with EtOAc. The organic phase was washed with water and brine, dried over Na₂SO₄ and concentrated in vacuo. The crude material was purified by flash column chromatography on silica gel (heptane/EtOAc=100:0 to 30:70) to give the title compound. ¹H NMR (400 MHz, METHANOL-d4) δ ppm 7.71-7.77 (m, 1H) 7.54-7.60 (m, 3H) 7.41-7.50 (m, 2H) 7.34 (s, 1H) 7.18-7.28 (m, 2H) 7.04 (d, J=8.08 Hz, 1H) 6.89-6.95 (m, 1H) 5.17 (s, 2H) 4.70 (br. s., 1H) 3.65-3.77 (m, 2H) 3.69 (s, 2H) 3.54 (s, 3H) 1.43 (br. s., 9H).

### Example 277-C

### (S)-2-(2-((3′-(1-Amino-2-hydroxyethyl)-5′-chloro-[1,1′-biphenyl]-3-yl)methoxy)phenyl)acetic acid

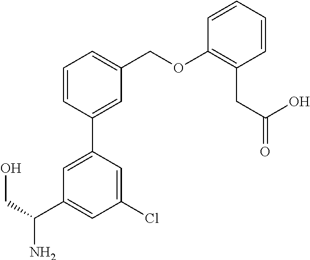 [SMILES: N[C@H](CO)c1cc(Cl)cc(-c2cccc(COc3ccccc3CC(=O)O)c2)c1]

To a solution of (S)-methyl 2-(2-((3′-(1-((tert-butoxycarbonyl)amino)-2-hydroxyethyl)-5′-chloro-[1,1′-biphenyl]-3-yl)methoxy)phenyl)acetate (100 mg, 0.190 mmol) in dichloromethane (1.0 mL) was added 4.0M HCl in dioxane (5.0 mL). This mixture was stirred for several hours and then concentrated in vacuo. The resulting material was then taken up in MeOH (1.0 mL) and 2.0 M LiOH solution (0.470 mL, 0.939 mmol) was added and this mixture was heated at 60° C. in an oil bath for 2.0 h. The reaction mixture was cooled to room temperature and concentrated in vacuo. The resulting residue was purified by HPLC (method B) to afford the title compound. ¹H NMR (400 MHz, METHANOL-d₄) δ ppm 8.01 (br. s., 2H) 7.37-7.69 (m, 5H) 7.10-7.26 (m, 2H) 6.96 (d, J=7.45 Hz, 1H) 6.87 (td, J=7.39, 1.01 Hz, 1H) 5.16-5.23 (m, 2H) 4.20-4.31 (m, 1H) 3.78-3.89 (m, 2H) 3.51-3.66 (m, 2H). HRMS calcd. for C₂₃H₂₂ClNO₄ (M+H)⁺ 412.1315. found 412.1330.

### Example 278

### Example 278-A

### tert-Butyl (1-(3-bromophenyl)-3-fluoropropyl)carbamate

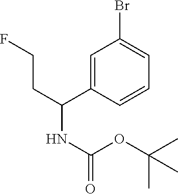 [SMILES: CC(C)(C)OC(=O)NC(CCF)c1cccc(Br)c1]

To a solution of tert-butyl (1-(3-bromophenyl)-3-hydroxypropyl)carbamate (CAS #924817-81-2) (727 mg, 2.202 mmol) was added perfluoro-1-butanesulfonyl fluoride (CAS #375-72-4) (1.33 g, 4.40 mmol), diisopropylethylamine trihydrofluoride (CAS #131600-43-6) (625 mg, 3.30 mmol) and diisopropylethylamine (1.28 g, 9.91 mmol). This mixture was sealed and heated at 45 C overnight. The reaction was partitioned EtOAc/H₂O and the layers separated. The organic layer was washed with saturated sodium bicarbonate solution, brine solution, dried over Na₂SO₄ and concentrated in vacuo. The residue was purified by flash column chromatography on silica gel (heptanes/EtOAc=100:0 to 60:40) to afford the title compound. ¹H NMR (400 MHz, CHLOROFORM-d) δ ppm 7.38-7.44 (m, 2H) 7.22 (d, J=4.67 Hz, 2H) 5.02 (br. s., 1H) 4.75-4.93 (m, 1H) 4.32-4.61 (m, 2H) 1.98-2.24 (m, 2H) 1.42 (br. s., 9H).

### Example 278-B

### 2-(2-((3′-(1-amino-3-fluoropropyl)-[1,1′-biphenyl]-3-yl)methoxy)phenyl)acetic acid

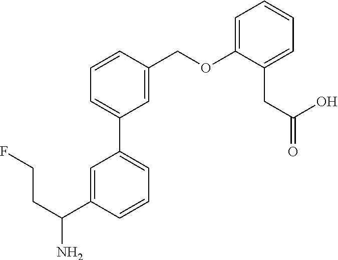 [SMILES: NC(CCF)c1cccc(-c2cccc(COc3ccccc3CC(=O)O)c2)c1]

The title compound was synthesized as described in Example 277. ¹H NMR (400 MHz, DMSO-d₆) δ ppm 9.83 (br. s., 1H), 8.06 (br. s., 1H) 7.91-8.00 (m, 1H) 7.62 (t, J=6.51 Hz, 2H) 7.36-7.49 (m, 3H) 7.24-7.33 (m, 1H) 7.04-7.18 (m, 2H) 6.95 (d, J=8.08 Hz, 1H) 6.81 (t, J=7.33 Hz, 1H) 5.15 (s, 2H) 4.22-4.66 (m, 3H) 4.06 (t, J=7.14 Hz, 1H) 3.38-3.42 (m, 1H) 3.17 (s, 2H) 2.01-2.26 (m, 2H). HRMS calcd. for C₂₄H₂₄FNO₃ (M+H)⁺ 394.1818. found 394.1808.

### Example 279

### Example 279-A

### (R)-tert-butyl (1-(3-bromophenyl)-3-fluoropropyl)carbamate

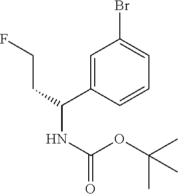

The title compound was synthesized as described in Example 278-A starting with (R)-tert-Butyl (1-(3-bromophenyl)-3-hydroxypropyl)carbamate (Intermediate 30-A). ¹H NMR (400 MHz, CHLOROFORM-d) δ ppm 7.38-7.44 (m, 2H) 7.22 (d, J=4.55 Hz, 2H) 5.01 (br. s., 1H) 4.84 (br. s., 1H) 4.31-4.62 (m, 2H) 2.04-2.27 (m, 2H) 1.41 (br. s., 9H).

### Example 279-B

### (R)-2-(2-((3′-(1-amino-3-fluoropropyl)-5-chloro-[1,1′-biphenyl]-3-yl)methoxy)phenyl)acetic acid

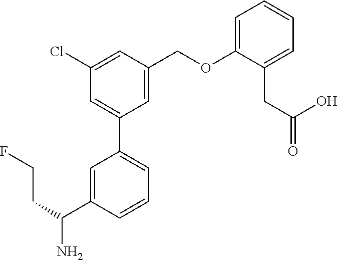

The title compound was synthesized as described in Example 277. ¹H NMR (400 MHz, METHANOL-d₄) δ ppm 8.27 (s, 1H) 8.02 (s, 1H) 7.71 (d, J=8.46 Hz, 1H) 7.63 (s, 1H) 7.49 (t, J=7.71 Hz, 1H) 7.41 (s, 1H) 7.35 (d, J=7.58 Hz, 1H) 7.13-7.22 (m, 2H) 6.96 (d, J=7.83 Hz, 1H) 6.84-6.92 (m, 1H) 5.08-5.28 (m, 2H) 4.42-4.65 (m, 2H) 4.17-4.38 (m, 1H) 3.49-3.69 (m, 2H) 2.26-2.65 (m, 2H). HRMS calcd. for C₂₄H₂₃ClFNO₃ (M+H)⁺ 428.1429. found 428.1413.

### Example 280

### (R)-2-(2-((3′-(1-aminoethyl)-4-cyclopropyl-2′-fluoro-[1,1′-biphenyl]-3-yl)methoxy)phenyl)acetic acid

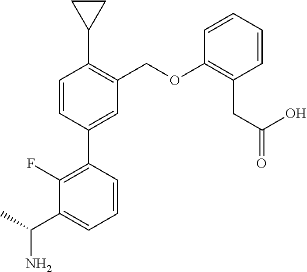 [SMILES: C[C@@H](N)c1cccc(-c2ccc(C3CC3)c(COc3ccccc3CC(=O)O)c2)c1F]

The title compound was synthesized as described in Example 277. ¹H NMR (400 MHz, METHANOL-d₄) δ ppm 7.87 (br. s., 1H) 7.39-7.50 (m, 2H) 7.36 (d, J=7.83 Hz, 1H) 7.24-7.30 (m, 1H) 7.12-7.22 (m, 3H) 6.96 (d, J=8.34 Hz, 1H) 6.83-6.90 (m, 1H) 5.37 (s, 2H) 4.63-4.73 (m, 1H) 3.53-3.65 (m, 2H) 1.97-2.12 (m, 1H) 1.64 (d, J=6.95 Hz, 3H) 0.97-1.12 (m, 2H) 0.66-0.78 (m, 2H). HRMS calcd. for C₂₆H₂₆FNO₃ (M+H)⁺ 420.1975. found 420.1959.

### Example 281

### Example 281-A

### Ethyl 3-bromo-5-(2,2-difluorocyclopropanecarboxamido)benzoate

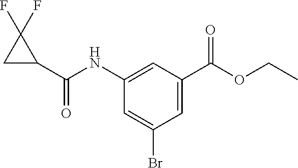 [SMILES: CCOC(=O)c1cc(Br)cc(NC(=O)C2CC2(F)F)c1]

To a solution of 2,2-difluorocyclopropanecarboxylic acid (CAS #847926-81-2) (149 mg, 1.221 mmol) was added HATU (CAS #148893-10-1) (535 mg, 1.408 mmol) in DMF (4.0 mL) and the mixture was stirred for 10 min at r.t. Then ethyl 3-amino-5-bromobenzoate (CAS #690260-95-8) and diisopropylethylamine (0.687 mL, 3.94 mmol) were added and this mixture was stirred at r.t. overnight. The reaction was diluted with EtOAc/H₂O and extracted; the organic phase was brine washed (3×), dried over Na₂SO₄, and concentrated in vacuo to afford the crude product. The residue was purified by flash column chromatography on silica gel (heptanes/EtOAc=100:0 to 60:40) to afford the title compound. MS (ESI+) m/z 348.0 350.0 (M+H).

### Example 281-B

### (3-bromo-5-(((2,2-difluorocyclopropyl)methyl)amino)phenyl)methanol

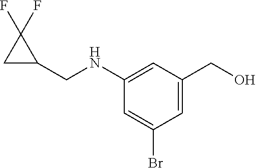 [SMILES: OCc1cc(Br)cc(NCC2CC2(F)F)c1]

To a solution of ethyl 3-bromo-5-(2,2-difluorocyclopropanecarboxamido)benzoate (240 mg, 0.689 mmol) was added borane-dimethylsulfide complex (CAS #13292-87-0) (1.0 M solution in THF, 3.45 mL, 3.45 mmol) at r.t. This mixture was stirred at r.t for 1.0 hr and then refluxed for 2.0 hr and then cooled to r.t. The reaction was carefully quenched dropwise with 1.0 N HCl solution; upon cessation of gaseous evolution, the reaction was basified with 2.0N NaOH solution to pH 9-10 and then partitioned EtOAc/H₂O and extracted. The organic phase was washed with saturated sodium bicarbonate solution, brine, dried over Na₂SO₄ and concentrated in vacuo to afford the crude product. The residue was purified by flash column chromatography on silica gel (heptanes/EtOAc=100:0 to 50:50) to afford the title compound. ¹H NMR (400 MHz, CHLOROFORM-d) δ ppm 6.79-6.90 (m, 1H), 6.64-6.73 (m, 1H), 6.50-6.60 (m, 1H), 4.54-4.62 (m, 2H), 3.27-3.41 (m, 1H), 3.13-3.25 (m, 1H), 2.08-2.28 (m, 1H), 1.82-1.99 (m, 1H), 1.66 (t, J=18.57 Hz, 1H), 1.50 (tdd, J=11.72, 11.72, 7.71, 4.36 Hz, 1H) and 1.05-1.20 (m, 1H).

### Example 281-C

### tert-Butyl 2-(2-((3-bromo-5-(((2,2-difluorocyclopropyl)methyl)amino)benzyl)oxy)phenyl)acetate

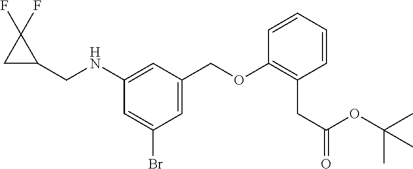 [SMILES: CC(C)(C)OC(=O)Cc1ccccc1OCc1cc(Br)cc(NCC2CC2(F)F)c1]

The title compound was synthesized as described in Intermediate 55 starting with (3-bromo-5-(((2,2-difluorocyclopropyl)methyl)amino)phenyl)methanol and tert-Butyl 2-(2-hydroxyphenyl)acetate (Intermediate 21). MS (ESI+) m/z 484.1 486.2 (M+H).

### Example 281-D

### tert-Butyl 2-(2-((3′-((R)-1-((tert-butoxycarbonyl)amino)ethyl)-5-(((2,2-difluorocyclopropyl)methyl)amino)-2′-fluoro-[1,1′-biphenyl]-3-yl)methoxy)phenyl)acetate

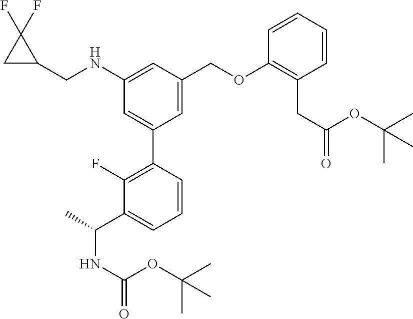 [SMILES: C[C@@H](NC(=O)OC(C)(C)C)c1cccc(-c2cc(COc3ccccc3CC(=O)OC(C)(C)C)cc(NCC3CC3(F)F)c2)c1F]

To a solution of tert-Butyl 2-(2-((3-bromo-5-(((2,2-difluorocyclopropyl)methyl)amino)benzyl)oxy)phenyl)acetate (110 mg, 0.228 mmol) and (R)-tert-butyl (1-(2-fluoro-3-(4,4,5,5-tetramethyl-1,3,2-dioxaborolan-2-yl)phenyl)ethyl)carbamate (Intermediate 27-B) (104 mg, 0.285 mmol) in DMF (2.0 mL) was added a 2.0M solution of K₃PO₄ (0.570 mL, 1.140 mmol); this mixture was degassed for 10 minutes with N₂(g), and then PdCl₂(dppf).CH₂Cl₂ adduct (9.3 mg, 0.011 mmol) added. The reaction was sealed and heated at 110° C. in an oil bath for 1.0 hr. The reaction was cooled to room temperature, filtered over a plug of Celite and the filtrate was partitioned between EtOAc/H₂O and the layers separated; the organic phase was washed with brine, dried over Na₂SO₄, and concentrated in vacuo. The residue was purified by flash column chromatography on silica gel (heptanes/EtOAc=100:0 to 60:40) to afford the title compound. MS (ESI+) m/z 663.3 (M+Na).

### Example 281-E

### 2-(2-((3′-((R)-1-Aminoethyl)-5-(((2,2-difluorocyclopropyl)methyl)amino)-2′-fluoro-[1,1′-biphenyl]-3-yl)methoxy)phenyl)acetic acid

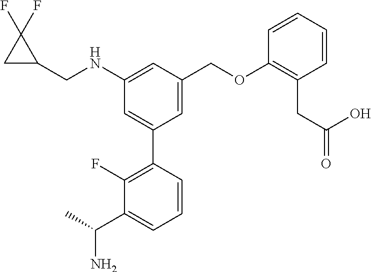 [SMILES: C[C@@H](N)c1cccc(-c2cc(COc3ccccc3CC(=O)O)cc(NCC3CC3(F)F)c2)c1F]

To a solution of tert-butyl 2-(2-((3′-((R)-1-((tert-butoxycarbonyl)amino)ethyl)-5-(((2,2-difluorocyclopropyl)methyl)amino)-2′-fluoro-[1,1′-biphenyl]-3-yl)methoxy)phenyl)acetate (100 mg, 0.156 mmol) from in dichloromethane (1.0 mL) was added 4.0M HCl in dioxane (5.0 mL). This mixture was stirred for 4 hours and then concentrated in vacuo. The resulting residue was purified by HPLC (method B) to afford the title compound. ¹H NMR (400 MHz, METHANOL-d₄) δ ppm 7.51 (td, J=7.55, 1.83 Hz, 1H) 7.39-7.46 (m, 1H) 7.26-7.32 (m, 1H) 7.07-7.23 (m, 3H) 6.93 (d, J=7.96 Hz, 1H) 6.85 (td, J=7.42, 0.95 Hz, 1H) 6.74-6.81 (m, 1H) 6.65-6.72 (m, 1H) 5.09 (s, 2H) 4.72 (q, J=6.99 Hz, 1H) 3.57 (s, 2H) 3.25-3.36 (m, 2H) 1.91-2.04 (m, 1H) 1.63-1.71 (m, 3H) 1.50 (tdd, J=11.84, 11.84, 7.71, 4.36 Hz, 1H) 1.15-1.26 (m, 1H). HRMS calcd. for C₂₇H₂₇F₃N₂O₃(M+H)⁺ 485.2052. found 485.2071.

### Example 282

### Example 282-A

### tert-Butyl 2-(2-((2-bromo-5-chlorobenzyl)oxy)phenyl)acetate

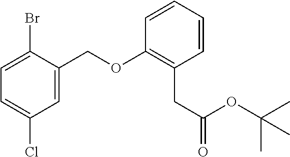 [SMILES: CC(C)(C)OC(=O)Cc1ccccc1OCc1cc(Cl)ccc1Br]

The title compound was synthesized as described in Intermediate 55 starting with (2-bromo-5-chlorophenyl)methanol (CAS #60666-70-8) and tert-Butyl 2-(2-hydroxyphenyl)acetate (Intermediate 21). ¹H NMR (400 MHz, CHLOROFORM-d) δ ppm 7.62 (d, J=2.53 Hz, 1H) 7.49 (d, J=8.46 Hz, 1H) 7.21-7.28 (m, 2H) 7.17 (dd, J=8.46, 2.53 Hz, 1H) 6.97 (t, J=7.45 Hz, 1H) 6.91 (d, J=8.08 Hz, 1H) 5.09 (s, 2H) 3.65 (s, 2H) 1.42 (s, 9H).

### Example 282-B

### tert-Butyl 2-(2-((5-chloro-2-vinylbenzyl)oxy)phenyl)acetate

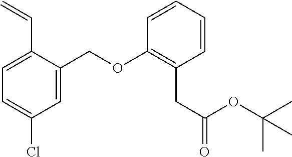 [SMILES: C=Cc1ccc(Cl)cc1COc1ccccc1CC(=O)OC(C)(C)C]

To a solution of tert-butyl 2-(2-((2-bromo-5-chlorobenzyl)oxy)phenyl)acetate (600 mg, 1.457 mmol) in DMF (7.0 mL) was added tributyl(vinyl)stannane (CAS #7486-35-3) (485 mg, 1.530 mmol). This mixture was degassed for 10 minutes with N₂(g), and then tetrakis(triphenylphosphine)palladium(0) (CAS #14221-01-3) (113 mg, 0.098 mmol) added. This mixture was heated in a sealed vessel at 120° C. overnight. The reaction was filtered over a pad of Celite with 50 wt % KF, partitioned with EtOAc/H₂O and extracted. The organic phase was washed with brine, dried over Na₂SO₄ and concentrated in vacuo to afford a light yellow oil as crude product. The residue was purified by flash column chromatography on silica gel (heptanes/EtOAc=100:0 to 70:30) to afford the title compound. ¹H NMR (400 MHz, CHLOROFORM-d) δ ppm 7.49 (d, J=2.20 Hz, 1H) 7.46 (d, J=8.31 Hz, 1H) 7.27-7.31 (m, 1H) 7.19-7.25 (m, 2H) 6.84-6.98 (m, 3H) 5.66 (dd, J=17.36, 1.10 Hz, 1H) 5.37 (dd, J=10.94, 1.16 Hz, 1H) 5.06 (s, 2H) 3.58 (s, 2H) 1.38 (s, 9H).

### Example 282-C

### Tert-butyl 2-(2-((5-chloro-2-ethylbenzyl)oxy)phenyl)acetate

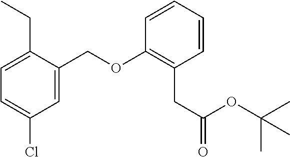 [SMILES: CCc1ccc(Cl)cc1COc1ccccc1CC(=O)OC(C)(C)C]

A solution of tert-butyl 2-(2-((5-chloro-2-vinylbenzyl)oxy)phenyl)acetate (142 mg, 0.396 mmol) in 1:1 iPrOH/toluene (4.0 mL) was degassed with N₂(g) for 10 min. Then Cs₂CO₃ (12.9 mg, 0.040 mmol), bis(1,5-cyclooctadiene)diiridium(I)dichloride (CAS #12112-67-3) (26.6 mmol, 0.040 mmol) and 1,3-bis(diphenylphosphino)propane (CAS #6737-42-4) (16.3 mg, 0.040 mmol) were added and this mixture was sealed and heated at 80° C. overnight. The reaction was filtered over a plug of Celite and then concentrated in vacuo. The residue was purified by flash column chromatography on silica gel (heptanes/EtOAc=100:0 to 75:25) to afford the title compound. ¹H NMR (400 MHz, CHLOROFORM-d) δ ppm 7.48 (d, J=2.15 Hz, 1H) 7.22-7.29 (m, 3H) 7.14-7.21 (m, 1H) 6.91-6.99 (m, 2H) 5.03 (s, 2H) 3.59 (s, 2H) 2.66 (q, J=7.58 Hz, 2H) 1.38 (s, 9H) 1.23 (t, J=7.52 Hz, 3H).

### Example 282-D

### (R)-2-(2-((3′-(1-aminoethyl)-4-ethyl-2′-fluoro-[1,1′-biphenyl]-3-yl)methoxy)phenyl)acetic acid

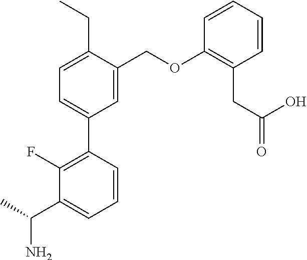 [SMILES: CCc1ccc(-c2cccc([C@@H](C)N)c2F)cc1COc1ccccc1CC(=O)O]

The title compound was synthesized in a similar manner as in Example 277-B and 277-C. ¹H NMR (400 MHz, METHANOL-d₄) δ ppm 7.91 (br. s., 1H) 7.51 (td, J=7.55, 1.71 Hz, 1H) 7.37-7.46 (m, 2H) 7.25-7.36 (m, 2H) 7.11-7.22 (m, 2H) 6.95 (d, J=7.71 Hz, 1H) 6.84-6.90 (m, 1H) 5.22 (s, 2H) 4.67-4.77 (m, 1H) 3.57 (s, 2H) 2.78 (q, J=7.66 Hz, 2H) 1.67 (d, J=6.95 Hz, 3H) 1.30 (t, J=7.52 Hz, 3H). HRMS calcd. for C₂₅H₂₆FNO₃ (M+H)⁺ 408.1975. found 408.1970.

### Example 283

### (R)-2-(2-((3′-(1-aminoethyl)-2′-fluoro-4,5-dimethyl-[1,1′-biphenyl]-3-yl)methoxy)phenyl)acetic acid

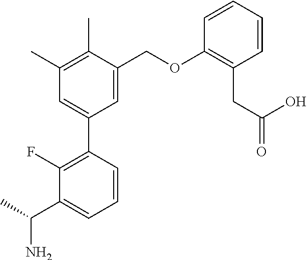 [SMILES: Cc1cc(-c2cccc([C@@H](C)N)c2F)cc(COc2ccccc2CC(=O)O)c1C]

The title compound was synthesized as described in Example 273. ¹H NMR (400 MHz, METHANOL-d₄) δ ppm 7.59 (br. s., 1H) 7.38-7.45 (m, 2H) 7.19-7.29 (m, 3H) 7.12-7.18 (m, 1H) 6.98 (d, J=7.96 Hz, 1H) 6.84-6.89 (m, 1H) 5.15 (s, 2H) 4.69-4.79 (m, 1H) 3.55 (s, 2H) 2.38 (s, 3H) 2.32 (s, 3H) 1.56 (d, J=5.81 Hz, 3H). HRMS calcd. for C₂₅H₂₆FNO₃ (M+H)⁺ 408.1975. found 408.1976.

### Example 284

### Example 284-A

### (S)-methyl 2-(2-((3′-(1-amino-2-fluoroethyl)-5-(((2,2,2-trifluoroethyl)amino)methyl)-[1,1′-biphenyl]-3-yl)methoxy)phenyl)acetate

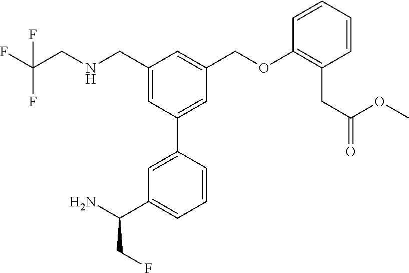 [SMILES: COC(=O)Cc1ccccc1OCc1cc(CNCC(F)(F)F)cc(-c2cccc([C@H](N)CF)c2)c1]

A mixture of PdCl₂(dppf) CH₂Cl₂ complex (83 mg, 0.102 mmol), potassium acetate (400 mg, 4.08 mmol) and 4,4,4′,4′,5,5,5′,5′-octamethyl-2,2′-bi(1,3,2-dioxaborolane) (388 mg, 1.529 mmol), Methyl 2-(2-((3-bromo-5-(((2,2,2-trifluoroethyl)amino)methyl)benzyl)oxy)phenyl)acetate (Intermediate 102-A, 430 mg, 0.964 mmol) in 3 ml of anhydrous dioxane was stirred under argon atmosphere for 12 h at 80° C. The mixture was cooled down to 25° C. and then diluted in 50 ml of EtOAc. The brown solution was then filtered over a pad of Celite, and the filtrate was concentrated. The brown residue obtained was then diluted in Cyclohexane (50 ml). The mixture was filtrated and the orange filtrate was concentrated and then dried under high vacuum to afford 980 mg of crude product. To a solution of this crude product (150 mg, 0.152 mmol) and (S)-1-(3-bromophenyl)-2-fluoroethanamine (Intermediate 29-C, 43.1 mg, 0.198 mmol) in 10 ml DMF was added K₃PO₄ (0.304 mL, 0.608 mmol) and S-Phos (3.12 mg, 7.60 μmol). The reaction was heated at 110° C. for 1 h 40 in the microwave oven.

The black mixture was diluted in EtOAc and washed twice with saturated bicarbonate solution. The organic layer was extracted, dried over sodium sulfate, filtered and concentrated to dryness. The residue was purified by flash-chromatography using Isolera One system: 25 g KP-SIL Biotage column of SiO₂, Mobil phase: Cyclohexane/EtOAc, Gradient: 0% to 100% in 31:55 min and 100% of EtOAc for 8 min, collection 254 nm, flow rate 25 ml/min to afford 24 mg of (S)-methyl 2-(2-((3′-(1-amino-2-fluoroethyl)-5-(((2,2,2-trifluoroethyl)amino)methyl)-[1,1′-biphenyl]-3-yl)methoxy)phenyl)acetate as a colorless oil. MS (ESI+) m/z 505.3 (M+H).

### Example 284-B

### (S)-2-(2-((3′-(1-amino-2-fluoroethyl)-5-(((2,2,2-trifluoroethyl)amino)methyl)-[1,1′-biphenyl]-3-yl)methoxy)phenyl)acetic acid

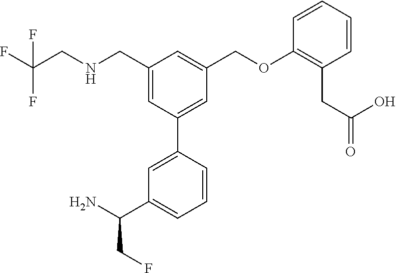 [SMILES: N[C@H](CF)c1cccc(-c2cc(CNCC(F)(F)F)cc(COc3ccccc3CC(=O)O)c2)c1]

To a solution of (S)-methyl 2-(2-((3′-(1-amino-2-fluoroethyl)-5-(((2,2,2-trifluoroethyl)amino)methyl)-[1,1′-biphenyl]-3-yl)methoxy)phenyl)acetate (24 mg, 0.043 mmol) in 0.5 ml THF and 0.1 mL water was added LiOH.H₂O (4.49 mg, 0.107 mmol). The reaction mixture was stirred at 25° C. for 12 h. The mixture was concentrated to dryness to afford a brown solid. The residue was taken up in 2 ml of 4M HCl/Dioxane 4M (2 ml), sonicated and then centrifuged and filtered. The orange filtrate was concentrated, then dissolved in ACN/Water and lyophilized overnight to afford 33 mg of a beige powder. The crude material was purified by supercritical fluid chromatography on the Princeton PPU column 100A-5 μm (flow rate 100 ml/min, gradient 31 to 35% DCM-MeOH in 10 min) to afford (S)-2-(2-((3′-(1-amino-2-fluoroethyl)-5-(((2,2,2-trifluoroethyl)amino)methyl)-[1,1′-biphenyl]-3-yl)methoxy)phenyl)acetic acid. MS (ESI+) m/z 491.2 (M+H). ¹H NMR (400 MHz, DMSO-d₆) δ 8.02 (s, 1H), 7.79 (s, 1H), 7.76-7.60 (m, 2H), 7.56-7.35 (m, 3H), 7.19 (d, J=7.6 Hz, 2H), 7.04 (d, J=8.1 Hz, 1H), 6.89 (t, J=7.4 Hz, 1H), 5.19 (s, 2H), 4.78 (t, J=8.5 Hz, 1H), 4.72-4.61 (m, 1H), 4.55 (dd, J=9.3, 4.7 Hz, 1H), 4.45 (ddd, J=12.9, 7.6, 4.9 Hz, 1H), 3.89 (s, 2H), 3.54 (s, 2H), 3.24 (q, J=10.2 Hz, 2H).

### Example 285

The following compounds were prepared in analogy to Example 284:

| | | MS (ESI+) | |
|---|---|---|---|
| Ex. | Structure/Chemical Name | m/z (M + H) | ¹H NMR (400 MHz, DMSO-d₆) δ |
| 285-A | 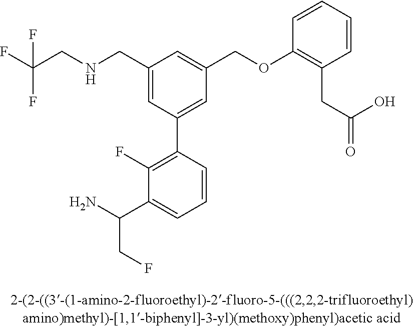 [SMILES: NC(CF)c1cccc(-c2cc(CNCC(F)(F)F)cc(COc3ccccc3CC(=O)O)c2)c1F] | 509.2 | 7.59-7.49 (m, 1H), 7.49- 7.35 (m, 4H), 7.27 (t, J = 7.7 Hz, 1H), 7.22-7.09 (m, 2H), 6.99 (d, J = 8.1 Hz, 1H), 6.84 (t, J = 7.4 Hz, 1H), 5.11 (s, 2H), 4.57 (tdd, J = 12.2, 8.1, 5.7 Hz, 2H), 4.51-4.37 (m, 2H), 3.80 (s, 2H), 3.51 (s, 2H), 3.14 (q, J = 10.1 Hz, 2H). |
| 285-B | 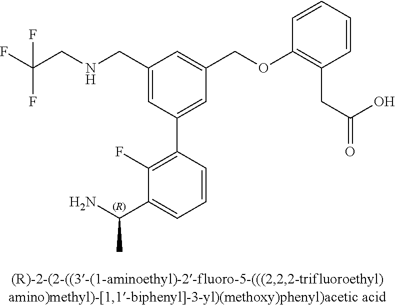 | 491.3 | 7.62-7.25 (m, 6H), 7.14 (d, J = 7.8 Hz, 2H), 6.98 (d, J = 8.1 Hz, 1H), 6.83 (t, J = 7.4 Hz, 1H), 5.11 (s, 2H), 4.56 (q, J = 6.8 Hz, 1H), 3.80 (s, 2H), 3.50 (s, 2H), 3.15 (q, J = 10.2 Hz, 2H), 1.44 (d, J = 6.7 Hz, 3H). |

### Example 286

### Example 286-A

### Methyl 2-(2-((3-(aminomethyl)-5-bromobenzyl)oxy)phenyl)acetate

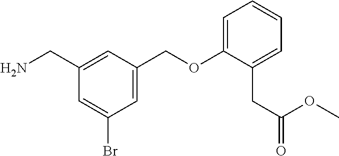 [SMILES: COC(=O)Cc1ccccc1OCc1cc(Br)cc(CN)c1]

To a solution of methyl 2-(2-((3-bromo-5-cyanobenzyl)oxy)phenyl)acetate (3.21 g, 8.47 mmol) in THF (68 ml)/H₂O (17 ml) was added cobalt(II) chloride hexahydrate (3.02 g, 12.7 mmol) at 0° C. NaBH₄ (1.95 g, 51 mmol) was portion wise added at 0° C. After 24 h at 0° ice-water, NaHCO₃-solution and CH₂Cl₂ (350 ml) were added to the black reaction mixture. The suspension was filtered through Hyflo Super Gel medium and the filtrate was extracted three times with CH₂Cl₂. The combined organic layers were dried over Na₂SO₄, filtered and concentrated under vacuum. The crude product was purified by flash-chromatography on ARMEN Spot Quod® (RediSep® R_{f}, Teledyne Isco, 120 g), gradient of CH₂Cl₂, 0.2% Et3N/CH₂Cl₂, MeOH 10% (80 ml/min.) from 100/0 (10 min.) to 90/10 (10-20 min.) to 0/100 (20-40 min.) to afford pure methyl 2-(2-((3-(aminomethyl)-5-bromobenzyl)oxy)phenyl)acetate (1.333 g) MS (ESI+) m/z 364.1, 366.1 (M+NH4).

### Example 286-B

### Methyl 2-(2-((3-bromo-5-(((2,2,2-trifluoro-1-phenylethyl)amino)methyl)benzyl)oxy)phenyl)acetate

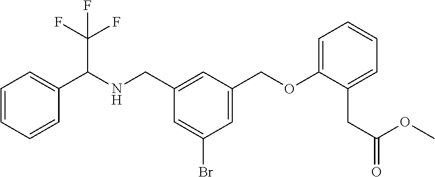 [SMILES: COC(=O)Cc1ccccc1OCc1cc(Br)cc(CNC(c2ccccc2)C(F)(F)F)c1]

A mixture of methyl 2-(2-((3-(aminomethyl)-5-bromobenzyl)oxy)phenyl)acetate (400 mg, 1.098 mmol), 2,2,2-trifluoro-1-phenylethanone (574 mg, 3.29 mmol) and molecular sieves 3A (480 mg) in toluene (9.6 ml), AcOH (1.3 ml) was heated at 90° C. for 8 h and subsequently stirred for 12 h at room temperature. The solution was separated from molecular sieves, diluted with toluene and evaporated. To the residue was added fresh molecular sieve, 2,2,2-trifluoro-1-phenylethanone (574 mg, 3.29 mmol) and AcOH (1.5 ml) and the mixture was heated again under reflux on a Dean-Stark-apparatus for 5 h. The reaction solution was concentrated under vacuum. The residue (660 mg) was dissolved in MeOH (10 ml) and NaCNBH₃ (414 mg, 6.59 mmol) was added at 23° C. The mixture was heated at 45° C. for 2.25 h. The reaction mixture was quenched with H2O/Na₂CO₃/brine and extracted twice with EtOAc. The combined organic layers were dried over Na₂SO₄, filtered and concentrated under vacuum. The crude product (660 mg, orange oil) was purified by flash-chromatography on ARMEN Spot Quod® (RediSep® R_{f}, Teledyne Isco, 80 g), gradient of Cyclohexane/EtOAc (80 ml/min.) from 100/0 (2 min.) to 70/30 (2-20 min.) to give the title compound. MS (ESI+) m/z 522.2, 524.2 (M+H).

### Example 286-C

### Methyl 2-(2-((3′-(((tert-butoxycarbonyl)amino)methyl)-5-(((2,2,2-trifluoro-1-phenylethyl)amino)methyl)-[1,1′-biphenyl]-3-yl)methoxy)phenyl)acetate

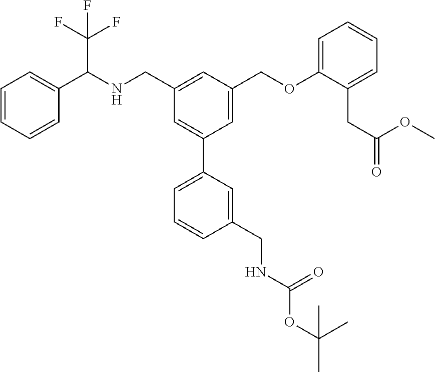 [SMILES: COC(=O)Cc1ccccc1OCc1cc(CNC(c2ccccc2)C(F)(F)F)cc(-c2cccc(CNC(=O)OC(C)(C)C)c2)c1]

To a solution of Methyl 2-(2-((3-bromo-5-(((2,2,2-trifluoro-1-phenylethyl)amino)methyl)benzyl)oxy)phenyl)acetate (100 mg, 0.191 mmol) in Dioxane (3 mL)/Water (1 mL) was added successively (3-(((tert-butoxycarbonyl)amino)methyl)phenyl)boronic acid (60.1 mg, 0.239 mmol), PdCl₂(PPh₃)₂ (5.37 mg, 7.66 μmol) and K₃PO₄*H₂O (110 mg, 0.479 mmol). The reaction mixture was stirred for 1 h at 100° C. The reaction solution was taken up in ethyl acetate and washed with aq. sat. NaHCO₃ solution. The water layer was extracted two times with ethyl acetate. The combined organic layer were dried with a phase separator and evaporated to dryness.

The crude material was taken up in 4 ml THF and added 4.0 eq. SiliaMetS Thiol Loading 1.39 mmol/g (30 mg). The suspension was stirred 1 h at 40° C. The suspension was filtered and the solution was evaporated to dryness. The crude product was purified by flash-chromatography (ISCO on 12 g silica gel), gradient of cyclohexane/EtOAc (30 ml/min.) from 100/0 to 80/20 (30 min.) to afford a colorless oil of methyl 2-(2-((3′-(((tert-butoxycarbonyl)amino)methyl)-5-(((2,2,2-trifluoro-1-phenylethyl)amino)methyl)-[1,1′-biphenyl]-3-yl)methoxy)phenyl)acetate (83 mg, 0.115 mmol). MS (ESI+) m/z 649.5 (M+H).

### Example 286-D

### 2-(2-((3′-(((tert-butoxycarbonyl)amino)methyl)-5-(((2,2,2-trifluoro-1-phenylethyl)amino)methyl)-[1,1′-biphenyl]-3-yl)methoxy)phenyl)acetic acid

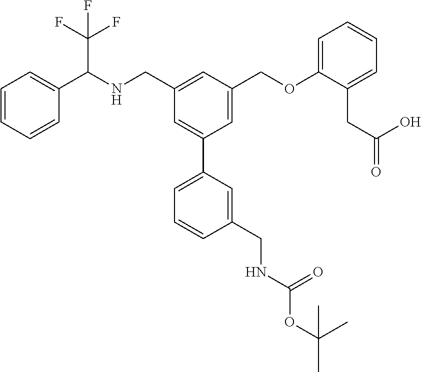 [SMILES: CC(C)(C)OC(=O)NCc1cccc(-c2cc(CNC(c3ccccc3)C(F)(F)F)cc(COc3ccccc3CC(=O)O)c2)c1]

To a solution of methyl 2-(2-((3′-(((tert-butoxycarbonyl)amino)methyl)-5-(((2,2,2-trifluoro-1-phenylethyl)amino)methyl)-[1,1′-biphenyl]-3-yl)methoxy)phenyl)acetate (83 mg, 0.128 mmol) in Tetrahydrofuran (3 mL)/Water (1.500 mL) was added LiOH. H₂O (13.42 mg, 0.320 mmol). The reaction mixture was stirred for 16 h at 23° C. The reaction mixture was taken up in ethyl acetate and washed with 0.25M HCl. The water layer was extracted two times with ethyl acetate. The combined organic layers were dried with a phase separator and evaporated to dryness to afford a colorless oil of 2-(2-((3′-(((tert-butoxycarbonyl)amino)methyl)-5-(((2,2,2-trifluoro-1-phenylethyl)amino)methyl)-[1,1′-biphenyl]-3-yl)methoxy)phenyl)acetic acid (81 mg).

MS (ESI+) m/z 635.5 (M+H).

### Example 286-E

### 2-(2-((3′-(aminomethyl)-5-(((2,2,2-trifluoro-1-phenylethyl)amino)methyl)-[1,1′-biphenyl]-3-yl)methoxy)phenyl)acetic acid

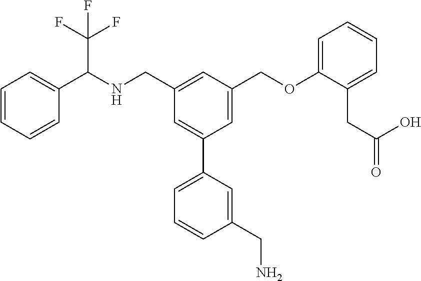 [SMILES: NCc1cccc(-c2cc(CNC(c3ccccc3)C(F)(F)F)cc(COc3ccccc3CC(=O)O)c2)c1]

To a solution of 2-(2-((3′-(((tert-butoxycarbonyl)amino)methyl)-5-(((2,2,2-trifluoro-1-phenylethyl)amino)methyl)-[1,1′-biphenyl]-3-yl)methoxy)phenyl)acetic acid (80 mg, 0.126 mmol) in Dichloromethane (2 mL) was added TFA (0.049 mL, 0.630 mmol). The reaction mixture was stirred for 2 h at 23° C. Additional TFA (0.049 mL, 0.630 mmol) was added to the reaction solution. After 3 h the solution was evaporated to dryness. The crude product was solved in CH₂Cl₂/MeOH and purified with supercritical fluid chromatography (Conditions: 250×30 Princeton PPU 100A 5 μm—FLOW 100 ml/min, Description: 26 to 35% in 10 min—(MeOH/DCM)) to afford the title compound. MS (ESI+) m/z 535.3 (M+H). ¹H NMR (400 MHz, DMSO-d₆) δ 8.20 (s, 1H), 7.89 (s, 1H), 7.68-7.50 (m, 4H), 7.43 (qd, J=6.7, 6.3, 2.3 Hz, 4H), 7.32 (d, J=8.8 Hz, 2H), 7.18-7.07 (m, 2H), 6.93 (d, J=8.0 Hz, 1H), 6.83 (t, J=7.3 Hz, 1H), 5.19 (s, 2H), 4.37 (q, J=8.3 Hz, 1H), 4.00 (s, 2H), 3.82 (d, J=14.0 Hz, 1H), 3.66 (d, J=14.1 Hz, 1H), 3.44 (s, 2H).

### Example 287-A

### Methyl 2-(2-((3-bromo-5-(hydroxymethyl)benzyl)oxy)phenyl)acetate

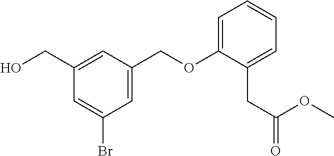 [SMILES: COC(=O)Cc1ccccc1OCc1cc(Br)cc(CO)c1]

To a solution of methyl 2-(2-hydroxyphenyl)acetate (3.5 g, 20 mmol), (5-bromo-1,3-phenylene)dimethanol (4.34 g, 20 mmol) and triphenylphosphine (5.51 g, 21 mmol) in THF (200 ml) was added drop wise DEAD (40% in toluene, 9.63 ml, 21 mmol) at 5° C. The ice-bath was removed and the pale orange solution was stirred at 23° C. for 5 hours. The reaction mixture was diluted with EtOAc (200 ml) and quenched with H2O/brine. The organic layer was separated and the aqueous layer extracted with EtOAc. The combined organic layers were dried over Na₂SO₄, filtered and concentrated under vacuum to afford an orange oil (20 g). The crude product was purified in 2 parts by flash-chromatography on ARMEN Spot Quod® (RediSep® R_{f}, Teledyne Isco, 120 g), gradient of cyclohexane/EtOAc (75 ml/min.) from 100/0 (4 min.) to 70/30 (5-50 min.) to afford the title compound. MS (ESI+) m/z 382.1, 384.1 (M+NH₄), TLC (EtOAc/cyclohexane=1:2) R_{f}0.32 (UV254 nm).

### Example 287-C

### 3-Bromo-5-((2-(2-methoxy-2-oxoethyl)phenoxy)methyl)benzoic acid

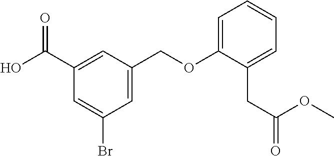 [SMILES: COC(=O)Cc1ccccc1OCc1cc(Br)cc(C(=O)O)c1]

To a solution of methyl 2-(2-((3-bromo-5-(hydroxymethyl)benzyl)oxy)phenyl)acetate (2220 mg, 5.96 mmol) in CH₃CN (35 ml) was added TEMPO (65.2 mg, 0.417 mmol), TBAHSO₄ (81 mg, 0.238 mmol) and phosphate-buffer (pH 6.8) (22.2 ml). The mixture was heated at 35° C. and a solution of NaClO₂ (80%, 1.347 g, 11.91 mmol) in H₂O (6.0 ml) was added simultaneously with NaOCl (4%, 0.4 ml). The pale yellow-orange color turned to pale red-brown. The mixture turned again to yellow and was stirred at 35° for 5 h. Additional 1 ml of NaOCl was added. The reaction mixture (pale yellow two layers mixture) was allowed to cool to RT, diluted with H₂O and Na₂SO₃ (1.6 g) was added. Then was acidified with 4 N HCl (2 ml) to pH 1-2. The mixture was extracted with EtOAc. The combined organic layers were washed with brine, dried over Na₂SO₄, filtered and concentrated under vacuum. The crude product was triturated in diethyl ether (12 ml), treated in an ultra-sonic bath for 2 min and filtered off to the title compound. MS (ESI+) m/z 378.9, 380.9 (M+H),

### Example 287-D

### (S)-3′-(1-((tert-butoxycarbonyl)amino)-2-hydroxyethyl)-5-((2-(2-methoxy-2-oxoethyl)phenoxy)methyl)-[1,1′-biphenyl]-3-carboxylic acid

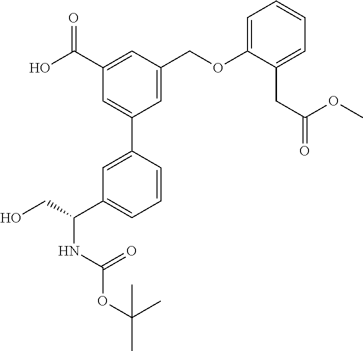 [SMILES: COC(=O)Cc1ccccc1OCc1cc(C(=O)O)cc(-c2cccc([C@@H](CO)NC(=O)OC(C)(C)C)c2)c1]

A mixture of 3-bromo-5-((2-(2-methoxy-2-oxoethyl)phenoxy)methyl)benzoic acid (400 mg, 1.055 mmol), (S)-tert-butyl (2-hydroxy-1-(3-(4,4,5,5-tetramethyl-1,3,2-dioxaborolan-2-yl)phenyl)ethyl)carbamate (536 mg, 1.477 mmol), K₃PO₄H₂O (729 mg, 3.16 mmol) and PdCl₂(PPh₃)₂, (37 mg, 0.053 mmol) in DME (4.0 ml)/H₂O (1.3 ml) was stirred at 85° C. for 1.75 h. The reaction mixture was quenched with H₂O/brine, acidified with 4 N HCl (pH 2) and filtered through a Chem-elut extraction cartridge with EtOAc and the filtrate was concentrated under vacuum. The crude product was purified by flash-chromatography on ARMEN Spot Quod® (RediSep® R_{f}, Teledyne Isco, 80 g), gradient of cyclohexane/[cyclohexane/EtOAc/AcOH=100/100/1] (75 ml/min.) from 100/0 (5 min.) to 0/100 (5-25 min.-45 min.) to afford the title compound. MS (ESI+) m/z 536.2 (M+H),

### Example 287-E

### Methyl 2-(2-((3′-((S)-1-((tert-butoxycarbonyl)amino)-2-hydroxyethyl)-5-((S)-chroman-4-ylcarbamoyl)-[1,1′-biphenyl]-3-yl)methoxy)phenyl)acetate

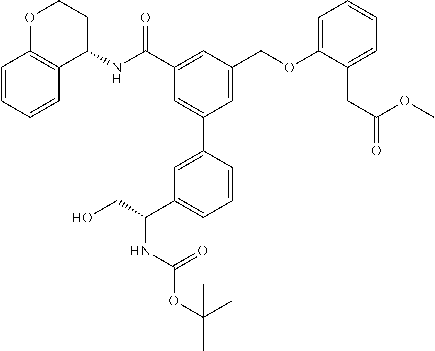 [SMILES: COC(=O)Cc1ccccc1OCc1cc(C(=O)N[C@H]2CCOc3ccccc32)cc(-c2cccc([C@@H](CO)NC(=O)OC(C)(C)C)c2)c1]

To a solution of (S)-3′-(1-((tert-butoxycarbonyl)amino)-2-hydroxyethyl)-5-((2-(2-methoxy-2-oxoethyl)phenoxy)methyl)-[1,1′-biphenyl]-3-carboxylic acid (71 mg, 0.125 mmol), (S)-chroman-4-amine (27.9 mg, 0.187 mmol) and DIPEA (65.3 ul, 0.374 mmol) in CH₂Cl₂ (1.3 ml) was added propylphosphonic anhydride solution (50% in DMF, 111 ul, 0.187 mmol) at 23° C. The reaction mixture was stirred at 23° C. for 4 h. The reaction mixture was quenched with H₂O/brine and extracted with twice with EtOAc. The combined organic layers were dried over Na₂SO₄, filtered and concentrated under vacuum to afford a yellow oil. The crude product was purified by flash-chromatography on ARMEN Spot Quod® (RediSep® R_{f}, Teledyne Isco, 12 g), gradient of cyclohexane/[cyclohexane/EtOAc=2:1] (40 ml/min.) from 100/0 (4 min.) to 0/100 (4-10 min. and hold for 20 min.) to afford the title compound. MS (ESI+) m/z 667.32 (M+H).

### Example 287-F

### 2-(2-((3′-((S)-1-((tert-butoxycarbonyl)amino)-2-hydroxyethyl)-5-((S)-chroman-4-ylcarbamoyl)-[1,1′-biphenyl]-3-yl)methoxy)phenyl)acetic acid

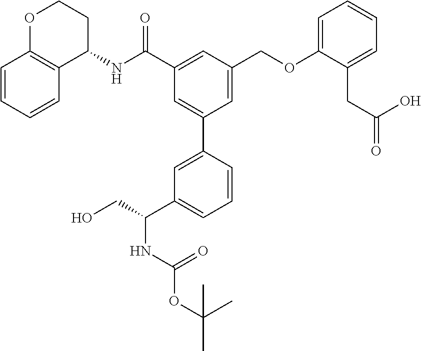 [SMILES: CC(C)(C)OC(=O)N[C@H](CO)c1cccc(-c2cc(COc3ccccc3CC(=O)O)cc(C(=O)N[C@H]3CCOc4ccccc43)c2)c1]

A mixture of methyl 2-(2-((3′-((S)-1-((tert-butoxycarbonyl)amino)-2-hydroxyethyl)-5-((S)-chroman-4-ylcarbamoyl)-[1,1′-biphenyl]-3-yl)methoxy)phenyl)acetate (61 mg, 0.091 mmol) and LiOH (9.8 mg, 0.41 mmol) in THF (1.3 ml)/H₂O (0.45 ml) was stirred at 35° C. for 9 h. The reaction mixture was concentrated under reduced pressure and acidified with 1 N HCl (0.45 ml). The resulting suspension was extracted with EtOAc, the organic layer was dried over Na₂SO₄, filtered and concentrated under vacuum to afford the title compound. MS (ESI+) m/z 653.3 (M+H),

### Example 287-G

### 2-(2-((3′-((S)-1-amino-2-hydroxyethyl)-5-((S)-chroman-4-ylcarbamoyl)-[1,1′-biphenyl]-3-yl)methoxy)phenyl)acetic acid

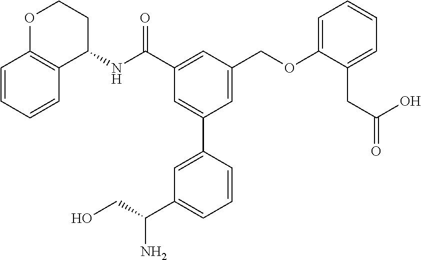 [SMILES: N[C@H](CO)c1cccc(-c2cc(COc3ccccc3CC(=O)O)cc(C(=O)N[C@H]3CCOc4ccccc43)c2)c1]

To a solution of 2-(2-((3′-((S)-1-((tert-butoxycarbonyl)amino)-2-hydroxyethyl)-5-((S)-chroman-4-ylcarbamoyl)-[1,1′-biphenyl]-3-yl)methoxy)phenyl)acetic acid (57.5 mg, 0.088 mmol) in dioxane (0.22 ml) was added HCl (4 N in dioxane, 0.22 ml) at 23° C. The reaction mixture was concentrated under reduced pressure, the residue was dissolved in H₂O and lyophilized to give the title compound. MS (ESI+) m/z 553.3 (M+H), ¹H NMR (400 MHz, DMSO-d₆) δ 9.03 (d, J=8.2 Hz, 1H), 8.20 (d, J=1.7 Hz, 1H), 8.03 (t, J=1.5 Hz, 1H), 8.00-7.89 (m, 2H), 7.80 (dt, J=7.7, 1.5 Hz, 1H), 7.61-7.45 (m, 2H), 7.32-7.12 (m, 4H), 7.07 (d, J=8.2 Hz, 1H), 6.98-6.80 (m, 3H), 5.58 (d, J=36.2 Hz, 1H), 5.42-5.31 (m, 1H), 5.26 (s, 2H), 4.45-4.23 (m, 3H), 3.86-3.71 (m, 2H), 3.62 (s, 2H), 2.14 (ddtt, J=17.4, 10.3, 7.0, 3.4 Hz, 2H).

### Example 288

### 2-(2-((3′-((methylamino)methyl)-[1,1′-biphenyl]-3-yl)methoxy)phenyl)acetic acid

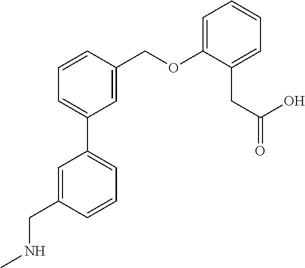 [SMILES: CNCc1cccc(-c2cccc(COc3ccccc3CC(=O)O)c2)c1]

To a mixture of methyl 2-(2-((3′-((methylamino)methyl)-[1,1′-biphenyl]-3-yl)methoxy)phenyl)acetate (Intermediate 163) (50 mg, 0.121 mmol) in MeOH (0.37 mL) and water (37 μL) NaOH (1N in water, 728 μL, 0.728 mmol) was added and the reaction was stirred at room temperature for 72 hours. The mixture was neutralized with 2N HCl (0.37 mL, 0.728 mmol), solvents were removed and the crude mixture was diluted in MeOH and purified by preparative HPLC-MS (method a). The purified fractions were freeze-dried and lyophilized to provide the title compound. MS (UPLC-MS): 362.2 [M+H]+, 723.5 [2M+H]+, 360.2 [M−H]−, 721.5 [2M−H]−. Rt (HPLC, method D): 4.25 min. ¹H NMR (400 MHz, DMSO-d₆) δ ppm 8.17 (s, 1H), 7.99 (s, 1H), 7.69 (d, J=7.75 Hz, 1H), 7.67 (d, J=7.65 Hz, 1H), 7.47-7.38 (m, 3H), 7.30 (d, J=7.45 Hz, 1H), 7.15 (d, J=7.35 Hz, 1H), 7.10 (t, J=7.45 Hz, 1H), 6.93 (d, J=8.05 Hz, 1H), 6.82 (d, J=7.25 Hz, 1H), 5.23 (s, 2H), 3.99 (s, 2H), 3.45 (s, 2H), 2.46 (s, 3H).

### Example 289

### 2-(2-((3′-(aminomethyl)-6-cyano-[1,1′-biphenyl]-3-yl)methoxy)phenyl)acetic acid

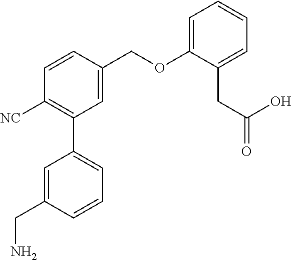 [SMILES: N#Cc1ccc(COc2ccccc2CC(=O)O)cc1-c1cccc(CN)c1]

To a solution of tert-butyl 2-(2-((3′-(((tert-butoxycarbonyl)amino)methyl)-6-cyano-[1,1′-biphenyl]-3-yl)methoxy)phenyl)acetate (Intermediate 165) (110 mg, 0.198 mmol) in dioxane (2 mL) was added HCl (4N in dioxane, 0.99 mL, 3.95 mmol) and the reaction mixture was stirred at RT for 48 hours. The solvent was evaporated and the crude mixture purified by preparative HPLC-Mass (method a). The purified fractions were freeze-dried and lyophilized to provide the title compound. MS (UPLC-MS): 373.2 [M+H]+, 745.5 [2M+H]+, 371.2 [M−H]−, 743.4 [2M−H]−. Rt (HPLC, method D): 2.99 min. ¹H NMR (400 MHz, DMSO-d₆) δ ppm 8.02 (s, 1H), 7.9 (m, 2H), 7.74 (d, J=7.85 Hz, 1H), 7.62 (d, J=8.65 Hz, 1H), 7.51 (t, J=7.70 Hz, 1H), 7.44 (d, J=7.70 Hz, 1H), 7.10 (m, 2H), 6.94 (d, J=8.4 Hz, 1H), 6.93 (t, J=7.35 Hz, 1H), 5.31 (s, 2H), 4.00 (s, 2H), 1.90 (s, 2H).

### Example 290

### 2-(2-((3′-(aminomethyl)-[1,1′-biphenyl]-3-yl)methoxy)phenyl)propanoic acid

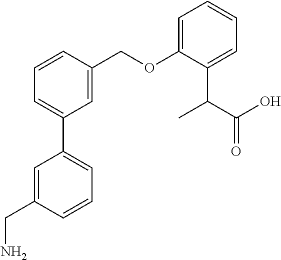 [SMILES: CC(C(=O)O)c1ccccc1OCc1cccc(-c2cccc(CN)c2)c1]

To a solution of tert-butyl 2-(2-((3′-(((tert-butoxycarbonyl)amino)methyl)-[1,1′-biphenyl]-3-yl)methoxy)phenyl)propanoate (Intermediate 167) (82 mg, 0.16 mmol) in dioxane (2.7 mL) was added HCl (4N in dioxane, 1.18 mL, 4.73 mmol). The reaction mixture was stirred for 18 hours, then was concentrated and the crude mixture was purified by preparative HPLC-MS (method C). MS (UPLC-MS): 362.3 [M+H]+, 360.3 [M−H]−. Rₜ (HPLC, method b): 3.36 min. ¹H NMR (400 MHz, DMSO-d₆+1 drop of TFA) δ ppm 8.22 (br. s., 3H), 7.83 (m, 2H), 7.75 (d, J=7.85 Hz, 1H), 7.66 (d, J=7.4 Hz, 1H), 7.56-7.46 (m, 4H), 7.25 (m, 2H), 7.10 (d, J=8.25 Hz, 1H), 6.96 (t, J=7.55 Hz, 1H), 5.23 (s, 2H), 4.14 (m, 2H), 4.01 (m, 1H), 1.38 (d, J=7.15 Hz, 3H).

### Example 291

### 2-(2-((3′-(aminomethyl)-5′-chloro-[1,1′-biphenyl]-3-yl)methoxy)phenyl)acetic acid

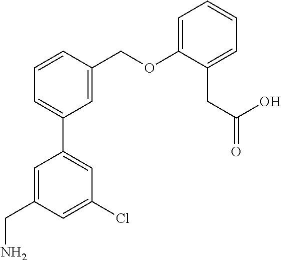 [SMILES: NCc1cc(Cl)cc(-c2cccc(COc3ccccc3CC(=O)O)c2)c1]

The title compound was synthesized in the same manner as Example 277. MS (UPLC-MS): 382.2 [M+H]+, 763.3 [2M+H]+, 380.2 [M−H]−, 761.3 [2M−H]−. Rt (HPLC, method b): 3.48 min. ¹H NMR (400 MHz, DMSO-d₆) δ ppm 8.22 (s, 1H), 8.04 (s, 1H), 7.75 (s, 1H), 7.72 (d, J=7.6 Hz, 1H), 7.49-7.41 (m, 3H), 7.13-7.08 (m, 2H), 7.93 (d, J=8.05 Hz, 1H), 6.83 (t, J=7.45 Hz, 1H), 5.23 (s, 2H), 3.98 (s, 2H), 3.42 (s, 2H).

### Example 292

### Example 292-A

### tert-butyl 2-(2-((3′-(aminomethyl)-5′-methyl-[1,1′-biphenyl]-3-yl)methoxy)phenyl)acetate

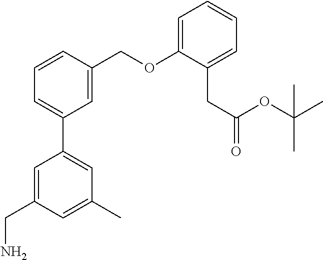 [SMILES: Cc1cc(CN)cc(-c2cccc(COc3ccccc3CC(=O)OC(C)(C)C)c2)c1]

The title compound was prepared using the same protocol as described for the preparation of methyl 2-(2-((3′-(((tert-butoxycarbonyl)(methyl)amino)methyl)-[1,1′-biphenyl]-3-yl)methoxy)phenyl)acetate using tert-Butyl 2-(2-((3-(4,4,5,5-tetramethyl-1,3,2-dioxaborolan-2-yl)benzyl)oxy)phenyl)acetate (60 mg, 0.141 mmol), 1-(3-bromo-5-methylphenyl)methanamine hydrochloride)(Anichem NC 2184) (66.9 mg, 0.283 mmol), K₃PO₄ (2N in water, 0.283 mL, 0.566 mmol) and PdCl₂(dppf).CH₂Cl₂ adduct (7.77 mg, 7.07 μmol) in acetonitrile (3 mL). The crude mixture was purified by flash column chromatography on silica gel (0-10% MeOH/CH₂Cl₂) to give the title compound. TLC, R_{f}(c-hexane/EtOAc 4:1): 0.3. MS (UPLC-MS): 418.3 [M+H]+, 935.5 [2M+H]+, 416.4 [M−H]+, 462.2 [M+HCOO]−. Rt (HPLC, method E): 2.20 min.

### Example 292-B

### 2-(2-((3′-(aminomethyl)-5′-methyl-[1,1′-biphenyl]-3-yl)methoxy)phenyl)acetic acid

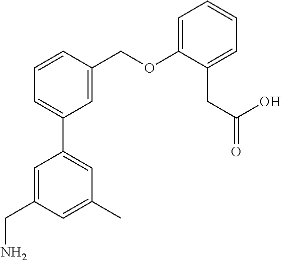 [SMILES: Cc1cc(CN)cc(-c2cccc(COc3ccccc3CC(=O)O)c2)c1]

The title compound was prepared using the same protocol as described for the preparation of Example 291 using tert-butyl 2-(2-((3′-(aminomethyl)-5′-methyl-[1,1′-biphenyl]-3-yl)methoxy)phenyl)acetate. MS (UPLC-MS): 362.2 [M+H]+, 723.3 [2M+H]+, 360.1 [M−H]−, 721.4 [2M−H]−. Rₜ (HPLC, method D): 3.37 min. ¹H NMR (400 MHz, DMSO-d₆) δ ppm 8.06 (s, 1H), 8.03 (s, 1H), 7.65 (d, J=7.65 Hz, 1H), 7.51 (s, 1H), 7.44 (t, J=7.75 Hz, 1H), 7.37 (d, J=7.55 Hz, 1H), 7.12-7.08 (m, 3H), 6.93 (d, J=8.1 Hz, 1H), 6.82 (t, J=7.45 Hz, 1H), 5.22 (s, 2H), 3.92 (s, 2H), 3.41 (s, 2H), 2.37 (s, 3H).

### Example 293

### 2-(2-((3′-(aminomethyl)-[1,1′-biphenyl]-3-yl)methoxy)-4-ethylphenyl)acetic acid

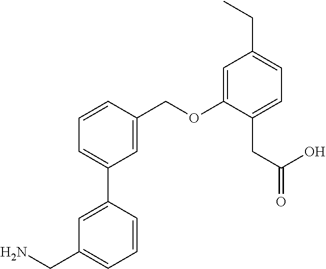 [SMILES: CCc1ccc(CC(=O)O)c(OCc2cccc(-c3cccc(CN)c3)c2)c1]

To a solution of 2-(2-((3′-(((tert-butoxycarbonyl)amino)methyl)-[1,1′-biphenyl]-3-yl)methoxy)-4-ethylphenyl)acetic acid (Intermediate 171) (44 mg, 0.093 mmol) in dioxane (1.2 mL) was added HCl (4N in dioxane, 0.46 mL, 1.85 mmol). The reaction mixture was stirred at RT until completion, then was concentrated and the crude mixture was purified by preparative HPLC-MS (method A). MS (UPLC-MS): 376.2 [M+H]+, 751.4 [2M+H]+, 374.2 [M−H]−, 749.4 [2M−H]−. Rt (HPLC, method D): 3.74 min. ¹H NMR (400 MHz, DMSO-d₆) δ ppm 8.29 (s, 1H), 8.06 (s, 1H), 7.68 (m, 2H), 7.47-7.37 (m, 3H), 7.31 (d, J=7.6 Hz, 1H), 7.00 (d, J=7.5 Hz, 1H), 6.81 (s, 1H), 6.67 (d, J=7.6 Hz, 1H), 5.21 (s, 2H), 3.98 (s, 2H), 3.36 (s, 2H), 2.56 (q, J=7.6 Hz, 2H, overlap with DMSO signal), 1.16 (t, J=7.6 Hz, 3H).

### Example 294

The compounds in the table below were synthesized as described in Example 293 using Intermediates 172-179.

| | | | MS (UPLC-MS). |
|---|---|---|---|
| | | | HPLC, Rₜ |
| Ex. | Structure/Chemical Name | ¹H NMR | (method) |
| 294-A | 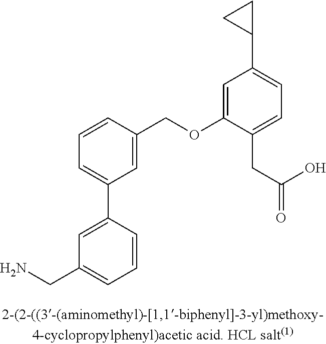 [SMILES: NCc1cccc(-c2cccc(COc3cc(C4CC4)ccc3CC(=O)O)c2)c1] | (400 MHz, DMSO-d₆) δ ppm 7.85 (s, 1H )7.80 (s, 1H) 7.74 (d, J = 7.55 Hz, 1H) 7.66 (d, J = 7.45 Hz, 1H) 7.57-7.46 (m, 4H) 7.08 (d, J = 7.65 Hz, 1H) 6.77 (s, 1H) 6.64 (d, J = 7.6 Hz, 1H) 5.20 (s, 2H) 4.13 (s, 2H) 3.54 (s, 2H) 1.89 (m, 1H) 0.93 (m, 2H), 0.67 (m, 2H) | 388.2 [M + H]+, 775.4 [2M + H]+, 386.2 [M − H]−, 773.4 [2M − H]−. 3.73 min (D) |
| 294-B | 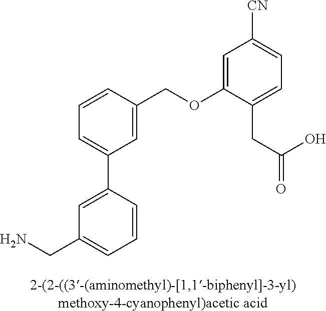 [SMILES: N#Cc1ccc(CC(=O)O)c(OCc2cccc(-c3cccc(CN)c3)c2)c1] | (400 MHz, DMSO-d₆) δ ppm 8.28 (s, 1H) 8.00 (s, 1H) 7.70 (m, 2H) 7.50-7.39 (m, 4H) 7.32 (m, 3H) 5.32 (s, 2H) 4.00 (s, 2H) 3.48 (s, 2H). | 373.2 [M + H]+, 745.3 [2M + H]+, 371.2 [M − H]−, 743.3 [2M − H]−. 3.01 min (D) |
| 294-C | 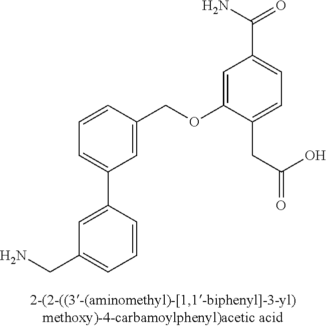 [SMILES: NCc1cccc(-c2cccc(COc3cc(C(N)=O)ccc3CC(=O)O)c2)c1] | NMR (400 MHz, DMSO-d₆) δ ppm 8.28 (s, 1H) 8.06 (s, 1H) 7.89 (s, 1H, NH) 7.69 (m, 2H) 7.49-7.37 (m, 5H) 7.31 (d, J = 7.55 Hz, 1H) 7.26 (s, 1H, NH) 7.17 (d, J = 7.6 Hz, 1H) 5.28 (s, 2H) 3.99 (s, 2H) 3.45 (s, 2H). | 391.2 [M + H]+. 781.2 [2M + H]+, 389.2 [M − H]−, 779.3 [2M − H]−. 2.23 min (D) |
| 294-D | 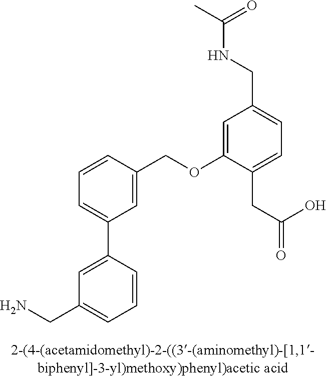 [SMILES: CC(=O)NCc1ccc(CC(=O)O)c(OCc2cccc(-c3cccc(CN)c3)c2)c1] | (400 MHz, DMSO-d₆) δ ppm 8.28 (br. s., 2H) 7.93-8.05 (s, 1H) 7.68 (t, J = 8.6 Hz, 1H) 7.45-7.29 (m, 4H) 7.05 (d, J = 7.50 Hz, 1H) 6.86 (s, 1H) 6.71 (d, J = 7.5 Hz, 1H) 5.19 (s, 2H) 4.18 (d, J = 5.85 Hz, 2H) 3.98 (s, 2H) 3.38 (s, 2H) 1.85 (s, 3H). | 419.3 [M + H]+, 837.4 [2M + H]+, 417.3 [M − H]−, 463.2 [M + HCOO]−, 835.5 [2M − H]−. 2.39 min (D) |
| 294-E | 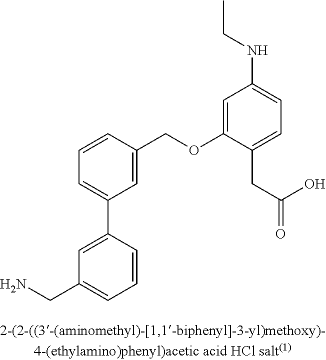 [SMILES: CCNc1ccc(CC(=O)O)c(OCc2cccc(-c3cccc(CN)c3)c2)c1] | (400 MHz, DMSO-d₆) δ ppm 8.34 (br. s., 3H) 7.88 (s, 1H) 7.81 (s, 1H) 7.74 (d, J = 7.70 Hz, 1H) 7.67 (d, J = 7.40 Hz, 1H) 7.56-7.48 (m, 4H) 7.19 (m, 1H) 5.20 (s, 2H) 4.13 (q, J = 7.10 Hz, 2H) 3.56 (s, 2H) 3.22 (br. s., 2H) 1.18 (t, J = 7.10 Hz 3H). | 391.2 [M + H]+, 389.2 [M − H]−, 781.4 [2M + H]+, 779.4 [2M − H]−. 2.02 min (D) |
| 294-F | 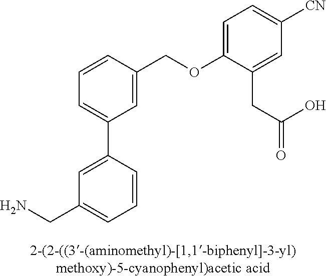 [SMILES: N#Cc1ccc(OCc2cccc(-c3cccc(CN)c3)c2)c(CC(=O)O)c1] | NMR (400 MHz, DMSO-d₆) δ ppm 8.27 (s, 1H) 7.97 (s, 1H) 7.71-7.67 (m, 2H) 7.60 (d, J = 8.60 Hz, 1H) 7.55 (s, 1H), 7.49-7.39 (m, 3H) 7.32 (d, J = 7.55 Hz, 1H) 7.11 (d, J = 8.50 Hz, 1H) 5.35 (s, 2H) 4.01 (s, 2H) 3.44 (s, 2H). | 373.2 [M + H]+, 745.3 [2M + H]+ 371.2 [M − H]−, 743.3 [2M − H]−. 2.95 min (D) |
| 294-G | 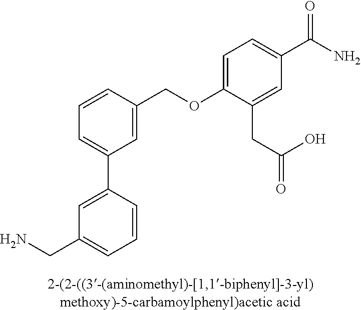 [SMILES: NCc1cccc(-c2cccc(COc3ccc(C(N)=O)cc3CC(=O)O)c2)c1] | NMR (400 MHz, DMSO-d₆) δ ppm 8.27 (s, 1H) 8.02 (s, 1H) 7.76 (br. s., 1H) 7.70-7.64 (m, 4H) 7.48-7.39 (m, 3H) 7.32 (d, J = 7.55 Hz, 1H) 7.09 (br. s., 1H) 6.96 (d, J = 8.50 Hz, 1H) 5.31 (s, 2H) 4.00 (s, 2H). | 391.2 [M + H]+, 781.3 [2M + H]+ 389.2 [M − H]−, 435.2 [M + HCOO]−, 779.3 [2M − H]−. 2.27 min (D) |

⁽¹⁾In the second Boc deprotection step, the crude mixture was triturated in CH₂Cl₂ and the precipitate was filtered to give the title compound as a HCl salt.

### Example 295

### Example 295-A

### methyl 2-(2-((3′-(((tert-butoxycarbonyl)amino)methyl)-[1,1′-biphenyl]-3-yl)meth oxy)-4-(cyclopropylmethyl)phenyl)acetate

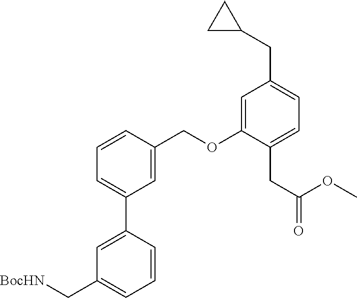 [SMILES: COC(=O)Cc1ccc(CC2CC2)cc1OCc1cccc(-c2cccc(CNC(=O)OC(C)(C)C)c2)c1]

To a mixture of methyl 2-(4-bromo-2-((3′-(((tert-butoxycarbonyl)amino)methyl)-[1,1′-biphenyl]-3-yl)methoxy)phenyl)acetate (80 mg, 0.148 mmol), potassium cyclopropylmethyl trifluoroborate (28.8 mg, 0.178 mmol), Pd(OAc)₂ (2 mg, 8.9 μmol), Ruphos (8.29 mg, 0.018 mmol) and K₂CO₃ (61.4 mg, 0.444 mmol) under argon atmosphere were added toluene (0.750 mL) and water (0.075 mL). The reaction mixture was stirred and heated at 80° C. for 17 hours. To allow completion the reaction mixture was worked-up using the procedure described below and restarted by adding the same quantities of potassium cyclopropylmethyl trifluoroborate, Pd(OAc)₂, K₂CO₃ and solvents and stirring for 21 hours. The reaction mixture was allowed to cool to RT and was filtered through a pad of Celite. Water and EtOAc were added, the layers were separated and the aqueous layer was extracted with EtOAc (×3). The combined organic extracts were dried (phase separator) and concentrated. The crude residue was treated with a metal scavenger using the same protocol as described in the preparation of methyl 2-(2-((3′-(((tert-butoxycarbonyl)(methyl)amino)methyl)-[1,1′-biphenyl]-3-yl)methoxy)phenyl)acetate and purified by flash chromatography on silica gel (0-20% AcOEt/c-hexane) to give the title compound contaminated with methyl 2-(4-(but-3-en-1-yl)-2-((3′-(((tert-butoxycarbonyl)amino)methyl)-[1,1′-biphenyl]-3-yl)methoxy)phenyl)acetate. TLC, R_{f} (c-hexane/EtOAc 7:3): 0.7. MS (UPLC-MS): 516.3 [M+H]+, 533.3 [M+18]+, 514.5 [M−H]−, 560.3 [M+HCOO]−. Rₜ (HPLC, method E): 2.97 min.

### Example 295-B

### 2-(2-((3′-(((tert-butoxycarbonyl)amino)methyl)-[1,1′-biphenyl]-3-yl)methoxy)-4-(cyclopropylmethyl)phenyl)acetic acid

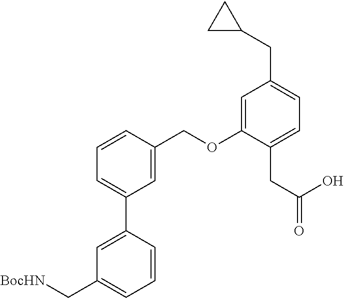 [SMILES: CC(C)(C)OC(=O)NCc1cccc(-c2cccc(COc3cc(CC4CC4)ccc3CC(=O)O)c2)c1]

To a mixture of methyl 2-(2-((3′-(((tert-butoxycarbonyl)amino)methyl)-[1,1′-biphenyl]-3-yl)methoxy)-4-(cyclopropylmethyl)phenyl)acetate contaminated with methyl 2-(4-(but-3-en-1-yl)-2-((3′-(((tert-butoxycarbonyl)amino)methyl)-[1,1′-biphenyl]-3-yl)methoxy)phenyl)acetate (22 mg, 0.043 mmol) in MeOH (0.4 mL) and water (40 μL) NaOH (1N in water, 213 μL, 0.213 mmol) was added and the reaction was stirred at RT for 16 hours. NaOH (1N in water, 213 μL, 0.213 mmol) was added again and the mixture further stirred until completion. The reaction mixture was acidified with 1N aq. HCl, CH₂Cl₂ and water were added, the layers were separated and the aqueous layer was extracted with CH₂Cl₂ (×3). The combined organic extracts were dried (phase separator) and concentrated to give the title compound contaminated with 2-(4-(but-3-en-1-yl)-2-((3′-(((tert-butoxycarbonyl)amino)methyl)-[1,1′-biphenyl]-3-yl)methoxy)phenyl)acetic acid. MS (UPLC-MS): 500.3 [M−H]−, 1001.5 [2M−H]−, 502.3 [M+H]+, 519.3 [M+18]+, 1020.6 [2M+18]+. Rₜ (HPLC, method D): 6.20 min. To a mixture of 2-(2-((3′-(((tert-butoxycarbonyl)amino)methyl)-[1,1′-biphenyl]-3-yl)methoxy)-4-(cyclopropylmethyl)phenyl)acetic acid contaminated with methyl 2-(4-(but-3-en-1-yl)-2-((3′-(((tert-butoxycarbonyl)amino)methyl)-[1,1′-biphenyl]-3-yl)methoxy)phenyl)acetate (25 mg, 0.050 mmol) and 4-methylmorpholine N-oxide monohydrate (13.47 mg, 0.10 mmol) in acetone (970 μL) and water (162 μL) was added osmium tetroxide (4% in water, 31.7 μL, 4.98 μmol). The reaction mixture was stirred at RT for 22 hours to allow complete conversion of methyl 2-(4-(but-3-en-1-yl)-2-((3′-(((tert-butoxycarbonyl)amino)methyl)-[1,1′-biphenyl]-3-yl)methoxy)phenyl)acetate into the corresponding diol. A solution of 1N aqueous sodium thiosulfate (3 mL) was added and the mixture was stirred for 2 hours. Water and CH₂Cl₂ were added, the layers were separated and the aqueous layer was extracted with CH₂Cl₂ (2×). The combined organic extracts were dried (phase separator) and concentrated to give the title compound contaminated with 2-(2-((3′-(((tert-butoxycarbonyl)amino)methyl)-[1,1′-biphenyl]-3-yl)methoxy)-4-(3,4-dihydroxybutyl)phenyl)acetic acid. MS (UPLC-MS, method d): Rt: 1.47 min 502.2 [M+H]+, 1020.6 [2M+18]+, 500.2 [M−H]−, 1001.5 [2M−H]−; Rt: 1.08 min: 536.2 [M+H]+, 553.3 [M+18]+, 1088.5 [2M+18]+, 534.2 [M−H]−, 1069.6 [2M−H]−.

### Example 295-C

### 2-(2-((3′-(aminomethyl)-[1,1′-biphenyl]-3-yl)methoxy)-4-(cyclopropylmethyl)phenyl)acetic acid

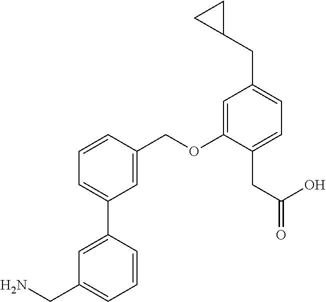 [SMILES: NCc1cccc(-c2cccc(COc3cc(CC4CC4)ccc3CC(=O)O)c2)c1]

To a solution of 2-(2-((3′-(((tert-butoxycarbonyl)amino)methyl)-[1,1′-biphenyl]-3-yl)methoxy)-4-(cyclopropylmethyl)phenyl)acetic acid contaminated with 2-(2-((3′-(((tert-butoxycarbonyl)amino)methyl)-[1,1′-biphenyl]-3-yl)methoxy)-4-(3,4-dihydroxybutyl)phenyl)acetic acid (28 mg, 0.056 mmol) in dioxane (0.7 mL) was added HCl (4N in dioxane, 0.42 mL, 1.675 mmol). The reaction mixture was stirred at RT for 22 hours, then was concentrated and the crude mixture was purified by preparative HPLC-MS (method A). The purified fractions were freeze-dried and lyophilized to give a white powder. CH₃CN and aq. HCl (0.5N, 0.3 mL) were added, the solution was stirred at RT for 15 min, then freeze-dried and lyophilized to give the title compound as HCL salt. MS (UPLC-MS): 402.3 [M+H]+, 803.4 [2M+H]+, 400.3 [M−H]−, 446.2 [M+HCOO]−, 801.5 [2M−H]−. Rt (HPLC, method D): 4.03 min. ¹H NMR (400 MHz, DMSO-d₆) δ ppm 8.07 (br. s., 3H), 7.68 (s, 1H), 7.64 (s, 1H), 7.56 (d, J=7.75 Hz, 1H), 7.48 (d, J=7.35 Hz, 1H), 7.39-7.28 (m, 4H), 6.95 (d, J=7.50 Hz, 1H), 6.81 (s, 1H), 6.63 (d, J=7.55 Hz, 1H), 5.03 (s, 2H), 3.95 (s, 2H), 3.38 (s, 2H), 2.31 (m, 2H, overlap with DMSO signal), 0.78 (m, 1H), 0.26 (m, 2H), 0.01 (m, 2H).

### Example 296

### 2-acetamido-2-(2-((3′-(aminomethyl)-[1,1′-biphenyl]-3-yl)methoxy)phenyl)acetic acid

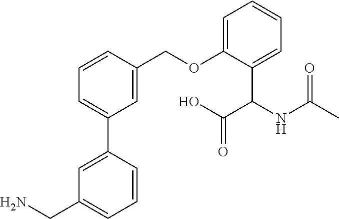 [SMILES: CC(=O)NC(C(=O)O)c1ccccc1OCc1cccc(-c2cccc(CN)c2)c1]

To a solution of 2-acetamido-2-(2-((3′-(((tert-butoxycarbonyl)amino)methyl)-[1,1′-biphenyl]-3-yl)methoxy)phenyl)acetic acid (Intermediate 183) (0.07 mmol) in dioxane (1 mL) was added HCl (4N in dioxane, 0.52 mL, 2.08 mmol). The reaction mixture was stirred at RT for 14 hours, then was concentrated and the crude mixture was purified by preparative HPLC-MS (method A).

The purified fractions were freeze-dried and lyophilized to give the title compound as a mixture of 2 enantiomers (73% ee, determined by analytical HPLC: Chiralcel OZ-I 20 um, 250×4.0 mm, mobile phase: Hept/CH₂Cl₂/EtOH/TFA 70:25:5:0.1, Flow rate: 0.7 ml/min, UV detection 254 nM, Rₜ (major enantiomer): 14.15 min, Rₜ (minor enantiomer): 20.90 min). MS (UPLC-MS): 405.2 [M+H]+, 403.2 [M−H]−, 807.3 [2M−H]−. Rt (HPLC, method b): 2.69 min. ¹H NMR (400 MHz, DMSO-d₆) δ ppm 8.30 (m, 2H), 7.99 (d, J=7.75 Hz, 1H), 7.71-7.66 (m, 2H), 7.48-7.39 (m, 3H), 7.28 (m, 2H), 7.02 (t, J=7.75 Hz, 1H), 6.79 (m, 2H), 5.64 (d, J=7.70 Hz, 1H), 5.35 (s, 2H), 4.04 (d, J=13.7 Hz, 1H), 3.94 (d, J=13.7 Hz, 1H), 1.86 (s, 3H).

### Example 297-A

### Methyl 2-(2-((3-chlorobenzyl)oxy)phenyl)-2-(2-phenylcyclopropanecarboxamido)acetate

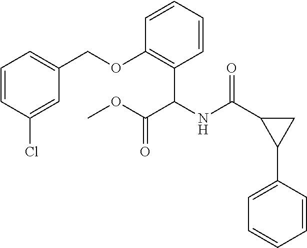 [SMILES: COC(=O)C(NC(=O)C1CC1c1ccccc1)c1ccccc1OCc1cccc(Cl)c1]

Methyl 2-(2-((3-chlorobenzyl)oxy)phenyl)-2-(2-phenylcyclopropanecarboxamido)acetate was prepared following the same procedure as for Intermediate 180 using racemic trans-2 phenyl-1-cyclopropane carboxylic acid. The two resulting diastereomers were separated by flash chromatography (0-30% AcOEt/c-hexane).

### Diastereomer 1: MS (ESI+) m/z 450.2/452.2 (M+H); TLC R_{f} (Cyclohexane/EtOAc 8:2)=0.29.

### Diastereomer 2: MS (ESI+) m/z 450.2/452.2 (M+H); TLC R_{f} (Cyclohexane/EtOAc 8:2)=0.25.

### Example 297-B

### 2-(2-((3′-(aminomethyl)-[1,1′-biphenyl]-3-yl)methoxy)phenyl)-2-(2-phenylcyclopropanecarboxamido)acetic acid

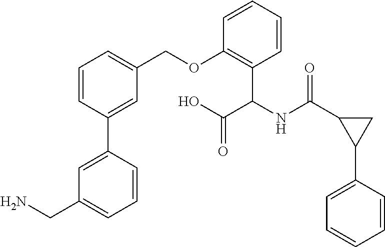 [SMILES: NCc1cccc(-c2cccc(COc3ccccc3C(NC(=O)C3CC3c3ccccc3)C(=O)O)c2)c1]

The title compound was prepared in analogy to Example 296 and preceding steps using Methyl 2-(2-((3-chlorobenzyl)oxy)phenyl)-2-(2-phenylcyclopropanecarboxamido)acetate (Diastereomer 1). MS (ESI+) m/z 507.3 (M+H). ¹H NMR (400 MHz, DMSO-d₆) δ 12.75 (s, 1H), 8.94-8.78 (m, 1H), 8.23 (s, 3H), 7.86 (d, J=4.7 Hz, 2H), 7.81-7.60 (m, 2H), 7.60-7.43 (m, 4H), 7.41-7.09 (m, 8H), 7.09-6.93 (m, 1H), 5.94 (dd, J=8.1, 6.6 Hz, 1H), 5.27 (s, 2H), 4.15 (s, 2H), 2.34-2.10 (m, 2H), 1.36-1.06 (m, 2H).

Compounds of invention are active on factor D inhibition. Data on Table 1 collected using the assay of Biological Example 2.

TABLE 1

| Example Number | IC₅₀ (μM) |
|---|---|
| Example 1-C | 13.43 |
| Example 2-B | 0.12 |
| Example 3-B | 80.19 |
| Example 4-A | 33.08 |
| Example 4-B | 4.60 |
| Example 5 | 0.045 |
| Example 6-B | 0.106 |
| Example 7-D | 0.244 |
| Example 8-A | 5.05 |
| Example 8-B | 0.353 |
| Example 9 | 0.071 |
| Example 10-B | 0.396 |
| Example 11-D | 0.017 |
| Example 12 | 0.08 |
| Example 13-B | 0.009 |
| Example 14-A | 0.015 |
| Example 14-B | 0.011 |
| Example 14-C | 0.005 |
| Example 14-D | 0.021 |
| Example 14-E | 0.01 |
| Example 15-B | 0.009 |
| Example 16-C | 0.338 |
| Example 16-D | 0.47 |
| Example 17-B | 0.399 |
| Example 18-B | 8.79 |
| Example 19-B | 0.666 |
| Example 20-B | 0.016 |
| Example 21-A | 0.414 |
| Example 21-B | 0.047 |
| Example 21-C | 0.109 |
| Example 21-D | 0.989 |
| Example 21-E | 0.086 |
| Example 21-F | 3.12 |
| Example 21-G | 7.68 |
| Example 21-H | 0.94 |
| Example 21-I | 0.469 |
| Example 21-J | 0.517 |
| Example 21-K | 0.126 |
| Example 21-L | 0.105 |
| Example 21-M | 0.022 |
| Example 21-N | 0.138 |
| Example 21-O | 0.012 |
| Example 21-P | 0.118 |
| Example 21-Q | 0.022 |
| Example 22-C | 0.835 |
| Example 23-C | 0.396 |
| Example 24 | 1.74 |
| Example 25-D | 0.011 |
| Example 26-B | 0.094 |
| Example 27 | 0.05 |
| Example 28-B | 0.057 |
| Example 29 | 0.062 |
| Example 30-D | 0.031 |
| Example 31-B | 0.016 |
| Example 32 | 0.049 |
| Example 33 | 0.007 |
| Example 34-C | 0.005 |
| Example 35-B | 0.013 |
| Example 36-C | 0.036 |
| Example 37 | 0.041 |
| Example 38 | 14.30 |
| Example 39 | 40.49 |
| Example 40 | 12.793 |
| Example 41 | 0.074 |
| Example 42 | 0.013 |
| Example 43 | 1.13 |
| Example 44 | 0.009 |
| Example 45 | 0.012 |
| Example 46 | 0.006 |
| Example 47 | 1.93 |
| Example 48 | 2.89 |
| Example 49 | 2.27 |
| Example 50 | 0.002 |
| Example 51 | 0.006 |
| Example 52 | 0.007 |
| Example 53 | 0.003 |
| Example 54 | 0.003 |
| Example 55 | 0.013 |
| Example 56-A | 0.107 |
| Example 56-B | 0.117 |
| Example 57 | 0.026 |
| Example 58-D | 0.271 |
| Example 59-C | 0.095 |
| Example 60 | 0.047 |
| Example 61-B | 0.032 |
| Example 62 | 0.054 |
| Example 63 | 0.016 |
| Example 64 | 0.063 |
| Example 65 | 0.017 |
| Example 66 | 0.033 |
| Example 67-B | 0.03 |
| Example 67-C | 2.93 |
| Example 68 | 0.02 |
| Example 69 | 0.026 |
| Example 70 | 0.011 |
| Example 71 | 0.009 |
| Example 72-B | 0.058 |
| Example 73-C | 0.022 |
| Example 74-D | 0.009 |
| Example 75-B | 0.026 |
| Example 76 | 0.006 |
| Example 77-B | 0.055 |
| Example 78-B | 0.022 |
| Example 79-A | 0.005 |
| Example 79-B | 0.009 |
| Example 79-C | 0.022 |
| Example 79-D | 0.014 |
| Example 79-E | 0.012 |
| Example 79-F | 0.006 |
| Example 79-G | 0.022 |
| Example 79-H | 0.017 |
| Example 79-I | 0.019 |
| Example 80-C | 0.143 |
| Example 81 | 0.02 |
| Example 82-B | 0.019 |
| Example 83-D | 0.022 |
| Example 84 | 0.005 |
| Example 85 | 0.709 |
| Example 86 | 1.26 |
| Example 87 | 0.814 |
| Example 88 | 0.958 |
| Example 89 | 5.251 |
| Example 90 | 0.823 |
| Example 91 | 22.25 |
| Example 92 | 8.47 |
| Example 93-B | 6.719 |
| Example 94-C | 0.027 |
| Example 95-B | 2.24 |
| Example 96-D | 0.021 |
| Example 97 | 0.045 |
| Example 98 | 0.004 |
| Example 99-C | 1.85 |
| Example 100 | 2.17 |
| Example 101-E | 0.008 |
| Example 102-B | 0.009 |
| Example 103-B | 0.01 |
| Example 104 | 0.006 |
| Example 105-C | 0.015 |
| Example 106 | 0.005 |
| Example 107-C | 0.002 |
| Example 108-B | 0.007 |
| Example 109-B | 0.022 |
| Example 110-C | 0.03 |
| Example 111-B | 0.003 |
| Example 112-B | 0.007 |
| Example 113-B | 0.03 |
| Example 114-B | 0.006 |
| Example 115-A | 0.003 |
| Example 115-B | 0.008 |
| Example 115-C | 0.005 |
| Example 116-A | 0.021 |
| Example 116-B | 0.004 |
| Example 116-C | 0.004 |
| Example 116-D | 0.009 |
| Example 116-E a) | 0.009 |
| Example 116-E b) (tr = 3.8 min) | 0.011 |
| Example 116-E b) (tr = 5.7 min) | 0.013 |
| Example 116-F | 0.005 |
| Example 116-G a) | 0.006 |
| Example 116-G b) (tr = 2.1 min) | 0.011 |
| Example 116-G b) (tr = 3.4 min) | 0.009 |
| Example 120-F | 0.008 |
| Example 216-Y | 0.019 |
| Example 216-Z | 0.013 |
| Example 216-AA | 0.032 |
| Example 216-AB | 0.055 |
| Example 216-AC | 0.033 |
| Example 216-AD | 0.02 |
| Example 216-AE | 0.053 |
| Example 216-AF | 0.016 |
| Example 216-AG | 0.024 |
| Example 216-AH | 0.09 |
| Example 216-AI | 0.024 |
| Example 216-AJ | 0.46 |
| Example 217-D | 0.063 |
| Example 218-G | 0.006 |
| Example 218-I | 0.01 |
| Example 219-D | 0.061 |
| Example 220-D | 0.044 |
| Example 221 a) | 0.006 |
| Example 221b) | 0.004 |
| Example 222 | 0.016 |
| Example 223-B | 0.009 |
| Example 224-B | 0.009 |
| Example 225-C | 0.016 |
| Example 226-C | 0.008 |
| Example 227 | 0.01 |
| Example 229 | 0.107 |
| Example 230 | 0.003 |
| Example 231-B | 0.011 |
| Example 231-F | 0.009 |
| Example 232 | 0.002 |
| Example 233 | 0.004 |
| Example 234-B | 0.004 |
| Example 235 | 0.034 |
| Example 236-C | 0.009 |
| Example 237 | 0.011 |
| Example 238 | 0.023 |
| Example 239-C | 0.013 |
| Example 240-B | 0.11 |
| Example 241-C | 0.04 |
| Example 242-C | 0.09 |
| Example 243-D | 0.013 |
| Example 244 | 0.003 |
| Example 245 | 0.009 |
| Example 246-C | <0.001 |
| Example 247 | 0.010 |
| Example 248 | 0.14 |
| Example 249 | 26.9 |
| Example 250-D | 0.113 |
| Example 251 | 0.158 |
| Example 252 | 1.58 |
| Example 253-E | 0.013 |
| Example 254 | 0.032 |
| Example 255 | 0.009 |
| Example 256-B | 0.003 |
| Example 257 | 0.009 |
| Example 258-C | 0.011 |
| Example 259-E | 0.022 |
| Example 260 | 0.039 |
| Example 261 | 0.024 |
| Example 262 | 0.041 |
| Example 263 | 0.014 |
| Example 264 | 0.015 |
| Example 265 | 0.007 |
| Example 266 | 0.034 |
| Example 267 | 0.039 |
| Example 268-C | 54.8 |
| Example 269-E | 0.009 |
| Example 270 | 0.011 |
| Example 271 | 0.01 |
| Example 272 | 0.01 |
| Example 273-B | 0.014 |
| Example 274 | 0.34 |
| Example 275 | 0.013 |
| Example 276 | 0.16 |
| Example 277-C | 0.19 |
| Example 278-B | 0.49 |
| Example 279-B | 0.29 |
| Example 280 | 0.006 |
| Example 281-E | 0.014 |
| Example 282-D | 0.016 |
| Example 283 | 0.013 |
| Example 284-B | 0.228 |
| Example 285-A | 0.035 |
| Example 285-B | 0.058 |
| Example 286-E | 0.006 |
| Example 287-G | 0.037 |
| Example 116-H a) | 0.001 |
| Example 116-H b) (tr = 3.0 min) | 0.01 |
| Example 116-H b) (tr = 4.6 min) | 0.015 |
| Example 116-I a) | 0.013 |
| Example 116-I b) (tr = 4.0 min) | 0.026 |
| Example 116-I b) (tr = 6.3 min) | 0.024 |
| Example 116-J | 0.005 |
| Example 116-K | 0.108 |
| Example 116-L | 10.31 |
| Example 116-M | 1.23 |
| Example 116-N | 52.53 |
| Example 117-B | 0.083 |
| Example 118 | 0.067 |
| Example 119-B | 0.013 |
| Example 120-A | 0.015 |
| Example 120-B | 0.055 |
| Example 120-C | 0.003 |
| Example 120-D | 0.093 |
| Example 120-E | 0.017 |
| Example 121-C | 0.38 |
| Example 122 | 0.015 |
| Example 123 | 0.312 |
| Example 124 | 0.099 |
| Example 125 | 4.452 |
| Example 126-B | 0.003 |
| Example 127-B | 0.062 |
| Example 128-C | 0.003 |
| Example 129 | 0.009 |
| Example 130 | 0.015 |
| Example 131 | 0.018 |
| Example 132 | 0.018 |
| Example 133 | 0.009 |
| Example 134 | 0.013 |
| Example 135 | 0.005 |
| Example 136 | 0.005 |
| Example 137 | 0.021 |
| Example 138-A | 0.002 |
| Example 138-B | 0.088 |
| Example 138-C | 1.52 |
| Example 138-D | 0.223 |
| Example 138-E | 1.99 |
| Example 138-F | 2.19 |
| Example 138-G | 0.012 |
| Example 138-H | 0.005 |
| Example 138-I | 0.301 |
| Example 138-J | 2.87 |
| Example 138-K | 0.045 |
| Example 138-L | 0.099 |
| Example 138-M | 0.005 |
| Example 138-N | 0.028 |
| Example 139 | 0.022 |
| Example 140 | 0.003 |
| Example 141-B | 0.014 |
| Example 142-A | 0.346 |
| Example 142-B | 0.077 |
| Example 142-C | 49.60 |
| Example 142-D | 0.346 |
| Example 142-E | 0.008 |
| Example 142-F | 0.26 |
| Example 142-G | 0.012 |
| Example 142-H | 0.022 |
| Example 142-I | 0.002 |
| Example 142-J | 0.067 |
| Example 143-C | 0.132 |
| Example 144-C | 0.056 |
| Example 146-C | 18.44 |
| Example 147-B | 0.005 |
| Example 148 | 0.009 |
| Example 149 | 0.044 |
| Example 150-C | 0.004 |
| Example 151 | 0.006 |
| Example 152 | 0.011 |
| Example 153 | 0.004 |
| Example 154-C a) | 0.003 |
| Example 154-C b) | 0.004 |
| Example 155 a) | 0.003 |
| Example 155 b) | 0.004 |
| Example 156-B | 0.002 |
| Example 157 | 0.005 |
| Example 158-C | 1.787 |
| Example 159 | 0.008 |
| Example 160 | 1.135 |
| Example 161 | 0.024 |
| Example 162 | 0.002 |
| Example 163 | 0.061 |
| Example 164 | 0.013 |
| Example 165 | 0.021 |
| Example 166 | 0.023 |
| Example 167 | 0.03 |
| Example 168 | 0.027 |
| Example 169 | 0.033 |
| Example 170 | 0.022 |
| Example 171 | 2.54 |
| Example 172 | 5.88 |
| Example 173 | 0.147 |
| Example 174 | 0.111 |
| Example 175 b) (tr = 4.1 min) | 0.003 |
| Example 175 b) (tr = 9.5 min) | 0.25 |
| Example 176 | 5.74 |
| Example 177 b) (tr = 3.2 min) | 0.004 |
| Example 177 b) (tr = 8.8 min) | 0.18 |
| Example 178 | 0.002 |
| Example 179 | 0.004 |
| Example 180 | 0.149 |
| Example 181 | 0.008 |
| Example 182 | 0.008 |
| Example 183 | 0.029 |
| Example 184 | 0.436 |
| Example 185 | 0.024 |
| Example 186 | 0.014 |
| Example 187 | 0.174 |
| Example 188 | 0.017 |
| Example 189 | 1.108 |
| Example 190-A | 0.041 |
| Example 190-B (−) | 0.213 |
| Example 190-B (+) | 0.009 |
| Example 190-C | 1.21 |
| Example 190-D | 0.981 |
| Example 190-E | 0.034 |
| Example 190-F | 3.15 |
| Example 190-G | 0.122 |
| Example 190-H | 1.116 |
| Example 190-I | 0.024 |
| Example 190-J | 0.013 |
| Example 190-K | 0.133 |
| Example 191 | 20.53 |
| Example 192-B | 0.018 |
| Example 193 | 0.048 |
| Example 194 | 0.263 |
| Example 195-B | 0.107 |
| Example 196-A | 11.28 |
| Example 196-B | 0.206 |
| Example 196-C | 0.313 |
| Example 196-D | 0.642 |
| Example 196-E | 0.272 |
| Example 196-F | 0.006 |
| Example 196-G | 1.08 |
| Example 196-H | 1.18 |
| Example 196-I | 0.239 |
| Example 197 | 0.069 |
| Example 198-C | 0.199 |
| Example 199-A | 0.148 |
| Example 199-B | 0.069 |
| Example 199-C | 0.065 |
| Example 200-B | 0.022 |
| Example 201 | 0.113 |
| Example 202-B | 0.033 |
| Example 203 | 0.054 |
| Example 204-B | 0.007 |
| Example 205-B | 0.01 |
| Example 206 | 0.005 |
| Example 207-B | 0.005 |
| Example 208-A | 0.019 |
| Example 208-B | 0.027 |
| Example 208-C | 0.006 |
| Example 208-D | 0.004 |
| Example 208-E | 0.003 |
| Example 208-F | 0.009 |
| Example 208-G | 0.006 |
| Example 208-H | 0.004 |
| Example 209-A | 0.003 |
| Example 209-B | 0.004 |
| Example 120-G | 0.013 |
| Example 120-H | 0.01 |
| Example 120-I | 0.025 |
| Example 120-J | 0.023 |
| Example 120-K | 0.19 |
| Example 120-L | 0.28 |
| Example 120-M | 0.011 |
| Example 120-N | 0.015 |
| Example 120-O | 0.024 |
| Example 120-P | 0.007 |
| Example 120-Q | 0.017 |
| Example 120-R | 0.050 |
| Example 120-S | 0.021 |
| Example 120-T | 0.008 |
| Example 120-U | 0.012 |
| Example 120-V | 0.009 |
| Example 120-W | 0.013 |
| Example 120-X | 0.004 |
| Example 190-L | 3.27 |
| Example 191 | 20.8 |
| Example 209-C | 0.005 |
| Example 209-D | 0.004 |
| Example 209-E | 0.005 |
| Example 210-C | 0.012 |
| Example 211-C | 0.009 |
| Example 212-A | 0.006 |
| Example 212-B | 0.006 |
| Example 213 | 0.005 |
| Example 214-A | 0.010 |
| Example 214-B | 0.005 |
| Example 214-C | 0.008 |
| Example 214-D | 0.018 |
| Example 214-E | 0.008 |
| Example 214-F | 0.003 |
| Example 214-G | 0.022 |
| Example 214-H | 0.7 |
| Example 214-I | 0.007 |
| Example 214-J | 0.009 |
| Example 214-K | 0.015 |
| Example 214-L | 0.012 |
| Example 214-M | 0.02 |
| Example 214-N | 0.005 |
| Example 214-O | 0.071 |
| Example 214-P | 0.014 |
| Example 214-Q | 0.010 |
| Example 214-R | 0.007 |
| Example 215 | 0.008 |
| Example 216-A | 0.007 |
| Example 216-B | 0.056 |
| Example 216-C | 0.006 |
| Example 216-D | 0.002 |
| Example 216-E | 0.006 |
| Example 216-F | 0.031 |
| Example 216-G | 0.007 |
| Example 216-H | 0.005 |
| Example 216-I | 0.013 |
| Example 216-J | 0.003 |
| Example 216-K | 0.002 |
| Example 216-L | 0.024 |
| Example 216-M | 0.013 |
| Example 216-N | 0.024 |
| Example 216-O | 0.078 |
| Example 216-P | 0.013 |
| Example 216-Q | 0.012 |
| Example 216-R | 0.147 |
| Example 216-S | 0.021 |
| Example 216-T | 0.009 |
| Example 216-U | 0.005 |
| Example 216-V | 0.009 |
| Example 216-W | 0.017 |
| Example 216-X | 0.014 |
| Example 288 | 3.43 |
| Example 289 | 0.85 |
| Example 290 | 1.67 |
| Example 291 | 0.19 |
| Example 292-B | 0.07 |
| Example 293 | 0.005 |
| Example 294-A | 0.009 |
| Example 294-B | 0.04 |
| Example 294-C | 0.01 |
| Example 294-D | 0.01 |
| Example 294-E | 0.49 |
| Example 294-F | 0.07 |
| Example 294-G | 4.03 |
| Example 295-C | 0.009 |
| Example 296 | 2.41 |
| Example 297-B | 0.006 |

What is claimed is:

1. A compound according to formula (I) 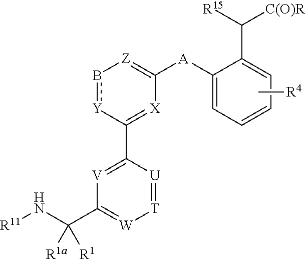
or a salt thereof, wherein
A is —C(O)NH—, —C≡C—, —CH₂CH₂—, S(O)₂N(H)—, or —CHR¹⁰O—, wherein the carbon or sulfur is attached to the ring comprising X, Y and Z; or
A is —N(R¹⁶)CH₂— or —OCH₂—, wherein the nitrogen or oxygen is attached to the ring comprising X, Y and Z; or
R is hydroxy, amino or C₁-C₄alkoxy;
R¹ is hydrogen, phenyl, C₃-C₆cycloalkyl, amido, halo C₁-C₆alkyl or C₁-C₆alkyl optionally substituted by hydroxy, C₃-C₆cycloalkyl, C₁-C₄alkoxy or cyano;
R^{1a} is hydrogen or C₁-C₄alkyl, or CR¹R^{1a} taken in combination form a carbonyl, imine or a 3-6 member cycloalkyl; or
R^{1a} is absent and CR¹ and R¹¹, taken in combination, form a saturated, unsaturated or aromatic 4, 5 or 6 member azacycle;
T is CR² or N;
U is CR¹⁴ or N;
V is CR¹² or N;
W is CR¹³ or N, wherein 0, 1, or 2 of T, U, V and W are N; or
V is N, W is S, T is absent and U is CR¹⁴;
B is CR³ or N;
X is CR⁶ or N;
Y is CR⁵ or N;
Z is CR⁷ or N, wherein 0 or 1 of B, X, Y and Z are nitrogen; or
X is N, B is CR³ and one of Y or Z is S or N(H) and other of Y or Z is absent;
R² is hydrogen, C₁-C₄alkyl or halogen;
R³ is hydrogen, halogen, hydroxy, cyano, amino, NHR⁸, N(R⁸)₂, N(R⁸)C(O)R⁹, —C(O)NHR⁸, —C(O)N(R⁸)₂, OR⁹, S(O)₂R⁹, C₁-C₆alkyl, C₂-C₆alkenyl, C₂-C₆alkynyl, haloC₁-C₆alkyl, C₃-C₆cycloalkyl, phenyl, heterocycloalkyl having 4 to 7 ring atoms and 1, 2, or 3 ring heteroatoms independently selected from N, O and S, and heteroaryl having 5, 6, 9 or 10 ring atoms and 1, 2, or 3 ring heteroatoms independently selected from N, O and S, wherein each heterocycloalkyl, heteroaryl, phenyl is optionally substituted with 0, 1, 2, or 3 substituents independently selected from C₁-C₄alkyl, C₁-C₄alkoxy, C₁-C₄alkoxyC₁-C₄alkyl, halogen, or C₃-C₆cycloalkyl, wherein each heterocycloalkyl or heteroaryl is optionally further substituted by 0 or 1 phenyl groups and wherein each alkyl, alkenyl, alkynyl, haloalkyl and cycloalkyl group is optionally substituted with 0, 1, or 2 substituents independently selected from the group consisting of hydroxy, C₃-C₆cycloalkyl, amino, NHR⁸, N(R⁸)₂, OR⁹, 5 or 6 member heteroaryl having 1 or 2 ring heteroatoms independently selected from N, O and S, and heterocycloalkyl having 4 to 7 ring atoms and 1, 2, or 3 ring heteroatoms independently selected from N, O and S, which heteroaryl or heterocycloalkyl is substituted with 0, 1, or 2 independently selected C₁-C₄alkyl substituents;
R⁴ represents 0, 1, or 2 substituents independently selected from halogen, cyano, C₁-C₄alkyl, C₁-C₄alkoxy, C₃-C₆cycloalkyl, C₃-C₆cycloalkylC₁-C₄-alkyl, C(O)NH₂, NHC(O)C₁-C₄alkyl, CH₂NHC(O)C₁-C₄alkyl, amino, mono- and di-C₁-C₄alkylamino and hydroxyC₁-C₄alkyl;
R⁵ is hydrogen, halogen, cyano, C₁-C₄alkyl or C₁-C₄alkoxy;
R⁶ is hydrogen, halogen, C₁-C₄alkyl or C₁-C₄alkoxy;
R⁷ is hydrogen, halogen, C₁-C₄alkyl, C₁-C₄alkoxy, haloC₁-C₄alkyl or haloC₁-C₄alkoxy, or
R⁷ is phenyl or a 5 or 6 member heteroaryl having 1, 2, or 3 ring heteroatoms selected from N, O or S; each of which is optionally substituted by 0, 1, or 2 substituents selected from C₁-C₄alkyl, aminoC₁-C₄alkyl, hydroxyC₁-C₄alkyl, halogen, C₁-C₄alkoxy, hydroxy, amino or mono- or di-C₁-C₄alkylamino; or
R³ and either R⁵ or R⁷ taken in combination form a —O(CH₂)ₙO— group wherein n is 1 or 2; or
R³ and R⁷ taken in combination with the atoms to which they are attached form a 5 or 6 member aromatic heterocycle having 1 or 2 ring heteroatoms selected from N, O or S and which is optionally substituted with C₁-C₄alkyl, C(O)C₁-C₄alkyl, C(O)NH2, C(O)NHC₁-C₄alkyl, C(O)N(C₁-C₄alkyl)₂, S(O)₂C₁-C₄alkyl, S(O)₂C₃-C₆cycloalkyl, optionally substituted S(O)₂phenyl, where the phenyl is optionally substituted with 0, 1, or 2 C₁-C₄alkyl, or C₁-C₄alkoxy or C₁-C₄alkoxy;
R⁸ is independently selected at each occurrence from the group consisting of hydrogen, C₁-C₆alkyl, haloC₁-C₆alkyl, C₃-C₆cycloalkyl, benzyl, C₁-C₄alkanoyl, benzoyl, phenyl, 4 to 6 member heterocycloalkyl, heteroaryl, wherein C₁-C₆alkyl or haloC₁-C₆alkyl is optionally substituted with C₁-C₄alkoxy, C₃-C₆cycloalkyl, cyano, 4 to 6 member heterocycloalkyl or heteroaryl, wherein phenyl or benzyl are optionally substituted with 0, 1, or 2 substituents selected from C₁-C₄alkyl, haloC₁-C₄alkyl, CH₂CO₂H, C₃-C₆cycloalkyl or C₁-C₄alkoxy, and wherein each heterocycloalkyl or heteroaryl is optionally substituted by 0, 1, or 2 substituents independently selected from C₁-C₄alkyl, CO₂ C₁-C₄alkyl, C(O)NH C₁-C₄alkyl, C₃-C₆cycloalkyl, C₁-C₄alkoxy or a fused benzo ring, and wherein each cycloalkyl is optionally substituted with 0, 1, or 2 independently selected halogen or C₁-C₄alkyl;
R⁹ is independently selected at each occurrence from the group consisting of hydrogen, C₁-C₆alkyl, haloC₁-C₆alkyl, C₃-C₆cycloalkyl, benzyl, benzoyl, phenyl, 4 to 6 member heterocycloalkyl, heteroaryl, wherein C₁-C₆alkyl or haloC₁-C₆alkyl is optionally substituted with C₁-C₄alkoxy, C₃-C₆cycloalkyl, C₃-C₆cycloalkyl substituted with C₁-C₄alkyl, cyano, 4 to 6 member heterocycloalkyl or heteroaryl, wherein phenyl or benzyl are optionally substituted with 0, 1, or 2 substituents selected from C₁-C₄alkyl, CH₂CO₂H, C₃-C₆cycloalkyl or C₁-C₄alkoxy, and wherein each heterocycloalkyl or heteroaryl are optionally substituted by 0, 1, or 2 substituents independently selected from C₁-C₄alkyl, CO₂ C₁-C₄alkyl, C(O)NH C₁-C₄alkyl, C₃-C₆cycloalkyl, C₁-C₄alkoxy or a fused benzo ring, which benzo is optionally substituted with halogen;
R¹⁰ is hydrogen or C₁-C₄alkyl or R⁷ and R¹⁰, taken in combination, form a —(CH₂)ₚ— group or a —O—(CH₂)_{q}— group, wherein p is 2, 3, or 4 and q is 1 or 2;
R¹¹ is hydrogen or C₁-C₄alkyl;
R¹² is hydrogen, halogen, hydroxy, C₁-C₄alkyl or C₁-C₄alkoxy;
R¹³ is hydrogen or halogen;
R¹⁴ is hydrogen or halogen;
R¹⁵ is hydrogen, C₁-C₄alkyl, or NHC(O)R¹⁶; and
R¹⁶ is C₁-C₄alkyl or cyclopropyl each of which is optionally substituted by phenyl; or
R⁷ and R¹⁶ taken in combination for a divalent C₂-C₃ alkylene group.

2. A compound according to formula (Ia) 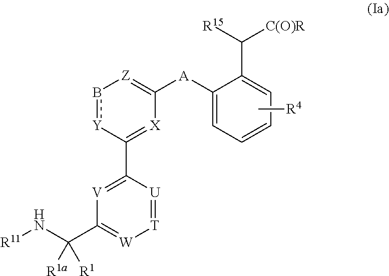
or a salt thereof, wherein
A is —C(O)NH—, —C≡C—, —CH₂CH₂—, S(O)₂N(H)—, or —CHR¹⁰O—, wherein the carbon or sulfur is attached to the ring comprising X, Y and Z; or
A is —NHCH₂— or —OCH₂—, wherein the nitrogen or oxygen is attached to the ring comprising X, Y and Z; or
R is hydroxy, amino or C₁-C₄alkoxy;
R¹ is hydrogen, phenyl, C₃-C₆cycloalkyl, amido, halo C₁-C₄alkyl or C₁-C₄alkyl optionally substituted by hydroxy, C₃-C₆cycloalkyl, C₁-C₄alkoxy or cyano;
R^{1a} is hydrogen or C₁-C₄alkyl, or CR¹R^{1a} taken in combination form a carbonyl (C═O), imine (C═NH) or a 3-6 member cycloalkyl;
R^{1a} is absent and CR¹ and R¹¹, taken in combination, form a saturated, unsaturated or aromatic 4, 5 or 6 member azacycle;
T is CR² or N;
U is CR¹⁴ or N;
V is CR¹² or N;
W is CR¹³ or N, wherein 0, 1, or 2 of T, U, V and W are N; or
V is N, W is S, T is absent and U is CR¹⁴;
B is CR³ or N;
X is CR⁶ or N;
Y is CR⁵ or N;
Z is CR⁷ or N, wherein 0 or 1 of B, X, Y and Z are nitrogen; or
X is N, B is CR³ and one of Y or Z is S or N(H) and other of Y or Z is absent;
R² and R¹⁴ are independently selected from the group consisting of hydrogen and halogen;
R³ is hydrogen, halogen, hydroxy, cyano, amino, NHR⁸, N(R⁸)₂, —C(O)NHR⁸, OR⁹, C₁-C₄alkyl, haloC₁-C₄alkyl, C₃-C₆cycloalkyl, phenyl, heterocycloalkyl having 4 to 7 ring atoms and 1, 2, or 3 ring heteroatoms independently selected from N, O and S, and heteroaryl having 5, 6, 9 or 10 ring atoms and 1, 2, or 3 ring heteroatoms independently selected from N, O and S, wherein each heterocycloalkyl, heteroaryl, phenyl is optionally substituted with 0, 1, 2, or 3 substituents independently selected from C₁-C₄alkyl, C₁-C₄alkoxy, halogen, or C₃-C₆cycloalkyl, wherein each heterocycloalkyl or heteroaryl is optionally further substituted by 0 or 1 phenyl groups and wherein each alkyl, haloalkyl and cycloalkyl group is optionally substituted with 0, 1, or 2 substituents independently selected from the group consisting of hydroxy, C₃-C₆cycloalkyl, amino, NHR⁸, N(R⁸)₂, OR⁹ and heterocycloalkyl having 4 to 7 ring atoms and 1, 2, or 3 ring heteroatoms independently selected from N, O and S;
R⁴ represents 0, 1, or 2 substituents independently selected from halogen, C₁-C₄alkyl, C₁-C₄alkoxy, and hydroxyC₁-C₄alkyl;
R⁵ is hydrogen, halogen, C₁-C₄alkyl or C₁-C₄alkoxy;
R⁶ is hydrogen, halogen, C₁-C₄alkyl or C₁-C₄alkoxy;
R⁷ is hydrogen, halogen, C₁-C₄alkyl, C₁-C₄alkoxy, haloC₁-C₄alkyl or haloC₁-C₄alkoxy, or
R⁷ is phenyl or a 5 or 6 member heteroaryl having 1, 2, or 3 ring heteroatoms selected from N, O or S; each of which is optionally substituted by 0, 1, or 2 substituents selected from C₁-C₄alkyl, aminoC₁-C₄alkyl, hydroxyC₁-C₄alkyl, halogen, C₁-C₄alkoxy, hydroxy, amino or mono- or di-C₁-C₄alkylamino; or
R³ and either R⁵ or R⁷ taken in combination form a —O(CH₂)ₙO— group wherein n is 1 or 2; or
R³ and R⁷ taken in combination with the atoms to which they are attached form a 5 or 6 member aromatic heterocycle having 1 or 2 ring heteroatoms selected from N, O or S and which is optionally substituted with C₁-C₄alkyl, C(O)C₁-C₄alkyl, C(O)NH2, C(O)NHC₁-C₄alkyl, C(O)N(C₁-C₄alkyl)₂, S(O)₂C₁-C₄alkyl, S(O)₂C₃-C₆cycloalkyl, optionally substituted S(O)₂phenyl, where the phenyl is optionally substituted with 0, 1, or 2 C₁-C₄alkyl, or C₁-C₄alkoxy or C₁-C₄alkoxy;
R⁸ is independently selected at each occurrence from the group consisting of hydrogen, C₁-C₄alkyl, haloC₁-C₄alkyl, C₃-C₆cycloalkyl, benzyl, C₁-C₄alkanoyl, benzoyl, phenyl, 4 to 6 member heterocycloalkyl, heteroaryl, wherein C₁-C₄alkyl is optionally substituted with C₁-C₄alkoxy, C₃-C₆cycloalkyl, cyano, 4 to 6 member heterocycloalkyl or heteroaryl, wherein phenyl or benzyl are optionally substituted with 0, 1, or 2 substituents selected from C₁-C₄alkyl, CH₂CO₂H, C₃-C₆cycloalkyl or C₁-C₄alkoxy, and wherein each heterocycloalkyl or heteroaryl are optionally substituted by 0, 1, or 2 substituents independently selected from C₁-C₄alkyl, CO₂ C₁-C₄alkyl, C(O)NH C₁-C₄alkyl, C₃-C₆cycloalkyl, or C₁-C₄alkoxy;
R⁹ is independently selected at each occurrence from the group consisting of hydrogen, C₁-C₄alkyl, haloC₁-C₄alkyl, C₃-C₆cycloalkyl, benzyl, benzoyl, phenyl, 4 to 6 member heterocycloalkyl, heteroaryl, wherein C₁-C₄alkyl is optionally substituted with C₁-C₄alkoxy, C₃-C₆cycloalkyl, cyano, 4 to 6 member heterocycloalkyl or heteroaryl, wherein phenyl or benzyl are optionally substituted with 0, 1, or 2 substituents selected from C₁-C₄alkyl, CH₂CO₂H, C₃-C₆cycloalkyl or C₁-C₄alkoxy, and wherein each heterocycloalkyl or heteroaryl are optionally substituted by 0, 1, or 2 substituents independently selected from C₁-C₄alkyl, CO₂ C₁-C₄alkyl, C(O)NH C₁-C₄alkyl, C₃-C₆cycloalkyl, or C₁-C₄alkoxy;
R¹⁰ is hydrogen or C₁-C₄alkyl or R⁷ and R¹⁰, taken in combination, form a —(CH₂)ₚ— group or a —O—(CH₂)_{q}— group, wherein p is 2, 3, or 4 and q is 1 or 2;
R¹¹ is hydrogen or C₁-C₄alkyl;
R¹² is hydrogen, halogen, hydroxy, C₁-C₄alkyl or C₁-C₄alkoxy;
R¹³ is hydrogen or halogen;
R¹⁴ is hydrogen or halogen; and
R¹⁵ is hydrogen or C₁-C₄alkyl.

3. The compound of claim 2 which compound is represented by formula (II) 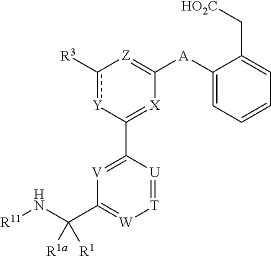
or a salt thereof.

4. The compound of claim 2,
wherein the compound is represented by formula (III): 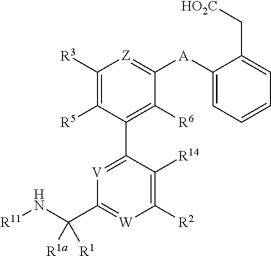
or a salt thereof, wherein
V is CR¹², W is CR¹³ and Z is CR⁷; or
V is N, W is CR¹³ and Z is CR⁷; or
V is CR¹², W is N and Z is CR⁷; or
V is CR¹², W is CR¹³ and Z is N.

5. The compound of claim 2, wherein V is CR¹², W is CR¹³ and Z is CR⁷.

6. The compound of claim 2, wherein V is N, W is CR¹³ and Z is CR⁷; or

7. The compound of claim 2, wherein V is CR¹², W is N and Z is CR⁷.

8. The compound of claim 2, wherein V is CR¹², W is CR¹³ and Z is N.

9. The compound of claim 2, wherein A is CH₂O or —C(O)NH—, wherein the carbon is attached to the ring comprising X, Y and Z.

10. The compound of claim 2, wherein A is CH₂O, wherein the carbon is attached to the ring comprising X, Y and Z.

11. The compound of claim 2, wherein R¹ is hydrogen, C₁-C₄alkyl, hydroxyC₁-C₄alkyl or fluoro C₁-C₄alkyl; and
R^{1a} is hydrogen.

12. The compound of claim 2, wherein R¹ is hydrogen, methyl, hydroxymethyl or fluoromethyl; and
R^{1a} is hydrogen.

13. (canceled)

14. The compound of claim 2, wherein R³ is hydrogen or halogen.

15. The compound of claim 2, wherein R³ is hydrogen or R³ is C₁-C₄alkoxy or C₁-C₄alkylamine, each of which is optionally substituted by C₃-C₆cycloalkyl or a 4 to 6 member saturated heterocycle, which heterocycle has 1 or 2 ring heteroatoms selected from N, O or S.

16. (canceled)

17. The compound of claim 2, wherein R⁵ is hydrogen or halogen.

18. The compound of claim 2, wherein R⁶ is hydrogen or halogen.

19. claim 2, wherein Z is CR⁷ and R⁷ is hydrogen or halogen.

20. The compound of claim 2, wherein R⁵, R⁶ and R⁷ are hydrogen.

21. (canceled)

22. The compound of claim 2, wherein R¹⁴ is hydrogen or halogen.

23. The compound of claim 2, wherein V is CR¹² and R¹² is hydrogen or fluorine.

24. The compound of claim 2, wherein R², R⁵, R⁶, R¹⁰, R¹², R¹³ and R¹⁴ are hydrogen.

25. A pharmaceutical composition comprising one or more pharmaceutically acceptable carriers and a therapeutically effective amount of a compound of claim 2.

26. (canceled)

27. A method of modulating complement alternative pathway activity in a subject, wherein the method comprises administering to the subject a therapeutically effective amount of the compound according to claim 2.

28. A method of treating a disorder or a disease in a subject mediated by complement activation, in particular mediated by activation of the complement alternative pathway, wherein the method comprises administering to the subject a therapeutically effective amount of the compound according to claim 2.

29. The method of claim 28, in which the disease or disorder is selected from the group consisting of age-related macular degeneration, geographic atrophy, diabetic retinopathy, uveitis, retinitis pigmentosa, macular edema, Behcet's uveitis, multifocal choroiditis, Vogt-Koyangi-Harada syndrome, intermediate uveitis, birdshot retino-chorioditis, sympathetic ophthalmia, ocular dicatricial pemphigoid, ocular pemphigus, nonartertic ischemic optic neuropathy, post-operative inflammation, retinal vein occlusion, neurological disorders, multiple sclerosis, stroke, Guillain Barre Syndrome, traumatic brain injury, Parkinson's disease, disorders of inappropriate or undesirable complement activation, hemodialysis complications, hyperacute allograft rejection, xenograft rejection, interleukin-2 induced toxicity during IL-2 therapy, inflammatory disorders, inflammation of autoimmune diseases, Crohn's disease, adult respiratory distress syndrome, myocarditis, post-ischemic reperfusion conditions, myocardial infarction, balloon angioplasty, post-pump syndrome in cardiopulmonary bypass or renal bypass, atherosclerosis, hemodialysis, renal ischemia, mesenteric artery reperfusion after aortic reconstruction, infectious disease or sepsis, immune complex disorders and autoimmune diseases, rheumatoid arthritis, systemic lupus erythematosus (SLE), SLE nephritis, proliferative nephritis, liver fibrosis, hemolytic anemia, myasthenia gravis, tissue regeneration, neural regeneration, dyspnea, hemoptysis, ARDS, asthma, chronic obstructive pulmonary disease (COPD), emphysema, pulmonary embolisms and infarcts, pneumonia, fibrogenic dust diseases, pulmonary fibrosis, asthma, allergy, bronchoconstriction, hypersensitivity pneumonitis, parasitic diseases, Goodpasture's Syndrome, pulmonary vasculitis, Pauci-immune vasculitis, immune complex-associated inflammation, antiphospholipid syndrome, glomerulonephritis and obesity.

30. (canceled)

31. A method of treating glomerulonephritis comprising administering to a subject in need thereof an effective amount of a composition comprising a compound of claim 2.

32-35. (canceled)
